(19) United States
(12) Patent Application Publication
(10) Pub. No.: US 2004/0009907 A1
(43) Pub. Date: Jan. 15, 2004

(54) Proteins and nucleic acids encoding same

(76) Inventors: John P. Alsobrook II, Madison, CT (US); David W. Anderson, Branford, CT (US); Robert A. Ballinger, Newington, CT (US); Ference L. Boldog, North Haven, CT (US); Catherine E. Burgess, Wethersfield, CT (US); Stacie J. Casman, North Haven, CT (US); Karen Ellerman, Branford, CT (US); Esha A. Gangolli, Madison, CT (US); Valerie Gerlach, Branford, CT (US); Jennifer A. Gilbert, Madison, CT (US); Linda Gorman, Branford, CT (US); Xiaojia (Sasha) Guo, Branford, CT (US); Vladimir Y. Gusev, Madison, CT (US); Ramesh Kekuda, Norwalk, CT (US); Li Li, Branford, CT (US); Xiaohong Liu, Branford, CT (US); Uriel M. Malyankar, Branford, CT (US); Charles E. Miller, Guilford, CT (US); Isabelle Millet, Milford, CT (US); Muralidhara Padigaru, Branford, CT (US); Meera Patturajan, Branford, CT (US); Carol E. A. Pena, New Haven, CT (US); John A. Peyman, New Haven, CT (US); Luca Rastelli, Guilford, CT (US); Suresh G. Shenoy, Branford, CT (US); Richard A. Shimkets, Guilford, CT (US); Glennda Smithson, Guilford, CT (US); Kimberly A. Spytek, New Haven, CT (US); David J. Stone, Guilford, CT (US); Raymond J. Taupier JR., East Haven, CT (US); Velizar T. Tchernev, Branford, CT (US); Corine A.M. Vernet, Branford, CT (US); Bryan D. Zerhusen, Branford, CT (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 10/085,198

(22) Filed: Feb. 25, 2002

Related U.S. Application Data
(60) Provisional application No. 60/271,646, filed on Feb. 26, 2001.
(60) Provisional application No. 60/276,401, filed on Mar. 16, 2001.
(60) Provisional application No. 60/311,981, filed on Aug. 13, 2001.
(60) Provisional application No. 60/312,858, filed on Aug. 16, 2001.
(60) Provisional application No. 60/271,840, filed on Feb. 27, 2001.
(60) Provisional application No. 60/277,324, filed on Mar. 20, 2001.
(60) Provisional application No. 60/286,096, filed on Apr. 24, 2001.
(60) Provisional application No. 60/299,695, filed on Jun. 20, 2001.
(60) Provisional application No. 60/315,614, filed on Aug. 29, 2001.
(60) Provisional application No. 60/272,405, filed on Feb. 28, 2001.
(60) Provisional application No. 60/272,410, filed on Feb. 28, 2001.
(60) Provisional application No. 60/272,414, filed on Feb. 28, 2001.
(60) Provisional application No. 60/278,660, filed on Mar. 20, 2001.
(60) Provisional application No. 60/280,234, filed on Mar. 30, 2001.
(60) Provisional application No. 60/272,404, filed on Feb. 28, 2001.
(60) Provisional application No. 60/280,039, filed on Mar. 30, 2001.
(60) Provisional application No. 60/313,280, filed on Aug. 17, 2001.
(60) Provisional application No. 60/322,818, filed on Sep. 17, 2001.
(60) Provisional application No. 60/273,300, filed on Mar. 2, 2001.
(60) Provisional application No. 60/280,818, filed on Apr. 2, 2001.
(60) Provisional application No. 60/288,353, filed on May 3, 2001.
(60) Provisional application No. 60/294,834, filed on May 31, 2001.
(60) Provisional application No. 60/299,845, filed on Jun. 21, 2001.
(60) Provisional application No. 60/272,922, filed on Mar. 2, 2001.
(60) Provisional application No. 60/272,787, filed on Mar. 2, 2001.
(60) Provisional application No. 60/285,754, filed on Apr. 23, 2001.
(60) Provisional application No. 60/303,242, filed on Jul. 5, 2001.
(60) Provisional application No. 60/273,048, filed on Mar. 2, 2001.
(60) Provisional application No. 60/283,443, filed on Apr. 12, 2001.
(60) Provisional application No. 60/291,703, filed on May 17, 2001.

(51) Int. Cl. A61K 38/16; A61K 31/711; C07K 14/435; C07H 21/04

(52) U.S. Cl. 514/12; 530/350; 536/23.1; 514/44

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. S. No. 60/271,646, filed Feb. 26, 2001 (21402-279); U.S. S. No. 60/276,401, filed Mar. 16, 2001 (21402-279 D1); U.S. S. No. 60/311,981, filed Aug. 13, 2001 (21402-279 IFC-01); U.S. S. No. 60/312,858, filed Aug. 16, 2001 (21402-279 IFC-02); U.S. S. No. 60/271,840, filed Feb. 27, 2001 (21402-280); U.S. S. No. 60/277,324, filed Mar. 20, 2001 (21402-280 D1); U.S. S. No. 60/286,096, filed Apr. 24, 2001 (21402-280 A1); U.S. S. No. 60/299,695, filed Jun. 20, 2001 (21402-280 IFC-01); U.S. S. No. 60/315,614, filed Aug. 29, 2001 (21402-280 IFC-02); U.S. S. No. 60/272,405, filed Feb. 28, 2001 (21402-281); U.S. S. No. 60/272,410, filed Feb. 28, 2001 (21402-282); U.S. S. No. 60/272,414, filed Feb. 28, 2001 (21402-283); U.S. S. No. 60/278,660, filed Mar. 20, 2001 (21402-283A); U.S. S. No. 60/280,234, filed Mar. 30, 2001 (21402-283B); U.S. S. No. 60/272,404, filed Feb. 28, 2001 (21402-284); U.S. S. No. 60/280,039, filed Mar. 30, 2001 (21402-284 B1); U.S. S. No. 60/313,280, filed Aug. 17, 2001 (21402-2841FC-01); U.S. S. No. 60/322,818, filed Sep. 17, 2001 (21402-284 C1); U.S. S. No. 60/273,300, filed Mar. 2, 2001 (21402-286); U.S. S. No. 60/280,818, filed Apr. 2, 2001 (21402-286H1); U.S. S. No. 60/288,353, filed May 3, 2001 (21402-286 J1); U.S. S. No. 60/294,834, filed May 31, 2001 (21402-286 F12); U.S. S. No. 60/299,845, filed Jun. 21, 2001 (21402-286 IFC-01); U.S. S. No. 60/272,922, filed Mar. 2, 2001 (21402-287); U.S. S. No. 60/272,787, filed Mar. 2, 2001 (21402-288); U.S. S. No. 60/285,754, filed Apr. 23, 2001 (21402-288 B1); U.S. S. No. 60/303,242, filed Jul. 5, 2001 (21402-288 D1); U.S. S. No. 60/273,048, filed Mar. 2, 2001 (21402-289); U.S. S. No. 60/283,443, filed Apr. 12, 2001 (21402-289 A); and U.S. S. No. 60/291,703, filed May 17, 2001 (21402-289 B); each of which is incorporated by reference in their entirety.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1-NOV91 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 2n-1, wherein n is any integer between 1 and 107. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2n, where n is any integer between 1 and 107. The nucleic acid can be, for example, a genomic DNA fragment or a cDNA molecule that includes the nucleic acid sequence of any of SEQ ID NOS:2n-1.

Also included hi the invention is an oligonucleotide, e.g., an oligonucleotide which includes at least 6 contiguous nucleotides of a NOVX nucleic acid (e.g., SEQ ID NOS:2n-1) or a complement of said oligonucleotide.

Also included in the invention are substantially purified NOVX polypeptides (SEQ ID NOS:2n). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, hypercoagulation, hemophilia, idiopathic thrombocytopenic purpura, heart failure, secondary pathologies caused by heart failure and hypertension, hypotension, angina pectoris, myocardial infarction, tuberous sclerosis, scleroderma, transplantation, autoimmune disease, lupus erythematosus, viral/bacterial/parasitic infections, multiple sclerosis, autoimmume disease, allergies, immunodeficiencies, graft versus host disease, asthma, emphysema, ARDS, inflammation and modulation of the immune response, viral pathogenesis, aging-related disorders, Th1 inflammatory diseases such as rheumatoid arthritis, multiple sclerosis, inflammatory bowel diseases, AIDS, wound repair, obesity, diabetes, endocrine disorders, anorexia, bulimia, renal artery stenosis, interstitial nephritis, glomerulonephritis, polycystic kidney disease, systemic, renal tubular acidosis, IgA nephropathy, nephrological disesases, hypercalceimia, Lesch-Nyhan syndrome, Von Hippel-Lindau (VHL) syndrome, trauma, regeneration (in vitro and in vivo), Hirschsprung's disease, Crohn's Disease, appendicitis, endometriosis, laryngitis, psoriasis, actinic keratosis, acne, hair growth/loss, allopecia, pigmentation disorders, myasthenia gravis, alpha-mannosidosis, beta-mannosidosis, other storage disorders, peroxisomal disorders such as zellweger syndrome, infantile refsum disease, rhizomelic chondrodysplasia (chondrodysplasia punctata, rhizomelic), and hyperpipecolic acidemia, osteoporosis, muscle disorders, urinary retention, Albright Hereditary Ostoeodystrophy, ulcers, Alzheimer's disease, stroke, Parkinson's disease, Huntington's disease, cerebral palsy, epilepsy, Lesch-Nyhan syndrome, multiple sclerosis, ataxia-telangiectasia, behavioral disorders, addiction, anxiety, pain, neuroprotection, Stroke, Aphakia, neurodegenerative disorders, neurologic disorders, developmental defects, conditions associated with the role of GRK2 in brain and in the regulation of chemokine receptors, encephalomyelitis, anxiety, schizophrenia, manic depression, delirium, dementia, severe mental retardation and dyskinesias, Gilles de la Tourette syndrome, leukodystrophies, cancers, breast cancer, CNS cancer, colon cancer, gastric cancer, lung cancer, melanoma, ovarian cancer, pancreatic cancer, kidney cancer, colon cancer, prostate cancer, neuroblastoma, and cervical cancer, Neoplasm; adenocarcinoma, lymphoma; uterus cancer, benign prostatic hypertrophy, fertility, control of growth and development/differentiation related functions such as but not limited maturation, lactation and puberty, reproductive malfunction, and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1 | CG57602-01 | 1 | 2 | DJ0751H13.1 Protein |
| 2 | CG57558-01 | 3 | 4 | Mac25/IGFBP7 |
| 3 | CG57560-01 | 5 | 6 | Calmodulin Binding Protein Kinase |
| 4A | CG57547-01 | 7 | 8 | TRANSIENT RECEPTOR |
| | | | | POTENTIAL-RELATED |
| | | | | PROTEIN |
| 4B | CG57547-02 | 9 | 10 | TRANSIENT RECEPTOR |
| | | | | POTENTIAL-RELATED |
| | | | | PROTEIN |
| 5 | CG57609-01 | 11 | 12 | Epsin-3 |
| 6 | CG57611-01 | 13 | 14 | CD22, a B lymphocyte-restricted |
| | | | | adhesion molecule |
| 7 | CG57595-01 | 15 | 16 | MEGF8 |
| 8A | CG57452-01 | 17 | 18 | protocadherin |
| 8B | CG57452-02 | 19 | 20 | protocadherin |
| 9 | CG57625-01 | 21 | 22 | protocadhcrin |
| 10 | CG57553-01 | 23 | 24 | T01CI.3 |
| 11A | CG57488_01 | 25 | 26 | alpha-macroglobulin |
| 11B | CG57488_02 | 27 | 28 | alpha-macroglobulin |
| 11C | CG57488_03 | 29 | 30 | alpha-macroglobulin |
| 12A | CG57526_01 | 31 | 32 | orphan transporter |
| 12B | CG57526-02 | 33 | 34 | orphan transporter |
| 13 | CG57570-01 | 35 | 36 | cation transporter |
| 14 | CG57593-01 | 37 | 38 | ABC transporter |
| 15 | CG57652-01 | 39 | 40 | Diacylglycerol Kinase Alpha |
| 16 | CG57562-01 | 41 | 42 | Cation-transporting ATPasc |
| 17 | CG55914-01 | 43 | 44 | ACYL-COA DESATURASE |
| 18 | CG57328-01 | 45 | 46 | MYO-INOSITOL-1(OR 4)- |
| | | | | MONOPHOSHATASE |
| 19 | CG57358-01 | 47 | 48 | spinster |
| 20 | CG57695-01 | 49 | 50 | casein |
| 21 | CG57654-01 | 51 | 52 | Gamma-Aminobutyric-Acid |
| | | | | Receptor Gamma-2 Subunit |
| | | | | Precursor (GABA-A Receptor) |
| 22 | CG57724-01 | 53 | 54 | Carboxylesterase |
| 23 | CG57730-01 | 55 | 56 | MAT-1 oncogene |
| 24 | CG5755-01 | 57 | 58 | VACUOLAR PROTON-ATPASE |
| | | | | SUBUNIT H |
| 25 | CG57503-01 | 59 | 60 | MEGF7 |
| 26 | CG57456-01 | 61 | 62 | COP-COATED VESICLE |
| | | | | MEMBRANE PROTEIN P24 |
| | | | | PRECURSOR |
| 27 | CG57658-01 | 63 | 64 | Connexin |
| 28 | CG57662-01 | 65 | 66 | Connexin |
| 29 | CG57664-01 | 67 | 68 | the MHC CLASS I ANTIGEN |
| 30 | CG57666-01 | 69 | 70 | the MHC CLASS I ANTIGEN |
| 31 | CG57668-01 | 71 | 72 | the MHC CLASS I ANTIGEN |
| 32 | CG57660-01 | 73 | 74 | RETINOIC ACID RECEPTOR |
| | | | | RESPONDER |
| 33 | CG57672-01 | 75 | 76 | PHOSPHATIDYLINOSITOL 4- |
| | | | | PHOSPHATE 5-KINASE |
| 34 | CG57680-01 | 77 | 78 | Cyclophilin-type peptidyl-prolyl |
| | | | | cis-trans isomerase |
| 35 | CG57670-01 | 79 | 80 | pyruvate kinase |
| 36A | CG57149_01 | 81 | 82 | Cis/Trans Peptidyl Prolyl Isomerase |
| 36B | CG57149_02 | 83 | 84 | Cis/Trans PeptidyI Prolyl Isomerase |
| 37 | CG57151_01 | 85 | 86 | Cis/Trans Peptidyl Prolyl Isomerase |
| 38 | CG57153_01 | 87 | 88 | Cis/Trans Peptidyl Prolyl Isomerase |
| 39 | CG57155_01 | 89 | 90 | Cis/Trans Peptidyl Prolyl Isomerase |
| 40 | CG57157_01 | 91 | 92 | Cis/Trans Peptidyl Prolyl Isomerase |
| 41 | CG57159_01 | 93 | 94 | Cis/Trans Peptidyl Prolyl Isomerase |
| 42A | CG57226_01 | 95 | 96 | Cis/Trans Peptidyl Prolyl Isomerase |
| 42B | CG57226-02 | 97 | 98 | Cis/Trans Peptidyl Prolyl Isomerase |
| 43 | CG57538-01 | 99 | 100 | Ceruloplasmin |
| 44A | CG57623-01 | 101 | 102 | Leucine Rich Repeat |
| 44B | CG57623-02 | 103 | 104 | Leucine Rich Repeat |
| 45A | CG57656-01 | 105 | 106 | Ig/Fibronectin domain |
| 45B | CG57656-02 | 107 | 108 | Ig/Fibronectin domain |
| 46 | CG57682-01 | 109 | 110 | G2/MITOTIC-SPECIFIC CYCLIN |
| | | | | B2 |
| 47 | CG57764-01 | 111 | 112 | ALR |
| 48 | CG57713-01 | 113 | 114 | SODIUM/BILE ACID |
| | | | | COTRANSPORTER |
| 49 | CG57721-01 | 115 | 116 | Prestin |
| 50 | CG57787_01 | 117 | 118 | Sulfate Transporter |
| 51 | CG57785_01 | 119 | 120 | Sulfate Transporter |
| 52 | CG57748-01 | 121 | 122 | N-acetylgalactosaminyltransferase |
| 53 | CG57693-01 | 123 | 124 | Protein Kinase |
| 54 | CG57707-01 | 125 | 126 | Leucinc-rich glioma-inactivated |
| | | | | protein precursor |
| 55 | CG57306-01 | 127 | 128 | Anion exchanger |
| 56 | CG57348_01 | 129 | 130 | PR_SET_domain protein |
| 57 | CG57650-01 | 131 | 132 | NONMUSCLE MYOSIN HEAVY |
| | | | | CHAIN B |
| 58 | CG57766-01 | 133 | 134 | Plasma retinol binding protein |
| 59 | CG57566-01 | 135 | 136 | HIV-I inducer of short transcripts |
| | | | | binding protein |
| 60A | CG57574-01 | 137 | 138 | Beta tectorin |
| 60B | CG57574-02 | 139 | 140 | Beta tectorin |
| 60C | CG57574-03 | 142 | 142 | Beta tectorin |
| 61 | CGS7505-01 | 143 | 144 | KIAA1125 |
| 62A | CG57473-01 | 145 | 146 | Zinc-finger protein BOP |
| 62B | CG57473-02 | 147 | 148 | Zinc-finger protein BOP |
| 63 | CG57777_01 | 149 | 150 | Hypothetical secreted protein |
| 64 | CG57779_01 | 151 | 152 | Hypothetical secreted protein |
| 65 | CG57781_01 | 153 | 154 | Hypothetical secreted protein |
| 66 | G57783_01 | 155 | 156 | Hypothetical secreted protein |
| 67A | CG57823-01 | 157 | 158 | ACYLTRANSFERASE |
| 67B | CG57823-02 | 159 | 160 | ACYLTRANSFERASE |
| 68 | CG57801-01 | 161 | 162 | guanine nucleotide exchange factor |
| 69A | CG57719-01 | 163 | 164 | Aspartate Aminotransferase |
| 69B | CG57719-02 | 165 | 166 | Aspartate Aminotransferase |
| 70 | CG57462-01 | 167 | 168 | KIAA1337 |
| 71 | CG57584-01 | 169 | 170 | ZONA PELLUCIDA |
| | | | | GLYCOPROTEIN I PRECURSOR |
| 72 | CG56761-01 | 171 | 172 | Ankyrin repeat containing protein |
| 73 | CG57313-01 | 173 | 174 | GPCR |
| 74 | CG57315_01 | 175 | 176 | GPCR |
| 75 | CG57317_01 | 177 | 178 | GPCR |
| 76 | CG57321_01 | 179 | 180 | GPCR |
| 77 | CG57419_01 | 181 | 182 | GPCR |
| 78 | CG57425-01 | 183 | 184 | GPCR |
| 79 | CG57753-01 | 185 | 186 | GPCR |
| 80 | CG56766-01 | 187 | 188 | GPCR |
| 81A | CG57847-01 | 189 | 190 | GPCR |
| 81B | CG57847-02 | 191 | 192 | GPCR |
| 82 | CG57845-01 | 193 | 194 | GPCR |
| 83 | CG57843-01 | 195 | 196 | GPCR |
| 84A | CG57841-01 | 197 | 198 | GPCR |
| 84B | CG57841-02 | 199 | 200 | GPCR |
| 85 | CG57839-01 | 201 | 202 | GPCR |
| 86 | CG57837-01 | 203 | 204 | GPCR |
| 87 | CG56763-01 | 205 | 206 | GPCR |
| 88 | CG56753-01 | 207 | 208 | GPCR |
| 89 | CG57670-01 | 209 | 210 | GPCR |
| 90 | CG57676-01 | 211 | 212 | GPCR |
| 91 | CG57678-01 | 213 | 214 | GPCR |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Table A indicates homology of NOVX nucleic acids to known protein families. Thus, the nucleic acids and polypeptides, antibodies and related compounds according to the invention corresponding to a NOVX as identified in column 1 of Table A, will be useful in therapeutic and diagnostic applications implicated in, for example, pathologies and disorders associated the the known protein families identified in column 5 of Table A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogeniesis, cell differentiation, cell proliferation, hematopoiesis, wound healing and angiogenesis.

Additional utilities for the NOVX nucleic acids and polypeptides according to the invention are disclosed herein.

NOV1

A disclosed NOV1 nucleic acid of 12660 nucleotides (also referred to as CG57602-01) encoding a DJ0751H13.1 PROTEIN-like protein is shown in Table 1A. An open reading frame was identified beginning with an ATG initiation codon at nucleotides 1-3 and ending with a TAA codon at nucleotides 12658-12660. The start and stop codons are in bold letters.

TABLE 1A
NOV1 nucleotide sequence.

| | |
|---|---|
| ATGCTACTCCCTGCCCTCCTCTTTGGGATGGCGTGGGCCCTGGCTGACGCGCGGTGGTGT | (SEQ ID NO:1) |
| GAGTGGACAGAGACCATCCGTGTGGAGGAGGAAGTGGCACCCCGTCAGGAGGACCTGGTA | |
| CCCTGTGCCAGCCTCGACCATTACAGCCGCCTGGGCTGGCGGCTGGACCTGCCCTGGAGT | |
| GGCCGCTCGGGGCTTACCCGGTCCCCAGCGCCTGGGCTCTGTCCTATCTACAAACCTCCA | |
| GAAACCCGGCCTGCCAAGTGGAACCGGACAGTGAGGACTTGTTGCCCAGGCTGGGGGGGC | |
| GCCCACTGCACTGAGGCCCTTGCCAAAGCCAGTCCTGAAGGCCACTGCTTTGCCATGTGG | |
| CAGTGCCAGCTACAGGCAGGCTCAGCTAATGCCTCAGCAGGAAGCCTGGAGGAGTGCTGC | |
| GCCCGGCCCTGGGGACGAAGCTGGTGGGATGGCAGCTCCCAGGCCTGCCGCAGCTGCTCC | |
| AGCCGACACCTGCCAGGCAGTGCCTCTTCTCCAGCCCTCCTGCAGCCCCTGGCAGGGGCT | |
| GTGGGCCAGCTCTGGAGCCACCACCAGCGTCCCTCGGCCACCTGTGCCTCCTGGTCGGGC | |
| TTCCACTACCGCACCTTTGATGGCCGCCACTATCACTTCCTGGGCCGCTGCACCTACCTG | |
| CTGGCGGGTGCTGCGGACTCCACCTGGGCTGTCCACCTAACACCCGGGGACCGCTGCCCC | |
| CAGCCTGGACACTGTCAGCGGGTCCAGGTGACTATGGGACCCGAGGAGGTGCTGATCCAG | |
| GCTGGAAATGTGTCTGTGAAGGGGCAGCTGGTACCTGAAGGGCAGTCTTGGCTGCTCCAC | |
| GGGCTGAGCCTGCAATGGCTGGGGGACTGGCTGGTGCTGTCAGGAGGCCTGGGGGTCGTG | |
| GTGCGGCTGGACAGGACTGGCTCCATCTCCATCTCTGTGGACCACGAGCTCTGGGGACAG | |
| ACACAAGGCCTCTGTGGGCTCTACAATGGCTGGCCAGAGGATGACTTCATGGAGCCAGGC | |
| GGAGGGCTGGCCATGTTAGCAGCCACCTTTGGAAATTCCTGGAGGCTCCCTGGCTCGGAG | |
| GTTTCCCCCGCTGAGTACCACGAGGCCTGTCTCTTTGCCTACTGCGCAGGGGCCATGGCA | |
| GGCAGTGGGCAAGAGGGGCGGCAGCAGGCTGTTTGTGCCACCTTTGCCAGCTATGTCCAG | |
| GCCTGTGCCAGGCGGCACATCCACATTCGCTGGAGGAAGCCTGGCTTCTGCGAGCGCCTG | |
| TGCCCCGGGGGCCAGCTCTACTCCGACTGCGTCTCCCTCTGCCCACCCAGCTGCGAGGCG | |
| GTGGGTCAGGGAGAGGAGGAGTCCTGCAGGGAAGAGTGTGTGAGTGGCTGTGAGTGCCCG | |
| CGAGGCCTCTTCTGGAATGGCACCCTCTGTGTGCCTGCTGCCCACTGCCCCTGCTACTAC | |
| TGCCGCCAGCGCTATGTACCCGGTGACACCGTGCGCCAGCTGTGTAACCCCTGCGTGTGC | |
| AGGGATGGCCGCTGGCACTGTGCCCAGGCACTGTGCCCCGCCGAGTGTGCAGTGGGTGGG | |
| GACGGGCACTACCTCACCTTCGATGGGCGGAGCTACTCCTTCTGGGGTGGTCAAGGTTGC | |
| CGCTACAGCCTGGTGCAGCCCTATCCTTCCACGTGCCCCACCCCCACCATCAGACCCCCT | |
| GTTCCAGGAGCTGTGCTGGTCAATGGGCAGGATGTGGGCTTGCCCTGGATTGGCGCTGAG | |
| GGCCTCAGTGTGCGCCGAGCTTCCTCTGCCTTTCTGCTGCTGCGCTGGCCTGGGGCCCAG | |
| GTGCTCTGGGGACTGTCTGACCCTGTAGCCTACATCACCCTGGACCCCCGCCATGCCCAC | |
| CAGGTGCAGGGTCTGTGTGGCACCTTCACCCAGAACCAGCAGGACGACTTCCTGACACCA | |
| GCCGGAGATGTGGAAACTAGCATTGCTGCCTTTGCTAGCAAGTTCCAGGTGGCCGGCAAG | |
| GGAAGATGCCCCTCTGAGGACAGTGCCCTGCTGTCTCCCTGCACCACCCACTCCCAGCGC | |
| CACGCCTTCGCAGAGGCGGCCTGTGCCATCCTGCACAGCTCTGTCTTCCAGGAATGCCAC | |
| AGGCTGGTGGACAAAGAGCCATTCTATCTGCGCTGCCTGGCAGCCGTGTGTGGCTGTGAT | |
| CCCGGCAGTGACTGCCTGTGCCCGGTGCTGTCTGCCTATGCGCGTCGCTGTGCCCAGGAA | |
| GGTGCCTCACCTCCCTGGAGGAACCAGACCCTCTGCCCTGTTATGTGTCCTGGTGGCCAG | |
| GAGTACCGAGAGTGTGCCCCAGCATGCGGTCAACACTGCGGGAAACCAGAGGACTGTGGA | |
| GAGCTGGGCAGCTGTGTGGCTGGTTGTAACTGTCCTCTGGGGCTGCTGTGGGACCCTGAG | |
| GGCCAGTGTGTGCCCCCCAGCTTGTGCCCCTGCCAGCTCGGAGCCCGTCGCTATGCCCCT | |
| GGCAGTGCCACCATGAAGGAGTGCAACCGCTGGGAGCTTGTCTATGCCCCTGGTGCCTGT | |
| CTCCTCACCTGTGACAGCCCCAGCGCCAATCACTCCTGCCCTGCAGGCAGTACTGATGGC | |
| TGTGTCTGTCCACCAGGCACGGTGCTGCTGGACGAGCGCTGTGTGCCTCCTGACCTCTGT | |
| CCCTGCCGTCACAGTGGGCAGTGGTACCTGCCCAACGCCACCATCCAGGAAGACTGCAAC | |
| GTTTGCGTGTGCCGGGGCCGGCAGTGGCACTGCACAGGCCAGCGGCGCAGTGGGCGGTGC | |
| CAGGCATCAGGCGCCCCCCACTATGTGACATTTGACGGACTGGCCTTCACCTATCCTGGG | |
| GCCTGCGAGTATCTGCTGGTGCGAGAGGCCAGTGGCCTATTCACAGTCTCTGCCCAGAAC | |
| CTGCCCTGTGGGGCCAGCGGTCTCACCTGCACCAAAGCGCTGGCCGTGCGTCTGGAGGCC | |
| ACTGTTGTGCACATGCTCAGAGGGACTCGGGTCCTGGTGCAACTGTCCCCTCAGTTCCGT | |
| GGTCGCGTGGCTGGGCTGTGTGGTGACTTTGATGGAGATGCCAGTAATGATCTGCGGAGC | |
| CGCCAGGGCGTCCTGGAGCCCACAGCTGAACTGGCTGCCCACTCCTGGCGCCTCAGCCCC | |
| CTCTGCCCTGAGCCAGGAGACCTGCCACACCCCTGCACGATGAACACACACCGGGCTGGT | |
| TGGGCTCGGGCCCGCTGTGGGGCGCTGCTGCAGCCGCTCTTCACATTATGCCACGCGGAG | |
| GTCCCCCCGCAGCAGCACTATGAGTGGTGCCTGTATGACGCCTGCGGCTGCGACTCGGGG | |
| GGTGACTGTGAGTGCCTCTGCTCGGCCATTGCCACCTATGCAGATGAGTGTGCCCGGCAT | |
| GGGCACCACGTGCGCTGGCGTAGCCAGGAGCTCTGCTGTCTACACCAGACACCCTGTGCC | |
| CTCCATGGTGGTCACCTTGGCCAGCCAGCCTGGTGTGGTTGCATCCTTTTGCCCTTGTGC | |
| CTCAGTGATCCAAGGCTCTCTCCTCTCCACCCAGCCCTGCAGTGTGAAGGGGGACAGGTA | |
| TATGAGGCCTGTGGCCCCACGTGTCCCCCCACCTCCCATGAGCAGCATCCTGAGCCCGGG | |
| TGGCACTGCCAGGTGGTGGCCTGTGTGGAGGGCTGCTTCTGCCCCGAGGGGACTCTGCTG | |
| CACGGACGCTGTGTGCTCAGCACGTGCCAGGAAGGTCAATGGCATTGTGGGGGTGACGGT | |
| GGCCACTGTGAGGAGCTTGTGCCTGCCTGTGCACAGGGAGAGGCCCTGTGCCAAGAGAAT | |
| GGGCACTGTGTGCCCCATGGGTGGCTTTGTGACAACCAGGACGACTGTGGCGATGGCTCT | |
| GATGAGGAGGGTGAGTGTCTTTGCCCATGCGTGGAAGCGACAGGGTTGGTCAGTCCTTGC | |
| ACATGTTGTGCCGCCCCAGGCTGTGGGGAGGGGCAGATGACTTGCAGCTCCGGCCACTGC | |
| CTGCCCCTGGCCCTGCTCTGTGACCGCCAGGATGACTGTGGAGATGGCACGGATGAGCCG | |
| AGCTATCCGTGCCCCCAGGGCTTGCTGGCCTGTGCCGATGGACGCTGCCTGCCGCCGGCC | |
| CTGCTCTGCGATGGGCATCCTGACTGTCTGGATGCCGCCGACGAGGAGTCCTGTCTGGGG | |
| CAGGTGACCTGCGTCCCCGGGGAGGTGTCCTGTGTTGATGGCACCTGCCTGGGGGCCATC | |
| CAGCTGTGTGACGGAGTCTGGGACTGCCCAGATGGAGCCGATGAGGGGCCGGGACACTGC | |
| CCCCTACCTTCTCTGCCCACACCTCCTGCCAGCACCTTGCCTGGCCCCTCCCCAGGCTCC | |
| CTGGACACTGCGTCAAGTCCCCTGGCCAGCGCCAGCCCTGCGCCACCCTGCGGCCCCTTC | |
| GAGTTTCGGTGCGGCAGCGGCGAGTGCACCCCGCGGGGCTGGCGCTGCGACCAGGAGGAA | |
| GACTGCGCCGACGGCAGCGACGAGCGCGGCTGCGGAGGGCCCTGCGCGCCGCACCACGCG | |
| CCCTGCGCCCGCGGCCCTCACTGCGTGTCCCCCGAGCAGCTGTGCGACGGCGTGCGGCAG | |
| TGTCCCGACGGCTCGGACGAGGGCCCCGACGCCTGCGTTGAGGCTCCCGCGCCCCCGGCC | |
| ATGCGCGGCCCCCCTGGCCAAGCCGGCGGGCCCACCTCTTCCCGAGCGCCATCCCCACCT | |
| TCGCCTCCTGAGGCACAGGGAGAGGGCAGGAAGGGACAGGAGCGGAGCAGGACACATCTC | |
| ACAGTGCCCGCAGGCTCCACCCAGCTGCCTCTGTGCCCTGGCCTCTTTCCCTGTGGTGTG | |
| GCTCCGGGGCTGTGCCTGACCCCTGAGCAGCTCTGTGATGGGATCCCAGACTGTCCCCAG | |
| GGCGAGGACGAGCTGGACTGCGGGGGGCTGCCAGCCCTGGGAGGCCCCAACAGGACAGGG | |
| CTTCCCTGCCCAGAATACACCTGCCCCAATGGCACCTGCATAGGCTTCCAGCTGGTGTGT | |
| GATGGGCAGCCTGACTGTGGAAGGCCAGGGCAGGTGGGCCCCTCCCCAGAAGAGCAGGGT | |
| TGTGGGGCCTGGGGCCCCTGGAGCCCATGGGGGCCCTGCAGCCGGACGTGTGGGCCCTGG | |
| GGCCAGGGCCGGAGCCGCCGCTGCTCCCCACTCGGCCTCCTGGTGCTACAGAACTGCCCA | |
| GGGCCTGAGCACCAGTCTCAGGCCTGCTTCACGGCAGCCTGCCCAGTGGACGGTGAATGG | |
| AGCACCTGGTCCCCCTGGTCTGTGTGCTCTGAGCCGTGCAGGGGCACCATGACGCGGCAA | |
| CGGCAGTGCCACTCACCCCAGAATGGGGGCCGCACCTGTGCTGCACTGCCCGGAGGCCTG | |
| CACAGCACCCGCCAGACCAAGCCTTGCCCTCAGGACGGCTGCCCCAATGCCACTTGCTCT | |
| GGGGAGCTGATGTTCCAGCCCTGTGCCCCCTGCCCACTGACCTGTGATGACATCTCTGGC | |
| CAGGTCACGTGCCCACCTGATTGGCCCTGCGGCAGCCCGGGCTGCTGGTGCCCAGAAGGG | |
| CAGGTGCTGGGCAGCGAGGGGTGGTGTGTGTGGCCCCGGCAGTGCCCCTGCCTGGTGGAC | |
| GGTGCCCGCTACTGGCCTGGGCAACGCATCAAGGCCGACTGCCAGCTCTGCATCTGCCAA | |
| GACGGACGGCCCCGACGCTGCCGACTCAACCCGGACTGCGCTGGTGAGGCCCTTCCCTCG | |
| GGGTCCCTAGTCCTCTCCCTGGACCGCCCAGCTGCACATCCACCACCTCCTTCAGGCTCT | |
| GACTGTTGGCCCTCCCTCAGTGGACTGTGGCTGGTCCTCCTGGTCACCCTGGGCCAAGTG | |
| CCTGGGCCCCTGTGGAAGCCAGAGCATCCAGTGGTCCTTCCGGAGCTCCAACAACCCCCG | |
| CCCCTCCGGCCGAGGTCGCCAGTGCCGTGGCATCCACCGCAAGGCACGCAGACGGAGCCC | |
| TGTGAGGGGTGTGAGCATCAGGGCCAGGTCCACCGTGTCGGGGAACGCTGGCATGGGGGC | |
| CCCTGCAGGGTGTGCCAGTGTCTGCACAACCTCACCGCACACTGCTCACCCTACTGCCCG | |
| CTCGGCAGCTGCCCCCAGGGCTGGGTCTTGGTGGAGGGGACGGGAGAATCATGCTGCCAC | |
| TGTGCCCTACCTGGAGAGAACCAGACGGTCCAGCCCATGGCCACTCCTGCCGCAGCTCCG | |
| GCTCCCAGTCCCCAGATCAGATTCCCTTTGGCCACTTACATTCTGCCTCCGTCAGGAGGC | |
| TCCTGCCGCCCTCTGTCCTCCCCTACTCCAGCCTGTCTCTCTCTTCTGCACCCAGACCCC | |
| TGCTATTCTCCCCTGGGGCTGGCCGGACTGGCTGAGGGGAGTCTGCATGCATCGTCCCAG | |
| CAGCTGGAACACCCCACCCAGGCTGCCCTCCTGGGGGCTCCCACCCAGGGGCCCAGCCCT | |
| CAGGGATGGCACGCTGGAGGGGATGCTTATGCCAAGTGGCACACTCGGCCCCATTACCTG | |
| CAGCTGGACCTGCTTCAGCCTCGGAACCTCACTGGCATCCTAGTGCCGGAGACTGGCTCC | |
| TCCAACGCATATGCCAGCAGCTTCTCACTCCAGTTCAGCAGCAATGGTCTACACTGGCAT | |
| GACTATCGTGACCTCCTGCCTGGCATCTTGCCCCTGCCCAAGGTATCACCCGCCCAAGGC | |
| CGATGGGGCCAGCAGCCCACCATGCCCTTTTGTGGGTTCCATAGTCTTTGTCCCCAAGGG | |
| CCTTCCAGTGTCCCCGAGGGGCATGGCCTGCATTCGATGCTTGTTGAATACCTGCTTTTC | |
| CCCAGAAACTGGGATGACCTGGACCCTGCCGTATGGACTTTCGGCCGCATGGTGCAGGCG | |
| AGGTTTGTCAGGGTGTGGCCCCACGATGTCCACCACAGCGATGTCCCCCTGCAGGTGGAG | |
| CTGCTGGGCTGCGAGCCAGGGGTTGGACTCCGCTGTGCCAGTGGTGAGTGTGTCCTGAGA | |
| GGGGGCCCTTGTGACGGTGTTCTGGACTGCGAGGATGGCTCGGATGAGGAGGGCTGTGTG | |
| TTGCTGCCTGAGGGCACTGGCAGGTATACTGTGGCCGGCCGTGCAGCTCACGCCCTTGGC | |
| CTGGCCTTTGAGGGGACAGCCATGTGGGAGGGGCCCGGCACTGCCTTCACCCCCAAGGTG | |
| CCCAGACCCTGCATGCTGAGGAGCTGCAGCCGGGGCCTGGCAGAGACTGAGCACTGGCCC | |
| CCTGGGCAGGAATCCCCCACGTCCCCGACAGAAGCCTGGGATACCCTCTCGAGGGCCCCC | |
| ACCTTCCTTTCCTGGGAAGGGGAGCTGGGCAAGCCTCACCTCCCTCTACCTACACCCACA | |
| GAGACAAGGCCCGTGAGTCCTGGCCCAGCCTCCGGGGTGCCTCACCATGGGGAATCTGTG | |
| CAGATGGTGACCACCACCCCCATACCCCAGATGGAGGCCAGGACCCTGCCACCAGGTATG | |
| GCAGCTGTGACGGTGGTGCCCCCACACCCTGTGACTCCAGCGACCCCTGCTGGTAAGATG | |
| CCAAGCCCTTCTACCTGTGTGCAAGGCCTCTGCCAGAGCGTCGCCCCAGGACCCTTCCCA | |
| CCTGTGCAGTGTGGCCCCGGCCAGACGCCCTGTGAGGTGCTGGGCTGCGTGGAACAGGCG | |
| CAGGTGTGTGATGGCAGGGAGGATTGCCTCGACGGCTCCGACGAGAGGCACTGCGGTGAG | |
| CTGCTGGAGGGCCTGCTGTCCTGTGGGGCCCTCTGTTCCCCGAGCCAGCTGAGCTGTGGC | |
| AGCGGGGAGTGTCTGTCTGCTGAGCGGCGCTGTGACCTGCGGCCTGACTGCCAGGATGGC | |
| TCGGACGAGGATGGCTGTGTGGACTGCGTGCTGGCCCCCTGGTCTGTCTGGAGCAGCTGC | |
| AGCCGCAGCTGTGGCCTGGGCCTCACCTTCCAGCGCCAGGAGCTGCTGCGGCCTCCTCTG | |
| CCAGGGGGCAGCTGCCCGCGTGACCGGTTCCGAAGCCAGTCCTGCTTTGTGCAGGCCTGC | |
| CCAGTGGCTGGGGCATGGGCCATGTGGGAGGCCTGGGGACCCTGCAGCGTCTCCTGCGGG | |
| GGTGGCCATCAGAGTCGCCAGAGAAGCTGTGTGGACCCCCCACCCAAGAATGGCGGTGCC | |
| CCCTGCCCCGGGGCCTCCCAAGAGAGGGCACCCTGCGGCTTGCAGCCCTGCTCAGGTGGC | |
| ACAGGTAAGGGGGTGCTGGGCTGGGGACACGGAGGGAGCACTGTGGGCACGGGGCGATTG | |
| GGCCTCCCAGCACCTAGGCTCACCTGGTGCCCATCCCCCACCAGACTGCGAGCTGGGCCG | |
| TGTGTATGTGAGTGCCGATCTGTGCCAGAAGGGGCTGGTGCCCCCATGCCCACCCTCCTG | |
| CCTGGATCCCAAGGCCAACAGAAGCTGCAGTGGGCATTGTGTGGAAGCCTCCCCTCCCTG | |
| CTGTGCCCCTTGGGGCTGAGTGCCCTCTTCCACATACTCCCAGGATGCCGCTGTCCCCCG | |
| GGGCTCCTTCTGCATGACACTCGCTGCCTGCCCCTCTCTGAGTGCCCCTGCCTGGTGGGC | |
| GAAGAGCTGAAGTGGCCAGGGGTGTCCTTCCTCCTGGGCAACTGCAGCCAATGCGTGTGT | |
| GAGAAGGGGGAGTTGCTGTGCCAACCAGGGGGCTGCCCCCTGCCCTGCGGCTGGTCAGCC | |
| TGGTCCTCCTGGGCTCCCTGCGACCGCTCCTGTGGCTCTGGAGTGAGGGCCAGGTTCAGG | |
| TCTCCCTCCAACCCTCCGGCAGCCTGGGGGGGTGCCCCGTGTGAAGGTGACCGGCAGGAA | |
| CTGCAGGGCTGCCACACAGTGTGTGGGACAGGTATAGCCGGGAGCCTGGGTGCAGGAGTC | |
| CCCCCTTCCTCCTCCCAATTCTGTACCCTGAGAACACATGGGATGGGACCCACTGACCAC | |
| TCTACGTGGGGGATTGAGGTGTTCGGCTGGACGCCCTGGACTTCCTGGTCCTCCTGCTCC | |
| CAAAGCTGCCTTGCCCCGGGAGGGGGCCCTGGCTGGCGCAGTCGTTCCCGACTCTGCCCC | |
| AGCCCTGGGGATTCATCCTGCCCAGGAGATGCCACCCAGGAGGAGCCCTGCAGCCCCCCT | |
| ATAGAGTGTACGGGCTTCTGCGCCCCCGGCTGCACCTGCCCCCCTGGTCTTTTCCTGCAC | |
| AATGCTAGCTGCCTGCCCCGCAGCCAGTGCCCCTGCCAGCTGCACGGGCAGCTCTATGCA | |
| TCAGGAGCAATGGCTCGCCTGGACTCCTGCAACAACTGCACCTGTGTCTCTGGTAAGATG | |
| GCATGCACCTCGGAGCGCTGCCCAGTGGCCTGTGGCTGGAGTCCCTGGACCCTGTGGAGT | |
| CTCTGTAGCTGCAGCTGCAACGTGGGCATTCGGCGCCGCTTCCGGGCAGGCACTGCACCC | |
| CCAGCTGCCTTTGGGGGTGCTCAGTGCCAAGGCCCCACCATGGAGGCTGAATTCTGCAGC | |
| CTGCGGCCATGTCCAGGGCCAGTGCCTGGCATGTGTCCCAGGGACAAGCAGTGGCTGGAC | |
| TGTGCCCAGGGCCCTGCCTCTTGTGCAGAGCTCAGCGCCCCAAGAGGGACTAACCAGACC | |
| TGCCACCCTGGCTGCCACTGCCCCTCTGGGATGCTTCTGCTGGTGAGCCCACGTGGTCAC | |
| CCTGGACCCCTTGGAGCCAGTGTTCAGCCTCCTGTGGCCCTGCCCGGTGCCATCGGCACC | |
| GGTTCTGTGCCAGGAGCTGGGGGATGGGGTCCATGGGGGCCCTGGTCCCACTGTAGCCGG | |
| AGCTGTGGGGGAGGCCTGCGGAGCCGGACCCGGGCCTGTGACCAGCCCCCACCCCAGGGC | |
| CTGGGGGATTACTGCGAGGGGCCACGGGCACAGGGGGAGGTCTGCCAGGCTCTGCCCTGC | |
| CCAGTGACCAACTGCACTGCCATTGAAGGGGCCGAGTATAGCCCCTGTGGCCCTCCGTGC | |
| CCTCGCTCCTGTGATGACCTAGTGCACTGCGTGTGGCGCTGCCAGCCTGGCTGCTACTGC | |
| CCACCAGGCCAGGTACTGAGTTCCAACGGGGCCATCTGCGTGCAGCCGGGTCACTGCAGC | |
| TGCCTGGACCTGCTGACCGGGCAGCGGCACCATCCGGGTGCTCGGCTGGCAAGGCCTGAC | |
| GGCTGCAACCACTGCACCTGCCTGGAGGGGAGGCTGAACTGCACAGACCTGCCCTGCCCA | |
| GACTGCGGGGGTGGCCAGAGTCTGCATCCCTGTGGGCAGCCCTGCCCCCGCTCCTGCCAG | |
| GACCTGTCCCCTGGGAGTGTGTGCCAGCCAGGCTCTGTGGGCTGCCAGCCCACTTGTGGG | |
| TGCCCCCTGGGCCAGCTCTCCCAGGACGGGCTGTGCGTGCCCCCAGCCCACTGCCGCTGC | |
| CAGTACCAGCCTGGAGCCATGGCCCCCTCCTTTGTCCCCAGCACCTGTGTGGCAGGCATT | |
| CTGCAATGCCAGGAGGTGCCTGACTGCCCGGACCCTGGGGTGTGGAGCTCTTGGGGCCCT | |
| TGGGAAGACTGCAGTGTTTCGTGTGGGGGCGGGGAGCAGCTGCGCTCCCGGCGCTGTGCT | |
| CGTCCTCCCTGCCCAGGGCCTGCCCGCCAGAGCCGCACATGCAGCACACAGGTCTGCAGA | |
| GAGGCAGGCTGCCCGGCTGGCCGCCTGTACCGTGAATGCCAGCCCGGCGAGGGATGCCCC | |
| TTCTCCTGCGCCCACGTCACGCAGCAGGTGGGCTGCTTCTCTGAGGGCTGCGAGGAGGGC | |
| TGCCACTGCCCCGAGGGCACCTTCCAGCACCGCCTGGCCTGTGTGCAGGAGTGCCCTTGT | |
| GTGCTGACAGCCTGGCTGCTGCAGGAGCTGGGAGCCACCATAGGTGACCCTGGTCAGCCC | |
| CTCGGGCCTGGAGATGAGCTGGACTCAGGCCAGACACTTCGTACAAGCTGTGGCAACTGC | |
| TCGTGTGCACACGGGAAGCTGTCTTGCTCCCTGGACGACTGCTTCGAGGCCGATGGTGGT | |
| TTCGGTCCCTGGAGCCCGTGGGGCCCGTGCTCCCGCTCCTGTGGAGCGCTGGGCACCCGT | |
| ACCCGCAGCCGCCAGTGTGTGCTCACCATGCCCACCCTCAGTGAGCTGCCCGTGTGCCCT | |
| GGCCCTGGCTGTGGGGCTGGGAACTGTTCCTGGACCTCCTGGGCCCCGTGGGAACCTTGC | |
| TCCCGCAGCTGCGGAGTGGGCCAGCAGCGCCGCCTGCGGGCATACCGTCCCCCTGGGCCC | |
| GGCGGGCACTGGTGCCCCAACATCCTTACTGCCTACCAAGAGCGCCGCTTCTGCAACCTG | |
| CGACCCTGCCCAGAGGCCGGCTGCCCAGCAGGCATGGAGGTGGTCACCTGTGCCAACCGC | |
| TGCCCCCGCCGCTGCTCAGACCTCCAGGAGGGAATTGTGTGTCAGGACGACCAGGTCTGC | |
| CAGAAGGGCTGCCGCTGCCCAAAGGGGTCCCTGGAGCAGGATGGTGGCTGCGTGCCAATT | |
| GGGCACTGTGACTGCACCGATGCCCAGGGCCACAGCTGGGCCCCGGGGAGCCAGCACCAG | |
| GATGCCTGCAACAACTGCTCATGCCAAGCTGGGCAGCTCTCCTGCACGGCTCAGCCCTGC | |
| CCGCCTCCCACCCACTGTGCCTGGAGCCACTGGTCGGCCTGGAGTCCCTGCAGCCACTCA | |
| TGCGGGCCCAGAGGGCAGCAGAGCCGCTTCCGCTGCGGCCCGGGCCTGGCCTCTCGCTCT | |
| GGGTCCTGCCCCTGCCTGATGGCCAAGGCCGACCCCACCTGCAACAGCACCTTCCTCCAC | |
| CTGGACACCCAGGGCTGCTACTCAGGGCCCTGCCCAGACTCATGCCAGTGGAGTCTGTGG | |
| GGGCCATGGAGCCCCTGCCAGGTGCCCTGCAGTGGGGGGTTCAGGCTACGCTGGAGAGAG | |
| GCAGAGGCCCTCTGTGGAGGAGGCTGCCGGGAGCCATGGGCTCAAGACAGAAAGCTGCAA | |
| CGGAGGGCCCTGCCCAGCACCTGTGTCAACGAGTCCCTGGTGTGCCCACACCAGGAGTGT | |
| CCAGTCCTTGGGCCTTGGTCAGCCTGGAGCAGTTGCTCGGCCCCCTGTGGTGGGGGCACT | |
| ATGGAGCGACATCGGACTTGTGAGGGGGGTCCTGGGGTGGCACCATGCCAGGCCCAGGAC | |
| ACAGAGCAACGGCAGGAGTGTAACCTGCAGCCCTGCCCTGAGTGCCCCCCTGGCCAGGTG | |
| CTTAGTGCCTGTGCCACCTCATGCCCGTGCCTCTGCTGGCATCTGCAGCCTGGTGCCATC | |
| TGTGTGCAGGAGCCCTGCCAGCCTGGCTGTGGCTGCCCTGGAGGGCAGCATTCTCTGCCC | |
| TGGGGCCTCACCCTGACCCTGGAAGAGCAGGCCCAGGAGCTGCCCCCAGGGACTGTGCTC | |
| ACCCGGAACTGCACCCGCTGTGTCTGCCACGGTGGAGCCTTCAGCTGCTCCCTCGTTGAC | |
| TGTCAGGGTGAGATAGTGCCCCCTGGGGAAACGTGGCAGCAGGTGGCCCCGGGGGAGCTG | |
| GGGCTCTGCGAGCAGACGTGCCTGGAGATGAACGCCACAAAGACCCAGAGTAACTGCAGT | |
| TCAGCTCGAGCCTCGGGCTGCGTGTGCCAGCCCGGGCACTTCCGCAGCCAGGCAGGCCCC | |
| TGCGTCCCCGAAGACCACTGCGAGTGCTGGCACCTTGGGCGTCCCCACCTGGTGAGACAC | |
| CGAACCCCCTCTGCTACCACTCACCCATTCCTGACCCCAAGCCTCCCCATCTGTCTGTAA | |

In a search of public sequence databases, the NOV1 nucleic acid sequence, located on chromsome 8 has 606 of 779 bases (77%) identical to a gb:GENBANK-ID:BTSCOSPON|acc:X93922.1 mRNA from Bos taurus (B.taurus mRNA for SCO-spondin protein). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., B.taurus mRNA for SCO-spondin protein, matched the Query NOV1 sequence purely by chance is 2.4e-152. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.nicbi.nltii.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNN”) or the letter “X” in protein sequences (e.g., “XXXXXXXXX”). Low-complexity regions can result in high scores that reflect compositional bias rather than significant position-by-position alignment. (Wootton and Federhen, Methods Enzymol 266:554-571, 1996).

The disclosed NOV1 polypeptide (SEQ ID NO:2) encoded by SEQ ID NO:1 has 4219 amino acid residues and is presented in Table 113 using the one-letter amino acid code. Signal P, Psort and/or Hydropathy results predict that NOV1 has a signal peptide and is likely to be localized extracellularly with a certainty of 0.5087. The most likely cleavage site for NOV1 is between positions 17 and 18.

TABLE 1B
Encoded NOV1 protein sequence.

| | |
|---|---|
| MLLPALLFGMAWALADGRWCEWTETIRVEEEVAPRQEDLVPCASLDHYSRLGWRLDLPWS | (SEQ ID NO:2) |
| GRSGLTRSPAPGLCPIYKPPETRPAKWNRTVRTCCPGWGGAHCTEALAKASPEGHCFAMW | |
| QCQLQAGSANASAGSLEECCARPWGRSWWDGSSQACRSCSSRHLPGSASSPALLQPLAGA | |
| VGQLWSQHQRPSATCASWSGFHYRTFDGRHYHFLGRCTYLLAGAADSTWAVHLTPGDRCP | |
| QPGHCQRVQVTMGPEEVLIQAGNVSVKGQLVPEGQSWLLHGLSLQWLGDWLVLSGGLGVV | |
| VRLDRTGSISISVDHELWGQTQGLCGLYNGWPEDDFMEPGGGLAMLAATFGNSWRLPGSE | |
| VSPAEYHEACLFAYCAGAMAGSGQEGRQQAVCATFASYVQACARRHIHIRWRKPGFCERL | |
| CPGGQLYSDCVSLCPPSCEAVGQGEEESCREECVSGCECPRGLFWNGTLCVPAAHCPCYY | |
| CRQRYVPGDTVRQLCNPCVCRDGRWHCAQALCPAECAVGGDGHYLTFDGRSYSFWGGQGC | |
| RYSLVQPYPSTCPTPTIRPPVPGAVLVNGQDVGLPWIGAEGLSVRRASSAFLLLRWPGAQ | |
| VLWGLSDPVAYITLDPRHAHQVQGLCGTFTQNQQDDFLTPAGDVETSIAAFASKFQVAGK | |
| GRCPSEDSALLSPCTTHSQRHAFAEAACAILHSSVFQECHRLVDKEPFYLRCLAAVCGCD | |
| PGSDCLCPVLSAYARRCAQEGASPPWRNQTLCPVMCPGGQEYRECAPACGQHCGKPEDCG | |
| ELGSCVAGCNCPLGLLWDPEGQCVPPSLCPCQLGARRYAPGSATMKECNRWELVYAPGAC | |
| LLTCDSPSANHSCPAGSTDGCVCPPGTVLLDERCVPPDLCPCRHSGQWYLPNATIQEDCN | |
| VCVCRGRQWHCTGQRRSGRCQASGAPHYVTFDGLAFTYPGACEYLLVREASGLFTVSAQN | |
| LPCGASGLTCTKALAVRLEGTVVHMLRGTRVLVQLSPQFRGRVAGLCGDFDGDASNDLRS | |
| RQGVLEPTAELAAHSWRLSPLCPEPGDLPGPCTMNTHRAGWARARCGALLQPLFTLCHAE | |
| VPPQQHYEWCLYDACGCDSGGDCECLCSAIATYADECARHGHHVRWRSQELCCLHQTPCA | |
| LHGGHLGQPAWCGCILLPLCLSDPRLSPLHPALQCEGGQVYEACGPTCPPTCHEQHPEPG | |
| WHCQVVACVEGCFCPEGTLLHGRCVLSTCQEGQWHCGGDGGHCEELVPACAEGEALCQEN | |
| GHCVPHGWLCDNQDDCGDGSDEEGECLCPCVEATGLVSPCTCCAAPGCGEGQMTCSSGHC | |
| LPLALLCDRQDDCGDGTDEPSYPCPQGLLACADGRCLPPALLCDGHPDCLDAADEESCLG | |
| QVTCVPGEVSCVDGTCLGAIQLCDGVWDCPDGADEGPGHCPLPSLPTPPASTLPGPSPGS | |
| LDTASSPLASASPAPPCGPFEFRCGSGECTPRGWRCDQEEDCADGSDERGCGGPCAPHHA | |
| PCARGPHCVSPEQLCDGVRQCPDGSDEGPDACVEAPAPPAMRGPPGQAGGPTSSRAPSPP | |
| SPPEAQGEGRKGQERSRTHLTVPAGSTQLPLCPGLFPCGVAPGLCLTPEQLCDGIPDCPQ | |
| GEDELDCGGLPALGGPNRTGLPCPEYTCPNGTCIGFQLVCDGQPDCGRPGQVGPSPEEQG | |
| CGAWGPWSPWGPCSRTCGPWGQGRSRRCSPLGLLVLQNCPGPEHQSQACFTAACPVDGEW | |
| STWSPWSVCSEPCRGTMTRQRQCHSPQNGGRTCAALPGGLHSTRQTKPCPQDGCPNATCS | |
| GELMFQPCAPCPLTCDDISGQVTCPPDWPCGSPGCWCPEGQVLGSEGWCVWPRQCPCLVD | |
| GARYWPGQRIKADCQLCICQDGRPRRCRLNPDCAGEALPSGSLVLSLDRPAAHPPPPSGS | |
| DCWPSLSGLWLVLLVTLGQVPGPLWKPEHPVVLPELQQPPPLRPRSPVPWHPPQGTQTEP | |
| CEGCEHQGQVHRVGERWHGGPCRVCQCLHNLTAHCSPYCPLGSCPQGWVLVEGTGESCCH | |
| CALPGENQTVQPMATPAAAPAPSPQIRFPLATYILPPSGGSCRPLSSPTPACLSLLHPDP | |
| CYSPLGLAGLAEGSLHASSQQLEHPTQAALLGAPTQGPSPQGWHAGGDAYAKWHTRPHYL | |
| QLDLLQPRNLTGILVPETGSSNAYASSFSLQFSSNGLHWHDYRDLLPGILPLPKVSPAQG | |
| RWGQQPTMPFCGFHSLCPQGPSSVPEGHGLHSMLVEYLLFPRNWDDLDPAVWTFGRMVQA | |
| RFVRVWPHDVHHSDVPLQVELLGCEPGVGLRCASGECVLRGGPCDGVLDCEDGSDEEGCV | |
| LLPEGTGRYTVAGRAAHALGLAFEGTANWEGPGTAFTPKVPRPCMLRSCSRGLAETEHWP | |
| PGQESPTSPTEAWDTLSRAPTFLSWEGELGKPHLPLPTPTETRPVSPGPASGVPHHGESV | |
| QMVTTTPIPQMEARTLPPGMAAVTVVPPHPVTPATPAGKMPSPSTCVQGLCQSVAPGPFP | |
| PVQCGPGQTPCEVLGCVEQAQVCDGREDCLDGSDERHCGELLEGLLSCGALCSPSQLSCG | |
| SGECLSAERRCDLRPDCQDGSDEDGCVDCVLAPWSVWSSCSRSCGLGLTFQRQELLRPPL | |
| PGGSCPRDRFRSQSCFVQACPVAGAWANWEAWGPCSVSCGGGHQSRQRSCVDPPPKNGGA | |
| PCPGASQERAPCGLQPCSGGTGKGVLGWGHGGSTVGTGRLGLPAPRLTWCPSPTRLRAGP | |
| CVCECRSVPEGAGAPMPTLLPGSQGQQKLQWALCGSLPSLLCPLGLSALFHILPGCRCPP | |
| GLLLHDTRCLPLSECPCLVGEELKWPGVSFLLGNCSQCVCEKGELLCQPGGCPLPCGWSA | |
| WSSWAPCDRSCGSGVRARFRSPSNPPAAWGGAPCEGDRQELQGCHTVCGTGIAGSLGAGV | |
| PPSSSQFCTLRTHGMGPTDHSTWGIEVFGWTPWTSWSSCSQSCLAPGGGPGWRSRSRLCP | |
| SPGDSSCPGDATQEEPCSPPIECTGFCAPGCTCPPGLFLHNASCLPRSQCPCQLHGQLYA | |
| SGAMARLDSCNNCTCVSGKMACTSERCPVACGWSPWTLWSLCSCSCNVGIRRRFRAGTAP | |
| PAAFGGAECQGPTMEAEFCSLRPCPGPVPGMCPRDKQWLDCAQGPASCAELSAPRGTNQT | |
| CHPGCHCPSGMLLLVSPRGHPGPLGASVQPPVALPGATGTGSVPGAGGWGPWGPWSHCSR | |
| SCGGGLRSRTRACDQPPPQGLGDYCEGPRAQGEVCQALPCPVTNCTAIEGAEYSPCGPPC | |
| PRSCDDLVHCVWRCQPGCYCPPGQVLSSNGAICVQPGHCSCLDLLTGQRHHPGARLARPD | |
| GCNHCTCLEGRLNCTDLPCPDCGGGQSLHPCGQPCPRSCQDLSPGSVCQPGSVGCQPTCG | |
| CPLCQLSQDGLCVPPAHCRCQYQPGAMAPSFVPSTCVAGILQCQEVPDCPDPGVWSSWGP | |
| WEDCSVSCGGGEQLRSRRCARPPCPGPARQSRTCSTQVCREAGCPAGRLYRECQPGEGCP | |
| FSCAHVTQQVGCFSEGCEEGCHCPEGTFQHRLACVQECPCVLTAWLLQELGATIGDPGQP | |
| LGPGDELDSGQTLRTSCGNCSCAHGKLSCSLDDCFEADGGFGPWSPWGPCSRSCGGLGTR | |
| TRSRQCVLTMPTLSELPVCPGPGCGAGNCSWTSWAPWEPCSRSCGVGQQRRLRAYRPPGP | |
| GGHWCPNILTAYQERRFCNLRACPEAGCPAGMEVVTCANRCPRRCSDLQEGIVCQDDQVC | |
| QKGCRCPKGSLEQDGGCVPIGHCDCTDAQGHSWAPGSQHQDACNNCSCQAGQLSCTAQPC | |
| PPPTHCAWSHWSAWSPCSHSCGPRCQQSRFRCGPGLASRSGSCPCLMAKADPTCNSTFLH | |
| LDTQGCYSGPCPDSCQWSLWGPWSPCQVPCSGGFRLRWREAEALCGGGCREPWAQDRKLQ | |
| RRALPSTCVNESLVCPHQECPVLGPWSAWSSCSAPCGGGTMERHRTCEGGPGVAPCQAQD | |
| TEQRQECNLQPCPECPPGQVLSACATSCPCLCWHLQPGAICVQEPCQPGCGCPGGQHSLP | |
| WGLTLTLEEQAQELPPGTVLTRNCTRCVCHGGAFSCSLVDCQGEIVPPGETWQQVAPGEL | |
| GLCEQTCLEMNATKTQSNCSSARASGCVCQPGHFRSQAGPCVPEDHCECWHLGRPHLVRH | |
| RTPSATTHPFLTPSLPICL | |

A search of sequence databases reveals that the NOV1 amino acid sequence has 4056 of 4056 amino acid residues (100%) identical to, and 4056 of 4056 amino acid residues (100%) similar to, the 4123 amino acid residue ptnr:SPTREMBL-ACC:O75851 protein from Homo sapiens (Human) (WUGSC:H_DJ0751H13.1 PROTEIN) (E=0.0). Public amino acid databases include the GenBank databases, SwissProt, PDB and PIR.

The disclosed NOV1 polypeptide has homology to the amino acid sequences shown in the BLASTP data listed in Table 1C.

TABLE 1C
BLAST results for NOV1

| Gene Index/ | | Length | | Positives | |
|---|---|---|---|---|---|
| Identifier | Protein/Organism | (aa) | Identity (%) | (%) | Expect |
| gi\|3638957\| | sco-spondin- | 4123 | 4056/4056 | 4056/4056 | 0.0 |
| gb\|AAC36301.1\| | mucin-like; | | (100%) | (100%) | |
| (AC004877) | similar. to P98167 | | | | |
| | (PID: g1711548); | | | | |
| | [Homo sapiens] | | | | |
| gi\|17465206\| | similar to sco- | 1098 | 879/1060 | 890/1060 | 0.0 |
| ref\|XP_069674.1\| | spondin-mucin- | | (82%) | (83%) | |
| (XM_069674) | like; similar to | | | | |
| | P98167 | | | | |
| | (PID: g1711548); | | | | |
| | (H. sapiens) | | | | |
| gi\|3808095\| | SCO-spondin [Bos | 870 | 600/990 | 633/990 | 0.0 |
| emb\|CAA69867.1\| | taurus] | | (60%) | (63%) | |
| (Y08560) | | | | | |
| gi\|1711548\| | SCO-spondin, | 867 | 582/1035 | 635/1035 | 0.0 |
| sp\|P98167\| | bovine | | (56%) | (61%) | |
| SSPO BOVIN | | | | | |
| gi\|5823595\| | SCO-spondin, | 564 | 360/494 | 395/494 | e−173 |
| emb\|CAB53760.1\| | bovine | | (72%) | (79%) | |
| (AJ132107) | | | | | |

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV1 as disclosed in Tables 1D-H, were collected from the Conserved Domain Database (CDD) with Reverse Position Specific BLAST analyses. This BLAST analysis software samples domains found in the Smart and Pfam collections. For Tables 1G-10 and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Tables 1D-H list the domain descriptions from DOMAIN analysis results for NOV1. This indicates that the NOV1 sequence has properties similar to those of other proteins known to contain this domain.

TABLE 1D
Domain Analysis of NOV1

gnl|Pfam|pfam00754, F5_F8_type_C, F5/8 type C domain.
This domain is also known as the discoidin (DS) domain family.
CD-Length = 145 residues, 100.0% aligned
Score = 115 bits (288), Expect = 5e−26

TABLE 1E
Domain Analysis of NOV1

gnl|Pfam|pfam00094, vwd, von Willebrand factor type D domain.
CD-Length = 158 residues, 99.4% aligned
Score = 97.1 bits (240), Expect = 2e−20

TABLE 1F
Domain Analysis of NOV1

gnl|Smart|smart00231, FA58C, Coagulation factor ⅝ C-terminal
domain, discoidin domain; Cell surface-attached carbohydrate-binding
domain, present in eukaryotes and assumed to have horizontally
transferred to eubacterial genomes.
CD-Length = 135 residues, 99.3% aligned
Score = 63.2 bits (152), Expect = 3e−10

TABLE 1G
Domain Analysis of NOV1

gnl|Smart|smart00209, TSP1, Thrombospondin type 1 repeats; Type 1
repeats in thrombospondin-1 bind and activate TGF-beta.
CD-Length = 51 residues, 100.0% aligned
Score = 53.1 bits (126), Expect = 3e−07

TABLE 1H
Domain Analysis of NOV1

gnl|Pfam|pfam00057, ldl_recept_a, Low-density lipoprotein receptor
domain class A
CD-Length = 39 residues, 94.9% aligned
Score = 50.4 bits (119), Expect = 2e−06

The disclosed NOV1 protein contains a thrombospondin type I repeat domain which are found in the thrombospondin protein and is repeated 3 times. A number of proteins involved in the complement pathway (properdin, C6, C7, C8A, C8B, C9) as well as extracellular matrix protein like mindin, F-spondin, SCO-spondin and even the circumsporozoite surface protein 2 and TRAP proteins of Plasmodium contain one or more instance of this repeat. It has been involved in cell-cell interraction, inhibition of angiogenesis, apoptosis. The intron-exon organisation of the properdin gene confirms the hypothesis that the repeat might have evolved by a process involving exon shuffling. A study of properdin structure provides some information about the structure of the thrombospondin type I repeat.

The disclosed NOV1 nucleic acid of the invention encoding a DJ0751H13.1 PROTEIN-like protein includes the nucleic acid whose sequence is provided in Table 1A or a fragment thereof. The invention also includes a mutant or variant nucleic acid any of whose bases may be changed from the corresponding base shown in Table 1A while still encoding a protein that maintains its DJ0751H13.1 PROTEIN like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 27 percent of the bases may be so changed.

The disclosed NOV1 protein of the invention includes the DJ0751H13.1 PROTEIN-like protein whose sequence is provided in Table 1B. The invention also includes a mutant or variant protein any of whose residues may be changed from the corresponding residue shown in Table 1B while still encoding a protein that maintains its DJ0751H13.1 PROTEIN-like activities and physiological functions, or a functional fragment thereof. In the mutant or variant protein, up to about 0 percent of the residues may be so changed.

The invention further encompasses antibodies and antibody fragments, such as F_{ab} or (F_{ab})₂, that bind immunospecifically to any of the proteins of the invention.

The above defined information for this invention suggests that this DJ0751H13.1 PROTEIN-like protein (NOV1) may function as a member of a “DJ0751H113.1 PROTEIN family”. Therefore, the NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV1 nucleic acids and proteins of the invention are useful in potential therapeutic applications implicated in cancer including but not limited to various pathologies and disorders as indicated below. For example, a cDNA encoding the DJ0751H13.1 PROTEIN-like protein (NOV1) may be useful in gene therapy, and the DJ0751H13.1 PROTEIN-like protein (NOV1) may be useful when administered to a subject in need thereof. By way of nonlimiting example, the compositions of the present invention will have efficacy for treatment of patients suffering from cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, Alzheimer's disease, stroke, tuberous sclerosis, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, cancers, and/or other pathologies and disorders. For example, a cDNA encoding the transmembrane receptor DJ0751H13.1 PROTEIN-like protein may be useful in transmembrane receptor DJ0751H13.1 PROTEIN therapy, and the transmembrane receptor DJ0751H13.1 PROTEIN-like protein may be useful when administered to a subject in need thereof. By way of nonlimiting example, the compositions of the present invention will have efficacy for treatment of patients suffering from cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, Alzheimer's disease, stroke, tuberous sclerosis, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, cancers, and other diseases, disorders and conditions of the like. Also since this gene is expressed at a measurably higher level in several cancer cell lines (including breast cancer, CNS cancer, colon cancer, gastric cancer, lung cancer, melanoma, ovarian cancer and pancreatic cancer), it may be useful in diagnosis and treatment of these cancers. The NOV1 nucleic acid encoding the DJ0751H13.1 PROTEIN-like protein of the invention, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV2

A disclosed NOV2 nucleic acid of 893 nucleotides (also referred to as CG57558-01) encoding a Mac25/IGFBP7-like protein is shown in Table 2A. Putative untranslated regions upstream and/or downstream from the coding region, if any, are underlined, and the start and stop codons are in bold letters.

TABLE 2A
NOV2 nucleotide sequence.

(SEQ ID NO:3)

GTACCTTAAAGACAACAAACAAGCAAACACAACTTATAATTAAAAAACAT
GCAAAGGGCTCACCTTCCACTTCCTTCTGGTCCTGCTCCTCTTCTTCCTC
CTCTCCTGCCTCCTCTTCTCCCTCTTCCATCAGACCTTCTGGGGCCCCT
TTCAATAAGCAGCTGCTGGCCGGCCAGCCCTTGGGGGCAGGGCTGGAAC
CGGGGCAGGGGAGGCTGCGGGGCCACTCGCTGGAGAGGCAAACAGGAAG
GACTGCCCCCTGAGCGCCAGGCTTCGGGCCCGGGAATCGCCGCCGCCGC
CGCCGCAGAGCTGCAGCTCGGGGCCGAGGGTAAGGAGGCGAGCCGGGAG
CGGGAGGCCCGGGAGAGCTCCGCGGGTCCCCGCGCCCAGTCCCCAGCCG
CGCCCCGACCCCGCCGCCCCGGGCCTAACGCGGCCGGCGAGGCCTACGC
GGCGGCCGCCGTCACCGTGCTGGAGCCGCCGGCCTCCGACCCCGAGCTG
CAGCCCGCCGAGCGCCCGCTGCCATCGCCGGGGTCCGGGGAGGGCGCCC
CGGTCTTCCTCACGGGGCCTCGATCCCAGTGGGTGCTGCGGGGGGCGGA
GGTGGTGCTGACGTGCCGGGCGGGGGGCCTCCCCGAGCCCACACTGTAC
TGGGAGAAGGACGGGATGGCCCTGGACGAAGTGTGGGACAGCAGCCACT
TCGCGCTCCAGCCGGGCCGCGCCGAGGACGGCCCCGGCGCGAGCCTGGC
ACTGCGCATCCTGGCGGCTCGGCTGCCGGATTCCGGCGTCTACGTGTGC
CACGCCCGCAACGCGCACGGCCACGCGCAGGCGGGGGCGCTGCTCCAGG
TGCTGACCCCCACCTTCCTGCCGCCAAGACAGCCCTAACCAAGGCCCAG
AAAGGGTAG

In a search of public sequence databases, the NOV2 nucleic acid sequence, located on chromsome 2 has has 564 of 779 bases (72%) identical to a gb:GENBANK-ID:S56581|acc:S56581.1 mRNA from Rattus sp. (alpha inhibin gene {5′ region} [rats, Genomic, 2141 nt]). Public nucleotide databases include all GenBank databases and the GeneSeq patent database.

The disclosed NOV2 polypeptide (SEQ ID NO:4) encoded by SEQ ID NO:3 has 274 amino acid residues and is presented in Table B using the one-letter amino acid code. Signal P, Psort and/or Hydropathy results predict that NOV2 has a signal peptide and is likely to be localized extracellularly with a certainty of 0.3700. The most likely cleavage site for a NOV2 peptide is between amino acids 32 and 33.

TABLE 2B
Encoded NOV protein sequence.

(SEQ ID NO:4)

MQRAHLPLPSGPAPLLPPLLPPLLPVPSDLLGPLSISSCWPASPWGQGWN
RGRGGCGATRWRGKQEGLPPERQASGPGIAAAAAAELQLGAEGKEASRER
EARESSAGPRAQSPAAPRPRRPGPNAAGEAYAAAAVTVLEPPASDPELQP
AERPLPSPGSGEGAPVFLTGPRSQWVLRGAEVVLTCRAGGLPEPTLYWEK
DGMALDEVWDSSHFALQPGRAEDGPGASLALRILAARLPDSGVYVCHARN
AHGHAQAGALLQVLTPTFLPPRQP

A search of sequence databases reveals that the NOV2 amino acid sequence has 80 of 266 amino acid residues (30%) identical to, and 112 of 266 amino acid residues (42%) similar to, the 277 amino acid residue ptnr:SPTREMBL-ACC:Q07822 protein from Homo sapiens (Human) (MAC25 PROTEIN). Public amino acid databases include the GenBank databases, SwissProt, PDB and PIR.

NOV2 is expressed in at least brain, ovary, breast, testis. This information was derived by determining the tissue sources of the sequences that were included in the invention including but not limited to SeqCalling sources, Public EST sources, Literature sources, and/or RACE sources.

The disclosed NOV2 polypeptide has homology to the amino acid sequences shown in the BLASTP data listed in Table 2C.

TABLE 2C
BLAST results for NOV2

| Gene Index/ | | Length | Identity | Positives | |
|---|---|---|---|---|---|
| Identifier | Protein/Organism | (aa) | (%) | (%) | Expect |
| gi\|14734071\| | KIAA0657 protein [Homo | 1025 | 143/150 | 143/150 | 1e−66 |
| ref\|XP_051017.1\| | sapiens] | | (95%) | (95%) | |
| (XM_051017) | | | | | |
| gi\|13938170\| | (protein for | 1044 | 143/150 | 143/150 | 4e−66 |
| gb\|AAH07201.1\|AAH07201 | IMAGE: 2961284) [Homo | | (95%) | (95%) | |
| (BC007201) | sapiens] | | | | |
| gi\|18552587\|ref\|XP_ | (protein for | 180 | 50/58 | 51/58 | 4e−17 |
| 087161.1\| | IMAGE: 2961284) [Homo | | (86%) | (87%) | |
| (XM_087161) | sapiens] | | | | |
| gi\|9623317\|gb\|AAF90112.1\| | stretchin-MLCK | 281 | 35/105 | 50/105 | 7e−8 |
| AF254363_1 | [Drosophila | | (33%) | (47%) | |
| (AF254363) | melanogaster] | | | | |
| gi\|17559576\|ref\|NP_ | titin | 2783 | 39/120 | 57/120 | 1e−7 |
| 504582.1\| | [Caenorhabditis | | (32%) | (47%) | |
| (NM_072181) | elegans] | | | | |

Table 2D lists the domain descriptions from DOMAIN analysis results against NOV2. This indicates that the NOV sequence has properties similar to those of other proteins known to contain this domain.

TABLE 2D
Domain Analysis of NOV2

gnl|Smart|smart00409, IG, Immunoglobulin
CD-Length = 86 residues, 100.0% aligned
Score = 57.4 bits (137), Expect = 1e−09

Mac25 is a follistatin (FS)-like protein that has a growth-suppressing effect on a p53-deficient osteosarcoma cell line (Saos-2). The protein exhibits a strong homology to FS, an activin-binding protein, and part of its sequence includes the consensus sequence of the member of the Kazal serine protease inhibitor family. The mac25 protein was localized in the cytoplasm and secreted into culture medium (1). Addition of recombinant mac25 protein (10-7 M) into the culture medium induced significant suppression of the growth of human cervical carcinoma cells (HeLa) and murine embryonic carcinoma cells (P19), as well as osteosarcoma cells (Saos-2). The mac25 protein was co-immunoprecipitated with activin A, a result that suggests that mac25 may be a secreted tumor-suppressor that binds activin A. The mac25 exhibits homology to insulin-like growth factor-binding proteins (IGF-BPs) and to fibroblast growth factor receptor. The multi-functional nature of mac25 protein may be important for growth-suppression and/or cellular senescence.

The disclosed NOV2 nucleic acid of the invention encoding a Mac25/IGFBP7-like protein includes the nucleic acid whose sequence is provided in Table 2A or a fragment thereof. The invention also includes a mutant or variant nucleic acid any of whose bases may be changed from the corresponding base shown in Table 2A while still encoding a protein that maintains its Mac25/IGFBP7-like activities and physiological functions, or a fragment of Such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2B percent of the bases may be so changed.

The disclosed NOV2 protein of the invention includes the Mac25/IGFBP7-like protein whose sequence is provided in Table 2B. The invention also includes a mutant or variant protein any of whose residues may be changed from the corresponding residue shown in Table 2B while still encoding a protein that maintains its Mac25/IGFBP7-like activities and physiological functions, or a functional fragment thereof. In the mutant or variant protein, up to about 70 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Mac25/IGFBP7-like protein (NOV2) may function as a member of a “Mac25/IGFBP7 family”. Therefore, the NOV2 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV2 nucleic acids and proteins of the invention are useful in potential therapeutic applications implicated in diseases including but not limited to various pathologies and disorders as indicated below. For example, a cDNA encoding the Mac25/IGFBP7-like protein (NOV2) may be useful in gene therapy, and the Mac25/IGFBP7-like protein (NOV2) may be useful when administered to a subject in need thereof. By way of nonlimiting example, the compositions of the present invention will have efficacy for treatment of patients suffering from Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, fertility, hypogonadism, endometriosis, hemophilia, hypercoagulation, idiopathic thrombocytopenic purpura, immunodeficiencies, graft versus host disease, or other pathologies or conditions. The NOV2 nucleic acid encoding the Mac25/IGFBP7-like protein of the invention, or fragments thereof, may further be useful in diagnostic applications, wherein the presence or amount of the nucleic acid or the protein are to be assessed.

NOV2 nucleic acids and polypeptides are further useful in the generation of antibodies that bind immuno-specifically to the novel NOV2 substances for use in therapeutic or diagnostic methods. These antibodies may be generated according to methods known in the art, using prediction from hydrophobicity charts, as described in the “Anti-NOVX Antibodies” section below. The disclosed NOV2 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV3

A disclosed NOV3 nucleic acid of 1703 nucleotides (also referred to as CG57560-01) encoding a Calmodulin Binding Protein Kinase-like protein is shown in Table 3A. Putative untranslated regions upstream and/or downstream from the coding region, if any, are underlined, and the start and stop codons are in bold letters.

TABLE 3A
NOV3 nucleotide sequence.

(SEQ ID NO:5)

CCAGGTTGGGGTCTCCCAAAAGCAGCCCCTATGTTGTGGGGAAGTGGAGA
GTAGTACAGCTAAGCCAGACCCCATTGTGCCCGCAGGTTAGAGCCTGGCA
ATGCCGTTTGGGTGTGTGACTCTGGGCGACAAGAAGAACTATAACCAGCC
ATCGGAGGTGACTGACAGATATGATTTGGGACAGGTCATCAAGACGGAGG
AGTTTTGTGAAATCTTCCGGGCCAAGGACAAGACGACAGGCAAGCTGCAC
ACCTGCAAGAAGTTCCAGAAGCGGGACGGCCGCAAGGTGCGGAAAGCTGC
CAAGAACGAGATAGGCATCCTCAAGATGGTGAAGCATCCCAACATCCTAC
AGCTGGTGGATGTGTTTGTGACCCGCAAGGAGTACTTTATCTTCCTGGAG
CTGGCCACGGGGAGGGAGGTGTTTGACTGGATCCTGGACCAGGGCTACTA
CTCGGAGCGAGACACAAGCAACGTGGTACGGCAAGTCCTGGAGGCCGTGG
CCTATTTGCACTCACTCAAGATCGTGCACAGGAATCTCAAGCTGGAGAAC
CTGGTTTACTACAACCGGCTGAAGAACTCGAAGATTGTCATCAGTGACTT
CCATCTGGCTAAGCTAGAAAATGGCCTCATCAAGGAGCCCTGTGGGACCC
CCGAGTATCTGGCCCCAGAGGTGGTAGGCCGGCAGCGGTATGGACGCCCT
GTGGACTGCTGGGCCATTGGAGTCATCATGTACATCCTGCTTTCAGGCAA
CCCACCTTTCTATGAGGAGGTGGAAGAAGATGATTATGAGAACCATGATA
AGAATCTCTTCCGCAAGATCCTGGCTGGTGACTATGAGTTTGACTCTCCA
TATTGGGATGATATTTCGCAGGCAGCCAAAGACCTGGTCACAAGGCTGAT
GGAGGTGGAGCAAGACCAGCGGATCACTGCAGAAGAGGCCATCTCCCATG
AGTGGATTTCTGGCAATGCTGCTTCTGATAAGAACATCAAGGATGGTGTC
TGTGCCCAGATTGAAAAGAACTTTGCCAGGGCCAAGTGGAAGAAGGCTGT
CCGAGTGACCACCCTCATGAAACGGCTCCGGGCACCAGAGCAGTCCAGCA
CGGCTGCAGCCCAGTCGGCCTCAGCCACAGACACTGCCACCCCCGGGGCT
GCAGGTGGGGCCACAGCTGCAGCTGCGAGTGGAGCTACCTCAGCCCCTGA
GGGTGATGCTGCTCGTGCTGCAAAGAGTGATAATGTGGCCCCCGCAGACC
GTAGTGCCACCCCAGCCACAGATGGAAGTGCCACCCCAGCCACTGATGGC
AGTGTCACCCCAGCCACCGATGGAAGCATCACTCCAGCCACTGATGGGAG
TGTCACCCCAGCCACTGACAGGAGCGCTACTCCAGCCACTGATGGGAGAG
CCACACCAGCCACAGAAGAGAGCACTGTGCCCACCACCCAAAGCAGTGCC
ATGCTGGCCACCAAGGCAGCTGCCACCCCTGAGCCGGCTATGGCCCAGCC
GGACAGCACAGCCCCAGAGGGCGCCACAGGCCAGGCTCCACCCTCTAGTA
AAGGGGAAGAGGCTGCTGGTTATGCCCAGGAGTCTCAAAGGGAGGAGGCC
AGCTGAGTAGGCAGCCTGGTGAGGGGGGGCAGGGGATGGGCAGGAGGGTG
GGAGAGTGGATGAGGGGCTTCTCACTGTACATAGAGTCACTGGCATGAT
GCCC

In a search of public sequence databases, the NOV3 nucleic acid sequence, located on chromsome 3 has 1015 of 1158 bases (87%) identical to a gb:GENBANK-ID:RATCBVA|acc:L22557.1 mRNA from Rattus norvegicus (Rattus norvegicus vesicla-associate calmodulin-binding protein mRNA, complete cds). Public nucleotide databases include all GenBank databases and the GeneSeq patent database.

The disclosed NOV3 polypeptide (SEQ ID NO:6) encoded by SEQ ID NO:3 has 501 amino acid residues and is presented in Table 3B using the one-letter amino acid code. Signal P, Psort and/or Hydropathy results predict that NOV3 has no signal peptide and is likely to be localized in the in the cytoplasm with a certainty of 0.4500.

TABLE 3B
Encoded NOV3 protein sequence.

(SEQ ID NO:6)

MPFGCVTLGDKKNYNQPSEVTDRYDLGQVIKTEEFCEIFRAKDKTTGKLH
TCKKFQKRDCRKVRKAAKNEIGILKMVKHPNILQLVDVFVTRKEYFIFLE
LATGREVFDWILDQGYYSERDTSNVVRQVLEAVAYLHSLKIVHRNLKLEN
LVYYNRLKNSKIVISDFHLAKLENGLIKEPCGTPEYLAPEVVGRQRYGRP
VDCWAIGVIMYILLSGNPPFYEEVEEDDYENHDKNLFRKILAGDYEFDSP
YWDDISQAAKDLVTRLMEVEQDQRITAEEAISHEWISGNAASDKNIKDGV
CAQIEKNFARAKWKKAVRVTTLMKRLRAPEQSSTAAAQSASATDTATPGA
AGGATAAAASGATSAPEGDAARAAKSDNVAPADRSATPATDGSATPATDG
SVTPATDGSITPATDGSVTPATDRSATPATDGRATPATEESTVPTTQSSA
MLATKAAATPEPAMAQPDSTAPEGATGQAPPSSKGEEAAGYAQESQREEA
S

A search of sequence databases reveals that the NOV3 amino acid sequence has 1015 of 1158 amino acid residues (87%) identical to, and 1015 of 1158 amino acid residues (87%) similar to, the 3655 amino acid residue gb:GENBANK-ID:RATCBVA|acc:L22557.1 protein from Rattus norvegicus (Rattus norvegicus vesicla-associate calmodulin-binding protein mRNA, complete cds). Public amino acid databases include the GenBank databases, SwissProt, PDB and PIR.

NOV3 is expressed in at least Bone Marrow, Brain, Hypothalamus, Thalamus. This information was derived by determining the tissue sources of the sequences that were included in the invention including but not limited to SeqCalling sources, Public EST sources, Literature sources, and/or RACE sources.

The disclosed NOV3 polypeptide has homology to the amino acid sequences shown in the BLASTP data listed in Table 3C.

TABLE 3C
BLAST results for NOV3

| Gene Index/ | | Length | Identity | Positives | |
|---|---|---|---|---|---|
| Identifier | Protein/Organism | (aa) | (%) | (%) | Expect |
| gi\|16924331\|gb\|AAH17363.1\| | protein MGC8407 | 501 | 501/501 | 501/501 | 0.0 |
| AAH17363 | [Homo sapiens] | | (100%) | (100%) | |
| (BC017363) | | | | | |
| gi\|13129008\|ref\|NP_ | protein MGC8407 | 501 | 500/501 | 500/501 | 0.0 |
| 076951.1\| | [Homo sapiens] | | (99%) | (99%) | |
| (NM_024046) | | | | | |
| gi\|17160946\|gb\|AAH17634.1\| | Similar to vesicle- | 512 | 468/512 | 476/512 | 0.0 |
| AAH17634 | associated calmodulin- | | (91%) | (92%) | |
| (BC017634 | binding protein [Mus | | | | |
| | musculus] | | | | |
| gi\|13027458\|ref\|NP_ | vesicle- | 504 | 459/504 | 465/504 | 0.0 |
| 076490.1 | associated | | (91%) | (92%) | |
| (NM_024000) | calmodulin- | | | | |
| | binding protein | | | | |
| | [Rattus | | | | |
| | norvegicus] | | | | |
| gi\|13649042\|ref\|XP_ | protein MGC8407 | 478 | 469/471 | 470/471 | 0.0 |
| 017796.1\| | [Homo sapiens] | | (99%) | (99%) | |
| (XM_017796) | | | | | |

Table 3D lists the domain descriptions from DOMAIN analysis results against NOV3. This indicates that the NOV3 sequence has properties similar to those of other proteins known to contain this domain.

TABLE 3E
Domain Analysis of NOV

gnl|Smart|smart0022O, S_TKc, Serine/Threonine protein kinases,
catalytic domain; Phosphotransferases. Serine or threonine-specific
kinase subfamily.
CD-Length = 256 residues, 100.0% aligned
Score = 271 bits (693), Expect = 7e−74

Protein phosphorylation is a fundamental process for the regulation of cellular functions. The coordinated action of both protein kinases and phosphatases controls the levels of phosphorylation and, hence, the activity of specific target proteins. One of the predominant roles of protein phosphorylation is in signal transduction, where extracellular signals are amplified and propagated by a cascade of protein phosphorylation and dephosphorylation events. Eukaryotic protein kinases are enzymes that belong to a very extensive family of proteins which share a conserved catalytic core common with both serine/threonine and tyrosine protein kinases. There are a number of conserved regions in the catalytic domain of protein kinases. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme. Protein kinases are excellent small molecule drug targets for therapeutic intervention.

The disclosed NOV3 nucleic acid of the invention encoding a Calmodulin Binding Protein Kinase-like protein includes the nucleic acid whose sequence is provided in Table 3A or a fragment thereof. The invention also includes a mutant or variant nucleic acid any of whose bases may be changed from the corresponding base shown in Table 3A while still encoding a protein that maintains its Calmodulin Binding Protein Kinase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 13 percent of the bases may be so changed.

The disclosed NOV3 protein of the invention includes the Calmodulin Binding Protein Kinase-like protein whose sequence is provided in Table 3B. The invention also includes a mutant or variant protein any of whose residues may be changed from the corresponding residue shown in Table B while still encoding a protein that maintains its Calmodulin Binding Protein Kinase-like activities and physiological functions, or a functional fragment thereof. In the mutant or variant protein, up to about 13 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Calmodulin Binding Protein Kinase-like protein (NOV3) may function as a member of a “Calmodulin Binding Protein Kinase family”. Therefore, the NOV3 nucleic acids and proteins identified here may be useful in potential therapeutic applications implicated in (but not limited to) various pathologies and disorders as indicated below. T he potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV3 nucleic acids and proteins of the invention are useful in potential therapeutic applications implicated in diseases including but not limited to various pathologies and disorders as indicated below. For example, a cDNA encoding the Calmodulin Binding Protein Kinase-like protein (NOV3) may be useful in gene therapy, and the Calmodulin Binding Protein Kinase-like protein (NOV3) may be useful when administered to a subject in need thereof. By way of nonlimiting example, the compositions of the present invention will have efficacy for treatment of patients suffering from hemophilia, hypercoagulation, idiopathic thrombocytopenic purpura, autoimmune disease, allergies, immunodeficiencies, transplantation, graft versus host disease, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, or other pathologies or conditions. The NOV3 nucleic acid encoding the Calmodulin Binding Protein Kinase-like protein of the invention, or fragments thereof, may further be useful in diagnostic applications, wherein the presence or amount of the nucleic acid or the protein are to be assessed.

NOV3 nucleic acids and polypeptides are further useful in the generation of antibodies that bind immuno-specifically to the novel NOV3 substances for use in therapeutic or diagnostic methods. These antibodies may be generated according to methods known in the art, using prediction from hydrophobicity charts, as described in the “Anti-NOVX Antibodies” section below. The disclosed NOV3 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV4

NOV4 includes two TRANSIENT RECEPTOR POTENTIAL-RELATED PROTEIN-like proteins disclosed below. The disclosed sequences have been named NOV4a and NOV4b.

NOV4a

A disclosed NOV4a nucleic acid of 4877 nucleotides (also referred to as CG57547-01) encoding a TRANSIENT RECEPTOR POTENTIAL-RELATED PROTEIN-like protein is shown in Table 4A. Putative untranslated regions upstream and/or downstream from the coding region, if any, are underlined, and the start and stop codons are in bold letters.

TABLE 4A
NOV4 nucleotide sequence.

| | |
|---|---|
| AGTCCCCAGCCCCGTCGCCGGCGGAGGCGGGCGCGGGCGCGTTCCTGTGGCCAGTCACCC | (SEQ ID NO:7) |
| GGAGGAGTTGGTCGCACAATTATGAAAGACTCGGCTTCTGCTGCTAGCGCCGGAGCTGAG | |
| TTAGTCCTGAGAAGGTTTCCCTGGGCGTTCCTTGTCCGGCCTCTGCTGCCGCCTCCGGAG | |
| ACGCTTCCCGATAGATGGCTACAGGCCGCGGAGGAGGAGGAGGTGGAGTTGCTGCCCTTC | |
| CGGAGTCCGCCCCGTGAGGAGAATGTCCCAGAAATCCTGGATAGAAAGCACTTTGACCAA | |
| GAGGGAATGTGTATATATTATACCAAGTTCCAAGGACCCTCACAGGTGCCTTCCAGGATG | |
| TCAAATTTGTCAGCAACTCGTCAGGTGTTTTTGTGGTCGCTTGGTCAAGCAACATGCTTG | |
| TTTTACTGCAAGTCTTGCCATGAAATACTCAGATGTGAAATTGGGTGACCATTTTAATCA | |
| GGCAATAGAAGAATGGTCTGTGGAAAAGCATACAGAACAGAGCCCAACGGATGCTTATGG | |
| AGTCATAAATTTTCAAGGGGGTTCTCATTCCTACAGAGCTAAGTATGTGAGGCTATCATA | |
| TGACACCAAACCTGAAGTCATTCTGCAACTTCTGCTTAAAGAATCGCAAATGGAGTTACC | |
| CAAACTTGTTATCTCTGTACATGGGGGCATGCAGAAATTTGAGCTTCACCCACGAATCAA | |
| GCAGTTGCTTGGAAAAGGTCTTATTAAAGCTGCAGTTACAACTGGAGCCTGGATTTTAAC | |
| TGGAGGAGTAAACACAGGTGTGGCAAAACATGTTGGAGATGCCCTCAAAGAACATGCTTC | |
| CAGATCATCTCGAAAGATTTGCACTATCGGAATAGCTCCATGGGGAGTGATTGAAAACAG | |
| AAATGATCTTGTTGGGAGAGATGTAGTTGCTCCTTATCAAACCTTATTGAACCCCCTGAG | |
| CAAATTGAATGTTTTGAATAATCTGCATTCCCATTTCATATTGGTGGATGATGGCACTGT | |
| TGGAAAGTATGGGGCGGAAGTCAGACTGAGAAGAGAACTTGAAAAAACTATTAATCAGCA | |
| AAGAATTCATGCTATTGGCCAGGGTGTCCCTGTGGTGGCACTTATATTTGAGGGTGGGCC | |
| AAATGTTATCCTCACAGTTCTTGAATACCTTCAGGAAAGCCCCCCTGTTCCAGTAGTTGT | |
| GTGTGAAGGAACAGGCAGAGCTGCAGATCTGCTAGCGTATATTCATAAACAAACAGAAGA | |
| AGGAGGGAATCTTCCTGATGCAGCAGAGCCCGATATTATTTCCACTATCAAAAAAACATT | |
| TAACTTTGGCCAGAATGAAGCACTTCATTTATTTCAAACACTGATGGAGTGCATGAAAAG | |
| AAAGGAGCTTATCACTGTTTTCCATATTGGGTCAGATGAACATCAAGATATAGATGTAGC | |
| AATACTTACTGCACTGCTAAAAGGTACTAATGCATCTGCATTTGACCAGCTTATCCTTAC | |
| ATTGGCATGGGATAGAGTTGACATTGCCAAAAATCATGTATTTGTTTATGGACAGCAGTG | |
| GCTGGTAGGATCCTTGGAACAAGCTATGCTTGATGCTCTTGTAATGGATAGAGTTGCATT | |
| TGTAAAACTTCTTATTGAAAATGGAGTAAGCATGCATAAATTCCTTACCATTCCGAGACT | |
| GGAAGAACTTTACAACACTAAACAAGGTCCAACTAATCCAATGCTGTTTCATCTTGTTCG | |
| AGACGTCAAACAGGGAAATCTTCCTCCAGGATATAAGATCACTCTGATTGATATAGGACT | |
| TGTTATTGAATATCTCATGGGAGGAACCTACAGATGCACCTATACTAGGAAACGTTTTCG | |
| ATTAATATATAATAGTCTTGGTGGAAATAATCGGAGGTCTGGCCGAAATACCTCCAGCAG | |
| CACTCCTCAGTTGCGAAAGAGTCATGAATCTTTTGGCAATAGGGCAGATAAAAAGGAAAA | |
| AATGAGGCATAACCATTTCATTAAGACAGCACAGCCCTACCGACCAAAGGTAGATACAGT | |
| TATGGAAGAAGGAAAGAAGAAAAGAACCAAAGATGAAATTGTAGACATTGATGATCCAGA | |
| AACCAAGCGCTTTCCTTATCCACTTAATGAACTTTTAATTTGGGCTTGCCTTATGAAGAG | |
| GCAGGTCATGGCCCGTTTTTTATGGCAACATGGTGAAGAATCAATGGCTAAAGCATTAGT | |
| TGCCTGTAAGATCTATCGTTCAATGGCATATGAAGCAAAGCAGAGTGACCTGGTAGATGA | |
| TACTTCAGAAGAACTAAAACAGTATTCCAGTGATTTTGGTCAGTTGGCCGTTGAATTATT | |
| AGAACAGTCCTTCAGACAAGATGAAACCATGGCTATGAAATTGCTCACTTATGAACTGAA | |
| GAACTGGAGTAATTCAACCTGCCTTAAGTTAGCAGTTTCTTCAAGACTTAGACCTTTTGT | |
| AGCTCACACCTGTACACAAATGTTGTTATCTGATATGTGGATGGGAAGGCTGAATATGAG | |
| GAAAAATTCCTGGTACAAGGTAATACTAAGCATTTTAGTTCCACCTGCCATATTGCTGTT | |
| AGAGTATAAAACTAAGGCTGAAATGTCCCATATCCCACAATCTCAAGATGCTCATCAGAT | |
| GACAATGGATGACAGCGAAAACAACTTTCAGAACATAACAGAAGAGATCCCCATGGAAGT | |
| GTTTAAAGAAGTACGGATTTTGGATAGTAATGAAGGAAAGAATGAGATGGAGATACAAAT | |
| GAAATCAAAAAAGCTTCCAATTACGCGAAAGTTTTATGCCTTTTATCATGCACCAATTGT | |
| AAAATTCTGGTTTAACACGTTGGCATATTTAGGATTTCTGATGCTTTATACATTTGTGGT | |
| TCTTGTACAAATGGAACAGTTACCTTCAGTTCAAGAATGGATTGTTATTGCTTATATTTT | |
| TACTTATGCCATTGAGAAAGTCCGTGAGGTATTTATGTCTGAAGCTGGGAAAGTAAACCA | |
| GAAGATTAAAGTATGGTTTAGTGATTACTTCAACATCAGTGATACAATTGCCATAATTTC | |
| TTTCTTCATTGGATTTGGACTAAGATTTGGAGCAAAATGGAACTTTGCAAATGCATATGA | |
| TAATCATGTTTTTGTGGCTGGAAGATTAATTTACTGTCTTAACATAATATTTTGGTATGT | |
| GCGTTTGCTAGATTTTCTAGCTGTAAATCAACAGGCAGGACCTTATGTAATGATGATTGG | |
| AAAAATGGTGGCCAATATGTTCTACATTGTAGTGATTATGGCTCTTGTATTACTTAGTTT | |
| TGGTGTTCCCAGAAAGGCAATACTTTATCCTCATGAAGCACCATCTTGGACTCTTGCTAA | |
| AGATATAGTTTTTCACCCATACTGGATGATTTTTGGTGAAGTTTATGCATACGAAATTGA | |
| TGTGTGTGCAAATGATTCTGTTATCCCTCAAATCTGTGGTCCTGGGACGTGGTTGACTCC | |
| ATTTCTTCAAGCAGTCTACCTCTTTGTACAGTATATCATTATGGTTAATCTTCTTATTGC | |
| ATTTTTCAGCAATGTGTATTTACAAGTGAAGGCAATTTCCAATATTGTATGGAAGTACCA | |
| GCGTTATCATTTTATTATGGCTTATCATGAGAAACCAGTTCTGCCTCCTCCACTTATCAT | |
| TCTTAGCCATATAGTTTCTCTGTTTTGCTGCATATGTAAGAGAAGAAAGAAAGATAAGAC | |
| TTCCGATGGACCAGAACTTTTCTTAACAGAAGAAGATCAAAAGAAACTTCATGATTTTGA | |
| AGAGCAGTGTGTTGAAATGTATTTCAATGAAAAAGATGACAAATTTCATTCTGGGAGTGA | |
| AGAGAGAATTCGTGTCACTTTTGAAAGAGTGGAACAGATGTGCATTCAGATTAAAGAAGT | |
| TGGAGATCGTGTCAACTACATAAAAAGATCATTACAATCATTAGATTCTCAAATTGGCCA | |
| TTTGCAAGATCTTTCAGCCCTGACGGTAGATACATTAAAAACACTCACTGCCCAGAAAGC | |
| GTCGGAAGCTAGCAAAGTTCATAATGAAATCACACGAGAACTGAGCATTTCCAAACACTT | |
| GGCTCAAAACCTTATTGATGATGGTCCTGTAAGACCTTCTGTATGGAAAAAGCATGGTGT | |
| TGTAAATACACTTAGCTCCTCTCTTCCTCAAGGTGATCTTGAAAGTAATAATCCTTTTCA | |
| TTGTAATATTTTAATGAAAGATGACAAAGATCCCCAGTGTAATATATTTGGTCAAGACTT | |
| ACCTGCAGTACCCCAGAGAAAAGAATTTAATTTTCCAGAGGCTGGTTCCTCTTCTGGTGC | |
| CTTATTCCCAAGTGCTGTTTCCCCTCCAGAACTGCGACAGAGACTACATGGGGTAGAACT | |
| CTTAAAAATATTTAATAAAAATCAAAAATTAGGCAGTTCATCTACTAGCATACCACATCT | |
| GTCATCCCCACCAACCAAATTTTTTGTTAGTACACCATCTCAGCCAAGTTGCAAAAGCCA | |
| CTTGGAAACTGGAACCAAAGATCAAGAAACTGTTTGCTCTAAAGCTACAGAAGGAGATAA | |
| TACAGAATTTGGAGCATTTGTAGGTCACAGAGATAGCATGGATTTACAGAGGTTTAAAGA | |
| AACATCAAACAAGATAAAATTGCAGAATAACAATACTTCTGAAAACACTTTGAAACGAGT | |
| GAGTTCTCTTGCTGGATTTACTGACTGTCACAGAACTTCCATTCCTGTTCATTCAAAACA | |
| AGCAGAAAAAATCAGTAGAAGGCCATCTACCGAAGACACTCATGAAGTAGATTCCAAAGC | |
| AGCTTTATTACTGAAGGATTGGTTACAAGATAGACCATCAAACAGAGAAATGGGTCTCAC | |
| TTCTCCATTTAAGCCAGCTATGGATACAAATTACTATTATTCAGCTGTGGAAAGAAATAA | |
| CTTGATGAGGTTATCACAGAGCATTCCATTTACACCTGTGCCTCCAAGAGGGGAGCCTGT | |
| CACAGTGTATCGTTTGGAAGAGAGTTCACCCAACATACTAAATAACAGCATGTCTTCTTG | |
| GTCACAACTAGGCCTCTGTGCCAAAATAGAGTTTTTAAGCAAAGAGGAGATGGGAGGAGG | |
| TTTACGAAGAGCTGTCAAAGTACAGTGTACCTGGTCAGAACATGATATCCTCAAATCAGG | |
| GCATCTTTATATTATCAAATCTTTTCTTCCAGAGGTGGTTAATACATGGTCAAGTATTTA | |
| CAAAGAAGATACAGTTCTGCATCTCTGTCTGAGAGAAATTCAACAACAGAGAGCAGCACA | |
| AAAGCTTACGTTTGCCTTTAATCAAATGAAACCCAAATCCATACCATATTCTCCAAGGTT | |
| CCTTGAAGTTTTCCTGCTGTATTGCCATTCAGCAGGACAGTGGTTTGCTGTGGAAGAATG | |
| TATGACTGGAGAATTTAGAAAATACAACAATAATAATGGAGATGAGATTATTCCAACTAA | |
| TACTCTGGAAGAGATCATGCTAGCCTTTAGCCACTGGACTTACGAATATACAAGAGGGGA | |
| GTTACTGGTACTTGATTTGCAAGGTGTTGGTGAAAATTTGACTGACCCATCTGTGATAAA | |
| AGCAGAAGAAAAGAGATCCTGTGATATGGTTTTTGGCCCAGCAAATCTAGGAGAAGATGC | |
| AATTAAAAACTTCAGAGCAAAACATCACTGTAATTCTTGCTGTAGAAAGCTTAAACTTCC | |
| AGATCTGAAGAGGAATGATTATACGCCTGATAAAATTATATTTCCTCAGGATGAGCCTTC | |
| AGATTTGAATCTTCAGCCTGGAAATTCCACCAAAGAATCAGAATCAACTAATTCTGTTCG | |
| TCTGATGTTATAATATTAATATTACTGAATCATTGGTTTTGCCTGCACCTCACAGAA | |

In a search of public sequence databases, the NOV4a nucleic acid sequence, located on chromsome 15 has 4374 of 4825 bases (90%) identical to a gb:GENBANK-ID:AF149013|acc:AF149013.1 mRNA from Mus musculus (Mus musculus transient receptor potential-related protein (ChaK) mRNA, complete cds). Public nucleotide databases include all GenBank databases and the GeneSeq patent database.

The disclosed NOV4a polypeptide (SEQ ID NO:8) encoded by SEQ ID NO:7 has 1856 amino acid residues and is presented in Table 4B using the one-letter amino acid code. Signal P, Psort and/or Hydropathy results predict that NOV4a has no signal peptide and is likely to be localized at the plasma membrane with a certainty of 0.6000.

TABLE 4B
Encoded NOV4a protein sequence.

(SEQ ID NO:8)

MSQKSWIESTLTKRECVYIIPSSKDPHRCLPGCQICQQLVRCFCGRLVKQ
HACFTASLAMKYSDVKLGDHFNQAIEEWSVEKHTEQSPTDAYGVINFQGG
SHSYRAKYVRLSYDTKPEVILQLLLKEWQMELPKLVISVHGGMQKFELHP
RIKQLLGKGLTKAAVTTGAWILTGGVNTGVAKHVGDALKEHASRSSRKIC
TIGIAPWGVIENRNDLVGRDVVAPYQTLLNPLSKLNVLNNLHSHFILVDD
GTVGKYGAEVRLRRELEKTINQQRIHAIGQGVPVVALIFEGGPNVILTVL
EYLQESPPVPVVVCEGTGRAADLLAYIHKQTEEGGNLPDAAEPDIISTIK
KTFNFGQNEALHLFQTLMECMKRKELITVFHIGSDEHQDIDVAILTALLK
GTNASAFDQLILTLAWDRVDIAKNHVFVYGQQWLVGSLEQAMLDALVMDR
VAFVKLLIENGVSMHKFLTIPRLEELYNTKQGPTNPNLFHLVRDVKQGNL
PPGYKTTLIDIGLVIEYLMGGTYRCTYTRKRFRLIYNSLGGNNRRSGRNT
SSSTPQLRKSHESFGNRADKKEKMRHNHFIKTAQPYRPKVDTVMEEGKKK
RTKDEIVDIDDPETKRFPYPLNELLIWACLMKRQVMARFLWQHGEESMAK
ALVACKIYRSMAYEAKQSDLVDDTSEELKQYSSDFGQLAVELLEQSFRQD
ETMAMKLLTYELKWWSNSTCLKLAVSSRLRPFVAHTCTQMLLSDMWMGRL
NMRKNSWYKVILSILVPPAILLLEYKTKAEMSHIPQSQDAHQMTMDDSEN
NFQNITEEIPMEVFKEVRILDSNEGKNEMEIQMKSKKLPITRKFYAFYHA
PIVKFWFNTLAYLGFLMLYTFVVLVQMEQLPSVQEWIVIAYIFTYAIEKV
REVFMSEAGKVNQKIKVWFSDYFNISDTIAIISFFIGFGLRFGAKWNFAN
AYDNHVFVAGRLIYCLNIIFWYVRLLDFLAVNQQAGPYVMMIGKMVANMF
YIVVIMALVLLSFGVPRKAILYPHEAPSWTLAKDIVFHPYWMIFGEVYAY
EIDVCANDSVIPQICGPGTWLTPFLQAVYLFVQYIIMVNLLIAFFSNVYL
QVKAISNIVWKYQRYHFIMAYHEKPVLPPPLIILSHIVSLFCCICKRRKK
DKTSDGPELFLTEEDQKKLHDFEEQCVEMYFNEKDDKFHSGSEERIRVTF
ERVEQMCIQIKEVGDRVNYIKRSLQSLDSQIGHLQDLSALTVDTLKTLTA
QKASEASKVHNEITRELSISKHLAQNLIDDGPVRPSVWKKHGVVNTLSSS
LPQGDLESNNPFHCNILMKDDKDPQCNIFGQDLPAVPQRKEFNFPEAGSS
SGALFPSAVSPPELRQRLHGVELLKIFNKNQKLGSSSTSIPHLSSPPTKF
FVSTPSQPSCKSHLETGTKDQETVCSKATEGDNTEFGAFVGHRDSMDLQR
FKETSNKIKLQNNNTSENTLKRVSSLAGFTDCHRTSIPVHSKQAEKISRR
PSTEDTHEVDSKAALLLKDWLQDRPSNREMGLTSPFKPAMDTNYYYSAVE
RNNLMRLSQSIPFTPVPPRGEPVTVYRLEESSPNILNNSMSSWSQLGLCA
KIEFLSKEEMGGGLRRAVKVQCTWSEHDILKSGHLYIIKSFLPEVVNTWS
SIYKEDTVLHLCLREIQQQRAAQKLTFAFNQMKPKSIPYSPRFLEVFLLY
CHSAGQWFAVEECMTGEFRKYNNNNGDEIIPTNTLEEIMLAFSHWTYETT
RGELLVLDLQGVGENLTDPSVIKAEEKRSCDMVFGPANLGEDAIKNFRAK
HHCNSCCRKLKLPDLKRNDYTPDKIIFPQDEPSDLNLQPGNSTKESESTN
SVRLML

A search of sequence databases reveals that the NOV4a amino acid sequence has 1747 of 1863 amino acid residues (93%) identical to, and 1803 of 1863 amino acid residues (96%) similar to the 1863 amino acid residue ptnr:SPTREMBL-ACC:Q9JLQ1 protein from Mus musculus (Mouse) (TRANSIENT RECEPTOR POTENTIAL-RELATED PROTEIN). Public amino acid databases include the GenBank databases, SwissProt, PDB and PIR.

NOV4a is expressed in at least Adrenal Gland/Suprarenal gland, Bone Marrow, Brain, Bronchus, Cartilage, Colon, Hippocampus, Kidney, Liver, Lymph node, Skeletal Muscle, Stomach, Substantia Nigra, Tonsils and Whole Organism. This information was derived by determining the tissue sources of the sequences that were included in the invention including but not limited to SeqCalling sources, Public EST sources, Literature sources, and/or RACE Sources.

NOV4b

A disclosed NOV4b nucleic acid of 5626 nucleotides (also referred to as CG57547-02) encoding a TRANSIENT RECEPTOR POTENTIAL-RELATED PROTEIN-like protein is shown in Table 4C. Putative untranslated regions upstream and/or downstream from the coding region, if any, are underlined, and the start and stop codons are in bold letters.

TABLE 4C
NOV4b nucleotide sequence.

| | |
|---|---|
| AGTCCCCAGCCCCGTCGCCGGCGGAGGCGGGCGCGGGCGCGTTCCTGTGGCCAGTCACCC | (SEQ ID NO:9) |
| GGAGGAGTTGGTCGCACAATTATGAAAGACTCGGCTTCTGCTGCTAGCGCCGGAGCTGAG | |
| TTAGTCCTGAGAAGGTTTCCCTGGGCGTTCCTTGTCCGGCCTCTGCTGCCGCCTCCGGAG | |
| ACGCTTCCCGATAGATGGCTACAGGCCGCGGAGGAGGAGGAGGTGGAGTTGCTGCCCTTC | |
| CGGAGTCCGCCCCGTGAGGAGAATGTCCCAGAAATCCTGGATAGAAAGCACTTTGACCAA | |
| GAGGGAATGTGTATATATTATACCAAGTTCCAAGGACCCTCACAGGTGCCTTCCAGGATG | |
| TCAAATTTGTCAGCAACTCGTCAGGGTTTTTGTGGTCGCTTGGTCAAGCAACATGCTTGT | |
| TTTACTGCAAGTCTTGCCATGAAATACTCAGATGTGAAATTGGGTGACCATTTTAATCAG | |
| GCAATAGAAGAATGGTCTGTGGAAAAGCATACAGAACAGAGCCCAACGGATGCTTATGGA | |
| GTCATAAATTTTCAAGGGGGTTCTCATTCCTACAGAGCTAAGTATGTGAGGCTATCATAT | |
| GACACCAAACCTGAAGTCATTCTGCAACTTCTGCTTAAAGAATGGCAAATGGAGTTACCC | |
| AAACTTGTTATCTCTGTACATGGGGGCATGCAGAAATTTGAGCTTCACCCACGAATCAAG | |
| CAGTTGCTTGGAAAAGGTCTTATTAAAGCTGCAGTTACAACTGGAGCCTGGATTTTAACT | |
| GGAGGAGTAAACACAGGTACAGGTGTGGCAAAACATGTTGGAGATGCCCTCAAAGAACAT | |
| GCTTCCAGATCATCTCGAAAGATTTGCACTATCGGAATAGCTCCATGGGGAGTGATTGAA | |
| AACAGAAATGATCTTGTTGGGAGAGATGTAAGAATTATTTATCAAACCTTATTGAACCCC | |
| CTGAGCAAATTGAATGTTTTGAATAATCTGCATTCCCATTTCATATTGGTGGATGATGGC | |
| ACTGTTGGAAAGTATGGGGCGGAAGTCAGACTGAGAAGAGAACTTGAAAAAACTATTAAT | |
| CAGCAAAGAATTCATATTGGCCAGGGTGTCCCTGTGGTGGCACTTATATTTGAGGGTGGG | |
| CCAAATGTTATCCTCACAGTTCTTGAATACCTTCAGGAAAGCCCCCCTGTTCCAGTAGTT | |
| GTGTGTGAAGGAACAGGCAGAGCTGCAGATCTGCTAGCGTATATTCATAAACAAACAGAA | |
| GAAGGAGGGGATGCAGCAGAGCCCGATATTATTTCCACTATCAAAAAAACATTTAACTTT | |
| GGCCAGAATGAAGCACTTCATTTATTTCAAACACTGATGGAGTGCATGAAAAGAAAGGAG | |
| CTTGTAACTGTTTTCCATATTGGGTCAGATGAACATCAAGATATAGATGTAGCAATACTT | |
| ACTGCACTGCTAAAAGGTACTAATGCATCTGCATTTGACCAGCTTATCCTTACATTGGCA | |
| TGGGATAGAGTTGACATTGCCAAAAATCATGTATTTGTTTATGGACAGCAGTGGCCATTG | |
| CACTCCAGCCTGGGCAACAGAGTGAGACTCTCTCTCAAAAAAAAAAAACAAAAACAAAAA | |
| CAAAAACAAAAACAAAAACCAACACCTAGAAATTCAGAGTTAGTTGGATCCTTGGAACAA | |
| GCTATGCTTGATGCTCTTGTAATGGATAGAGTTGCATTTGTAAAACTTCTTATTGAAAAT | |
| GGAGTAAGCATGCATAAATTCCTTACCATTCCGAGACTGGAAGAACTTTACAACACTAAT | |
| CTTCCTCCAGGATATAAGATCACTCTGATTGATATAGGACTTGTTATTGAATATCTCATG | |
| GGAGGAACCTACAGATGCACCTATACTAGGAAACGTTTTCGATTAATATATAATAGTCTT | |
| GGTGGAAATAATCGGTTTTCCTTCCAGGAGCCCAACCACACTCGCACGGTAAATATTAGA | |
| GACAAATCTCCTCATGCTTCTGGCAAGAAGAAGGGAAACAAGAAAAGAACCAAAGATGAA | |
| ATTGTAGACATTGATGATCCAGAAACCAAGCGCTTTCCTTATCCACTTAATGAACTTTTA | |
| ATTTGGGCTTGCCTTATGAAGAGGCAGGTCATGGCCCGTTTTTTATGGCAACATGGTGAA | |
| GAATCAATGGCTAAAGCATTAGTTGCCTGTAAGATCTATCGTTCAATGGCATATGAAGCA | |
| AAGCAGAGTGACCTGGTAGATGATACTTCAGAAGAACTAAAACAGTATTCCAAGGATTTT | |
| GGTCAGTTGGCCGTTGAATTATTAGAACAGTCCTTCAGACAAGATGAAACCATGGCTATG | |
| AAATTGCTCACTTATGAACTGAAGAACTGGAGTAATTCAACCTGCCTTAAGTTAGCAGTT | |
| TCTTCAAGACTTAGACCTTTTGTAGCTCACACCTGTACACAAATGTTGTTATCTGATATG | |
| TGGATGGGAAGGCTGAATATGAGGAAAAATTCCTGGTACAAGGTAATACTAAGCATTTTA | |
| GTTCCACCTGCCATATTGCTGTTAGAGTATAAAACTAAGGCTGAAATGTCCCATATCCCA | |
| CAATCTCAAGATGCTCATCAGATGACAATGGATGACAGCGAAAACAACAGTAATGAAGGA | |
| AAGAATGAGATGGAGATACAAATGAAATCAAAAAAGCTTCCAATTACGCGAAAGTTTTAT | |
| GCCTTTTATCATGCACCAATTGTAAAATTCTGGTTTAACACGTTGGCATATTTAGGATTT | |
| CTGATGCTTTATACATTTGTGGTTCTTGTACAAATGGAACAGTTACCTTCAGTTCAAGAA | |
| TGGATTGTTATTGCTTATATTTTTACTTATGCCATTGAGAAAGTCCGTGAGATCTTTATG | |
| TCTGAAGCTGGGAAAGTAAACCAGAAGATTAAAGTATGGTTTAGTGATTACTTCAACATC | |
| AGTGATACAATTGCCATAATTTCTTTCTTCATTGGATTTGGACTAAGATTTGGAGCAAAA | |
| TGGAACTTTGCAAATGCATATGATAATCATGTTTTTGTGGCTGGAAGATTAATTTACTGT | |
| CTTAACATAATATTTTGGTATGTGCGTTTGCTAGATTTTCTAGCTGTTAATCAACAGGCA | |
| GGACCTTATGTAATGATGATTGGAAAAATGGTAAATATGTTCTACATTGTAGTGATTATG | |
| GCTCTTGTATTACTTAGTTTTGGTGTTCCCAGAAAGGCAATACTTTATCCTCATGAAGCA | |
| CCATCTTGGACTCTTGCTAAAGATATAGTTTTTCACCCATACTGGATGATTTTTGGTGAA | |
| GTTTATGCATACGAAATTGATTGTGGTCCTGGGACGTGGTTGACTCCATTTCTTCAAGCA | |
| GTCTACCTCTTTGTACAGTATATCATTATGGTTAATCTTCTTATTGCATTTTTCAAGAGC | |
| AATGTGTATTTACAAGTGAAGGCAATTTCCAATATTGTATGGAAGTACCAGCGTTATCAT | |
| TTTATTATGGCTTATCATGAGAAACCAGTTCTGCCTCCTCCACTTATCATTCTTAGCCAT | |
| ATAGTTTCTCTGTTTTGCTGCATATGTAAGAGAAGAAAGAAAGATAAGACTTCCGATGGA | |
| CCAAGTAAGATAGAACTTTTCTTAACAGAAGAAGATCAAAAGAAACTTCATGATTTTGAA | |
| GAGCAGTGTGTTGAAATGTATTTCAATGAAAAAGATGACAAATTTCATTCTGGGAGTGAA | |
| GAGAGAATTCGTGTCACTTTTGAAAGAGTGGAACAGAAGCCCATTCAGATTAAAGAAGTT | |
| GGAGATCGTGTCAACTACATAAPAAGATCATTACAATCATTAGATTCTCAAATTGGCCAT | |
| TTGCAAGATCTTTCAGCCCTGACGGTAGATACATTAAAAACACTCACTGCCCAGAAAGCG | |
| TCGGAAGCTAGCAAAGTTCATAATGAAATCACACGAGAACTGAGCATTTCCAAACACTTG | |
| GCTCAAAACCTTATTGATGATGGTCCTGTAAGACCTTCTGTATGGAAAAAGCATGGTGTT | |
| GTAAATACACTTAGCTCCTCTCTTCCTCAAGGTGATCTTGAAAGTAATAATCCTTTTCAT | |
| TGTAATATTTTAATGAAAGATGACAAAGATCCCCAGTGTAATATATTTGGTCAAGACTTA | |
| CCTGCAGTACCCCAGAGAAAAGAATTTAATTTTCCAGAGGCTGGTTCCTCTTCTGGTGCC | |
| TTATTCCCAAGTGCTGTTTCCCCTCCAGAACTGCGACAGAGACTACATGGGGTAGAACTC | |
| TTAAAAATATTTAATAAAAATCAAAAATTAGGCAGTTCATCTACTAGCATACCACATCTG | |
| TCATCCCCACCAACCAAATTTTTTGTTAGTACACCATCTCAGCCAAGTTGCAAAAGCCAC | |
| TTGGAAACTGGAACCAAAGATCAAGAAACTGTTTGCTCTAAAGCTACAGAAGGAGATAAT | |
| ACAGAATTTGGAGCATTTGTAGGTCACAGAGATAGCATGGATTTACAGAGGTTTAAAGAA | |
| ACATCAAACAAGATAAAATTGCAGAATAACAATACTTCTGAAAACACTTTGAAACGAGTG | |
| AGTTCTCTTGCTGGATTTACTGACTGTCACAGAACTTCCATTCCTGTTCATTCAAAACAA | |
| GCAGAAAAAATCAGTAGAAGGCCATCTACCGAAGACACTCATGAAGTAGATTCCAAAGCA | |
| GCTTTAATACCGGATTGGTTACAAGATAGACCATCAAACAGAGAAATGGGTCTCACTTCT | |
| CCATTTAAGCCAGCTATGGATACAAATTACTATTATTCAGCTGTGGAAAGAAATAACTTG | |
| ATGAGGTTATCACAGAGCATTCCATTTACACCTGTGCCTCCAAGAGGGGAGCCTGTCACA | |
| GTGTATCGTTTGGAAGAGAGTTCACCCAACATACTAAATAACAGCATGTCTTCTTGGTCA | |
| CAACTAGGCCTCTGTGCCAAAATAGAGTTTTTAAGCAAAGAGGAGATGGGAGGAGGTTTA | |
| CGAAGAGCTGTCAAAGTACAGTGTACCTGGTCAGAACATGATATCCTCAAATCAGGGCAT | |
| CTTTATATTATCAAATCTTTTCTTCCAGAGGTGGTTAATACATGGTCAAGTATTTACAAA | |
| GAAGATACAGTTCTGCATCTCTGTCTGAGAGAAATTCAACAACAGAGAGCAGCACAAAAG | |
| CTTACGTTTGCCTTTAATCAAATGAAACCCAAATCCATACCATATTCTCCAGGGGAGTTA | |
| CTGGTACTTGATTTGCAAGGTGTTGGTGAAAATTTGACTGACCCATCTGTGATAAAAGCA | |
| GAAGAAAAGAGATCCTGTGATATGGTTTTTGGCCCAGCAAATCTAGGAGAAGATGCAATT | |
| AAAAACTTCAGAGCAAAACATCACTGTAATTCTTGCTGTAGAAAGCTTAAACTTCCAGAT | |
| CTGAAGAGGAATGATTATACGCCTGATAAAATTATATTTCCTCAGGATGAGCCTTCAGAT | |
| TTGAATCTTCAGCCTGGAAATTCCACCAAAGAATCAGAATCAACTAATTCTGTTCGTCTG | |
| ATGTTATAATATTAATATTACTGAATCATTGGTTTTGCCTGCACCTCACAGAAATGTTAC | |
| TGTGTCACTTTTCCCTCGGGAGGAAATTGTTTGGTAATATAGAAAG | |

In a search of public sequence databases, the NOV4b nucleic acid sequence, located on chromsome 15 has 1134 of 1246 bases (91%) identical to a gb:GENBANK-ID:AF149013|acc:AF149013.1 mRNA from Mus musculus (Mus musculus transient receptor potential-related protein (ChaK) mRNA, complete cds). Public nucleotide databases include all GenBank databases and the GeneSeq patent database.

The disclosed NOV4b polypeptide (SEQ ID NO: 10) encoded by SEQ ID NO:9 has 1815 amino acid residues and is presented in Table 4D using the one-letter amino acid code. Signal P, Psort and/or Hydropathy results predict that NOV4b has no signal peptide and is likely to be localized at the plasma membrane with a certainty of 0.6000.

TABLE 4D
Encoded NOV4b protein sequence.

(SEQ ID NO:10)

MKDSASAASAGAELVLRRFPWAFLVRPLLPPPETLPDRWLQAAEEEEVEL
LPFRSPPREENVPEILDRKHFDQEGMCIYYTKFQGPSQVPSRMSNLSATR
QGFCGRLVKQHACFTASLANKYSDVKLGDHFNQAIEEWSVEKHTEQSPTD
AYGVINFQGGSHSYRAKYVRLSYDTKPEVILQLLLKEWQMELPKLVISVH
GGMQKFELHPRIKQLLGKGLIKAAVTTGAWILTGGVNTGTGVAKHVGDAL
KEHASRSSRKICTIGIAPWGVIENRNDLVGRDVRIIYQTLLNPLSKLNVL
NNLHSHFILVDDGTVGKYGAEVRLRRELEKTINQQRIHIGQGVPVVALIF
EGGPNVILTVLEYLQESPPVPVVVCEGTGRAADLLAYIHKQTEEGGDAAE
PDIISTIKKTFNFGQNEALHLFQTLMECMKRKELVTVFHIGSDEHQDIDV
AILTALLKGTNASAFDQLILTLAWDRVDIAKNHVFVYGQQWPLHSSLGNR
VRLSLKKKKQKQKQKQKQKPTPRNSELVGSLEQAMLDALVMDRVAFVKLL
IENGVSMHKFLTIPRLEELYNTNLPPGYKITLIDIGLVIEYLMGGTYRCT
YTRKRFRLIYNSLGGNNRFSFQEPNHTRTVNIRDKSPHASGKKKGKKKRT
KDEIVDIDDPETKRFPYPLNELLIWACLMKRQVMARFLWQHGEESMAKAL
VACKIYRSMAYEAKQSDLVDDTSEELKQYSKDFGQLAVELLEQSFRQDET
MANKLLTYELKNWSNSTCLKLAVSSRLRPFVAHTCTQMLLSDMWMGRLNM
RKNSWYKVILSILVPPAILLLEYKTKAEMSHIPQSQDAHQMTMDDSENNS
NEGKNEMEIQMKSKKLPITRKFYAFYHAPIVKFWFNTLAYLGFLMLYTFV
VLVQMEQLPSVQEWIVIAYIFTYAIEKVREIFMSEAGKVNQKIKVWFSDY
FNISDTIAIISFFIGFGLRFGAKWNFANAYDNHVFVAGRLIYCLNIIFWY
VRLLDFLAVNQQAGPYVMMIGKMVNMFYIVVIMALVLLSFGVPRKAILYP
HEAPSWTLAKDIVFHPYWMIFGEVYAYEIDCGPGTWLTPFLQAVYLFVQY
IIMVNLLIAFFKSNVYLQVKAISNIVWKYQRYHFIMAYHEKPVLPPPLII
LSHIVSLFCCICKRRKKDKTSDGPSKIELFLTEEDQKKLHDFEEQCVEMY
FNEKDDKFHSGSEERIRVTFERVEQKPIQIKEVGDRVNYIKRSLQSLDSQ
IGHLQDLSALTVDTLKTLTAQKASEASKVHNEITRELSISKHLAQNLIDD
GPVRPSVWKKHGVVNTLSSSLPQGDLESNNPFHCNILMKDDKDPQCNIFG
QDLPAVPQRKEFNFPEAGSSSGALFPSAVSPPELRQRLHGVELLKIFNKN
QKLGSSSTSIPHLSSPPTKFFVSTPSQPSCKSHLETGTKDQETVCSKATE
GDNTEFGAFVGHRDSMDLQRFKETSNKIKLQNNNTSENTLKRVSSLAGFT
DCHRTSIPVHSKQAEKISRRPSTEDTHEVDSKAALIPDWLQDRPSNREMG
LTSPFKPAMDTNYYYSAVERNNLMRLSQSIPFTPVPPRGEPVTVYRLEES
SPNILNNSMSSWSQLGLCAKIEFLSKEEMGGGLRRAVKVQCTWSEHDILK
SGHLYIIKSFLPEVVNTWSSIYKEDTVLHLCLREIQQQRAAQKLTFAFNQ
MKPKSIPYSPGELLVLDLQGVGENLTDPSVIKAEEKRSCDMVFGPANLGE
DAIKNFRAKHHCNSCCRKLKLPDLKRNDYTPDKIIFPQDEPSDLNLQPGN
STKESESTNSVRLML

A search of sequence databases reveals that the NOV4b amino acid sequence has 776 of 892 amino acid residues (86%) identical to, and 819 of 892 amino acid residues (91%) similar to, the 1863 amino acid residue ptnr:SPTREMBL-ACC:Q9JLQ1 protein from Mus musculus (Mouse) (TRANSIENT RECEPTOR POTENTIAL-RELATED PROTEIN). Public amino acid databases include the GenBank databases, SwissProt, PDB and PIR.

NOV4b is expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, Public EST sources, Literature sources, and/or RACE sources.

The disclosed NOV4a polypeptide has homology to the amino acid sequences shown in the BLASTP data listed in Table 4E.

TABLE 4E
BLAST results for NOV4

| Gene Index/ | | Length | Identity | Positives | |
|---|---|---|---|---|---|
| Identifier | Protein/Organism | (aa) | (%) | (%) | Expect |
| gi\|13959785\|gb\|AAK44211.1\| | LTRPC7 [Homo | 1865 | 1847/1866 | 1853/1866 | 0.0 |
| (AY032950) | sapiens] | | (98%) | (98%) | |
| gi\|13562153\|gb\|AAK19738.2\| | channel-kinase 1 | 1864 | 1845/1866 | 1851/1866 | 0.0 |
| AF346629_1 | [Homo sapiens] | | (98%) | (98%) | |
| (AF346629) | | | | | |
| gi\|14009344\|gb\|AAK50377.1\| | LTRPC7 [Mus | 1863 | 1748/1866 | 1803/1866 | 0.0 |
| (AY032951) | musculus] | | (93%) | (95%) | |
| gi\|10946830\|ref\|NP_ | transient | 1863 | 1747/1866 | 1803/1866 | 0.0 |
| 067425.1\| | receptor | | (93%) | (96%) | |
| (NM_021450) | potential M7; | | | | |
| | transient | | | | |
| | receptor | | | | |
| | potential-related | | | | |
| | protein, ChaK | | | | |
| | [Mus musculus] | | | | |
| gi\|14211383\|gb\|AAK57433.1\| | transient | 1862 | 1747/1866 | 1802/1866 | 0.0 |
| AF376052_1 | receptor | | (93%) | (95%) | |
| (AF376052) | potential | | | | |
| | phospholipase C | | | | |
| | interacting | | | | |
| | kinase [Mus | | | | |
| | musculus] | | | | |

Table 4F-G lists the domain descriptions from DOMAIN analysis results against NOV4. This indicates that the NOV4 sequence has properties similar to those of other proteins known to contain this domain.

TABLE 4F
Domain Analysis of NOV4

gnl|Pfam|pfam02816, MHCK_EF2_kinase,
MHCK/EF2 kinase domain family. This family is a novel
family of eukaryotic
protein kinase catalytic domains, which have no detectable similarity
to conventional kinases. The family contains myosin heavy chain
kinases and Elongation Factor-2 kinase and a bifunctional ion channel.
CD-Length = 206 residues, 94.7% aligned
Score = 79.7 bits (195), Expect = 1e−15

TABLE 4G
Domain Analysis of NOV4

gnl|Pfam|pfam00520, ion_trans, Ion transport protein. This family
contains Sodium, Potassium, Calcium ion channels. This family is 6
transmembrane helices in which the last two helices flank a loop which
determines ion selectivity. In some sub-families (e.g. Na channels)
the domain is repeated four times, whereas in others (e.g. K channels)
the protein forms as a tetramer in the membrane.
CD-Length = 191 residues, 99.0% aligned
Score = 62.8 bits (151), Expect = 2e−10

Capacitative calcium entry (CCE) describes CA2+ influx into cells that replenishes CA2+ stores emptied through the action of IP3 and other agents. It is an essential component of cellular responses to many hormones and growth factors. The molecular basis of this form of Ca2+ entry is complex and may involve more than one type of channel. Studies on visual signal transduction in Drosophila led to the hypothesis that a protein encoded in transient receptor potential (Trp) and related proteins may be a component of CCE channels. Zhu et al.) reported the existence of six trp-related genes in the mouse genome. Expression in L cells of small portions of these genes in antisense orientation Suppressed CCE. Expression in COS cells of two full-length cDNAs encoding human trp homologs, Htrp1 and Htrp3, increased CCE. This identifies mammalian gene products that participate in CCE.

Human TRPC genes encode proteins with sequence similarity to the Drosophila ‘transient receptor potential’ (trp) gene product. TRPC proteins are thought to be subunits of capacitative calcium entry (CCE) channels, which mediate calcium influx into cells to replenish internal stores of calcium. Using exon trapping on a contig from 21q22.3, Kudoh et al. (1997) isolated an exon whose deduced amino acid sequence shows similarity to the sequences of human TRPC and Drosophila trp proteins. Nagamine et al. (1998) isolated human fetal brain and caudate nucleus cDNAs corresponding to the exon and its parent gene. The deduced 1,503-amino acid protein, which is named TRPC7, is 22.9% identical to human TRPC1(602343), 21.2% identical to human TRPC3 (602345), and 22.6% identical to Drosophila trp. TRPC7 contains 7 predicted membrane-spanning domains. The TRPC7 gene has 32 exons spanning approximately 90 kb. Northern blot analysis of human tissues detected a 6.5-kb TRPC7 transcript predominantly in fetal and adult brains, where it was expressed in several regions. In caudate nucleus and putamen, a putative 5.5-kb alternatively spliced TRPC7 product also was detected.

The disclosed NOV4 nucleic acid of the invention encoding a TRANSIENT RECEPTOR POTENTIAL-RELATED PROTEIN-like protein includes the nucleic acid whose sequence is provided in Table 4A or 4C or a fragment thereof. The invention also includes a mutant or variant nucleic acid any of whose bases may be changed from the corresponding base shown in Table 4A or 4C while still encoding a protein that maintains its TRANSIENT RECEPTOR POTENTIAL-RELATED PROTEIN-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10 percent of the bases may be so changed.

The disclosed NOV4 protein of the invention includes the TRANSIENT RECEPTOR POTENTIAL-RELATED PROTEIN-like protein whose sequence is provided in Table 4B or 4D. The invention also includes a mutant or variant protein any of whose residues may be changed from the corresponding residue shown Table 4B or 4D while still encoding a protein that maintains its TRANSIENT RECEPTOR POTENTIAL-RELATED PROTEIN-like activities and physiological functions, or a functional fragment thereof. In the mutant or variant protein, up to about 7 percent of the residues may be so changed.

The invention further encompasses antibodies and antibody fragments, such as F_{ab} or (F_{ab})₂, that bind immunospecifically to any of the proteins of the invention.

The above defined information for this invention suggests that this TRANSIENT RECEPTOR POTENTIAL-RELATED PROTEIN-like protein (NOV4) may function as a member of a “TRANSIENT RECEPTOR POTENTIAL-RELATED PROTEIN family”. Therefore, the NOV4 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV4 nucleic acids and proteins of the invention are useful in potential therapeutic applications implicated in diseases including but not limited to various pathologies and disorders as indicated below. For example, a cDNA encoding the TRANSIENT RECEPTOR POTENTIAL-RELATED PROTEIN-like protein (NOV4) may be useful in gene therapy, and the TRANSIENT RECEPTOR POTENTIAL-RELATED PROTEIN-like protein (NOV4) may be useful when administered to a subject in need thereof. By way of nonlimiting example, the compositions of the present invention will have efficacy for treatment of patients suffering from adrenoleukodystrophy, congenital adrenal hyperplasia, hemophilia, hypercoagulation, idiopathic thrombocytopenic purpura, autoimmune disease, allergies, immunodeficiencies, transplantation, graft versus host disease, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, arthritis, tendonitis, diabetes, renal artery stenosis, interstitial nephritis, glomerulonephritis, polycystic kidney disease, systemic lupus erythematosus, renal tubular acidosis, IgA nephropathy, cirrhosis, lymphedema, ulcers, tonsillitis, or other pathologies or conditions. The NOV4 nucleic acid encoding the TRANSIENT RECEPTOR POTENTIAL-RELATED PROTEIN-like protein of the invention, or fragments thereof, may further be useful in diagnostic applications, wherein the presence or amount of the nucleic acid or the protein are to be assessed.

NOV4 nucleic acids and polypeptides are further useful in the generation of antibodies that bind immuno-specifically to the novel NOV4 substances for use in therapeutic or diagnostic methods. These antibodies may be generated according to methods known in the art, using prediction from hydrophobicity charts, as described in the “Anti-NOVX Antibodies” section below. The disclosed NOV4 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV5

A disclosed NOV5 nucleic acid of 1869 nucleotides (also referred to as CG57609-01) encoding a Epsin-3-like protein is shown in Table 5A. Putative untranslated regions upstream and/or downstream from the coding region, if any, are underlined, and the start and stop codons are in bold letters.

TABLE 5A
NOV5 nucleotide sequence.

| | |
|---|---|
| GCGGGGGCGAGGGCCACCCACCTCCAAGTCTCCAGCCATGACGACCTCCGCACTCCGGCG | (SEQ ID NO:11) |
| CCAGGTGAAGAACATCGTGCACAACTACTCCGAGGCAGAAATCAAGGTGCGCGAGGCCAC | |
| CAGCAATGACCCCTGGGGCCCCCCTAGTTCGCTCATGTCCGAGATCGCTGACCTGACCTT | |
| CAACACAGTGGCCTTCACCGAAGTCATGGGCATGCTGTGGCGGCGGCTCAATGACAGCGG | |
| CAAGAACTGGCGGCACGTGTACAAGGCTCTAACATTGCTGGACTACCTGCTCAAGACGGG | |
| CTCCGAGCGGGTGGCCCACCAGTGCCGCGAGAACCTCTACACCATCCAGACACTCAAGGA | |
| CTTCCAGTACATCGACCGCGACGGCAAGGACCAGGGCGTCAACGTGCGCGAGAAGGTCAA | |
| GCAGGTGATGGCCCTGCTCAAGGATGAGGAGCGGCTGCGGCAGGAGCGAACCCACGCCCT | |
| CAAGACCAAGGAGCGCATGGCACTGGAGGGCATCGGCCCGCTGGTGCTGGGCTTCAGCCG | |
| CCGCTACGGCGAGGACTACAGCCGCTCCCGGGGCTCCCCGTCCTCCTACAACTCCTCCTC | |
| TTCGTCACCCCGCTATACCTCCGACCTGGAGCAGGCCCGGCCTCAGACGTCAGGGGAAGA | |
| GGAACTGCAGCTGCAGCTGGCCCTCGCCATCAGCCGTGAGGAGGCAGAGAAGGAGGTGAG | |
| GTCCTGGCAGGGTGATGGCTCCCCCATGGCCAATGGTGCAGGGGCCGTGGTCCACCATCA | |
| GCGGGACAGAGAGCCTGAGAGAGAAGAGAGAAAGGAGGAGGAGAAGCTAAAAACCAGCCA | |
| GTCCTCCATCCTGGACTTGGCTGACATCTTCGTACCTGCCCTGGCCCCGCCCTCCACACA | |
| CTGCTCTGCTGACCCATGGGACATCCCAGGTTTTAGGCCGAACACACAGGCCAGTGGATC | |
| CTCCTGGGGGCCTTCTGCAGACCCCTGGTCTCCGATCCCCTCAGGAACCGTCCTGTCCCG | |
| AAGCCAGCCCTGGGATCTGACTCCCATGCTCTCCTCCTCTGAGCCCTGGGGCAGGACCCC | |
| AGTGCTGCCTGCTGGGCCCCCCACCACAGACCCCTGGGCCCTGAACTCTCCCCACCACAA | |
| ACTCCCCAGCACTGGGGCTGACCCTTGGGGAGCCTCCCTGGAGACCTCCGACACACCTGG | |
| TGGTGCCTCGACCTTTGACCCATTTGCCAAACCTCCAGAATCCACAGAGACCAAGGAGGG | |
| GCTGGAGCAGGCCCTGCCCTCTGGGAAGCCCAGCAGCAGCGGGGAGCTGGACCTGTTTGG | |
| AGACCCCAGCCCCAGTTCCAAGCAAAATGGCACGAAGGAGCCAGATGCCCTGGACCTGGG | |
| CATACTAGGGGAAGCACTAACCCAGCCAAGCAAAGAGGCCCGAGCTTGCCGGACTCCCGA | |
| GTCCTTCCTGGGTCCCTCAGCTTCCTCCTTGGTCAACCTTGACTCGTTGGTCAAGGCACC | |
| CCAGGTTGCAAAGACCCGGAACCCCTTCCTGACAGGTGGTCTCAGCGCTCCGTCCCCCAC | |
| CAACCCGTTCGGCGCGGGCGAGCCGGGCAGGCCGACGCTAAACCAGATGCGCACCGGCTC | |
| GCCGGCGCTGGGCCTGGCAGGCGGGCCTGTGGGGGCGCCCCTGGGCTCCATGACCTACAG | |
| CGCCTCTCTGCCCCTCCCGCTCAGCAGCGTGCCAGCTGGCTTGACCCTCCCCGCCTCGGT | |
| TAGCGTCTTCCCGCAGGCCGGAGCCTTCGCACCGCAGCCGCTGCTGCCCACGCCGAGCTC | |
| AGCCGGGCCGCGGCCCCCGCCCCCGCAGACCGGCACCAACCCCTTCCTCTGAGCCCCGCC | |
| CCGTCCCAT | |

In a search of public sequence databases, the NOV5 nucleic acid sequence, located on chromsome 17 has 1210 of 1234 bases (98%) identical to a gb:GENBANK-ID:AK000785|acc:AK000785.1 mRNA from Homo sapiens (Homo sapiens cDNA FLJ20778 fis, clone COL05704). Public nucleotide databases include all GenBank databases and the GeneSeq patent database.

The disclosed NOV5 polypeptide (SEQ ID NO:12) encoded by SEQ ID NO:11 has 604 amino acid residues and is presented in Table 5B using the one-letter amino acid code. Signal P, Psort and/or Hydropathy results predict that NOV5 has no signal peptide and is likely to be localized in the cytoplasm with a certainty of 0.4500.

TABLE 5B
Encoded NOV5 protein sequence.

| | |
|---|---|
| MTTSALRRQVKNIVHNYSEAEIKVREATSNDPWGPPSSLMSEIADLTFNTVAFTEVMGML | (SEQ ID NO:12) |
| WRRLNDSGKNWRHVYKALTLLDYLLKTGSERVAHQCRENLYTIQTLKDFQYIDRDGKDQG | |
| VNVREKVKQVMALLKDEERLRQERTHALKTKERMALEGIGPLVLGFSRRYGEDYSRSRGS | |
| PSSYNSSSSSPRYTSDLEQARPQTSGEEELQLQLALAMSREEAEKEVRSWQGDGSPMANG | |
| AGAVVHHQRDREPEREERKEEEKLKTSQSSILDLADIFVPALAPPSTHCSADPWDIPGFR | |
| PNTEASGSSWGPSADPWSPIPSGTVLSRSQPWDLTPMLSSSEPWGRTPVLPAGPPTTDPW | |
| ALNSPHHKLPSTGADPWGASLETSDTPGGASTFDPFAKPPESTETKEGLEQALPSGKPSS | |
| SGELDLFGDPSPSSKQNGTKEPDALDLGILGEALTQPSKEARACRTPESFLGPSASSLVN | |
| LDSLVKAPQVAKTRNPFLTGGLSAPSPTNPFGAGEPGRPTLNQMRTGSPALGLAGGPVGA | |
| PLGSMTYSASLPLPLSSVPAGLTLPASVSVFPQAGAFAPQPLLPTPSSAGPRPPPPQTGT | |
| NPFL | |

A search of sequence databases reveals that the NOV5 amino acid sequence has 398 of 441 amino acid residues (90%) identical to, and 406 of 441 amino acid residues (92%) similar to, the 632 amino acid residue ptnr:TREMBLNEW-ACC:AAG45223 protein from Homo sapiens (Human) (EPSIN 3). Public amino acid databases include the GenBank databases, SwissProt, PDB and PIR.

NOV5 is expressed in at least skin. This information was derived by determining the tissue sources of the sequences that were included in the invention including but not limited to SeqCalling sources, Public EST sources, Literature sources, and/or RACE sources.

The disclosed NOV5 polypeptide has homology to the amino acid sequences shown in the BLASTS data listed in Table 5C.

TABLE 5C
BLAST results for NOV5

| Gene Index/ | | Length | Identity | Positives | |
|---|---|---|---|---|---|
| Identifier | Protein/Organism | (aa) | (%) | (%) | Expect |
| ref\|XP_037971.1\| | epsin 3 [Homo | 632 | 598/633 | 598/633 | 0.0 |
| (XM_037971) | sapiens] | | (94%) | (94%) | |
| ref\|NP_060427.1\| | epsin 3 [Homo | 632 | 596/633 | 597/633 | 0.0 |
| (NM_017957 | sapiens] | | (94%) | (94%) | |
| gb\|AAH16454.1\|AAH16454 | (protein for | 636 | 518/646 | 535/646 | 0.0 |
| (BC016454) | MGC: 25634) [Mus | | (80%) | (82%) | |
| | musculus] | | | | |
| dbj\|BAB26309.1\| | homolog to EPSIN | 569 | 452/579 | 468/579 | 0.0 |
| (AK009469) | 3˜putative [Mus | | (78%) | (80%) | |
| | musculus] | | | | |
| gb\|AAH01038.1\|AAH01038 | Similar to epsin | 208 | 186/205 | 188/205 | e−101 |
| (BC001038) | 3 [Homo sapiens] | | (90%) | (90%) | |

Tables 5D-E list the domain descriptions from DOMAIN analysis results against NOV5. This indicates that the NOV sequence has properties similar to those of other proteins known to contain this domain.

TABLE 5E
Domain Analysis of NOV5

gnl|Pfam|pfam01417, ENTH, ENTH domain. The ENTH
(Epsin N-terminal homology) domain is found in proteins involved
in endocytosis and cytoskeletal machinery.
CD-Length = 123 residues, 100.0% aligned
Score = 174 bits (442), Expect = 1e−44

TABLE 5F
Domain Analysis of NOV5

gnl|Smart|smart00288, VHS, Domain present in VPS-27,
Hrs and STAM; Unpublished observations
CD-Length = 133 residues, 88.7% aligned
Score = 38.5 bits (88), Expect = 0.001

The mammalian protein epsin is required for endocytosis. There are two homologous yeast proteins, Ent1p and Ent2p, which are similar to mammalian epsin. An essential function for the highly conserved N-terminal epsin N-terminal homology (ENTH) domain was revealed using deletions and randomly generated temperature-sensitive ent1 alleles. Changes in conserved ENTH domain residues in ent1(ts) cells revealed defects in endocytosis and actin cytoskeleton structure. The Ent1 protein was localized to peripheral and internal punctate structures, and biochemical fractionation studies found the protein associated with a large, Triton X-100-insoluble pellet. Finally, an Ent1p clathrin-binding domain was mapped to the final eight amino acids (RGYTLIDL*) in the Ent1 protein sequence. Based on these and other data, yeast epsin-like proteins are essential components of an endocytic complex that may act at multiple stages in the endocytic pathway.

An approximately 140 amino acid domain is shared by a variety of proteins in budding and fission yeast, nematode, rat, mouse, frog, oat, and man. Typically, this domain is located within 20 residues of the N-terminus of the various proteins. The percent identity among the domains in the 12 proteins ranges from 42 to 93%, with 16 absolutely conserved residues. Even though these proteins share little beyond their segment of homology, data are emerging that several of the proteins are involved in endocytosis and or regulation of cytoskeletal organization. This protein segment is the ENTH domain, for Epsin N-terminal homology domain.

The disclosed NOV5 nucleic acid of the invention encoding a Epsin-3-like protein includes the nucleic acid whose sequence is provided in Table 5A or a fragment thereof. The invention also includes a mutant or variant nucleic acid any of whose bases may be changed from the corresponding base shown in Table 5A while still encoding a protein that maintains its Epsin-3-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2 percent of the bases may be so changed.

The disclosed NOV5 protein of the invention includes the Epsin-3-like protein whose sequence is provided in Table 5B. The invention also includes a mutant or variant protein any of whose residues may be changed from the corresponding residue shown in Table 5B while still encoding a protein that maintains its Epsin-3-like activities and physiological functions, or a functional fragment thereof. In the mutant or variant protein, up to about 10 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Epsin-3-like protein (NOV5) may function as a member of a “Epsin-3 family”. Therefore, the NOV5 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV5 nucleic acids and proteins of the invention are useful in potential therapeutic applications implicated in diseases including but not limited to various pathologies and disorders as indicated below. For example, a cDNA encoding the Epsin-3-like protein (NOV5) may be useful in gene therapy, and the Epsin-3-like protein (NOV5) may be useful when administered to a subject in need thereof. By way of nonlimiting example, the compositions of the present invention will have efficacy for treatment of patients suffering from psoriasis, actinic keratosis, tuberous sclerosis, acne, hair growth/loss, allopecia, pigmentation disorders, endocrine disorders, or other pathologies or conditions. The NOV5 nucleic acid encoding the Epsin-3-like protein of the invention, or fragments thereof, may further be useful in diagnostic applications, wherein the presence or amount of the nucleic acid or the protein are to be assessed.

NOV5 nucleic acids and polypeptides are further useful in the generation of antibodies that bind immuno-specifically to the novel NOV5 substances for use in therapeutic or diagnostic methods. These antibodies may be generated according to methods known in the art, using prediction from hydrophobicity charts, as described in the “Anti-NOVX Antibodies” section below. The disclosed NOV5 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV6

A disclosed NOV6 nucleic acid of 2646 nucleotides (also referred to as CG57611-01) encoding a CD22-like protein is shown in Table 6A. Putative untranslated regions upstream and/or downstream from the coding region, if any, are underlined, and the start and stop codons are in bold letters.

TABLE 6A
NOV6 nucicotide sequence.

| | |
|---|---|
| ATGGACAACCCACAGGCTCTGCCACTCTTCCTACTCCTGGCCTCCTTGGTAGGGATCCTC | (SEQ ID NO:13) |
| ACCCTCAGAGCCTCTTCTGGACTTCACCAAACCAACTTCTCCTCTGCCTTCTCTTCAGAC | |
| TCAAAGAGCTCTTCCCAGGGGCTGGGTGTGGAAGTTCCCTCCATCAAACCTCCCAGCTGG | |
| AAAGTTCCAGATCAGTTCCTGGATTCAAAAGCCTCTGCTGGAATCTCTGATTCCAGCTGG | |
| TTTCCTGAGGCCCTGAGTTCCAACATGTCTGGGTCCTTCTGGTCAAATGTTTCTGCTGAG | |
| GGCCAAGATTTGAGCCCGGTTTCCCCCTTCTCTGAAACCCCTGGTTCTGAAGTATTTCCT | |
| GATATTTCGGATCCTCAAGTTCCTGCCAAAGACCCCAACCCTTCCTTCACTGTTAAGACC | |
| CCAGCTTCAAACATTTCTACTCAAGTCTCCCATACCAAACTGTCTGTTGAGGCCCCAGAT | |
| TCAAAATTCTCCCCGGATGATATGGATCTTAAACTCTCTGCCCAGAGCCCTGAATCCAAA | |
| TTTTCTGCAGAGACCCACTCAGCTGCAAGCTTTCCCCAGCAGGTGGGGGGCCCACTCGCT | |
| GTGCTGGTGGGGACCACCATCCGGCTCCCCCTAGTCCCAATCCCCAACCCTGGGCCCCCC | |
| ACCTCTCTGGTGGTCTGGCGCCGGGGCTCAAAGGTGCTGGCAGCTGGGGGCCTGGGGCCA | |
| GGGGCACCTCTGATCAGCCTGGACCCTGCTCACCGAGACCACCTGCGATTTGACCAGGCC | |
| CGGGGGGTTCTGGAGCTCGCCTCTGCCCAGCTGGACGATGCAGGGGTCTACACGGCTGAG | |
| GTCATCCGGGCAGGGGTCTCCCAGCAGACTCACGAGTTCACGGTGGGTGTGTATGAGCCC | |
| CTACCCCAGCTGTCGGTTCAGCCCAAGGCTCCAGAGACAGAGGAGGGGGCGGCCGAGCTC | |
| CGGCTGCGCTGCCTGGGGTGGGGGCCAGGTCGCGGGGAGCTGAGCTGGAGCCGGGACGGA | |
| CGCGCCCTGGAGGCGGCGGAATCGGAGGGAGCCGAGACGCCCCGGATGCGCTCAGAGGGC | |
| GACCAGCTGCTCATCGTGCGCCCTGTGCGCAGCGACCACGCCCGGTACACTTGCCGCGTC | |
| CGCAGCCCCTTCGGCCACAGGGAGGCTGCCGCCGACGTCAGCGTCTTCTACGGCCCGGAC | |
| CCGCCGACCATCACGGTCTCCTCGGACCGCGACGCCGCGCCTGCCCGCTTTGTCACCGCG | |
| GGCAGTAACGTGACCTTGCGCTGCGCCGCCGCCTCGCGGCCGCCCGCCGACATCACGTGG | |
| AGCCTGGCGGACCCGGCCGAGGCCGCGGTGCCCGCGGGGTCCCGCCTCCTGCTGCCCGCG | |
| GTCGGACCGGGCCACGCAGGCACCTACGCCTGCCTGGCGGCGAACCCGCGTACCGGCCGC | |
| CGCCGCCGCTCGCTGCTCAACCTTACAGTGGCGGACCTGCCCCCCGGGGCCCCACAGTGC | |
| TCAGTTGAAGGGGGTCCCGGGGACCGCAGCCTCCGCTTCCGCTGCTCGTGGCCCGGCGGG | |
| GCCCCTGCTGCCTCCCTGCAGTTCCAGGGTCTCCCCGAAGGCATCCGCGCCGGGCCAGTG | |
| TCCTCTGTGCTGCTGGCGGCCGTCCCCGCCCACCCCCGGCTCAGCGGCGTCCCCATCACC | |
| TGCCTTGCTCGCCACCTGGTGGCCACGCGTACCTGCACAGTCACGCCGGAGGCCCCCCGA | |
| GAGGTGCTGCTGCATCCGCTGGTGGCAGAGACACGGTTGGGGGAGGCAGAGGTGGCACTG | |
| GAGGCCTCTGGTTGTCCCCCACCCTCACGGGCATCCTGGGCCCGGGAAGGGAGGCCCCTG | |
| GCTCCAGGAGGCGGGAGTCGCCTGCGGCTCAGTCAAGATGGCCGGAAACTCCACATCGGC | |
| AACTTCAGCCTGGATTGGGACCTGGGAAATTACTCCGTGCTGTGCAGTGGGGCGCTGGGT | |
| GCTGGCGGTGACCAGATCACCCTCATTGGACCCTCCATATCCTCGTGGAGGCTTCAGAGA | |
| GCCAGAGATGCAGCCGTGCTGACTTGGGATGTGGAGCGCGGGGCCCTGATCAGCAGTTTT | |
| GAGATCCAGGCATGGCCAGATGGGCCTGCTCTGGGCAGGACTTCCACCTACAGGGACTGG | |
| GTCTCCCTGCTCATCCTGGGGCCTCAGGAGCGGTCAGCCGTGGTGCCCCTTCCACCTCGG | |
| AACCCAGGGACCTCCACCTTTCGGATCCTCCCCATCCTGGGGGGCCAGCCAGGGACTCCA | |
| TCACAAAGCCGGGTCTACCGGGCCGGCCCCACGTTGAGCCATGGGGCCATCGCTGGCATC | |
| GTCCTGGGCTCCCTGCTGGGCCTGGCGCTGCTAGCCGTACTTCTCCTCCTTTGCATCTGC | |
| TGCCTGTGCCGCTTTCGTGGAAAGACTCCTGAGAAAAAGAAGCATCCTTCTACCTTGGTC | |
| CCCGTGGTCACCCCCTCAGAAAAGAAGATGCATAGTGTGACCCCAGTGGAGATTTCATGG | |
| CCTCTGGACCTCAAAGTCCCTCTGGAGGACCACAGCTCAACTAGGGCCTACCAAAAGAAG | |
| AGTCTTCCTGTATTTGTGCAAGTAAGGAGATGTGACTTCTTGGCTGGGAAACTCTTGCTG | |
| ATCTAA | |

In a search of public sequence databases, the NOV6 nucleic acid sequence, located on chromsome 19 has 941 of 1572 bases (59%) identical to a gb:GENBANK-ID:AF246990|acc:AF246990.1 mRNA from Chlamydomonas reinhardtii (Chlamydomonas reinhardtii flagellar autotomy protein Fa1p (FA1) mRNA, complete cds). Public nucleotide databases include all GenBank databases and the GeneSeq patent database.

The disclosed NOV6 polypeptide (SEQ ID NO:14) encoded by SEQ ID NO:13 has 881 amino acid residues and is presented in Table 6B using the one-letter amino acid code. Signal P, 1-sort and/or Hydropathy results predict that NOV6 has a signal peptide and is likely to be localized at the plasma membrane with a certainty of 0.4600. The signal peptide is predicted by SignalP to be cleaved at amino acid 27-28.

TABLE 6B
Encoded NOV6 protein sequence.

| | |
|---|---|
| MDNPQALPLFLLLASLVGILTLRASSGLQQTNFSSAFSSDSKSSSQGLGVEVPSIKPPSW | (SEQ ID NO:14) |
| KVPDQFLDSKASAGISDSSWFPEALSSNMSGSFWSNVSAEGQDLSPVSPFSETPGSEVFP | |
| DISDPQVPAKDPKPSFTVKTPASNISTQVSHTKLSVEAPDSKFSPDDMDLKLSAQSPESK | |
| FSAETHSAASFPQQVGGPLAVLVGTTIRLPLVPIPNPGPPTSLVVWRRGSKVLAAGGLGP | |
| GAPLISLDPAHRDHLRFDQARGVLELASAQLDDAGVYTAEVIRAGVSQQTHEFTVGVYEP | |
| LPQLSVQPKAPETEEGAAELRLRCLGWGPGRGELSWSRDGRALEAAESEGAETPRMRSEG | |
| DQLLIVRPVRSDHARYTCRVRSPFGHREAAADVSVFYGPDPPTITVSSDRDAAPARFVTA | |
| GSNVTLRCAAASRPPADITWSLADPAEAAVPAGSRLLLPAVGPGHAGTYACLAANPRTGR | |
| RRRSLLNLTVADLPPGAPQCSVEGGPGDRSLRFRCSWPGGAPAASLQFQGLPEGIRAGPV | |
| SSVLLAAVPAHPRLSGVPITCLARHLVATRTCTVTPEAPREVLLHPLVAETRLGEAEVAL | |
| EASGCPPPSRASWAREGRPLAPGGGSRLRLSQDGRKLHIGNFSLDWDLGNYSVLCSGALG | |
| AGGDQITLIGPSISSWRLQRARDAAVLTWDVERGALISSFEIQAWPDGPALGRTSTYRDW | |
| VSLLILGPQERSAVVPLPPRNPGTWTFRILPILGGQPGTPSQSRVYRAGPTLSHGAIAGI | |
| VLGSLLGLALLAVLLLLCICCLCRFRGKTPEKKKHPSTLVPVVTPSEKKMHSVTPVEISW | |
| PLDLKVPLEDHSSTRAYQKKSLPVFVQVRRCDFLAGKLLLI | |

A search of sequence databases reveals that the NOV6 amino acid sequence has 76 of 254 amino acid residues (29%) identical to, and 117 of 254 amino acid residues (46%) similar to, the 521 amino acid residue ptnr:SPTREMBL-ACC:Q61352 protein from Mus musculus (Mouse) (BILIARY GLYCOPROTEIN 1 PRECURSOR). Public amino acid databases include the GenBank databases, SwissProt, PDB and PIR.

NOV6 is expressed in at least lung, ovary, squamous cell carcinoma, and fibrotheoma. This information was derived by determining the tissue sources of the sequences that were included in the invention including but not limited to SeqCalling sources, Public EST sources, Literature sources, and/or RACE sources.

The disclosed NOV6 polypeptide has homology to the amino acid sequences shown in the BLASTP data listed in Table 6C.

TABLE 6C
BLAST results for NOV6

| Gene Index/ | | Length | Identity | Positives | |
|---|---|---|---|---|---|
| Identifier | Protein/Organism | (aa) | (%) | (%) | Expect |
| gi\|312584\|emb\|CAA47697.1\| | biliary | 458 | 68/237 | 109/237 | 1e−16 |
| (X67280) | glycoprotein [Mus | | (28%) | (45%) | |
| | musculus] | | | | |
| gi\|14029256\|gb\|AAK52602.1\| | CEA-related cell | 520 | 68/237 | 109/237 | 3e−16 |
| (AF287912) | adhesion molecule | | (28%) | (45%) | |
| | 2 [Mus musculus] | | | | |
| gi\|423398\|pir\|\|S34338 | biliary | 521 | 68/237 | 109/237 | 4e−16 |
| | glycoprotein F - | | (28%) | (45%) | |
| | mouse | | | | |
| gi\|483309\|pir\|\|JC1509 | biliary | 458 | 68/237 | 108/237 | 5e−16 |
| | glycoprotein E - | | (28%) | (44%) | |
| | mouse | | | | |
| gi\|109630\|pir\|\|S11626 | carcinoembryonic | 379 | 66/237 | 106/237 | 1e−15 |
| | antigen - mouse | | (27%) | (43%) | |
| | (fragment) | | | | |

Table 6D lists the domain descriptions from DOMAIN analysis results against NOV6. This indicates that the NOV6 sequence has properties similar to those of other proteins known to contain this domain.

TABLE 61D
Domain Analysis of NOV6

gnl|Smart|smart00408, IGc2, Immunoglobulin C-2 Type
CD-Length = 63 residues, 93.7% aligned
Score = 55.5 bits (132), Expect = 1e−08

The disclosed NOV6 novel gene described here contains three immunoglobulin domains and has homology to mouse CD22, a B lymphocyte-restricted adhesion molecule, mouse colon biliary glycoprotein, and carcinoembryonic antigen. The immunoglobulin domain is found as a tandem repeat in Streptococcal cell surface proteins, such as the IgG binding proteins G and MIG. These proteins are type I membrane proteins that bind to the constant Fc region of IgG with high affinity. The N-terminus of MIG mediates binding to plasma proteinase inhibitor alpha 2-macroglobulin after complex formation with proteases.

The human B lymphocyte-specific Ag, CD22, is a cell adhesion molecule expressed on the surface during a narrow window of B cell development, coincident with surface IgD. A ligand for CD22 has recently been identified on human T cells as the low molecular mass isoform of the leukocyte common Ag, CD45RO. CD22 has been reported to function in the regulation of both T and B cell activation in vitro.

Carcinoembryonic antigen (CEA) is a widely used tumor marker, especially in the surveillance of colonic cancer patients. Although CEA is also present in some normal tissues, it is apparently expressed at higher levels in tumorous tissues than in corresponding normal tissues. Carcinoembryonic antigen (CEA) expression is perhaps the most prevalent of phenotypic changes observed in human cancer cells. Twenty-seven CEA cDNA clones were isolated from a human colon adenocarcinoma cell line. Most of these clones are full length and consist of a number (usually three) of surprisingly similar long (534 base pairs) repeats between a 5′ end of 520 base pairs and a 3′ end with three different termination points. The predicted translation product of these clones consists of a processed signal sequence of 34 amino acids, an amino-terminal sequence of 107 amino acids, which includes the known terminal amino acid sequence of CEA, three repeated domains of 178 amino acids each, and a membrane-anchoring domain of 27 amino acids, giving a total of 702 amino acids and a molecular weight of 72,813 for the mature protein. The repeated domains have conserved features, including the first 67 amino acids at their N termini and the presence of four cysteine residues. Comparisons with the amino acid sequences of other proteins reveals homology of the repeats with various members of the immunoglobulin supergene family, particularly the human T-cell receptor gamma chain. CEA cDNA clones in the SP-65 vector were shown to produce transcripts in vitro which could be translated in vitro to yield a protein of molecular weight 73,000 which in turn could be precipitated with CEA-specific antibodies (See Schrewe H et al., Mol Cell Biol 1990 June;10(6):2738-48.).

The biliary glycoprotein (BGP)-encoding gene is a member of the human carcinoembryonic antigen (CEA) gene family. McCuaig et al. cloned several mouse Bgp cDNAs from an outbred CDR-1 mouse colon cDNA library, as well as by reverse transcription-PCR amplification of colon RNA. The distinguishing features of the deduced Bgp protein isoforms are found in the two divergent N-terminal domains, the highly conserved internal C2-set immunoglobulin domains, and an intracytoplasmic domain of either 10 or 73 amino acids (aa). The cDNA structures suggest that these mRNAs are produced through alternative splicing of a Bgp gene and the usage of multiple transcriptional terminators. The Bgp deduced aa sequences are highly homologous to several well characterized rat hepatocyte proteins such as the cell CAM105/ecto-ATPase/pp120/HA4 proteins. Oligodeoxyribonucleotide probes representing the various cDNA isoform domains revealed predominant transcripts of 1.8, 3.1 and 4.0 kb on Northern analyses of mouse colon RNA; some of these bands are actually composed of several co-migrating transcripts. The transcripts encoding the long intracytoplasmic-tailed Bgp proteins are expressed at one-tenth the relative abundance of the shorter-tailed species. The expression of the many Bgp isoforms at the surface of epithelial cells, such as colon, suggests that these proteins play a determinant role, through self- or heterologous contact, in renewal and/or differentiation of their epithelia (See McCuaig et al., Gene May 30, 1993;127(2):173-83).

The disclosed NOV6 nucleic acid of the invention encoding a CD22-like protein includes the nucleic acid whose sequence is provided in Table 6A or a fragment thereof. The invention also includes a mutant or variant nucleic acid any of whose bases may be changed from the corresponding base shown in Table 6A while still encoding a protein that maintains its CD22-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 41 percent of the bases may be so changed.

The disclosed NOV6 protein of the invention includes the CD22-like protein whose sequence is provided in Table 6B. The invention also includes a mutant or variant protein any of whose residues may be changed from the corresponding residue shown in Table 6B while still encoding a protein that maintains its CD22-like activities and physiological functions, or a functional fragment thereof. In the mutant or variant protein, tip to about 71 percent of the residues may be so changed.

The invention further encompasses antibodies and antibody fragments, such as F_{ab} or (F_{ab})₂, that bind immunospecifically to any of the proteins of the invention.

The above defined information for this invention suggests that this CD22-like protein (NOV6) may function as a member of a “CD22 family”. Therefore, the NOV6 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV6 nucleic acids and proteins of the invention are useful in potential therapeutic applications implicated in diseases including but not limited to various pathologies and disorders as indicated below. For example, a cDNA encoding the CD22-like protein (NOV6) may be useful in gene therapy, and the CD22-like protein (NOV6) may be useful when administered to a subject in need thereof. By way of nonlimiting example, the compositions of the present invention will have efficacy for treatment of patients suffering from endometriosis, fertility, systemic lupus erythematosus, autoimmune disease, asthma, emphysema, scleroderma, allergy, ARDS, or other pathologies or conditions. The NOV6 nucleic acid encoding the CD22-like protein of the invention, or fragments thereof, may further be useful in diagnostic applications, wherein the presence or amount of the nucleic acid or the protein are to be assessed.

NOV6 nucleic acids and polypeptides are further useful in the generation of antibodies that bind immuno-specifically to the novel NOV6 substances for use in therapeutic or diagnostic methods. These antibodies may be generated according to methods known in the art, using prediction from hydrophobicity charts, as described in the “Anti-NOVX Antibodies” section below. The disclosed NOV6 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV7

A disclosed NOV7 nucleic acid of 8589 nucleotides (also referred to as CG57595-01) encoding a MEGF8-like protein is shown in Table 7A. Putative untranslated regions upstream and/or downstream from the coding region, if any, are underlined, and the start and stop codons are in bold letters.

TABLE 7A
NOV7 nucleotide sequence.

(SEQ ID NO:15)

TGCAGGAGGCGGCGATGGCCCTGGGCAAGGTTCTGGCCATGGCACTGGTTTTGGCCTTGGCCGTGCTGGG
GTCGCTGTCCCCTGGGGCCCGGGCGGGGGACTGCAAGGGGCAGCGGCAGGTGCTGCGGGAGGCGCCAGGC
TTCGTGACGGATGGTGCGGGCAACTACAGCGTCAATGGCAACTGCGAGTGGCTCATCGAGGCCCCAAGCC
CCCAGCACCGGATCCTGCTGGACTTCCTTTTCCTGGACACAGAGTGCACGTATGACTACCTGTTCGTGTA
TGACGGTGACTCCCCGCGAGGGCCGCTGCTTGCCAGTCTAAGTGGGAGCACCCGACCTCCGCCCATCGAA
GCTTCCTCAGGCAAGATGCTGCTGCACCTCTTCAGTGATGCCAACTACAACCTGCTGGGCTTTAACGCCT
CATTCCGCTTCTCCCTGTGCCCGGGTGGCTGCCAGAGCCACGGGCAGTGCCAGCCACCGGGTGTGTGTGC
CTGCGAGCCGGGCTGGGGGGGTCCTGACTGTGGCCTGCAGGAGTGCTCAGCCTACTGTGGCAGCCACGGC
ACCTGCGCCTCGCCCCTGGGACCATGCCGCTGTGAGCCTGGCTTCTTGGGACGTGCCTGTGACCTGCACC
TGTGGGAGAACCAGGGGGCTGGGTGGTGGCACAACGTGAGTGCCAGGGACCCTGCCTTCTCTGCCCGTAT
TGGGGCAGCTGGCGCCTTCCTGTCCCCACCAGGGCTGCTGGCAGTTTTCGGAGGCCAGGACCTCAACAAT
GCCCTGGGTGACCTCGTCCTATACAACTTCTCCGCCAACACCTGGGAGTCTTGGGACCTGAGTCCTGCCC
CGGCTGCCCGTCACTCCCATGTGGCCGTGGCCTGGGCCGCCTCCCTGGTACTGATGGGTGGTGAGCTGGC
TGACGGCTCGCTCACCAACGACGTGTGGGCCTTCAGTCCACTGGGCAGGGGCCACTGGGAGCTCCTGCCA
CCACCTGCCTCCAGCTCCTCGGGGCCCCCAGGCCTGGCAGGTCACGCGGCTGCCCTGGTGGATGATGTCT
GGCTATATGTGTCTGGAGGCCGCACCCCGCACGACCTCTTCTCCTCTCGCCTCTTCCGTTTCCCCCTTGA
CAGCACCAGCGGGGGCTATTGGGAGCAGGTGATTCCGGCAGGCGGACGGCCCCCTGCTGCCACTGGCCAC
TCCATGGTGTTCCATGCCCCCTCCCGTGCCCTGCTGGTCCATGGTGGACACCGGCCCTCCACTGCCCGGT
TCTCTGTGCGAGTGAACTCCACTGAGCTTTTCCACGTGGATCGGCATGTGTCGACGACGCTGAAGGGGCG
GGATGGGCTTCAGGGCCCAAGGGAGCGAGCCTTCCACACAGCCAGTGTTCTGGGCAATTACATGGTGGTC
TATGGGGGCAATGTGCACACCCATTACCAGGAGGAAAAGTGCTACGAAGATGGCATCTTCTTCTACCACC
TTGGCTGCCATCAATGGGTGTCAGGAGCTGAGCTTGCCCCGCCAGGAACCCCTGAGGGCCGAGCAGCGCC
TCCCAGTGGTCGGTACTCACATGTAGCTGCGGTGCTTGGTGGCAGCGTCCTGTTGGTGGCTGGGGGGTAC
AGCGGCCGGCCCCGTGGGGACTTGATGGCGTACAAGGTGCCCCCCTTTGTGTTCCAGGCACCTGCCCCTG
ACTACCACTTGGACTACTGCTCCATGTACACAGACCACAGCGTCTGCTCCCGGGACCCGGAATGCAGTTG
GTGCCAAGGAGCCTGCCAAGCTGCACCCCCTCCTGGGACCCCCTTGGGGGCTTGTCCAGCCGCCAGCTGC
CTGGGCCTGGGCCGCCTCCTGGGTGACTGCCAGGCCTGCCTGGCCTTCAGCAGCCCCACAGCCCCTCCAC
GGGGACCTGGCACCCTGGGCTGGTGCGTGCACAATGAGAGCTGCCTCCCTAGGCCTGAGCAGGCCCGCTG
CCGAGGGGAGCAGATCTCAGGCACTGTGGGCTGGTGGGGGCCTGCGCCTGTCTTCGTCACGTCCCTGGAG
GCCTGCGTCACCCAGAGCTTCCTGCCTGGCCTGCACTTGCTCACCTTTCAGCAGCCGCCCAATACCTCCC
AGCCTGACAAGGTCTCAATTGTCCGCAGCACGACCATCACCCTAACACCCAGCGCAGAGACAGATGTGTC
CCTGGTCTACCGTGGCTTCATCTACCCAATGCTGCCTGGAGGGCCAGGTGGACCAGGGGCTGAGGACGTG
GCCGTGTGGACGCGGGCCCAGCGCCTACACGTCCTGGCCCGGATGGCCCGTGGCCCTGACACGGAGAACA
TGGAGGAGGTGGGGCGCTGGGTGGCTCATCAGGAGAAGGAGACGCGGCGGCTGCAGCGCCCTGGGTCTGC
TCGCCTCTTCCCTCTGCCTGGGCGGGACCACAAGTATGCAGTAGACATCCAGGGCCAGCTCAATGGCTCG
GCAGGCCCTGGGCACAGCGAGCTAACTCTGCTGTGGGATCGGACTGGTGTGCCAGGAGGCAGCGAGATCT
CCTTCTTCTTCCTGGAGCCCTACCGCTCGTCGTCCTGCACCTCCTATTCTTCCTGCCTGGGCTCCTTGGC
ACACCAGGGCTGTGGCTGGTGCCTGACCAGTGCCACCTGCCACCTGCGCCAGGGCGGAGCCCATTGCGGG
GATGACGGCGCTGGTGGGTCCCTGCTGGTGCTGGTGCCTACCCTCTGCCCACTCTGCGAGGAGCATCGGG
ACTGCCACGCCTGCACCCAGGACCCCTTCTGTGAGTGGCATCACAGCACCAGCCGCAAAGGGGACGCGGC
ATGCAGCCGGCGGGGCCGGGGTCGGGGTGCCCTGAAGAGTCCAGAGGAGTGTCCCCCGCTCTGCAGCCAG
CGACTGACCTGTGAGGACTGCCTGGCCAACTCTAGCCAGTGCGCCTGGTGCCAGTCCACCCACACCTGCT
TCCTGTTTGCTGCCTACTTGGCCCGGTACCCACACGGGGGCTGTCGAGGCTGGGACGACAGTGTACACTC
GGAGCCACGGTGCCGGAGCTGCGATGGCTTCCTGACCTGCCATGAGTGTCTGCAGAGCCACGAGTGTGGC
TGGTGTGGCAATGAGGACAACCCCACACTGGGACGGTGCCTACAGGGGGACTTCTCAGGGCCCCTCGGTG
GGGGTAACTGCTCCCTGTGGGTGGGGGAGGGCCTGGGGCTTCCCGTGGCCCTCCCTGCCCGCTGGGCATA
CGCCCGCTGTCCTGACGTGGATGAGTGTCGCCTGGGCCTGGCCCGGTGCCACCCGCGGGCGACCTGCCTG
AACACGCCCCTCAGCTACGAGTGTCACTGCCAGCGGGGCTACCAGGGTGATGGCATCTCACACTGCAACC
GCACGGATGGCATCTCACACTGCAACCGCACGTGCTTGGAGGACTGTGGCCATGGTGTGTGCAGTGGCCC
CCCGGACTTTACCTGCGTGTGTGACCTAGGCTGGACATCAGACCTGCCCCCTCCCACACCCGCCCCGGGT
CCGCCAGCCCCCCGCTGCTCCCGGGACTGTGGCTGCAGCTTCCACAGCCACTGCCGCAAGCGGGGCCCTG
GCTTCTGCGACGAGTGCCAGGACTGGACATGGGGGGAGCACTGCGAACGATGCCGGCCCGGCAGCTTCGG
CAACGCCACAGGCTCTAGGGGCTGCCGGCCCTGCCAGTGCAACGGGCACGGGGACCCACGCCGTGGCCAC
TGCGACAACCTCAGTGGGCTCTGCTTCTGCCAGGACCACACCGAGGGTGCCCACTGCCAGCTCTGCTCCC
CAGGCTATTATGGGGATCCCCGGGCCGGTGGTTCCTGCTTTCGGGAGTGTGGGGGTCGCGCCCTCCTCAC
CAACGTGTCCTCAGTGGCACTGGGCTCACGCCGGGTCGGGGGGCTGCTGCCTCCAGGTGGCGGGGCTGCA
AGAGCCGGGCCTGGCCTGTCCTACTGTGTGTGGGTTGTCTCGGCCACTGAGGAGCTACAGCCCTGTGCTC
CCGGGACCCTCTGTCCCCCACTCACCCTCACCTTCTCCCCCGACAGCAGCACCCCCTGCACCCTGAGCTA
CGTCCTGGCGTTTGATGGATTCCCACGCTTCCTGGACACTGGTGTTGTCCAGTCGGACCGCAGCCTCATA
GCTGCCTTCTGCGGCCAGCGACGGGACAGGCCCCTCACTGTTCAGGCCCTGTCTGGGCTGCTCGTGCTGC
ACTGGGAGGCCAATGGCTCCTCATCCTGGGGCTTCAATGCTTCGGTGGGCTCTGCCCGCTGTGGGTCAGG
GGGCCCCGGGAGCTGTCCCGTCCCCCAGGAATGCGTGCCCCAGGACGGTGCTGCAGGTGCGGGGCTCTGC
CGATGTCCTCAGGGCTGGGCTGGCCCACACTGCCGCATGGCTCTGTGTCCTGAGAACTGCAATGCCCACA
CTGGGGCAGGAACTTGTAACCAGAGCCTGGGTGTGTGCATCTGTGCCGAGGGCTTCGGGGGCCCCGACTG
CGCCACCAAGCTGGATGGCGGGCAGCTGGTCTGGGAGACCCTCATGGACAGCCGCCTCTCAGCCGACACT
GCCAGCCGCTTCCTGCACCGCCTGGGCCACACCATGGTGGATGGACCCGATGCCACCTTGTGGATGTTTG
GGGGCCTGGGCCTGCCCCAGGGGCTGCTGGGAAACCTGTACAGGTACTCAGTGAGTGAGCGGCGGTGGAC
ACAGATGCTGGCGGGAGCCGAGGACGGGGGCCCAGGCCCATCGCCCCGCTCCTTCCATGCAGCCGCATAT
GTGCCCGCTGGCCGTGGTGCCATGTATCTGCTGGGGGGACTTACCGCTGGAGGCGTCACCCGTGATTTCT
GGGTCCTCAACCTCACCACCCTGCAATGGCGGCAGGAGAAGGCCCCCCAGACCGTGGAGCTGCCAGCCCT
TGCTGGTCACACCCTTACTGCCCGCCGAGGCCTGTCTCTGCTCCTGGTGGGCGGTTACTCCCCGGAAAAT
GGCTTCAACCAGCAGCTGCTGGAGTACCAGCTGGCAACCGGCACCTGGGTGTCAGGAGCCCAGAGTGGGA
CACCCCCCACAGGTCTCTATGGTCACTCTGCTCTCTACCACGAGGCCACCGACTCCCTCTACGTGTTTGG
GGGGTTCCGATTCCATGTGGAGCTGGCGGCCCCATCCCCCGAGCTCTACTCCCTGCACTGTCCTGACCGC
ACCTGGAGTCTGCTGGCCCCTTCTCAGGGGGCAAAGCGAGATCGTATGAGGAATGTGCGTGGCTCATCTC
GGGGTCTGGGCCAAGTTCCTGGGGAGCAGCCTGGGTCATGGGGGTTCCGGGAAGTCAGGAACAAGATGGC
TCTGTGGGCTGCTCTTGCTGGTACAGGAGGTTTCCTGGAGGAAATCTCACCTCACCTGAAGGAGCCCCGC
CCCCGGCTTTTCCACGCCTCAGCCCTGTTAGGGGACACCATGGTGGTTCTTGGGGGGCGCTCGGACCCTG
ACGAGTTCAGCAGCGACGTTCTGCTCTACCAGGTCAACTGCAATGCCTGGCTTCTGCCCGACCTCACCCG
CTCGGCCTCTGTGGGGCCCCCAATGGAGGAGTCTGTGGCCCATGCTGTGGCAGCAGTCGGGAGCCGCCTG
TATATCTCTGGGGGTTTCGGGGGAGTGGCCCTGGGCCGCCTGCTGGCACTGACCCTGCCCCCTGACCCCT
GCCGCCTGCTGTCCTCACCTGAAGCTTGTAACCAGTCTGGGGCCTGCACCTGGTGCCATGGGGCCTGCTT
GTCCGGGGATCAGGCCCACAGGCTGGGCTGCGGGGGCTCCCCCTGCTCCCCAATGCCTCGCTCCCCGGAG
GAATGTCGACGTCTCCGGACCTGCAGTGAGTGCCTGGCCCGCCATCCTCGGACCCTCCAACCTGGAGATG
GAGAGGCGTCCACCCCCCGCTGTAAGTGGTGTACCAACTGCCCCGAAGGTGCTTGCATTGGACGCAATGG
GTCCTGCACCTCTGAGAATGACTGTCGGATCAACCAGCGAGAGGTCTTCTGGGCAGGGAACTGCTCCGAG
GCTGCGTGCGGGGCTGCTGACTGCGAGCAGTGCACGCGGGAGGGCAAGTGCATGTGGACGCGGCAGTTCA
AGAGGACAGGGGAGACCCGCCGCATCCTCTCCGTGCAGCCCACCTATGACTGGACGTGCTTCAGCCACTC
TCTGCTGAATGTGTCCCCCATGCCGGTGGAATCATCACCCCCACTGCCCTGCCCCACCCCTTGTCACCTC
CTACCCAACTGTACCTCCTGCCTGGACTCTAAGGGAGCAGATGGGGGCTGGCAGCACTGTGTTTGGAGCA
GCAGCCTGCAGCAGTGTCTGAGCCCTTCCTACCTGCCCCTGCGATGTATGGCCGGAGGCTGTGGGCGGCT
GCTCCGGGGACCTGAGAGCTGCTCCCTGGGCTGTGCTCAGGCAACTCAGTGCGCCTTGTGCCTGCGGCGC
CCCCATTGCGGCTGGTGTGCCTGGGGGGGCCAGGATGGGCGTGGCCGCTGCATGGAGGGTGGACTCAGCG
GCCCCCGTGATGGGCTGACATGTGGGCGTCCGGGGGCCTCCTGGGCCTTCCTGTCCTGCCCCCCTGAGGA
CGAGTGTGCAAACGGGCACCACGACTGCAACGAGACGCAGAATTGCCACGACCAGCCCCACGGCTATGAG
TGCAGCTGCAAGACCGGCTATACCATGGACAACATGACAGGGCTGTGCCGCCCTGTGTGCGCCCAGGGCT
GCGTGAACGGCTCATGTGTGGAGCCCGACCACTGCCGCTGCCACTTTGGCTTTGTGGGCCGCAACTGCTC
CACGGAATGCCGCTGCAACCGCCACAGTGAATGCGCTGGTGTTGGGGCGCGTCACCACTGCTTGCTCTGC
CGCAACCACACCAAGGGCAGCCACTGTGAGCAGTGCCTCCCGCTGTTTGTGGGTTCAGCTGTCGGAGGCG
GGACCTGCCGGCCCTGCCACGCCTTTTGTCGTGGAAATAGCCACATCTGCATCTCCAGGAAGGAGTTACA
AATGTCCAAGGGAGAGCCAAAGAACTACTCACTGGACCCAGAGGAGATTGAAAACTGGGTGACAGAGGGT
CCTAGTGAAGACGAGGCCGTGTGCGTGAACTGCCAGAATAACAGCTATGGGGAGAAATGCGAGAGCTGCC
TGCAGGGCTACTTCCTCCTGGACGGGAAGTGCACCAAATGCCAGTGTAATGGCCACGCGGACACATGTAA
CGAGCAGGATGGGACGGGCTGTCCATGTCAGAATAACACAGAGACGGGCACATGCCAGGGCAGCTCCCCC
AGTGACCGTCGAGACTGCTACAAGTACCAGTGCGCCAAGTGCCGGGAATCATTTCACGGGAGTCCGCTGG
GCGGCCAGCAGTGCTACCGCCTCATCTCGGTGGAGCAGGAGTGCTGCCTGGACCCCACGTCCCAGACCAA
CTGCTTCCATGAGCCCAAACGCCGGGCGCTAGGCCCCGGCCGCACTGTCCTCTTTGGCGTGCAGCCCAAA
TTCACCAACGTGGACATCCGCCTGACGCTGGACGTGACCTTCGGGGCCGTGGACCTCTATGTCTCCACCT
CCTATGACACCTTCGTGGTCCGTGTGGCCCCTGACACTGGCGTCCATACTGTACACATCCAGCCACCCCC
AGCCCCACCACCTCCACCACCCCCTGCAGATGGTGGGCCCCGGGGGGCTGGGGATCCAGGAGGAGCAGGG
GCCAGCAGTGGGCCGGGCGCCCCAGCAGAGCCACGGGTACGGGAGGTATGGCCGCGGGGCCTGATTACCT
ACGTGACGGTGACGGAGCCGTCGGCAGTGCTGGTGGTCCGCGGCGTGCGGGACCGGCTGGTCATCACCTA
CCCACACGAGCACCATGCCCTCAAGTCGAGCCGCTTCTACCTGCTGCTGCTGGGCGTGGGAGACCCAAGT
GGGCCCGGCGCCAACGGCTCAGCCGACTCGCAGGGCCTGCTCTTCTTCCGGCAGGACCAGGCCCACATTG
ACCTGTTTGTCTTCTTCTCCGTCTTCTTCTCCTGCTTCTTCCTCTTCCTCTCACTCTGTGTGCTCCTCTG
GAAGGCCAAGCAGGCTCTGGACCAGCGGCAGGAGCAGCGCCGGCACTTGCAGGAGATGACCAAGATGGCC
AGCCGCCCCTTCGCCAAGGTCACCGTCTGCTTCCCACCTGACCCTACTGCCCCGGCCTCCGCCTGGAAGC
CGGCTGGGCTCCCACCTCCCGCCTTCCGCCGCTCTGAGCCCTTCCTGGCACCCCTGCTGCTGACAGGGGC
CGGTGGGCCCTGGGGACCCATGGGAGGGGGCTGCTGCCCACCAGCCATCCCCGCCACCACTGCTGGGCTG
CGAGCTGGGCCCATCACTCTCGAGCCCACAGAAGATGGCATGGCTGGCGTGGCCACACTGCTGCTCCAGC
TGCCTGGCGGGCCCCATGCACCCAACGGCGCCTGCCTGGGGTCAGCCCTCGTCACACTGCGGCACAGGCT
GCACGAGTACTGTGGGGGTGGTGGGGGTGCTGGGGGCAGTGGGCATGGGACTGGTGCGGGCCGGAAGGGA
CTGTTGAGCCAGGACAACCTCACCAGCATGTCCCTCTGACATGCCCAGG

In a search of public sequence databases, the NOV7 nucleic acid sequence, located on chromsome 19 has 5224 of 5224 bases (100%) identical to a gb:GENBANK-ID:AB01541|acc:AB011541.1 mRNA from Homo sapiens (Homo sapiens mRNA for MEGF8, partial cds). Public nucleotide databases include all GenBank databases and the GeneSeq patent database.

The disclosed NOV7 polypeptide (SEQ ID NO:16) encoded by SEQ ID NO:15 has 2854 amino acid residues and is presented in Table 7B using the one-letter amino acid code. Signal P, Psort and/or Hydropathy results predict that NOV7 has a signal peptide and is likely to be localized at the plasma membrane with a certainty of 0.4600. The most likely cleavage site for a NOV7 peptide is between amino acids 27 and 28.

TABLE 7B
Encoded NOV7 protein sequence.

| | |
|---|---|
| MALGKVLAMALVLALAVLGSLSPGARAGDCKGQRQVLREAPGFVTDGAGNYSVNGNCEWL | (SEQ ID NO:16) |
| IEAPSPQHRILLDFLFLDTECTYDYLFVYDGDSPRGPLLASLSGSTRPPPIEASSGKMLL | |
| HLFSDANYNLLGFNASFRFSLCPGGCQSHGQCQPPGVCACEPGWGGPDCGLQECSAYCGS | |
| HGTCASPLGPCRCEPGFLGRACDLHLWENQGAGWWHNVSARDPAFSARIGAAGAFLSPPG | |
| LLAVFGGQDLNNALGDLVLYNFSANTWESWDLSPAPAARHSHVAVAWAGSLVLMGGELAD | |
| GSLTNDVWAFSPLGRGHWELLAPPASSSSGPPGLAGHAAALVDDVWLYVSGGRTPHDLFS | |
| SGLFRFRLDSTSGGYWEQVIPAGGRPPAATCHSMVFHAPSRALLVHGGHRPSTARFSVRV | |
| NSTELFHVDRHVWTTLKGRDGLQGPRERAFHTASVLGNYMVVYGGNVHTHYQEEKCYEDG | |
| IFFYHLGCHQWVSGAELAPPGTPEGRAAPPSGRYSHVAAVLGGSVLLVAGGYSGRPRGDL | |
| MAYKVPPFVFQAPAPDYHLDYCSMYTDHSVCSRDPECSWCQGACQAAPPPGTPLGACPAA | |
| SCLGLGRLLGDCQACLAFSSPTAPPRGPGTLGWCVHNESCLPRPEQARCRGEQISGTVGW | |
| WGPAPVFVTSLEACVTQSFLPGLHLLTFQQPPNTSQPDKVSIVRSTTITLTPSAETDVSL | |
| VYRGFIYPMLPGGPGGPGAEDVAVWTRAQRLHVLARMARGPDTENMEEVGRWVAHQEKET | |
| RRLQRPGSARLFPLPGRDHKYAVEIQGQLNGSAGPGHSELTLLWDRTGVPGGSEISFFFL | |
| EPYRSSSCTSYSSCLGCLADQGCGWCLTSATCHLRQGGAHCGDDGAGGSLLVLVPTLCPL | |
| CEEHRDCHACTQDPFCEWHQSTSRKGDAACSRRGRGRGALKSPEECPPLCSQRLTCEDCL | |
| ANSSQCAWCQSTHTCFLFAAYLARYPHGGCRGWDDSVHSEPRCRSCDGFLTCHECLQSHE | |
| CGWCGNEDNPTLGRCLQGDFSGPLGGGNCSLWVGEGLGLPVALPARWAYARCPDVDECRL | |
| GLARCHPRATCLNTPLSYECHCQRGYQGDGISHCNRTDGISHCNRTCLEDCGHGVCSGPP | |
| DFTCVCDLGWTSDLPPPTPAPGPPAPRCSRDCGCSFHSHCRKRGPGFCDECQDWTWGEHC | |
| ERCRPGSFGNATGSRGCRPCQCNGHGDPRRGHCDNLSGLCFCQDHTEGAHCQLCSPGYYG | |
| DPRAGGSCFRECGGRALLTNVSSVALGSRRVGGLLPPGGGAARAGPGLSYCVWVVSATEE | |
| LQPCAPGTLCPPLTLTFSPDSSTPCTLSYVLAFDGFPRFLDTGVVQSDRSLIAAFCGQRR | |
| DRPLTVQALSGLLVLHWEANGSSSWGFNASVGSARCGSGGPGSCPVPQECVPQDGAAGAG | |
| LCRCPQGWAGPHCRMALCPENCNAHTGAGTCNQSLGVCICAEGFGGPDCATKLDGGQLVW | |
| ETLMDSRLSADTASRFLHRLGHTMVDGPDATLWMFGGLGLPQGLLGNLYRYSVSERRWTQ | |
| MLAGAEDGGPGPSPRSFHAAAYVPAGRGAMYLLGGLTAGGVTRDFWVLNLTTLQWRQEKA | |
| PQTVELPAVAGHTLTARRGLSLLLVGGYSPENGFNQQLLEYQLATGTWVSGAQSGTPPTG | |
| LYGHSAVYHEATDSLYVFGGFRFHVELAAPSPELYSLHCPDRTWSLLAPSQGAKRDRMRN | |
| VRGSSRGLGQVPGEQPGSWGFREVRKKMALWAALAGTGGFLEEISPHLKEPRPRLFHASA | |
| LLGDTMVVLGGRSDPDEFSSDVLLYQVNCNAWLLPDLTRSASVGPPMEESVAHAVAAVGS | |
| RLYISGGFGGVALGRLLALTLPPDPCRLLSSPEACNQSGACTWCHGACLSGDQAHRLGCG | |
| GSPCSPMPRSPEECRRLRTCSECLARHPRTLQPGDGEASTPRCKWCTNCPEGACIGRNGS | |
| CTSENDCRINQREVFWAGNCSEAACGAADCEQCTREGKCMWTRQFKRTGETRRILSVQPT | |
| YDWTCFSHSLLNVSPMPVESSPPLPCPTPCHLLPNCTSCLDSKGADGGWQHCVWSSSLQQ | |
| CLSPSYLPLRCMAGGCGRLLRGPESCSLGCAQATQCALCLRRPHCGWCAWGGQDGGGRCM | |
| EGGLSGPRDGLTCGRPGASWAFLSCPPEDECANGHHDCNETQNCHDQPHGYECSCKTGYT | |
| MDNMTGLCRPVCAQGCVNGSCVEPDHCRCHFGFVGRNCSTECRCNRHSECAGVGARDHCL | |
| LCRNHTKGSHCEQCLPLFVGSAVGGGTCRPCHAFCRGNSHICISRKELQMSKGEPKKYSL | |
| DPEEIENWVTEGPSEDEAVCVNCQNNSYGEKCESCLQGYFLLDGKCTKCQCNGHADTCNE | |
| QDGTGCPCQNNTETGTCQGSSPSDRRDCYKYQCAKCRESFHGSPLGGQQCYRLISVEQEC | |
| CLDPTSQTNCFHEPKRRALGPGRTVLFGVQPKFTNVDIRLTLDVTFGAVDLYVSTSYDTF | |
| VVRVAPDTGVHTVHIQPPPAPPPPPPPADGGPRGAGDPGGAGASSGPGAPAEPRVREVWP | |
| RGLITYVTVTEPSAVLVVRGVRDRLVITYPHEHHALKSSRFYLLLLGVGDPSGPGANGSA | |
| DSQGLLFFRQDQAHIDLFVFFSVFFSCFFLFLSLCVLLWKAKQALDQRQEQRRHLQEMTK | |
| MASRPFAKVTVCFPPDPTAPASAWKPAGLPPPAFRRSEPFLAPLLLTGAGGPWGPMGGGC | |
| CPPAIPATTAGLRAGPITLEPTEDGMAGVATLLLQLPGGPHAPNGACLGSALVTLRHRLH | |
| EYCGGGGGAGGSGHGTGAGRKGLLSQDNLTSMSL | |

A search of sequence databases reveals that the NOV7 amino acid sequence has 1737 of 1737 amino acid residues (100%) identical to, and 1737 of 1737 amino acid residues (100%) similar to, the 1737 amino acid residue ptnr:SPTREMBL-ACC:O75097 protein from Homo sapiens (Human) (MEGF8). Public amino acid databases include the GenBank databases, SwissProt, PDB and PIR.

NOV7 is expressed in at least kidney, nervous system, brain, lung. This information was derived by determining the tissue sources of the sequences that were included in the invention including but not limited to SeqCalling sources, Public EST sources, Literature sources, and/or RACE sources.

The disclosed NOV7 polypeptide has homology to the amino acid sequences shown in the BLASTP data listed in Table 7C.

TABLE 7C
BLAST results for NOV7

| Gene Index/ | | Length | Identity | Positives | |
|---|---|---|---|---|---|
| Identifier | Protein/Organism | (aa) | (%) | (%) | Expect |
| gi\|7513144\|pir\|\|T00209 | MEGF8 protein - | 1737 | 1737/1737 | 1737/1737 | 0.0 |
| | human (fragment) | | (100%) | (100%) | |
| gi\|14756108\|ref\|XP_ | EGF-like- | 1214 | 1171/1173 | 1171/1173 | 0.0 |
| 029883.1\| | domain, multiple 4 | | (99%) | (99%) | |
| (XM_029883) | [Homo sapiens] | | | | |
| gi\|6681364\|dbj\|BAA88689.1\| | MEGF8 [Rattus | 874 | 836/874 | 850/874 | 0.0 |
| (AB011534) | norvegicus] | | (95%) | (96%) | |
| gi\|10728654\|gb\|AAF52597.2\| | CG7466 gene | 2820 | 786/2379 | 1107/2379 | 0.0 |
| (AE003619) | product | | (33%) | (46%) | |
| | [Drosophila | | | | |
| | melanogaster] | | | | |
| gi\|17862106\|gb\|AAL39530.1\| | LD09511p | 779 | 192/466 | 263/466 | 3e−94 |
| (AY069385) | [Drosophila | | (41%) | (56%) | |
| | melanogaster] | | | | |

Tables 7D-F list the domain descriptions from DOMAIN analysis results against NOV7. This indicates that the NOV7 sequence has properties similar to those of other proteins known to contain this domain.

TABLE 7D
Domain Analysis of NOV7

gnl|Smart|smart00042, CUB, Domain first found in Clr, Cls, uEGF, and
bone morphogenetic protein.; This domain is found mostly among
developmentally-regulated proteins. Spermadhesins contain only this
domain.
CD-Length = 114 residues, 82.5% aligned
Score = 84.0 bits (206), Expect = 1e−16

TABLE 7E
Domain Analysis of NOV7

gnl|Pfam|pfam00053, laminin_EGF, Laminin EGF-like
(Domains III and V). This family is like pfam00008 but has 8
conserved cysteines instead of 6.
CD-Length = 49 residues, 87.8% aligned
Score = 47.4 bits (111), Expect = 1e−05

TABLE 7F
Domain Analysis of NOV7

gnl|Smart|smart00179, EGF_CA, Calcium-binding EGF-like domain
CD-Length = 41 residues, 90.2% aligned
Score = 38.5 bits (88), Expect = 0.005

The domain that characterizes epidermal growth factor (EGF) consists of approximately 50 amino acids, and has been shown to be present in a more or less conserved form in a large number of other, mostly animal proteins. EGF-like domains are believed to play a critical role in a number of extracellular events, including cell adhesion and receptor-ligand interactions. Proteins with EGF-like domains often consist of more than 1,000 amino acids, have multiple copies of the EGF-like domain, and contain additional domains known to be involved in specific protein-protein interactions. The list of proteins currently known to contain one or more copies of an EGF-like pattern is large and varied. The functional significance of EGF domains in what appear to be unrelated proteins is not yet clear. However, a common feature is that these repeats are found in the extracellular domain of membrane-bound proteins or in proteins known to be secreted (exception: prostaglandin G/H synthase). The EGF domain includes six cysteine residues which have been shown (in EGF) to be involved in 3 disulfide bonds. The main structure is a two-stranded beta-sheet followed by a loop to a C-terminal short two-stranded sheet. Subdomains between the conserved cysteines vary in length.

To identify proteins containing EGF-like domains, Nakayama et al. (1998) searched a database of long cDNA sequences randomly selected from a human brain cDNA library for those that encode an EGF-like motif. They identified several partial cDNAs encoding novel proteins with multiple EGF-like domains, such as EGFL4, which they named MEGF8. The predicted partial EGFL4 protein has a laminin-type EGF-like domain, 5 EGF-like domains, and a transmembrane domain. Using a radiation hybrid mapping panel, Nakayama et al. (1998) mapped the EGFL4 gene to 19q12.

The disclosed NOV7 nucleic acid of the invention encoding a MEGF8-like protein includes the nucleic acid whose sequence is provided in Table 7A or a fragment thereof. The invention also includes a mutant or variant nucleic acid any of whose bases may be changed from the corresponding base shown in Table 7A while still encoding a protein that maintains its MEGF8-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 0 percent of the bases may be so changed.

The disclosed NOV7 protein of the invention includes the MEGF8-like protein whose sequence is provided in Table 7B. The invention also includes a mutant or variant protein any of whose residues may be changed from the corresponding residue shown in Table 7B while still encoding a protein that maintains its MEGF8-like activities and physiological functions, or a functional fragment thereof. In the mutant or variant protein, up to about 0 percent of the residues may be so changed.

The invention further encompasses antibodies and antibody fragments, Such as F_{ab} or (F_{ab})₂, that bind immunospecifically to any of the proteins of the invention.

The above defined information for this invention suggests that this MEGF8-like protein (NOV7) may function as a member of a “MEGF8 family”. Therefore, the NOV7 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV7 nucleic acids and proteins of the invention are useful in potential therapeutic applications implicated in diseases including but not limited to various pathologies and disorders as indicated below. For example, a cDNA encoding the MEGF8-like protein (NOV7) may be useful in gene therapy, and the MEGF8-like protein (NOV7) may be useful when administered to a subject in need thereof. By way of nonlimiting example, the compositions of the present invention will have efficacy for treatment of patients suffering from Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia telangiectasia, leukodystrophies, behavioral disorders, addiction, anxiety, pain, neuroprotection, multiple sclerosis, myasthenia gravis, systemic lupus erythematosus, autoimmune disease, asthma, emphysema, scleroderma, allergy, ARDS, diabetes, renal artery stenosis, interstitial nephritis, glomerulonephritis, polycystic kidney disease, renal tubular acidosis, IgA nephropathy, or other pathologies or conditions. The NOV7 nucleic acid encoding the MEGF8-like protein of the invention, or fragments thereof, may further be useful in diagnostic applications, wherein the presence or amount of the nucleic acid or the protein are to be assessed.

NOV7 nucleic acids and polypeptides are further useful in the generation of antibodies that bind immuno-specifically to the novel NOV7 substances for use in therapeutic or diagnostic methods. These antibodies may be generated according to methods known in the art, using prediction from hydrophobicity charts, as described in the “Anti-NOVX Antibodies” section below. The disclosed NOV7 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV8

NOV8 includes two protocadherin-like proteins disclosed below. The disclosed sequences have been named NOV8a and NOV8b.

NOV8a

A disclosed NOV8a nucleic acid of 6006 nucleotides (also referred to as CG57542-01) encoding a protocadherin-like protein is shown in Table 8A. Putative untranslated regions upstream and/or downstream from the coding region, if any, are underlined, and the start and stop codons are in bold letters.

TABLE 8A
NOV8a nucleotide sequence.

| | |
|---|---|
| CAAATGTTTCTTTCTACCTTTTCTTATTTCAGATCAGCTTTGAGTGAACTTTGACAGAAG | (SEQ ID NO:17) |
| ATGTTTCGACAGTTTTATCTCTGGACATGTTTAGCTTCAGGGATCATCCTGGGCTCTCTC | |
| TTTGAAATCTGCTTGGGCCAGTATGATGATGGTAAGGATTGCAAACTAGCTAGGGGAGGA | |
| CCACCAGCTACCATAGTTGCTATTGATGAAGAAAGTCGGAATGGTGCAGGTACAATTCTG | |
| GTGGACAACATGCTGATCAAAGGGACTGCTGGAGGACCAGACCCCACCATAGAACTTTCT | |
| TTAAAGGATAATGTGGATTACTGGGTGTTGATGGATCCTGTTAAGCAAATGCTTTTCCTG | |
| AACAGCACCGGAAGAGTTCTGGATAGAGATCCACCGATGAACATACACTCCATTGTGGTG | |
| CAGGTCCAGTGCATCAACAAAAAAGTGGGCACTATTATCTACCATGAAGTGCGAATAGTG | |
| GTGAGAGACAGGAATGACAACTCACCCACTTTCAAGCATGAAAGCTACTATGCCACAGTG | |
| AATGAGCTCACTCCAGTTGGTACCACAATATTCACAGGATTTTCAGGAGACAATGGAGCT | |
| ACAGATATAGATGATGGACCAAATGGACAGATAGAGTATGTTATTCAGTATAATCCAGAT | |
| GATCCGACATCCAATGACACCTTTGAAATTCCCCTAATGTTGACTGGAAATATAGTGTTA | |
| AGGAAGAGGCTCAACTATGAAGATAAGACTCGCTACTTTGTCATAATCCAAGCTAATGAC | |
| CGTGCCCAAAATCTGAATGAGAGGCGAACCACCACCACCACTCTCACAGTGGATGTTCTG | |
| GATCGAGATGACTTGGGTCCAATGTTTCTTCCTTGTGTCCTTGTGCCAAACACTCGTGAT | |
| TGCCGTCCACTCACTTATCAAGCTGCCATACCTGAGTTGAGAACTCCGGAAGAACTGAAC | |
| CCCATTATTGTTACGCCACCAATCCAAGCCATTGATCAGGACCGGAATATTCAACCGCCA | |
| TCAGATAGGCCAGGAATCCTCTATTCCATCCTTGTTGGTGGGACTCCTGAGGATTACCCA | |
| CGATTTTTCCATATGCATCCTAGGACAGCAGAACTTAGTCTCCTGGAGCCAGTAAACAGA | |
| GACTTTCACCAGAAATTTGATTTGGTTATTAAGGCTGAACAAGACAATGGTCATCCTCTT | |
| CCTGCCTTTGCCGGTCTACACATTGAAATACTGGATGAAAACAATCAAAGTCCATATTTT | |
| ACAATGCCCAGTTATCAAGGCTATATCCTGGAATCTGCCCCAGTGGGAGCAACCATTTCG | |
| GACAGTCTCAATTTGACCTCACCTTTAAGAATAGTAGCTCTGGACAAGGACATAGAAGAT | |
| ACAAAAGACCCAGAGCTTCACCTTTTTCTGAATGACTACACCTCAGTCTTCACCGTCACA | |
| CAGACTGGTATTACTCGCTACCTCACCTTACTTCAACCAGTGGACAGGGAAGAACAGCAA | |
| ACTTACACCTTTTCGATAACAGCATTTGATGGTGTACAAGAAAGTGAGCCAGTCATCGTC | |
| AATATTCAAGTGATGGATGCAAATGATAACACGCCAACCTTCCCTGAAATATCCTATGAT | |
| GTGTATGTTTATACAGACATGAGACCTGGGGACAGTGTCATACAGCTCACTGCAGTCGAC | |
| GCAGACGAAGGGTCAAATGGGGAGATCACATATGAAATCCTTGTTGGGGCTCAGGGAGAC | |
| TTCATCATCAATAAAACAACAGGGCTTATCACCATCGCTCCAGGGGTGGAAATGATAGTC | |
| GGGCGGACTTACGCACTCACGGTCCAAGCAGCGGATAATGCTCCTCCTGCAGAGCGAAGG | |
| AACTCCATCTGCACTGTGTATATTGAAGTGCTTCCACCAAATAATCAAAGCCCTCCTCGC | |
| TTCCCACAGCTGATGTATAGCCTTGAAATTAGTGAAGCCATGAGGGTTGGTGCTGTTTTA | |
| TTAAATCTACAGGCAACTGATCGAGAGGGAGACTCAATAACATATGCCATTGAGAATGGA | |
| GATCCTCAGAGAGTTTTTAATCTTTCAGAAACCACGGGGATTCTAACCTTAGGGAAAGCA | |
| CTGGACAGGGAAAGCACTGATCGCTACATTCTGATCATCACAGCTTCAGATGGCAGGCCA | |
| GATGGGACCTCAACTGCCACAGTAAACATAATGGTGACAGATGTCAATGACAATGCTCCA | |
| GTGTTTGATCCTTATCTGCCAAGAAATTTATCTGTGGTGGAAGAAGAAGCCAATGCCTTT | |
| GTGGGTCAAGTAAAAGCAACAGACCCTGATGCTGGAATAAATGGTCAAGTGCACTACAGT | |
| TTGGGTAACTTTAATAATCTTTTTCGTATCACATCCAATGGGAGCATTTACACAGCAGTG | |
| AAGCTTAACAGAGAAGTCAGGGACTACTATGAACTTGTTGTTGTGGCAACAGATGGAGCA | |
| GTACACCCTCGTCATTCAACTCTAACCTTGGCCATCAAGGTTTTGGACATTGATGATAAC | |
| AGTCCTGTGTTCACCAATTCAACATACACTGTCCTTGTTGAAGAGAATTTGCCAGCTGGG | |
| ACTACCATCCTTCAAATAGAGGCCAAAGATGTCGACCTTGGAGCAAATGTGTCTTACCGG | |
| ATAAGAAGCCCAGAAGTGAAGCACTTTTTTGCACTACATCCATTTACAGGAGAACTATCG | |
| CTTTTAAGGAGTTTAGATTATGAGGCATTTCCAGACCAAGAAGCAAGTATCACTTTTCTG | |
| GTAGAGGCCTTTGATATTTATGGAACAATGCCACCTGGTATTGCTACTGTCACAGTGATT | |
| GTAAAGGATATGAATGATTATCCTCCTGTCTTTAGTAAACGAATATACAAAGGGATGGTG | |
| GCTCCGGATGCAGTCAAGGGTACACCTATCACAACAGTTTATGCTGAAGATGCAGACCCT | |
| CCTGGATTACCTGCAAGTCGTGTGAGGTATAGAGTAGATGATGTACAGTTTCCTTACCCT | |
| GCCAGTATTTTTGAAGTGGAAGAAGATTCTGGAAGAGTAATAACACGAGTCAATCTTAAT | |
| GAAGAACCTACAACAATTTTTAAGTTGGTGGTGGTTGCTTTTGATGATGGGGAGCCTGTG | |
| ATGTCCAGCAGTGCCACAGTGAAGATTCTTGTCTTACATCCTGGTGAGATCCCACGCTTC | |
| ACACAGGAGGAATATAGGCCTCCTCCAGTAAGTGAACTTGCCACCAAAGGGACCATGGTT | |
| GGTGTAATTTCTGCTGCTGCCATTAATCAAAGTATTGTGTACTCCATTCTTTCAGGAAAT | |
| GAAGAAGATACATTTGGAATTAATAACATCACAGGTGTTATCTATGTGAATGGACCTCTG | |
| GATTATGAGACCACGACAAGCTATGTACTTCGAGTCCAAGCTGATTCCCTGGAAGTGGTC | |
| CTTGCCAATCTCCGAGTTCCTTCAAAAAGTAATACAGCTAAAGTATACATTGAGATTCAG | |
| GATGAAAATAATCATCCCCCAGTGTTTCAGAAAAAATTCTACATCGGAGGTGTATCTGAA | |
| GATGCAAGAATGTTTACTTCTGTACTCAGAGTGAAGGCTACTGATAAAGATACTCCCAAT | |
| TATAGTGTCATGGCCTACAGACTCATAATACCACCAATTAAACAGGGAAAAGAAGGATTT | |
| GTAGTGGAAACATATACAGGGCTTATCAAAACTGCTATGCTCTTCCATAATATGAGGAGA | |
| TCCTACTTCAAGTTTCAAGTTATTGCAACTGACGACTATGGGAAGGGACTGAGCGGCAAA | |
| GCCGATGTACTCGTAAGTGTCTCCGTGGTCAATCAGCTGGATATGCAAGTCATTGTTTCC | |
| AATGTGCCTCCTACTCTAGTGGAAAAAAAGATAGAAGATCTTACAGAAATCTTGGATCGC | |
| TATGTTCAGGAACAAATTCCTGGTGCCAAGGTCGTAGTGGAGTCCATTGGAGCTCGCCGG | |
| CATGGAGATGCCTTTTCCCTAGAAGATTACACCAAATGTGACTTGACTGTCTATGCAATT | |
| GACCCCCAAACCAACAGAGCCATCGATAGAAATGAGCTTTTTAAGTTTTTGGATGGCAAA | |
| CTACTTGATATCAATAAAGACTTTCAGCCGTATTATGGGGAAGGAGGACGCATTCTGGAG | |
| ATCCGGACTCCAGAGGCAGTGACCAGCATTAAAAAGAGAGGAGAAAGTCTAGGATACACA | |
| GAAGGGGCCTTGTTGGCTCTGGCCTTCATCATCATCCTCTGCTGCATTCCTGCCATCTTG | |
| GTGGTTTTGGTCAGCTACAGACAGTTTAAAGTACGTCAAGCTGAGTGTACACGTCAAGCT | |
| GAGTGTACAAAGACTGCACGAATTCAGGCCGCATTACCCGCGGCTAAACCAGCAGTGCCG | |
| GCTCCTGCACCAGTGGCAGCGCCCCCGCCGCCGCCGCCGCCTCCGCCAGGTGCGCATCTC | |
| TATGAAGAACTTGGAGACAGCTCAATGTCTTTTCTTTCAAGTCTTTTCCTTCTCTACCAT | |
| TTTCAACAAAGCAGGGGAAATAACTCAGTCTCAGAAGACAGGAAACATCAACAAGTTGTG | |
| ATGCCCTTTTCTTCCAATACTATTGAGGCTCACAAGTCAGCTCATGTAGACGGATCACTT | |
| AAGAGCAACAAACTGAAGTCTGCAAGAAAATTCACATTTCTATCTGATGAGGATGACTTA | |
| AGTGCCCATAATCCCCTTTATAAGGAAAACATAAGTCAAGTATCAACAAATTCAGACATT | |
| TCACAGAGAACAGATTTTGTAGACCCATTTTCACCCAAAATACAAGCCAAGAGTAAGTCT | |
| CTGAGGGGCCCAAGAGAAAAGATTCAGAGGCTGTGGAGTCAGTCAGTCAGCTTACCCAGG | |
| AGGCTGATGAGGAAAGTTCCAAATAGACCAGAGATCATAGATCTGCAGCAGTGGCAAGGC | |
| ACCAGGCAGAAAGCTGAAAATGAAAACACTGGAATCTGTACAAACAAAAGAGGTAGCAGC | |
| AATCCATTGCTTACAACTGAAGAGGCAAATTTGACAGAGAAAGAGGAAATAAGGCAAGGT | |
| GAAACACTGATGATAGAAGGAACAGAACAGTTGAAATCTCTCTCTTCAGACTCTTCATTT | |
| TGCTTTCCCAGGCCTCACTTCTCATTCTCCACTTTGCCAACTGTTTCAAGAACTGTGGAA | |
| CTCAAATCAGAACCTAATGTCATCAGTTCTCCTGCTGAGTGTTCCTTGGAACTTTCTCCT | |
| TCAAGGCCTTGTGTTTTACATTCTTCACTCTCTAGGAGAGAGACACCTATTTGTATGTTA | |
| CCTATTGAAACCGAAAGAAATATTTTTGAAAATTTTGCCCATCCACCAAACATCTCTCCT | |
| TCTGCCTGTCCCCTTCCCCCTCCTCCTCCTATTTCTCCTCCTTCTCCTCCTCCTGCTCCT | |
| GCTCCTCTTGCTCCTCCTCCTGACATTTCTCCTTTTTCTCTTTTTTGTCCTCCTCCCTCT | |
| CCTCCTTCTATCCCTCTTCCTCTTCCTCCTCCTACATTTTTTCCACTTTCCGTTTCAACG | |
| TCTGGTCCCCCAACACCACCTCTTCTACCTCCATTTCCAACTCCTCTTCCTCCACCACCT | |
| CCTTCTATTCCTTGCCCTCCACCTCCTTCAGCTTCATTTCTGTCCACAGAGTGTGTCTGT | |
| ATAACAGGTGTTAAATGCACGACCAACTTGATGCCTGCCGAGAAAATTAAGTCCTCTATG | |
| ACACAGCTATCAACAACGACAGTGTGTAAAACAGACCCTCAGACAGAACCAAAAGGCATC | |
| CTCAGACACGTTAAAAACTTAGCAGAACTTGAAAAATCAGTAGCTAACATGTACAGTCAA | |
| ATAGAAAAAAACTATCTACGCACAAATGTTTCAGAACTTCAAACTATGTGCCCTTCAGAA | |
| GTAACAAATATGGAAATCACATCTGAACAAAACAAGGGGAGTTTGAACAATATTGTCGAG | |
| GGAACTGAAAAACAATCTCACAGTCAATCTACTTCACTGTAATGTTGCTTTTCTTATTTT | |
| AGTCGG | |

In a search of public sequence databases, the NOV8a nucleic acid sequence, located on chromsome 10 has 557 of 955 bases (58%) identical to a GENBANK-ID:AF169693|acc:AF169693.1 mRNA from Homo sapiens (Homo sapiens protocadherin 13 (PCDH13) mRNA, partial cds). Public nucleotide databases include all GenBank databases and the GeneSeq patent database.

The disclosed NOV8a polypeptide (SEQ ID NO:18) encoded by SEQ ID NO:17 has 1973 amino acid residues and is presented in Table 8B using the one-letter amino acid code. Signal P, Psort and/or Hydropathy results predict that NOV8 has a signal peptide and is likely to be localized extracellularly with a certainty of 0.6760. The most likely cleavage point is between residues 26 and 27.

TABLE 8B
Encoded NOV8a protein sequence.

| | |
|---|---|
| MFRQFYLWTCLASGIILGSLFEICLGQYDDGKDCKLARGGPPATIVAIDEESRNGAGTIL | (SEQ ID NO:18) |
| VDNMLIKGTAGGPDPTIELSLKDNVDYWVLMDPVKQMLFLNSTGRVLDRDPPMNIHSIVV | |
| QVQCINKKVGTIIYHEVRIVVRDRNDNSPTFKHESYYATVNELTPVGTTIFTGFSGDNGA | |
| TDIDDGPNGQIEYVIQYNPDDPTSNDTFEIPLMLTGNIVLRKRLNYEDKTRYFVIIQAND | |
| RAQNLNERRTTTTTLTVDVLDGDDLGPMFLPCVLVPNTRDCRPLTYQAAIPELRTPEELN | |
| PIIVTPPIQAIDQDRNIQPPSDRPGILYSILVGGTPEDYPRFFHMHPRTAELSLLEPVNR | |
| DFHQKFDLVIKAEQDNGHPLPAFASLHIEILDENNQSPYFTMPSYQGYILESAPVGATIS | |
| DSLNLTSPLRIVALDKDIEDTKDPELHLFLNDYTSVFTVTQTGITRYLTLLQPVDREEQQ | |
| TYTFSITAFDGVQESEPVIVNIQVMDANDNTPTFPEISYDVYVYTDMRPGDSVIQLTAVD | |
| ADEGSNGEITYEILVGAQGDFIINKTTGLITIAPGVEMIVGRTYALTVQAADNAPPAERR | |
| NSICTVYIEVLPPNNQSPPRFPQLMYSLEISEAMRVGAVLLNLQATDREGDSITYAIENG | |
| DPQRVFNLSETTGILTLGKALDRESTDRYILIITASDGRPDGTSTATVNIMVTDVNDNAP | |
| VFDPYLPRNLSVVEEEANAFVGQVKATDPDAGINGQVHYSLGNFNNLFRITSNGSIYTAV | |
| KLNREVRDYYELVVVATDGAVHPRHSTLTLAIKVLDIDDNSPVFTNSTYTVLVEENLPAG | |
| TTILQIEAKDVDLGANVSYRIRSPEVKHFFALHPFTGELSLLRSLDYEAFPDQEASITFL | |
| VEAFDIYGTMPPGIATVTVIVKDMNDYPPVFSKRIYKGMVAPDAVKGTPITTVYAEDADP | |
| PGLPASRVRYRVDDVQFPYPASIFEVEEDSGRVITRVNLNEEPTTIFKLVVVAFDDGEPV | |
| MSSSATVKILVLHPGEIPRFTQEEYRPPPVSELATKGTMVGVISAAAINQSIVYSIVSGN | |
| EEDTFGINNITGVIYVNGPLDYETRTSYVLRVQADSLEVVLANLRVPSKSNTAKVYIEIQ | |
| DENNHPPVFQKKFYIGGVSEDARMFTSVLRVKATDKDTGNYSVMAYRLIIPPIKEGKEGF | |
| VVETYTGLIKTAMLFHNMRRSYFKFQVIATDDYGKGLSGKADVLVSVSVVNQLDMQVIVS | |
| NVPPTLVEKKIEDLTEILDRYVQEQIPGAKVVVESIGARRHGDAFSLEDYTKCDLTVYAI | |
| DPQTNRAIDRNELFKFLDGKLLDINKDFQPYYGEGGRILEIRTPEAVTSIKKRGESLGYT | |
| EGALLALAFIIILCCIPAILVVLVSYRQFKVRQAECTRQAECTKTARIQAALPAAKPAVP | |
| APAPVAAPPPPPPPPPGAHLYEELGDSSMSFLSSLFLLYHFQQSRGNNSVSEDRKHQQVV | |
| MPFSSNTIEAHKSAHVDGSLKSNKLKSARKFTFLSDEDDLSAHNPLYKENISQVSTNSDI | |
| SQRTDFVDPFSPKIQAKSKSLRGPREKIQRLWSQSVSLPRRLMRKVPNRPEIIDLQQWQG | |
| TRQKAENENTGICTNKRGSSNPLLTTEEANLTEKEEIRQGETLMIEGTEQLKSLSSDSSF | |
| CFPRPHFSFSTLPTVSRTVELKSEPNVISSPAECSLELSPSRPCVLHSSLSRRETPICML | |
| PIETERNIFENFAHPPNISPSACPLPPPPPISPPSPPPAPAPLAPPPDISPFSLFCPPPS | |
| PPSIPLPLPPPTFFPLSVSTSGPPTPPLLPPFPTPLPPPPPSIPCPPPPSASFLSTECVC | |
| ITGVKCTTNLMPAEKIKSSMTQLSTTTVCKTDPQREPKGILRHVKNLAELEKSVANMYSQ | |
| IEKNYLRTNVSELQTMCPSEVTNMEITSEQNKGSLNNIVEGTEKQSHSQSTSL | |

A search of sequence databases reveals that the NOV8a amino acid sequence has 1580 of 1846 amino acid residues (85%) identical to, and 1682 of 1846 amino acid residues (91%) similar to, the 1943 amino acid residue ptnr:TREMBLNEW-ACC:AAG53891 protein from Mus musculus (Mouse) (PROTOCADHERIN). Public amino acid databases include the GenBank databases, SwissProt, PDB and PIR.

NOV8a is expressed in at least brain, lymphoid tissue, placenta. This information was derived by determining the tissue sources of the sequences that were included in the invention including but not limited to SeqCalling sources, Public EST sources, Literature sources, and/or RACE sources.

NOV8b

A disclosed NOV8b nucleic acid of 6003 nucleotides (also referred to as CG57452-02) encoding a protocadherin-like protein is shown in Table 8C. Putative untranslated regions upstream and/or downstream from the coding region, if any, are underlined, and the start and stop codons are in bold letters.

TABLE 8C
NOV8b nucleotide sequence.

| | |
|---|---|
| CAAATGTTTCTTTCTACCTTTTCTTATTTCAGATCAGCTTTGAGTGAACTTTGACAGAAG | (SEQ ID NO:19) |
| ATGTTTCGACAGTTTTATCTCTGGACATGTTTAGCTTCAGGGATCATCCTGGGCTCTCTC | |
| TTTGAAATCTGCTTGGGCCAGTATGATGATGGTAAGGATTGCAAACTAGCTAGGGGAGGA | |
| CCACCAGCTACCATAGTTGCTATTGATGAAGAAAGTCGGAATGGTGCAGGTACAATTCTG | |
| GTGGACAACATGCTGATCAAAGGGACTGCTGGAGGACCAGACCCCACCATAGAACTTTCT | |
| TTAAAGGATAATGTGGATTACTGGGTGTTGATGGATCCTGTTAAGCAAATGCTTTTCCTG | |
| AACAGCACCGGAAGAGTTCTGGATAGAGATCCACCGATGAACATACACTCCATTGTGGTG | |
| CAGGTCCAGTGCATCAACAAAAAAGTGGGCACTATTATCTACCATGAAGTGCGAATAGTG | |
| GTGAGAGACAGGAATGACAACTCACCCACTTTCAAGCATGAAAGCTACTATGCCACAGTG | |
| AATGAGCTCACTCCAGTTGGTACCACAATATTCACAGGATTTTCAGGAGACAATGGAGCT | |
| ACAGATATAGATGATGGACCAAATGGACAGATAGAGTATGTTATTCAGTATAATCCAGAT | |
| GATCCGACATCCAATGACACCTTTGAAATTCCCCTAATGTTGACTGGAAATATAGTGTTA | |
| AGGAAGAGGCTCAACTATGAAGATAAGACTCGCTACTTTGTCATAATCCAAGCTAATGAC | |
| CGTGCCCAAAATCTGAATGAGAGGCGAACCACCACCACCACTCTCACAGTGGATGTTCTG | |
| GATCGAGATGACTTGGGTCCAATGTTTCTTCCTTGTGTCCTTGTGCCAAACACTCGTGAT | |
| TGCCGTCCACTCACTTATCAAGCTGCCATACCTGAGTTGAGAACTCCGGAAGAACTGAAC | |
| CCCATTATTGTTACGCCACCAATCCAAGCCATTGATCAGGACCGGAATATTCAACCGCCA | |
| TCAGATAGGCCAGGAATCCTCTATTCCATCCTTGTTGGGACTCCTGAGGATTACCCACGA | |
| TTTTTCCATATGCATCCTAGGACAGCAGAACTTAGTCTCCTGGAGCCAGTAAACAGAGAC | |
| TTTCACCAGAAATTTGATTTGGTTATTAAGGCTGAACAAGACAATGGTCATCCTCTTCCT | |
| GCCTTTGCCGGTCTACACATTGAAATACTGGATGAAAACAATCAAAGTCCATATTTTACA | |
| ATGCCCAGTTATCAAGGCTATATCCTGGAATCTGCCCCAGTGGGAGCAACCATTTCGGAC | |
| AGTCTCAATTTGACCTCACCTTTAAGAATAGTAGCTCTGGACAAGGACATAGAAGATACA | |
| AAAGACCCAGAGCTTCACCTTTTTCTGAATGACTACACCTCAGTCTTCACCGTCACACAG | |
| ACTGGTATTACTCGCTACCTCACCTTACTTCAACCAGTGGACAGGGAAGAACAGCAAACT | |
| TACACCTTTTCGATAACAGCATTTGATGGTGTACAAGAAAGTGAGCCAGTCATCGTCAAT | |
| ATTCAAGTGATGGATGCAAATGATAACACGCCAACCTTCCCTGAAATATCCTATGATGTG | |
| TATGTTTATACAGACATGAGACCTGGGGACAGTGTCATACAGCTCACTGCAGTCGACGCA | |
| GACGAAGGGTCAAATGGGGAGATCACATATGAAATCCTTGTTGGGGCTCAGGGAGACTTC | |
| ATCATCAATAAAACAACAGGGCTTATCACCATCGCTCCAGGGGTGGAAATGATAGTCGGG | |
| CGGACTTACGCACTCACGGTCCAAGCAGCGGATAATGCTCCTCCTGCAGAGCGAAGGAAC | |
| TCCATCTGCACTGTGTATATTGAAGTGCTTCCACCAAATAATCAAAGCCCTCCTCGCTTC | |
| CCACAGCTGATGTATAGCCTTGAAATTAGTGAAGCCATGAGGGTTGGTGCTGTTTTATTA | |
| AATCTACAGGCAACTGATCGAGAGGGAGACTCAATAACATATGCCATTGAGAATGGAGAT | |
| CCTCAGAGAGTTTTTAATCTTTCAGAAACCACGGGGATTCTAACCTTAGGGAAAGCACTG | |
| GACAGGGAAAGCACTGATCGCTACATTCTGATCATCACAGCTTCAGATGGCAGGCCAGAT | |
| GGGACCTCAACTGCCACAGTAAACATAATGGTGACAGATGTCAATGACAATGCTCCAGTG | |
| TTTGATCCTTATCTGCCAAGAAATTTATCTGTGGTGGAAGAAGAAGCCAATGCCTTTGTG | |
| GGTCAAGTAAAAGCAACAGACCCTGATGCTGGAATAAATGGTCAAGTGCACTACAGTTTG | |
| GGTAACTTTAATAATCTTTTTCGTATCACATCCAATGGGAGCATTTACACAGCAGTGAAG | |
| CTTAACAGAGAAGTCAGGGACTACTATGAACTTGTTGTTGTGGCAACAGATGGAGCAGTA | |
| CACCCTCGTCATTCAACTCTAACCTTGGCCATCAAGGTTTTGGACATTGATGATAACAGT | |
| CCTGTGTTCACCAATTCAACATACACTGTCCTTGTTGAAGAGAATTTGCCAGCTGGGACT | |
| ACCATCCTTCAAATAGAGGCCAAAGATGTCGACCTTGGAGCAAATGTGTCTTACCGGATA | |
| AGAAGCCCAGAAGTGAAGCACTTTTTTGCACTACATCCATTTACAGGAGAACTATCGCTT | |
| TTAAGGAGTTTAGATTATGAGGCATTTCCAGACCAAGAAGCAAGTATCACTTTTCTGGTA | |
| GAGGCCTTTGATATTTATGGAACAATGCCACCTGGTATTGCTACTGTCACAGTGATTGTA | |
| AAGGATATGAATGATTATCCTCCTGTCTTTAGTAAACGAATATACAAAGGGATGGTGGCT | |
| CCGGATGCAGTCAAGGGTACACCTATCACAACAGTTTATGCTGAAGATGCAGACCCTCCT | |
| GGATTACCTGCAAGTCGTGTGAGGTATAGAGTAGATGATGTACAGTTTCCTTACCCTGCC | |
| AGTATTTTTGAAGTGGAAGAAGATTCTGGAAGAGTAATAACACGAGTCAATCTTAATGAA | |
| GAACCTACAACAATTTTTAAGTTGGTGGTGGTTGCTTTTGATGATGGGGAGCCTGTGATG | |
| TCCAGCAGTGCCACAGTGAAGATTCTTGTCTTACATCCTGGTGAGATCCCACGCTTCACA | |
| CAGGAGGAATATAGGCCTCCTCCAGTAAGTGAACTTGCCACCAAAGGGACCATGGTTGGT | |
| GTAATTTCTGCTGCTGCCATTAATCAAAGTATTGTGTACTCCATTCTTTCAGGAAATGAA | |
| GAAGATACATTTGGAATTAATAACATCACAGGTGTTATCTATGTGAATGGACCTCTGGAT | |
| TATGAGACCACGACAAGCTATGTACTTCGAGTCCAAGCTGATTCCCTGGAAGTGGTCCTT | |
| GCCAATCTCCGAGTTCCTTCAAAAAGTAATACAGCTAAAGTATACATTGAGATTCAGGAT | |
| GAAAATAATCATCCCCCAGTGTTTCAGAAAAAATTCTACATCGGAGGTGTATCTGAAGAT | |
| GCAAGAATGTTTACTTCTGTACTCAGAGTGAAGGCTACTGATAAAGATACTCCCAATTAT | |
| AGTGTCATGGCCTACAGACTCATAATACCACCAATTAAACAGGGAAAAGAAGGATTTGTA | |
| GTGGAAACATATACAGGGCTTATCAAAACTGCTATGCTCTTCCATAATATGAGGAGATCC | |
| TACTTCAAGTTTCAAGTTATTGCAACTGACGACTATGGGAAGGGACTGAGCGGCAAAGCC | |
| GATGTACTCGTAAGTGTCTCCGTGGTCAATCAGCTGGATATGCAAGTCATTGTTTCCAAT | |
| GTGCCTCCTACTCTAGTGGAAAAAAAGATAGAAGATCTTACAGAAATCTTGGATCGCTAT | |
| GTTCAGGAACAAATTCCTGGTGCCAAGGTCGTAGTGGAGTCCATTGGAGCTCGCCGGCAT | |
| GGAGATGCCTTTTCCCTAGAAGATTACACCAAATGTGACTTGACTGTCTATGCAATTGAC | |
| CCCCAAACCAACAGAGCCATCGATAGAAATGAGCTTTTTAAGTTTTTGGATGGCAAACTA | |
| CTTGATATCAATAAAGACTTTCAGCCGTATTATGGGGAAGGAGGACGCATTCTGGAGATC | |
| CGGACTCCAGAGGCAGTGACCAGCATTAAAAAGAGAGGAGAAAGTCTAGGATACACAGAA | |
| GGGGCCTTGTTGGCTCTGGCCTTCATCATCATCCTCTGCTGCATTCCTGCCATCTTGGTG | |
| GTTTTGGTCAGCTACAGACAGTTTAAAGTACGTCAAGCTGAGTGTACACGTCAAGCTGAG | |
| TGTACAAAGACTGCACGAATTCAGGCCGCATTACCCGCGGCTAAACCAGCAGTGCCGGCT | |
| CCTGCACCAGTGGCAGCGCCCCCGCCGCCGCCGCCGCCTCCGCCAGGTGCGCATCTCTAT | |
| GAAGAACTTGGAGACAGCTCAATGTCTTTTCTTTCAAGTCTTTTCCTTCTCTACCATTTT | |
| CAACAAAGCAGGGGAAATAACTCAGTCTCAGAAGACAGGAAACATCAACAAGTTGTGATG | |
| CCCTTTTCTTCCAATACTATTGAGGCTCACAAGTCAGCTCATGTAGACGGATCACTTAAG | |
| AGCAACAAACTGAAGTCTGCAAGAAAATTCACATTTCTATCTGATGAGGATGACTTAAGT | |
| GCCCATAATCCCCTTTATAAGGAAAACATAAGTCAAGTATCAACAAATTCAGACATTTCA | |
| CAGAGAACAGATTTTGTAGACCCATTTTCACCCAAAATACAAGCCAAGAGTAAGTCTCTG | |
| AGGGGCCCAAGAGAAAAGATTCAGAGGCTGTGGAGTCAGTCAGTCAGCTTACCCAGGAGG | |
| CTGATGAGGAAAGTTCCAAATAGACCAGAGATCATAGATCTGCAGCAGTGGCAAGGCACC | |
| AGGCAGAAAGCTGAAAATGAAAACACTGGAATCTGTACAAACAAAAGAGGTAGCAGCAAT | |
| CCATTGCTTACAACTGAAGAGGCAAATTTGACAGAGAAAGAGGAAATAAGGCAAGGTGAA | |
| ACACTGATGATAGAAGGAACAGAACAGTTGAAATCTCTCTCTTCAGACTCTTCATTTTGC | |
| TTTCCCAGGCCTCACTTCTCATTCTCCACTTTGCCAACTGTTTCAAGAACTGTGGAACTC | |
| AAATCAGAACCTAATGTCATCAGTTCTCCTGCTGAGTGTTCCTTGGAACTTTCTCCTTCA | |
| AGGCCTTGTGTTTTACATTCTTCACTCTCTAGGAGAGAGACACCTATTTGTATGTTACCT | |
| ATTGAAACCGAAAGAAATATTTTTGAAAATTTTGCCCATCCACCAAACATCTCTCCTTCT | |
| GCCTGTCCCCTTCCCCCTCCTCCTCCTATTTCTCCTCCTTCTCCTCCTCCTGCTCCTGCT | |
| CCTCTTGCTCCTCCTCCTGACATTTCTCCTTTTTCTCTTTTTTGTCCTCCTCCCTCTCCT | |
| CCTTCTATCCCTCTTCCTCTTCCTCCTCCTACATTTTTTCCACTTTCCGTTTCAACGTCT | |
| GGTCCCCCAACACCACCTCTTCTACCTCCATTTCCAACTCCTCTTCCTCCACCACCTCCT | |
| TCTATTCCTTGCCCTCCACCTCCTTCAGCTTCATTTCTGTCCACAGAGTGTGTCTGTATA | |
| ACAGGTGTTAAATGCACGACCAACTTGATGCCTGCCGAGAAAATTAAGTCCTCTATGACA | |
| CAGCTATCAACAACGACAGTGTGTAAAACAGACCCTCAGACAGAACCAAAAGGCATCCTC | |
| AGACACGTTAAAAACTTAGCAGAACTTGAAAAATCAGTAGCTAACATGTACAGTCAAATA | |
| GAAAAAAACTATCTACGCACAAATGTTTCAGAACTTCAAACTATGTGCCCTTCAGAAGTA | |
| ACAAATATGGAAATCACATCTGAACAAAACAAGGGGAGTTTGAACAATATTGTCGAGGGA | |
| ACTGAAAAACAATCTCACAGTCAATCTACTTCACTGTAATGTTGCTTTTCTTATTTTAGT | |
| CGG | |

In a search of public sequence databases, the NOV8b nucleic acid sequence has 3708 of 4369 bases (84%) identical to a gb:GENBANK-ID:AF281899|acc:AF281899.1 mRNA from Mus musculus (Mus musculus protocadherin (av) mRNA, complete cds). Public nucleotide databases include all GenBank databases and the GeneSeq patent database.

The disclosed NOV8b polypeptide (SEQ ID NO:20) encoded by SEQ ID NO:19 has 1972 amino acid residues and is presented in Table 8D using the one-letter amino acid code. Signal P, Psort and/or Hydropathy results predict that NOV8b has a signal peptide and is likely to be localized at the plasma membrane with a certainty of 0.6760. The most likely cleavage site is between amino acids 26 and 27.

TABLE 8D
Encoded NOV8b protein sequence.

| | |
|---|---|
| MFRQFYLWTCLASGIILGSLFEICLGQYDDGKDCKLARGGPPATIVAIDEESRNGAGTIL | (SEQ ID NO:20) |
| VDNMLIKGTAGGPDPTIELSLKDNVDYWVLMDPVKQMLFLNSTGRVLDRDPPMNIHSIVV | |
| QVQCINKKVGTIIYHEVRIVVRDRNDNSPTFKHESYYATVNELTPVGTTIFTGFSGDNGA | |
| TDIDDGPNGQIEYVIQYNPDDPTSNDTFEIPLMLTGNIVLRKRLNYEDKTRYFVIIQAND | |
| RAQNLNERRTTTTTLTVDVLDGDDLGPMFLPCVLVPNTRDCRPLTYQAAIPELRTPEELN | |
| PIIVTPPIQAIDQDRNIQPPSDRPGILYSILVGTPEDYPRFFHMHPRTAELSLLEPVNRD | |
| FHQKFDLVIKAEQDNGHPLPAFASLHIEILDENNQSPYFTMPSYQGYILESAPVGATISD | |
| SLNLTSPLRIVALDKDIEDTKDPELHLFLNDYTSVFTVTQTGITRYLTLLQPVDREEQQT | |
| YTFSITAFDGVQESEPVIVNIQVMDANDNTPTFPEISYDVYVYTDMRPGDSVIQLTAVDA | |
| DEGSNGEITYEILVGAQGDFIINKTTGLITIAPGVEMIVGRTYALTVQAADNAPPAERRN | |
| SICTVYIEVLPPNNQSPPRFPQLMYSLEISEAMRVGAVLLNLQATDREGDSITYAIENGD | |
| PQRVFNLSETTGILTLGKALDRESTDRYILIITASDGRPDGTSTATVNIVVTDVNDNAPV | |
| FDPYLPRNLSVVEEEANAFVGQVKATDPDAGINGQVHYSLGNFNNLFRITSNGSIYTAVK | |
| LNREVRDYYELVVVATDGAVHPRHSTLTLAIKVLDIDDNSPVFTNSTYTVLVEENLPAGT | |
| TILQIEAKDVDLGANVSYRIRSPEVKHFFALHPFTGELSLLRSLDYEAFPDQEASITFLV | |
| EAFDIYGTMPPGIATVTVIVKDMNDYPPVFSKRIYKGMVAPDAVKGTPITTVYAEDADPP | |
| GLPASRVRYRVDDVQFPYPASIFEVEEDSGRVITRVNLNEEPTTIFKLVVVAFDDGEPVM | |
| SSSATVKILVLHPGEIPRFTQEEYRPPPVSELATKGTMVGVISAAAINQSIVYSIVSGNE | |
| EDTFGINNITGVIYVNGPLDYETRTSYVLRVQADSLEVVLANLRVPSKSNTAKVYIEIQD | |
| ENNHPPVFQKKFYIGGVSEDARMFTSVLRVKATDKDTGNYSVMAYRLIIPPIKEGKEGFV | |
| VETYTGLIKTAMLFHNMRRSYFKFQVIATDDYGKGLSGKADVLVSVSVVNQLDMQVIVSN | |
| VPPTLVEKKIEDLTEILDRYVQEQIPGAKVVVESIGARRHGDAFSLEDYTKCDLTVYAID | |
| PQTNRAIDRNELFKFLDGKLLDINKDFQPYYGEGGRILEIRTPEAVTSIKKRGESLGYTE | |
| GALLALAFIIILCCIPAILVVLVSYRQFKVRQAECTRQAECTKTARIQAALPAAKPAVPA | |
| PAPVAAPPPPPPPPPGAHLYEELGDSSMSFLSSLFLLYHFQQSRGNNSVSEDRKHQQVVM | |
| PFSSNTIEAHKSAHVDGSLKSNKLKSARKFTFLSDEDDLSAHNPLYKENISQVSTNSDIS | |
| QRTDFVDPFSPKIQAKSKSLRGPREKIQRLWSQSVSLPRRLMRKVPNRPEIIDLQQWQGT | |
| RQKAENENTGICTNKRGSSNPLLTTEEANLTEKEEIRQGETLMIEGTEQLKSLSSDSSFC | |
| FPRPHFSFSTLPTVSRTVELKSEPNVISSPAECSLELSPSRPCVLHSSLSRRETPICMLP | |
| IETERNIFENFAHPPNISPSACPLPPPPPISPPSPPPAPAPLAPPPDISPFSLFCPPPSP | |
| PSIPLPLPPPTFFPLSVSTSGPPTPPLLPPFPTPLPPPPPSIPCPPPPSASFLSTECVCI | |
| TGVKCTTNLMPAEKIKSSMTQLSTTTVCKTDPQREPKGILRHVKNLAELEKSVANMYSQI | |
| EKNYLRTNVSELQTMCPSEVTNMEITSEQNKGSLNNIVEGTEKQSHSQSTSL | |

A search of sequence databases reveals that the NOV8b amino acid sequence has 3708 of 4369 bases (84%) identical to a gb:GENBANK-ID:AF281899|acc:AF281899.1 mRNA from Mus musculus (Mus musculus protocadherin (av) mRNA, complete cds). Public amino acid databases include the GenBank databases, SwissProt, PDB and PIR.

NOV8b is expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, Public EST sources, Literature sources, and/or RACE sources.

The disclosed NOV8a polypeptide has homology to the amino acid sequences shown in the BLASTP data listed in Table 8E.

TABLE 8E
BLAST results for NOV8a

| Gene Index/ | | Length | Identity | Positives | |
|---|---|---|---|---|---|
| Identifier | Protein/Organism | (aa) | (%) | (%) | Expect |
| gi\|16933555\|ref\|NP_ | protocadherin 15 | 1955 | 1954/1973 | 1955/1973 | 0.0 |
| 149045.2\| | precursor; Usher | | (99%) | (99%) | |
| (NM_033056) | syndrome 1F | | | | |
| | (autosomal | | | | |
| | recessive, | | | | |
| | severe) [Homo | | | | |
| | sapiens] | | | | |
| gi\|14581464\|gb\|AAK31804.1\| | protocadherin 15 | 1955 | 1955/1973 | 1955/1973 | 0.0 |
| (AY029237) | [Homo sapiens] | | (98%) | (98%) | |
| gi\|15072441\|gb\|AAK31581.1\| | protocadherin 15 | 1955 | 1949/1973 | 1955/1973 | 0.0 |
| (AY029205) | [Homo sapiens] | | (98%) | (98%) | |
| gi\|12963485\|ref\|NP_ | protocadherin 15; | 1943 | 1626/1977 | 1626/1977 | 0.0 |
| 075604.1\| | Ames waltzer [Mus | | (82%), | (82%), | |
| (NM_023115) | musculus] | | Positives = | Positives = | |
| | | | 1745/1977 | 1745/1977 | |
| | | | (88%) | (88%) | |
| gi\|18574084\|ref\|XP_ | protocadherin 15 | 936 | 924/928 | 925/928 | 0.0 |
| 053625.2\| | precursor [Homo | | (99%) | (99%) | |
| (XM_053625) | sapiens] | | | | |

Table 8F lists the domain descriptions from DOMAIN analysis results against NOV8. This indicates that the NOV8 sequence has properties similar to those of other proteins known to contain this domain.

TABLE 8F
Domain Analysis of NOV8

gnl|Smart|smart00112, CA, Cadherin repeats.; Cadherins are
glycoproteins involved in Ca2+ -mediated cell—cell adhesion. Cadherin
domains occur as repeats in the extracellular regions which are
thought to mediate cell—cell contact when bound to calcium.
CD-Length = 82 residues, 100.0% aligned
Score = 83.2 bits (204), Expect = 1e−16

The disclosed NOV8 polypeptides are members of the protocadherin family, which in turn is one of the six subfamilies of the cadherin superfamily. Cadherins are membrane-associated glycoproteins that mediate cell-cell interactions in a calcium-dependent fashion. Protocadherins may act as cell-cell recognition molecules and may be involved in signal transduction cascades.

The disclosed NOV8 polypeptides have homology to the mouse protocadherin whose mutant version causes the Ames waltzer mouse phenotype, which includes deafness and a balance disorder due to degeneration of the neuroepithelium of the inner ear. Mutant mice show abnormal stereocilia in the inner ear at a very early age. The gene of invention may therefore have a role in developmental processes, cellular communication and disease processes such as cancer.

The disclosed NOV8 nucleic acids of the invention encode a protocadherin-like protein includes the nucleic acid whose sequence is provided in Table 8A or 8C or a fragment thereof. The invention also includes a mutant or variant nucleic acid any of whose bases may be changed from the corresponding base shown in Table 8A or 8C while still encoding a protein that maintains its protocadherin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 42 percent of the bases may be so changed.

The disclosed NOV8 protein of the invention includes the protocadherin-like protein whose sequence is provided in Table 8B or 8D. The invention also includes a mutant or variant protein any of whose residues may be changed from the corresponding residue shown in Table 8B or 8D while still encoding a protein that maintains its protocadherin-like activities and physiological functions, or a functional fragment thereof. In the mutant or variant protein, up to about 15 percent of the residues may be so changed.

The invention further encompasses antibodies and antibody fragments, such as F_{ab} or (F_{ab})₂, that bind immunospecifically to any of the proteins of the invention.

The above defined information for this invention suggests that this protocadherin-like protein (NOV8) may function as a member of a “protocadherin family”. Therefore, the NOV8 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV8 nucleic acids and proteins of the invention are useful in potential therapeutic applications implicated in diseases including but not limited to various pathologies and disorders as indicated below. For example, a cDNA encoding the protocadherin-like protein (NOV8) may be useful in gene therapy, and the protocadherin-like protein (NOV8) may be useful when administered to a subject in need thereof. By way of nonlimiting example, the compositions of the present invention will have efficacy for treatment of patients suffering from Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegenerationhemophilia, hypercoagulation, idiopathic thrombocytopenic purpura, autoimmune disease, allergies, immunodeficiencies, transplantation, graft versus host disease (GVHD), lymphaedema, hearing loss, tinnitus, balance disorders, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, cancer, tissue degeneration, bacterial/viral/parasitic infection, or other pathologies or conditions. The NOV8 nucleic acid encoding the protocadherin-like protein of the invention, or fragments thereof, may further be useful in diagnostic applications, wherein the presence or amount of the nucleic acid or the protein are to be assessed.

NOV8 nucleic acids and polypeptides are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below. The disclosed NOV8 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV9

A disclosed NOV9 nucleic acid of 13700 nucleotides (also referred to as CG57625-01) encoding a protocadherin-like protein is shown in Table 9A. Putative untranslated regions upstream and/or downstream from the coding region, if any, are underlined, and the start and stop codons are in bold letters.

TABLE 9A
NOV9 nucleotide sequence.

| | |
|---|---|
| TCTCTCTTTCTTCCCTCCAGGATGGAAGTATGATGTGATGGATATAATTATGGGACACTG | (SEQ ID NO:21) |
| TGTGGGCACACGGCCTCCTGCTTGTTGCCTCATCCTCCTGCTTTTCAAGCTTTTGGCCAC | |
| TGTCTCCCAGGGGCTGCCAGGGACTGGACCCCTGGGCTTCCACTTCACACATTCCATTTA | |
| TAATGCTACCGTGTATGAGAACTCAGCAGCAACGACCTACGTCAACAGCCAGAGTAGAAT | |
| GGGCATCACCTTAATAGATCTATCCTGGGATATCAAATACAGAATAGTGTCCGGAGACGA | |
| GGAAGGCTTTTTCAAAGCAGAGGAAGTCATCATTGCAGATTTCTGTTTTCTCAGAATAAG | |
| AACTAAAGGTGGCAATTCTGCCATATTAAATAGGGAAATCCAGGATAATTATTTATTGAT | |
| AGTAAAAGGTTCTGTCAGAGGAGAGGATTTGGAAGCATGGACCAAAGTGAATATACAGGT | |
| TTTAGATATGAATGATCTGAGACCTTTGTTTTCACCCACAACATACTCTGTTACCATAGC | |
| AGAAAGCACACCTCTAAGGACTAGTGTTGCCCAGGTGACTGCAACAGACGCACATATTGG | |
| TTCCAATGGAGAATTCTACTACTACTTTAAAAATAAAGTTGATCTCTTTTCAGTTCACCC | |
| CACGAGTGGTGTCATCTCCTTAAGTGGTCGATTAAATTATGATGAAAAGAATAGGTATGA | |
| TCTGGAAATTTTGGCTGTGGACCGGGGAATGAAACTGTATGGGAACAATGGAGTGAGCAG | |
| TACTGCAAAGCTTTATGTTCACATTGAGCGCATAAATGAACATGCCCCAACAATCCATGT | |
| AGTCACTCATGTTCCTTTCTCGTTGGAAAAAGAGCCAACATATGCAGTGGTGACAGTTGA | |
| TGACTTAGATGATGGAGCGAATGGAGAGATCGAATCTGTTTCCATTGTGGCTGGGGATCC | |
| TTTAGATCAGTTCTTCCTGGCTAAGGAAGGAAAGTGGTTGAATGAGTACAAGATTAAGGA | |
| GAGGAAGCAGATTGACTGGGAGAGCTTTCCCTATGGCTACAATCTCACTCTTCAAGCAAA | |
| AGACAAGGGATCTCCTCAAAAATGTTCAGCATTAAAGGCAGTCTACATTGGCAACCCCAC | |
| AAGAGACACTGTCCCCATTAGATTTGAAAAAGAAGTGTACGATGTGAGCATAAGTGAATT | |
| TTCCCCTCCTGGTGTCGTGGTTGCTATAGTAAAATTAAGTCCTGAACCGATAGATGTGGA | |
| ATACAAATTATCTCCTGGTGAGGATGCAGTGTACTTTAAAATTAATCCTCGGTCGGGTCT | |
| GATTGTTACAGCACGGCCACTGAATACTGTTAAGAAGGAGGTTTATAAACTGGAGGTGAC | |
| AAACAAGGAAGGAGATTTAAAAGCACAGGTCACCATCAGCATAGAAGATGCAAATGACCA | |
| CACCCCAGAATTTCAGCAACCACTGTATGATGCTTATGTGAATGAAAGTGTCCCAGTGGG | |
| AACCAGCGTTCTAACAGTTTCAGCTTCTGATAAGGATAAAGGAGAAAATGGGTACATCAC | |
| CTATAGTATCGCTAGCCTGAATTTGTTACCATTTGTCATTAATCAGTTTACAGGTGTTAT | |
| TAGCACAACTGAAGAACTGGATTTTGAATCCTCCCCAGAAATTTACAGATTCATTGTTAG | |
| AGCCTCTGACTGGGGTTCACCATACCGCCATGAAAGTGAGGTCAATGTGACTATTCGAAT | |
| AGGAAATGTCAACGACAACAGCCCTCTCTTTGAAAAAGTGGCTTGCCAGGGAGTTATTTC | |
| ATATGACTTTCCAGTTGGTGGTCACATCACAGCAGTCTCAGCGATCGATATCGATGAACT | |
| TGAACTTGTAAAGTACAAAATCATTTCTGGAAATGAACTTGGCTTCTTTTATTTAAACCC | |
| AGATTCTGGTGTTTTACAGCTTAAAAAATCACTGACAAATTCTGGCATTAAAAATGGCAA | |
| TTTTGCCCTCAGAATTACAGCAACTGATGGAGAGAATCTTGCAGACCCCATGTCTATTAA | |
| CATTTCAGTCCTACATGGGAAAGTGTCTTCAAAGAGCTTCAGTTGCAGAGAAACTCGTGT | |
| GGCTCAAAAGCTGGCAGAGAAACTACTCATTAAGGCAAAAGCAAATGGGAAACTGAATCT | |
| GGAAGATGGATTTCTTGACTTTTATTCAATTAATAGACAGGGACCATATTTTGACAAGTC | |
| TTTTCCTTCTGATGTGGCTGTAAAGGAGGATCTGCCAGTTGGTGCTAACATTCTGAAGAT | |
| TAAAGCCTATGATGCCGACTCTGGCTTCAATGGAAAAGTGCTATTTACAATATCAGATGG | |
| AAATACGGATAGTTGCTTTAATATTGATATGGAGACTGGGCAGCTTAAAGTCCTTATGCC | |
| CATGGATCGAGAACACACAGACCTCTATCTCCTTAATATCACCATCTATGACTTAGGTAA | |
| TCCACAGAAATCGTCATGGAGACTGCTGACCATCAATGTGGAGGATGCTAATGACAATAG | |
| CCCAGTTTTTATTCAAGACAGTTACTCAGTTAACATTCTTGAAAGTTCAGGCATTGGTAC | |
| TGAAATCATTCAAGTGGAAGCCAGAGACAAAGACTTAGGTTCTAATGGTGAAGTGACTTA | |
| CTCAGTCTTGACAGATACACAGCAGTTTGCCATCAATAGCTCAACTGGAATCGTTTATGT | |
| AGCCGACCAGTTGGACCGGGAATCCAAAGCCAATTATTCTTTGAAAATAGAAGCCAGGGA | |
| CAAGGCAGAGAGTGGTCAGCAGCTGTTTTCAGTTGTCACTCTTAAAGTTTTTTTAGATGA | |
| TGTCAATGACTGCTCCCCAGCTTTCATTCCCAGTAGCTATAGTGTGAAGGTTCTTGAAGA | |
| TCTCCCTGTTGGCACTGTCATTGCTTGGCTTGAGACCCATGATCCAGATCTTGGACTGGG | |
| GGGTCAAGTGCGCTATTCTTTGGTCAATGACTATAATGGGAGATTTGAAATAGATAAAGC | |
| AAGTGGTGCCATCCGCTTGAGCAAAGAGCTTGATTATGAGAAACAGCAGTTCTATAACCT | |
| TACTGTGCGGGCCAAAGACAAAGGGCGGCCTGTCTCTCTGTCATCTGTTTCCTTTGTTGA | |
| GGTGGAAGTGGTGGATGTCAATGAAAACCTCCACACTCCCTATTTCCCAGACTTTGCTGT | |
| TGTTGGATCTGTAAAGGAAAACTCACGCATTGGAACAAGCGTGCTGCAGGTGACTGCTCG | |
| AGATGAAGACTCCGGAAGGGATGGAGAGATCCAGTACTCCATCAGGGATGGCAGTGGTCT | |
| TGGAAGGTTCAGTATAGACGACGAGAGTGGGGTCATCACTGCCGCAGACATTCTTGATCG | |
| GGAGACAATGGGGTCATACTGGCTAACAGTGTATGCCACAGACAGGGGCGTTGTTCCACT | |
| CTACTCCACCATTGAGGTCTACATTGAAGTTGAAGATGTGAATGACAATGCCCCGCTGAC | |
| CTCAGAACCCCTATATATTATCCTGGTCATGGATAAACATCCGAAGGACGTATCTGTCAT | |
| TCAGATCCAGGCTGAAGATCCTGACTCCAGTTCCAATGAAAAACTGACATACAGGATTAC | |
| AAGTGGAAATCCTCAGAATTTTTTGTGCATCAATATCAAAACAGGGCTGATTACAACAAC | |
| TTCAAGGAAATTGGATCGAGAACAGCAGGCAGAACATTTTCTGGAGGTGACTGTGACAGA | |
| TGGTGGTCCCTCTCCAAAACAGTCAACCATTTGGGTGGTGGTTCAGGTTCTAGATGAAAA | |
| TGACAACAAGCCCCAGTTCCCAGAGAAGGTCTACCAGATCAAGCTGCCAGAACGTGACCG | |
| AAAGAAGAGAGGAGAACCGATTTACAGGGCTTTTGCATTTGATAGAGATGAGGGCCCCAA | |
| CGCAGAAATCTCCTACAGTATTGTGGATGGGAATGATGACGGAAAGTTCTTTATTGACCC | |
| TAAAACTGGGATGGTTTCTTCTAGAAAGCAGTTTACAGCAGGCAGTTATGACATCCTAAC | |
| GATAAAGGCAGTGGACAATGGGCGCCCACAGAAATCCTCCACGGCCCGCCTCCACATTGA | |
| ATGGATTAAGAAACCACCCCCTTCACCTATACCATTGACCTTCGATGAGCCGTTTTATAA | |
| CTTCACAGTCATGGAAAGTGATAGAGTGACTGAAATTGTAGGGGTGGTGTCTGTGCAGCC | |
| AGCTAACACCCCTCTGTGGTTTGACATAGTTGGGGGGAATTTTGACAGCGCTTTTGATGC | |
| AGAGAAGGGTGTTGGGACAATTGTCATCGCAAAACCTTTGGATGCAGAGCAGAGGTCCAT | |
| CTATAATATGAGTGTGGAAGTCACCGATGGGACAAATGTTGCTGTTACTCAGGTATTTAT | |
| CAAAGTGCTGGATAATAATGATAATGGCCCAGAATTCTCTCAGCCGAATTACGATGTGAC | |
| AATTTCCGAGGATGTGCTTCCAGACACGGAGATCCTGCAGATTGAAGCCACAGATAGAGA | |
| TGAGAAGCACAAGCTGAGCTACACTGTTCATAGCAGCATCGACTCCATCAGCATGAGAAA | |
| ATTCCGGATTGACCCTAGCACTGGCGTGCTCTATACTGCCGAGAGGCTGGACCATGAGGC | |
| CCAGGACAAGCACATTCTCAACATAATGGTCAGAGATCAGGAGTTTCCTTATCGAAGAAA | |
| CTTGGCCCGAGTCATTGTGAATGTGGAGGATGCTAATGATCACAGTCCTTATTTTACCAA | |
| CCCACTGTATGAAGCGTCTGTGTTTGAATCTGCTGCTCTGGGATCAGCTGTTCTGCAAGT | |
| GACGGCTCTGGACAAAGACAAAGGAGAAAATGCAGAACTCATATATACCATAGAAGCAGG | |
| GAACACTGGGAACATGTTTAAGATCGAACCGGTCCTAGGCATCATCACCATTTGCAAAGA | |
| ACCAGACATGACGACGATGGGTCAGTTTGTCCTATCCATCAAAGTCACAGATCAGCGATC | |
| CCCGCCAATGTCTGCTACTGCAATTGTGCGCATTTCCGTCACCATGTCTGACAATTCTCA | |
| CCCCAAGTTCATTCACAAAGACTACCAAGCAGAAGTAAATCAAAATGTTGACATTGGAAC | |
| ATCAGTCATTCTAATCTCTGCCATCAGTCAATCTACCCTCATTTATGAAGTCAAAGATGG | |
| AGACATTAATGGGATCTTTACCATAAATCCATATTCTGGAGTCATCACCACTCAGAAGGC | |
| CCTGGATTATGAGCGCACATCCTCTTATCAACTCATCATTCAGGCCACCAATATGGCAGG | |
| AATGGCTTCCAATGCTACAGTCAATATTCAGATTGTTGATGAAAATGATAATGCCCCAGT | |
| TTTTCTCTTTTCTCAATACTCAGGCAGCCTAAGTGAGGCTGCCCCAATTAATAGCATTGT | |
| CAGGAGCTTGGATAACAGCCCACTGGTGATTCGAGCCACAGATGCTGACAGCAACCGGAA | |
| TGCTCTGCTTGTGTATCAGATTGTGGAGTCAACAGCAAAAAAGTTTTTCACGGTGGACTC | |
| CAGTACAGGTGCAATCAGAACAATTGCCAACCTGGACCATGAAACCATTGCCCATTTCCA | |
| TTTTCATGTGCATGTGAGAGACAGTGGTAGCCCCCAACTGACTGCAGAGAGTCCCGTTGA | |
| AGTCAACATTGAGGTGACAGATGTGAATGATAACCCACCTGTTTTTACTCAGGCTGTGTT | |
| TGAGACTATCTTACTTCTACCTACCTATGTTGGAGTGGAGGTTCTGAAAGTTAGTGCCAC | |
| AGATCCTGACTCTGAGGTACCCCCTGAACTGACATACAGCCTAATGGAAGGCAGTTTGGA | |
| TCATTTTTTAATTGACTCAAACAGTGGAGTACTTACCATAAAAAACAACAACCTCTCCAA | |
| GGATCACTACATGCTGATAGTTAAGGTGTCTGATGGAAAGTTCTACAGTACCTCCATGGT | |
| CACCATCATGGTTAAAGAAGCCATGGACAGCGGCCTCCACTTTACACAAAGCTTCTATTC | |
| CACCTCAATCTCAGAGAACAACACTAACATAACCAAAGTTGCTATTGTCAATGCAGTTGG | |
| AAATCGCCTTAATGAGCCCTTAAAATACAGCATCTTAAACCCACGAAATAAGTTCAAGAT | |
| AAAATCTACCTCAGGGGTCATTCAGACGACTGGAGTCCCCTTTGACCGTGAAGAACAAGA | |
| GTTATATGAGCTGGTGGTAGAAGCCAGCCGTGAGCTGGACCATCTGCGTGTGGCCAGAGT | |
| GGTGGTCAGGGTTAACATTGAAGACATAAATGACAATTCTCCAGTCTTTGTGGGCCTCCC | |
| ATACTATGCTGCTGTTCAAGTGGATGCGGAACCCGGGACTCTGATTTATCAGGTGACAGC | |
| CATTGACAAAGATAAAGGTCCAAATGGAGAAGTGACCTATGTCCTGCAGGATGACTATGG | |
| CCACTTTGAAATTAACCCTAATTCAGGGAATGTTATTTTAAAGGAAGCATTCAACTCTGA | |
| CTTGTCCAACATTGAGTATGGAGTCACCATCCTAGCCAAGGATGGCGGAAAACCTTCTTT | |
| GTCTACATCTGTGGAGCTTCCCATCACTATTGTCAACAAAGCAATGCCTGTGTTTGATAA | |
| GCCCTTTTATACAGCATCTGTCAATGAAGACATCAGAATGAACACACCCATCCTAAGCAT | |
| CAATGCCACCAGTCCAGAAGGCCAAGGCATCATATATATCATTATCGATGGGGACCCTTT | |
| TAAACAGTTTAACATTGACTTTGACACTGGGGTCCTGAAAGTTGTTAGCCCTTTGGATTA | |
| TGAAGTTACATCTGCTTACAAGCTGACAATAAGAGCCAGCGACGCCCTTACTGGTGCTAG | |
| GGCTGAAGTCACTGTTGACTTGCTAGTTAATGATGTAAATGACAACCCCCCTATTTTCGA | |
| TCAGCCTACATACAATACAACACTATCAGAAGCATCTCTTATTGGGACACCTGTTTTACA | |
| AGTTGTCTCTATTGATGCAGACTCAGAAAACAATAAAATGGTACATTATCAGATTGTCCA | |
| GGATACCTACAATAGCACAGATTATTTTCACATAGATAGCTCAAGTGGCTTAATCCTGAC | |
| AGCACGAATGCTGGACCATGAGTTAGTACAACACTGCACTTTGAAAGTCAGATCAATAGA | |
| TAGTGGCTTCCCATCACTGAGCAGTGAGGTTCTCGTTCATATCTACATCTCTGATGTAAA | |
| TGACAACCCTCCAGTTTTTAATCAGCTCATTTATGAGTCATATGTGAGTGAATTAGCCCC | |
| CCGGGGCCATTTTGTAACCTGTGTACAAGCCTCTGATGCAGACAGCTCTGATTTTGACCG | |
| GTTGGAATATAGCATTTTATCTGGGAATGACCGGACGAGCTTTCTGATGGACAGCAAGAG | |
| TGGAGTTATCACATTGTCCAACCATCGGAAGCAGCGGATGGAGCCTCTGTACAGTCTCAA | |
| TGTGTCTGTCTCTGATGGGTTGTTCACCAGCACTGCACAGGTGCATATTAGGGTACTTGG | |
| GGCTAACTTGTACAGCCCTGCCTTTTCACAAAGCACATACGTAGCTGAGGTGAGAGAGAA | |
| CGTGGCTGCAGGAACAAAGGTAATTCATGTTCGAGCCACAGATGGTGATCCAGGGACTTA | |
| TGGGCAGATCAGCTATGCCATCATCAATGACTTTGCCAAGGATCGATTCCTCATAGACAG | |
| CAATGGGCAGGTCATCACCACAGAAAGGCTAGACCGGGAAAACCCTCTAGAAGGGGATGT | |
| TAGTATTTTTGTGAGGGCCCTTGATGGTGGAGGGAGAACAACTTTCTGCACTGTGAGAGT | |
| GATTGTTGTGGATGAAAATGACAATGCTCCCCAGTTCATGACAGTGGAATATAGAGCCAG | |
| TGTCAGGGCAGATGTTGGAAGGGGCCACTTGGTCACTCAAGTTCAAGCCATAGATCCCGA | |
| TGATGGACCAAATTCAAGGATTACTTATTCCCTCTATAGCGAGGCCTCTGTTTCAGTGGC | |
| CGACCTCCTGGAAATCGATCCTGACAATGGCTGGATGGTCACAAAGGGTAATTTTAACCA | |
| GCTGAAAAATACAGTGCTTTCGTTCTTTGTCAAAGCAGTAGATGGGGGCATCCCAGTAAA | |
| GCACTCCCTCATTCCTGTCTATATCCACGTCTTGCCCCCTGAAACGTTCTTGCCATCATT | |
| CACCCAGTCTCAGTATTCCTTTACCATTGCAGAAGATACAGCCATTGGGAGTACAGTGGA | |
| CACCCTGAGGATTTTGCCCAGTCAGAATGTCTGGTTCAGCACAGTTAATGGGGAACGGCC | |
| AGAAAATAACAAAGGGGGCATATTCGTCATAGAACAGGAAACAGGCACTATTAAGCTTGA | |
| CAAACGCCTTGACCGTGAAACCAGCCCAGCTTTCCACTTTAAAGTAGCAGCCACTATACC | |
| CCTGGACAAAGTAGACATTGTGTTTACTGTGGATGTAGATATCAAGGTATTGGATTTGAA | |
| TGACAACAAGCCAGTCTTTGAAACTTCAAGCTATGACACCATTATAATGGAAGGGATGCC | |
| TGTTGGCACCAAACTCACACAAGTGAGAGCTATTGATATCGACTGGGGAGCCAATGGACA | |
| AGTCACTTACTCCCTCCACTCGGATTCCCAGCCCGAAAAGGTAATGGAAGCATTCAATAT | |
| TGACAGCAACACGGGCTGGATCAGTACCTTGAAGGACCTAGATCACGAGACAGACCCCAC | |
| ATTCACCTTCTCTGTGGTGGCCTCTGACCTTGGAGAGGCATTCTCTCTTTCCTCCACGGC | |
| CTTGGTCTCTGTCAGAGTGACAGATATAAATGACAATGCACCAGTCTTCGCGCAGGAAGT | |
| GTACCGAGGGAATGTGAAGGAGAGCGACCCACCGGGCGAGGTGGTAGCCGTCCTCAGCAC | |
| CTGGGACAGAGACACATCCGACGTTAATCGCCAAGTGAGCTACCATATTACAGGTGGAAA | |
| CCCTCGAGGAAGGTTTGCTCTGGGCCTGGTGCAAAGTGAGTGGAAGGTCTATGTGAAGAG | |
| GCCTCTAGACAGAGAAGAACAGGACATTTACTTTCTCAATATCACTGCCACTGATGGGCT | |
| TTTTGTCACACAGGCCATGGTGGAAGTGAGCGTCAGTGATGTGAATGACAATAGCCCAGT | |
| GTGTGATCAGGTTGCATATACAGCATTACTTCCTGAAGACATTCCATCAAATAAAATCAT | |
| CCTGAAAGTCAGTGCAAAGGATGCTGATATTGGATCCAATGGATATATACGATACTCACT | |
| CTATGGATCTGGAAACAGTGAATTTTTTCTAGATCCAGAAAGTGGCGAGTTAAAAACCTT | |
| GGCTCTGTTGGACCGGGAGAGGATCCCCGTGTACAGCCTGATGGCCAAGGCCACTGACGG | |
| GGGTGGCAGGTTCTGCCAGTCCAACATCCACCTAATCCTGGAGGATGTGAATGATAACCC | |
| CCCTGTGTTTTCTTCTGACCACTACAACACCTGTGTCTATGAGAACACAGCCACCAAGGC | |
| TCTGTTGACCAGAGTTCAAGCCGTGGACCCCGACATTGGCATCAATAGGAAGGTCGTGTA | |
| CTCCCTGGCAGACTCAGCTGGTGGGGTCTTCTCCATTGACAGCTCATCTGGCATCATCAT | |
| CCTGGAGCAGCCACTGGACCGTGAGCAGCAGTCTTCGTACAACATCAGCGTGCGGGCCAC | |
| TGACCAGAGTCCTGGACAGTCCCTGTCCTCTCTCACTACTGTCACCATCACCGTTCTGGA | |
| CATTAATGACAACCCCCCTGTGTTTGAGAGGAGGGACTACCTGGTGACGGTGCCTGAGGA | |
| CACCTCCCCTGGCACCCAAGTCCTTGCTGTTTTTGCCACCAGCAAAGATATTGGCACAAA | |
| TGCTGAGATCACTTATCTCATCCGGTCTGGGAACGAACAAGGGAAATTTAAGATCAACCC | |
| CAAGACAGGGGGTATTTCTGTCTCTGAAGTCCTGGACTATGAATTATGCAAAAGGTTTTA | |
| CCTGGTAGTGGAAGCCAAAGATGGGGGCACCCCAGCTCTCAGCGCTGTGGCCACTGTCAA | |
| CATCAACCTCACAGATGTTAATGACAACCCTCCCAAGTTCAGCCAAGACGTCTACAGTGC | |
| GGTTATCAGTGAAGACGCCTTGGTGGGAGACTCTGTCATTTTGCTAATAGCAGAAGATGT | |
| AGACAGCCAGCCCAACGGACAGATTCATTTTTCCATTGTGAATGGAGATCGGGACAATGA | |
| ATTTACTGTAGATCCTGTCTTGGGACTTGTGAAAGTTAAGAAGAAATTGGACCGGGAACG | |
| GGTGTCTGGATACTCTCTGCTTGTCCAGGCCGTAGACAGTGGCATTCCTGCAATGTCATC | |
| AACTGCAACTGTCAACATTGATATTTCTGATGTGAATGACAACAGCCCGGTGTTTACACC | |
| TGCCAACTATACTGCTGTGATTCAGGAAAATAAGCCAGTGGGCACCAGCATCTTGCAGCT | |
| GGTGGTGACAGACAGAGACTCCTTTCACAATGGGCCTCCCTTTTCATTCTCTATTTTGTC | |
| GGGAAATGAAGAGGAGGAGTTTGTGTTGGACCCTCATGGGATCTTGCGGTCGGCTGTGGT | |
| CTTCCAGCACACAGAGTCTCTGGAATACGTGTTGTGTGTCCAGGCAAAGGATTCAGGCAA | |
| ACCCCAGCAAGTTTCTCACACTTACATCCGCGTGCGAGTCATTGAGGAAAGCACCCACAA | |
| GCCCACAGCCATTCCCCTGGAAATTTTCATTGTCACCATGGAGGATGACTTTCCTGGTGG | |
| GGTCATTGGGAAGATTCATGCCACAGATCAAGACATGTATGATGTGCTCACATTTGCCCT | |
| GAAATCGGAGCAGAAAAGCTTATTTAAAGTGAACAGTCACGATGGGAAAATCATCGCCCT | |
| GGGAGGCCTGGACAGCGGCAAGTATGTCCTGAATGTGTCTGTGAGTGATGGTCGCTTCCA | |
| GGTACCCATTGATGTGGTCGTGCATGTGGAGCAGTTGGTGCATGAGATGCTGCAGAACAC | |
| TGTCACCATCCGCTTTGAAAATGTGTCCCCTGAGGACTTCGTGGGGCTGCACATGCATGG | |
| GTTCCGGCGCACCCTGCGGAATGCAGTCCTCACCCAGAAGCAGGACAGCCTGCGCATCAT | |
| CAGCATCCAGCCCGTGGCAGGCACCAACCAACTGGACATGCTGTTTGCGGTGGAGATGCA | |
| CAGCAGCGAGTTCTACAAGCCAGCCTACCTGATCCAGAAGCTGTCCAATGCTAGAAGACA | |
| CCTGGAGAATATCATGCGCATCTCAGCCATCTTGGAGAAGAACTGCTCAGGGCTGGACTG | |
| TCAGGAACAGCATTGTGAGCAAGGCTTGTCACTCGATTCCCACGCGCTCATGACCTACAG | |
| CACGGCTCGCATCAGCTTTGTGTGTCCGCGTTTCTACAGGAACGTGCGTTGCACCTGCAA | |
| TGGTGGACTGTGTCCGGGGTCCAACGATCCTTGTGTGGAGAAGCCGTGTCCAGGGGACAT | |
| GCAGTGTGTCAGTTATGAAGCCACCAGGAGACCGTTCCTCTGCCAGTCTCCACCAGGGAA | |
| GCTCGGAGAGTGCTCAGGGCACACTTCTCTCAGCTTTGCTGGAAACAGTTACATCAAATA | |
| TCGGCTTTCTGAAAATAGCAAAGAAGAGGATTTCAAACTAGCTCTGCGTCTTCGAACACT | |
| GCAAAGCAATGGGATTATAATGTACACCAGAGCAAATCCCTGCATAATTCTGAAGCAGAT | |
| TGTGGATCGCAAGCTGTGGTTCCAGCTGGACTGCGGCAGCGGCCCTGGAATCTTGGGCAT | |
| CTCGGGCCGTGCTGTCAACGACGGGAGCTGGCACTCGGTCTTCCTGGAGCTCAACCGCAA | |
| TTTCACGAGCCTGTCCCTGGATGACAGCTACGTGGAGCGCCGCCGGGCGCCCCTCTACTT | |
| CCAGACGCTGAGCACTGAGAGTAGCATCTACTTCGGCGCCCTGGTGCAAGCGGATAACAT | |
| CCGCAGCCTGACTGACACGCGGGTCACGCAGGTGCTCAGCGGCTTCCAGGGCTGCCTGGA | |
| CTCGGTGATACTGAATAACAATGAGCTCCCGCTGCAGAACAAGCGCAGCAGCTTCGCGGA | |
| GGTGGTGGGCCTGACGGAGCTGAAGCTGGGCTGCGTGCTCTATCCCGACGCCTGCAAGCG | |
| CAGCCCGTGCCAGCACGGGGGCAGCTGCACTGGCCTGCCATCGGGGGGTGGCTATCAGTG | |
| TACCTGTCTCTCACAGTTTACGGGGAGAAACTGTGAATCTGAGATTACAGCCTGCTTCCC | |
| AAACCCCTGCCGGAATGGAGGATCCTGCGATCCAATAGGAAACACTTTCATCTGCAATTG | |
| TAAAGCTGGGCTCACTGGAGTCACGTGTGAGGAGGACATCAATGAGTGCGAACGAGAGGA | |
| GTGTGAGAACGGAGGCTCCTGCGTGAACGTGTTCGGCTCCTTCCTCTGCAACTGCACGCC | |
| GGGCTACGTGGGCCAGTACTGCGGGCTGCGCCCCGTGGTGGTACCCAATATCCAGGCTGG | |
| CCACTCCTACGTGGGGAAGGAGGAGCTCATCGGCATCGCCGTGGTCCTCTTCGTCATCTT | |
| CATCCTGGTGGTTCTCTTCATAGTCTTCCGCAAGAAGGTCTTCCGCAAGAACTACTCCCG | |
| CAACAACATCACGCTAGTGCAGGACCCGCCCACCGCCGCCCTGCTTAACAAGAGCAATGG | |
| CATCCCGTTCCGGAACCTGCGCGGCAGTGGGGACGGCCGCAACGTCTACCAGGAGGTGGG | |
| GCCCCCGCAGGTCCCCGTGCGCCCCATGGCCTACACACCCTGCTTCCAGAGTGACTCCAG | |
| GAGCAACCTGGATAAGATCGTGGACGGGCTGGGAGGCGAGCACCAGGAAATGACCACGTT | |
| TCACCCTGAGTCGCCCCGCATCCTGACAGCCCGGCGGGGCGTGGTCGTGTGCAGTGTGGC | |
| CCCCAACCTCCCCGCCGTGTCACCCTGCCGCTCCGACTGCGACTCCATCCGGAAGAATGG | |
| CTGGGACGCGGGAACTGAGAGTAATAAAGGCAGCAACTCTGAAGTTCAGTCCCTCAGCTC | |
| CTTCCAGTCAGATTCTGGTGACGACAATGCCTATCACTGGGACACCTCTGATTGGATGCC | |
| AGGGGCCCGCCTGTCGGACATAGAGGAAGTGCCCAACTATGAGAACCAGGATGGAGGGTC | |
| TGCACACCAGGGGAGCACACGGGAGCTGGAGAGCGATTACTACCTGGGTGGTTATGACAT | |
| TGACAGTGAATACCCACCCCCTCATGAAGAGGAGTTCTTGAGTCAGGACCAGCTGCCTCC | |
| TCCTCTCCCGGAGGACTTCCCAGACCAATATGAGGCCCTGCCACCCTCCCAGCCTGTCTC | |
| CCTGGCCAGCACACTGAGCCCAGACTGCAGGAGAAGGCCCCAGTTTCATCCTAGCCAGTA | |
| TCTCCCTCCTCACCCATTCCCCAACGAAACGGATTTGGTGGGCCCGCCTGCCAGCTGTGA | |
| ATTTAGTACTTTTGCTGTGAGCATGAACCAGGGCACAGAGCCCACAGGCCCAGCAGACAG | |
| CGTGTCTCTGTCCTTGCACAATTCCAGAGGCACCTCATCCTCGGATGTGTCTGCCAACTG | |
| CGGCTTTGACGATTCCGAAGTAGCCATGAGTGACTACGAGAGCGTGGGAGAGCTCAGCCT | |
| CGCCAGCCTTCACATTCCCTTTGTGGAGACTCAGCATCAGACTCAAGTGTAGACATCACA | |
| TCTTGGGTACTTCACCCTGT | |

In a search of public sequence databases, the NOV9 nucleic acid sequence, located on chromsome 11 has 4976 of 7882 bases (63%) identical to a gb:GENBANK-ID:AF100960|acc:AF100960.1 mRNA from Rattus norvegicus (Rattus norvegicus protocadherin (Fat) mRNA, complete cds). Public nucleotide databases include all GenBank databases and the GeneSeq patent database.

The disclosed NOV9 polypeptide (SEQ ID NO:22) encoded by SEQ ID NO:21 has 4544 amino acid residues and is presented in Table 9B using the one-letter amino acid code. Signal P, Psort and/or Hydropathy results predict that NOV9 has 2 signal peptide and is likely to be localized extracellularly with a certainty of 0.4600. The most likely cleavage site is between residues 32 and 33.

TABLE 9B
Encoded NOV9 protein sequence.

| | |
|---|---|
| MDIIMGHCVGTRPPACCLILLLFKLLATVSQGLPGTGPLGFHFTHSIYNATVYENSAART | (SEQ ID NO:22) |
| YVNSQSRMGITLIDLSWDIKYRIVSGDEEGFFKAEEVIIADFCFLRIRTKGGNSAILNRE | |
| IQDNYLLIVKGSVRGEDLEAWTKVNIQVLDMNDLRPLFSPTTYSVTIAESTPLRTSVAQV | |
| TATDADIGSNGEFYYYFKNKVDLFSVHPTSGVISLSGRLNYDEKNRYDLEILAVDRGMKL | |
| YGNNGVSSTAKLYVHIERINEHAPTIHVVTHVPFSLEKEPTYAVVTVDDLDDGANGEIES | |
| VSIVAGDPLDQFFLAKEGKWLNEYKIKERKQIDWESFPYGYNLTLQAKDKGSPQKCSALK | |
| AVYIGNPTRDTVPIRFEKEVYDVSISEFSPPGVVVAIVKLSPEPIDVEYKLSPGEDAVYF | |
| KINPRSGLIVTARPLNTVKKEVYKLEVTNKEGDLKAQVTISIEDANDHTPEFQQPLYDAY | |
| VNESVPVGTSVLTVSASDKDKGENGYITYSIASLNLLPFVINQFTGVISTTEELDFESSP | |
| EIYRFIVRASDWGSPYRHESEVNVTIRIGNVNDNSPLFEKVACQGVISYDFPVGGHITAV | |
| SAIDIDELELVKYKIISGNELGFFYLNPDSGVLQLKKSLTNSGIKNGNFALRITATDGEN | |
| LADPMSINISVLHGKVSSKSFSCRETRVAQKLAEKLLIKAKANGKLNLEDGFLDFYSINR | |
| QGPYFDKSFPSDVAVKEDLPVGANILKIKAYDADSGFNGKVLFTISDGNTDSCFNIDMET | |
| GQLKVLMPMDREHTDLYLLNITIYDLGNPQKSSWRLLTINVEDANDNSPVFIQDSYSVNI | |
| LESSGIGTEIIQVEARDKDLGSNGEVTYSVLTDTQQFAINSSTGIVYVADQLDRESKANY | |
| SLKIEARDKAESGQQLFSVVTLKVFLDDVNDCSPAFIPSSYSVKVLEDLPVGTVIAWLET | |
| HDPDLGLGGQVRYSLVNDYNGRFEIDKASGAIRLSKELDYEKQQFYNLTVRAKDKGRPVS | |
| LSSVSFVEVEVVDVNENLHTPYFPDFAVVGSVKENSRIGTSVLQVTARDEDSGRDGEIQY | |
| SIRDGSGLGRFSIDDESGVITAADILDRETMGSYWLTVYATDRGVVPLYSTIEVYIEVED | |
| VNDNAPLTSEPLYIILVMDKHPKDVSVIQIQAEDPDSSSNEKLTYRITSGNPQNFLCINI | |
| KTGLITTTSRKLDREQQAEHFLEVTVTDGGPSPKQSTIWVVVQVLDENDNKPQFPEKVYQ | |
| IKLPERDRKKRGEPIYRAFAFDRDEGPNAEISYSIVDGNDDGKFFIDPKTGMVSSRKQFT | |
| AGSYDILTIKAVDNGRPQKSSTARLHIEWIKKPPPSPIPLTFDEPFYNFTVMESDRVTEI | |
| VGVVSVQPANTPLWFDIVGGNFDSAFDAEKGVGTIVIAKPLDAEQRSIYNMSVEVTDGTN | |
| VAVTQVFIKVLDNNDNGPEFSQPNYDVTISEDVLPDTEILQIEATDRDEKHKLSYTVHSS | |
| IDSISMRKFRIDPSTGVLYTAERLDHEAQDKHILNIMVRDQEFPYRRNLARVIVNVEDAN | |
| DHSPYFTNPLYEASVFESAALGSAVLQVTALDKDKGENAELIYTIEAGNTGNMFKIEPVL | |
| GIITICKEPDMTTMGQFVLSIKVTDQGSPPMSATAIVRISVTMSDNSHPKFIHKDYQAEV | |
| NENVDIGTSVILISAISQSTLIYEVKDGDINGIFTINPYSGVITTQKALDYERTSSYQLI | |
| IQATNMAGMASNATVNIQIVDENDNAPVFLFSQYSGSLSEAAPINSIVRSLDNSPLVIRA | |
| TDADSNRNALLVYQIVESTAKKFFTVDSSTGAIRTIANLDHETIAHFHFHVHVRDSGSPQ | |
| LTAESPVEVNIEVTDVNDNPPVFTQAVFETILLLPTYVGVEVLKVSATDPDSEVPPELTY | |
| SLMEGSLDHFLIDSNSGVLTIKNNNLSKDHYMLIVKVSDGKFYSTSMVTIMVKEAMDSGL | |
| HFTQSFYSTSISENNTNITKVAIVNAVGNRLNEPLKYSILNPGNKFKIKSTSGVIQTTGV | |
| PFDREEQELYELVVEASRELDHLRVARVVVRVNIEDINDNSPVFVGLPYYAAVQVDAEPG | |
| TLIYQVTAIDKDKGPNGEVTYVLQDDYGHFEINPNSGNVILKEAFNSDLSNIEYGVTILA | |
| KDGGKPSLSTSVELPITIVNKAMPVFDKPFYTASVNEDIRMNTPILSINATSPEGQGIIY | |
| IIIDGDPFKQFNIDFDTGVLKVVSPLDYEVTSAYKLTIRASDALTGARAEVTVDLLVNDV | |
| NDNPPIFDQPTYNTTLSEASLIGTPVLQVVSIDADSENNKMVHYQIVQDTYNSTDYFHID | |
| SSSGLILTARMLDHELVQHCTLKVRSIDSGFPSLSSEVLVHIYISDVNDNPPVFNQLIYE | |
| SYVSELAPRGHFVTCVQASDADSSDFDRLEYSILSGNDRTSFLMDSKSGVITLSNHRKQR | |
| MEPLYSLNVSVSDGLFTSTAQVHIRVLGANLYSPAFSQSTYVAEVRENVAAGTKVIHVRA | |
| TDGDPGTYGQISYAIINDFAKDRFLIDSNGQVITTERLDRENPLEGDVSIFVRALDGGGR | |
| TTFCTVRVIVVDENDNAPQFMTVEYRASVRADVGRGHLVTQVQAIDPDDGANSRITYSLY | |
| SEASVSVADLLEIDPDNGWMVTKGNFNQLKNTVLSFFVKAVDGGIPVKHSLIPVYIHVLP | |
| PETFLPSFTQSQYSFTIAEDTAIGSTVDTLRILPSQNVWFSTVNGERPENNKGGIFVIEQ | |
| ETGTIKLDKRLDRETSPAFHFKVAATIPLDKVDIVFTVDVDIKVLDLNDNKPVFETSSYD | |
| TIIMEGMPVGTKLTQVRAIDMDWGANGQVTYSLHSDSQPEKVMEAFNIDSNTGWISTLKD | |
| LDHETDPTFTFSVVASDLGEAFSLSSTALVSVRVTDINDNAPVFAQEVYRGNVKESDPPG | |
| EVVAVLSTWDRDTSDVNRQVSYHITGGNPRGRFALGLVQSEWKVYVKRPLDREEQDIYFL | |
| NITATDGLFVTQAMVEVSVSDVNDNSPVCDQVAYTALLPEDIPSNKIILKVSAKDADIGS | |
| NGYIRYSLYGSGNSEFFLDPESGELKTLALLDRERIPVYSLMAKATDGGGRFCQSNIHLI | |
| LEDVNDNPPVFSSDHYNTCVYENTATKALLTRVQAVDPDIGINRKVVYSLADSAGGVFSI | |
| DSSSGIIILEQPLDREQQSSYNISVRATDQSPGQSLSSLTTVTITVLDINDNPPVFERRD | |
| YLVTVPEDTSPGTQVLAVFATSKDIGTNAEITYLIRSGNEQGKFKINPKTGGISVSEVLD | |
| YELCKRFYLVVEAKDGGTPALSAVATVNINLTDVNDNPPKFSQDVYSAVISEDALVGDSV | |
| ILLIAEDVDSQPNGQIHFSIVNGDRDNEFTVDPVLGLVKVKKKLDRERVSGYSLLVQAVD | |
| SGIPAMSSTATVNIDISDVNDNSPVFTPANYTAVIQENKPVGTSILQLVVTDRDSFHNGP | |
| PFSFSILSGNEEEEFVLDPHGILRSAVVFQHTESLEYVLCVQAKDSGKPQQVSHTYIRVR | |
| VIEESTHKPTAIPLEIFIVTMEDDFPGGVIGKIHATDQDMYDVLTFALKSEQKSLFKVNS | |
| HDGKIIALGGLDSGKYVLNVSVSDGRFQVPIDVVVHVEQLVHEMLQNTVTIRFENVSPED | |
| FVGLHMHGFRRTLRNAVLTQKQDSLRIISIQPVAGTNQLDMLFAVEMHSSEFYKPAYLIQ | |
| KLSNARRHLENIMRISAILEKNCSGLDCQEQHCEQGLSLDSHALMTYSTARISFVCPRFY | |
| RNVRCTCNGGLCPGSNDPCVEKPCPGDMQCVSYEASRRPFLCQCPPGKLGECSGHTSLSF | |
| AGNSYIKYRLSENSKEEDFKLALRLRTLQSNGIIMYTRANPCIILKQIVDGKLWFQLDCG | |
| SGPGILGISGRAVNDGSWHSVFLELNRNFTSLSLDDSYVERRRAPLYFQTLSTESSIYFG | |
| ALVQADNIRSLTDTRVTQVLSGFQGCLDSVILNNNELPLQNKRSSFAEVVGLTELKLGCV | |
| LYPDACKRSPCQHGGSCTGLPSGGGYQCTCLSQFTGRNCESEITACFPNPCRNGGSCDPI | |
| GNTFICNCKAGLTGVTCEEDINECEREECENGGSCVNVFGSFLCNCTPGYVGQYCGLRPV | |
| VVPNIQAGHSYVGKEELIGIAVVLFVIFILVVLFIVFRKKVFRKNYSRNNITLVQDPATA | |
| ALLNKSNGIPFRNLRGSGDGRNVYQEVGPPQVPVRPMAYTPCFQSDSRSNLDKIVDGLGG | |
| EHQEMTTFHPESPRILTARRGVVVCSVAPNLPAVSPCRSDCDSIRKNGWDAGTESNKGSN | |
| SEVQSLSSFQSDSGDDNAYHWDTSDWMPGARLSDIEEVPNYENQDGGSAHQGSTRELESD | |
| YYLGGYDIDSEYPPPHEEEFLSQDQLPPPLPEDFPDQYEALPPSQPVSLASTLSPDCRRR | |
| PQFHPSQYLPPHPFPNETDLVGPPASCEFSTFAVSMNQGTEPTGPADSVSLSLHNSRGTS | |
| SSDVSANCGFDDSEVAMSDYESVGELSLASLHIPFVETQHQTQV | |

A search of sequence databases reveals that the NOV9 amino acid sequence has 2201 of 4118 amino acid residues (53%) identical to, and 2931 of 4118 amino acid residues (71%) similar to, the 4589 amino acid residue ptnr:SPTREMBL-ACC:Q9WU10 protein from Rattus norvegicus (Rat) (PROTOCADHERIN). Public amino acid databases include the GenBank databases, SwissProt, PDB and PIR.

NOV9 is expressed in at least breast, prostate, bone marrow, brain, liver, stomach, pituitary, cartilage. This information was derived by determining the tissue sources of the sequences that were included in the invention including but not limited to SeqCalling sources, Public EST sources, Literature sources, and/or RACE sources.

The disclosed NOV9 polypeptide has homology to the amino acid sequences shown in the BLASTP data listed in Table 9C.

TABLE 9C

| BLAST results for NOV9 | | | | | |
|---|---|---|---|---|---|
| Gene Index/ | | Length | Identity | Positives | |
| Identifier | Protein/Organism | (aa) | (%) | (%) | Expect |
| gi\|18578022\|ref\|XP_ | similar to FAT | 3446 | 3319/3386 | 3320/3386 | 0.0 |
| 061871.2\| | tumor suppressor | | (98%) | (98%) | |
| (XM_061871) | (Drosophila) | | | | |
| gi\|4885229\|ref\|NP_005236.1\| | FAT tumor | 4620 | 2381/4620 | 3178/4620 | 0.0 |
| (NM_005245) | suppressor | | (51%) | (68%) | |
| | precursor | | | | |
| gi\|13929168\|ref\|NP_ | FAT tumor suppressor | 4589 | 2368/4619 | 3170/4619 | 0.0 |
| 114007.1\| | (Drosophila) homolog | | (51%) | (68%) | |
| (NM_031819) | [Rattus norvegicus] | | | | |
| gi\|6688786\|emb\|CAB65271.1\| | mouse fat 1 cadherin | 4587 | 2367/4633 | 3167/4633 | 0.0 |
| (AJ250768) | [Mus musculus] | | (51%) | (68%) | |
| gi\|13787217\|ref\|NP_ | FAT tumor | 4349 | 1854/4434 | 2635/4434 | 0.0 |
| 001438.1\| | suppressor 2 | | (41%) | (58%) | |
| (NM_001447) | precursor [Homo | | | | |
| | sapiens] | | | | |

Table 9D lists the domain descriptions from DOMAIN analysis results against NOV9. This indicates that the NOV9 sequence has properties similar to those of other proteins known to contain this domain.

TABLE 9D
Domain Analysis of NOV9

gnl|Smart|smart00112, CA, Cadherin repeats.; Cadherins are
glycoproteins involved in Ca2+ -mediated cell—cell adhesion. Cadherin
domains occur as repeats in the extracellular regions which are
thought to mediate cell—cell contact when bound to calcium.
CD-Length = 82 residues, 100.0% aligned
Score = 97.4 bits (241), Expect = 2e−20

TABLE 9E
Domain Analysis of NOV9

gnl|Pfam|pfam 00028, cadherin, Cadherin domain
CD-Length 92 residues, 100.0% aligned
Score = 94.4 bits (233), Expect = 1e−19

NOV9 is a member of the protocadherin family, which in turn is one of the six subfamilies of the cadherin superfamily. Cadherins are membrane-associated glycoproteins that mediate cell-cell interactions in a calcium-dependent fashion. Protocadherins may act as cell-cell recognition molecules and may be involved in signal transduction cascades.

NOV9 has homology to the rat protocadherin that is most related to the Drosophila FAT gene. The Drosophila FAT gene shows the presence of multiple characteristic cadherin domains and is likely involved in cell guidance, cell repulsion and/or cell adhesion. Recessive lethal mutations in the fat locus of Drosophila cause hyperplastic, tumor-like overgrowth of larval imaginal discs, defects in differentiation and morphogenesis, and death during the pupal stage. This indicates that the fat gene has a tumor suppressor function (See Mahoney et al., Cell Nov. 29, 1991;67(5):853-68).

The disclosed NOV nucleic acid of the invention encoding a protocadherin-like protein includes the nucleic acid whose sequence is provided in Table 9A or a fragment thereof. The invention also includes a mutant or variant nucleic acid any of whose bases may be changed from the corresponding base shown in Table 9A while still encoding a protein that maintains its protocadherin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37 percent of the bases may be so changed.

The disclosed NOV9 protein of the invention includes the protocadherin-like protein whose sequence is provided in Table 9B. The invention also includes a mutant or variant protein any of whose residues may be changed from the corresponding residue shown in Table 9B while still encoding a protein that maintains its protocadherin-like activities and physiological functions, or a functional fragment thereof. In the mutant or variant protein, up to about 47 percent of the residues may be so changed.

The invention further encompasses antibodies and antibody fragments, such as F_{ab} or (F_{ab})₂, that bind immunospecifically to any of the proteins of the invention.

The above defined information for this invention suggests that this protocadherin-like protein (NOV9) may function as a member of a “protocadherin family”. Therefore, the NOV9 nucleic acids and proteins identified here may be useful in potential therapeutic applications implicated in (but not limited to) various pathologies and disorders as indicated below. T he potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV9 nucleic acids and proteins of the invention are useful in potential therapeutic applications implicated in diseases including but not limited to various pathologies and disorders as indicated below. For example, a cDNA encoding the protocadherin-like protein (NOV9) may be useful in gene therapy, and the protocadherin-like protein (NOV9) may be useful when administered to a subject in need thereof. By way of nonlimiting example, the compositions of the present invention will have efficacy for treatment of patients suffering from hemophilia, hypercoagulation, idiopathic thrombocytopenic purpura, autoimmune disease, allergies, immunodeficiencies, transplantation, graft versus host disease, endocrine dysfunctions, diabetes, obesity, growth and reproductive disorders, Von Hippel-Lindau (VHL) syndrome, cirrhosis, transplantation, hypercalceimia, ulcers, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cancer, tissue degeneration, bacterial/viral/parasitic infections, or other pathologies or conditions. The NOV9 nucleic acid encoding the protocadherin-like protein of the invention, or fragments thereof, may further be useful in diagnostic applications, wherein the presence or amount of the nucleic acid or the protein are to be assessed.

NOV9 nucleic acids and polypeptides are further useful in the generation of antibodies that bind immuno-specifically to the novel NOV9 substances for use in therapeutic or diagnostic methods. These antibodies may be generated according to methods known in the art, using prediction from hydrophobicity charts, as described in the “Anti-NOVX Antibodies” section below. The disclosed NOV9 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV10

A disclosed NOV10 nucleic acid of 1071 nucleotides (also referred to as CG57553-01) encoding a T01C1.3-like protein is shown in Table 10A. Putative untranslated regions upstream and/or downstream from the coding region, if any, are underlined, and the start and stop codons are in bold letters.

TABLE 10A
NOV10 nucleotide sequence.

| | |
|---|---|
| ATGCATCAGTTTACCATCGTTTCATTCCATCCATCCAGGAGACTACACAGAAACAGAGAG | (SEQ ID NO:23) |
| GACTATGTGGAAAGAAGTGCTGAGTTTGCAGATGGTTTGCTCTCAAAAGCTTTGAAAGAC | |
| ATTCAGTCTGGAGCACTCGACATAAATAAAGCAGGCATACTTTATGGCATACCTCAAAAA | |
| ACTTTACTTCTTCACTTAGAAGCCTTACCAGCAGGGAAGCCTGCATCTTTTAAAAACAAA | |
| ACTCGAGATTTCCATGATAGTTATTCATATAAGGACAGTAAAGAAACTTGTGCAGTGCTG | |
| CAAAAAGTAGCCTTGTGGGCAAGAGCTCAAGCAGAGCGCACAGAAAAAAGTAAACTCAAT | |
| CTACTTGAAACCTCAGAAATAAAATTCCCAACAGCTTCCACTTACCTCCATCAGCTAACT | |
| CTACAGAAAATGGTCACTCAGTTTAAAGAAAAAAATGAAAGCCTCCAATATGAAACTTCA | |
| AATCCTACTGTACAGTTAAAAATTCCTCAGCTACGAGTAAGTTCTGTCTCAAAATCACAA | |
| CCTGATGGTTCTGGTCTGTTGGATGTTATGTATCAAGTTTCCAAAACCTCTTCAGTCCTA | |
| GAAGGATCAGCTCTCCAAAAACTGAAAAATATACTCCCTAAACAGAACAAAATAGAATGT | |
| TCTGGGCCTGTAACTCACTCAAGTGTTGACTCTTACTTTCTACATGGGGACCTCTCTCCT | |
| TTGTGTCTTAATTCTAAAAATGGAACAGTTGATGGAACCTCTGAAAATACTGAAGATGGA | |
| TTAGATCGAAAAGACAGTAACCAGCCCAGGAAAAAACGTGGCCGCTATCGGCAATATGAT | |
| CATGAAATAATGGAAGAAGCTATTGCAATGGTAATGAGCGGAAAAATGAGTGTTTCCAAA | |
| GCACAAGGAATTTATGGGGTACCTCACAGCACTTTAGAATACAAGGTAAAAGAAAGATCT | |
| GGAACACTGAAGACTCCTCCGAAGAAGAAACTACGATTACCAGACACTGGGTTATATAAT | |
| ATGACAGATTCAGGGACTGGCAGCTGCAAAAACAGCAGCAAGCCTGTGTAG | |

In a search of public sequence databases, the NOV10 nucleic acid sequence, located on chromsome 4 has 165 of 267 bases (61%) identical to a gb:GENBANK-ID:BGDNA66KD|acc:X87727.1 mRNA from Borrelia garinii (B.garinii p66 gene for 66 kDa protein). Public nucleotide databases include all GenBank databases and the GeneSeq patent database.

The disclosed NOV10 polypeptide (SEQ ID NO:24) encoded by SEQ ID NO:23 has 356 amino acid residues and is presented in Table 10B using the one-letter amino acid code. Signal P, Psort and/or Hydropathy results predict that NOV10 has no signal peptide and is likely to be localized in the nucleus with a certainty of 0.7000.

TABLE 10B
Encoded NOV10 protein sequence.

| | |
|---|---|
| MHQFTIVSFHPSRRLHRNREDYVERSAEFADGLLSKALKDIQSGALDINKAGILYGIPQK | (SEQ ID NO:24) |
| TLLLHLEALPAGKPASFKNKTRDFHDSYSYKDSKETCAVLQKVALWARAQAERTEKSKLN | |
| LLETSEIKFPTASTYLHQLTLQKMVTQFKEKNESLQYETSNPTVQLKIPQLRVSSVSKSQ | |
| PDGSGLLDVMYQVSKTSSVLEGSALQKLKNILPKQNKIECSGPVTHSSVDSYFLHGDLSP | |
| LCLNSKNGTVDGTSENTEDGLDRKDSKQPRKKRGRYRQYDHEIMEEAIAMVMSGKMSVSK | |
| AQGIYGVPHSTLEYKVKERSGTLKTPPKKKLRLPDTGLYNMTDSGTGSCKNSSKPV | |

A search of sequence databases reveals that the NOV10 amino acid sequence has 47 of 144 amino acid residues (32%) identical to, and 77 of 144 amino acid residues (53%) similar to, the 185 amino acid residue ptnr:SPTREMBL-ACC:Q22051 protein from Caenorhabditis elegans (T01C1.3 PROTEIN). Public amino acid databases include the GenBank databases, SwissProt, PDB and PIR.

NOV10 is expressed in at least brain and kidney. This information was derived by determining the tissue sources of the sequences that were included in the invention including but not limited to SeqCalling sources, Public ES T sources, Literature sources, and/or RACE sources.

The disclosed NOV10 polypeptide has homology to the amino acid sequences shown in the BLASTP data listed in Table 10C.

TABLE 10C
BLAST results for NOV10

| Gene Index/ | | Length | Identity | Positives | |
|---|---|---|---|---|---|
| Identifier | Protein/Organism | (aa) | (%) | (%) | Expect |
| gi\|18559097\|ref\|XP_ | protein XP_087581 | 213 | 213/213 | 213/213 | e−110 |
| 087581.1\| | [Homo sapiens] | | (100%) | (100%) | |
| (XM_087581) | | | | | |
| gi\|16549953\|dbj\|BAB70892.1\| | unnamed protein | 213 | 212/213 | 213/213 | e−109 |
| (AK055258) | product [Homo | | (99%) | (100%) | |
| | sapiens] | | | | |
| gi\|14017807\|dbj\|BAB47424.1\| | KIAA1795 protein | 572 | 94/242 | 125/242 | 7e−28 |
| (AB058698) | [Homo sapiens] | | (38%) | (50%) | |
| gi\|14744761\|ref\|XP_ | KIAA1795 protein | 433 | 94/242 | 125/242 | 4e−26 |
| 050988.1\| | [Homo sapiens] | | (38%) | (50%) | |
| (XM_050988) | | | | | |
| gi\|18487927\|ref\|XP_ | Eip93F | 1165 | 37/76 | 46/76 | 6e−10 |
| 081696.1\| | [Drosophila | | (48%) | (59%) | |
| (XM_081696) | melanogaster] | | | | |

The T01C0.3-like protein described in this invention is similar to the C. elegans protein T01C1.3, a novel protein. The T01C1.3-like protein appears to be expressed in kidney and brain and may therefore play a role in the development of cancer, neurological diseases, or metabolic disorders. The T01C1.3-like gene maps to human chromosome 4 and has no identifiable domains.

The disclosed NOV10 nucleic acid of the invention encoding a T01C1.3-like protein includes the nucleic acid whose sequence is provided in Table 10A or a fragment thereof. The invention also includes a mutant or variant nucleic acid any of whose bases may be changed from the corresponding base shown in Table 10A while still encoding a protein that maintains its T01C1.3-like activities and physiological function is, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 39 percent of the bases may be so changed.

The disclosed NOV10 protein of the invention includes the T01C1.3-like protein whose sequence is provided in Table 10B. The invention also includes a mutant or variant protein any of whose residues may be changed from the corresponding residue shown in Table 10B while still encoding a protein that maintains its T01C0.3-like activities and physiological functions, or a functional fragment thereof. In the mutant or variant protein, up to about 68 percent of the residues may be so changed.

The invention further encompasses antibodies and antibody fragments, such as F_{ab} or (F_{ab})₂, that bind immunospecifically to any of the proteins of the invention.

The above defined information for this invention suggests that this T01C0.3-like protein (NOV10) may function as a member of a “T01C1.3 family”. Therefore, the NOV10 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV10 nucleic acids and proteins of the invention are useful in potential therapeutic applications implicated in diseases including but not limited to various pathologies and disorders as indicated below. For example, a cDNA encoding the T01C1.3-like protein (NOV10) may be useful in gene therapy, and the T01C0.3-like protein (NOV10) may be useful when administered to a subject in need thereof. By way of nonlimiting example, the compositions of the present invention will have efficacy for treatment of patients suffering from cancer, trauma, bacterial and viral infections, in vitro and in vivo regeneration, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, diabetes, autoimmune disease, renal artery stenosis, interstitial nephritis, glomerulonephritis, polycystic kidney disease, systemic lupus erythematosus, renal tubular acidosis, IgA nephropathy, and hypercalceimia, or other pathologies or conditions. The NOV10 nucleic acid encoding the T01C1.3-like protein of the invention, or fragments thereof, may further be useful in diagnostic applications, wherein the presence or amount of the nucleic acid or the protein are to be assessed.

NOV10 nucleic acids and polypeptides are further useful in the generation of antibodies that bind immuno-specifically to the novel NOV10 substances for use in therapeutic or diagnostic methods. These antibodies may be generated according to methods known in the art, using prediction from hydrophobicity charts, as described in the “Anti-NOVX Antibodies” section below. The disclosed NOV10 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV11

NOV11 includes three alpha-macroglobulin-like proteins disclosed below. The disclosed sequences have been named NOV11a, NOV11b and NOV11c.

NOV11a

A disclosed NOV11a nucleic acid of 6195 nucleotides (also referred to as CG57488_{—}01) encoding a alpha-macroglobulin-like protein is shown in Table 11A. Putative untranslated regions upstream and/or downstream from the coding region, if any, are underlined, and the start and stop codons are in bold letters.

TABLE 11A
NOV11a nucleotide sequence.

| | |
|---|---|
| ATGAGCGGCGCCCTGCTCTGGCCGTTGCTCCCGCTCCTGCTCCTGCTGCTGTCGGCGCGG | (SEQ ID NO:25) |
| GACGGCGTGCGCGCCGCGCAGCCTCAGGCCCCGGGTTACTTGATTGCAGCTCCCTCTGTT | |
| TTTCGCGCGGGCGTGGAGGAAGTCATCAGCGTGACCATCTTTAACTCTCCAAGGGAAGTC | |
| ACGGTCCAGGCTCAGCTGGTGGCCCAGGGTGAGCCGGTGGTGCAGAGCCAGGGAGCCATC | |
| CTGGATAAAGGGACAATCAAACTCAAGCATACGGTCCTCAGCACCTCCGGTATCTCCCTC | |
| CTGCCCATCCTTGCCCTGCTCTTGGGTGGCCGGGACCTTTCCTCCCTCTTCAGCCTCTGG | |
| CCAGTGTTGAGATATTTCCAGAAACAGGGCCAGGTGCCCACGGGCCTCCGGGGCCAAGCG | |
| CTTCTGAAAGTGTGGGGCCGCGGCTGGCAGGCGGAGGAGGGGCCCCTCTTTCACAACCAG | |
| ACCTCGGTGACCGTGGACGGCCGGGGCGCTTCTGTATTCATCCAGACGGACAAGCCTGTG | |
| TACAGACCCCAGCACCGAGTGCTCATAAGCATCTTCACCGTCTCTCCAAATCTGAGGCCT | |
| GTCAACGAGAAGCTGGAAGCCTACATCCTGGACCCCCGAGGCTCTCGGATGATAGAGTGG | |
| AGACACTTAAAGCCGTTCTGCTGCGGCATCACCAACATGAGCTTCCCCTTGTCCGACCAG | |
| CCTGTGTTGGGAGAATGGTTCATTTTTGTTGAAATGCAAGGCCACGCGTACAACAAGTCT | |
| TTTGAAGTTCAGAAGTATGTGTTGCCCAAGTTTGAGCTTCTGATTGACCCGCCCCGGTAT | |
| ATCCAAGACCTGGACGCCTGTGAGACAGGCACTGTGCGGGCCAGGTATACCTTTGGGAAA | |
| CCTGTGGCTGGTGCCTTAATGATCAACATGACTGTTAATGGTGTAGGGTACTACAGCCAC | |
| GAGGTGGGACGCCCTGTCCTCAGAACAACCAAGATCCTCGGCTCCCGGGACTTCGACATC | |
| TGCGTGAGGGACATGATCCCAGCCGACGTCCCTGAGCACTTCCGGGGCAGGGTCAGCATC | |
| TGGGCCATGGTGACCAGTGTGGACGGGAGCCAGCAGGTCGCGTTCGATGACTCCACCCCC | |
| GTGCAGAGGCAGCTGGTGGACATCCGGTACTCCAAGGACACGAGGAAGCAGTTCAAGCCG | |
| GGCCTGGCCTACGTGGGGAAGGTGGAGCTATCCTACCCCGATGGCAGCCCAGCTGAGGGG | |
| GTGACGGTCCAGATTAAGGCAGAGCTGACACCAAAGGATAACATCTACACCAGTGAAGTT | |
| GTGTCCCAGCGTGGACTAGTGGGGTTTGAAATCCCCTCCATCCCCACGTCAGCCCAGCAC | |
| GTGTGGCTGGAGACCAAGGTGATGGCACTGAACGGGAAGCCCGTGGGGGCTCAGTACCTG | |
| CCCAGCTACCTCTCCCTCGGCAGCTGGTACTCCCCCAGCCAGTGCTACCTGCAGCTGCAG | |
| CCACCCTCCCACCCACTGCAGGTTGGGGAAGAAGCCTATTTTTCTGTGAAGTCCACATGT | |
| CCCTGCAACTTTACCCTGTACTACGAGGTGGCTGCACGGGGCAATATTGTGCTATCGGGC | |
| CAGCAGCCTGCCCACACCACCCAGCAGCGAAGCAAGCGGGCGGCCCCTGCCCTGGAGAAA | |
| CCGATTCGTTTAACACACCTTTCTGAGACAGAGCCCCCACCAGCCCCAGAAGCTGAGGTC | |
| GACGTGTGTGTGACCTCTCTTCATCTGGCCGTGACCCCCAGCATGGTCCCCCTTGGTCGC | |
| CTGCTGGTCTTCTACGTCAGGGAGAATGGAGAAGGGGTCGCCGACAGCCTTCAGTTTGCA | |
| GTCGAGACCTTCTTCGAAAACCAGGTTTCAGTGACGTATTCAGCAAATGAGACCCAACCT | |
| GGGGAGGTTGTCGACCTGCGGATCAGGGCTGCAAGGGGCAGCTGTGTGTGCGTCGCCGCA | |
| GTTGATAAGAGTGTCTACCTGCTCAGGTCTGGGTTCCGGCTGACTCCTGCCCAGGTTTTC | |
| CAGGAACTGGAAGATTATGATGTTTCTGATTCCTTTGGCGTGTCCAGGGAGGATGGTCCT | |
| TTTTGGTGGGCTGGGCTGACGGCACAACGACGCCGGCGCTCCTCTGTCTTCCCGTGGCCT | |
| TGGGGCATCACCAAGGACTCTGGGTTTGCCTTCACCGAAACGGGACTGGTGGTGATGACC | |
| GACCGAGTGAGCCTGAACCACCGGCAGGACGGTGGCCTCTACACCGATGAGGCTGTCCCC | |
| GCTTTCCAGCCCCACACAGGGAGCCTGGTGGCAGTGGCTCCTTCCAGGCACCCCCCCAGA | |
| ACAGAGAAGAGAAAAAGGACTTTCTTCCCCGAAACATGGATTTGGCATTGTCTCAACATC | |
| AGTGACCCATCTGGTGAGGGGACACTCAGTGTGAAGGTCCCGGACTCCATCACCAGCTGG | |
| GTGGGTGAGGCCGTGGCCCTGTCCACCTCTCAGGGCTTAGGCATCGCCGAGCCCTCCCTG | |
| CTCAAGACCTTCAAGCCCTTCTTCGTGGACTTCATGCTCCCCGCTCTCATCATCCGTGGG | |
| GAGCAGGTCAAGATCCCGCTCAGTGTCTACAACTACATGGGCACCTGCGCTGAGGTGTAC | |
| ATGAAGCTCTCGGTTCCCAAGGGCATCCAGTTTGTTGGGCATCCTGGCAAACGCCATGTG | |
| ACCAAGAAGATGTGTGTGGCCCCCGGGGAGGCTGAGCCCATCTGGGTCGTTCTGTCCTTC | |
| AGCGACCTGGGACTCAACAACATCACGGCCAAAGCCCTTGCTTACGGAGACACAAATTGC | |
| TGCCGGGATGGGAGGTCCAGCAAACACCCTGAGGAGAATCACCCCGACAGGAGGGTCCCC | |
| ATCGGGGTGGATCACGTCAGGCGCAGTGTGATGGTTGAGGCGGAAGGAGTCCCCCGGGCG | |
| TACACCTACAGCGCATTCTTCTGTCCCAGTGAGAGAGTCCACATCTCCACCCCCAACAAG | |
| TATGAGTTCCAGTATGTGCAGCGGCCACTGCGCCTCACCCGCTTTGATGTGGCTGTGCGA | |
| GCTCACAATGATGCCCGTGTGGCCTTGTCTTCTGGGCCCCAGGACACAGCAGGCATGATC | |
| GAGATCGTCCTGGGGGGGCATCAGAACACCAGGTCATGGATCTCCACCAGCAAGATGGGA | |
| GAGCCCGTGGCCAGTGCACACACGGCCAAGATCCTCTCCTGGGATGAATTCAGAACATTC | |
| TGGATCAGCTGGCGTGGTGGCCTTATCCAGGTTGGCCATGGTCCAGAGCCATCCAATGAG | |
| TCTGTCATTGTGGCCTGGACCCTCCCGAGGCCACCAGAGGTCCAGTTCATTGGCTTTTCC | |
| ACCGGCTGGGGCTCCATGGGTGAATTCCGAATCTGGAGGAAGATGGAGGTGGACGAGAGC | |
| TACAGCGAGGCCTTCACCCTGGGGGTCCCACACGGCGCCATCCCTGGGTCTGAGCGAGCC | |
| ACCGCCTCCATCATCGGGGACGTCATGGGGCCAACCCTGAACCACCTCAACAACCTCCTG | |
| CGGCTGCCGTTTGGCTGTGGAGAGCAGAACATGATCCACTTTGCACCCAACGTCTTTGTC | |
| TTGAAGTATCTTCAGAAAACCCAGCAGCTCAGCCCTGAGGTGGAGAGAGAGACCACCGAC | |
| TACCTAGTACAAGGCTACCAGCGCCAGCTGACCTACAAGCGCCAGGATGGCTCCTACAGC | |
| GCGTTTGGGGAGCGGGACGCATCGGGGAGCATGTGGCTCACAGCCTTTGTCCTGAAGTCC | |
| TTCGCACAGGCTCGCAGCTTTATCTTCGTGGACCCCCGGGAGCTGGCTGCCGCCAAGAGC | |
| TGGATCATCCAGCAGCAGCAGGCCGATGGCTCCTTCCTGGCCGTGGGCAGGGTCCTGAAC | |
| AAGGACATCCAGGGTGGGATCCACGGCATTGTCCCGCTGACAGCCTACGTGGTGGTTGCT | |
| CTCCTGGAAACAGGCACAGCCTCAGAGGAGGAGAGAGGCTCCACTGACAAAGCGAGGCAC | |
| TTCCTCGAGTCTGCTGCGCCCCTGGCCATGGACCCTTATAGCTGTGCCCTGACTACCTAC | |
| GCGCTGACCCTGCTCCGCAGCCCGGCAGCCCCTGAGGCACTGCGCAAGCTCCGTAGCCTG | |
| GCCATCATGCGAGATGGGGTCACCCACTGGAGCCTGTCAAATTCCTGGGACGTGGACAAG | |
| GGCACATTCTTGAGCTTCAGTGACAGGGTCTCTCAGTCAGTGGTCTCGGCCGAGGTGGAA | |
| ATGACAGCCTACGCCCTTCTGACCTACACTCTGCTGGGTGACGTGGCTGCCGCCCTGCCT | |
| GTGGTGAAGTGGCTGTCCCAGCAGCGAAATGCACTTGGGGGCTTCTCCTCCACTCAGGAC | |
| ACCTGCGTGGCTCTGCAGGCCTTGGCTGAATATGCCATCTTGTCCTATGCTGGAGGCATC | |
| AACCTCACTGTCTCCCTGGCCTCCACCAACCTGGACTACCAGGAAACCTTCGAGCTGCAC | |
| AGGACCAACCAGAAGGTTCTGCAGACAGCAGCGATCCCCAGCCTCCCCACGGGGCTGTTT | |
| GTGAGTGCCAAGGGGGACGGCTGCTGCCTGATGCAGATTGATGTCACCTACAATGTGCCT | |
| GACCCGGTGGCCAAGCCAGCTTTCCAGCTGCTCGTAAGCCTCCAGGAGCCTGAGGCCCAG | |
| GGACGCCCGCCCCCCATGCCTGCCTCCGCAGCTGAGGGTTCCCGAGGAGACTGGCCCCCA | |
| GCTGACGATGATGACCCAGCGGCCGATCAGCATCACCAGGAATACAAGGTGATGCTGGAG | |
| GTGTGCACCAGGTGGCTGCATGCAGGGTCTTCCAATATGGCTGTCCTGGAGGTGCCCCTG | |
| CTGTCAGGCTTCCGGGCAGACATCGAGAGCCTGGAGCAGCTGCTCCTTGACAAGCACATG | |
| GGGATGAAGAGGTATGAAGTGGCTGGACGCCGAGTGCTCTTCTACTTTGATGAGATCCCC | |
| AGCCGGTGCCTGACGTGCGTGCGGTTCCGTGCTCTCCGGGAGTGCGTGGTGGGCAGGACG | |
| TCGGCGCTGCCAGTCTCCGTGTACGACTACTACGAACCCGCCTTCGAGGCCACTCGCTTC | |
| TACAACGTCAGCACGCACAGCCCACTCGCCCGGGAACTGTGCGCCGGACCCGCGTGCAAC | |
| GAAGTGGAGCGCGCCCCTGCCCGGGGCCCGGGCTGGTTCCCCGGCGAGTCGGGCCCTGCC | |
| GTGGCCCCTGAGGAGGGGGCGGCGATCGCGCGATGCGGCTGCGACCACGACTGCGGCGCC | |
| CAGGGGAACCCGGTGTGCGGCTCCGACGGGGTGGTCTACGCCAGCGCCTGCCGCCTGCGG | |
| GAGGCCGCCTGCCGCCAGGCCGCGCCCCTGGAGCCCGCGCCTCCCAGCTGCTGCGCCCTC | |
| GAGCAGCGGCTGCCGGCCTCGTCGTCCTCCACCTACGGGGATGACCTGGCTTCTGTGGCC | |
| CCGGGGCCTTTACAGCAGGACGTGAAGCTGAATGGAGCCGGCCTTGAGGTGGAGGACTCA | |
| GACCCTGAGCCTGAAGGGGAGGCGGAGGACAGGGTCACAGCCGGGCCTCGGCCTCCTGTG | |
| AGCAGCGGGAACCTGGAAAGCAGCACCCAGAGCGCCAGCCCGTTCCACAGATGGGGCCAG | |
| ACTCCGGCCCCTCAGAGACATAGTGGCCGGGTGGTGGGGGCCCACAGGCCAGGGCTTCTG | |
| AGCCCTGTCTTCGTCTACAGCCCAGCCTTTCAGAGTGGTGGGGAGGAGGGTTTATGGATG | |
| TCAAACACCTGCACCTTGAGATAATCCTACAACCACATGCAGTTGTGGGACCGCAGTTTG | |
| GTCCTGGGGACCATTCATACCCACACACCCAGCTTGTGCCTGTGGTTAACATCTCAGAAA | |
| ACTCTGGTAAATGATCACTCCAGGATATTGACACGAATACACGTTACTGATCTTACTCAC | |
| ATGTTCTGGGGTGCACATGAACTTTGTGTGTGCATGTGTGTGTGTGTGCATGTGTGTGTC | |
| CCGGGCACCTGACACCCCCAGCCCAGGGCTGCCCAAAGTTGGGCTGATCAGAGACATAGA | |
| CCCAATGAGGAGCCCAACAGTGGCCCTCCAACCCTCTGCCTTGCCCCCATAGTTCATGCC | |
| CCAGTGGTCTTTGAAACTGCCCTGTGCCACTCCCTGGAGTGAGCAGCGGTGTCTCTGTGT | |
| GTGTGTGTGTCTGTG | |

In a search of public sequence databases, the NOV11a nucleic acid sequence, located on chromsome 19 has 5574 of 5594 bases (99%) identical to a gb:GENBANK-ID:AB033109|acc:AB033109.1 mRNA from Homo sapiens (Homo sapiens mRNA for KIAA1283 protein, partial cds). Public nucleotide databases include all GenBank databases and the GeneSeq patent database.

The disclosed NOV11a polypeptide (SEQ ID NO:26) encoded by SEQ ID NO:25 has 1927 amino acid residues and is presented in Table 11B using the one-letter amino acid code. Signal P, Psort and/or Hydropathy results predict that NOV11a has a signal peptide and is likely to be localized at the plasma membrane with a certainty of 0.6400. The most likely cleavage site for a NOV11a peptide is between amino acids 25 and 26.

TABLE 11B
Encoded NOV11a protein sequence.

| | |
|---|---|
| MSGALLWPLLPLLLLLLSARDGVRAAQPQAPGYLIAAPSVFRAGVEEVISVTIFNSPREV | (SEQ ID NO:26) |
| TVQAQLVAQGEPVVQSQGAILDKGTIKLKHTVLSTSGISLLPILALLLGGRDLSSLFSLW | |
| PVLRYFQKQGQVPTGLRGQALLKVWGRGWQAEEGPLFHNQTSVTVDGRGASVFIQTDKPV | |
| YRPQHRVLISIFTVSPNLRPVNEKLEAYILDPRGSRMIEWRHLKPFCCGITNMSFPLSDQ | |
| PVLGEWFIFVEMQGHAYNKSFEVQKYVLPKFELLIDPPRYIQDLDACETGTVRARYTFGK | |
| PVAGALMINMTVNGVGYYSHEVGRPVLRTTKILGSRDFDICVRDMIPADVPEHFRGRVSI | |
| WAMVTSVDGSQQVAFDDSTPVQRQLVDIRYSKDTRKQFKPGLAYVGKVELSYPDGSPAEG | |
| VTVQIKAELTPKDNIYTSEVVSQRGLVGFEIPSIPTSAQHVWLETKVMALNGKPVGAQYL | |
| PSYLSLGSWYSPSQCYLQLQPPSHPLQVGEEAYFSVKSTCPCNFTLYYEVAARGNIVLSG | |
| QQPAHTTQQRSKRAAPALEKPIRLTHLSETEPPPAPEAEVDVCVTSLHLAVTPSMVPLGR | |
| LLVFYVRENGEGVADSLQFAVETFFENQVSVTYSANETQPGEVVDLRIRAARGSCVCVAA | |
| VDKSVYLLRSGFRLTPAQVFQELEDYDVSDSFGVSREDGPFWWAGLTAQRRRRSSVFPWP | |
| WGITKDSGFAFTETGLVVMTDRVSLNHRQDGGLYTDEAVPAFQPHTGSLVAVAPSRHPPR | |
| TEKRKRTFFPETWIWHCLNISDPSGEGTLSVKVPDSITSWVGEAVALSTSQGLGIAEPSL | |
| LKTFKPFFVDFMLPALIIRGEQVKIPLSVYNYMGTCAEVYMKLSVPKGIQFVGHPGKRHV | |
| TKKMCVAPGEAEPIWVVLSFSDLGLNNITAKALAYGDTNCCRDGRSSKHPEENHADRRVP | |
| IGVDHVRRSVMVEAEGVPRAYTYSAFFCPSERVHISTPNKYEFQYVQRPLRLTRFDVAVR | |
| AHNDARVALSSGPQDTAGMIEIVLGGHQNTRSWISTSKMGEPVASAHTAKILSWDEFRTF | |
| WISWRGGLIQVGHGPEPSNESVIVAWTLPRPPEVQFIGFSTGWGSMGEFRIWRKMEVDES | |
| YSEAFTLGVPHGAIPGSERATASIIGDVMGPTLNHLNNLLRLPFGCGEQNMIHFAPNVFV | |
| LKYLQKTQQLSPEVERETTDYLVQGYQRQLTYKRQDGSYSAFGERDASGSMWLTAFVLKS | |
| FAQARSFIFVDPRELAAAKSWIIQQQQADGSFLAVGRVLNKDIQGGIHGIVPLTAYVVVA | |
| LLETGTASEEERGSTDKARHFLESAAPLAMDPYSCALTTYALTLLRSPAAPEALRKLRSL | |
| AIMRDGVTHWSLSNSWDVDKGTFLSFSDRVSQSVVSAEVEMTAYALLTYTLLGDVAAALP | |
| VVKWLSQQRNALGGFSSTQDTCVALQALAEYAILSYAGGINLTVSLASTNLDYQETFELH | |
| RTNQKVLQTAAIPSLPTGLFVSAKGDGCCLMQIDVTYNVPDPVAKPAFQLLVSLQEPEAQ | |
| GRPPPMPASAAEGSRGDWPPADDDDPAADQHHQEYKVMLEVCTRWLHAGSSNMAVLEVPL | |
| LSGFRADIESLEQLLLDKHMGMKRYEVAGRRVLFYFDEIPSRCLTCVRFRALRECVVGRT | |
| SALPVSVYDYYEPAFEATRFYNVSTHSPLARELCAGPACNEVERAPARGPGWFPGESGPA | |
| VAPEEGAAIARCGCDHDCGAQGNPVCGSDGVVYASACRLREAACRQAAPLEPAPPSCCAL | |
| EQRLPASSSSTYGDDLASVAPGPLQQDVKLNGAGLEVEDSDPEPEGEAEDRVTAGPRPPV | |
| SSGNLESSTQSASPFHRWGQTPAPQRHSGRVVGAHRPGLLSPVFVYSPAFQSGGEEGLWM | |
| SNTCTLR | |

A search of sequence databases reveals that the NOV11a amino acid sequence has 1794 of 1797 amino acid residues (99%) identical to, and 1796 of 1797 amino acid residues (99%) similar to, the 1884 amino acid residue ptnr:SPTREMBL-ACC:Q9ULD7 protein from Homo sapiens (Human) (KIAA1283 PROTEIN). Public amino acid databases include the GenBank databases, SwissProt, PDB and PIR.

NOV11a is expressed in at least Adrenal Gland/Suprarenal gland, Bone Marrow, Brain, Heart, Kidney, Lung, Lymphoid tissue, Mammary gland/Breast, Pituitary Gland, Placenta, Prostate, Retina, Salivary Glands, Spleen, Thalamus, Thyroid. This information was derived by determining the tissue sources of the sequences that were included in the invention including but not limited to SeqCalling sources, Public EST sources, Literature sources, and/or RACE sources.

NOV11b

A disclosed NOV11b nucleic acid of 6069 nucleotides (also referred to as CG57488_{—}02) encoding a alpha-macroglobulin-like protein is shown in Table 11C. Putative untranslated regions upstream and/or downstream from the coding region, if any, are underlined, and the start and stop codons are in bold letters.

TABLE 11C
NOV11b nucleotide sequence.

| | |
|---|---|
| ATGAGCGGCGCCCTGCTCTGGCCGTTGCTCCCGCTCCTGCTCCTGCTGCTGTCGGCGCGG | (SEQ ID NO:27) |
| GACGGCGTGCGCGCCGCGCAGCCTCAGGCCCCGGGTTACTTGATTGCAGCTCCCTCTGTT | |
| TTTCGCGCGGGCGTGGAGGAAGTCATCAGCGTGACCATCTTTAACTCTCCAAGGGAAGTC | |
| ACGGTCCAGGCTCAGCTGGTGGCCCAGGGTGAGCCGGTGGTGCAGAGCCAGGGAGCCATC | |
| CTGGATAAAGGGACAATCAAACTCAAGGTGCCCACGGGCCTCCGGGGCCAAGCGCTTCTG | |
| AAAGTGTGGGGCCGCGGCTCGCAGGCGGAGGAGGGGCCCCTCTTTCACAACCACACCTCG | |
| GTGACCGTGGACGGCCGGGGCGCTTCTGTATTCATCCAGACGGACAAGCCTGTGTACAGA | |
| CCCCAGCACCGAGTGCTCATAAGCATCTTCACCGTCTCTCCAAATCTGAGGCCTGTCAAC | |
| GAGAAGCTGGAAGCCTACATCCTGGACCCCCGAGGCTCTCGGATGATAGAGTGGAGACAC | |
| TTAAAGCCGTTCTCCTGCGGCATCACCAACATGAGCTTCCCCTTGTCCGACCAGCCTGTG | |
| TTGGGAGAATGGTTCATTTTTGTTGAAATGCAAGGCCACGCGTACAACAAGTCTTTTGAA | |
| GTTCAGAAGTATGTGTTGCCCAAGTTTGAGCTTCTGATTGACCCGCCCCGGTATATCCAA | |
| GACCTGGACGCCTGTGAGACAGGCACTGTGCGGGCCAGGTATACCTTTGGGAAACCTGTG | |
| GCTGGTGCCTTAATGATCAACATGACTGTTAATGGTGTAGGGTACTACAGCCACGAGGTG | |
| GGACGCCCTGTCCTCAGAACAACCAAGATCCTCGGCTCCCGGGACTTCGACATCTGCGTG | |
| AGGGACATGATCCCAGCGGACGTCCCTGAGCACTTCCGGGCCAGGGTCAGCATCTGGGCC | |
| ATGGTGACCAGTGTGGACGGGAGCCAGCAGGTCGCGTTCGATGACTCCACCCCCGTGCAG | |
| AGGCAGCTGGTGGACATCCGGTACTCCAAGGACACGAGGAAGCAGTTCAAGCCGGGCCTG | |
| GCCTACGTGGGGAAGGTGGAGCTATCCTACCCCGATGGCAGCCCAGCTGAGGGGGTGACG | |
| GTCCAGATTAAGGCAGAGCTGACACCAAAGGATAACATCTACACCAGTGAAGTTGTGTCC | |
| CAGCGTGGACTAGTGGGGTTTGAAATCCCCTCCATCCCCACGTCAGCCCAGCACGTGTGG | |
| CTGGAGACCAAGGTGATGGCACTGAACGGGAAGCCCGTGGGGGCTCAGTACCTGCCCAGC | |
| TACCTCTCCCTCGGCAGCTGGTACTCCCCCAGCCAGTGCTACCTGCAGCTGCAGCCACCC | |
| TCCCACCCACTGCAGGTTGGGGAAGAAGCCTATTTTTCTGTGAAGTCCACATGTCCCTGC | |
| AACTTTACCCTGTACTACGAGGTGGCTGCACGGGGCAATATTGTGCTATCGGGCCAGCAG | |
| CCTGCCCACACCACCCAGCAGCGAAGCAAGCGGGCGGCCCCTGCCCTGGAGAAACCGATT | |
| CGTTTAACACACCTTTCTGAGACAGAGCCCCCACCAGCCCCAGAAGCTGAGGTCGACGTG | |
| TGTGTGACCTCTCTTCATCTGCCCGTGACCCCCAGCATGGTCCCCCTTGGTCGCCTGCTG | |
| GTCTTCTACGTCAGGGAGAATGGAGAAGGGGTCGCCGACAGCCTTCAGTTTGCAGTCGAG | |
| ACCTTCTTCGAAAACCAGGTTTCAGTGACGTATTCAGCAAATGAGACCCAACCTGGGGAG | |
| GTTGTCGACCTGCGGATCAGGGCTGCAAGGGGCAGCTGTGTGTGCGTCGCCGCAGTTGAT | |
| AAGAGTGTCTACCTGCTCAGGTCTGGCTTCCGGCTGACTCCTGCCCAGGTTTTCCAGGAA | |
| CTGGAAGATTATGATGTTTCTGATTCCTTTGGCGTGTCCAGGGAGGATGGTCCTTTTTGG | |
| TGGGCTGGGCTGACGGCACAACGACGCCGGCGCTCCTCTGTCTTCCCGTGGCCTTGGGGC | |
| ATCACCAAGGACTCTGGGTTTGCCTTCACCGAAACGGGACTGGTGCTGATGACCGACCGA | |
| GTGAGCCTGAACCACCGGCAGGACGGTGGCCTCTACACCGATGAGGCTGTCCCCGCTTTC | |
| CAGCCCCACACAGGGAGCCTGGTGGCAGTGGCTCCTTCCAGGCACCCCCCCAGAACAGAG | |
| AAGAGAAAAAGGACTTTCTTCCCCGAAACATGGATTTGGCATTGTCTCAACATCAGTGAC | |
| CCATCTGGTGAGGGGACACTCAGTGTGAAGGTCCCGGACTCCATCACCAGCTGGGTGGGT | |
| GAGGCCGTGGCCCTGTCCACCTCTCAGGGCTTAGGCATCGCCGAGCCCTCCCTGCTGAAG | |
| ACCTTCAAGCCCTTCTTCGTGGACTTCATGCTCCCCGCTCTCATCATCCGTGGGGAGCAG | |
| GTCAAGATCCCGCTCAGTGTCTACAACTACATGGGCACCTGCGCTGAGGTGTACATGAAG | |
| CTCTCGGTTCCCAAGGGCATCCAGTTTGTTGGGCATCCTGGCAAACGCCATGTGACCAAG | |
| AAGATGTGTGTGGCCCCCGGGGAGGCTGAGCCCATCTGGGTCGTTCTGTCCTTCAGCGAC | |
| CTGGGACTCAACAACATCACGGCCAAAGCCCTTGCTTACGGAGACACAAATTGCTGCCGG | |
| GATGGGAGGTCCAGCAAACACCCTGAGGAGAATCACGCCGACAGGAGGGTCCCCATCGGG | |
| GTGGATCACGTCAGGCGCAGTGTGATGGTTGAGGCGGAAGGAGTCCCCCGGGCGTACACC | |
| TACAGCGCATTCTTCTGTCCCAGTGAGAGAGTCCACATCTCCACCCCCAACAAGTATGAG | |
| TTCCAGTATGTGCAGCGGCCACTGCGCCTCACCCGCTTTGATGTGGCTGTGCGAGCTCAC | |
| AATGATGCCCGTGTGGCCTTGTCTTCTGGGCCCCAGGACACAGCAGGCATGATCGAGATC | |
| GTCCTGGGGGGGCATCAGAACACCAGGTCATGGATCTCCACCAGCAAGATGGGAGAGCCC | |
| GTGGCCAGTGCACACACGGCCAAGATCCTCTCCTGGGATGAATTCAGAACATTCTGGATC | |
| AGCTGGCGTGGTGGCCTTATCCAGGTTGGCCATGGTCCAGAGCCATCCAATGAGTCTGTC | |
| ATTGTGGCCTGGACCCTCCCGAGGCCACCAGAGGTCCAGTTCATTGGCTTTTCCACCGGC | |
| TGGGGCTCCATGGGTGAATTCCGAATCTGGAGGAAGATGGAGGTGGACGAGAGCTACAGC | |
| GAGGCCTTCACCCTGGGGGTCCCACACGCCGCCATCCCTGGGTCTGAGCGAGCCACCGCC | |
| TCCATCATCGGGGACGTCATGGGGCCAACCCTGAACCACCTCAACAACCTCCTGCGGCTG | |
| CCGTTTGGCTGTGGAGAGCAGAACATGATCCACTTTGCACCCAACGTCTTTGTCTTGAAG | |
| TATCTTCAGAAAACCCAGCAGCTCAGCCCTGAGGTGGAGAGAGAGACCACCGACTACCTA | |
| GTACAAGGCTACCAGCGCCAGCTGACCTACAAGCGCCAGGATGGCTCCTACAGCGCGTTT | |
| GGGGAGCGGGACGCATCGGGGAGCATGTGGCTCACAGCCTTTGTCCTGAAGTCCTTCGCA | |
| CAGGCTCGCAGCTTTATCTTCGTGGACCCCCGGGAGCTGGCTGCCGCCAAGAGCTGGATC | |
| ATCCAGCAGCAGCAGGCCGATGGCTCCTTCCTGGCCGTGGGCAGGGTCCTGAACAAGGAC | |
| ATCCAGGGTGGGATCCACGGCATTGTCCCGCTGACAGCCTACGTGGTGGTTGCTCTCCTG | |
| GAAACAGGCACAGCCTCAGAGGAGGAGAGAGGCTCCACTGACAAAGCGAGGCACTTCCTG | |
| GAGTCTGCTGCGCCCCTGGCCATGGACCCTTATAGCTGTGCCCTGACTACCTACGCGCTG | |
| ACCCTGCTCCGCAGCCCGGCAGCCCCTGAGGCACTGCGCAAGCTCCGTAGCCTGGCCATC | |
| ATGCGAGATGGGGTCACCCACTGGAGCCTGTCAAATTCCTGGGACGTGGACAAGGGCACA | |
| TTCTTGAGCTTCAGTGACAGGGTCTCTCAGTCAGTGGTCTCGGCCGAGGTGGAAATGACA | |
| GCCTACGCCCTTCTGACCTACACTCTGCTGGGTGACGTGGCTGCCGCCCTGCCTGTGGTG | |
| AAGTGGCTGTCCCAGCAGCGAAATGCACTTGGGGGCTTCTCCTCCACTCAGGACACCTGC | |
| GTGGCTCTGCAGGCCTTGGCTGAATATGCCATCTTGTCCTATGCTGGAGGCATCAACCTC | |
| ACTGTCTCCCTGGCCTCCACCAACCTGGACTACCAGGAAACCTTCGAGCTGCACAGGACC | |
| AACCAGAAGGTTCTGCAGACAGCAGCGATCCCCAGCCTCCCCACGGGGCTGTTTGTGAGT | |
| GCCAAGGGGGACGGCTGCTGCCTGATGCAGATTGATGTCACCTACAATGTGCCTGACCCG | |
| GTGGCCAAGCCAGCTTTCCAGCTGCTCGTAAGCCTCCAGGAGCCTGAGGCCCAGGGACGC | |
| CCGCCCCCCATGCCTGCCTCCGCAGCTGAGGGTTCCCGAGGAGACTGGCCCCCAGCTGAC | |
| GATGATGACCCAGCGGCCGATCAGCATCACCAGGAATACAAGGTGATGCTGGAGGTGTGC | |
| ACCAGGTGGCTGCATGCAGGGTCTTCCAATATGGCTGTCCTGGAGGTGCCCCTGCTGTCA | |
| GGCTTCCGGGCAGACATCCAGAGCCTGGAGCAGCTGCTCCTTGACAAGCACATGGGGATG | |
| AAGAGGTATGAAGTGGCTGGACGCCGAGTGCTCTTCTACTTTGATGAGATCCCCAGCCGG | |
| TGCCTGACGTGCGTCCGGTTCCGTGCTCTCCGGGAGTGCGTGGTGGGCAGGACGTCGGCG | |
| CTGCCAGTCTCCGTGTACGACTACTACGAACCCGCCTTCGAGGCCACTCGCTTCTACAAC | |
| GTCAGCACGCACAGCCCACTCGCCCGGGAACTGTGCGCCGGACCCGCGTGCAACGAAGTG | |
| GAGCGCGCCCCTGCCCGGGGCCCGGGCTGGTTCCCCGGCGAGTCGGGCCCTGCCGTGGCC | |
| CCTGAGGAGGGGGCGGCGATCGCGCGATGCGGCTGCGACCACGACTGCGGCGCCCAGGGG | |
| AACCCGGTGTGCGGCTCCGACGGGGTCGTCTACGCCAGCGCCTGCCGCCTGCGGGAGGCC | |
| GCCTGCCGCCAGGCCGCGCCCCTGGAGCCCGCGCCTCCCAGCTGCTGCGCCCTCGAGCAG | |
| CGGCTGCCGGCCTCGTCGTCCTCCACCTACGGGGATGACCTGGCTTCTGTGGCCCCGGGG | |
| CCTTTACAGCAGGACGTGAAGCTGAATGGAGCCGGCCTTGAGGTGGAGGACTCAGACCCT | |
| GAGCCTGAAGGGGAGGCGGAGGACAGGGTCACAGCCGGGCCTCGGCCTCCTGTGAGCAGC | |
| GGGAACCTGGAAAGCAGCACCCAGAGCGCCAGCCCGTTCCACAGATGGGGCCAGACTCCG | |
| GCCCCTCAGAGACATAGTGGCCGGGTGGTGGGGGCCCACAGGCCAGGGCTTCTGAGCCCT | |
| GTCTTCGTCTACACCCCAGCCTTTCAGAGTGGTGGGGAGGAGGGTTTATGGATGTCAAAC | |
| ACCTGCACCTTGAGATAATCCTACAACCACATGCAGTTGTGGGACCGCAGTTTGGTCCTG | |
| GGGACCATTCATACCCACACACCCAGCTTGTGCCTGTGGTTAACATCTCAGAAAACTCTG | |
| GTAAATGATCACTCCAGGATATTGACACGAATACACGTTACTGATCTTACTCACATGTTC | |
| TGGGGTGCACATGAACTTTGTGTGTGCATGTGTGTGTGTGTGCATGTGTGTGTCCCGGGC | |
| ACCTGACACCCCCAGCCCAGGGCTGCCCAAAGTTGGGCTGATCAGAGACATAGACCCAAT | |
| GAGGAGCCCAACAGTGGCCCTCCAACCCTCTGCCTTGCCCCCATAGTTCATGCCCCAGTG | |
| GTCTTTGAAACTGCCCTGTGCCACTCCCTGGAGTGAGCAGCGCTGTCTCTGTGTGTGTGT | |
| GTGTCTGTG | |

In a search of public sequence databases, the NOV11b nucleic acid sequence, located on chromsome 19 has 5815 of 5817 bases (99%) identical to a gb:GENBANK-ID:AB033109|acc:AB033109.1 mRNA from Homo sapiens (Homo sapiens mRNA for KIAA1283 protein, partial cds). Public nucleotide databases include all GenBank databases and the GeneSeq patent database.

The disclosed NOV11b polypeptide (SEQ ID NO:28) encoded by SEQ ID NO:27 has 1885 amino acid residues and is presented in Table D using the one-letter amino acid code. Signal P, Psort and/or Hydropathy results predict that NOV11b has a signal peptide and is likely to be localized at the plasma membrane with a certainty of 0.4600. The most likely cleavage site for a NOV11b peptide is between amino acids 25 and 26.

TABLE 11D
Encoded NOV11b protein sequence.

| | |
|---|---|
| MSGALLWPLLPLLLLLLSARDGVRAAQPQAPGYLIAAPSVFRAGVEEVISVTIFNSPREV | (SEQ ID NO:28) |
| TVQAQLVAQGEPVVQSQGAILDKGTIKLKVPTGLRGQALLKVWGRGWQAEEGPLFHNQTS | |
| VTVDGRGASVFIQTDKPVYRPQHRVLISIFTVSPNLRPVNEKLEAYILDPRGSRMIEWRH | |
| TVQAQLVAQGEPVVQSQGAILDKGTIKLKHTVLSTSGISLLPILALLLGGRDLSSLFSLW | |
| PVLRYFQKQGQVPTGLRGQALLKVWGRGWQAEEGPLFHNQTSVTVDGRGASVFIQTDKPV | |
| LKPFCCGITNMSFPLSDQPVLGEWFIFVEMQGHAYNKSFEVQKYVLPKFELLIDPPRYIQ | |
| DLDACETGTVRARYTFGKPVAGALMINMTVNGVGYYSHEVGRPVLRTTKILGSRDFDICV | |
| RDMIPADVPEHFRGRVSIWAMVTSVDGSQQVAFDDSTPVQRQLVDIRYSKDTRKQFKPGL | |
| AYVGKVELSYPDGSPAEGVTVQIKAELTPKDNIYTSEVVSQRGLVGFEIPSIPTSAQHVW | |
| LETKVMALNGKPVGAQYLPSYLSLGSWYSPSQCYLQLQPPSHPLQVGEEAYFSVKSTCPC | |
| NFTLYYEVAARGNIVLSGQQPAHTTQQRSKRAAPALEKPIRLTHLSETEPPPAPEAEVDV | |
| CVTSLHLAVTPSMVPLGRLLVFYVRENGEGVADSLQFAVETFFENQVSVTYSANETQPGE | |
| VVDLRIRAARGSCVCVAAVDKSVYLLRSGFRLTPAQVFQELEDYDVSDSFGVSREDGPFW | |
| WAGLTAQRRRRSSVFPWPWGITKDSGFAFTETGLVVMTDRVSLNHRQDGGLYTDEAVPAF | |
| QPHTGSLVAVAPSRHPPRTEKRKRTFFPETWIWHCLNISDPSGEGTLSVKVPDSITSWVG | |
| EAVALSTSQGLGIAEPSLLKTFKPFFVDFMLPALIIRGEQVKIPLSVYNYMGTCAEVYMK | |
| LSVPKGIQFVGHPGKRHVTKKMCVAPGEAEPIWVVLSFSDLGLNNITAKALAYGDTNCCR | |
| DGRSSKHPEENHADRRVPIGVDHVRRSVMVEAEGVPRAYTYSAFFCPSERVHISTPNKYE | |
| FQYVQRPLRLTRFDVAVRAHNDARVALSSGPQDTAGMIEIVLGGHQNTRSWISTSKMGEP | |
| VASAHTAKILSWDEFRTFWISWRGGLIQVGHGPEPSNESVIVAWTLPRPPEVQFIGFSTG | |
| WGSMGEFRIWRKMEVDESYSEAFTLGVPHGAIPGSERATASIIGDVMGPTLNHLNNLLRL | |
| PFGCGEQNMIHFAPNVFVLKYLQKTQQLSPEVERETTDYLVQGYQRQLTYKRQDGSYSAF | |
| GERDASGSMWLTAFVLKSFAQARSFIFVDPRELAAAKSWIIQQQQADGSFLAVGRVLNKD | |
| IQGGIHGIVPLTAYVVVALLETGTASEEERGSTDKARHFLESAAPLAMDPYSCALTTYAL | |
| TLLRSPAAPEALRKLRSLAIMRDGVTHWSLSNSWDVDKGTFLSFSDRVSQSVVSAEVEMT | |
| AYALLTYTLLGDVAAALPVVKWLSQQRNALGGFSSTQDTCVALQALAEYAILSYAGGINL | |
| TVSLASTNLDYQETFELHRTNQKVLQTAAIPSLPTGLFVSAKGDGCCLMQIDVTYNVPDP | |
| VAKPAFQLLVSLQEPEAQGRPPPMPASAAEGSRGDWPPADDDDPAADQHHQEYKVMLEVC | |
| TRWLHAGSSNMAVLEVPLLSGFRADIESLEQLLLDKHMGMKRYEVAGRRVLFYFDEIPSR | |
| CLTCVRFRALRECVVGRTSALPVSVYDYYEPAFEATRFYNVSTHSPLARELCAGPACNEV | |
| ERAPARGPGWFPGESGPAVAPEEGAAIARCGCDHDCGAQGNPVCGSDGVVYASACRLREA | |
| ACRQAAPLEPAPPSCCALEQRLPASSSSTYGDDLASVAPGPLQQDVKLNGAGLEVEDSDP | |
| EPEGEAEDRVTAGPRPPVSSGNLESSTQSASPFHRWGQTPAPQRHSGRVVGAHRPGLLSP | |
| VFVYSPAFQSGGEEGLWMSNTCTLR | |

A search of sequence databases reveals that the NOV11b amino acid sequence has 1882 of 1884 amino acid residues (99%) identical to, and 1883 of 1884 amino acid residues (99%) similar to, the 1884 amino acid residue ptnr:SPTREMBL-ACC:Q9ULD7 protein from Homo sapiens (Human) (KIAA1283 PROTEIN). Public amino acid databases include the GenBank databases, SwissProt, PDB and PIR.

NOV11b is expressed in at least Adrenal Gland/Suprarenal gland, Bone Marrow, Brain, Heart, Kidney, Lung, Lymphoid tissue, Mammary gland/Breast, Pituitary Gland, Placenta, Prostate, Retina, Salivary Glands, Spleen, Thalamus, Thyroid. This information was derived by determining the tissue sources of the sequences that were included in the invention including but not limited to SeqCalling sources, Public EST sources, Literature sources, and/or RACE sources.

NOV11e

A disclosed NOV11c nucleic acid of 6157 nucleotides (also referred to as CG57488_{—}03) encoding a alpha-macroglobulin-like protein is shown in Table 11E. Putative untranslated regions upstream and/or downstream from the coding region, if any, are underlined, and the start and stop codons are in bold letters.

TABLE 11E
NOV11C nucleotide sequence.

| | |
|---|---|
| GTGAGTAAGTGAGGGGACGATCCCCGGAAGGGATCGGGGCGGGTCGGGGTCCGGAGATGG | (SEQ ID NO:29) |
| GCGGAGCAGGCGTCCCGGGAGGGTGCGCCCAGGAGCGGGGCGAGCGGGGCGAGCGGGGCG | |
| GTCCCGGAGACGAGGCGGGTCCGGGGAGGGGGCTGGCCCGGGGCTGCCCCAGCTTGGCCG | |
| GGCGCGGAGCGGGGCGCATGGCGCCGGGCGCACTGCGCGGGGGCTGCGAACAAAGGGCCC | |
| CCGGCGGCGGCGCGAGGACGGCCGCGCTCGGACCCTGGCCCTGGCCCAGCCCTGGCCCGG | |
| CCCCCTCCCCAGGCGCGGCGCCCCCCAGGAGCCGAAAAATGAGCGGCGCCCTGCTCTGGC | |
| CGTTGCTCCCGCTCCTGCTCCTGCTGCTGTCGGCGCGGGACGGCGTGCGCGCCGCGCAGC | |
| CTCAGGCCCCGGGTTACTTGATTGCAGCTCCCTCTGTTTTTCGCGCGGGCGTGGAGGAAG | |
| TCATCAGCGTGACCATCTTTAACTCTCCAAGGGAAGTCACGGTCCAGGCTCAGCTGGTGG | |
| CCCAGGGTGAGCCGGTGGTGCAGAGCCAGGGAGCCATCCTGGATAAAGGGACAATCAAAC | |
| TCAAGGTGCCCACGGGCCTCCGGGGCCAAGCGCTTCTGAAAGTGTGGGGCCGCGGCTGGC | |
| AGGCGGAGGAGGGGCCCCTCTTTCACAACCAGACCTCGGTGACCGTGGACGGCCGGGGCG | |
| CTTCTGTATTCATCCAGACGGACAAGCCTGTGTACAGACCCCAGCACCGAGTGCTCATAA | |
| GCATCTTCACCGTCTCTCCAAATCTGAGGCCTGTCAACGAGAAGCTGGAAGCCTACATCC | |
| TGGACCCCCGAGGCTCTCGGATGATAGAGTGGAGACACTTAAAGCCGTTCTGCTGCGGCA | |
| TCACCAACATGAGCTTCCCCTTGTCCGACCAGCCTGTGTTGGGAGAATGGTTCATTTTTG | |
| TTGAAATGCAAGGCCACGCGTACAACAAGTCTTTTGAAGTTCAGAAGTATGTGTTGCCCA | |
| AGTTTGAGCTTCTGATTGACCCGCCCCGGTATATCCAAGACCTGGACGCCTGTGAGACAG | |
| GCACTGTGCGGGCCAGGTATACCTTTGGGAAACCTGTGGCTGGTGCCTTAACGATCAACA | |
| TGACTGTTAATGGTGTAGGGTACTACAGCCACGAGGTGGGACGCCCTGTCCTCAGAACAA | |
| CCAAGATCCTCGCCTCCCAGGACTTCGACATCTGCGTGAGGGACATGATCCCAGCGGACG | |
| TCCCTGAGCACTTCCGGGGCAGGGTCAGCATCTGGGCCATGGTGACCAGTGTGGACGGGA | |
| GCCAGCAGGTCGCGTTCGATGACTCCACCCCCGTGCAGAGGCAGCTGGTGGACATCCGGT | |
| ACTCCAAGGACACGAGGAAGCAGTTCAAGCCGGGCCTGGCCTACGTGGGGAAGGTGGAGC | |
| TATCCTACCCCGATGGCAGCCCAGCTGAGGGGGTGACGGTCCAGATTAAGGCAGAGCTGA | |
| CACCAAAGGATAACATCTACACCAGTGAAGTTGTGTCCCAGCGTGGACTAGTGGGGTTTG | |
| AAATCCCCTCCATCCCCACGTCAGCCCAGCACGTGTGGCTGGAGACCAAGGTGATGGCAC | |
| TGAACGGGAAGCCCGTGGGGGCTCAGTACCTGCCCAGCTACCTCTCCCTCGGCAGCTGGT | |
| ACTCCCCCAGCCAGTGCTACCTGCAGCTGCAGCCACCCTCCCACCCACTGCAGGTTGGGG | |
| AAGAAGCCTATTTTTCTGTGAAGTCCACATGTCCCTGCAACTTTACCCTGTACTACGAGG | |
| TGGCTGCACGGGGCAATATTGTGCTATCGGGCCAGCAGCCTGCCCACACCACCCAGCAGC | |
| GAAGCAAGCGGGCGGCCCCTCCCCTGGAGAAACCGATTCGTTTAACACACCTTTCTGAGA | |
| CAGAGCCCCCACCAGCCCCAGAAGCTGAGGTCGACGTGTGTGTGACCTCTCTTCATCTCG | |
| CCGTGACCCCCAGCATGGTCCCCCTTGGTCGCCTGCTGGTCTTCTACGTCAGGGAGAATC | |
| GAGAAGGGGTCGCCGACAGCCTTCAGTTTGCAGTCGAGACCTTCTTCGAAAACCAGGTTT | |
| CAGTGACGTATTCAGCAAATGAGACCCAACCTCGGGAGGTTGTCGACCTGCGGATCAGGG | |
| CTGCAAGGGGCACCTGTGTGTGCGTCGCCGCAGTTGATAAGAGTGTCTACCTGCTCAGGT | |
| CTGGGTTCCGGCTGACTCCTGCCCAGGTTTTCCAGGAACTGGAAGATTATGATGTTTCTG | |
| ATTCCTTTGGCGTGTCCAGGGAGGATGGTCCTTTTTGGTGGGCTGGGCTGACGGCACAAC | |
| GACGCCGGCGCTCCTCTGTCTTCCCGTGCCCTTGGGGCATCACCAAGGACTCTGGGTTTG | |
| CCTTCACCGAAACGGGACTGGTGGTGATGACCGACCGAGTGAGCCTGAACCACCGGCAGG | |
| ACGGTGGCCTCTACACCGATGAGGCTGTCCCCGCTTTCCAGCCCCACACAGGGAGCCTGG | |
| TGGCAGTGGCTCCTTCCAGGCACCCCCCCAGAACAGAGAAGAGAAAAAGGACTTTCTTCC | |
| CCGAAACATGGATTTGGCATTGTCTCAACATCAGTGACCCATCTGGTGAGGGGACACTCA | |
| GTGTGAAGGTCCCGGACTCCATCACCAGCTGGGTGGGTGAGGCCGTGGCCCTGTCCACCT | |
| CTCAGGGCTTAGGCATCGCCGAGCCCTCCCTGCTGAAGACCTTCAAGCCCTTCTTCGTGG | |
| ACTTCATGCTCCCCGCTCTCATCATCCGTGCGGAGCAGGTCAAGATCCCGCTCAGTGTCT | |
| ACAACTACATGGGCACCTGCGCTGAGGTGTACATGAAGCTCTCGGTTCCCAAGGGCATCC | |
| AGTTTGTTGGGCATCCTGGCAAACGCCATGTGACCAAGAAGATGTGTGTGGCCCCCGGGG | |
| AGGCTGAGCCCATCTGGGTCGTTCTGTCCTTCAGCGACCTGGGACTCAACAACATCACGG | |
| CCAAAGCCCTTGCTTACGGAGACACAAATTGCTGCCGGGATGGGAGGTCCAGCAAACACC | |
| CTGAGGAGAATCACGCCGACAGGAGGGTCCCCATCGGGGTGGATCACGTCAGGCGCAGTG | |
| TGATGGTTGAGGCGGAAGGAGTCCCCCGGGCGTACACCTACAGCGCATTCTTCTGTCCCA | |
| GTGAGAGAGTCCACATCTCCACCCCCAACAAGTATGAGTTCCAGTATGTGCAGCGGCCAC | |
| TGCGCCTCACCCGCTTTGATGTGGCTGTGCGAGCTCACAATGATGCCCGTGTGGCCTTGT | |
| CTTCTGGGCCCCAGGACACAGCAGGCATGATCGAGATCGTCCTGGGGGGGCATCAGAACA | |
| CCAGGTCATGGATCTCCACCAGCAAGATGGGAGAGCCCGTGGCCAGTGCACACACGGCCA | |
| AGATCCTCTCCTGGGATGAATTCAGAACATTCTGGATCAGCTGGCGTGGTGGCCTTATCC | |
| AGGTTGGCCATGGTCCAGAGCCATCCAATGAGTCTGTCATTGTGGCCTGGACCCTCCCGA | |
| GGCCACCAGAGGTCCAGTTCATTGGCTTTTCCACCGGCTGGGGCTCCATGGGTGAATTCC | |
| GAATCTGGAGGAAGATGGAGGTGGACGAGAGCTACAGCGAGGCCTTCACCCTGGGGGTCC | |
| CACACGGCGCCATCCCTGGGTCTGAGCGAGCCACCGCCTCCATCATCGGGGACGTCATGG | |
| GGCCAACCCTGAACCACCTCAACAACCTCCTGCGGCTGCCGTTTGGCTGTGGAGAGCAGA | |
| ACATGATCCACTTTGCACCCAACGTCTTTGTCTTGAAGTATCTTCAGAAAACCCAGCAGC | |
| TCAGCCCTGAGGTGGAGAGAGAGACCACCGACTACCTAGTACAAGGCTACCAGCGCCAGC | |
| TGACCTACAAGCGCCAGGATGGCTCCTACAGCGCGTTTGGGGAGCGGGACGCATCGGGGA | |
| GCATGTGGCTCACAGCCTTTGTCCTGAAGTCCTTCGCACAGGCTCGCAGCTTTATCTTCG | |
| TGGACCCCCGGGAGCTGGCTGCCGCCAAGAGCTGGATCATCCAGCAGCAGCAGGCCGATG | |
| GCTCCTTCCTGGCCGTGGGCAGGGTCCTGAACAAGGACATCCAGGGTGGGATCCACGGCA | |
| TTGTCCCGCTGACAGCCTACGTGGTGGTTGCTCTCCTGGAAACAGGCACAGCCTCAGAGG | |
| AGGAGAGAGGCTCCACTGACAAAGCGAGGCACTTCCTGGAGTCTGCTGCGCCCCTGGCCA | |
| TGGACCCTTATAGCTGTGCCCTGACTACCTACGCGCTGACCCTGCTCCGCAGCCCGGCAG | |
| CCCCTGAGGCACTGCGCAAGCTCCGTAGCCTGGCCATCATGCGAGATGGGGTCACCCACT | |
| GGAGCCTGTCAAATTCCTGGGACGTGGACAAGGGCACATTCTTGAGCTTCAGTGACAGGG | |
| TCTCTCAGTCAGTGGTCTCGGCCGAGGTGGAAATGACAGCCTACGCCCTTCTGACCTACA | |
| CTCTGCTGGGTGACGTGGCTGCCGCCCTGCCTGTGGTGAAGTGGCTGTCCCAGCAGCGAA | |
| ATGCACTTGGGGGCTTCTCCTCCACTCAGGACACCTGCGTGGCTCTGCAGGCCTTGGCTG | |
| AATATGCCATCTTGTCCTATGCTGGAGGCATCAACCTCACTGTCTCCCTGGCCTCCACCA | |
| ACCTGGACTACCAGGAAACCTTCGAGCTGCACAGGACCAACCAGAAGGTTCTGCAGACAG | |
| CAGCGATCCCCAGCCTCCCCACGGGGCTGTTTGTGAGTGCCAAGGGGGACGGCTGCTGCC | |
| TGATGCAGATTGATGTCACCTACAATGTGCCTGACCCGGTGGCCAAGCCAGCTTTCCAGC | |
| TGCTCGTAAGCCTCCAGGAGCCTGAGGCCCAGGGACGCCCGCCCCCCATGCCTGCCTCCG | |
| CAGCTGAGGGTTCCCGAGGAGACTGGCCCCCAGCTGACGATGATGACCCAGCGGCCGATC | |
| AGCATCACCAGGAATACAAGGTGATGCTGGAGGTGTGCACCAGGTGGCTGCATGCAGGGT | |
| CTTCCAATATGGCTGTCCTGGAGGTGCCCCTGCTGTCAGCCTTCCGGGCAGACATCGAGA | |
| GCCTGGAGCAGCTGCTCCTTGACAAGCACATGGGGATGAAGAGGTATGAAGTGGCTGGAC | |
| GCCGAGTGCTCTTCTACTTTGATGAGATCCCCAGCCGGTGCCTGACGTGCGTGCGGTTCC | |
| GTGCTCTCCGGGAGTGCGTGGTGGGCAGGACGTCGGCGCTGCCAGTCTCCGTGTACGACT | |
| ACTACGAACCCGCCTTCGAGGCCACTCGCTTCTACAACGTCAGCACGCACAGCCCACTCG | |
| CCCGGGAACTGTGCGCCGGACCCGCGTGCAACGAAGTGGAGCCCGCCCCTGCCCGGGGCC | |
| CGGGCTGGTTCCCCGGCGAGTCGGGCCCTGCCGTGGCCCCTGAGGAGGGGGCGGCGATCG | |
| CGCGATGCGGCTGCGACCACGACTGCGGCGCCCAGGGGAACCCGGTGTGCGGCTCCGACG | |
| GGGTGGTCTACGCCAGCGCCTGCCGCCTGCGGGAGGCCGCCTGCCGCCAGGCCGCGCCCC | |
| TGGAGCCCGCGCCTCCCAGCTGCTGCGCCCTCGAGCAGCGGCTGCCGGCCTCGTCGTCCT | |
| CCACCTACGGGGATGACCTGGCTTCTGTGGCCCCCGGGCCTTTACAGCAGGACGTGAAGC | |
| TGAATGGAGCCGGCCTTGACGTGGAGGACTCAGACCCTGAGCCTCAAGGGGAGGCGGAGG | |
| ACAGGGTCACAGCCGGGCCTCGGCCTCCTGTGAGCAGCGGGAACCTGGAAAGCAGCACCC | |
| AGAGCGCCAGCCCGTTCCACAGATGGGGCCAGACTCCGGCCCCTCAGAGACATAGTGGCC | |
| GGGTGGTGGGGGCCCACAGGCCAGGGCTTCTGAGCCCTGTCTTCGTCTACAGCCCAGCCT | |
| TTCAGAGTGGTGGGGAGGAGGGTTTATGGATGTCAAACACCTGCACCTTGAGATAATCCT | |
| ACAACCACATGCAGTTGTGGGACCGCAGTTTGGTCCTGGGGACCATTCATACCCACACAC | |
| CCAGCTTGTGCCTGTGGTTAACATCTCAGAAAACTCTGGTAAATGATCACTCCAGGATAT | |
| TGACACGAATACACGTTACTGATCTTACTCACATGTT | |

In a search of public sequence databases, the NOV11C nucleic acid sequence, located on chromsome 19 has 332 of 513 (64%) identical to a GENBANK-ID:GPIMSPB|acc:D84339.1 Cavia porcellus mRNA for murinoglobulin. Public nucleotide databases include all GenBank databases and the GeneSeq patent database.

The disclosed NOV11C polypeptide (SEQ ID NO:30) encoded by SEQ ID NO:29 has 1979 amino acid residues and is presented in Table 11F using the one-letter amino acid code. Signal P, Psort and/or Hydropathy results predict that NOV11C has no signal peptide and is likely to be localized at the plasma membrane with a certainty of 0.6000.

TABLE 11F
Encoded NOV11C protein sequence.

| | |
|---|---|
| MGGAGVPGGCAQERGERGERGGPGDEAGPGRGLARGCPSLAGRGAGRMAPGALRGGCEQR | (SEQ ID NO:30) |
| APGGGARTAALGPWPWPSPGPAPSPGAAPPRSRKMSGALLWPLLPLLLLLLSARDGVRAA | |
| QPQAPGYLIAAPSVFRAGVEEVISVTIFNSPREVTVQAQLVAQGEPVVQSQGAILDKGTI | |
| KLKVPTGLRGQALLKVWGRGWQAEEGPLFHNQTSVTVDGRGASVFIQTDKPVYRPQHRVL | |
| ISIFTVSPNLRPVNEKLEAYILDPRGSRMIEWRHLKPFCCGITNMSFPLSDQPVLGEWFI | |
| FVEMQGHAYNKSFEVQKYVLPKFELLIDPPRYIQDLDACETGTVRARYTFGKPVAGALTI | |
| NMTVNGVGYYSHEVGRPVLRTTKILGSQDFDICVRDMIPADVPEHFRGRVSIWAMVTSVD | |
| GSQQVAFDDSTPVQRQLVDIRYSKDTRKQFKPGLAYVGKVELSYPDGSPAEGVTVQIKAE | |
| LTPKDNIYTSEVVSQRGLVGFEIPSIPTSAQHVWLETKVMALNGKPVGAQYLPSYLSLGS | |
| WYSPSQCYLQLQPPSHPLQVGEEAYFSVKSTCPCNFTLYYEVAARGNIVLSGQQPAHTTQ | |
| QRSKRAAPALEKPIRLTHLSETEPPPAPEAEVDVCVTSLHLAVTPSMVPLGRLLVFYVRE | |
| NGEGVADSLQFAVETFFENQVSVTYSANETQPGEVVDLRIRAARGSCVCVAAVDKSVYLL | |
| RSGFRLTPAQVFQELEDYDVSDSFGVSREDGPFWWAGLTAQRRRRSSVFPWPWGITKDSG | |
| FAFTETGLVVMTDRVSLNHRQDGGLYTDEAVPAFQPHTGSLVAVAPSRHPPRTEKRKRTF | |
| FPETWIWHCLNISDPSGEGTLSVKVPDSITSWVGEAVALSTSQGLGIAEPSLLKTFKPFF | |
| VDFMLPALIIRGEQVKIPLSVYNYMGTCAEVYMKLSVPKGIQFVGHPGKRHVTKKMCVAP | |
| GEAEPIWVVLSFSDLGLNNITAKALAYGDTNCCRDGRSSKHPEENHADRRVPIGVDHVRR | |
| SVMVEAEGVPRAYTYSAFFCPSERVHISTPNKYEFQYVQRPLRLTRFDVAVRAHNDARVA | |
| LSSGPQDTAGMIEIVLGGHQNTRSWISTSKMGEPVASAHTAKILSWDEFRTFWISWRGGL | |
| IQVGHGPEPSNESVIVAWTLPRPPEVQFIGFSTGWGSMGEFRIWRKMEVDESYSEAFTLG | |
| VPHGAIPGSERATASIIGDVMGPTLNHLNNLLRLPFGCGEQNMIHFAPNVFVLKYLQKTQ | |
| QLSPEVERETTDYLVQGYQRQLTYKRQDGSYSAFGERDASGSMWLTAFVLKSFAQARSFI | |
| FVDPRELAAAKSWIIQQQQADGSFLAVGRVLNKDIQGGIHGIVPLTAYVVVALLETGTAS | |
| EEERGSTDKARHFLESAAPLAMDPYSCALTTYALTLLRSPAAPEALRKLRSLAIMRDGVT | |
| HWSLSNSWDVDKGTFLSFSDRVSQSVVSAEVEMTAYALLTYTLLGDVAAALPVVKWLSQQ | |
| RNALGGFSSTQDTCVALQALAEYAILSYAGGINLTVSLASTNLDYQETFELHRTNQKVLQ | |
| TAAIPSLPTGLFVSAKGDGCCLMQIDVTYNVPDPVAKPAFQLLVSLQEPEAQGRPPPMPA | |
| SAAEGSRGDWPPADDDDPAADQHHQEYKVMLEVCTRWLHAGSSNMAVLEVPLLSGFRADI | |
| ESLEQLLLDKHMGMKRYEVAGRRVLFYFDEIPSRCLTCVRFRALRECVVGRTSALPVSVY | |
| DYYEPAFEATRFYNVSTHSPLARELCAGPACNEVERAPARGPGWFPGESGPAVAPEEGAA | |
| IARCGCDHDCGAQGNPVCGSDGVVYASACRLREAACRQAAPLEPAPPSCCALEQRLPASS | |
| SSTYGDDLASVAPGPLQQDVKLNGAGLEVEDSDPEPEGEAEDRVTAGPRPPVSSGNLESS | |
| TQSASPFHRWGQTPAPQRHSGRVVGAHRPGLLSPVFVYSPAFQSGGEEGLWMSNTCTLR | |

A search of sequence databases reveals that the NOV11C amino acid sequence has 171 of 432 amino acid residues (39%) identical to, and 258 of 432 amino acid residues (58%) similar to, the guinea pig protein ptnr:SPTREMBL-ACC:Q60486 ALPHA-MACROGLOBULIN PRECURSOR—Cavia porcellus. Public amino acid databases include the GenBank databases, SwissProt, PDB and PIR.

The disclosed NOV11a polypeptide has homology to the amino acid sequences shown in the BLASTP data listed in Table 1G.

TABLE 11G
BLAST results for NOV11

| Gene Index/ | | Length | Identity | Positives | |
|---|---|---|---|---|---|
| Identifier | Protein/Organism | (aa) | (%) | (%) | Expect |
| gi\|6331358\|dbj\|BAA86597.1\| | KIAA1283 protein | 1884 | 1882/1926 | 1883/1926 | 0.0 |
| (AB033109) | [Homo sapiens] | | (97%) | (97%) | |
| gi\|15302736\|ref\|XP_ | KIAA1283 protein | 1711 | 1710/1711 | 1710/1711 | 0.0 |
| 050563.2\| | [Homo sapiens] | | (99%) | (99%) | |
| (XM_050563) | | | | | |
| gi\|18567969\|ref\|XP_ | Similar to | 837 | 665/732 | 667/732 | 0.0 |
| 095282.1\| | KIAA1283 protein | | (90%) | (90%) | |
| (XM_095282) | [Homo sapiens] | | | | |
| gi\|13928544\|dbj\|BAB47146.1\| | complement | 1732 | 220/611 | 313/611 | 4e−92 |
| (AB050668) | component C3 | | (36%) | (51%), | |
| | [Branchiostoma | | | | |
| | belcheri] | | | | |
| gi\|17975514\|ref\|NP_ | Thiolester | 1420 | 195/583 | 312/583 | 4e−85 |
| 523506.1\| | containing | | (33%) | (53%) | |
| (NM_078782) | protein II | | | | |
| | [Drosophila | | | | |
| | melanogaster] | | | | |

Tables 11H-I lists the domain descriptions from DOMAIN analysis results against NOV11. This indicates that the NOV11 sequence has properties similar to those of other proteins known to contain this domain.

TABLE 11H
Domain Analysis of NOV11

gnl|Pfam|pfam00207, A2M, Alpha-2-macroglobulin family. This family
includes the C-terminal region of the alpha-2-macroglobulin family.
CD-Length = 751 residues,
Score = 330 bits (847), Expect = 3e−91

TABLE 11I
Domain Analysis of NOV11

gnl|Smart|smart00280, KAZAL, Kazal type serine protease inhibitors;
Kazal type serine protease inhibitors and follistatin-like domains.
CD-Length = 46 residues, Score = 52.0 bits (123), Expect = 3e−07

NOV11 is a member of the alpha-macroglobulin family. Alpha-macroglobulin proteins are large extracellular glycoproteins that can bind to and often act as reservoirs of growth factors and extracellular enzymes (See Gonias et al., J Biol Chem Feb. 25, 2000;275(8):5826-31). Decreased level of these proteins in serum is often a sign of tissue damage (See Ruaux et al., Res Vet Sci 1999 August;67(1):83-7; Levine et al., J Pediatr Gastroenterol Nutr 1989 November;9(4): δ 17-20; Wiedermann et al., Neoplasma 1978;25(2):189-96). These proteins may also help defend the body against bacterial or parasitic infection (See Araujo-Jorge et al., Parasitol Res 1992;78(3):215-21).

The disclosed NOV11 nucleic acid of the invention encoding an alpha-macroglobulin-like protein includes the nucleic acid whose sequence is provided in Table 11A, 11C or 11E or a fragment thereof. The invention also includes a mutant or variant nucleic acid any of whose bases may be changed from the corresponding base shown in Table 11A, 11C or 11E while still encoding a protein that maintains its alpha-macroglobulin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV11 protein of the invention includes the alpha-macroglobulin-like protein whose sequence is provided in Table 11B, 11D or 11F. The invention also includes a mutant or variant protein any of whose residues may be changed from the corresponding residue shown in Table 11B, 11D or 11F while still encoding a protein that maintains its alpha-macroglobulin-like activities and physiological functions, or a functional fragment thereof. In the mutant or variant protein, up to about 1 percent of the residues may be so changed.

The invention further encompasses antibodies and antibody fragments, such as Fab or (Fab)₂, that bind immunospecifically to any of the proteins of the invention.

The above defined information for this invention suggests that this alpha-macroglobulin-like protein (NOV11) may function as a member of a “alpha-macroglobulin family”. Therefore, the NOV11 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV11 nucleic acids and proteins of the invention are useful in potential therapeutic applications implicated in diseases including but not limited to various pathologies and disorders as indicated below. For example, a cDNA encoding the alpha-macroglobulin-like protein (NOV11) may be useful in gene therapy, and the alpha-macroglobulin-like protein (NOV11) may be useful when administered to a subject in need thereof. By way of nonlimiting example, the compositions of the present invention will have efficacy for treatment of patients suffering from cancer, trauma, regeneration (in vitro and in vivo), viral/bacterial/parasitic infections, adrenoleukodystrophy, congenital adrenal hyperplasia, hemophilia, hypercoagulation, idiopathic thrombocytopenic purpura, autoimmune disease, allergies, immunodeficiencies, transplantation, graft versus host disease, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, systemic lupus erythematosus, autoimmune disease, asthma, emphysema, scleroderma, allergy, ARDS, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, diabetes, tuberous sclerosis, xerostomia, fertility, endocrine dysfunctions, growth and reproductive disorders, or other pathologies or conditions. The NOV11 nucleic acid encoding the alpha-macroglobulin-like protein of the invention, or fragments thereof, may further be useful in diagnostic applications, wherein the presence or amount of the nucleic acid or the protein are to be assessed.

NOV11 nucleic acids and polypeptides are further useful in the generation of antibodies that bind immuno-specifically to the novel NOV11 substances for use in therapeutic or diagnostic methods. These antibodies may be generated according to methods known in the art, using prediction from hydrophobicity charts, as described in the “Anti-NOVX Antibodies” section below. The disclosed NOV11 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV12

NOV12 includes two orphan transporter-like proteins disclosed below. The disclosed sequences have been named NOV12a and NOV12b.

NOV12a

A disclosed NOV12a nucleic acid of 2119 nucleotides (also referred to as CG57526-01) encoding an orphan receptor-like protein is shown in Table 12A. Putative untranslated regions upstream and/or downstream from the coding region, if any, are underlined, and the start and stop codons are in bold letters.

TABLE 12A
NOV12a nucleotide sequence.

| | |
|---|---|
| TGTGGTTTCCAAACGTCGGCAGAGGCTGGAGACGGCTCTCTAGTGCTGGGTGTGGAGTGA | (SEQ ID NO:31) |
| GGCACCACCCTCGCCCTGAAGCCTGGGGCACTCAGTCACCATGGCTCATGCCCCAGAACC | |
| AGACCCGGCCGCCAGCGACCTCGGGGATGAGAGGCCCAAGTGGGACAACAAGGCCCAGTA | |
| CCTCCTGAGCTGCATCGGGTTTGCCGTGGGGCTGGGGAACATTTGGCGGTTCCCATACCT | |
| GTGCCAGACCTATGGAGGAGGTGCCTTCCTCATCCCCTACGTCATCGCGCTGGTCTTCGA | |
| GGGGATCCCCATTTTCCACGTCGAGCTCGCCATCGGCCAGCGGCTGCGGAAGGGCAGCGT | |
| CGGCGTGTGGACGGCCATCTCCCCGTACCTCAGTGGAGTAGGTCTGGGCTGTGTCACGCT | |
| GTCCTTCCTGATCAGCCTGTACTACAACACCATCGTGGCGTGGGTGCTGTGGTACCTCCT | |
| CAACTCCTTCCAGCACCCGCTGCCCTGGAGCTCCTGCCCACCGGACCTCAACAGAACAGG | |
| TTTTGTGGAGGAGTGCCAGGGCAGCAGCGCCGTGAGCTACTTCTGGTACCGGCAGACACT | |
| GAACATCACAGCCGACATCAATGACAGTGGCTCCATCCAGTGGTGGCTGCTCATCTGCTT | |
| GGCAGCCTCCTGGGCAGTCGTGTACATGTGTGTCATCAGGGGCATTGAGACTACAGGGAA | |
| GGTGATTTACTTCACAGCTTTGTTCCCTTACCTGGTCCTGACCATCTTTCTCATCAGAGG | |
| GCTGACCCTGCCAGGGGCAACAAAAGGACTCATCTACTTGTTCACTCCCAACATGCACAT | |
| TCTCCAGAACCCCCGGGTGTGGCTGGACGCAGCCACCCAGATATTCTTCTCTCTGTCCCT | |
| GGCCTTCGGAGGACACATCGCTTTTGCAAGTTACAACTCGCCCAGGAGGAATGACTGCCA | |
| GAAGGATGCGGTGGTCATCGCCCTGGTCAACAGGATGACCTCCCTGTACGCGTCCATCGC | |
| TGTCTTCTCTGTCCTGGGGTTCAAAGCAACTAATGACCAGGAGCACTGCCTGGACAGGAA | |
| CATCCTCAGCCTCATCAACGACTTTGACTTCCCAGAGCAGAGCATCTCCAGGGACGACTA | |
| CCCAGCCGTCCTCATGCACCTGAACGCCACCTGGCCCAACAGGGTGGCCCAGCTCCCCCT | |
| GAAGGCCTGCCTCCTGGAAGACTTTCTGGATAAGAGTGCCTCGGGCCCGGGCCTGGCCTT | |
| CGTCGTCTTCACGGAGACCGACCTCCACATGCCGGGGGCTCCTGTGTGGGCCATGCTCTT | |
| CTTCGGGATGCTGTTCACCTTGGGGCTATCGACCATGTTCGGGACCGTGGAGCCCGTCAT | |
| CACACCCCTGCTGGACGTGGGGGTCCTGCCTAGATGGGTCCCCAAGGAGGCCCTGACTGG | |
| TCCAGGGCTGGTCTGCCTGGTCTGCTTCCTCTCCGCCACCTGCTTCACGCTGCAGTCTGG | |
| GAACTACTGGCTGGAGATTTTCGACAATTTTGCCGCTTCCCTGAACCTGCTCATGTTGGC | |
| CTTTCTCGAGGTTGTGGGTGTCGTTTATGTTTATGGAATGAAACGGTTCTCCGATGACAT | |
| TGCGTGGATGACCGGGAGGCGGCCCAGCCCCTACTGGCGGCTGACCTGGAGGGTGGTCAG | |
| TCCCCTGCTGCTGACCATCTTTGTGGCTTACATCATCCTCCTGTTCTGGAAGCCACTGAG | |
| ATACAAGGCCTGGAACCCCAAATACGAGCTGTTCCCCTCGCGTCAGGAGAAGCTCTACCC | |
| GGGCTGGGCGCGCGCCGCCTGTGTGCTGCTGTCCTTGCTGCCCGTGCTGTGGGTCCCGGT | |
| GGCCGCGCTTGCTCAGCTGCTCACCCGGCGGAGGCGGACGTGGAGGGACAGGGACGCGCG | |
| CCCAGACACGGACATGCGCCCGGACACGGACACGCGCCCAGACACGGACATGCGCCCGGA | |
| CACGGACATGCGCTGAAGCCGGCCGGAGCGGGGCCTGCATGGGCGGGTCTGTGGGGGGGC | |
| TTGGCCTGATGGTGGGCGGGGCCCCGCCCACAGGGCCGACCCCAATACACCAGCGACTCA | |
| ACCTTAAAAAAAAAAAAAA | |

In a search of public sequence databases, the NOV12a nucleic acid sequence, located on chromsome 5 has 1122 of 1396 bases (80%) identical to a gb:GENBANK-ID:AF075263|acc:AF075263.1 mRNA from Mus musculus (Mus musculus orphan transporter isoform A11 (Xtrp2) mRNA, alternatively spliced, complete cds. Public nucleotide databases include all GenBank databases and the GeneSeq patent database.

The disclosed NOV12a polypeptide (SEQ ID NO:32) encoded by SEQ ID NO:31 has 631 amino acid residues and is presented in Table 12B using the one-letter amino acid code. Signal P, Psort and/or Hydropathy results predict that NOV12a has a signal peptide and is likely to be localized to the plasma membrane with a certainty of 0.8000.

TABLE 12B
Encoded NOV12a protein sequence.

| | |
|---|---|
| MAHAPEPDPAASDLGDERPKWDNKAQYLLSCIGFAVGLGNIWRFPYLCQTYGGGAFLIPY | (SEQ ID NO:32) |
| VIALVFEGIPIFHVELAIGQRLRKGSVGVWTAISPYLSGVGLGCVTLSFLISLYYNTIVA | |
| WVLWYLLNSFQHPLPWSSCPPDLNRTGFVEECQGSSAVSYFWYRQTLNITADINDSGSIQ | |
| WWLLICLAASWAVVYMCVIRGIETTGKVIYFTALFPYLVLTIFLIRGLTLPGATKGLIYL | |
| FTPNMHILQNPRVWLDAATQIFFSLSLAFGGHIAFASYNSPRRNDCQKDAVVIALVNRMT | |
| SLYASIAVFSVLGFKATNDQEHCLDRNILSLINDFDFPEQSISRDDYPAVLMHLNATWPK | |
| RVAQLPLKACLLEDFLDKSASGPGLAFVVFTETDLHMPGAPVWAMLFFGMLFTLGLSTMF | |
| GTVEAVITPLLDVGVLPRWVPKEALTGPGLVCLVCFLSATCFTLQSGNYWLEIFDNFAAS | |
| LNLLMLAFLEVVGVVYVYGMKRFCDDIAWMTGRRPSPYWRLTWRVVSPLLLTIFVAYIIL | |
| LFWKPLRYKAWNPKYELFPSRQEKLYPGWARAACVLLSLLPVLWVPVAALAQLLTRRRRT | |
| WRDRDARPDTDMRPDTDTRPDTDMRPDTDMR | |

A search of sequence databases reveals that the NOV12a amino acid sequence has 460 of 602 amino acid residues (76%) identical to, and 528 of 602 amino acid residues (87%) similar to, the 615 amino acid residue ptnr:SPTREMBL-ACC:O88576 protein from Mus musculus (Mouse) (ORPHAN TRANSPORTER ISOFORM A12). Public amino acid databases include the GenBank databases, SwissProt, PDB and PIR.

NOV12a is expressed in at least Colon and Kidney. This information was derived by determining the tissue sources of the sequences that were included in the invention including but not limited to SeqCalling sources, Public EST sources, Literature sources, and/or PACE sources.

NOV12b

A disclosed NOV12b nucleic acid of 2039 nucleotides (also referred to as CG57526-02) encoding an orphan receptor-like protein is shown in Table 12C. Putative untranslated regions upstream and/or downstream from the coding region, if any, are underlined, and the start and stop codons are in bold letters.

TABLE 12C
NOV12b nucleotide sequence.

| | |
|---|---|
| TGTGGTTTCCAAACGTCGGCAGAGGCTGGAGACGGCTCTCTAGTGCTGGGTGTGGAGTGA | (SEQ ID NO:33) |
| GGCACCACCCTCGCCCTGAAGCCTGGGGCACTCAGTCACCATGGCTCATGCCCCAGAACC | |
| AGACCCGGCCGCCAGCGACCTCGGGGATGAGAGGCCCAAGTGGGACAACAAGGCCCAGTA | |
| CCTCCTGAGCTGCATCGGGTTTGCCGTGGGGCTGGGGAACATTTGGCGGTTCCCATACCT | |
| GTGCCAGACCTATGGAGGAGGTGCCTTCCTCATCCCCTACGTCATCGCGCTGGTCTTCGA | |
| GGGGATCCCCATTTTCCACGTCGAGCTCGCCATCGGCCAGCGGCTGCGGAAGGGCAGCGT | |
| CGGCGTGTGGACGGCCATCTCCCCGTACCTCAGTGGAGTAGGTCTGGGCTGTGTCACGCT | |
| GTCCTTCCTGATCAGCCTGTACTACAACACCATCGTGGCGTGGGTGCTGTGGTACCTCCT | |
| CAACTCCTTCCAGCACCCGCTGCCCTGGAGCTCCTGCCCACCGGACCTCAACAGAACAGG | |
| TTTTGTGGAGGAGTGCCAGGGCAGCAGCGCCGTGAGCTACTTCTGGTACCGGCAGACACT | |
| GAACATCACAGCCGACATCAATGACAGTGGCTCCATCCAGTGGTGGCTGCTCATCTGCTT | |
| GGCAGCCTCCTGGGCAGTCGTGTACATGTGTGTCATCAGGGGCATTGAGACTACAGGGAA | |
| GGTGATTTACTTCACAGCTTTGTTCCCTTACCTGGTCCTGACCATCTTTCTCATCAGAGG | |
| GCTGACCCTGCCAGGGGCAACAAAAGGACTCATCTACTTGTTCACTCCCAACATGCACAT | |
| TCTCCAGAACCCCCGGGTGTGGCTGGACGCAGCCACCCAGATATTCTTCTCTCTGTCCCT | |
| GGCCTTCGGAGGACACATCGCTTTTGCAAGTTACAACTCGCCCAGGAGGAATGACTGCCA | |
| GAAGGATGCGGTGGTCATCGCCCTGGTCAACAGGATGACCTCCCTGTACGCGTCCATCGC | |
| TGTCTTCTCTGTCCTGGGGTTCAAAGCAACTAATGACCAGGAGCACTGCCTGGACAGGAA | |
| CATCCTCAGCCTCATCAACGACTTTGACTTCCCAGAGCAGAGCATCTCCAGGGACGACTA | |
| CCCAGCCGTCCTCATGCACCTGAACGCCACCTGGCCCAAGAGGGTGGCCCAGCTCCCCCT | |
| GAAGGCCTGCCTCCTGGAAGACTTTCTGGATAAGAGTGCCTCGGGCCCGGGCCTGGCCTT | |
| CGTCGTCTTCACGGAGACCCACCTCCACATGCCGGGGGCTCCTGTGTGGGCCATGCTCTT | |
| CTTCGGGATGCTGTTCACCTTGGGGCTATCGACCATGTTCGGGACCGTGGAGGCGGTCAT | |
| CACACCCCTGCTGGACGTGGGGGTCCTGCCTAGATGGGTCCCCAAGGAGGCCCTGACTGG | |
| TCCAGGGCTGGTCTGCCTGGTCTGCTTCCTCTCCGCCACCTGCTTCACGCTGCAGTCTCG | |
| GAACTACTGGCTGGAGATTTTCGACAATTTTGCCGCTTCCCTGAACCTGCTCATGTTGGC | |
| CTTTCTCGAGGTTGTGGGTGTCGTTTATGTTTATGGAATGAAACGGTTCTGCGATGACAT | |
| TGCGTGGATGACCGGGAGGCGGCCCAGCCCCTACTGGCGGCTGACCTGGAGGGTGGTCAG | |
| TCCCCTGCTGCTGACCATCTTTGTGGCTTACATCATCCTCCTGTTCTGGAAGCCACTGAG | |
| ATACAAGGCCTGGAACCCCCAGGAGCTGTTCCCCTCGCGTCAGGAGAAGCTCTACCCGGG | |
| CTGGGCGCGCGCCGCCTGTGTGCTGCTGTCCTTGCTGCCCGTGCTGTGGGTCCCCGTGGC | |
| CGCGCTTGCTCAGCTGCTCACCCGGCGGAGGCGGACGTGGAGACAGGCGCATGCTGAGGC | |
| CGGGCTGGTGTTCCAGGACTTCGAGAAGCAGAGGCCTGGCGTGGGGATACAGTACCTGAT | |
| TCCAATGCTTTGCAACTTGCTCCAGACACTCTTCCGGTAGAAAAAGAGCCTGTTCTTTT | |

In a search of public sequence databases, the NOV12b nucleic acid sequence, located on chromsome 5 has 1122 of 1396 bases (80%) identical to a gb:GENBANK-ID:AF075263|acc:AF075263.1 mRNA from Mus musculus (Mus musculus orphan transporter isoform A11 (Xtrp2) mRNA, alternatively spliced, complete cds). Public nucleotide databases include all GenBank databases and the GeneSeq patent database.

The disclosed NOV12b polypeptide (SEQ ID NO:34) encoded by SEQ ID NO:33 has 639 amino acid residues and is presented in Table 12D using the one-letter amino acid code. Signal P, Psort and/or Hydropathy results predict that NOV12b has a signal peptide and is likely to be localized to the plasma membrane with a certainty of 0.8000.

TABLE 12D
Encoded NOV12b protein sequence.

| | |
|---|---|
| MAHAPEPDPAASDLGDERPKWDNKAQYLLSCIGFAVGLGNIWRFPYLCQTYCCCAFLIPY | (SEQ ID NO:34) |
| VIALVFEGIPIFHVELAIGQRLRKGSVGVWTAISPYLSGVGLGCVTLSFLISLYYNTTVA | |
| WVLWYLLNSFQHPLPWSSCPPDLNRTGFVEECQGSSAVSYFWYRQTLNITADINDSGSIQ | |
| WWLLICLAASWAVVYMCVIRGIETTGKVIYFTALFPYLVLTIFLIRGLTLPGATKGLIYL | |
| FTPNMHILQNPRVWLDAATQIFFSLSLAFGGHIAFASYNSPRRNDCQKDAVVIALVNRMT | |
| SLYASIAVFSVLGFKATNDQEHCLDRNILSLINDFDFPEQSISRDDYPAVLMHLNATWPK | |
| RVAQLPLKACLLEDFLDKSASGPGLAFVVFTETDLHMPGAPVWAMLFFGMLFTLGLSTMF | |
| GTVEAVITPLLDVGVLPRWVPKEALTGPGLVCLVCFLSATCFTLQSGNYWLEIFDNFAAS | |
| LNLLMLAFLEVVGVVYVYGMKRFCDDIAWMTGRRPSPYWRLTWRVVSPLLLTIFVAYIIL | |
| LFWKPLRYKAWNPQELFPSRQEKLYPGWARAACVLLSLLPVLWVPVAALAQLLTRRRRTW | |
| RQAHAEAGLVFQDFEKQRPGVGIQYLIPMLCNLLQTLFR | |

A search of sequence databases reveals that the NOV12b amino acid sequence has 465 of 613 amino acid residues (75%) identical to, and 534 of 613 amino acid residues (87%) similar to, the 615 amino acid residue ptnr:SPTREMBL-ACC:O88576 protein from Mus musculus (Mouse) (ORPHAN TRANSPORTER ISOFORM A12)(. Public amino acid databases include the GenBank databases, SwissProt, PDB and PIR.

NOV12b is expressed in at least Colon and Kidney. This information was derived by determining the tissue sources of the sequences that were included in the invention including but not limited to SeqCalling sources, Public EST sources, Literature sources, and/or RACE sources.

The disclosed NOV12 polypeptide has homology to the amino acid sequences shown in the BLASTP data listed in Table 12E.

TABLE 12E
BLAST results for NOV12

| Gene Index/ | | Length | Identity | Positives | |
|---|---|---|---|---|---|
| Identifier | Protein/Organism | (aa) | (%) | (%) | Expect |
| gi\|16550619\|dbj\|BAB71018.1\| | unnamed protein | 628 | 626/631 | 626/631 | 0.0 |
| (AK055798) | product [Homo | | (99%) | (99%) | |
| | sapiens] | | | | |
| gi\|3347922\|gb\|AAC27757.1\| | orphan | 615 | 460/603 | 528/603 | 0.0 |
| (AF075262) | transporter | | (76%) | (87%) | |
| | isoform A12 [Mus | | | | |
| | musculus] | | | | |
| gi\|8394204\|ref\|NP_058859.1\| | X transporter | 615 | 461/604 | 528/604 | 0.0 |
| (NM_017163) | protein 2 [Rattus | | (76%) | (87%) | |
| | norvegicus] | | | | |
| gi\|3347924\|gb\|AAC27758.1\| | orphan | 605 | 454/603 | 520/603 | 0.0 |
| (AF075263) | transporter | | (75%) | (85%) | |
| | isoform All [Mus | | | | |
| | musculus] | | | | |
| gi\|3347926\|gb\|AAC27759.1\| | orphan | 577 | 416/603 | 478/603 | 0.0 |
| (AF075264) | transporter | | (68%) | (78%) | |
| orphan | isoform B11 [Mus | | | | |
| | musculus] | | | | |

Table 12G lists the domain descriptions from DOMAIN analysis results against NOV12. This indicates that the NOV12 sequence has properties similar to those of other proteins known to contain this domain.

TABLE 12G
Domain Analysis of NOV12

gnl|Pfam|pfam00209, SNF, Sodium:neurotransmitter symporter family.
CD-Length = 534 residues, 92.5% aligned
Score = 469 bits (1207), Expect = 2e−133

A gene family encoding many Na(+)- and Cl(−)-dependent organic solute cotransporters has recently been recognized. Among the cotransporters that have been characterized are those for neurotransmitters, amino acids, and organic osmolytes. The cDNA is 2,354 bp long with an open reading frame of 1,845 bp. The 615 deduced amino sequence shows ROSIT to be most clearly related to two orphan cDNAs of this family isolated from brain. Northern analysis showed the mRNA is normally expressed in renal cortex but not in brain, heart, colon, liver, stomach, or skeletal muscle. Moreover, hypernatremic rats displayed a marked increase in mRNA levels in renal cortex, renal outer medulla, and perhaps intestine. Heterologous expression of the cRNA in Xenopus laevis oocytes failed to reveal the function of this gene product when analyzed with isotope fluxes or electrophysiological measurements using a wide variety of organic solutes. ROSIT is likely to be involved in kidney reclamation of an organic osmolyte or osmolyte precursor required for adaptation to hypertonic stress. (See Wasserman et al., Am J Physiol 1994 October;267(4 Pt 2):F688-94).

The disclosed NOV12 nucleic acid of the invention encoding a orphan receptor-like protein includes the nucleic acid whose sequence is provided in Table 12A or 12C or a fragment thereof. The invention also includes a mutant or variant nucleic acid any of whose bases may be changed from the corresponding base shown in Table 12A or 12C while still encoding a protein that maintains its orphan receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 20 percent of the bases may be so changed.

The disclosed NOV112 protein of the invention includes the orphan receptor-like protein whose sequence is provided in Table 12B or 12D. The invention also includes a mutant or variant protein any of whose residues may be changed from the corresponding residue shown in Table 12B or 12D while still encoding a protein that maintains its orphan receptor-like activities and physiological functions, or a functional fragment thereof. In the mutant or variant protein, up to about 24 percent of the residues may be so changed.

The invention further encompasses antibodies and antibody fragments, such as F_{ab} or (F_{ab})₂, that bind immunospecifically to any of the proteins of the invention.

The above defined information for this invention suggests that this orphan receptor-like protein (NOV12) may function as a member of a “orphan receptor family”. Therefore, the NOV12 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV12 nucleic acids and proteins of the invention are useful in potential therapeutic applications implicated in diseases including but not limited to various pathologies and disorders as indicated below. For example, a cDNA encoding the orphan receptor-like protein (NOV12) may be useful in gene therapy, and the orphan receptor-like protein (NOV12) may be useful when administered to a subject in need thereof. By way of nonlimiting example, the compositions of the present invention will have efficacy for treatment of patients suffering from cancer, trauma, regeneration (in vitro and in vivo), viral/bacterial/parasitic infections, Hirschsprung's disease, Crohn's Disease, appendicitis, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, or other pathologies or conditions. The NOV12 nucleic acid encoding the orphan receptor-like protein of the invention, or fragments thereof, may further be useful in diagnostic applications, wherein the presence or amount of the nucleic acid or the protein are to be assessed.

NOV12 nucleic acids and polypeptides are further useful in the generation of antibodies that bind immuno-specifically to the novel NOV12 substances for use in therapeutic or diagnostic methods. These antibodies may be generated according to methods known in the art, using prediction from hydrophobicity charts, as described in the “Anti-NOVX Antibodies” section below. The disclosed NOV12 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV13

A disclosed NOV13 nucleic acid of 1748 nucleotides (also referred to as CG-57570-01) encoding a cation transporter-like protein is shown in Table 13A. Putative untranslated regions upstream and/or downstream from the coding region, if any, are underlined, and the start and stop codons are in bold letters.

TABLE 13A
NOV13 nucleotide sequence.

| | |
|---|---|
| GTTCACCCCAAGACTAAGTTCTTTCCCAAGTTAGAGAAGAAGAGAGAAAGCAAAAAGAAG | (SEQ ID NO:35) |
| AGAGGAAAGTTCTCCCTTCCCCTCCTCCGTGCCTGTCATGTCCTCTAAGCCAGAGCCGAA | |
| GGACGTCCACCAACTGAACGGGACTGGCCCTTCTGCCTCTCCCTGCTCTTCAGATGGCCC | |
| AGGGAGAGAGCCCTTGGCTGGGACCTCAGAGTTCCTGGGGCCTGATGGGGCTGGGGTAGA | |
| GGTGGTGATTGAGTCTCGGGCCAACGCCAAGGGGGTTCGGGAGGAGGACGCCCTGCTGGA | |
| GAACGGGAGCCAGAGCAACGAAAGTGACGACGTCAGCACAGACCGTGGCCCTGCGCCACC | |
| TTCCCCGCTCAAGGAGACCTCCTTTTCCATCGGGCTGCAAGTACTGTTTCCATTCCTCCT | |
| GGCAGGCTTTGGGACCGTGGCTGCTGGCATGGTGTTGGACATCGTGCAGCACTGGGAAGT | |
| CTTCCAGAAGGTGACAGAGGTCTTCATCCTAGTGCCTGCGCTGCTGGGGCTCAAAGGGAA | |
| CCTGGAAATGACCCTGGCATCAAGGCTTTCCACTGCAGCGAGTATCAACATTGGACACAT | |
| GGACACACCCAAGGAGCTCTGGCGGATGATCACTGGGAACATGGCCCTCATCCAGGTGCA | |
| GGCCACGGTGGTGGGCTTCCTGGCGTCCATCGCAGCCGTCGTCTTTGGCTGGATCCCTGA | |
| TGGCCACTTCAGTATTCCGCACGCCTTCCTGCTCTGTGCTAGCAGCGTGGCCACAGCCTT | |
| CATTGCCTCCCTGGTACTGGGTATGATCATGATTGGAGTCATCATTGGCTCTCGCAAGAT | |
| TGGGATCAACCCAGACAATGTGGCCACACCCATTGCTGCCAGCCTGGGCGACCTCATCAC | |
| CTTGGCGCTGCTCTCAGGCATCAGCTGGGGACTCCTGACCTCTGCCCTCTCAGATCACTG | |
| GCGATACATCTACCCACTGGTGTGTGCTTTCTTTGTGGCCCTGCTGCCTGTCTGGGTGGT | |
| GCTGGCCCGACGAAGTCCAGCCACAAGGGAGGTGTTGTACTCGGGCTGGGAGCCTGTTAT | |
| CATTGCCATGGCCATCAGCAGTGTGGGAGGCCTCATCTTGGACAAGACTGTCTCAGACCC | |
| CAACTTTGCTGGGATGGCTGTCTTCACGCCTGTGATTAATGGTGTTGGGGGCAATCTGGT | |
| GGCAGTGCAGGCCAGCCGCATCTCCACCTTCCTGCACATGAATGGAATGCCCGGAGAGAA | |
| CTCTGAGCAAGCTCCTCGCCGCTGTCCCAGTCCTTGTACCACCTTCTTCAGCCCTGGTGT | |
| GAATTCTCGCTCAGCCCGGGTCCTCTTCCTCCTCGTGGTCCCAGGACACCTGGTGTTCCT | |
| CTACACCATCAGCTGTATGCAGGGCGGGCACACCACCCTCACACTCATCTTCATCATCTT | |
| CTATATGACAGCTGCACTGCTCCAGGTGCTGATTCTCCTGTACATCGCAGACTGGATGGT | |
| GCACTGGATGTGGGGCCGGGGCCTGGACCCGGACAACTTCTCCATCCCATACTTGACTGC | |
| TCTGGGGGACCTGCTTGGCACTGGGCTCCTAGCACTCAGCTTCCATGTTCTCTGGCTCAT | |
| AGGGGACCGAGACACGGATGTCGGGGACTAGCTTGGTCACTCAACATTTTCCCCATCCCT | |
| CTGCACTTTCTATTTGAAATTTTTCTTTTGTTCCCCTGTCCCTCCTCCACCCCACACTCC | |
| CACCTCTT | |

In a search of public sequence databases, the NOV13 nucleic acid sequence, located on chromsome 1 has 440 of 674 bases (65%) identical to a gb:GENBANK-ID:AK021925|acc:AK021925.1 mRNA from Homo sapiens (Homo sapiens cDNA FLJ11863 fis, clone HEMBA1006926). Public nucleotide databases include all GenBank databases and the GeneSeq patent database.

The disclosed NOV13 polypeptide (SEQ ID NO:36) encoded by SEQ ID NO:35 has 517 amino acid residues and is presented in Table 13B using the one-letter amino acid code. Signal P, Psort and/or Hydropathy results predict that NOV13 has a signal peptide and is likely to be localized at the plasma membrane with a certainty of 0.6000.

TABLE 13B
Encoded NOV13 protein sequence.

| | |
|---|---|
| MSSKPEPKDVHQLNGTGPSASPCSSDGPGREPLAGTSEFLGPDGAGVEVVIESRANAKGV | (SEQ ID NO:36) |
| REEDALLENGSQSNESDDVSTDRGPAPPSPLKETSFSIGLQVLFPFLLAGFGTVAAGMVL | |
| DIVQHWEVFQKVTEVFILVPALLGLKGNLEMTLASRLSTAASINIGHMDTPKELWRMITG | |
| NMALIQVQATVVGFLASIAAVVFGWIPDGHFSIPHAFLLCASSVATAFIASLVLGMIMIG | |
| VIIGSRKIGINPDNVATPIAASLGDLITLALLSGISWGLLTSALSDHWRYIYPLVCAFFV | |
| ALLPVWVVLARRSPATREVLYSGWEPVIIAMAISSVGGLILDKTVSDPNFAGMAVFTPVI | |
| NGVGGNLVAVQASRISTFLHMNGMPGENSEQAPRRCPSPCTTFFSPGVNSRSARVLFLLV | |
| VPGHLVFLYTISCMQGGHTTLTLIFIIFYMTAALLQVLILLYIADWMVHWMWGRGLDPDN | |
| FSIPYLTALGDLLGTGLLALSFHVLWLIGDRDTDVGD | |

A search of sequence databases reveals that the NOV13 amino acid sequence has 307 of 456 amino acid residues (67%) identical to, and 373 of 456 amino acid residues (81%) similar to, the 490 amino acid residue ptnr:TREMBLNEW-ACC:CAB66762 protein from Homo sapiens (Human) (HYPOTHETICAL 53.3 KDA PROTEIN). Public amino acid databases include the GenBank databases, SwissProt, PDB and PIR.

NOV13 is expressed in at least Liver, Pituitary Gland, Heart, Uterus, and B-cells. This information was derived by determining the tissue sources of the sequences that were included in the invention including but not limited to SeqCalling sources, Public EST sources, Literature sources, and/or RACE sources.

The disclosed NOV13 polypeptide has homology to the amino acid sequences shown in the BLASTP data listed in Table 13C.

TABLE 13C
BLAST results for NOV13

| Gene Index/ | | Length | Identity | Positives | |
|---|---|---|---|---|---|
| Identifier | Protein/Organism | (aa) | (%) | (%) | Expect |
| gi\|14149819\|ref\|NP_ | hypothetical protein | 490 | 305/457 | 372/457 | e−166 |
| 115524.1\| | DKFZp434K0427 | | (66%) | (80%) | |
| (NM_032148) | [Homo sapiens] | | | | |
| gi\|12053165\|emb\|CAB66762.1\| | hypothetical | 490 | 305/457 | 372/457 | e−166 |
| (AL136828) | protein [Homo | | (66%) | (80%), | |
| | sapiens] | | | | |
| gi\|15079232\|gb\|AAH11108.1\| | protein for | 488 | 235/438 | 306/438 | e−112 |
| AAH11108 | MGC: 18986) [Mus | | (53%) | (69%), | |
| (BC011108) | musculus] | | | | |
| gi\|14290540\|gb\|AAH09039.1\| | Similar to | 507 | 237/438 | 306/438 | e−111 |
| AAH09039 | hypothetical | | (54%) | (69%), | |
| (BC009039) | protein FLJ20473 | | | | |
| | [Homo sapiens] | | | | |
| gi\|12833620\|dbj\|BAB22598.1\| | homolog to CDNA | 462 | 230/445 | 304/445 | e−109 |
| (AK003140) | FLJ12718 FIS, | | (51%) | (67%), | |
| | CLONE | | | | |
| | NT2RP1001286˜putative | | | | |
| | [Mus | | | | |
| | musculus] | | | | |

Table 13D lists the domain descriptions from DOMAIN analysis results against NOV13. This indicates that the NOV13 sequence has properties similar to those of other proteins known to contain this domain.

TABLE 13D
Domain Analysis of NOV13

gnl|Pfam|pfam01769, MgtE, Divalent cation transporter. This region is
the integral membrane part of the eubacterial MgtE family of
magnesium transporters. Related regions are found also in
archaebacterial and eukaryotic proteins. All the archaebacterial and
eukaryotic examples have two copies of the region. This suggests that
the eubacterial examples may act as dimers. Members of this family
probably transport Mg2+ or other divalent cations into the cell. The
alignment contains two highly conserved Ds that may be involved in
cation binding (Bateman A unpubl.)
CD-Length = 131 residues, 99.2% aligned
Score = 66.6 bits (161), Expect = 3e−12

A gene family encoding many Na(+)- and Cl(−)-dependent organic solute cotransporters has recently been recognized. Among the cotransporters that have been characterized are those for neurotransmitters, amino acids, and organic osmolytes. The cDNA is 2,354 bp long with an open reading frame of 1,845 bp. The 615 deduced amino sequence shows ROSIT to be most clearly related to two orphan cDNAs of this family isolated from brain. Northern analysis showed the mRNA is normally expressed in renal cortex but not in brain, heart, colon, liver, stomach, or skeletal muscle. Moreover, hypernatremic rats displayed a marked increase in mRNA levels in renal cortex, renal outer medulla, and perhaps intestine. Heterologous expression of the cRNA in Xenopus laevis oocytes failed to reveal the function of this gene product when analyzed with isotope fluxes or electrophysiological measurements using a wide variety of organic solutes. ROSIT is likely to be involved in kidney reclamation of an organic osmolyte or osmolyte precursor required for adaptation to hypertonic stress. (See Wasserman et al., Am J Physiol 1994 October;267(4 Pt 2):F688-94).

Nramp1 regulates macrophage activation in infectious and autoimmune diseases. Nramp2 controls anaemia. Both are divalent cation (Fe(2+), Zn(2+), and Mn(2+)) transporters; Nramp2 a symporter of H(+) and metal ions, Nramp1 a H(+)/divalent cation antiporter. This provides a model for metal ion homeostasis in macrophages. Nramp2, localised to early endosomes, delivers extracellularly acquired divalent cations into the cytosol. Nramp1, localised to late endosomes/lysosomes, delivers divalent cations from the cytosol to phagolysosomes. Here, Fe(2+) generates antimicrobial hydroxyl radicals via the Fenton reaction. Zn(2-+) and Mn(2+) may also influence endosomal metalloprotease activity and phagolysosome fusion. The many cellular functions dependent on metal ions as cofactors may explain the multiple pleiotropic effects of Nramp1, and its complex roles in infectious and autoimmune disease. (See Blackwell et al., Microbes Infect 2000 March;2(3):317-21).

Mutations in the gene encoding the renal epithelial K(+) channel ROMK1 (Kir 1.1) is one of the causes for Bartter's syndrome, an autosomal recessive disease. It results in defective renal tubular transport in the thick ascending limb of the loop of Henle that leads to hypokalemic metabolic alkalosis and loss of salt. Two novel ROMK1 mutations, L220F/A 156V, have been described recently in a compound heterozygote patient demonstrating typical manifestations of Bartter's syndrome. Functional properties of these ROMK1 mutants were studied by coexpressing in Xenopus oocytes and by means of double electrode voltage clamp experiments. When both ROMK1 mutants were coexpressed no K(+) conductance could be detected. The same was found in oocytes expressing A156V-ROMK1 only or coexpressing wild type (wt) ROMK1 together with A156V-ROMK1. In contrast, K(+) conductances were indistinguishable from that of wt-ROMK1 when L220F-ROMK1 was expressed alone. Activation of protein kinase C signaling inhibited the conductance in both L220F-ROMK1 and wt-ROMK1 expressing oocytes. These effects were not seen in A156V-ROMK1 expressing oocytes.

The disclosed NOV13 nucleic acid of the invention encoding a cation transporter-like protein includes the nucleic acid whose sequence is provided in Table 13A or a fragment thereof. The invention also includes a mutant or variant nucleic acid any of whose bases may be changed from the corresponding base shown in Table 13A while still encoding a protein that maintains its cation transporter-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 35 percent of the bases may be so changed.

The disclosed NOV13 protein of the invention includes the cation transporter-like protein whose sequence is provided in Table 13B. The invention also includes a mutant or variant protein any of whose residues may be changed from the corresponding residue shown in Table B while still encoding a protein that maintains its cation transporter-like activities and physiological functions, or a functional fragment thereof. In the mutant or variant protein, up to about 33 percent of the residues may be so changed.

The invention further encompasses antibodies and antibody fragments, such as F_{ab} or (F_{ab})₂, that bind immunospecifically to any of the proteins of the invention.

The above defined information for this invention suggests that this cation transporter-like protein (NOV13) may function as a member of a “cation transporter family”. Therefore, the NOV13 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV13 nucleic acids and proteins of the invention are useful in potential therapeutic applications implicated in diseases including but not limited to various pathologies and disorders as indicated below. For example, a cDNA encoding the cation transporter-like protein (NOV13) may be useful in gene therapy, and the cation transporter-like protein (NOV13) may be useful when administered to a subject in need thereof. By way of nonlimiting example, the compositions of the present invention will have efficacy for treatment of patients suffering from cancer, trauma, regeneration (in vitro and in vivo), viral/bacterial/parasitic infections, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, endometriosis, fertility, Von Hippel-Lindau (VHL) syndrome, cirrhosis, endocrine dysfunctions, diabetes, obesity, growth and reproductive disorders, or other pathologies or conditions. The NOV13 nucleic acid encoding the cation transporter-like protein of the invention, or fragments thereof, may further be useful in diagnostic applications, wherein the presence or amount of the nucleic acid or the protein are to be assessed.

NOV13 nucleic acids and polypeptides are further useful in the generation of antibodies that bind immuno-specifically to the novel NOV13 substances for use in therapeutic or diagnostic methods. These antibodies may be generated according to methods known in the art, using prediction from hydrophobicity charts, as described in the “Anti-NOVX Antibodies” section below. The disclosed NOV13 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV14

A disclosed NOV14 nucleic acid of 5175 nucleotides (also referred to as CG57593-01) encoding a ABC transporter-like protein is shown in Table 14A. Putative untranslated regions upstream and/or downstream from the coding region, if any, are underlined, and the start and stop codons are in bold letters.

TABLE 14A
NOV14 nucicotide sequence.

| | |
|---|---|
| AATGTTCAAAGGCTTTCTGTAACTGAACTTTTTTTTTTTCTTTTTTCCCCTAGCTATTGC | (SEQ ID NO:37) |
| TCTGCAATATTTACTTTACCCTGTTAATGAACAGGACAAAATGGTTAAAAAAGAGATAAG | |
| CGTGCGTCAACAAATTCAGGCTCTTCTGTACAAGAATTTTCTTAAAAAATGGAGAATAAA | |
| AAGAGAGCTGGAGGAATGGACAATAACATTGTTTCTAGGGCTATATTTGTGCATCTTTTC | |
| GGAACACTTCAGAGCTACCCGTTTTCCTGAACAACCTCCTAAAGTCCTGGGAAGCGTGGA | |
| TCAGTTTAATGACTCTGGCCTGGTAGTGGCATATACACCAGTCAGTAACATAACACAAAG | |
| GATAATGAATAAGATGGCCTTGGCTTCCTTTATGAAAGGTAGAACAGTCATTGGGACACC | |
| AGATGAAGAGACCATGGATATAGAACTTCCAAAAAAATACCATGAAATGGTGGGAGTTAT | |
| ATTTAGTGATACTTTCTCATATCGCCTGAAGTTTAATTGGGCATATAGAATCCCAGTTAT | |
| AAAGGAGCACTCTGAATACACAGGTCACTGTTGGGCCATGCATGGTGAAATTTTTTGTTA | |
| CTTGGCAAAGTACTGGCTAAAAGGGTTTGTAGCTTTTCAAGCTGCAATTAATGCTGCAAT | |
| TATAGAAGTAAGTACAACAAATCATTCTGTAATGGAGGAGTTGACATCAGTTATTGGAAT | |
| AAATATGAAGATACCACCTTTCATTTCTAAGGGAGAAATTATGAATGAATGGTTTCATTT | |
| TACTTGCTTAGTTTCTTTCTCTTCTTTTATATACTTTGCATCATTAAATGTTGCAAGGGA | |
| AAGAGGAAAATTTAAGAAACTGATGACAGTGATGGGTCTCCGAGAGTCAGCATTCTGGCT | |
| CTCCTGGGGATTGACATACATTTGCTTCATCTTCATTATGTCCATTTTTATGGCTCTGGT | |
| CATAACATCAATCCCAATTGTATTTCATACTGGCTTCATGGTGATATTCACACTCTATAG | |
| CTTATATGGCCTTTCTTTGGTGTTGGCTTTCCTCATGAGTGTTTTAATAAGGAAACCTAT | |
| GCTCGCTGGTTTGGCTGGATTTCTCTTCACTGTATTTTGGGGATGTCTGGGATTCACTGT | |
| GTTATATAGACAACTTCCTTTATCTTTGGGATGGGTATTAAGTCTTCTTAGCCCTTTTGC | |
| CTTCACTGCTGGAATACAGATTACACACCTGGATAATTACTTAAGTGGTGTTATTTTTCC | |
| TGATCCCTCTGGGGATTCATACAAAATGATAGCCACTTTTTTCATTTTGGCATTTGATAC | |
| TCTTTTCTATTTGATATTCACATTATATTTTGAGCGAGTTTTACCTGGTAAGGGCCATGG | |
| GGATTCTCCATTATTTTTCCTTAAGTCCTCATTTTGGTCCAAACATCAAAATACTCATCA | |
| TGAAATCTTTGAGAATGAAATAAATCCTGAGCATTCCTCTGATGATTCTTTTGAACCGGT | |
| GTCTCCAGAATTCCATGGAAAAGAAGCCATAAGGATCAGAAATGTTATAAAAGAATATAA | |
| TGGAAAGACTGGAAAAGTAGAAGCATTGCAAATATTTTTTGACATATATGAAGGACAGAT | |
| CACTGCAATACTTGGGCATAATGGAGCTGGTAAATCAACACTGCTAAACATTCTTAGTGG | |
| ATTGTCTGTTTCTACAGAAGGTTCAGCCACTATTTATAATACTCAACTCTCTGAAATAAC | |
| TGACATGGAAGAAATTAGAAACAATATTGGATTTTGTCCACAGTTCAATTTTCAATTTGA | |
| CTTCCTCACTGTGAGAGAAAACCTCAGGGTATTTGCTAAAATAAAAGGGATTCAGCCAAA | |
| GGAAGTGGAACAAGAGGTATTGCTGCTAGATGAACCAACTGCTGGATTGGATCCCTTTTC | |
| AAGACACCGAGTGTGGAGCCTCCTGAAGGAGCATAAAGTAGACCGACTTATCCTCTTCAG | |
| TACCCAATTCATGGATGAGGCTGACATCTTGGCTGATAGGAAAGTATTTCTGTCTAATGG | |
| GAAGTTGAAATGTGCAGGATCATCTTTGTTTCTGAAGCGAAAGTGGGGTATTGGATATCA | |
| TTTAAGTTTACACAGGAATGAAATGTGTGACACAGAAAAAATCACATCCCTTATTAAGCA | |
| GCACATTCCTGATGCCAAGTTAACAACAGAAAGTGAAGAAAAACTTGTATATAGTTTGCC | |
| TTTGGAAAAAACGAACAAATTTCCAGATCTTTACAGTGACCTTGATAAGTGTTCTGACCA | |
| GGGCATAAGGAATTATGCTGTTTCAGTGACATCTCTGAATGAAGTATTCTTGAACCTAGA | |
| AGGAAAATCAGCAATTGATGAACCAGGTATATTTGACATTGGGAAACAAGAGAAAATACA | |
| TGTGACAAGAAATACTGGAGATGAGTCTGAAATGGAACAGGTTCTTTGTTCTCTTCCTGA | |
| AACAAGAAAGGCTGTCAGTAGTGCAGCTCTCTGGAGCCGACAAATCTATGCAGTGGCAAC | |
| ACTTCGCTTCTTAAAGTTAAGGCGTGAAAGGAGAGCTCTTTTGTGTTTGTTACTAGTACT | |
| TGGAATTGCTTTTATCCCCATCATTCTAGAGAAGATAATGTATAAAGTAACTCGTGAAAC | |
| TCATTGTTGGGAGTTTTCACCCAGTATGTATTTCCTTTCTCTGGAACAAATCCCGAAGAC | |
| GCCTCTTACCAGCCTGTTAATCGTTAATAATACAGGTTCAAATATTGAAGACCTCGTGCA | |
| TTCACTGAAGTGTCAGGATATAGTTTTGGAAATAGATGACTTTAGAAACAGAAATGGCTC | |
| AGATGATCCCTCCTACAATGGAGCCATCATAGTGTCTGGTGACCAGAAGGATTACAGATT | |
| TTCAGTTGCATGTAATACCAAGAAATCGAATTGTTTTCCGGTTCTTATGGGAATTGTTAG | |
| CAATGCCCTTATTGGAATTTTTAACTTCACAGAGCTTATTCAAATGGAGAGCACCTTCAT | |
| TTTTCGTGATGACATAGTGCTGGATCTTGGTTTTATAGATGGGTCCATATTTTTGTTGTT | |
| GATCACAAACTGCATTTCTCCTTATATTGGCATAACAGCATCAGTGATTATTAAAGTAAG | |
| AGGGAGAGAGAGGTCCCAGTTATGGATTTCAGGCCTCTGGCCTTCAGCATACTGGTGTGG | |
| ACAGGCTCTGGTGGACATTCCATTATACTTCTTGATTCTCTTTTCAATACATTTAATTTA | |
| CTACTTCATATTTCTGGGATTCCAGCTTTCATGGGAACTCATGTTTGTTTTGGTAAGTGA | |
| TCCATTATTTGCAGGTGGTATGCATAATTGGTTGTGCAGTTTCTCTTATATTCCTCACAT | |
| ATGTGCTTTCATTCATCTTTCGCAAGTGGAGAAAAAAAATGGCTTTTGGTCTTTTGGCTT | |
| TTTTATTGTAAGTATATATACATGCGTGCACATTTATATTAAATTTTATTTGCTTGATAA | |
| ATCTTTTTTGCCACTTGTGTTTACTTTTAACTTTTATTGTTCTTATGCTTTAATGCCTGT | |
| CTCTTGTAAATCTGTCTTACTTTTTGCTTTTTTATTTCTCCAAAAATATCTCCATATAGC | |
| TCCAATCCTTTTCCCTCTGTTTGCTTTTGTTAGTGTTATTTTCCTTTTTGTCATAAGGTG | |
| TCTGGAAATGAAGTATGGAAATGAAATAATGAATAAAGACCCAGTTTTCAGGATCTCTCC | |
| ACGGAGTAGAGAAACTCATCCCAATCCGGAAGAGCCCGAAGAAGAAGATGAAGATGTTCA | |
| AGCTGAAAGAGTCCAAGCAGCAAATGCACTCACTGCTCCAAACTTGGAGGAGGAACCAGT | |
| CATAACTGCAAGCTGTTTACACAAGGAATATTATGAGACAAAGAAAAGTTGCTTTTCAAC | |
| AAGAAAGAAGAAAATAGCCATCAGAAATGTTTCCTTTTGTGTTAAAAAAGGTGAAGTTTT | |
| GGGATTACTAGGACACAATGGAGCTGGTAAAAGTACTTCCATTAAAATGATAACTGGGTG | |
| CACAAAGCCAACTGCAGGAGTGGTAGTGGTGTTACAAGGCAGCAGAGCATCAGTAAGGCA | |
| ACAGCATGACAACAGCCTCAAGTTCTTGGGGTACTGCCCTCAGGAGAACTCACTGTGGCC | |
| CAAGCTTACAATGAAAGAGCACTTGGAGTTGTATGCAGCTGTGAAAGGACTGGGCAAAGA | |
| AGATGCTGCTCTCAGTATTTCAAGATTGGTGGAAGCTCTTAAGCTCCAGGAACAACTTAA | |
| GGCTCCTGTGAAAACTCTATCAGAGGGAATAAAGAGAAAGCTGTGCTTTGTGCTGAGCAT | |
| CCTGGGGAACCCATCAGTGGTGCTTCTAGATGAGCCGTTCACCGGGATGGACCCCGAGGG | |
| GCAGCAGCAAATGTGGCTTCAGGCTACCGTTAAAAACAAGGAGAGGGGCACCCTCTTGAC | |
| CACCCATTACATGTCAGAGGCTGAGGCTGTGTGTGACCGTATGGCCATGATGGTGTCAGG | |
| AACGCTAAGGAGGTGTATTGGTTCCATTCAACATCTGAAAAACAAGTTTGGTAGAGATTA | |
| TTTACTAGAAATAAAAATGAAAGAACCTACCCAGGTGGAAGCTCTCCACACAGAGATTTT | |
| GAAGCTTTTCCCACAGGCTGCTTGGCAGGAAAGATATTCCTCTTTAATGGCGTATAAGTT | |
| ACCTGTGGAGGATGTCCACCCTCTATCTCGGGCCTTTTTCAAGTTAGAGCGAGTGAAGCA | |
| GACCTTCAACCTGGAGGAATACAGCCTCTCTCAGGCTACCTTGGAGCAGGTGTTCTTAGA | |
| ACTCTGTAAAGAGCAGGAGCTCGGAAATGTTGATGATAAAATTGATACAACAGTTGAATG | |
| GAAACTTCTCCCACAGGAAGACCCTTAAAATGAAGAACCTCCTAACATTCAATTTTAGGT | |
| CCTACTACATTGTTAGTTTCCATAATTCTACAAGAATGTTTCCTTTTACTTCAGTTAACA | |
| AAAGAAAACATTTAATAAACATTCAATAATGATTACAGTTTTCATTTTTAAAAATTTAGG | |
| ATGAAGGAAACAAGGAAATATAGGGAAAAGTAGTAGACAAAATTAACAAAATCAGACATG | |
| TTATTCATCCCCAACATGGGTCTATTTTGTGCTTAAAAATAATTTAAAAATCATACAATA | |
| TTAGGTTGGTTATCG | |

In a search of public sequence databases, the NOV14 nucleic acid sequence, located on chromsome 17 has 1737 of 2520 bases (68%) identical to a gb:GENBANK-ID:AB020629|acc:AB020629.1 mRNA from Homo sapiens (Homo sapiens mRNA for KIAA0822 protein, complete cds). Public nucleotide databases include all GenBank databases and the GeneSeq patent database.

The disclosed NOV14 polypeptide (SEQ ID NO:38) encoded by SEQ ID NO:37 has 1595 amino acid residues and is presented in Table 14 using the one-letter amino acid code. Signal P, Psort and/or Hydropathy results predict that NOV14 has a signal peptide and is likely to be localized at the plasma membrane with a certainty of 0.8000. The most likely cleavage site for a NOV14 peptide is between amino acids 52 and 53.

TABLE 14B
Encoded NOV14 protein sequence.

| | |
|---|---|
| MVKKEISVRQQIQALLYKNFLKKWRIKRELEEWTITLFLGLYLCIFSEHFRATRFPEQPP | (SEQ ID NO:38) |
| KVLGSVDQFNDSGLVVAYTPVSNITQRIMNKMALASFMKGRTVIGTPDEETMDIELPKKY | |
| HEMVGVIFSDTFSYRLKFNWGYRIPVIKEHSEYTGHCWAMHGEIFCYLAKYWLKGFVAFQ | |
| AAINAAIIEVSTTNHSVMEELTSVIGINMKIPPFISKGEIMNEWFHFTCLVSFSSFIYFA | |
| SLNVARERGKFKKLMTVMGLRESAFWLSWGLTYICFIFIMSIFMALVITSIPIVFHTGFM | |
| VIFTLYSLYGLSLVLAFLMSVLIRKPMLAGLAGFLFTVFWGCLGFTVLYRQLPLSLGWVL | |
| SLLSPFAFTAGIQITHLDNYLSGVIFPDPSGDSYKMIATFFILAFDTLFYLIFTLYFERV | |
| LPGKGHGDSPLFFLKSSFWSKHQNTHHEIFENEINPEHSSDDSFEPVSPEFHGKEAIRIR | |
| NVIKEYNGKTGKVEALQIFFDIYEGQITAILGHNGAGKSTLLNILSGLSVSTEGSATIYN | |
| TQLSEITDMEEIRKNIGFCPQFNFQFDFLTVRENLRVFAKIKGIQPKEVEQEVLLLDEPT | |
| AGLDPFSRHRVWSLLKEHKVDRLILFSTQFMDEADILADRKVFLSNGKLKCAGSSLFLKR | |
| KWGIGYHLSLHRNEMCDTEKITSLIKQHIPDAKLTTESEEKLVYSLPLEKTNKFPDLYSD | |
| LDKCSDQGIRNYAVSVTSLNEVFLNLEGKSAIDEPGIFDIGKQEKIHVTRNTGDESEMEQ | |
| VLCSLPETRKAVSSAALWSRQIYAVATLRFLKLRRERRALLCLLLVLGIAFIPIILEKIM | |
| YKVTRETHCWEFSPSMYFLSLEQIPKTPLTSLLIVNNTGSNIEDLVHSLKCQDIVLEIDD | |
| FRNRNGSDDPSYNGAIIVSGDQKDYRFSVACNTKKSNCFPVLMGIVSNALIGIFNFTELI | |
| QMESTFIFRDDIVLDLGFIDGSIFLLLITNCISPYIGITASVIIKVRGRERSQLWISGLW | |
| PSAYWCGQALVDIPLYFLILFSIHLIYYFIFLGFQLSWELMFVLVSDPLFAGGMHNWLCS | |
| FSYIPHICAFIHLSQVEKKNGFWSFGFFIVSIYTCVHIYIKFYLLDKSFLPLVFTFNFYC | |
| SYALMPVSCKSVLLFAFLFLQKYLHIAPILFPLFAFVSVIFLFVIRCLEMKYGNEIMNKD | |
| PVFRISPRSRETHPNPEEPEEEDEDVQAERVQAANALTAPNLEEEPVITASCLHKEYYET | |
| KKSCFSTRKKKIAIRNVSFCVKKGEVLGLLGHNGAGKSTSIKMITGCTKPTAGVVVVLQG | |
| SRASVRQQHDNSLKFLGYCPQENSLWPKLTMKEHLELYAAVKGLGKEDAALSISRLVEAL | |
| KLQEQLKAPVKTLSEGIKRKLCFVLSILGNPSVVLLDEPFTGMDPEGQQQMWLQATVKNK | |
| ERGTLLTTHYMSEAEAVCDRMAMMVSGTLRRCIGSIQHLKNKFGRDYLLEIKMKEPTQVE | |
| ALHTEILKLFPQAAWQERYSSLMAYKLPVEDVHPLSRAFFKLERVKQTFNLEEYSLSQAT | |
| LEQVFLELCKEQELGNVDDKIDTTVEWKLLPQEDP | |

A search of sequence databases reveals that the NOV14 amino acid sequence has 747 of 1321 amino acid residues (56%) identical to, and 951 of 1321 amino acid residues (71%) similar to, the 1581 amino acid residue ptnr:SPTREMBL-ACC:O94911 protein from Homo sapiens (Human) (KIAA0822 PROTEIN). Public amino acid databases include the GenBank databases, SwissProt, PDB and PIR.

NOV14 is expressed in at least epidermis. This information was derived by determining the tissue sources of the sequences that were included in the invention including but not limited to SeqCalling sources, Public EST sources, Literature sources, and/or RACE sources.

The disclosed NOV14 polypeptide has homology to the amino acid sequences shown in the BLASTP data listed in Table 14C.

TABLE 14C
BLAST results for NOV14

| Gene Index/ | | Length | Identity | Positives | |
|---|---|---|---|---|---|
| Identifier | Protein/Organism | (aa) | (%) | (%) | Expect |
| gi\|18587267\|ref\|XP_ | ATP-binding | 1543 | 1391/1563 | 1423/1563 | 0.0 |
| 085647.1\| | cassette, sub- | | (88%) | (90%) | |
| (XM_085647) | family A (ABC1), | | | | |
| | member 10 [Homo | | | | |
| | sapiens] | | | | |
| gi\|17933760\|ref\|NP_ | ATP-binding | 1543 | 1387/1563 | 1420/1563 | 0.0 |
| 525021.1\| | cassette, sub- | | (88%) | (90%), | |
| (NM_080282) | family A (ABC1), | | | | |
| | member 10 [Homo | | | | |
| | sapiens] | | | | |
| gi\|6005701\|ref\|NP_009099.1\| | ATP-binding | 1581 | 943/1604 | 1183/1604 | 0.0 |
| (NM_007168) | cassette, sub- | | (58%) | (72%) | |
| | family A (ABC1), | | | | |
| | member 8 [Homo | | | | |
| | sapiens] | | | | |
| gi\|17933764\|ref\|NP_ | ATP-binding | 1617 | 951/1659 | 1173/1659 | 0.0 |
| 525023.1\| | cassette, sub- | | (57%) | (70%), | |
| (NM_080284) | family A (ABC1), | | | | |
| | member 6 [Homo | | | | |
| | sapiens] | | | | |
| gi\|18587273\|ref\|XP_ | ATP-binding | 1624 | 940/1653 | 1189/1653 | 0.0 |
| 085646.1\| | cassette, sub- | | (56%) | (71%), | |
| (XM_085646) | family A (ABC1), | | | | |
| | member 9 [Homo | | | | |
| | sapiens] | | | | |

Table 14D lists the domain descriptions from DOMAIN analysis results against NOV 14. This indicates that the NOV14 sequence has properties similar to those of other proteins known to contain this domain.

TABLE 14D
Domain Analysis of NOV14

gnl|Pfam|pfam00005, ABC_tran, ABC transporter. ABC transporters
for a large family of proteins responsible for translocation of a variety
of compounds across biological membranes. ABC transporters are the
largest family of proteins in many completely sequenced bacteria. ABC
transporters are composed of two copies of this domain and two copies
of a transmembrane domain pfam00664. These four domains may
belong to a single polypeptide, or belong in different polypeptide
chains.
CD-Length = 183 residues, 98.4% aligned
Score = 132 bits (331), Expect = 2e−31

TABLE 14E
Domain Analysis of NOV14

gnl|Smart|smart00382, AAA, ATPases associated with a variety of
cellular activities; AAA - ATPases associated with a variety of cellular
activities. This profile/alignment only detects a fraction of
this vast family. The poorly conserved N-terminal helix is missing
from the alignment.
CD-Length = 151 residues, 98.7% aligned
Score = 49.3 bits (116), Expect = 2e−06

ABCAI, a member of the ATP binding cassette family, mediates the efflux of excess cellular lipid to HDL and is defective in Tangier disease. The apolipoprotein acceptor specificity for lipid efflux by ABCAI was examined in stably transfected Hela cells, expressing a human ABCAI-GFP fusion protein. ApoA-I and all of the other exchangeable apolipoproteins tested (apoA-II, apoA-IV, apoC-I, apoC-II, apoC-III, apoE) showed greater than a threefold increase in cholesterol and phospholipid efflux from ABCAI-GFP transfected cells compared to control cells. Expression of ABCAI in Hela cells also resulted in a marked increase in specific binding of both apoA-I (Kd=0.60 ?g/mL) and apoA-II (Kd=0.58 ?g/mL) to a common binding site. In summary, ABCAI-mediated cellular binding of apolipoproteins and lipid efflux is not specific for only apoA-I but can also occur with other apolipoproteins that contain multiple amphipathic helical domains. (See Remaley et al., Biochem Biophys Res Commun Jan. 26, 2001;280(3):818-823).

The molecular mechanisms regulating the amount of dietary cholesterol retained in the body, as well as the body's ability to exclude selectively other dietary sterols, are poorly understood. An average western diet will contain about 250-500 mg of dietary cholesterol and about 200-400 mg of non-cholesterol sterols. About 50-60% of the dietary cholesterol is absorbed and retained by the normal human body, but less than 1% of the non-cholesterol sterols are retained. Thus, there exists a subtle mechanism that allows the body to distinguish between cholesterol and non-cholesterol sterols. In sitosterolemia, a rare autosomal recessive disorder, affected individuals hyperabsorb not only cholesterol but also all other sterols, including plant and shellfish sterols from the intestine. The major plant sterol species is sitosterol; hence the name of the disorder. Consequently, patients with this disease have very high levels of plant sterols in the plasma and develop tendon and tuberous xanthomas, accelerated atherosclerosis, and premature coronary artery disease. The STSL locus was mapped to human chromosome 2p21 (ref. 4) and was localized it to a region of less than 2 cM bounded by markers D2S2294 and D2S2291. A new member of the ABC transporter family, ABCG5, is mutant in nine unrelated sitosterolemia patients. (See Lee et al., Nat Genet 2001 January;27(1):79-83).

Pseudoxanthoma elasticum (PXE) is an inherited systemic disorder of connective tissue, characterized by progressive calcification of the elastic fibers in the eye, the skin, and the cardiovascular system. The PXE locus has been mapped to chromosome 16p13.1, and was recently further refined to a 500 kb-region, containing four candidate genes: MRP1 (ABCC1), MRP6 (ABCC6), pM5, and two copies of an unknown gene, the later subsequently found to be identical to the gene encoding the Nuclear Pore Interacting Protein (NPIP). In a comprehensive mutational screening, the entire coding region of the pM5, MRP1, and NPIP genes were analyzed in 7 patients affected with pseudoxanthoma elasticum, but failed to find evidence of disease-causing defects in any of these three genes. Five synonymous (G232G, P395P, A862A, G912G, D1106D), and five non synonymous (V4041, N458K, D490N, F1411, G1195R) polymorphisms were found in the pM5 gene,

Mutations in the gene encoding ABCR (ABCA4), a photoreceptor-specific ATP-binding cassette (ABC) transporter, are responsible for autosomal recessive Stargardt disease (STGD), an early onset macular degeneration, and some forms of autosomal recessive cone-rod dystrophy and autosomal recessive retinitis pigmentosa. Heterozygosity for ABCA4 mutations may also represent a risk factor for age-related macular degeneration (AMD). An ongoing challenge in the analysis of ABCA4-based retinopathies arises from the observation that most of the ABCA4 sequence variants identified so far are missense mutations that are rare in both patient and control populations. With the current sample size of most sequence variants, one cannot determine statistically whether a particular sequence variant is pathogenic or neutral. A related challenge is to determine the degree to which each pathogenic variant impairs ABCR function, as genotype-phenotype analyses indicate that age of onset and disease severity correlate with different ABCA4 alleles. To address these questions, a functional analysis of human ABCR and its variants was performed. These experiments reveal a wide spectrum of biochemical defects in these variants and provide insight into the transport mechanism of ABCR. (See Sun et al., Nat Genet 2000 October;26(2):242-6).

The disclosed NOV14 nucleic acid of the invention encoding a ABC transporter-like protein includes the nucleic acid whose sequence is provided in Table 14A or a fragment thereof. The invention also includes a mutant or variant nucleic acid any of whose bases may be changed from the corresponding base shown in Table 14A while still encoding a protein that maintains its ABC transporter-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 32 percent of the bases may be so changed.

The disclosed NOV14 protein of the invention includes the ABC transporter-like protein whose sequence is provided in Table 14B. The invention also includes a mutant or variant protein any of whose residues may be changed from the corresponding residue shown in Table 14B while still encoding a protein that maintains its ABC transporter-like activities and physiological functions, or a functional fragment thereof. In the mutant or variant protein, up to about 44 percent of the residues may be so changed.

The invention further encompasses antibodies and antibody fragments, such as F_{ab} or (F_{ab})₂, that bind immunospecifically to any of the proteins of the invention.

The above defined information for this invention suggests that this ABC transporter-like protein (NOV14) may function as a member of a “ABC transporter family”. Therefore, the NOV14 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV14 nucleic acids and proteins of the invention are useful in potential therapeutic applications implicated in diseases including but not limited to various pathologies and disorders as indicated below. For example, a cDNA encoding the ABC transporter-like protein (NOV14) may be useful in gene therapy, and the ABC transporter-like protein (NOV14) may be useful when administered to a subject in need thereof. By way of nonlimiting example, the compositions of the present invention will have efficacy for treatment of patients suffering from cancer, trauma, regeneration (in vitro and in vivo), viral/bacterial/parasitic infections, psoriasis, actinic keratosis, tuberous sclerosis, acne, hair growth/loss, allopecia, pigmentation disorders, endocrine disorders, or other pathologies or conditions. The NOV14 nucleic acid encoding the ABC transporter-like protein of the invention, or fragments thereof, may further be useful in diagnostic applications, wherein the presence or amount of the nucleic acid or the protein are to be assessed.

NOV14 nucleic acids and polypeptides are further useful in the generation of antibodies that bind immuno-specifically to the novel NOV14 substances for use in therapeutic or diagnostic methods. These antibodies may be generated according to methods known in the art, using prediction from hydrophobicity charts, as described in the “Anti-NOVX Antibodies” section below. The disclosed NOV14 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV15

A disclosed NOV51 nucleic acid of 2540 nucleotides (also referred to as CG57652-01) encoding a diacylglycerol kinase alpha-like protein is shown in Table 1 SA. Putative untranslated regions upstream and/or downstream from the coding region, if any, are underlined, and the start and stop codons are in bold letters.

TABLE 15A
NOV nucleotide sequence.

| | |
|---|---|
| GGGGCGGTCGCAGCTGAAGCAGGCCTACCCTCTGAAGAGGTCCAAGCAACGGAAGTACTA | (SEQ ID NO:39) |
| CTACGAAGCTGCCTTTCTGGCCATCCTTGAGAAAAATAGACAGATGGCCAAGGAGAGGGG | |
| CCTAATAAGCCCCAGTGATTTTGCCCAGCTGCAAAAATACATGGAATACTCCACCAAAAA | |
| GGTCAGTGATGTCCTAAAGCTCTTCGAGGATGGCGAGATGGCTAAATATGTCCAAGGAGA | |
| TGCCATTGGGTACGAGGGATTCCAGCAATTCCTGAAAATCTATCTCGAAGTGGATAATGT | |
| TCCCAGACACCTAAGCCTGGCACTGTTTCAATCCTTTGAGACTGGTCACTGCTTAAATGA | |
| GACAAATGTGACAAAAGATGTGGTGTGTCTCAATGATGTTTCCTGCTACTTTTCCCTTCT | |
| GGAGGGTGGTCGGCCAGAAGACAAGTTAGAATTCACCTTCAAGCTGTACGACACGGACAG | |
| AAATGGGATCCTGGACAGCTCAATGATGCGAGTGGCTGAATACCTGGATTGGGATGTGTC | |
| TGAGCTGAGGCCGATTCTTCAGGAGATGATGAAAGAGATTGACTATGATGGCAGTGGCTC | |
| TGTCTCTCAAGCTGAGTGGGTCCGGGCTGGGGCCACCACCGTGCCACTGCTAGTGCTGCT | |
| GGGTCTGGAGATGACTCTGAAGGACGACGGACAGCACATGTGGAGGCCCAAGAGGTTCCC | |
| CAGACCAGTCTACTGCAATCTGTGCGAGTCAAGCATTGGTCTTGGCAAACAGGGACTGAG | |
| CTGTAACCTCTGTAAGTACACTGTTCACGACCAGTGTGCCATGAAAGCCCTGCCTTGTGA | |
| AGTCAGCACCTATGCCAAGTCTCGGAAGGACATTGGTGTCCAATCACATGTGTGGGTGCG | |
| AGGAGGCTGTGAGTCCGGGCGCTGCGACCGCTGTCAGAAAAAGATCCGGATCTACCACAG | |
| TCTGACCGGGCTGCATTGTGTATGGTGCCACCTAGAGATCCACGATGACTGCCTGCAAGC | |
| GGTGGGCCATGAGTGTGACTGTGGGCTGCTCCGGGATCACATCCTGCCTCCATCTTCCAT | |
| CTATCCCAGTGTCCTGGCCTCTGGACCGGATCGTAAAAATAGCAAAACAAGCCAGAAGAC | |
| CATGGATGATTTAAATTTGAGCACCTCTGAGGCTCTGCGGATTGACCCTGTTCCTAACAC | |
| CCACCCACTTCTCGTCTTTGTCAATCCTAAGAGTGGCGGGAAGCAGGGGCAGAGGGTGCT | |
| CTGGAAGTTCCAGTATATATTAAACCCTCGACAGGTGTTCAACCTCCTAAAGGATGGTCC | |
| TGAGATAGGGCTCCGATTATTCAAGGATGTTCCTGATAGCCGGATTTTGGTGTGTGGTGG | |
| AGACGGCACAGTAGGCTGGATTCTAGAGACCATTGACAAAGCTAACTTGCCAGTTTTGCC | |
| TCCTGTTGCTGTGTTGCCCCTGGGTACTGGAAATGATCTGGCTCGATGCCTAAGATGGGG | |
| AGGAGGTTATGAAGGACAGAATCTGGCAAAGATCCTCAAGGATTTAGAGATGAGTAAAGT | |
| GGTACATATGGATCGATGGTCTGTGGAGGTGATACCTCAACAAACTGAAGAAAAAAGTGA | |
| CCCAGTCCCCTTTCAAATCATCAATAACTACTTCTCTATTGGCGTGGATGCCTCTATTGC | |
| TCATCGATTCCACATCATGCGAGAGAAATATCCGGAGAAGTTCAACAGCAGAATGAAGAA | |
| CAAGCTATGGTACTTCGAATTTGCCACATCTGAATCCATCTTCTCAACATGCAAAAAGCT | |
| GGAGGAGTCTTTGACAGTTGAGATCTGTGGGAAACCGCTGGATCTGAGCAACCTGTCCCT | |
| AGAAGGCATCGCAGTGCTAAACATCCCTAGCATGCATGGTGGCTCCAACCTCTGGGGTGA | |
| TACCAGGAGACCCCATGGGGATATCTATGGGATCAACCAGGCCTTAGGTGCTACAGCTAA | |
| AGTCATCACCGACCCTGATATCCTGAAAACCTGTGTACCAGACCTAAGTGACAAGAGACT | |
| GGAAGTGGTTGGGCTGGAGGGTGCAATTGAGATGGGCCAAATCTATACCAAGCTCAAGAA | |
| TGCTGGACGTCGGCTGGCCAAGTGCTCTGAGATCACCTTCCACACCACAAAAACCCTTCC | |
| CATGCAAATTGACGTAGAACCCTGGATGCAGACGCCCTGTACAATCAAGATCACCCACAA | |
| GAACCAGATGCCCATGCTCATGGGCCCACCCCCCCGCTCCACCAATTTCTTTGGCTTCTT | |
| GAGCTAAGGGGGACACCCTTGGCCTCCAAGCCAGCCTTGAACCCACCTCCCTGTCCCTGG | |
| ACTCTACTCCCGAGGCTCTGTACATTGCTGCCACATACTCCTGCCAGCTTGGGGGAGTGT | |
| TCCTTCACCCTCACAGTATTTATTATCCTGCACCACCTCACTGTTCCCCATGCGCACACA | |
| CATACACACACCCCAAAACACATACATTGAAAGTGCCTCATCTGAATAAAATGACTTGTG | |
| TTTCCCTTTGGGATCTGCTG | |

In a search of public sequence databases, the NOV15 nucleic acid sequence, located on chromsome 12 has 2038 of 2038 bases (100%) identical to a gb:GENBANK-ID:HSDKRNA|acc:X62535.1 mRNA from Homo sapiens (H.sapiens mRNA for diacylglycerol kinase). Public nucleotide databases include all GenBank databases and the GeneSeq patent database.

The disclosed NOV15 polypeptide (SEQ ID NO:40) encoded by SEQ ID NO:39 has 727 amino acid residues and is presented in Table 15B using the one-letter amino acid code. Signal P, Psort and/or Hydropathy results predict that NOV15 has no signal peptide and is likely to be localized in the nucleus with a certainty of 0.3000.

TABLE 15B
Encoded NOV15 protein sequence.

| | |
|---|---|
| MAKERGLISPSDFAQLQKYMEYSTKKVSDVLKLFEDGEMAKYVQGDAIGYEGFQQFLKIY | (SEQ ID NO:40) |
| LEVDNVPRHLSLALFQSFETGHCLNETNVTKDVVCLNDVSCYFSLLEGGRPEDKLEFTFK | |
| LYDTDRNGILDSSMMRVAEYLDWDVSELRPILQEMMKEIDYDGSGSVSQAEWVRAGATTV | |
| PLLVLLGLEMTLKDDGQHMWRPKRFPRPVYCNLCESSIGLGKQGLSCNLCKYTVHDQCAM | |
| KALPCEVSTYAKSRKDIGVQSHVWVRGGCESGRCDRCQKKIRIYHSLTGLHCVWCHLEIH | |
| DDCLQAVGHECDCGLLRDHILPPSSIYPSVLASGPDRKNSKTSQKTMDDLNLSTSEALRI | |
| DPVPNTHPLLVFVNPKSGGKQGQRVLWKFQYILNPRQVFNLLKDGPEIGLRLFKDVPDSR | |
| ILVCGGDGTVGWILETIDKANLPVLPPVAVLPLGTGNDLARCLRWGGGYEGQNLAKILKD | |
| LEMSKVVHMDRWSVEVIPQQTEEKSDPVPFQIINNYFSIGVDASIAHRFHIMREKYPEKF | |
| NSRMKNKLWYFEFATSESIFSTCKKLEESLTVEICGKPLDLSNLSLEGIAVLNIPSMHGG | |
| SNLWGDTRRPHGDIYGINQALGATAKVITDPDILKTCVPDLSDKRLEVVGLEGAIEMGQI | |
| YTKLKNAGRRLAKCSEITFHTTKTLPMQIDVEPWMQTPCTIKITHKNQMPMLMGPPPRST | |
| NFFGFLS | |

A search of sequence databases reveals that the NOV15 amino acid sequence has 727 of 735 amino acid residues (98%) identical to, and 727 of 735 amino acid residues (98%) similar to, the 735 amino acid residue ptnr:SWISSNEW-ACC:P23743 protein from Homo sapiens (Human) (DIACYLGLYCEROL KINASE, ALPHA (EC 2.7.1.107) (DIGLYCERIDE KINASE) (DGK-ALPHA) (DAG KFNASE ALPHA) (80 KDA DIACYLGLYCEROL KINASE)). Public amino acid databases include the GenBank databases, SwissProt, PDB and PIR.

NOV15 is expressed in at least Aorta, Appendix, Ascending Colon, Bone, Bone Marrow, Brain, Bronchus, Cartilage, Cervix, Colon, Coronary Artery, Dermis, Heart, Hippocampus, Kidney, Left cerebellum, Liver, Lung, Lymph node, Lymphoid tissue, Mammary gland/Breast, Ovary, Pancreas, Parotid Salivary glands, Peripheral Blood, Pituitary Gland, Placenta, Prostate, Respiratory Bronchiole, Small Intestine, Spleen, Stomach, Substantia Nigra, Synovium/Synovial membrane, Temporal Lobe, Testis, Thymus, Tonsils, Trachea, Umbilical Vein, Uterus, Vein, Whole Organism. Expression information was derived from the tissue sources of the sequences that were included in the derivation of the sequence of CG57652-01. The sequence is predicted to be expressed in the following tissues because of the expression pattern of (GEN BANK-ID: gb:GENBANK-ID:HSDKRNA|acc:X62535.1) a closely related H.sapiens mRNA for diacylglycerol kinase homolog in species Homo sapiens: lymphocytes, oligodendroglial cells, and neutrophils. This information was derived by determining the tissue sources of the sequences that were included in the invention including but not limited to SeqCalling sources, Public EST sources, Literature sources, and/or RACE sources.

The disclosed NOV15 polypeptide has homology to the amino acid sequences shown in the BLASTP data listed in Table 15C.

TABLE 15C
BLAST results for NOV15

| Gene Index/ | | Length | Identity | Positives | |
|---|---|---|---|---|---|
| Identifier | Protein/Organism | (aa) | (%) | (%) | Expect |
| gi\|11415024\|ref\|NP_ | diacylglycerol | 735 | 727/735 | 727/735 | 0.0 |
| 001336.1\| | kinase, alpha | | (98%) | (98%) | |
| (NM_001345) | (80 kD) [Homo | | | | |
| | sapiens] | | | | |
| gi\|125323\|sp\|P20192\| | Diacylglycerol | 734 | 679/734 | 703/734 | 0.0 |
| KDGA_PIG | kinase, alpha | | (92%) | (95%), | |
| | (Diglyceride | | | | |
| | kinase) (DGK- | | | | |
| | alpha) (DAG | | | | |
| | kinase alpha) (80 kDa | | | | |
| | diacylglycerol | | | | |
| | kinase) | | | | |
| gi\|13879470\|gb\|AAH06713.1\| | diacylglycerol | 730 | 597/736 | 647/736 | 0.0 |
| AAH06713 | kinase, alpha (80 kDa) | | (81%) | (87%), | |
| (BC006713) | [Mus | | | | |
| | musculus] | | | | |
| gi\|18158459\|ref\|NP_ | diacylglycerol | 727 | 600/736 | 646/736 | 0.0 |
| 542965.1\| | kinase, alpha | | (81%) | (87%), | |
| (NM_080787) | (80 kD) [Rattus | | | | |
| | norvegicus] | | | | |
| gi\|7949033\|ref\|NP_058091.1\| | diacylglycerol | 730 | 594/736 | 645/736 | 0.0 |
| (NM_016811) | kinase, alpha (80 kDa) | | (80%) | (86%) | |
| | [Mus | | | | |
| | musculus] | | | | |

Tables 15D-E list the domain descriptions from DOMAIN analysis results against NOV15. This indicates that the NOV15 sequence has properties similar to those of other proteins known to contain this domain.

TABLE 15D
Domain Analysis of NOV15

gnl|Pfam|pfam00609, DAGKa, Diacylglycerol kinase accessory domain
(presumed). Diacylglycerol (DAG) is a second messenger that acts as a
protein kinase C activator
CD-Length = 170 residues, 100.0% aligned
Score = 275 bits (702), Expect = 9e−75

TABLE 15E
Domain Analysis of NOV15

gnl|Smart|smart00109, C1, Protein kinase C conserved region 1 (C1)
domains (Cysteine-rich domains); Some bind phorbol esters and
diacylglycerol. Some bind RasGTP. Zinc-binding domains.
CD-Length = 50 residues, 96.0% aligned
Score = 53.5 bits (127), Expect = 4e−08

Diacylglycerol (DAG) functions in intracellular signaling pathways as an allosteric activator of protein kinase C (PKC; see 600448). In addition, DAG appears to play a role in regulating RAS (see 190020) and RHO (see 165370) family proteins by activating the guanine nucleotide exchange factors VAV (164875) and RASGRP1 (603962). DAG also occupies a central position in the synthesis of major phospholipids and triacylglycerols. Thus, to maintain cellular homeostasis, intracellular DAG levels must be tightly regulated (Topham and Prescott, 1999). DAG kinases (DGKs or DAGKs; EC 2.7.1.107) phosphorylate DAG to phosphatidic acid, thus removing DAG. In intracellular signaling pathways, DAGK can be viewed as a modulator that competes with PKC for the second messenger DAG. Schaap et al. (1990) purified and characterized an 86-kD DAGK from normal human white blood cells. Based on partial amino acid sequences of the purified enzyme, primers were designed that permitted cloning of the human DAGK cDNA by use of PCR. The sequence demonstrated that it is the human homolog of the porcine gene. The human DAGK cDNA, transfected into COS-7 cells, resulted in a 6- to 7-fold increase in enzyme activity.

Several mammalian isozymes of DAGK have been identified. The isoform described by Schaap et al. (1990) has been designated DGK-alpha or DAGK1. Topham and Prescott (1999) stated that all DGKs have a conserved catalytic domain and at least 2 cysteine-rich regions homologous to the C1A and C1B motifs of PKCs. Most DGKs have structural motifs that are likely to play regulatory roles, and these motifs form the basis for dividing the DGKs into 5 subtypes. Type I DGKs, such as DGK-alpha, -beta (604070), and -gamma (601854), have calcium-binding EF-hand motifs at their N termini. DGK-delta (601826) and DKG-eta (604071) contain N-terminal pleckstrin homology (PH) domains and are defined as type II. DGK-epsilon (601440) contains no identifiable regulatory domains and is a type III DGK. The defining characteristic of type IV isozymes, such as DGK-zeta (601441) and -iota (604072), is that they have C-terminal ankyrin repeats. Group V is exemplified by DGK-theta (601207), which contains 3 cysteine-rich domains and a PH domain.

Pilz et al. (1995) pointed to the growing evidence to support some form of light-activated phosphoinositide signal transduction pathway in the mammalian retina. Although this pathway had no obvious role in mammalian phototransduction, mutations in this pathway were known to cause retinal degeneration in Drosophila. For example, the ‘retinal degeneration A’ mutant in Drosophila is caused by an alteration in the eye-specific DAGK gene. In an effort to consider genes mutated in Drosophila as candidates for mammalian eye disease, Pilz et al. (1995) determined the map position of 3 DAGK genes in the mouse. They localized the mouse homolog of DAGK1 to chromosome 10 by linkage analysis. By Southern blot analysis of human-hamster somatic cell hybrid DNA, Hart et al. (1994) assigned the DAGK gene to chromosome 12. Hart et al. (1994) further localized the gene to 12q13.3 by fluorescence in situ hybridization.

The disclosed NOV15 nucleic acid of the invention encoding a diacylglycerol kinase alpha-like protein includes the nucleic acid whose sequence is provided in Table 15A or a fragment thereof. The invention also includes a mutant or variant nucleic acid any of whose bases may be changed from the corresponding base shown in Table 15A while still encoding a protein that maintains its diacylglycerol kinase alpha-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 0 percent of the bases may be so changed.

The disclosed NOV15 protein of the invention includes the diacylglycerol kinase alpha-like protein whose sequence is provided in Table 15B. The invention also includes a mutant or variant protein any of whose residues may be changed from the corresponding residue shown in Table 1 SB while still encoding a protein that maintains its diacylglycerol kinase alpha-like activities and physiological functions, or a functional fragment thereof. In the mutant or variant protein, up to about 2 percent of the residues may be so changed.

The invention further encompasses antibodies and antibody fragments, such as F_{ab} or (F_{ab})₂, that bind immunospecifically to any of the proteins of the invention.

The above defined information for this invention suggests that this diacylglycerol kinase alpha-like protein (NOV15) may function as a member of a “diacylglycerol kinase alpha family”. Therefore, the NOV15 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV15 nucleic acids and proteins of the invention are useful in potential therapeutic applications implicated in diseases including but not limited to various pathologies and disorders as indicated below. For example, a cDNA encoding the diacylglycerol kinase alpha-like protein (NOV15) may be useful in gene therapy, and the diacylglycerol kinase alpha-like protein (NOV15) may be useful when administered to a subject in need thereof. By way of nonlimiting example, the compositions of the present invention will have efficacy for treatment of patients suffering from osteoporosis, hypercalceimia, arthritis, ankylosing spondylitis, scoliosis, hemophilia, hypercoagulation, idiopathic thrombocytopenic purpura, autoimmune disease, allergies, asthma, immunodeficiencies, transplantation, graft versus host disease, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, tendonitis, fertility, atherosclerosis, aneurysm, hypertension, fibromuscular dysplasia, scleroderma, myocardial infarction, embolism, cardiovascular disorders, bypass surgery, cardiomyopathy, atherosclerosis, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, renal artery stenosis, interstitial nephritis, glomerulonephritis, polycystic kidney disease, renal tubular acidosis, IgA nephropathy, cirrhosis, systemic lupus erythematosus, emphysema, ARDS, lymphedema, endometriosis, diabetes, pancreatitis, obesity, anemia, ataxia-telangiectasia, endocrine dysfunctions, growth and reproductive disorders, inflammatory bowel disease, diverticular disease, ulcers, tonsillitis, ARDS, anemia, bleeding disorders, or other pathologies or conditions. The NOV15 nucleic acid encoding the diacylglycerol kinase alpha-like protein of the invention, or fragments thereof, may further be useful in diagnostic applications, wherein the presence or amount of the nucleic acid or the protein are to be assessed.

NOV15 nucleic acids and polypeptides are further useful in the generation of antibodies that bind immuno-specifically to the novel NOV15 substances for use in therapeutic or diagnostic methods. These antibodies may be generated according to methods known in the art, using prediction from hydrophobicity charts, as described in the “Anti-NOVX Antibodies” section below. The disclosed NOV15 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV16

A disclosed NOV16 nucleic acid of 3904 nucleotides (also referred to as CG57562-01) encoding a cation-transporting ATPase-like protein is shown in Table 16A. Putative untranslated regions upstream and/or downstream from the coding region, if any, are underlined, and the start and stop codons are in bold letters.

TABLE 16A
NOV16 nucleotide sequence.

| | |
|---|---|
| TTACCGGAAGTAAAACTTCGGAAGTGAGGCGTTCCTCTGCCCGGAAGTGAGCGCGGCGCT | (SEQ ID NO:41) |
| AGGAAAGATGGCGGCAGCGGCGGCGGTGGGCAACGCGGTGCCCTGCGGGGCCCGGCCTTG | |
| CGGGGTCCGGCCTGACGGGCAGCCCAAGCCCGGGCGCAGCCGGCGCGCGCTCCTTGCCGC | |
| CGGGCCGGCGCTCATAGCGAACGGTGACGAGCTGGTGGCTGCCGTGTGGCCGTACCGGCG | |
| GTTGGCGCTGTTGCGGCGCCTCACGGTGCTGCCATTCGCCGGGCTGCTTTACCCGGCCTG | |
| GTTGGGTGCCGCAGCCGCTGGCTGCTGGGGCTGGGGCAGCAGTTGGGTGCAGATCCCCGA | |
| AGCTGCGCTGCTCGTGCTTGCCACCATCTGCCTCGCGCACGCGCTCACTGTCCTCTCGGG | |
| GCATTGGTCTGTGCACGCGCATTGCGCGCTCACCTGCACCCCGGAGTACGACCCCAGCAA | |
| AGCGACCTTTGTGAAGGTGGTGCCAACCCCCAACAATGGCTCCACGGAGCTCGTGGCCCT | |
| GCACCGCAATGAGGGCGAAGACGGGCTTGAGGTGCTGTCCTTCGAATTCCAGAAGATCAA | |
| GTATTCCTACGATGCCCTGGAGAAGAAGCAGTTTCTCCCCGTGGCCTTTCCTGTGGGAAA | |
| CGCCTTCTCATACTATCAGAGCAACAGAGGCTTCCAGGAAGACTCAGAGATCCGAGCAGC | |
| TGAGAAGAAATTTGGGAGCAACAAGGCCGAGATGGTGGTGCCTGACTTCTCGGAGCTTTT | |
| CAAGGAGAGAGCCACAGCCCCCTTCTTTGTATTTCAGGTGTTCTGTGTGGGGCTCTGGTG | |
| CCTGGATGAGTACTGGTACTACAGCGTCTTTACGCTATCCATGCTGGTGGCGTTCGAGGC | |
| CTCGCTGGTGCAGCAGCAGATGCGGAACATGTCGGAGATCCGGAAGATGGGCAACAAGCC | |
| CCACATGATCCAGGTCTACCGAAGCCGCAAGTGGAGGCCCATTGCCAGTGATGAGATCGT | |
| ACCAGGGGACATCGTCTCCATCGGCCGCTCCCCACAGGAGAACCTGGTGCCATGTGACGT | |
| GCTTCTGCTGCGAGGCCGCTGCATCGTAGACGAGGCCATGCTCACGGGGGAGTCCGTGCC | |
| ACAGATGAAGGAGCCCATCGAAGACCTCAGCCCAGACCGGGTGCTGGACCTCCAGGCTGA | |
| TTCCCGGCTGCACGTCATCTTCGGGGGCACCAAGGTGGTGCAGCACATCCCCCCACAGAA | |
| AGCCACCACGGGCCTGAAGCCGGTTGACAGCGGGTGCGTGGCCTACGTCCTGCGGACCGG | |
| ATTCAACACATCCCAGGGCAAGCTGCTGCGCACCATCCTCTTCGGGGTCAAGAGGGTGAC | |
| TGCGAACAACCTGGAGACCTTCATCTTCATCCTCTTCCTCCTGGTGTTTGCCATCGCTGC | |
| AGCTGCCTATGTATGGATTGAAGGTACCAAGGACCCCAGCCGGAACCGCTACAAGCTGTT | |
| TCTGGAGTGCACCCTGATCCTCACCTCGGTCGTGCCTCCTGAGCTGCCCATCGAGCTGTC | |
| CCTGGCCGTCAACACCTCCCTCATCGCCCTGGCCAAGCTCTACATGTACTGCACAGAGCC | |
| CTTCCGGATCCCCTTTGCTGGCAAGGTCGAGGTGTGCTGCTTTGACAAGACGGGGACGTT | |
| GACCAGTGACAGCCTGGTGGTGCGCGGTGTGGCCGGGCTGAGAGACGGGAAGGAGGTGAC | |
| CCCAGTGTCCAGCATCCCTGTAGAAACACACCGGGCCCTGGCCTCGTGCCACTCGCTCAT | |
| GCAGCTGGACGACGGCACCCTCGTGGGTGACCCTCTAGAGAAGGCCATGCTGACGGCCGT | |
| GGACTGGACGCTGACCAAAGATGAGAAAGTATTCCCCCGAAGTATTAAAACTCAGGGGCT | |
| GAAAATTCACCAGCGCTTTCATTTTGCCAGTGCCCTGAAGCGAATGTCCGTGCTTGCCTC | |
| GTATGAGAAGCTGGGCTCCACCGACCTCTGCTACATCGCGGCCGTGAAGGGGGCCCCCGA | |
| AACTCTGCACTCCATGTTCTCCCAGTGCCCGCCCGACTACCACCACATCCACACCGAGAT | |
| CTCCCGGGAAGGAGCCCGCGTCCTGGCGCTGGGGTACAAGGAGCTGGGACACCTCACTCA | |
| CCAGCAGGCCCGGGAGGTCAAGCGGGAGGCCCTGGAGTGCAGCCTCAAGTTCGTCGGCTT | |
| CATTGTGGTCTCCTGCCCGCTCAAGGCTGACTCCAAGGCCGTGATCCGGGAGATCCAGAA | |
| TGCGTCCCACCGGGTGGTCATGATCACGGGAGACAACCCGCTCACTGCATGCCACGTGGC | |
| CCAGGAGCTGCACTTCATTGAAAAGGCCCACACGCTGATCCTGCAGCCTCCCTCCGAGAA | |
| AGGCCGGCAGTGCGAGTGGCGCTCCATTGACGGCAGCATCGTGCTGCCCCTGGCCCGGGG | |
| CTCCCCAAAGGCACTGGCCCTGGAGTACGCACTGTGCCTCACAGGCGACGGCTTGGCCCA | |
| CCTGCAGGCCACCGACCCCCAGCAGCTGCTCCGCCTCATCCCCCATGTGCAGGTGTTCGC | |
| CCGTGTGGCTCCCAAGCAGAAGGAGTTTGTCATCACCAGCCTGAAGGAGCTGGGCTACGT | |
| GACCCTCATGTGTGGGGATGGCACCAACGACGTGGGCGCCCTGAAGCATGCTGACGTGGG | |
| TGTGGCGCTCTTGGCCAATGCCCCTGAGCGGGTTGTCGAGCGGCGACGGCGGCCCCGGGA | |
| CAGCCCAACCCTGAGCAACAGTGGCATCAGAGCCACCTCCAGGACAGCCAAGCAGCGGTC | |
| GGGGCTCCCTCCCTCCGAGGAGCAGCCAACCTCCCAGAGGGACCGCCTGAGCCAGGTGCT | |
| GCGAGACCTCGAGGACGAGAGTACGCCCATTGTGAAACTGGGGGATGCCAGCATCGCAGC | |
| ACCCTTCACCTCCAAGCTCTCATCCATCCAGTGCATCTGCCACGTGATCAAGCAGGGCCG | |
| CTGCACGCTGGTGACCACGCTACAGATGTTCAAGATCCTGGCGCTCAATGCCCTCATCCT | |
| GGCCTACAGCCAGAGCGTCCTCTACCTGGAGGGAGTCAAGTTCAGTGACTTCCAGGCCAC | |
| CCTACAGGGGCTGCTGCTGGCCGGCTGCTTCCTCTTCATCTCCCGTTCCAAGCCCCTCAA | |
| GACCCTCTCCCGAGAACGGCCCCTGCCCAACATCTTCAACCTGTACACCATCCTCACCGT | |
| CATGCTCCAGTTCTTTGTGCACTTCCTGAGCCTTGTCTACCTGTACCGTGAGGCCCAGGC | |
| CCGGAGCCCCGAGAAGCAGGAGCAGTTCGTGGACTTGTACAAGGAGTTTGAGCCAAGCCT | |
| GGTCAACAGCACCGTCTACATCATGGCCATGGCCATGCAGATGGCCACCTTCGCCATCAA | |
| TTACAAAGGCCCGCCCTTCATGGAGAGCCTGCCCGAGAACAAGCCCCTGGTGTGGAGTCT | |
| GGCAGTTTCACTCCTGGCCATCATTGGCCTGCTCCTCGGCTCCTCGCCCGACTTCAACAG | |
| CCAGTTTGGCCTCGTGGACATCCCTGTGGAGTTCAAGCTGGTCATTGCCCAGGTCCTGCT | |
| CCTGGACTTCTGCCTGGCGCTCCTGGCCGACCGCGTCCTGCAGTTCTTCCTGGGGACCCC | |
| GAAGCTGAAAGTGCCTTCCTGAGATGGCAGTGCTGGTACCCACTGCCCACCCTGGCTGCC | |
| GCTGGGCGGGAACCCCAACAGGGCCCCGGGAGGGAACCCTGCCCCCAACCCCCCACAGCA | |
| AGGCTGTACAGTCTCGCCCTTGGAAGACTGAGCTGGGACCCCCACAGCCATCCGCTGGCT | |
| TGGCCAGCAGAACCAGCCCCAAGCCAGCACCTTTGGTAAATAAAGCAGCATCTGAGATTT | |
| TAAA | |

In a search of public sequence databases, the NOV16 nucleic acid sequence, located on chromsome 19 has 3442 of 3442 bases (100%) identical to a gb:GENBANK-ID:AF288687|acc:AF288687.1 mRNA from Homo sapiens (Homo sapiens CGI-152 protein mRNA, complete cds). Public nucleotide databases include all GenBank databases and the GeneSeq patent database.

The disclosed NOV16 polypeptide (SEQ ID NO:42) encoded by SEQ ID NO:41 has 1204 amino acid residues and is presented in Table 16B using the one-letter amino acid code. Signal P, Psort and/or Hydropathy results predict that NOV16 has a signal peptide and is likely to be localized at the plasma membrane with a certainty of 0.8000.

TABLE 16B
Encoded NOV16 protein sequence.

| | |
|---|---|
| MAAAAAVGNAVPCGARPCGVRPDGQPKPGRSRRALLAAGPALIANGDELVAAVWPYRRLA | (SEQ ID NO:42) |
| LLRRLTVLPFAGLLYPAWLGAAAAGCWGWGSSWVQIPEAALLVLATICLAHALTVLSGHW | |
| SVHAHCALTCTPEYDPSKATFVKVVPTPNNGSTELVALHRNEGEDGLEVLSFEFQKIKYS | |
| YDALEKKQFLPVAFPVGNAFSYYQSNRGFQEDSEIRAAEKKFGSNKAEMVVPDFSELFKE | |
| RATAPFFVFQVFCVGLWCLDEYWYYSVFTLSMLVAFEASLVQQQMRNMSEIRKMGNKPHM | |
| IQVYRSRKWRPIASDEIVPGDIVSIGRSPQENLVPCDVLLLRGRCIVDEAMLTGESVPQM | |
| KEPIEDLSPDRVLDLQADSRLHVIFGGTKVVQHIPPQKATTGLKPVDSGCVAYVLRTGFN | |
| TSQGKLLRTILFGVKRVTANNLETFIFILFLLVFAIAAAAYVWIEGTKDPSRNRYKLFLE | |
| CTLILTSVVPPELPIELSLAVNTSLIALAKLYMYCTEPFRIPFAGKVEVCCFDKTGTLTS | |
| DSLVVRGVAGLRDGKEVTPVSSIPVETHRALASCHSLMQLDDGTLVGDPLEKAMLTAVDW | |
| TLTKDEKVFPRSIKTQGLKIHQRFHFASALKRMSVLASYEKLGSTDLCYIAAVKGAPETL | |
| HSMFSQCPPDYHHIHTEISREGARVLALGYKELGHLTHQQAREVKREALECSLKFVGFIV | |
| VSCPLKADSKAVIREIQNASHRVVMITGDNPLTACHVAQELHFIEKAHTLILQPPSEKGR | |
| QCEWRSIDGSIVLPLARGSPKALALEYALCLTGDGLAHLQATDPQQLLRLIPHVQVFARV | |
| APKQKEFVITSLKELGYVTLMCGDGTNDVGALKHADVGVALLANAPERVVERRRRPRDSP | |
| TLSNSGIRATSRTAKQRSGLPPSEEQPTSQRDRLSQVLRDLEDESTPIVKLGDASIAAPF | |
| TSKLSSIQCICHVIKQGRCTLVTTLQMFKILALNALILAYSQSVLYLEGVKFSDFQATLQ | |
| GLLLAGCFLFISRSKPLKTLSRERPLPNIFNLYTILTVMLQFFVHFLSLVYLYREAQARS | |
| PEKQEQFVDLYKEFEPSLVNSTVYIMAMAMQMATFAINYKGPPFMESLPENKPLVWSLAV | |
| SLLAIIGLLLGSSPDFNSQFGLVDIPVEFKLVIAQVLLLDFCLALLADRVLQFFLGTPKL | |
| KVPS | |

A search of sequence databases reveals that the NOV16 amino acid sequence has 1141 of 1200 amino acid residues (95%) identical to, and 1164 of 1200 amino acid residues (97%) similar to, the 1200 amino acid residue ptnr:TREMBLNEW-ACC:BAB20095 protein from Mus musculus (Mouse) (CATION-TRANSPORTING ATPASE). Public amino acid databases include the GenBank databases, SwissProt, PDB and PIR.

NOV16 is expressed in at least Adrenal Gland/Suprarenal gland, Amygdala, Aorta, Appendix, Artery, Bone, Bone Marrow, Brain, Bronchus, Brown adipose, Cartilage, Cerebral Medulla/Cerebral white matter, Cervix, Colon, Coronary Artery, Epidermis, Hair Follicles, Heart, Hippocampus, Kidney, Left cerebellum, Liver, Lung, Lymph node, Lymphoid tissue, Mammary gland/Breast, Ovary, Oviduct/Uterine Tube/Fallopian tube, Pancreas, Parietal Lobe, Peripheral Blood, Pituitary Gland, Placenta, Prostate, Respiratory Bronchiole, Right Cerebellum, Skeletal Muscle, Skin, Spinal Cord, Spleen, Stomach, Substantia Nigra, Synovium/Synovial membrane, Temporal Lobe, Testis, Thymus, Urinary Bladder, Uterus, Vein, and Vulva. This information was derived by determining the tissue sources of the sequences that were included in the invention including but not limited to SeqCalling sources, Public EST sources, Literature sources, and/or RACE sources.

The disclosed NOV16 polypeptide has homology to the amino acid sequences shown in the BLASTP data listed in Table 16C.

TABLE 16C
BLAST results for NOV16

| Gene Index/ | | Length | Identity | Positives | |
|---|---|---|---|---|---|
| Identifier | Protein/Organism | (aa) | (%) | (%) | Expect |
| gi\|14017867\|dbj\|BAB47454.1\| | KIAA1825 protein | 1203 | 1200/1203 | 1200/1203 | 0.0 |
| (AB058728) | [Homo sapiens] | | (99%) | (99%) | |
| gi\|18202961\|sp\|Q9HD20\| | Probable cation- | 1204 | 1201/1204 | 1201/1204 | 0.0 |
| ATY2_HUMAN | transporting | | (99%) | (99%) | |
| | ATPase 2 (CGI- | | | | |
| | 152) | | | | |
| gi\|18202867\|sp\|Q9EPE9\| | Probable cation- | 1200 | 1141/1200 | 1164/1200 | 0.0 |
| ATY2_MOUSE | transporting | | (95%) | (96%), | |
| | ATPase 2 (CATP) | | | | |
| gi\|9966897\|ref\|NP_065143.1\| | CGI-152 protein | 1086 | 1072/1072 | 1072/1072 | 0.0 |
| (NM_020410) | [Homo sapiens] | | (100%) | (100%) | |
| gi\|18467838\|ref\|XP_ | BcDNA: GH06032 | 1225 | 611/1125 | 803/1125 | 0.0 |
| 079402.1\| | [Drosophila | | (54%) | (71%), | |
| (XM_079402) | melanogaster] | | | | |

Tables 16D-E list the domain descriptions from DOMAIN analysis results against NOV16. This indicates that the NOV16 sequence has properties similar to those of other proteins known to contain this domain.

TABLE 16D
Domain Analysis of NOV16

gnl|Pfam|pfam00122, E1-E2_ATPase, E1-E2 ATPase
CD-Length = 223 residues, 57.4% aligned
Score = 64.3 bits (155), Expect = 4e−11

TABLE 16E
Domain Analysis of NOV16

gnl|Pfam|pfam00702, Hydrolase, haloacid dehalogenase-like hydrolase.
This family are structurally different from the alpha/beta hydrolase
family (pfam00561). This family includes L-2-haloacid dehalogenase,
epoxide hydrolases and phosphatases. The structure of the family
consists of two domains. One is an inserted four helix bundle, which
is the least well conserved region of the alignment, between residues
16 and 96. The rest of the fold is composed of the core alpha/beta
domain.
CD-Length = 197 residues, 78.2% aligned
Score = 39.7 bits (91), Expect = 0.001

Regulation of cation homeostasis is of critical importance to the cell and defects in proteins that regulate this process have been shown to cause a number of human diseases, including Darier-White disease, Menkes disease and Wilson disease. The human plasma membrane Ca(2+)-ATPase (PMCA) isoforms are members of the P class of ion-motive ATPases. PMCA removes bivalent calcium ions from eukaryotic cells and plays a critical role in intracellular calcium homeostasis by its capacity for removing calcium ions from cells against very large concentration gradients. Together with the highly related SERCA1 and SERCA3 isoforms encoded by ATP2A1 and ATP2A3, respectively, SERCA2 belongs to the large family of P-type cation pumps that couple ATP hydrolysis with cation transport across membranes. SERCA pumps specifically maintain low cytosolic Ca(2+) concentrations by actively transporting Ca(2+) from the cytosol into the sarco/endoplasmic reticulum lumen. The ATP2A2 gene has been shown to be the site of mutations in Darier-White disease, an autosomal dominant skin disorder characterized by warty papules and plaques in seborrheic areas (central trunk, flexures, scalp, and forehead), palmoplantar pits, and distinctive nail abnormalities (See Sakuntabhai et al., (1999) Mutations in ATP2A2, encoding a Ca(2+) pump, cause Darier disease. Nature Genet. 21: 271-277). Patients with Menkes disease have mutations in the gene encoding Cu(2+)-transporting ATPase alpha polypeptide and display early retardation in growth, peculiar hair, and focal cerebral and cerebellar degeneration ((See Chelly et al., (1993) Isolation of a candidate gene for Menkes disease that encodes a potential heavy metal binding protein. Nature Genet. 3: 14-19). Wilson disease is an autosomal recessive disorder caused by mutations in the ATP7B gene, which encodes a copper-transporting ATPase, and is characterized by dramatic build-up of intracellular hepatic copper with subsequent hepatic and neurologic abnormalities (See Bull et al. (1993) The Wilson disease gene is a putative copper transporting P-type ATPase similar to the Menkes gene. Nature Genet. 5: 327-337).

The human cation transporting ATPase-like protein described in this invention is predicted to share the attributes of the other cation transporting ATPase family members and is thus implicated in the regulation of cation homeostasis. Given that a large number of cation transporting ATPases have been demonstrated to have a causative role in a variety of human diseases, the cation transporting ATPase-like protein is an attractive target for drug intervention in the treatment of human metabolic diseases, central nervous system disorders, immunological diseases and cancer, among others. The cation transporting ATPase-like gene described in this patent maps to human chromosome 19.

The disclosed NOV16 nucleic acid of the invention encoding a cation-transporting ATPase-like protein includes the nucleic acid whose sequence is provided in Table 16A or a fragment thereof. The invention also includes a mutant or variant nucleic acid any of whose bases may be changed from the corresponding base shown in Table 16A while still encoding a protein that maintains its cation-transporting ATPase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 0 percent of the bases may be so changed.

The disclosed NOV16 protein of the invention includes the cation-transporting ATPase-like protein whose sequence is provided in Table 16B. The invention also includes a mutant or variant protein any of whose residues may be changed from the corresponding residue shown in Table 16B while still encoding a protein that maintains its cation-transporting ATPase-like activities and physiological functions, or a functional fragment thereof. In the mutant or variant protein, up to about 5 percent of the residues may be so changed.

The invention further encompasses antibodies and antibody fragments, such as F_{ab} or (F_{ab})₂, that bind immunospecifically to any of the proteins of the invention.

The above defined information for this invention suggests that this cation-transporting ATPase-like protein (NOV16) may function as a member of a “cation-transporting ATPase family”. Therefore, the NOV16 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV16 nucleic acids and proteins of the invention are useful in potential therapeutic applications implicated in diseases including but not limited to various pathologies and disorders as indicated below. For example, a cDNA encoding the cation-transporting ATPase-like protein (NOV16) may be useful in gene therapy, and the cation-transporting ATPase-like protein (NOV16) may be useful when administered to a subject in need thereof. By way of nonlimiting example, the compositions of the present invention will have efficacy for treatment of patients suffering from cancer, trauma, bacterial and viral infections, in vitro and in vivo regeneration, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, aneurysm, hypertension, fibromuscular dysplasia, stroke, obesity, transplantation, myocardial infarction, embolism, cardiovascular disorders, bypass surgery, anemia, bleeding disorders, adrenoleukodystrophy, congenital adrenal hyperplasia, diabetes, Von Hippel-Lindau (VHL) syndrome, pancreatitis, fertility, endometriosis, hypogonadism, hypercalceimia, ulcers, cirrhosis, Hirschsprung's disease, Crohn's Disease, appendicitis, hemophilia, hypercoagulation, idiopathic thrombocytopenic purpura, ataxia-telangiectasia, lymphedema, allergies, hemophilia, autoimmune disease, allergies, immunodeficiencies, transplantation, graft versus host disease (GVHD), osteoporosis, arthritis, ankylosing spondylitis, scoliosis, arthritis, tendonitis, muscular dystrophy, Lesch-Nyhan syndrome, myasthenia gravis, Alzheimer's disease, Parkinson's disease, Huntington's disease, cerebral palsy, epilepsy, multiple sclerosis, leukodystrophies, behavioral disorders, addiction, anxiety, pain, neurodegeneration, endocrine dysfunctions, growth and reproductive disorders, systemic lupus erythematosus, asthma, emphysema, ARDS, psoriasis, actinic keratosis, acne, hair growth/loss, allopecia, pigmentation disorders, cystitis, incontinence, renal artery stenosis, interstitial nephritis, glomerulonephritis, polycystic kidney disease, renal tubular acidosis, and IgA nephropathy, or other pathologies or conditions. The NOV16 nucleic acid encoding the cation-transporting ATPase-like protein of the invention, or fragments thereof, may further be useful in diagnostic applications, wherein the presence or amount of the nucleic acid or the protein are to be assessed.

NOV16 nucleic acids and polypeptides are further useful in the generation of antibodies that bind immuno-specifically to the novel NOV16 substances for use in therapeutic or diagnostic methods. These antibodies may be generated according to methods known in the art, using prediction from hydrophobicity charts, as described in the “Anti-NOVX Antibodies” section below. The disclosed NOV16 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV17

A disclosed NOV17 nucleic acid of 1167 nucleotides (also referred to as CG55914-01) encoding an acyl CoA desaturase-like protein is shown in Table 17A. Putative untranslated regions upstream and/or downstream from the coding region, if any, are underlined, and the start and stop codons are in bold letters.

TABLE 17A
NOV17 nucleotide sequence.

| | |
|---|---|
| ATGGGGCAGTTTTATAGGACTGGGGCAGGCAGTGGAAAGTTACAGTTAAAGGTGGTTATC | (SEQ ID NO:43) |
| TATTGTCAGCTGAGGAGGGATCACAAGGTGAATGGTGAGGAGATCATAAGACTCATTGTC | |
| CAAAAGAGGAATGTCACGAGGTCAATCGATCGATCAGTTGGGGCAGGGCAGGAACAAGTC | |
| ATAATGGAATGTCTTAAGCCTTTTTACTTCAACTATCCATCTCTAGACAGTGAGGTCCTG | |
| GATGATGACAGAGCCATAGATGGAAAAGACACCATTATTCTGGTCTATAAAGAACTGTCT | |
| AGGGACTTGGCATCCTGTGTCCCAGCCACTCCAGCTGTGGCTGAAAGGGTCCAAGGTACA | |
| GTTCAGGCCATGGCTTCAAAGGGTGCAAGCCCCAAGTCTCGGCAGCTTTCACAAGGTGTT | |
| AAGCCTGGCAGTACAGAAGGGAAATGTGGGTTGGAGCCCCAACACAGAGCCCCACTGGGG | |
| ACACTGCCTAGTGGAGCTTTGAGAAGAGGGCCACCATTCTCCAGACCCCAGAATGGTAGA | |
| CCCACTGACAGCTTGCACTATGCACTTGGAAAAGACACAGACACTCAACACCAGCCCATG | |
| AAAGCAGCCAGAAGGGAGGCTGTACCCTGCACAGCTACAGGGGCAGAGCTGCCCAAGACC | |
| ATGGGAACCCAACTGTTGCATCAGCATGACCCAGATGTGAGAATTGGAGTCAAAGAAGAT | |
| CATTTTGGAGCTTTAAGATTTGACTGTCCTTCTAGATTTTGGACTTACATGAGGACCCCA | |
| GCCTTGCTGCTCTGCCCATTGACCTCTGCCACCCTCCATACCGGGTGTGAGTTACCACCA | |
| GAAGAAGTCTGTGGCAGCAGCAGTCTTTGCCTTATTCTGACAGTGCCAAAGTGTGTTTGG | |
| CCCTACCAACAACTGCAAGCCCTTTCCTTGCTTTACTTTAGCTTTCCCTCCAGAAGGCTC | |
| ATTGCTTTGAAATGTAACATATCCTGGTCCAACTACATCCGATTCCATGGGTCTGCATCA | |
| TTATCTCCAAAACCTTCCAGTTGCATTGCTGTCATGTTTGTGTTAAAGAGACTGATGAGT | |
| CTGGACTATGACCGGAAGAAAGTCTCCACGGCCGCCATCTTGGCCAGGATTAAAAGAACT | |
| GGAGATGGAAGCTACAAGAGTGGCTGA | |

In a search of public sequence databases, the NOV17 nucleic acid sequence, located on chromsome 5 has 316 of 381 bases (82%) identical to a gb:GENBANK-ID:AK000899|acc:AK000899.1 mRNA from Homo sapiens (Homo sapiens cDNA FLJ10037 fis, clone HEMBA1000968). Public nucleotide databases include all GenBank databases and the GeneSeq patent database.

The disclosed NOV17 polypeptide (SEQ ID NO:44) encoded by SEQ ID NO:43 has 388 amino acid residues and is presented in Table B using the one-letter amino acid code. Signal P, Psort and/or Hydropathy results predict that NOV17 has no signal peptide and is likely to be localized in the cytoplasm with a certainty of 0.6500.

TABLE 17B
Encoded NOV17 protein sequence.

| | |
|---|---|
| MGQFYRTGAGSGKLQLKVVIYCQLRRDHKVNGEEIIRLIVQKRNVTRSIDRSVGAGQEQV | (SEQ ID NO:44) |
| IMECLKPFYFNYPSLDSEVLDDDRAIDGKDTIILVYKELSRDLASCVPATPAVAERVQGT | |
| VQAMASKGASPKSRQLSQGVKPGSTEGKCGLEPQHRAPLGTLPSGALRRGPPFSRPQNGR | |
| PTDSLHYALGKDTDTQHQPMKAARREAVPCTATGAELPKTMGTQLLHQHDPDVRIGVKED | |
| HFGALRFDCPSRFWTYMRTPALLLCPLTSATLHTGCELPPEEVCGSSSLCLILTVPKCVW | |
| PYQQLQALSLLYFSFGSRRLIALKCNISWSNYIRFHGSASLSPKPSSCIAVMFVLKRLMS | |
| LDYDRKKVSTAAILARIKRTGDGSYKSG | |

A search of sequence databases reveals that the NOV17 amino acid sequence has 25 of 30 amino acid residues (83%) identical to, and 27 of 30 amino acid residues (90%) similar to, the 359 amino acid residue ptnr:SWISSNEW-ACC:O00767 protein from Homo sapiens (Human) (ACYL-COA DESATURASE (EC 1.14.99.5) (STEAROYL-COA DESATURASE) (FATTY ACID DESATURASE) (DELTA(9)-DESATURASE)). Public amino acid databases include the GenBank databases, SwissProt, PDB and PIR.

NOV17 is expressed in at least Adipose, Adrenal Gland/Suprarenal gland, Aorta, Appendix, Artery, Bone, Bone Marrow, Brain, Bronchus, Buccal mucosa, Cartilage, Cerebral Medulla/Cerebral white matter, Cervix, Cochlea, Colon, Cornea, Coronary Artery, Dermis, Epidermis, Foreskin, Frontal Lobe, Gall Bladder, Hair Follicles, Heart, Hippocampus, Hypothalamus, Kidney, Larynx, Left cerebellum, Liver, Lung, Lung Pleura, Lymph node, Lymphoid tissue, Mammary gland/Breast, Myometrium, Ovary, Pancreas, Parathyroid Gland, Parietal Lobe, Parotid Salivary glands, Peripheral Blood, Pharynx, Pituitary Gland, Placenta, Prostate, Respiratory Bronchiole, Retina, Right Cerebellum, Salivary Glands, Skeletal Muscle, Skin, Small Intestine, Spinal Chord, Spleen, Stomach, Substantia Nigra, Synovium/Synovial membrane, Temporal Lobe, Testis, Thalamus, Thymus, Thyroid, Trachea, Umbilical Vein, Urinary Bladder, Uterus, Vein, Vulva. This information was derived by determining the tissue sources of the sequences that were included in the invention including but not limited to SeqCalling sources, Public EST sources, Literature sources, and/or RACE sources.

The disclosed NOV17 polypeptide has homology to the amino acid sequences shown in the BLASTP data listed in Table 17C.

TABLE 17C
BLAST results for NOV17

| Gene Index/ | | Length | Identity | Positives | |
|---|---|---|---|---|---|
| Identifier | Protein/Organism | (aa) | (%) | (%) | Expect |
| gi\|14756057\|ref\|XP_ | hypothetical | 222 | 47/74 | 53/74 | 2e−18 |
| 052325.1\| | protein FLJ14058 | | (63%) | (71%) | |
| (XM_052325) | [Homo sapiens] | | | | |
| gi\|18572532\|ref\|XP_ | KIAA1161 protein | 188 | 59/145 | 72/145 | 7e−17 |
| 088501.1\| | [Homo sapiens] | | (40%) | (48%), | |
| (XM_088501) | | | | | |
| gi\|14722528\|ref\|XP_ | similar to | 129 | 45/94 | 56/94 | 2e−15 |
| 033352.1\| | hypothetical | | (47%) | (58%) | |
| (XM_033352) | protein FLJ14058 | | | | |
| | [Homo sapiens] | | | | |
| gi\|16588389\|gb\|AAL26787.1\| | B lymphocyte | 130 | 48/126 | 56/126 | 3e−10 |
| AF304442_1 | activation- | | (38%) | (44%), | |
| (AF304442) | related protein | | | | |
| | BC-1514 [Homo | | | | |
| | sapiens] | | | | |
| gi\|18562020\|ref\|XP_ | hypothetical | 216 | 36/68 | 39/68 | 2e−8 |
| 087778.1\| | protein XP_087778 | | (52%) | (56%), | |
| (XM_087778) | [Homo sapiens] | | | | |

Fatty acid desaturases (ec 1.14.99.-) are enzymes that catalyze the insertion of a double bond at the delta position of fatty acids. There are two distinct families of fatty acid desaturases which do not seem to be evolutionary related.

Family 1 is composed of:

Stearoyl-coa desaturase (scd) (ec 1.14.99.5). scd is a key regulatory enzyme of unsaturated fatty acid biosynthesis. scd introduces a cis double bond at the delta(9) position of fatty acyl-coa's such as palmitoleoyl- and oleoyl-coa. scd is a membrane-bound enzyme that is thought to function as a part of a multienzyme complex in the endoplasmic reticulum of vertebrates and fungi.

Family 2 is composed of:

Plants stearoyl-acyl-carrier-protein desaturase (ec 1.14.99.6), these enzymes catalyze the introduction of a double bond at the delta(9) position of steraoyl-acp to produce oleoyl-acp. this enzyme is responsible for the conversion of saturated fatty acids to unsaturated fatty acids in the synthesis of vegetable oils.—cyanobacteria desa an enzyme that can introduce a second cis double bond at the delta(12) position of fatty acid bound to membranes glycerolipids. desa is involved in chilling tolerance; the phase transition temperature of lipids of cellular membranes being dependent on the degree of unsaturation of fatty acids of the membrane lipids.

The disclosed NOV17 nucleic acid of the invention encoding an acyl CoA desaturase-like protein includes the nucleic acid whose sequence is provided in Table 17A or a fragment thereof. The invention also includes a mutant or variant nucleic acid any of whose bases may be changed from the corresponding base shown in Table 17A while still encoding a protein that maintains its acyl CoA desaturase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 18 percent of the bases may be so changed.

The disclosed NOV17 protein of the invention includes the acyl CoA desaturase-like protein whose sequence is provided in Table 17B. The invention also includes a mutant or variant protein any of whose residues may be changed from the corresponding residue shown in Table 17B while still encoding a protein that maintains its acyl CoA desaturase-like activities and physiological functions, or a functional fragment thereof. In the mutant or variant protein, up to about 17 percent of the residues may be so changed.

The invention further encompasses antibodies and antibody fragments, such as F_{ab} or (F_{ab})₂, that bind immunospecifically to any of the proteins of the invention.

The above defined information for this invention suggests that this acyl CoA desaturase-like protein (NOV17) may function as a member of a “acyl CoA desaturase family”. Therefore, the NOV17 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV17 nucleic acids and proteins of the invention are useful in potential therapeutic applications implicated in diseases including but not limited to various pathologies and disorders as indicated below. For example, a cDNA encoding the acyl CoA desaturase-like protein (NOV17) may be useful in gene therapy, and the acyl CoA desaturase-like protein (NOV17) may be useful when administered to a subject in need thereof. By way of nonlimiting example, the compositions of the present invention will have efficacy for treatment of patients suffering from Cardiomyopathy, Atherosclerosis, hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Von Hippel-Lindau (VHL) syndrome, Cirrhosis, Transplantation, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, or other pathologies or conditions. The NOV17 nucleic acid encoding the acyl CoA desaturase-like protein of the invention, or fragments thereof, may further be useful in diagnostic applications, wherein the presence or amount of the nucleic acid or the protein are to be assessed.

NOV17 nucleic acids and polypeptides are further useful in the generation of antibodies that bind immuno-specifically to the novel NOV17 substances for use in therapeutic or diagnostic methods. These antibodies may be generated according to methods known in the art, using prediction from hydrophobicity charts, as described in the “Anti-NOVX Antibodies” section below. The disclosed NOV17 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV18

A disclosed NOV18 nucleic acid of 853 nucleotides (also referred to as CG57328-01) encoding a myo-inositol-1 (or 4) monophosphatase-like protein is shown in Table 18A. The start and stop codons are in bold letters.

TABLE 18A
NOV18 nucleotide sequence.

| | |
|---|---|
| ATGGCTGATCCTTGGCAGGAATGCATGGATTATGCAGTAACCCTAGCAAGACAAGCTGGA | (SEQ ID NO:45) |
| GAGGTGGTTCATGATGCTCTTAAAAATGAAGTGAATGTTATCCTGAAAGGTTCTCCAGTT | |
| GATTTGGTAACTGCTACTGACCAAAAAGTTGAAAAAATGCTTATCTCTTCCATAAAGGAA | |
| AAGTATCCATCTCATAGGTATTTTTTTATTGTGAGGAATCTGGCAGCTGGGGAAAAAGGT | |
| GTCTTAACTGACAACCCTACGTGGATCATTGACCCTATTGATGGAACAACTAAGTTTGTC | |
| CATAGATTTCCTTTTGTAGCTGTTTCGATTGGCCTTGTTGTAAATAAGAAGGTAGAATTT | |
| GGAGTTGTGTACAGTTGTGTGGAAGACAAGAGGTACACTGTCAGGAAAGGAAAAGGTGCC | |
| TTTTATAATGGTCAAAAACTACAGGTTTCACAAGAAGGTGATATTACCAAATCACTCTTG | |
| GTGACCGAGCTGGGCTATTGCAGAACATCAGAAATTGTAAGAACTATTCTTTCCAATATG | |
| GAAAAGCTTTCTTGCATTCCTATTCACGGTATCCAGAGTGTTGGAACAGCAGCTACTAAT | |
| ATGTGCATTGCGGCAAGTGGAGGAGCAGAGGCATTTTATGAAATGGGAATTCACTGCTGG | |
| GATATTGCAGTAGCTGCCATTATTGTTACTGAAGCTGGTGGCGTGCTAATGGATGTTACT | |
| GGTGGACCATTCCATTTAATGTCACGGAGAATAATTGCTGCAAATTGTACAGCATTAGCA | |
| GAAAGGATAGCCAAAGAAATTCAGGTAGCACCTTTTCAATGAGATGATGAAGATTAATTA | |
| CAGCAGCCTCATA | |

In a search of public sequence databases, the NOV18 nucleic acid sequence, located on chromsome 8 has 761 of 853 bases (89%) identical to a gb:GENBANK-ID:AF042729|acc:AF042729.2 mRNA from Homo sapiens (Homo sapiens lithium-sensitive myo-inositol monophosphatase A1 (IMPA1) mRNA, complete cds). Public nucleotide databases include all GenBank databases and the GeneSeq patent database.

The disclosed NOV18 polypeptide (SEQ ID NO:46) encoded by SEQ ID NO:45 has 273 amino acid residues and is presented in Table 18B using the one-letter amino acid code. Signal P, Psort and/or Hydropathy results predict that NOV18 has no signal peptide and is likely to be localized in the cytoplasm with a certainty of 0.6500.

TABLE 18B
Encoded NOV18 protein sequence.

| | |
|---|---|
| MADPWQECMDYAVTLARQAGEVVHDALKNEVNVILKGSPVDLVTATDQKVEKMLISSIKE | (SEQ ID NO:46) |
| KYPSHRYFFIVRNLAAGEKGVLTDNPTWIIDPIDGTTKFVHRFPFVAVSIGLVVNKKVEF | |
| GVVYSCVEDKRYTVRKGKGAFYNGQKLQVSQEGDITKSLLVTELGYCRTSEIVRTILSNM | |
| EKLSCIPIHGIQSVGTAATNMCIAASGGAEAFYEMGIHCWDIAVAAIIVTEAGGVLMDVT | |
| GGPFHLMSRRIIAANCTALAERIAKEIQVAPFQ | |

A search of sequence databases reveals that the NOV18 amino acid sequence has 222 of 273 amino acid residues (81%) identical to, and 240 of 273 amino acid residues (87%) similar to, the 277 amino acid residue ptnr:SWISSNEW-ACC:P29218 protein from Homo sapiens (Human) (MYO-INOSITOL-1 (OR 4)-MONOPHOSPHATASE (EC 3.1.3.25) (IMPASE) (IMP) (INOSITOL MONOPHOSPHATASE) (LITHIUM-SENSITIVE MYO-INOSITOL MONOPHOSPHATASE A1)). Public amino acid databases include the GenBank databases, SwissProt, PDB and PIR.

NOV18 is expressed in at least Brain, Lung. This information was derived by determining the tissue sources of the sequences that were included in the invention including but not limited to SeqCalling sources, Public EST sources, Literature sources, and/or RACE sources.

The disclosed NOV18 polypeptide has homology to the amino acid sequences shown in the BLASTP data listed in Table 18C.

TABLE 18C
BLAST results for NOV18

| Gene Index/ | | Length | Identity | Positives | |
|---|---|---|---|---|---|
| Identifier | Protein/Organism | (aa) | (%) | (%) | Expect |
| gi\|18570157\|ref\|XP_ | hypothetical | 512 | | | |
| 095533.1\| | protein XP_095533 | | | | |
| (XM_095533) | [Homo sapiens] | | | | |
| gi\|5031789\|ref\|NP_005527.1\| | inositol (myo)- | 277 | 222/273 | 240/273 | e−124 |
| (NM_005536) | 1 (or 4)- | | (81%) | (87%), | |
| | monophosphatase 1 | | | | |
| | [Homo sapiens] | | | | |
| gi\|3914092\|sp\|P97697\| | Myo-inositol-1 (or | 277 | 222/273 | 240/273 | e−124 |
| MYOP_RAT | 4)- | | (81%) | (87%), | |
| | monophosphatase | | | | |
| | (IMPase) (IMP) | | | | |
| | (Inositol | | | | |
| | monophosphatase) | | | | |
| | (Lithium- | | | | |
| | sensitive myo- | | | | |
| | inositol | | | | |
| | monophosphatase | | | | |
| | A1) | | | | |
| gi\|443382\|pdb\|2HHM\|A | Chain A, Human | 276 | | | |
| | Inositol | | | | |
| | Monophosphatase | | | | |
| | (E.C.3.1.3.25) | | | | |
| | Dimer Complex | | | | |
| | With Gadolinium | | | | |
| | And Sulfate | | | | |
| gi\|127716\|sp\|P20456\| | Myo-inositol-1 (or | 277 | 209/273 | 236/273 | e−119 |
| MYOP_BOVIN | 4)- | | (76%) | (85%), | |
| | monophosphatase | | | | |
| | (IMPase) (IMP) | | | | |
| | (Inositol | | | | |
| | monophosphatase) | | | | |
| | (Lithium- | | | | |
| | sensitive myo- | | | | |
| | inositol | | | | |
| | monophosphatase | | | | |
| | A1) | | | | |

Table 18D lists the domain descriptions from DOMAIN analysis results against NOV18. This indicates that the NOV18 sequence has properties similar to those of other proteins known to contain this domain.

TABLE 18D
Domain Analysis of NOV18

gnl|Pfam|pfam00459, inositol_P, Inositol monophosphatase family
CD-Length = 270 residues, 97.0% aligned
Score = 238 bits (607), Expect = 3e−64

Myo-inositol-1 (or 4)-monophosphatase enzyme catalyzes the reaction:

myo-inositol 1-monophosphate+H(2)O [symbol] myo-inositol+phosphate Acts on both enantiomers of myo-inositol-1-phosphate and myo-inositol 4-phosphate. It does not act on inositol bisphosphates, trisphosphates or tetrakisphosphates. It has been shown that several proteins share two sequence motifs. Two of these proteins are enzymes of the inositol phosphate second messenger signalling pathway:

Vertebrate and plants inositol monophosphatase (EC 3.1.3.25).—Vertebrate inositol polyphosphate 1-phosphatase (EC 3.1.3.57).

Other proteins are:

Bacterial protein cysQ. CysQ could help to control the pool of PAPS (3′-phosphoadenoside 5′-phosphosulfate), or be useful in sulfite synthesis.—Escherichia coli protein suhB. Mutations in suhB results in the enhanced synthesis of heat shock sigma factor (htpR).—Neurospora crassa protein Qa-X. Probably involved in quinate metabolism.—Emericella nidulans protein qutG. Probably involved in quinate metabolism.—Yeast protein HAL2/MET22 involved in salt tolerance as well as methionine biosynthesis.—Yeast hypothetical hypothetical protein YHR046c.—Caenorhabditis elegans hypothetical protein F13G3.5.—A Rhizobium leguminosarum hypothetical protein encoded upstream of the pss gene for exopolysaccharide synthesis.—Methanococcus jannaschii hypothetical protein MJ0109.

It is suggested that these proteins may act by enhancing the synthesis or degradation of phosphorylated messenger molecules. From the X-ray structure of human inositol monophosphatase, it seems that some of the conserved residues are involved in binding a metal ion and/or the phosphate group of the substrate.

The disclosed NOV18 nucleic acid of the invention encoding a myo-inositol-1 (or 4) monophosphatase-like protein includes the nucleic acid whose sequence is provided in Table 18A or a fragment thereof. The invention also includes a mutant or variant nucleic acid any of whose bases may be changed from the corresponding base shown in Table 18A while still encoding a protein that maintains its myo-inositol-1 (or 4) monophosphatase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, LIP to about 11 percent of the bases may be so changed.

The disclosed NOV18 protein of the invention includes the myo-inositol-1 (or 4) monophosphatase-like protein whose sequence is provided in Table 18B. The invention also includes a mutant or variant protein any of whose residues may be changed from the corresponding residue shown in Table 18B while still encoding a protein that maintains its myo-inositol-1 (or 4) monophosphatase-like activities and physiological functions, or a functional fragment thereof. In the mutant or variant protein, up to about 19 percent of the residues may be so changed.

The invention further encompasses antibodies and antibody fragments, such as F_{ab} or (F_{ab})₂, that bind immunospecifically to any of the proteins of the invention.

The above defined information for this invention suggests that this myo-inositol-1 (or 4) monophosphatase-like protein (NOV18) may function as a member of a “myo-inositol-1 (or 4) monophosphatase family”. Therefore, the NOV18 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV18 nucleic acids and proteins of the invention are useful in potential therapeutic applications implicated in diseases including but not limited to various pathologies and disorders as indicated below. For example, a cDNA encoding the myo-inositol-1 (or 4) monophosphatase-like protein (NOV18) may be useful in gene therapy, and the myo-inositol-1 (or 4) monophosphatase-like protein (NOV18) may be useful when administered to a subject in need thereof. By way of nonlimiting example, the compositions of the present invention will have efficacy for treatment of patients suffering from Systemic lupus erythematosus, Autoimmune disease, Asthma, Emphysema, Scleroderma, allergy, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, or other pathologies or conditions. The NOV18 nucleic acid encoding the myo-inositol-1 (or 4) monophosphatase-like protein of the invention, or fragments thereof, may further be useful in diagnostic applications, wherein the presence or amount of the nucleic acid or the protein are to be assessed.

NOV18 nucleic acids and polypeptides are further useful in the generation of antibodies that bind immuno-specifically to the novel NOV18 substances for use in therapeutic or diagnostic methods. These antibodies may be generated according to methods known in the art, using prediction from hydrophobicity charts, as described in the “Anti-NOVX Antibodies” section below. The disclosed NOV18 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV19

A disclosed NOV19 nucleic acid of 2071 nucleotides (also referred to as CG57358-01) encoding a spinster-like protein is shown in Table 19A. The start and stop codons are in bold letters.

TABLE 19A
NOV19 nucleotide sequence.

| | |
|---|---|
| CCCCCCGCCGCCCCGATCCGGGCCGGCATGATGTGCCTGGAATGCGCCTCGGCGGCGGCG | (SEQ ID NO:47) |
| GGCGGCGCGGAGGAGGAGGAGGCGGACGCGGAGCGGCGGCGCCGGCGCCGGGGGGCGCAG | |
| CGAGGGGCTGGCGGTAGCGGTTGCTGCGGGGCGCGGGGCGCGGGCGGCGCTGGAGTCTCG | |
| GCCGCGGGCGATGAGGTGCAGACGCTGTCGGGCAGCGTAAGGCGGGCCCCGACCGGACCC | |
| CCCGGCACCCCCGGCACCCCCGGCTGCGCAGCTACTGCAAAGGGCCCCGGCGCTCAGCAG | |
| CCCAAACCGGCCAGCTTGGGCCGCGGGCGGGGGGCAGCCGCCGCCATCCTCAGCTTGGGC | |
| AACGTGCTCAACTACCTGGACAGGTACACCGTGGCAGGCGTCCTTCTGGACATCCAGCAG | |
| CACTTTGGGGTCAAGGACCGAGGCGCCGGCCTGCTGCAGTCAGTGTTCATCTGTAGCTTC | |
| ATGGTGGCTGCCCCCATCTTCGGCTACCTGGGCGACCGCTTCAACAGGAAGGTGATTCTC | |
| AGCTGCGGCATTTTCTTCTGGTCGGCCGTCACCTTCTCCAGCTCCTTCATTCCCCAGCAG | |
| TACTTCTGGCTGCTGGTCCTGTCCCGGGGGCTGGTGGGCATCGGGGAGGCCAGCTACTCC | |
| ACCATCGCCCCCACTATCATTGGCGACCTCTTCACCAAGAACACGCGTACGCTCATGCTG | |
| TCCGTCTTCTACTTCGCCATCCCACTGGGCAGTGGCCTGGGCTACATTACTGGCTCCAGC | |
| GTGAAGCAGGCAGCCGGAGACTGGCACTGGGCATTGCGGGTGTCCCCTGTCCTGGGCATG | |
| ATCACAGGAACACTCATCCTCATTCTGGTCCCAGCCACTAAAAGGGGTCATGCCGACCAG | |
| CTCGGGGACCAGCTCAAGGCCCGGACCTCATGGCTCCGAGATATGAAGGCCCTGATTCGA | |
| AACCGCAGCTACGTCTTCTCCTCCCTGGCCACGTCGGCTGTCTCCTTCGCCACGGGGGCC | |
| CTGGGCATGTGGATCCCGCTCTACCTGCACCGCGCCCAAGTTGTGCAGAAGACAGCAGAG | |
| ACGTGCAACAGCCCGCCCTGTGGGGCCAAGGACAGCCTCATCTTTGGGGCCATCACCTGC | |
| TTTACGGGATTTCTGGGCGTGGTCACGGGGGCAGGAGCCACGCGCTGGTGCCGCCTGAAG | |
| ACCCAGCGGGCCGACCCACTGGTGTGTGCCGTGGGCATGCTGGGCTCTGCCATCTTCATC | |
| TGCCTGATCTTCGTGGCTGCCAAGAGCAGCATCGTAGGAGCCTATATCTGTATCTTCGTC | |
| GGGGAGACGCTGCTGTTTTCTAACTGGGCCATCACTGCAGACATCCTCATGTACGTGGTC | |
| ATCCCCACGCGGCGCGCCACTGCCGTGGCCTTGCAGAGCTTCACCTCCCACCTGCTGGGG | |
| GACGCCGGGAGCCCCTACCTCATTGGCTTTATCTCAGACCTGATCCGCCAGAGCACTAAG | |
| GACTCCCCGCTCTGGGAGTTCCTGAGCCTGGGCTACGCGCTCATGCTCTGCCCTTTCGTC | |
| GTGGTCCTGGGCGGCATGTTCTTCCTCGCCACTGCGCTCTTCTTCGTCAGCGACCGCGCC | |
| AGGGCTGAGCAGCACCTGGGGGAGAGACGGGCGGGGGTCAGGGTGGTGCATCAGCGGGGG | |
| CCGGGCCCGGGCACTGCTCTGGCACATCGTGTCGTGGGGGCCAGCTGACCGGAGGTGCTG | |
| GGCAGGGACCTCGTCAGCCCCAGGGGGAGATGGGAGAGCCCAGGGGTGGGGAGAGGAGAG | |
| AGAGAGGAGTAAAGAGGAAAGGAGAAAGAAGTCAGAAAGTAAGAGGAAGGGGAGGGGCCC | |
| CAGCTTTGAAAACCACTAAGTCCAGAGACAAACCCAAGTCTGGATCCACCAGACACCCCC | |
| GTGGCCTCCCACAGCTCCAGGCTGACCCTGGCACTGGGCCTCAGGGCTGGACCCCAGCAA | |
| CCAGTGGGTGCACTGAGTGCATGGGAGGTCTGTACCCTCCCCGCCCCACCCCAGGGCAGG | |
| GCTCACGGTGGCTATCACGGTCCCTGCTTCC | |

In a search of public sequence databases, the NOV19 nucleic acid sequence, located on chromsome 17 has 290 of 431 bases (67%) identical to a gb:GENBANK-ID:E12646|acc:E12646.1 mRNA from Homo sapiens (cDNA encoding cell growth inhibiting factor). Public nucleotide databases include all GenBank databases and the GeneSeq patent database.

The disclosed NOV19 polypeptide (SEQ ID NO:48) encoded by SEQ ID NO:47 has 566 amino acid residues and is presented in Table 19B using the one-letter amino acid code. Signal P, Psort and/or Hydropathy results predict that NOV19 has no signal peptide and is likely to be localized at the plasma membrane with a certainty of 0.6000.

TABLE 19B
Encoded NOV19 protein sequence.

| | |
|---|---|
| MMCLECASAAAGGAEEEEADAERRRRRRGAQRGAGGSGCCGARGAGGAGVSAAGDEVQTL | (SEQ ID NO:48) |
| SGSVRRAPTGPPGTPGTPGCAATAKGPGAQQPKPASLGRGRGAAAAILSLGNVLNYLDRY | |
| TVAGVLLDIQQHFGVKDRGAGLLQSVFICSFMVAAPIFGYLGDRFNRKVILSCGIFFWSA | |
| VTFSSSFIPQQYFWLLVLSRGLVGIGEASYSTIAPTIIGDLFTKNTRTLMLSVFYFAIPL | |
| GSGLGYITGSSVKQAAGDWHWALRVSPVLGMITGTLILILVPATKRGHADQLGDQLKART | |
| SWLRDMKALIRNRSYVFSSLATSAVSFATGALGMWIPLYLHRAQVVQKTAETCNSPPCGA | |
| KDSLIFGAITCFTGFLGVVTGAGATRWCRLKTQRADPLVCAVGMLGSAIFICLIFVAAKS | |
| SIVGAYICIFVGETLLFSNWAITADILMYVVIPTRRATAVALQSFTSHLLGDAGSPYLIG | |
| FISDLIRQSTKDSPLWEFLSLGYALMLCPFVVVLGGMFFLATALFFVSDRARAEQHLGER | |
| RAGVRVVHQRGPGPGTALAHRVVGAS | |

A search of sequence databases reveals that the NOV19 amino acid sequence has 268 of 495 amino acid residues (54%) identical to, and 330 of 495 amino acid residues (66%) similar to, the 528 amino acid residue ptnr:TREMBLNEW-ACC:AAG43830 protein from Homo sapiens (Human) (SPINSTER-LIKE PROTEIN). Public amino acid databases include the GenBank databases, SwissProt, PDB and PIR.

NOV is expressed in at least brain and heart. This information was derived by determining the tissue sources of the sequences that were included in the invention including but not limited to SeqCalling sources, Public EST sources, Literature sources, and/or RACE sources.

The disclosed NOV19 polypeptide has homology to the amino acid sequences shown in the BLASTP data listed in Table 19C.

TABLE 19C
BLAST results for NOV19

| Gene Index/ | | Length | Identity | Positives | |
|---|---|---|---|---|---|
| Identifier | Protein/Organism | (aa) | (%) | (%) | Expect |
| gi\|15079262\|gb\|AAH11467.1\| | protein for | 590 | 514/539 | 523/539 | 0.0 |
| AAH11467 | IMAGE: 3154539) | | (95%) | (96%) | |
| (BC011467) | [Mus musculus] | | | | |
| gi\|18448989\|gb\|AAL69987.1\| | not really | 506 | 248/431 | 312/431 | e−133 |
| AF465772_1 | started [Danio | | (57%) | (71%), | |
| (AF465772) | rerio] | | | | |
| gi\|13544043\|gb\|AAH06156.1\| | (protein for | 524 | 266/478 | 327/478 | e−129 |
| AAH06156 | IMAGE: 3627317) | | (55%) | (67%), | |
| (BC006156) | [Homo sapiens] | | | | |
| gi\|14042968\|ref\|NP_ | spinster-like | 528 | 268/495 | 330/495 | e−129 |
| 114427.1\| | protein [Homo | | (54%) | (66%), | |
| (NM_032038) | sapiens] | | | | |
| gi\|12963795\|ref\|NP_ | spinster-like | 528 | 266/510 | 330/510 | e−126 |
| 076201.1\| | protein [Mus | | (52%) | (64%), | |
| (NM_023712) | musculus] | | | | |

Table 19D-E lists the domain descriptions from DOMAIN analysis results against NOV19. This indicates that the NOV sequence has properties similar to those of other proteins known to contain this domain.

TABLE 19D
Domain Analysis of NOV19

gnl|Pfam|pfam00083, sugar_tr, Sugar (and other) transporter.
CD-Length = 447 residues, 28.2% aligned
Score = 55.1 bits (131), Expect = 1e−08

TABLE 19E
Domain Analysis of NOV19

gnl|Pfam|pfam03137, OATP_C, Organic Anion Transporter Polypeptide
(OATP) family, C-terminus. This family consists of several eukaryotic
Organic-Anion-Transporting Polypeptides (OATPs). Several have been
identified mostly in human and rat. Different OATPs vary in tissue
distribution and substrate specificity. Since the numbering of
different OATPs in particular species was based originally on the
order of discovery, similarly numbered OATPs in humans and rats did
not necessarily correspond in function, tissue distribution and
substrate specificity (in spite of the name, some OATPs also transport
organic cations and neutral molecules). Thus, Tamai et al. initiated
the current scheme of using digits for rat OATPs and letters for human
ones. Prostaglandin transporter (PGT) proteins (e.g. Pfam: Q92959) are
also considered to be OATP family members. In addition, the
methotrexate transporter OATK (Pfam: P70502) is closely related to
OATPs. This family aligns residues towards the C-terminus. The family
OATP_N aligns residues from similar proteins towards the N-terminus.
This family also includes several predicted proteins from
Caenorhabditis elegans and Drosophila melanogaster. This similarity
was not previously noted. Note: Members of this family are described
(in the Swiss-Prot database) as belonging to the SLC21 family of
transporters.
CD-Length = 375 residues, 18.9% aligned
Score = 37.7 bits (86), Expect = 0.002

NOV19 is a homolog of the spinster-like proteins in human and mouse. Spinster is a novel membrane protein in Drosophila, mutants of which exhibit accumulation of ceroid lipofuscin and neural degeneration (See Nakano et al., Genbank entry for AAG43830.1). Accumulation of ceroid lipofuscin occurs in several hereditary disorders that are probably related to lysosomal storage defects. The pigment makes fibroblasts in vitro more susceptible to oxidative stress, leading to apoptosis (See Terman et al., Exp Gerontol 1999 September;34(6):755-70). It Is also a component of atherogenic lesions in arteries (See Hoffe and Hoppe, Curr Opin Lipidol 1995 October;6(5):317-25). In neurons, accumulation of the pigment leads to neurodegeneration, even leading to death in some cases (See Dyken and Wisniewski, Am J Med Genet Jun. 5, 1995;57(2):150-4). This is seen both in inherited forms of human ceroid lipofuscinoses (See Jagadha et al., Acta Neuropathol (Berl) 1988;75(3):233-40) and in a cathepsin-D-deficient mouse model (See Koike et al., J Neurosci Sep. 15, 2000;20(18):6898-906). In at least one case, neuronal deposition of ceroid lipofuscin was also correlated with accumulation in the myocardium (See Jay and Haslam, J Inherit Metab Dis 1995;18(3):359-60).

The disclosed NOV19 nucleic acid of the invention encoding a spinster-like protein includes the nucleic acid whose sequence is provided in Table 19A or a fragment thereof. The invention also includes a mutant or variant nucleic acid any of whose bases may be changed from the corresponding base shown in Table 19A while still encoding a protein that maintains its spinster-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 33 percent of the bases may be so changed.

The disclosed NOV19 protein of the invention includes the spinster-like protein whose sequence is provided in Table 19B. The invention also includes a mutant or variant protein any of whose residues may be changed from the corresponding residue shown in Table 19B while still encoding a protein that maintains its spinster-like activities and physiological functions, or a functional fragment thereof. In the mutant or variant protein, up to about 46 percent of the residues may be so changed.

The invention further encompasses antibodies and antibody fragments, such as F_{ab} or (F_{ab})₂ that bind immunospecifically to any of the proteins of the invention.

The above defined information for this invention suggests that this spinster-like protein (NOV19) may function as a member of a “spinster family”. Therefore, the NOV19 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV19 nucleic acids and proteins of the invention are useful in potential therapeutic applications implicated in diseases including but not limited to various pathologies and disorders as indicated below. For example, a cDNA encoding the spinster-like protein (NOV19) may be useful in gene therapy, and the spinster-like protein (NOV19) may be useful when administered to a subject in need thereof. By way of nonlimiting example, the compositions of the present invention will have efficacy for treatment of patients suffering from cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, Von Hippel-Lindau (VHL) syndrome, Alzheimer's disease, stroke, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cancer, tissue degeneration, bacterial/viral/parasitic infection, or other pathologies or conditions. The NOV19 nucleic acid encoding the spinster-like protein of the invention, or fragments thereof, may further be useful in diagnostic applications, wherein the presence or amount of the nucleic acid or the protein are to be assessed.

NOV19 nucleic acids and polypeptides are further useful in the generation of antibodies that bind immuno-specifically to the novel NOV19 substances for use in therapeutic or diagnostic methods. These antibodies may be generated according to methods known in the art, using prediction from hydrophobicity charts, as described in the “Anti-NOVX Antibodies” section below. The disclosed NOV19 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV20

A disclosed NOV20 nucleic acid of 752 nucleotides (also referred to as CG57695-01) encoding a casein-like protein is shown in Table 20A. The start and stop codons are in bold letters.

TABLE 20A
NOV20 nucleotide sequence.

| | |
|---|---|
| CAAACAACCATGAAGTTCTTCATCTTTACCTGCCTTTTGGCTGTTGCTCTGGCACATCAT | (SEQ ID NO:49) |
| GAACTTAAACACGTTTACAAGAAAAAAACAAACAACAACGTGAGTGACAAATACAGAAAT | |
| GTGAAAAACCAGATTTCTTCTCCTCAGGAGGACAAAGTTAGAGGTAATTTTCATTCAAAT | |
| AAAATAAAATTAATACCACTTAGTAGTGTTTTATTTTTGTATATTTGTATACAAATTAAT | |
| TTTTTTTCTTACCAGGAAGTTAAGCACACTGTGGATCAAAAGCACTACGTAAAACAACTG | |
| AACAAAATCAACCCATTTTATCAGAAGTGGAACTTCCTCCCATTTCTTCAGGTTCCTTAT | |
| CAACATCACATTTTTATAAACCCAGGAGATCAGCATAAGACAAGTGTCTACCCCTTTGTT | |
| CCCACTAAATATATACAGTGGCCAGGCTCAGTGGCTCAGGCCTTCTTGTTTTATTCCTTT | |
| AAGGAAACACCAAAAAAGACTGTTGATATGGTAAAGTATTGTTTCTATCAGAAAACTGAG | |
| CTGACTGAAGAAGAAAAGAATGACCAAAAACATCTGAACAAAATCAACCAGTATTATCAG | |
| TTCACCTTGCCCCAATATGTAAAAGCTGTTTATCAATATCACAAAATTATGAAACCATGG | |
| AAAAACATGAAGACAAATGCTTACCAAGTTATCCCCACTCTGGTGAGTGCTCTCTTTTTA | |
| TTTGCAACTTAAAAATAGTTATCTGTTGTGCT | |

In a search of public sequence databases, the NOV20 nucleic acid sequence, located on chromsome 4 has 291 of 445 bases (65%) identical to a gb:GENBANK-ID:CHI249995|acc:AJ249995.1 mRNA from Capra hircus (Capra hircus mRNA for alpha s2-casein (cis1s2 gene)). Public nucleotide databases include all GenBank databases and the GeneSeq patent database.

The disclosed NOV20 polypeptide (SEQ ID NO:50) encoded by SEQ ID NO:49 has 240 amino acid residues and is presented in Table 20B using the one-letter amino acid code. Signal P, Psort and/or Hydropathy results predict that NOV20 has a signal peptide and is likely to be localized extracellularly with a certainty of 0.5140.

TABLE 20B
Encoded NOV20 protein sequence.

| | |
|---|---|
| MKFFIFTCLLAVALAHHELKHVYKKKTNNNVSDKYRNVKNQISSPQEDKVRGNFHSNKIK | (SEQ ID NO:50) |
| LIPLSSVLFLYICIQINFFSYQEVKHTVDQKHYVKQLNKINPFYQKWNFLPFLQVPYQHQ | |
| IFINPGDQHKTSVYPFVPTKYIQWPGSVAQAFLFYSFKETPKKTVDMVKYCFYQKTELTE | |
| EEKNDQKHLNKINQYYQFTLPQYVKAVYQYHKIMKPWKNMKTNAYQVIPTLVSALFLFAT | |

A search of sequence databases reveals that the NOV20 amino acid sequence has 112 of 232 amino acid residues (48%) identical to, and 142 of 232 amino acid residues (61%) similar to, the 235 amino acid residue ptnr:pir-id:A48383 protein from pig (alpha s2-casein). Public amino acid databases include the GenBank databases, SwissProt, PDB and PIR.

NOV20 is expressed in at least lung, testis, and b-cell. This information was derived by determining the tissue sources of the sequences that were included in the invention including but not limited to SeqCalling sources, Public EST sources, Literature sources, and/or RACE sources.

The disclosed NOV20 polypeptide has homology to the amino acid sequences shown in the BLASTP data listed in Table 20C.

TABLE 20C
BLAST results for NOV20

| Gene Index/ | | Length | Identity | Positives | |
|---|---|---|---|---|---|
| Identifier | Protein/Organism | (aa) | (%) | (%) | Expect |
| gi\|477186\|pir\|\|A48383 | alpha s2-casein - pig | 235 | 111/234 | 141/234 | 2e−37 |
| | | | (47%) | (59%) | |
| gi\|729044\|sp\|P39036\| | Alpha-S2 casein | 235 | 109/234 | 138/234 | 2e−35 |
| CAS2_PIG | precursor | | (46%) | (58%) | |
| gi\|115658\|sp\|P04654\| | Alpha-S2 casein | 223 | 100/227 | 128/227 | 3e−28 |
| CAS2_SHEEP | precursor | | (44%) | (56%) | |
| gi\|416751\|sp\|P33049\| | Alpha-S2 casein | 223 | 96/227 | 125/227 | 4e−28 |
| CAS2_CAPHI | precursor (Alpha- | | (42%) | (54%) | |
| | S2-CN) | | | | |
| gi\|423305\|pir\|\|S33881 | alphas2-casein B - goat | 223 | 96/227 | 125/227 | 4e−28 |
| | | | (42%) | (54%), | |

Table 20D lists the domain descriptions from DOMAIN analysis results against NOV20. This indicates that the NOV20 sequence has properties similar to those of other proteins known to contain this domain.

TABLE 20D
Domain Analysis of NOV20

gnl|Pfam|pfam00363, caseins, Casein.
CD-Length = 84 residues, 71.4% aligned
Score = 44.3 bits (103), Expect = 8e−06

NOV20 has homology to pig alpha S casein. Caseins are the major protein constituent of milk. Caseins can be classified into two families; the first consists of the kappa-caseins, and the second groups the alpha-s1, alpha-s2, and beta-caseins. The alpha/beta caseins are a rapidly diverging family of proteins. However two regions are conserved: a cluster of phosphorylated serine residues and the signal sequence. Alpha-s2 casein is known as epsilon-casein in mouse, gamma-casein in rat and cascin-A in guinea pig. Alpha-s1 cascin is known as alpha-casein in rat and rabbit and as casein-B in guinea-pig.

Milk casein can be separated by urea starch electrophoresis into 3 regions, alpha, beta (115460), and kappa (601695) casein. Alpha and beta variants are present in the human population. Voglino and Ponzone (See Voglino, G. F.; Ponzone, A.: Nature N.B. 238: 149, 1972) postulated 2 biallelic systems. In Italy the frequency of the 2 alpha alleles was 0.908 and 0.092; 2 beta alleles had a frequency of 0.678 and 0.322.

Fujiwara et al. (See Flujiwara, Y.et al., Hum. Genet. 99: 368-373, 1997) found that the human alpha-S1, beta-, and kappa-casein genes are closely linked and arranged in that order. By fluorescence in situ hybridization, they demonstrated that the casein gene family is localized to 4q21.1. Rijnkels et al. (See Rijnkels, M., et al., Mammalian Genome 8: 285-286, 1997) concluded that the human ‘locus’ comprises at least 4 casein genes: 3 genes encoding calcium-sensitive, casein-like genes, and 1 kappa-casein gene, in the order alpha-s1—beta—alpha-s2—kappa. The approximate size of the human casein gene locus is 350 kb. Chen et al. (See Chen, C. -S. et al., Cytogenet. Cell Genet. 69: 260-265, 1995.) suggested that the casein cluster is located within 700 kb of the albumin (103600) gene cluster, which is located on 4q13.

The disclosed NOV20 nucleic acid of the invention encoding a cascin-like protein includes the nucleic acid whose sequence is provided in Table 20A or a fragment thereof. The invention also includes a mutant or variant nucleic acid any of whose bases may be changed from the corresponding base shown in Table 20A while still encoding a protein that maintains its casein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 35 percent of the bases may be so changed.

The disclosed NOV20 protein of the invention includes the casein-like protein whose sequence is provided in Table 20B. The invention also includes a mutant or variant protein any of whose residues may be changed from the corresponding residue shown in Table 20B while still encoding a protein that maintains its casein-like activities and physiological functions, or a functional fragment thereof. In the mutant or variant protein, up to about 52 percent of the residues may be so changed.

The invention further encompasses antibodies and antibody fragments, such as F_{ab} or (F_{ab})₂, that bind immunospecifically to any of the proteins of the invention.

The above defined information for this invention suggests that this casein-like protein (NOV20) may function as a member of a “casein family”. Therefore, the NOV20 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV20 nucleic acids and proteins of the invention are useful in potential therapeutic applications implicated in diseases including but not limited to various pathologies and disorders as indicated below. For example, a cDNA encoding the cascin-like protein (NOV20) may be useful in gene therapy, and the casein-like protein (NOV20) may be useful when administered to a subject in need thereof. By way of nonlimiting example, the compositions of the present invention will have efficacy for treatment of patients suffering from fertility, hypogonadism, systemic lupus erythematosus, autoimmune disease, asthma, emphysema, scleroderma, allergy, ARDS, hemophilia, hypercoagulation, idiopathic thrombocytopenic purpura, autoimmune disease, allergies, immunodeficiencies, transplantation, graft versus host disease (GVHD), lymphaedema, or other pathologies or conditions. The NOV20 nucleic acid encoding the casein-like protein of the invention, or fragments thereof, may further be useful in diagnostic applications, wherein the presence or amount of the nucleic acid or the protein are to be assessed.

NOV20 nucleic acids and polypeptides are further useful in the generation of antibodies that bind immuno-specifically to the novel NOV20 substances for use in therapeutic or diagnostic methods. These antibodies may be generated according to methods known in the art, using prediction from hydrophobicity charts, as described in the “Anti-NOVX Antibodies” section below. The disclosed NOV20 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV21

A disclosed NOV21 nucleic acid of 1704 nucleotides (also referred to as CG57654-01) encoding a gamma-aminobutyric acid receptor-like protein is shown in Table 21A. The start and stop codons are in bold letters.

TABLE 21A
NOV21 nucleotide sequence.

| | |
|---|---|
| CCTGACGCTTTGATGGTATCTGCAAGCGTTTTTGCTGATCTTATCTCTGCCCCCTGAATA | (SEQ ID NO:51) |
| TTAATTCCCTAATCTGGTAGCAATCCATCTCCCCAGTGAAGGACCTACTAGAGGCAGGTG | |
| GGGGGAGCCACCATCAGATCATCAAGCATAAGAATAATACAAAGGGGAGGGATTCTTCTG | |
| CAACCAAGAGGCAAGAGGCGAGAGAAGGAAAAAAAAAAAAAAAGCGATGAGTTCACCAAA | |
| TATATCGAGCACAGGAAGCTCAGTCTACTCGACTCCTGTATTTTCACAGAAAATGACGGT | |
| GTGGATTCTCCTCCTGCTGTCGCTCTACCCTGGCTTCACTAGCCAGAAATCTGATGATGA | |
| CTATGAAGATTATGCTTCTAACAAAACATGGGTCTTGACTCCAAAAGTTCCTGAGGGTGA | |
| TGTCACTGTCATCTTAAACAACCTGCTGGAAGGATATGACAATAAACTTCGGCCTGATAT | |
| AGGAGTGAAGCCAACGTTAATTCACACAGACATGTATGTGAATAGCATTGGTCCAGTGAA | |
| CGCTATCAATATGGAATACACTATTGATATATTTTTTGCGCAAATGTGGTATGACAGACG | |
| TTTGAAATTTAACAGCACCATTAAAGTCCTCCGATTGAACAGCAACATGGTGGGGAAAAT | |
| CTGGATTCCAGACACTTTCTTCAGAAATTCCAAAAAAGCTGATGCACACTGGATCACCAC | |
| CCCCAACAGGATGCTGAGAATTTGGAATGATGGTCGAGTGCTCTACTCCCTAAGGTTGAC | |
| AATTGATGCTGAGTGCCAATTACAATTGCACAATTTTCCAATGGATGAACACTCCTGCCC | |
| CTTCGAGTTCTCCAGTTATGGCTATCCACGTGAAGAAATTGTTTATCAATGGAAGCGAAG | |
| TTCTGTTGAAGTGGGCGACACAAGATCCTGGAGGCTTTATCAATTCTCATTTGTTGGTCT | |
| AAGAAATACCACCGAAGTAGTGAAGACAACTTCCGGAGATTATGTGGTCATGTCTGTCTA | |
| CTTTGATCTGAGCAGAAGAATGGGATACTTTACCATCCAGACCTATATCCCCTGCACACT | |
| CATTGTCGTCCTATCCTGGGTGTCTTTCTGGATCAATAAGGATGCTGTTCCAGCCAGAAC | |
| ATCTTTAGGTATCACCACTGTCCTGACAATGACCACCCTCAGCACCATTGCCCGGAAATC | |
| GCTCCCCAAGGTCTCCTATGTCACAGCGATGGATCTCTTTGTATCTGTTTGTTTCATCTT | |
| TGTCTTCTCTGCTCTGGTGGAGTATGGCACCTTGCATTATTTTGTCAGCAACCGGAAACC | |
| AAGCAAGGACAAAGATAAAAAGAAGAAAAACCCTCTTCTTCGGATGTTTTCCTTCAAGGC | |
| CCCTACCATTGATATCCGCCCAAGATCAGCAACCATTCAAATGAATAATGCTACACACCT | |
| TCAAGAGAGAGATGAAGAGTACGGCTATGAGTGTCTGGACGGCAAGGACTGTGCCAGTTT | |
| TTTCTGCTGTTTTGAAGATTGTCGAACAGGAGCTTGGAGACATGGGAGGATACATATCCG | |
| CATTGCCAAAATGGACTCCTATGCTCGCATCTTCTTCCCCACTGCCTTCTGCCTGTTTAA | |
| TCTGGTCTATTGGGTCTCCTACCTCTACCTGTGAGGAGGTATGGGTTTTACTGATATGGT | |
| TCTTATTCACTGAGTCTCATGGAG | |

In a search of public sequence databases, the NOV21 nucleic acid sequence, located on chromsome 5 has 1379 of 1398 bases (98%) identical to a gb:GENBANK-ID:HSGABAAS|acc:X15376.1 mRNA from Homo sapiens (Human mRNA for GABA-A receptor, gamma 2 subunit). Public nucleotide databases include all GenBank databases and the GeneSeq patent database.

The disclosed NOV21 polypeptide (SEQ ID NO:52) encoded by SEQ ID NO:51 has 475 amino acid residues and is presented in Table 21B using the one-letter amino acid code. Signal P, Psort and/or Hydropathy results predict that NOV21 has a signal peptide and is likely to be localized at the plasma membrane with a certainty of 0.6000. The most likely cleavage site for a NOV21 peptide is between amino acids 39 and 40.

TABLE 21B
Encoded NOV21 protein sequence.

| | |
|---|---|
| MSSPNIWSTGSSVYSTPVFSQKMTVWILLLLSLYPGFTSQKSDDDYEDYASNKTWVLTPK | (SEQ ID NO:52) |
| VPEGDVTVILNNLLEGYDNKLRPDIGVKPTLTHTDMYVNSIGPVNAINMEYTIDIFFAQM | |
| WYDRRLKFNSTIKVLRLNSNMVGKIWIPDTFFRNSKKADAHWITTPNRMLRIWNDGRVLY | |
| SLRLTIDAECQLQLHNFPMDEHSCPLEFSSYGYPREEIVYQWKRSSVEVGDTRSWRLYQF | |
| SFVGLRNTTEVVKTTSGDYVVMSVYFDLSRRMGYFTIQTYIPCTLIVVLSWVSFWINKDA | |
| VPARTSLGITTVLTMTTLSTIARKSLPKVSYVTAMDLFVSVCFIFVFSALVEYGTLHYFV | |
| SNRKPSKDKDKKKKNPLLRMFSFKAPTIDIRPRSATIQMNNATHLQERDEEYGYECLDGK | |
| DCASFFCCFEDCRTGAWRHGRIHIRIAKMDSYARIFFPTAFCLFNLVYWVSYLYL | |

A search of sequence databases reveals that the NOV21 amino acid sequence has 467 of 475 amino acid residues (98%) identical to, and 467 of 475 amino acid residues (98%) similar to, the 467 amino acid residue ptnr:SWISSNEW-ACC:P18507 protein from Homo sapiens (Human) (GAMMA-AMINOBUTYRIC-ACID RECEPTOR GAMMA-2 SUBUNIT PRECURSOR (GABA(A) RECEPTOR)). Public amino acid databases include the GenBank databases, SwissProt, PDB and PIR.

NOV21 is expressed in at least Adrenal Gland/Suprarenal gland, Brain, Hippocampus, Pituitary Gland, and Right Cerebellum. Expression information was derived from the tissue sources of the sequences that were included in the derivation of the sequence of CG57654-01. The sequence is predicted to be expressed in the following tissues because of the expression pattern of (GENBANK-ID: gb:GENBANK-ID:HSGABAAS|acc:X15376.1) a closely related Human mRNA for GABA-A receptor, gamma 2 subunit homolog in species Homo sapiens: fetal brain. This information was derived by determining the tissue sources of the sequences that were included in the invention including but not limited to SeqCalling sources, Public EST sources, Literature sources, and/or RACE sources.

The disclosed NOV21 polypeptide has homology to the amino acid sequences shown in the BLASTP data listed in Table 21C.

TABLE 21C
BLAST results for NOV21

| Gene Index/ | | Length | Identity | Positives | |
|---|---|---|---|---|---|
| Identifier | Protein/Organism | (aa) | (%) | (%) | Expect |
| gi\|4557611\|ref\|NP_000807.1\| | gamma- | 467 | 467/475 | 467/475 | 0.0 |
| (NM_000816) | aminobutyric acid | | (98%) | (98%) | |
| | A receptor, gamma | | | | |
| | 2 precursor [Homo | | | | |
| | sapiens] | | | | |
| gi\|5738138\|gb\|AAD50273.1\| | gamma- | 467 | 465/475 | 466/475 | 0.0 |
| (AF165124) | aminobutyric acid | | (97%) | (97%) | |
| | A receptor gamma | | | | |
| | 2 [Homo sapiens] | | | | |
| gi\|120784\|sp\|P22300\| | GAMMA- | 475 | 467/475 | 470/475 | 0.0 |
| GAC2_BOVIN | AMINOBUTYRIC-ACID | | (98%) | (98%) | |
| | RECEPTOR GAMMA-2 | | | | |
| | SUBUNIT PRECURSOR | | | | |
| | (GABA(A) | | | | |
| | RECEPTOR) | | | | |
| gi\|108682\|pir\|\|B39272 | gamma- | 475 | 467/475 | 470/475 | 0.0 |
| | aminobutyric acid | | (98%) | (98%) | |
| | receptor A gamma- | | | | |
| | 2L chain - bovine | | | | |
| gi\|6679915\|ref\|NP_032099.1\| | gamma- | 474 | 469/475 | 471/475 | 0.0 |
| (NM_008073) | aminobutyric acid | | (98%) | (98%) | |
| | (GABA-A) | | | | |
| | receptor, subunit | | | | |
| | gamma 2 [Mus | | | | |
| | musculus] | | | | |

Table 21D lists the domain descriptions from DOMAIN analysis results against NOV21. This indicates that the NOV21 sequence has properties similar to those of other proteins known to contain this domain.

TABLE 21D
Domain Analysis of NOV21

gnl|Pfam|pfam02931, Neur_chan_LBD, Neurotransmitter-gated
ion-channel ligand binding domain. This family is the extracellular
ligand binding domain of these ion channels. This domain forms a
pentameric arrangement in the known structure.
CD-Length = 200 residues, 91.5% aligned
Score = 170 bits (431), Expect = 1e−43

Neurotransmission effected by GABA (gamma-aminobutyric acid) is predominantly mediated by a gated chloride channel intrinsic to the GABA receptor. This heterooligomeric receptor exists in most inhibitory synapses in the vertebrate central nervous system (CNS) and can be regulated by clinically important compounds such as benzodiazepines and barbiturates. The primary structures of GABAA receptor alpha- and beta-subunits have been deduced from cloned complementary DNAs. Co-expression of these subunits in heterologous systems generates receptors which display much of the pharmacology of their neural counterparts, including potentiation by barbiturates. Conspicuously, however, they lack binding sites for, and consistent electrophysiological responses to, benzodiazepinies. (See Pritchett et al. Nature 1989;338:582-5) reported the isolation of a cloned cDNA encoding a new GABAA receptor subunit, termed gamma 2, which shares approximately 40% sequence identity with alpha- and beta-subunits and whose messenger RNA is prominently localized in neuronal subpopulations throughout the CNS. Importantly, coexpression of the gamma 2 subunit with alpha 1 and beta 1 subunits produces GABAA receptors displaying high-affinity binding for central benzodiazepine receptor ligands.

The disclosed NOV21 nucleic acid of the invention encoding a gamma-aminobutyric acid receptor-like protein includes the nucleic acid whose sequence is provided in Table 21A or a fragment thereof. The invention also includes a mutant or variant nucleic acid any of whose bases may be changed from the corresponding base shown in Table 21A while still encoding a protein that maintains its gamma-aminobutyric acid receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2 percent of the bases may be so changed.

The disclosed NOV21 protein of the invention includes the gamma-aminobutyric acid receptor-like protein whose sequence is provided in Table 21B. The invention also includes a mutant or variant protein any of whose residues may be changed from the corresponding residue shown in Table 21B while still encoding a protein that maintains its gamma-aminobutyric acid receptor-like activities and physiological functions, or a functional fragment thereof. In the mutant or variant protein, up to about 2 percent of the residues may be so changed.

The invention further encompasses antibodies and antibody fragments, such as F_{ab} or (F_{ab})₂, that bind immunospecifically to any of the proteins of the invention.

The above defined information for this invention suggests that this gamma-aminobutyric acid receptor-like protein (NOV21) may function as a member of a “gamma-aminobutyric acid receptor family”. Therefore, the NOV21 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV21 nucleic acids and proteins of the invention are useful in potential therapeutic applications implicated in diseases including but not limited to various pathologies and disorders as indicated below. For example, a cDNA encoding the gamma-aminobutyric acid receptor-like protein (NOV21) may be useful in gene therapy, and the gamma-aminobutyric acid receptor-like protein (NOV21) may be useful when administered to a subject in need thereof. By way of nonlimiting example, the compositions of the present invention will have efficacy for treatment of patients suffering from adrenoleukodystrophy, congenital adrenal hyperplasia, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, endocrine dysfunctions, diabetes, obesity, growth and reproductive disorders, or other pathologies or conditions. The NOV21 nucleic acid encoding the gamma-aminobutyric acid receptor-like protein of the invention, or fragments thereof, may further be useful in diagnostic applications, wherein the presence or amount of the nucleic acid or the protein are to be assessed.

NOV21 nucleic acids and polypeptides are further useful in the generation of antibodies that bind immuno-specifically to the novel NOV21 substances for use in therapeutic or diagnostic methods. These antibodies may be generated according to methods known in the art, using prediction from hydrophobicity charts, as described in the “Anti-NOVX Antibodies” section below. The disclosed NOV21 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV22

A disclosed NOV22 nucleic acid of 1602 nucleotides (also referred to as 57724-01) encoding a carboxylesterase-like protein is shown in Table 22A. The start and stop codons are in bold letters.

TABLE 22A
NOV22 nucleotide sequence.

(SEQ ID NO:53)

ATGCGCCTGCACAGACTTCACGCGCGGCCGAGCGCGGTCGCCTGTCCGCTCCTGCTGCTTCTGATGCTGT
GTGGGCCCGAACTTCCTCAGCCTGAAGTAGACACCACCCTGGGTCGTGTGCGAGGCCGGCAGGTGGGCGT
GAAGGGCACAGACCCCCTTGTCAATGTCTTTCTGGGCATTCCATTTGCCCAGCCGCCACTGCGCCCTGAC
CGGTTCTCAGCCCCACACCCAGCACAGCCCTGGGAGGGTGTGCGGGATGCCAGCACTGCGCCCCCAATCT
GCCTACAAGACGTGATGAACAGCAGCAGATTTGTCCTCAACGGAAAACAGCAGATCTTCTCCGTTTCAGA
GGACTGCCTGGTCCTCAACGTCTATAGCCCAGCTGAGGTCATGCTATGGGTCCATGGAGGCGCTCTGATA
ACTGGCGCTGCCACCTCCTACGATGGATCAGCTCTGGCTGCCTATCGCGATGTGGTCGTGGTTACAGTCC
AGTACCGCCTTGGGGTCCTTGGCTTCTTCAGCACTGGAGATGAGCATGCACCTGGCAACCAGGGCTTCCT
AGATGTGGTAGCTGCTTTGCGCTGGGTGCAACAAAACATCGCCCCCTTCGGGGGTGACCTCAACTGTGTC
ACTGTCTTTGGTGGATCTGCCGGTGGGAGCATCATCTCTGCCCTCGTCCTGTCCCCAGTGGCTGCAGGGC
TGTTCCACAGAGCCATCACACAGAGTGGGGTCATCACCACCCCAGGGATCATCGACTCTCACCCTTGGCC
CCTAGCTCAGAAAATCGCAAACACCTTGCCCTGCAGCTCCAGCTCCCCGGCTGAGATGGTGCAGTGCCTT
CAGCAGAAAGAAGGAGAAGAGCTGGTCCTTAGCAAGAAGCTGAAAAATACTATCTATCCTCTCACCGTTG
ATGGCACTGTCTTCCCCAAAAGCCCCAAGGAACTCCTGAAGGAGAAGCCCTTCCACTCTGTGCCCTTCCT
CATGGGTGTCAACAACCATGAGTTCAGCTGGCTCATCCCCGGGACCAAGGTGATGCGTGTGTCCAACAAG
ATGATCATGAAGTTCCCGCTAAACCGGCAGGCGATGAGAAAGGAAACCATCACTAAGATGCTCTGGAGTA
CCCGCACCCTGTTGGAGCATGACTGGAAGATGCTACGAAACCGTATGATGCACATAGTTCAAGATGCCAC
TTTCGTGTATGCCACACTGCAGACTCCTCACTACCACCGAGATGCCGGCCTCCCTGTCTACCTCTATGAA
TTTGAGCACCACCCTCGTGGAATAATCGTCAAACCCCGCACTGATGGGGCAGACCATGGGGATGAGATGT
ACTTCCTCTTTGGGGGCCCCTTCGCCACAGGCCTTTCCATGGGTAAGCAGAAGGCACTTAGCCTCCAGAT
GATGAAATACTGGGCCAACTTTGCCCGCACAGGAAACCCCAATGATGGGAATCTGCCCTGCTGGCCACGC
TACAACAAGGATGAAAAGTACCTGCAGCTGGATTTTACCACAACAGTGGGCATGAAGCTCAAGGAGAAGA
AGATGGCTTTTTGGATGAGTCTGTACCAGTCTCAAAGACCTGAGAAGCAGAGGCAATTCTAA

In a search of public sequence databases, the NOV22 nucleic acid sequence, located on chromsome 16 has 695 of 735 bases (94%) identical to a gb:GENBANK-ID:AK0000105|acc:AK000105.1 mRNA from Homo sapiens (Homo sapiens cDNA FLJ20098 fis, clone COL04537, highly similar to ESTM_MOUSE LIVER CARBOXYLESTERASE PRECURSOR). Public nucleotide databases include all GenBank databases and the GeneSeq patent database.

The disclosed NOV22 polypeptide (SEQ ID NO:54) encoded by SEQ ID NO:53 has 533 amino acid residues and is presented in Table 22B using the one-letter amino acid code. Signal P, Psort and/or Hydropathy results predict that NOV22 has a signal peptide and is likely to be localized extracellularly with a certainty of 0.7953. The most likely cleavage site for a NOV22 peptide is at amino acid position 29.

TABLE 22B
Encoded NOV22 protein sequence.

| | |
|---|---|
| MRLHRLHARPSAVACGLLLLLMLCGPEVAQPEVDTTLGRVRGRQVGVKGTDRLVNVFLGI | (SEQ ID NO:54) |
| PFAQPPLGPDRFSAPHPAQPWEGVRDASTAPPMCLQDVMNSSRFVLNGKQQIFSVSEDCL | |
| VLNVYSPAEVMVWVHGGALETGAATSYDGSALAAYGDVVVVTVQYRLGVLGFFSTGDEHA | |
| PGNQGFLDVVAALRWVQENIAPFGGDLNCVTVFGGSAGGSIISGLVLSPVAAGLFHRAIT | |
| QSGVITTPGIIDSHPWPLAQKIANTLACSSSSPAEMVQCLQQKEGEELVLSKKLKNTIYP | |
| LTVDGTVFPKSPKELLKEKPFHSVPFLMGVNNHEFSWLIPGTKVMRVSNKMIMKFPLNRQ | |
| AMRKETITKMLWSTRTLLEHDWKMLRNRMMDIVQDATFVYATLQTAHYHRDAGLPVYLYE | |
| FEHHARGIIVKPRTDGADHGDEMYFLFGGPFATGLSMGKEKALSLQMMKYWANFARTGNP | |
| NDGNLPCWPRYNKDEKYLQLDFTTRVGMKLKEKKMAFWMSLYQSQRPEKQRQF | |

A search of sequence databases reveals that the NOV22 amino acid sequence has 296 of 544 amino acid residues (54%) identical to, and 373 of 544 amino acid residues (68%) similar to, the 554 amino acid residue ptnr:SWISSPROT-ACC:Q63880 protein from Mus musculus (Mouse) (LIVER CARBOXYLESTERASE PRECURSOR (EC 3.1.1.1) (ES-MALE) (ESTERASE-31))(. Public amino acid databases include the GenBank databases, SwissProt, PDB and PIR.

NOV22 is expressed in at least liver, colon, small intestine, kidney, pancrease, brain, and plasma. Expression information was derived from the tissue sources of the sequences that were included in the derivation of the sequence of CG57724-01. The sequence is predicted to be expressed in the following tissues because of the expression pattern of (GENBANK-ID: gb:GENBANK-ID:AK000105|acc:AK000105.1) a closely related Homo sapiens cDNA FLJ20098 fis, clone COL04537, highly similar to ESTM_MOUSE LIVER CARBOXYLESTERASE PRECURSOR homolog in species Homo sapiens: liver, colon, small intestine, kidney, pancrease, brain, and plasma. This information was derived by determining the tissue sources of the sequences that were included in the invention including but not limited to SeqCalling sources, Public EST sources, Literature sources, and/or RACE sources.

The disclosed NOV22 polypeptide has homology to the amino acid sequences shown in the BLASTP data listed in Table 22C.

TABLE 22C
BLAST results for NOV22

| Gene Index/ | | Length | Identity | Positives | |
|---|---|---|---|---|---|
| Identifier | Protein/Organism | (aa) | (%) | (%) | Expect |
| gi\|17512361\|gb\|AAH19147.1\| | protein for | 568 | 293/547 | 370/547 | e−156 |
| AAH19147 | MGC: 29382) [Mus | | (53%) | (67%) | |
| (BC019147) | musculus] | | | | |
| gi\|2494382\|sp\|Q63880\| | LIVER | 554 | 297/560 | 369/560 | e−155 |
| ESTM_MOUSE | CARBOXYLESTERASE | | (53%) | (65%) | |
| | PRECURSOR (ES- | | | | |
| | MALE) (ESTERASE- | | | | |
| | 31) | | | | |
| gi\|14789873\|gb\|AAH10812.1\| | (protein for | 524 | 272/538 | 346/538 | e−140 |
| AAH10812 | IMAGE: 4211034) | | (50%) | (63%) | |
| (BC010812) | [Mus musculus] | | | | |
| gi\|730714\|sp\|Q04791\| | FATTY ACYL-COA | 557 | = 252/547 | 346/547 | e−136 |
| SASB_ANAPL | HYDROLASE | | (46%) | (63%) | |
| | PRECURSOR, MEDIUM | | | | |
| | CHAIN | | | | |
| | (THIOESTERASE B) | | | | |
| gi\|57554\|emb\|CAA46391.1\| | carboxylesterase | 565 | (42%) | 336/559 | e−124 |
| (X65296) | [Rattus rattus] | | | (59%) | |

Table 22D lists the domain descriptions from DOMAIN analysis results against NOV22. This indicates that the NOV22 sequence has properties similar to those of other proteins known to contain this domain.

TABLE 22D
Domain Analysis of NOV22

gnl|Pfam|pfam00135, COesterase, Carboxylesterase
CD-Length = 532 residues, 93.0% aligned
Score = 374 bits (960), Expect = 8e−105

The mammalian carboxylesterases (EC 3.1.1.1) comprise a multigene family, the gene products of which are localized in the endoplasmic reticulum (ER) and cytosol of many tissues. These enzymes efficiently catalyze the hydrolysis of a variety of ester- and amide-containing chemicals, as well as drugs (including prodrugs) to the respective free acids. They are involved in detoxification or metabolic activation of various drugs, environmental toxicants, and carcinogens. Carboxylesterases also catalyze the hydrolysis of endogenous compounds such as short- and long-chain acylglycerols, long-chain acylcarnitine, and long-chain acyl-CoA esters. Multiple isozymes of hepatic microsomal carboxylesterases exist in various animal species, and some of these isozymes are involved in the metabolic activation of certain carcinogens and are associated with hepatocarcinogenesis.

Several studies have shown that various carboxylesterases are present in a wide variety of organs and tissues of many mammalian species; the highest hydrolase activity occurs in the liver. Humans express carboxylesterase in the liver, small intestine, brain, stomach, colon, pancreas, kidney, macrophages, monocytes, and plasma. Carboxylesterases, in addition to the metabolism of exogenous compounds, have been shown to hydrolyze endogenous fatty acid esters of steroids in both rat pancreas and kidney. The nonspecific esterases found in brain appear to be present only in the central nervous system, and four unique carboxylesterases have been isolated from human brain extract. Carboxylesterase activity of is found predominantly in the microsomal fraction, although significant carboxylesterase activity is present in the lysosomal fraction, and the lysosomes contribute substantially to the general esterolytic capacity of liver. The microsomal and lysosomal enzymes can be differentiated on the basis of both substrate specificity and structure and are considered to belong to separate classes. Carboxylesterase activity is also found in the cytosolic fraction of brain and in the plasma. Carboxylesterase is present in the plasma, but it is most likely syn-thesized in liver and then secreted into the circulation via the Golgi apparatus.

Human liver and plasma carboxylesterase activates lovastatin, and there are a significant number of additional drugs and endogenous compounds that are substrates of carboxylesterases, e.g. dipivefrin hydrochloride, carbonates, cocaine, salicylates, capsaicin, palmitoyl-coenzyme A, haloperidol, imidapril, pyrrolizidine alkaloids, and steroids.

The disclosed NOV22 nucleic acid of the invention encoding a carboxylesterase-like protein includes the nucleic acid whose sequence is provided in Table 22A or a fragment thereof. The invention also includes a mutant or variant nucleic acid any of whose bases may be changed from the corresponding base shown in Table 22A while still encoding a protein that maintains its carboxylesterase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 6 percent of the bases may be so changed.

The disclosed NOV22 protein of the invention includes the carboxylesterase-like protein whose sequence is provided in Table 22B. The invention also includes a mutant or variant protein any of whose residues may be changed from the corresponding residue shown in Table 22B while still encoding a protein that maintains its carboxylesterase-like activities and physiological functions, or a functional fragment thereof. In the mutant or variant protein, up to about 46 percent of the residues may be so changed.

The invention further encompasses antibodies and antibody fragments, such as F_{ab} or (F_{ab})₂, that bind immunospecifically to any of the proteins of the invention.

The above defined information for this invention suggests that this carboxylesterase-like protein (NOV22) may function as a member of a “carboxylesterase family”. Therefore, the NOV22 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV22 nucleic acids and proteins of the invention are useful in potential therapeutic applications implicated in diseases including but not limited to various pathologies and disorders as indicated below. For example, a cDNA encoding the carboxylesterase-like protein (NOV22) may be useful in gene therapy, and the carboxylesterase-like protein (NOV22) may be useful when administered to a subject in need thereof. By way of nonlimiting example, the compositions of the present invention will have efficacy for treatment of patients suffering from hepatocarcinoma, as well as other diseases, disorders and conditions, along with patients receiving pharmacotherapy with drug classes known to be metabolized by carboxylesterases, such as salicylates, carbonates, pyrrolizidine alkaloids, and steroids, or other pathologies or conditions. The NOV22 nucleic acid encoding the carboxylesterase-like protein of the invention, or fragments thereof, may further be useful in diagnostic applications, wherein the presence or amount of the nucleic acid or the protein are to be assessed.

NOV22 nucleic acids and polypeptides are further useful in the generation of antibodies that bind immuno-specifically to the novel NOV22 substances for use in therapeutic or diagnostic methods. These antibodies may be generated according to methods known in the art, using prediction from hydrophobicity charts, as described in the “Anti-NOVX Antibodies” section below. The disclosed NOV22 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV23

A disclosed NOV23 nucleic acid of 996 nucleotides (also referred to as CG57730-01) encoding a MAT-1-like protein is shown in Table 23A. The start and stop codons are in bold letters.

TABLE 23A
NOV23 nucleotide sequence.

| | |
|---|---|
| CTCTCGAATTCCCCCACCCACCTGTACTCTGGAGAGACTGTGCTGGGAACATGTACCACT | (SEQ ID NO:55) |
| GAGCCTGAGATGGGGATGAGGGCAGAGAGAGGGGAGCCCCCTCTTCCACTCAGTTGTTCC | |
| TACTCAGACTGTTGCACTCTAAACCTAGGGAGGTTGAAGAATGAGACCCTTAGGTTTTAA | |
| CACGAATCCTGACACCACCATCTATAGGGTCCCAACTTCGTTATTGTAGGCAACCTTCCC | |
| TCTCTCCTTGGTGAAGAACATCCCAAGCCAGAAAGAAGTTAACTACAGTGTTTTCCTTTG | |
| CACCGATCCCCACCCCAATTCAATCCCGGAAGGGACTTACTTAGGAAACCCTTCTTTACT | |
| AGATATCCTGGCCCCCTGGGCTTGTGAACACCTCCTAGCCACATCACTACAGTACAGTGA | |
| GTGACCCCAGCCTCCTGCCTACCCCAAGATGCCCCTCCCCACCCTGACCGTGCTAACTGT | |
| GTGTACATATATATTCTACATATATGTATATTAAAACTGCACTGCCATGTCTGCCCTTTT | |
| TTGTGGTGTCTAGCATTAACTTATTGTCTAGGCCAGAGCGGGGGTGGGAGGGGAATGCCA | |
| CAGTGAAGGGAGTGGCAGAATCAAATTGCTACATAGTCCAAACAAAAAAGAAGGCTTTTT | |
| CAAAAAACATTAAATTCACATGCAGTCTCAGAGACTATTTAGACAAAGTTCAAGTTAGGA | |
| GCTTTTAGGATGTGGGAGTAAAACTTTAATGGGAGGGGAGGGCTGGCTGCTGGAAGAAGG | |
| AAGAAGCCAGACTGGTTAGACAGTACTCTTAACTCCTAGCCCAGCCTAGCGTGCCCTGCC | |
| CCTCTGGCCACTGCTGCAGACACCTGCCTTAACACACACACCTCTAGGACTCCACAGTTT | |
| TGCCTTAAACGACCTTCCCAAGTCTCCCTTTCCCTGTCTGGCTTCTCCCTTAAGAAGAGA | |
| GAGATACTTGTAGAATTGGGTGGGGGAATTCGAGAG | |

In a search of public sequence databases, the NOV23 nucleic acid sequence, located on chromsome 1q21.1 has 983 of 987 bases (99%) identical to a gb:GENBANK-ID:PEAGENE3|acc:AF153274.1 mRNA from Homo sapiens (Homo sapiens PEA15 protein (PEA15) gene, exons 3 and 4 and complete cds). Public nucleotide databases include all GenBank databases and the GeneSeq patent database.

The disclosed NOV23 polypeptide (SEQ ID NO:56) encoded by SEQ ID NO:55 has 74 amino acid residues and is presented in Table B using the one-letter amino acid code. Signal P, Psort and/or Hydropathy results predict that NOV23 has a signal peptide and is likely to be localized in the endoplasmic reticulum with a certainty of 0.5500. The most likely cleavage site for a NOV23 peptide is between amino acids 18 and 19.

TABLE 23B
Encoded NOV23 protein sequence.

| | |
|---|---|
| MYIKTALPCLPFFVVSSINLLSRPERGWEGNATVKGVAESNCYIVQTKKKAFSKNIKFTC | (SEQ ID NO:56) |
| SLRDYLDKVQVRSF | |

A search of sequence databases reveals that the NOV23 amino acid sequence has 62 of 75 amino acid residues (82%) identical to, and 66 of 75 amino acid residues (88%) similar to, the 75 amino acid residue ptnr:SPTREMBL-ACC:Q14801 protein from Homo sapiens (human) (HYPOTHETICAL 8.6 KDA PROTEIN). Public amino acid databases include the GenBank databases, SwissProt, PDB and PIR.

NOV23 is expressed in at least ovary, testis, brain, amygdala, pancreas, colon, and stomach. This information was derived by determining the tissue sources of the sequences that were included in the invention including but not limited to SeqCalling sources, Public EST sources, Literature sources, and/or RACE sources.

The disclosed NOV23 polypeptide has homology to the amino acid sequences shown in the BLASTP data listed in Table 23C.

TABLE 23C
BLAST results for NOV23

| Gene Index/ | | Length | Identity | Positives | |
|---|---|---|---|---|---|
| Identifier | Protein/Organism | (aa) | (%) | (%) | Expect |
| gi\|14714659\|gb\|AAH10469.1\| | Similar to | 74 | 74/74 | 74/74 | 1e−34 |
| AAH10469 | homolog of mouse | | (100%) | (100%) | |
| (BC010469) | MAT-1 oncogene | | | | |
| | [Homo sapiens] | | | | |
| gi\|7019425\|ref\|NP_037419.1\| | homolog of mouse | 75 | 62/75 | 66/75 | 5e−27 |
| (NM_013287) | MAT-1 oncogene; | | (82%) | (87%), | |
| | Phosphoprotein | | | | |
| | enriched in | | | | |
| | astrocytes, 15 kD | | | | |
| | [Homo sapiens] | | | | |
| gi\|6678812\|ref\|NP_032582.1\| | phosphoprotein | 61 | 27/30 | 27/30 | 8e−9 |
| (NM_008556) | enriched in | | (90%) | (90%) | |
| | astrocytes 15; | | | | |
| | mammary | | | | |
| | transforming gene | | | | |
| | 1 [Mus musculus] | | | | |

An efficient in vitro transformation system has been developed using N-methyl-N-nitrosourea that allows the role of hormones and growth factors in mouse mammary tumorigenesis to be studied. Utilizing this system, it was reported that mammary tumors induced in vitro with N-methyl-N-nitrosourea in the presence of mammogenic hormones (progesterone and prolactin) contain predominately an activated c-Ki-ras protooncogene with a G35 [symbol] A35 transitional mutation in the 12th codon. Mammary tumors induced in the presence of another mitogen, lithium (Li), do not have a mutation in the c-Ki-ras protooncogene. By using an expression cloning system, a plasmid clone containing a 1.75-kb cDNA insert has been isolated from this group of tumors. Nucleic acid sequence analysis of the insert reveals that it has a short open reading frame of 61 amino acids and that it does not have sequence homology with any known gene. The gene, designated MAT1, can neoplastically transform NIH 3T3 cells and also the mammary epithelial cell line TM3. Expression of this gene occurs in normal mouse tissues including mammary gland and is overexpressed in the original mammary tumors as indicated by Northern blot analysis. In vitro transcription and translation of the clone shows a protein product of 6000 Da, which agrees with the predicted open reading frame.

The disclosed NOV23 nucleic acid of the invention encoding a MAT-1-like protein includes the nucleic acid whose sequence is provided in Table 23A or a fragment thereof. The invention also includes a mutant or variant nucleic acid any of whose bases may be changed from the corresponding base shown in Table 23A while still encoding a protein that maintains its MAT-1-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV23 protein of the invention includes the MAT-1-like protein whose sequence is provided in Table 23B. The invention also includes a mutant or variant protein any of whose residues may be changed from the corresponding residue shown in Table 23B while still encoding a protein that maintains its MAT-1-like activities and physiological functions, or a functional fragment thereof. In the mutant or variant protein, up to about 12 percent of the residues may be so changed.

The invention further encompasses antibodies and antibody fragments, such as F_{ab} or (F_{ab})₂, that bind immunospecifically to any of the proteins of the invention.

The above defined information for this invention suggests that this MAT-1-like protein (NOV23) may function as a member of a “MAT-I family”. Therefore, the NOV23 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV23 nucleic acids and proteins of the invention are useful in potential therapeutic applications implicated in diseases including but not limited to various pathologies and disorders as indicated below. For example, a cDNA encoding the MAT-1-like protein (NOV23) may be useful in gene therapy, and the MAT-1-like protein (NOV23) may be useful when administered to a subject in need thereof. By way of nonlimiting example, the compositions of the present invention will have efficacy for treatment of patients suffering from Cataract, zonular pulverulent-1; MHC class II deficiency, complementation group C; cancer, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration; diabetes, pancreatitis, obesity; fertility, or other pathologies or conditions. The NOV23 nucleic acid encoding the MAT-1-like protein of the invention, or fragments thereof, may further be useful in diagnostic applications, wherein the presence or amount of the nucleic acid or the protein are to be assessed.

NOV23 nucleic acids and polypeptides are further useful in the generation of antibodies that bind immuno-specifically to the novel NOV23 substances for use in therapeutic or diagnostic methods. These antibodies may be generated according to methods known in the art, using prediction from hydrophobicity charts, as described in the “Anti-NOVX Antibodies” section below. The disclosed NOV23 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV24

A disclosed NOV24 nucleic acid of 668 nucleotides (also referred to as CG57755-01) encoding a vacuolar proton-ATPase subunit H-like protein is shown in Table 24A. The start and stop codons are in bold letters.

TABLE 24A
+HZ,/47
NOV24 nucleotide sequence.

| | |
|---|---|
| TTCGCCCTCCCGGTCATCATCTTCACCACGTTCTGGGGCCTCGTCGGCATCGCCGGGCCC | (SEQ ID NO:57) |
| TGGTTCGTGCCGAAGGGACCCAACCGCGGAGTGATCATCACCATGCTGGTCGCCACCGCC | |
| GTCTGCTGTTACCTCTTCTGGCTCATCGCCATCCTGGCGCAGCTGAACCCCCTGTTCGGG | |
| CCCCAGCTGAAGAATGAGACCATCTGGTACGTGCGCTTCCTGTGGGAGTGACCCGCCGCC | |
| CCCGACCCAGGTGCCCAGCTCTCGGAATGACTGTGGCTCCACTGTCCCTGACAACCCCTT | |
| CGTCCGGACCCTCCCCCACACAACTATGTCTGGTCACCAGCTCCCTCCTGCTGGCACCCA | |
| GAGACCCGGACCCGCAGGGCCTGCCTGGTTCCTGGAAGTCTTCCCAGTCTTCCCAGCCAG | |
| CCCGGGCCCTGGGGAGCCCTGGGCACAGCAGCGGCCGAGGGGATGTCCTGCTCCAATACC | |
| CGCACTGCTCTGGAGTTTGCCCTCTTTCCCAAGGAGATGCTGCTGGGGAGCTGGTATGGG | |
| TGGGGTCTTTCCCTTTACAGACGGGGCAGATGCCAGGACTCAGCCCATCCTCAGGAGGAC | |
| ACGTGTCCTCATGGAGAGGGTGCTCCGGCCCAGGCGGGGGAGTCGGTGCCCAGTCAGCAG | |
| GACCAGGC | |

In a search of public sequence databases, the NOV24 nucleic acid sequence has 169 of 230 bases (73%) identical to a gb:GENBANK-ID:AF258614|acc:AF258614.1 mRNA from Canis familiaris (Canis familiaris vacuolar proton-ATPase subunit ATP6H (ATP6H) mRNA, complete cds). Public nucleotide databases include all GenBank databases and the GeneSeq patent database.

The disclosed NOV24 polypeptide (SEQ ID NO:58) encoded by SEQ ID NO:57 has 76 amino acid residues and is presented in Table 24B using the one-letter amino acid code. Signal P, Psort and/or Hydropathy results predict that NOV24 has a signal peptide and is likely to be localized in the plasma membrane with a certainty of 06400. The most likely cleavage site for a NOV24 peptide is between amino acids 53 and 54.

TABLE 24B
Encoded NOV24 protein sequence.

| | |
|---|---|
| FALPVIIFTTFWGLVGIAGPWFVPKGPNRGVIITMLVATAVCCYLFWLIAILAQLNPLFG | (SEQ ID NO:58) |
| PQLKNETIWYVRFLWE | |

A search of sequence databases reveals that the NOV24 amino acid sequence has 56 of 73 amino acid residues (76%) identical to, and 64 of 73 amino acid residues (87%) similar to, the 81 amino acid residue ptnr:SPTREMBL-ACC:Q9N0Q1 protein from Canis familiaris (Dog) (VACUOLAR PROTON-ATPASE SUBUNIT ATP6H). Public amino acid databases include the GenBank databases, SwissProt, PDB and PIR.

The disclosed NOV24 polypeptide has homology to the amino acid sequences shown in the BLASTP data listed in Table 24C.

TABLE 24C
BLAST results for NOV24

| Gene Index/ | | Length | Identity | Positives | |
|---|---|---|---|---|---|
| Identifier | Protein/Organism | (aa) | (%) | (%) | Expect |
| gi\|14789787\|gb\|AAH10790.1\| | protein for | 81 | 75/76 | 76/76 | 4e−28 |
| AAH10790 | MGC: 18845) [Mus | | (98%) | (99%) | |
| (BC010790) | musculus] | | | | |
| gi\|18600961\|ref\|XP_ | protein XP_095170 | 109 | 65/86 | 67/86 | 9e−22 |
| 095170.1\| | [Homo sapiens] | | (75%) | (77%) | |
| (XM_095170) | | | | | |
| gi\|8050819\|gb\|AAF71753.1\| | vacuolar proton- | 81 | 56/73 | 64/73 | 3e−21 |
| AF258614_1 | ATPase subunit | | (76%) | (86%) | |
| (AF258614) | ATP6H [Canis | | | | |
| | familiaris] | | | | |
| gi\|13507387\|gb\|AAK28556.1\| | lysosomal H+ | 81 | 55/73 | 64/73 | 6e−21 |
| AF343440_1 | transporting- | | (75%) | (87%) | |
| (AF343440) | ATPase subunit | | | | |
| | M9.2 [Canis | | | | |
| | familiaris] | | | | |
| gi\|13384606\|ref\|NP_ | ATPase, H+ | 81 | 55/73 | 65/73 | 8e−21 |
| 079548.1\| | transporting | | (75%) | (88%) | |
| (NM_025272) | lysosomal | | | | |
| | (vacuolar proton | | | | |
| | pump), 9.2 kDa | | | | |
| | [Mus musculus] | | | | |

Vacuolar-type H(+)-ATPase (V-ATPase) is a multisubunit enzyme responsible for acidification of eukaryotic intracellular organelles. V-ATPase-dependent organelle acidification is essential for intracellular processes such as protein sorting, zymogen activation, and receptor-mediated endocytosis. The V-ATPase is composed of peripheral (V1) and integral (V0) membrane sectors. Proteolipids are major components of the V0 sector. In C. elegans, Oka et al. (See Oka, T. et al., J. Biol. Chem. 272: 24387-24392, 1997) identified the VHA1 and VHA2 genes, which encode 16-kD proteolipids, and the VHA4 gene, which encodes a 23-kD proteolipid product. Nishigori et al. (1998) isolated human cDNAs encoding a proteolipid which they designated ATP6F. Sequence analysis revealed that the predicted 205-amino acid protein shares 61% identity with the S. cerevisiae proteolipid VMA16 and 67% identity with C. elegans VHA4. ATP6F contains 5 transmembrane segments and a conserved glutamic acid residue that is essential for proton transport activity in VMA16. As with ATP6C (108745), a 16-kD V-ATPase proteolipid, the N- and C-terminal halves of ATP6F share homology and may have resulted from a gene duplication event. The duplicated segments of ATP6F and ATP6C are 75% similar on the amino acid level. Northern blot analysis indicated that the 1.1-kb ATP6F mRNA was expressed in all tissues tested. The ATP6F gene contains 8 exons and spans approximately 4 kb. By FISH and radiation hybrid analysis, Nishigori et al. (1998) mapped the ATP6F gene to 1p32.3. (See Nishigori et al., Genomics 50: 222-228, 1998).

The vacuolar proton-ATPase (V-ATPase) is composed of an extramembrane catalytic sector and a transmembrane proton-conducting sector. See 603717. Ludwig et al. (1998) identified 2 novel proteins, 8-9 and 9.2 kD in size, in the membrane sector of bovine chromaffin granule V-ATPase. They designated the larger protein M9.2. By searching an EST database with the N-terminal sequence of bovine M9.2, Ludwig et al. (See Ludwig, et al., J. Biol. Chem. 273: 10939-10947, 1998) identified homologous cDNAs from human and mouse. The deduced 80-amino acid human M9.2 protein is extremely hydrophobic with 2 predicted membrane-spanning helices. Human and mouse M9.2 differed at only 1 amino acid position. Northern blot analysis revealed that M9.2 was present in all bovine tissues tested.

The disclosed NOV24 nucleic acid of the invention encoding a vacuolar proton-ATPase subunit H-like protein includes the nucleic acid whose sequence is provided in Table 24A or a fragment thereof. The invention also includes a mutant or variant nucleic acid any of whose bases may be changed from the corresponding base shown in Table 24A while still encoding a protein that maintains its vacuolar proton-ATPase subunit H-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 27 percent of the bases may be so changed.

The disclosed NOV24 protein of the invention includes the vacuolar proton-ATPase subunit H-like protein whose sequence is provided in Table 24B. The invention also includes a mutant or variant protein any of whose residues may be changed from the corresponding residue shown in Table 24B while still encoding a protein that maintains its vacuolar proton-ATPase subunit H-like activities and physiological functions, or a functional fragment thereof.

In the mutant or variant protein, up to about 24 percent of the residues may be so changed.

The invention further encompasses antibodies and antibody fragments, such as F_{ab} or (F_{ab})₂, that bind immunospecifically to any of the proteins of the invention.

The above defined information for this invention suggests that this vacuolar proton-ATPase subunit H-like protein (NOV24) may function as a member of a “vacuolar proton-ATPase subunit H family”. Therefore, the NOV24 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV24 nucleic acids and proteins of the invention are useful in potential therapeutic applications implicated in diseases including but not limited to various pathologies and disorders as indicated below. For example, a cDNA encoding the vacuolar proton-ATPase subunit H-like protein (NOV24) may be useful in gene therapy, and the vacuolar proton-ATPase subunit H-like protein (NOV24) may be useful when administered to a subject in need thereof. By way of nonlimiting example, the compositions of the present invention will have efficacy for treatment of patients suffering from polycystic kidney disease I; osteopetrosis; mucolipidosis IV, or other pathologies or conditions. The NOV24 nucleic acid encoding the vacuolar proton-ATPase subunit H-like protein of the invention, or fragments thereof, may further be useful in diagnostic applications, wherein the presence or amount of the nucleic acid or the protein are to be assessed.

NOV24 nucleic acids and polypeptides are further useful in the generation of antibodies that bind immuno-specifically to the novel NOV24 substances for use in therapeutic or diagnostic methods. These antibodies may be generated according to methods known in the art, using prediction from hydrophobicity charts, as described in the “Anti-NOVX Antibodies” section below. The disclosed NOV24 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV25

A disclosed NOV25 nucleic acid of 5587 nucleotides (also referred to as CG57503-01) encoding a MEGF7-like protein is shown in Table 25A. The start and stop codons are in bold letters.

TABLE 25A
NOV25 nucleotide sequence.

(SEQ ID NO:59)

ATGGGCCTAGCAGTCATACTACCTACCTGTTCCCCTCTTGACTTTCACTGTGACAATGGCAAGTGCATCC
GCCGCTCCTGGGTGTGTGACGGGGACAACGACTGTGAGGATGACTCCGATCAGCAGGACTGTCCCCCCCG
GGAGTGTGAGGAGGACGAGTTTCCCTGCCAGAATGGCTACTGCATCCGGAGTCTGTCGCACTGCCATGGT
GACAATGACTGTGGCGACAACAGCGATGAGCAGTGTGACATGCGCAAGTGCTCCGACAAGGAGTTCCCCT
GTAGTGACGGAAGCTGCATTGCTGAGCATTGGTACTGCCACGGTGACACCGACTGCAAAGATGGCTCCGA
TGAGGAGAACTGTCCCTCAGCAGTGCCAGCGCCCCCCTGCAACCTGGAGGAGTTCCAGTGTGCCTATGGA
CGCTGCATCCTCGACATCTACCACTGCGATGGCGACGATGACTGTGGACACTGGTCAGACGAGTCTGACT
GCTGTGAGTACTCTGGCCAGCTGGGAGCCTCCCACCAGCCCTGCCGCTCTGGGGAGTTCATGTGTGACAG
TGGCCTGTGCATCAATGCAGGCTGGCGCTGCGATGGTGACGCGGACTGTGATGACCAGTCTGATGAGCGC
AACTGCACCACCTCCATGTGTACGGCAGAACACTTCCGCTGTCACTCAGGCCGCTGTGTCCGCCTGTCCT
GGCGCTGTGATGGGGAGGACGACTGTGCAGACAACACCGATGAAGAGAACTGTGAGAATACAGGAAGCCC
CCAATGTGCCTTGGACCAGTTCCTGTGTTGGAATGGGCGCTGCATTGGGCAGAGGAAGCTGTGCAACGGG
GTCAACGACTGTGGTGACAACAGCGACGAAAGCCCACAGCAGAATTGCCGGCCCCGGACGGGTGAGGAGA
ACTGCAATGTTAACAACCGTGGCTGTGCCCAGAAGTGCCAGATGGTCCGGCGGGCAGTGCAGTGTACCTG
CCACACAGGCTACCGGCTCACAGAGGATGGGCACACGTGCCAAGATGTGAATGAATGTCCCGAGGAGGGG
TATTGCAGCCAGGGCTGCACCAACACCGAAGGGGCTTTCCAATGCTGGTGTGAAACAGGCTATGAACTAC
GGCCCGACCGGCGCAGCTGCAAGGCTCTCGGGCCAGAGCCTGTGCTGCTGTTCGCCAATCGCATCGACAT
CCGGCAGGTGCTGCCACACCGCTCTGAGTACACACTGCTGCTTAACAACCTGGAGAATGCCATTGCCCTT
GATTTCCACCACCGCCGCGAGCTTGTCTTCTGGTCAGATGTCACCCTGGACCGGATCCTCCGTGCCAACC
TCAACGGCAGCAACGTGGAGGAGGTTGTGTCTACTGCCCTGGAGAGCCCAGGGGGCCTGGCTGTGGATTG
GGTCCATCACAAACTCTACTGGACCGACTCAGGCACCTCGACGATTGAGGTGGCCAATCTGGACGGGGCC
CACCGGAAAGTGTTGCTGTGGCAGAACCTGGAGAAGCCCCGGGCCATTGCCTTGCATCCCATGGAGGGTA
CCATTTACTGGACAGACTGGGGCAACACCCCCCGTATTGAGGCCTCCAGCATGGATGGCTCTGGACGCCG
CATCATTGCCGATACCCATCTCTTCTCGCCCAATGGCCTCACCATCGACTATGCCCGGCGCCGTATGTAC
TGGGTGGATGCTAAGCACCATGTCATCGAGAGGGCCAATCTGGATGGGAGTCACCGTAAGGCTGTCATTA
GCCAGGGCCTCCCGCATCCCTTCGCCATCACAGTCTTTGAAGACAGCCTGTACTGGACAGACTGGCACAC
CAACAGCATCAATAGCGCTAACAAATTTACGGGGAACAACCAGGAAATCATTCGCAACAAACTCCACTTC
CCTATCGACATCCACACCTTGCACCCCCAGCGCCAACCTGCAGGGAAAAACCGCTGTGGGGACAACAACG
GAGGCTGCACGCACCTCTGTCTGCCCAGTGGCCAGAACTACACCTGTGCCTCCCCCACTGGCTTCCGCAA
GATCAGCAGCCACGCCTGTGCCCAGAGTCTTGACAAGTTCCTGCTTTTTGCCCGAACCATCGACATCCGT
CGAATCAGCTTTGACACAGAGCACCTGTCTGATGATGTCATCCCACTGGCTGACCTGCCCAGTGCTGTGG
CCCTTGACTGGGACTCCCGGGATGACCACGTGTACTGGACAGATGTCAGCACTGATACCATCACCAGCGC
CAAGTGGGATGGAACAGGACAGGAGGTGGTAGTCCATACCAGTTTGGAGAGCCCAGCTGGCCTGGCCATT
GATTGCGTCACCAACAAACTGTACTGGACAGATGCAGGTACAGACCGGATTGAAGTAGCCAACACAGATG
GCAGCATGAGAACAGTACTCATCTGGGAGAACCTTGATCGTCCTCGGCACATCGTGGTGGAACCCATGGG
CGGGTACATGTATTGGACTGACTGGGGTGCGAGCCCCAAGATTGAACGAGCTGGCATGGATGCCTCAGGC
CGCCAAGTCATTATCTCTTCTAATCTCACCTGCCCTAATGGGTTAGCTATTGATTATGGGTCCCACCGTC
TATACTGGGCTGACGCCGGCATGAAGACAATTGAATTTGCTGGACTGGATGGCAGTAAGAGGAAGGTGCT
GATTGGAAGCCACCTCCCCCACCCATTTGGGCTGACCCTCTATGGAGACCGCATCTATTGGACTGACTGG
CAGACCAAGAGCATACAGAGCGCTGACCGGCTGACAGGGCTGGACCGGGAGACTCTCCAGGAGAACCTGG
AAAACCTAATGGACATCCATGTCTTCCACCGCCGCCGGCCCCCAGTGTCTACACCATGTGCTATGGACAA
TGCCCGCTGTAGCCACCTGTGTCTTAGGTCCCCAAATCCAAGCCGATTCAGCTGTACCTGCCCCACAGGC
ATCAACCTGCTGTCTGATGGCAAGACCTGCTCACCAGGCATGAACAGTTTCCTCATCTTCGCCAGGAGGA
TAGACATTCGCATGGTCTCCCTGGACATCCCTTATTTTGCTGATGTGGTGGTACCAATCAACATTACCAT
GAAGAACACCATTGCCATTGGAGTAGACCCCCAGGAAGGAAACGTGTACTGGTCTGACAGCACACTGCAC
AGGATCAGTCGTGCCAATCTGGATGGCTCACAGCATGAGGACATCATCACCACAGGGCTACAGACCACAG
ATGGGCTCGCGGTTCATGCCATTCCCCGGAAAGTATACTGGACAGACACGGGAACAAACCGGATTGAAGT
GGGCAACCTGGACGCGTCCATGCGGAAAGTGTTGGTGTGGCAGAACCTTGACAGTCCCCGGGCCATCGTA
CTGTACCATGAGATGGGGTTTATGTACTGGACAGACTGGGGGGAGAATGCCAAGTTAGAGCGGTCCGGAA
TGGATGGCTCAGACCGCGCGGTGCTCATCAACAACAACCTAGGATGGCCCAATGCACTGACTGTGGACAA
GGCCAGCTCCCAACTGCTATGGGCCGATGCCCACACCGAGCGAATTGAGGCTGCTGACCTGAATGGTGCC
AATCGGCATACATTGGTGTCACCCGTGCAGCACCCATATGGCCTCACCCTGCTCGACTCCTATATCTACT
GGACTGACTGGCAGACTCGGAGCATCCACCGTGCTGACAAGGGTACTGGCAGCAATGTCATCCTCGTGAG
GTCCAACCTGCCAGGCCTCATGGACATCCAGGCTGTGGACCGGGCACAGCCACTAGGTTTTAACAAGTGC
GGCTCGAGAAATGGCGGCTGCTCCCACCTCTCCTTCCCTCGGCCTTCTGGCTTCTCCTGTGCCTGCCCCA
CTGCCATCCAGCTGAACGGAGATGGGAACACCTGTCATCCCTCTCCTGAGACCTACCTGCTCTTCTCCAG
CCGTGGCTCCATCCCCCGTATCTCACTGGACACCAGTCACCACACCAATCTCCATGTCCCTGTTCCTGAG
CTCAACAATGTCATCTCCCTGGACTATGACAGCGTGGATGGAAAGCTCTATTACACACATGTCTTCCTGG
ATGTTATCAGGCGAGCAGACCTGAACGGCAGCAACATGGAGACAGTGATCGGGCGAGGGCTGAACACCAC
TGACGGGCTGGCAGTGGACTGGGTGGCCAGGAACCTGTACTGGACAGACACAGGTCGAAATACCATTCAG
GCGTCCAGGCTGGATGGTTCCTGCCGCAAAGTACTGATCAACAATAGCCTGGATGAGCCCCGGGCCATTG
CTGTTTTCCCCAGGAAGCGGTACCTCTTCTGGACAGACTGGGGCCACATTGCCAAGATCGAACGGGCAAA
CTTGGATGGTTCTGAGCGGAAGGTCCTCATCAACACAGACCTGGGTTGGCCCAATGGCCTTACCCTGGAC
TATGATACCCGCAGGATCTACTGGGTGGATGCGCATCTGGACCGGATCGAGAGTGCTGACCTCAATGGGA
AACTGCGGCAGGTCTTGGTCAGCCATGTGTCCCACCCCTTTGCCCTCACACAGCAAGACAGGTGGATCTA
CTGGACAGACTGGCAGACCAAGTCAATCCAGCGTGTTGACAAATACTCAGGCCGGAACAAGGAGACAGTG
CTGGCAAATGTGGAAGGACTCATGGATATCATCGTGGTTTCCCCTCAGCGGCAGACAGGGACCAATGCCT
GTGGTGTGAACAATGGTGCCTCCACCCACCTCTGCTTTGCCAGAGCCTCGGACTTCGTATGTGCCTGTCC
TGACGAACCTGATAGCCAGCCCTGCTCCCTTGTGCCTGGCCTGGTACCACCAGCTCCTAGGGCTACTCCC
ATGAGTGAAAAGAGCCCAGTGCTACCCAACACACCACCTACCACCTTGTATTCTTCAACCACCCGGACCC
GCACGTCTCTGGAGGAGGTGGAAGGAAGATGCTCTGAAAGGGATGCCAGGCTGGGCCTCTGTCCACGTTC
CAATGACGCTGTTCCTGCTGCTCCAGGGGAAGGACTTCATATCAGCTACGCCATTGGTGGACTCCTCAGT
ATTCTGCTGATTTTGGTCGTGATTGCAGCTTTGATGCTGTACAGACACAAAAAATCCAAGTTCACTGATC
CTGGAATGGGGAACCTCACCTACAGCAACCCCTCCTACCGAACATCCACACAGGAAGTGAAGATTGAAGC
AATCCCCAAACCAGCCATGTACAACCAGCTGTGCTATAAGAAAGAGGCACGGCCTGACCATAACTACACC
AAGGAGAAGATCAAGATCGTAGAGGGAATCTGCCTCCTGTCTGGGGATGATGCTGAGTGGGATGACCTCA
AGCAACTGCGAAGCTCACGGGGGGGCCTCCTCCGGGATCATGTATGCATGAAGACAGACACGGTGTCCAT
CCAGGCCAGCTCTCGCTCCCTCGATGACACAGAGACGGAGCAGCTGTTACAGGAAGAGCAGTCTGAGTGT
AGCAGCGTCCATACTCCAGCCACTCCACAAAGACGAGGCTCTCTGCCAGACACGGGCTGGAAACATGAAC
GCAAGCTCTCCTCAGAGAGCCAGGTCTAAATCCCCACATTCTCTTCCCTGCCTCCCT

In a search of public sequence databases, the NOV25 nucleic acid sequence, located on chromsome 11 has 4754 of 4759 bases (99%) identical to a gb:GENBANK-ID:AB01540|acc:AB011540.1 mRNA from Homo sapiens (Homo sapiens mRNA for MEGF7, partial cds). Public nucleotide databases include all GenBank databases and the GeneSeq patent database.

The disclosed NOV25 polypeptide (SEQ ID NO:60) encoded by SEQ ID NO:59 has 1852 amino acid residues and is presented in Table 25B using the one-letter amino acid code. Signal P, Psort and/or Hydropathy results predict that NOV25 has no signal peptide and is likely to be localized at the plasma membrane with a certainty of 0.8200.

TABLE 25B
Encoded NOV25 protein sequence.

| | |
|---|---|
| MGLGVILPTCSPLDFHCDNGKCIRRSWVCDGDNDCEDDSDEQDCPPRECEEDEFPCQNGY | (SEQ ID NO:60) |
| CIRSLWHCDGDNDCGDNSDEQCDMRKCSDKEFRCSDGSCIAEHWYCDGDTDCKDGSDEEN | |
| CPSAVPAPPCNLEEFQCAYGRCILDIYHCDGDDDCGDWSDESDCCEYSGQLGASHQPCRS | |
| GEFMCDSGLCINAGWRCDGDADCDDQSDERNCTTSMCTAEQFRCHSGRCVRLSWRCDGED | |
| DCADNSDEENCENTGSPQCALDQFLCWNGRCIGQRKLCNGVNDCGDNSDESPQQNCRPRT | |
| GEENCNVNNGGCAQKCQMVRGAVQCTCHTGYRLTEDGHTCQDVNECAEEGYCSQGCTNSE | |
| GAFQCWCETGYELRPDRRSCKALGPEPVLLFANRIDIRQVLPHRSEYTLLLNNLENAIAL | |
| DFHHRRELVFWSDVTLDRILRANLNGSNVEEVVSTGLESPGGLAVDWVHDKLYWTDSGTS | |
| RIEVANLDGAHRKVLLWQNLEKPRAIALHPMEGTIYWTDWGNTPRIEASSMDGSGRRIIA | |
| DTHLFWPNGLTIDYAGRRMYWVDAKHHVIERANLDGSHRKAVISQGLPHPFAITVFEDSL | |
| YWTDWHTKSINSANKFTGKNQEIIRNKLHFPMDIHTLHPQRQPAGKNRCGDNNGGCTHLC | |
| LPSGQNYTCACPTGFRKISSHACAQSLDKFLLFARRMDIRRISFDTEDLSDDVIPLADVR | |
| SAVALDWDSRDDHVYWTDVSTDTISRAKWDGTGQEVVVDTSLESPAGLAIDWVTNKLYWT | |
| DAGTDRIEVANTDGSMRTVLIWENLDRPRDIVVEPMGGYMYWTDWGASPKIERAGMDASG | |
| RQVIISSNLTWPNGLAIDYGSQRLYWADAGMKTIEFAGLDGSKRKVLIGSQLPHPFGLTL | |
| YGERIYWTDWQTKSIQSADRLTGLDRETLQENLENLMDIHVFHRRRPPVSTPCAMENGGC | |
| SHLCLRSPNPSGFSCTCPTGINLLSDGKTCSPGMNSFLIFARRIDIRMVSLDIPYFADVV | |
| VPINITMKNTIAIGVDPQEGKVYWSDSTLHRISRANLDGSQHEDIITTGLQTTDGLAVDA | |
| ICRKVYWTDTGTNRIEVGNLDGSMRKVLVWQNLDSPRAIVLYHEMGFMYWTDWGENAKLE | |
| RSGMDGSDRAVLINNNLGWPNGLTVDKASSQLLWADAHTERIEAADLNGANRHTLVSPVQ | |
| HPYGLTLLDSYIYWTDWQTRSIHRADKGTCSNVILVRSNLPGLMDMQAVDRAQPLGFNKC | |
| GSRNGGCSHLCLPRPSGFSCACPTGIQLKGDGKTCDPSPETYLLFSSRGSIRRISLDTSD | |
| HTNVHVPVPELNNVISLDYDSVDGKVYYTDVFLDVIRRADLNGSNMETVIGRGLKTTDGL | |
| AVDWVARNLYWTDTGRNTIEASRLDGSCRKVLINNSLDEPRAIAVFPRKGYLFWTDWGHI | |
| AKIERANLDGSERKVLINTDLGWPNGLTLDYDTRRIYWVDAHLDRIESADLNGKLRQVLV | |
| SHVSHPFALTQQDRWIYWTDWQTKSIQRVDKYSGRNKETVLANVEGLMDIIVVSPQRQTG | |
| TNACGVNNGGCTHLCFARASDFVCACPDEPDSQPCSLVPGLVPPAPRATGMSEKSPVLPN | |
| TPPTTLYSSTTRTRTSLEEVEGRCSERDARLGLCARSNDAVPAAPGEGLHISYAIGGLLS | |
| ILLILVVIAALMLYRHKKSKFTDPGMGNLTYSNPSYRTSTQEVKIEAIPKPAMYNQLCYK | |
| KEGGPDHNYTKEKIKIVEGICLLSGDDAEWDDLKQLRSSRGGLLRDHVCMKTDTVSIQAS | |
| SGSLDDTETEQLLQEEQSECSSVHTAATPERRGSLPDTGWKHERKLSSESQV | |

A search of sequence databases reveals that the NOV25 amino acid sequence has 1572 of 1576 amino acid residues (99%) identical to, and 1574 of 1576 amino acid residues (99%) similar to, the 1576 amino acid residue ptnr:SPTREMBL-ACC:O75096 protein from Homo sapiens (Human) (MEGF7). Public amino acid databases include the GenBank databases, SwissProt, PDB and PIR.

NOV25 is expressed in at least adrenal gland/suprarenal gland, bone marrow, brain, bronchus, brown adipose, cartilage, cervix, colon, heart, hypothalamus, lung, peripheral blood, pituitary gland, spinal chord, stomach, testis, thalamus, uterus. This information was derived by determining the tissue sources of the sequences that were included in the invention including but not limited to SeqCalling sources, Public EST sources, Literature sources, and/or RACE sources.

The disclosed NOV25 polypeptide has homology to the amino acid sequences shown in the BLASTP data listed in Table 25C.

TABLE 25C
BLAST results for NOV25

| Gene Index/ | | Length | Identity | Positives | |
|---|---|---|---|---|---|
| Identifier | Protein/Organism | (aa) | (%) | (%) | Expect |
| gi\|17224416\|gb\|AAL36970.1\| | LDLR dan [Mus | 1905 | 1779/1849 | 1809/1849 | 0.0 |
| (AF247637) | musculus] | | (96%) | (97%), | |
| gi\|3449306\|dbj\|BAA32468.1\| | MEGF7 [Homo | 1576 | 1572/1576 | 1574/1576 | 0.0 |
| (AB011540 | sapiens] | | (99%) | (99%) | |
| gi\|6681362\|dbj\|BAA88688.1\| | MEGF7 [Rattus | 1298 | 1248/1298 | 1274/1298 | 0.0 |
| (AB011533) | norvegicus] | | (96%) | (98%) | |
| gi\|17472590\|ref\|XP_ | similar to MEGF7 | 1007 | 992/992 | 992/992 | 0.0 |
| 061753.1\| | (H. sapiens) | | (100%) | (100%) | |
| (XM_061753) | | | | | |
| gi\|14763921\|ref\|XP_ | low density | 859 | 857/859 | 859/859 | 0.0 |
| 035037.1\| | lipoprotein | | (99%) | (99%) | |
| (XM_035037) | receptor-related | | | | |
| | protein 4 [Homo | | | | |
| | sapiens] | | | | |

Tables 25D-E list the domain descriptions from DOMAIN analysis results against NOV25. This indicates that the NOV25 sequence has properties similar to those of other proteins known to contain this domain.

TABLE 25D
Domain Analysis of NOV25

gnl|Smart|smart00192, LDLa, Low-density lipoprotein receptor domain
class A; Cysteine-rich repeat in the low-density lipoprotein (LDL)
receptor that plays a central role in mammalian cholesterol
metabolism. The N-terminal type A repeats in LDL receptor bind the
lipoproteins. Other homologous domains occur in related receptors,
including the very low-density lipoprotein receptor and the LDL
receptor-related protein/alpha 2-macroglobulin receptor, and in
proteins which are functionally unrelated, such as the C9 component of
complement. Mutations in the LDL receptor gene cause familial
hypercholesterolemia.
CD-Length = 38 residues, 97.4% aligned
Score = 64.3 bits (155), Expect = 6e−11

TABLE 25E
Domain Analysis of NOV25

gnl|Smart|smart00135, LY, Low-density lipoprotein-receptor YWTD
domain; Type “B” repeats in low-density lipoprotein (LDL) receptor
that plays a central role in mammalian cholesterol metabolism. Also
present in a variety of molecules similar to gp300/megalin.
CD-Length = 43 residues, 95.3% aligned
Score = 62.0 bits (149), Expect = 3e−10

The domain that characterizes epidermal growth factor (EGF) consists of approximately 50 amino acids, and has been shown to be present, in a more or less conserved form, in a large number of other, mostly animal proteins. EGF-like domains are believed to play a critical role in a number of extracellular events, including cell adhesion and receptor-ligand interactions. Proteins with EGF-like domains often consist of more than 1,000 amino acids, have multiple copies of the EGF-like domain, and contain additional domains known to be involved in specific protein-protein interactions. The list of proteins currently known to contain one or more copies of an EGF-like pattern is large and varied. The functional significance of EGF domains in what appear to be unrelated proteins is not yet clear. However, a common feature is that these repeats are found in the extracellular domain of membrane-bound proteins or in proteins known to be secreted (exception: prostaglandin G/H synthase). The EGF domain includes six cysteine residues which have been shown (in EGF) to be involved in 3 disulfide bonds. The main structure is a two-stranded beta-sheet followed by a loop to a C-terminal short two-stranded sheet. Subdomains between the conserved cysteines vary in length.

To identify proteins containing EGF-like domains, Nakayama et al. (1998) searched a database of long cDNA sequences randomly selected from a human brain cDNA library for those that encode an EGF-like motif. They identified several partial cDNAs encoding novel proteins with EGF-like domains, such as LRP4, which they named MEGF7. The predicted partial LRP4 protein contains 2 EGF-like domains, a calcium binding-type EGF-like domain, HDL receptor-type EGF-like domains, 4 YWTD spacer regions, a transmembrane domain, a cytoplasmic NPXY motif, which is required for clustering and internalization of LDL receptors, and a cytoplasmic tSXV motif, which anchors proteins with a PDZ domain. The sequence and domain organization of LRP4 shows significant similarities to those of members of the LDL receptor family. Northern blot analysis detected rat Megf7 expression in several regions of the brain. Using a radiation hybrid mapping panel, Nakayama et al. (1998) mapped the LRP4 gene to 11p12-p11.2.

The disclosed NOV25 nucleic acid of the invention encoding a MEGF7-like protein includes the nucleic acid whose sequence is provided in Table 25A or a fragment thereof. The invention also includes a mutant or variant nucleic acid any of whose bases may be changed from the corresponding base shown in Table 25A while still encoding a protein that maintains its MEGF7-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV25 protein of the invention includes the MEGF7-like protein whose sequence is provided in Table 25B. The invention also includes a mutant or variant protein any of whose residues may be changed from the corresponding residue shown in Table 25B while still encoding a protein that maintains its MEGF7-like activities and physiological functions, or a functional fragment thereof. In the mutant or variant protein, up to about 1 percent of the residues may be so changed.

The invention further encompasses antibodies and antibody fragments, such as F_{ab} or (F_{ab})₂, that bind immunospecifically to any of the proteins of the invention.

The above defined information for this invention suggests that this MEGF7-like protein (NOV25) may function as a member of a “MEGF7 family”. Therefore, the NOV25 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV25 nucleic acids and proteins of the invention are useful in potential therapeutic applications implicated in diseases including but not limited to various pathologies and disorders as indicated below. For example, a cDNA encoding the MEGF7-like protein (NOV25) may be useful in gene therapy, and the MEGF7-like protein (NOV25) may be useful when administered to a subject in need thereof. By way of nonlimiting example, the compositions of the present invention will have efficacy for treatment of patients suffering from adrenoleukodystrophy, congenital adrenal hyperplasia, hemophilia, hypercoagulation, idiopathic thrombocytopenic purpura, autoimmume disease, allergies, immunodeficiencies, transplantation, graft vesus host; diseases of the brain and nervous system, including Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection; diseases of the respiratory system, including systemic lupus erythematosus, autoimmune disease, asthma, emphysema, scleroderma, allergy, ARDS; diseases and disorders of adipose tissue, reproductive system, colon, circulatory system, spinal chord, digestive system, and endocrine system, or other pathologies or conditions. The NOV25 nucleic acid encoding the MEGF7-like protein of the invention, or fragments thereof, may further be useful in diagnostic applications, wherein the presence or amount of the nucleic acid or the protein are to be assessed.

NOV25 nucleic acids and polypeptides are further useful in the generation of antibodies that bind immuno-specifically to the novel NOV25 substances for use in therapeutic or diagnostic methods. These antibodies may be generated according to methods known in the art, using prediction from hydrophobicity charts, as described in the “Anti-NOVX Antibodies” section below. The disclosed NOV25 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV26

A disclosed NOV26 nucleic acid of 635 nucleotides (also referred to as CG57456-01) encoding a COP-Coated Vesicle Membrane Protein P24 Precursor-like protein is shown in Table 26A. The start and stop codons are in bold letters.

TABLE 26A
NOV26 nucleotide sequence.

(SEQ ID NO:61)

CCCCACTATGGTGACGCTCGCTGAGCTGCTGTTGCTCCAGAACACTCTCCTGACCATGGTCTTGGGCTAT
TTCATCAGCATCCACGCACATGCTGAAGAATGCTTAAGTGAGCATGTCACCTCAGGCACCAAGATGGGCC
TCATCTTCGAAGGTGGCTTCCTGGGCATCAACATGGAGATTACAGGACCTAAGAATAAAAGGATTTATAA
AGGAGACAAAGAATCCAGTGGGAAATACACATTTTCTGCTCACATGGATGGAACAAATACATTTTGTTTT
AGTGACCGAGTGTCCACCATGACTCCAAAGATAGTGATATTCACCATTGATATTGGGGAGGCTACAAAAA
GAGAAGACATGGAAACAGAAGCTCACCAGAACAAACTAGAAGAAATGATCAGTGAGCTGGCTGTGGCCAT
GACAGCTGTACAGCACAAAGAGGAATACACGAAAATCTGGGAGAGGATACACAGAGCCATTAGTGACAAC
ACAAACAGCCCAGTGGTCCTTCGGTGCTTCTTTGAAGCTCTTGTTCTAATTGCCATGACATTGGGACACA
TCTACTACCTGAAGAGATTTTTTGAAGTCCAGAGGGTTGTTTCAAAAGCCTCTTCCTGATGATTCCAAAC
TCATA

In a search of public sequence databases, the NOV26 nucleic acid sequence has 630 of 635 bases (99%) identical to a gb:GENBANK-ID:AF152363|acc:AF152363.1 mRNA from Homo sapiens (Homo sapiens constitutive fragile region FRA3B sequence). Public nucleotide databases include all GenBank databases and the GeneSeq patent database.

The disclosed NOV26 polypeptide (SEQ ID NO:62) encoded by SEQ ID NO:61 has 203 amino acid residues and is presented in Table 26B using the one-letter amino acid code. Signal P, Psort and/or Hydropathy results predict that NOV26 has a signal peptide and is likely to be localized at the plasma membrane with a certainty of 0.4600. The most likely cleavage site for a NOV26 peptide is between amino acids 29 and 30.

TABLE 26B
Encoded NOV26 protein sequence.

| | |
|---|---|
| MVTLAELLLLQNTLLTMVLGYFISIHAHAEECLSEHVTSGTKMGLIFEGGFLGINMEITG | (SEQ ID NO:62) |
| PKNKRIYKGDKESSGKYTFSAHMDGTNTFCFSDRVSTMTPKIVIFTIDIGEATKREDMET | |
| EAHQNKLEEMISELAVANTAVQHKEEYTKIWERIHRAISDNTNSPVVLRCFFEALVLIAM | |
| TLGHIYYLKRFFEVQRVVSKASS | |

A search of sequence databases reveals that the NOV26 amino acid sequence has 156 of 201 amino acid residues (77%) identical to, and 175 of 201 amino acid residues (87%) similar to, the 201 amino acid residue ptnr:SWISSNEW-ACC:Q15363 protein from Homo sapiens (Human) (COP-COATED VESICLE MEMBRANE PROTEIN P24 PRECURSOR (P24A) (RNP24)). Public amino acid databases include the GenBank databases, SwissProt, PDB and PIR.

NOV26 is expressed in at least Eye, placenta, colon, and ovary. This information was derived by determining the tissue sources of the sequences that were included in the invention including but not limited to SeqCalling sources, Public EST sources, Literature sources, and/or RACE sources.

The disclosed NOV26 polypeptide has homology to the amino acid sequences shown in the BLASTP data listed in Table 26C.

TABLE 26C
BLAST results for NOV26

| Gene Index/ | | Length | Identity | Positives | |
|---|---|---|---|---|---|
| Identifier | Protein/Organism | (aa) | (%) | (%) | Expect |
| gi\|17440558\|ref\|XP_067504.1\| | similar to coated vesicle | 271 | 186/192 | 186/192 | e−100 |
| (XM_067504) | membrane protein | | (96%) | (96%) | |
| | [Homo | | | | |
| | sapiens] | | | | |
| gi\|9790015\|ref\|NP_062744.1\| | coated vesicle membrane | 201 | 155/201 | 173/201 | 4−74 |
| (NM_019770) | protein; Sid394p [Mus | | (77%) | (85%), | |
| | musculus] | | | | |
| gi\|1352660\|sp\|P49020\|P24_ | Cop-coated | 196 | 148/196 | 169/196 | 6−74 |
| CRIGR | vesicle membrane | | (75%) | (85%), | |
| | protein p24 precursor | | | | |
| gi\|5803149\|ref\|NP_006806.1\| | coated vesicle membrane | 201 | 156/201 | 175/201 | e−73 |
| (NM_006815) | protein [Homo sapiens] | | (77%) | (86%), | |
| gi\|13929014\|ref\|NP_ | coated vesicle | 201 | 155/201 | 174/201 | e−73 |
| 113910.1\| | membrane protein | | (77%) | (86%), | |
| (NM_031722) | [Rattus | | | | |
| | norvegicus] | | | | |

Table 26D lists the domain descriptions from DOMAIN analysis results against NOV26. This indicates that the NOV26 sequence has properties similar to those of other proteins known to contain this domain.

TABLE 26D
Domain Analysis of NOV26

gnl|Pfam|pfam01105, EMP24_GP25L, emp24/gp25L/p24 family. Mem-
bers of this family are implicated in bringing cargo forward from
the ER and binding to coat proteins by their cytoplasmic domains.
CD-Length = 202 residues, 92.6% aligned
Score = 135 bits (341), Expect = 2e−33

Members of the p24 family of putative cargo receptors are proposed to contain retrograde and anterograde trafficking signals in their cytoplasmic domain to facilitate coat protein binding and cycling in the secretory pathway. The localization and transit of the wild-type chimera from the endoplasmic reticulum (ER) through the Golgi complex involved a glutamic acid residue and a conserved glutamine in the TMD. The TMD glutamic acid mediated the localization of the chimeras to the ER in the absence of the conserved glutamine. Efficient ER exit required the TMD glutamine and was further facilitated by a pair of phenylalanine residues in the cytoplasmic tail. TMD residues of p24 proteins may mediate the interaction with integral membrane proteins of the vesicle budding machinery to ensure p24 packaging into transport vesicles.

Blum et al. (1996) identified a 21-kD rat pancreatic microsomal membrane protein that they designated Tmp21. By probing a human brain cDNA library with a fragment of the rat sequence, they isolated a cDNA encoding human TMP21. The deduced 219-amino acid type I intracellular transmembrane protein contains a signal sequence and is predicted to be located in the lumen of the endoplasmic reticulum. Northern blot analysis detected a 1.4-kb TMP21 transcript. Immunoblot analysis showed that the rat Tmp21 protein is expressed predominantly in the microsomal fraction of pancreatic acinar cells. Horer et al. (1999) determined that a putative TMP21 isoform, TMP21-II, is a neutral pseudogene.

The disclosed NOV26 nucleic acid of the invention encoding a COP-Coated Vesicle Membrane Protein P24 Precursor-like protein includes the nucleic acid whose sequence is provided in Table 26A or a fragment thereof. The invention also includes a mutant or variant nucleic acid any of whose bases may be changed from the corresponding base shown in Table 26A while still encoding a protein that maintains its COP-Coated Vesicle Membrane Protein P24 Precursor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV26 protein of the invention includes the COP-Coated Vesicle Membrane Protein P24 Precursor-like protein whose sequence is provided in Table 26B. The invention also includes a mutant or variant protein any of whose residues may be changed from the corresponding residue shown in Table 26B while still encoding a protein that maintains its COP-Coated Vesicle Membrane Protein P24 Precursor-like activities and physiological functions, or a functional fragment thereof. In the mutant or variant protein, up to about 23 percent of the residues may be so changed.

The invention further encompasses antibodies and antibody fragments, such as F_{ab} or (F_{ab})₂, that bind immunospecifically to any of the proteins of the invention.

The above defined information for this invention suggests that this COP-Coated Vesicle Membrane Protein P24 Precursor-like protein (NOV26) may function as a member of a “COP-Coated Vesicle Membrane Protein P24 Precursor family”. Therefore, the NOV26 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV26 nucleic acids and proteins of the invention are useful in potential therapeutic applications implicated in diseases including but not limited to various pathologies and disorders as indicated below. For example, a cDNA encoding the COP-Coated Vesicle Membrane Protein P24 Precursor-like protein (NOV26) may be useful in gene therapy, and the COP-Coated Vesicle Membrane Protein P24 Precursor-like protein (NOV26) may be useful when administered to a subject in need thereof. By way of nonlimiting example, the compositions of the present invention will have efficacy for treatment of patients suffering from Endometriosis, Fertility, Von Hippel-Lindau (VHL) syndrome, Diabetes, Tuberous sclerosis, or other pathologies or conditions. The NOV26 nucleic acid encoding the COP-Coated Vesicle Membrane Protein P24 Precursor-like protein of the invention, or fragments thereof, may further be useful in diagnostic applications, wherein the presence or amount of the nucleic acid or the protein are to be assessed.

NOV26 nucleic acids and polypeptides are further useful in the generation of antibodies that bind immuno-specifically to the novel NOV26 substances for use in therapeutic or diagnostic methods. These antibodies may be generated according to methods known in the art, using prediction from hydrophobicity charts, as described in the “Anti-NOVX Antibodies” section below. The disclosed NOV16 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV27

A disclosed NOV27 nucleic acid of 1120 nucleotides (also referred to as CG57658-01) encoding a connexin-like protein is shown in Table 27A. The start and stop codons are in bold letters.

TABLE 27A
NOV27 nucleotide sequence.

(SEQ ID NO:63)

GAGGCCATGCCCGCTTCCTCTCTTCCAGGAAAGCTCTGGTTCGTCCTCACGATGCTGCTGCGGATGCTGG
TGATTGTCTTGGCGGGGCGACCCGTCTACCAGGACGAGCAGGAGAGGTTTGTCTGCAACACGCTGCAGCC
GGGATGCGCCAATGTTTGCTACGACGTCTTCTCCCCCGTGTCTCACCTGCGGTTCTGGCTGATCCAGGGC
GTGTGCGTCCTCCTCCCCTCCGCCGTCTTCAGCGTCTATGTCCTGCACCGAGGAGCCACGCTCGCCGCGC
TGGGCCCCCGCCGCTGCCCCGACCCCCGGGAGCCGGCCTCCGGGCAGAGACGCTGCCCGCGGCCATTCGG
GGAGCGCGGCGGCCTCCAGGTGCCCGACTTTTCGGCCGGCTACATCATCCACCTCCTCCTCCGGACCCTG
CTGGAGGCAGCCTTCGGGGCCTTGCACTACTTTCTCTTTGGATTCCTGGCCCCGAAGAAGTTCCCTTGCA
CGCGCCCTCCGTGCACGGGCGTGGTGGACTGCTACGTGTCGCGGCCCACAGAGAAGTCCCTGCTGATGCT
GTTCCTCTGGGCGGTCAGCGCGCTGTCTTTTCTGCTGGGCCTCGCCGACCTGGTCTGCAGCCTGCGGCGG
CGGATGCGCAGGAGGCCGGGACCCCCCACAAGCCCCTCCATCCGGAAGCAGAGCGGAGCCTCAGGCCACG
CGGAGGGACGCCGGACTGACGAGGAGGGTGGGCGGGAGGAAGAGGGGGCACCGGCGCCCCCGGGTGCACG
CGCCGGAGGGGAGGGGGCTGGCAGCCCCAGGCGTACATCCAGGGTGTCAGGGCACACGAAGATTCCGGAT
GAGGATGAGAGTGAGGTGACATCCTCCGCCAGCGAAAAGCTGGGCAGACAGCCCCGGGGCAGGCCCCACC
GAGAGGCCGCCCAGGACCCCAGGGGCTCAGGATCCGAGGAGCAGCCCTCAGCAGCCCCCAGCCGCCTGGC
CGCGCCCCCTTCCTGCAGCAGCCTGCAGCCCCCTGACCCGCCTGCCAGCTCCAGTGGTGCTCCCCACCTG
AGAGCCAGGAAGTCTGAGTGGGTGTGAAAAAAACAGCACCTGGCGGTGCCCCGGGGCTCACGCCTGTAAT

In a search of public sequence databases, the NOV27 nucleic acid sequence, located on chromsome 10 has 1037 of 1097 bases (94%) identical to a gb:GENBANK-ID:AB046017|acc:AB046017.1 mRNA from Macaca fascicularis (Macaca fascicularis brain cDNA, clone:QccE-15512). Public nucleotide databases include all GenBank databases and the GeneSeq patent database.

The disclosed NOV27 polypeptide (SEQ ID NO:64) encoded by SEQ ID NO:63 has 356 amino acid residues and is presented in Table 27B using the one-letter amino acid code. Signal P, Psort and/or Hydropathy results predict that NOV27 has a signal peptide and is likely to be localized in the plasma membrane with a certainty of 0.6400. The most likely cleavage site for a NOV27 peptide is between amino acids 26 and 27.

TABLE 27B
Encoded NOV27 protein sequence.

| | |
|---|---|
| MPASSLPGKLWFVLTMLLRMLVIVLAGRPVYQDEQERFVCNTLQPGCANVCYDVFSPVSH | (SEQ ID NO:64) |
| LRFWLIQGVCVLLPSAVFSVYVLHRGATLAALGPRRCPDPREPASGQRRCPRPFGERGGL | |
| QVPDFSAGYIIHLLLRTLLEAAFGALHYFLFGFLAPKKFPCTRPPCTGVVDCYVSRPTEK | |
| SLLMLFLWAVSALSFLLGLADLVCSLRRRMRRRPGPPTSPSIRKQSCASGHAEGRRTDEE | |
| GGREEEGAPAPPGARAGGEGAGSPRRTSRVSGHTKIPDEDESEVTSSASEKLGRQPRGRP | |
| HREAAQDPRGSGSEEQPSAAPSRLAAPPSCSSLQPPDPPASSSGAPHLRARKSEWV | |

A search of sequence databases reveals that the NOV27 amino acid sequence has 348/348 (100%) identical to TREMBLNEW-ACC:CAC10186 BA425A6.2 (SIMILAR TO CONNEXIN)—Homo sapiens. Public amino acid databases include the GenBank databases, SwissProt, PDB and PIR.

NOV27 is expressed in at least Brain, Lung, Ovary, colon. This information was derived by determining the tissue sources of the sequences that were included in the invention including but not limited to SeqCalling sources, Public EST sources, Literature sources, and/or RACE sources.

The disclosed NOV27 polypeptide has homology to the amino acid sequences shown in the BLASTP data listed in Table 27C.

TABLE 27C
BLAST results for NOV27

| Gene Index/ | | Length | Identity | Positives | |
|---|---|---|---|---|---|
| Identifier | Protein/Organism | (aa) | (%) | (%) | Expect |
| gi\|17489790\|ref\|XP_ | similar to bA425A6.2 | 370 | 349/352 | 351/352 | e−167 |
| 058368.1\| | (similar to connexin) | | (99%) | (99%) | |
| (XM_058368) | [Homo | | | | |
| | sapiens] | | | | |
| gi\|10334641\|emb\|CAC10186.1\| | bA425A6.2 (similar to | 348 | 348/348 | 348/348 | e−163 |
| (AL121749) | connexin) [Homo sapiens] | | (100%) | (100%) | |
| gi\|9280090\|dbj\|BAB01599.1\| | unnamed protein product | 341 | 316/341 | 323/341 | e−159 |
| (AB046017) | [Macaca fascicularis] | | (92%) | (94%) | |
| gi\|17489782\|ref\|XP_ | (similar to connexin) | 341 | 341/341 | 341/341 | e−158 |
| 061277.1\| | [Homo sapiens] | | (100%) | (100%) | |
| (XM_061277) | | | | | |
| gi\|15990849\|emb\|CAC93844.1\| | connexin39 [Mus | 364 | 175/372 | 215/372 | 6e−67 |
| (AJ414562) | musculus] | | (47%) | (57%), | |

Tables 27D-E list the domain descriptions from DOMAIN analysis results against NOV27. This indicates that the NOV27 sequence has properties similar to those of other proteins known to contain this domain.

TABLE 27D
Domain Analysis ot NOV27

gnl|Pfam|pfam00029, connexin, Connexin.
CD-Length = 218 residues, 91.7% aligned
Score = 172 bits (436), Expect = 3e−44

TABLE 27E
Domain Analysis of NOV27

gnl|Smart|smart00037, CNX; Connexin homologues; Connexin channels
participate in the regulation of signaling between developing and
differentiated cell types.
CD-Length = 34 residues, 97.1% aligned
Score = 64.7 bits (156), Expect = 9e−12

Gap junctions were first characterized by electron microscopy as regionally specialized structures on plasma membranes of contacting adherent cells. These structures were shown to consist of cell-to-cell channels. Proteins, called connexins, purified from fractions of enriched gap junctions from different tissues differ. The connexins are designated by their molecular mass. Another system of nomenclature divides gap junction proteins into 2 categories, alpha and beta, according to sequence similarities at the nucleotide and amino acid levels. For example, CX43 is designated alpha-1 gap junction protein, whereas CX32 and CX26 are called beta-1 and beta-2 gap junction proteins, respectively. This nomenclature emphasizes that CX32 and CX26 are more homologous to each other than either of them is to CX43. The connexins are a family of integral membrane proteins that oligomerise to form intercellular channels that are clustered at gap junctions. These channels are specialised sites of cell-cell contact that allow the passage of ions, intracellular metabolites and messenger molecules (with molecular weight<1-2 kD) from the cytoplasm of one cell to its apposing neighbours. They are found in almost all vertebrate cell types, and somewhat similar proteins have been cloned from plant species. Invertebrates utilise a different family of molecules, innexins, that share a similar predicted secondary structure to the vertebrate connexins, but have no sequence identity to them. Vertebrate gap junction channels are thought to participate in diverse biological functions. For instance, in the heart they permit the rapid cell-cell transfer of action potentials, ensuring coordinated contraction of the cardiomyocytes. They are also responsible for neurotransmission at specialised ‘electrical’ synapses. In non-excitable tissues, such as the liver, they may allow metabolic cooperation between cells. In the brain, glial cells are extensively-coupled by gap junctions; this allows waves of intracellular Ca2+ to propagate through nervous tissue, and may contribute to their ability to spatially-buffer local changes in extracellular K+ concentration. The connexin protein family is encoded by at least 13 genes in rodents, with many homologues cloned from other species. They show overlapping tissue expression patterns, most tissues expressing more than one connexin type. Their conductances, permeability to different molecules, phosphorylation and voltage-dependence of their gating, have been found to vary. Possible communication diversity is increased further by the fact that gap junctions may be formed by the association of different connexin isoforms from apposing cells. However, in vitro studies have shown that not all possible combinations of connexins produce active channels. Hydropathy analysis predicts that all cloned connexins share a common transmembrane (TM) topology. Each connexin is thought to contain 4 TM domains, with two extracellular and three cytoplasmic regions. This model has been validated for several of the family members by in vitro biochemical analysis. Both N- and C-termini are thought to face the cytoplasm, and the third TM domain has an amphipathic character, suggesting that it contributes to the lining of the formed-channel. Amino acid sequence identity between the isoforms is 50-80%, with the TM domains being well conserved. Both extracellular loops contain characteristically conserved cysteine residues, which likely form intramolecular disulphide bonds. By contrast, the single putative intracellular loop (between TM domains 2 and 3) and the cytoplasmic C-terminus are highly variable among the family members. Six connexins are thought to associate to form a hemi-channel, or connexon. Two connexons then interact (likely via the extracellular loops of their connexins) to form the complete gap junction channel.

The disclosed NOV27 nucleic acid of the invention encoding a connexin-like protein includes the nucleic acid whose sequence is provided in Table 27A or a fragment thereof. The invention also includes a mutant or variant nucleic acid any of whose bases may be changed from the corresponding base shown in Table 27A while still encoding a protein that maintains its connexin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 6 percent of the bases may be so changed.

The disclosed NOV27 protein of the invention includes the connexin-like protein whose sequence is provided in Table 27B. The invention also includes a mutant or variant protein any of whose residues may be changed from the corresponding residue shown in Table B while still encoding a protein that maintains its connexin-like activities and physiological functions, or a functional fragment thereof. In the mutant or variant protein, up to about 0 percent of the residues may be so changed.

The invention further encompasses antibodies and antibody fragments, such as F_{ab} or (F_{ab})₂, that bind immunospecifically to any of the proteins of the invention.

The above defined information for this invention suggests that this connexin-like protein (NOV27) may function as a member of a “connexin family”. Therefore, the NOV27 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV27 nucleic acids and proteins of the invention are useful in potential therapeutic applications implicated in diseases including but not limited to various pathologies and disorders as indicated below. For example, a cDNA encoding the connexin-like protein (NOV27) may be useful in gene therapy, and the connexin-like protein (NOV27) may be useful when administered to a subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Obesity, Endometriosis, Fertility, Hemophilia, Hypercoagulation, Idiopathic thrombocytopenic purpura, Immunodeficiencies, Graft vesus host, Autoimmune disease, Renal artery stenosis, Interstitial nephritis, Glomerulonephiritis, Polycystic kidney disease, Systemic lupus erythematosus, Renal tubular acidosis, IgA nephropathy, Hypercalceimia, Lesch-Nyhan syndrome, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, or other pathologies or conditions. The NOV27 nucleic acid encoding the connexin-like protein of the invention, or fragments thereof, may further be useful in diagnostic applications, wherein the presence or amount of the nucleic acid or the protein are to be assessed.

NOV27 nucleic acids and polypeptides are further useful in the generation of antibodies that bind immuno-specifically to the novel NOV27 substances for use in therapeutic or diagnostic methods. These antibodies may be generated according to methods known in the art, using prediction from hydrophobicity charts, as described in the “Anti-NOVX Antibodies” section below. The disclosed NOV27 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV28

A disclosed NOV28 nucleic acid of 1234 nucleotides (also referred to as CG57662-01) encoding a-like protein is shown in Table 28A. The start and stop codons are in bold letters.

TABLE 28A
NOV28 nucleotide sequence.

(SEQ ID NO:65)

TAATAATCTTTTTTTAAAACTCCCTAACAGGATGTGTGGCAGGTTCCTGAGGTGGTGGCTGCTGGCGGAG
GAGAGCTGGCACTCCACCCCCGTGGGGCGCCTCCTGTTTCCCGTGCTCCTGGGATTCCGCCTTGTGCTGC
TGGCTGCCAGTGGGCCTGGAGTCTATGGCGATGAGCAGAGTGAATTCGTGTGTCACACCCAGCAGCCGGG
CTGCAAGGCTGCCTGCTTCGATGCCTTCCACCCGCTCTCCCCGCTGCGTTTCTGGGTCTTCCAGGTCATC
TTGGTGGCTGTACCTAGCGTCCTCTACATGGGTTTCACTCTGTATCACGTGATCTGGCACTGGGAAGAAT
CAAGAAAGGGGACGGAGGAAGAGGACACCCTGATCCAGGGAGGGGAGAGCAGCAGAGATACCCCAGGGGC
TGGAAGCCTCAGGCTGCTCCGAGCTTATGTGGCTCAGCTGGGAGCTCAGCTGGTCCTGGAGGGGACAGCG
CCGGGGTTGCAGTACCACCTGTATGGGTTCCAGATGCCCAGCTCCTTTGCATGTGGCCAAGAGCCTTGCC
CGTATAGATTAACTTGCACCTTTTCCCACCCCTCGGAGAAGATCATCTTTCTAAAAGCCATGTTTGGGGT
CAGTGGGTTCCGTCTCTTGTTCACTCTTTTGGAGATTGTGCTTCTGGGTCTGGGAAGACTGTGTAAGCCC
CTGCGGAACTTCCTGGGTGGGGCCTCTTCCTCCACCCACGCCCTGGCCCTGAGCAGCAAAAGGAACCTCC
AGCAGACACTGGGAGCCATCCATCGGCCTGGTCAGCCTTGTTCCATTTCAGAGACCATGTTCCCCACAGC
CCCAGTGACTAGGGGTGACATCTCCCGACCTCCCCCACCTGTGGATATGGCCAAGTCGAGGTACCGGTTA
ACCAAAGATGCTGAAGCAGTGAAGAACCAGCCATCCCCTAATACGCAGGATGGTTATATTGATTATGTCA
AACTGAAAACTTTGGAGAAACTCCTCTCTCAGAAAGCGATAACTGGGCCAGACACGGTGGCTCATGCCTG
TAATCCCAGCATTTTGGGAGGCCTAGGCAGGTGGATCACTGGAGGTCAGGAGTTCAAGACCAGCCAGGCC
AACATGGTGAAACCCGTGTCTACTAAAACTACAAAAATTCTGGGCATGGTGGTGGGCGTCTGTAATCCCA
GCTACTTGAGAGGCTGAGGCAGGAGAATTGCTTGAACCTGGGAG

In a search of public sequence databases, the NOV28 nucleic acid sequence, located on chromsome 7 has 206 of 244 bases (84%) identical to a gb:GENBANK-ID:AP000692|acc:AP000692.1 mRNA from Homo sapiens (Homo sapiens genomic DNA, chromosome 21q22.2, PAC clone:24J14, CBR1-HLCS region). Public nucleotide databases include all GenBank databases and the GeneSeq patent database.

The disclosed NOV28 polypeptide (SEQ ID NO:66) encoded by SEQ ID NO:65 has 391 amino acid residues and is presented in Table 28B using the one-letter amino acid code. Signal P, Psort and/or Hydropathy results predict that NOV28 has a signal peptide and is likely to be localized in the plasma membrane with a certainty of 0.6000. The most likely cleavage site for a NOV28 peptide is between amino acids 46 and 47.

TABLE 28B
Encoded NOV28 protein sequence.

| | |
|---|---|
| MCGRFLRWWLLAEESWHSTPVGRLLFPVLLGFRLVLLAASGPGVYGDEQSEFVCHTQQPG | (SEQ ID NO:66) |
| CKAACFDAFHPLSPLRFWVFQVILVAVPSVLYMGFTLYHVIWHWEESRKGTEEEDTLLQG | |
| GESSRDTPGAGSLRLLRAYVAQLGAQLVLEGTAPGLQYHLYGFQMPSSFACGQEPCPYRL | |
| TCTFSHPSEKIIFLKAMFGVSGFRLLFTLLEIVLLGLGRLCKPLRNFLGGASSSSHALAL | |
| SSKRNLQQTLGAIHRPGQPCSISETMFPTAPVTRGDISRPPPPVDMAKSRYRLTKDAEGV | |
| KNQPSPNTQDGYIDYVKLKTLEKLLSQKAITGPDTVAHACNPSILGGLGRWITGGQEFKT | |
| SQANMVKPVSTKTTKILGMVVGVCNPSYLRG | |

A search of sequence databases reveals that the NOV28 amino acid sequence has 108/108 (100%) amino acids identical with ptnr:SPTREMBL-ACC:O60387 WUGSC:H_DJ0604G05.3 PROTEIN—Homo sapiens (Human). Public amino acid databases include the GenBank databases, SwissProt, PDB and PIR.

NOV28 is expressed in at least Brain, Breast, Colon, Gall bladder, Germ Cell, Heart, Kidney, Liver, Ovary, Pancreas, Prostate, Stomach, Testis, Whole embryo, brain, breast, breast_nornal, colon, colon_ins, head_neck, lung, nervous_tumor, prostate, prostate_normal, prostate_tumor, stomach. This information was derived by determining the tissue sources of the sequences that were included in the invention including but not limited to SeqCalling sources, Public EST sources, Literature sources, and/or RACE sources.

The disclosed NOV28 polypeptide has homology to the amino acid sequences shown in the BLASTP data listed in Table 28C.

TABLE 28C
BLAST results for NOV28

| Gene Index/ | | Length | Identity | Positives | |
|---|---|---|---|---|---|
| Identifier | Protein/Organism | (aa) | (%) | (%) | Expect |
| gi\|3006230\|gb\|AAC09485.1\| | gap junction | 207 | 207/250 | 207/250 | e−103 |
| (AC004522) | protein; similar | | (82%) | (82%) | |
| | to P36383 | | | | |
| | (PID: g544117) | | | | |
| | [Homo sapiens] | | | | |
| gi\|17978264\|ref\|NP_ | gap junction | 258 | 135/219 | 167/219 | 2e−75 |
| 536698.1\| | membrane channel | | (61%) | (75%) | |
| (NM_080450) | protein epsilon | | | | |
| | 1; connexin 29 | | | | |
| | [Mus musculus] | | | | |
| gi\|18566654\|ref\|XP_ | hypothetical | 222 | 100/115 | 103/115 | 3e−51 |
| 095131.1\| | protein XP_095131 | | (86%) | (88%) | |
| (XM_095131) | [Homo sapiens] | | | | |
| gi\|6680007\|ref\|NP_032148.1\| | gap junction | 396 | 96/250 | 136/250 | 5e−41 |
| (NM_008122) | membrane channel | | (38%) | (54%) | |
| | protein alpha 7; | | | | |
| | connexin 45 [Mus | | | | |
| | musculus] | | | | |
| gi\|4885169\|ref\|NP_005488.1\| | gap junction | 396 | 96/250 | 136/250 | 8e−41 |
| (NM_005497) | protein, alpha 7, | | (38%) | (54%) | |
| | 45 kD (connexin | | | | |
| | 45) [Homo | | | | |
| | sapiens] | | | | |

Table 28D lists the domain descriptions from DOMAIN analysis results against NOV28. This indicates that the NOV28 sequence has properties similar to those of other proteins known to contain this domain.

TABLE 28D
Domain Analysis of NOV28

gnl|Pfam|pfam00029, connexin, Connexin.

CD-Length = 218 residues, 90.4% aligned

Score = 173 bits (438), Expect = 2e−44

The disclosed NOV28 nucleic acid of the invention encoding a connexin-like protein includes the nucleic acid whose sequence is provided in Table 28A or a fragment thereof. The invention also includes a mutant or variant nucleic acid any of whose bases may be changed from the corresponding base shown in Table 28A while still encoding a protein that maintains its connexin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 16 percent of the bases may be so changed.

The disclosed NOV28 protein of the invention includes the connexin-like protein whose sequence is provided in Table 28B. The invention also includes a mutant or variant protein any of whose residues may be changed from the corresponding residue shown in Table 28B while still encoding a protein that maintains its connexin-like activities and physiological functions, or a functional fragment thereof. In the mutant or variant protein, up to about 0 percent of the residues may be so changed.

The invention further encompasses antibodies and antibody fragments, such as F_{ab} or (F_{ab})₂, that bind immunospecifically to any of the proteins of the invention.

The above defined information for this invention suggests that this connexin-like protein (NOV28) may function as a member of a “connexin family”. Therefore, the NOV28 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV28 nucleic acids and proteins of the invention are useful in potential therapeutic applications implicated in diseases including but not limited to various pathologies and disorders as indicated below. For example, a cDNA encoding the connexin-like protein (NOV28) may be useful in gene therapy, and the connexin-like protein (NOV28) may be useful when administered to a subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Obesity, Endometriosis, Fertility, Hemophilia, Hypercoagulation, Idiopathic thrombocytopenic purpura, Immunodeficiencies, Graft vesus host, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, or other pathologies or conditions. The NOV28 nucleic acid encoding the connexin-like protein of the invention, or fragments thereof, may further be useful in diagnostic applications, wherein the presence or amount of the nucleic acid or the protein are to be assessed.

NOV28 nucleic acids and polypeptides are further useful in the generation of antibodies that bind immuno-specifically to the novel NOV28 substances for use in therapeutic or diagnostic methods. These antibodies may be generated according to methods known in the art, using prediction from hydrophobicity charts, as described in the “Anti-NOVX Antibodies” section below. The disclosed NOV28 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV29

A disclosed NOV29 nucleic acid of 1400 nucleotides (also referred to as CG57664-01) encoding a MHC Class I antigen-like protein is shown in Table 29A.

TABLE 29A
NOV29 nucleotide sequence.

(SEQ ID NO:67)

ATTCTCCCCAAACGCCAGGGATGGGGGTCATGGCTCCCCGAACCCTCCTCCTGCTGCTCTTGGGGGCCCT
GGCCCTGACCGAGACCTGGGCCGGTGAGTGCGGGGTCGGGAGGGAAAGGGCCTCTGCGGGGAGAAGCGAG
TGGCCCGCCCGGCCCGGGGAGCCGCGCCTCAGCCTCTCCTCGCCTCCAGGCTCCCACTCCTTGAGGTATT
TCAGCACCGCAGTGTCCCACCCCGGCCGCGGGGAGCCCCGGTTCATCGCCGTGGGCTACGTGGACGACAC
AGAGTTCGTGCGGTTCGACAGCGACTCCGTGAGTCCGAGGATGGAGCGGCGGGCGCCGTGGGTGGAGCAG
GAGGGGCTGGAGTATTGGGACCAGGAGACACGGAACGCCAAGGGCCACGCGCAGATTTACCGAGTGAACC
TGCGGACCCTGCTCCGCTATTACAACCAGAGCGAGGCCGGTGGTTCTCACACCATCCAGAGGAAGCATGA
CTGCGACGTGGGCCCGACAGGCGGGCCCGACAGGCGCCTCCTCCGCAGGTATGAACAGTTCGCCTACGAT
GGCAAGGATTACATCGCCCTGAACGAGGACCTGCCCTCCTGGACCCCCGCCAACACAGCGGCTCAGATCT
CCCAGCACAAGTGGGAAGCGGACAAATACTCAGAGCAGGTCAGGGCCTACCTGAGGGCAAGTGCATGGAG
TGGCGAGGGCAAGTGCATGGAGTGGCTCCGCAGACACCTGGAGAACGGGAAGGAGACGCTGCAGCGCGCG
TCAGATCCCCCAAAGGCACATGTGACCCAGCACCCCGTCTCTGACCATGAGGCCACCCTTGAGGTGCTGG
GCCCTGGGCCTCTACCCTTCAGGTGCTGGGCCCTGGGCCTCTACCCTGCGGAGATCACACTGACCTGGCA
GCAGGATGGGGAGGACCAGACCCAGGACACGGAGCTTGTGGAGACCAGGCCTGCAGGGGACGGAACCTTC
CAGAAGTGGGTGGCTGTAGTGGTGCCTTCCGGAGAGGAGCAGAGATACATGTGCCATGTGCAGCATGAGG
GGCTGCCAGAGCCCCTCACCCTGAGATGGCCCTCACCTCCCTCTCCTTTCCCAGAGCCGTCTTCTCAGCC
CACCATCCCCATCGTGGGCATCGTTGCTGGCCTGTTTCTCCTTGGAGCTGTGGTCACTGGAGCTGTGGTT
GCTGCTGTGATGAAGAGGAAGAAAAGCTCAGGTAGGGAAGGGGTGAGAGGTGGGATCTGGGTTTTCTTGT
TCCACTGTGGGTTTCAAGCCACAGGTAGAATTGTGACTTGCTTCATCACTGGGAAGCACCGTCCACACAC
AGGCCGACCTAGCCTGGGGCCCTGTGTGCCAACACTTGCTCTTTTGTGAAGCACATGTGAAAACGAAGGA

In a search of public sequence databases, the NOV29 nucleic acid sequence, located on chromsome 6 has 332 of 353 bases (94%) identical to a gb:GENBANK-ID:HUMHLA92|acc:M96338.1 mRNA from Homo sapiens (Homo sapiens HLA-92 gene sequence). Public nucleotide databases include all GenBank databases and the GeneSeq patent database.

The disclosed NOV29 polypeptide (SEQ ID NO:68) encoded by SEQ ID NO:67 has 452 amino acid residues and is presented in Table 29B using the one-letter amino acid code. Signal P, Psort and/or Hydropathy results predict that NOV29 has a signal peptide and is likely to be localized at the plasma membrane with a certainty of 0.4600. The most likely cleavage site for a NOV29 peptide is between amino acids 24 and 25.

TABLE 29B
Encoded NOV29 protein sequence.

| | |
|---|---|
| MGVMAPRTLLLLLLGALALTETWAGECGVGRERASAGRSEWPARPGEPRLSLSSPPGSHS | (SEQ ID NO:68) |
| LRYFSTAVSQPGRGEPRFIAVGYVDDTEFVRFDSDSVSPRMERRAPWVEQEGLEYWDQET | |
| RNAKGHAQIYRVNLRTLLRYYNQSEAGGSHTIQRKHDCDVGPTGGPDRRLLRRYEQFAYD | |
| GKDYIALNEDLPSWTAANTAAQISQHKWEADKYSEQVRAYLRASAWSGEGKCMEWLRRHL | |
| ENGKETLQRASDPPKAHVTQHPVSDHEATLEVLGPGPLPLRCWALGLYPAEITLTWQQDG | |
| EDQTQDTELVETRPAGDGTFQKWVAVVVPSGEEQRYMCHVQHEGLPEPLTLRWPSPPSPF | |
| PEPSSQPTIPIVGIVAGLFLLGAVVTGAVVAAVMKRKKSSGREGVRGGIWVFLFHCGFQA | |
| TGRIVTCFITGKHRPHTGRPSLGPCVPTLALL | |

A search of sequence databases reveals that the NOV29 amino acid sequence has 158/223 (70%) identity and 177/223 (79%) similarity with SPTREMBL-ACC:Q9TPL2 MHC CLASS I ANTIGEN—Pan troglodytes (Chimpanzee). Public amino acid databases include the GenBank databases, SwissProt, PDB and PIR.

NOV29 is expressed in at least Bone Marrow, Dermis, Hippocampus, Placenta, and Tonsils. Expression information was derived from the tissue sources of the sequences that were included in the derivation of the sequence of CG57664-01. The sequence is predicted to be expressed in the following tissues because of the expression pattern of (GENBANK-ID: gb:GENBANK-ID:HUMHLA92|acc:M96338.1) a closely related Homo sapiens HLA-92 gene sequence homolog in species Homo sapiens: Lymphoblastoid cell line. This information was derived by determining the tissue sources of the sequences that were included in the invention including but not limited to SeqCalling sources, Public EST sources, Literature sources, and/or RACE sources.

The disclosed NOV29 polypeptide has homology to the amino acid sequences shown in the BLASTP data listed in Table 29C.

TABLE 29C
BLAST results for NOV29

| Gene Index/ | | Length | Identity | Positives | |
|---|---|---|---|---|---|
| Identifier | Protein/Organism | (aa) | (%) | (%) | Expect |
| gi\|8117819\|gb\|AAF72785.1\| | MHC class I | 365 | 275/404 | 300/404 | e−141 |
| AF168404_1 | antigen [Pan troglodytes] | | (68%) | (74%), | |
| (AF168404) | | | | | |
| gi\|8117799\|gb\|AAF72776.1\| | MHC class I | 365 | 276/404 | 301/404 | e−141 |
| AF168395_1 | antigen [Pan troglodytes] | | (68%) | (74%) | |
| (AF168395) | | | | | |
| gi\|2118771\|pir\|\|I54493 | MHC class I | 365 | 271/404 | 299/404 | e−141 |
| | histocompatibility | | (67%) | (73%), | |
| | antigen HLA-A | | | | |
| | alpha chain | | | | |
| | precursor human | | | | |
| gi\|8117808\|gb\|AAF72780.1\| | MHC class I | 365 | 275/404 | 300/404 | e−140 |
| AF168399_1 | antigen [Pan troglodytes] | | (68%) | (74%), | |
| (AF168399) | | | | | |
| gi\|2251146\|emb\|CAA65501.1\| | human leukocyte | 365 | 270/404 | 300/404 | e−140 |
| (X96724) | antigen [Homo | | (66%) | (73%), | |
| | sapiens] | | | | |

Tables 29D-E list the domain descriptions from DOMAIN analysis results against NOV29. This indicates that the NOV29 sequence has properties similar to those of other proteins known to contain this domain.

TABLE 29D
Domain Analysis of NOV29

gnl|Pfam|pfam00129, MHC_I, Class I Histocompatibility antigen,
domains alpha 1 and 2

CD-Length = 179 residues, 96.6% aligned

Score = 248 bits (634), Expect = 4e−67

TABLE 29E
Domain Analysis of NOV29

gnl|Smart|smart00407, IGcl, Immunoglobulin C-Type

CD-Length = 75 residues, 96.0% aligned

Score = 59.7 bits (143), Expect = 4e−10

The major histocompatibility complex (MHC) encodes the class I and class II families of glycoproteins that present peptides for immunorecognition by cytotoxic and helper T lymphocytes, respectively. Class I molecules bind peptides generated by degradation of proteins intracellularly, whereas class II molecules associate mainly with peptides derived from endocytosed extracellular proteins. Two genes encode components of the proteasome complex, which degrades cytosolic proteins and may generate antigenic peptides. Two closely linked genes, PSF1 and PSF2, encode subunits of a transporter, which presumably translocates peptides into an exocytic compartment where they associate with class I molecules. The location of these genes in the MHC in close linkage to the class I and class II gene families suggests that they coevolved to optimize functional interactions.

The disclosed NOV29 nucleic acid of the invention encoding a MHC Class I antigen-like protein includes the nucleic acid whose sequence is provided in Table 29A or a fragment thereof. The invention also includes a mutant or variant nucleic acid any of whose bases may be changed from the corresponding base shown in Table 29A while still encoding a protein that maintains its MHC Class I antigen-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 6 percent of the bases may be so changed.

The disclosed NOV29 protein of the invention includes the MHC Class I antigen-like protein whose sequence is provided in Table 29B. The invention also includes a mutant or variant protein any of whose residues may be changed from the corresponding residue shown in Table 29B while still encoding a protein that maintains its MHC Class I antigen-like activities and physiological functions, or a functional fragment thereof. In the mutant or variant protein, up to about 30 percent of the residues may be so changed.

The invention further encompasses antibodies and antibody fragments, such as F_{ab} or (F_{ab})₂, that bind immunospecifically to any of the proteins of the invention.

The above defined information for this invention suggests that this MHC Class I antigen-like protein (NOV29) may function as a member of a “MHC Class I antigen family”. Therefore, the NOV29 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV29 nucleic acids and proteins of the invention are useful in potential therapeutic applications implicated in diseases including but not limited to various pathologies and disorders as indicated below. F-or example, a cDNA encoding the MHC Class I antigen-like protein (NOV29) may be useful in gene therapy, and the MHC Class I antigen-like protein (NOV29) may be useful when administered to a subject in need thereof. By way of nonlimiting example, the compositions of the present invention will have efficacy for treatment of patients suffering from Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Tonsilitis, Hemophilia, hypercoagulation, Idiopathic thrombocytopenic purpura, autoimmume disease, allergies, immunodeficiencies, transplantation, Graft versus host, or other pathologies or conditions. The NOV29 nucleic acid encoding the MHC Class I antigen-like protein of the invention, or fragments thereof, may further be useful in diagnostic applications, wherein the presence or amount of the nucleic acid or the protein are to be assessed.

NOV29 nucleic acids and polypeptides are further useful in the generation of antibodies that bind immuno-specifically to the novel NOV29 substances for use in therapeutic or diagnostic methods. These antibodies may be generated according to methods known in the art, using prediction from hydrophobicity charts, as described in the “Anti-NOVX Antibodies” section below. The disclosed NOV29 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV30

A disclosed NOV30 nucleic acid of 1225 nucleotides (also referred to as CG57666-01) encoding a MHC Class I antigen-like protein is shown in Table 30A. The start and stop codons are in bold letters.

TABLE 30A
NOV30 nucleotide sequence.

(SEQ ID NO:69)

ACGCCGAGGATGGGGTCATGGCGTCCCAAACCCTCCTCCTGCTCCTCTTGGGGGCCCTGGCCCTGACCGA
CACCTGGGCGGGTACCCACTCCATAAGGTATTTCAGCACCGCCGTGTCCCGGCCGGGTCGCGGGGAGCCC
CGGGGTACCCACTCCATAAGGTATTTCAGCACCGCCGTGTCCCGGCCGGGTCGCGGGGAGCCCCGGTACA
TCGCAGTGGGCTACGTGGACGACACGCAGTTCGTGCGGTTCGACAGCGACGCGGCGACTCCGAGGATGGA
GCCGCAGGCGCCGTGGTTGGAGCAGGAGGGACCGGAGTATTGGGACCGGAGCACACCGAACATCAGGCCC
GCGCACAGACTGACAAGACTGAACCTGCCCATGCCGCGCCGCTACTACCACCAGAGCGGGTCTAACACCC
TCCAGATAATGTATGGCTGCGACTTGGGGCTGGAAGGGCGCCTCCTCCGCGGGTATGAACAGCACGCCAA
CGATGGCAAAGATTACATCGCCCTAAACGAGGACCTGAGCTCTTGGACCGCGGCGGCCATGGCGGCTCAG
ATTACCCAGCGCAAGTGGGAGGCGGCCCATGAGGCGGAGCAGCAGAGAGCCTACCTGGAGGGCACGTGCG
TGGAGTGGCTCCGCAGATACCTGGAGAACGGGAAGGAGACGCTGCAGCGCACTACCCCCCCCCCCAAGAC
ACATATGATCCACCATTCCGTCTCTGACTATAAGGCCACCCTGAGATGCTGGGCCCTGGGCTTCTACCCT
GTGGAGATCACACTGACCTGGCAGCAGGATGGAGAGGACCAGACTCAGGACATGGAGCTTGTAGAGACCA
GGCCTGCAGGGGATGGAAACTTCCAGAAGTGGGCAGCTGTGGTGGTGCCTTCTGGAGAGGAACAGAGATA
CATGTGCCATGTGCAGCATGAGGGGTTGCCCAACCCCCTCACCCTGAGATGGGAGCAGTCTTCTCAGCCC
ACCATCCCCATCGTGGGTATCGTTGCTGGCCTGGTTCTCCTTGGAGCTGTAGTCACTGGAGCTGTGGTTT
CTGCTGTGATGTGCAGGAAGAACTCATTTTGTTCTACCCCAGGCAGCAACCATGCGCAGGGTTCTGATGT
GTCTCTCACGGCTTGTAAAGGTGAGACGCTGGGGGACCTGATGTGTGGGGGGTGTTGGGGGCAATAGTGG
ATGCAGCTGTGCTATGGGGTTTCTTTGAATTGGAT

In a search of public sequence databases, the NOV30 nucleic acid sequence, located on chromsome 6 has 265 of 271 bases (97%) identical to a gb:GENBANK-ID:AF055066|acc:AF055066.1 mRNA from Homo sapiens (Homo sapiens MHC class I region). Public nucleotide databases include all GenBank databases and the GeneSeq patent database.

The disclosed NOV30 polypeptide (SEQ ID NO:70) encoded by SEQ ID NO:69 has 389 amino acid residues and is presented in Table 30B using the one-letter amino acid code. Signal P, Psort and/or Hydropathy results predict that NOV30 has a signal peptide and is likely to be localized at the plasma membrane with a certainty of 0.4600. The most likely cleavage site for a NOV30 peptide is between amino acids 21 and 22.

TABLE 30B
Encoded NOV30 protein sequence.

| | |
|---|---|
| MASQTLLLLLLGALALTETWAGTHSIRYFSTAVSRPGRGEPRGTHSIRYFSTAVSRPGRG | (SEQ ID NO:70) |
| EPRYIAVGYVDDTQFVRFDSDAATPRMEPQAPWLEQEGPEYWDRSTPNIRPAHRLTRVNL | |
| PMPRRYYHQSGSNTLQIMYGCDLGLEGRLLRGYEQHANDGKDYIALNEDLSSWTAAAMAA | |
| QITQRKWEAAHEAEQQRAYLEGTCVEWLRRYLENGKETLQRTTPPPKTHMIHHSVSDYKA | |
| TLRCWALGFYPVEITLTWQQDGEDQTQDMELVETRPAGDGNFQKWAAVVVPSGEEQRYMC | |
| HVQHEGLPKPLTLRWEQSSQPTIPIVGIVAGLVLLGAVVTGAVVSAVMCRKNSFCSTPGS | |
| NHAQGSDVSLTACKGETLGDLMCGGCWGQ | |

A search of sequence databases reveals that the NOV30 amino acid sequence has 258/338 (76%) identity and 284/338 (84%) similarity with SPTREMBL-ACC:Q31602 MHC CLASS I ANTIGEN—Homo sapiens. Public amino acid databases include the GenBank databases, SwissProt, PDB and PIR.

NOV30 is expressed in at least Bone Marrow, Dermis, Hippocampus, Placenta, Tonsils. This information was derived by determining the tissue sources of the sequences that were included in the invention including but not limited to SeqCalling sources, Public EST sources, Literature sources, and/or RACE sources.

The disclosed NOV30 polypeptide has homology to the amino acid sequences shown in the BLASTP data listed in Table 30C.

TABLE 30C
BLAST results for NOV30

| Gene Index/ | | Length | Identity | Positives | |
|---|---|---|---|---|---|
| Identifier | Protein/Organism | (aa) | (%) | (%) | Expect |
| gi\|8571968\|gb\|AAF76942.1\| | MHC class I | 365 | 283/383 | 313/383 | e−155 |
| AF179640_1 | antigen [Pan troglodytes] | | (73%) | (80%) | |
| (AF179640) | | | | | |
| gi\|8571970\|gb\|AAF76943.1\| | MHC class I | 363 | 282/383 | 312/383 | e−155 |
| AF179641_1 | antigen [Pan troglodytes] | | (73%) | (80%) | |
| (AF179641) | | | | | |
| gi\|8117808\|gb\|AAF72780.1\| | MHC class I | 365 | 282/383 | 312/383 | e−155 |
| AF168399_1 | antigen [Pan troglodytes] | | (73%) | (80%) | |
| (AF168399) | | | | | |
| gi\|2118771\|pir\|\|I54493 | MHC class I | 365 | 280/383 | 312/383 | e−154 |
| | histocompatibility | | (73%) | (80%) | |
| | antigen HLA-A | | | | |
| | alpha chain | | | | |
| | precursor-human | | | | |
| gi\|6049049\|gb\|AAF02442.1\| | MHC class I | 365 | 281/383 | 311/383 | e−153 |
| (AF115463) | antigen [Pan troglodytes] | | (73%) | (80%) | |

Tables 30D-E list the domain descriptions from DOMAIN analysis results against NOV30. This indicates that the NOV30 sequence has properties similar to those of other proteins known to contain this domain.

TABLE 30D
Domain Analysis of NOV30

gnl|Pfam|pfam00129, MHC_I, Class I Histocompatibility antigen,
domains alpha 1 and 2.

CD-Length = 179 residues, 100.0% aligned

Score = 245 bits (626), Expect = 3e−66

TABLE 30E
Domain Analysis of NOV30

gnl|Smart|smart00407, IGc1, Immunoglobulin C-Type

CD-Length = 75 residues, 100.0% aligned

Score = 75.5 bits (184), Expect = 5e−15

The disclosed NOV30 nucleic acid of the invention encoding a MHC Class I antigen-like protein includes the nucleic acid whose sequence is provided in Table 30A or a fragment thereof. The invention also includes a mutant or variant nucleic acid any of whose bases may be changed from the corresponding base shown in Table 30A while still encoding a protein that maintains its MHC Class I antigen-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 3 percent of the bases may be so changed.

The disclosed NOV30 protein of the invention includes the MHC Class I antigen-like protein whose sequence is provided in Table 30B. The invention also includes a mutant or variant protein any of whose residues may be changed from the corresponding residue shown in Table 30B while still encoding a protein that maintains its MHC Class I antigen-like activities and physiological functions, or a functional fragment thereof. In the mutant or variant protein, up to about 24 percent of the residues may be so changed.

The invention further encompasses antibodies and antibody fragments, such as F_{ab} or (F_{ab})₂, that bind immunospecifically to any of the proteins of the invention.

The above defined information for this invention suggests that this MHC Class I antigen-like protein (NOV30) may function as a member of a “MHC Class I antigen family”. Therefore, the NOV30 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV30 nucleic acids and proteins of the invention are useful in potential therapeutic applications implicated in diseases including but not limited to various pathologies and disorders as indicated below. For example, a cDNA encoding the MHC Class I antigen-like protein (NOV30) may be useful in gene therapy, and the MHC Class I antigen-like protein (NOV30) may be useful when administered to a subject in need thereof. By way of nonlimiting example, the compositions of the present invention will have efficacy for treatment of patients suffering from Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Tonsilitis, Hemophilia, hypercoagulation, Idiopathic thrombocytopenic purpura, autoimmume disease, allergies, immunodeficiencies, transplantation, Graft vesus host, or other pathologies or conditions. The NOV30 nucleic acid encoding the MHC Class I antigen-like protein of the invention, or fragments thereof, may further be useful in diagnostic applications, wherein the presence or amount of the nucleic acid or the protein are to be assessed.

NOV30 nucleic acids and polypeptides are further useful in the generation of antibodies that bind immuno-specifically to the novel NOV30 substances for use in therapeutic or diagnostic methods. These antibodies may be generated according to methods known in the art, using prediction from hydrophobicity charts, as described in the “Anti-NOVX Antibodies” section below. The disclosed NOV30 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV31

A disclosed NOV31 nucleic acid of 1159 nucleotides (also referred to as CG57668-01) encoding a MHC Class I antigen-like protein is shown in Table 31A. The start and stop codons are in bold letters.

TABLE 31A
NOV31 nucleotide sequence.

(SEQ ID NO:71)

TCTCCCCAGACGCCGAGGATGGTGCTCATGGCGCCCCGAACCCTCCTCCTGCTGCTCTCAGGGGCCCTGA
CCCAGACCTGGGCGCGTTCCCACTCCATGAGGTATTTCTACACCACCATGTCCCGGCCCGCCCGCCGGGA
GCCCCGCTTCATCTCCGTCGGCTACGTGGACTATACGCAGTTCGTGCGGTTCGACAGCGACGACGCGAGT
CCGAGAGAGGAGCCGCGGGCGCCGTGGATGGAGCGGGAGGGGCCGGAGTATTGGGACCGGAACACACAGA
TCTGCAAGGCCCAAGCACGGACTGAACGAGAGAACCTGCGGATCGCGCTCCGCTACTACAACCAGAGCGA
GGGCGGTGGTTCCCACACCATGCAGGTGATGTATGGCTGCGACGTGGGGCCCGACGGGCGCTTCCTCCGC
GGGTATGAACACCACGCCTACGACGGCAAGGATTACATCGCTCTGAACGAGGACCTGCGCTCCTGGACCG
CGGCGGACATGGCAGCTCAGATCACCAAGCGCAAGTGGGAGGCGGCCCGTGTGGCGGAGCAGCTGAGAGC
CTACCTGGAGGGCGAGTTCGTGGAGTGGCTCCGCAGATACCTGGAGAACGGGAAGGAGACGCTGCAGCGC
GCGTCAGACCCCCCCAAGACACATATGACCCACTACCCCATCTCTGACCATGAGGCCACCCTGAGGTGCT
GGGCCCTGGGCTTCTACCCTGCGGAGATCACACTGACCTGGCAGCGGGATGGGGAGGACCAGACCACGGA
GCTCGTGGAGACCAGGCCTCCAGGGGATGGAACCTTCCAGAAGTGGGCGGCTGTGGTGGTGCCTTCTGGA
GAGGAGCAGAGATACACCTGCCATGTGCAGCATGAGGGTCTGCCCGAGCCCCTCACCCTGAGATGGCAGG
GTCAGGGTCCCTCACCTTCCCCCCTTTTCCCAGACCCATCTTCCCAGCCCACCATCCCCATCGTGGGCAT
CATTGCTGGCCTGGTTCTACTTGTAGCTGTGGTCACTGGAGCTGTGGTCACTGCTGTAATGTGGAGGAAG
AAGAGCTCAGGTAAGGAAGGGGATGGGTATTCTACTCCAGGCGGCAACAGTGCCCAGGGCTCTGATGTGT
CTCTCACGGCGTGAAAGGTGAGACCTTGGGGGGCCTGAT

In a search of public sequence databases, the NOV31 nucleic acid sequence, located on chromsome 6 has 404 of 512 bases (78%) identical to a gb:GENBANK-ID:AF055066|acc:AF055066.1 mRNA from Homo sapiens (Homo sapiens MHC class I region). Public nucleotide databases include all GenBank databases and the GeneSeq patent database.

The disclosed NOV31 polypeptide (SEQ ID NO:72) encoded by SEQ ID NO:71 has 371 amino acid residues and is presented in Table 31B using the one-letter amino acid code. Signal P, Psort and/or Hydropathy results predict that NOV31 has a signal peptide and is likely to be localized at the plasma membrane with a certainty of 0.7300. The most likely cleavage site for a NOV31 peptide is between amino acids 18 and 19.

TABLE 31B
Encoded NOV31 protein sequence.

| | |
|---|---|
| MVLMAPRTLLLLLSGALTQTWARSHSMRYFYTTMSRPGRGEPRFISVGYVDYTQFVRFDS | (SEQ ID NO:72) |
| DDASPREEPRAPWMEREGPEYWDRNTQICKAQARTERENLRIALRYYNQSEGGGSHTMQV | |
| MYGCDVGPDGRFLRGYEQHAYDGKDYIALNEDLRSWTAADMAAQITKRKWEAARVAEQLR | |
| AYLEGEFVEWLRRYLENGKETLQRASDPPKTHMTHYPISDHEATLRCWALGFYPAEITLT | |
| WQRDGEDQTTELVETRPAGDGTFQKWAAVVVPSGEEQRYTCHVQHEGLPEPLTLRWQGQG | |
| PSPSPLFPEPSSQPTIPIVGIIAGLVLLVAVVTGAVVTAVMWRKKSSGKEGDGYSTPGGN | |
| SAQGSDVSLTA | |

A search of sequence databases reveals that the NOV31 amino acid sequence has 335/368 (91%) identity and 342/368 (92%) similarity with REMTREMBL-ACC:CAB66931 Gogo-H protein—Gorilla gorilla (gorilla). Public amino acid databases include the GenBank databases, SwissProt, PDB and PIR.

NOV31 is expressed in at least Bone Marrow, Dermis, Hippocampus, Placenta, Tonsils. This information was derived by determining the tissue sources of the sequences that were included in the invention including but not limited to SeqCalling sources, Public EST sources, Literature sources, and/or RACE sources.

The disclosed NOV31 polypeptide has homology to the amino acid sequences shown in the BLASTP data listed in Table 31C.

TABLE 31C
BLAST results for NOV31

| Gene Index/ | | Length | Identity | Positives | |
|---|---|---|---|---|---|
| Identifier | Protein/Organism | (aa) | (%) | (%) | Expect |
| gi\|232260\|sp\|P01893\| | HLA class I | 362 | 330/376 | 339/376 | e−167 |
| HLAH_HUMAN | histocompatibility | | (87%) | (89%) | |
| | antigen, alpha | | | | |
| | chain H precursor | | | | |
| | (HLA-AR) (HLA- | | | | |
| | 12.4) | | | | |
| gi\|70075\|pir\|\|HLHU12 | MHC class I | 359 | 329/373 | 337/373 | e−166 |
| | histocompatibility | | (88%) | (90%), | |
| | antigen HLA | | | | |
| | alpha chain | | | | |
| | precursor (clone | | | | |
| | pHLA 12.4) - | | | | |
| | human | | | | |
| gi\|2118771\|pir\|\|I54493 | MHC class I | 365 | 303/376 | 326/376 | e−164 |
| | histocompatibility | | (80%) | (86%), | |
| | antigen HLA-A | | | | |
| | alpha chain | | | | |
| | precursor - | | | | |
| | human | | | | |
| gi\|4164602\|gb\|AAD05568.1\| | MHC class I | 365 | 302/376 | 325/376 | e−163 |
| (AF116214) | antigen heavy | | (80%) | (86%), | |
| | chain [Homo | | | | |
| | sapiens] | | | | |
| gi\|915219\|gb\|AAA73518.1\| | MHC class I | 365 | 302/376 | 326/376 | e−163 |
| (U25971) | antigen HLA-A2407 | | (80%) | (86%), | |
| | [Homo sapiens] | | | | |

Tables 31D-E list the domain descriptions from DOMAIN analysis results against NOV31. This indicates that the NOV31 sequence has properties similar to those of other proteins known to contain this domain.

TABLE 31D
Domain Analysis of NOV31

gnl|Pfam|pfam00129, MHC_I, Class I Histocompatibility antigen,
domains alpha 1 and 2

CD-Length = 179 residues, 99.4% aligned

Score = 284 bits (727), Expect = 5e−78

TABLE 31E
Domain Analysis of NOV31

gnl|Smart|smart00407, IGc1, Immunoglobulin C-Type

CD-Length = 75 residues, 98.7% aligned

Score = 77.8 bits (190), Expect = 1e−15

The disclosed NOV31 nucleic acid of the invention encoding a MHC Class I antigen-like protein includes the nucleic acid whose sequence is provided in Table 31A or a fragment thereof. The invention also includes a mutant or variant nucleic acid any of whose bases may be changed from the corresponding base shown in Table 31A while still encoding a protein that maintains its MHC Class I antigen-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 22 percent of the bases may be so changed.

The disclosed NOV31 protein of the invention includes the MHC Class I antigen-like protein whose sequence is provided in Table 31B. The invention also includes a mutant or variant protein any of whose residues may be changed from the corresponding residue shown in Table 31B while still encoding a protein that maintains its MHC Class I antigen-like activities and physiological functions, or a functional fragment thereof. In the mutant or variant protein, up to about 9 percent of the residues may be so changed.

The invention further encompasses antibodies and antibody fragments, such as F_{ab} or (F_{ab})₂, that bind immunospecifically to any of the proteins of the invention.

The above defined information for this invention suggests that this MHC Class I antigen-like protein (NOV31) may function as a member of a “MHC Class I antigen family”. Therefore, the NOV31 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV31 nucleic acids and proteins of the invention are useful in potential therapeutic applications implicated in diseases including but not limited to various pathologies and disorders as indicated below. For example, a cDNA encoding the MHC Class I antigen-like protein (NOV31) may be useful in gene therapy, and the MHC Class I antigen-like protein (NOV31) may be useful when administered to a subject in need thereof. By way of nonlimiting example, the compositions of the present invention will have efficacy for treatment of patients suffering from Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Tonsilitis, Hemophilia, hypercoagulation, Idiopathic thrombocytopenic purpura, autoimmume disease, allergies, immunodeficiencies, transplantation, Graft vesus host as well, or other pathologies or conditions. The NOV nucleic acid encoding the MHC Class I antigen-like protein of the invention, or fragments thereof, may further be useful in diagnostic applications, wherein the presence or amount of the nucleic acid or the protein are to be assessed.

NOV31 nucleic acids and polypeptides are further useful in the generation of antibodies that bind immuno-specifically to the novel NOV31 substances for use in therapeutic or diagnostic methods. These antibodies may be generated according to methods known in the art, using prediction from hydrophobicity charts, as described in the “Anti-NOVX Antibodies” section below. The disclosed NOV31 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV32

A disclosed NOV32 nucleic acid of nucleotides (also referred to as CG57660-01) encoding a retinoic acid receptor responder-like protein is shown in Table 32A. The start and stop codons are in bold letters.

TABLE 32A
NOV32 nucleotide sequence.

(SEQ ID NO:73)

AGGGGAAGCATGAGACGGCTGCGGATCTCCCTCGCCCCGTGGGTGGGCGCGGGGGACGCGGGAGGGGCCG
AGCTCACGGGGCCAGCGCCGGGGCCTGCAGGTGGCCCTGGAGGAATCTGCAAGCACCCGCCCGTGCAGCG
GGCCTTCCGGGAGACCAGTGTGGACAGCGCCCTGGACACGCCCTTCCCAGCTGGAACATCTGTGAGGCTG
GAATTTAAGCTCCGGCAGACAAGCGGCTGGAGGAAGGCCTGGAAGAAACCCAAGTGCAAAGCCCAGCCCG
AGAGGAGGAAACAGAAATGCCTGACCTGCGTCAAAATGGACTGTGAGGATAAGGTTCTGGGCAGGATGGT
TCGCTGCCCTCCAGAGACGCAGACTCGGCGGGAGCCTGAGGACCACCAGGGGGCCGGGTGCAGCCCGGCG
GAGCGGGCGGGGAGGACCCCACGGCGGAGCGGGCGGGGAGGACCCCACCGCTGCCGCTTCCCTGCACGGT
TCGCCTCCTCCAAGGCCCGGCCCCCACCGGAGCCCTAGCGCTGAATCGCATGGCGCCCCCTGGAGCCCTG
GCGGG

In a search of public sequence databases, the NOV32 nucleic acid sequence, located on chromsome 4 has 443 of 451 bases (98%) identical to a gb:GENBANK-ID:AF146191|acc:AF146191.1 mRNA from Homo sapiens (Homo sapiens FRG1 (FRG1) gene, complete cds; 5S ribosomal RNA gene, complete sequence; TUB4q and TIG2 pseudogenes, complete sequence). Public nucleotide databases include all GenBank databases and the GeneSeq patent database.

The disclosed NOV32 polypeptide (SEQ ID NO:74) encoded by SEQ ID NO:73 has amino acid residues and is presented in Table 32B using the one-letter amino acid code. Signal P, Psort and/or Hydropathy results predict that NOV32 has a signal peptide and is likely to be localized at the plasma membrane with a certainty of 0.8800.

TABLE 32B
Encoded NOV32 protein sequence.

| | |
|---|---|
| MRRLRISLAPWVGAGDAGGAELTGPAPGPAGGPGGICKHPPVQRAFRETSVDSALDTPFP | (SEQ ID NO:74) |
| ACTSVRLEFKLRQTSGWRKAWKKPKCKAQPERRKQKCLTCVKMDCEDKVLGRMVRCPPET | |
| QTRREPEEHQGAGCSPAERAGRTPRRSGRGGPHGCRFPARFASSKARPPAEP | |

A search of sequence databases reveals that the NOV32 amino acid sequence has 94/168 (55%) identity and 109/168 (64%) similarity with ptnr:SWISSPROT-ACC:Q99969 RETINOIC ACID RECEPTOR RESPONDER PROTEIN 2 PRECURSOR (TAZAROTENE-INDUCED GENE 2 PROTEIN) (RAR-RESPONSIVE PROTEIN TIG2)—Homo sapiens. Public amino acid databases include the GenBank databases, SwissProt, PDB and PIR.

NOV32 is expressed in at least Adipose, Adrenal gland, Breast, Colon, Esophagus, Eye, Heart, Kidney, Liver, Lung, Ovary, Parathyroid, Placenta, Prostate, Stomach, Testis, Uterus, Whole embryo, bladder tumor, brain, cervix, colon, head and neck, kidney, lung, muscle and ovary. This information was derived by determining the tissue sources of the sequences that were included in the invention including but not limited to SeqCalling sources, Public EST sources, Literature sources, and/or RACE sources.

The disclosed NOV32 polypeptide has homology to the amino acid sequences shown in the BLASTP data listed in Table 32C.

TABLE 32C
BLAST results for NOV32

| Gene Index/ | | Length | Identity | Positives | |
|---|---|---|---|---|---|
| Identifier | Protein/Organism | (aa) | (%) | (%) | Expect |
| gi\|4506427\|ref\|NP_002880.1\| | retinoic acid | 163 | 94/169 | 109/169 | 8e−37 |
| (NM_002889) | receptor | | (55%) | (63%) | |
| | responder | | | | |
| | (tazarotene | | | | |
| | induced) 2 [Homo | | | | |
| | sapiens] | | | | |
| gi\|12832179\|dbj\|BAB21997.1\| | homolog to | 162 | 72/169 | 94/169 | 3e−22 |
| (AK002298) | RETINOIC ACID | | (42%) | (55%), | |
| | RECEPTOR | | | | |
| | RESPONDER PROTEIN | | | | |
| | 2 PRECURSOR | | | | |
| | (TAZAROTENE- | | | | |
| | INDUCED GENE 2 | | | | |
| | PROTEIN) (RAR- | | | | |
| | RESPONSIVE | | | | |
| | PROTEIN | | | | |
| | TIG2)˜putative | | | | |
| | [Mus musculus] | | | | |
| gi\|17436162\|ref\|XP_ | similar to | 206 | 73/74 | 74/74 | 1e−20 |
| 067978.1\| | retinoic acid | | (98%) | (99%) | |
| (XM_067978) | receptor | | | | |
| | responder | | | | |
| | (tazarotene | | | | |
| | induced) 2 [Homo | | | | |
| | sapiens] | | | | |
| gi\|18585954\|ref\|XP_ | hypothetical | 322 | 52/79 | 59/79 | 2e−18 |
| 091375.1\| | protein XP_091375 | | (65%) | (73%) | |
| (XM_091375) | [Homo sapiens] | | | | |
| gi\|17488051\|ref\|XP_ | similar to | 296 | 28/54 | 32/54 | 2e−5 |
| 064055.1\| | Fibroblast growth | | (51%) | (58%), | |
| (XM_064055) | factor receptor 3 | | | | |
| | precursor (FGFR- | | | | |
| | 3) (Heparin- | | | | |
| | binding growth | | | | |
| | factor receptor) | | | | |
| | [Homo sapiens] | | | | |

Retinoids exert their biologic effects through two families of nuclear receptors, retinoic acid receptors (RARs) and retinoid X receptors (RXRs), which belong to the superfamily of steroid/thyroid hormone nuclear receptors. The retinoid-mediated up-regulation in the expression of TIG2 was confirmed by Northern blot analysis. Upon sequencing, TIG2 was found to be a cDNA whose complete sequence was not in the GenBank and EMBL data bases. The TIG2 cDNA is 830 bp long and encodes a putative protein product of 164 amino acids. TIG2 is neither expressed nor induced by tazarotene in primary keratinocyte and fibroblast cultures. Thus, TIG2 is expressed and induced by tazarotene only when keratinocytes and fibroblasts form a tissue-like 3-dimensional structure. RAR-specific retinoids increase TIG2 mRNA levels. In contrast, neither RXR-specific retinoids nor 1,25-dihydroxyvitamin D3 increased TIG2 levels. TIG2 is expressed at high levels in nonlesional psoriatic skin but at lower levels in the psoriatic lesion and that its expression is up-regulated in psoriatic lesions after topical application of tazarotene.

The disclosed NOV32 nucleic acid of the invention encoding a retinoic acid receptor responder-like protein includes the nucleic acid whose sequence is provided in Table 32A or a fragment thereof. The invention also includes a mutant or variant nucleic acid any of whose bases may be changed from the corresponding base shown in Table 32A while still encoding a protein that maintains its retinoic acid receptor responder-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2 percent of the bases may be so changed.

The disclosed NOV32 protein of the invention includes the retinoic acid receptor responder-like protein whose sequence is provided in Table 32B. The invention also includes a mutant or variant protein any of whose residues may be changed from the corresponding residue shown in Table 32B while still encoding a protein that maintains its retinoic acid receptor responder-like activities and physiological functions, or a functional fragment thereof. In the mutant or variant protein, up to about 45 percent of the residues may be so changed.

The invention further encompasses antibodies and antibody fragments, such as F_{ab} or (F_{ab})₂, that bind immunospecifically to any of the proteins of the invention.

The above defined information for this invention suggests that this retinoic acid receptor responder-like protein (NOV32) may function as a member of a “retinoic acid receptor responder family”. Therefore, the NOV32 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV32 nucleic acids and proteins of the invention are useful in potential therapeutic applications implicated in diseases including but not limited to various pathologies and disorders as indicated below. For example, a cDNA encoding the retinoic acid receptor responder-like protein (NOV32) may be useful in gene therapy, and the retinoic acid receptor responder-like protein (NOV32) may be useful when administered to a subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Obesity, Endometriosis, Fertility, Hemophilia, Hypercoagulation, Idiopathic thrombocytopenic purpura, Immunodeficiencies, Graft vesus host, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, or other pathologies or conditions. The NOV32 nucleic acid encoding the retinoic acid receptor responder-like protein of the invention, or fragments thereof, may further be useful in diagnostic applications, wherein the presence or amount of the nucleic acid or the protein are to be assessed.

NOV32 nucleic acids and polypeptides are further useful in the generation of antibodies that bind immuno-specifically to the novel NOV32 substances for use in therapeutic or diagnostic methods. These antibodies may be generated according to methods known in the art, using prediction from hydrophobicity charts, as described in the “Anti-NOVX Antibodies” section below. The disclosed NOV32 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV33

A disclosed NOV33 nucleic acid of 1706 nucleotides (also referred to as) encoding a PHOSPHATIDYLINOSITOL 4-PHOSPHATE 5-KINASE-like protein is shown in Table 33A. The start and stop codons are in bold letters.

TABLE 33A
NOV33 nucleotide sequence.

(SEQ ID NO:75)

CTGCCAAGATGGCGTCGGCCTCCTCCCAACCGTCGTTGGCGGTCGGTTTTTCATCCTTTGATCCCGGGCC
CCCTTCCTGTACCGCGTCCTCAGCATCTGGAATCTTCAGCCCCACGGCATCTGAGGTGCCTTATCCCTCT
GGCATGCCCATCAAGAAAACAGGCCATCGAGGTGTCGATTCCTCAGGAGAGACAACATATAAAAAGACAA
CCTCAACAGCCTTGAAAGGTGCCATCCAGTTAGGCATTACTTACACTGTGGGGAGCCTGAGTACCAAACC
AGAGCGTGATGTCCTCATGCAAGATTTCTACGTGGTGGAGAGTATCTTCTTCCCCAGTGAAGGGAGCAAC
CTGACCCCTGCTCATCACTACAATGCCTTCCGTTTCAAGACCTATGCGCCGGTTGCCTTCCGCTACTTTC
GGGAGCTATTTGGTATCCCGCCCGATGATTACTTGTGCTCCCTCTGCAGTGAGCCGCTGATTCAACTCTG
TAGCTCTGGAGCTAGTGGTTCCCTGTTCTATGTGTCCAGCGACGATGAACTCATTATTAAGACACTCCAA
CATAAAGAGGCGGAGTTTCTGCAGAAGCTGCTTCCAGGATACTACTTGAACCTCAGCCAGAACCCTCGGA
CTTTGCTCCCTAAATTCTTTGCACTGTACTGTGTGCAGACAGGTGCCAAGAACATTCGGATTGTGGTGAT
GAACAATCTTTTACCAAGATCCGTCAAAATGCATATCAAATATGACCTCAAAGGCTCAACCTACAAACGC
CGGGCTTCCCAGAAAGAGCGAGAGAAGCCTCTTCCCACATTTAAAGATCTAGACTTCTTACAAGACATCC
CTGATGGTCTTTTTTTGGATGCTGACACGTACAATGCTCTCTGTAAGACCCTGCAGCGTGACTGTTTGGT
GCTGCAGAGCTTCAAGATAATGGACTATAGCCTCTGGCTGTCAATCCACAATATAGATCATGCACAACGA
GAGCCCTTAAGCAGCGACACTCTTCAAGTGTCAATCGACACTCAAAGACTGGCTCCCCAAAAGGCTCTGT
ATTCCACAGCCATGGAATTCATCCAGGCACACGCTCGGCTGGGCGACACCATGGAGGCCGATGACCATAT
GGGTGGCATCCCTGCTCAGAATAGTAAAGGGGAAAGGCTTCTGCTTTATATTGGCATCATTGACATTCTA
CAGTCTTACACGTTTCTTAAGAAGTTGGAGCACTCTTCGAAAGCCGTGGTACATGATGCGGACGCTGTCT
CAGTGCATCGCCCAGGCTTCTACGCTGAACGGTTCCAGCACTTCATGTGCAACGCAGTATTTAAGAAGAT
CCCCTTGAAGCCTTCTCCTTCCAAAAAGTTTCGGTCTGGCTTATCTTTCTCTCTGCATACGGGCTCCAGT
GGCAACTCCTGCATTACTTACCAGCCATTGGTCTCTGAGGAACACAAGTCACAAGTGATAAAGGTGCAAG
TGGAGCCAGGTGTTCACCTTGGTCGTTCTGATGTTTTACCTCAGACCTCAGAATCCACCTTTGGAGGAAA
TCAGGGAGGGCTCACTATTACTGACCACAGTTTCTCACCTGTAGTTGGAAAGACTTTGCATATGCTAACT
ACAAGTATAACCTTGGAAAAACTTGAATGTACAGAGTCAGAGTTCACCCATTAAGCGCAAAGCCTCAGAA
GACCTGGAACAAGATTCTGCTTCTCT

In a search of public sequence databases, the NOV33 nucleic acid sequence, located on chromsome 6 has 1586 of 1706 bases (92%) identical to a gb:GENBANK-ID:HSU78575|acc:U78575.11 mRNA from Homo sapiens (Human 68 kDa type I phosphatidylinositol-4-phosphate 5-kinase alpha mRNA, clone PIP5KIa1, complete cds). Public nucleotide databases include all GenBank databases and the GeneSeq patent database.

The disclosed NOV33 polypeptide (SEQ ID NO:76) encoded by SEQ ID NO:75 has 551 amino acid residues and is presented in Table 33B using the one-letter amino acid code.

TABLE 33B
Encoded NOV33 protein sequence.

| | |
|---|---|
| MASASSQPSLAVGFSSFDPGAPSCTASSASGILSPTASEVPYASGMPIKKTGHRGVDSSG | (SEQ ID NO:76) |
| ETTYKKTTSTALKGAIQLGITYTVGSLSTKPERDVLMQDFYVVESIFFPSEGSNLTPAHH | |
| YNAFRFKTYAPVAFRYFRELFGIPPDDYLCSLCSEPLIELCSSGASGSLFYVSSDDELII | |
| KTLQHKEAEFLQKLLPGYYLNLSQNPRTLLPKFFGLYCVQTGGKNIRIVVMNNLLPRSVK | |
| MHIKYDLKGSTYKRRASQKEREKPLPTFKDLDFLQDIPDGLFLDADTYNALCKTLQRDCL | |
| VLQSFKIMDYSLWLSIHNIDHAQREPLSSDTLQVSIDTQRLAPQKALYSTANEFIQGEAR | |
| LGDTMEADDHMGGIPAQNSKGERLLLYIGIIDILQSYTFLKKLEHSWKAVVHDGDAVSVH | |
| RPGFYAERFQHFMCNAVFKKIPLKPSPSKKFRSGLSFSLHTGSSGNSCITYQPLVSEEHK | |
| SQVIKVQVEPGVHLGRSDVLPQTSESTFGGNQGGLTITDHSFSPVVGKTLHMLTTSITLE | |
| KLECTESEFTH | |

A search of sequence databases reveals that the NOV33 amino acid sequence has 478 of 551 amino acid residues (86%) identical to, and 496 of 551 amino acid residues (90%) similar to, the 549 amino acid residue ptnr:SPTREMBL-ACC:Q99754 protein from Homo sapiens (Human) (68 KDA TYPE I PHOSPHATIDYLINOSITOL-4-PHOSPHATE 5-KINASE ALPHA (EC 2.7.1.68) (1-PHOSPHATIDYLINOSITOL-4-PHOSPHATE KINASE) (DIPHOSPHOINOSITIDE KINASE) (PTDINS(4)P-5-KINASE)). Public amino acid databases include the GenBank databases, SwissProt, PDB and PIR.

NOV33 is expressed in at least Adrenal gland, Aorta, B-cells, Blood, Bone, Brain, Breast, CNS, Colon, Ear, Esophagus, Eye, Gall bladder, Germ Cell, Head and neck, heart, Kidney, Larynx, Liver, Lung, Lymph, Marrow, Muscle, Neural, Omentum, Ovary, Pancreas, Parathyroid, Peripheral nervous system, Placenta, Pooled, Prostate, Skin, Small intestine, Spleen, Stomach, Synovial membrane, Testis, Tissue culture, Tonsil, Uterus, Whole embryo, and adrenal gland. Expression information was derived from the tissue sources of the sequences that were included in the derivation of the sequence of CG57672-01. The sequence is predicted to be expressed in the following tissues because of the expression pattern of (GENBANK-ID: gb:GENBANK-ID:HSU78575|acc:U78575.1) a closely related Human 68 kDa type I phosphatidylinositol-4-phosphate 5-kinase alpha mRNA, clone PIP5KIa1, complete cds homolog in species Homo sapiens: fetal brain. This information was derived by determining the tissue sources of the sequences that were included in the invention including but not limited to SeqCalling sources, Public EST sources, Literature sources, and/or RACE sources.

The disclosed NOV33 polypeptide has homology to the amino acid sequences shown in the BLASTP data listed in Table 33C.

TABLE 33C
BLAST results for NOV33

| Gene Index/ | | Length | Identity | Positives | |
|---|---|---|---|---|---|
| Identifier | Protein/Organism | (aa) | (%) | (%) | Expect |
| gi\|4505815\|ref\|NP_003548.1\| | phosphatidylinositol- | 549 | 478/552 | 496/552 | 0.0 |
| (NM_003557) | 4-phosphate | | (86%) | (89%), | |
| | 5-kinase, type I, | | | | |
| | alpha [Homo | | | | |
| | sapiens] | | | | |
| gi\|1743873\|gb\|AAC50911.1\| | 68 kDa type I | 562 | 478/565 | 496/565 | 0.0 |
| (U78576) | phosphatidylinositol- | | (84%) | (87%), | |
| | 4-phosphate | | | | |
| | 5-kinase alpha | | | | |
| | [Homo sapiens] | | | | |
| gi\|1743875\|gb\|AAC50912.1\| | 68 kDa type I | 500 | 439/551 | 455/551 | 0.0 |
| (U78577) | phosphatidylinositol- | | (79%) | (81%), | |
| | 4-phosphate | | | | |
| | 5-kinase alpha | | | | |
| | [Homo sapiens] | | | | |
| gi\|6679331\|ref\|NP_032873.1\| | phosphatidylinositol- | 546 | 434/552 | 470/552 | 0.0 |
| (NM_008847) | 4-phosphate | | (78%) | (84%), | |
| | 5-kinase, type 1 | | | | |
| | beta; PI4P5K-I[b] | | | | |
| | [Mus musculus] | | | | |
| gi\|14745097\|ref\|XP_ | similar to | 435 | 361/408 | 381/408 | 0.0 |
| 018166.2\| | phosphatidylinositol- | | (88%) | (92%), | |
| (XM_018166) | 4-phosphate | | | | |
| | 5-kinase, type I, | | | | |
| | alpha (H. | | | | |
| | sapiens) [Homo | | | | |
| | sapiens] | | | | |

Tables 33D-E list the domain descriptions from DOMAIN analysis results against NOV33. This indicates that the NOV33 sequence has properties similar to those of other proteins known to contain this domain.

TABLE 33D
Domain Analysis of NOV33

gnl|Smart|smart00330, PIPKc, Phosphatidylinositol phosphate kinases

CD-Length = 339 residues, 100.0% aligned

Score = 410 bits (1054), Expect = 1e−115

TABLE 33E
Domain Analysis of NOV33

gnl|Pfam|pfam01504, PIP5K, Phosphatidylinositol-4-phosphate 5-Kinase.
This family contains a region from the common kinase core found in the
type I phosphatidylinositol-4-phosphate 5-kinase (PIP5K) family as
described in. The family consists of various type I, II and III PIP5K
enzymes. PIP5K catalyses the formation of phosphoinositol-4,5-
bisphosphate via the phosphorylation of phosphatidylinositol-4-
phosphate a precursor in the phosphinositide signaling pathway

CD-Length = 293 residues, 99.7% aligned

Score = 390 bits (1002), Expect = 1e−109

Phosphatidylinositol-4-phosphate 5-kinases (P1P5K) synthesize phosphatidylinositol-4,5-bisphosphate, a key precursor in phosphoinositide signaling that also regulates some proteins and cellular processes directly. Two distinct PIP5Ks have been characterized in erythrocytes, the 68-kDa type I (PIP5KI) and 53-kDa type II (PIP5KII) isoforms. Using peptide sequences from the erythroid 68-kDa PIP5KI, cDNAs encoding PIP5KI from human brain were isolated. Partial cDNAs obtained for a second isoform, PIP5KI, established that the human STM7 gene encoded a previously unrecognized PIP5KI. However, the peptide sequences demonstrated that erythroid PIP5KI corresponded to PIP5KI. Recombinant, bacterially expressed PIP5KI possessed PIP5K activity and was immunoreactive with erythroid PIP5KI antibodies. By Northern analysis, PIP5KI and PIP5KI had wide tissue distributions, but their expression levels differed greatly. PIP5KIs had homology to the kinase domains of PIP5KII, yeast Mss4p and Fab1p, and a new Caenorhabditis elegans Fab1-like protein identified in the data base. These new isoforms have refined the sequence requirements for PIP5K activity and, potentially, regulation of these enzymes. Furthermore, the limited homology between PIP5KIs and PIP5KII, which was almost exclusively within the kinase domain core, provided a molecular basis for distinction between type I and II PIP5Ks. Phosphatidylinositol-4-phosphate 5-kinases (PIP5Ks) synthesize phosphatidylinositol 4,5-bisphosphate by phosphorylating phosphatidylinositol 4-phosphate. By searching sequence databases with peptide sequences obtained from the 68-kD type I PIP5K purified from bovine erythrocytes, Loijens and Anderson (1996) identified a human EST encoding PIP5K1A, which they called PIP5KI-alpha. They screened a human fetal brain cDNA library and isolated full-length PIP5K1A cDNAs. The deduced 549-amino acid protein has the conserved kinase homology domain of PIP5K family members. Within this domain, 131P5K1A shows 83% and 35% amino acid identity with PIP5K1B and PIP5K2A, respectively. Overall, the PIP5K1A and PIP5K1B proteins are 64% identical. Recombinant PIP5K1A expressed in bacteria had a molecular mass of approximately 66.3 kD by Western blot analysis. The authors isolated additional PIP5K1A cDNAs which they suggested represent splicing isoforms. Northern blot analysis detected a major 4.2-kb PIP5K1A transcript which had a wide tissue distribution.

The disclosed NOV33 nucleic acid of the invention encoding a PHOSPHATIDYLINOSITOL 4-PHOSPHATE 5-KINASE-like protein includes the nucleic acid whose sequence is provided in Table 33A or a fragment thereof. The invention also includes a mutant or variant nucleic acid any of whose bases may be changed from the corresponding base shown in Table 33A while still encoding a protein that maintains its PHOSPHATIDYLINOSITOL 4-PHOSPHATE 5-KINASE-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 8 percent of the bases may be so changed.

The disclosed NOV33 protein of the invention includes the PHOSPHATIDYLINOSITOL 4-PHOSPHATE 5-KINASE-like protein whose sequence is provided in Table 33B. The invention also includes a mutant or variant protein any of whose residues may be changed from the corresponding residue shown in Table 33B while still encoding a protein that maintains its PHOSPHATIDYLINOSITOL 4-PHOSPHATE 5-KINASE-like activities and physiological functions, or a functional fragment thereof. In the mutant or variant protein, up to about 14 percent of the residues may be so changed.

The invention further encompasses antibodies and antibody fragments, such as F_{ab} or (F_{ab})₂, that bind immunospecifically to any of the proteins of the invention.

The above defined information for this invention suggests that this PHOSPHATIDYLINOSITOL 4-PHOSPHATE 5-KINASE-like protein (NOV33) may function as a member of a “PHOSPHATIDYLINOSITOL 4-PHOSPHATE 5-KINASE family”. Therefore, the NOV33 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV33 nucleic acids and proteins of the invention are useful in potential therapeutic applications implicated in diseases including but not limited to various pathologies and disorders as indicated below. For example, a cDNA encoding the PHOSPHATIDYLINOSITOL 4-PHOSPHATE 5-KINASE-like protein (NOV33) may be useful in gene therapy, and the PHOSPHATIDYLINOSITOL 4-PHOSPHATE 5-KINASE-like protein (NOV33) may be useful when administered to a subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Adrenoleukodystrophy, Congenital Adrenal Hyperplasia, Hemophilia, Hypercoagulation, Idiopathic thrombocytopenic purpura, Immunodeficiencies, Graft vesus host, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, or other pathologies or conditions. The NOV33 nucleic acid encoding the PHOSPHATIDYLINOSITOL 4-PHOSPHATE 5-KINASE-like protein of the invention, or fragments thereof, may further be useful in diagnostic applications, wherein the presence or amount of the nucleic acid or the protein are to be assessed.

NOV33 nucleic acids and polypeptides are further useful in the generation of antibodies that bind immuno-specifically to the novel NOV33 substances for use in therapeutic or diagnostic methods. These antibodies may be generated according to methods known in the art, using prediction from hydrophobicity charts, as described in the “Anti-NOVX Antibodies” section below. The disclosed NOV33 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV34

A disclosed NOV34 nucleic acid of 1316 nucleotides (also referred to as CG57680-01) encoding a Cyclophilin-type peptidyl-prolyl cis-trans isomerase-like protein is shown in Table 34A. The start and stop codons are in bold letters.

TABLE 34A
NOV34 nucleotide sequence.

(SEQ ID NO:77)

TCGGTGCCATGGCGGTTCTACTGGAGACTACTGTGGGCAATGTGGTTGTCAATTTGCACACTGAGCAGCA
GCCTTGCAACTGTGAACTTTTTGAGAGCAGGTACCACAGTTTAATGGCATTTAATTTCTTGAGATATTAC
AAAATAAAATATTACAGTTATTGCCTTATTCACAGTATACAAAGGTATTTTATCATACAAACTGTTGATC
CTACAGGGACTGGTCATGGAGGAGAGTCTATTTTTGGCCTAGGATTGTATGGTGATCAAGCAAGCTTTTT
TGAGACAGAAAACGTCCCAAGAATTAAGCACAAGAAGAAGGGCACAATGTCCATGGTGAATAATGACACT
GATCAACATGGATCTCAGTTTCTTATCACTACAGGAGAAAATCTAGATTACCTTCATGGTACCCATACAG
TATTTGGTGAGGTGACAGAAGGCATTGACATAATTAAGAAAATAAATGAGACCTTTGTTGACAAGGACTT
TGTACCATATCAGGATATCACGATAAATTATATAGTCATTTTACATCCTCCATTTGATGACATTCCTGAT
TTATTAATCCCTGATCAATCACCACAACCTACAAGGGAACAATTAAAGAGTGGTAGAGTTGACACAAATG
AAGAAATTGATCATTTCAAACGAAGGTCAGCCGAAGAAGTAGAAGAAATAAAGGCAGAAAAAGAAGCTAA
AACTCAGGCTTTACTTTTAGAGATGGTGGGAGACCTACCTGATGCAGATATTAAACCTCCGGAAAAATCT
GTGTTTGTATGCAAATTGAATCCAGTGACCACAGATGAGGATCTGGATATAATACTCTCTAGATTTGGGC
CAATAAGAAGTTGTGAAGTTATCTGGGACTGGAAGACAGGAGAAATCCTCTGTTATTTCTTTCTTTCTTT
CTATGCTTTTATTGAATTTGAAAAGGAAGAAGATTATGAGAAAGCCTTCTTCAAAATGGACAATATACTT
ATAGATGACAGAAGAAAACATGCATTTCCCACTCTGTTACAAAGGTTAAATGGAAGGAAAAAGTGGGAAA
TACACCAACACCCGGGGCGCAGCCCACGCCGCCGCCGCCGCCCGCACCGTTCCCGCTCCCGGCGGCGGCG
ACGGCGGGCGGGGACCCCGCGGCGCTGCGCCTCATCCTCTGCGACGGGGGATGAGGGGTCTGAGAGGAAC
TGGAGGAGGAGGAGGAGGAGGCAGTGGACATGGTGGCTCTGCGGGCCCTGGACGCCGCCGGCCGACACGG
AGGCGCGGGGCGGGAGGCGCGCGAGCGTGTGGGAGCCGGCCCGATAGTGCCTCCGA

In a search of public sequence databases, the NOV34 nucleic acid sequence, located on chromsome 7 has 149 of 235 bases (63%) identical to a gb:GENBANK-ID:A45258|acc:A45258.1 mRNA from human herpesvirus 2 (Sequence 2 from Patent WO9516779). Public nucleotide databases include all GenBank databases and the GeneSeq patent database.

The disclosed NOV34 polypeptide (SEQ ID NO:78) encoded by SEQ ID NO:77 has 432 amino acid residues and is presented in Table 34B using the one-letter amino acid code. Signal P, Psort and/or Hydropathy results predict that NOV34 has a signal peptide and is likely to be localized at the plasma membrane with a certainty of 0.9820.

TABLE 34B
Encoded NOV34 protein sequence.

| | |
|---|---|
| MAVLLETTVCNVVVNLHTEQQPCNCELFESRYHSLMAFNFLRYYKIKYYSYCLIHSIQRY | (SEQ ID NO:78) |
| FIIQTVDPTGTGNGGESIFGLGLYCDQASFFETENVPRIKHKKKGTMSMVNNDSDQHGSQ | |
| FLITTGENLDYLDGTHTVFGEVTEGIDIIKKINETFVDKDFVPYQDIRINYIVILDGPFD | |
| DIPDLLIPDQSPEPTREQLKSGRVDTNEEIDHFKRRSAEEVEEIKAEKEAKTQALLLEMV | |
| GDLPDADIKPPEKSVFVCKLNPVTTDEDLDIILSRFGPIRSCEVIWDWKTGEILCYFFLS | |
| FYAFIEFEKEEDYEKAFFKMDNILIDDRRKHGFASLLQRLNGRKKWEIHQQPGRSPRRRR | |
| RPHRSRSRRRRRRAGTPRRCASSSATGDEGSERNWRRRRRRQWTWWLCGPWTPPADTEAR | |
| GGRRASVWEPAR | |

A search of sequence databases reveals that the NOV34 amino acid sequence has 263/345 (76%) identity and 291/345 (84%) similarity with TREMBLNEW-ACC:BAB307116 DAYS NEONATE HEAD CDNA, RIKEN FULL-LENGTH ENRICHED LIBRARY, CLONE:5430431E21, FULL INSERT SEQUENCE—Mus musculus. Public amino acid databases include the GenBank databases, SwissProt, PDB and PIR.

NOV34 is expressed in at least Adrenal Gland/Suprarenal gland, Bone, Bone Marrow, Brain, Colon, Hair Follicles, Heart, Hippocampus, Kidney, Liver, Lung, Lymphoid tissue, Pancreas, Peripheral Blood, Prostate, Salivary Glands, Small Intestine, Testis, Tonsils, Uterus. This information was derived by determining the tissue sources of the sequences that were included in the invention including but not limited to SeqCalling sources, Public EST sources, Literature sources, and/or RACE sources.

The disclosed NOV34 polypeptide has homology to the amino acid sequences shown in the BLASTP data listed in Table 34C.

TABLE 34C
BLAST results for NOV34

| Gene Index/ | | Length | Identity | Positives | |
|---|---|---|---|---|---|
| Identifier | Protein/Organism | (aa) | (%) | (%) | Expect |
| gi\|12849069\|dbj\|BAB28194.1\| | homolog to | 382 | 261/331 | 284/331 | e−138 |
| (AK012371) | DJ12G14.1 (NOVEL | | (78%) | (84%) | |
| | CYCLOPHILIN TYPE | | | | |
| | PEPTIDYL-PROLYLCIS- | | | | |
| | TRANS | | | | |
| | ISOMERASE) | | | | |
| | (FRAGMENT)˜putative | | | | |
| | [Mus musculus] | | | | |
| gi\|12847571\|dbj\|BAB27623.1\| | homolog to | 418 | 261/331 | 284/331 | e−137 |
| (AK011443) | DJ12G14.1 (NOVEL | | (78%) | (84%) | |
| | CYCLOPHILIN TYPE | | | | |
| | PEPTIDYL-PROLYLCIS- | | | | |
| | TRANS | | | | |
| | ISOMERASE) | | | | |
| | (FRAGMENT)˜putative | | | | |
| | [Mus musculus] | | | | |
| gi\|12856573\|dbj\|BAB30711.1\| | homolog to | 460 | 261/331 | 284/331 | e−136 |
| (AK017370) | DJ12G14.1 (NOVEL | | (78%) | (84%) | |
| | CYCLOPHILIN TYPE | | | | |
| | PEPTIDYL-PROLYL | | | | |
| | CIS-TRANS | | | | |
| | ISOMERASE) | | | | |
| | (FRAGMENT)˜putative | | | | |
| | [Mus musculus] | | | | |
| gi\|12852237\|dbj\|BAB29330.1\| | homolog to | 492 | 259/331 | 284/331 | e−135 |
| (AK014406) | DJ12G14.1 (NOVEL | | (78%) | (85%) | |
| | CYCLOPHILIN TYPE | | | | |
| | PEPTIDYL-PROLYL | | | | |
| | CIS-TRANS | | | | |
| | ISOMERASE) | | | | |
| | (FRAGMENT)˜putative | | | | |
| | [Mus musculus] | | | | |
| gi\|18088111\|gb\|AAH20986.1\| | (protein for | 492 | 263/331 | 284/331 | e−135 |
| AAH20986 | MGC: 9727) [Homo | | (79%) | (85%) | |
| (BC020986) | sapiens] | | | | |

Tables 34D-E list the domain descriptions from DOMAIN analysis results against NOV34. This indicates that the NOV34 sequence has properties similar to those of other proteins known to contain this domain.

TABLE 34D
Domain Analysis of NOV34

gnl|Pfam|pfam00160, pro_isomerase, Cyclophilin type peptidyl-prolyl
cis-trans isomerase

CD-Length = 162 residues, 88.9% aligned

Score = 91.7 bits (226), Expect = 8e−20

TABLE 34E
Domain Analysis of NOV34

gnl|Pfam|pfam00076, rrm, RNA recognition motif, (a.k.a. RRM, RBD, or
RNP domain). The RRM motif is probably diagnostic of an RNA binding
protein. RRMs are found in a variety of RNA binding proteins,
including various hnRNP proteins, proteins implicated in regulation of
alternative splicing, and protein components of snRNPs. The motif also
appears in a few single stranded DNA binding proteins. The RRM
structure consists of four strands and two helices arranged in an
alpha/beta sandwich, with a third helix present during RNA binding in
some cases The C-terminal beta strand (4th strand) and final helix are
hard to align and have been omitted in the SEED alignment The LA
proteins have a N terminus rrm which is included in the seed. There is
a second region towards the C terminus that has some features of a rrm
but does not appear to have the important structural core of a rrm.
The LA proteins are one of the main autoantigens in Systemic lupus
erythematosus (SLE), an autoimmune disease.
CD-Length = 71 residues, 95.8% aligned
Score = 47.8 bits (112), Expect = 1e−06

The cyclophilins are a conserved class of proteins that bind the immunosuppressive drug cyclosporin A (CsA) with high affinity. CsA blocks helper T-cell activation at a step between T-cell receptor stimulation and the transcriptional activation of cytokine genes. Cyclophilins from many species possess peptidyl-prolyl cis-trans isomerase (PPIase) activity that is blocked by CsA and therefore may be relevant in CsA-mediated immunosuppression. Probing with the previously known cyclophilin cDNA under reduced stringencies, Price et al. (1991) identified a second cyclophilin gene, which encoded cyclophilin B (CYPB). The deduced protein was 64% identical to CYPA and was distinguished from it by a signal sequence that probably directs it to the endoplasmic reticulum (OR). CYPB showed even stronger similarity to yeast CYPB, which also has an ER-directed signal sequence. The signal sequence is removed from the protein upon expression in E. coli, and the processed protein possesses PPIase activity that is inhibited by CsA. Peddada et al. (1992) used the PCR technique to generate a unique probe complementary to the hydrophobic 5-prime end of the human cyclophilin B gene. Using this probe in an analysis of human/hamster hybrid somatic cell lines, they assigned the gene to chromosome 15. The human PIN1 gene encodes an essential nuclear peptidyl-prolyl cis/trans isomerase involved in the regulation of mitosis. PIN1 is a member of a new class of peptidyl-prolyl cis/trans isomerases that includes the Escherichia coli parvulin, yeast ESS1, and Drosophila melanogaster dodo gene products. Lu et al. (1996) described human PIN1 and showed that deletion of PIN1 from HeLa cells induces mitotic arrest, while HeLa cells overexpressing PIN1 arrest in the G2 phase. Campbell et al. (1997) identified a gene closely related to the gene encoding the essential nuclear peptidyl-prolyl cis/trans isomerase (PIN1) involved in the regulation of mitosis. The novel gene, called PIN1L by them, is 89% identical at the nucleotide level to the PIN1 transcript, but contains a shift in the reading frame. It encodes a 100-amino acid variant protein consisting of 63 amino acids homologous (90% identical) to PIN1 and contains the entire WW domain, fused to a 37-amino acid tail. By fluorescence in situ hybridization and somatic cell hybrid analysis, Campbell et al. (1997) mapped PIN1L to 1q31. They commented that the protein encoded by PIN1L may have some functional role or, alternatively, PIN1L may be a transcribed pseudogene. Campbell et al. (1997) found by analysis of human expressed sequence tags (ESTs) 2 different but closely related human transcripts, 1 of which corresponds to PIN1. Gene localization, using both fluorescence in situ hybridization and tritium-labeled probes, showed that each of the human transcripts hybridized to 1p31 and 19p13. Primers were designed to discriminate between the 2 transcripts, and PCR on DNA from hamster/human somatic cell hybrids retaining chromosomes 1 or 19 was used to map the human PIN1 gene to chromosome 19, and PIN1 L, the closely related gene, to chromosome I. Their results established that PIN1 is at 19p13 and PIN1L at 1p31. PCR was used to clone the coding region for PIN1L. The PIN1L cDNA is 89% identical at the nucleotide level to the PIN1 transcript, but contained a shift in the reading frame. Campbell et al. (1997) commented that the protein encoded by PIN1L may have some functional role or, alternatively, PIN1L may be a transcribed pseudogene. With the knowledge that PIN1 specifically isomerizes phosphorylation of a serine or threonine that precedes proline and regulates the function of mitotic phosphoproteins, and hypothesizing that restoring the function of phosphorylated tau might prevent or reverse paired helical filament (PHF) formation in Alzheimer disease, Lu et al. (1999) demonstrated that the WW domain of PIN1 binds to phosphorylated tau at threonine-231 (,23I). The T231 residue is hyperphosphorylated in Alzheimer disease and is phosphorylated to a certain extent in the normal brain. Using a pulldown assay, Lu et al. (1999) demonstrated that PIN1 binds to hyperphosphorylated tau from the brains of people with Alzheimer disease but not to tau from age-matched healthy brains. By immunoblotting, Lu et al. (1999) detected endogenous PIN1 in the PHFs of diseased brains, and using immunohistochemistry, they found that recombinant PIN1 binds to pathologic tau. Using immunohistochemistry, Lu et al. (1999) localized PIN1 to the nucleus in healthy brains. In the brains of people with Alzheimer disease, PIN1 staining was associated with pathologic tau in neuronal cells. Lu et al. (1999) also demonstrated that phosphorylated tau could neither bind microtubules nor promote microtubule assembly. However, PIN1 was able to restore the ability of phosphorylated tau to bind microtubules and promoted microtubule assembly in vitro. The level of soluble PIN1 in the brains of Alzheimer patients was greatly reduced compared to that in age-matched control brains. The authors concluded with the hypothesis that since depletion of PIN1 induces mitotic arrest and apoptotic cell death, sequestration of PIN1 into PHFs may contribute to neuronal death. In the frog, Pin1 is implicated in the regulation of cell cycle progression and required for the DNA replication checkpoint. By fluorescence microscopy, Winkler et al. (2000) observed that nuclear extracts from Xenopus eggs depleted of Pin1 inappropriately transited from the G2 to the M phase of the cell cycle in the presence of a DNA replication inhibitor. Immunoblot analysis revealed that inappropriate transition was accompanied by hyperphosphorylation of CDC25 (see CDC25A), activation of CDC2 cyclin B and mitotic phosphoproteins. Addition of recombinant wildtype, but not mutant, Pin1 reversed the defect in replication checkpoint function.

The disclosed NOV34 nucleic acid of the invention encoding a Cyclophilin-type peptidyl-prolyl cis-trans isomerase-like protein includes the nucleic acid whose sequence is provided in Table 34A or a fragment thereof. The invention also includes a mutant or variant nucleic acid any of whose bases may be changed from the corresponding base shown in Table 34A while still encoding a protein that maintains its Cyclophilin-type peptidyl-prolyl cis-trans isomerase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37 percent of the bases may be so changed.

The disclosed NOV34 protein of the invention includes the Cyclophilin-type peptidyl-prolyl cis-trans isomerase-like protein whose sequence is provided in Table 34B. The invention also includes a mutant or variant protein any of whose residues may be changed from the corresponding residue shown in Table 34B while still encoding a protein that maintains its Cyclophilin-type peptidyl-prolyl cis-trans isomerase-like activities and physiological functions, or a functional fragment thereof. In the mutant or variant protein, up to about 24 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Cyclophilin-type peptidyl-prolyl cis-trans isomerase-like protein (NOV34) may function as a member of a “Cyclophilin-type peptidyl-prolyl cis-trans isomerase family”. Therefore, the NOV34 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV34 nucleic acids and proteins of the invention are useful in potential therapeutic applications implicated in diseases including but not limited to various pathologies and disorders as indicated below. For example, a cDNA encoding the Cyclophilin-type peptidyl-prolyl cis-trans isomerase-like protein (NOV34) may be useful in gene therapy, and the Cyclophilin-type peptidyl-prolyl cis-trans isomerase-like protein (NOV34) may be useful when administered to a subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Adrenoleukodystrophy, Congenital Adrenal Hyperplasia, Hemophilia, Hypercoagulation, Idiopathic thrombocytopenic purpura, Immunodeficiencies, Graft vesus host, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, or other pathologies or conditions. The NOV34 nucleic acid encoding the Cyclophilin-type peptidyl-prolyl cis-trans isomerase-like protein of the invention, or fragments thereof, may further be useful in diagnostic applications, wherein the presence or amount of the nucleic acid or the protein are to be assessed.

NOV34 nucleic acids and polypeptides are further useful in the generation of antibodies that bind immuno-specifically to the novel NOV34 substances for use in therapeutic or diagnostic methods. These antibodies may be generated according to methods known in the art, using prediction from hydrophobicity charts, as described in the “Anti-NOVX Antibodies” section below. The disclosed NOV34 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV35

A disclosed NOV35 nucleic acid of 1647 nucleotides (also referred to as CG57670-01) encoding a pyruvate kinase-like protein is shown in Table 35A. The start and stop codons are in bold letters.

TABLE 35A
NOV35 nucleotide sequence.

(SEQ ID NO:79)

GACCTCAGAAGCCATGTTGAAGCCCCATAGTGAAGCCAGGGCTGCCTTCATTCAGACCCAGCAGCTGCAC
GCAGCCATGGCTGACACATTCTTGGAGCACATGTGCTGCCTGGACACTGACTCGCCACCCATCACAGCCT
CGAGCACTGGCATCATCTGTACTATCGGCCCAGCTTCTCCATTGCTAGAGATGCTGAAGAAAACGATTAA
GTCTGGAATTAATGTGGCTCATCTCAACTCTCATGGAGCCCATGAGTACCATACAGAGACCATCAAGAAC
GTGCGCACAGCCACGGAAAGCTTTGCTTCTGACTCCATCCTCTACCAGCCCATTGCTGTGGCTCCAGACA
CTAAAGGACCTGAGATCCCAACTGGGCCCGTCAAGGGCAGCGCCACTGCAGACGTCGAGCTGAAGAAGGC
AGCCACTCTCAAGTTCACGCTGGATAATACCTACATGGAAAAGGGTAAAGAGAACATCCTGTGGCCCCAC
TACAAGAACATCTGCAAGGTGGTGGAAGTGGGCTGCAAGATCTACGTGGATGATGGGCTAATTTCTCTCC
AAGTGAAGCAGAAGGATGCTCACTTTCTGGTGACAGAGGTGGAAAATGGTGGCTCCTTGGGCAGCAAGAA
GAGTGTGAACCTTCCTGGGGCTGCCGTGGACCTGTCTGCCATGTTGGAGAAGGACATCCAGGACCTGAAG
TTTGGGGGCGAGCAAGATGTCGATATGATGTTTTCATCATTCATCTGCAAGACATCTGATGTCCATGAAG
TTAGGAAGGTCTTGGGAGAGAAAGGAAAGAACAGCAAGATAACCAGCAAAATTGAGAATCATGATCCGCG
TTGGAGGTTTGATGAAATCCTGGAGGCCAGCGATGGGATTATGGTAGCTCGTGGTGATCCACCACAAGCC
GTCGACATGGAGATTCCTGCAGGGAAGGTCTGCCTTGCTCAGAGGATGATGATTGGGTGGTGCAACCAAG
CTGGGAAGCCTGTCATCTTTGCCACTCAGATGCTAGAGGATGTGATCAAGAAGCTCCACCCCACTTGGGC
TGAGGGCAGTGGTGTCGCCAATGCAGTTCTGGTGGAAGCTGACTCCATCATGCTGTCTGGAGAAACAGCC
AAAGGGAACTATCCTCTGGAGGCTGTGCACATGCAGCACCTGATTGCCTGTGAGGCAGACCCCACCATCT
ACCACTTGCAATTATTTGAGGAGTTCTGCCACCTGGCACCCATTACCAGTGACCCCGCAGAAGCTACTGC
CATGGGCACTGTGGAGGCCTCCTTCAAGTGCTGCAGTGGGGCCATAATCGTCCTCACCAAGTCTGCCAGG
TGTGCCCACCAGGTCGCCACATACTGCCCACGTGCCCCCATGATTGTTGTGACATGGCATCCCCAGGCAG
CTCGCCAGGCCCACCTGTACCGTGGTATCTTCCCTGTGCTGTGTAACGACCCCATCCAGGAGCCCCACGC
TGAGGATGTGGACCTCCGAGTGAACTTGGCCATGAATGTTGGTAAGGCCCGAGGCTTCTTCAAGAAGCAT
GATGTGGTCATTGTGCTGACCTGGGGACACCCTGGCCCTGGCTTCTCCACCACCCTGTGTGTTATTCCTG
TGCTGTGATGGACTCCAGAGCTCTTCCTCCAGCCCCT

In a search of public sequence databases, the NOV35 nucleic acid sequence, located on chromsome 6 has 751 of 894 bases (84%) identical to a gb:GENBANK-ID:MMM2PK|acc:X97047.1 mRNA from Mus musculus (M.musculus mRNA for M2-type pyruvate kinase). Public nucleotide databases include all GenBank databases and the GeneSeq patent database.

The disclosed NOV35 polypeptide (SEQ ID NO:80) encoded by SEQ ID NO:79 has 534 amino acid residues and is presented in Table 35B using the one-letter amino acid code. Signal P, Psort and/or Hydropathy results predict that NOV35 has a signal peptide and is likely to be localized extracellularly with a certainty of 0.4500.

TABLE 35B
Encoded NOV35 protein sequence.

| | |
|---|---|
| MLKPHSEARAAFIQTQQLHAAMADTFLEHMCCLDTDSPPITAWSTGIICTMGPASPLLEM | (SEQ ID NO:78) |
| LKKTIKSGINVAHLNSHGAHEYHTETIKNVRTATESFASDSILYQPIAVAPDTKGPEIPT | |
| GPVKGSGTAEVELKKGATLKFTLDNTYMEKGKENILWRDYKNICKVVEVGCKIYVDDGLI | |
| SLQVKQKDAHFLVTEVENGGSLGSKKSVNLPGAAVDLSAMLEKDIQDLKFGGEQDVDMMF | |
| SSFICKTSDVHEVRKVLGEKGKNSKITSKIENHDGGWRFDEILEASDGIMVARGDPPQAV | |
| EMEIPAGKVCLAQRMMIGWCNQAGKPVIFATQMLEDVIKKLHPTWAEGSGVANAVLVEAD | |
| CIMLSGETAKGNYPLEAVHMQHLIACEAEATIYHLQLFEEFCHLAPITSDPAEATANGTV | |
| EASFKCCSGAIIVLTKSARCAHQVARYCPRAPMIVVTWHPQAARQAHLYRGIFPVLCKDP | |
| IQEPQAEDVDLRVNLAMNVGKARGFFKKDDVVIVLTWGHPGPCFSTTLCVIPVL | |

A search of sequence databases reveals that the NOV35 amino acid sequence has 433/533 (81%) identity and 458/533 (85%) similarity with pir-id:S30038 pyruvate kinase (EC 2.7.1.40), muscle splice form M2-human. Public amino acid databases include the GenBank databases, SwissProt, PDB and PIR.

NOV35 is expressed in at least Adrenal gland, Aorta, B-cells, Blood, Bone, Brain, Breast, CNS, Colon, Ear, Esophagus, Eye, Gall bladder, Germ Cell, Head and neck, heart, Kidney, Larynx, Liver, Lung, Lymph, Marrow, Muscle, Neural, Omentum, Ovary, Pancreas, Parathyroid, Peripheral nervous system, Placenta, Pooled, Prostate, Skin, Small intestine, Spleen, Stomach, Synovial membrane, Testis, Tissue culture, Tonsil, Uterus, Whole embryo, and adrenal gland. This information was derived by determining the tissue sources of the sequences that were included in the invention including but not limited to SeqCalling sources, Public EST sources, Literature sources, and/or RACE sources.

The disclosed NOV35 polypeptide has homology to the amino acid sequences shown in the BLASTP data listed in Table 35C.

TABLE 35C
BLAST results for NOV35

| Gene Index/ | | Length | Identity | Positives | |
|---|---|---|---|---|---|
| Identifier | Protein/Organism | (aa) | (%) | (%) | Expect |
| gi\|14750405\|ref\|XP_ | similar to | 531 | 433/534 | 458/534 | 0.0 |
| 037768.1\| | pyruvate kinase, | | (81%) | (85%) | |
| (XM_037768) | muscle (H. | | | | |
| | sapiens) | | | | |
| gi\|4505839\|ref\|NP_002645.1\| | pyruvate kinase, | 531 | 432/534 | 458/534 | 0.0 |
| (NM_002654) | muscle; Pyruvate | | (80%) | (84%) | |
| | kinase-3; Thyroid | | | | |
| | hormone-binding | | | | |
| | protein, | | | | |
| | cytosolic (p58) | | | | |
| | [Homo sapiens] | | | | |
| gi\|107554\|pir\|\|A33983 | pyruvate kinase | 531 | 431/534 | 457/534 | 0.0 |
| | (EC 2.7.1.40) | | (80%) | (84%) | |
| | isozyme M2 - | | | | |
| | human | | | | |
| gi\|266427\|sp\|P14618\| | Pyruvate kinase, | 531 | 433/534 | 458/534 | 0.0 |
| KPY1_HUMAN | M1 isozyme | | (81%) | (85%) | |
| | (Pyruvate kinase | | | | |
| | muscle isozyme) | | | | |
| | (Cytosolic | | | | |
| | thyroid hormone- | | | | |
| | binding protein) | | | | |
| | (CTHBP) (THBP1) | | | | |
| gi\|15215430\|gb\|AAH12811.1\| | (protein for | 531 | 431/534 | 457/534 | 0.0 |
| AAH12811 | MGC: 3932) [Homo | | (80%) | (84%) | |
| (BC012811) | sapiens] | | | | |

Tables 35D-E list the domain descriptions from DOMAIN analysis results against NOV35. This indicates that the NOV35 sequence has properties similar to those of other proteins known to contain this domain.

TABLE 35D
Domain Analysis of NOV35

gnl|Pfam|pfam02887, PK_C, Pyruvate kinase, alpha/beta domain
CD-Length = 116 residues, 99.1% aligned
Score = 129 bits (324), Expect = 4e−31

TABLE 35E
Domain Analysis of NOV35

gnl|Pfam|pfam00224, PK, Pyruvate kinase, barrel domain. This domain of
the is actually a small beta-barrel domain nested within a larger TIM
barrel. The active site is found in a cleft between the two domains.
CD-Length = 349 residues, 100.0% aligned
Score = 422 bits (1084), Expect = 3e−119

Pyruvate kinase is also known as ATP:pyruvate phosphotransferase (EC 2.7.1.40). At least 3 molecular forms with pyruvate kinase activity are known (Bigley et al., 1968). The form that is deficient in a type of hemolytic anemia is the red cell variety, PK1. PK2 is found in kidney. PK3 is found in leukocytes, muscle, platelets, and brain but not in red cells or kidney. PK1 is found also in liver. A patient with red cell PK deficiency has been found to have abnormal liver enzyme also (Bunn, 1981); see Nakashima et al. (1977). During fetal development, PK3 changes to PK1 in the liver. PK1 is a tetramer composed of two dissimilar polypeptides of somewhat different molecular weight. It is an allosteric enzyme exhibiting cooperative binding for phosphoenolpyruvate and sensitivity to fructose-1,6-diphosphate. PK3 also is a tetrameric protein but, unlike PK1, all subunits are alike and, not unexpectedly, there is no cooperative behavior. The enzyme is insensitive to fructose-1,6-diphosphate. Patients with deficiency of red cell PK have normal PK2 and PK3. Tsutsumi et al. (1988) showed that pyruvate kinase occurs in 4 isozymic forms (L, R, M1, M2) and that these are encoded by 2 different genes, PKL and PKM. The L and R isozymes are generated from the PKL gene by differential splicing of RNA; the M1 and M2 forms are produced from the PKM gene by differential splicing. Studies of somatic cell hybrids showed that the PK3 and MPI loci are syntenic (Shows, 1972). By cell hybridization studies, Van Heyningen et al. (1975) found that the MPI and PK3 loci are on chromosome 15. Chern et al. (1977) narrowed the assignment to 15q22-qter. Tani et al. (1988) isolated and sequenced 2 overlapping clones covering the entire coding sequence of PKM2. By in situ hybridization they demonstrated that the gene is located at band 15q22. Northern blot analysis with RNA from a human hepatoma demonstrated that the M2-type PK was predominantly expressed in hepatoma cells, whereas L-type PK was preferentially expressed in the nontumor portion of the liver. Takenaka et al. (1991) reported that the gene that encodes both the M1 and the M2 isozymes is approximately 32 kb long and comprises 12 exons and 11 introns. Exons 9 and 10 contain sequences specific for the M1 and M2 types, respectively, indicating that the human fetal and adult isozymes are produced from the same gene by alternative splicing.

The disclosed NOV35 nucleic acid of the invention encoding a pyruvate kinase-like protein includes the nucleic acid whose sequence is provided in Table 35A or a fragment thereof. The invention also includes a mutant or variant nucleic acid any of whose bases may be changed from the corresponding base shown in Table 35A while still encoding a protein that maintains its pyruvate kinase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 16 percent of the bases may be so changed.

The disclosed NOV35 protein of the invention includes the pyruvate kinase-like protein whose sequence is provided in Table 35B. The invention also includes a mutant or variant protein any of whose residues may be changed from the corresponding residue shown in Table 35B while still encoding a protein that maintains its pyruvate kinase-like activities and physiological functions, or a functional fragment thereof. In the mutant or variant protein, up to about 19 percent of the residues may be so changed.

The invention further encompasses antibodies and antibody fragments, such as F_{ab} or (F_{ab})₂ that bind immunospecifically to any of the proteins of the invention.

The above defined information for this invention suggests that this pyruvate kinase-like protein (NOV35) may function as a member of a “pyruvate kinase family”. Therefore, the NOV35 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV35 nucleic acids and proteins of the invention are useful in potential therapeutic applications implicated in diseases including but not limited to various pathologies and disorders as indicated below. For example, a cDNA encoding the pyruvate kinase-like protein (NOV35) may be useful in gene therapy, and the pyruvate kinase-like protein (NOV35) may be useful when administered to a subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Adrenoleukodystrophy, Congenital Adrenal Hyperplasia, Hemophilia, Hypercoagulation, Idiopathic thrombocytopenic purpura, Immunodeficiencies, Graft vesus host, Von Hippel-Lindau (VHL) syndrome, Alzheimer-'s disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, or other pathologies or conditions. The NOV nucleic acid encoding the pyruvate kinase-like protein of the invention, or fragments thereof, may further be useful in diagnostic applications, wherein the presence or amount of the nucleic acid or the protein are to be assessed.

NOV35 nucleic acids and polypeptides are further useful in the generation of antibodies that bind immuno-specifically to the novel NOV35 substances for use in therapeutic or diagnostic methods. These antibodies may be generated according to methods known in the art, using prediction from hydrophobicity charts, as described in the “Anti-NOVX Antibodies” section below. The disclosed NOV35 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV36

NOV36 includes two Cis/Trans Peptidyl Prolyl Isomerase-like proteins disclosed below. The disclosed sequences have been named NOV36a and NOV36b.

NOV36a

A disclosed NOV36a nucleic acid of 600 nucleotides (also referred to as CG57149-01) encoding a Cis/Trans Peptidyl Prolyl Isomerase-like protein is shown in Table 36A. The start and stop codons are in bold letters.

TABLE 36A
NOV36a nucleotide sequence.

(SEQ ID NO:81)

ACCAGGAGCCCTGTACTACCAGCCATGGTCAACCCCACCATGTTCTTCAACATCGCCATCAACAGCGAGG
CCTTGGGGCACGTCTCCTTCGAACTGTTTGCAGACAAGTTTCCAAAGACAGAAAACTTTCGTGCTCTGAG
CACTGGAGAGAAAGGATTTGGTTATAAGGGTTCCTGCTTTCACAGAATTATTCTAGGGCTTTTGTGTCAG
GGTGGTGACTTTACATGCCATAATGGCACTGGTGGCAAGTCTGTCTACAGGGACAAATTTGATGATGAGA
ACTTCATTCTGAAGCATACAGGTCCTGGCATCTTGTCCATGAAGCATACAGGTCCTGGCATCTTGTCCAT
GGCAAATGCTGGACCCAACACAAACGATTCCCAGATTTTCATCTGCACTGCCAAGACCGAGTGGTTGGAT
GGCAAGCATGTGGTCTCTGGCAGGGTGAAAGAAGGCATCAAGATTGTGGAGGCCATGAAGCGCTATGGGT
CCAAGAATGGCAAGAGCAGGAAGAAGATCACCACTGCTGACTGTGGACAACTCTAATAAGTTTGACTTGT
GTTTTATCTTAACCACCAGACCATTCCTTTTGTAGCTCAG

In a search of public sequence databases, the NOV36a nucleic acid sequence, located on chromsome 10 has 288 of 327 bases (88%) identical to a gb:GENBANK-ID:HSCYCR|acc:Y00052.1 mRNA from Homo sapiens (Human mRNA for T-cell cyclophilin). Public nucleotide databases include all GenBank databases and the GeneSeq patent database.

The disclosed NOV36a polypeptide (SEQ ID NO:82) encoded by SEQ ID NO:81 has 173 amino acid residues and is presented in Table 36B using the one-letter amino acid code. Signal P, Psort and/or Hydropathy results predict that NOV36a has no signal peptide and is likely to be localized at the plasma membrane with a certainty of 0.6000.

TABLE 36B
Encoded NOV36a protein sequence.

| | |
|---|---|
| MVNPTMFFNIAINSEALGHVSFELFADKFPKTENFRALSTGEKGFGYKGSCFHRIILGLL | (SEQ ID NO:82) |
| CQGGDFTCHNGTGGKSVYREKFDDENFILKHTGPGILSMKHTGPGILSMANAGPNTNDSQ | |
| IFICTAKTEWLDGKHVVSGRVKEGIKIVEAMKRYGSKNGKSRKKITTADCGQL | |

A search of sequence databases reveals that the NOV36a amino acid sequence has 136 of 173 amino acid residues (78%) identical to, and 149 of 173 amino acid residues (86%) similar to, the 165 amino acid residue ptnr:pir-id:CSHUA protein from human (peptidylprolyl isomerase (EC 5.2.1.8) A). Public amino acid databases include the GenBank databases, SwissProt, PDB and PIR.

NOV36b

A disclosed NOV36b nucleic acid of 566 nucleotides (also referred to as CG57149-02) encoding a Cis/Trans Peptidyl Prolyl Isomerase-like protein is shown in Table 36A. The start and stop codons are in bold letters.

TABLE 36C
NOV36b nucleotide sequence.

| | |
|---|---|
| GTACTACCAGCCATGGTCAACCCCACCATGTTCTTCAACATCGCCATCAACAGCGAGGCC | (SEQ ID NO:83) |
| TTGGGGCACGTCTCCTTCGAACTGTTTGCAGACAAGTTTCCAAAGACAGAAAACTTTCGT | |
| GCTCTGAGCACTGGAGAGAAAGGATTTGGTTATAAGGGTTCCTGCTTTCACAGAATTATT | |
| CTAGGGCTTTTGTGTCAGGGTGGTGACTTTACATGCCATAATGGCACTGGTGGCAAGTCT | |
| GTCTACAGGGAGAAATTTGATGATGAGAACTTCATTCTGAAGCATACAGGTCCTGGCATC | |
| TTGTCCATGAAGCATACAGGTCCTGGCATCTTGTCCATGGCAAATGCTGGACCCAACACA | |
| AACGATTCCCAGATTTTCATCTGCACTGCCAAGACCGAGTGGTTGGATGGCAAGCATGTG | |
| GTCTCTGGCAGGGTGAAAGAAGGCATCAAGATTGTGGAGGCCATGAAGCGCTATGGGTCC | |
| AAGAATGGCAAGAGCAGGAAGAAGATCACCACTGCTGACTGTGGACAACTCTAATAAGTT | |
| TGACTTGTGTTTTATCTTAACCACCA | |

In a search of public sequence databases, the NOV36b nucleic acid sequence has 269 of 311 bases (86%) identical to a gb:GENBANK-ID:AF139893|acc:AF139893.1 mRNA from Oryctolagus cuniculus (Oryctolagus cuniculus cyclophilin 18 mRNA, complete cds). Public nucleotide databases include all GenBank databases and the GeneSeq patent database.

The disclosed NOV36b polypeptide (SEQ ID NO:84) encoded by SEQ ID NO:83 has 173 amino acid residues and is presented in Table 36B using the one-letter amino acid code. Signal P, Psort and/or Hydropathy results predict that NOV36b has no signal peptide and is likely to be localized in the cytoplasm with a certainty of 0.4500.

TABLE 36D
Encoded NOV36b protein sequence.

| | |
|---|---|
| MVNPTMFFNIAINSEALGHVSFELFADKFPKTENFRALSTGEKGFGYKGSCFHRIILGLL | (SEQ ID NO:84) |
| CQGGDFTCHNGTGGKSVYREKFDDENFILKHTGPGILSMKHTGPGILSMANAGPNTNDSQ | |
| IFICTAKTEWLDGKHVVSGRVKEGIKIVEAMKRYGSKNGKSRKKITTADCGQL | |

A search of sequence databases reveals that the NOV36b amino acid sequence has 136 of 173 amino acid residues (78%) identical to, and 149 of 173 amino acid residues (86%) similar to, the 165 amino acid residue ptnr:TREMBLNEW-ACC:AA1100689 protein from Homo sapiens (Human) (PEPTIDYLPROLYL ISOMERASE A (CYCLOPHILIN A)). Public amino acid databases include the GenBank databases, SwissProt, PDB and PIR.

NOV36b is expressed in at least Epidermis, Lymphoid tissue. This information was derived by determining the tissue sources of the sequences that were included in the invention including but not limited to SeqCalling sources, Public EST sources, Literature sources, and/or RACE sources.

The disclosed NOV36a polypeptide has homology to the amino acid sequences shown in the BLASTP data listed in Table 36E.

TABLE 36E
BLAST results for NOV36a

| Gene Index/ | | Length | Identity | Positives | |
|---|---|---|---|---|---|
| Identifier | Protein/Organism | (aa) | (%) | (%) | Expect |
| gi\|12804335\|gb\|AAH03026.1\| | (protein for | 174 | 136/174 | 149/174 | 2e−70 |
| AAH03026 | IMAGE: 2823490) | | (78%) | (85%) | |
| (BC003026) | [Homo sapiens] | | | | |
| gi\|4033689\|sp\|P04374\| | PEPTIDYL-PROLYL | 164 | 136/174 | 149/174 | 8e−70 |
| CYPH_BOVIN | CIS-TRANS | | (78%) | (85%) | |
| | ISOMERASE A | | | | |
| | (PPIASE) | | | | |
| | (ROTAMASE) | | | | |
| | (CYCLOPHILIN A) | | | | |
| | (CYCLOSPORIN A- | | | | |
| | BINDING PROTEIN) | | | | |
| gi\|10863927\|ref\|NP_ | peptidylprolyl | 165 | 136/174 | 149/174 | 1e−69 |
| 066953.1\| | isomerase A | | (78%) | (85%) | |
| (NM_021130 | (cyclophilin A) | | | | |
| | [Homo sapiens] | | | | |
| gi\|68401\|pir\|\|CSBOAB | peptidylprolyl | 163 | 135/173 | 148/173 | 4e−69 |
| | isomerase (EC | | (78%) | (85%) | |
| | 5.2.1.8) A - | | | | |
| | bovine | | | | |
| gi\|13543666\|gb\|AAH05982.1\| | peptidylprolyl | 165 | 136/174 | 149/174 | 6e−69 |
| AAH05982 | isomerase A | | (78%) | (85%) | |
| (BC005982) | (cyclophilin A) | | | | |
| | [Homo sapiens] | | | | |

Table 36F lists the domain descriptions from DOMAIN analysis results against NOV36a. This indicates that the NOV36a sequence has properties similar to those of other proteins known to contain this domain.

TABLE 36F
Domain Analysis of NOV36

gnl|Pfam|pfam00160, pro_isomerase, Cyclophilin type peptidyl-prolyl
cis-trans isomerase
CD-Length = 162 residues, 100.0% aligned
Score = 196 bits (497), Expect = 1e−51

The human parvulin Pin1 is a member of the peptidyl-prolyl cis-trans isomerase group of proteins, which modulate the assembly, folding, activity, and transport of essential cellular proteins. Pin1 is a mitotic regulator interacting with a range of proteins that are phosphorylated before cell division. In addition, an involvement of Pin1 in the tau-related neurodegenerative brain disorders has recently been shown. In this context, Pin1 becomes depleted from the nucleus in Alzheimer's disease (AD) neurons when it is redirected to the large amounts of hyperphosphorylated tau associated with the neurofibrillary tangles. This depletion from the nucleus may ultimately contribute to neuron cell death. The 131-amino acid residue parvulin-like human peptidyl-prolyl cis/trans isomerase (PPIase) hPar14 was shown to exhibit sequence similarity to the regulator enzyme for cell cycle transitions human hPin1, but specificity for catalyzing pSer(Thr)-Pro cis/trans isomerizations was lacking. That FK and CsA completely inhibit immune function without completely inhibiting CN suggests that the inhibition of immune function is not mediated by general CN inhibition but by inhibition of a subset of CN which is critical for lymphocyte activation.

The disclosed NOV36b nucleic acid of the invention encoding a Cis/Trans Peptidyl Prolyl Isomerase-like protein includes the nucleic acid whose sequence is provided in Table 36A or a fragment thereof. The invention also includes a mutant or variant nucleic acid any of whose bases may be changed from the corresponding base shown in Table 36A while still encoding a protein that maintains its Cis/Trans Peptidyl Prolyl Isomerase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 14 percent of the bases may be so changed.

The disclosed NOV36b protein of the invention includes the Cis/Trans Peptidyl Prolyl Isomerase-like protein whose sequence is provided in Table 36B. The invention also includes a mutant or variant protein any of whose residues may be changed from the corresponding residue shown in Table 36B while still encoding a protein that maintains its Cis/Trans Peptidyl Prolyl Isomerase-like activities and physiological functions, or a functional fragment thereof. In the mutant or variant protein, up to about 22 percent of the residues may be so changed.

The invention further encompasses antibodies and antibody fragments, such as F_{ab}, or (F_{ab})₂, that bind immunospecifically to any of the proteins of the invention.

The above defined information for this invention suggests that this Cis/Trans Peptidyl Prolyl Isomerase-like protein (NOV36) may function as a member of a “Cis/Trans Peptidyl Prolyl Isomerase family”. Therefore, the NOV36 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV36 nucleic acids and proteins of the invention are useful in potential therapeutic applications implicated in diseases including but not limited to various pathologies and disorders as indicated below. For example, a cDNA encoding the Cis/Trans Peptidyl Prolyl Isomerase-like protein (NOV36) may be useful in gene therapy, and the Cis/Trans Peptidyl Prolyl Isomerase-like protein (NOV36) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV36 nucleic acid encoding the Cis/Trans Peptidyl Prolyl Isomerase-like protein of the invention, or fragments thereof, may further be useful in diagnostic applications, wherein the presence or amount of the nucleic acid or the protein are to be assessed.

NOV36 nucleic acids and polypeptides are further useful in the generation of antibodies that bind immuno-specifically to the novel NOV substances for use in therapeutic or diagnostic methods. These antibodies may be generated according to methods known in the art, using prediction from hydrophobicity charts, as described in the “Anti-NOVX Antibodies” section below. The disclosed NOV36 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV37

A disclosed NOV37 nucleic acid of 660 nucleotides (also referred to as CG57151-01) encoding a Cis/Trans Peptidyl Prolyl Isomerase-like protein is shown in Table 37A. The start and stop codons are in bold letters.

TABLE 37A
NOV37 nucleotide sequence.

(SEQ ID NO:85)

ACTAGTCATTCTTCCCAGTAGCTAATGAAGCTGACTTTTAAAAAGAAGGCTGTGAGCTTTGCAGATGCTG
CTGCCGCCCAGGGCCCCCTGCTTCCAGCCATGGTCAACCCCACCATGTTTTTCCACATTGCTGTCGATGG
CGAGCCCTTGGGCTGTGTCTCCTTCGAGGTAGAGCTGTTTGCAGACAAGGTTCCAAAGACAGCAGAAAAT
TTCCATGCTCTGAGCACTGGAGAAAAAGGATTTGGTTATAAGGGTTCCTGCTTTCACAGAATTATTCCAG
GGTTTACGTGTCAGAGTGGTGACTTCACACGCCATGGTGGCAAGTCCATCTGCAGGGAGAAATTTGATGA
CAAGAACTTCATCCTGAAGCATACGGGTCCTGGCATCTTGTCCATGGCAAATGCTGGACCCAGCGTGAAC
GTTTCCCAGTTTTTTATCTGCCCTGCCAAGACAGAGTGGTTGGATTGCAAGCATGTGGTCTTTGGCAAGG
TGAAAGATGGCATGAATATTGTGGAGGTCATGGAGCACTTGGGGTCCAAGAATGGCAAGATCAGCAAGAA
GATCACCATTGCTGACTGGACAACTGCAATAAATTTGACGGGTGTTTCTCTTAAAAAAAAAAAAAAAATA
CTGTGACAGACCAAGGTAAATTGTTTTTGA

In a search of public sequence databases, the NOV37 nucleic acid sequence, located on chromsome 10 has 492 of 581 bases (85%) identical to a (HSCPH192|acc: X52857.1) cyclophilin-related processed pseudogene mRNA from human. Public nucleotide databases include all GenBank databases and the GeneSeq patent database.

The disclosed NOV37 polypeptide (SEQ ID NO:86) encoded by SEQ ID NO:85 has 203 amino acid residues and is presented in Table 37B using the one-letter amino acid code. Signal P, Psort and/or Hydropathy results predict that NOV37 has no signal peptide and is likely to be localized in the cytoplasm with a certainly of 0.6400.

TABLE 37B
Encoded NOV37 protein sequence.

| | |
|---|---|
| MKLTFKKKAVSFADAAAAQGPLLPAMVNPTMFFHIAVDGEPLGCVSFEVELFADKVPKTA | (SEQ ID NO:86) |
| ENFHALSTGEKGFGYKGSCFHRIIPGFTCQSGDFTRHGGKSICREKFDDKNFILKHTGPG | |
| ILSMANAGPSVNVSQFFICPAKTEWLDCKHVVFGKVKDGMNIVEVMEHLGSKNGKISKKi | |
| TIADWTTAINLTGVSLKKKKKIL | |

A search of sequence databases reveals that the NOV37 amino acid sequence has 136 of 160 amino acid residues (85%) identical to, and 142 of 160 amino acid residues (88%) similar to, the 165 amino acid residue ptnr:pir-id:CSHUA protein from human (peptidylprolyl isomerase (EC 5.2.1.8) A). Public amino acid databases include the GenBank databases, SwissProt, PDB and PIR.

NOV37 is expressed in at least Bone Marrow, Brain, Cartilage, Cochlea, Colon, Epidermis, Kidney, Lung, Mammary gland/Breast, Ovary, Pancreas, Prostate, Stomach, Testis, Thymus, Umbilical Vein, Uterus, Vulva, Whole Organism. Expression information was derived from the tissue sources of the sequences that were included in the derivation of the sequence of CG57151_{—}01. The sequence is predicted to be expressed in the following tissues because of the expression pattern of (GENBANK-ID: pir-id:CSHUA peptidylprolyl isomerase) a closely related peptidylprolyl isomerase homolog in species human: Kidney, Lung. This information was derived by determining the tissue sources of the sequences that were included in the invention including but not limited to SeqCalling sources, Public EST sources, Literature sources, and/or RACE sources.

The disclosed NOV37 polypeptide has homology to the amino acid sequences shown in the BLASTP data listed in Table 37C.

TABLE 37C
BLAST results for NOV37

| Gene Index/ | | Length | Identity | Positives | |
|---|---|---|---|---|---|
| Identifier | Protein/Organism | (aa) | (%) | (%) | Expect |
| gi\|12804335\|gb\|AAH03026.1\| | (protein for | 174 | 139/171 | 147/171 | E3−71 |
| AAH03026 | IMAGE: 2823490) [Homo | | (81%) | (85%) | |
| (BC003026) | sapiens] | | | | |
| gi\|4033689\|sp\|P04374\| | PEPTIDYL-PROLYL | 164 | 137/162 | 142/162 | 4e−70 |
| CYPH_BOVIN | CIS-TRANS | | (84%) | (87%) | |
| | ISOMERASE A | | | | |
| | (PPIASE) | | | | |
| | (ROTAMASE) | | | | |
| | (CYCLOPHILIN A) | | | | |
| | (CYCLOSPORIN A- | | | | |
| | BINDING PROTEIN) | | | | |
| gi\|10863927\|ref\|NP_ | peptidylprolyl | 165 | 136/162 | 142/162 | 1e−69 |
| 066953.1\| | isomerase A | | (83%) | (86%) | |
| (NM_021130) | (cyclophilin A) [Homo | | | | |
| | sapiens] | | | | |
| gi\|68401\|pir\|\|CSBOAB | peptidylprolyl | 163 | 136/161 | 141/161 | 2e−69 |
| | isomerase (EC | | (84%) | (87%) | |
| | 5.2.1.8) A - | | | | |
| | bovine | | | | |
| gi\|13937981\|gb\|AAH07104.1\| | peptidylprolyl | 165 | 136/162 | 141/162 | 2e−69 |
| AAH07104 | isomerase A | | (83%) | (86%) | |
| (BC007104) | (cyclophilin A) | | | | |
| | [Homo sapiens] | | | | |

Table 37D lists the domain descriptions from DOMAIN analysis results against NOV37. This indicates that the NOV37 sequence has properties similar to those of other proteins known to contain this domain.

TABLE 37D
Domain Analysis of NOV37

gnl|Pfam|pfam00160, pro_isomerase, Cyclophilin type peptidyl-prolyl
cis-trans isomerase
CD-Length = 162 residues, 97.5% aligned
Score = 191 bits (484), Expect = 5e−50

The disclosed NOV37 nucleic acid of the invention encoding a Cis/Trans Peptidyl Prolyl Isomerase-like protein includes the nucleic acid whose sequence is provided in Table 37A or a fragment thereof. The invention also includes a mutant or variant nucleic acid any of whose bases may be changed from the corresponding base shown in Table 37A while still encoding a protein that maintains its Cis/Trans Peptidyl Prolyl Isomerase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 15 percent of the bases may be so changed.

The disclosed NOV37 protein of the invention includes the Cis/Trans Peptidyl Prolyl Isomerase-like protein whose sequence is provided in Table 37B. The invention also includes a mutant or variant protein any of whose residues may be changed from the corresponding residue shown in Table 37B while still encoding a protein that maintains its Cis/Trans Peptidyl Prolyl Isomerase-like activities and physiological functions, or a functional fragment thereof. In the mutant or variant protein, up to about 15 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Cis/Trans Peptidyl Prolyl Isomerase-like protein (NOV37) may function as a member of a “Cis/Trans Peptidyl Prolyl Isomerase family”. Therefore, the NOV37 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV37 nucleic acids and proteins of the invention are useful in potential therapeutic applications implicated in diseases including but not limited to various pathologies and disorders as indicated below. For example, a cDNA encoding the Cis/Trans Peptidyl Prolyl Isomerase-like protein (NOV37) may be useful in gene therapy, and the Cis/Trans Peptidyl Prolyl Isomerase-like protein (NOV37) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV37 nucleic acid encoding the Cis/Trans Peptidyl Prolyl Isomerase-like protein of the invention, or fragments thereof, may further be useful in diagnostic applications, wherein the presence or amount of the nucleic acid or the protein are to be assessed.

NOV37 nucleic acids and polypeptides are further useful in the generation of antibodies that bind immuno-specifically to the novel NOV37 substances for use in therapeutic or diagnostic methods. These antibodies may be generated according to methods known in the art, using prediction from hydrophobicity charts, as described in the “Anti-NOVX Antibodies” section below. The disclosed NOV37 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV38

A disclosed NOV38 nucleic acid of 600 nucleotides (also referred to as CG57153-01) encoding a Cis/Trans Peptidyl Prolyl Isomerase-like protein is shown in Table 38A. The start and stop codons are in bold letters.

TABLE 38A
NOV38 nucleotide sequence.

(SEQ ID NO:87)

AATTTATTGGTTTGTTTGTTTAAAATATTTGTTGGCACTTTGCAGATGCCACTTCCACTGATGTCACCAC
TGCCAGTGATGGTCACCTCCACCTTGTTCTTTAACTTTGTAGTCAACGGTGAGCACTTGGGCCATGTCTC
CTTCCAGCTGTTTGCAAAGAAAGTTCCAAAGACAGCAGAAAATGTTCATTTTGTGAGCACTGGAGAGAAA
GGATTTGGCTATAAGTGTTCCTGTTTTCACAGAATTATTCCAGGGTTTATATGCCAGAGTGGTGACTTCA
CATGTCATGATGACACTGGCACAAAGTCCAACTACTGGGAGAAGTCTGATCATGATAACTCCATCCTGAA
GCATACAAGACCTGGCACCTTGTCCATGGCAAATACTGGACGCTACACAAATGGTTTCCAGTTTTTCATC
TGCACTGCCAAAACTGTGTGGTTGGGTGGCAAGAGTGCAGTCTTTGGCAAGACAAAAGAGGGCTTGAATA
TCTTGGAAGCCATGGCGCACTTTGCTTTCTGGAATGGCAAAACCAGAAAGAAGACCACGATTGACAACTG
TGGACAACTCCAATAAATTTAACTTATGTTTTGTTTTAAC

In a search of public sequence databases, the NOV38 nucleic acid sequence, located on chromsome 16 has 456 of 564 bases (80%) identical to a gb:GENBANK-ID:AK026569|acc:AK026569.1 mRNA from Homo sapiens (Homo sapiens cDNA: FLJ22916 fis, clone KAT06406, highly similar to HSCYCR Human mRNA for T-cell cyclophilin. Public nucleotide databases include all GenBank databases and the GeneSeq patent database.

The disclosed NOV38 polypeptide (SEQ ID NO:88) encoded by SEQ ID NO:87 has 176 amino acid residues and is presented in Table 38B using the one-letter amino acid code. Signal P, Psort and/or Hydropathy results predict that NOV38 has no signal peptide and is likely to be localized in the cytoplasm with a certainty of 0.5500.

TABLE 38B
Encoded NOV38 protein sequence.

| | |
|---|---|
| MPLPLMSPLPVMVTSTLFFNFVVNGEHLGHVSFQLFAKKVPKTAENVHFVSTGEKGFGYK | (SEQ ID NO:88) |
| CSCFHRIIPGFICQSGDFTCHDDTGTKSNYWEKSDDDNSILKHTRPGTLSMANTGRYTNG | |
| FQFFICTAKTVWLGGKSAVFGKTKEGLNILEAMAHFAFWNGKTRKKTTIDNCGQLQ | |

A search of sequence databases reveals that the NOV38 amino acid sequence has 113 of 164 amino acid residues (68%) identical to, and 127 of 164 amino acid residues (77%) similar to, the 164 amino acid residue ptnr:SPTREMBL-ACC:Q9TTC6 protein from Oryctolagus cuniculus (Rabbit) (CYCLOPHILIN 18). Public amino acid databases include the GenBank databases, SwissProt, PDB and PIR.

NOV38 is expressed in at least Bone Marrow, Brain, Cartilage, Cochlea, Colon, Epidermis, Kidney, Lung, Mammary gland/Breast, Ovary, Pancreas, Prostate, Stomach, Testis, Thymus, Umbilical Vein, Uterus, Vulva, and Whole Organism. Expression information was derived from the tissue sources of the sequences that were included in the derivation of the sequence of CG57153_{—}01. The sequence is predicted to be expressed in the following tissues because of the expression pattern of (GENBANK-ID: gb:GEN13ANK-ID:AK026569|acc:AK026569.1) a closely related Homo sapiens cDNA: 1FLJ22916 fis, clone KAT06406, highly similar to HSCYCR Human mRNA for T-cell cyclophilin homolog in species Homo sapiens. This information was derived by determining the tissue sources of the sequences that were included in the invention including but not limited to SeqCalling sources, Public EST sources, Literature sources, and/or RACE sources.

The disclosed NOV38 polypeptide has homology to the amino acid sequences shown in the BLASTP data listed in Table 38C.

TABLE 38C
BLAST results for NOV38

| Gene Index/ | | Length | Identity | Positives | |
|---|---|---|---|---|---|
| Identifier | Protein/Organism | (aa) | (%) | (%) | Expect |
| gi\|12804335\|gb\|AAH03026.1\| | (protein for | 174 | 115/168 | 128/168 | 6e−58 |
| AAH03026 | IMAGE: 2823490) | | (68%) | (75%) | |
| (BC003026) | [Homo sapiens] | | | | |
| gi\|13937981\|gb\|AAH07104.1\| | peptidylprolyl | 165 | 114/165 | 127/165 | 2e−57 |
| AAH07104 | isomerase A | | (69%) | (76%) | |
| (BC007104) | (cyclophilin A) | | | | |
| | [Homo sapiens] | | | | |
| gi\|10863927\|ref\|NP_ | peptidylprolyl | 165 | 114/165 | 127/165 | 4e−57 |
| 066953.1\| | isomerase A | | (69%) | (76%) | |
| (NM_021130) | (cyclophilin A) | | | | |
| | [Homo sapiens] | | | | |
| gi\|4033689\|sp\|P04374\| | PEPTIDYL-PROLYL | 154 | 114/164 | 126/164 | 4e−57 |
| CYPH_BOVIN | CIS-TRANS | | (69%) | (76%) | |
| | ISOMERASE A | | | | |
| | (PPIASE) | | | | |
| | (ROTAMASE) | | | | |
| | (CYCLOPHILIN A) | | | | |
| | (CYCLOSPORIN A- | | | | |
| | BINDING PROTEIN) | | | | |
| gi\|6651171\|gb\|AAF22215.1\| | cyclophilin 18 | 154 | 113/164 | 127/164 | 7e−57 |
| AF139893_1 | [Oryctolagus | | (68%) | (76%) | |
| (AF139893) | cuniculus] | | | | |

Table 38D lists the domain descriptions from DOMAIN analysis results against NOV38. This indicates that the NOV38 sequence has properties similar to those of other proteins known to contain this domain.

TABLE 38D
Domain Analysis of NOV 38

gnl|Pfam|pfam00160, pro_isomerase, Cyclophilin type peptidyl-prolyl
cis-trans isomerase.
CD-Length = 162 residues, 100.0% aligned
Score = 171 bits (433), Expect = 3e−44

The disclosed NOV38 nucleic acid of the invention encoding a Cis/Trans Peptidyl Prolyl Isomerase-like protein includes the nucleic acid whose sequence is provided in Table 38A or a fragment thereof. The invention also includes a mutant or variant nucleic acid any of whose bases may be changed from the corresponding base shown in Table 38A while still encoding a protein that maintains its Cis/Trans Peptidyl Prolyl Isomerase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 20 percent of the bases may be so changed.

The disclosed NOV38 protein of the invention includes the Cis/Trans Peptidyl Prolyl Isomerase-like protein whose sequence is provided in Table 38B. The invention also includes a mutant or variant protein any of whose residues may be changed from the corresponding residue shown in Table 38B while still encoding a protein that maintains its Cis/Trans Peptidyl Prolyl Isomerase-like activities and physiological functions, or a functional fragment thereof. In the mutant or variant protein, up to about 32 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Cis/Trans Peptidyl Prolyl Isomerase-like protein (NOV38) may function as a member of a “Cis/Trans Peptidyl Prolyl Isomerase family”. Therefore, the NOV38 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV38 nucleic acids and proteins of the invention are useful in potential therapeutic applications implicated in diseases including but not limited to various pathologies and disorders as indicated below. For example, a cDNA encoding the Cis/Trans Peptidyl Prolyl Isomerase-like protein (NOV38) may be useful in gene therapy, and the Cis/Trans Peptidyl Prolyl Isomerase-like protein (NOV38) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV38 nucleic acid encoding the Cis/Trans Peptidyl Prolyl Isomerase-like protein of the invention, or fragments thereof, may further be useful in diagnostic applications, wherein the presence or amount of the nucleic acid or the protein are to be assessed.

NOV38 nucleic acids and polypeptides are further useful in the generation of antibodies that bind immuno-specifically to the novel NOV38 substances for use in therapeutic or diagnostic methods. These antibodies may be generated according to methods known in the art, using prediction from hydrophobicity charts, as described in the “Anti-NOVX Antibodies” section below. The disclosed NOV38 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV39

A disclosed NOV39 nucleic acid of 600 nucleotides (also referred to as CG57155-01) encoding a Cis/Trans Peptidyl Prolyl Isomerase-like protein is shown in Table 39A.

TABLE 39A
NOV39 nucleotide sequence.

(SEQ ID NO:89)

CTGAAAACTTTCTTGTAAGACTTTGACCTGCATTATATGATTCTCCTTAATCTTCACAGCATGATTTCGT
GTTTTTGGACATTTCTATACTGTGATGTGTGTCCCAAAACATGTAAAAATTTTCAGGTCTTGTGCACAGG
AAAAGCAGGGTTTTCTCAACGTGGCATAAGACTACATTACAAAAATTCCATTTTTCATCGAATAGTACAG
AATGGCTGGATACAAGGAGGGGATATAGTGTATGGAAAAGGAGATAATGGAGAGTCGATTTATGGTCCAA
CATTTGAAGATGAAAACTTTTCAGTTCCTCATAATAAAAGAGGAGTACTTGGAATGGCCAACAAAGGCCG
TCACAGCAACGGGTCACAATTCTATATCACACTGCAAGCAACTCCTTATCTAGATAGAAAATTTGTGGCT
TTTGGGTATGTATATTGTAGATCTATTTATATAATATTCACACCTGGTAGTAAAAAAGCCCAGAGAAGTA
TGTGCAAGAAACTAACAGTATGTGGTTGTGGGCGTAGTTTTTCAAAGGAAGAAGTAGTCAAATGCTGTAA
CAAGGACAACTCATCTTGAAACACTTACGCAGTGGTGTGT

In a search of public sequence databases, the NOV39 nucleic acid sequence, located on chromsome 6 has 257 of 397 bases (64%) identical to a gb:GENBANK-ID:AF043642|acc:AF043642.1 mRNA from Rattus norvegicus (Rattus norvegicus matrin cyclophilin (matrin-cyp) mRNA, complete cds). Public nucleotide databases include all GenBank databases and the GeneSeq patent database.

The disclosed NOV39 polypeptide (SEQ ID NO:90) encoded by SEQ ID NO:89 has 180 amino acid residues and is presented in Table 39B using the one-letter amino acid code. Signal P, Psort and/or Hydropathy results predict that NOV39 has a signal peptide and is likely to be localized extracellularly with a certainty of 0.5128. The most likely cleavage site for a NOV39 peptide is between amino acids 19 and 20.

TABLE 39B
Encoded NOV39 protein sequence.

(SEQ ID NO:90)

MILLNLHSMISCFWTFLYCDVCPKTCKNFQVLCTGKAGFSQRGIRLHYKNSIFHRIVQNGWIQGGDIVYG
KGDNGESIYGPTFEDENFSVPHNKRGVLGMANKGRHSNCSQFYITLQATPYLDRKFVAFGYVYCRSIYII
FTPGSKKAQRSMCKKLTVCGCGRSFSKEEVVKCCNKDNSS

A search of sequence databases reveals that the NOV39 amino acid sequence has 70 of 87 amino acid residues (80%) identical to, and 80 of 87 amino acid residues (91%) similar to, the 278 amino acid residue ptnr:TREMBLNEW-ACC:BAB29003 protein from Mus musculus (Mouse) (ADULT MALE HIPPOCAMPUS CDNA, RIKEN FULL-LENGTH ENRICHED LIBRARY, CLONE:2900084F20, FULL INSERT SEQUENCE). Public amino acid databases include the GenBank databases, SwissProt, PDB and PIR.

NOV39 is expressed in at least: Kidney, Lung, Lymphoid tissue, Mammary gland/Breast, Oviduct/Uterine Tube/Fallopian tube, Testis, Whole Organism. Expression information was derived from the tissue sources of the sequences that were included in the derivation of the sequence of CG57155_{—}01. The sequence is predicted to be expressed in the following tissues because of the expression pattern of (GENBANK-ID: gb:GENBANK-ID:AF043642|acc:AF043642.1) a closely related Rattus norvegicus matrin cyclophilin (matrin-cyp) mRNA, complete cds homolog in species Rattus norvegicus: Kidney, Lung, Lymphoid tissue. This information was derived by determining the tissue sources of the sequences that were included in the invention including but not limited to SeqCalling sources, Public EST sources, Literature sources, and/or RACE sources.

The disclosed NOV39 polypeptide has homology to the amino acid sequences shown in the BLASTP data listed in Table 39C.

TABLE 39C
BLAST results for NOV39

| Gene Index/ | | Length | Identity | Positives | |
|---|---|---|---|---|---|
| Identifier | Protein/Organism | (aa) | (%) | (%) | Expect |
| gi\|12851324\|dbj\|BAB29003.1\| | similar to NK- | 278 | 70/87 | 80/87 | 2e−41 |
| (AK013818) | TUMOR RECOGNITION | | (80%) | (91%) | |
| | PROTEIN (NATURAL- | | | | |
| | KILLER CELLS | | | | |
| | CYCLOPHILIN- | | | | |
| | RELATED PROTEIN) | | | | |
| | (NK-TR PROTEIN) | | | | |
| | [Mus musculus] | | | | |
| gi[6841028\|gb\|AAF28867.1\| | cyclophilin | 181 | 65/120 | 89/120 | 3e−35 |
| (AF121134) | [Schistosoma | | (54%) | (74%) | |
| | mansoni] | | | | |
| gi\|13929124\|ref\|NP_ | matrin | 752 | 64/117 | 85/117 | 1e−31 |
| 113981.1\| | cyclophilin | | (54%) | (71%) | |
| (NM_031793) | (matrin-cyp) | | | | |
| | [Rattus | | | | |
| | norvegicus] | | | | |
| gi\|6754858\|ref\|NP_035048.1\| | natural killer | 1482 | 63/117 | 82/117 | 2e−31 |
| (NM_010918) | tumor recognition | | (53%) | (69%) | |
| | [Mus musculus] | | | | |
| gi\|8039799\|sp\|P30415\| | NK-tumor recognition | 1453 | 63/117 | 82/117 | 2e−31 |
| NKCR_MOUSE | protein (Natural-killer | | (53%) | (69%) | |
| | cells; cyclophilin- | | | | |
| | related protein) (NK- | | | | |
| | TR protein) | | | | |

Table 39D lists the domain descriptions from DOMAIN analysis results against NOV39. This indicates that the NOV39 sequence has properties similar to those of other proteins known to contain this domain.

TABLE 39E
Domain Analysis of NOV39

gnl|Pfam|pfam00160, pro_isomerase, Cyclophilin type peptidyl-prolyl
cis-trans isomerase
CD-Length = 162 residues, 67.9% aligned
Score = 141 bits (356), Expect = 3e−35

The disclosed NOV39 nucleic acid of the invention encoding a Cis/Trans Peptidyl Prolyl Isomerase-like protein includes the nucleic acid whose sequence is provided in Table 39A or a fragment thereof. The invention also includes a mutant or variant nucleic acid any of whose bases may be changed from the corresponding base shown in Table 39A while still encoding a protein that maintains its Cis/Trans Peptidyl Prolyl Isomerase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, Up to about 36 percent of the bases may be so changed.

The disclosed NOV39 protein of the invention includes the Cis/Trans Peptidyl Prolyl Isomerase-like protein whose sequence is provided in Table 39B. The invention also includes a mutant or variant protein any of whose residues may be changed from the corresponding residue shown in Table 39B while still encoding a protein that maintains its Cis/Trans Peptidyl Prolyl Isomerase-like activities and physiological functions, or a functional fragment thereof. In the mutant or variant protein, up to about 20 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Cis/Trans Peptidyl Prolyl Isomerase-like protein (NOV39) may function as a member of a “Cis/Trans Peptidyl Prolyl Isomerase family”. Therefore, the NOV39 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV39 nucleic acids and proteins of the invention are useful in potential therapeutic applications implicated in diseases including but not limited to various pathologies and disorders as indicated below. For example, a cDNA encoding the Cis/Trans Peptidyl Prolyl Isomerase-like protein (NOV39) may be useful in gene therapy, and the Cis/Trans Peptidyl Prolyl Isomerase-like protein (NOV39) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV39 nucleic acid encoding the Cis/Trans Peptidyl Prolyl Isomerase-like protein of the invention, or fragments thereof, may further be useful in diagnostic applications, wherein the presence or amount of the nucleic acid or the protein are to be assessed.

NOV39 nucleic acids and polypeptides are further useful in the generation of antibodies that bind immuno-specifically to the novel NOV39 substances for use in therapeutic or diagnostic methods. These antibodies may be generated according to methods known in the art, using prediction from hydrophobicity charts, as described in the “Anti-NOVX Antibodies” section below. The disclosed NOV39 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV40

A disclosed NOV40 nucleic acid of 572 nucleotides (also referred to as CG57157-01) encoding a Cis/Trans Peptidyl Prolyl Isomerase-like protein is shown in Table 40A. The start and stop codons are in bold letters.

TABLE 40A
NOV40 nucleotide sequence (SEQ ID NO:91).

TTTGTAGTCATCGCTGCCACCTGAAGCCACCTGCCTCTAGCCATGGTCAA
CGCCCCACTGTGTTCTTTTGACATCATTGTTGATGGTAACTCCTTTGGCC
CATGCAGCTCCTTCGAGCTGTTTGCCGACAAAGTTCCAAAAACAGTGGAA
AACTTTCGTGCACTGAGCACTGGAGGAAAAGGATTTGCTTATAAGGGTTC
CTGCTTTCACAGAATTATTCCAGGGTTTATTTTATCTGCCAGAGTGCTGA
CTTCACACACCATAATAATGCCCCAGTCCATCTACCAGGAGAAATTTGAT
GATGAGAACTTCATCTTGAAGCACACAGGTCCTGGCATCTTGTCCATGGC
AAATGCTGGCCCGGACACAAATGGTTCCCAGTTTTTCACCTGTGTGGCCA
AGACTGAGTGGCTGGATGGCAAGCACAAGGTCTTTGGCAAAGTGAGAAGA
GGGGTGAATATCATGGAAGCCATGGAGTGCTCTGGGTCCGGGAATGGTGA
GACTGGCAAGAAGATCACCACTGCCAACTGCGGACAACTCTAATCAATCT
GCTTGTGTTTGATCTTAACCAC

In a search of public sequence databases, the NOV40 nucleic acid sequence, located on chromsome 17 has has 446 of 535 bases (83%) identical to a gb:GENBANK-ID:HSCYCR|acc:Y00052.1 mRNA from Homo sapiens (Human mRNA for T-cell cyclophilin). Public nucleotide databases include all GenBank databases and the GeneSeq patent database.

The disclosed NOV40 polypeptide (SEQ ID NO:92) encoded by SEQ ID NO:91 has 166 amino acid residues and is presented in Table 40B using the one-letter amino acid code. Signal P, Psort and/or Hydropathy results predict that NOV40 has no signal peptide and is likely to be localized at the plasma membrane with a certainty of 0.6000.

TABLE 40B
Encoded NOV40 protein sequence (SEQ ID NO:92).

MVNAPLCSFDIIVDGNSFGPCSSFELFADKVPKTVENFRALSTGGKGFGY
KGSCFHRIIPGFILSARVLTSHTIIMPQSIYQEKFDDENFILKHTGPGIL
SMANAGPDTNGSQFFTCVAKTEWLDGKHKVFGKVRRGVNIMEAMECSGSG
NGETGKKITTANCGQL

A search of sequence databases reveals that the NOV40 amino acid sequence has 122 of 166 amino acid residues (73%) identical to, and 131 of 166 amino acid residues (78%) similar to, the 165 amino acid residue ptnr:pir-id:CSHUA protein from human (peptidylprolyl isomerase (EC 5.2.1.8) A). Public amino acid databases include the GenBank databases, SwissProt, PDB and PIR.

NOV40 is expressed in at least Brain. Expression information was derived from the tissue sources of the sequences that were included in the derivation of the sequence of CG57157_{—}01. The sequence is predicted to be expressed in the following tissues because of the expression pattern of (GENBANK-ID: gb:GENBANK-ID:HSCYCR|acc:Y00052.1) a closely related Human mRNA for T-cell cyclophilin homolog in species Homo sapiens: Brain. This information was derived by determining the tissue sources of the sequences that were included in the invention including but not limited to SeqCalling sources, Public EST sources, Literature sources, and/or RACE sources.

The disclosed NOV40 polypeptide has homology to the amino acid sequences shown in the BLASTP data listed in Table 40C.

TABLE 40C
BLAST results for NOV40

| Gene Index/ | | Length | Identity | Positives | |
|---|---|---|---|---|---|
| Identifier | Protein/Organism | (aa) | (%) | (%) | Expect |
| gi\|14743515\|ref\|XP_ | hypothetical | 165 | 121/166 | 130/166 | e−59 |
| 017224.2\| | protein XP_017224 | | (72%) | (77%) | |
| (XM_017224) | [Homo sapiens] | | | | |
| gi\|12804335\|gb\|AAH03026.1\| | (protein for | 174 | 122/166 | 131/166 | e−59 |
| AAH03026 | IMAGE: 2823490) | | (73%) | (78%) | |
| (BC003026) | [Homo sapiens] | | | | |
| gi\|4033689\|sp\|P04374\| | PEPTIDYL-PROLYL | 164 | 122/166 | 131/166 | 4e−59 |
| CYPH_BOVIN | CIS-TRANS | | (73%) | (78%) | |
| | ISOMERASE A | | | | |
| | (PPIASE) | | | | |
| | (ROTAMASE) | | | | |
| | (CYCLOPHILIN A) | | | | |
| | (CYCLOSPORIN A- | | | | |
| | BINDING PROTEIN) | | | | |
| gi\|10863927\|ref\|NP_ | peptidylprolyl | 165 | 122/166 | 131/166 | 5e−59 |
| 066953.1\| | isomerase A | | (73%) | (78%) | |
| (NM_021130) | (cyclophilin A) | | | | |
| | [Homo sapiens] | | | | |
| gi\|68401\|pir\|\|CSBOAB | peptidylprolyl | 163 | 119/162 | 128/162 | 9e−59 |
| | isomerase (EC | | (73%) | (78%) | |
| | 5.2.1.8) A - | | | | |
| | bovine | | | | |

Table 40D lists the domain descriptions from DOMAIN analysis results against NOV40. This indicates that the NOV40 sequence has properties similar to those of other proteins known to contain this domain.

TABLE 40D
Domain Analysis of NOV40

gnl|Pfam|pfam00160, pro_isomerase, Cyclophilin type peptidyl-prolyl
cis-trans isomerase
CD-Length = 162 residues, 99.4% aligned
Score = 185 bits (469), Expect = 2e−48

The disclosed NOV40 nucleic acid of the invention encoding a Cis/Trans Peptidyl Prolyl Isomerase-like protein includes the nucleic acid whose sequence is provided in Table 40A or a fragment thereof. The invention also includes a mutant or variant nucleic acid any of whose bases may be changed from the corresponding base shown in Table 40A while still encoding a protein that maintains its Cis/Trans Peptidyl Prolyl Isomerase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 17 percent of the bases may be so changed.

The disclosed NOV40 protein of the invention includes the Cis/Trans Peptidyl Prolyl Isomerase-like protein whose sequence is provided in Table 40B. The invention also includes a mutant or variant protein any of whose residues may be changed from the corresponding residue shown in Table 40B while still encoding a protein that maintains its Cis/Trans Peptidyl Prolyl Isomerase-like activities and physiological functions, or a functional fragment thereof. In the mutant or variant protein, up to about 27 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Cis/Trans Peptidyl Prolyl Isomerase-like protein (NOV40) may function as a member of a “Cis/Trans Peptidyl Prolyl Isomerase family”. Therefore, the NOV40 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV40 nucleic acids and proteins of the invention are useful in potential therapeutic applications implicated in diseases including but not limited to various pathologies and disorders as indicated below. For example, a cDNA encoding the Cis/Trans Peptidyl Prolyl Isomerase-like protein (NOV40) may be useful in gene therapy, and the Cis/Trans Peptidyl Prolyl Isomerase-like protein (NOV40) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV40 nucleic acid encoding the Cis/Trans Peptidyl Prolyl Isomerase-like protein of the invention, or fragments thereof, may further be useful in diagnostic applications, wherein the presence or amount of the nucleic acid or the protein are to be assessed.

NOV40 nucleic acids and polypeptides are further useful in the generation of antibodies that bind immuno-specifically to the novel NOV40 substances for use in therapeutic or diagnostic methods. These antibodies may be generated according to methods known in the art, using prediction from hydrophobicity charts, as described in the “Anti-NOVX Antibodies” section below. The disclosed NOV40 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV41

A disclosed NOV41 nucleic acid of 525 nucleotides (also referred to as CG57159-01) encoding a Cis/Trans Peptidyl Prolyl Isomerase-like protein is shown in Table 41A. The start and stop codons are in bold letters.

TABLE 41A
NOV41 nucleotide sequence (SEQ ID NO:93).

GCCCAGAACTCCCTGCCACCAGCCATGGCCAACCCCACTGTGTTCTTCAA
CATTGCAATTGATAGTGAGTCCTTGGGCTGCATCTCCTTCAAGCTATTTG
CAGACAAAGTTCTAAAGATGGAAGAAAATTTTTGTGCTCTGAACACTGGA
GAGAAAGTATTTGGTGATAAATGTCCCTGCTTTTACAGAATTATTCCGGG
GGTGTGTCAGGGTGGTGACTTCACACACCATAATGGCACTGGTGGCAAGT
CCCTCTACAGCAAGGAATTTGATGATGAGAACTTCATCCTAAAGCATACA
GCTCCTGGCGTCTTGTCCACGGCAAATGCTGGACCCACCACAAATGGTTC
CCAGTTTTTCTTCTGTACTGCCAAGACAGAGGATGGACAGCATGTGGTCT
TTGGCAAGGTGAAAGATGGCATGAGTATTGTGGAAGCCCTGGAACGCTCT
GGGTCCAGGAATGGTAAGACCAGCAAGAAGATCACAGCTGCTGACTGTGG
ACAACTCTAATAAATTTGATTGTTT

In a search of public sequence databases, the NOV41 nucleic acid sequence, located on chromsome 11 has 442 of 515 bases (85%) identical to a gb:GENBANK-ID:HSCYCR|acc:Y00052.1 mRNA from Homo sapiens (Human mRNA for T-cell cyclophilin). Public nucleotide databases include all GenBank databases and the GeneSeq patent database.

The disclosed NOV41 polypeptide (SEQ ID NO:94) encoded by SEQ ID NO:93 has 161 amino acid residues and is presented in Table 41B using the one-letter amino acid code. Signal P, Psort and/or Hydropathy results predict that NOV41 has no signal peptide and is likely to be localized in the plasma membrane with a certainty of 0.600.

TABLE 41B
Encoded NOV41 protein sequence (SEQ ID NO:94).

MANPTVFFNIAIDSESLGCISFKLFADKVLKMEENFCALNTGEKVFGDKC
PCFYRIIPGVCQGGDFTHHNGTGGKSLYSKEFDDENFILKHTAPGVLSTA
NAGPTTNGSQFFFCTAKTEDGQHVVFGKVKDGMSIVEALERSGSRNGKTS
KKITAADCGQL

A search of sequence databases reveals that the NOV41 amino acid sequence has 125 of 164 amino acid residues (76%) identical to, and 141 of 164 amino acid residues (85%) similar to, the 165 amino acid residue ptnr:pir-id:CSHUA protein from human (peptidylprolyl isomerase (EC 5.2.1.8) A). Public amino acid databases include the GenBank databases, SwissProt, PDB and PIR.

NOV41 is expressed in at least Heart, Placenta, Stomach, Whole Organism. Expression information was derived from the tissue sources of the sequences that were included in the derivation of the sequence of CG57159_-o The sequence is predicted to be expressed in the following tissues because of the expression pattern of (GENBANK-ID: gb:GENBANK-ID:HSCYCR|acc:Y00052.1) a closely related Human mRNA for T-cell cyclophilin homolog in species Homo sapiens: signet-ring cell carcinoma cell_line. This information was derived by determining the tissue sources of the sequences that were included in the invention including but not limited to SeqCalling sources, Public EST sources, Literature sources, and/or RACE sources.

The disclosed NOV41 polypeptide has homology to the amino acid sequences shown in the BLASTP data listed in Table 41C.

TABLE 41C
BLAST results for NOV41

| Gene Index/ | | Length | Identity | Positives | |
|---|---|---|---|---|---|
| Identifier | Protein/Organism | (aa) | (%) | (%) | Expect |
| gi\|12804335\|gb\| | (protein for | 174 | 125/164 | 141/164 | 3e−53 |
| AAH03026.1\| | IMAGE: 2823490) | | (76%) | (85%) | |
| AAH03026 | [Homo sapiens] | | | | |
| (BC003026) | | | | | |
| gi\|4033689\|sp\|P04374\| | PEPTIDYL-PROLYL | 164 | 125/164 | 141/164 | 9e−53 |
| CYPH_BOVIN | CIS-TRANS | | (76%) | (85%) | |
| | ISOMERASE A | | | | |
| | (PPIASE) | | | | |
| | (ROTAMASE) | | | | |
| | (CYCLOPHILIN A) | | | | |
| | (CYCLOSPORIN A- | | | | |
| | BINDING PROTEIN) | | | | |
| gi\|13937981\|gb\| | peptidylprolyl | 165 | 125/164 | 141/164 | 9e−53 |
| AAH07104.1\| | isomerase A | | (76%) | (85%) | |
| AAH07104 | (cyclophilin A) | | | | |
| (BC007104) | [Homo sapiens] | | | | |
| gi\|10863927\|ref\|NP_ | peptidylprolyl | 165 | 125/164 | 141/164 | 1e−52 |
| 066953.1\| | isomerase A | | (76%) | (85%) | |
| (NM_021130) | (cyclophilin A) | | | | |
| | [Homo sapiens] | | | | |
| gi\|68401\|pir\|\|CSBOAB | peptidylprolyl | 163 | 124/162 | 140/162 | 4e−52 |
| | isomerase (EC | | (76%) | (85%) | |
| | 5.2.1.8) A - | | | | |
| | bovine | | | | |

Table 41D lists the domain descriptions from DOMAIN analysis results against NOV41. This indicates that the NOV41 sequence has properties similar to those of other proteins known to contain this domain.

TABLE 41D
Domain Analysis of NOV41

gnl|Pfam|pfam00160, pro_isomerase, Cyclophilin type peptidyl-prolyl
cis-trans isomerase
CD-Length = 162 residues, 100.0% aligned
Score = 177 bits (448), Expect = 5e−46

The disclosed NOV41 nucleic acid of the invention encoding a Cis/Trans Peptidyl Prolyl Isomerase-like protein includes the nucleic acid whose sequence is provided in Table 41A or a fragment thereof. The invention also includes a mutant or variant nucleic acid any of whose bases may be changed from the corresponding base shown in Table 41A while still encoding a protein that maintains its Cis/Trans Peptidyl Prolyl Isomerase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 15 percent of the bases may be so changed.

The disclosed NOV41 protein of the invention includes the Cis/Trans Peptidyl Prolyl Isomerase-like protein whose sequence is provided in Table 41B. The invention also includes a mutant or variant protein any of whose residues may be changed from the corresponding residue shown in Table 41B while still encoding a protein that maintains its Cis/Trans Peptidyl Prolyl Isomerase-like activities and physiological functions, or a functional fragment thereof. In the mutant or variant protein, up to about 24 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Cis/Trans Peptidyl Prolyl Isomerase-like protein (NOV41) may function as a member of a “Cis/Trans Peptidyl Prolyl Isomerase family”. Therefore, the NOV41 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV41 nucleic acids and proteins of the invention are useful in potential therapeutic applications implicated in diseases including but not limited to various pathologies and disorders as indicated below. For example, a cDNA encoding the Cis/Trans Peptidyl Prolyl Isomerase-like protein (NOV41) may be useful in gene therapy, and the Cis/Trans Peptidyl Prolyl Isomerase-like protein (NOV41) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV41 nucleic acid encoding the Cis/Trans Peptidyl Prolyl Isomerase-like protein of the invention, or fragments thereof, may further be useful in diagnostic applications, wherein the presence or amount of the nucleic acid or the protein are to be assessed.

NOV41 nucleic acids and polypeptides are further useful in the generation of antibodies that bind immuno-specifically to the novel NOV41 substances for use in therapeutic or diagnostic methods. These antibodies may be generated according to methods known in the art, using prediction from hydrophobicity charts, as described in the “Anti-NOVX Antibodies” section below. The disclosed NOV41 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV42

NOV42 includes two Cis/Trans Peptidyl Prolyl Isomerase-like proteins disclosed below. The disclosed sequences have been named NOV42a and NOV42b.

NOV42a

A disclosed NOV42a nucleic acid of 720 nucleotides (also referred to as CG57226-01) encoding a Cis/Trans Peptidyl Prolyl Isomerase-like protein is shown in Table 42A. The start and stop codons are in bold letters.

TABLE 42A
NOV42a nucleotide sequence (SEQ ID NO:95).

CATCAGGAAAATGCAAATCAAACCACAACGAGATATCATGTCACACCAAT
TAGGATGGCCACTATTAAAAACATAAAATTAATAAGCATTGGCAAGGATG
TAGAAATTAGAACACCTGTGCACTGTTGGTGGGAATATAAAATGATGCAG
CTGGCTTTGCAGACACTGCTGTCCCCCAACACCCCCTGTCACTAGGCCAT
GGTCATCCCGACTGTGCCCTTCAACATCACCATCAACAGCAAGCCCTTAG
GACACATCTCCTTTCAGCTATTTGCAGACAAATTTCCAAAGACAGGAGAA
AACTTTCACACTCTGAACAATAAAGACAAAGGATTTGGTTCCTGCTTTCA
CAGAATTATTCCGGAGTTTATATGCCAGGGTGATGACTTCACACCCCATA
ATGGCATTGGTGGCAAGTCCATCTACGGGGATAAATTTGATGATAAGAAC
TTTATTGTGAAGCATACAGGTCTTGGCATCTTGTCCATGGCAAATGCTGC
ACCCAAAACAAATGAGTCCCAGTTTTTCATCTGCACTGCCATGGCCAAAT
GGTGGGATGGCAAGCATGTGATCTTTGGCAGGGTGAAAGAGGGCATGAAT
ATTGTGGAAGCCATGGAATGCTTTGGGTCCAGGAATGGCAAGACAAGCAA
GATCGCCATTGCCAACTGCAGACAACTCTGATAAATTTGACTTGTGTTTT
ATCTTAACCACCAGACCTTT

In a search of public sequence databases, the NOV42a nucleic acid sequence, located on chromsome 11 has 338 of 387 bases (87%) identical to a gb:GENBANK-ID:AK026569|acc:AK026569.1 mRNA from Homo sapiens (Homo sapiens cDNA: FLJ22916 fis, clone KAT06406, highly similar to HSCYCR Human mRNA for T-cell cyclophilin). Public nucleotide databases include all GenBank databases and the GeneSeq patent database.

The disclosed NOV42a polypeptide (SEQ ID NO:96) encoded by SEQ ID NO:95 has 160 amino acid residues and is presented in Table 42B using the one-letter amino acid code. Signal P, Psort and/or Hydropathy results predict that NOV42a has no signal peptide and is likely to be localized in the microbody with a certainty of 0.6400.

TABLE 42B
Encoded NOV42a protein sequence (SEQ ID NO:96).

MVIPTVPFNITINSKPLGHISFQLFADKFPKTGENFHTLNNKDKGFGSCF
HRIIPEFICQGDDFTPHNGIGGKSIYGDKFDDKNFIVKHTGLGILSMANA
APKTNESQFFICTAMAKWWDGKHVIFGRVKEGMNIVEAMECFGSRNGKTS
KIAIANCRQL

A search of sequence databases reveals that the NOV42a amino acid sequence has 118 of 164 amino acid residues (71%) identical to, and 135 of 164 amino acid residues (82%) similar to, the 165 amino acid residue ptnr:pir-id:CSHUA protein from human (peptidylprolyl isomerase (EC 5.2.1.8) A). Public amino acid databases include the GenBank databases, SwissProt, PDB and PIR.

NOV42a is expressed in at least Brain and Peripheral Blood. Expression information was derived from the tissue sources of the sequences that were included in the derivation of the sequence of CG57226_{—}01. The sequence is predicted to be expressed in the following tissues because of the expression pattern of (GENBANK-ID: gb:GENBANK-ID:AK026569|acc:AK026569.1) a closely related Homo sapiens cDNA: FLJ22916 fis, clone KAT06406, highly similar to HSCYCR Human mRNA for T-cell cyclophilin homolog in species Homo sapiens: signet-ring cell carcinoma cell_line. This information was derived by determining the tissue sources of the sequences that were included in the invention including but not limited to SeqCalling sources, Public EST sources, Literature sources, and/or RACE sources.

NOV42b

A disclosed NOV42b nucleic acid of 600 nucleotides (also referred to as CG57226-02) encoding a Cis/Trans Peptidyl Prolyl Isomerase-like protein is shown in Table 42C. The start and stop codons are in bold letters.

TABLE 42C
NOV42 nucleotide sequence (SEQ ID NO:97).

CTGTGCACTGTTGGTGGGAATATAAAATGATGCAGCTGGCTTTGCAGACA
CTGCTGTCCCCCAACACCCCCTGTCACTAGGCCATGGTCATCCCGACTGT
GCCCTTCAACATCACCATCAACAGCAAGCCCTTAGGACACATCTCCTTTC
AGCTATTTGCAGACAAATTTCCAAAGACAGGAGAAAACTTTCACACTCTG
AACAATAAAGACAAAGGATTTGGTTCCTGCTTTCACAGAATTATTCCGGA
GTTTATATGCCAGGGTGATGACTTCACACCCCATAATGGCATTGGTGGCA
AGTCCATCTACGGGGATAAATTTGATGATAAGAACTTTATTGTGAAGCAT
ACAGGTCTTGGCATCTTGTCCATGGCAAATGCTGCACCCAAAACAAATGA
GTCCCAGTTTTTCATCTGCACTGCCATGGCCAAATGGTGGGATGGCAAGC
ATGTGATCTTTGGCAGGGTGAAAGAGGGCATGAATATTGTGGAAGCCATG
GAATGCTTTGGGTCCAGGAATGGCAAGACAAGCAAGATCGCCATTGCCAA
CTGCAGACAACTCTGATAAATTTGACTTGTGTTTTATCTTAACCACCAGA

In a search of public sequence databases, the NOV42b nucleic acid sequence, located on chromsome 11 has 335 of 382 bases (87%) identical to a gb:GENBANK-ID:AK026569|acc:AK026569.1 mRNA from Homo sapiens (Homo sapiens cDNA: FLJ22916 fis, clone KAT06406, highly similar to HSCYCR Human mRNA for T-cell cyclophilin). Public nucleotide databases include all GenBank databases and the GeneSeq patent database.

The disclosed NOV42b polypeptide (SEQ ID NO:98) encoded by SEQ ID NO:97 has 160 amino acid residues and is presented in Table 42D using the one-letter amino acid code. Signal P, Psort and/or Hydropathy results predict that NOV42b has no signal peptide and is likely to be localized in the microbody with a certainty of 0.6400.

TABLE 42D
Encoded NOV42b protein sequence (SEQ ID NO:98).

MVIPTVPFNITINSKPLGHISFQLFADKFPKTGENFHTLNNKDKGFGSCF
HRIIPEFICQGDDFTPHNGIGGKSIYGDKFDDKNFIVKHTGLGILSMANA
APKTNESQFFICTAMAKWWDGKHVIFGRVKEGMNIVEAMECFGSRNGKTS
KIAIANCRQL

A search of sequence databases reveals that the NOV42b amino acid sequence has 118 of 164 amino acid residues (71%) identical to, and 135 of 164 amino acid residues (82%) similar to, the 165 amino acid residue ptnr:pir-id:CSHUA protein from human (peptidylprolyl isomerase (EC 5.2.1.8) A). Public amino acid databases include the GenBank databases, SwissProt, PDB and PIR.

NOV42b is expressed in at least Brain, and Peripheral Blood. This information was derived by determining the tissue sources of the sequences that were included in the invention including but not limited to SeqCalling sources, Public EST sources, Literature sources, and/or RACE sources.

The disclosed NOV42a polypeptide has homology to the amino acid sequences shown in the BLASTP data listed in Table 42E.

TABLE 42E
BLAST results for NOV42a

| Gene Index/ | | Length | Identity | Positives | |
|---|---|---|---|---|---|
| Identifier | Protein/Organism | (aa) | (%) | (%) | Expect |
| gi\|12804335\|gb\|AAH03026.1\| | protein for | 174 | 118/164 | 135/164 | 1e−60 |
| AAH03026 | IMAGE: 2823490) [Homo | | (71%) | (81%) | |
| (BC003026) | sapiens ] | | | | |
| gi\|10863927\|ref\|NP_ | peptidylprolyl isomerase | 165 | 118/164 | 135/164 | 8e−60 |
| 066953.1\| | A (cyclophilin A) [Homo | | (71%) | (81%) | |
| (NM_021130) | sapiens ] | | | | |
| gi\|4033689\|sp\|P04374\| | PEPTIDYL-PROLYL | 164 | 118/164 | 135/164 | 8e−60 |
| CYPH_BOVIN | CIS-TRANS | | (71%) | (81%) | |
| | ISOMERASE A | | | | |
| | (PPIASE) (ROTAMASE) | | | | |
| | (CYCLOPHILIN A) | | | | |
| | (CYCLOSPORIN A- | | | | |
| | BINDING PROTEIN) | | | | |
| gi\|13937981\|gb\|AAH07104.1\| | peptidylprolyl isomerase | 165 | 118/164 | 135/164 | 9e−60 |
| AAH07104 | A (cyclophilin A) [Homo | | (71%) | (81%) | |
| (BC007104) | sapiens ] | | | | |
| gi\|14743515\|ref\|XP_ | hypothetical protein | 165 | 118/164 | 135/164 | 3e−59 |
| 017224.2\| | XP_017224 [Homo | | (71%) | (81%) | |
| (XM_017224) | sapiens ] | | | | |

Table 42F lists the domain descriptions from DOMAIN analysis results against NOV 42. This indicates that the NOV42 sequence has properties similar to those of other proteins known to contain this domain.

TABLE 42F
Domain Analysis of NOV42

gnl|Pfam|pfam00160, pro_isomerase, Cyclophilin type peptidyl-prolyl
cis-trans isomerase.
CD-Length = 162 residues, 100.0% aligned
Score = 185 bits (469), Expect = 2e−48

The disclosed NOV42 nucleic acid of the invention encoding a Cis/Trans Peptidyl Prolyl Isomerase-like protein includes the nucleic acid whose sequence is provided in Table 42A or 42C or a fragment thereof. The invention also includes a mutant or variant nucleic acid any of whose bases may be changed from the corresponding base shown in Table 42A or 42C while still encoding a protein that maintains its Cis/Trans Peptidyl Prolyl Isomerase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In the mutant or variant nucleic acids, and their complements, up to about 13 percent of the bases may be so changed.

The disclosed NOV42 protein of the invention includes the Cis/Trans Peptidyl Prolyl Isomerase-like protein whose sequence is provided in Table 42B or 42D. The invention also includes a mutant or variant protein any of whose residues may be changed from the corresponding residue shown in Table 42B or 42D while still encoding a protein that maintains its Cis/Trans Peptidyl Prolyl Isomer-ase-like activities and physiological functions, or a functional fragment thereof. In the mutant or variant protein, up to about 29 percent of the residues may be so changed. The invention further encompasses antibodies and antibody fragments, such as F_{ab} or (F_{ab})₂, that bind immunospecifically to any of the proteins of the invention.

The above defined information for this invention suggests that this Cis/Trans Peptidyl Prolyl isomerase-like protein (NOV42) may function as a member of a “Cis/Trans Peptidyl Prolyl Isomerase family”. Therefore, the NOV42 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV42 nucleic acids and proteins of the invention are useful in potential therapeutic applications implicated in diseases including but not limited to various pathologies and disorders as indicated below. For example, a cDNA encoding the Cis/Trans Peptidyl Prolyl Isomerase-like protein (NOV42) may be useful in gene therapy, and the Cis/Trans Peptidyl Prolyl Isomerase-like protein (NOV42) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV42 nucleic acid encoding the Cis/Trans Peptidyl Prolyl Isomerase-like protein of the invention, or fragments thereof, may further be useful in diagnostic applications, wherein the presence or amount of the nucleic acid or the protein are to be assessed.

NOV42 nucleic acids and polypeptides are further useful in the generation of antibodies that bind immuno-specifically to the novel NOV42 substances for use in therapeutic or diagnostic methods. These antibodies may be generated according to methods known in the art, using prediction from hydrophobicity charts, as described in the “Anti-NOVX Antibodies” section below. The disclosed NOV42 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV43

A disclosed NOV43 nucleic acid of 3146 nucleotides (also referred to as CG57538-01) encoding a ceruloplasmin-like protein is shown in Table 43A. The start and stop codons are in bold letters.

TABLE 43A
NOV43 nucleotide sequence (SEQ ID NO:99).

AATTTGAAAATGAAGGCACTTTTACCATTGACCTTTCTGTTTTTTATTAGTTCTCCAGGTTGGGCAATAG
ATAGGCACTGCTACATAGGCATTGAAGAAAGCATTTGGAACTATGCTAATGCTGATGAAAACTTTCTCAT
GATTGACACTTGCAGGACACATATGCCATTATTTCTACAAGGAGGTCAAGCGAGGAAGAGCTTTGTTTTT
AAAAAGGCTTTGTATTTTCAATATACTGATAATACATTTCAAAGGATCATTGAAAAACCATCCTGGTTGG
GATTTTTAGGTCCAATGATTAAAGCAGAGACTGGAGACTTCATTTATGTACATGTAAAAAATAATGCTTC
AAGAGCTTATAGTTATCATCCTCATGGGCTCACCTACTCCAAAGAAAATGAAGGTGCTATCTATCCTGAT
AATACGACAGGCCTGCAAAAGGAAGATGAATATCTGGAGCCAGGGAAACAATATACCTACAAGTGGTATG
TAGAAGAACATCAGGGACCTGGCCCCAATGACAGTAATTGTGTGACAAGAATTTACCATTCCCATATAGA
CACTGCAAGAGATGTAGCTTCGGGACTTATTGGACCAATACTGACTTGTAAAAGAGGTACACTGAATGGA
GACACTGAAAAAGATATTGACAGGTCTTCTTTTCTGATGTTTTCTACAACTGATGAAAGCAGAAGCTGGT
ATAGTGATGAAAATATTCGTGCATTTACTGAATCTGGCAAGATTAATACTAGTGATCCCCGTTTTGAGGA
GAGCATGAGCATGCAAGCAATAAATGGATACATCTATGGAAATCTGCCCAATCTCACCATGTGTGCTGAA
GATAGGGTCCAGTGGTATTTTGTTGGCATGGGTGGCGTGGCTGACATACACCCCGTCTACCTCCGCGGAC
AAACTCTGATCTCTCGGAATCACAGAAAGGACACCATTATGCTCTTCCCCTCCTCACTGCAAGATGCCTT
CATGGTGGCCAAGGCCCCTGGAGTGTGGATGCTGGGATGCCAGATGCAGGCATTTTTCAAAGTAAGTAAT
TGCCAGAAACCTTCAACAGAAGCCTTTGTTACTGGGACACATGTTATACATTACTATATTGCTGCTAAAG
AAATTCTTTGGAACTATGCTCCATCTGGTATAGATTTCTTCACTAAAAAAAATTTAACAGCAGCTGGAAG
TAAATCCCAGTTATTTTTTGAACGAACTCCAACCAGAATTGGAGGAACTAACAAAAAACTGATTTACCGT
GAATACACAGATGCTTCCTTCCAAACACAGAAGGCAAGAGAAGAACACCTTGGAATCCTAGGCCCCGTTA
TTAAGGCAGAGGTGAGACAGACCATCAAAATCACTTTCTATAACAATGCTTCCCTGCCACTCAGCATTCA
GCCTCCTGGACTGCATTACAACAAGAGCTTGTGGCAGAGTTATTACTTTAGTTCCTATTCAACTGTCACC
CAAAGAGAAAGATCTGTTCCTCCACCCTCTTCACATGTAAGTCCTGGCACAACATTTGTCTATACATGGG
AAGTTCCAAAAGATGTGGGTCCCACCTCCACAGATCCCAACTGCTTGACCTGGTTCTATTACTCTTCAGT
AAATGGGAAAAAAGACATCAACAGTGGCCTTCTGGGGCCTCTCCTTATATGTAGAAATGGAAGTCTTGGA
GACGATGGCAAACAGAAAGGAGTAGACAAAGAGTTTTACCTACTTGCCACAATATTTGATGAAAATGAAA
GTAATCTCTTGGATGAAAATATCAGAACATTTATCACAGAGCCTGAAAACATAGATAAAGAGGATACAGA
CTGCCAAGCCTCAAATAAGATGTACGCCATAAATGGATACATGTATGGAAATCTGCCTGGATTGGACACG
TGCTTAGGAGAGAACGTTTTGTGGCACGTTTTTAGTGTAGGATCAGTGGAAGATTTACACGGGATATATT
TTTCAGGAAATACCTTCACTTCTTTAGGAGCAAGAAGGGACACAATACCTATGTTTCCTTATACTTCTCA
GACGCTTTTGATGACACCTGATTCTATAGGTACTTTTGATTTGGTTTGCATGACAATAAAGCACAATCTA
GGAGGCATGAAACATAAATATCACGTGAGGCAATGTGGGAAGCCAAACCCTGATCAAACACAATACCAGG
AGGAGAAAATAATTATTACCATTGCAGCCGAGGAAATGGAATGGGATTATTCTCCTAGTAGAAAGTGGGA
GAATGAACTCCACCACTTACGAAGAGAGAGCCAAACGAGCATGTATGTGGACAGAAGTGGAACACTTCTT
GGGTCCAAATACAAGAAAGTCTTATATCGTCAATATGATGATAACACGTCACAAATCAAACAAAAAGGAA
TGAGGGTGAAAAACATCTCGATACTAGGTCCATTAATATTGCTCAACCCTGGTCAAATAATTCAAATTAT
CTTTAAAAATAAAGCCGCAAGACCGTATTCTATTCATGCTCATGGAGTGAAAACAAATAATTCCACTGTT
GTTCCAACTCAGCCAGGTGAGATTCAAATATATACTTGGCAGATACCTGATAGAACTGGTCCTACCTCAC
TGGACTTTGAATGCATACCTTGGTTTTACTATTCAACTGTATCTGTGGCTAAGGACCTTCACAGTGGACT
GGTAGGCCCTCTCTCTGTATGCCGCAAAGACATCAACCCCAACATAGTTCACCGTGTTCTCCACTTCATG
ATATTTGATGAGAATGAATCCTGGTACTTCGAAGACAGTATCAACACCTATGCTTCAAAACCAAACAAAG
TGGACAAGGAAAATGATAATTTTCAACTCAGCAACCAAATGCACGCAATTAACGGAAGACTGTTTGGAAA
TAACCAAGGTATAACATTCCATGTTGGGGATGTAGTGAATTGGTATCTGATTGGCATAGGGAATGAAGCT
GGAGTGTATCAATCTGATGTTTATGACCTTCCTCCTGGGGTCTATCGAACTGTAAAAATGTATCGAAGAG
ATGTTGGAACCTGGTTATTTTATTGCCATGTTTTTGAGCACATTGGTGCTGGAATGGAAAGCACTTACAC
TGTACTTGAAAGAAAAGGTAAGATCCATTGGCTAAATTAATTAGAAGTGATATTTAAACAAATGCA

In a search of public sequence databases, the NOV43 nucleic acid sequence, located on chromsome 3 has 1113 of 1697 bases (65%) identical to a gb:GENBANK-ID:HUMCERP|acc:M13699.1 mRNA from Homo sapiens (Human ceruloplasmin (ceruloplasmin) mRNA, complete cds). Public nucleotide databases include all GenBank databases and the GeneSeq patent database.

The disclosed NOV43 polypeptide (SEQ ID NO:100) encoded by SEQ ID NO:99 has 1036 amino acid residues and is presented in Table 43B using the one-letter amino acid code. Signal P, Psort and/or Hydropathy results predict that NOV43 has a signal peptide and is likely to be localized extracellularly with a certainty of 0.4085. The most likely cleavage site for a NOV43 peptide is between amino acids 19 and 20.

TABLE 43B
Encoded NOV43 protein sequence (SEQ ID NO:100).

MKALLPLTFLFFISSPGWAIDRHCYIGIEESIWNYANADENFLMIDTCRT
HMPLFLQGGQARKSFVFKKALYFQYTDNTFQRIIEKPSWLGFLGPMIKAE
TGDFIYVHVKNNASRAYSYHPHGLTYSKENEGAIYPDNTTGLQKEDEYLE
PGKQYTYKWYVEEHQGPGPNDSNCVTRIYHSHIDTARDVASGLIGPILTC
KRGTLNGDTEKDIDRSSFLMFSTTDESRSWYSDENIRAFTESGKINTSDP
RFEESMSMQAINGYIYGNLPNLTMCAEDRVQWYFVGMGGVADIHPVYLRG
QTLISRNHRKDTIMLFPSSLEDAFMVAKAPGVWMLGCQMQAFFKVSNCQK
PSTEAFVTGTHVIHYYIAAKEILWNYAPSGIDFFTKKNLTAAGSKSQLFF
ERSPTRIGGTNKKLIYREYTDASFQTQKAREEHLGILGPVIKAEVRQTIK
ITFYNNASLPLSIQPPGLHYNKSLWQSYYFSSYSTVTQRERSVPPPSSHV
SPGTTFVYTWEVPKDVGPTSTDPNCLTWFYYSSVNGKKDINSGLLGPLLI
CRNGSLGDDGKQKGVDKEFYLLATIFDENESNLLDENIRTFITEPENIDK
EDTDCQASNKMYAINGYMYGNLPGLDTCLGDNVLWHVFSVGSVEDLHGIY
FSGNTFTSLGARRDTIPMFPYTSQTLLMTPDSIGTFDLVCMTIKHNLGGM
KHKYHVRQCGKPNPDQTQYQEEKIIITIAAEEMEWDYSPSRKWENELHHL
RRESQTSMYVDRSGTLLGSKYKKVLYRQYDDNTSQIKQKGMRVKNISILG
PLILLNPGQIIQIIFKNKAARPYSIHAHGVKTNNSTVVPTQPGEIQIYTW
QIPDRTGPTSLDFECIPWFYYSTVSVAKDLHSGLVGPLSVCRKDINPNIV
HRVLHFMIFDENESWYFEDSINTYASKPNKVDKENDNFQLSNQMHAINGR
LFGNNQGITFHVGDVVNWYLIGIGNEAGVYQSDVYDLPPGVYRTVKMYRR
DVGTWLFYCHVFEHIGAGMESTYTVLERKGKIHWLN

A search of sequence databases reveals that the NOV43 amino acid sequence has 548 of 994 amino acid residues (55%) identical to, and 719 of 994 amino acid residues (72%) similar to, the 1069 amino acid residue ptnr:pir-id:KUHU protein from human (ceruloplasmin (EC 1.16.3.1) precursor [validated]). Public amino acid databases include the GenBank databases, SwissProt, PDB and PIR.

NOV43 is expressed in at least salivary glands. Expression information was derived from the tissue sources of the sequences that were included in the derivation of the sequence of CG57538-01. The sequence is predicted to be expressed in the following tissues because of the expression pattern of (GENBANK-ID: gb:GENBANK-ID:HUMCERP|acc:M13699.1) a closely related Human ceruloplasmin (ceruloplasmin) mRNA, complete cds homolog in species Homo sapiens:liver, secreted into plasma. This information was derived by determining the tissue sources of the sequences that were included in the invention including but not limited to SeqCalling sources, Public EST sources, Literature sources, and/or RACE sources.

The disclosed NOV43 polypeptide has homology to the amino acid sequences shown in the BLASTP data listed in Table 43C.

TABLE 43C
BLAST results for NOV43

| Gene Index/ | | Length | Identity | Positives | |
|---|---|---|---|---|---|
| Identifier | Protein/Organism | (aa) | (%) | (%) | Expect |
| gi\|1070458\|pir\|\|KUHU | ferroxidase (EC | 1069 | 579/1068 | 760/1068 | 0.0 |
| | 1.16.3.1) | | (54%) | (70%) | |
| | precursor - human | | | | |
| gi\|4557485\|ref\|NP_000087.1\| | ceruloplasmin | 1065 | 578/1066 | 758/1066 | 0.0 |
| (NM_000096) | (ferroxidase); | | (54%) | (70%) | |
| | Ceruloplasmin | | | | |
| | [Homo sapiens] | | | | |
| gi\|1942284\|pdb\|1KCW\| | X-Ray Crystal | 1048 | 568/1046 | 746/1046 | 0.0 |
| | Structure of | | (54%) | (71%) | |
| | Human | | | | |
| | Ceruloplasmin At | | | | |
| | 3.0 Angstroms | | | | |
| gi\|5281319\|gb\|AAD41477.1\| | (AF134814) | 1048 | 577/1054 | 742/1054 | 0.0 |
| AF134814_1 | ceruloplasmin | | (54%) | (69%) | |
| (AF134814) | [Ovis aries] | | | | |
| gi\|6680997\|ref\|NP_031778.1\| | gi\|6680997\|ref\|NP_{—} | 1062 | 560/1062 | 737/1062 | 0.0 |
| (NM_007752) | 031778.1\| | | (52%) | (68%) | |
| | (NM_007752) | | | | |

Table 43D lists the domain descriptions from DOMAIN analysis results against NOV43. This indicates that the NOV43 sequence has properties similar to those of other proteins known to contain this domain.

TABLE 43D
Domain Analysis of NOV43

gnl|Pfam|pfam00394, Cu-oxidase, Multicopper oxidase. Many of the
proteins in this family contain multiple similar copies of this
plastocyanin-like domain.
CD-Length = 135 residues, 90.4% aligned
Score = 37.7 bits (86), Expect = 0.003

In Wilson disease, the basal ganglia and liver undergo changes that express themselves in neurologic manifestations and signs of cirrhosis, respectively. A disturbance in copper metabolism is somehow involved in the mechanism. Low ceruloplasmin is found in the serum. Shokeir and Shreffler (1969) advanced the hypothesis that ceruloplasmin functions in enzymatic transfer of copper to copper-containing enzymes such as cytochrome oxidase. Supporting the hypothesis was the finding of markedly reduced levels of activity of cytochrome oxidase in Wilson disease and moderate reductions in heterozygotes. An abnormality of ceruloplasmin seems to be involved in Wilson disease. The fact that individuals with hereditary ceruloplasmin deficiency have profound iron accumulation in most tissues suggests that ceruloplasmin is important for normal release of cellular iron (Mukhopadhyay et al., 1998). At least 3 variants determined by codominant alleles have been identified by starch gel electrophoresis (Shreffler et al., 1967). Human ceruloplasmin is composed of a single polypeptide chain (Takahashi et al., 1984).

The disclosed NOV43 nucleic acid of the invention encoding a ceruloplasmin-like protein includes the nucleic acid whose sequence is provided in Table 43A or a fragment thereof. The invention also includes a mutant or variant nucleic acid any of whose bases may be changed from the corresponding base shown in Table 43A while still encoding a protein that maintains its ceruloplasmin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 35 percent of the bases may be so changed.

The disclosed NOV43 protein of the invention includes the ceruloplasmin-like protein whose sequence is provided in Table 43B. The invention also includes a mutant or variant protein any of whose residues may be changed from the corresponding residue shown in Table 43B while still encoding a protein that maintains its ceruloplasmin-like activities and physiological functions, or a functional fragment thereof. In the mutant or variant protein, up to about 45 percent of the residues may be so changed.

The invention further encompasses antibodies and antibody fragments, such as F_{ab} or (F_{ab})₂, that bind immunospecifically to any of the proteins of the invention.

The above defined information for this invention suggests that this ceruloplasmin-like protein (NOV43) may function as a member of a “ceruloplasmin family”. Therefore, the NOV43 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV43 nucleic acids and proteins of the invention are useful in potential therapeutic applications implicated in diseases including but not limited to various pathologies and disorders as indicated below. For example, a cDNA encoding the ceruloplasmin-like protein (NOV43) may be useful in gene therapy, and the ceruloplasmin-like protein (NOV43) may be useful when administered to a subject in need thereof. By way of nonlimiting example, the compositions of the present invention will have efficacy for treatment of patients suffering from Wilson disease, dementia, diabetes, retinal degeneration, neurologic degeneration, xerostomia, or other pathologies or conditions. The NOV43 nucleic acid encoding the ceruloplasmin-like protein of the invention, or fragments thereof, may further be useful in diagnostic applications, wherein the presence or amount of the nucleic acid or the protein are to be assessed.

NOV43 nucleic acids and polypeptides are further useful in the generation of antibodies that bind immuno-specifically to the novel NOV43 substances for use in therapeutic or diagnostic methods. These antibodies may be generated according to methods known in the art, using prediction from hydrophobicity charts, as described in the “Anti-NOVX Antibodies” section below. The disclosed NOV43 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV44

NOV44 includes two Leucine rich repeat-like proteins disclosed below. The disclosed sequences have been named NOV44a and NOV44b.

NOV44a

A disclosed NOV44a nucleic acid of 857 nucleotides (also referred to as CG57623-01) encoding a Leucine rich repeat-like protein is shown in Table 44A. The start and stop codons are in bold letters.

TABLE 44A
NOV44a nucleotide sequence.

(SEQ ID NO:101)

AACAATAAACTTCTAACACTCCCTTACTAAAAGAACATGGTTAAGGGTGAGAAAGGCCCCAAGGGCAAGA
AGATCACCCTCAAGGTGGCCAGGAATTGCATCAAAATCACTTTTGATGGGAAAAAGCGCCTTGACTTGAG
CAAGATGGGAATTACCACCTTCCCCAAGTGTATTCTGCGCCTTAGTGACATGGACGAGCTGGACCTTAGC
CGGAATCTTATCAGGAAGATCCCTGACTCCATCTCCAAGTTCCAGAACCTCCGGTGGCTGGACCTGCACA
GCAACTACATAGACAAGCTGCCTGAGTCCATTGGCCAGATGACCAGCCTGCTCTACCTCAACGTCAGCAA
CAACCGGCTGACCAGCAACGGGCTGCCCGTGGAGCTGAAGCAACTCAAGAACATCCGCGCTGTGAACCTA
GGCTTGAACCACCTGGACAGCGTGCCCACCACACTGGGGGCCCTGAAGGAGCTCCACGAGGTAGGGCTCC
ATGACAACCTACTGAACAACATCCCCGTGAGCATCTCCAAGCTCCCCAAGCTGAAAAAGCTCAACATAAA
GCGGAACCCCTTTCCAAAGCCAGGTGAGTCGGAAATATTCATAGACTCCATCAGGAGGCTGGAGAACTTG
TATGTTGTGGAGGAGAAGGATCTGTGTGCGGCTTGCCTGAGAAAATGCCAAAACGCCCGGGACAACCTGA
ATAGAATCAAGAACATGGCCACGACGACACCGAGAAAGACCATCTTTCCCAATCTGATCTCACCCAATTC
CATGGCCAAGGACTCCTGGGAAGACTGGAGGTGACTTGGAACCTGACCCTGAGGCAGAAGGGAAAGAGAG
AGGGAGGGAAGAGGGCA

In a search of public sequence databases, the NOV44a nucleic acid sequence, located on chromsome 10 has 188 of 313 bases (60%) identical to a gb:GENBANK-ID:AB016816|acc:AB016816.1 mRNA from Homo sapiens (Homo sapiens MASL1 mRNA, complete cds). Public nucleotide databases include all GenBank databases and the GeneSeq patent database.

The disclosed NOV44a polypeptide (SEQ ID NO:102) encoded by SEQ ID NO:101 has 255 amino acid residues and is presented in Table 44B using the one-letter amino acid code. Signal P, Psort and/or Hydropathy results predict that NOV44a has no signal peptide and is likely to be localized cytoplasmically with a certainty of 0.4500.

TABLE 44B
Encoded NOV44a protein sequence.

| | |
|---|---|
| MVKGEKVPKGKKITLKVARNCIKITFDGKKRLDLSKMGITTFPKCILRLSDMDELDLSRN | (SEQ ID NO:102) |
| LIRKIPDSISKFQNLRWLDLHSNYIDKLPESIGQMTSLLYLNVSNNRLTSNGLPVELKQL | |
| KNIRAVNLGLNHLDSVPTTLGALKELHEVGLHDNLLNNIPVSISKLPKLKKLNIKRNPFP | |
| KPGESEIFIDSIRRLENLYVVEEKDLCAACLRKCQNARDNLNRIKNMATTTPRKTIFPNL | |
| ISPNSMAKDSWEDWR | |

A search of sequence databases reveals that the NOV44a amino acid sequence has 211 of 255 amino acid residues (82%) identical to, and 235 of 255 amino acid residues (92%) similar to, the 262 amino acid residue ptnr:TREMBLNEW-ACC:BA1329635 protein from Mus musculus (Mouse) (ADULT MALE TESTIS CDNA, RIKEN FULL-LENGTH ENRICHED LIBRARY, CLONE:4921523N16, FULL INSERT SEQUENCE). Public amino acid databases include the GenBank databases, SwissProt, PDB and PIR.

NOV44a is expressed in at least cervix, brain, and testis. This information was derived by determining the tissue sources of the sequences that were included in the invention including but not limited to SeqCalling sources, Public EST sources, Literature sources, and/or RACE sources.

NOV44b

A disclosed NOV44b nucleic acid of 847 nucleotides (also referred to as CG57623-01) encoding a Leucine rich repeat-like protein is shown in Table 44C. The start and stop codons are in bold letters.

TABLE 44C
NOV44b nucleotide sequence.

| | |
|---|---|
| CTTCTAACACTCCCTTACTAAAAGAACATGGTTAAGGGTGAGAAAGGCCCCAAGGGCAAG | (SEQ ID NO:103) |
| AAGATCACCCTCAAGGTGGCCAGGAATTGCATCAAAATCACTTTTGATGGGAAAAAGCGC | |
| CTTGACTTGAGCAAGATGGGAATTACCACCTTCCCCAAGTGTATTCTGCGCCTTAGTGAC | |
| ATGGACGAGCTGGACCTTAGCCGGAATCTTATCAGGAAGATCCCTGACTCCATCTCCAAG | |
| TTCCAGAACCTCCGGTGGCTGGACCTGCACAGCAACTACATAGACAAGCTGCCTGAGTCC | |
| ATTGGCCAGATGACCAGCCTGCTCTACCTCAACGTCAGCAACAACCGGCTGACCAGCAAC | |
| GGGCTGCCCGTGGAGCTGAAGCAACTCAAGAACATCCGCGCTGTGAACCTAGGCTTGAAC | |
| CACCTGGACAGCGTGCCCACCATGCTGGGGGCCCTGAAGGAGCTCCACGAGGTAGGGCTC | |
| CATGACAACCTACTGAACAACATCCCCGTGAGCATCTCCAAGCTCCCCAAGCTGAAAAAG | |
| CTCAACATAAAGCGGAACCCCTTTCCAAAGCCAGGTGAGTCGGAAATATTCATAGACTCC | |
| ATCAGGAGGCTGGAGAACTTGTATGTTGTGGAGGAGAAGGATCTGTGTGCGGCTTGCCTG | |
| AGAAAATGCCAAAACGCCCGGGACAACCTGAATAGAATCAAGAACATGGCCACGACGACA | |
| CCGAGAAAGACCATCTTTCCCAATCTGATCTCACCCAATTCCATGGCCAAGGACTCCTGG | |
| GAAGACTGGAGGTGACTTGGAACCTGAGCCCTGAGGCAGAAGGGAAAGAGAGAGGGAGGG | |
| AAGAGGG | |

In a search of public sequence databases, the NOV nucleic acid sequence, located on chromsome 10 has 187 of 313 bases (59%) identical to a gb:GENBANK-ID:AB016816|acc:AB016816.1 mRNA from Homo sapiens (Homo sapiens MASL1 mRNA, complete cds). Public nucleotide databases include all GenBank databases and the GeneSeq patent database.

The disclosed NOV44b polypeptide (SEQ ID NO:104) encoded by SEQ ID NO:103 has 255 amino acid residues and is presented in Table B using the one-letter amino acid code. Signal P, Psort and/or Hydropathy results predict that NOV44b as no signal peptide and is likely to be localized cytoplasmically with a certainty of 0.4500.

TABLE 44D
Encoded NOV44b protein sequence.

| | |
|---|---|
| MVKGEKGPKGKKITLKVARNCIKITFDGKKRLDLSKMGITTFPKCILRLSDMDELDLSRN | (SEQ ID NO:104) |
| LIRKIPDSISKFQNLRWLDLHSNYIDKLPESIGQMTSLLYLNVSNNRLTSNGLPVELKQL | |
| KNIRAVNLGLNHLDSVPTMLGALKELHEVGLHDNLLNNIPVSISKLPKLKKLNIKRNPFP | |
| KPGESEIFIDSIRRLENLYVVEEKDLCAACLRKCQNARDNLNRIKNMATTTPRKTIFPNL | |
| ISPNSMAKDSWEDWR | |

A search of sequence databases reveals that the NOV44b amino acid sequence has 211 of 255 amino acid residues (82%) identical to, and 235 of 255 amino acid residues (92%) similar to, the 262 amino acid residue ptnr:SPTREMBL-ACC:Q9CQ07 protein from Mus musculus (Mouse) (4930442L21RIK PROTEIN). Public amino acid databases include the GenBank databases, SwissProt, PDB and PIR.

NOV44b is expressed in at least cervix, brain and testis. This information was derived by determining the tissue sources of the sequences that were included in the invention including but not limited to SeqCalling sources, Public EST sources, Literature sources, and/or RACE sources.

The disclosed NOV44 polypeptide has homology to the amino acid sequences shown in the BLASTP data listed in Table 44E.

TABLE 44E
BLAST results for NOV44

| Gene Index/ | | Length | Identity | Positives | |
|---|---|---|---|---|---|
| Identifier | Protein/Organism | (aa) | (%) | (%) | Expect |
| gi\|13385762\|ref\|NP_ | RIKEN cDNA | 262 | 211/255 | 235/255 | e−108 |
| 080529.1\| | 4930442L21 [Mus | | (82%) | (91%) | |
| (NM_026253) | musculus] | | | | |
| gi\|12838968\|dbj\|BAB24391.1\| | Leucine Rich | 255 | 210/255 | 234/255 | e−108 |
| (AK006063) | Repeat containing | | (82%) | (91%) | |
| | protein-data | | | | |
| | source: Pfam, | | | | |
| | source | | | | |
| | key: PF00560, | | | | |
| | evidence: ISS˜putative | | | | |
| | [Mus | | | | |
| | musculus] | | | | |
| gi\|12838360\|dbj\|BAB24176.1\| | Leucine Rich | 230 | 195/228 | 214/228 | e−97 |
| (AK005666) | Repeat containing | | (85%) | (93%) | |
| | protein-data | | | | |
| | source: Pfam, | | | | |
| | source | | | | |
| | key: PF00560, | | | | |
| | evidence: ISS˜putative | | | | |
| | [Mus | | | | |
| | musculus] | | | | |
| gi\|10130019\|gb\| | PIDD [Homo | 910 | 48/146 | 87/146 | 2e−18 |
| AAG13461.1\|AF274972_1 | sapiens ] | | (32%) | (58%), | |
| (AF274972) | | | | | |
| gi\|12083587\|ref\|NP_ | p53 protein | 915 | 47/146 | 87/146 | 2e−18 |
| 073145.1\| | induced, with | | (32%) | (59%) | |
| (NM_022654) | death domain [Mus | | | | |
| | musculus] | | | | |

Leucine-rich repeats (LRRs) are relatively short motifs (22-28 residues in length) found in a variety of cytoplasmic, membrane and extracellular proteins. Although these proteins are associated with widely different functions, a common property involves protein-protein interaction. Little is known about the 3D structure of LRRs, although it is believed that they can form amphipathic structures with hydrophobic surfaces capable of interacting with membranes. In vitro studies of a synthetic LRR from Drosophila Toll protein have indicated that the peptides form gels by adopting beta-sheet structures that form extended filaments. These results are consistent with the idea that LRRs mediate protein-protein interactions and cellular adhesion. Other functions of LRR-containing proteins include, for example, binding to enzymes and vascular repair. The 3-D structure of ribonuclease inhibitor, a protein containing LRRs, has been determined, revealing LRRs to be a new class of alpha/beta fold. LRRs form elongated non-globular structures and are often flanked by cysteine rich domains.

The disclosed NOV44 nucleic acid of the invention encoding a leucine-rich repeat-like protein includes the nucleic acid whose sequence is provided in Table 44A or a fragment thereof. The invention also includes a mutant or variant nucleic acid any of whose bases may be changed from the corresponding base shown in Table 44A while still encoding a protein that maintains its leucine-rich repeat-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 41 percent of the bases may be so changed.

The disclosed NOV44 protein of the invention includes the leucine-rich repeat-like protein whose sequence is provided in Table 44B. The invention also includes a mutant or variant protein any of whose residues may be changed from the corresponding residue shown in Table B while still encoding a protein that maintains its leucine-rich repeat-like activities and physiological functions, or a functional fragment thereof. In the mutant or variant protein, up to about 18 percent of the residues may be so changed.

The invention further encompasses antibodies and antibody fragments, such as F_{ab} or (F_{ab})₂, that bind immunospecifically to any of the proteins of the invention.

The above defined information for this invention suggests that this leucine-rich repeat-like protein (NOV44) may function as a member of a “leucine-rich repeat family”. Therefore, the NOV44 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV44 nucleic acids and proteins of the invention are useful in potential therapeutic applications implicated in diseases including but not limited to various pathologies and disorders as indicated below. For example, a cDNA encoding the leucine-rich repeat-like protein (NOV44) may be useful in gene therapy, and the leucine-rich repeat-like protein (NOV44) may be useful when administered to a subject in need thereof. By way of nonlimiting example, the compositions of the present invention will have efficacy for treatment of patients suffering from fertility, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, or other pathologies or conditions. The NOV44 nucleic acid encoding the leucine-rich repeat-like protein of the invention, or fragments thereof, may further be useful in diagnostic applications, wherein the presence or amount of the nucleic acid or the protein are to be assessed.

NOV44 nucleic acids and polypeptides are further useful in the generation of antibodies that bind immuno-specifically to the novel NOV44 substances for use in therapeutic or diagnostic methods. These antibodies may be generated according to methods known in the art, using prediction from hydrophobicity charts, as described in the “Anti-NOVX Antibodies” section below. The disclosed NOV44 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV45

NOV45 includes two Ig/fibronectin-like proteins disclosed below. The disclosed sequences have been named NOV45a and NOV45b.

NOV45a

A disclosed NOV45a nucleic acid of 4321 nucleotides (also referred to as CG57656-01) encoding a Ig/fibronectin-like protein is shown in Table 45A. The start and stop codons are in bold letters.

TABLE 45A
NOV45a nucleotide sequence.

(SEQ ID NO:105)

CTGGTGGGGGGCGGGGTGACCCTGTGACACGGACATGGGGCTGCTGGGGCAGGATCTCTTTGTCACCTCC
TTTCTGTGTCCAACCTGGCCGTCCCCATCAGGCGCCCACGGCCTGCGAGAGGAGCCCGAGTTTGTGACGG
CAAGAGCTGGGCAGAGCGTGGTCCTGCGATGCGACGTGATCCACCCAGTGACGGGACAGCCCCCACCCTA
TGTCGTAGAGTGGTTCAAGTTCGGGGTCCCCATCCCTATCTTCATCAAGTTTGGCTACTACCCGCCGCAC
GTGGACCCTGAGTATGCAGGTAAGGTCGGCCCCCACGGCCTGCGAGAGGAGCCCGAGTTTGTGACGGCAA
GAGCTGGGGAGAGCGTGGTCCTGCGATGCGACGTGATCCACCCAGTGACGGGACAGCCCCCACCCTATGT
CGTAGAGTGGTTCAAGTTCGGGGTCCCCATCCCTATCTTCATCAAGTTTGGCTACTACCCGCCGCACGTG
GACCCTGAGTATGCAGGTAAGGTCAGTCTTCATGATAAGGCATCTCTGCGGCTGGAACAAGTTCGCTCTG
AGGACCAGGGCTGGTATGAGTGCAAAGTGCTCATGCTGGACCAGCAGTATGACACCTTCCACAATGGCAG
CTGGGTCCACCTCACCATCAACGCCCCTCCCACCTTTACAGAAACACCCCCCCAGTACATCGAGGCCAAG
GAGGGTGGTAGTATCACCATGACCTGCACAGCTTTTGGGAACCCCAAGCCCATTGTCACCTGGCTCAAGG
AGGGGACGCTCCTCGGTGCTAGTGGGAAATACCAGGTGAGTGTGGTTCTAGGTAGCCTGACAGTGACATC
GGTCAGTCGGGAGGACAGAGGTGCCTACACCTGCCGAGCGTACAGCATTCAGGGGGAGGCTGTCCACACG
ACTCACCTGCTTGTCCAAGGGCCCCCTTTCATCGTCTCCCCTCCTGAGAACATCACCGTCAACATCTCCC
AGGATGCTCTGCTCACCTGCCGGGCAGAGGCGTATCCGGGCAACCTCACCTACACCTGGTACTGGCAGGA
CGAGAACGTCTACTTTCAGAACGACCTGAAGCTGAGGGTGCGCATCCTAATCGATGGGACCCTGATCATC
TTCCGGGTGAAGCCGGAGGACTCAGGGAAGTACACCTGTGTGCCCAGCAACAGCCTGGGGCGCTCCCCCT
CCGCCTCGGCGTACCTGACCGTGCAGTACCCAGCGCGTGTCCTCAACATGCCCCCTGTGATTTACGTGCC
CGTGGGGATCCATGGCTACATCCGCTGCCCTGTGGACGCAGAACCACCGGCCACCGTGGTCAAGTGGAAC
AAGGACGGCCGTCCCCTGCAGGTTGAGAAGAACCTCGGTTGGACCCTGATGGAGGATGGCTCCATTCGAA
TTGAGGAGGCCACAGAGGAGGCTCTTGGCACTTATACCTGTGTGCCTTACAACACTCTGGGGACCATGGG
CCAGTCTGCCCCTGCGAGGCTTGTCCTGAAGGACCCCCCCTATTTCACGGTGCTACCAGGCTGGGAGTAC
AGGCAGGAGGCCGGCCGGGAGCTACTTATCCCCTGTGCTGCCGCAGGGGACCCCTTTCCTGTCATCACTT
GGAGAAAGGTAGGGAAGCCCAGCAGAAGCAAGCACAGTGCCCTGCCCAGTGGGAGCCTGCAGTTCCGTGC
CCTGAGTAAGGAGGACCACGGGGAGTGGGAATGTGTCGCCACCAACGTGGTCACGAGCATCACTGCCAGC
ACCCACCTCACCGTCATCGGTACGGGCACCAGCCCCCATGCCCCGGGCAGTGTCCGGGTCCAGGTCTCCA
TGACAACTGCCAACGTGTCCTGGGAACCAGGCTATGATGGAGGCTACGAGCAGACATTCTCAGTTTGGTA
CGGACCTCTGATGAAGCGGGCACAGTTTGGGCCCCATGACTGGCTGTCCTTGCCAGTGCCGCCAGGACCC
AGCTGGCTGCTCGTGGACACCCTGGAGCCTGAGACAGCGTACCAGTTCAGCGTCCTGGCCCAGAAGCTGG
GAACCAGCGCCTTCAGTGAGGTGGTCACTGTGAACACTTTAGCATTCCCTATTACAACTCCAGAACCCCT
GGTGCTGGTCACCCCACCGAGGTGCCTCATAGCCAATCGGACTCAGCAGGGTGTGCTCCTGTCCTGGCTT
CCGCCTGCCAACCACAGCTTTCCCATCGACCGCTACATCATGGAGTTCCGTGTCGCAGAGCGCTGGGAGT
TGCTCGACGATGGCATCCCCGGCACCGAAGGAGAGTTCTTTGCCAAGGATCTGTCACAGGACACGTGGTA
TGAGTTCCGGCTTCTGGCCGTCATGCAGGATCTGATCGGCGAGCCCAGCAACATCGCCGGCGTCTCCAGC
ACAGACATCTTCCCGCAGCCGGACCTGACCGAGGATGGGCTGGCGCGGCCTGTGCTGGCGGGAATCGTAC
CTACCATCTGCTTCTTGGCAGCTGCCATCCTGTTCAGCACCCTGGCTGCCTGCTTTGTCAACAAGCAGCG
CAAGCGTAAGCTCAAGCGCAAAAAAGACCCTCCACTCTCCATCACCCACTGCAGGAAGAGCCTGGAGTCT
CCCTTGTCCTCTGGCAAGGTGAGCCCCGAGAGCATCCGCACGCTCCGAGCGCCGTCAGAATCCTCCGACG
ACCAGGGCCAGCCCGCGGCCAAGAGGATGCTGAGCCCCACCCGTGAGAAGGAGCTGTCGCTGTACAAGAA
GACCAAGCGGGCCATCAGCAGCAAGAAGTACAGCGTGGCCAAGGCAGAGGCCGAGGCAGAGGCCACCACG
CCCATCGAGCTCATCAGCAGAGGCCCTGACGGCCGCTTCGTGATGGACCCTGTCGAGATGGAGCCCTCGC
TGAAGAGCAGGCGCATCGAGGGCTTCCCCTTCGCCGAGGAGACGGACATGTACCCCGAGTTCCGCCAGTC
GGACGAGGAGAACGAGGACCCACTGGTGCCCACATCTGTGGCCGCCCTGAAGTCCCAGCTCACCCCTCTG
TCATCCAGCCAGGAGTCCTACCTGCCACCACCAGCATACAGCCCTCGGTTCCAGCCCCGCGGGCTGGAGG
GCCCCGGTGGCCTGGAAGGTCGGCTTCAGGCCACAGGCCAGGCCCGGCCCCCTGCCCCCCGGCCCTTCCA
CCATGGCCAGTATTATGGGTACCTCAGCAGCAGCAGCCCTGGGGAGGTGGAGCCGCCCCCGTTCTACGTG
CCAGAAGTGGGCAGCCCCCTGAGCTCCGTCATGTCGTCCCCGCCCCTGCCCACCGAGGGGCCCTTTGGCC
ACCCCACCATCCCCGAGGAGAATGGAGAGAATGCATCCAACAGCACGCTGCCCTTGACTCAGACACCTAC
AGGAGGGCGCTCCCCTGAGCCCTGGGGCCGGCCAGAATTCCCCTTCGGGGGGCTGGAGACCCCAGCGATC
ATGTTCCCCCACCAGCTGCCACCCTGTGATGTGCCCGAGAGTCTGCAGCCCAAGGCCGGCCTCCCCCGAG
GACTGCCCCCCACCTCCCTGCAGGTGCCCGCGGCCTACCCGGGCATCCTGTCTCTGGAGGCACCGAAGGG
TTGGGCAGGCAAGTCGCCCGGCAGGGGCCCTGTCCCAGCGCCCCCCGCCGCCAAGTGGCAGGACAGACCT
ATGCAACCTCTGGTAAGCCAAGGGCAGCTGCGACATACAAGCCAAGGCATGGGCATACCTGTGCTGCCTT
ACCCCGAGCCGGCTGAGCCGGGGGCGCACGGCGGCCCCAGCACATTTGGCCTGGACACCCGGTGGTATGA
GCCCCAGCCCCGGCCCCGGCCTAGCCCTCGGCAGGCCAGGCGCGCCGAGCCCAGTTTACATCAAGTGGTG
CTACAGCCCTCCCGGCTCTCACCTCTGACCCAAAGCCCCCTCAGCTCCCGCACCGGCTCCCCTGAGCTCG
CCGCCCGTGCCCGGCCTCGCCCGGGCCTCCTGCAGCAGGCAGAGATGTCAGAGATCACCCTGCAGCCGCC
GGCTGCAGTCAGCTTTTCTCGAAAGTCTACGCCGTCCACAGGCTCCCCCTCCCAGAGCAGCCGCAGTGGG
AGTCCCAGCTACCGGCCCGCCATGGGCTTCACCACTCTGGCCACCGGCTACCCTTCCCCTCCACCCGGCC
CCGCCCCTGCTGGGCCTGGGGACAGCTTGGACGTGTTTGGACAGACGCCTTCCCCTCGAAGGACGGGGGA
GGAATTGCTCCGACCGGAGACCCCACCACCCACGTTACCTACTTCAGGGAAGCTGCGGAGAGACAGACCA
GCTCCCGCGACCAGCCCGCCTGAGACAGCACTCTCTAAACTGTAGCAGCTG

In a search of public sequence databases, the NOV45a nucleic acid sequence, located on chromsome II has 2296 of 2298 bases (99%) identical to a gb:GENBANK-ID:AB028953|acc:AB028953.1 mRNA from Homo sapiens (Homo sapiens mRNA for KIAA1030 protein, partial cds). Public nucleotide databases include all GenBank databases and the GeneSeq patent database.

The disclosed NOV45a polypeptide (SEQ ID NO:106) encoded by SEQ ID NO:105 has 1328 amino acid residues and is presented in Table 45B using the one-letter amino acid code. Signal P, Psort and/or Hydropathy results predict that NOV45a has a signal peptide and is likely to be localized at the plasma membrane with a certainty of 0.4600. The most likely cleavage site for a NOV45a peptide is between amino acids 19 and 20.

TABLE 45B
Encoded NOV45a protein sequence.

| | |
|---|---|
| MGLLGQDLFVTSFLCPTWPSPSGAHGLREEPEFVTARAGESVVLRCDVIHPVTGQPPPYV | (SEQ ID NO:106) |
| VEWFKFCVPIPIFIKFGYYPPHVDPEYAGKVGAHGLREEPEFVTARAGESVVLRCDVIHP | |
| VTGQPPPYVVEWFKFGVPIPIFIKFGYYPPHVDPEYAGKVSLHDKASLRLEQVRSEDQGW | |
| YECKVLMLDQQYDTFHNGSWVHLTINAPPTFTETPPQYIEAKEGGSITMTCTAFGNPKPI | |
| VTWLKEGTLLGASGKYQVSVVLGSLTVTSVSREDRGAYTCRAYSIQGEAVHTTHLLVQGP | |
| PFIVSPPENITVNISQDALLTCRAEAYPGNLTYTWYWQDENVYFQNDLKLRVRILIDGTL | |
| IIFRVKPEDSGKYTCVPSNSLGRSPSASAYLTVQYPARVLNMPPVIYVPVGIHGYIRCPV | |
| DAEPPATVVKWNKDGRPLQVEKNLGWTLMEDGSIRIEEATEEALGTYTCVPYNTLGTMGQ | |
| SAPARLVLKDPPYFTVLPGWEYRQEAGRELLIPCAAAGDPFPVITWRKVGKPSRSKHSAL | |
| PSGSLQFRALSKEDHGEWECVATNVVTSITASTHLTVIGTGTSPHAPGSVRVQVSMTTAN | |
| VSWEPGYDGGYEQTFSVWYGPLMKRAQFGPHDWLSLPVPPGPSWLLVDTLEPETAYQFSV | |
| LAQKLGTSAFSEVVTVNTLAFPITTPEPLVLVTPPRCLIANRTQQGVLLSWLPPANHSFP | |
| IDRYIMEFRVAERWELLDDGIPGTEGEFFAKDLSQDTWYEFRVLAVMQDLIGEPSNIAGV | |
| SSTDIFPQPDLTEDGLARPVLAGIVATICFLAAAILFSTLAACFVNKQRKRKLKRKKDPP | |
| LSITHCRKSLESPLSSGKVSPESIRTLRAPSESSDDQGQPAAKRMLSPTREKELSLYKKT | |
| KRAISSKKYSVAKAEAEAEATTPIELISRGPDGRFVMDPVEMEPSLKSRRIEGFPFAEET | |
| DMYPEFRQSDEENEDPLVPTSVAALKSQLTPLSSSQESYLPPPAYSPRFQPRGLEGPGGL | |
| EGRLQATGQARPPAPRPFHHGQYYGYLSSSSPGEVEPPPFYVPEVGSPLSSVMSSPPLPT | |
| EGPFGHPTIPEENGENASNSTLPLTQTPTGGRSPEPWGRPEFPFGGLETPAMMFPHQLPP | |
| CDVPESLQPKAGLPRGLPPTSLQVPAAYPGILSLEAPKGWAGKSPGRGPVPAPPAAKWQD | |
| RPMQPLVSQGQLRHTSQGMGIPVLPYPEPAEPGAHGGPSTFGLDTRWYEPQPRPRPSPRQ | |
| ARRAEPSLHQVVLQPSRLSPLTQSPLSSRTGSPELAARARPRPGLLQQAEMSEITLQPPA | |
| AVSFSRKSTPSTGSPSQSSRSGSPSYRPAMGFTTLATGYPSPPPGPAPAGPGDSLDVFGQ | |
| TPSPRRTGEELLRPETPPPTLPTSGKLRRDRPAPATSPPERALSKL | |

A search of sequence databases reveals that the NOV45a amino acid sequence has 761 of 763 amino acid residues (99%) identical to, and 761 of 763 amino acid residues (99%) similar to, the 763 amino acid residue ptnr:SPTREMBL-ACC:Q9UPX0 protein from Homo sapiens (Human) (KIAA1030 PROTEIN). Public amino acid databases include the GenBank databases, SwissProt, PDB and PIR.

NOV45a is expressed in at least brain, cerebral medulla/cerebral white matter, prostate, thalamus, placenta. Expression information was derived from the tissue sources of the sequences that were included in the derivation of the sequence of CG57656-01. The sequence is predicted to be expressed in the following tissues because of the expression pattern of (GENBANK-ID: gb:GENBANK-ID:AB028953|acc:AB028953.1) a closely related Homo sapiens mRNA for KIAA1030 protein, partial cds homolog in species Homo sapiens liver. This information was derived by determining the tissue sources of the sequences that were included in the invention including but not limited to SeqCalling sources, Public EST sources, Literature sources, and/or RACE sources.

NOV45b

A disclosed NOV45b nucleic acid of 7097 nucleotides (also referred to as CG57656-02) encoding a Ig/fibronectin-like protein is shown in Table 45C. The start and stop codons are in bold letters.

TABLE 45C
NOV45b nucleotide sequence.

| | |
|---|---|
| ATGGGGCTGCTGGGGCAGGATCTCTTTGTCACCTCCTTTCTGTGTCCAACCTGGCCGTCC | (SEQ ID NO:107) |
| CCATCAGGCGCCCACGGCCTGCGAGAGGAACCCGAGTTTGTGACGGCAAGAGCTGGGGAG | |
| AGCGTGGTCCTGCGATGCGACGTGATCCACCCAGTGACGGGACAGCCCCCACCCTATGTC | |
| GTAGAGTGGTTCAAGTTCGGGGTCCCCATCCCTATCTTCATCAAGTTTGGCTACTACCCC | |
| CCACACGTGGACCCTGAGTATGCAGGCCGGGCCAGTCTTCATGATAAGGCATCTCTGCGG | |
| CTGGAACAAGTTCGCTCTGAGGACCTGGGCTGGTATGAGTGCAAAGTGCTCATGCTGGAC | |
| CAGCAGTATGACACCTTCCACAATGGCAGCTGGGTCCACCTCACCATCAACGCCCCTCCC | |
| ACCTTTACAGAAACACCCCCCCGGTACATCGAGGCCAAGGAGGGTGGTAGTATCACCATG | |
| ACCTGCACAGCTTTTGGGAACCCCAAGCCCATTGTCACCTGGCTCAAGGAGGGGACGCTC | |
| CTCGGTGCTAGTGGGAAATACCAGGTGAGTGACGGCAGCCTGACAGTGACATCGGTCAGT | |
| CGGGAGGACAGAGGTGCCTACACCTGCCGAGCGTACAGCATTCAGGGGGAGGCTGTCCAC | |
| ACGACTCACCTGCTTGTCCCAGGGCCCCCTTTCATCGTCTCCCCTCCTGAGAACATCACC | |
| GTCAACATCTCCCAGGATGCTCTGCTCACCTGCCGGGCAGAGGCGTATCCGGGCAACCTC | |
| ACCTACACCTGGTACTGGCAGGACGAGAACGTCTACTTTCAGAACGACCTGAAGCTGAGG | |
| GTGCGCATCCTAATCGATGGGACCCTGATCATCTTCCGGGTGAAGCCGGAGGACTCGGGG | |
| AAGTACACCTGTGTGCCCAGCAACAGCCTGGGGCGCTCCCCCTCCGCCTCGGCGTACCTG | |
| ACCGTGCAGTACCCAGCGCGTGTCCTCAACATGCCCCCTGTGATTTACGTGCCCGTGGGG | |
| ATCCATGGCTACATCCGCTGCCCTGTGGACGCAAGACCACCGGCCACCGTGGTCAAGTGG | |
| AACAAGGACGGCCGTCCCCTGCAGGTTGAGAAGAACCGCGGTTGGACCCTGATGGAGGAT | |
| GGCTCCATTCGAATTGAGGAGGCCACAGAGGAGGCTCTTGGCACTTATACCTGTGTGCCT | |
| TACAACACTCTGGGGACCATGGGCCAGTCTGCCCCTGCGAGGCTTGTCCTGAAGGACCCC | |
| CCCTATTTCACGGTGCTACCAGGCTGGGAGTACAGGCAGGAGGCCGGCCGGGAGCTACTT | |
| ATCCCCTGTGCTGCCGCAGGGGACCCCTTTCCTGTCATCACTTGGAGCAAGGTAGGGAAG | |
| CCCAGCAGAAGCAAGCACAGTGCCCTGCCCAGTGGGAGCCTGCAGTTCCGTGCCCTGAGT | |
| AAGGAGGACCACGGGGAGTGGGAATGTGTCGCCACCAACGTGGTCACGAGCATCACTGCC | |
| AGCACCCACCTCACCGTCATCGGCACCAGCCCCCATGCCCCGGGCAGTGTCCGGGTCCAG | |
| GTCTCCATGACAACTGCCAACGTGTCCTGGGAACCAGGTGACGGGCTACGATGGGGCTAT | |
| GATGGAGGCTACGAGCAGACATTCTCAGTTTGGATGAAGCGGGCACAGTTTGGGCCCCAT | |
| GACTGGCTGTCCTTGCCAGTGCCGCCAGGACCCAGCTGGCTGCTGGTGGACACCCTGGAG | |
| CCTGAGACAGCGTACCAGTTCAGCGTCCTGGCCCAGAACAAGCTGGGAACCAGCGCCTTC | |
| AGTGAGGTGGTCACTGTGATCACTTTAGCATTCCCTATTACAACTCCAGAACCCCTGGTG | |
| CTGGTCACCCCACCGAGGTGCCTCATAGCCAATCGGACTCAGCAGGGTGTGCTCCTGTCC | |
| TGGCTTCCGCCTGCCAACCACAGCTTTCCCATCGACCGCTACATCATGGAGTTCCGTGTC | |
| GCAGAGCGCTGGGAGTTGCTCGACGATGGCATCCCCGGCACCGAAGGAGAGTTCTTTGCC | |
| AAGGATCTGTCACAGGACACGTGGTATGAGTTCCGGGTTCTGGCCGTCATGCAGGATCTG | |
| ATCGGCGAGCCCAGCAACATCGCCGGCGTCTCCAGCACAGACATCTTCCCGCAGCCGGAC | |
| CTGACCGAGGATGGGCTGGCGCGGCCTGTGCTGGCGGGAATCGTAGCTACCATCTGCTTC | |
| TTGGCAGCTGCCATCCTGTTCAGCACCCTGGCTGCCTGCTTTGTCAACAAGCAGCGCAAG | |
| CGTAAGCTCAAGCGCAAAAAAGACCCTCCACTCTCCATCACCCACTGCAGGAAGAGCCTG | |
| GAGTCTCCCTTGTCCTCTGGCAAGGTGAGCCCCGAGAGCATCCGCACGCTCCGAGCGCCG | |
| TCAGAATCCTCCGACGACCAGGGCCAGCCCGCGGCCAAGAGGATGCTGAGCCCCACCCGT | |
| GAGAAGGAGCTGTCGCTGTACAAGAAGACCAAGCGGGCCATCAGCAGCAAGAAGTACAGC | |
| GTGGCCAAGGCAGAGGCCGAGGCAGAGGCCACCACGCCCATCGAGCTCATCAGCAGAGGC | |
| CCTGACGGCCGCTTCGTGATGGACCCTGTCGAGATGGAGCCCTCGCTGAAGAGCAGGCGC | |
| ATCGAGGGCTTCCCCTTCGCCGAGGAGACGGACATGTACCCCGAGTTCCGCCAGTCGGAC | |
| GAGGAGAACGAGGACCCACTGGTGCCCACATCTGTGGCCGCCCTGAAGTCCCAGCTCACC | |
| CCTCTGTCATCCAGCCAGGAGTCCTACCTGCCACCACCAGCATACAGCCCTCGGTTCCAG | |
| CCCCGCGGGCTGGAGGGCCCCGGTGGCCTGGAAGGTCGGCTTCAGGCCACAGGCCAGGCC | |
| CGGCCCCCTGCCCCCCGGCCCTTCCACCATGGCCAGTATTATGGGTACCTCAGCAGCAGC | |
| AGCCCTGGGGAGGTGGAGCCGCCCCCGTTCTACGTGCCAGAAGTGGGCAGCCCCCTGAGC | |
| TCCGTCATGTCGTCCCCGCCCCTGCCCACCGAGGGGCCCTTTGGCCACCCCACCATCCCC | |
| GAGGAGAATGGAGAGAATGCATCCAACAGCACGCTGCCCTTGACTCAGACACCTACAGGA | |
| GGGCGCTCCCCTGAGCCCTGGGGCCGGCCAGAATTCCCCTTCGGGGGGCTGGAGACCCCA | |
| GCGATGATGTTCCCCCACCAGCTGCCACCCTGTGATGTGCCCGAGAGTCTGCAGCCCAAG | |
| GCCGGCCTCCCCCGAGGACTGCCCCCCACCTCCCTGCAGGTGCCCGCGGCCTACCCGGGC | |
| ATCCTGTCTCTGGAGGCACCGAAGGGTTGGGCAGGCAAGTCGCCCGGCAGGGGCCCTGTC | |
| CCAGCGCCCCCCGCCGCCAAGTGGCAGGACAGACCTATGCAACCTCTGGTAAGCCAAGGG | |
| CAGCTGCGACATACAAGCCAAGGCATGGGCATACCTGTGCTGCCTTACCCCGAGCCGGCT | |
| GAGCCGGGGGCGCACGGCGGCCCCAGCACATTTGGCCTGGACACCCGGTGGTATGAGCCC | |
| CAGCCCCGGCCCCGGCCTAGCCCTCGGCAGGCCAGGCGCGCCGAGCCCAGTTTACATCAA | |
| GTGGTGCTACAGCCCTCCCGGCTCTCACCTCTGACCCAAAGCCCCCTCAGCTCCCGCACC | |
| GGCTCCCCTGAGCTCGCCGCCCGTGCCCGGCCTCGCCCGGGCCTCCTGCAGCAGGCAGAG | |
| ATGTCAGAGATCACCCTGCAGCCGCCGGCTGCAGTCAGCTTTTCTCGAAAGTCTACGCCG | |
| TCCACAGGCTCCCCCTCCCAGAGCAGCCGCAGTGGGAGTCCCAGCTACCGGCCCGCCATG | |
| GGCTTCACCACTCTGGCCACCGGCTACCCTTCCCCTCCACCCGGCCCCGCCCCTGCTGGG | |
| CCTGGGGACAGCTTGGACGTGTTTGGACAGACGCCTTCCCCTCGAAGGACGGGGGAGGAA | |
| TTGCTCCGACCGGAGACCCCACCACCCACGTTACCTACTTCAGGGAAGCTGCAGAGAGAC | |
| AGACCAGCTCCCGCGACCAGCCCGCCTGAGAGAGCACTCTCTAAACTGTAGCAGCTGGTA | |
| TTCCAGCTATCTGGGCAGTGTTGTCGAGACAAGCCTCTCCTCAGCTCAATAGGTAAGGGA | |
| GCTCCTCGGCTGGGCGGGCGGGGGCGGGCAGGCGGACGGGGCTTCGGCCGGGCCATTGCT | |
| TCCTGGACAGGGGATCCAAACCATGTCCCCTCACCGCCCCGTGGGGTGGCCGCTGCCGCT | |
| CCTCGATCCCCACGGCTTCTGGGTTCCCCACATCGAGCCACGCTCGGCACCGCCTAGCTG | |
| CAGCTTCTGCCCCCCACCCCCCGTCCCCATGTCCGGCCCCTCTAGAGCCCCCTGCGGCTC | |
| TCCTCACTCCTCGCCAGCCTCGCCAGCCTGCTGCCAGTACACCCAGGGCCTCCCACAGAA | |
| GCCCTGGGGCCCTGCATGCACCCCGAAGGGGCCCAGAACACCCGAGATCTGCTTTGCATC | |
| TCTGCACCCTTGGGGACCTCTCTGGGGCCCCTCTGGTATCCAGGAAGAGCCCCCCCGCAC | |
| CCTATTCTCCCAGCAGCCCCAGGCATAACATCCCTTCTCCTGGGGTGGCTGGCCCCTCAC | |
| CAGCTTCTTGAGATCTTCGTGGTATTAGGCTCTCTCGAGGAGTACAAGGTTGGGATGAGC | |
| CCCACTTCTTCCTTGACAGTGGGGTCCTCTGCAGGGTCAGGTCCCTGCAGTCCTGTGGGG | |
| CTTTGTAGCGAAATGTCATCACTCCCCTGTGCTCCTTGCCTTCTGCAGCCTGACCTTGGT | |
| AGTCACAGGACTGAAATGTGACCTAGCCCTTGGGTCCACATTGCTTTCAAGGTCCCTGTT | |
| GTAGCCTTGTCGTCCTCCCTGGGTCATGCTGTAGGGAAAGGGTCTTAGGAGGACCTCCCC | |
| GAGGGGAGGGGGCAGGCTTCCCCTGGGCAGACAGGCATTGAGCTGGCAGGAAGTGAAACC | |
| CCCAGGGACCAGCACTGTGTCCCTCCCCCTGCCCCCGCAGTCAGCCTCTCCTTGAGTGTC | |
| CTTCCAGGTGGAAGCTAAAGGAACTTGGGCATTCAGGGAGCCTCTGAGTCCCTCACAGAG | |
| ACTCAGGGCCACCCGAAGCTCGCTCCCTCTCACAGTAGCCTGACAAGGTGCTGCCGCTCG | |
| CCACGGACCCTCTGCCCTGTGCCTGGGCACACACAGGCATCGGGCACCTGCATGGGAGAC | |
| GGCGAGCCTCCCGTCCAGGTGCTTCTGGCTTTCCAGGCGAGAAGGAGACAGGTGCCTTCC | |
| CCCCCTAGAGATGTCAAGGGAGGTTCACTTTCCTAACCAGGGCTATAAATCTCATTCATT | |
| CCTAAGAGTGGCCTCCATAAAGAGGACTGCCCTGACATTTCTTTACCATCTGTAGCTATA | |
| AAATTGTCAGCAGCCCAGAGGCCCTGGAATTGTGGGTGAGGCTGTCTGGCATAGGGTGAC | |
| GTGAGGGCTGTCAGGCAGCGTTTATGAGCTCACCTGCTCTGGGGCCCTCCTGCGCCCAG | |
| AGACCCACTGGTCTTCCACCTTCCCCAGCTCCCTCCCTGTCCCAGCTGTGACTCCTCCAC | |
| AGCCCCCGGGAGGTGTGGGAGCCCTAGACTAGCTCTCACCCCAGCTCCTGGAAATTCCTA | |
| GACTCTGCTCTCATGTGTTATTCCTCACCCCTTCACTCAACTCCATTGACCCTCCCCTTT | |
| CCCAGTGTCCCCACTGTGCCAGATCCAAGAGAAGCCCGCTCTCCTTTCACTGCTGTGGCA | |
| AACCCAGAAACCAGGGGCAGCGATGAGGGACCATCGTTTCTCTCCCGGGCCTGGCCAGCA | |
| TCCCCAGCCTAGGAGAAGGAGACCCTCCCCATCCTGAGTCAGCCCCTCGGTGCTGGCCTC | |
| TCCTGCCTGCTGGGAGCCTCCCCGGTACGGCTGCTGGGTCCTGGGGAGATGCAGGCTCTG | |
| TTCAGATGCTGTCTCCATGCTGCACCTTTGCATGTGTGCCCTCTTGGTCTTGCTTGGAGA | |
| AGTTGTGACAGCTCTTGTCCACGTGCGTTCTCACTGTTTCTTTTCCCTTCAGTTCCTCTC | |
| CTGTTCCTTTGGTCCATATGTAGTTGTTGCCCCTGCCTCTCCTTTTCTCTCTCCCTTTTT | |
| TTCTCCTCTTCCCAACTCCCTTCTCAACATGGGGAGTTGTATTGCGTGGACCTGTCCTTT | |
| CTGTGGACTGTGGGGAGGCCGAGCCAATTCTTAGCTCCTGTCTGAGCCTAGTGATGCCCT | |
| GTCCTCACCTCTGCTTCCACTCCCTTTCCCATTCAGGCTGACTGTCACCGCAGGGAGGCA | |
| GGTGGGTCTGGAGCATGGAGTGGCCCCTGTCCTGGGTCAGCTGTAGGAAGGGGCCCCTTT | |
| GTAAACAAGCTTCTGCCAGGTTCTGAGGCAGAGCACTGGTCTGAGTCCGGCCCCACAGAA | |
| GGGTTCCATCTTGTGGTCAGCGGGGTGGGCTCAGACCTGCAGACTCCCGCAGTGGGCCCA | |
| CCCTTTCCCTTGCTATGGCCCGTCTATGAGTGGCTTTCCCCTGCCTCCATCGGTGTATAA | |
| TCAGGCAGGTGGTGTTTGCTCTTAGGTGATTAGAAGAGGAGGAAAATGCCCATCAAGCCA | |
| CTGCTTCAGCCTGTTGGCCAGGGAACACCTGAACAGGGTCAAAATGGTCAGCTCCTGGGC | |
| GCTGAGCTAAGGAAATATGCAGGGGGTTTTGACTTTCCCTGTCCTGAGGCTTGCCTCCAC | |
| TTTGACCTGGCTGCATCTTTCCTCAGCTCTTCTGGGTAAGCTGAGAGCTAAGCAAATAAG | |
| ACCTCCTGGGCTACCTGGTCATGATTTGGGTTTTGTATGGATTTTTTGAAAAGAGAGAAA | |
| AAGAGTGTGGGCTGGGTGCCGTGCCCCAGAGGGCAGATCTCTTGAGTCCAGGAGTTCAAG | |
| GCCAGCCTGGGCAACATAGTGAAGCCCCATCTCTGCAAAAAAATACAAAAAAATAGCTGG | |
| GCATGGTGGCACATGCCTATAGTCCCAGCTCCTTGGGGGGCTGAGGTGGGAAGATCATCT | |
| GAGCTGGGGAGGTCGAGGCAGCAGTGAGCCGAGATTGCGCCACTTCACTCCAGCCTGGGT | |
| GACAGAGATCCTGTCTC | |

In a search of public sequence databases, the NOV45b nucleic acid sequence, located on chromsome 11 has 5312 of 5319 bases (99%) identical to a gb:GENBANK-ID:AB028953|acc:AB028953.1 mRNA from Homo sapiens (Homo sapiens mRNA for KIAA1030 protein, partial cds). Public nucleotide databases include all GenBank databases and the GeneSeq patent database.

The disclosed NOV45b polypeptide (SEQ ID NO:108) encoded by SEQ ID NO:107 has 1356 amino acid residues and is presented in Table 45D using the one-letter amino acid code. Signal P, Psort and/or Hydropathy results predict that NOV45b has a signal peptide and is likely to be localized at the plasma membrane with a certainty of 0.4600. The most likely cleavage site for a NOV45b peptide is between amino acids 19 and 20.

TABLE 45D
Encoded NOV45b protein sequence.

| | |
|---|---|
| MGLLGQDLFVTSFLCPTWPSPSGAHGLREEPEFVTARAGESVVLRCDVIHPVTGQPPPYV | (SEQ ID NO:198) |
| VEWFKFGVPIPIFIKFGYYPPHVDPEYAGRASLHDKASLRLEQVRSEDLGWYECKVLMLD | |
| QQYDTFHNGSWVHLTINAPPTFTETPPRYIEAKEGGSITMTCTAFGNPKPIVTWLKEGTL | |
| LGASGKYQVSDGSLTVTSVSREDRGAYTCRAYSIQGEAVHTTHLLVPGPPFIVSPPENIT | |
| VNISQDALLTCRAEAYPGNLTYTWYWQDENVYFQNDLKLRVRILIDGTLIIFRVKPEDSG | |
| KYTCVPSNSLGRSPSASAYLTVQYPARVLNMPPVIYVPVGIHGYIRCPVDARPPATVVKW | |
| NKDGRPLQVEKNRGWTLMEDGSIRIEEATEEALGTYTCVPYNTLGTMGQSAPARLVLKDP | |
| PYFTVLPGWEYRQEAGRELLIPCAAAGDPFPVITWSKVGKPSRSKHSALPSGSLQFRALS | |
| KEDHGEWECVATNVVTSITASTHLTVIGTSPHAPGSVRVQVSMTTANVSWEPGDGLRWGY | |
| DGGYEQTFSVWMKRAQFGPHDWLSLPVPPGPSWLLVDTLEPETAYQFSVLAQNKLGTSAF | |
| SEVVTVITLAFPITTPEPLVLVTPPRCLIANRTQQGVLLSWLPPANHSFPIDRYIMEFRV | |
| AERWELLDDGIPGTEGEFFAKDLSQDTWYEFRVLAVMQDLIGEPSNIAGVSSTDIFPQPD | |
| LTEDGLARPVLAGIVATICFLAAAILFSTLAACFVNKQRKRKLKRKKDPPLSITHCRKSL | |
| ESPLSSGKVSPESIRTLRAPSESSDDQGQPAAKRMLSPTREKELSLYKKTKRAISSKKYS | |
| VAKAEAEAEATTPIELISRGPDGRFVMDPVEMEPSLKSRRIEGFPFAEETDMYPEFRQSD | |
| EENEDPLVPTSVAALKSQLTPLSSSQESYLPPPAYSPRFQPRGLEGPGGLEGRLQATGQA | |
| RPPAPRPFHHGQYYGYLSSSSPGEVEPPPFYVPEVGSPLSSVMSSPPLPTEGPFGHPTIP | |
| EENGENASNSTLPLTQTPTGGRSPEPWGRPEFPFGGLETPAMMFPHQLPPCDVPESLQPK | |
| AGLPRGLPPTSLQVPAAYPGILSLEAPKGWAGKSPGRGPVPAPPAAKWQDRPMQPLVSQG | |
| QLRHTSQGMGIPVLPYPEPAEPGAHGGPSTFGLDTRWYEPQPRPRPSPRQARRAEPSLHQ | |
| VVLQPSRLSPLTQSPLSSRTGSPELAARARPRPGLLQQAEMSEITLQPPAAVSFSRKSTP | |
| STGSPSQSSRSGSPSYRPAMGFTTLATGYPSPPPGPAPAGPGDSLDVFGQTPSPRRTGEE | |
| LLRPETPPPTLPTSGKLQRDRPAPATSPPERALSKL | |

A search of sequence databases reveals that the NOV45b amino acid sequence has 425 of 911 amino acid residues (46%) identical to, and 553 of 911 amino acid residues (60%) similar to, the 1189 amino acid residue ptnr:SPTREMBL-ACC:Q9P2J2 protein from Homo sapiens (Human) (KIAA1355 PROTEIN). Public amino acid databases include the GenBank databases, SwissProt, PDB and PIR.

NOV45b is expressed in at least prostate, brain (cerebellum). This information was derived by determining the tissue sources of the sequences that were included in the invention including but not limited to SeqCalling sources, Public EST sources, Literature sources, and/or RACE sources.

The disclosed NOV45a polypeptide has homology to the amino acid sequences shown in the BLASTP data listed in Table 45E.

TABLE 45E
BLAST results for NOV45

| Gene Index/ | | Length | Identity | Positives | |
|---|---|---|---|---|---|
| Identifier | Protein/Organism | (aa) | (%) | (%) | Expect |
| gi\|5689397\|dbj\|BAA82982.1\| | KIAA1030 protein | 763 | 761/763 | 761/763 | 0.0 |
| (AB028953) | [Homo sapiens] | | (99%) | (99%) | |
| gi\|15311046\|ref\|XP_ | KIAA1030 protein | 747 | 583/627 | 583/627 | 0.0 |
| 027486.2\| | [Homo sapiens] | | (92%) | (92%), | |
| (XM_027486) | | | | | |
| gi\|18578690\|ref\|XP_ | similar to KIAA1355 | 904 | 413/472 | 417/472 | 0.0 |
| 062186.2\| | protein [Homo sapiens] | | (87%) | (87%) | |
| (XM_062186) | | | | | |
| gi\|7243091\|dbj\|BAA92593.1\| | KIAA1355 protein | 1189 | 410/882 | 527/882 | e−179 |
| (AB037776) | [Homo sapiens] | | (46%) | (59%) | |
| gi\|18426807\|ref\|NP_ | neural cell | 1179 | 409/866 | 525/866 | e−177 |
| 291086.1\| | adhesion molecule | | (47%) | (60%) | |
| (NM_033608) | (Ncam)-like; | | | | |
| | KIAA1355 | | | | |
| | hypothetical | | | | |
| | protein (human); | | | | |
| | NCAM-like protein | | | | |
| | NRT1 [Mus | | | | |
| | musculus] | | | | |

Tables 45F-H list the domain descriptions from DOMAIN analysis results against NOV. This indicates that the NOV sequence has properties similar to those of other proteins known to contain this domain.

TABLE 45F
Domain Analysis of NOV45

gnl|Smart|smart00409, IG, Immunoglobulin
CD-Length = 86 residues, 100.0% aligned
Score = 62.4 bits (150), Expect = 2e−10

TABLE 45G
Domain Analysis of NOV45

gnl|Smart|smart00408, IGc2, Immunoglobulin C-2 Type
CD-Length = 63 residues, 92.1% aligned
Score = 54.7 bits (130), Expect = 4e−08

TABLE 45H
Domain Analysis of NOV45

gnl|Pfam|pfam00041, fn3, Fibronectin type III domain.
CD-Length = 86 residues, 90.7% aligned
Score = 43.5 bits (101), Expect = 8e−05

The basic structure of immunoglobulin (Ig) molecules is a tetramer of two light chains and two heavy chains linked by disulfide bonds. There are two types of light chains: kappa and lambda, each composed of a constant domain (CL) and a variable domain (VL). There are five types of heavy chains: alpha, delta, epsilon, gamma and mu, all consisting of a variable domain (VH) and three (in alpha, delta and gamma) or four (in epsilon and mu) constant domains (CH1 to CH4). The major histocompatibility complex (MHC) molecules are made of two chains. In class I the alpha chain is composed of three extracellular domains, a transmembrane region and a cytoplasmic tail. The beta chain (beta-2-microglobulin) is composed of a single extracellular domain. In class II, both the alpha and the beta chains are composed of two extracellular domains, a transmembrane region and a cytoplasmic tail. It is known that the Ig constant chain domains and a single extracellular domain in each type of MHC chains are related. These homologous domains are approximately one hundred amino acids long and include a conserved intradomain disulfide bond. Members of the immunoglobulin superfamily are found in hundreds of proteins of different functions. Examples include antibodies, the giant muscle kinase titin and receptor tyrosine kinases. Immunoglobulin-like domains may be involved in protein-protein and protein-ligand interactions.

Fibronectins are multi-domain glycoproteins found in a soluble form in plasma, and in an insoluble form in loose connective tissue and basement membranes. They contain multiple copies of 3 repeat regions (types I, II and III), which bind to a variety of substances including heparin, collagen, DNA, actin, fibrin and fibronectin receptors on cell surfaces. The wide variety of these substances means that fibronectins are involved in a number of important functions: e.g., wound healing; cell adhesion; blood coagulation; cell differentiation and migration; maintenance of the cellular cytoskeleton; and tumour metastasis. The role of fibronectin in cell differentiation is demonstrated by the marked reduction in the expression of its gene when neoplastic transformation occurs. Cell attachment has been found to be mediated by the binding of the tetrapeptide RGDS to integrins on the cell surface, although related sequences can also display cell adhesion activity. Plasma fibronectin occurs as a diner of 2 different subunits, linked together by 2 disulphide bonds near the C-terminus. The difference in the 2 chains occurs in the type III repeat region and is caused by alternative splicing of the mRNA from one gene. The observation that, in a given protein, an individual repeat of one of the 3 types (e.g., the first FnIII repeat) shows much less similarity to its subsequent tandem repeats within that protein than to its equivalent repeat between fibronectins from other species, has suggested that the repeating structure of fibronectin arose at an early stage of evolution. It also seems to suggest that the structure is subject to high selective pressure. The fibronectin type III repeat region is an approximately 100 amino acid domain, different tandem repeats of which contain binding sites for DNA, heparin and the cell surface. The superfamily of sequences believed to contain FnIII repeats represents 45 different families, the majority of which are involved in cell surface binding in some manner, or are receptor protein tyrosine kinases, or cytokine receptors.

The disclosed NOV45 nucleic acid of the invention encoding a Ig/fibronectin domain-like protein includes the nucleic acid whose sequence is provided in Table 45A or 45C or a fragment thereof. The invention also includes a mutant or variant nucleic acid any of whose bases may be changed from the corresponding base shown in Table 45A or 45C while still encoding a protein that maintains its Ig/fibronectin domain-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV45 protein of the invention includes the Ig/fibronectin domain-like protein whose sequence is provided in Table 45B or 45D. The invention also includes a mutant or variant protein any of whose residues may be changed from the corresponding residue shown in Table 45B or 45D while still encoding a protein that maintains its Ig/fibronectin domain-like activities and physiological functions, or a functional fragment thereof. In the mutant or variant protein, up to about 54 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Ig/fibronectin domain-like protein (NOV45) may function as a member of a “Ig/fibronectin domain family”. Therefore, the NOV45 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV45 nucleic acids and proteins of the invention are useful in potential therapeutic applications implicated in diseases including but not limited to various pathologies and disorders as indicated below. For example, a cDNA encoding the Ig/fibronectin domain-like protein (NOV45) may be useful in gene therapy, and the Ig/fibronectin domain-like protein (NOV45) may be useful when administered to a subject in need thereof. By way of nonlimiting example, the compositions of the present invention will have efficacy for treatment of patients suffering from Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, fertility, or other pathologies or conditions. The NOV45 nucleic acid encoding the Ig/fibronectin domain-like protein of the invention, or fragments thereof, may further be useful in diagnostic applications, wherein the presence or amount of the nucleic acid or the protein are to be assessed.

NOV45 nucleic acids and polypeptides are further useful in the generation of antibodies that bind immuno-specifically to the novel NOV45 substances for use in therapeutic or diagnostic methods. These antibodies may be generated according to methods known in the art, using prediction from hydrophobicity charts, as described in the “Anti-NOVX Antibodies” section below. The disclosed NOV45 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV46

A disclosed NOV46 nucleic acid of 1247 nucleotides (also referred to as CG57682-01) encoding a G2/mitotic-specific cyclin B2-like protein is shown in Table 46A. The start and stop codons are in bold letters.

TABLE 46A
NOV46 nucleotide sequence.

(SEQ ID NO:109)

ATCCACATTGCATTTACTTGTCAGGTATGCTATTAGGACTCTCCAAAGAGACAGAACCAATAGGGGAAAT
GGGTATATGCAGAGACAGAGAGACAGGAGTTAATTCTAAACCTAAGAGTCATGTGACTATTAGGCATTCC
ATTTTAGAAAAAATTGGAAATAGAGTTACAGCCAGAGCAGCACAAGTAGCTAAGAAAGCTCAGAACACAC
AAAGTGCCAGTTCAACCCAGGGAAACAACAAATGTCAACAAACAACTGAAACCTACTGCTTCTGTGAAAC
AGTACAGATGGAAATGTTGGCTCCAAAGGGTCCTTCTCCCATACCTGAGGATGCCTCCGTGAAAGAAGAG
AACATCTGCCGAGCTTTTTCTGATGCTTTGCTCTACAAAATTGAGGATATTGATAACAAAGATTGGAATA
ACCCTCAGCTCTGCAGTGACTATTTAAGGAAGGGTATCTATCAGTACCTCAGGCAGCTGGAGATTTTGCA
GTTCATAAACCCACATGTCTTAGGTGGAGGAGATGTAAATGGACATAAGCATACCATCCTGGTAGACTGG
TTGGTGCAAATCCACTCCAAGTTTAGGCTTCTTCAGGAGACTCTGTATGTGTGTGTTGCCATTATGGATG
GATCTTTACTGGTTCAGCCAGTTTCCCAGAGGAAGCTTCAACTAGTTTGGATTACTGCTCTGCTCTTGGC
TTCCAAGTATGAGGAGATGTTTTCTCCAAATACTGAAGGCTTTGTTTACATCACAGACAATGCTTATACT
AGTTTCCAAATCCAAGAAATGGAAACTCTAATTTTGAAAGAACTGAAATTTGAGGTGGGTGGACCCTTGC
CACTACACTTCTTAAGGCAAGCATCAAAAGCCGGGAAGGCTGATGTTGAACAGCACACTTTAGCCAAATA
TTTGATGGAGCTGACTCTCATTGACTACGATATGATGCATTATCATCCTTCTAAGGCAGCAACAGCTGCT
TCCTGCTTGTCTCAGAAGGTTCTGGGCCAAGGAAAATGGAACTTAAAGCAGCAGTGTTATACAGGATACA
CACAGAATGAAGTATTGGAAGTCATGCAGCACGTGGCCAAAAATGTGCTGAAAGTAAATGAAAACTTAAC
TAAATTCATCGCCATCAAGAATAAGTATGCAAGCAACAAATTCCTGAAGATCAGCATGATCCCTCAGCTG
AACTCAAAAGCCATCAAAGACCTTGCCTTCCCTCTGATGGGAGGGTCCTAGGCTCCA

In a search of public sequence databases, the NOV46 nucleic acid sequence, located on chromsome 7 has 1068 of 1172 bases (91%) identical to a gb:GENBANK-ID:HSM800659|acc:AL080146.1 mRNA from Homo sapiens (Homo sapiens mRNA; cDNA DKFZp434B174 (from clone DKFZp434B174); complete cds). Public nucleotide databases include all GenBank databases and the GeneSeq patent database.

The disclosed NOV46 polypeptide (SEQ ID NO:110) encoded by SEQ ID NO:109 has 404 amino acid residues and is presented in Table 46B using the one-letter amino acid code. Signal P, Psort and/or Hydropathy results predict that NOV46 has a signal peptide and is likely to be localized in the cytoplasm with a certainty of 0.6500.

TABLE 46B
Encoded NOV46 protein sequence.

| | |
|---|---|
| MLLGLSKETEPIGEMGICRDRETGVNSKPKSHVTIRHSILEKIGNRVTARAAQVAKKAQN | (SEQ ID NO:110) |
| TQSASSTQGNNKCQQTTETYCFCETVQMEMLAPKGPSPIPEDASVKEENICRAFSDALLY | |
| KIEDIDNKDWNNPQLCSDYLRKGIYQYLRQLEILQFINPHVLGGGDVNGHKHTILVDWLV | |
| QIHSKFRLLQETLYVCVAIMDGSLLVQPVSQRKLQLVWITALLLASKYEEMFSPNTEGFV | |
| YITDNAYTSFQIQEMETLILKELKFEVGGPLPLHFLRQASKAGKADVEQHTLAKYLMELT | |
| LIDYDMMHYHPSKAATAASCLSQKVLGQGKWNLKQQCYTGYTQNEVLEVMQHVAKNVLKV | |
| NENLTKFIAIKNKYASNKFLKISMIPQLNSKAIKDLAFPLMGGS | |

A search of sequence databases reveals that the NOV46 amino acid sequence has 303 of 383 amino acid residues (79%) identical to, and 333 of 383 amino acid residues (86%) similar to, the 398 amino acid residue ptnr:SWISSNEW-ACC:O95067 protein from Homo sapiens (Human) (G2/MITOTIC-SPECIFIC CYCLIN B2). Public amino acid databases include the GenBank databases, SwissProt, PDB and PIR.

NOV46 is expressed in at least Adrenal gland, Aorta, B-cells, Blood, Bone, Brain, Breast, CNS, Colon, Ear, Esophagus, Eye, Gall bladder, Germ Cell, Head and neck, Heart, Kidney, Larynx, Liver, Lung, Lymph, Marrow, Muscle, Neural, Omentum, Ovary, Pancreas, Parathyroid, Peripheral nervous system, Placenta, Pooled, Prostate, Skin, Small intestine, Spleen, Stomach, Synovial membrane, Testis, Tissue culture, Tonsil, Uterus, Whole embryo, and adrenal gland. This information was derived by determining the tissue sources of the sequences that were included in the invention including but not limited to SeqCalling sources, Public EST sources, Literature sources, and/or RACE sources.

The disclosed NOV46 polypeptide has homology to the amino acid sequences shown in the BLASTP data listed in Table 46C.

TABLE 46C
BLAST results for NOV46

| Gene Index/ | | Length | Identity | Positives | |
|---|---|---|---|---|---|
| Identifier | Protein/Organism | (aa) | (%) | (%) | Expect |
| gi\|4757930\|ref\|NP_004692.1\| | cyclin B2 [Homo | 398 | 303/387 | 333/387 | e−67 |
| (NM_004701) | sapiens ] | | (78%) | (85%) | |
| gi\|5921730\|sp\|O77689\| | G2/mitotic- | 398 | 285/385 | 328/385 | e−62 |
| CGB2_BOVIN | specific cyclin | | (74%) | (85%) | |
| | B2 | | | | |
| gi\|584914\|sp\|P37883\| | G2/mitotic- | 397 | 288/385 | 321/385 | e−58 |
| CGB2_MESAU | specific cyclin | | (74%) | (82%) | |
| | B2 | | | | |
| gi\|14198371\|gb\|AAH08247.1\| | Similar to cyclin | 398 | 278/382 | 316/382 | e−51 |
| AAH08247 | B2 [Mus musculus] | | (72%) | (81%) | |
| (BC008247) | | | | | |
| gi\|6680866\|ref\|NP_031656.1\| | cyclin B2 [Mus | 398 | 274/382 | 313/382 | e−49 |
| (NM_007630) | musculus] | | (71%) | (81%) | |

Tablez 46D-E list the domain descriptions from DOMAIN analysis results against NOV46. This indicates that the NOV46 sequence has properties similar to those of other proteins known to contain this domain.

TABLE 46D
Domain Analysis of NOV46

gnl|Pfam|pfam00134, cyclin, Cyclin, N-terminal domain. Cyclins
regulate cyclin dependent kinases (CDKs). Cyclins contain two domains
of similar all-alpha fold, of which this family corresponds with the
N-terminal domain.
CD-Length = 128 residues, 99.2% aligned
Score = 119 bits (298), Expect = 3e−28

TABLE 46E
Domain Analysis of NOV46

gnl|Smart|smart00385, CYCLIN, domain present in cyclins, TFIIB and
Retinoblastoma; A helical domain present in cyclins and TFIIB (twice)
and Retinoblastoma (once). A protein recognition domain functioning in
cell-cycle and transcription control.
CD-Length = 83 residues, 100.0% aligned
Score = 62.8 bits (151), Expect = 4e−11

Two B-type cyclins, B1 and B2, have been identified in mammals. Proliferating cells express both cyclins, which bind to and activate p34(cdc2). To test whether the two B-type cyclins have distinct roles, lines of transgenic mice were generated, one lacking cyclin Bi and the other lacking cyclin B2. Cyclin B1 proved to be an essential gene; no homozygous BI-null pups were born. In contrast, nullizygous B2 mice developed normally and did not display any obvious abnormalities. Both male and female cyclin B2-null mice were fertile, which was unexpected in view of the high levels and distinct patterns of expression of cyclin B2 during spermatogenesis. The expression of cyclin B1 overlaps the expression of cyclin B2 in the mature testis, but not vice versa. Cyclin B1 can be found both on intracellular membranes and free in the cytoplasm, in contrast to cyclin B2, which is membrane-associated. These observations suggest that cyclin B1 may compensate for the loss of cyclin B2 in the mutant mice, and implies that cyclin B 1 is capable of targeting the p34(cdc2) kinase to the essential substrates of cyclin B2.

The disclosed NOV46 nucleic acid of the invention encoding a G2/mitotic-specific cyclin B2-like protein includes the nucleic acid whose sequence is provided in Table 46A or a fragment thereof. The invention also includes a mutant or variant nucleic acid any of whose bases may be changed from the corresponding base shown in Table 46A while still encoding a protein that maintains its G2/mitotic-specific cyclin B2-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 9 percent of the bases may be so changed.

The disclosed NOV46 protein of the invention includes the G2/mitotic-specific cyclin B2-like protein whose sequence is provided in Table 46B. The invention also includes a mutant or variant protein any of whose residues may be changed from the corresponding residue shown in Table 46B while still encoding a protein that maintains its G2/mitotic-specific cyclin B2-like activities and physiological functions, or a functional fragment thereof. In the mutant or variant protein, up to about 21 percent of the residues may be so changed.

The invention further encompasses antibodies and antibody fragments, such as F_{ab} or (F_{ab})₂, that bind immunospecifically to any of the proteins of the invention.

The above defined information for this invention suggests that this G2/mitotic-specific cyclin B2-like protein (NOV46) may function as a member of a “G2/mitotic-specific cyclin B2 family”. Therefore, the NOV46 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/genie ablation), research tools, tissue regeneration in vivo and in vitro of all tissues and cell types composing (but not limited to) those defined here.

The NOV46 nucleic acids and proteins of the invention are useful in potential therapeutic applications implicated in diseases including but not limited to various pathologies and disorders as indicated below. For example, a cDNA encoding the G2/mitotic-specific cyclin B2-like protein (NOV46) may be useful in gene therapy, and the G2/mitotic-specific cyclin B2-like protein (NOV46) may be useful when administered to a Subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Adrenoleukodystrophy, Congenital Adrenal Hyperplasia, Hemophilia, Hypercoagulation, Idiopathic thrombocytopenic purpura, Immunodeficiencies, Graft vesus host, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, or other pathologies or conditions. The NOV46 nucleic acid encoding the G2/mitotic-specific cyclin B2-like protein of the invention, or fragments thereof, may further be useful in diagnostic applications, wherein the presence or amount of the nucleic acid or the protein are to be assessed.

NOV46 nucleic acids and polypeptides are further useful in the generation of antibodies that bind immuno-specifically to the novel NOV46 substances for use in therapeutic or diagnostic methods. These antibodies may be generated according to methods known in the art, using prediction from hydrophobicity charts, as described in the “Anti-NOVX Antibodies” section below. The disclosed NOV46 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV47

A disclosed NOV47 nucleic acid of 15645 nucleotides (also referred to as CG57764-01) encoding a ALR-like protein is shown in Table 47A. The start and stop codons are in bold letters.

TABLE 47A
NOV47 nucleotide sequence.

| | |
|---|---|
| ATGTCCCCTCCACCTGAAGAGTCACCCATGTCTCCACCACCGGAGGCATCTCGTCTGTTC | (SEQ ID NO:111) |
| CCACCATTTGAAGAGTCTCCTCTGTCCCCTCCACCTGAGGAGTCTCCCCTTTCCCCACCA | |
| CCTGAGGCATCACGCCTGTCCCCACCACCTGAGGACTCGCCTATGTCCCCACCACCTGAA | |
| GAATCACCTATGTCCCCCCCACCTGAGGTATCGCGCCTATCCCCCCTGCCTGTGGTGTCA | |
| CGCCTGTCTCCACCGCCTGAGGAATCTCCCTTGTCCCCACCGCCTGAGGAGTCTCCCACG | |
| TCCCCTCCACCTGAGGCTTCACGCCTCTCCCCACCACCTGAGGACTCCCCCACATCCCCA | |
| CCACCTGAGGACTCACCTGCTTCCCCACCACCGGAGGACTCGCTCATGTCCCTGCCGCTG | |
| GAGGAGTCACCCCTGTTGCCACTACCTGAGGAGCCGCAACTCTGCCCCCGGTCCGAGGGG | |
| CCGCACCTGTCACCCCGGCCTGAGGAGCCGCACCTGTCCCCCCGGCCTGAGGACCCACAC | |
| CTATCTCCGCAGGCTGAGGAGCCACACCTGTCCCCCCACCCTGAGGACCCATGCCTATGC | |
| GCTGTGCCTGAGGAGCCACACTTGTCCCCCCAGGCTGAGGGACCACATCTGTCCCCTCAG | |
| CCTGAGGAATTGCACCTGTCCCCCCAGACTGAGGAGCCGCACCTGTCTCCTGTGCCTGAG | |
| GAGCCATGCTTGTCCCCCCAACCTGAGGAATCACACCTGTCCCCCCAGTCTGAGGAGCCA | |
| TGCCTGTCCCCCCGGCCTGAGGAATCGCATCTGTCCCCTGAGCTTGAGAAGCCACCCCTG | |
| TCCCCTCGGCCTGAAAAGCCCCCTGAGGAGCCAGGCCAATGCCCTGCACCTGAGGAGCTG | |
| CCCTTGTTCCCTCCCCCTGGGGAACCATCCTTATCTCCCTTGCTTGGAGAGCCAGCCCTG | |
| TCTGAGCCTGGGGAACCACCTCTGTCCCCTCTGCCCGAGGAGCTGCCGTTGTCCCCATCT | |
| GGGGAGCCATCCTTGTCGCCTCAGCTGATGCCACCAGATCCCCTTCCTCCTCCACTCTCA | |
| CCCATTATCACAGCTGCGGCCCCACCGGCCCTGTCTCCTTTGGGGGAGTTAGAGTACCCC | |
| TTTGGTGCCAAAGGGGACAGTGACCCTGAGTCACCGTTGGCTGCCCCCATCCTGGAGACA | |
| CCCATCAGCCCTCCACCAGAAGCTAACTGCACTGACCCTGAGCCTGTCCCCCCTATGATC | |
| CTTCCCCCATCTCCAGGCTCCCCAGTGGGGCCGGCTTCTCCCATCCTGATGGAGCCCCTT | |
| CCTCCTCAGTGTTCGCCACTCCTTCAGCATTCCCTGGTTCCCCAAAACTCCCCTCCTTCC | |
| CAGTGCTCTCCTCCTGCCCTACCACTGTCCGTTCCCTCCCCGTTGAGTCCCATAGGGAAG | |
| GTAGTGGGGGTCTCAGATGAGGCTGAGCTGCACGAGATGGAGACTGAGAAAGTTTCAGAA | |
| CCTGAATGCCCAGCCTTGGAACCCAGTGCCACCAGTCCTCTCCCTTCCCCAATGGGGGAC | |
| CTTTCCTGCCCCGCCCCCAGCCCTGCCCCAGCCCTGGATGACTTCTCTGGCCTAGGGGAA | |
| GACACAGCCCCTCTGGATGGGATTGATGCTCCGGGTTCACAGCCAGAGCCTGGACAGACC | |
| CCTGGCAGTTTGGCTAGTGAACTTAAAGGCTCCCCTGTGCTCCTGGACCCCGAGGAGCTG | |
| GCCCCTGTGACCCCTATGGAGGTCTACCCCGAATGCAAGCAGACAGCAGGGCGGGGCTCA | |
| CCATGTGAAGAACAGGAAGAGCCACGTGCACCGGTGGCCCCCACACCACCCACTCTCATC | |
| AAATCCGACATCGTTAACGAGATCTCTAATCTGAGCCAGGGTGATGCCAGTGCCAGTTTT | |
| CCTGGCTCAGAGCCCCTCCTGGGCTCTCCAGACCCGGAGGGGGGTGGCTCCCTGTCCATG | |
| GAGTTGGGGGTCTCTACGGATGTTAGTCCAGCCCGAGATGAGGGCTCCCTACGGCTCTGT | |
| ACTGACTCACTGCCAGAGACTGATGACTCACTATTGTGCGATGCTGGGACAGCTATCAGC | |
| GGAGGCAAAGCTGAGGGGGAGAAGGGGCGGCGGCGCAGCTCCCCAGCCCGTTCCCGCATC | |
| AAACAGGGTCGCAGCAGCAGTTTCCCAGGAAGACGCCGGCCTCGTGGAGGAGCCCATGGA | |
| GGGCGTGGTAGAGGACGGGCCCGGCTAAAGTCAACTGCTTCTTCCATTGAGACTCTGGTA | |
| GTTGCTGACATTGATAGCTCTCCCAGTAAGGAGGAGGAGGAAGAAGATGATGACACCATG | |
| CAGAATACCGTGGTTCTCTTCTCCAACACAGACAAATTTGTCCTAATGCAGGACATGTGT | |
| GTGGTATGTGGCAGCTTTGGCCGGGGGGCAGAGGGCCACCTCCTTGCCTGTTCGCAGTGC | |
| TCTCAGTGCTATCACCCTTACTGTGTCAACAGCAAGATCACCAAGGTGATGCTGCTCAAG | |
| GGCTGGCGTTGTGTGGAGTGTATTGTGTGTGAGGTGTGTGGCCAGGCCTCCGACCCCTCA | |
| CGCCTGCTGCTCTGTGATGACTGTGATATTAGCTACCACACATACTGCCTGGACCCCCCA | |
| CTGCTCACCGTCCCCAAGGGCGGCTGGAAGTGCAAGTGGTGTGTGTCCTGTATGCAGTGT | |
| GGGGCTGCTTCCCCTGGCTTCCACTGTGAATGGCAGAATAGTTACACACACTGTGGGCCC | |
| TGTGCCAGCCTGGTGACCTGCCCTATCTGTCATGCTCCTTACGTAGAAGAGGACCTACTA | |
| ATCCAGTGCCGCCACTGTGAACGGTGGATGCATGCAGGCTGTGAGAGCCTCTTCACAGAG | |
| GACGATGTGGACCACGCACCCGATGAAGGCTTTGACTGTGTCTCCTGCCAGCCCTACGTG | |
| GTAAAGCCTGTGGCGCCTGTTGCACCTCCAGAGCTGGTGCCCATGAAGGTGAAAGAGCCA | |
| GAGCCCCAGTACTTTCGCTTCGAAGGCGTGTGGCTGACAGAAACTGGCATGGCCTTGCTG | |
| CGTAACCTGACCATGTCACCACTGCACAAGCGGCGCCAACGGCGAGGACGGCTTGGCCTC | |
| CCAGGCGAGGCAGGATTGGAGGGTTCTGAGCCCTCAGATGCCCTTGGCCCTGATGACAAG | |
| AAGGATGGGGACCTGGACACCGATGAGCTGCTCAAGGGTGAAGGTGGTGTGGAGCACATG | |
| GAGTGCGAAATTAAACTGGAGGGCCCCGTCAGCCCTGATGTGGAGCCTGGCAAAGAGGAG | |
| ACCGAGGAAAGCAAAAAACGCAAGCGTAAACCATATCGGCCTGGCATTGGTGGTTTCATG | |
| GTGCGACAGCGGAAATCCCACACACGCACGAAAAAGGGGCCTGCTGCACAGGCGGAGGTG | |
| TTGAGTGGGGATGGGCAGCCCGACGAGGTGATACCTGCTGACCTGCCTGCAGAGGGCGCC | |
| GTGGAGCAGAGCTTAGCTGAAGGGGATGAGAAGAAGAAGCAACAGCGGCGAGGGCGCAAG | |
| AGGAGCAAACTGGAGGGCATGTTCCCTGCTTACTTGCAGGAAGCCTTCTTTGGGAAGGAG | |
| CTGCTGGACCTGAGCCGTAAGGCCCTTTTTGCAGTTGGGGTGGGCCGGCCAAGCTTTGGA | |
| CTAGGGACCCCAAAAGCCAAGGGAGATGGAGGCTCAGAAAGGAAGGAACTCCCCACATCG | |
| CAGAAAGGAGATGATGGTCCAGATATTGCAGATGAAGAATCCCGTGGCCTCGAGGGCAAA | |
| GCCGATACACCAGGACCTGAGGATGGGGGCGTGAAGGCATCCCCAGTGCCCAGTGACCCT | |
| GAGAAGCCAGGCACCCCAGGTGAAGGGATGCTTAGCTCTGACTTAGACAGGATTTCCACA | |
| GAAGAACTGCCCAAGATGGAATCCAAGGACCTGCAGCAGCTCTTCAAGGATGTTCTGGGC | |
| TCTGAACGAGAACAGCATCTGGGTTGTGGAACCCCTGGCCTAGAAGGCAGCCGTACGCCA | |
| CTGCAGAGGCCCTTTCTTCAAGGTGGACTCCCTTTGGGCAATCTGCCCTCCAGCAGCCCA | |
| ATGGACTCCTACCCAGGCCTCTGCCAGTCCCCGTTCCTGGATTCTAGGGAGCGCGGGGGC | |
| TTCTTTAGCCCGGAACCCGGTGAGCCCGACAGCCCCTGGACGGGCTCAGGTGGCACCACG | |
| CCCTCCACCCCCACAACCCCCACCACGGAGGGTGAGGGCGACGGACTCTCCTATAACCAG | |
| CGGAGTCTTCAGCGCTGGGAGAAGGATGAGGAGTTGGGCCAGCTGTCCACCATCTCGCCT | |
| GTGCTCTATGCCAACATTAATTTTCCTAATCTCAAGCAAGACTACCCAGACTGGTCAAGC | |
| CGTTGCAAACAAATCATGAAGCTCTGGAGAAAGGTTCCAGCAGCTGACAAAGCCCCCTAC | |
| CTGCAAAAGGCCAAAGATAACCGGGCAGCTCACCGCATCAACAAGGTGCAGAAGCAGGCT | |
| GAGAGCCAGATCAACAAGCAGACCAAGGTGGGCGACATAGCCCGTAAGACTGACCGACCG | |
| GCCCTACATCTCCGCATTCCCCCGCAGCCAGGGGCACTGGGCAGCCCGCCCCCCGCTGCT | |
| GCCCCCACCATTTTCATTGGCAGCCCCACTACCCCCGCCGGCTTGTCTACCTCTGCGGAC | |
| GGGTTCCTGAAGCCGCCGGCGGGCTCGGTGCCTGGCCCTGACTCGCCTGGTGAGCTCTTC | |
| CTCAAGCTCCCACCCCAGGTGCCCGCCCAAGCGCCTTCGCAGGACCCCTTTGGACTGGCC | |
| CCTGCCTATCCCCTGGAGCCCCGCTTCCCCACGGCACCGCCCACCTATCCCCCCTATCCT | |
| AGTCCTACGGGGGCCCCTGCGCAGCCCCCGATGCTGGGCGCCTCATCTCGTCCTGGGGCT | |
| GGCCAGCCAGGGGAATTCCACACTACCCCACCTGGCACCCCCAGACACCAGCCCTCCACA | |
| CCTGACCCGTTCCTCAAACCCCGCTGCCCCTCGCTGGATAACTTGGCTGTGCCTGAGAGC | |
| CCTGGGGTAGGGGGAGGCAAAGCTTCCGAGCCCCTGCTCTCGCCCCCACCTTTTGGGGAG | |
| TCCCGGAAGGCCCTAGAGGTGAAGAAGGAAGAGCTTGGGGCATCCTCTCCTAGCTATGGG | |
| CCCCCAAACCTGGGCTTTGTTGACTCACCCTCCTCAGGCACCCACCTGGGTGGCCTGGAG | |
| TTAAAGACACCTGATGTCTTCAAAGCCCCCCTGACCCCTCGGGCATCTCAGGTAGAGCCC | |
| CAGAGCCCGGGCTTGGGCCTAAGGCCCCAGGAGCCACCCCCTGCCCAGCCTTTGGCACCT | |
| TCTCCTCCAAGTCACCCAGACATCTTTCGCCCTGGCTCCTACACTGACCCATATGCTCAG | |
| CCCCCATTGACTCCTCGGCCCCAACCTCCGCCCCCTGAGAGCTGCTGTGCTCTGCCCCCT | |
| CGCTCACTGCCCTCCCACCCTTTCTCCCGAGTGCCTGTCAGTCCTCAGTCCCAGTCCAGC | |
| TCCCAGTCTCCACTGACACCCCGGCCTCTGTCTGCTGAAGCTTTTTGCCCATCACCCGTT | |
| ACCCCTCGCTTCCAGTCCCCTGACCCTTATTCTCGCCCACCCTCACGCCCTCAGTCCCGT | |
| GACCCATTTGCCCCATTGCATAAGCCACCCCGACCCCAGCCCCCTGAAGTTGCCTTTAAG | |
| GCTGGGTCTCTAGCCCACACTTCGCTGGGGGCTGGCGGGTTCCCAGCAGCCCTGCCCGCG | |
| GGGCCAGCAGGTGAGCTCCATGCCAAGGTCCCAAGTGGGCAGCCCCCCAATTTTGTCCGG | |
| TCCCCTGGGACGGGTGCATTTGTGGGCACCCCCTCTCCCATGCGTTTCACTTTCCCTCAG | |
| GCAGTAGGGGAGCCTTCCCTAAAGCCCCCTGTCCCTCAGCCTGGTCTCCCGCCACCCCAT | |
| GGGATCAACAGCCATTTTGGGCCCGGCCCCACCTTGGGCAAGCCTCAAAGCACAAACTAC | |
| ACAGTAGCCACAGGGAACTTCCACCCATCGGGCAGCCCCCTGGGGCCCAGCAGCGGGTCC | |
| ACAGGGGAGAGCTATGGGCTGTCCCCACTACGCCCTCCGTCGGTTCTGCCACCACCTGCA | |
| CCCGACGGATCCCTCCCCTACCTGTCCCATGGAGCCTCACAGCGATCAGGCATCACCTCT | |
| CCTGTCGAAAAGCGAGAAGACCCAGGGACTGGAATGGGTAGCTCTTTGGCGACAGCTGAA | |
| CTCCCAGGTACCCAGGACCCAGGCATGTCCGGCCTTAGCCAAACAGAGCTGGAGAAGCAA | |
| CGGCAGCGCCAGCGGCTACGAGAGCTGCTGATTCGGCAGCAGATCCAGCGCAACACCCTG | |
| CGGCAGGAGAAGGAAACAGCTCCAGCAGCTGCGGGAGCAGTGGGGCCTCCAGGCAGCTGG | |
| GGTGCTGAGCCCAGCAGCCCTGCCTTTGAGCAGCTGAGTCGAGGCCAGACCCCCTTTGCT | |
| GGGACACAGGACAAGAGCAGCCTTGTGGGGTTGCCCCCAAGCAAGCTGAGTGGCCCCATC | |
| CTGGGGCCAGGGTCCTTCCCTAGCGATGACCGACTCTCCCGGCCACCTCCACCAGCCACG | |
| CCTTCCTCTATGGATGTGAACAGCCGGCAACTGGTAGGAGGCTCCCAAGCTTTCTATCAG | |
| CGAGCACCCTATCCTGGGTCCCTGCCCTTACAGCAGCAACAGCAACAACTGTGGCAGCAA | |
| CAACAGGCAACAGCAGCAACCTCCATGCGATTTGCCATGTCAGCTCGCTTTCCATCAACT | |
| CCTGGACCTGAACTTGGCCGCCAAGCCCTAGGTTCCCCGTTGGCGGGAATTTCCACCCGT | |
| CTGCCAGGCCCTGGTGAGCCAGTGCCTGGTCCAGCTGGTCCTGCCCAGTTCATTGAGCTG | |
| CGGCACAATGTACAGAAAGGACTGGGACCTGGGGGCACTCCGTTTCCTGGTCAGGGCCCA | |
| CCTCACAGACCCCGTTTTTACCCTGTAAGTGAGGACCCCCACCGACTGGCTCCTGAAGGG | |
| CTTCGGGGCCTGGCGCTATCAGGTCTTCCCCCACAGAAACCCTCAGCCCCACCGGCCCCT | |
| GAATTGAACAACAGTCTTCATCCAACACCCCACACCAAGGGTCCTACCCTGCCAACTGGT | |
| TTGGAGCTGGTCAACCGGCCCCCGTCGAGCACTGAGCTTGGCCGCCCCAATCCTCTGGCC | |
| CTGGAAGCTGGGAAGTTGCCCTGTGAGGATCCCGAGCTGGATGACGATTTTGATGCCCAC | |
| AAGGCCCTAGAGGATGATGAAGAGCTTGCTCACCTGGGTCTGGGTGTGGATGTGGCCAAG | |
| GGTGATGATGAACTTGGCACCTTAGAAAACCTGGAGACCAATGACCCCCACTTGGATGAC | |
| CTGCTCAATGGAGACGAGTTTGACCTGCTGGCATATACTGATCCTGAGCTGGACACTGGG | |
| GACAAGAAGGATATCTTCAATGAGCACCTGAGGCTGGTAGAATCGGCTAATGAGGAGGCT | |
| GAACGGGAGGCCCTGCTGCGGGGGGTGGAGCCAGGACCCTTGGGCCCTGAGGAGCGCCCT | |
| CCCCCTGCTGCTGATGCCTCTGAACCCCGCCTGGCATCTGTGCTCCCTGAGGTGAAGCCC | |
| AAGGTGGAGGAGGGTGGACGCCACCCTTCTCCTTGCCAATTCACCATTGCTACCCCCAAG | |
| GTAGAGCCCGCACCTGCTGCCAATTCCCTTGGCCTGGGGCTAAAGCCAGGACAGAGCATG | |
| ATGGGCAGCCGGGATACCCGGATGGGCACAGGGCCATTTTCTAGCAGTGGGCACACAGCT | |
| GAGAAGGCCTCCTTTGGGGCCACGGGAGGGCCACCAGCTCACCTGCTGACCCCCAGCCCA | |
| CTGAGTGGCCCAGGAGGATCCTCCCTGCTGGAAAAGTTTGAGCTCGAGAGTGGGGCTTTG | |
| ACCTTGCCTGGTGGACCTGCAGCATCTGGGGATGAGCTAGACAAGATGGAGAGCTCACTG | |
| GTAGCCAGCGAGTTACCCCTGCTCATTGAGGACCTGTTGGAGCATGAGAAGAAGGAGCTG | |
| CAGAAGAAGCAGCAGCTTTCAGCACAGTTGCAGCCTGCCCAGCAGCAGCAGCAACAGCAG | |
| CAGCAGCATTCCCTACTGCCTGCACCAGGCCCTGCCCAGGCCATGTCTTTGCCACATGAG | |
| GGCTCTTCTCCCAGTTTGGCTGGGTCCCAACAGCAGCTTTCCCTGGGTCTTGCAGTTGCC | |
| CGACAGCCAGGTTTGCCCCAGCCACTGATGCCCACCCAGCCACCAGCTCATGCCCTCCAG | |
| CAACGCCTGGCTCCATCCATGGCTATGGTGTCCAATCAAGGGCATATGCTAAGTGGGCAG | |
| CATGGAGGGCAGGCAGGCTTGGTACCCCAGCAGAGCTCACAGCCAGTGCTATCACAGAAG | |
| CCCATGGGCACCATGCCACCTTCCATGTGCATGAAGCCGCAGCAATTGGCAATGCAGCAG | |
| CAGCTGGCAAACAGCTTCTTCCCAGATACAGACCTGGACAAATTTGCTGCAGAAGATATC | |
| ATTGGTCCCATTGCAAAGGCCAAGATGGTGGCTTTGAAAGGCATCAAGAAAGTGATGGCT | |
| CAGGGCAGCATTGGGGTGGCACCTGGTATGAACAGACAGCAAGTGTCTCTGCTAGCCCAG | |
| AGGCTCTCGGGGGGACCTAGCAGTGATCTGCAGAACCATGTGGCAGCTGGGAGTGGCCAG | |
| GAGCGGAGTGCTGGTGATCCCTCCCAGCCTCGTCCCAACCCGCCCACTTTTGCTCAGGGA | |
| GTGATCAATGAAGCTGACCAGCGGCAGTATGAGGAGTGGCTGTTCCATACCCAGCAGCTC | |
| CTACAGATGCAGCTGAAGGTGCTAGAGGAGCAGATTGGTGTACACCGCAAGTCCCGGAAG | |
| GCTCTGTGTGCCAAGCAGCGCACTGCCAAAAAAGCTGGCCGTGAGTTCCCAGAAGCTGAT | |
| GCTGAGAAGCTCAAGCTGGTTACAGAGCAGCAGAGCAAGATCCAGAAACAACTGGATCAG | |
| GTCCGGAAACAGCAGAAGGAGCACACTAATCTCATGGCAGAATATCGGAACAAGCAGCAG | |
| CAACAACAGCAGCAGCAGCAGCAACAACAGCAACAGCACTCAGCTGTGCTGGCTCTCAGC | |
| CCTTCCCAGAGTCCCCGGCTGCTCACCAAGCTCCCTGGTCAGCTGCTCCCTGGCCATGGG | |
| CTGCAGCCACCACAGGGGCCTCCGGGTGGGCAAGCCGGAGGTCTTCGCCTGACCCCTGGG | |
| GGTATGGCACTACCTGGACAGCCTGGTGGCCCCTTCCTTAATACAGCTCTGGCCCAACAG | |
| CAGCAACAGCAACATTCTGGTGGGGCTGGATCCCTGGCTGGCCCTTCAGGGGGCTTCTTC | |
| CCTGGCAACCTTGCTCTTCGAAGCCTCGGACCTGATTCAAGGCTTTTACAGGAAAGGCAG | |
| CTGCAGCTGCAGCAGCAACGTATGCAGCTGGCCCAGAAACTGCAGCAGCAGCAGCAGCAG | |
| CAACAGCAGCAGCAGCACCTTCTAGGACAGGTGGCAATCCAGCAGCAACAGCAGCAGGGT | |
| CCTGGAGTACAGACAAACCAAGCTCTGGGTCCCAAGCCCCAGGGCCTTATGCCTCCCAGC | |
| AGCCACCAAGGCCTCCTGGTCCAGCAGCTGTCCCCTCAACCACCCCAGGGGCCCCAGGGC | |
| ATGCTGGGCCCTGCCCAGGTGGCTGTGTTGCAGCAGCAGCACCCTGGAGCTTTGGGCCCC | |
| CAGGGCCCTCACAGACAGGTGCTTATGACCCAGTCCCGGGTGCTCAGTTCCCCCCAGCTG | |
| GCACAGCAGGGTCAGGGCCTTATGGGACACAGGCTGGTCACAGCCCAGCAGCACCAGCAG | |
| CAACAACAGCACCAACAGCAAGGGTCCATGGCAGGGCTGTCCCATCTTCAGCAAAGTCTG | |
| ATGTCACACAGTGGGCAGCCCAAACTGAGCGCTCAGCCCATGGGCTCTTTACAGCAGCTT | |
| CAGCAGCAGCAGCAGCTGCAACAGCAACAGCAACTTCAGCAGCAGCAGCAGCAGCAGCTA | |
| CAACAGCAACAGCAACTTCAGCAGCAACAGCTTCAACAGCAGCAACAGCAGCAGCAGCTT | |
| CAACAACAGCAGCAGCAACAGCTTCAACAGCAGCAACAGCAGCTACAACAGCAACAGCAA | |
| CAACAACAGCAGCAGTTTCAACAGCAGCAGCAACAGCAGCAGATGGGCCTTTTAAACCAG | |
| AGTCGAACTTTACTGTCCCCTCAGCAACAACAGCAGCAGCAAGTGGCACTTGGCCCTGGC | |
| ATGCCAGCAAAGCCTCTTCAACACTTTTCTAGCCCTGGAGCCCTGGGTCCAACCCTCCTC | |
| CTGACGGGCAAGGAACAAAACACCGTAGACCCAGCCGTTTCTTCAGAGGCCACTGAGGGG | |
| CCCTCTACACATCAGGGAGGGCCGTTACCAATAGGAACTACCCCTGAGTCAATGGCCACT | |
| GAACCAGGAGAGGTAAAGCCCTCACTCTCTGGGGACTCACAACTCCTGCTTGTCCAACCC | |
| CAGCCCCAGCCTCAGCCCAGCTCTCTGCAGCTGCAGCCACCTCTGAGGCTTCCAGGACAA | |
| CAGCAGCAGCAAGTTAGCCTGCTCCACACAGCAGGTGGAGGAAGCCATGGGCAGCTAGGC | |
| AGTGGATCATCTTCTGAGGCCTCATCTGTGCCCCACCTGCTGGCTCAGCCCTCTGTTTCC | |
| TTAGGGGATCAGCCTGGGTCCATGACCCAGAACCTTCTGGGCCCCCAACAGCCCATGCTA | |
| GAGCGGCCCATGCAAAATAATACAGGGCCACAACCTCCCAAACCAGGACCTGTCCTCCAG | |
| TCTGGGCAGGGTCTGCCTGGGGTTGGAATCATGCCTACGGTGGGTCAGCTTCGAGCACAG | |
| CTCCAAGGAGTCCTGGCCAAAAACCCACAGCTGCGGCACTTAAGTCCTCAGCAGCAGCAG | |
| CAGCTACAGGCACTCCTCATGCAGCGGCAGCTGCAGCAGAGTCAGGCAGTACGCCAGACC | |
| CCACCCTACCAGGAGCCTGGGACCCAGACCTCTCCCCTCCAGGGCCTCCTGGGCTGCCAA | |
| CCTCAACTTGGGGGCTTCCCTGGACCACAGACAGGCCCCCTCCAGGAGCTAGGGGCAGGG | |
| CCTCGACCTCAGGGCCCACCCCGGCTCCCTGCCCCACCAGGAGCCTTATCTACAGGACCA | |
| GTCCTTGGCCCTGTCCATCCCACACCTCCACCATCCAGCCCTCAAGAGCCAAAGAGACCT | |
| TCACAATTACCTTCCCCCAGCTCCCAGCTTCCCACTGAGGCCCAGCTCCCTCCCACCCAT | |
| CCAGGGACCCCCAAACCTCAGGGGCCAACCTTGGAGCCGCCTCCTGGGAGGGTCTCACCT | |
| GCTGCTGCCCAGCTTGCAGATACCTTGTTTAGCAAGGGTCTGGGACCTTGGGATCCCCCA | |
| GACAACCTAGCAGAAACCCAGAAGCCAGAGCAGAGCAGCCTGGTACCTGGGCATCTGGAC | |
| CAGGTGAATGGACAGGTGGTGCCTGAGGCATCCCAACTCAGCATCAAGCAGGAACCTCGG | |
| GAAGAGCCATGTGCCCTGGGAGCCCAGTCAGTGAAGAGGGAGGCCAATGGGGAGCCAATA | |
| GGGGCACCAGGAACCAGCAACCACCTCCTGCTGGCAGGCCCTCGCTCAGAAGCTGGGCAT | |
| CTGCTCTTGCAGAAGCTACTCCGGGCAAAGAATGTGCAACTCAGCACTGGGCAGGGGTCC | |
| GAGGGGCTGCGAGCTGAGATCAACGGGCACATTGACAGCAAGCTGGCTGGGCTGGAGCAG | |
| AAACTACAGGGTACCCCCAGCAACAAGGAGGATGCAGCAGCAAGGAAGCCTTTGACACCG | |
| AAGCCCAAGCGGGTACAGAAGGCAAGCGACAGGTTGGTGAGCTCCCGAAAGAAGCTGCGG | |
| AAGGAGGACGGCGTCAGGGCCAGCGAGGCCTTGCTGAAACAGCTGAAACAGGAGCTGTCC | |
| CTGCTGCCCCTAACGGAGCCTGCTATCACCGCCAATTTTAGCCTCTTTCCCCCCTTTGGC | |
| AGTGGCTGCCCAGTCAATGGGCAGAGCCAGCTGAGGGGGGCCTTTGGAAGTGGGCCGCTG | |
| CCCACTGGCCCTGACTACTATTCCCAGCTGCTTACCAAGAATAACCTGAGTAACCCGCCG | |
| ACACCACCCTCGTCGCTGCCCCCCACCCCACCCCCATCGGTGCAGCAGAAGATGGTGAAT | |
| GGCGTCACCCCATCTGAAGAGCTGGGGGAGCACCCCAAGGATGCTGCCTCTGCCCGGGAT | |
| AGTGAAAGGGCACTGAGGGATACTTCAGAGGTGAAGAGTCTAGACCTGCTGGCTGCCTTG | |
| CCTACACCCCCTCACAATCAGACTGAGGATGTCAGGATGGAGAGTGATGAGGATAGCGAT | |
| TCTCCTGACAGCATTGTGCCAGCTTCATCCCCTGAGAGCATCTTGGGGGAGGAGGCCCCT | |
| CGTTTCCCTCATCTGGGCTCAGGCCGGTGGGAGCAAGAGGACCGGGCCCTCTCCCCTGTC | |
| ATCCCCCTCATTCCTCGGGACAGCATCCCAGTCTTCCCAGATACCAAACCTTATGGGGCC | |
| CTTGGCCTGGAGGTCCCTGGAAAGCTGCCTGTCACAACTTGGGAAAAGGGCAAAGGAAGT | |
| GAGGTGTCAGTCATGCTCACAGTCTCTGCTGCTGCAGACAAGAACCTGAATGGCGTGATG | |
| GTGGCAGTGGCGGAGCTGCTGAGCATGAAGATCCCCAACTCCTATGAGGTGCTGTTCCCA | |
| GAGAGCCCCGCCCGGGGAGGCACTGAGCCAAAGAAGGGGGAAGCTGAGGGTCCTGGTGGG | |
| AAGGAAAAGGGTCTGGAAGGCAAGAGCCCAGACACTGGCCCTGATTGGCTGAAGCAGTTT | |
| GATGCAGTGTTGGCTGGCTATACCCTGAAGAGGCAACTAGACATCTTGAGCCTCCTGAAA | |
| CAGGAGAGCCCCGCCCCAGAGCCACCCACTCAGCACAGGTATACCTACAATGTCTCCAAT | |
| CTGGATGTGCGACACCTCTCGGCCCCACCTCCTGAAGAACCCTCCCCGCCCCCTTCCCCC | |
| TTGGCACCTTCTCCTGCCAGTCCCCCTACTGAGCCCTTGGTTGAACTTCCCACCGAACCC | |
| TTGGCTGAGCCACCCGTCCCCTCACCTCTGCCACTGGCCTCATCCCCTGAATCAGCCCGA | |
| CCCAAGCCCCGTGCCCGGCCCCCTGAAGAAGGTGAAGATACCCGTCCTCCTCGCCTCAAG | |
| AAATGGAAAGGAGTGCGCTGGAAGCGGCGCTTACGAGGTGCCATGTTGGAGCTTTTTGGT | |
| GTGAACAGTCTGCAACTAAAATTTAGGACCAGAAGCGAGAATGGCGTTTTAATCCATATC | |
| CAAGAAAGCAGCAATTACACTACTGTGAAGATTAAGAATGGCAAAGTATATTTTACATCC | |
| GATGCAGGAATTGCTGGGAAAGTGGAGAGAAATATTCCTGAAGTATATGTTGCAGACGGC | |
| CACTGGCACACTTTTCTAATTGGGAAAAATGGAACAGCAACAGTATTGTCTGTTGACAGA | |
| ATATATAACAGAGATATTATCCACCCTACTCAGGACTTCGGTGGCCTTGATGTGCTTACT | |
| ATATCACTTGGAGGAATTCCACCCAATCAAGCACATCGAGATGCCCAAACAGCAGGTTTT | |
| GATGGCTGCATTGCTTCTATGTGGTATGGTGGAGAAAGTCTTCCTTTCAGCGGGAAGCAT | |
| AGCTTGGCCTCCATCTCAAAAACAGATCCCTCAGTGAAGATTGGCTGCCGTGGCCCGAAC | |
| ATTTGTGCCAGCAACCCCTGCTGGGGTGATTTGCTGTGCATTAATCAGTGGTATCCCTAC | |
| AGGTGTGTCCCTCCTGGGGACTGTGCCTCCCACCCGTGCCAGAATGGTGGCAGCTGTGAG | |
| CCAGGCCTGCACTCCGGCTTCACCTGTAGCTGCCCAGACTCGCACACGGGAAGGACCTGT | |
| GAGATGGTGGTGGCCTGTCTTGGCGTCCTCTGTCCTCAGGGGAAGGTGTGCAAAGCTGGA | |
| AGTCCTGCGGGGCATGTCTGTGTTCTGAGTCAGGGCCCTGAAGAGATCTCTCTGCCTTTG | |
| TGGGCTGTGCCTGCCATCGTGGGCAGCTGCGCAACCGTCTTGGCCCTCCTGGTCCTTAGC | |
| CTGATCCTGTGTAACCAGTGCAGGGGGAAGAAGGCCAAAAATCCCAAAGAGGAGAAGAAA | |
| CCGAAGGAGAAGAAGAAAAAGGGAAGTGAGAACGTTGCTTTTGATGACCCTGACAATATC | |
| CCTCCCTATGGGGATGACATGACTGTGAGGAAGCAGCCTGAAGGGAACCCAAAACCAGAT | |
| ATCATTGAAAGGGAAAACCCCTACCTTATCTATGATGAAACTGATATTCCTCACAACTCA | |
| GAAACCATCCCCAGCGCCCCTTTGGCATCTCCAGAGCAGGAGATAGAGCACTATGACATT | |
| GACAACGCCAGCAGCATCGCCCCTTCGGATGCAGACATCATTCAACACTACAAGCAGTTC | |
| CGCAGCCACACACCAAAATTTTCAATCCAGAGGCACAGTCCCCTAGGCTTTGCAAGGCAA | |
| TCCCCCATGCCCTTAGGAGCAAGCAGTTTGACTTACCAGCCTTCATATGGTCAAGGTTTG | |
| AGAACCAGCTCCCTAAGCCACTCAGCATGCCCAACTCCCAACCCTCTGTCTCGACACAGT | |
| CCAGCCCCTTTCTCCAAATCTTCTACGTTCTATAGAAACAGCCCAGCAAGGGAATTGCAT | |
| CTTCCTATAAGGGATGGTAATACTTTGGAAATGCATGGTGACACCTGCCAACCTGGCATT | |
| TTCAACTATGCCACAAGGCTGGGAAGGAGAAGCAAGAGTCCTCAGGCCATGGCATCACAT | |
| GGTTCTAGACCAGGGAGTCGCCTAAAGCAGCCGATTGGGCAGATTCCACTGGAATCTTCT | |
| CCTCCAGTCGGACTTTCTATTGAAGAAGTGGAGAGGCTCAACACACCTCGCCCTAGAAAC | |
| CCAAGTATCTGCAGTGCAGACCATGGGAGGTCTTCTTCAGAGGAGGACTGCAGAAGGCCA | |
| CTGTCTAGAACAAGGAATCCAGCGGATGGCATTCCAGCTCCAGAATCCTCTTCTGATAGT | |
| GACTCCCATGAATCTTTCACTTGCTCAGAAATGGAATATGACAGGGAGAAGCCAATGGTA | |
| TATACTTCCAGAATGCCCAAATTATCTCAAGTCAATGAATCTGATGCAGATGATGAAGAT | |
| AATTATGGAGCCAGACTCAAGCCTCGAAGGTACCACGGTCGCAGGGCCGAGGGAGGACCT | |
| GTGGGCACCCAGGCAGCAGCACCAGGCACTGCTGACAACACACTGCCCATGAAGCTAGGG | |
| CAGCAAGCAGGGACTTTCAACTGGGACAACCTTTTGAACTGGGGCCCTGGCTTTGGCCAT | |
| TATGTAGATGTTTTTAAAGATTTGGCATCTCTTCCAGAAAAAGCAGCAGCAAATGAAGAA | |
| GGCAAAGCTGGGACAACTAAACCAGTCCCCAAAGATGGGGAAGCAGAACAGTATGTGTGA | |
| AGTTTATGTACTGGCACTATAAAATATAAAAACAAGAAATAATACTTCAAACCATTGTAA | |
| AGTTGCTGACTAGGTTGGGGTCACATTTGAAAAACAGGGCCAGTATGGGACTAGTGGGTG | |
| GGAGGGGAAAACTTTTAAAATTATTAACCACATTGCTGGCTGAAA | |

In a search of public sequence databases, the NOV47 nucleic acid sequence, located on chromsome 12q 12.14 has 14307 of 14312 bases (99%) identical to a gb:GENBANK-ID:AF010404|acc:AF010404.1 mRNA from Homo sapiens (Homo sapiens ALR mRNA, complete cds). Public nucleotide databases include all GenBank databases and the GeneSeq patent database.

The disclosed NOV47 polypeptide (SEQ ID NO:112) encoded by SEQ ID NO:111 has 5159 amino acid residues and is presented in Table 47B using the one-letter amino acid code. Signal P, Psort and/or Hydropathy results predict that NOV47 has no signal peptide and is likely to be localized in the cytoplasm with a certainty of 0.9800.

TABLE 47B
Encoded NOV47 protein sequence.

| | |
|---|---|
| MSPPPEESPMSPPPEASRLFPPFEESPLSPPPEESPLSPPPEASRLSPPPEDSPMSPPPE | (SEQ ID NO:112) |
| ESPMSPPPEVSRLSPLPVVSRLSPPPEESPLSPPPEESPTSPPPEASRLSPPPEDSPTSP | |
| PPEDSPASPPPEDSLMSLPLEESPLLPLPEEPQLCPRSEGPHLSPRPEEPHLSPRPEEPH | |
| LSPQAEEPHLSPQPEEPCLCAVPEEPHLSPQAEGPHLSPQPEELHLSPQTEEPHLSPVPE | |
| EPCLSPQPEESHLSPQSEEPCLSPRPEESHLSPELEKPPLSPRPEKPPEEPGQCPAPEEL | |
| PLFPPPGEPSLSPLLGEPALSEPGEPPLSPLPEELPLSPSGEPSLSPQLMPPDPLPPPLS | |
| PIITAAAPPALSPLGELEYPFGAKGDSDPESPLAAPILETPISPPPEANCTDPEPVPPMI | |
| LPPSPGSPVGPASPILMEPLPPQCSPLLQHSLVPQNSPPSQCSPPALPLSVPSPLSPIGK | |
| VVGVSDEAELHEMETEKVSEPECPALEPSATSPLPSPMGDLSCPAPSPAPALDDFSGLGE | |
| DTAPLDGIDAPGSQPEPGQTPGSLASELKGSPVLLDPEELAPVTPMEVYPECKQTAGRGS | |
| PCEEQEEPRAPVAPTPPTLIKSDIVNEISNLSQGDASASFPGSEPLLGSPDPEGGGSLSM | |
| ELGVSTDVSPARDECSLRLCTDSLPETDDSLLCDAGTAISGGKAEGEKGRRRSSPARSRI | |
| KQGRSSSFPGRRRPRGGAHGGRGRGRALRKSTASSIETLVVADIDSSPSKEEEEEDDDTM | |
| QNTVVLFSNTDKFVLMQDMCVVCGSFGRGAEGHLLACSQCSQCYHPYCVNSKITKVMLLK | |
| GWRCVECIVCEVCGQASDPSRLLLCDDCDISYHTYCLDPPLLTVPKGGWKCKWCVSCMQC | |
| GAASPGFHCEWQNSYTHCGPCASLVTCPICHAPYVEEDLLIQCRHCERWMHAGCESLFTE | |
| DDVDHAPDEGFDCVSCQPYVVKPVAPVAPPELVPMKVKEPEPQYFRFEGVWLTETGMALL | |
| RNLTMSPLHKRRQRRGRLGLPGEAGLEGSEPSDALGPDDKKDGDLDTDELLKGEGGVEHM | |
| ECEIKLEGPVSPDVEPGKEETEESKKRKRKPYRPGIGGFMVRQRKSHTRTKKGPAAQAEV | |
| LSGDGQPDEVIPADLPAEGAVEQSLAEGDEKKKQQRRGRKRSKLEGMFPAYLQEAFFGKE | |
| LLDLSRKALFAVGVGRPSFGLGTPKAKGDGGSERKELPTSQKGDDGPDIADEESRGLEGK | |
| ADTPGPEDGGVKASPVPSDPEKPGTPGEGMLSSDLDRISTEELPKMESKDLQQLFKDVLG | |
| SEREQHLGCGTPGLEGSRTPLQRPFLQGGLPLGNLPSSSPMDSYPGLCQSPFLDSRERGG | |
| FFSPEPGEPDSPWTGSGGTTPSTPTTPTTEGEGDGLSYNQRSLQRWEKDEELGQLSTISP | |
| VLYANINFPNLKQDYPDWSSRCKQIMKLWRKVPAADKAPYLQKAKDNRAAHRINKVQKQA | |
| ESQINKQTKVGDIARKTDRPALHLRIPPQPGALGSPPPAAAPTIFIGSPTTPAGLSTSAD | |
| GFLKPPAGSVPGPDSPGELFLKLPPQVPAQAPSQDPFGLAPAYPLEPRFPTAPPTYPPYP | |
| SPTGAPAQPPMLGASSRPGAGQPGEFHTTPPGTPRHQPSTPDPFLKPRCPSLDNLAVPES | |
| PGVGGGKASEPLLSPPPFGESRKALEVKKEELGASSPSYGPPNLGFVDSPSSGTHLGGLE | |
| LKTPDVFKAPLTPRASQVEPQSPGLGLRPQEPPPAQALAPSPPSHPDIFRPGSYTDPYAQ | |
| PPLTPRPQPPPPESCCALPPRSLPSDPFSRVPVSPQSQSSSQSPLTPRPLSAEAFCPSPV | |
| TPRFQSPDPYSRPPSRPQSRDPFAPLHKPPRPQPFEVAFKAGSLAHTSLGAGGFPAALPA | |
| GPAGELHAKVPSGQPPNFVRSPGTGAFVGTPSPMRFTFPQAVGEPSLKPPVPQPGLPPPH | |
| GINSHFGPGPTLGKPQSTNYTVATGNFHPSGSPLGPSSGSTGESYGLSPLRPPSVLPPPA | |
| PDGSLPYLSHGASQRSGITSPVEKREDPGTGMGSSLATAELPGTQDPGMSGLSQTELEKQ | |
| RQRQRLRELLIRQQIQRNTLRQEKETAAAAAGAVGPPGSWGAEPSSPAFEQLSRGQTPFA | |
| GTQDKSSLVGLPPSKLSGPILGPGSFPSDDRLSRPPPPATPSSMDVNSRQLVGGSQAFYQ | |
| RAPYPGSLPLQQQQQQLWQQQQATAATSMRFAMSARFPSTPGPELGRQALGSPLAGISTR | |
| LPGPGEPVPGPAGPAQFIELRHNVQKGLGPGGTPFPGQGPPQRPRFYPVSEDPHRLAPEG | |
| LRGLAVSGLPPQKPSAPPAPELNNSLHPTPHTKGPTLPTGLELVNRPPSSTELGRPNPLA | |
| LEAGKLPCEDPELDDDFDAHKALEDDEELAHLGLGVDVAKGDDELGTLENLETNDPHLDD | |
| LLNGDEFDLLAYTDPELDTGDKKDIFNEHLRLVESANEEAEREALLRGVEPGPLGPEERP | |
| PPAADASEPRLASVLPEVKPKVEEGGRHPSPCQFTIATPKVEPAPAANSLGLGLKPGQSM | |
| MGSRDTRMGTGPFSSSGHTAEKASFCATGGPPAHLLTPSPLSGPGGSSLLEKFELESGAL | |
| TLPGGPAASGDELDKMESSLVASELPLLIEDLLEHEKKELQKKQQLSAQLQPAQQQQQQQ | |
| QQHSLLPAPGPAQAMSLPHEGSSPSLAGSQQQLSLGLAVARQPGLPQPLMPTQPPAHALQ | |
| QRLAPSMAMVSNQGHMLSGQHGGQAGLVPQQSSQPVLSQKPMGTMPPSMCMKPQQLAMQQ | |
| QLANSFFPDTDLDKFAAEDIIGPIAKAKMVALKGIKKVMAQGSIGVAPGMNRQQVSLLAQ | |
| RLSGGPSSDLQNHVAAGSGQERSAGDPSQPRPNPPTFAQGVINEADQRQYEEWLFHTQQL | |
| LQMQLKVLEEQIGVHRKSRKALCAKQRTAKKAGREFPEADAEKLKLVTEQQSKIQKQLDQ | |
| VRKQQKEHTNLMAEYRNKQQQQQQQQQQQQQQHSAVLALSPSQSPRLLTKLPGQLLPGHG | |
| LQPPQGPPGGQAGGLRLTPGGMALPGQPGGPFLNTALAQQQQQQHSGGAGSLAGPSGGFF | |
| PGNLALRSLGPDSRLLQERQLQLQQQRMQLAQKLQQQQQQQQQQQHLLGQVAIQQQQQQG | |
| PGVQTNQALGPKPQGLMPPSSHQGLLVQQLSFQPPQGPQGMLGPAQVAVLQQQHPGALGP | |
| QGPHRQVLMTQSRVLSSPQLAQQGQGLMGHRLVTAQQQQQQQQHQQQGSMAGLSHLQQSL | |
| MSHSGQPKLSAQPMGSLQQLQQQQQLQQQQQLQQQQQQQLQQQQQLQQQQLQQQQQQQQL | |
| QQQQQQQLQQQQQQLQQQQQQQQQQFQQQQQQQQMGLLNQSRTLLSPQQQQQQQVALGPG | |
| MPAKPLQHFSSPGALGPTLLLTGKEQNTVDPAVSSEATEGPSTHQGGPLAIGTTPESMAT | |
| EPGEVKPSLSGDSQLLLVQPQPQPQPSSLQLQPPLRLPGQQQQQVSLLHTAGGGSHGQLG | |
| SGSSSEASSVPHLLAQPSVSLGDQPGSMTQNLLGPQQPMLERPMQNNTGPQPPKPGPVLQ | |
| SGQGLPGVGIMPTVGQLRAQLQGVLAKNPQLRHLSPQQQQQLQALLMQRQLQQSQAVRQT | |
| PPYQEPGTQTSPLQGLLGCQFQLGGFPGPQTGPLQELGAGPRPQGPPRLPAPPGALSTGP | |
| VLGPVHPTPPPSSPQEPKRPSQLPSPSSQLPTEAQLPPTHPGTPKPQGPTLEPPPGRVSP | |
| AAAQLADTLFSKGLGPWDPPDNLAETQKPEQSSLVPGHLDQVNGQVVPEASQLSIKQEPR | |
| EEPCALGAQSVKREANGEPIGAPGTSNHLLLAGPRSEAGHLLLQKLLRAKNVQLSTGQGS | |
| EGLRAEINGHIDSKLAGLEQKLQGTPSNKEDAAARKPLTPKPKRVQKASDRLVSSRKKLR | |
| KEDGVRASEALLKQLKQELSLLPLTEPAITANFSLFAPFGSGCPVNGQSQLRGAFGSGAL | |
| PTGPDYYSQLLTKNNLSNPPTPPSSLPPTPPPSVQQKMVNGVTPSEELGEHPKDAASARD | |
| SERALRDTSEVKSLDLLAALPTPPHNQTEDVRMESDEDSDSPDSIVPASSPESILGEEAP | |
| RFPHLGSGRWEQEDRALSPVIPLIPRDSIPVFPDTKPYGALGLEVPGKLPVTTWEKGKGS | |
| EVSVMLTVSAAADKNLNGVMVAVAELLSMKIPNSYEVLFPESPARGGTEPKKGEAEGPGG | |
| KEKGLEGKSPDTGPDWLKQFDAVLAGYTLKRQLDILSLLKQESPAPEPPTQHRYTYNVSN | |
| LDVRQLSAPPPEEPSPPPSPLAPSPASPPTEPLVELPTEPLAEPPVPSPLPLASSPESAR | |
| PKPRARPPEEGEDTRPPRLKKWKGVRWKRRLRGAMLELFGVNSLEVKFRTRSENGVLIHI | |
| QESSNYTTVKIKNGKVYFTSDAGIAGKVERNIPEVYVADGHWHTFLIGKNGTATVLSVDR | |
| IYNRDIIHPTQDFGGLDVLTISLGGIPPNQAHRDAQTAGFDGCIASMWYGGESLPFSGKH | |
| SLASISKTDPSVKIGCRGPNICASNPCWGDLLCINQWYAYRCVPPGDCASHPCQNGGSCE | |
| PGLHSGFTCSCPDSHTGRTCEMVVACLGVLCPQGKVCKAGSPAGHVCVLSQGPEEISLPL | |
| WAVPAIVGSCATVLALLVLSLILCNQCRGKKAKNPKEEKKPKEKKKKGSENVAFDDPDNI | |
| PPYGDDMTVRKQPEGNPKPDIIERENPYLIYDETDIPHNSETIPSAPLASPEQEIEHYDI | |
| DNASSIAPSDADIIQHYKQFRSHTPKFSIQRHSPLGFARQSPMPLGASSLTYQPSYGQGL | |
| RTSSLSHSACPTPNPLSRHSPAPFSKSSTFYRNSPARELHLPIRDGNTLEMHGDTCQPGI | |
| FNYATRLGRRSKSPQAMASHGSRPGSRLKQPIGQIPLESSPPVGLSIEEVERLNTPRPRN | |
| PSICSADHGRSSSEEDCRRPLSRTRNPADGIPAPESSSDSDSHESFTCSEMEYDREKPMV | |
| YTSRMPKLSQVNESDADDEDNYGALRKPRRYHGRRAEGGPVGTQAAAPGTADNTLPMKLG | |
| QQAGTFNWDNLLNWGPGFGHYVDVFKDLASLPEKAAANEEGKAGTTKPVPKDGEAEQYV | |

A search of sequence databases reveals that the NOV47 amino acid sequence has 4776 of 4796 amino acid residues (99%) identical to, and 4779 of 4796 amino acid residues (99%) similar to, the 4957 amino acid residue ptnr:SPTR-EMBL-ACC:O14687 protein from Homo sapiens (Human) (ALR). Public amino acid databases include the GenBank databases, SwissProt, PDB and PIR.

NOV47 is expressed in at least brain. This information was derived by determining the tissue sources of the sequences that were included in the invention including but not limited to SeqCalling sources, Public EST sources, Literature sources, and/or RACE Sources.

The disclosed NOV47 polypeptide has homology to the amino acid sequences shown in the BLASTP data listed in Table 47C.

TABLE 47C
BLAST results for NOV47

| Gene Index/ | | Length | Identity | Positives | |
|---|---|---|---|---|---|
| Identifier | Protein/Organism | (aa) | (%) | (%) | Expect |
| gi\|7512280\|pir\|\|T03455 | ALR protein - | 4957 | 4384/4427 | 4385/4427 | 0.0 |
| | human | | (99%) | (99%) | |
| gi\|18601907\|ref\|XP_ | myeloid/lymphoid | 3492 | 2930/2944 | 2933/2944 | 0.0 |
| 028760.2\| | or mixed-lineage | | (99%) | (99%) | |
| (XM_028760) | leukemia 2 | | | | |
| | [Homo sapiens] | | | | |
| gi\|3540281\|gb\|AAC34383.1\| | All-1 related | 4823 | 648/1458 | 822/1458 | 0.0 |
| (AF056116) | protein [Takifugu | | (44%) | (55%) | |
| | rubripes] | | | | |
| gi\|13640139\|ref\|XP_ | protein FLJ23056 | 317 | 315/317 | 315/317 | e−149 |
| 017017.1\| | [Homo sapiens] | | (99%) | (99%) | |
| (XM_017017) | | | | | |
| gi\|10864041\|ref\|NP_ | myeloid/lymphoid | 4025 | 340/802 | 467/802 | e−135 |
| 067053.1\| | or mixed-lineage | | (42%) | (57%) | |
| (NM_021230) | leukemia 3; ALR- | | | | |
| | like protein | | | | |
| | [Homo sapiens] | | | | |

Tables 47D-H list the domain descriptions from DOMAIN analysis results against NOV47. This indicates that the NOV47 sequence has properties similar to those of other proteins known to contain this domain.

TABLE 47D
Domain Analysis of NOV47

gnl|Smart|smart00282, LamG, Laminin G domain
CD-Length = 135 residues, 98.5% aligned
Score = 85.1 bits (209), Expect = 9e−17

TABLE 47E
Domain Analysis of NOV47

gnl|Pfam|pfam00628, PHD, PHD-finger. PHD folds into an interleaved
type of Zn-finger chelating 2 Zn ions in a similar manner to that of
the RING and FYVE domains.
CD-Length = 49 residues, 98.0% aligned
Score = 71.2 bits (173), Expect = 1e−12

TABLE 47F
Domain Analysis of NOV47

gnl|Pfam|pfam00769, ERM, Ezrin/radixin/moesin family. This family of
proteins contain a band 4.1 domain (pfam00373), at their amino
terminus. This family represents the rest of these proteins.
CD-Length = 365 residues, 23.0% aligned
Score = 52.8 bits (125), Expect = 5e−07

TABLE 47G
Domain Analysis of NOV47

gnl|Pfam|pfam00529, HlyD, HlyD family secretion protein.
CD-Length = 310 residues, 53.9% aligned
Score = 53.9 bits (128), Expect = 2e−07

TABLE 47H
Domain Analysis of NOV47

gnl|Pfam|pfam02166, Androgen_recep, Androgen receptor.
CD-Length = 456 residues, 18.6% aligned
Score = 52.8 bits (125), Expect = 5e−07

The ALL-1 gene is involved in human acute leukemia through chromosome translocations or internal rearrangements. ALL-1 is the human homologue of Drosophila trithorax. The latter is a member of the trithorax group (trx-G) genes which together with the Polycomb group (Pc-G) genes act as positive and negative regulators, respectively, to determine the body structure of Drosophila. ALR encodes a gigantic 5262 amino acid long protein containing a SET domain, five PHD fingers, potential zinc fingers, and a very long run of glutamines interrupted by hydrophobic residues, mostly leucine. The SET motif, PDH fingers, zinc fingers and two other regions are most similar to domains of ALL-1 and TRX. The first two motifs are also found in other trx-G and Pc-G proteins. The ALR gene was mapped to chromosome band 12q12-13, adjacent to the VDR gene. This region is involved in duplications and translocations associated with cancer.

The human ALL-1/MLL/HRX gene on chromosome 11q23 is the site of many locally clustered chromosomal alterations associated with several types of acute leukemias, including deletions. partial duplications and reciprocal translocations. Structurally variant proteins derived from an altered ALL-1 gene presumably make essential contributions to the malignant transformation of hematopoietic progenitor cells.

Many haematologic malignancies carry characteristic chromosomal translocations, which are thought to play an important role in the pathogenesis of these tumours. The t(8; 14) translocation in Burkitt's lymphoma was one of the first characterized at the molecular level. In this translocation the c-myc oncogene at chromosome 8q24 becomes deregulated by enhancer elements of the immunoglobulin heavy chain locus at chromosome 14q32 leading to a very aggressive B cell malignancy. Chromosomal translocations involving the MLL gene occur in about 80% of infant leukemias.

The disclosed NOV47 nucleic acid of the invention encoding a ALR-like protein includes the nucleic acid whose sequence is provided in Table 47A or a fragment thereof. The invention also includes a mutant or variant nucleic acid any of whose bases may be changed from the corresponding base shown in Table 47A while still encoding a protein that maintains its ALR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV47 protein of the invention includes the ALR-like protein whose sequence is provided in Table 47B. The invention also includes a mutant or variant protein any of whose residues may be changed from the corresponding residue shown in Table 47B while still encoding a protein that maintains its ALR-like activities and physiological functions, or a functional fragment thereof. In the mutant or variant protein, up to about 1 percent of the residues may be so changed.

The invention further encompasses antibodies and antibody fragments, such as F_{ab} or (F_{ab})₂, that bind immunospecifically to any of the proteins of the invention.

The above defined information for this invention suggests that this ALR-like protein (NOV47) may function as a member of a “ALR family”. Therefore, the NOV47 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV47 nucleic acids and proteins of the invention are useful in potential therapeutic applications implicated in diseases including but not limited to various pathologies and disorders as indicated below. For example, a cDNA encoding the ALR-like protein (NOV47) may be useful in gene therapy, and the ALR-like protein (NOV47) may be useful when administered to a subject in need thereof. By way of nonlimiting example, the compositions of the present invention will have efficacy for treatment of patients suffering from Osteoporosis, involutional; Rickets, vitamin D-resistant; Fibrosis of extraocular muscles, congenital, I; Achalasia-addisonianism-alacrimia syndrome; Cataract, polymorphic and lamellar; acute leukemias, cancers, or other pathologies or conditions. The NOV47 nucleic acid encoding the ALR-like protein of the invention, or fragments thereof, may further be useful in diagnostic applications, wherein the presence or amount of the nucleic acid or the protein are to be assessed.

NOV47 nucleic acids and polypeptides are further useful in the generation of antibodies that bind immuno-specifically to the novel NOV47 substances for use in therapeutic or diagnostic methods. These antibodies may be generated according to methods known in the art, using prediction from hydrophobicity charts, as described in the “Anti-NOVX Antibodies” section below. The disclosed NOV47 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV48

A disclosed NOV48 nucleic acid of 1988 nucleotides (also referred to as CG57713-01) encoding a sodium/bile acid transporter-like protein is shown in Table 48A. The start and stop codons are in bold letters.

TABLE 48A
NOV48 nucleotide sequence.

| | |
|---|---|
| GGCGGCGACTGCGGCGACCGCGGGACGGCGAGAGGCACGCGGCGGGAGGGGACCGGAATC | (SEQ ID NO:113) |
| CGCAGCTCCGGCCGCGCCATGGACGGCAACGACAACGTGACCCTGCTCTTCGCCCCTCTG | |
| CTGCGGGACAACTACACCCTGGCGCCCAATGCCAGCAGCCTGGGCCCCGGCACGGACCTC | |
| GCCCTCGCCCCTGCCTCCAGCGCCGGCCCCGGCCCTGGGCTCAGCCTCGGGCCGGGTCCG | |
| AGCTTCGGCTTCAGCCCCGGCCCCACTCCGACCCCGGAGCCCACGACCAGCGGCCTCGCG | |
| GGCGGCGCGGCGAGCCACGGCCCTTCCCCGTTCCCTCGGCCCTGGGCGCCCCACGCGCTC | |
| CCGTTCTGGGACACGCCGCTGAACCACGGGCTGAACGTGTTCGTGGGCGCCGCCCTGTGC | |
| ATCACCATGCTGGGCCTGGGCTGCACGGTGGACGTGAACCACTTCGGGGCGCACGTCCGT | |
| CGGCCCGTGGGCGCGCTGCTGGCAGCGCTCTGCCAGTTCGGCCTCCTGCCGCTGCTGGCC | |
| TTCCTGCTGGCCCTCGCCTTCAAGCTGGACGAGGTGGCCGCCGTGGCGGTGCTCCTGTGT | |
| GGCTGCTGTCCCGGCGGCAATCTCTCCAATCTTATGTCCCTGCTGGTTGACGGCGACATG | |
| AACCTCAGCATCATCATGACCATCTCCTCCACGCTTCTGGCCCTCGTCTTGATGCCCCTG | |
| TGCCTGTGGATCTACAGCTGGGCTTGGATCAACACCCCTATCGTGCAGTTACTACCCCTA | |
| GGGACCGTGACCCTGACTCTCTGCAGCACTCTCATACCTATCGGGTTGGGCGTCTTCATT | |
| CGCTACAAATACAGCCGGGTGGCTGACTACATTGTGAAGGTAAGGCCCGTTTCCCTGTGG | |
| TCTCTGCTAGTGACTCTGGTGGTCCTTTTCATAATGACCGGCACTATGTTAGGACCTGAA | |
| CTGCTGGCAAGTATCCCTGCAGCTGTTTATGTGATAGCAATTTTTATGCCTTTGGCAGGC | |
| TACGCTTCAGGTTATGGTTTAGCTACTCTCTTCCATCTTCCACCCAACTGCAAGAGGACT | |
| GTATGTCTGGAAACAGGTAGTCAGAATGTGCAGCTCTGTACAGCCATTCTAAAACTGGCC | |
| TTTCCACCGCAATTCATAGGAAGCATGTACATGTTTCCTTTGCTGTATGCACTTTTCCAG | |
| TCTGCAGAAGCGGGGATTTTTGTTTTAATCTATAAAATGTATGGAAGTGAAATGTTGCAC | |
| AAGCGAGATCCTCTAGATGAAGATGAAGATACAGATATTTCTTATAAAAAACTAAAAGAA | |
| GAGGAAATGGCAGACACTTCCTATGGCACAGTGAAAGCAGAAAATATAATAATGATGGAA | |
| ACCGCTCAGACTTCTCTCTAAATGTGGAGATACACAGGAGCTTCTATCTTGCTGAAATAT | |
| TGCTTCATATTTATAGCCTGTCGTAGTGCACATGGTTAACATAAAAGATAACACTGGTTC | |
| ACATCATACATGTAACAATTCTGATCTTTTTAAGGTTCACTGGTGTATTAACCAAACGTT | |
| GTCACAAATTACAAATCAATGCTGTAATATAATTTGCACCTGGAATGGCTAACGTGAAGC | |
| CTGAATTAAATGTGGTTTTTAGTTTTTACCATCACCAATTTCTATGACTGTTGCAAATAC | |
| AGAATCTATTAGAAAACAGGGTCTTGGAAATGTAGAATTTTGGCGCACTATGAGGAAAAA | |
| CAAGCTATCTTTGTAAAGCATAATTGAGTTTAATGTAATTGTTGTAAAAAAAAAAGTGTG | |
| CTTGCTCTACTTAAAATTCCTCACAATGTTGAATTTTGACCTGTATTCAGAAGAATTCCA | |
| AAACAGGTCAGTTAAATAAGGAAATATAGTATTTGTCAAACCAGTATCAGAGAAAAGTTA | |
| CATTAATGTATTTGATTACTTGATCTGGTATCTACTTATTAATGAATAATCAACATTTTT | |
| CTAGTGAA | |

In a search of public sequence databases, the NOV48 nucleic acid sequence, located on chromsome 4 has 250 of 395 bases (63%) identical to a gb:GENBANK-ID:HUMNTCP|acc:L21893.1 mRNA from Homo sapiens (Human Na/taurocholate cotransporting polypeptide mRNA, complete cds). Public nucleotide databases include all GenBank databases and the GeneSeq patent database.

The disclosed NOV48 polypeptide (SEQ ID NO:114) encoded by SEQ ID NO:113 has 440 amino acid residues and is presented in Table 48B using the one-letter amino acid code. Signal P, Psort and/or Hydropathy results predict that NOV48 has no signal peptide and is likely to be localized in the plasma membrane with a certainty of 0.6000.

TABLE 48B
Encoded NOV48 protein sequence.

| | |
|---|---|
| MDGNDNVTLLFAPLLRDNYTLAPNASSLGPGTDLALAPASSAGPGPGLSLGPGPSFGFSP | (SEQ ID NO:114) |
| GPTPTPEPTTSGLAGGAASHGPSPFPRPWAPHALPFWDTPLNHGLNVFVGAALCITMLGL | |
| GCTVDVNHFGAHVRRPVGALLAALCQFGLLPLLAFLLALAFKLDEVAAVAVLLCGCCPGG | |
| NLSNLMSLLVDGDMNLSIIMTISSTLLALVLMPLCLWIYSWAWINTPIVQLLPLGTVTLT | |
| LCSTLIPIGLGVFIRYKYSRVADYIVKVRPVSLWSLLVTLVVLFIMTGTMLGPELLASIP | |
| AAVYVIAIFMPLAGYASGYGLATLFHLPPNCKRTVCLETGSQNVQLCTAILKLAFPPQFI | |
| GSMYMFPLLYALFQSAEAGIFVLIYKMYGSEMLHKRDPLDEDEDTDISYKKLKEEEMADT | |
| SYGTVKAENIIMMETAQTSL | |

A search of sequence databases reveals that the NOV48 amino acid sequence has 126 of 325 amino acid residues (38%) identical to, and 193 of 325 amino acid residues (59%) similar to, the 362 amino acid residue ptnr:SWISSPROT-ACC:O08705 protein from Mus musculus (Mouse) (SODIUM/BILE ACID COTRANSPORTER (NA(+)/BILE ACID COTRANSPORTER) (NA(+)/TAUROCHOLATE TRANSPORT PROTEIN) (SODIUM/TAUROCHOLATE COTRANSPORTING POLYPEPTIDE)). Public amino acid databases include the GenBank databases, SwissProt, PDB and PIR.

NOV48 is expressed in at least Liver. This information was derived by determining the tissue sources of the sequences that were included in the invention including but not limited to SeqCalling sources, Public EST sources, Literature sources, and/or RACE sources.

The disclosed NOV48 polypeptide has homology to the amino acid sequences shown in the BLASTP data listed in Table 48C.

TABLE 48C
BLAST results for NOV48

| Gene Index/ | | Length | Identity | Positives | |
|---|---|---|---|---|---|
| Identifier | Protein/Organism | (aa) | (%) | (%) | Expect |
| gi\|15082287\|gb\|AAH12048.1\| | protein for | 467 | 437/440 | 437/440 | 0.0 |
| AAH12048 | (IMAGE: 3502817) | | (99%) | (99%) | |
| (BC012048 | [Homo sapiens] | | | | |
| gi\|17512162\|gb\|AAH19066.1\| | protein for | 432 | 437/440 | 437/440 | 0.0 |
| AAH19066 | MGC: 29802) [Homo | | (99%) | (99%) | |
| (BC019066) | sapiens] | | | | |
| gi\|15294592\|ref\|XP_ | protein XP_053248 | 435 | 358/445 | 365/445 | e−66 |
| 053248.1\| | [Homo sapiens] | | (80%) | (81%) | |
| (XM_053248) | | | | | |
| gi\|3980315\|emb\|CAA10360.1\| | hepatic sodium- | 348 | 125/334 | 192/334 | 8e−54 |
| (AJ131361) | dependent bile | | (37%) | (57%) | |
| | acid transporter | | | | |
| | [Oryctolagus | | | | |
| | cuniculus] | | | | |
| gi\|2811069\|sp\|O08705\| | SODIUM/BILE ACID | 362 | 123/312 | 189/312 | e−52 |
| NTCP_MOUSE | COTRANSPORTER | | (39%) | (60%) | |
| | (NA (+)/BILE ACID | | | | |
| | COTRANSPORTER) | | | | |
| | (NA (+)/TAUROCHOLATE | | | | |
| | TRANSPORT | | | | |
| | PROTEIN) | | | | |
| | (SODIUM/TAUROCHOLATE | | | | |
| | COTRANSPORTING | | | | |
| | POLYPEPTIDE) | | | | |

Table 48D lists the domain descriptions from DOMAIN analysis results against NOV48. This indicates that the NOV48 sequence has properties similar to those of other proteins known to contain this domain.

TABLE 48D
Domain Analysis of NOV48

gnl|Pfam|pfam01758, SBF, Sodium Bile acid symporter family. This
family consists of Na+/bile acid co-transporters. These transmembrane
proteins function in the liver in the uptake of bile acids from portal
blood plasma a process mediated by the co-transport of Na+. Also in
the family is ARC3 from S. cerevisiae this is a putative transmembrane
protein involved in resistance to arsenic compounds.
CD-Length = 186 residues, 95.7% aligned
Score = 104 bits (259), Expect = 1e−23

AcDNA probe from a cloned rat liver Na+/taurocholate cotransporting polypeptide (Ntcp) was used to screen a human liver cDNA library. A 1,599-bp cDNA clone that encodes a human Na+/taurocholate cotransporting polypeptide (NTCP) was isolated. The human NTCP consists of 349 amino acids (calculated molecular mass of 38 kD) and exhibits 77% amino acid homology with the rat Ntcp. In vitro translation experiments indicate that the protein is glycosylated and has a molecular weight similar to the rat Ntcp. Injection of in vitro transcribed cRNA into Xenopus laevis oocytes resulted in the expression of Na(+)-dependent taurocholate uptake. Saturation kinetics indicated that the human NTCP has a higher affinity for taurocholate (apparent Km=6 microM) than the previously cloned rat protein (apparent Km=25 microM). NTCP-mediated taurocholate uptake into oocytes was inhibited by all major bile acid derivatives (100 microM), bumetanide (500 microM), and bromosulphophthalein (100 microM). Southern blot analysis of genomic DNA from a panel of human/hamster somatic cell hybrids mapped the human NTCP gene to chromosome 14. PMID: 8132774

Liver parenchymal cells continuously extract high amounts of bile acids from portal blood plasma. This uptake process is mediated by a Na+/bile acid cotransport system. A cDNA encoding the rat liver bile acid uptake system has been isolated by expression cloning in Xenopus laevis oocytes. The cloned transporter is strictly sodium-dependent and can be inhibited by various non-bile-acid organic compounds. Sequence analysis of the cDNA revealed an open reading frame of 1086 nucleotides coding for a protein of 362 amino acids (calculated molecular mass 39 kDa) with five possible N-linked glycosylation sites and seven putative transmembrane domains. Translation experiments in vitro and in oocytes indicate that the transporter is indeed glycosylated and that its polypeptide backbone has an apparent molecular mass of 33-35 kDa. Northern blot analysis with the cloned probe revealed crossreactivity with mRNA species from rat kidney and intestine as well as from liver tissues of mouse, guinea pig, rabbit, and man. PMID: 1961729

Using expression cloning in Xenopus laevis oocytes, a cDNA encoding a rat liver organic anion-transporting polypeptide (oatp) was isolated. The cloned oatp mediated Na(+)-independent uptake of sulfobromophthalein (BSP) which was Cl(−)-dependent in the presence of bovine serum albumin (BSA) at low BSP concentrations (e.g., 2 microM). Addition of increasing amounts of BSA had no effects on the maximal velocity of initial BSP uptake, but it increased the Km value from 1.5 microM (no BSA) to 24 microM (BSA/BSP molar ratio, 3.7) and 35 microM (BSA/BSP ratio, 18.4). In addition to BSP, the cloned oatp also mediated Na(+)-independent uptake of conjugated (taurocholate) and unconjugated (cholate) bile acids. Sequence analysis of the cDNA revealed an open reading frame of 2010 nucleotides coding for a protein of 670 amino acids (calculated molecular mass, 74 kDa) with four possible N-linked glycosylation sites and 10 putative transmembrane domains. Translation experiments in vitro indicated that the transporter was indeed glycosylated and that its polypeptide backbone had an apparent molecular mass of 59 kDa. Northern blot analysis with the cloned probe revealed crossreactivity with several mRNA species from rat liver, kidney, brain, lung, skeletal muscle, and proximal colon as well as from liver tissues of mouse and rabbit, but not of skate (Raja erinacea) and human. PMID: 8278353 Active uptake of bile acids from the lumen of the small intestine is mediated by an ileal Na(+)-dependent bile acid transport system. To identify components of this transport system, an expression cloning strategy was employed to isolate a hamster ileal cDNA that exhibits bile acid transport activity. By Northern blot analysis, mRNA for the cloned transporter was readily detected in ileum and kidney but was absent from liver and proximal small intestine. The transporter cDNA encoded a 348-amino acid protein with seven potential transmembrane domains and three possible N-linked glycosylation sites. The amino acid sequence was 35% identical and 63% similar to the rat liver Na+/bile acid cotransporter. After transfection into COS cells, the hamster cDNA transported taurocholate in a strict Na(+)-dependent fashion with an apparent Km of 33 microM. This taurocholate transport was inhibited by various bile acids but not by taurine or other organic anions. The Na+ dependence, saturability, and bile acid specificity of transport as well as the tissue specificity of mRNA expression strongly argue that the transporter cDNA characterized in this study is the Na+/bile acid cotransporter described previously in ileum. PMID: 8288599 Uptake of long-chain and aromatic neutral amino acids into cells is known to be catalyzed by the Na(+)-independent system L transporter, which is ubiquitous in animal cells and tissues. The 2.3-kilobase cDNA codes for a protein of 683 amino acids. The transporter has four putative membrane-spanning domains and bears no sequence or structural homology to any known animal or bacterial transporter. When transcribed and expressed in Xenopus oocytes, the transporter exhibits many, but not all, of the characteristics of L-system transporters, suggesting that this represents one of several related L-system transporters. PMID: 1729674

The phylogenic and ontogenic expression of mRNA for the Na+/bile acid cotransporter was determined by Northern analysis utilizing a full-length cDNA probe recently cloned from rat liver. mRNA was detected in several mammalian species, including rat, mouse, and man, but could not be found in livers from nonmammalian species, including chicken, turtle, frog, and small skate. When expression of the bile acid transporter in developing rat liver was studied, mRNA was detected between 18 and 21 days of gestation, at the time when Na(+)-dependent bile acid transport is first detected. Two hepatoma cell lines (HTC and HepG2), the latter of which is known to have lost the Na+/bile acid cotransport system, also did not express mRNA for this transporter. Finally, when mRNA from the lower vertebrate (the small skate) was injected into Xenopus oocytes, only a sodium-independent, chloride-dependent transport system for bile acids was expressed, confirming the integrity of the mRNA and consistent with prior functional studies of bile acid transport in this species. These findings establish that the Na+/bile acid cotransport mRNA is first transcribed in mammalian species, a process that is recapitulated late during mammalian fetal development in rat liver, and that this mRNA is lost in dedifferentiated hepatocytes. In contrast, the mRNA for a multispecific Na+/independent organic anion transport system is transcribed earlier in vertebrate evolution. PMID: 8421672

The disclosed NOV48 nucleic acid of the invention encoding a sodium/bile acid transporter-like protein includes the nucleic acid whose sequence is provided in Table 48A or a fragment thereof. The invention also includes a mutant or variant nucleic acid any of whose bases may be changed from the corresponding base shown in Table 48A while still encoding a protein that maintains its sodium/bile acid transporter-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37 percent of the bases may be so changed.

The disclosed NOV48 protein of the invention includes the sodium/bile acid transporter-like protein whose sequence is provided in Table 48B. The invention also includes a mutant or variant protein any of whose residues may be changed from the corresponding residue shown in Table 48B while still encoding a protein that maintains its sodium/bile acid transporter-like activities and physiological functions, or a functional fragment thereof. In the mutant or variant protein, up to about 62 percent of the residues may be so changed.

The invention further encompasses antibodies and antibody fragments, such as F_{ab} or (F_{ab})₂, that bind immunospecifically to any of the proteins of the invention.

The above defined information for this invention suggests that this sodium/bile acid transporter-like protein (NOV48) may function as a member of a “sodium/bile acid transporter family”. Therefore, the NOV48 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV48 nucleic acids and proteins of the invention are useful in potential therapeutic applications implicated in diseases including but not limited to various pathologies and disorders as indicated below. For example, a cDNA encoding the sodium/bile acid transporter-like protein (NOV48) may be useful in gene therapy, and the sodium/bile acid transporter-like protein (NOV48) may be useful when administered to a subject in need thereof. By way of nonlimiting example, the compositions of the present invention will have efficacy for treatment of patients suffering from cancer, trauma, regeneration (in vitro and in vivo), viral/bacterial/parasitic infections, Von Hippel-Lindau (VHL) syndrome, Cirrhosis, Transplantation, or other pathologies or conditions. The NOV48 nucleic acid encoding the sodium/bile acid transporter-like protein of the invention, or fragments thereof, may further be useful in diagnostic applications, wherein the presence or amount of the nucleic acid or the protein are to be assessed.

NOV48 nucleic acids and polypeptides are further useful in the generation of antibodies that bind immuno-specifically to the novel NOV48 substances for use in therapeutic or diagnostic methods. These antibodies may be generated according to methods known in the art, using prediction from hydrophobicity charts, as described in the “Anti-NOVX Antibodies” section below. The disclosed NOV48 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV49

A disclosed NOV49 nucleic acid of 2313 nucleotides (also referred to as CG57721-01) encoding a prestin-like protein is shown in Table 49A. The start and stop codons are in bold letters.

TABLE 49A
NOV49 nucleotide sequence.

| | |
|---|---|
| CCATTGACTGCAGGAAGGTTGGCCAGCAGAGCAAATGCCATGCCTGCAGAGGACAACGAG | (SEQ ID NO:115) |
| ACTGAAGCTCAGCAGCATCACCGCTACGTGGTAGACAGAGCCGCATACTCCCTTACCCTC | |
| TTCGACGATGAGTTTGAGAAGAAGGACCGGACATACCCAGTGGGAGAGAAACTTCGCAAT | |
| GCCTTCAGATGTTCCTCAGCCAAGATCAAAGCTGTGGTGTTTGGGCTGCTGCCTGTGCTC | |
| TCCTGGCTCCCCAAGTACAAGATTAAAGACTACATCATTCCTGACCTGCTCGGTGGACTC | |
| AGCGGGGGATCCATCCAGGTCCCACAAGGTATGGCATTTGCTCTGCTGGCCAACCTTCCT | |
| GCAGTCAATGGCCTCTACTCCTCCTTCTTCCCCCTCCTGACCTACTTCTTCCTGGGGGGT | |
| GTTCACCAGATGGTGCCAGGTACCTTTGCCGTTATCAGCATCCTGGTGGGTAACATCTGT | |
| CTGCAGCTGGCCCCAGAGTCGAAATTCCAGGTCTTCAACAATGCCACCAATGAGAGCTAT | |
| GTGGACACAGCAGCCATGGAGGCTGAGAGGCTGCACGTGTCAGCTACGCTAGCCTGCCTC | |
| ACCGCCATCATCCAGATGGGTCTGGGCTTCATGCAGTTTGGCTTTGTGGCCATCTACCTC | |
| TCCGAGTCCTTCATCCGGGGCTTCATGACGGCCGCCGGCCTGCAGATCCTGATTTCGGTC | |
| CTCAAGTACATCTTCGGACTGACCATCCCCTCCTACACAGGCCCAGGGTCCATCGTCTTT | |
| GTGAGTCTGGGGATGTGCAAAAACCTCCCCCACACCAACATCGCCTCGCTCATCTTCGCT | |
| CTCATCAGCGGTGCCTTCCTGGTGCTGGTGAAGGAGCTCAATGCTCGCTACATGCACAAG | |
| ATTCGCTTCCCCATCCCTACAGAGATGATTGTGGTAAGGACCTTGTTCAGAGCTGGGTGT | |
| AAGATGCCCAAAAAGTATCACATGCAGATCGTGGGAGAAATCCAACTCGGCAGGTTCCCC | |
| ACCCCGGTGTCGCCTGTGGTCTCACAGTGGAAGGACATGATAGGCACAGCCTTCTCCCTA | |
| GCCATCGTGAGCTACGTCATCAACCTGGCTATGGGCCGGACCCTGGCCAACAAGCACGGC | |
| TACGACGTGGATTCGAACCAGGAGATGATCGCTCTCGGCTGCAGCAACTTCTTTGGCTCC | |
| TTCTTTAAAATTCATGTCATTTGCTGTGCGCTTTCTGTCACTCTGGCTGTGGATGGAGCT | |
| GGAGGAAAATCCCAGGTGAGCCTTGTTCTAGGGGAGTTGTCTGAGCTCCCCTTCTTACTC | |
| ACCACGGGGTTTGCTTTAAGAGTACTCAGGTGTCTCTCTGTGCTAGGAGCCCTGATCGCT | |
| GTCAATCTCAAGAACTCCCTCAAGCAACTCACCGACCCCTACTACCTGTGGAGGAAGAGC | |
| AAGCTGGACCAGTGCATCTGGGTAGTGAGCTTCCTCTCCTCCTTCTTCCTCAGCCTGCCC | |
| TATGGTGTGGCAGTGGGTGTCGCCTTCTCCGTCCTGGTCGTGGTCTTCCAGACTCAGAGT | |
| CGAAATGGCTATGCACTGGCCCAGGTCATGGACACTGACATTTATGTGAATCCCAAGACC | |
| TATAATAGGGTACAGGATATCCAGGGGATTAAAATCATCACGTACTGCTCCCCTCTCTAC | |
| TTTGCCAACTCAGAGATCTTCAGGCAAAAGGTCATCGCCAAGGTAAGGCTCAGTCCCTGG | |
| CGACCAGAGGCTCTGGACAGAGAGTGGCCGGAAAATGGAAGCAGAAGGGCGGTGGGACCC | |
| AACAACAACCAGACCCCGGCTAACGGCACCAGCGTGTCCTATATCACCTTCAGCCCTGAC | |
| AGCTCCTCACCTGCCCAGAGTGAGCCACCAGCCTCCGCTGAGGCCCCCGGCGAGCCCAGT | |
| GACATGCTGGCCAGCGTCCCACCCTTCGTCACCTTCCACACCCTCATCCTCGACATGAGT | |
| GGAGTCAGCTTCGTGGACTTGATGGGCATCAAGGCCCTGGCCAAGCTGAGCTCCACCTAT | |
| GGGAAGATCGGCGTAAAGGTCTTCTTGGTGAACATCCATGCCCAGGTGTACAATGACATT | |
| AGCCATGGAGGCGTCTTTGAGGATGGGAGCCTAGGATGCAAGCACGTCTTTCCCAGCATA | |
| CATGACGCAGTCCTCTTTGCCCAGCTGATTCAGTTACCTGGATTGAGGTCACTGGCAATG | |
| GCTGAAGTGGAGACGCAGGTGGAACTGGTTCAGGCCGGGGGAATCACCCACTTGAGTTTG | |
| TACTAAAAGCCCCAGCCCAGCCCTGTTTCTCTT | |

In a search of public sequence databases, the NOV49 nucleic acid sequence, located on chromsome 3 has 966 of 1618 bases (59%) identical to a gb:GENBANK-ID:AF279265|acc:AF279265.1 mRNA from Homo sapiens (Homo sapiens putative anion transporter 1 mRNA, complete cds). Public nucleotide databases include all GenBank databases and the GeneSeq patent database.

The disclosed NOV49 polypeptide (SEQ ID NO:116) encoded by SEQ ID NO:115 has 748 amino acid residues and is presented in Table 49B using the one-letter amino acid code. Signal P, Psort and/or Hydropathy results predict that NOV49 has a signal peptide and is likely to be localized at the plasma membrane with a certainty of 0.6000. The most likely cleavage site for a NOV49 peptide is between amino acids 8 and 9.

TABLE 49B
Encoded NOV49 protein sequence.

| | |
|---|---|
| MPAEDNETEAQQHDRYVVDRAAYSLTLFDDEFEKKDRTYPVGEKLRNAFRCSSAKIKAVV | (SEQ ID NO:116) |
| FGLLPVLSWLPKYKIKDYIIPDLLGGLSGGSIQVPQGMAFALLANLPAVNGLYSSFFPLL | |
| TYFFLGGVHQMVPGTFAVISILVGNICLQLAPESKFQVFNNATNESYVDTAAMEAERLHV | |
| SATLACLTAIIQMGLGFMQFGFVAIYLSESFIRGFMTAAGLQILISVLKYIFGLTIPSYT | |
| GPGSIVFVSLGMCKNLPHTNIASLIFALISGAFLVLVKELNARYMHKIRFPIPTEMIVVR | |
| TLFRAGCKMPKKYHMQIVGEIQLGRFPTPVSPVVSQWKDMIGTAFSLAIVSYVINLAMGR | |
| TLANKHGYDVDSNQEMIALGCSNFFGSFFKIHVICCALSVTLAVDGAGGKSQVSLVLGEL | |
| SELPFLLTTGFALRVLRCLSVLGALIAVNLKNSLKQLTDPYYLWRKSKLDQCIWVVSFLS | |
| SFFLSLPYGVAVGVAFSVLVVVFQTQSRNGYALAQVMDTDIYVNPKTYNRVQDIQGIKII | |
| TYCSPLYFANSEIFRQKVIAKVRLSPWRPEALDREWPENGSRRAVGPNNNQTPANGTSVS | |
| YITFSPDSSSPAQSEPPASAEAPGEPSDMLASVPPFVTFHTLILDMSGVSFVDLMGIKAL | |
| AKLSSTYGKIGVKVFLVNIHAQVYNDISHGGVFEDGSLGCKHVFPSIHDAVLFAQLIQLP | |
| GLRSLAMAEVETQVELVQAGGITHLSLY | |

A search of sequence databases reveals that the NOV49 amino acid sequence has 277 of 732 amino acid residues (37%) identical to, and 434 of 732 amino acid residues (59%) similar to, the 744 amino acid residue ptnr:TREMBLNEW-ACC:CAC21555 protein from Rattus norvegicus (Rat) (PRESTIN). Public amino acid databases include the GenBank databases, SwissProt, PDB and PIR.

NOV49 is expressed in at least Parotid Salivary glands. Expression information was derived from the tissue sources of the sequences that were included in the derivation of the sequence of CG57721-0. The sequence is predicted to be expressed in the following tissues because of the expression pattern of (GENBANK-ID: gb:GENBANK-ID:A1279265|acc:AF279265.1) a closely related 1—Homo sapiens putative anion transporter 1 mRNA, complete cds homolog in species Homo sapiens: kidney and pancreas. This information was derived by determining the tissue sources of the sequences that were included in the invention including but not limited to SeqCalling sources, Public EST sources, Literature sources, and/or RACE sources.

The disclosed NOV49 polypeptide has homology to the amino acid sequences shown in the BLASTP data listed in Table 49C.

TABLE 49C
BLAST results for NOV49

| Gene Index/ | | Length | Identity | Positives | |
|---|---|---|---|---|---|
| Identifier | Protein/Organism | (aa) | (%) | (%) | Expect |
| gi\|16588681\|gb\|AAL26867.1\| | anion | 887 | 642/739 | 657/739 | 0.0 |
| AF314958_1 | transporter/exchanger-9 | | (86%) | (88%) | |
| (AF314958) | [Homo | | | | |
| | sapiens ] | | | | |
| gi\|16418413\|ref\|NP_ | solute carrier | 791 | 642/739 | 657/739 | 0.0 |
| 443166.1\| | family 26, member | | (86%) | (88%) | |
| (NM_052934) | 9 [Homo sapiens] | | | | |
| gi\|15011891\|ref\|NP_ | prestin (motor | 744 | 281/748 | 430/748 | e−129 |
| 109652.2\| | protein) [Mus | | (37%) | (56%) | |
| (NM_030727) | musculus] | | | | |
| gi\|13540646\|ref\|NP_ | prestin [Rattus | 744 | 278/746 | 435/746 | e−128 |
| 110467.1\| | norvegicus] | | (37%) | (58%) | |
| (NM_030840) | | | | | |
| gi\|8050590\|gb\|AAF71715.1\| | prestin [Meriones | 744 | 271/738 | 424/738 | e−125 |
| AF230376_1 | unguiculatus] | | (36%) | (56%) | |
| (AF230376) | | | | | |

Tables 49D-E list the domain descriptions from DOMAIN analysis results against NOV49. This indicates that the NOV49 sequence has properties similar to those of other proteins known to contain this domain.

TABLE 49D
Domain Analysis of NOV49

gnl|Pfam|pfam00916, Sulfate_transp, Sulfate transporter family.
Mutations in human Diastrophic Dysplasia Protein lead to several diseases.
CD-Length = 312 residues, 100.0% aligned
Score = 167 bits (423), Expect = 2e−42

TABLE 49E
Domain Analysis of NOV49

gnl|Pfam|pfam01740, STAS, STAS domain. The STAS (after Sulphate
Transporter and AntiSigma factor antagonist) domain is found in the C
terminal region of Sulphate transporters and bacterial antisigma
factor antagonists. It has been suggested that this domain may have a
general NTP binding function.
CD-Length = 106 residues, 62.3% aligned
Score = 57.0 bits (136), Expect = 4e−09

A second distinct family of anion transporters, in addition to the classical SLC4 (or AE) family, has recently been delineated. Members of the SLC26 family are structurally well conserved and can mediate the electroneutral exchange of Cl(−) for HCO(−)(3) across the plasma membrane of mammalian cells like members of the SLC4 family. Three human transporter proteins have been functionally characterized: SLC26A2 (DTDST), SLC26A3 (CLD or DRA), and SLC26A4 (PDS) can transport with different specificities the chloride, iodine, bicarbonate, oxalate, and hydroxyl anions, whereas SLC26A5 (prestin) was suggested to act as the motor protein of the cochlear outer hair cell.

Electromotility, i.e., the ability of cochlear outer hair cells (OHCs) to contract and elongate at acoustic frequencies, is presumed to depend on the voltage-driven conformational changes of “motor” proteins present in the OHC lateral plasma membrane. Recently, two membrane proteins have been proposed as candidates for the OHC motor. A sugar transporter, GLUT-5, was proposed based on its localization in the OHCs and on the observation that sugar transport alters the voltage sensitivity of the OHC motor mechanism. Another candidate, “prestin,” was identified from a subtracted OHC cDNA library and shown to impart voltage-driven shape changes to transfected cultured cells.

The disclosed NOV49 nucleic acid of the invention encoding a prestin-like protein includes the nucleic acid whose sequence is provided in Table 49A or a fragment thereof. The invention also includes a mutant or variant nucleic acid any of whose bases may be changed from the corresponding base shown in Table 49A while still encoding a protein that maintains its prestin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In the mutant or variant nucleic acids, and their complements, up to about 41 percent of the bases may be so changed.

The disclosed NOV49 protein of the invention includes the prestin-like protein whose sequence is provided in Table 49B. The invention also includes a mutant or variant protein any of whose residues may be changed from the corresponding residue shown in Table 49B while still encoding a protein that maintains its prestin-like activities and physiological functions, or a functional fragment thereof. In the mutant or variant protein, up to about 63 percent of the residues may be so changed.

The invention further encompasses antibodies and antibody fragments, such as F_{ab} or (F_{ab})₂, that bind immunospecifically to any of the proteins of the invention.

The above defined information for this invention suggests that this prestin-like protein (NOV49) may function as a member of a “prestin family”. Therefore, the NOV49 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV49 nucleic acids and proteins of the invention are useful in potential therapeutic applications implicated in diseases including but not limited to various pathologies and disorders as indicated below. For example, a cDNA encoding the prestin-like protein (NOV49) may be useful in gene therapy, and the prestin-like protein (NOV49) may be useful when administered to a subject in need thereof. By way of nonlimiting example, the compositions of the present invention will have efficacy for treatment of patients suffering from Autoimmune disease, Renal artery stenosis, Interstitial nephritis, Glomerulonephiritis, Polycystic kidney disease, Systemic lupus erythematosus, Renal tubular acidosis, IgA nephropathy, Hypercalceimia, Lesch-Nyhan syndrome, Diabetes, Von Hippel-Lindau (VHL) syndrome, Pancreatitis, Obesity, Xerostomia, cancer, trauma, regeneration (in vitro and in vivo), viral/bacterial/parasitic infections, or other pathologies or conditions. The NOV49 nucleic acid encoding the prestin-like protein of the invention, or fragments thereof, may further be useful in diagnostic applications, wherein the presence or amount of the nucleic acid or the protein are to be assessed.

NOV49 nucleic acids and polypeptides are further useful in the generation of antibodies that bind immuno-specifically to the novel NOV49 substances for use in therapeutic or diagnostic methods. These antibodies may be generated according to methods known in the art, using prediction from hydrophobicity charts, as described in the “Anti-NOVX Antibodies” section below. The disclosed NOV49 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV50

A disclosed NOV50 nucleic acid of 1335 nucleotides (also referred to as CG57787-01) encoding a sulfate transporter-like protein is shown in Table 50A. The start and stop codons are in bold letters.

TABLE 50A
NOV50 nucleotide sequence.

(SEQ ID NO:117)

GCCTTCCTGTCCGGCTGCATCCAGCTGGCCATGGGGGTCCTGCGTTTGGGGTTCCTGCTGGACTTCATTT
CCTACCCCGTCATTAAAGGCTTCACCTCTGCTGCTGCCGTCACCATCGGCTTTGGACAGATCAAGAACCT
GCTGGGACTACAGAACATCCCCAGGCCGTTCTTCCTGCAGGTGTACCACACCTTCCTCAGGATTGCAGAG
ACCAGGGTAGGTGACGCCGTCCTGGGGCTGGTCTGCATGCTGCTGCTGCTGGTGCTGAAGCTGATGCGGG
ACCACGTGCCTCCCGTCCACCCCGAGATGCCCCCTGGTGTGCGGCTCAGCCGTGGGCTGGTCTGGGCTGC
CACGACAGCTCGCAACGCCCTGGTGGTCTCCTTCGCAGCCCTGGTTGCGTACTCCTTCGAGGTGACTGGA
TACCAGCCTTTCATCCTAACAGGGGAGACAGCTGAGGGGCTCCCTCCAGTCCGGATCCCGCCCTTCTCAG
TGACCACAGCCAACGGGACGATCTCCTTCACCGAGATGGTGCAGGACATGGGAGCCGGGCTGGCCGTGGT
GCCCCTGATGGGCCTCCTGGAGAGCATTGCGGTGGCCAAAGCCTTCGCATCTCAGAATAATTACCGCATC
GATGCCAACCAGGAGCTGCTGGCCATCGGTCTCACCAACATGTTGGGCTCCCTCGTCTCCTCCTACCCGG
TCACAGGCAGCTTTGGACGGACAGCCGTGAACGCTCAGTCGGGGGTGTGCACCCCGGCGGGGGGCCTGGT
GACGGGAGTGCTGGTGCTGCTGTCTCTGGACTACCTGACCTCACTGTTCTACTACATCCCCAAGTCTGCC
CTGGCTGCCGTCATCATCATGGCCGTGGCCCCGCTGTTCGACACCAAGATCTTCAGGACGCTCTGGCGTG
TTAAGAGGCTGGACCTGCTGCCCCTGTGCGTGACCTTCCTGCTGTGCTTCTGGGAGGTGCAGTACGGCAT
CCTGGCCGGGGCCCTGGTGTCTCTGCTCATGCTCCTGCACTCTGCAGCCAGGCCTGAGACCAAGGTGTCA
GAGGGGCCGGTTCTGGTCCTGCAGCCGGCCAGCGGCCTGTCCTTCCCTGTCCTCTGCCCCCCACTCCCTG
CTGTTCAGGACCCCAAGACCCTGTCCCCGACGCTCTCCAGTCCACAAGGATGCAGGCATCTCTGAGTGGG
CTGGACCGTCCTCTGTGGCCCTCAGCCAGTGGTGCTGCAGCAAGGGTGGTGGCTCCCCACATATCACTCC
TTCCCTGCCCCTAAAGTCCGGTTCCTGTTTCTGGGGGGTTGATTTTAGGGGAGCTAAGGGCCTGTGAGTC
CTAGT

In a search of public sequence databases, the NOV50 nucleic acid sequence has 585 of 993 bases (58%) identical to a gb:GENBANK-ID:AF297659|acc:AF297659.2 mRNA from Homo sapiens (Homo sapiens sulfate/anion transporter SAT-1 protein (SLC26AI) mRNA, complete cds). Public nucleotide databases include all GenBank databases and the GeneSeq patent database.

The disclosed NOV50 polypeptide (SEQ ID NO:118) encoded by SEQ ID NO:117 has 384 amino acid residues and is presented in Table 50B using the one-letter amino acid code. Signal P, Psort and/or Hydropathy results predict that NOV50 has a signal peptide and is likely to be localized extracellularly with a certainty of 0.6000. The most likely cleavage site for a NOV50 peptide is between amino acids 20 and 21.

TABLE 50B
Encoded NOV50 protein sequence.

| | |
|---|---|
| MGVLRLGFLLDFISYPVIKGFTSAAAVTIGFGQIKNLLGLQNIPRPFFLQVYHTFLRIAE | (SEQ ID NO:118) |
| TRVGDAVLGLVCMLLLLVLKLMRDHVPPVHPEMPPGVRLSRGLVWAATTARNALVVSFAA | |
| LVAYSFEVTGYQPFILTGETAEGLPPVRIPPFSVTTANGTISFTEMVQDMGAGLAVVPLM | |
| GLLESIAVAKAFASQNNYRIDANQELLAIGLTNMLGSLVSSYPVTGSFGRTAVNAQSGVC | |
| TPAGGLVTGVLVLLSLDYLTSLFYYIPKSALAAVIIMAVAPLFDTKIFRTLWRVKRLDLL | |
| PLCVTFLLCFWEVQYGILAGALVSLLMLLHSAARPETKVSEGPVLVLQPASGLSFPVLCP | |
| PLPAVQDPKTLSPTLSSPQGCRHL | |

A search of sequence databases reveals that the NOV50 amino acid sequence has 146 of 339 amino acid residues (43%) identical to, and 210 of 339 amino acid residues (61%) similar to, the 595 amino acid residue ptnr:SPTREMBL-ACC:Q9V879 protein from Drosophila melanogaster (Fruit fly) (CG5002 PROTEIN). Public amino acid databases include the GenBank databases, SwissProt, PDB and PIR.

NOV50 is expressed in at least Brain, Cerebral Medulla/Cerebral white matter, Coronary Artery, Epidermis, Frontal Lobe, Hippocampus, Hypothalamus, Kidney, Liver, Lung, Mammary gland/Breast, Oviduct/Uterine Tube/Fallopian tube, Pituitary Gland, Retina, Spinal Chord, Spleen, Substantia Nigra, Temporal Lobe, Testis, Umbilical Vein, Whole Organism. Expression information was derived from the tissue sources of the sequences that were included in the derivation of the sequence of CG57787_{—}01. The sequence is predicted to be expressed in the following tissues because of the expression pattern of (GENBANK-ID: gb:GENBANK-ID:AF297659|acc:AF297659.2) a closely related Homo sapiens sulfate/anion transporter SAT-1 protein (SLC26AI) mRNA, complete cds homolog in species Homo sapiens liver. This information was derived by determining the tissue sources of the sequences that were included in the invention including but not limited to SeqCalling sources, Public EST sources, Literature sources, and/or RACE sources.

The disclosed NOV50 polypeptide has homology to the amino acid sequences shown in the BLASTP data listed in Table 50C.

TABLE 50C
BLAST results for NOV50

| Gene Index/ | | Length | Identity | Positives | |
|---|---|---|---|---|---|
| Identifier | Protein/Organism | (aa) | (%) | (%) | Expect |
| gi\|18485058\|ref\|XP_ | CG5002 | 595 | 121/341 | 169/341 | 1e−46 |
| 080006.1\| | [Drosophila | | (35%) | (49%) | |
| (XM_080006) | melanogaster] | | | | |
| gi\|7301216\|gb\|AAF56347.1\| | Esp gene product | 654 | 124/368 | 180/368 | 7e−44 |
| (AE003749) | [Drosophila | | (33%) | (48%) | |
| | melanogaster] | | | | |
| gi\|17738183\|ref\|NP_ | Epidermal stripes | 623 | 127/370 | 179/370 | 3e−43 |
| 524490.1\| | and patches | | (34%) | (48%) | |
| (NM_079766 | [Drosophila | | | | |
| | melanogaster] | | | | |
| gi\|7301881\|gb\|AAF56989.1\| | CG7912 gene | 573 | 120/323 | 170/323 | 4e−42 |
| (AE003772 | product | | (37%) | (52%) | |
| | [Drosophila | | | | |
| | melanogaster | | | | |
| gi\|7300023\|gb\|AAF55195.1\| | CG6125 gene | 640 | 100/315 | 162/315 | 3e−38 |
| (AE003708) | product | | (31%) | (50%) | |
| | [Drosophila | | | | |
| | melanogaster] | | | | |

Table 50D lists the domain descriptions from DOMAIN analysis results against NOV50. This indicates that the NOV50 sequence has properties similar to those of other proteins known to contain this domain.

TABLE 50D
Domain Analysis of NOV50

gnl|Pfam|pfam00916, Sulfate_transp, Sulfate transporter family.
Mutations in human Diastrophic Dysplasia Protein lead to several diseases.
CD-Length = 312 residues, 95.2% aligned
Score = 163 bits (413), Expect = 1e−41

Lymphocytes continuously recirculate from blood, through lymphoid and other tissues, and back through the efferent lymphatics to the blood. The first critical step in lymphocyte migration from circulation into lymphoid tissues is the adhesion of lymphocytes to specialized postcapillary vascular sites called high endothelial venules (11EV).

Two vertebrate sulfate transporters that play a role in sulfate incorporation in other tissues are members of the superfamily of anion exchangers: the diastrophic dysplasia sulfate transporter (DTDST; 222600), which is mutant in diastrophic dysplasia and certain other skeletal dysplasias, and downregulated in adenoma (DRA; 126650), which is mutant in familial chloride diarrhea (214700). These 2 sulfate transporters contain 12 membrane-spanning domains and are sensitive to the anion-exchanger inhibitor DIDS. Girard et al. (1999) showed that HEVECs express 2 functional classes of sulfate transporters defined by their differential sensitivity to the DIDS anion-exchanger inhibitor. They reported the molecular characterization of a DIDS-resistant sulfate transporter from human HEVECs, designated SUT1. SUT1 belongs to the family of sodium-coupled anion transporters and exhibits 40 to 50% amino acid identity with the rat renal sodium/sulfate cotransporter NaSi1, as well as with the human and rat sodium/dicarboxylate cotransporters NADC1/SDCT1 (604148) and NADC3/SDCT2.

The disclosed NOV50 nucleic acid of the invention encoding a sulfate transporter-like protein includes the nucleic acid whose sequence is provided in Table 50A or a fragment thereof. The invention also includes a mutant or variant nucleic acid any of whose bases may be changed from the corresponding base shown in Table 50A while still encoding a protein that maintains its sulfate transporter-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 42 percent of the bases may be so changed.

The disclosed NOV50 protein of the invention includes the sulfate transporter-like protein whose sequence is provided in Table 50B. The invention also includes a mutant or variant protein any of whose residues may be changed from the corresponding residue shown in Table 50B while still encoding a protein that maintains its sulfate transporter-like activities and physiological functions, or a functional fragment thereof. In the mutant or variant protein, up to about 57 percent of the residues may be so changed.

The invention further encompasses antibodies and antibody fragments, such as F_{ab} or (F_{ab})₂, that bind immunospecifically to any of the proteins of the invention.

The above defined information for this invention suggests that this sulfate transporter-like protein (NOV50) may function as a member of a “sulfate transporter family”. Therefore, the NOV50 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV50 nucleic acids and proteins of the invention are useful in potential therapeutic applications implicated in diseases including but not limited to various pathologies and disorders as indicated below. For example, a cDNA encoding the sulfate transporter-like protein (NOV50) may be useful in gene therapy, and the sulfate transporter-like protein (NOV50) may be useful when administered to a subject in need thereof. By way of nonlimiting example, the compositions of the present invention will have efficacy for treatment of patients suffering from diastrophic dysplasia and certain other skeletal dysplasias, and adenoma, familial chloride diarrhea, CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV50 nucleic acid encoding the sulfate transporter-like protein of the invention, or fragments thereof, may further be useful in diagnostic applications, wherein the presence or amount of the nucleic acid or the protein are to be assessed.

NOV50 nucleic acids and polypeptides are further useful in the generation of antibodies that bind immuno-specifically to the novel NOV50 substances for use in therapeutic or diagnostic methods. These antibodies may be generated according to methods known in the art, using prediction from hydrophobicity charts, as described in the “Anti-NOVX Antibodies” section below. The disclosed NOV50 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV51

A disclosed NOV51 nucleic acid of 2079 nucleotides (also referred to as CG57785-01) encoding a sulfate transporter-like protein is shown in Table 51A. The start and stop codons are in bold letters.

TABLE 51A
NOV51 nucleotide sequence.

(SEQ ID NO:119)

ATGGTTGTGGCTGTCACAGATTTTAACTCCTCGGTGCACCAAGAGTACAGATTCCAAATTGCCTATATCT
TAAAAACTTGTAAGAAAGAAGCAATGGTGAATGTGAATCTGAACACCAGGGAGAGTTCCAGAAAGGGAAT
TCCCATCAGTTGGTACTACCTAATAATGGGCGTATTGGGTTTGGGCTTCATTGCCACTTACCTTCCGGAC
TCTGCAATGAGTGCTTACCTGGCTGCTGTGGCACTTCATATCATGCTGTCCCAGCTGACTTTCATCTTTG
GGATTATGATTAGTTTCCATGCCGGTCCCATCTCCTTCTTCTATGACATAATTAATTACTGTGTAGCTCT
CCCAAAAGCGAATTCCACCAGCATTCTAGTATTTCTAACTGTTGTTGTTGCTCTGCGAATCAACAAATGT
ATCAGAATTTCTTTCAATCAGTATCCCATTGAGTTTCCCATGGAATTATTTCTGATTATTGGCTTCACTG
TGATTGCAAACAAGATAAGCATGGCCACAGAAACCAGCCAGACGCTTATTGACATGATTCCTTATAGCTT
TCTGCTTCCTGTAACACCAGATTTCAGCCTTCTTCCCAAGATAATTTTACAAGCCTTCTCCTTATCTTTG
GTGAGCTCCTTTCTGCTCATATTTCTGGGCAAGAAGATTGCCAGTCTTCACAATTACAGTGTCAATTCCA
ACCAGGATTTAATAGCCATCGGCCTTTGCAATGTCGTCAGTTCATTTTTCAGATCTTGTGTGTTTACTGG
TGCTATTGCTAGGACTATTATCCAGGATAAATCTGGAGGAAGACAACAGTTTGCATCTCTGGTAGGCGCA
GGTGTGATGCTGCTCCTGATGGTGAAGATGGGACACTTTTTCTACACACTGCCAAATGTTGATATGGTAA
AGGTGCCTCTTAAAGAAGAAGAAATTTTCAGCTTGTTTAATTCAAGTGACACCAATCTACAAGGAGGAAA
GATTTGCAGGTGTTTCTGCAACTGTGATGATCTGGAGCCGCTGCCCAGGATTCTTTACACAGAGCGATTT
GAAAATAAACTGGATCCCGAAGCATCCTCCATTAACCTGATTCACTGCTCACATTTTGAGAGCATGAACA
CAAGCCAAACTGCATCCGAAGACCAAGTGCCATACACAGTATCGTCCGTGTCTCAGAAAAATCAAGGGCA
ACAGTATGAGGAGGTGGAGGAAGTTTGGCTTCCTAATAACTCATCAAGAAACAGCTCACCAGGACTGCCT
GATGTGGCGGAAAGCCAGGGGAGGAGATCACTCATCCCTTACTCAGATGCGTCTCTACTGCCCAGTGTCC
ACACCATCATCCTGGATTTCTCCATGGTACACTACGTGGATTCACGGGGGTTAGTCGTATTAAGACAGAT
ATGCAATGCCTTTCAAAACGCCAACATTTTGATACTCATTGCAGGGTGTCACTCTTCCATAGTCAGGGCA
TTTGAGAGGAATGATTTCTTTGACGCTGGCATCACCAAGACCCAGCTGTTCCTCAGCGTTCACGACGCCG
TGCTGTTTGCCTTGTCAAGGAAGGTCATAGGCTCCTCTGAGTTAAGCATCGATGAATCCGAGACAGTGAT
ACGGGAAACCTACTCAGAAACAGACAAGAATGACAATTCAAGATATAAAATGAGCAGCAGTTTTCTAGGA
AGCCAAAAAAATGTAAGTCCAGGCTTCATCAAGATCCAACAGCCTGTAGAAGAGGAGTCGGAGTTGGATT
TGGAGCTGGAATCAGAACAAGAGGCTGGGCTGGGTCTGGACCTAGACCTGGATCGGGAGCTGGAGCCTGA
AATGGAGCCCAAGGCTGAGACCGAGACCAAGACCCAGACCGAGATGGAGCCCCAGCCTGAGACTGAGCCT
GAGATGGAGCCCAACCCCAAATCTAGGCCAAGAGCTCACACTTTTCCTCAGCAGCGTTACTGGCCTATGT
ATCATCCGTCTATGGCTTCCACCCAGTCTCAGACTCAGACTCGGACATGGTCAGTGGAGAGGAGACGCCA
TCCTATGGATTCATACTCACCAGAGGGCAACAGCAATGAAGATGTCTAG

In a search of public sequence databases, the NOV51 nucleic acid sequence, located on chromsome 6 has 128 of 198 bases (64%) identical to a gb:GENBANK-ID:AF189262|acc:AF189262.1 mRNA from Rattus norvegicus (Rattus norvegicus GABA-A receptor epsilon-like subunit (Epsilon) mRNA, complete cds). Public nucleotide databases include all GenBank databases and the GeneSeq patent database.

The disclosed NOV51 polypeptide (SEQ ID NO:120) encoded by SEQ ID NO:119 has 692 amino acid residues and is presented in Table 51B using the one-letter amino acid code. Signal P, Psort and/or Hydropathy results predict that NOV51 has a signal peptide and is likely to be localized in the plasma membrane with a certainty of 0.6000. The most likely cleavage site for a NOV51 peptide is between amino acids 69 and 70.

TABLE 51B
Encoded NOV51 protein sequence.

| | |
|---|---|
| MVVAVTDFNSSVHQEYRFQIAYILKTCKKEAMVNVNLNTRESSRKGIPISWYYLIMGVLG | (SEQ ID NO:120) |
| LGFIATYLPESAMSAYLAAVALHIMLSQLTFIFGIMISFHAGPISFFYDIINYCVALPKA | |
| NSTSILVFLTVVVALRINKCIRISFNQYPIEFPMELFLIIGFTVIANKISMATETSQTLI | |
| DMIPYSFLLPVTPDFSLLPKIILQAFSLSLVSSFLLIFLGKKIASLHNYSVNSNQDLIAI | |
| GLCNVVSSFFRSCVFTGAIARTIIQDKSGGRQQFASLVGAGVMLLLMVKMGHFFYTLPNV | |
| DMVKVPLKEEEIFSLFNSSDTNLQGGKICRCFCNCDDLEPLPRILYTERFENKLDPEASS | |
| INLIHCSHFESMNTSQTASEDQVPTTVSSVSQKNQGQQYEEVEEVWLPNNSSRNSSPGLP | |
| DVAESQGRRSLIPYSDASLLPSVHTIILDFSMVHYVDSRGLVVLRQICNAFQNANILILI | |
| AGCHSSIVRAFERNDFFDAGITKTQLFLSVHDAVLFALSRKVIGSSELSIDESETVIRET | |
| YSETDKNDNSRYKMSSSFLGSQKNVSPGFIKIQQPVEEESELDLELESEQEAGLGLDLDL | |
| DRELEPEMEPKAETETKTQTEMEPQPETEPEMEPNPKSRPRAHTFPQQRYWPMYHPSMAS | |
| TQSQTQTRTWSVERRRHPMDSYSPEGNSNEDV | |

A search of sequence databases reveals that the NOV51 amino acid sequence has 123 of 123 amino acid residues (100%) identical to, and 123 of 123 amino acid residues (100%) similar to, the 123 amino acid residue ptnr:SPTREMBL-ACC:Q9NQP0 protein from Homo sapiens (Human) (BA48209.2 (NOVEL SULPHATE TRANSPORTER FAMILY MEMBER)). Public amino acid databases include the GenBank databases, SwissProt, PDB and PIR.

NOV51 is expressed in at least Adipose, Peripheral Blood, Spinal Chord, Testis, and Colon. Expression information was derived from the tissue sources of the sequences that were included in the derivation of the sequence of CG57785_{—}01. The sequence is predicted to be expressed in the following tissues because of the expression pattern of (GENBANK-ID: gb:GENBANK-ID:AF189262|acc:AF189262.1) a closely related Rattus norvegicus GABA-A receptor epsilon-like subunit (Epsilon) mRNA, complete cds homolog in species Rattus norvegicus Adipose, Peripheral Blood, Spinal Chord, Testis, Colon. This information was derived by determining the tissue sources of the sequences that were included in the invention including but not limited to SeqCalling sources, Public EST sources, Literature sources, and/or RACE sources.

The disclosed NOV51 polypeptide has homology to the amino acid sequences shown in the BLASTP data listed in Table 51C.

TABLE 51C
BLAST results for NOV51

| Gene Index/ | | Length | Identity | Positives | |
|---|---|---|---|---|---|
| Identifier | Protein/Organism | (aa) | (%) | (%) | Expect |
| gi\|16418457\|ref\|NP_{—} | solute carrier | 970 | 394/396 | 394/396 | 0.0 |
| 443193.1\| | family 26, member | | (99%) | (99%) | |
| (NM_052961 | 8 [Homosapiens] | | | | |
| gi\|16588684\|gb\|AAL2 | anion | 970 | 405/464 | 418/464 | 0.0 |
| 6868.1\|AF314959_1 | transporter/ | | (87%) | (89%) | |
| (AF314959) | exchanger-8 [Homo | | | | |
| | sapiens] | | | | |
| gi\|16552904\|dbj\|BAB | unnamed protein | 278 | 171/182 | 172/182 | e−92 |
| 71408.1\|(AK057276) | product [Homo | | (93%) | (93%) | |
| | sapiens] | | | | |
| gi\|9453726\|emb\|CAB9 | bA48209.2 (Novel | 123 | 123/123 | 123/123 | 3e−62 |
| 9352.1\|(AL133507) | sulphate | | (100%) | (100%) | |
| | transporter | | | | |
| | family member) | | | | |
| | [Homo sapiens] | | | | |
| gi\|15080864\|gb\|AAK5 | chloride-formate | 734 | 78/251 | 142/251 | e−35 |
| 1131.1\|(AY032863 | exchanger [Mus | | (31%) | (56%) | |
| | musculus] | | | | |

Tables 51D-E list the domain descriptions from DOMAIN analysis results against NOV51. This indicates that the NOV51 sequence has properties similar to those of other proteins known to contain this domain.

TABLE 51D
Domain Analysis of NOV51

gnl|Pfam|pfam00916, Sulfate_transp, Sulfate transporter family.
Mutations in human Diastrophic Dysplasia Protein lead to several diseases.
CD-Length = 312 residues, 79.5% aligned
Score = 145 bits (366), Expect = 7e−36

TABLE 51E
Domain Analysis of NOV51

gnl|Pfam|pfam01740, STAS, STAS domain. The STAS (after Sulphate
Transporter and AntiSigma factor antagonist) domain is found in the C
terminal region of Sulphate transporters and bacterial antisigma
factor antagonists. It has been suggested that this domain may have a
general NTP binding function.
CD-Length = 106 residues, 63.2% aligned
Score = 53.1 bits (126), Expect = 5e−08

The disclosed NOV51 nucleic acid of the invention encoding a sulfate transporter-like protein includes the nucleic acid whose sequence is provided in Table 51A or a fragment thereof. The invention also includes a mutant or variant nucleic acid any of whose bases may be changed from the corresponding base shown in Table 51A while still encoding a protein that maintains its sulfate transporter-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36 percent of the bases may be so changed.

The disclosed NOV51 protein of the invention includes the sulfate transporter-like protein whose sequence is provided in Table 51B. The invention also includes a mutant or variant protein any of whose residues may be changed from the corresponding residue shown in Table 51B while still encoding a protein that maintains its sulfate transporter-like activities and physiological functions, or a functional fragment thereof. In the mutant or variant protein, up to about 0 percent of the residues may be so changed.

The invention further encompasses antibodies and antibody fragments, such as F_{ab} or (F_{ab})₂, that bind immunospecifically to any of the proteins of the invention.

The above defined information for this invention suggests that this sulfate transporter-like protein (NOV51) may function as a member of a “sulfate transporter family”. Therefore, the NOV51 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV51 nucleic acids and proteins of the invention are useful in potential therapeutic applications implicated in diseases including but not limited to various pathologies and disorders as indicated below. For example, a cDNA encoding the sulfate transporter-like protein (NOV51) may be useful in gene therapy, and the sulfate transporter-like protein (NOV51) may be useful when administered to a subject in need thereof. By way of nonlimiting example, the compositions of the present invention will have efficacy for treatment of patients suffering from diastrophic dysplasia and certain other skeletal dysplasias, and adenoma, familial chloride diarrhea, CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV51 nucleic acid encoding the sulfate transporter-like protein of the invention, or fragments thereof, may further be useful in diagnostic applications, wherein the presence or amount of the nucleic acid or the protein are to be assessed.

NOV51 nucleic acids and polypeptides are further useful in the generation of antibodies that bind immuno-specifically to the novel NOV51 substances for use in therapeutic or diagnostic methods. These antibodies may be generated according to methods known in the art, using prediction from hydrophobicity charts, as described in the “Anti-NOVX Antibodies” section below. The disclosed NOV51 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV52

A disclosed NOV52 nucleic acid of 2210 nucleotides (also referred to as CG57748-01) encoding a N-acetylgalactosaminyltransferase-like protein is shown in Table 52A. The start and stop codons are in bold letters.

TABLE 52A
NOV52 nucleotide sequence.

| | |
|---|---|
| TGCAGTTGGCGGGCGCATGTGGGGGCGCACGGCGCGGCGGCGCTGCCCGCGGGAACTGCG | (SEQ ID NO:121) |
| GCGCGGCCGGGAGGCGCTGTTGGTGCTCCTGGCGCTACTGGCGTTGGCCGGGCTGGGCTC | |
| GGTGCTGCGGGCGCAGCGTGGGGCCGGGGCCGGGGCTGCCGAGCCGGGACCCCCGCGCAC | |
| CCCGCGCCCCGGGCGGCGCGAGCCGGTCATGCCGCGGCCGCCGGTGCCGGCGAACGCGCT | |
| GGGCGCGCGGGGCGAGGCGGTGCGGCTGCAGCTGCAGGGCGAGGAGCTGCGGCTGCAGGA | |
| GGAGAGCGTGCGGCTGCACCAGATTAACATCTACCTCAGCGACCGCATCTCACTGCACCG | |
| CCGCCTGCCCGAGCGCTGGAACCCGCTGTGCAAGAGAAGAAATATGATTATGATAAATTT | |
| GCCCAGGACATCTGTTATCATAGCATTTTATAATGAAGCCTGGTCAACTCTCCTTCGGAC | |
| AGTTTACAGTGTCCTTGAGACATCCCCGGATATCCTGCTAGAAGAAGTGATCCTTGTAGA | |
| TGACTACAGTGATAGAGAGCACCTGAAGGAGCGCTTGGCCAATGAGCTTTCGGGACTGCC | |
| CAAGGTGCGCCTGATCCGCGCCAACAAGAGAGAGGGCCTGGTGCGAGCCCGGCTGCTGGG | |
| GGCGTCTGCGGCGAGGGGCGATGTTCTGACCTTCCTGGACTGTCACTGTGAGTGCCACGA | |
| AGGGTGGCTGGAGCCGCTGCTGCAGAGGATCCATGAAGAGGAGTCGGCAGTGGTGTGCCC | |
| GGTGATTGATGTGATCGACTGGAACACCTTCGAATACCTGGGGAACTCCGGGGAGCCCCA | |
| GATCGGCGGTTTCGACTGGAGGCTGGTGTTCACGTGGCACACAGTTCCTGAGAGGGAGAG | |
| GATACGGATGCAATCCCCCGTCGATGTCATCAGGTCTCCAACAATGGCTGGTGGGCTGTT | |
| TGCTGTGAGTAAGAAATATTTTGAATATCTGGGGTCTTATGATACAGGAATGGAAGTTTG | |
| GGGAGGAGAAAACCTCGAATTTTCCTTTAGGATCTGGCAGTGTGGTGGGGTTCTGGAAAC | |
| ACACCCATGTTCCCATGTTGGCCATGTTTTCCCCAAGCAAGCTCCCTACTCCCGCAACAA | |
| GGCTCTGGCCAACAGTGTTCGTGCAGCTGAAGTATGGATGGATGAATTTAAAGAGCTCTA | |
| CTACCATCGCAACCCCCGTGCCCGCTTGGAACCTTTTGGGGATGTGACAGAGAGGAAGCA | |
| GCTCCGGGACAAGCTCCAGTGTAAAGACTTCAAGTGGTTCTTGGAGACTGTGTATCCAGA | |
| ACTGCATGTGCCTGAGGACAGGCCTGGCTTCTTCGGGATGCTCCAGAACAAAGGACTAAC | |
| AGACTACTGCTTTGACTATAACCCTCCCGATGAAAACCAGATTGTGGGACACCAGGTCAT | |
| TCTGTACCTCTGTCATGGGATGGGCCAGAATCAGTTTTTCGAGTACACGTCCCAGAAAGA | |
| AATACGCTATAACACCCACCAGCCTGAGGGCTGCATTGCTGTGGAAGCAGGAATGGATAC | |
| CCTTATCATGCATCTCTGCGAAGAAACTGCCCCAGAGAATCAGAAGTTCATCTTGCAGGA | |
| GGATGGATCTTTATTTCACGAACAGTCCAAGAAATGTGTCCAGGCTGCGAGGAAGGAGTC | |
| GAGTGACAGTTTCGTTCCACTCTTACGAGACTGCACCAACTCGGATCATCAGAAATGGTT | |
| CTTCAAAGAGCGCATGTTATGAAGCCTCGTGTATCAAGGAGCCCCATCGAAGGAGACTGT | |
| GGAGCCAGGACTCTGCCCAACAAAGACTTAGCTAAGCAGTGACCAGAACCACAAAAACTA | |
| GGCTGGATTGCTTTTGCAAGAGGCAATCATTTGCCCTTTGTGAAAGTGTGTGGATTAGGT | |
| AACAGTGATAGCTGTACTATTTGGCACCTTCTAATGTTCAAATACCTATTTCCAGGTACT | |
| CAGATGGTACCCTGTTTTTGAATTAACCTTTAATTTTCTTCAAACGTATTTAACACGCGG | |
| CCTAACTTCTAGACAAGAAAGATCTTCGGGGGTCACAACCCCCGAAGAATTCGGCGGACC | |
| GTCCACCCTGCTACTAGTCACCCGCGGAGCCAACAACGCCAAGCGCTGCATCACACTCTA | |
| GCACGGCGGCCCACACGAACACATCAAGCAGAGGCAGATACCATAATAGT | |

In a search of public sequence databases, the NOV52 nucleic acid sequence, located on chromsome has 951 of 1468 bases (64%) identical to a gb:GENBANK-ID:MMU73819|acc:U73819.1 mRNA from Mus musculus (Mus musculus polypeptide GalNAc transferase-T4 (ppGaNTase-T4) mRNA, complete cds). Public nucleotide databases include all GenBank databases and the GeneSeq patent database.

The disclosed NOV52 polypeptide (SEQ ID NO:122) encoded by SEQ ID NO:121 has 581 amino acid residues and is presented in Table 52B using the one-letter amino acid code. Signal P, Psort and/or Hydropathy results predict that NOV52 has a signal peptide and is likely to be localized extracellularly with a certainty of 0.8200. The most likely cleavage site for a NOV52 peptide is between amino acids 39 and 40.

TABLE 52B
Encoded NOV52 protein sequence.

| | |
|---|---|
| MWGRTARRRCPRELRRGREALLVLLALLALAGLGSVLRAQRGAGAGAAEPGPPRTPRPGR | (SEQ ID NO:122) |
| REPVMPRPPVPANALGARGEAVRLQLQGEELRLQEESVRLHQINIYLSDRISLHRRLPER | |
| WNPLCKEKKYDYDNLPRTSVIIAFYNEAWSTLLRTVYSVLETSPDILLEEVILVDDYSDR | |
| EHLKERLANELSGLPKVRLIRANKREGLVRARLLGASAARGDVLTFLDCHCECHEGWLEP | |
| LLQRIHEEESAVVCPVIDVIDWNTFEYLGNSGEPQIGGFDWRLVFTWHTVPERERIRMQS | |
| PVDVIRSPTMAGGLFAVSKKYFEYLGSYDTGMEVWGGENLEFSFRIWQCGGVLETHPCSH | |
| VGHVFPKQAPYSRNKALANSVRAAEVWMDEFKELYYHRNPRARLEPFGDVTERKQLRDKL | |
| QCKDFKWFLETVYPELHVPEDRPGFFGMLQNKGLTDYCFDYNPPDENQIVGHQVILYLCH | |
| GMGQNQFFEYTSQKEIRYMTHQPEGCIAVEAGMDTLIMHLCEETAPENQKFILQEDGSLF | |
| HEQSKKCVQAARKESSDSFVPLLRDCTNSDHQKWFFKERML | |

A search of sequence databases reveals that the NOV52 amino acid sequence has 330 of 566 amino acid residues (58%) identical to, and 408 of 566 amino acid residues (72%) similar to, the 578 amino acid residue ptnr:SPTREMBL-ACC:O08832 protein from Mus musculus (Mouse) (POLYPEPTIDE GALNAC TRANSFERASE-T4). Public amino acid databases include the GenBank databases, SwissProt, PDB and PIR.

NOV52 is expressed in at least bone marrow, colon, lung, ovary, kidney, respiratory bronchiole, stomach, testis, tonsils, and germ cells. Expression information was derived from the tissue sources of the sequences that were included in the derivation of the sequence of CG57748-01. The sequence is predicted to be expressed in the following tissues because of the expression pattern of (GENBANK-ID: gb:GENBANK-ID:MMU73819|acc:U73819.1) a closely related Mus musculus polypeptide GalNAc transferase-T4 (ppGaNTase-T4) mRNA, complete cds homolog in species Mus musculus: spleen. This information was derived by determining the tissue sources of the sequences that were included in the invention including but not limited to SeqCalling sources, Public EST sources, Literature Sources, and/or RACE sources.

The disclosed NOV52 polypeptide has homology to the amino acid sequences shown in the BLASTP data listed in Table 52C.

TABLE 52C
BLAST results for NOV52

| Gene Index/ | | Length | Identity | Positives | |
|---|---|---|---|---|---|
| Identifier | Protein/Organism | (aa) | (%) | (%) | Expect |
| gi\|7657112\|ref\|NP_{—} | UDP-N-acetyl- | 578 | 330/570 | 408/570 | e−180 |
| 056552.1\| | alpha-D- | | (57%) | (70%) | |
| (NM_015737) | galactosamine: | | | | |
| | polypeptide N- | | | | |
| | acetylgalactos- | | | | |
| | aminyltransferase 4; | | | | |
| | ppGaNTase-T4 [Mus | | | | |
| | musculus] | | | | |
| gi\|4503901\|ref\|NP_{—} | polypeptide N- | 578 | 324/570 | 402/570 | e−179 |
| 003765.1\| | acetylgalactos- | | (56%) | (69%) | |
| (NM_003774 | aminyltransferase 4 | | | | |
| | UDP-N-acetyl- | | | | |
| | alpha-D- | | | | |
| | galactosamine: | | | | |
| | polypeptide N- | | | | |
| | acetylgalactos- | | | | |
| | aminyltransferase 4; | | | | |
| | GalNAc transferase | | | | |
| | 4; UDP-GalNAc: | | | | |
| | polypeptide N- | | | | |
| | acetylgalactos- | | | | |
| | aminyltransferase 4 | | | | |
| gi\|13375881\|ref\|NP_{—} | protein FLJ21212 | 284 | 284/284 | 284/284 | e−174 |
| 078918.1\| | [Homo sapiens] | | (100%) | (100%) | |
| (NM_024642) | | | | | |
| gi\|15530299\|gb\|AAH | Similar to hypothetical | 272 | 272/272 | 272/272 | e−166 |
| 13945.1\|AAH13945 | protein FLJ21212\|Homo | | (100%) | (100%) | |
| (BC013945) | sapiens] | | | | |
| gi\|14530626\|emb\|CA | cDNA EST | 623 | 248/529 | 322/529 | e−128 |
| C42368.1\| | EMBL:AF031835 | | (46%) | (59%) | |
| (AL110487) | comes from this gene | | | | |
| | [Caenorhabditis | | | | |
| | elegans] | | | | |

This indicates that the NOV52 sequence has properties similar to those of other proteins known to contain this domain.

TABLE 52D
Domain Analysis of NOV52

gnl|Pfam|pfam00535, Glycos_transf_2, Glycosyl transferase. Diverse
family, transferring sugar from UDP-glucose, UDP-N-acetyl-
galactosamine, GDP-mannose or CDP-abequose, to a range of substrates
including cellulose, dolichol phosphate and teichoic acids.
CD-Length = 168 residues, 100.0% aligned
Score = 109 bits (273), Expect = 4e−25

TABLE 52E
Domain Analysis of NOV52

gnl|Smart|smart00458, RICIN, Ricin-type beta-trefoil; Carbohydrate-
binding domain formed from presumed gene triplication
CD-Length = 125 residues, 97.6% aligned
Score = 78.2 bits (191), Expect = 1e−15

NOV52 has homology with uridine diphosphate (UDP)-GalNAc: polypeptide N-acetylgalactosaminyltransferase (GalNAc transferase), a member of the glycosyl transferase family. This enzyme catalyzes the initial step in mucin-type O-glycosylation of specific proteins. Glycosylation of cell surface proteins is critical to normal development, immune response and tissue functions, as evidenced by the phenotypes of a number of mouse knockout models (See Muramatsu; J Biochem (Tokyo) 2000 February;127(2):171-6). Glycosylation patterns are known to change during the process of carcinogenesis (Kohsaki et al., J Gastroeniterol 2000;35(11):840-8). Alterations of these patterns by introducing a transgene coding for a GalNAc transferase (See Tsurifune et al., Int J Oncol 2000 July;17(1):159-65) or by means of antisense oligonucleotides (See Zeng et al., Proc Natl Acad Sci USA Sep. 12, 1995;92(19):8670-4) alter cell morphology, growth and adhesion patterns. Therefore these proteins are important markers and therapeutic targets for oncology applications. In addition, a member of this family has been implicated in autosomal dominant hypophosphatemic rickets (See White et al., Gene Apr. 4, 2000;246(1-2):347-56).

Glycosyl transferases comprise a fairly diverse group of proteins that catalyze the addition of sugar from UDP-glucose, UDP-N-acetyl-galactosamine, GDP-mannose or CDP-abequose, to a range of substrates including cellulose, dolichol phosphate and teichoic acids.

The disclosed NOV52 nucleic acid of the invention encoding a N-acetylgalactosaminyltransferase-like protein includes the nucleic acid whose sequence is provided in Table 52A or a fragment thereof. The invention also includes a mutant or variant nucleic acid any of whose bases may be changed from the corresponding base shown in Table 52A while still encoding a protein that maintains its N-acetylgalactosaminyltransferase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36 percent of the bases may be so changed.

The disclosed NOV52 protein of the invention includes the N-acetylgalactosaminyltransferase-like protein whose sequence is provided in Table 52B. The invention also includes a mutant or variant protein any of whose residues may be changed from the corresponding residue shown in Table 52B while still encoding a protein that maintains its N-acetylgalactosaminyltransferase-like activities and physiological functions, or a functional fragment thereof. In the mutant or variant protein, up to about 42 percent of the residues may be so changed.

The invention further encompasses antibodies and antibody fragments, such as F_{ab} or (F_{ab})₂, that bind immunospecifically to any of the proteins of the invention.

The above defined information for this invention suggests that this N-acetylgalactosaminyltransferase-like protein (NOV52) may function as a member of a “N-acetylgalactosaminyltransferase family”. Therefore, the NOV52 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV52 nucleic acids and proteins of the invention are useful in potential therapeutic applications implicated in diseases including but not limited to various pathologies and disorders as indicated below. For example, a cDNA encoding the N-acetylgalactosaminyltransferase-like protein (NOV52) may be useful in gene therapy, and the N-acetylgalactosaminyltransferase-like protein (NOV52) may be useful when administered to a subject in need thereof. By way of nonlimiting example, the compositions of the present invention will have efficacy for treatment of patients suffering from hemophilia, hypercoagulation, idiopathic thrombocytopenic purpura, immunodeficiencies, graft versus host disease, hypercoagulation, autoimmune disease, allergics, transplantation, Hirschsprung's disease, Crohn's Disease, appendicitis, systemic lupus erythematosus, autoimmune disease, asthma, emphysema, scleroderma, allergy, ARDS, endometriosis, fertility, diabetes, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, hypercalceimia, ulcers, fertility, hypogonadism, polycystic ovarian syndrome, cancer, tissue degeneration, bacterial/viral/parasitic infection, or other pathologies or conditions. The NOV52 nucleic acid encoding the N-acetylgalactosaminyltransferase-like protein of the invention, or fragments thereof, may further be useful in diagnostic applications, wherein the presence or amount of the nucleic acid or the protein are to be assessed.

NOV52 nucleic acids and polypeptides are further useful in the generation of antibodies that bind immuno-specifically to the novel NOV52 substances for use in therapeutic or diagnostic methods. These antibodies may be generated according to methods known in the art, using prediction from hydrophobicity charts, as described in the “Anti-NOVX Antibodies” section below. The disclosed NOV52 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV53

A disclosed NOV53 nucleic acid of 2030 nucleotides (also referred to as CG57693-01) encoding a protein kinase-like protein is shown in Table 53A. The start and stop codons are in bold letters.

TABLE 53A
NOV53 nucleotide sequence.

| | |
|---|---|
| TTTTTTTTTTTTTTGACAATCACCCAGTCAGTATTTATTAGGCGCCTACTGCGGACGGTT | (SEQ ID NO:123) |
| GGTCTTCACGCAGATCAGGCGAGAAGGGATAGTGATGCGCGGGCCGCTGCGGAGACCCGG | |
| AGCCCGCCGCGGATCACGGGAATTTCGCGCCTATTTTTTGTTGGCTGGGTGTTCTCGCCA | |
| GTGATTGGGTCCCGGCAAGGCGTGCCGGTGCGCTCGGCTGCGGCTGCTCCGTCGACCTTT | |
| GCAGGACCGGGCGGTGCAGGGCTCACTCGGCTGGCGTCCCGGGGGATGGGCCACCAGGAG | |
| TCTCCGCTGGCCCGGGCGCCGGCGGGAGGTGCAGCTTATGTAAAGAGGTTATGTAAAGGG | |
| CTCAGCTGGCGCGAACACGTGGAAAGCCACGGGAGCCTAGGAGCCCAGGCTTCCCCAGCG | |
| AGCGCCGCGGCAGCAGAAGGATCCGCTACACGCCGGGCTCGGGCCGCCACCTCCCGCGCT | |
| GCTCGGTCCCGGAGGCAGCCCGGGCCCGGAGCGGACCATCCCCAGGCAGGGGCTCCAGGG | |
| GGGAAACGGGCCGCCCGGAAGTGGAGGTGCGCGGGCCAGGTCACAATCCAAGGTCCGGCT | |
| CCTCCGCGTCCCAGGGCCGGACGGAGGGATGAGGCAGGGGGGGCCCGGGCAGCGCCGTTG | |
| CTGCTCCCCCCGCCGCCCGCAGCCATGGAAACGGGGAAGGACGGCGCCCGCAGAGGTACA | |
| CAAAGCCCGGAGCGGAAAAGGCGAAGCCCAGTGCCGCGGGCGCCCAGCACGAAGCTGAGG | |
| CCGGCGGCGGCGGCCCGGGCCATGGATCCGGTGGCGGCCGAGGCCCCGGGCGAGGCCTTC | |
| CTGGCGCGGCGACGGCCTGAGGGCGGTGGCGGGTCCGCGCGGCCGCGTTACAGCCTGTTG | |
| GCGGAGATCGGGCGCGGCAGCTACGGCGTGGTTTATGAGGCAGTGGCCGGGCGCAGCGGG | |
| GCCCGGGTGGCGGTCAAGAAGATCCGCTGCGACGCCCCCGAGAACGTGGAGCTGGCGCTG | |
| GCTGAATTCTGGGCCCTCACCAGCCTCAAGCGGCGCCACCAGAACGTCGTGCAGTTTGAG | |
| GAGTGCGTCCTGCAGCGCAATGGGTTAGCCCAGCGCATGAGTCACGGCAACAAGAGCTCG | |
| CAGCTTTACCTGCGCCTGGTGGAGACCTCGCTGAAAGAAAGGATCCTGGGTTATGCTGAG | |
| GAGCCCTGCTATCTCTGGTTTGTCATGGAGTTCTGTGAAGGTGGAGACCTGAATCAGTAT | |
| GTCCTGTCCCGGAGGCCAGACCCAGCCACCAACAAAAGTTTCATGCTACAGCTGACGAGC | |
| GCCATTGCCTTCCTGCACAAAAACCATATTGTGCACAGGGACCTGAAGCCAGACAACATC | |
| CTCATCACAGAGCGGTCTGGCACCCCCATCCTCAAAGTGGCCGACTTTGGACTAAGCAAG | |
| GTCTGTGCTGGGCTGGCACCCCGAGGCAAAGAGGGCAATCAAGACAACAAAAATGTGAAT | |
| GTGAATAAGTACTGGCTGTCCTCAGCCTGCGGTTCGGACTTCTACATGGCTCCTGAAGTC | |
| TGGGAGGGACACTACACAGCCAAGGCGGACATCTTTGCCCTGGGCATTATCATCTGGGCA | |
| ATGATAGAAAGAATCACTTTTATTGACTCTGAGACCAAGAAGGAGCTCCTGGGGACCTAC | |
| ATTAAACAGGGGACTGAGATCGTCCCTGTTGGTGAGGCGCTGCTAGAAAACCCAAAGATG | |
| GAGTTGCACATCCCCCAAAAACGCAGGACTTCCATGTCTGAGGGGATCAAGCAGCTCTTG | |
| AAAGATATGTTAGCTGCTAACCCACAGGACCGGCCTGATGCCTTTGAACTTGAAACCAGA | |
| ATGGACCAGGTCACATGTGCTGCTTAAAATTCAGGGCTAAGCATTTTGGGTGATTTTAAA | |
| CTAGGTCGATTCCTCGGGACCCACAGTCTCACCACGTCTCCTCCAGAGGACGGCAGAGGG | |
| TACAGGTGGTGGCCTGGCCGGTTGGCGATCTCCCGACAGCTCGATCCGGC | |

In a search of public sequence databases, the NOV53 nucleic acid sequence, located on chromsome 20 has 262 of 361 bases (72%) identical to a gb:GENBANK-ID:AB041802|acc:AB041802.1 mRNA from Mus musculus (Mus musculus brain cDNA, clone MNCb-1723). Public nucleotide databases include all GenBank databases and the GeneSeq patent database.

The disclosed NOV53 polypeptide (SEQ ID NO:124) encoded by SEQ ID NO:123 has 533 amino acid residues and is presented in Table 53B using the one-letter amino acid code. Signal P, Psort and/or Hydropathy results predict that NOV53 has no signal peptide and is likely to be localized to the cytoplasm with a certainty of 0.8500.

TABLE 53B
Encoded NOV53 protein sequence.

| | |
|---|---|
| MGHQESPLARAPAGGAAYVKRLCKGLSWREHVESHGSLGAQASPASAAAAEGSATRRARA | (SEQ ID NO:124) |
| ATSRAARSRRQPGPGADHPQAGAPGGKRAARKWRCAGQVTIQGPAPPRPRAGRRDEAGGA | |
| RAAPLLLPPPPAAMETGKDGARRGTQSPERKRRSPVPRAPSTKLRPAAAARAMDPVAAEA | |
| PGEAFLARRRPEGGGGSARPRYSLLAEIGRGSYGVVYEAVAGRSGARVAVKKIRCDAPEN | |
| VELALAEFWALTSLKRRHQNVVQFEECVLQRNGLAQRMSHGNKSSQLYLRLVETSLKERI | |
| LGYAEEPCYLWFVMEFCEGGDLNQYVLSRRPDPATNKSFMLQLTSAIAFLHKNHIVHRDL | |
| KPDNILITERSGTPILKVADFGLSKVCAGLAPRGKEGNQDNKNVNVNKYWLSSACGSDFY | |
| MAPEVWEGHYTAKADTFALGIIIWAMIERITFIDSETKKELLGTYIKQGTEIVPVGEALL | |
| ENPKMELHIPQKRRTSMSEGIKQLLKDMLAANPQDRPDAFELETRMDQVTCAA | |

A search of sequence databases reveals that the NOV53 amino acid sequence has 517 of 517 amino acid residues (100%) identical to, and 517 of 517 amino acid residues (100%) similar to, the 517 amino acid residue ptnr:TREMBLNEW-ACC:CAC10518 protein from Homo sapiens (Human) (BA55008.2 (NOVEL PROTEIN KINASE)). Public amino acid databases include the GenBank databases, SwissProt, PDB and PIR.

NOV53 is expressed in at least Adrenal Gland/Suprarenal gland, Lymphoid tissue, Oviduct/Uterine Tube/Fallopian tube, Peripheral Blood, Placenta, Retina, Thymus. This information was derived by determining the tissue sources of the sequences that were included in the invention including but not limited to SeqCalling sources, Public EST sources, Literature sources, and/or RACE sources.

The disclosed NOV53 polypeptide has homology to the amino acid sequences shown in the BLASTP data listed in Table 53C.

TABLE 53C
BLAST results for NOV53

| Gene Index/ | | Length | Identity | Positives | |
|---|---|---|---|---|---|
| Identifier | Protein/Organism | (aa) | (%) | (%) | Expect |
| gi\|12830335\|emb\|CAC | bA550O8.2 (novel | 517 | 517/517 | 517/517 | 0.0 |
| 10518.2\|(AL359916) | protein kinase) | | (100%) | (100%) | |
| | [Homo sapiens] | | | | |
| gi\|18592261\|ref\|XP_{—} | serine/threonine | 401 | 400/401 | 400/401 | 0.0 |
| 086681.1\| | kinase 35 [Homo | | (99%) | (99%) | |
| (XM_086681) | sapiens] | | | | |
| gi\|16878290\|gb\|AAH1 | (protein for | 398 | 395/398 | 395/398 | 0.0 |
| 7340.1\|AAH17340 | IMAGE: 4869353) | | (99%) | (99%) | |
| (BC017340) | [Homo sapiens] | | | | |
| gi\|18549074\|ref\|XP_{—} | similar to Cell | 161 | 101/156 | 129/156 | e−56 |
| 086530.1\| | division control | | (64%) | (81%) | |
| (XM_086530) | protein 2 homolog | | | | |
| | (P34 protein | | | | |
| | kinase) [Homo | | | | |
| | sapiens] | | | | |
| gi\|15224378\|ref\|NP_{—} | putative protein | 257 | 92/314 | 143/314 | e−23 |
| 181320.1\| | kinase | | (29%) | (45%) | |
| (NM_129340) | [Arabidopsis | | | | |
| | thaliana] | | | | |

Tables 53D-F list the domain descriptions from DOMAIN analysis results against NOV53. This indicates that the NOV53 sequence has properties similar to those of other proteins known to contain this domain.

TABLE 53D
Domain Analysis of NOV53

gnl|Smart|smart00220, S_TKc, Serine/Threonine protein kinases,
catalytic domain; Phosphotransferases. Serine or threonine-specific
kinase subfamily.
CD-Length = 256 residues, 97.7% aligned
Score = 193 bits (491), Expect = 2e−50

TABLE 53E
Domain Analysis of NOV53

gnl|Pfam|pfam00069, pkinase, Protein kinase domain
CD-Length = 256 residues, 97.7% aligned
Score = 189 bits (481), Expect = 3e−49

TABLE 53F
Domain Analysis of NOV53

gnl|Smart|smart00219, TyrKc, Tyrosine kinase, catalytic domain;
Phosphotransferases. Tyrosine-specific kinase subfamily
CD-Length = 258 residues, 99.6% aligned
Score = 121 bits (303), Expect = 1e−28

Protein phosphorylation is a fundamental process for the regulation of cellular functions. The coordinated action of both protein kinases and phosphatases controls the levels of phosphorylation and, hence, the activity of specific target proteins. One of the predominant roles of protein phosphorylation is in signal transduction, where extracellular signals are amplified and propagated by a cascade of protein phosphorylation and dephosphorylation events. Eukaryotic protein kinases are enzymes that belong to a very extensive family of proteins which share a conserved catalytic core common with both serine/threonine and tyrosine protein kinases. There are a number of conserved regions in the catalytic domain of protein kinases. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme. Protein kinases are excellent small molecule drug targets for therapeutic intervention.

The disclosed NOV53 nucleic acid of the invention encoding a protein kinase-like protein includes the nucleic acid whose sequence is provided in Table 53A or a fragment thereof. The invention also includes a mutant or variant nucleic acid any of whose bases may be changed from the corresponding base shown in Table 53A while still encoding a protein that maintains its protein kinase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 28 percent of the bases may be so changed.

The disclosed NOV53 protein of the invention includes the protein kinase-like protein whose sequence is provided in Table 53B. The invention also includes a mutant or variant protein any of whose residues may be changed from the corresponding residue shown in Table 53B while still encoding a protein that maintains its protein kinase-like activities and physiological functions, or a functional fragment thereof. In the mutant or variant protein, up to about 0 percent of the residues may be so changed.

The invention further encompasses antibodies and antibody fragments, such as F_{ab} or (F_{ab})₂, that bind immunospecifically to any of the proteins of the invention.

The above defined information for this invention suggests that this protein kinase-like protein (NOV53) may function as a member of a “protein kinase family”. Therefore, the NOV53 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV53 nucleic acids and proteins of the invention are useful in potential therapeutic applications implicated in diseases including but not limited to various pathologies and disorders as indicated below. For example, a cDNA encoding the protein kinase-like protein (NOV53) may be useful in gene therapy, and the protein kinase-like protein (NOV53) may be useful when administered to a subject in need thereof. By way of nonlimiting example, the compositions of the present invention will have efficacy for treatment of patients suffering from adrenoleukodystrophy, congenital adrenal hyperplasia, anemia, ataxia-telangiectasia, autoimmune disease, fertility, hemophilia, hypercoagulation, idiopathic thrombocytopenic purpura, graft versus host disease, allergies, immunodeficiencies, transplantation, graft versus host disease (GVHD), lymphaedema, Von Hippel-Lindau (VIAL) syndrome, diabetes, tuberous sclerosis, or other pathologies or conditions. The NOV53 nucleic acid encoding the protein kinase-like protein of the invention, or fragments thereof, may further be useful in diagnostic applications, wherein the presence or amount of the nucleic acid or the protein are to be assessed.

NOV53 nucleic acids and polypeptides are further useful in the generation of antibodies that bind immuno-specifically to the novel NOV53 substances for use in therapeutic or diagnostic methods. These antibodies may be generated according to methods known in the art, using prediction from hydrophobicity charts, as described in the “Anti-NOVX Antibodies” section below. The disclosed NOV53 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV54

A disclosed NOV54 nucleic acid of 3331 nucleotides (also referred to as CG57707-01) encoding a Leucine-rich glioma-inactivated protein precursor-like protein is shown in Table 54A. The start and stop codons are in bold letters.

TABLE 54A
NOV54 nucleotide sequence.

| | |
|---|---|
| ATGGCGCTGCGGAGAGGCGGCTGCGGAGCGCTCGGGCTGCTGCTGCTGCTGCTGGGCGCC | (SEQ ID NO:125) |
| GCGTGCCTGATACCGCGGAGCGCGCAGGTGAGGCGGCTGGCGCGCTGCCCCGCCACTTGC | |
| AGCTGTACCAAGGAGTCTATCATCTGCGTGGGCTCTTCCTGGGTGCCCAGGATCGTGCCG | |
| GGCGACATCAGCTCCCTGAGCCTGGTAAATGGGACGTTTTCAGAAATCAAGGACCGAATG | |
| TTTTCCCATCTGCCTTCTCTGCAGCTGCTATTGCTGAATTCTAACTCATTCACGATCATC | |
| CGGGATGATGCTTTTGCTGGACTTTTTCATCTTGAATACCTGTTCATTGAAGGGAACAAA | |
| ATAGAAACCATTTCAAGAAATGCCTTTCGTGGCCTCCGTGACCTGACTCACCTTTCTTTG | |
| GCCAATAACCACATAAAAGCACTACCAAGGGATGTCTTCAGTGATTTAGACTCTCTGATT | |
| GAACAGATTTTGAGGGGTAATAAATTTGAATGTGACTGCAAAGCCAAGTGGCTATACCTG | |
| TGGTTGAAGATGACAAATTCCACCGTTTCTGATGTGCTGTGTATTGGTCCACCAGAGTAT | |
| CAGGAAAAGAAGCTAAATGACGTGACCAGCTTTGACTATGAATGCACAACTACAGATTTT | |
| GTTGTTCATCAGACTTTACCCTACCAGTCGGTTTCAGTGGATACGTTCAACTCCAAGAAC | |
| GATGTGTACGTGGCCATCGCGCAGCCCACCATGGAGAACTGCATGGTGCTGGAGTGGGAC | |
| CACATTGAAATGAATTTCCGGAGCTATGACAACATTACAGGTCAGTCCATCGTGGGCTGT | |
| AAGGCCATTCTCATCGATGATCAGGTCTTTGTGGTGGTAGCCCAGCTCTTCGGTGGCTCT | |
| CACATTTACAAATACGACGAGAGTTGGACCAAATTTGTCAAATTCCAAGACATAGAGGTC | |
| TCTCGCATTTCCAAGCCCAATGACATCGAGCTGTTTCAGATCGACGACGAGACGTTCTTT | |
| GTCATCGCAGACAGCTCAAAGGCTGGTCTGTCCACAGTTTATAAATGGAACAGCAAAGGA | |
| TTCTATTCTTACCAGTCACTGCACGAGTGGTTCAGGGACACCGATGCGGAGTTTGTTGAT | |
| ATCGATGGAAAATCGCATCTCATCCTGTCCAGCCGCTCCCAGGTCCCCATCATCCTCCAG | |
| TGGAATAAAAGCTCTAAGAAGTTTGTCCCCCATGGTGACATCCCCAACATGGAGGACGTA | |
| CTGGCTGTGAAGAGCTTCCGAATGCAAAATACCCTCTACCTTTCCCTTACCCGCTTCATC | |
| GGGGACTCCCGGGTCATGAGGTGGAACAGTAAGCAGTTTGTGGAGATCCAAGCTCTTCCA | |
| TCCCGGGGGGCCATGACCCTGCAGCCCTTTTCTTTTAAAGATAATCACTACCTGGCCCTG | |
| GGGAGTGACTATACATTCTCTCAGATATACCAGTGGGATAAAGAGAAGCAGCTATTCAAA | |
| AAGTTTAAGGAGATTTATGTGCAGCCGCCTCGTTCATTCACAGCTGTCTCCACCGACAGG | |
| AGAGATTTCTTTTTTGCATCCAGTTTCAAAGGGAAAACAAAGATTTTTGAACATATAATT | |
| GTTGACTTAAGTTTGTGAAGGTGTGGTGGGTGAAACTAAGAGAAATGTAGCATTAGCTCT | |
| CACAAAAGAGGACCAAGAAAAATCAACAAACAAATCAAAGCCAGGCTCAGAGCTCTGAAA | |
| TTAAAAAGCACTGAAATAGTTAGATGTTTTCAAACTTTTAGAACTCACATTTTAATCAGG | |
| GATTGCATTTATTGGCTAACTGCATGACATGCCCATTCTACCATTTTAAAAAAAATCTTA | |
| AAGCCTGTAATTTCTGAGAAAAGAGTACAGCATTTACTCTTATCATCTAGAAATGTAATA | |
| TGCTTCCCCCCCGCTTTTTGATGAGGAAGAAGACAATTGGATAAGATGGGACAGCACTTA | |
| TAATGAAATAAAAAAAAACTTTGAGCCCCTCTCATTCCACTTTAGCAATCTTTTTGGTAA | |
| GAACTCTTAAAGCCAAAAGTCTGCTGAAAAGATTTGCTGATTATTAGTTTAAAAATCTTG | |
| TAACACTCAGCAGTGCTATTTTGAGTCATCCCAGTTTCCTGAAAGTAATGCCCAGTCTTC | |
| CTGAATCCTCCTTAATACCAGAACCTTGGTOATTTTGTTGGCTCATATGAATGCTTGTCA | |
| TGGATATGTTAACAATTTAGTGTTTGACATTGCTTCCTCTGCCACAAAGACAATACTCTG | |
| GTGACACATGTCTAGGCCCAGCACAGGCTGTAGGCCCAGGAGTGACTCAAAGGAGTTTTT | |
| CCCTCTTTCTTACGGTTCAAAGGTGACCCTGGTGGTGGCCACAGCAGTAATGCTTGTTTG | |
| ATGCTCTTCATGGCTCATCTGCTTCTCAGAACCCACCCGTTGAGTTTGTGGGTAACCAGC | |
| AGGCAGGCCAAAGACTGGTGCTTTTCATTTCATCCTTTAGAGGGATGAAACAGTTATTTC | |
| CGTCTGATGAGCATTCGGTAGAATTTTTGAAGTGAGATTTTATGAAGTCAAAGGGGACTT | |
| TACACAGATCTCGACCTGCTTTGAAACCTAGAGGTGGCCCTTTGATTTGTGCGTGTCCTT | |
| GCCCTCTGGACAACTTAATATTTCAAGTAATCGAATACCAACTTCCCTGCCAGCCCACCT | |
| GCCTTCCGCCCCGCTTGTGTAACAGTCCTGTTTTGTTGAGTTGCTGCTATTGCACTGCCA | |
| GTGCAGCCCACACCAAATCACAACCCAAGATACTCAGATAGGAAGACTCCTTCCTCTCCC | |
| AGTACTTTACCAAAGGAACCCCCGCCAGGACCCACATGGGGCCACGTGTTGGCAGTGGAA | |
| TCAGCCTGTGCAGGCTGGGGATCTCAGGCTGATCAGTAGGGGCCAGCTTTGGAGCCAGCC | |
| AAGCTGAATCCCACACTCCAGGTCTGTGCTCAAGAGACCAGATGGTGTATTTCCAAATGG | |
| GCCTCTCTGGTATGGGCAATAGGCAAGCTCCTGGGGTCTGGTTATGTGGAAGATTCTTAG | |
| TGGATGTTCCGCCTGGTTAGCTGGTTCTCTTCAGAGAATATAAAGTGAATGCCTTTAGGG | |
| GTAGCTCTGAAAGAGAAACCCAACAACTTCATTCCTAGCCATGAAAGTAGCACGATCATA | |
| TTGTACTGTATTGTTATTGTAAAATGACTATTTGCCATGTCATGAGTAGGTAGATGTTTT | |
| GCCACAAATATGAATGTCTTTGTTGTTTCCTGACTTTAAGCAATGAAGATTGAGACAATA | |
| AATAGCACTCAGAGAATGAAGCATTGATGTT | |

In a search of public sequence databases, the NOV54 nucleic acid sequence, located on chromsome 4p16 has 682 of 1052 bases (64%) identical to a gb:GENBANK-ID:AF055636|acc:AF055636.1 mRNA from Homo sapiens (Homo sapiens leucine-rich glioma-inactivated protein precursor (LGI1) mRNA, complete cds). Public nucleotide databases include all GenBank databases and the GeneSeq patent database.

The disclosed NOV54 polypeptide (SEQ ID NO:126) encoded by SEQ ID NO:125 has 545 amino acid residues and is presented in Table 54B using the one-letter amino acid code. Signal P, Psort and/or Hydropathy results predict that NOV54 has a signal peptide and is likely to be localized localized extracellularly with a certainty of 0.8200. The most likely cleavage site for a NOV54 peptide is between amino acids 22 and 23.

TABLE 54B
Encoded NOV54 protein sequence.

| | |
|---|---|
| MALRRGGCGALGLLLLLLGAACLIPRSAQVRRLARCPATCSCTKESIICVGSSWVPRIVP | (SEQ ID NO:126) |
| GDISSLSLVNGTFSEIKDRMFSHLPSLQLLLLNSNSFTIIRDDAFAGLFHLEYLFIEGNK | |
| IETISRNAFRGLRDLTHLSLANNHIKALPRDVFSDLDSLIEQILRGNKFECDCKAKWLYL | |
| WLKMTNSTVSDVLCIGPPEYQEKKLNDVTSFDYECTTTDFVVHQTLPYQSVSVDTFNSKN | |
| DVYVAIAQPSMENCMVLEWDHIEMNFRSYDNITGQSIVGCKAILIDDQVFVVVAQLFGGS | |
| HIYKYDESWTKFVKFQDIEVSRISKPNDIELFQIDDETFFVIADSSKAGLSTVYKWNSKG | |
| FYSYQSLHEWFRDTDAEFVDIDGKSHLILSSRSQVPIILQWNKSSKKFVPHGDTPNMEDV | |
| LAVKSFRMQNTLYLSLTRFIGDSRVMRWNSKQFVEIQALPSRGAMTLQPFSFKDNHYLAL | |
| GSDYTFSQIYQWDKEKQLFKKFKEIYVQAPRSFTAVSTDRRDFFFASSFKGKTKIFEHII | |
| VDLSL | |

A search of sequence databases reveals that the NOV54 amino acid sequence has 301 of 538 amino acid residues (55%) identical to, and 386 of 538 amino acid residues (71%) similar to, the 557 amino acid residue ptnr:SPTREMBL-ACC:O95970 protein from Homo sapiens (Human) (LEUCINE-RICH GLIOMA-INACTIVATED PROTEIN PRECURSOR). Public amino acid databases include the GenBank databases, SwissProt, PDB and PIR.

NOV54 is expressed in at least Appendix, brain, colon, heart, kidney, ovary, pancreas, parathyroid gland, uterus, and vein. This information was derived by determining the tissue sources of the sequences that were included in the invention including but not limited to SeqCalling sources, Public EST sources, Literature sources, and/or RACE sources.

The disclosed NOV54 polypeptide has homology to the amino acid sequences shown in the BLASTP data listed in Table 54C.

TABLE 54C
BLAST results for NOV54

| Gene Index/ | | Length | Identity | Positives | |
|---|---|---|---|---|---|
| Identifier | Protein/Organism | (aa) | (%) | (%) | Expect |
| gi\|15620891\|dbj\|BAB | KIAA1916 protein | 542 | 539/542 | 539/542 | 0.0 |
| 67809.1\| (AB067503) | [Homo sapiens] | | (99%) | (99%) | |
| gi\|9938002\|ref\|NP_0 | leucine-rich, | 557 | 296/516 | 378/516 | e−178 |
| 64674.1\| | glioma | | (57%) | (72%) | |
| (NM_020278) | inactivated 1 | | | | |
| | [Mus musculus] | | | | |
| gi\|4826816\|ref\|NP_0 | leucine-rich, | 557 | 296/516 | 379/516 | e−178 |
| 05088.1\| | glioma | | (57%) | (73%) | |
| (NM_005097) | inactivated 1 | | | | |
| | precursor [Homo | | | | |
| | sapiens] | | | | |
| gi\|15722102\|emb\|CAC | bA512J3.1 | 461 | 269/460 | 342/460 | e−160 |
| 78757.1\| (AL358154 | (leucine-rich, | | (58%) | (73%) | |
| | glioma | | | | |
| | inactivated 1) | | | | |
| | [Homo sapiens] | | | | |
| gi\|18591028\|ref\|XP_{—} | protein XP_092048 | 466 | 179/437 | 267/437 | 3e−93 |
| 092048.1\| | [Homo sapiens] | | (40%) | (60%) | |
| (XM_092048) | | | | | |

Table 54D lists the domain descriptions from DOMAIN analysis results against NOV54. This indicates that the NOV54 sequence has properties similar to those of other proteins known to contain this domain.

TABLE 54D
Domain Analysis of NOV54

gnl|Pfam|pfam01463, LRRCT, Leucine rich repeat C-terminal domain.
Leucine Rich Repeats pfam00560 are short sequence motifs present in a
number of proteins with diverse functions and cellular locations.
Leucine Rich Repeats are often flanked by cysteine rich domains. This
domain is often found at the C-terminus of tandem leucine rich
repeats.
CD-Length = 51 residues, 98.0% aligned
Score = 43.1 bits (100), Expect = 4e−05

Loss of heterozygosity for 10q23-26 is seen in over 80% of glioblastoma multiforme tumors. Positional cloning was used to isolate the LGI1 (Leucine-rich gene-Glioma Inactivated) gene, which is rearranged as a result of the t(10;19)(q24;q13) balanced translocation in the T98G glioblastoma cell line lacking any normal chromosome 10 (See Chernova et al., Oncogene 17: 2873-2881). Rearrangement of the LGI1 gene was also detected in the A172 glioblastoma cell line and several glioblastoma tumors. These rearrangements lead to a complete absence of LGI1 expression in glioblastoma cells. The LGI1 gene encodes a protein with a calculated molecular mass of 60 kD and contains 3.5 leucine-rich repeats (LRR) with conserved flanking sequences. In the LRR domain, LGI1 has the highest homology with a number of transmembrane and extracellular proteins which function as receptors and adhesion proteins. LGI1 is predominantly expressed in neural tissues, especially in brain; its expression is reduced in low grade brain tumors and it is significantly reduced or absent in malignant gliomas. Its localization to the 10q24 region, and rearrangements or inactivation in malignant brain tumors, suggest that LGI1 is a candidate tumor suppressor gene involved in progression of glial tumors.

The human leucine-rich glioma-inactivated protein precursor-like protein described in this invention is predicted to share the attributes of other family members and is thus implicated in regulation of cell growth and survival as well as cellular metabolism. Like the LGI1 gene, the leucine-rich glioma-inactivated protein precursor-like gene described in this patent is expressed in neural tissues; however, it also appears to be frequently expressed in parathyroid tumors. Therefore, this protein is an attractive target for drug intervention in the treatment of cancer, central nervous system disorders, and metabolic diseases, among others. The leucine-rich glioma-inactivated protein precursor-like gene maps to human chromosome 4p16 and is predicted to encode a secreted protein.

The disclosed NOV54 nucleic acid of the invention encoding a Leucine-rich glioma-inactivated protein precursor-like protein includes the nucleic acid whose sequence is provided in Table 54A or a fragment thereof. The invention also includes a mutant or variant nucleic acid any of whose bases may be changed from the corresponding base shown in Table 54A while still encoding a protein that maintains its Leucine-rich glioma-inactivated protein precursor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36 percent of the bases may be so changed.

The disclosed NOV54 protein of the invention includes the Leucine-rich glioma-inactivated protein precursor-like protein whose sequence is provided in Table 54B. The invention also includes a mutant or variant protein any of whose residues may be changed from the corresponding residue shown in Table 54B while still encoding a protein that maintains its Leucine-rich glioma-inactivated protein precursor-like activities and physiological functions, or a functional fragment thereof. In the mutant or variant protein, up to about 45 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Leucine-rich glioma-inactivated protein precursor-like protein (NOV54) may function as a member of a “Leucine-rich glioma-inactivated protein precursor family”. Therefore, the NOV54 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV54 nucleic acids and proteins of the invention are useful in potential therapeutic applications implicated in diseases including but not limited to various pathologies and disorders as indicated below. For example, a cDNA encoding the Leucine-rich glioma-inactivated protein precursor-like protein (NOV54) may be useful in gene therapy, and the Leucine-rich glioma-inactivated protein precursor-like protein (NOV54) may be useful when administered to a subject in need thereof. By way of nonlimiting example, the compositions of the present invention will have efficacy for treatment of patients suffering from cancer, trauma, bacterial and viral infections, in vitro and in vivo regeneration, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, anemia, bleeding disorders, scleroderma, transplantation, hyperparathyroidism, hypoparathyroidism, diabetes, autoimmune disease, renal artery stenosis, interstitial nephritis, glomerulonephritis, polycystic kidney disease, systemic lupus erythematosus, renal tubular acidosis, IgA nephropathy, Lesch-Nyhan syndrome, Hirschsprung's disease, Crohn's Disease, appendicitis, endometriosis, fertility, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, obesity, transplantation, and pancreatitis, or other pathologies or conditions. The NOV54 nucleic acid encoding the Leucine-rich glioma-inactivated protein precursor-like protein of the invention, or fragments thereof, may further be useful in diagnostic applications, wherein the presence or amount of the nucleic acid or the protein are to be assessed.

NOV54 nucleic acids and polypeptides are further useful in the generation of antibodies that bind immuno-specifically to the novel NOV54 substances for use in therapeutic or diagnostic methods. These antibodies may be generated according to methods known in the art, using prediction from hydrophobicity charts, as described in the “Anti-NOVX Antibodies” section below. The disclosed NOV54 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV55

A disclosed NOV55 nucleic acid of 2886 nucleotides (also referred to as CG57306-01) encoding an anion exchanger-like protein is shown in Table 55A. The start and stop codons are in bold letters.

TABLE 55A
NOV55 nucleotide sequence.

| | |
|---|---|
| ATTCTGTGCAAGCCTCATGGAAATGAAGCTGCCAGGCCAGGAAGGGTTTGAAGCCTCCAG | (SEQ ID NO:127) |
| TGCTCCTAGAAATATTCCTTCAGGGGAGCTGGACAGCAACCCTGACCCTGGCACCGGCCC | |
| CAGCCCTGATGGCCCCTCAGACACAGAGAGCAAGGAACTGGGAGTACCCAAAGACCCTCT | |
| GCTCTTCATTCAGCTGAATGAGCTGCTGGGCTGGCCCCAGGCGCTGGAGTGGAGAGAGAC | |
| AGGCCGATGGGTACTGTTTGAGGAGAAGTTGGAGGTCCCTGCAGGCCGGTGGAGTGCCCC | |
| CCACGTGCCCACCCTGGCACTGCCCAGCCTCCAGAAGCTCCGCAGCCTGCTGGCCGAGGG | |
| CCTTGTACTGCTGGACTGCCCAGCTCAGAGCCTCCTGGAGCTCGTGGAGCAGGTGACCAG | |
| GGTGGACTCGCTGAGCCCAGAGCTGAGAGGGCAGTTGCAGGCCTTGCTGCTGCAGAGACC | |
| CCAGCATTACAACCAGACCACAGGCACCAGGCCCTGCTGGGGTGAGAGCCCCTCCAGAAA | |
| GGCTTCTGACAATGAGGAACCCCCCCTGAGGGACCAGTGTCAGAACCCCCTGAGACAGAA | |
| GCTACCTCCAGGAGCTGAGGCAGGGACTGTGCTGGCAGGGGAGCTGGGCTTCCTGGCACA | |
| GCCACTGGGAGCCTTTGTTCGACTGCGGAACCCTGTGGTACTGGGGTCCCTTACTGAGGT | |
| GTCCCTCCCAAGCAGGTTTTTCTGCCTTCTCCTGGGCCCCTGTATGCTGGGAAAGGGCTA | |
| CCATGAGATGGGACGGGCAGCAGCTGTCCTCCTCAGTGACCCGCATTCCCAGCAATTCCA | |
| GTGGTCAGTTCGTCGGGCCAGCAACCTTCATGACCTTCTGGCAGCCCTGGATGCATTCCT | |
| AGAGGAGGTGACAGTGCTTCCCCCAGGTCGGTGGGACCCAACAGCCCGGATTCCCCCGCC | |
| CAAATGTCTGCCATCTCAGCACAAAAGGACCTCGGCTGAGGACAGGCACCGCCATGGGCC | |
| ACACGCACACAGCCCGGAGTTGCAGCGGACCGGCAGGCTGTTTGGGGGCCTTATCCAGGA | |
| CGTGCGCAGGAAGGTCCCGTGGTACCCCAGCGATTTCTTGGACGCCCTGCATCTCCAGTG | |
| CTTCTCGGCCGTACTCTACATTTACCTGGCCACTGTCACTAATGCCATCACTTTTGGGGG | |
| TCTGCTGGGAGATGCCACTGATGGTGCCCAGGGAGTGCTGGAAAGTTTCCTGGGCACAGC | |
| AGTGGCTGGAGCTGCCTTCTGCCTGATGGCAGGCCAGCCCCTCACCATTCTGAGCAGCAC | |
| GGGGCCAGTGCTGGTCTTTGAGCGCCTGCTCTTCTCTTTCAGCAGAGATTACAGCCTGGA | |
| CTACCTGCCCTTCCGCCTATGGGTGGGCATCTGGGTGGCTACCTTTTGCCTGGTGCTGGT | |
| GGCCACAGAGGCCAGTGTGCTGGTGCGCTACTTCACCCGCTTCACTGAGGAAGGTTTCTG | |
| TGCCCTCATCAGCCTCATCTTCATCTACGATGCTGTGGGCAAAATGCTGAACTTGACCCA | |
| TACCTATCCTATCCAGAAGCCTGGGTCCTCTGCCTACGGGTGCCTCTGCCAATACCCAGG | |
| CCCAGGAGGTAATGAGTCTCAATGGATAAGGACAAGGCCAAAAGACAGAGACGACATTGT | |
| AAGCATGGACTTAGGCCTGATCAATGCATCCTTGCTGCCGCCACCTCAGTGCACCCGGCA | |
| GGGAGCCCACCCTCGTGGCCCTGGCTGTCATACAGTCCCAGACATTGCCTTCTTCTCCCT | |
| TCTCCTCTTCCTTACTTCTTTCTTCTTTGCTATGGCCCTCAAGTGTGTAAAGACCAGCCG | |
| CTTCTTCCCCTCTGTGGTGCGCAAAGGGCTCAGCGACTTCTCCTCAGTCCTGGCCATCCT | |
| GCTCGGCTGTGGCCTTGATGCTTTCCTGGGCCTAGCCACACCAAAGCTCATGGTACCCAG | |
| AGAGTTCAAGCCCACACTCCCTGGGCGTGGCTGGCTGGTGTCACCTTTTGGAGCCAACCC | |
| CTGGTGGTGGAGTGTGGCAGCTGCCCTGCCTGCCCTGCTGCTGTCTATCCTCATCTTCAT | |
| GGACCAACAGATCACAGCAGTCATCCTCAACCGCATGGAATACAGACTGCAGAAGGGAGC | |
| TGGCTTCCACCTGGACCTCTTCTGTGTGGCTGTGCTGATGCTACTCACATCACCGCTTGG | |
| ACTGCCTTGGTATGTCTCAGCCACTGTCATCTCCCTGGCTCACATGGACAGTCTTCGGAG | |
| AGAGAGCAGAGCCTGTGCCCCCGGGGAGCGCCCCAACTTCCTGGGTATCAGGGAACAGAG | |
| GCTGACAGGCCTGGTGGTGTTCATCCTTACAGGAGCCTCCATCTTCCTGGCACCTGTGCT | |
| CAAGTTCATTCCAATGCCTGTGCTCTATGGCATCTTCCTGTATATGGGGGTGGCAGCGCT | |
| CAGCAGCATTCAGTTCACTAATAGGGTGAAGCTGTTGTTGATGCCAGCAAAACACCAGCC | |
| AGACCTGCTACTCTTGCGGCATGTGCCTCTGACCAGGGTCCACCTCTTCACAGCCATCCA | |
| GCTTGCCTGTCTGGGGCTGCTTTGGATAATCAAGTCTACCCCTGCAGCCATCATCTTCCC | |
| CCTCATGTTGCTGGGCCTTGTGGGGGTCCGAAAGGCCCTGGAGAGGGTCTTCTCACCACA | |
| GGAACTCCTCTGGCTGGATGAGCTGATGCCAGAGCAGGAGAGAAGCATCCCTGAGAAGGG | |
| GCTGGAGCCAGAACACTCATTCAGTGGAAGTGACAGTGAAGATTCAGAGCTGATGTATCA | |
| GCCAAAGGCTCCAGAAATCAACATTTCTGTGAATTAGCTGGAGTAGGAGTCTGGGAGTGG | |
| AGACCC | |

In a search of public sequence databases, the NOV nucleic acid sequence, located on chromsome 17 has 2250 of 2788 bases (80%) identical to a gb:GENBANK-ID:AB038264|acc:AB038264.1 mRNA from Oryctolagus cuniculus (Oryctolagus cuniculus AE4b mRNA for anion exchanger 4b, complete cds). Public nucleotide databases include all GenBank databases and the GeneSeq patent database.

The disclosed NOV55 polypeptide (SEQ ID NO:128) encoded by SEQ ID NO:127 has 946 amino acid residues and is presented in Table 55B using the one-letter amino acid code. Signal P, Psort and/or Hydropathy results predict that NOV55 has no signal peptide and is likely to be localized extracellularly with a certainty of 0.8000.

TABLE 55B
Encoded NOV55 protein sequence.

| | |
|---|---|
| MEMKLPGQEGFEASSAPRNIPSGELDSNPDPGTGPSPDGPSDTESKELGVPKDPLLFIQL | (SEQ ID NO:128) |
| NELLGWPQALEWRETGRWVLFEEKLEVAAGRWSAPHVPTLALPSLQKLRSLLAEGLVLLD | |
| CPAQSLLELVEQVTRVESLSPELRGQLQALLLQRPQHYNQTTGTRPCWGESPSRKASDNE | |
| EAPLRDQCQNPLRQKLPPGAEAGTVLAGELGFLAQPLGAFVRLRNPVVLGSLTEVSLPSR | |
| FFCLLLGPCMLGKGYHEMGRAAAVLLSDPHSQQFQWSVRRASNLHDLLAALDAFLEEVTV | |
| LPPGRWDPTARIPPPKCLPSQHKRTSAEDRHRHGPHAHSPELQRTGRLFGGLIQDVRRKV | |
| PWYPSDFLDALHLQCFSAVLYIYLATVTNAITFGGLLGDATDGAQGVLESFLGTAVAGAA | |
| FCLMAGQPLTILSSTGPVLVFERLLFSFSRDYSLDYLPFRLWVGIWVATFCLVLVATEAS | |
| VLVRYFTRFTEEGFCALISLIFIYDAVGKMLNLTHTYPIQKPGSSAYGCLCQYPGPGGNE | |
| SQWIRTRPKDRDDIVSMDLGLINASLLPPPECTRQGGHPRGPGCHTVPDIAFFSLLLFLT | |
| SFFFAMALKCVKTSRFFPSVVRKGLSDFSSVLAILLGCGLDAFLGLATPKLMVPREFKPT | |
| LPGRGWLVSPFGANPWWWSVAAALPALLLSILIFMDQQITAVILNRMEYRLQKGAGFHLD | |
| LFCVAVLMLLTSALGLPWYVSATVISLAHMDSLRRESRACAPGERPNFLGIREQRLTGLV | |
| VFILTGASIFLAPVLKFIPMPVLYGIFLYMGVAALSSIQFTNRVKLLLMPAKHQPDLLLL | |
| RHVPLTRVHLFTAIQLACLGLLWIIKSTPAAIIFPLMLLGLVGVRKALERVFSPQELLWL | |
| DELMPEEERSIPEKGLEPEHSFSGSDSEDSELMYQPKAPEINISVN | |

A search of sequence databases reveals that the NOV55 amino acid sequence has 827 of 944 amino acid residues (87%) identical to, and 873 of 944 amino acid residues (92%) similar to, the 939 amino acid residue ptnr:TREMBLNEW-ACC:BAB18936 protein from Oryctolagus cuniculus (Rabbit) (ANION EXCHANGER 4B). Public amino acid databases include the GenBank databases, SwissProt, PDB and PIR.

NOV55 is expressed in at least kidney, testis. This information was derived by determining the tissue sources of the sequences that were included in the invention including but not limited to SeqCalling sources, Public EST sources, Literature sources, and/or RACE sources.

The disclosed NOV55 polypeptide has homology to the amino acid sequences shown in the BLASTP data listed in Table 55C.

TABLE 55C
BLAST results for NOV55

| Gene Index/ | | | Identity | Positives | |
|---|---|---|---|---|---|
| Identifier | Protein/Organism | Length (aa) | (%) | (%) | Expect |
| gi\|18560973\|ref\|XP_{—} | solute carrier | 959 | 938/962 | 940/962 | 0.0 |
| 038736.2\| | family 4, sodium | | (97%) | (97%) | |
| (XM_038736) | bicarbonate | | | | |
| | cotransporter, | | | | |
| | member 9 [Homo | | | | |
| | sapiens] | | | | |
| gi\|14582760\|gb\|AAK6 | anion exchanger | 959 | 937/962 | 939/962 | 0.0 |
| 9625.1\|AF332961_1 | AE4 [Homo sapiens | | (97%) | (97%) | |
| (AF332961) | | | | | |
| gi\|7363254\|dbj\|BAA9 | sodium | 957 | 936/960 | 938/960 | 0.0 |
| 3010.1\|(AB032762) | bicarbonate | | (97%) | (97%) | |
| | cotransporter 5 | | | | |
| | [Homo sapiens | | | | |
| gi\|13517508\|gb\|AAK2 | sodium | 990 | 938/986 | 940/986 | 0.0 |
| 8832.1\|AF313465_1 | bicarbonate | | (95%) | (95%) | |
| (AF313465) | cotransporter | | | | |
| | [Homo sapiens] | | | | |
| gi\|13249295\|gb\|AAK1 | anion exchanger | 945 | 922/962 | 926/962 | 0.0 |
| 6733.1\|AF336237_1 | AE4 [Homo | | (95%) | (95%) | |
| (AF336237) | sapiens] | | | | |

Table 55D lists the domain descriptions from DOMAIN analysis results against NOV55. This indicates that the NOV55 sequence has properties similar to those of other proteins known to contain this domain.

TABLE 55D
Domain Analysis of NOV55

gnl|Pfam|pfam00955, HCO3_cotransp, HCO3− transporter family. This
family contains Band 3 anion exchange proteins that exchange CL−/
HCO3−. This family also includes cotransporters of Na+/HCO3−.
CD-Length = 781 residues, 100.0% aligned
Score = 731 bits (1887), Expect = 0.0

The rabbit anion exchanger 4B protein is a member of the bicarbonate ion transporter Super family and is present on the apical membrance of beta-intercalated cells in the collecting ducts of the rabbit kidney (See Tsuganiezawa et al., J Biol Chem Dec. 1, 2000). The rabbit protein has sodium-independent anion exchanger activity when expressed in cultured COS-7 cells and Xenopus oocytes.

The acid-secreting alpha intercalated cells and bicarbonate-secreting beta intercalated cells are sites for modulation of urinary acid secretion, which in turn governs acid-base homeostasis. Mutations in the red cell anion exchanger gene, for instance, are correlated with familial distal renal tubular acidosis (Sec Bruce et al., Biochem Cell Biol 1998; 76(5): 723-728).

The disclosed NOV55 nucleic acid of the invention encoding a anion exchanger-like protein includes the nucleic acid whose sequence is provided in Table 55A or a fragment thereof. The invention also includes a mutant or variant nucleic acid any of whose bases may be changed from the corresponding base shown in Table 55A while still encoding a protein that maintains its anion exchanger-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 20 percent of the bases may be so changed.

The disclosed NOV55 protein of the invention includes the anion exchanger-like protein whose sequence is provided in Table 55B. The invention also includes a mutant or variant protein any of whose residues may be changed from the corresponding residue shown in Table 55B while still encoding a protein that maintains its anion exchanger-like activities and physiological functions, or a functional fragment thereof. In the mutant or variant protein, up to about 13 percent of the residues may be so changed.

The invention further encompasses antibodies and antibody fragments, such as F_{ab} or (F_{ab})₂, that bind immunospecifically to any of the proteins of the invention.

The above defined information for this invention suggests that this anion exchanger-like protein (NOV55) may function as a member of a “anion exchanger family”. Therefore, the NOV55 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV55 nucleic acids and proteins of the invention are useful in potential therapeutic applications implicated in diseases including but not limited to various pathologies and disorders as indicated below. For example, a cDNA encoding the anion exchanger-like protein (NOV55) may be useful in gene therapy, and the anion exchanger-like protein (NOV55) may be useful when administered to a subject in need thereof. By way of nonlimiting example, the compositions of the present invention will have efficacy for treatment of patients suffering from acidosis, alkalosis, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, cancer, tissue degeneration, bacterial/viral/parasitic infection, or other pathologies or conditions. The NOV55 nucleic acid encoding the anion exchanger-like protein of the invention, or fragments thereof, may further be useful in diagnostic applications, wherein the presence or amount of the nucleic acid or the protein are to be assessed.

NOV55 nucleic acids and polypeptides are further useful in the generation of antibodies that bind immuno-specifically to the novel NOV55 substances for use in therapeutic or diagnostic methods. These antibodies may be generated according to methods known in the art, using prediction from hydrophobicity charts, as described in the “Anti-NOVX Antibodies” section below. The disclosed NOV55 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV56

A disclosed NOV56 nucleic acid of 1083 nucleotides (also referred to as CG57348-01) encoding a PR_SET_domain protein-like protein is shown in Table 56A. The start and stop codons are in bold letters.

TABLE 56A
NOV56 nucleotide sequence.

(SEQ ID NO:129)

GGCGGCGAGGGTGCGGGGCGCGCTGCCATGGGCCTTCGCGGCGCCGCCGCGGCGCTGGTGGCGGCCGCAG
CAGCAGCAGCAGCGGCAGCGGCAGCGGTGGTGGCCGGGCCGCGGCGGCGGCGGCGAGGGTGCGGGGCGCG
CTGCCATGGGCCTGGCCGGGCTGCAGGCAAGAAGATGTCCAAGCCCCGCGCGCTGGAGGCGGCGGCGGCG
GCGGCAGCGACGGCCCCGGGCCTGGAGATGGTGGAGCGGAGGGGCCCGGGGAGGCCCCGCACCGATGGGG
AGAGCGTATTTACCGGGCAGTCAAAGATCTATTCCTACATGAGCCCGAACAAATGCTCTGGAATGCGTTT
CCCCCTTCAAGAAGAGAACTCGGTTACACATCACGAAGTCAAATGCCAGGGGAAACCATTAGCCGGAATC
TACAGGAAACGAGAAGAGAAAAGAAATACTGGGAACGCAGTACAGAGCGCCATGAAGTCCAAGAAACAGA
AGATCAAAGACGCCAGGAGAGGTCCCCTGCAAGGAAAAACACAACAGAATCACAAACTTACGGATTTCTA
CCCTGTCCGAAGGAGATCCAGGAAGAGCAAAGCCGACCTGCAGTCTGAAGAAAGGAAAAGAATAGATGAA
TTGATTGAAAGTGGGAAGGAAGAAGGAATGAAGATTGACCTCATCGATGGCAAAGGCAGGGGTGTGATTG
CCACCAAGCATTTCTCCCGGGGTGCCTTTGTGGTGGAATACCACGGGGACCTCATCGAGATCACCGACGC
CAAGAAACGGGAGGCTCTGTATGCACAGGACCCTTCCACGGGCTGCTACATGTACTATTTTCAGTATCTG
AGCAAAACCTACTGCGTGGATGCAACTAGAGAGACAAATCGCCCAGGAAGACCGATCAATCACAGCAAAT
GTGGGAACTGCCAAACCAAACTGCACGACATCGACGGCGTACCTCACCTCATCCTCATCGCCTCCCAAGA
CATCGCGGCTGGGGAGGAGCTCCTGTATGACTATGGGGACCGCAGCAAGGCTTCCATTGAAGCCCACCCG
TGGCTGAAGCATTAACCGGTGGGCCCCGCGCCC

In a search of public sequence databases, the NOV56 nucleic acid sequence, located on chromsome 12 has 1036 of 1086 bases (95%) identical to a gb:GENBANK-ID:AF287261|acc:AF287261.1 mRNA from Homo sapiens (Homo sapiens PR/SET domain containing protein 07 (SET07) mRNA, complete cds). Public nucleotide databases include all GenBank databases and the GeneSeq patent database.

The disclosed NOV56 polypeptide (SEQ ID NO:130) encoded by SEQ ID NO:129 has 345 amino acid residues and is presented in Table 56B using the one-letter amino acid code. Signal P, Psort and/or Hydropathy results predict that NOV56 has a signal peptide and is likely to be localized in the endoplasmic reticulum with a certainty of 0.600. The most likely cleavage site for a NOV56 peptide is between amino acids 22 and 23.

TABLE 56B
Encoded NOV56 protein sequence.

| | |
|---|---|
| MGLGGAAAALVAAAAAAAAAAAAVVAGPRRRRRGCGARCHGPGRAAGKKMSKPRALEAAA | (SEQ ID NO:130) |
| AAAATAPGLEMVERRGPGRPRTDGESVFTGQSKIYSYMSPNKCSGMRFPLQEENSVTHHE | |
| VKCQGKPLAGIYRKREEKRNTGNAVQSAMKSKKQKIKDARRGPLQGKTQQNHKLTDFYPV | |
| RRRSRKSKAELQSEERKRIDELIESGKEEGMKIDLIDGKGRGVIATKHFSRGAFVVEYHG | |
| DLIEITDAKKREALYAQDPSTGCYMYYFQYLSKTYCVDATRETNRPGRPINHSKCGNCQT | |
| KLHDIDGVPHLILIASQDIAAGEELLYDYGDRSKASIEAHPWLKH | |

A search of sequence databases reveals that the NOV56 amino acid sequence has 314 of 345 amino acid residues (91%) identical to, and 322 of 345 amino acid residues (93%) similar to, the 345 amino acid residue ptnr:SPTPEMBL-ACC:Q9NQR1 protein from Homo sapiens (Human) (PR/SET DOMAIN CONTAINING PROTEIN 07). Public amino acid databases include the GenBank databases, SwissProt, PDB and PIR.

NOV56 is expressed in at least Bone Marrow, Brain, Cervix, Dermis, Heart, Kidney, Liver, Lung, Lymph node, Pancreas, Parietal Lobe, Pituitary Gland, Placenta, Skin, Spinal Chord, Spleen, Thymus, Thyroid, Umbilical Vein, Adrenal Gland/Suprarenal gland, Aorta, Ascending Colon, Brain, Buccal mucosa, Cartilage, Cervix, Chorionic Villus, Colon, Coronary Artery, Duodenum, Heart, Kidney, Liver, Lung, Ovary, Parietal Lobe, Parotid Salivary glands, Peripheral Blood, Prostate, Retina, Salivary Glands, Small Intestine, Synovium/Synovial membrane, Testis, Tonsils, Umbilical Vein, and Urinary Bladder. Expression information was derived from the tissue sources of the sequences that were included in the derivation of the sequence of CG57348_{—}01. The sequence is predicted to be expressed in the following tissues because of the expression pattern of (GENBANK-ID: gb:GENBANK-ID:AF287261|acc:AF287261.1) a closely related Homo sapiens PR/SET domain containing protein 07 (SET07) mRNA, complete cds homolog in species Homo sapiens: Bone Marrow, Brain, Cervix, Dermis, Heart, Kidney, Liver, Lung, Lymph node, Pancreas, Parietal Lobe, Pituitary Gland, Skin, and Colon. This information was derived by determining the tissue sources of the sequences that were included in the invention including but not limited to SeqCalling sources, Public EST sources, Literature sources, and/or RACE sources.

The disclosed NOV56 polypeptide has homology to the amino acid sequences shown in the BLASTP data listed in Table 56C.

TABLE 56C
BLAST results for NOV56

| Gene Index/ | | Length | Identity | Positives | |
|---|---|---|---|---|---|
| Identifier | Protein/Organism | (aa) | (%) | (%) | Expect |
| gi\|15295688\|ref\|XP_{—} | similar to PR/SET | 403 | 306/359 | 306/359 | e−149 |
| 004668.4\| | domain containing | | (85%) | (85%) | |
| (XM_004668 | protein 07 (H. | | | | |
| | sapiens) [Homo | | | | |
| | sapiens] | | | | |
| gi\|15042945\|ref\|NP_{—} | PR/SET domain | 393 | 319/393 | 324/393 | e−139 |
| 065115.2\| | containing | | (81%) | (82%) | |
| (NM_020382) | protein 07 [Homo | | | | |
| | sapiens] | | | | |
| gi\|15303323\|ref\|XP_{—} | PR/SET domain | 240 | 145/165 | 150/165 | 3e−46 |
| 017300.2\| | containing | | (87%) | (90%) | |
| (XM_017300) | protein 07 [Homo | | | | |
| | sapiens] | | | | |
| gi\|7299871\|gb\|AAF55 | CG3307 gene | 689 | 100/230 | 143/230 | 2e−45 |
| 047.1\|(AE003704) | product | | (43%) | (61%) | |
| | [Drosophila | | | | |
| | melanogaster] | | | | |
| gi\|17554790\|ref\|NP_{—} | T26A5.7.p | 242 | 83/203 | 125/203 | 2e−33 |
| 498417.1\| | [Caenorhabditis | | (40%) | (60%) | |
| (NM_066016) | elegans] | | | | |

Table 56D lists the domain descriptions from DOMAIN analysis results against NOV56. This indicates that the NOV56 sequence has properties similar to those of other proteins known to contain this domain.

TABLE 56D
Domain Analysis of NOV56

gnl|Smart|smart00317, SET, SET (Su(var)3-9, Enhancer-of-zeste,
Trithorax) domain; Putative methyl transferase, based on outlier plant
homologues
CD-Length = 125 residues, 96.8% aligned
Score = 107 bits (266), Expect = 1e−24

Association of SET domain and myotubularin-related proteins modulates growth control. The PR domain of the Rb-binding zinc finger protein RIZ1 is a protein binding interface and is related to the SET domain functioning in chromatin-mediated gene expression.

SET domains appear to be protein-protein interaction domains. It has been demonstrated that SET domains mediate interactions with a family of proteins that display similarity with dual-specificity phosphatases (dsPTPases) [2]. A subset of SET domains have been called PR domains. These domains are divergent in sequence from other SET domains, but also appear to mediate protein-protein interaction [3].

The SET domain is a highly conserved, approximately 150-amino acid motif implicated in the modulation of chromatin structure. It was originally identified as part of a larger conserved region present in the Drosophila Trithorax protein and was subsequently identified in the Drosophila Su(var)₃-9 and ‘Enhancer of zeste’ proteins, from which the acronym SET is derived. Studies have suggested that the SET domain may be a signature of proteins that modulate transcriptionally active or repressed chromatin states through chromatin remodeling activities.

By sequencing cDNAs randomly selected from a cDNA library derived from a human immature myeloid cell line, Nomura et al. (1994) isolated a cDNA encoding SETDB1, which they called KIAA0067. The deduced SETDB1 protein has 1,291 amino acids. Northern blot analysis detected SETDB1 expression in all 16 human tissues examined.

In the course of searching sequence databases for proteins containing SET domains, Harte et al. (1999) identified the SETDB1 sequence. They determined that SETDB1 has a C-terminal SET domain that is well-conserved except that it contains a 347-amino acid insertion between its most highly conserved regions. The authors found that the C. elegans YNCA gene product is highly similar to SETDB1 and also contains a bifurcated SET domain.

Nomura et al. (1994) mapped the SETDB1 gene to chromosome 1 using a somatic cell hybrid mapping panel. By FISH and radiation hybrid mapping, Harte et al. (1999) mapped the SETDB1 gene to 1q21.

The disclosed NOV56 nucleic acid of the invention encoding a PR_SET_domain protein-like protein includes the nucleic acid whose sequence is provided in Table 56A or a fragment thereof. The invention also includes a mutant or variant nucleic acid any of whose bases may be changed from the corresponding base shown in Table 56A while still encoding a protein that maintains its PR_SET_domain protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV56 protein of the invention includes the PR_SET_domain protein-like protein whose sequence is provided in Table 56B. The invention also includes a mutant or variant protein any of whose residues may be changed from the corresponding residue shown in Table 56B while still encoding a protein that maintains its PR_SET_domain protein-like activities and physiological functions, or a functional fragment thereof. In the mutant or variant protein, up to about 9 percent of the residues may be so changed. The invention further encompasses antibodies and antibody fragments, such as F_{ab} or (F_{ab})₂, that bind immunospecifically to any of the proteins of the invention.

The above defined information for this invention suggests that this PR_SET_domain protein-like protein (NOV56) may function as a member of a “PR_SET_domain protein family”. Therefore, the NOV56 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV56 nucleic acids and proteins of the invention are useful in potential therapeutic applications implicated in diseases including but not limited to various pathologies and disorders as indicated below. For example, a cDNA encoding the PR_SET_domain protein-like protein (NOV56) may be useful in gene therapy, and the PR_SET_domain protein-like protein (NOV56) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV56 nucleic acid encoding the PR_SET_domain protein-like protein of the invention, or fragments thereof, may further be useful in diagnostic applications, wherein the presence or amount of the nucleic acid or the protein are to be assessed.

NOV56 nucleic acids and polypeptides are further useful in the generation of antibodies that bind immuno-specifically to the novel NOV56 substances for use in therapeutic or diagnostic methods. These antibodies may be generated according to methods known in the art, using prediction from hydrophobicity charts, as described in the “Anti-NOVX Antibodies” section below. The disclosed NOV56 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV57

A disclosed NOV57 nucleic acid of 5896 nucleotides (also referred to as CG57650-01) encoding a non-muscle myosin heavy chain B-like protein is shown in Table 57A. The start and stop codons are in bold letters.

TABLE 57A
NOV57 nucleotide sequence.

| | |
|---|---|
| GGCGGTGCTGCGGGACGAAGGCGAGGAGGAGGCGGAGGTGGAGCTGGCGGAGAGCGGGAG | (SEQ ID NO:131) |
| GCGGCTGCGACTGCCGCGGGACCAGATCCAGCGCATGAACCCGCGCCAAGTTCAGCAAGG | |
| CCGAGGACATGGCCGAGCTGACCTGCCTCAACGAGGCCTCGGTCCTGCACAACCTCCGGG | |
| AGCGGTACTACTCCGGCCTCATCTACACGTACTCCGGCCTTTTCTGTGTGGTCATCAACC | |
| CGTACAAGCAGCTTCCCATCTACACAGAAGCCATTGTGGAGATGTACCGGGGCAAGAAGC | |
| GCCACGAGGTGCCACCCCACGTGTACGCAGTGACCGAGGGGGCCTATCGGAGCATGCTGC | |
| AGGATCGTGAGGACCAGTCCATTCTCTGCACGGGAGAGTCTGGAGCTGGGAAGACGGAAA | |
| ACACCAAGAAGGTCATCCAGTACCTCGCCCACGTGGCGTCGTCTCCAAAGGGCAGGAAGG | |
| AGCCGGGTGTCCCCGGTGAGCTGGAGCGGCAGCTGCTTCAGGCCAACCCCATCCTAGAGG | |
| CCTTTGGCAATGCCAAGACAGTGAAGAATGACAACTCCTCCCGATTCGGCAAATTCATCC | |
| GCATCAACTTTGATGTTGCCGGGTACATCGTGGGCGCCAACATTGAGACCTGTCTGCTGG | |
| AGAAGTCGCGGGCCATCCGCCAGGCCAAGGACGAGTGCAGCTTCCACATCTTCTACCAGC | |
| TGCTGGGGGGCGCTGGAGAGCATGGCTGCCGAGAACTCCTCCTCGAGCCCTGCTCCCACT | |
| ACCGGTTCCTGACCAACGGGCCGTCATCCTCTCCCGGCCAGGAGCGGGAACTCTTCCAGG | |
| AGACGCTGGAGTCGCTGCGGGTCCTGGGATTCAGCCACGAGGAAATCATCTCCATGCTGC | |
| GGATGGTCTCAGCAGTTCTCCAGTTTGGCAACATTGCCTTGAAGAGAGAACGGAACACCG | |
| ATCAAGCCACCATGCCTGACAACACAGCTGCACAGAAGCTCTGCCGCCTCTTGGGACTGG | |
| GGGTGACGGATTTCTCCCGAGCCTTGCTCACCCCTCGCATCAAAGTTGGCCGAGACTATG | |
| TGCAGAAAGCCCAGACTAAGGAACAGGCTGACTTCGCGCTGGAGGCCCTGGCCAAGGCCA | |
| CCTACGAGCGCCTCTTCCGCTGGCTGGTTCTGCGCCTCAACCGGGCCTTGGACCGCAGCC | |
| CCCGCCAAGGCGCCTCCTTCCTGGGCATCCTGGACATCGCGGGCTTTGAGATCTTCCAGC | |
| TGAACTCCTTCGAGCAGCTCTGCATCAACTACACCAACGAGAAGCTGCAGCAGCTCTTCA | |
| ACCACACCATGTTCGTGCTGGAGCAGGAGGAGTACCAGCGTGAGGGCATCCCCTGGACCT | |
| TGGAACAGGTGAGCAGCCTGGGCGACGGCCCACCAGGTGGCCGCCCCCGTCGGGGTATGT | |
| TCCGGACAGTGGGACAGCTCTACAAGGAGTCCCTGAGCCGCCTCATGGCCACACTCAGCA | |
| ACACCAACCCCAGTTTTGTCCGCTGCATTGTCCCCAACCACGAGAAGAGGGTCGGGAAGC | |
| TGGAGCCGCGGCTGGTGCTGGACCAGCTTCGCTGCAACGGGGTCCTGGAGGGCATCCGCA | |
| TCTGTCGCCAGGGCTTCCCCAACCGCATCCTCTTCCAGGAGTTCCGGCAGCGATACGAGA | |
| TCCTGACACCCAATGCCATCCCCAAGGGCTTCATGGATGGGAAGCAGGCCTGTGAAAAGA | |
| TGATCCAGGCGCTGGAACTGGACCCCAACCTCTACCGCGTGGGACAGAGCAAGATCTTCT | |
| TCCGGGCTGGGGTCCTGGCCCAGCTGGAAGAGGAGCGAGACCTGAAGGTCACCGACATCA | |
| TCGTCTCCTTCCAGGCAGCTGCCCGGGGATACCTGGCTCGCAGGGCCTTCCAGAAGCGCC | |
| AGCAGCAGCAGAGCGCCCTGAGGGTGATGCAGCGGAACTGCGCGGCCTACCTCAAGCTGA | |
| GACACTGGCAGTGGTGGCGGCTGTTTACCAAGGTGAAGCCACTGCTGCAGGTGACGCGGC | |
| AGGATGAGGTGCTGCAGGCACGGGCCCAGGAGCTGCAGAAAGTGCAGGAGCTACAGCAGC | |
| AGAGCGCCCGCGAAGTTGGGGAGCTCCAGGGCCGAGTGACACAGCTGGAAGAGGAGCGCG | |
| CCCGCCTGGCAGAGCAATTGCGAGCAGAGGCAGAACTGTGTGCAGAGGCCGAGGAGACGC | |
| GGGGGAGGCTGGCAGCCCGCAAGCAGGAGCTGGAGCTGGTGGTGTCAGAGCTGGAGGCTC | |
| GCGTGGGCGAGGAGGAGGAGTGCAGCCGTCAAATGCAAACCGAGAAGAAGAGGCTACAGC | |
| AGCACATACAGGAGCTAGAGGCCCACCTTGAGGCTGAGGAGGGTGCGCGGCAGAAGCTGC | |
| AGCTGGAGAAGGTGACGACAGAGGCAAAAATGAAGAAATTTGAAGAGGACCTGCTGCTCC | |
| TGGAAGACCAGAATTCCAAGCTGAGCAAGGAACTGCTGGAAGATCGTCTGGCCGAGTTCT | |
| CATCCCAGGCAGCTGAGGAGGAGGAGAAGGTCAAGAGCCTCAATAAGCTACGGCTCAAAT | |
| ATGAGGCCACAATCGCAGACATGGAGGACCGCCTACGGAAGGAGGAGAAGGGTCGCCAGG | |
| AGCTGGAGAAGCTGAAGCGGAGGCTGGATGGGGAGAGCTCAGAGCTGCAGGAGCAGATGG | |
| TGGAGCAGCAACAGCGGGCAGAGGAGCTGCGGGCCCAGCTGGGCCGGAAGGAGGAGGAGC | |
| TGCAGGCTGCCCTGGCCAGGGCAGAAGACGAGGGTGGGGCCCGGGCCCAGCTGCTGAAAT | |
| CCCTGCGGGAGGCTCAAGCAGCCCTGGCCGAGGCCGAGGCCCAGGAGGACCTGGAGTCTG | |
| AGCGTGTGGCCAGGACCAAGGCGGAGAAGCAGCGCCGGGACCTGGGCGAGGAGCTGGAGG | |
| CGCTGCGGGGCGAGCTGGAGGACACGCTGGACTCCACCAACGCACAGCAGGAGCTCAGGT | |
| CCAAGAGGGAACAGGAGGTGACGGAGCTGAAGAAGACTCTGGAGGAGGAGACTCGCATCC | |
| ACGAGGCGGCAGTGCAGGAGCTGAGGCAGCGCCACGGCCAGGCCCTGGGGGAGCTGGCGG | |
| AGCAGCGGAGGCGCCGCCTGGAGTTACAGCTGCAGGAGGTGCAGGGCCGGGCTGGTGATG | |
| GGGAGAGGGCACGAGCGGAGGCTGCTGAGAAGGTCCCTTCCCTGCAGGCTGAACTGGAGA | |
| ATGTGTCTGGGGCGCTGAACGAGGCTGAGTCCAAAACCATCCGTCTTAGCAAGGAGCTGA | |
| GCAGCACAGAAGCCCAGCTGCACGATGCCCAGGAGCTGCTGCAGGAGGAGACCAGGGCGA | |
| AATTGGCCTTGGGGTCCCGGGTGCGAGCCATGGAGGCTGAGGCAGCCGGGCTGCGTGAGC | |
| AGCTGGAGGAGGAGGCAGCTGCCAGGGAACGGGCGGACCACCAACCACCCTCTCTCTCCT | |
| CCCCTCAGCTTTCCGAGTGGCGGCGGCGCCAGGAGGAGGAGGCAGGGGCACTGGAGGCAG | |
| GGGAGGAGGCACGGCGCCGGGCAGCCCGGGAGGCCGAGGCCCTGACCCAGCGCCTGGCAG | |
| AAAAGACAGAGACCGTGGATCGGCTGGAGCGGGGCCGCCGCCGGCTGGGGCAGGAGCTGG | |
| ACGACGCCACCATGGACCTGGAGCAGCAGCGGCAGCTTGTGAGCACCCTGGAGAAGAAGC | |
| AGCGCAAGTTTGACCAGCTTCTGGCAGAGGAGAAGGCAGCTGTACTTCGGGCAGTGGAGG | |
| AACGTGAGCGGGCCGAGGCAGAGGGCCGGGAGCGTGAGGCTCGGGCCCTGTCACTGACAC | |
| GGGCACTGGAGGAGGAGCAGGAGGCACGTGAGGAGCTGGAGCGGCAGAACCGGGCCCTGC | |
| GGGCTGAGCTGGAGGCACTGCTGAGCAGCAAGGATGACGTCGGCAAGAGCGTGCATGAGC | |
| TGGAACGAGCCTGCCGGGTAGCAGAACAGGCAGCCAATGATCTGCGAGCACAGGTGACAG | |
| AACGTGAGCGGGCCGAGGCAGAGGGCCGGGAGCGTGAGGCTCGGGCCCTGTCACTGACAC | |
| AGGCTCTCAAGACTCAGCATGAGCGTGACCTGCAGGGCCGTGATGAGGCTGGTGAAGAGA | |
| GGCGGAGGCAGCTGGCCAAGCAGCTGAGAGATGCAGAGGTGGAGCGGGATGAGGAGCGGA | |
| AGCAGCGCACTCTGGCCGTGGCTGCCCGCAAGAAGCTGGAGGGAGAGCTGGAGGAGCTGA | |
| AGGCTCAGATGGCCTCTGCCGGCCAGGGCAAGGAGGAGGCGGTGAAGCAGCTTCGCAAGA | |
| TGCAGGCCCAGATGAAGGAGCTATGGCGGGAGGTGGAGGAGACACGCACCTCCCGGGAGG | |
| AGATCTTCTCCCAGAATCGGGAAAGTGAAAAGCGCCTCAAGGGCCTGGAGGCTGAGGTGC | |
| TGCGGCTGCAGGAGGAACTGGCCGCCTCGGACCGTGCTCGGCGGCAGGCCCAGCAGGACC | |
| GGGATGAGATGGCAGATGAGGTGGCCAATGGTAACCTTAGCAAGGCAGCCATTCTGGAGG | |
| AGAAGCGTCAGCTGGAGGGGCGCCTGGGGCAGTTGGAGGAAGAGCTGGAGGAGGAGCAGA | |
| CAACTCAGAGCTGCTCAATGACCGCTACCGCAAGCTGCTCCTGCAGGTAGAGTCACTGAC | |
| CACAGAGCTGTCAGCTGAGCGCAGTTTCTCAGCCAAGGCAGAGAGCGGGCGGCAGCAGCT | |
| GGAACGGCAGATCCAGGAGCTACGGGGACGCCTGGGTGAGGAGGATGCTGGGGCCCGTGC | |
| CCGCCACAAGATGACCATTGCTGCCCTTGAGTCTAAGTTGGCCCAGGCTGAGGAGCAGCT | |
| AGAGCAAGAGACCAGAGAGCGCATCCTCTCTGGAAAGCTGGTGCCCAAAAGTTAAGAAGC | |
| GGCTTAAAGAGGTGGTGCTCCAGGTGGAGGAGGAGCGGAGGGTGGCTGACCAGCTCCGGG | |
| ACCAGCTGGAGAAGGGAAACCTTCGAGTCAAGCAGCTGAAGCGGCAGCTGGAGGAGGCCG | |
| AGGAGGAGGCATCCCGGGCTCAGGCCGGCCGCCGGAGGCTGCAGCGTGAGCTGGAAGATG | |
| TCACAGAGTCGGCCGAGTCCATGAACCGTGAAGTGACCACACTGAGGAACCGGCTTCGAC | |
| GCGGCCCCCTCACCTTCACCANCCGCACGGTGCGCCAGGTCTTCCGACTAGAGGAGGGCG | |
| TGGCATCCGACGAGGAGGCAGAGGAAGCACAGCCTGGGTCTGGGCCATCCCCTCTCACTC | |
| CTGCTGCTGCCCATGCTCTGCCCTCCCTTCTGGTTGCTCTGAGGGTTCGGAGCTTCCCTC | |
| TGGGACTAAAGGAGTGTCCTTTACCCTCCCAGCCTCCCGGCTCTGGCAGAAATAAACTCC | |
| AACCCGAATGGAAAAA | |

In a search of public sequence databases, the NOV57 nucleic acid sequence, located on chromsome 10 has 3509 of 4934 bases (71%) identical to a gb:GENBANK-ID:RABMHCP|acc:M77812.1 mRNA from Oryctolagus cuniculus (Rabbit myosin heavy chain mRNA, complete cds). Public nucleotide databases include all GenBank databases and the GeneSeq patent database.

The disclosed NOV57 polypeptide (SEQ ID NO:132) encoded by SEQ ID NO:131 has 1673 amino acid residues and is presented in Table 57B using the one-letter amino acid code. Signal P, Psort and/or Hydropathy results predict that NOV57 has no signal peptide and is likely to be localized in the nucleus with a certainty of 0.9600.

TABLE 57B
Encoded NOV57 protein sequence.

| | |
|---|---|
| MAELTCLNEASVLHNLRERYYSGLIYTYSGLFCVVINPYKQLPIYTEAIVEMYRGKKRHE | (SEQ ID NO:132) |
| VPPHVYAVTEGAYRSMLQDREDQSILCTGESGAGKTENTKKVIQYLAHVASSPKGRKEPG | |
| VPGELERQLLQANPILEAFGNAKTVKNDNSSRFGKFIRINFDVAGYIVGANIETCLLEKS | |
| RAIRQAKDECSFHIFYQLLGGAGEHGCRELLLEPCSHYRFLTNGPSSSPGQERELFQETL | |
| ESLRVLGFSHEEIISMLRMVSAVLQFGNIALKRERNTDQATMPDNTAAQKLCRLLGLGVT | |
| DFSRALLTPRIKVGRDYVQKAQTKEQADFALEALAKATYERLFRWLVLRLNRALDRSPRQ | |
| GASFLGILDIAGFEIFQLNSFEQLCINYTNEKLQQLFNHTMFVLEQEEYQREGIPWTFLD | |
| FGLDLQPCIDLIERPANPPGLLALLDEEDWFPKATDKSFVEKVAQEQGGHPKFQRPRHLR | |
| DQADFSVLHYAGKVDYKANEWLMKNMDPLNDNVAALLHQSTDRLTAEIWKDVEGIVGLEQ | |
| VSSLGDGPPGGRPRRGMFRTVGQLYKESLSRLMATLSNTNPSFVRCIVPNHEKRVGKLEP | |
| RLVLDQLRCNGVLEGIRICRQGFPNRILFQEFRQRYEILTPNAIPKGFMDGKQACEKMIQ | |
| ALELDPNLYRVGQSKIFFRAGVLAQLEEERDLKVTDIIVSFQAAARGYLARRAFQKRQQQ | |
| QSALRVMQRNCAAYLKLRHWQWWRLFTKVKPLLQVTRQDEVLQARAQELQKVQELQQQSA | |
| REVGELQGRVTQLEEERARLAEQLRAEAELCAEAEETRGRLAARKQELELVVSELEARVG | |
| EEEECSRQMQTEKKRLQQHIQELEAHLEAEEGARQKLQLEKVTTEAKMKKFEEDLLLLED | |
| QNSKLSKELLEDRLAEFSSQAAEEEEKVKSLNKLRLKYEATIADMEDRLRKEEKGRQELE | |
| KLKRRLDGESSELQEQMVEQQQRAEELRAQLGRKEEELQAALARAEDEGGARAQLLKSLR | |
| EAQAALAEAEAQEDLESERVARTKAEKQRRDLGEELEALRGELEDTLDSTNAQQELRSKR | |
| EQEVTELKKTLEEETRIHEAAVQELRQRHGQALGELAEQLEQARRKGAWEKTRLALEAEV | |
| SELRAILSSLQTARQEGEQRRRRLELQLQEVQGRAGDGERARAEAAEKVPSLQAELENVS | |
| GALNEAESKTIRLSKELSSTEAQLHDAQELLQEETRAKLALGSRVRAMEAEAAGLREQLE | |
| EEAAARERADHQPPSLSSPQLSEWRRRQEEEAGALEAGEEARRRAAREAEALTQRLAEKT | |
| ETVDRLERGRRRLGQELDDATMDLEQQRQLVSTLEKKQRKFDQLLAEEKAAVLRAVEERE | |
| RAEAEGREREARALSLTRALEEEQEAREELERQNRALRAELEALLSSKDDVGKSVHELER | |
| ACRVAEQAANDLRAQVTELEDELTAAEDAKLRLEVTVQALKTQHERDLQGRDEAGEERRR | |
| QLAKQLRDAEVERDEERKQRTLAVAARKKLEGELEELKAQMASAGQGKEEAVKQLRKMQA | |
| QMKELWREVEETRTSREEIFSQNRESEKRLKGLEAEVLRLQEELAASDRARRQAQQDRDE | |
| MADEVANGNLSKAAILEEKRQLEGRLGQLEEELEEEQTTQSCSMTATASCSCR | |

A search of sequence databases reveals that the NOV57 amino acid sequence has 1149 of 1661 amino acid residues (69%) identical to, and 1395 of 1661 amino acid residues (83%) similar to, the 1976 amino acid residue ptnr:SWISSNEW-ACC:P35580 protein from Homo sapiens (Human) (MYOSIN HEAVY CHAIN, NONMUSCLE TYPE B (CELLULAR MYOSIN HEAVY CHAIN, TYPE B) (NMMHC-B))(. Public amino acid databases include the GenBank databases, SwissProt, PDB and PIR.

NOV57 is expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, Public EST sources, Literature sources, and/or RACE sources.

The disclosed NOV57 polypeptide has homology to the amino acid sequences shown in the BLASTP data listed in Table 57C.

TABLE 57C
BLAST results for NOV57

| Gene Index/ | | Length | Identity | Positives | |
|---|---|---|---|---|---|
| Identifier | Protein/Organism | (aa) | (%) | (%) | Expect |
| gi\|13928704\|ref\|NP_{—} | myosin heavy | 1976 | 1144/1664 | 1389/1664 | 0.0 |
| 113708.1\| | chain 11 [Rattus | | (68%) | (82%) | |
| (NM_031520) | norvegicus] | | | | |
| gi\|212449\|gb\|AAA489 | nonmuscle myosin | 1976 | 1148/1664 | 1395/1664 | 0.0 |
| 85.1\|(M93676) | heavy chain | | (68%) | (82%) | |
| | [Gallus gallus] | | | | |
| gi\|1346640\|sp\|P3558 | Myosin heavy | 1976 | 1149/1664 | 1395/1664 | 0.0 |
| 0\|MYHA_HUMAN | chain, nonmuscle | | (69%) | (83%) | |
| | type B (Cellular | | | | |
| | myosin heavy | | | | |
| | chain, type B) | | | | |
| | (Nonmuscle myosin | | | | |
| | heavy chain-B) | | | | |
| | (NMMHC-B) | | | | |
| gi\|212451\|gb\|AAA489 | nonmuscle myosin | 1997 | 1148/1685 | 1395/1685 | 0.0 |
| 87.1\|(M93676) | heavy chain | | (68%) | (82%) | |
| | [Gallus gallus | | | | |
| gi\|212450\|gb\|AAA489 | nonmuscle myosin | 1986 | 1148/1674 | 1395/1674 | 0.0 |
| 86.1\|(M93676) | heavy chain | | (68%) | (82%) | |
| | [Gallus gallus] | | | | |

Tables 57D-H list the domain descriptions from DOMAIN analysis results against NOV57. This indicates that the NOV57 sequence has properties similar to those of other proteins known to contain this domain.

TABLE 57D
Domain Analysis of NOV57

gnl|Pfam|pfam00063, myosin_head, Myosin head (motor domain).
CD-Length = 670 residues, 99.6% aligned
Score = 922 bits (2384), Expect = 0.0

TABLE 57E
Domain Analysis of NOV57

gnl|Smart|smart00242, MYSc, Myosin. Large ATPases.; ATPase;
molecular motor. Muscle contraction consists of a cyclical interaction
between myosin and actin. The core of the myosin structure is similar
in fold to that of kinesin.
CD-Length = 688 residues, 98.4% aligned
Score = 866 bits (2238), Expect = 0.0

TABLE 57F
Domain Analysis of NOV57

gnl|Pfam|pfam01576, Myosin_tail, Myosin tail. The myosin molecule is a
multi-subunit complex made up of two heavy chains and four light
chains it is a fundamental contractile protein found in all eukaryote
cell types. This family consists of the coiled-coil myosin heavy chain
tail region. The coiled-coil is composed of the tail from two
molecules of myosin. These can then assemble into the macromolecular
thick filament. The coiled-coil region provides the structural
backbone the thick filament.
CD-Length = 860 residues, 78.4% aligned
Score = 344 bits (882), Expect = 3e−95

TABLE 57G
Domain Analysis of NOV57

gnl|Pfam|pfam01496, V_ATPase_sub_a, V-type ATPase 116 kDa
subunit family. This family consists of the 116 kDa V-type ATPase
(vacuolar (H+)-ATPases) subunits, as well as V-type ATP synthase
subunit i. The V-type ATPases family are proton pumps that acidify
intracellular compartments in eukaryotic cells for example yeast
central vacuoles, clathrin-coated and synaptic vesicles. They have
important roles in membrane trafficking processes. The 116 kDa
subunit (subunit a) in the V-type ATPase is part of the V0 func-
tional domain responsible for proton transport. The a subunit is a
transmembrane glycoprotein with multiple putative transmembrane
helices it has a hydrophilic amino terminal and a hydrophobic
carboxy terminal. It has roles in proton transport and assembly of
the V-type ATPase complex. This subunit is encoded by two homologous
gene in yeast VPH1 and STV1.
CD-Length = 703 residues, 34.3% aligned
Score = 57.0 bits (136), Expect = 8e−09

TABLE 57H
Domain Analysis of NOV57

gnl|Pfam|pfam00769, ERM, Ezrin/radixin/moesin family. This family
of proteins contain a band 4.1 domain (pfam00373), at their amino
terminus. This family represents the rest of these proteins.
CD-Length = 365 residues, 43.6% aligned
Score = 52.0 bits (123), Expect = 3e−07

Myosins constitute a large superfamily of actin-dependent molecular motors. Phylogenetic analysis currently places myosins into 15 classes. The conventional myosins which form filaments in muscle and non-muscle cells form class II. There has been extensive characterization of these myosins and much is known about their function. With the exception of class I and class V myosins, little is known about the structure, enzymatic properties, intracellular localization and physiology of most unconventional myosin classes. (See Sellers JR, 2000, Biochim Biophys Acta 1496:3-22). The discovery in of a huge diversity within the myosin superfamily has been coupled with an understanding of the role of these motor proteins in various cellular functions. Extensive studies have revealed that myosin isoforms are not only involved in muscle contraction but also in crucial functions of many specialized mammalian cells such as melanocytes, kidney and intestinal brush border microvilli, nerve growth cones or inner ear hair cells. A search for genes involved in the pathology of human genetic deafness resulted in identification of three novel myosins: myosin VI, myosin VIIA and, very recently, myosin XV. Recently, mutations have been detected within these genes that have been found to affect the hearing process (See Redowicz M J, 1999, J Muscle Res Cell Motil 20:241-8). Class II non-muscle myosins are implicated in diverse biological processes such as cytokinesis, cellularization, cell shape changes and gastrulation. Two distinct non-muscle myosin heavy chain genes have been reported in all vertebrates: non-muscle myosin heavy chain-A (NMHC-A) and -B (NMHC-B). Whole mount in situ hybridization with tailbud stage embryos of Xenopus showed that NMHC-A mRNA is predominantly expressed in the epidermis, whereas NMHC-B mRNA is expressed in the somites, brain, eyes and branchial arches. Interestingly, the expression of NMHC-B in developing somites was gradually restricted to the center of each somite as differentiation proceeds. DAPI nuclear staining demonstrated that NMHC-B mRNA is colocalized with the nuclei or perinuclear area. In animal cap experiments, treatment with activin A or ectopic expression of Xbra and an activated form of Xlim1 markedly up-regulates NMHC-B as well as muscle actin mRNAs and slightly down-regulates NMHC-A mRNA, consistent with NMHC-B expression in the somitic muscle and NMHC-A expression in the epidermis.(See Bhatia et al., 1998, Mech Dev 78:33-6).

The disclosed NOV57 nucleic acid of the invention encoding a non-muscle myosin heavy chain B-like protein includes the nucleic acid whose sequence is provided in Table 57A or a fragment thereof. The invention also includes a mutant or variant nucleic acid any of whose bases may be changed from the corresponding base shown in Table 57A while still encoding a protein that maintains its non-muscle myosin heavy chain B-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 29 percent of the bases may be so changed.

The disclosed NOV57 protein of the invention includes the non-muscle myosin heavy chain B-like protein whose sequence is provided in Table 57B. The invention also includes a mutant or variant protein any of whose residues may be changed from the corresponding residue shown in Table 57B while still encoding a protein that maintains its non-muscle myosin heavy chain B-like activities and physiological functions, or a functional fragment thereof. In the mutant or variant protein, up to about 31 percent of the residues may be so changed.

The invention further encompasses antibodies and antibody fragments, such as F_{ab} or (F_{ab})₂ that bind immunospecifically to any of the proteins of the invention.

The above defined information for this invention suggests that this non-muscle myosin heavy chain B-like protein (NOV57) may function as a member of a “non-muscle myosin heavy chain B family”. Therefore, the NOV57 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV57 nucleic acids and proteins of the invention are useful in potential therapeutic applications implicated in diseases including but not limited to various pathologies and disorders as indicated below. For example, a cDNA encoding the non-muscle myosin heavy chain B-like protein (NOV57) may be useful in gene therapy, and the non-muscle myosin heavy chain B-like protein (NOV57) may be useful when administered to a subject in need thereof. By way of nonlimiting example, the compositions of the present invention will have efficacy for treatment of patients suffering from hypertension and vasospasm of the coronary and cerebral arteries, coronary artery spasm, artherosclerosis, hypertrophic cardiomyopathy, inflammatory diseases such as asthma, cancer, or other pathologies or conditions. The NOV57 nucleic acid encoding the non-muscle myosin heavy chain B-like protein of the invention, or fragments thereof, may further be useful in diagnostic applications, wherein the presence or amount of the nucleic acid or the protein are to be assessed.

NOV57 nucleic acids and polypeptides are further useful in the generation of antibodies that bind immuno-specifically to the novel NOV57 substances for use in therapeutic or diagnostic methods. These antibodies may be generated according to methods known in the art, using prediction from hydrophobicity charts, as described in the “Anti-NOVX Antibodies” section below. The disclosed NOV57 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV58

A disclosed NOV58 nucleic acid of 688 nucleotides (also referred to as CG57766-01) encoding a plasma retinol binding protein-like protein is shown in Table 58A. The start and stop codons are in bold letters.

TABLE 58A
NOV58 nuclcotidc sequence.

(SEQ ID NO:133)

CCCAACCACGGCCAGGCTTGCGCGCGGTTCCCCTCCCGGTGGGCGGATTCCTGGGCAAGATGAAGTGGT
GTGGGCGCTCTTGCTGTTGGCGGCGCTGGGCAGCGGCCGCGCGGAGCGCGACTGCCGAGTGAGCAGCTTC
CGAGTCAAGGAGAACTTCGACAAGGCTCGCTTCTCTGGGACCTGGTACGCCATGGCCAAGAAGGACCCCG
AGGGCCTCTTTCTGCAGGACAACATCGTCGCGGAGTTCTCCGTGGACGAGACCGGCCAGATGAGCGCCAC
AGCCAAGGGCCGAGTCCGTCTTTTGAATAACTGGGACGTGTGCGCAGACATGGTGGGCACCTTCACAGAC
ACCGAGGACCCTGCCAAGTTCAAGATGAAGTACTGGGGCGTAGCCTCCTTTCTCCAGAAAGGAAATGATG
TGGCACCTGTGCTGACAGCTACTCCTTCGTGTTTTCCCGGGACCCCAACGGCCTGCCCCCAGAAGCGCAG
AAGATTGTAAGGCAGCGGCAGGAGGAGCTGTGCCTGGCCAGGCAGTACAGGCTGATCGTCCACAACGGTT
ACTGCGATGGCAGATCAGAAAGAAACCTTTTGTAGCAAGGGCGAATTCCAGCACACTG

In a search of public sequence databases, the NOV58 nucleic acid sequence, located on chromsome 10q23-24 has 657 of 673 bases (97%) identical to a gb:GENBANK-ID:HSRBP1|acc:X00129.1 mRNA from Homo sapiens (Human mRNA for retinol binding protein (RBP)). Public nucleotide databases include all GenBank databases and the GeneSeq patent database.

The disclosed NOV58 polypeptide (SEQ ID NO:134) encoded by SEQ ID NO:133 has 201 amino acid residues and is presented in Table 58B using the one-letter amino acid code. Signal P, Psort and/or Hydropathy results predict that NOV58 has a signal peptide and is likely to be localized extracellularly with a certainty of 0.3700. The most likely cleavage site for a NOV58 peptide is between amino acids 18 and 19.

TABLE 58B
Encoded NOV58 protein sequence.

| | |
|---|---|
| MKWVWALLLLAALGSGRAERDCRVSSFRVKENFDKARFSGTWYAMAKKDPEDLFLQDNIV | (SEQ ID NO:134) |
| AEFSVDETGQMSATAKGRVRLLNNWDVCADMVGTFTDTEDPAKFKMKYWGVASFLQKGND | |
| DHWIVDTDYDTYAVQYSCRLLNLDGTCADSYSFVFSRDPNGLPPEAQKIVRQRQEELCLA | |
| RQYRLIVHNGYCDGRSERNLL | |

A search of sequence databases reveals that the NOV58 amino acid sequence has 196 of 201 amino acid residues (97%) identical to, and 197 of 201 amino acid residues (98%) similar to, the 199 amino acid residue ptnr:SWISSNEW-ACC:P02753 protein from Homo sapiens (Human) (PLASMA RETINOL-BINDING PROTEIN PRECURSOR(PRBP) (RBP)). Public amino acid databases include the GenBank databases, SwissProt, PDB and PIR.

NOV58 is expressed in at least adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, Public EST sources, Literature sources, and/or RACE sources.

The disclosed NOV58 polypeptide has homology to the amino acid sequences shown in the BLASTP data listed in Table 58C.

TABLE 58C
BLAST results for NOV58

| | | | | Po | |
|---|---|---|---|---|---|
| Gene Index/ | | Length | Identity | sitives | |
| Identifier | Protein/Organism | (aa) | (%) | (%) | Expect |
| gi\|18088326\|gb\|AAH2 | Similar to | 201 | 200/201 | 200/201 | e−113 |
| 0633.1\|AAH20633 | retinol binding | | (99%) | (99%) | |
| (BC020633 | protein 4, plasma | | | | |
| | [Homo sapiens] | | | | |
| gi\|2136468\|pir\|\|I46 | retinol binding | 201 | 186/201 | 195/201 | e−107 |
| 257 | protein precursor - | | (92%) | (96%) | |
| | horse | | | | |
| gi\|3041715\|sp\|P2748 | PLASMA RETINOL- | 201 | 184/201 | 193/201 | e−106 |
| 5\|RETB_PIG | BINDING PROTEIN | | (91%) | (95%) | |
| | PRECURSOR (PRBP) | | | | |
| | (RBP) | | | | |
| gi\|1710096\|sp\|P0691 | PLASMA RETINOL- | 201 | 183/201 | 194/201 | e−106 |
| 2\|RETB_RABIT | BINDING PROTEIN | | (91%) | (96%) | |
| | PRECURSOR (PRBP) | | | | |
| | (RBP) | | | | |
| gi\|89271\|pir\|\|A3948 | plasma retinol- | 201 | 184/201 | 192/201 | e−106 |
| 6 | binding protein | | (91%) | (94%) | |
| | precursor - pig | | | | |

Table 58D lists the domain descriptions from DOMAIN analysis results against NOV58. This indicates that the NOV58 sequence has properties similar to those of other proteins known to contain this domain.

TABLE 58D
Domain Analysis of NOV58

gnl|Pfam|pfam00061, lipocalin, Lipocalin/cytosolic fatty-acid
binding protein family. Lipocalins are transporters for small
hydrophobic molecules, such as lipids, steroid hormones, bilins, and
retinoids. Alignment subsumes both the lipocalin and fatty acid
binding protein signatures from PROSITE. This is supported on
structural and functional grounds. Structure is an eight-stranded beta
barrel.
CD-Length = 145 residues, 100.0% aligned
Score = 102 bits (255), Expect = 2e−23

Vitamin A is mobilized from liver stores and transported in plasma in the form of the lipid alcohol retinol, bound to a specific transport protein, retinol-binding protein (RBP). A great deal is known about the chemical structure, metabolism, and biological roles of RBP. RBP is a single polypeptide chain with molecular weight close to 20,000. RBP interacts strongly with plasma prealbumin, and normally circulates in plasma as a 1:1 molar RBP-prealbumin complex. Both the primary and the tertiary structure of prealbumin are known, and the primary structure of RBP has recently been reported. Much information is available about the protein-protein and protein-ligand interactions that are involved in this transport system. Many clinical studies have examined the effects of a variety of diseases on the plasma levels of RBP and prealbumin in humans. Plasma RBP levels are low in patients with liver disease and are high in patients with chronic renal disease. These findings reflect the facts that RBP is produced in the liver and mainly catabolized in the kidneys. Delivery of retinol to extra-hepatic tissues appears to involve specific cell surface receptors for RBP. Vitamin A mobilization from the liver, and delivery to peripheral tissues, is highly regulated by factors that control the rates of RBP production and secretion. Retinol deficiency specifically blocks the secretion of RBP, so that plasma RBP levels fall and liver RBP levels rise. Injection of retinol into vitamin A-deficient rats stimulates the rapid secretion of RBP from the liver into the plasma (See Goodman D. S., 1980, Ann NY Acad Sci 348:378-90).

The disclosed NOV58 nucleic acid of the invention encoding a plasma retinol binding protein-like protein includes the nucleic acid whose sequence is provided in Table 58A or a fragment thereof. The invention also includes a mutant or variant nucleic acid any of whose bases may be changed from the corresponding base shown in Table 58A while still encoding a protein that maintains its plasma retinol binding protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 3 percent of the bases may be so changed.

The disclosed NOV58 protein of the invention includes the plasma retinol binding protein-like protein whose sequence is provided in Table 58B. The invention also includes a mutant or variant protein any of whose residues may be changed from the corresponding residue shown in Table 58B while still encoding a protein that maintains its plasma retinol binding protein-like activities and physiological functions, or a functional fragment thereof. In the mutant or variant protein, up to about 3 percent of the residues may be so changed.

The invention further encompasses antibodies and antibody fragments, such as F_{ab} or (F_{ab})₂, that bind immunospecifically to any of the proteins of the invention.

The above defined information for this invention suggests that this plasma retinol binding protein-like protein (NOV58) may function as a member of a “plasma retinol binding protein family”. Therefore, the NOV58 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV58 nucleic acids and proteins of the invention are useful in potential therapeutic applications implicated in diseases including but not limited to various pathologies and disorders as indicated below. For example, a cDNA encoding the plasma retinol binding protein-like protein (NOV58) may be useful in gene therapy, and the plasma retinol binding protein-like protein (NOV58) may be useful when administered to a subject in need thereof. By way of nonlimiting example, the compositions of the present invention will have efficacy for treatment of patients suffering from cervical dysplasias and cancer, breast cancer, phenylketonuria, liver diseases, kidney diseases, alzheimers, infection and inflammations, or other pathologies or conditions. The NOV58 nucleic acid encoding the plasma retinol binding protein-like protein of the invention, or fragments thereof, may further be useful in diagnostic applications, wherein the presence or amount of the nucleic acid or the protein are to be assessed.

NOV58 nucleic acids and polypeptides are further useful in the generation of antibodies that bind immuno-specifically to the novel NOV58 substances for use in therapeutic or diagnostic methods. These antibodies may be generated according to methods known in the art, using prediction from hydrophobicity charts, as described in the “Anti-NOVX Antibodies” section below. The disclosed NOV58 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV59

A disclosed NOV59 nucleic acid of 1647 nucleotides (also referred to as CG57566-01) encoding a HIV-1 inducer of short transcripts binding protein like protein-like protein is shown in Table 59A. The start and stop codons are in bold letters.

TABLE 59A
NOV59 nucleotide sequence.

| | |
|---|---|
| ATGGCCGGCGGCGTGGACGGCCCCATCGGGATCCCGTTCCCCGACCACAGCAGCGACATC | (SEQ ID NO:135) |
| CTGAGTGGGCTGAACGAGCAGCGGACGCAGGGCCTGCTGTGCGACGTGGTGATCCTGGTG | |
| GAGGGCCGCGAGTTCCCCACGCACCGCTCGGTGCTGGCCGCCTGCAGCCAGTACTTCAAG | |
| AAGCTGTTCACGTCGGGCGCCGTGGTGGACCAGCAGAACGTGTACGAGATCGACTTCGTC | |
| AGCGCCGAGGCGCTCACCGCGCTCATGGACTTCGCCTACACGGCCACGCTCACCGTCAGC | |
| ACAGCCAACGTGGGTGACATCCTCAGCGCCGCCCGCCTGCTGGAGATCCCCGCCGTGAGC | |
| CACGTGTGCGCCGACCTCCTGGACCGGCAGATCCTGGCGGCCGACGCGGGCGCCGACGCC | |
| GGGCAGCTGGACCTTGTAGATCAAATTGATCAGCGCAACCTCCTCCGCGCCAAGGAGTAC | |
| CTCGAGTTCTTCCAGAGCAACCCCATGAACAGCCTGCCCCCCGCGGCCGCCGCCGCCGCT | |
| GCCAGCTTCCCGTGGTCCGCCTTTGGGGCGTCCGATGATGACCTGGATGCCACCAAGGAG | |
| GCCGTGGCCGCCGCTGTGGCCGCCGTGGCCGCGGGCGACTGCAACGGCTTAGACTTCTAT | |
| GGGCCGGGCCCCCCGGCCGAGCGGCCCCCGACGGGGGACGGGGACGAGGGCGACAGCAAC | |
| CCGGGTCTGTGGCCAGAGCGGGATGAGGACGCCCCCACCGGGGGTCTCTTTCCGCCGCCG | |
| GTGGCCCCGCCGGCCGCCACGCAGAACGGCCACTACGGCCGCGGCGGAGAGGAGGAGGCC | |
| GCCTCGCTGTCGGAGGCGGCCCCCGAGCCGGGCGACTCTCCGGGCTTCCTGTCGGGAGAC | |
| AGCGACGAGGAGTCGCGGGCCGACGACAAGGGCGTCATGGACTACTACCTGAAGTACTTC | |
| AGCGGCGCCCACGACGGCGACGTCTACCCGGCCTGGTCGCAGAAGGTGGAGAAGAAGATC | |
| CGAGCCAAGGCCTTCCAGAAGTGCCCCATCTGCGAGAAGGTCATCCAGGGCGCCGGCAAG | |
| CTGCCGCGACACATCCGCACCCACACGGGCGAGAAGCCCTACGAGTGCAACATCTGCAAG | |
| GTCCGCTTCACCAGGCAGGACAAGCTGAAGGTGCACATGCGGAAGCACACGGGCGAGAAG | |
| CCGTACCTGTGCCAGCAGTGCGGCGCCGCCTTTGCCCACAACTACGACCTGAAGAACCAC | |
| ATGCTCGTGCACACGGGCCTGCGCCCCTACCAGTGCGACAGCTGCTGCAAGACCTTCGTC | |
| CGCTCCGACCACCTGCACAGACACCTCAAGAAAGACGGCTGCAACGGCGTCCCCTCGCGC | |
| CGCGGCCGCAAGCCCCGCGTCCGGGGCGGGGCGCCCGACCCCAGCCCGGGGGCCACCGCG | |
| ACCCCCGGCGCCCCCGCCCAGCCCAGCTCCCCCGACGCCCGGCGCAACGGCCAGGAGAAG | |
| CACTTTAAGGACGAGGACGAGGACGAGGACGTGGCCAGCCCCGACGGCTTGGGCCGGTTG | |
| AATGTAGCGGGCGCCGGTGGAGGAGGTGACAGCGGAGGTGGCCCCGGGGCCGCCACCGAC | |
| GGTAACTTCACAGCCGGACTCGCCTAA | |

In a search of public sequence databases, the NOV59 nucleic acid sequence has 1271 of 1560 bases (81%) identical to a gb:GENBANK-ID:AF097916|acc:AF097916.1 mRNA from Homo sapiens (Homo sapiens HIV-1 inducer of short transcripts binding protein (FBI1) mRNA, complete cds). Public nucleotide databases include all GenBank databases and the GeneSeq patent database.

The disclosed NOV59 polypeptide (SEQ ID NO:136) encoded by SEQ ID NO:135 has 548 amino acid residues and is presented in Table 59B using the one-letter amino acid code. Signal P, Psort and/o Hydropathy results predict that NOV59 has no signal peptide and is likely to be localized in the nucleus with a certainty of 0.8800.

TABLE 59B
Encoded NOV59 protein sequence.

| | |
|---|---|
| MAGGVDGPIGIPFPDHSSDILSGLNEQRTQGLLCDVVILVEGREFPTHRSVLAACSQYFK | (SEQ ID NO:136) |
| KLFTSGAVVDQQNVYEIDFVSAEALTALMDFAYTATLTVSTANVGDILSAARLLEIPAVS | |
| HVCADLLDRQILAADAGADAGQLDLVDQIDQRNLLRAKEYLEFFQSNPMNSLPPAAAAAA | |
| ASFPWSAFGASDDDLDATKEAVAAAVAAVAAGDCNGLDFYGPGPPAERPPTGDGDEGDSN | |
| PGLWPERDEDAPTGGLFPPPVAPPAATQNGHYGRGGEEEAASLSEAAPEPGDSPGFLSGD | |
| SDEESRADDKGVMDYYLKYFSGAHDGDVYPAWSQKVEKKIRAKAFQKCPICEKVIQGAGK | |
| LPRHIRTHTGEKPYECNICKVRFTRQDKLKVHMRKHTGEKPYLCQQCGAAFAHNYDLKNH | |
| MRVHTGLRPYQCDSCCKTFVRSDHLHRHLKKDGCNGVPSRRGRKPRVRGGAPDPSPGATA | |
| TPGAPAQPSSPDARRNGQEKHFKDEDEDEDVASPDGLGRLNVAGAGGGGDSGGGPGAATD | |
| GNFTAGLA | |

A search of sequence databases reveals that the NOV59 amino acid sequence has 344 of 458 amino acid residues (75%) identical to, and 362 of 458 amino acid residues (79%) similar to, the 584 amino acid residue ptnr:SPTREMBL-ACC:O95365 protein from Homo sapiens (Human) (HIV-1 INDUCER OF SHORT TRANSCRIPTS BINDING PROTEIN). Public amino acid databases include the GenBank databases, SwissProt, PDB and PIR.

NOV59 is expressed in at least tumor, inflammed, and brain. This information was derived by determining the tissue sources of the sequences that were included in the invention including but not limited to SeqCalling sources, Public EST sources, Literature sources, and/or RACE sources.

The disclosed NOV59 polypeptide has homology to the amino acid sequences shown in the BLASTP data listed in Table 59C.

TABLE 59C
BLAST results for NOV59

| Gene Index/ | | Length | Identity | Positives | |
|---|---|---|---|---|---|
| Identifier | Protein/Organism | (aa) | (%) | (%) | Expect |
| gi\|7705375\|ref\|NP_0 | HIV-1 inducer of | 584 | 548/584 | 548/584 | e−167 |
| 56982.1\| | short transcripts | | (93%) | (93%) | |
| (NM_015898) | binding protein | | | | |
| | [Homo sapiens] | | | | |
| gi\|16758916\|ref\|NP_{—} | leukemia/lymphoma | 569 | 477/579 | 489/579 | e−159 |
| 446454.1\| | related factor | | (82%) | (84%) | |
| (NM_054002) | [Rattus | | | | |
| | norvegicus] | | | | |
| gi\|6754572\|ref\|NP_0 | leukemia/lymphoma | 565 | 479/579 | 488/579 | e−125 |
| 34861.1\| | related factor | | (82%) | (83%) | |
| (NM_010731) | [Mus musculus] | | | | |
| gi\|3599513\|gb\|AAC35 | leukemia/lymohoma | 546 | 359/578 | 400/578 | 4e−13 |
| 368.1\|(AF086831) | related factor | | (62%) | (69%) | |
| | cLRF [Gallus | | | | |
| | gallus] | | | | |
| gi\|2145062\|gb\|AAB58 | TTF-I interacting | 590 | 293/297 | 295/297 | 4e−12 |
| 414.1\|(AF000561) | peptide 21; | | (98%) | (98%) | |
| | TIP21; | | | | |
| | Transcription | | | | |
| | Termination | | | | |
| | Factor I | | | | |
| | Interacting | | | | |
| | Peptide 21 [Homo | | | | |
| | sapiens] | | | | |

Tables 59D-E list the domain descriptions from DOMAIN analysis results against NOV59. This indicates that the NOV59 sequence has properties similar to those of other proteins known to contain this domain.

TABLE 59D
Domain Analysis of NOV59

gnl|Pfam|pfam00651, BTB, BTB/POZ domain. The BTB (for BR-C,
ttk and bab) or POZ (for Pox virus and Zinc finger) domain is
present near the N-terminus of a fraction of zinc finger
(pfam00096) proteins and in proteins that contain the pfam01344
motif such as Kelch and a family of pox virus proteins. The
BTB/POZ domain mediates homomeric dimerisation and in some
instances heteromeric dimerisation. The structure of the
dimerised PLZF BTB/POZ domain has been solved and consists of a
tightly intertwined homodimer. The central scaffolding of the
protein is made up of a cluster of alpha-helices flanked by short
beta-sheets at both the top and bottom of the molecule. POZ
domains from several zinc finger proteins have been shown to
mediate transcriptional repression and to interact with components
of histone deacetylase co-repressor complexes including N-CoR and
SMRT. The POZ or BTB domain is also known as BR-C/Ttk or ZiN
CD-Length = 114 residues, 100.0% aligned
Score = 122 bits (305), Expect = 7e−29

TABLE 59E
Domain Analysis of NOV59

gnl|Pfam|pfam00096, zf-C2H2, Zinc finger, C2H2 type.
The C2H2 zinc finger is the classical zinc finger domain.
The two conserved cysteines and histidines co-ordinate a
zinc ion. The following pattern describes the zinc finger.
#-X—C—X(1-5)-C—X3-#-X5-#-X2-H—X(3-6)-[H/C]
Where X can be any amino acid, and numbers in brackets
indicate the number of residues. The positions marked #
are those that are important for the stable fold of the zinc
finger. The final position can be either his or cys. The
C2H2 zinc finger is composed of two short beta strands
followed by an alpha helix. The amino terminal part of the
helix binds the major groove in DNA binding zinc fingers.
CD-Length = 23 residues, 100.0% aligned
Score = 38.5 bits (88), Expect = 0.001

The HIV-1 promoter directs the synthesis of two classes of transcripts, short, non-polyadenylated transcripts and full-length, polyadenylated transcripts. The synthesis of these transcripts is activated by a bipartite DNA element, the inducer of short transcripts or IST, located downstream of the HIV-1 transcriptional start site, while the synthesis of full-length transcripts is activated by the viral activator Tat. Tat binds to the RNA element TAR, which is encoded largely between the two IST half-elements. Upon activation by Tat, the synthesis of short RNAs is repressed. A factor called FBI-1 (for factor that binds to IST) whose binding to wild-type and mutated ISTs correlated well with the abilities of these ISTs to direct the synthesis of short transcripts was identified by Morrison, et al (See Morrison et al., Nucleic Acids Res Mar. 1, 1999: 1251-62). FBI-1 contains a POZ domain at its N-terminus and four Kruppel-type zinc fingers at its C-terminus. The C-terminus is sufficient for specific binding, and FBI-1 can form homomers through its POZ domain and, in vivo, through its zinc finger domain as well. In addition, FBI-1 associates with Tat, suggesting that repression of the short transcripts by Tat may be mediated through interactions between the two factors.

The disclosed NOV59 nucleic acid of the invention encoding a HIV-1 inducer of short transcripts binding protein like protein-like protein includes the nucleic acid whose sequence is provided in Table 59A or a fragment thereof. The invention also includes a mutant or variant nucleic acid any of whose bases may be changed from the corresponding base shown in Table 59A while still encoding a protein that maintains its HIV-1 inducer of short transcripts binding protein like protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 19 percent of the bases may be so changed.

The disclosed NOV59 protein of the invention includes the HIV-1 inducer of short transcripts binding protein like protein-like protein whose sequence is provided in Table 59B. The invention also includes a mutant or variant protein any of whose residues may be changed from the corresponding residue shown in Table 59B while still encoding a protein that maintains its HIV-1 inducer of short transcripts binding protein like protein-like activities and physiological functions, or a functional fragment thereof. In the mutant or variant protein, up to about 25 percent of the residues may be so changed.

The invention further encompasses antibodies and antibody fragments, such as F_{ab} or (F_{ab})₂, that bind immunospecifically to any of the proteins of the invention.

The above defined information for this invention suggests that this HIV-1 inducer of short transcripts binding protein like protein-like protein (NOV59) may function as a member of a “HIV-1 inducer of short transcripts binding protein like protein family”. Therefore, the NOV59 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV59 nucleic acids and proteins of the invention are useful in potential therapeutic applications implicated in diseases including but not limited to various pathologies and disorders as indicated below. For example, a cDNA encoding the HIV-1 inducer of short transcripts binding protein like protein-like protein (NOV59) may be useful in gene therapy, and the HIV-1 inducer of short transcripts binding protein like protein-like protein (NOV59) may be useful when administered to a subject in need thereof. By way of nonlimiting example, the compositions of the present invention will have efficacy for treatment of patients suffering from Human Immunodeficiency Virus/Acquired immune deficiency syndrome, cancer, and inflammatory diseases, or other pathologies or conditions. The NOV59 nucleic acid encoding the HIV-1 inducer of short transcripts binding protein like protein-like protein of the invention, or fragments thereof, may further be useful in diagnostic applications, wherein the presence or amount of the nucleic acid or the protein are to be assessed.

NOV59 nucleic acids and polypeptides are further useful in the generation of antibodies that bind immuno-specifically to the novel NOV59 substances for use in therapeutic or diagnostic methods. These antibodies may be generated according to methods known in the art, using prediction from hydrophobicity charts, as described in the “Anti-NOVX Antibodies” section below. The disclosed NOV59 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV60

NOV60 includes three beta tectorin-like proteins disclosed below. The disclosed sequences have been named NOV60a, NOV60b and NOV60c.

NOV60a

A disclosed NOV60a nucleic acid of 1011 nucleotides (also referred to as CG57574-01) encoding a beta tectorin-like protein is shown in Table 60A. The start and stop codons are in bold letters.

TABLE 60A
NOV60a nucleotide sequence.

| | |
|---|---|
| GATCGAGGCTCAGGCCCTGGAAGGACCGTAAACATTTGGCCAGCTTGGTTTGGATACCTG | (SEQ ID NO:137) |
| GCAGAGACCAGGTTCTGAGAAGCAATGGTGACGAAGGCCTTTGTCTTGTTGGCCATCTTT | |
| GCAGAAGCCTCTGCAAAATCGTGTGCTCCAAATAAAGCAGATGTCATTCTTGTGTTTTGC | |
| TATCCCAAAACCATCATCACCAAAATCCCCGAGTGTCCCTATGGATGGGAAGTTCATCAG | |
| CTGGCCCTCGGAGGGCTGTGTTACAATGGGGTCCACGAAGGAGGTTACTACCAATTTGTG | |
| ATCCCAGATTTATCACCTAAAAACAAGTCCTATTGTGGAACCCAGTCTGAGTACAAGCCA | |
| CCTATCTATCACTTCTACAGTCACATCGTTTCCAATGACACCACAGTGATTGTAAAAAAC | |
| CAGCCTGTCAACTACTCCTTCTCCTGCACCTACCACTCCACCTACTTGGTGAACCAGGCT | |
| GCCTTTGACCAGAGTGTCAATTTCCTTCCAAAGAATGCCAAGTTCTCCATCAAGAAAGAA | |
| GCTCCCTTTGTCCTGGAGGCATCCGAAATCGGTTCAGATCTGTTTGCAGGAGTGGAAGCC | |
| AAAGGGTTAAGCATTAGGTTTAAAGTGGTCTTGAACAGCTGTTGGGCCACCCCCTCGGCT | |
| GACTTCATGTATCCCTTGCAGTGGCAGCTGATCAACAAGGGCTGCCCCACGGATGAAACC | |
| GTCCTCGTGCATGAGAATGGGAGAGATCACAGGGCAACCTTCCAATTCAATGCTTTCCGG | |
| TTCCAGAACATCCCCAAACTCTCCAAGGTGTGGTTACACTGTGAGACGTTCATCTGCGAC | |
| AGTGAGAAACTCTCCTGCCCAGTGACCTGCGATAAACGGAAGCGCCTCCTGCGAGACCAG | |
| ACCGGGGGAGTCCTGGTCGTGGAGCTCTCCCTGCGGAATGTTCTCCACCACCTCATCATG | |
| ATGTTGGGGATTTGTGCCGTGTTTAGGAGTTAGCCAGGCAGCTGCCGCTCCTCCACCCA | |
| CAATAG | |

In a search of public sequence databases, the NOV60a nucleic acid sequence, located on chromsome 10 has 428 of 496 bases (86%) identical to a gb:GENBANK-ID:MMBETATEC|acc:X99806.2 mRNA from Mus musculus (Mus musculus mRNA for beta tectorin). Public nucleotide databases include all GenBank databases and the GeneSeq patent database.

The disclosed NOV60a polypeptide (SEQ ID NO:138) encoded by SEQ ID NO:137 has 300 amino acid residues and is presented in Table 60B using the one-letter amino acid code. Signal P, Psort and/or Hydropathy results predict that NOV60a has a signal peptide and is likely to be localized plasma membrane with a certainty of 0.6850. The most likely cleavage site for a NOV60a peptide is between amino acids 17 and 18.

TABLE 60B
Encoded NOV60a protein sequence.

| | |
|---|---|
| MVTKAFVLLAIFAEASAKSCAPNKADVILVFCYPKTIITKIPECPYGWEVHQLALGGLCY | (SEQ ID NO:138) |
| NGVHEGGYYQFVIPDLSPKNKSYCGTQSEYKPPIYHFYSHIVSNDTTVIVKNQPVNYSFS | |
| CTYHSTYLVNQAAFDQSVNFLPKNAKFSIKKEAPFVLEASEIGSDLFAGVEAKGLSIRFK | |
| VVLNSCWATPSADFMYPLQWQLINKGCPTDETVLVHENGRDHRATFQFNAFRFQNIPKLS | |
| KVWLHCETFICDSELKSCPVTCDKRKRLLRDQTGGVLVVELSLRNVLHHLIMMLGICAVL | |

A search of sequence databases reveals that the NOV60a amino acid sequence has 149 of 174 amino acid residues (85%) identical to, and 155 of 174 amino acid residues (89%) similar to, the 329 amino acid residue ptnr:SPTREMBL-ACC:O08524 protein from Mus musculus (Mouse) (BETA TECTORIN). Public amino acid databases include the GenBank databases, SwissProt, PDB and PIR.

NOV60a is predicted to be expressed in the following tissues because of the expression pattern of (GENBANK-ID: gb:GENBANK-ID:MMBETATEC|acc:X99806.2) a closely related Mus musculus mRNA for beta tectorin homolog in species Mus musculus: cochleae. This information was derived by determining the tissue sources of the sequences that were included in the invention including but not limited to SeqCalling sources, Public EST sources, Literature sources, and/or RACE sources.

NOV60b

A disclosed NOV60b nucleic acid of 1012 nucleotides (also referred to as CG57574-02) encoding a beta tectorin-like protein is shown in Table 60C. The start and stop codons are in bold letters.

TABLE 60C
NOV60b nucleotide sequence.

(SEQ ID NO:139)

AGAGACCAGGTTCTGAGAAGCAATGGTGACGAAGGCCTTTGTCTTGTTGGCCATCTTTGCAGAAGCCTCT
GCAAAATCGTGTGCTCCAAATAAAGCAGATGTCATTCTTGTGTTTTGCTATCCCAAAACCATCATCACCA
AAATCCCCGAGTGTCCCTATGGATGGGAAGTTCATCAGCTGGCCCTCGGAGGGCTGTGTTACAATGGGGT
CCACGAAGGAGGTTACTACCAATTTGTGATCCCAGATTTATCACCTAAAAACAAGTCCTATTGTGGAACC
CAGTCTGAGTACAAGCCACCTATCTATCACTTCTACAGTCACATCGTTTCCAATGACGCCACAGTGATTG
TAAAAAACCAGCCTGTCAACTACTCCTTCTCCTGCACCTACCACTCCACCTACTTGGTGAACCAGGCTGC
CTTTGACCAGAGAGTGGCCACTGTTCACGTGAAGAACGGGAGCATGGGCACATTTGAGAGCCAACTGTCT
CTCAACTTCTACACTAATGCCAAGTTCTCCATCAAGAAAGAAGCTCCCTTTGTCCTGGAGGCATCCGAAA
TCGGTTCAGATCTGTTTGCAGGAGTGGAAGCCAAAGGGTTAAGCATTAGGTTTAAAGTGGTCTTGAACAG
CTGTTGGGCCACCCCCTCGGCTGACTTCATGTATCCCTTGCAGTGGCAGCTGATCAACAAGGGCTGCCCC
ACGGATGAAACCGTCCTCGTGCATGAGAATGGGAGAGATCACAGGGCAACCTTCCAATTCAATGCTTTCC
GGTTCCAGAACATCCCCAAACTCTCCAAGGTGTGGTTACACTGTGAGACGTTCATCTGCGACAGTGAGAA
ACTCTCCTGCCCAGTGACCTGCGATAAACGGAAGCGCCTCCTGCGAGACCAGACCGGGGGAGTCCTGGTC
GTGGAGCTCTCCCTGCGGAGCAGGGGATTTTCCAGTCTCTATAGCTTCTCAGATGTTCTCCACCACCTCA
TCATGATGTTGGGGATTTGTGCCGTGTTATAG

In a search of public sequence databases, the NOV60b nucleic acid sequence, located on chromsome 10 has 887 of 1012 bases (87%) identical to a gb:GENBANK-ID:MMBETATEC|acc:X99806.2 mRNA from Mus musculus (Mus musculus mRNA for beta tectorin). Public nucleotide databases include all GenBank databases and the GeneSeq patent database.

The disclosed NOV60b polypeptide (SEQ ID NO:140) encoded by SEQ ID NO:139 has 329 amino acid residues and is presented in Table 60D using the one-letter amino acid code. Signal P, Psort and/or Hydropathy results predict that NOV60b has a signal peptide and is likely to be localized extracellularly with a certainty of 0.6850. The most likely cleavage site for a NOV60b peptide is between amino acids 17 and 18.

TABLE 60D
Encoded NOV60b protein sequence.

| | |
|---|---|
| MVTKAFVLLAIFAEASAKSCAPNKADVILVFCYPKTIITKIPECPYGWEVHQLALGGLCY | (SEQ ID NO:140) |
| NGVHEGGYYQFVIPDLSPKNKSYCGTQSEYKPPIYHFYSHIVSNDATVIVKNQPVNYSFS | |
| CTYHSTYLVNQAAFDQRVATVHVKNGSMGTFESQLSLNFYTNAKFSIKKEAPFVLEASEI | |
| GSDLFAGVEAKGLSIRFKVVLNSCWATPDADFMYPLQWQLINKGCPTDETVLVHENGRDH | |
| RATFQFNAFRFQNIPKLSKVWLHCETFICDSELKSCPVTCDKRKRLLRDQTGGVLVVELS | |
| LRSRGFSSLYSFSDVLHHLIMMLGICAVL | |

A search of sequence databases reveals that the NOV60b amino acid sequence has have 310 of 329 amino acid residues (94%) identical to, and 317 of 329 amino acid residues (96%) similar to, the 329 amino acid residue ptnr:SPTREMMBL-ACC:O08524 protein from Mus musculus (Mouse) (BETA TECTORIN). Public amino acid databases include the GenBank databases, SwissProt, PDB and PIR.

NOV60b is predicted to be expressed in at least the ear. This information was derived by determining the tissue sources of the sequences that were included in the invention including but not limited to SeqCalling sources, Public EST sources, Literature sources, and/or RACE sources.

NOV60c

A disclosed NOV60c nucleic acid of 1012 nucleotides (also referred to as CG57574-02) encoding a beta tectorin-like protein is shown in Table 60E. The start and stop codons are in bold letters.

TABLE 60E
NOV60c nucleotide sequence.

| | |
|---|---|
| AGAGACCAGGTTCTGAGAAGCAATGGTGACGAAGGCCTTTGTCTTGTTGGCCATCTTTGC | (SEQ ID NO:141) |
| AGAAGCCTCTGCAAAATCGTGTGCTCCAAATAAAGCAGATGTCATTCTTGTGTTTTGCTA | |
| TCCCAAAACCATCATCACCAAAATCCCCGAGTGTCCCTATGGATGGGAAGTTCATCAGCT | |
| GGCCCTCGGAGGGCTGTGTTACAATGGGGTCCACGAAGGAGGTTACTACCAATTTGTGAT | |
| CCCAGATTTATCACCTAAAAACAAGTCCTATTGTGGAACCCAGTCTGAGTACAAGCCACC | |
| TATCTATCACTTCTACAGTCACATCGTTTCCAATGACACCACAGTGATTGTAAAAAACCA | |
| GCCTGTCAACTACTCCTTCTCCTGCACCTACCACTCCACCTACTTGGTGAACCAGGCTGC | |
| CTTTGACCAGAGAGTGGCCACTGTTCACGTGAAGAACGGGAGCATGGGCACATTTGAGAG | |
| CCAACTGTCTCTCAACTTCTACACTAATGCCAAGTTCTCCATCAAGAAAGAAGCTCCCTT | |
| TGTCCTGGAGGCATCGGAAATCGGTTCAGATCTGTTTGCAGGAGTGGAAGCCAAAGGGTT | |
| AAGCATTAGGTTTAAAGTGGTCTTGAACAGCTGTTCGGCCACCCCCTCGGCTGACTTCAT | |
| GTATCCCTTCCAGTGGCAGCTGATCAACAAGGGCTGCCCCACGGATCAAACCGTCCTCGT | |
| GCATGAGAATGGGAGAGATCACAGGGCAACCTTCCAATTCAATGCTTTCCGGTTCCAGAA | |
| CATCCCCAAACTCTCCAAGGTGTGGTTACACTGTGAGACGTTCATCTGCGACAGTGAGAA | |
| ACTCTCCTGCCCAGTGACCTGCGATAAACGGAAGCGCCTCCTGCGAGACCAGACCGGGGG | |
| AGTCCTGGTCGTGGAGCTCTCCCTGCGGAGCAGGGGATTTTCCAGTCTCTATAGCTTCTC | |
| AGATGTTCTCCACCACCTCATCATGATGTTGGGGATTTGTGCCGTGTTATAG | |

In a search of public sequence databases, the NOV60c nucleic acid sequence, located on chromsome 10 has 887 of 1012 bases (87%) identical to a gb:GENBANK-ID:MMBETATEC|acc:X99806.2 mRNA from Mus musculus (Mus musculus mRNA for beta tectorin). Public nucleotide databases include all GenBank databases and the GeneSeq patent database.

The disclosed NOV60c polypeptide (SEQ ID NO:142) encoded by SEQ ID NO:141 has 329 amino acid residues and is presented in Table 60F using the one-letter amino acid code. Signal P, Psort and/or Hydropathy results predict that NOV60c has a signal peptide and is likely to be localized plasma membrane with a certainty of 0.6850. The most likely cleavage site for a NOV60c peptide is between amino acids 17 and 18.

TABLE 60F
Encoded NOV60c protein sequence.

| | |
|---|---|
| MVTKAFVLLAIFAEASAKSCAPNKADVILVFCYPKTIITKIPECPYGWEVHQLALGGLCY | (SEQ ID NO:142) |
| NGVHEGGYYQFVIPDLSPKNKSYCGTQSEYKPPIYHFYSHIVSNDTTVIVKNQPVNYSFS | |
| CTYHSTYLVNQAAFDQRVATVHVKNGSMGTFESQLSLNFYTNAKFSIKKEAPFVLEASEI | |
| GSDLFAGVEAKGLSIRFKVVLNSCWATPSADFMYPLQWQLINKGCPTDETVLVHENGRDH | |
| RATFQFNAFRFQNIPKLSKVWLHCETFICDSEKLSCPVTCDKRKRLLRDQTGGVLVVELS | |
| LRSRGFSSLYSFSDVLHHLIMMLGICAVL | |

A search of sequence databases reveals that the NOV60c amino acid sequence has 310 of 329 amino acid residues (94%) identical to, and 317 of 329 amino acid residues (96%) similar to, the 329 amino acid residue ptnr:SPTPEMBL-ACC:O08524 protein from Mus musculus (Mouse) (BETA TECTORIN)Public amino acid databases include the GenBank databases, SwissProt, PDB and PIR.

NOV60c is predicted to be expressed in the following tissues because of the expression pattern of (GENBANK-ID: gb:GENBANK-ID:MMBETATEC|acc:X99806.2) a closely related Mus musculus mRNA for beta tectorin homolog in species Mus musculus cochleae. This information was derived by determining the tissue sources of the sequences that were included in the invention including but not limited to SeqCalling sources, Public EST sources, Literature sources, and/or RACE sources.

The disclosed NOV60c polypeptide has homology to the amino acid sequences shown in the BLASTP data listed in Table 60G.

TABLE 60G
BLAST results for NOV60c

| Gene Index/ | | Length | Identity | Positives | |
|---|---|---|---|---|---|
| Identifier | Protein/Organism | (aa) | (%) | (%) | Expect |
| gi\|17158035\|ref\|NP_{—} | tectorin beta | 329 | 295/330 | 297/330 | e−167 |
| 478129.1\| | [Homo sapiens] | | (89%) | (89%) | |
| (NM_058222 | | | | | |
| gi\|7363457\|ref\|NP_0 | tectorin beta; | 329 | 280/330 | 289/330 | e−159 |
| 33374.2\| | [b]-tectorin [Mus | | (84%) | (86%) | |
| (NM_009348) | musculus] | | | | |
| gi\|1729889\|sp\|P5409 | BETA-TECTORIN | 329 | 220/330 | 260/330 | e−125 |
| 7\|TECB_CHICK | PRECURSOR | | (66%) | (78%) | |
| gi\|13385494\|ref\|NP_{—} | RIKEN cDNA | 531 | 63/208 | 99/208 | 4e−13 |
| 080265.1\| | 2310037I18 [Mus | | (30%) | (47%) | |
| (NM_025989) | musculus] | | | | |
| gi\|12844889\|dbj\|BAB | homolog to | 573 | 62/200 | 96/200 | 4e−12 |
| 26538.1\|(AK009843) | PANCREATIC | | (31%) | (48%) | |
| | SECRETORY GRANULE | | | | |
| | MEMBRANE MAJOR | | | | |
| | GLYCOPROTEIN GP2 | | | | |
| | PRECURSOR | | | | |
| | (PANCREATIC | | | | |
| | ZYMOGEN GRANULE | | | | |
| | MEMBRANE PROTEIN | | | | |
| | GP-2) | | | | |
| | (ZAP75)-putative | | | | |
| | [Mus musculus] | | | | |

Table 60H lists the domain descriptions from DOMAIN analysis results against NOV60c. This indicates that the NOV60c sequence has properties similar to those of other proteins known to contain this domain.

TABLE 60H
Domain Analysis of NOV60c

gnl|Smart|smart00241, ZP, Zona pellucida (ZP)
domain; ZP proteins are responsible for sperm-adhesion
fo the zona pellucida. ZP domains are also present in
multidomain transmembrane proteins such as glycoprotein
GP2, uromodulin and TGF-beta receptor type III
(betaglycan).
CD-Length = 253 residues, 98.0% aligned
Score = 119 bits (297), Expect = 3e−28

Legan et al. (1997) cloned mouse alpha- and beta-tectorins. The mouse beta-tectorin gene encodes a 320-amino acid protein containing a hydrophobic secretory signal sequence and 4 potential N-glycosylation sites. Both alpha- and beta-tectorin contain a zona pellucida domain, but otherwise are not homologous.

To identify genes expressed in the vertebrate inner ear, Fleller et al. (1998) established an assay that allowed rapid analysis of the differential expression pattern of mRNAs derived from an auditory epithelium-specific cDNA library. They performed subtractive hybridization to create an enriched probe, which was then used to screen the cDNA library. After digoxigenin-labeled antisense cRNAs had been transcribed from hybridization-positive clones, they conducted in situ hybridization on slides bearing cryosections of late embryonic chicken heads, bodies, and cochleas. They found 12 proteins whose mRNAs were specifically or highly expressed in the chicken's inner ear; the remainder encoded proteins that occur more widely. They identified proteins that had previously been described as expressed in the inner ear, such as beta-tectorin, calbindin (CALB1; 114050), and type II collagen (COL2A1; 120140). A second group of proteins abundant in the inner ear included 5 additional types of collagen. A third group, including COCH5B2 (COCH; 603196) and ear-specific connexin, comprised the proteins whose human equivalents are candidates to account for hearing disorders. This last group also included proteins expressed in 2 cells types unique to the inner ear, homogene cells and cells of the tegmentum vasculosum.

The disclosed NOV60c nucleic acid of the invention encoding a beta tectorin-like protein includes the nucleic acid whose sequence is provided in Table 60A or a fragment thereof. The invention also includes a mutant or variant nucleic acid any of whose bases may be changed from the corresponding base shown in Table 60A while still encoding a protein that maintains its beta tectorin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 13 percent of the bases may be so changed.

The disclosed NOV60 protein of the invention includes the beta tectorin-like protein whose sequence is provided in Table 60B. The invention also includes a mutant or variant protein any of whose residues may be changed from the corresponding residue shown in Table 60B while still encoding a protein that maintains its beta tectorin-like activities and physiological functions, or a functional fragment thereof. In the mutant or variant protein, up to about 6 percent of the residues may be so changed.

The invention further encompasses antibodies and antibody fragments, such as F_{ab} or (F_{ab})₂, that bind immunospecifically to any of the proteins of the invention.

The above defined information for this invention suggests that this beta tectorin-like protein (NOV60) may function as a member of a “beta tectorin family”. Therefore, the NOV60 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV60 nucleic acids and proteins of the invention are useful in potential therapeutic applications implicated in diseases including but not limited to various pathologies and disorders as indicated below. For example, a cDNA encoding the beta tectorin-like protein (NOV60) may be useful in gene therapy, and the beta tectorin-like protein (NOV60) may be useful when administered to a subject in need thereof. By way of nonlimiting example, the compositions of the present invention will have efficacy for treatment of patients suffering from hearing loss, or other pathologies or conditions. The NOV60 nucleic acid encoding the replacement-like protein of the invention, or fragments thereof, may further be useful in diagnostic applications, wherein the presence or amount of the nucleic acid or the protein are to be assessed.

NOV60 nucleic acids and polypeptides are further useful in the generation of antibodies that bind immuno-specifically to the novel NOV60 substances for use in therapeutic or diagnostic methods. These antibodies may be generated according to methods known in the art, using prediction from hydrophobicity charts, as described in the “Anti-NOVX Antibodies” section below. The disclosed NOV60 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV61

A disclosed NOV61 nucleic acid of 3802 nucleotides (also referred to as CG57505-01) encoding a KIAA1125-like protein is shown in Table 61A. The start and stop codons are in bold letters.

TABLE 61A
NOV61 nucleotide sequence.

| | |
|---|---|
| GGGCAGCCAATCGGGGATGAGCTTTTATTAGGCGGCCAGATCATCAGCCGAAGTGCCAAA | (SEQ ID NO:143) |
| CCCTTTTTCTGTCAGAACTAGGAGCCTGTCCTCCATGTTTTATAAGTATTGACATTACAC | |
| AGTGTTAACAATGCATCCACAGAGCTTGGCTGAAGAGGAAATAAAAACAGAACAGGAGGT | |
| GGTAGAGGGCATGGATATCTCTACTCGCTCCAAAGATCCTGGCTCTGCAGAGAGAACAGC | |
| CCAGAAAAGAAAGTTCCCCAGCCCTCCACATTCTTCCAATGGCCACTCGCCGCAGGACAC | |
| ATCAACAAGCCCCATTAAAAAGAAAAAGAAACCTGGCTTACTGAACAGTAACAATAAGGA | |
| GCAGTCAGAACTAAGACATGGTCCGTTTTACTATATGAAGCAGCCACTCACCACAGACCC | |
| TGTTGATGTTGTACCCCAGGATGGACGGAATGATTTCTACTGCTGGGTTTGTCACCGGGA | |
| AGGCCAAGTCCTTTGCTCTGAGCTCTGTCCCCGGGTTTATCACGCTAACTGTCTGAGACT | |
| GACATCGGAACCAGAGGGGGACTGGTTTTGTCCTGAATGTGAGAAAATTACAGTAGCAGA | |
| ATGCATCGAGACCCAGAGTAAAGCCATCACAATGCTCACCATTGAACAGTTATCCTACCT | |
| GCTCAAGTTTGCCATTCAGAAAATGAAACAGCCAGGGACAGATCCATTCCAGAAGCCCGT | |
| TCCATTGGAACAGCACCCTGACTATGCGGAATACATCTTCCATCCAATGGACCTTTGTAC | |
| ATTGGAAAAGAATGCGAAAAAGAAAATGTATGGCTGCACAGAAGCCTTCCTGGCTGATGC | |
| AAAGTGGATTTTGCACAACTGCATCATTTATAATGGGCGAAATCACAAATTGACCCAAAT | |
| AGCGAAAGTAGTCATCAAAATCTGTGAACATGAGATGAATGAAATCGAAGTATGTCCAGA | |
| ATGTTATCTAGCTGCTTGCCAAAAACGAGATAACTGGTTTTGTGAGCCTTGTAGCAATCC | |
| ACATCCTTTGGTCTGGGCCAAACTGAAGGGGTTTCCATTCTGGCCTGCAAAAGCTCTAAG | |
| GGATAAAGACGGGCAGGTCGATGCCCGATTCTTTGGACAACATGACACGGCCTGGGTTCC | |
| AATAAATAATTGCTACCTCATGTCTAAAGAAATTCCTTTTTCTGTGAAAAAGACTAAGAG | |
| CATCTTCAACAGTGCCATGCAAGAGATGGAGGTTTACGTGGAGAACATCCGCAGGAAGTT | |
| TGGGGTTTTTAATTACTCTCCATTTAGGACACCCTACACACCCAACAGCCAGTATCAAAT | |
| GCTGCTCGATCCCACCAACCCCAGCGCCGGCACTGCCAAGATAGACAAGCAGGAGAAGGT | |
| CAAGCTCAACTTTGACATGACGGCATCCCCCAAGATCCTGATGAGCAAGCCTGTGCTGAG | |
| TGGGGGCACAGGCCGCCGGATTTCCTTGTCGGATATGCCGCGCTCCCCCATGAGCACAAA | |
| CTCTTCTGTGCACACGGGCTCCGACGTGGAGCAGGATGCTGAGAAGAAGGCCACGTCGAG | |
| CCACTTCAGTGCGAGCGAGGAGTCCATGGACTTCCTGGATAAGAGCACAGCTTCACCAGC | |
| CTCCACCAAGACGGGACAAGCAGGGAGTTTATCCGGCAGCCCAAAGCCCTTCTCTCCTCA | |
| ACTGTCAGCTCCTATCACGACGAAAACGGACAAAACCTCCACCACCGGCAGCATCCTGAA | |
| TCTTAACCTGGATCGAAGCAAAGCTGAGATGGATTTGAAGGAGCTGAGCGAGTCGGTCCA | |
| GCAACAGTCCACCCCTGTTCCTCTCATCTCTCCCAAGCGCCAGATTCGTAGCAGGTTCCA | |
| GCTGAATCTTGACAAGACCATAGAGAGTTGCAAAGCACAATTAGGCATAAATGAAATCTC | |
| GGAAGATGTCTATACGGCCGTAGAGCACAGCGATTCGGAGGATTCTCAGAAGTCAGATAG | |
| TAGCGATAGTGAGTATATCAGTGATGATGAGCAGAAGTCTAAGAACGAGCCAGAAGACAC | |
| AGAGGACAAAGAAGGTTGTCAGATGGACAAAGAGCCATCTGCTGTTAAAAAAAAGCCCAA | |
| GCCTACAAACCCAGTGGAGATTAAAGAGGAGCTGAAAAGCACGTCACCAGCCAGCGAGAA | |
| GGCAGACCCTGGAGCAGTCAAGGACAAGGCCAGCCCTGAGCCTGAGAAGGACTTTTCCGA | |
| AAAGGCAAAACCTTCACCTCACCCCATAAAGGATAAACTGAAGGGAAAAGATGAGACGGA | |
| TTCCCCAACAGTCCATTTGGGCCTGGACTCTGATTCAGAGAGCGAACTTGTCATAGATTT | |
| AGGAGAAGACCATTCTGGGCCCGAGGGTCGAAAAAATAAGAAGGAACCCAAAGAACCATC | |
| TCCCAAACAGGATGTTGTAGGTAAAACTCCACCATCCACGACGGTGGGCAGCCATTCTCC | |
| CCCGGAAACACCGGTGCTCACCCGCTCTTCCGCCCAAACTTCCGCGGCTGGCGCCACAGC | |
| CACCACCAGCACGTCCTCCACGGTCACCGTCACGGCCCCGGCCCCCGCCGCCACAGGAAG | |
| CCCAGTGAAAAAGCAGAGGCCGCTTTTACCGAAGGAGACTGCCCCGGCCGTGCAGCGGGT | |
| CGTGTGGAACTCATCAAGTAAGTTTCAAACGTCCTCCCAAAAGTGGCACATGCAGAAGAT | |
| GCAGCGTCAGCAGCAGCAGCAGCAGCAGCAAAACCAGCAGCAGCAGCCTCAGTCTTCCCA | |
| GGGGACGAGATATCAGACCAGACAGGCTGTGAAAGCTGTCCAGCAGAAGGAGATCACACA | |
| GAGCCCATCCACGTCCACCATCACCCTGGTGACCAGCACACAGTCATCGCCCCTGGTCAC | |
| CAGCTCGGGGTCCATGAGCACCCTTGTGTCCTCAGTCAACCCTGACCTGCCCATCGCCAC | |
| TGCCTCAGCTGATGTCGCCGCTGATATTGCCAAGTACACTAGCAAAATGATGGATGCAAT | |
| AAAAGGAACAATGACAGAAATATACAACGATCTTTCTAAAAACACTACTGGAAGCACAAT | |
| AGCTGAGATTCGCAGGCTGAGGATCGAGATAGAGAAGCTCCAGTGGCTGCACCAGCAAGA | |
| GCTCTCCGAAATGAAACACAACTTAGAGCTGACCATGGCGGACATGCGGCAGAGCCTGGA | |
| GCAGGAGCGGGACCGGCTCATCGCCGAGGTGAAGAAGCAGCTGGAGTTCGAGAAGCAGCA | |
| GGCGGTGGATGAGACCAAGAAGAAGCAGTGGTGCGCCAACTGCAAGAAGGAGGCCATCTT | |
| TTACTGCTGTTGGAACACTAGCTACTGTGACTACCCCTGCCAGCAAGCCCACTGGCCTGA | |
| GCACATGAAGTCCTGCACCCAGTCAGCTACTGCTCCTCAGCAGGAAGCGGATGCTGAGGT | |
| GAACACAGAAACACTAAATAAGTCCTCCCAGGGGAGCTCCTCGAGCACACAATCAGCACC | |
| TTCAGAAACGGCCAGCGCCTCCAAAGAGAAGGAGACGTCAGCTGAGAAAAGCAAGGACAG | |
| TGGCTCGACCCTTGACCTTTCTGGCTCCAGAGAGACGCCCTCCTCCATTCTCTTAGGCTC | |
| CAACCAAGGCTCTGACCATTCCCGGAGTAATAAATCCAGTTGGAGCAGCAGTGATGAGAA | |
| GAGGGGATCGACACGTTCCGATCACAACACCAGTACCAGCACGAAGAGCCTCCTCCCGAA | |
| AGAGTCTCGGCTGGACACCTTCTGGGACTAGCAGTGAATCGGGACACAAACCACCCACCC | |
| CATTGGGAGAAAAACCCAGACGCCAGGAAAAGAAGAAACAACAAAGGCAGGAGAACAGCC | |
| ACTTTCAGACTTGAAAATGACAAAACCCTCAGTTGAGCCTGACCCCCCGCCGCGGGGGCT | |
| GCTACACTA | |

In a search of public sequence databases, the NOV61 nucleic acid sequence, located on chromsome 20 has 3797 of 3827 bases (99%) identical to a gb:GENBANK-ID:AB03295|acc:AB032951.1 mRNA from Homo sapiens (Homo sapiens mRNA for KIAA1125 protein, partial cds). Public nucleotide databases include all GenBank databases and the GeneSeq patent database.

The disclosed NOV61 polypeptide (SEQ ID NO:144) encoded by SEQ ID NO:143 has 1206 amino acid residues and is presented in Table 61B using the one-letter amino acid code. Signal P, Psort and/or Hydropathy results predict that NOV61 has no signal peptide and is likely to be localized in the nucleus with a certainty of 0.7000.

TABLE 61B
Encoded NOV61 protein sequence.

| | |
|---|---|
| MHPQSLAEEEIKTEQEVVEGMDISTRSKDPGSAERTAQKRKFPSPPHSSNGHSPQDTSTS | (SEQ ID NO:144) |
| PIKKKKKPGLLNSNNKEQSELRHGPFYYMKQPLTTDPVDVVPQDGRNDFYCWVCHREGQV | |
| LCCELCPRVYHAKCLRLTSEPEGDWFCPECEKITVAECIETQSKAMTMLTIEQLSYLLKF | |
| AIQKMKQPGTDAFQKPVPLEQHPDYAEYIFHPMDLCTLEKNAKKKMYGCTEAFLADAKWI | |
| LHNCIIYNGGNHKLTQIAKVVIKICEHEMNEIEVCPECYLAACQKRDNWFCEPCSNPHPL | |
| VWAKLKGFPFWPAKALRDKDGQVDARFFGQHDRAWVPINNCYLMSKEIPFSVKKTKSIFN | |
| SAMQEMEVYVENIRRKFGVFNYSPFRTPYTPNSQYQMLLDPTNPSAGTAKIDKQEKVKLN | |
| FDMTASPKILMSKPVLSGGTGRRISLSDMPRSPMSTNSSVHTGSDVEQDAEKKATSSHFS | |
| ASEESMDFLDKSTASPASTKTGQAGSLSGSPKPFSPQLSAPITTKTKDTSTTGSILNLNL | |
| DRSKAEMDLKELSESVQQQSTPVPLISPKRQIRSRFQLNLDKTIESCKAQLGINEISEDV | |
| YTAVEHSDSEDSEKSDSSDSEYISDDEQKSKNEPEDTEDKEGCQMDKEPSAVKKKPKPTN | |
| PVEIKEELKSTSPASEKADPGAVKDKASPEPEKDFSEKAKPSPHPIKDKLKGKDETDSPT | |
| VHLGLDSDSESELVIDLGEDHSGREGRKNKKEPKEPSPKQDVVGKTPPSTTVGSHSPPET | |
| PVLTRSSAQTSAAGATATTSTSSTVTVTAPAPAATGSPVKKQRPLLPKETAPAVQRVVWN | |
| SSSKFQTSSQKWHMQKMQRQQQQQQQQNQQQQPQSSQGTRYQTRQAVKAVQQKEITQSPS | |
| TSTITLVTSTQSSPLVTSSGSMSTLVSSVNADLPIATASADVAADIAKYTSKMMDAIKGT | |
| MTEIYNDLSKNTTGSTIAEIRRLRIEIEKLQWLHQQELSEMKHNLELTMAEMRQSLEQER | |
| DRLIAEVKKQLELEKQQAVDETKKKQWCANCKKEAIFYCCWNTSYCDYPCQQAHWPEHMK | |
| SCTQSATAPQQEADAEVNTETLNKSSQGSSSSTQSAPSETASASKEKETSAEKSKESGST | |
| LDLSGSRETPSSILLGSNQGSDHSRSNKSSWSSSDEKRGSTRSDHNTSTSTKSLLPKESR | |
| LDTFWD | |

A search of sequence databases reveals that the NOV61 amino acid sequence has 1201 of 1201 amino acid residues (100%) identical to, and 1201 of 1201 amino acid residues (100%) similar to, the 1205 amino acid residue ptnr:SPTREMBL-ACC:Q9ULU4 protein from Homo sapiens (Human) (KIAA1125 PROTEIN). Public amino acid databases include the GenBank databases, SwissProt, PDB and PIR.

NOV61 is expressed in at least Brain, Lymphoid tissue, Kidney, Whole Organism, Bone Marrow, Prostate, Lung, Lung Pleura, Retina. This information was derived by determining the tissue sources of the sequences that were included in the invention including but not limited to SeqCalling sources, Public EST sources, Literature sources, and/or RACE sources.

The disclosed NOV61 polypeptide has homology to the amino acid sequences shown in the BLASTP data listed in Table 61C.

TABLE 61C
BLAST results for NOV61

| Gene Index/ | | Length | Identity | Positives | |
|---|---|---|---|---|---|
| Identifier | Protein/Organism | (aa) | (%) | (%) | Expect |
| gi\|6329749\|dbj\|BAA86439.1\| | KIAA1125 protein | 1205 | 1201/1201 | 1201/1201 | 0.0 |
| (AB032951) | [Homo sapiens] | | (100%) | (100%) | |
| gi\|17980969\|gb\|AAL50790.1\| | se14-3r protein | 995 | 992/1041 | 994/1041 | 0.0 |
| AF454056_1 | [Homo sapiens] | | (95%) | (95%) | |
| (AF454056) | | | | | |
| gi\|14786224\|ref\|XP_ | similar to | 949 | 932/933 | 933/933 | 0.0 |
| 012932.3\| | protein kinase C | | (99%) | (99%) | |
| (XM_012932) | binding protein 1 | | | | |
| | (H. sapiens) | | | | |
| | [Homo sapiens] | | | | |
| gi\|11385648\|gb\|AAG34905.1\| | CTCL tumor | 764 | 762/810 | 763/810 | 0.0 |
| AF273045_1 | antigen se14-3 | | (94%) | (94%) | |
| (AF273045) | [Homo sapiens] | | | | |
| gi\|13677199\|emb\|CAC19781.1\| | dJ569M23.1.1 | 521 | 520/520 | 520/520 | 0.0 |
| (AL031666) | (protein kinase C | | (100%) | (100%) | |
| | binding protein 1, | | | | |
| | isoform 1 | | | | |
| | (KIAA1125)) [Homo | | | | |
| | sapiens] | | | | |

Table 61D-F lists the domain descriptions from DOMAIN analysis results against NOV61. This indicates that the NOV61 sequence has properties similar to those of other proteins known to contain this domain.

TABLE 61D
Domain Analysis of NOV61

gnl|Smart|smart00297, BROMO, bromo domain
CD-Length = 109 residues, 94.5% aligned
Score = 81.3 bits (199), Expect = 3e−16

TABLE 61E
Domain Analysis of NOV61

gnl|Pfam|pfam00628, PHD, PHD-finger. PHD folds
into an interleaved type of Zn-finger chelating 2 Zn
ions in a similar manner to that of the RING and FYVE
domains.
CD-Length = 49 residues, 93.9% aligned
Score = 56.6 bits (135), Expect = 8e−09

TABLE 61F
Domain Analysis of NOV61

gnl|Pfam|pfam01753, zf-MYND. MYND finger.
CD-Length = 38 residues, 100.0% aligned
Score = 45.1 bits (105), Expect = 2e−05

Mukai and Ono (1994) isolated a cDNA for a protein kinase, designated PKN by them, from a human hippocampus cDNA library. The putative 942-amino acid protein has leucine zipper-like sequences at its amino terminus and contains a domain with strong similarity to that of the protein kinase C family. Ubiquitous expression in human tissues was shown. Antisera detected a 120-kD recombinantly expressed protein on Western blots. The protein showed intrinsic protein kinase activity that was abolished by a mutation in the predicted ATP binding site.

Palmer et al. (1994) used degenerate PCR to isolate 3 novel members of the closely related protein kinase C (PKC) family, termed PRK1, PRK2 (602549), and PRK3. Palmer et al. (1995) cloned a full-length cDNA of PRK1 from a human fetal brain library. Using Northern blot and RT-PCR analyses Palmer et al. (1995) detected expression of PRK1 in all tissues and cell lines tested.

In a study of proteins that bind to the rho GTPase (see Ridley and Hall, 1992), Amano et al. (1996) discovered 1 protein that had partial amino acid sequences identical to PKN. They found that rho binds directly to a polybasic region of the N-terminal regulatory domain that precedes the leucine zipper-like motif. The authors speculated that through this activity, PKN may mediate the rho-dependent signaling pathway.

Bartsch et al. (1998) used fluorescence in situ hybridization to map the PRKCL1 gene to 19p13.1-p12 and radiation hybrid mapping to localize the gene in subband 19p12. By segregation analysis, they mapped the corresponding mouse gene (Prkcl1) to chromosome 8.

The disclosed NOV nucleic acid of the invention encoding a KIAA1125-like protein includes the nucleic acid whose sequence is provided in Table 61A or a fragment thereof. The invention also includes a mutant or variant nucleic acid any of whose bases may be changed from the corresponding base shown in Table 61A while still encoding a protein that maintains its KIAA1125-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV61 protein of the invention includes the KIAA1125-like protein whose sequence is provided in Table 61B. The invention also includes a mutant or variant protein any of whose residues may be changed from the corresponding residue shown in Table 61B while still encoding a protein that maintains its KIAA1125-like activities and physiological functions, or a functional fragment thereof. In the mutant or variant protein, up to about 0 percent of the residues may be so changed.

The invention further encompasses antibodies-and antibody fragments, such as F_{ab} or (F_{ab})₂, that bind immunospecifically to any of the proteins of the invention.

The above defined information for this invention suggests that this KIAA1125-like protein (NOV61) may function as a member of a “KIAA1125 family”. Therefore, the NOV61 nucleic acids and proteins identified here may bc useful in potential therapeutic applications implicated in (but not limited to) various pathologics and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV61 nucleic acids and proteins of the invention are useful in potential therapeutic applications implicated in diseases including but not limited to various pathologies and disorders as indicated below. For example, a cDNA encoding the KIAA1125-like protein (NOV61) may be useful in gene therapy, and the KIAA1125-like protein (NOV61) may be useful when administered to a subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Aneurysm, Fibromuscular dysplasia, Stroke, Anemia, Bleeding disorders, Adrenoleukodystrophy, Congenital Adrenal Hyperplasia, Diabetes, Von Hippel-Lindau (VHL) syndrome, Pancreatitis, Hyperparathyroidism, Hypoparathyroidism, SIDS, Endometriosis, Fertility, Xerostomia, Hypercalceimia, Ulcers, Cirrhosis, Inflammatory bowel disease, Diverticular disease, Hirschsprung's disease, Crohn's Disease, Appendicitis, Hemophilia, hypercoagulation, Idiopathic thrombocytopenic purpura, autoimmume disease, allergies, immunodeficiencies, Graft vesus host, Ataxia-telangiectasia, Hemophilia, Lymphedema, Tonsilitis, Osteoporosis, Arthritis, Ankylosing spondylitis, Scoliosis, Tendinitis, Muscular dystrophy, Lesch-Nyhan syndrome, Myasthenia gravis, Dental disease and infection, Alzheimer's disease, Tuberous sclerosis, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Growth and reproductive disorders, Endocrine dysfunctions, Systemic lupus erythematosus, Asthma, Emphysema, ARDS, Pharyngitis, Laryngitis, Hearing loss, Tinnitus, Psoriasis, Actinic keratosis, Tuberous sclerosis, Acne, Hair growth, allopecia, pigmentation disorders, cystitis, incontinence, Renal artery stenosis, Interstitial nephritis, Glomerulonephritis, Polycystic kidney disease, Systemic lupus erythematosus, Renal tubular acidosis, IgA nephropathy, Vesicoureteral reflux, glaucoma, blindness, and Hypothyroidism, or other pathologies or conditions. The NOV61 nucleic acid encoding the KIAA1125-like protein of the invention, or fragments thereof, may further be useful in diagnostic applications, wherein the presence or amount of the nucleic acid or the protein are to be assessed.

NOV61 nucleic acids and polypeptides are further useful in the generation of antibodies that bind immuno-specifically to the novel NOV61 substances for use in therapeutic or diagnostic methods. These antibodies may be generated according to methods known in the art, using prediction from hydrophobicity charts, as described in the “Anti-NOVX Antibodies” section below. The disclosed NOV61 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV62

NOV62 includes two zinc finger BOP-like proteins disclosed below. The disclosed sequences have been named NOV62a and NOV62b.

NOV62a

A disclosed NOV62a nucleic acid of 1629 nucleotides (also referred to as CG57473-01) encoding a zinc finger BOP-like protein is shown in Table 62A. The start and stop codons are in bold letters.

TABLE 62A
NOV62a nucleotide sequence.

| | |
|---|---|
| TATAGTCTTGCTCTTTGGGATGCTGAAGGTGCTGAAATAGCAATGACAAGAGACTTGGCT | (SEQ ID NO:145) |
| CAGTGTTAAATAACTGCCGCGCTGGCCTGACAGTCTCTGAGATGACAATAGGGAGAATGG | |
| AGAACGTGGAGGTCTTCACCGCTGAGGGCAAAGGAAGGGGTCTGAAGGCCACCAAGGAGT | |
| TCTGGGCTGCAGATATCATCTTTGCTGAGCGGGCTTATTCCGCACTGGTTTTTGACAGCC | |
| TTGTTAATTTTGTGTGCCACACCTGCTTCAAGAGGCAGGAGAAGCTCCATCGCTGTGGGC | |
| AGTGCAAGTTTGCCCATTACTGCGACCGCACCTGCCAGAAGGATGCTTGGCTGAACCACA | |
| AGAATGAATGTTCGGCCATCAAGAGATATGGGAAGGTGCCCAATGAGAACATCAGGCTGG | |
| CGGCGCGCATCATGTGGCGGGTGGAGAGAGAAGGCACCGGGCTCACGGAGGGCTGCCTGG | |
| TGTCCGTGGACGACTTGCAGAACCACGTGGAGCACTTTGGGGAGGAGGAGCAGAAGGACC | |
| TGCGGGTGGACGTGGACACATTCTTGCAGTACTGGCCGCCGCAGAGCCAGCCGTTCAGCA | |
| TGCAGTACATCTCGCACATCTTCGGAGTGATTAACTGCAACGGTTTTACTCTCAGTGATC | |
| AGAGAGGCCTGCAGGCCGTGGGCGTAGGCATCTTCCCCAACCTGGGCCTGGTGAACCATG | |
| ACTGTTGGCCCAACTGTACTGTCATATTTAACAATGGCAATCATGAGGCAGTGAAATCCA | |
| TGTTTCATACCCAGATGAGAATTGAACTGCGGGCCCTAGGCAAGATCTCAGAAGGAGAGG | |
| AGCTGACTGTGTCCTATATCGACTTCCTCAACGTTAGTGAAGAACGCAAGAGGCAGCTGA | |
| AGAACCAGTACTACTTTGACTGCACATGTGAACACTGCCAGAAAAAACTGAAGGATGACC | |
| TCTTCCTGGGGGTGAAAGACAACCCCAAGCCCTCTCAGGAAGTGGTGAAGGAGATGATAC | |
| AATTCTCCAAGGATACATTGGAAAAGATAGACAAGGCTCGTTCCGAGGGTTTGTATCATG | |
| AGGTTGTGAAATTATGCCGGGAGTGCCTGGAGAAGCAGGAGCCAGTGTTTGCTGACACCA | |
| ACATCTACATGCTGCGGATGCTGAGCATTGTTTCGGAGGTCCTTTCCTACCTCCAGGCCT | |
| TTGAGGAGGCCTCGTTCTATGCCAGGAGGATGGTGGACGOCTATATGAAGCTCTACCACC | |
| CCAACAATGCCCAACTGGGCATGGCCGTGATGCGGGCAGGGCTGACCAACTGGCACGCTG | |
| GTAACATTGAGGTGGGGCACGGGATGATCTGCAAAGCCTATGCCATTCTCCTGGTGACAC | |
| ACGGACCCTCCCACCCCATCACTAAGGACTTAGAGGCCATGCGGGTGCAGACGGAGATGG | |
| AGCTACGCATGTTCCGCCAGAACGAATTCATGTACTACAAGATGCGCGAGGCTGCCCTGA | |
| ACAACCAGCCCATGCAGGTCATGGCCGAGCCCAGCAATGAGCCATCCCCAGCTCTGTTCC | |
| ACAAGAAGCAATGAGGACTGCCCACTGCAGGAGGGGCGATGTGGCTGGGGAGCTAGGGAG | |
| AGACTCTGG | |

In a search of public sequence databases, the NOV62a nucleic acid sequence, located on chromsome 2 has 1392 of 1573 bases (88%) identical to a gb:GENBANK-ID:MMU76373|acc:U76373.2 mRNA from Mus musculus (Mus musculus skm-BOP1 (Bop) mRNA, complete cds). Public nucleotide databases include all GenBank databases and the GeneSeq patent database.

The disclosed NOV62a polypeptide (SEQ ID NO:146) encoded by SEQ ID NO:145 has 490 amino acid residues and is presented in Table 62B using the one-letter amino acid code. Signal P, Psort and/or Hydropathy results predict that NOV62a has no signal peptide and is likely to be localized in the cytoplasm with a certainty of 0.6500.

TABLE 62B
Encoded NOV62a protein sequence.

| | |
|---|---|
| MTIGRMENVEVFTAEGKGRGLKATKEFWAADIIFAERAYSAVVFDSLVNFVCHTCFKRQE | (SEQ ID NO:146) |
| KLHRCGQCKFAHYCDRTCQKDAWLNHKNECSAIKRYGKVPNENIRLAARIMWRVEREGTG | |
| LTEGCLVSVDDLQNHVEHFGEEEQKDLRVDVDTFLQYWPPQSQPFSMQYISHIFGVINCN | |
| GFTLSDQRGLQAVGVGIFPNLGLVNHDCWPNCTVIFNNGNHEAVKSMFHTQMRIELRALG | |
| KISEGEELTVSYIDFLNVSEERKRQLKKQYYFDCTCEHCQKKLKDDLFLGVKDNPKPSQE | |
| VVKEMIQFSKDTLEKIDKARSEGLYHEVVKLCRECLEKQEPVFADTNIYMLRMLSIVSEV | |
| LSYLQAFEEASFYARRMVDGYMKLYHPNNAQLGMAVMRAGLTNWHAGNIEVGHGMICKAY | |
| AILLVTHGPSHPITKDLEAMRVQTEMELRMFRQNEFMYYKMREAALNNQPMQVMAEPSNE | |
| PSPALFHKKQ | |

A search of sequence databases reveals that the NOV62a amino acid sequence has 458 of 485 amino acid residues (94%) identical to, and 478 of 485 amino acid residues (98%) similar to, the 485 amino acid residue ptnr:SPTREMBL-ACC:P97443 protein from Mus musculus (Mouse) (ZINC-FINGER PROTEIN BOP). Public amino acid databases include the GenBank databases, SwissProt, PDB and PIR.

NOV62a is expressed in at least Whole Organism, Heart, Lung, Prostate, Skeletal Muscle. This information was derived by determining the tissue sources of the sequences that were included in the invention including but not limited to SeqCalling sources, Public EST sources, Literature sources, and/or RACE sources.

NOV62b

A disclosed NOV62b nucleic acid of 1555 nucleotides (also referred to as CG57473-01) encoding a zinc finger BOP-like protein is shown in Table 62C. The start and stop codons are in bold letters.

TABLE 62C
N0V62b nucleotide sequence.

| | |
|---|---|
| AAGGTGCTGAAATAGCAATGACAAGAGACTTAGCTCAGTGTTAAATAACTGCCGCGCTGG | (SEQ ID NO:147) |
| CCTGACAGTTTCTGAGATGACAATAGGGAGAATGCAGAACGTGGAGGTCTTCACCGCTGA | |
| GGGCAAAGGAAGGGGTCTGAAGGCCACCAAGGAGTTCTGGGCTGCAGATATCATCTTTGC | |
| TGATCGGGCTTATTCCGCAGTGGTTTTTGACAGCCTTGTTAATTTTGTGTGCCACACCTG | |
| CTTCAAGAGGCAGGAGAAGCTCCATCGCTGTGGGCAGTGCAAGTTTGCCCATTACTGCGA | |
| CCGCACCTGCCAGAAGGATGCTTGGCTGAACCACAAGAATGAATGTTCGGCCATCAAGAG | |
| ATATGGGAAGGTGCCCAATGAGAACATCAGGCTGGCGGCGCGCATCATGTGGAGGGTGGA | |
| GAGAGAAGGCACCGGGCTCACGGAGGGCTGCCTGGTGTCCGTGGACGACTTGCAGAACCA | |
| CGTGGAGCACTTTGGGGAGGAGGAGCAGAAGGACCTGCGCGTGGACGTGGACACATTCTT | |
| GCAGTACTGGCCGCCGCAGAGCCAGCAGTTCAGCATGCAGTACATCTCGCACATCTTCGG | |
| AGTGATTAACTGCAACGGTTTTACTCTCAGTGATCAGAGAGGCCTGCACGCCGTGGGCGT | |
| AGGCATCTTCCCCAACCTGGGCCTGGTGAACCATGACTGTTGGCCCAACTGTACTGTCAT | |
| ATTTAACAATGGCAATCATGAGGCAGTGAAATCCATGTTTCATACCCAGATGAGAATTGA | |
| GCTCCGGCCCCTAGGCAAGATCTCAGAAGGAGACGAGCTGACTGTGTCCTATATTGACTT | |
| CCTCAACGTTAGTGAAGAACGCAAGAGGCAGCTGAAGAAGCAGTACTACTTTGACTGCAC | |
| ATGTGAACACTGCCAGAAAAAACTGAAGGATGACCTCTTCCTGGGGGTGAAAGACAACCC | |
| CAAGCCCTCTCAGGAAGTGGTGAAGGAGATGATACAATTCTCCAAGGATACATTGGAAAA | |
| GATAGACAAGGCTCGTTCCGAGGGTTTGTATCATGAGGTTGTGAAATTATGCCGGGAGTG | |
| CCTGGAGAAGCAGGAGCCAGTGTTTGCTGACACCAACATCTACATGCTGCGGATGCTCAG | |
| CATTGTTTCGGAGGTCCTTTCCTACCTCCAGGCCTTTGAGGAGGCCTCGTTCTATGCCAG | |
| GAGGATGGTGGACGGCTATATGAAGCTCTACCACCCCAACAATGCCCAACTGGGCATGGT | |
| CGTGATGCGGGCAGGGCTGACCAACTGGCATCCTGGTAACATTGAGGTGGGGCACGGGAT | |
| GATCTGCAAAGCCTATGCCATTCTCCTGGTGACACACGGACCCTCCCACCCCATCACTAA | |
| GGACTTAGAGGCCATGCGGGTGCAGACGGAGATGGAGCTACGCATGTTCCGCCAGAACGA | |
| ATTCATGTACTACAAGATGCGCGAGGCTGCCCTGAACAACCAGCCCATGCAGGTCATGGC | |
| CGAGCCCAGCAATGAGCCATCCCCAGCTCTGTTCCACAAGAAGCAATGAGGACTG | |

In a search of public sequence databases, the NOV62b nucleic acid sequence, located on chromsome 2 has has 1356 of 1525 bases (88%) identical to a gb:GENBANK-k ID:MMU76373|acc:U76373.2 mRNA from Mus musculus (Mus musculus skin-BOP1 (Bop) mRNA, complete cds). Public nucleotide databases include all GenBank databases and the GeneSeq patent database.

The disclosed NOV62b polypeptide (SEQ ID NO:148) encoded by SEQ ID NO:147 has 490 amino acid residues and is presented in Table 62D using the one-letter amino acid code. Signal P, Psort and/or Hydropathy results predict that NOV62b has no signal peptide and is likely to be localized in the cytoplasm with a certainty of 0.6500.

TABLE 62D
Encoded NOV62b protein sequence.

| | |
|---|---|
| MTIGRMENVEVFTAEGKGRGLKATKEFWAADIIFADRAYSAVVFDSLVNFVCHTCFKRQE | (SEQ ID NO:148) |
| KLHRCGQCKFAHYCDRTCQKDAWLNHKNECSAIKRYGKVPNENIRLAARIMWRVEREGTG | |
| LTEGCLVSVDDLQNHVEHFGEEEQKDLRVDVDTFLQYWPPQSQQFSMQYISHIFGVINCN | |
| GFTLSDQRGLQAVGVGIFPNLGLVNHDCWPNCTVIFNNGNHEAVKSMFHTQMRIELRALG | |
| KISEGEELTVSYIDFLNVSEERKRQLKKQYYFDCTCEHCQKKLKDDLFLGVKDNPKPSQE | |
| VVKEMIQFSKDTLEKIDKARSEGLYHEVVKLCRECLEKQEPVFADTNIYMLRMLSIVSEV | |
| LSYLQAFEEASFYARRMVDGYMKLYHPNNAQLGMVVMRAGLTNWHAGNIEVGHGMICKAY | |
| AILLVTHGPSHPITKDLEAMRVQTEMELRMFRQNEFMYYKMREAALNNQPMQVMAEPSNE | |
| PSPALFHKKQ | |

A search of sequence databases reveals that the NOV62b amino acid sequence has 457 of 485 amino acid residues (94%) identical to, and 478 of 485 amino acid residues (98%) similar to, the 485 amino acid residue ptnr:SPTREMBL-ACC:P97443 protein from Mus musculus (Mouse) (ZINC-FINGER PROTEIN BOP). Public amino acid databases include the GenBank databases, SwissProt, PDB and PIR.

NOV62b is expressed in at least Whole Organism, Heart, Lung, Prostate, Skeletal and Muscle. This information was derived by determining the tissue sources of the sequences that were included in the invention including but not limited to SeqCalling sources, Public EST sources, Literature sources, and/or RACE sources.

The disclosed NOV62b polypeptide has homology to the amino acid sequences shown in the BLASTP data listed in Table 62E.

TABLE 62E
BLAST results for NOV62b

| Gene Index/ | | Length | Identity | Positives | |
|---|---|---|---|---|---|
| Identifier | Protein/Organism | (aa) | (%) | (%) | Expect |
| gi\|5870832\|gb\|AAC53021.2\| | skm-BOP1 [Mus | 485 | 458/485 | 478/485 | 0.0 |
| (U76373) | musculus] | | (94%) | (98%) | |
| gi\|10257425\|ref\|NP_ | CD8beta opposite | 472 | 444/485 | 465/485 | 0.0 |
| 033892.1\| | strand [Mus | | (91%) | (95%) | |
| (NM_009762) | musculus] | | | | |
| gi\|1809322\|gb\|AAC53020.1\| | t-BOP [Mus | 456 | 419/447 | 437/447 | 0.0 |
| (U76371) | musculus] | | (93%) | (97%) | |
| gi\|16930387\|gb\|AAL31880.1\| | cardiac and | 486 | 397/486 | 441/486 | 0.0 |
| AF410781_1 | skeletal muscle- | | (81%) | (90%) | |
| (AF410781) | specific BOP1 | | | | |
| | [Gallus gallus] | | | | |
| gi\|16930389\|gb\|AAL31881.1\| | cardiac and | 473 | 384/486 | 428/486 | 0.0 |
| AF410782_1 | skeletal muscle- | | (79%) | (88%) | |
| (AF410782) | specific BOP2 | | | | |
| | [Gallus gallus] | | | | |

Table 62F-G lists the domain descriptions from DOMAIN analysis results against NOV62b. This indicates that the NOV62b sequence has properties similar to those of other proteins known to contain this domain.

TABLE 62F
Domain Analysis of NOV62b

gnl|Pfam|pfam01753, zf-MYND, MYND finger.
CD-Length = 38 residues, 100.0% aligned
Score = 57.8 bits (138), Expect = 1e−09

TABLE 62G
Domain Analysis of NOV62b

gnl|Smart|smart00317, SET, SET (Su(var)3-9,
Enhancer-of-zeste. Trithorax) domain; Putative methyl
transferase, based on outlier plant homologues
CD-Length = 125 residues, 44.0% aligned
Score = 53.9 bits (128), Expect = 2e−08

Transcriptional regulatory proteins containing tandemly repeated zinc finger domains are thought to be involved in both normal and abnormal cellular proliferation and differentiation. One abundant class of such transcriptional regulators resembles the Drosophila Kruppel segmentation gene product due to the presence of repeated Cys2-His2 (C₂H2) zinc finger domains that are connected by conserved sequences, called H/C links. See ZNF91 (603971) for general information on zinc finger proteins.

By screening a human insulinoma cDNA library with a degenerate oligonucleotide corresponding to the H/C linker sequence, Tommerup et al. (1993) isolated cDNAs potentially encoding zinc finger proteins. Tommerup and Vissing (1995) performed sequence analysis on a number of these cDNAs and identified several novel zinc finger protein genes, including ZNF36, which they called ZNF139. Th1 ZNF139 cDNA predicts a protein belonging to the Kruppel family of zinc finger proteins.

By isotopic in situ hybridization, Rousseau-Merck et al. (1995) mapped the ZNF36 gene, which they called KOX18, to 7q21-q22. From pulsed field gel electrophoresis studies, they showed that KOX18 is within less than 250 kb of KOX25 (ZNF38; 601261). Rousseau-Merck et al. (1995) tabulated 18 different KOX genes that had been located in pairs within 9 DNA fragments of 200 to 580 kb on 7 different chromosomes. By FISH, Tommerup and Vissing (1995) mapped the ZNF36 gene to 7q2.13-q22.1.

The disclosed NOV62 nucleic acid of the invention encoding a zinc finger BOP-like protein includes the nucleic acid whose sequence is provided in Table 62A or 62C or a fragment thereof. The invention also includes a mutant or variant nucleic acid any of whose bases may be changed from the corresponding base shown in Table 62A or 62C while still encoding a protein that maintains its zinc finger BOP-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried Out at least in part to enhance the chemical stability of the modified nucleic acid, such that they may be used, for example, as antisense binding nucleic acids in therapeutic applications in a subject. In the mutant or variant nucleic acids, and their complements, up to about 12 percent of the bases may be so changed.

The disclosed NOV62 protein of the invention includes the zinc finger BOP-like protein whose sequence is provided in Table 62B or 62D. The invention also includes a mutant or variant protein any of whose residues may be changed from the corresponding residue shown in Table 62B or 62D while still encoding a protein that maintains its zinc finger BOP-like activities and physiological functions, or a functional fragment thereof. In the mutant or variant protein, up to about 6 percent of the residues may be so changed.

The invention further encompasses antibodies and antibody fragments, such as F_{ab} or (F_{ab})₂, that bind immunospecifically to any of the proteins of the invention. The above defined information for this invention suggests that this zinc finger B131-like protein (NOV62) may function as a member of a “zinc finger BOP family”. Therefore, the NOV62 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV62 nucleic acids and proteins of the invention are useful in potential therapeutic applications implicated in diseases including but not limited to various pathologies and disorders as indicated below. For example, a cDNA encoding the zinc finger BOP-like protein (NOV62) may be useful in gene therapy, and the zinc finger BOP-like protein (NOV62) may be useful when administered to a subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Aneurysm, Fibromuscular dysplasia, Stroke, Anemia, Bleeding disorders, Adrenoleukodystrophy, Congenital Adrenal Hyperplasia, Diabetes, Von Hippel-Lindau (VHL) syndrome, Pancreatitis, Hyperparathyroidism, Hypoparathyroidism, SIDS, Endometriosis, Fertility, Xerostomia, Hypercalceimia, Ulcers, Cirrhosis, Inflammatory bowel disease, Diverticular disease, Hirschsprung's disease, Crohn's Disease, Appendicitis, Hemophilia, hypercoagulation, Idiopathic thrombocytopenic purpura, autoimmume disease, allergies, immunodeficiencies, Graft vesus host, Ataxia-telangiectasia, Hemophilia, Lymphedema, Tonsilitis, Osteoporosis, Arthritis, Ankylosing spondylitis, Scoliosis, Tendinitis, Muscular dystrophy, Lesch-Nyhan syndrome, Myasthenia gravis, Dental disease and infection, Alzheimer's disease, Tuberous sclerosis, Parkinson's disease, Huntington's disease, Cerebral palsy, Epilepsy, Lesch-Nylan syndrome, Multiple sclerosis, Ataxia-telangiectasia, Leukodystrophies, Behavioral disorders, Addiction, Anxiety, Pail, Neuroprotection, Growth and reproductive disorders, Endocrine dysfunctions, Systemic lupus erythematosus, Asthma, Emphysema, ARDS, Pharyngitis, Laryngitis, Hearing loss, Tinnitus, Psoriasis, Actinic keratosis, Tuberous sclerosis, Acne, Hair growth, allopecia, pigmentation disorders, cystitis, incontinence, Renal artery stenosis, Interstitial nephritis, Glomerulonephritis, Polycystic kidney disease, Systemic lupus erythematosus, Renal tubular acidosis, IgA nephropathy, Vesicoureteral reflux, glaucoma, blindness, and Hypothyroidism, or other pathologies or conditions. The NOV62 nucleic acid encoding the zinc finger BOP-like protein of the invention, or fragments thereof, may further be useful in diagnostic applications, wherein the presence or amount of the nucleic acid or the protein are to be assessed.

NOV62 nucleic acids and polypeptides are further useful in the generation of antibodies that bind immuno-specifically to the novel NOV62 substances for use in therapeutic or diagnostic methods. These antibodies may be generated according to methods known in the art, using prediction from hydrophobicity charts, as described in the “Anti-NOVX Antibodies” section below. The disclosed NOV62 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV63

A disclosed NOV63 nucleic acid of 3647 nucleotides (also referred to as CG57777-01) encoding a secreted protein-like protein is shown in Table 63A. The start and stop codons are in bold letters.

TABLE 63A
NOV63 nucleotide sequence.

(SEQ ID NO:149)

GACCTACAGAGAGACTTAGACTCCTACACAATAGTAGTGGGAGACTTTAACACCCCACTGTCAACATTAG
ACAGATTGAGACAGAAAATTAACAAGGATATTCAGTACTTGAACTCAGCTCTGGACCAAGCAGACCTAAT
AGACATCTACAGAACTCTCCACTCCAAATCAACAGAATATGCATTTTTCTCAGCACTACATCACACTTAT
TCTAAAATTCACCACATAATTGGAAGTAAAACACTCCTTAGCAAATGCAAAAGAATGGAAATCATAACAA
ACAGTCTCTCAGACCACAGTGCAATCAAATTAGAACTCAGGATTAAGAAACTCACTCAAAACCACACAAC
TACATGGAAACTGAAAAACCTGCTCCTGAATAACTACTTGGTAAATAATGAAATTAAGGCAGAAATAAAT
AAGTTCTGTGAAACCAATGAGAACAAAGACACAACGTACCAGAATTTCTGGGACACAGCTAAAGCAGTGG
TTAGAGGGAAATTTATAGCACTAAATGCGCACAGGAGAAAGCAAGAAAGATGTAAAATCAACACCCTAAC
ATCACAATTAAAAGAACTAGAGAAGCAAGAGCAAACAAATTCAAAAGCTAACAGAAGACAAGAAATAACT
AAGATCATAGCAGAACTGAAGGAGATAAAGACACGAAAAACCCATCAAAAAATCAATGAATCTGGGAGCT
GGTTTTTTGAAAAGATTAACAAAATAGATAGACAACTAGCCAGACTAATAAAGAAGAGAAGAGAGAAGAA
TCAAATAGATGCAATAAAAAATGATAAAGGGGATATCACTGCTGATCCCACAGAAATACAAACTACCATC
AGAGAATACTATAAACACCTCTATGCAAATAAACTAGAAAATCTAGAAGAAATGGATAAATTCCTGGCCA
CATGCACCCTCCCAAGACTAAACCAGGAAGAGTTAGAATCCCTGAATAGACAAATAACAAGTTCTGAAAT
TAAGGCAGTAATTAATAGCCTACCAACCAAACAAAAGCCCGGACCAGATGGATTCACAGCTGAATTCTAC
CAGAGGTACAAAGAGGAGCTGGTACCATTCCTTCTGAAACTATTCCAAACAACAGAAAAAGAGGGACTCC
TTCCTAACTCATTTTATGAGGCAAGCATCATGCTGATGCCAAAATCTGGCAGAGACACAACAAAAAAAGA
AAATTTCAGGCCTATATCCCTGATGAACATCGATGTGAAAATCCTCAATAAAATACTGGCAAACCAAATC
TTGCAGCACATCAAAAAGCTTATCCACGATGATCAAGTTGGCTTCATCCCTGGGATGCAAGGCTGGTTCA
ACATATGCAAATCAATCAACATAATCCATCACATAAATAGCACCAATGACAAAAACCACATGATTATCTC
AATAGATGCAGAAAAGGCCTTTGGTAAAATTCAACACCCCTTCATGCTAAAAACTCTAAATAAGCTAGGT
ATTGATGGAACGTATCTCAAAATAATAAGAGCTGTTTATGACAAACCCACAGCCAATATCATACTGACTG
GGCAAAAGCTGGAAGCATTCCCTTTGAAAACCAGCACAAGACAAGTATGCCCTCTCTCACCACTCCTATT
CAACATGGTATTGGAAGTTCTGGCTAGGGCAATCAGGCAAGAGAAAGAAATAAAGCATATCCAAATAGGA
AGAGAGGAAGTCAAATTGTCCCTGTTTGCAGATGACATGATTGTATATTTAGAAAACCCCATCGTCTCAG
CCCAAAATCTCCTTAAGCTGATAAGAAACTTCAGCAAAGTCTCGGGATACAAAATCAATGTGCAAAAATC
ACAAGCATTCCTATACATCAATAATAGACAAACAGAGAGCCAAATCGTGAGTGAACTCCCATTCACAATT
GTTACAAAGAGAATACAATACCTAGGAATACAACTTACAAGGGATGTGAAGGACCTCTTCAAGGAGAACT
ACAAACCACTGCTCAAGGAAATAAGAGAGGACACAAACAAATGGAAAAACATTCTATGCTCATGGATAGG
AAGAATCAATATCGTGAAAATGACCATGCTGCCCAAAGTAATTTATAGATTCAACACTATGCCCATCAAG
CTACCATTGACTTTCTTCACGGAATCAGACAAAACTACTTTAAATTTCATATGGAACCAAAAAAGAGCCT
GCACAGCCAAGACAATCCTAAGCAAAAAGAACAAAGCTGGAGGCATCACACTACCTAACTTCAAACTATA
CTACAAGGCTACAGTGACCAAAACAGCATGGTACTGGTACCAAAACAGATATACAGACCAATGGAACAGA
ATAGAGGCCTCAGAAATAACACCACACATCTACAACCACCTGATCTTTGACAAACCTGACACAAACAAGC
AATGGGGAAAAGGATCTCTATTTAATAAATGGTGTTGGGAAAACTGGCTAGCCATATGCAGAAAACTGAA
ACTGGACCCCTTCCTTACACTTTATACAAAAATTAATTCAAGCTGGATTAAAGACTTAAATGTAAGACCT
AAAACAATAAAAATCCTAGAAGAAAACCTGGGCAATACCATTCAGGACATAGGCATGGGCAAAGACTTCG
TGACTGTAACACCAAAAGCAATGGCAACAAAAGCCAAAATTGACAAATGGGATCTAATTAAACTAAAGAG
CTTCTGCACAGCAAAAGAAACTGTCATCAGGGTGAACAGGCAACCTACAGAATGGGAAAAATTTTTTGCA
ATCTGTCCATCTGACAAAGGGCTAATATCCAGAATCTACAAGGAACTTAAACAAATTTACAAGAAAAAAA
CAAACAACCCTATCAAAAAGTGGGCAAAGGCTATGAACAGACACTTCTCAAAAGAAGACATTTATGCAGC
CAAAAGACATATGAAAAAATGGTCATCATCACTGGTCTTCAGGGAAATGCAAATCAAAACCACAATGAGA
TACCATCTCATGCCAGTTAGAATGGTGATCATTAGAAAGTCAGGAAACAACACATGCATGCAAATCAAAA
CCACAATGAGATACCATCTCATGCCAGTTAGAATGGTGATCATTAGAAAGTCAGGAAACAACACATGCAG
AGGATGTGGAGAAATAGGAATGCTTTTACACTGTTGGTGGGAGTGTAAACTAGTTCAACCATTGTGGAAG
ACAGTGTGGCGATTCCTCAAGGATCTAGAACCAGAAATACCATTAGACCCAGCAATCCCATTACTGGGTA
TATACCCAAATGATTATAAATCATGCTACTATAAAGACACATGCACACGTATGTTTATTGCGGCACTATT
CACAATAGCAAAGACTTGGAACCAACCCAAATGCCCATCAGTGAGAGTCATAAAGAAAATGTGGCACATA
TACATCATGGAATACTATGCAGCCATAAAAAAGGATGAGTTCATGTCCTTTGCAGGGACATGGATGAATC
TGGAAACCACCATTCTCAGCAAACTAACACAGGAACAGAAAACCAAATACCGCTTGTTCTCACTCGTAAG
TTGGAGTTGAACAATGAGAACACATGGACACAGGGAGGGGAACAACACCAGGGCCTGTCAGGGGGTAGGG
GGGATAGGGGAGGGATAGCATTAAGAGAAATACCTAATGTAGATGACGGGTTGATGGGTGCAGCAAACCA
CCAAGGC

In a search of public sequence databases, the NOV63 nucleic acid sequence, located on chromsome 13 has 3146 of 3647 bases (86%) identical to a gb:GENBANK-ID:HSIL25 FL|acc:X67285.1 mRNA from Homo sapiens (H.sapiens gene for interleukin-2 (5′ flanking region)). Public nucleotide databases include all GenBank databases and the GeneSeq patent database.

The disclosed NOV63 polypeptide (SEQ ID NO:150) encoded by SEQ ID NO:149 has 1081 amino acid residues and is presented in Table 63B using the one-letter amino acid code. Signal P, Psort and/or Hydropathy results predict that NOV63 has no signal peptide and is likely to be localized in the cytoplasm with a certainty of 0.6000.

TABLE 63B
Encoded NOV63 protein sequence.

| | |
|---|---|
| MEIITNSLSDHSAIKLELRIKKLTQNHTTTWKLKNLLLNNYLVNNEIKAEINKFCETNEN | (SEQ ID NO:150) |
| KDTTYQNFWDTAKAVVRGKFIALNAHRRKQERCKINTLTSQLKELEKQEQTNSKANRRQE | |
| ITKIIAELKEIKTRKTHQKINESGSWFFEKINKIDRQLARLIKKRREKNQIDAIKNDKGD | |
| ITADPTEIQTTIREYYKHLYANKLENLEEMDKFLATCTLPRLNQEELESLNRQITSSEIK | |
| AVINSLPTKQKPGPDGFTAEFYQRYKEELVPFLLKLFQTTEKEGLLPNSFYEASIMLMPK | |
| SGRDTTKKENFRPISLMNIDVKILNKILANQILQHIKKLIHDDQVGFIPGMQGWFNICKS | |
| INIIHHINSTNDKNHMIISIDAEKAFGKIQHPFMLKTLNKLGIDGTYLKIIRAVYDKPTA | |
| NIILTGQKLEAFPLKTSTRQVCPLSPLLFNNVLEVLARAIRQEKEIKHIQIGREEVKLSL | |
| FADDMIVYLENPIVSAQNLLKLIRNFSKVSGYKINVQKSQAFLYINNRQTESQIVSELPF | |
| TIVTKRIQYLGIQLTRDVKDLFKENYKPLLKEIREDTNKWKNILCSWIGRINIVKMTMLP | |
| KVIYRFNTMPIKLPLTFFTESDKTTLNFIWNQKRACTAKTILSKKNKAGGITLPNFKLYY | |
| KATVTKTAWYWYQNRYTDQWNRIEASEITPHIYNHLIFDKPDTNKQWGKGSLFNKWCWEN | |
| WLAICRKLKLDPFLTLYTKINSSWIKDLNVRPKTIKILEENLGNTIQDIGMGKDFVTVTP | |
| KAMATKAKIDKWKLIKLKSFCTAKETVIRVNRQPTEWEKFFAICPSDKGLISRIYKELKQ | |
| IYKKKTNNPIKKWAKAMNRHFSKEDIYAAKRHMKKWSSSLVFREMQIKTTMRYHLMPVRM | |
| VIIRKSGNNTCMQIKTTMRYHLMPVRMVIIRKSGNNTCRGCGEIGMLLHCWWECKLVQPL | |
| WKTVWRFLKDLEPEIPLDPAIPLLGIYPNDYKSCYYKDTCTRMFIAALFTIAKTWNQPKC | |
| PSVRVIKKMWHIYIMEYYAAIKKDEFMSFAGTWMNLETTILSKLTQEQKTKYRLFSLVSW | |
| S | |

A search of sequence databases reveals that the NOV63 amino acid sequence has 752 of 865 amino acid residues (86%) identical to, and 787 of 865 amino acid residues (90%) similar to, the 1010 amino acid residue patp:B38012 Human secreted protein encoded by gene 3 clone HNHCT15. Public amino acid databases include the GenBank databases, SwissProt, PDB and PIR.

The disclosed NOV63 polypeptide has homology to the amino acid sequences shown in the BLASTP data listed in Table 63C.

TABLE 63C
BLAST results for NOV63

| Gene Index/ | | Length | Identity | Positives | |
|---|---|---|---|---|---|
| Identifier | Protein/Organism | (aa) | (%) | (%) | Expect |
| gi\|106322\|pir\|\|B34087 | protein (L1H 3′ | 1280 | 919/1078 | 966/1078 | 0.0 |
| | region) - human | | (85%) | (89%) | |
| gi\|2072948\|gb\|AAC51261.1\| | putative p150 | 1275 | 904/1078 | 959/1078 | 0.0 |
| (U93563) | [Homo sapiens] | | (83%) | (88%) | |
| gi\|339777\|gb\|AAB59368.1\| | ORF2 contains a | 1275 | 898/1078 | 957/1078 | 0.0 |
| (M80344) | reverse | | (83%) | (88%) | |
| | transcriptase | | | | |
| | domain. [Homo | | | | |
| | sapiens] | | | | |
| gi\|5052951\|gb\|AAD38785.1\| | unknown [Homo | 1275 | 900/1078 | 957/1078 | 0.0 |
| AF149422_2 | sapiens] | | (83%) | (88%) | |
| (AF149422) | | | | | |
| gi\|2136112\|pir\|\|S65824 | reverse | 1275 | 898/1078 | 957/1078 | 0.0 |
| | transcriptase | | (83%) | (88%) | |
| | homolog - human | | | | |
| | transposon L1.1 | | | | |

Table 63D lists the domain descriptions from DOMAIN analysis results against NOV63. This indicates that the NOV63 sequence has properties similar to those of other proteins known to contain this domain.

TABLE 63D
Domain Analysis of NOV63

gnl|Pfam|pfam00078, rvt, Reverse transcriptase
(RNA-dependent DNA polymerase). A reverse
transcriptase gene is usually indicative of a mobile
element such as a retrotransposon or retrovirus. Reverse
transcriptases occur in a variety of mobile elements,
including retrotransposons, retroviruses, group II
introns, bacterial msDNAs, hepadnaviruses, and
caulimoviruses.
CD-Length = 208 residues, 97.6% aligned
Score = 99.0 bits (245), Expect = 1e−21

Secreted proteins can act as cytokines, growth factors, chemotactic factors, and ligands for cell surface receptors. Secreted functions play vital roles in the regulation of cell motility, proliferation, differentiation and apoptosis.

The disclosed NOV63 nucleic acid of the invention encoding a secreted protein-like protein includes the nucleic acid whose sequence is provided in Table 63A or a fragment thereof. The invention also includes a mutant or variant nucleic acid any of whose bases may be changed from the corresponding base shown in Table 63A while still encoding a protein that maintains its secreted protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 14 percent of the bases may be so changed.

The disclosed NOV63 protein of the invention includes the secreted protein-like protein whose sequence is provided in Table 63B. The invention also includes a mutant or variant protein any of whose residues may be changed from the corresponding residue shown in Table 63B while still encoding a protein that maintains its secreted protein-like activities and physiological functions, or a functional fragment thereof. In the mutant or variant protein, up to about 14 percent of the residues may be so changed.

The invention further encompasses antibodies and antibody fragments, such as F_{ab} or (F_{ab})₂, that bind immunospecifically to any of the proteins of the invention.

The above defined information for this invention suggests that this secreted protein-like protein (NOV63) may function as a member of a “secreted protein family”. Therefore, the NOV63 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV63 nucleic acids and proteins of the invention are useful in potential therapeutic applications implicated in diseases including but not limited to various pathologies and disorders as indicated below. For example, a cDNA encoding the secreted protein-like protein (NOV63) may be useful in gene therapy, and the secreted protein-like protein (NOV63) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV63 nucleic acid encoding the secreted protein-like protein of the invention, or fragments thereof, may further be useful in diagnostic applications, wherein the presence or amount of the nucleic acid or the protein are to be assessed.

NOV63 nucleic acids and polypeptides are further useful in the generation of antibodies that bind immuno-specifically to the novel NOV63 substances for use in therapeutic or diagnostic methods. These antibodies may be generated according to methods known in the art, using prediction from hydrophobicity charts, as described in the “Anti-NOVX Antibodies” section below. The disclosed NOV63 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV64

A disclosed NOV64 nucleic acid of 3081 nucleotides (also referred to as CG57779-01) encoding a secreted protein-like protein is shown in Table 64A. The start and stop codons are in bold letters.

TABLE 64A
NOV64 nucleotide sequence.

(SEQ ID NO:151)

AAATGTAAAAGAACTGTCTCTCAGACCATGGTGCAATCAAAACTAGAACTCAGCATTAAGAAACTCACTC
AAAACTGCCCAACTACATGGAAACTGAACAACCTGCTCCTGAATGACTACTGGGTACATAACAAAATCAA
GGCAGAAATAAAGATGTTCTTTGAAACCAACAAGAACAAAGACACAACATACCAGAATCTCTGGGAGACA
TTCAAAGCAGTGTGTAGAGGGAAATTTATAGCACTAAATACCCATAAGAGAAAGCAGGAAAGATCTAAAA
TTGACACCCTAACATCACAATTAAAACAACTACAGAAGCAAGAGCAAACACATTCAAAAGCTAGCAGAAG
GCAAGAAATAACTAAGATCAGAGCAGAACTGAAGGAGATAGAGACACAAAAAAATGCTTCAAAAAAAAAT
GAACTTAAAAAAGATAAAGGGGTTTGGTCCACCGATCCCAGAGAAAAACACACTACCATCAGAGAATACT
ATAAACACCTGTATGCAAATAAACTAGAAAATCTGGAAGAAATGGATAAATTTCTGGACAAATACACCTT
CCCAAGACTAAACCAGGAAGAAGTTGAATCCCTGAATAGACCAATAACAGGCTCGGAAATTGAGGCAATA
ATTAATAGCTTACCAACCAAAAAAAGTCCAGGGTCAGATGGATTCACAGCCGAATTCTACCAGAGGTACA
CCCAAGACTAAACCAGGAAGAAGTTGAATCCCTGAATAGACCAATAACAGGCTCGGAAATTGAGGCAATA
TCAAAAAGTTTATCCACCACGATCAAGTGGGCTTCATCCCTAGGATGCAAGGCTGGTTTAACATATGCAA
ATCAATAAACGTAATCCAGCATATAAATAGAACCAAAGACAAAAACCACATGATTATCTCAATAGATGCA
GAAAAGGCCTTTGACAAAATTCAACAGCCCTTCATGCTAAAAACTCTCAGTAAATTAGGTATTGATATGA
CATATCTCAAAATAATAAGAGCTATCTATGACAAACCCACAGCCAATATCATACTGAATGGGCAAAAACT
GGAAGCATTCCCTTTGAAAACTGGCACAAGACATGGGTGCCCTCTCTCACCACTCCTATTCAACATAGTG
TTGGAAGTCCTGGCCAGGGCAATCAGGCAGGAGAAGGAAATAAAGGGTATTCAATTAGGAAAAGAGGAAG
TCAAATTGTCCCTGTTTGCAGATGACATGATTTTATATCTAGAAAACCCCATCGTCTCAGCCCAAAATCT
CCTTAAGCTGATAAGCAACTTCAGCAAAGTCCCAGGATACAAAATCAATGTGCAAAAATCACAAGCATTC
TTATACACCAATAACAGACAGACAGAGAGCCAAATCATGAGTGAACTCCCATTTACAATTGCTTCAAAGA
GAATAAAATACCTAGGAATCCAACTTACAAGGGATGTGAAGGACTCTTCAAGGAGAACTACAAACCCATG
CTCAATTGAAATAAAAGAGGATACAAACAAATGGAAGAACATTCCATGCTCATGGGTAAGAAGAATCAAT
ATTGTGAAAATGGCCATTCTGCCCAAGGTAATTTATAGGTTCAATGCCATCCCCATCAAGCTACCAATGG
CTTTCTTCACAGAATTGGAAAAAACTACTTTAAAGTTCATATGGAACCAAAAAAGAGCCTGCATTGCTAA
GCCTGCATTGCTAAGCCAAAAGAACAAAGCTGGAGGCATCATGCTACCTGACTTCAAACTATACTACAAG
GCCACAGTAACCAAAACAGCATGGTACTGGTACCAAAACAGATATATAGACCAATGGAACAAAGCAGAGC
CCTCAGAAATAATGCCACACATCTATAACTATCTGATCTTTGACAAACCTGACAAAAACAAGAAATCGGG
AAAGGATTCCGTATTTAATAAACGGTCCTGGGAAAACTGGCTAGCCATATGTAGAAAGCTGAAACTGGAC
CCCTTCCTTACACCTCATACAAAAATTAATTCAAGATGGATTAAAGACTTAAATGTTAGACCTAAAACCA
TAAAAACCCTAGAAGAAAACCTAGGCAATACCATTCAGGACATAGGCATGGGCAAGGACTTCATGTCTAA
AACACCAAAAGCAATGGCAACAAAAGACAAAATTGACAAATGGGATCTAATTAAACTAAAGAGCTTCTGC
ACAGCAATAGAAACTACCATCAGAGTGAACAGGCAACCTACAGAATGGGAGAAAATTTTTGCAACCTACT
CATCTGACAAAGGGCTAATATCCAGAATCCACAATGAACTCAAACAAATTTACAAGAAAAAATCAAACAA
TCACACCAGTTAGAATGGCGATCATTAAAAAGTCAGGAAACAACAGGTGCTGGAGAGGATGTGGAGAAAT
AGGAACACTTTTACGCTGTTGGTGGGACTGTAAACTAGTTCAACCATTGTGGAAGTCAGTGTGGCGATTC
TTGGAACCAACCCAAATGTCCAACAATGATAGACTGGATTAAGAAAATGTGGCACATATACACCATGGAA
TACTATGCAGCCACAAAAAAAAAGGATGAGTTCATGTCCTTTGCAGGGACATGGATGAAGCTGGAAACCA
TCATTCTCAGCAAACTATCACAAGGACAGAAAACCAAACACTGCATGTTCTCACTCATAGGTGGGAATTA
G

In a search of public sequence databases, the NOV64 nucleic acid sequence, located on chromsome 13 has 2741 of 3050 bases (89%) identical to a gb:GENBANK-ID:HSU157D4|acc:Z68871.1 mRNA from Homo sapiens (Human DNA sequence from cosmid U157D4, between markers DXS366 and DXS87 on chromosome X). Public nucleotide databases include all GenBank databases and the GeneSeq patent database.

The disclosed NOV64 polypeptide (SEQ ID NO:152) encoded by SEQ ID NO:151 has 1017 amino acid residues and is presented in Table 64B using the one-letter amino acid code. Signal P, Psort and/or Hydropathy results predict that NOV64 has no signal peptide and is likely to be localized in the cytoplasm with a certainty of 0.3906.

TABLE 64B
Encoded NOV64 protein sequence.

| | |
|---|---|
| MVQSKLELSIKKLTQNCPTTWKLNNLLLNDYWVHNKIKAEIKMFFETNKNKDTTYQNLWD | (SEQ ID NO:152) |
| TFKAVCRGKFIALNTHKRKQERSKIDTLTSQLKQLQKQEQTHSKASRRQEITKIRAELKE | |
| IETQKNASKKNELKKDKGVWSTDPREKHTTIREYYKHLYANKLENLEEMDKFLDKYTFPR | |
| LNQEEVESLNRPITGSEIEAIINSLPTKKSPGSDGFTAEFYQRYKEELVPFLLKLFQSIE | |
| KEGILPNSFDEASIILIPKPSRDTTKKENFRPISLMNIDAKILNKILAKRIQQHIKKFIH | |
| HDQVGFIPRMQGWFNICKSINVIQHINRTKDKNHMIISIDAEKAFDKIQQPFMLKTLSKL | |
| GIDMTYLKIIRAIYDKPTANIILNGQKLEAFPLKTGTRHGCPLSPLLFNIVLEVLARAIR | |
| QEKEIKGIQLGKEEVKLSLFADDMILYLENPIVSAQNLLKLISNFSKVPGYKINVQKSQA | |
| FLYTNNRQTESQIMSELPFTIASKRIKYLGIQLTRDVKDSSRRTTNPCSIEIKEDTNKWK | |
| NIPCSWVRRINIVKMAILPKVIYRFNAIPIKLPMAFFTELEKTTLKFIWNQKRACIAKPA | |
| LLSQKNKAGGIMLPDFKLYYKATVTKTAWYWYQNRYIDQWNKAEPSEIMPHIYNYLIFDK | |
| PDKNKKSGKDSVFNKRSWENWLAICRKLKLDPFLTPHTKINSRWIKDLNVRPKTIKTLEE | |
| NLGNTIQDIGMGKDFMSKTPKAMATKDKIDKWDLIKLKSFCTAIETTIRVNRQPTEWEKI | |
| FATYSSDKGLISRIHNELKQIYKKKSNNPIKKWAKDMNRHFSKEDIYAAKRHMKKCSSSL | |
| AIREMQMKTTMRYHLTPVRMAIIKKSGNNRCWRGCGEIGTLLRCWWDCKLVQPLWKSVWR | |
| FLRDLELEIPFDPAIPLLGIYPKDYKSCCYKDTCSRMFVAALFTTAKTWNQPKCPTMIDW | |
| IKKMWHIYTMEYYAATKKKDEFMSFAGTWMKLETIILSKLSQGQKTKHCMFSLIGGN | |

A search of sequence databases reveals that the NOV64 amino acid sequence has 829 of 977 amino acid residues (84%) identical to, and 864 of 977 amino acid residues (88%) similar to, the 1010 amino acid residue patp:B38012 Human secreted protein encoded by gene 3 clone HNHCT15. Public amino acid databases include the GenBank databases, SwissProt, PDB and PIR.

The disclosed NOV64 polypeptide has homology to the amino acid sequences shown in the BLASTP data listed in Table 64C.

TABLE 64C
BLAST results for NOV64

| Gene Index/ | | Length | Identity | Positives | |
|---|---|---|---|---|---|
| Identifier | Protein/Organism | (aa) | (%) | (%) | Expect |
| gi\|106322\|pir\|\|B34087 | protein (L1H 3′ | 1280 | 929/1044 | 956/1044 | 0.0 |
| | region) - human | | (88%) | (90%) | |
| gi\|5052951\|gb\|AAD38785.1\| | unknown [Homo | 1275 | 920/1044 | 949/1044 | 0.0 |
| AF149422_2 | sapiens] | | (88%) | (90%) | |
| (AF149422) | | | | | |
| gi\|2072948\|gb\|AAC51261.1\| | putative p150 | 1275 | 919/1044 | 946/1044 | 0.0 |
| (U93563) | [Homo sapiens] | | (88%) | (90%) | |
| gi\|2072958\|gb\|AAC51267.1\| | p150 [Homo | 1275 | 918/1044 | 947/1044 | 0.0 |
| (U93567) | sapiens] | | (87%) | (89%), | |
| gi\|5070622\|gb\|AAD39215.1\| | unknown [Homo | 1275 | 918/1044 | 949/1044 | 0.0 |
| AF148856_2 | sapiens] | | (87%) | (89%), | |
| (AF148856) | | | | | |

Table 64D lists the domain descriptions from DOMAIN analysis results against NOV64. This indicates that the NOV64 sequence has properties similar to those of other proteins known to contain this domain.

TABLE 64D
Domain Analysis of NOV64

gnl|Pfam|pfam00078, rvt, Reverse transcriptase (RNA-dependent
DNA polymerase). A reverse transcriptase gene is usually
indicative of a mobile element such as a retrotransposon
or retrovirus. Reverse transcriptases occur in a variety of
mobile elements, including retrotransposons, retroviruses,
group II introns, bacterial msDNAs, hepadnaviruses, and
caulimoviruses.
CD-Length = 208 residues, 97.6% aligned
Score = 109 bits (272), Expect = 9e−25

The disclosed NOV64 nucleic acid of the invention encoding a secreted protein-like protein includes the nucleic acid whose sequence is provided in Table 64A or a fragment thereof. The invention also includes a mutant or variant nucleic acid any of whose bases may be changed from the corresponding base shown in Table 64A while still encoding a protein that maintains its secreted protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 11 percent of the bases may be so changed.

The disclosed NOV64 protein of the invention includes the secreted protein-like protein whose sequence is provided in Table 64B. The invention also includes a mutant or variant protein any of whose residues may be changed from the corresponding residue shown in Table 64B while still encoding a protein that maintains its secreted protein-like activities and physiological functions, or a functional fragment thereof. In the mutant or variant protein, up to about 16 percent of the residues may be so changed.

The invention further encompasses antibodies and antibody fragments, such as F_{ab} or (F_{ab})₂, that bind immunospecifically to any of the proteins of the invention.

The above defined information for this invention suggests that this secreted protein-like protein (NOV64) may function as a member of a “secreted protein family”. Therefore, the NOV64 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV64 nucleic acids and proteins of the invention are useful in potential therapeutic applications implicated in diseases including but not limited to various pathologies and disorders as indicated below. For example, a cDNA encoding the secreted protein-like protein (NOV64) may be useful in gene therapy, and the secreted protein-like protein (NOV64) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV64 nucleic acid encoding the secreted protein-like protein of the invention, or fragments thereof, may further be useful in diagnostic applications, wherein the presence or amount of the nucleic acid or the protein are to be assessed.

NOV64 nucleic acids and polypeptides are further useful in the generation of antibodies that bind immuno-specifically to the novel NOV64 substances for use II therapeutic or diagnostic methods. These antibodies may be generated according to methods known in the art, using prediction from hydrophobicity charts, as described in the “Anti-NOVX Antibodies” section below. The disclosed NOV64 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV65

A disclosed NOV65 nucleic acid of 3021 nucleotides (also referred to as CG57781-01) encoding a secreted protein-like protein is shown in Table 65A. The start and stop codons are in bold letters.

TABLE 65A
NOV65 nucleotide sequence.

(SEQ ID NO:153)

AATGACTACTGAGTAAATAATGAAATGAAGGCAGAAATAAAGATGTTCTTTGAAACCAATGAGAACAAAG
ACACAATGTACCAGAATCTCTGGGACACATTTAAAGCAGTGTGTAGAGGGAAATTTATAGCACTAAATGC
CCACAAGAGAAAGCAGGAAAGATCTAAAATCAACATCCTAACATCACAGTTAAAAGAACTAGGGAAGCAA
GAACAAACAAATTCAAAAGCTAGCAGAAGGCAAAAAATAACTAAGATCAGAGCAGAACTGAAGGAGATAG
AGACACAAAAAACCCTTCAAAAAATCAATGAATCCAGGAGCTGGTTTTTTGAAAAGATCAACAAAATTGA
TAGACAACTAGCAAGACCAATAAAGAAGAAAAGAGAGAAGAATCAAATAGATGCAACAAAAAATGATAAA
GGGGATATCACCACTGATCCCACAGAAATACAAACTACCATCAGAGAATACTATCAACACTTCTATGCAA
ATATACTAGAAAATCTAGAAGAAATGGATAAATTCCTGGACACATACACTCTCCCAAGACTAAACCAGGA
AGAAGTTGAATCTCTGTATAGACCAATAACAGGTTCTGAAATTGAGGCAATAATTAATAGGCTACCAACC
AAAAAAAGTCCAGGACCAGATGGATTCACAGCTGAATTCTACCAGAGGTACAAAGAGGAGCTGGTACCAT
TCCTTCTGAAACTATTTCAGACAACAGAAAAAGAGGGACTCCTCCCTAACTCATTTTATGAGGCCAGCAT
CATCCTGACACCAAAACCTGGTAGAGACACAACAAAAAAAGAGAATTTTATGCCAATATCCCTGATGAAC
ATTGATGCGAAAGTCCTCAATAAAATACTGGCAAAAGCTTATCCACCACATCAAAAGCTTATCCACCACG
GTCAACTTGGCTTCATCCCTGGGATGCAAGGCTGGTTCAACATATGCAAATCAATAAATGTAGTTCATCA
CATAAACAGAACCAATGACAAAAACCACATGATTATCTCAATAGATGCAGAAAAGGCCTTCGACAATATT
CAACACCACTTCATGCTAAAAACTCTGAGTAAACTAGGTATCGATGGAACATATCTGAAAATAATAAGAG
CTATTTATGACAAACCCACAGCCAATATCATAGTGAATGGGCAAAAACTGGAAGCATTCCCTTTGAAAAC
TGGCACAAGACAAGGATGCCCTCTCTCACCACTCCTATTCAACATAGTGTTGGAAGTTCTGGCTAGGGCA
ATCAGGCAAGAGAAAGAAATAAACGGTATTCAATTAGGAAAAGAGGAAGTCAAATTGTCTCTGTGTGCAG
ATGACATGATTGTATATTTAGAAAACCCCATCGTCTCAGCCCAAAATCTCCTTAAGCTGATAAGCAACTT
CAGCAAAGTCTCAGGATACAAAATCAATGTGCAAAAATCACAAGCATTCCTATACATCAATAATAGACAA
ACAGAGAGCCAAATCATGAGTGAACTCCCATTCCCAATTACCACAAAGAGAATTAAATACCTAGGAATCC
AACTTACAAGGGATGTGAAAGACCTCTTCAAGGAGAACTACAAACCACTGCTCGAAATAAAAGAGGACAC
AAACAAATGGAAAAACATTCCATGCTCATGGATAGGAAGAATCAATATTGTGAAAATGGTCATACTGCCC
AAAGTAATTTATAGATTCAATGCTATCCCCATCAAGCTACCACTGACTTTCTTCACAGAATTGGAAAAAA
CTATTTTAAAGTTCATATGGAACCAAAAAAGAACCCAGATTGCCAAGACAATCCTAAGCAAAAAGAACAA
AGCTGGAGGCATCACACTACCTGACTTCAAACTATACTACAAGGCTACAGTAAACAAAACAGCATGGTAC
TGGTACCAAAACAGATATATAGACCAATGGAACAGAATGGAGGCCTCAGAAATAACACCACACATCTACA
ACCATCTGATCTTTGACAAACCTGACAAAAACAGGCAATGGGGAAAGGATTCTCTATTTAATAAATGGTG
ATACCATTCAGGACATAGGCATGGGCAAAGTCTTCATGACTAAAACACCAAAAGCAATGGCAACAAAAGT
CAAAATTGACAAATGGGATCTAATTAAACTAAAGAGCTTCTGCACAGCAAAAGAAACTATCATCAGAGTG
AACAGGCAACCTACAGAATGGGAGAAAATCTTTGCAACCTACCCATCTGACAAAGGGCTAATATCCAGAA
TCTACAAAGAACTCAAACAAATTTACAAGAAAAAAAAAACAACCCCATCAAAAAGTGGGCAAATACAAGA
AAAAAAAAACAACCCCATCAAAAAGTGGGCAAAGGATATGAGCAGACACTTCTCAAAAGAAGACATTTAT
GCAGCCAACAGAATGAAAAAGTGGTCATCATCACTGGTCTTCAGGGAAATGCAAATCAAAACCACAATGA
GATACCATCTCATGCCAGTTAGAATGGTGATCATTAAAAAGTCAGGAAACAACACATGCCTGAGAGGATG
TGGAGAAATAGGAATGCTTTTACACTGTTGGTGGGAGTGTAAACTAGTTCAACCATTGTGGAAGACAGTG
CAAACAATTATAAATCATGCTACTATAAAGACACACGCACACGTATGTTTATTGTGGCACTATTCACGAT
AGCAAAGAAGGATGAGTTCATGTCCTTTGCAGGGACATGGATGAAGCTGGAAACCATCATTCTAAGCAAA
CTATCACAAGGACAGAAAACCAAACACCACATGTTCTCACTCATAGGTGGGAGTTGAACAACGAGAACAC
ATGGACACAGG

In a search of public sequence databases, the NOV65 nucleic acid sequence, located on chromsome 13 has 2524 of 2878 bases (87%) identical to a gb:GENBANK-ID:F229117S02|acc:AF229118.1 mRNA from Homo sapiens (Homo sapiens acetylcholinesterase collagen-like tail subunit (COLQ) gene, exons 1A, 2, 3, 4, and 5). Public nucleotide databases include all GenBank databases and the GeneSeq patent database.

The disclosed NOV65 polypeptide (SEQ ID NO:154) encoded by SEQ ID NO:153 has 990 amino acid residues and is presented in Table 65B using the one-letter amino acid code. Signal P, Psort and/or Hydropathy results predict that NOV65 has no signal peptide and is likely to be localized in the cytoplasm with a certainty of 0.7000.

TABLE 65B
Encoded NOV65 protein sequence.

| | |
|---|---|
| MKAEIKMFFETNENKDTMYQNLWDTFKAVCRFKFIALNAHKRKQERSKINILTSQLKELG | (SEQ ID NO:154) |
| KQEQTNSKASRRQKITKIRAELKEIETQKTLQKINESRSWFFEKINKIDRQLARPIKKKR | |
| EKNQIDATKNDKGDITTDPTEIQTTIREYYQHFYANILENLEEMDKFLDTYTLPRLNQEE | |
| VESLYRPITGSEIEAIINRLPTKKSPGPDGFTAEFYQRYKEELVPFLLKLFQTTEKEGLL | |
| PNSFYEASIILTPKPGRDTTKKENFMPISLMNIDATVLNKILAKAYPPHQKLIHHGQLGF | |
| IPGMQGWFNICKSINVVHHINRTNDKNHMIISIDAEKAFDNIQHHFMLKTLSKLGIDGTY | |
| LKIIRAIYDKPTANIIVNGQKLEAFPLKTGTRQGCPLSPLLFNIVLEVLARAIRQEKEIN | |
| GIQLGKEEVKLSLCADDMIVYLENPIVSAQNLLKLISNFSKVSGYKINVQKSQAFLYINN | |
| RQTESQIMSELPFPITTKRIKYLGIQLTRDVKDLFKENYKPLLEIKEDTNKWKNIPCSWI | |
| GRINIVKMVILPKVIYRFNAIPIKLPLTFFTELEKTILKFIWNQKRTQIAKTILSKKNKA | |
| GGITLPDFKLYYKATVNKTAWYWYQNRYIDQWNRMEASEITPHIYNHLIFDKPDKNRQWG | |
| KDSLFNKWCWENWLAICRKLKLDPFLTPYTKINTRWIKDLNVRPNTIKTLEENLGNTIQD | |
| IGMGKVFMTKTPKAMATKVKIDKWDLIKLKSFCTAKETIIRVNRQPTEWEKIFATYPSKD | |
| GLISRIYKELKQIYKKKKTTPSKSGQIQEKKNNPIKKWAKDMSRHFSKEDIYAANRMKKW | |
| TVWRFLKDLEPEIPLDPAIPLLGIYPNNYKSCYYKDTRTRMFIVALFTIAKKDEFMSFAG | |
| TWMKLETIILSKLSQGQKTKHHMFSLIGGS | |

A search of sequence databases reveals that the NOV65 amino acid sequence has have 842 of 951 amino acid residues (88%) identical to, and 867 of 951 amino acid residues (91%) similar to, the 1010 amino acid residue patp:B38012 Human secreted protein encoded by gene 3 clone HNHCT15. Public amino acid databases include the GenBank databases, SwissProt, PDB and PIR.

The disclosed NOV65 polypeptide has homology to the amino acid sequences shown in the BLASTP data listed in Table 65C.

TABLE 65C
BLAST results for NOV65

| Gene Index/ | | Length | Identity | Positives | |
|---|---|---|---|---|---|
| Identifier | Protein/Organism | (aa) | (%) | (%) | Expect |
| gi\|106322\|pir\|\|B34087 | protein (L1H 3 ′ | 1280 | 885/1023 | 911/1023 | 0.0 |
| | region) - human | | (86%) | (88%) | |
| gi\|17980447\|gb\|AAL50637.1\| | unknown [Homo | 1275 | 870/1023 | 904/1023 | 0.0 |
| (AF421375) | sapiens] | | (85%) | (88%) | |
| gi\|2072948\|gb\|AAC51261.1\| | p150 [Homo | 1275 | 870/1023 | 903/1023 | 0.0 |
| (U93563) | sapiens] | | (85%) | (88%) | |
| gi\|2136112\|pir\|\|S65824 | reverse | 1275 | 868/1023 | 904/1023 | 0.0 |
| | transcriptase | | (84%) | (87%) | |
| | homolog - human | | | | |
| | transposon L1.1 | | | | |
| gi\|5052951\|gb\|AAD38785.1\| | unknown | 1275 | 868/1023 | 904/1023 | 0.0 |
| AF149422_2 | [Homo sapiens] | | (84%) | (87%) | |
| (AF149422) | | | | | |

Table 65D lists the domain descriptions from DOMAIN analysis results against NOV65. This indicates that the NOV65 sequence has properties similar to those of other proteins known to contain this domain.

TABLE 65D
Domain Analysis of NOV65

gnl|Pfam|pfam00078, rvt, Reverse transcriptase (RNA-dependent
DNA polymerase). A reverse transcriptase gene is usually
indicative of a mobile element such as a retrotransposon
or retrovirus. Reverse transcriptases occur in a variety of
mobile elements, including retrotransposons, retroviruses,
group II introns, bacterial msDNAs, hepadnaviruses, and
caulimoviruses.
CD-Length = 208 residues, 97.6% aligned
Score = 108 bits (269), Expect = 2e−24

The disclosed NOV65 nucleic acid of the invention encoding a secreted protein-like protein includes the nucleic acid whose sequence is provided in Table 65A or a fragment thereof. The invention also includes a mutant or variant nucleic acid any of whose bases may be changed from the corresponding base shown in Table 65A while still encoding a protein that maintains its secreted protein-like activities and physiological functions, or a fragment of Such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 13 percent of the bases may be so changed.

The disclosed NOV65 protein of the invention includes the secreted protein-like protein whose sequence is provided in Table 65B. The invention also includes a mutant or variant protein any of whose residues may be changed from the corresponding residue shown in Table 65B while still encoding a protein that maintains its secreted protein-like activities and physiological functions, or a functional fragment thereof. In the mutant or variant protein, up to about 12 percent of the residues may be so changed.

The invention further encompasses antibodies and antibody fragments, such as F_{ab} or (F_{ab})₂, that bind immunospecifically to any of the proteins of the invention.

The above defined information for this invention suggests that this secreted protein-like protein (NOV65) may function as a member of a “secreted protein family”. Therefore, the NOV65 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV65 nucleic acids and proteins of the invention are useful in potential therapeutic applications implicated in diseases including but not limited to various pathologies and disorders as indicated below. For example, a cDNA encoding the secreted protein-like protein (NOV65) may be useful in gene therapy, and the secreted protein-like protein (NOV65) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV65 nucleic acid encoding the secreted protein-like protein of the invention, or fragments thereof, may further be useful in diagnostic applications, wherein the presence or amount of the nucleic acid or the protein are to be assessed.

NOV65 nucleic acids and polypeptides are further useful in the generation of antibodies that bind immuno-specifically to the novel NOV65 substances for use in therapeutic or diagnostic methods. These antibodies may be generated according to methods known in the art, using prediction from hydrophobicity charts, as described in the “Anti-NOVX Antibodies” section below. The disclosed NOV65 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV66

A disclosed NOV66 nucleic acid of 3120 nucleotides (also referred to as CG57783-01) encoding a secreted protein-like protein is shown in Table 66A. The start and stop codons are in bold letters.

TABLE 66A
NOV66 nucleotide sequence.

(SEQ ID NO:155)

CTGAATGACTACTGGGTACATAACAAAATGAAGACAGAAATAAAGATGTTCTTTGAAACCAATGAGAACA
AAGACACAACATACCAGAATCTCTGGGACACATTCAAAGCAGTGTGTAGAGGGAAATTTACAGCACTAAA
TGCCCATAAGAGAAAGCAGGAAAGATCCAAAATTGACACCCTAACATCACAATTAAAACAACTACAGAAG
CAAGAGCAAACACTTTCAAAAGCTAGCAGAAGGCAAGAAATAACTAAGATCAGAGCAGAACTGAAGGAGA
TAGAGACACAAAAAACCCTTCAAAAAATCAATGAATCCAGGAGCTGGTTTTTTGAAAAGATCAACAAAAT
TGATACACTGCTAGCAAGACTAATAAAGAAGAAAAGAGAGAAGAATCAAATAGACGCAATAAAAAATGAT
AAAGCAGATATCACCACTGATCCCACAGAAATACAAACTACCATCAGAGAATACTATAAACACCTCTATG
CAAATAAACTAGAAAATCTAGAAGAAATGGATAAATTCCTTGACACATACACCCTCCCAAGAATAAACCA
GGAAGAAGTTGAATCTCTGAATAGACCAATAACAGGCTCTGAAATTGAGGCAATAATTAATAGCTTACCA
ACCAAAAAAAGTCCAGGACCAGACGGATTCACAGCCGAATTCTACCAGAAGTACAAGGAGGAGCTGATAC
CATTCCTTCTGAAACTATTCCAATCAATAGAAAAAGAGGGAATCCTCCCTAACTCATTTGATGAGGCCAG
CATCATCCTGATACCAAAGCCTGGCAGAGACACAACAAAAAAAGAGAATTTTAGACCAATATCTCTGATG
AACATTGATGCAAAAATCCTCAATAAAATACTGGCAAACCGAATCAAGCAACACATCAAAAAGCTTATCC
ACCATGATCAAGTGGGCTTCATCCCTGGGATGCAAGGCTGGTTCAACATATGCAAATCAATAAACGTAAT
CCAGCATATAAACAGAACCAAAGACAAAAACCACATGATTATCTCAATAGATGCAGAAAAGGCCTTTGAC
AAAATTCAACAGCACTTCATGCTAAAAACTCTCACCACTCCTATTCAACATAGTGTTGGAAGTCCTGGCC
TAAGAGCTATCTGTGACAAACCCACTGCCAATATCATACTGAATGGGCAAAAACTGGAAGCGTTCCCTTT
GAAAACTGGCACAAGACAAGGGTGCCCTCTCTCACCACTCCTATTCAACATAGTGTTGGAAGTCCTGGCC
AGGGCAATCAGGCAGGAGAAGGAAATAAAGGGTATTCAGTTAGGAAAAGAGGAAGTCAAATTGTCTCTGT
TTGCAGATGACATGATTGTATATCTAGAAAACCCCATCATCTCAGCCCAAAATCTCCTTAAGCTGATAAG
CAACTTCAGCAAAGTCTCAGGATACAAAATCGATGTGCAAAAATCACAAGCATTCTTATACACCAATACA
GACCAGACAGAGAGCCAAATCATGAGTGACCTCCCATTCACAATTGCTTCAAAGAGAATAAAATACCTAG
GAATCCAACTTACAAGGGATGTGAAGGACCTCTTCAAGGAGAACTACAAACCACTGCTCAATGAAATAAA
AAAGGATACAAACAAATGGAAGAACATTCCAGGCTCATGGATAGGAAGAATCAATATCGTGAAAATGGCC
ATAGAGCCCAAGGTAATTTATAGATTCAATGCCATCCCCATCAAGCTACCAATGACTTTCTTCACAGAAC
TGGAGAAAACTACTTTAAAGTTCATATGGAACCAAAAGAGAGCCCACATTGCCAAGTCAATCCTAAACCA
AAAGAACAAAGCTGGAGGCATCACACCACCTGACTTCAAACTATACTACAAGGCTACAGTAAACAAAACA
GCATGGTACTGGTACCAAAACAGAGATATAGACCAGTGGAACAGAACAGATCCCTCAGAAATAATGCCAC
ACATCTACAACTATCTGATCTTTGACAAACCTGACAAAAAGAAGCAATGGGGAAAGGATTCCCTATTTAA
TAAATGGTGCTGGGAAAACTGGCTAGCCATAGGTAGAAAGCTGAAACTGGACCCCTTCCTTACACCTTAT
ACAAAAATTAATTCAAGATGGATTAAAGACTTAAATGTTAGACCTAAAACCATAAAAACCCTAGAAGGAA
ACCTAGGTATTACCATTGAGGACACAGGCATGGGCAAGGACTTCATGTCTAAAACACCAAAAGCAATGGC
AACAAAAGACAAAATTGACAAATGGGATCTAATTAAACTAAAGAGCTTCTGCACAGCAAAAGAAACTACC
ATCAGAGTGAACAGGCAACCTACAAAATGGGAGAAACTTTTTGCAACCTATTCATCTGACAAAGGGCTAA
TATCCAGAATCTACAAAGAACTCAAACAAATTTACAAGAAAAGAACAAACAACCCCATCAAAAAAAAAAC
TAACAACCCCATCAAAAAGCGGGCAAAGGATATGAACAGACACTTCTCAAAAGAAGACATTTATGCAGCC
AAAAGACACATGAAAAAATGCTCATCATCACTGGCCATCAGAGAAATGCAAATGAAAACCACAATGAGAT
ACCATCTCACACCAGTTAGAATGGCGATCATTAAAAAGTCAGGAAACAACAGGTGCTGGAGAGGATGTGG
AGAAATAGGAACACTTTTACGCTGTTGGTGGGACTGTAAACTAGTTCAACCATTGTGGAAGACAGTGTGG
CGATTCCTCAGGGATCTAGAACTAGAAATACCATTTGACCCAGCCATCCCATTACTGGGTATATACCCAA
AGGATTATAAATCATGCTGCTATAAAGACACATGCAGACGTATGTTTATTGCGGCACTATTCACAATAGC
AAAGACTTGGAACCAACCCAAATGTCCAACAATGATAGACTGGATTAAGAAAATGTGGCACATATACACC
ATGAAATACTATGCAGCCATAAAAAATGATGAGTTCATGTCCTTTGTAGGGACATGGATGAAGCTGGAAA
CCATCATTCTCAGCAAACTATCACAAGGACAGAAAACCAAACACCACATGTTCTCACTCATAGGTGGAAA
TTGAACAATGAGAATACTTTGACACAGGAAGGGGAACATC

In a search of public sequence databases, the NOV66 nucleic acid sequence, located on chromsome 13 has 2399 of 2567 bases (93%) identical to a gb:GENBANK-ID:HSNOD1G2|acc:AF149774.1 mRNA from Homo sapiens (Homo sapiens NOD1 protein (NOD1) gene, exons 4 through 14 and complete cds). Public nucleotide databases include all GenBank databases and the GeneSeq patent database.

The disclosed NOV66 polypeptide (SEQ ID NO:156) encoded by SEQ ID NO:155 has 1018 amino acid residues and is presented in Table 66B using the one-letter amino acid code. Signal P, Psort and/or Hydropathy results predict that NOV66 has no signal peptide and is likely to be localized in the cytoplasm with a certainty of 0.6000

TABLE 66B
Encoded NOV66 protein sequence.

| | |
|---|---|
| MKTEIKMFFETNENKDTTYQNLWDTFKAVCRGKFTALNAHKRKQERSKIDTLTSQLKQLQ | (SEQ ID NO:156) |
| KQEQTLSKASRRQEITKIRAELKEIETQKTLQKINESRSWFFEKINKIDTLLARLIKKKR | |
| EKNQIDAIKNDKADITTDPTEIQTTIREYYKHLYANKLENLEEMDKFLDTYTLPRINQEE | |
| VESLNRPITGSEIEAIINSLPTKKSPGPDGFTAEFYQKYKEELIPFLLKLFQSIEKEGIL | |
| PNSFDEASIILIPKPGRDTTKKENFRPISLMNIDAKILNKILANRIKQHIKKLIHHDQVG | |
| FIPGMQGWFNICKSINVIQHINRTKDKNHMIISIDAEKAFDKIQQHFMLKTLNKLGIDGT | |
| YLKIIRAICDKPTANIILNGQKLEAFPLKTGTRQGCPLSPLLFNIVLEVLARAIRQEKEI | |
| KGIQLGKEEVKLSLFADDMIVYLENPIISAQNLLKLISNFSKVSGYKIDVQKSQAFLYTN | |
| TDQTESQIMSDLPFTIASKRIKYLGIQLTRDVKDLFKENYKPLLNEIKKDTNKWKNIPGS | |
| WIGRINIVKMAIEPKVIYRFNAIPIKLPMTFFTELEKTTLKFIWNQKRAHIAKSILNQKN | |
| KAGGITPPDFKLYYKATVNKTAWYWYQNRDIDQWNRTDPSEIMPHIYNYLIFDKPDKKKQ | |
| WGKDSLFNKWCWENWLAIGRKLKLDPFLTPYTKINSRWIKDLNVRPKTIKTLEGNLGITI | |
| EDTGMGKDFMSKTPKAMATKDKIDKWDLIKLKSFCTAKETTIRVNRQPTKWEKLFATYSS | |
| DKGLISRIYKELKQIYKKRTNNPIKKKTNNPIKKRAKDMNRHFSKEDIYAAKRHMKKCSS | |
| SLAIREMQMKTTMRYHLTPVRMAIIKKSGNNRCWRGCGEIGTLLRCWWDCKLVQPLWKTV | |
| WRFLRDLELEIPFDPAIPLLGIYPKDYKSCCYKDTCRRMFIAALFTIAKTWNQPKCPTMI | |
| DWIKKMWHIYTMKYYAAIKNDEFMSFVGTWMKLETIILSKLSQGQKTKHHMFSLIGGN | |

A search of sequence databases reveals that the NOV66 amino acid sequence has have 947 of 1018 amino acid residues (93%) identical to, and 965 of 1018 amino acid residues (94%) similar to, the 1010 amino acid residue patp:B38012 protein from human (Human secreted protein (L1H 3′ region)). Public amino acid databases include the GenBank databases, SwissProt, PDB and PIR.

The disclosed NOV66 polypeptide has homology to the amino acid sequences shown in the BLASTP data listed in Table 66C.

TABLE 66C
BLAST results for NOV66

| Gene Index/ | | Length | Identity | Positives | |
|---|---|---|---|---|---|
| Identifier | Protein/Organism | (aa) | (%) | (%) | Expect |
| gi\|106322\|pir\|\|B34087 | protein (L1H 3′ | 1280 | 955/1018 | 979/1018 | 0.0 |
| | region) - human | | (93%) | (95%) | |
| gi\|2072958\|gb\|AAC51267.1\| | putative p150 | 1275 | 945/1018 | 971/1018 | 0.0 |
| (U93567) | [Homo sapiens] | | (92%) | (94%) | |
| gi\|5052951\|gb\|AAD38785.1\| | unknown [Homo | 1275 | 948/1018 | 972/1018 | 0.0 |
| AF149422_2 | sapiens] | | (93%) | (95%) | |
| (AF149422) | | | | | |
| gi\|5070622\|gb\|AAD39215.1\| | unknown [Homo | 1275 | 945/1018 | 973/1018 | 0.0 |
| AF148856_2 | sapiens] | | (92%) | (94%) | |
| (AF148856) | | | | | |
| gi\|2072953\|gb\|AAC51264.1\| | p150 [Homo | 1275 | 943/1018 | 971/1018 | 0.0 |
| (U93565) | sapiens] | | (92%) | (94%) | |

Table 66D lists the domain descriptions from DOMAIN analysis results against NOV66. This indicates that the NOV66 sequence has properties similar to those of other proteins known to contain this domain.

TABLE 66D
Domain Analysis of NOV66

gnl|Pfam|pfam00078, rvt, Reverse transcriptase (RNA-dependent
DNA polymerase). A reverse transcriptase gene is usually
indicative of a mobile element such as a retrotransposon
or retrovirus. Reverse transcriptases occur in a variety of
mobile elements, including retrotransposons, retroviruses,
group II introns, bacterial msDNAs, hepadnaviruses, and
caulimoviruses.
CD-Length = 208 residues, 93.8% aligned
Score = 114 bits (285), Expect = 3e−26

The disclosed NOV66 nucleic acid of the invention encoding a secreted protein-like protein includes the nucleic acid whose sequence is provided in Table 66A or a fragment thereof. The invention also includes a mutant or variant nucleic acid any of whose bases may be changed from the corresponding base shown in Table 66A while still encoding a protein that maintains its secreted protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 7 percent of the bases may be so changed.

The disclosed NOV66 protein of the invention includes the secreted protein-like protein whose sequence is provided in Table 66B. The invention also includes a mutant or variant protein any of whose residues may be changed from the corresponding residue shown in Table 66B while still encoding a protein that maintains its secreted protein-like activities and physiological functions, or a functional fragment thereof. In the mutant or variant protein, up to about 7 percent of the residues may be so changed.

The invention further encompasses antibodies and antibody fragments, such as F_{ab} or (F_{ab})₂, that bind immunospecifically to any of the proteins of the invention.

The above defined information for this invention suggests that this secreted protein-like protein (NOV66) may function as a member of a “secreted protein family”. Therefore, the NOV66 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV66 nucleic acids and proteins of the invention are useful in potential therapeutic applications implicated in diseases including but not limited to various pathologies and disorders as indicated below. For example, a cDNA encoding the secreted protein-like protein (NOV66) may be useful in gene therapy, and the secreted protein-like protein (NOV66) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV66 nucleic acid encoding the secreted protein-like protein of the invention, or fragments thereof, may further be useful in diagnostic applications, wherein the presence or amount of the nucleic acid or the protein are to be assessed.

NOV66 nucleic acids and polypeptides are further useful in the generation of antibodies that bind immuno-specifically to the novel NOV66 substances for use in therapeutic or diagnostic methods. These antibodies may be generated according to methods known in the art, using prediction from hydrophobicity charts, as described in the “Anti-NOVX Antibodies” section below. The disclosed NOV66 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV67

NOV67 includes two acyltransferase-like proteins disclosed below. The disclosed sequences have been named NOV67a and NOV67b.

NOV67a

A disclosed NOV67a nucleic acid of 1116 nucleotides (also referred to as CG57823-01) encoding a acyltransferase-like protein is shown in Table 67A. The start and stop codons are in bold letters.

TABLE 67A
NOV67a nucleotide sequence.

| | |
|---|---|
| CCGGCACCGGCGTCAAGGCCATGGCGCTGTGCCCGGGATGGGTACACACCGAATTCCACT | (SEQ ID NO:157) |
| CACGCGCCAACGTCACCGGCAACCATCTGCCGGACTTTTTCTGGATCGACGCCGAAGTTC | |
| TGGTACGCGAGGCTCTCAACGACCTTGACCATGACAAGGTAGTATCCATTCCTACCCCGC | |
| TCTGGAAGTTCTTCATCGCAGTGGCCACACATACCCCACGTTCCGCTATGAGATTCCTGT | |
| CACGAACTCTGTCCTCGTCTCGAGACAAGGACGACCATCCTCGACACACTCCGGGAGGCG | |
| AGGCCTGAGATGGCCAGCGTCAAACCCACTAAGGACCGGGGCCGGTACACCAATGATCTG | |
| TCCGCCGCGACGCGGCAGGCAGCGAACATGCTTCTGCTGCGTCCTTTGGTGTGGAAAGTC | |
| GTCAAAGTGAGCGTCCACGGAGCCGACAACCTCGACGGGCTCGACGGTGCTTACGTCGCC | |
| GTCGCTAACCATTCCTCCCACCTCGACGCGCCGCTCGTTTTTGGGGCCCTTCCCAAGCGG | |
| CTGTCAAAGTACCTAGCTACCGGGGCCGCTGCTGACTATTTCTTCACCGCCTGGTGGAAG | |
| GCCATCGCTCCGGTGCTCTTCTTCAACGCGTTCCCGGTCGACCGAGGCAAAGGCAAAAGT | |
| AAGCAAGGTGCCCGTAGTCCCCGTTCCCACCGCGGTATGGCTGGGTCACTGCTGACAGAT | |
| GGCGTCCCCCTGCTGATCTTTCCGGAGGGCACCCGGTCTCGCACCGGCGCAATGGGCACC | |
| TTCAAACCTGGGGCTGCCGCATTGGCTATTTCACGTGGGGTTCCGGTTATCCCGATTGCT | |
| TTAGTAGGAGCATGGGCGGCTATGCCGTCCGAGCAAGCCAGGTTACCAAAAGGACGTCCA | |
| TTGGTCCACGTGGCTATTGGACACCCTATGGACCCTGTTCCCGGCGAGATCGCCCACCAA | |
| TTCTCCGAACGGATTCGTCGCCAGGTCATTGAGTTGCACGACCAAACCGCCCGCGCCTAC | |
| GGCATGCCAACCCTTGACGAATACGGACGCCACCGCGCGCTAAGCCAGGCCTCCGAGAGC | |
| GGCGACACCGCATCCACCAACCACTCGACGTGACAC | |

In a search of public sequence databases, the NOV67a nucleic acid sequence has 323 of 558 bases (57%) identical to a gb:GENBANK-ID:AF263912|acc:AF263912.1 mRNA from Streptomyces noursei (Streptomyces noursei ATCC 11455 nystatin biosynthetic gene cluster, complete sequence). Public nucleotide databases include all GenBank databases and the GeneSeq patent database.

The disclosed NOV67a polypeptide (SEQ ID NO:158) encoded by SEQ ID NO:157 has 267 amino acid residues and is presented in Table 67B using the one-letter amino acid code. Signal P, Psort and/or Hydropathy results predict that NOV67a has a signal peptide and is likely to be localized at the ER plasma membrane with a certainty of 0.85000. The most likely cleavage site for a NOV67a peptide is between amino acids 44 and 45.

TABLE 67B
Encoded NOV67a protein sequence.

| | |
|---|---|
| MASVKPTKDRGRYTNDLSAATRQAANMLLLRPLVWKVVKVSVHGADNLDGLDGAYVAVAN | (SEQ ID NO:158) |
| HSSHLDAPLVFGALPKRLSKYLATGAAADYFFTAWWKAIAPVLFFNAFPVDRGKGKSKQG | |
| ARSPRSHRGMAGSLLTDGVPLLIFPEGTRSRTGAMGTFKPGAAALAISRGVPVIPIALVG | |
| AWAAMPSEQARLPKGRPLVHVAIGHPMDPVPGEIAHQFSERIRRQVIELHDQTARAYGMP | |
| TLDEYGRHRALSQASESGDTASTNHST | |

A search of sequence databases reveals that the NOV67a amino acid sequence has 65 of 181 amino acid residues (35%) identical to, and 96 of 181 amino acid residues (53%) similar to, the 240 amino acid residue ptnr:TREMBLNEW-ACC:CAC01452 protein from Streptomyces coelicolor (PUTATIVE ACYLTRANSFERASE). Public amino acid databases include the GenBank databases, SwissProt, PDB and PIR.

NOV67a is expressed in at least Bone, Bone Marrow, Brain, Liver, Lung, Lymph node, Placenta, Prostate, Thalamus, Thyroid and Uterus. This information was derived by determining the tissue sources of the sequences that were included in the invention including but not limited to SeqCalling sources, Public EST sources, Literature sources, and/or RACE sources.

NOV67b

A disclosed NOV67b nucleic acid of 906 nucleotides (also referred to as CG57823-02) encoding a acyltransferase-like protein is shown in Table 67C. The start and stop codons are in bold letters.

TABLE 67C
NOV67b nucleotide sequence.

| | |
|---|---|
| ATACCCCACGTTCCGCTATGAGATTCCTGTCACGAACTCTGTCCTCGTCTCGAGACAAGG | (SEQ ID NO:159) |
| ACGACCACCCTCGACACACTCCGGGAGGCGAGGCCTGAGATGGCCAGCGTCAAACCCACT | |
| AAGGACCGGGGCCGGTACACCAATGATCTGTCCGCCGCGACGCGGCAGGCAGCGAACATG | |
| CTTCTGCTGCGTCCTTTGGTGTGGAAAGTCGTCAAAGTGAGCGTCCACGGAGCCGACAAC | |
| CTCGACGGGCTCGACGGTGCCTACGTCGCCGTCGCTAACCATTCCTCCCACCTCGACGCG | |
| CCGCTCGTTTTTGGGGCCCTTCCCAAGCGGCTGTCAAAGTACCTAGCTACCGGGGCCGCT | |
| GCTGACTATTTCTTCACCGCCTGGTGGAAGGCCATCGCTCCGGTGCTCTTCTTCAACGCG | |
| TTCCCGGTCGACCGAGGCAAAGGCAAAAGTAAGCAAGGTGCCCGTAGTCCCCGTTCCCAC | |
| CGCGGTATGGCTGGGTCACTGCTGACAGATGGCGTCCCCCTGCTGATCTTTCCGGAGGGC | |
| ACCCGGTCTCGCACCGGTGCAATGGGCACCTTCAAACCTGGGGCTGCCGCATTGGCTATT | |
| TCACGTGGGGTTCCGGTTATCCCGATTGCTTTAGTAGGAGCATGGGCGGCTATGCCGTCC | |
| GAGCAAGCCGGGTTACCAAAAGGACGCCCATCGGTCCACGTGGCTATTGGACACCCTATG | |
| GACCCTGTTCCCGGCGAGATCGCCCACCAATTCTCCGAACGGATTCGTCGCCAGGTCATT | |
| CACCGCGCGCTAAGCCAGGCCTCCGAGAGCGGCGACACCGCATCCACCAACCACTCGACG | |
| TGACAC | |

In a search of public sequence databases, the NOV nucleic acid sequence has 323 of 558 bases (57%) identical to a gb:GENBANK-ID:AF263912|acc:AF263912.1 mRNA from Streptomyces noursei (Streptomyces noursei ATCC 11455 nystatin biosynthetic gene cluster, complete sequence). Public nucleotide databases include all GenBank databases and the GeneSeq patent database.

The disclosed NOV67b polypeptide (SEQ ID NO:160) encoded by SEQ ID NO:159 has 267 amino acid residues and is presented in Table 67D using the one-letter amino acid code. Signal P, Psort and/or Hydropathy results predict that NOV67b has a signal peptide and is likely to be localized at the ER plasma membrane with a certainty of 0.85000. The most likely cleavage site for a NOV67b peptide is between amino acids 44 and 45.

TABLE 67D
Encoded NOV67b protein sequence.

| | |
|---|---|
| MASVKPTKDRGRYTNDLSAATRQAANMLLLRPLVWKVVKVSVHGADNLDGLDGAYVAVAN | (SEQ ID NO:160) |
| HSSHLDAPLVFGALPKRLSKYLATGAAADYFFTAWWKAIAPVLFFNAFPVDRGKGKSKQG | |
| ARSPRSHRGMAGSLLTDGVPLLIFPEGTRSRTGAMGTFKPGAAALAISRGVPVIPIALVG | |
| AWAAMPSEQAGLPKGRPSVHVIAGHPMDPVPGEIAHQFSERIRRQVIELHDQTARAYGMP | |
| TLDEYGRHRALSQASESGDTASTNHST | |

A search of sequence databases reveals that the NOV67b amino acid sequence has 65 of 181 amino acid residues (35%) identical to, and 96 of 181 amino acid residues (53%) similar to, the 240 amino acid residue ptnr:TREMBLNEW-ACC:CAC01452 protein from Streptomyces coelicolor (PUTATIVE ACYLTRANSFERASE). Public amino acid databases include the GenBank databases, SwissProt, PDB and PIR.

NOV67b is expressed in at least Bone, Bone Marrow, Brain, Liver, Lung, Lymph node, Placenta, Prostate, Thalamus, Thyroid, Uterus. This information was derived by determining the tissue sources of the sequences that were included in the invention including but not limited to SeqCalling sources, Public EST sources, Literature sources, and/or RACE sources.

The disclosed NOV67 polypeptide has homology to the amino acid sequences shown in the BLASTP data listed in Table 67E.

TABLE 67E
BLAST results for NOV67

| Gene Index/ | | Length | Identity | Positives | |
|---|---|---|---|---|---|
| Identifier | Protein/Organism | (aa) | (%) | (%) | Expect |
| gi\|15644441\|ref\|NP_229493.1\| | 1-acyl-sn- | 247 | 64/214 | 100/214 | 3e−14 |
| (NC_000853) | glycerol-3- | | (29%) | (45%) | |
| | phosphate | | | | |
| | acetyltransferase, | | | | |
| | putative | | | | |
| | [Thermotoga | | | | |
| | maritima] | | | | |
| gi\|9716114\|emb\|CAC01452.1\| | putative | 240 | 63/184 | 92/184 | e−12 |
| (AL391014) | acyltransferase | | (34%) | (49%) | |
| | [Streptomyces | | | | |
| | coelicolor A3 (2)] | | | | |
| gi\|11693120\|gb\|AAG38841.1\| | putative acetyl | 249 | 68/203 | 94/203 | 4e−12 |
| (AY010120) | transferase | | (33%) | (45%) | |
| | [Xanthomonas | | | | |
| | oryzae pv. | | | | |
| | oryzae] | | | | |
| gi\|15607028\|ref\|NP_ | 2- | 211 | 59/199 | 92/199 | e−11 |
| 214410.1\| | acylglycerophosph | | (29%) | (45%) | |
| (NC_000918) | oethanolamine | | | | |
| | acyltransferase | | | | |
| | [Aquifex | | | | |
| | aeolicus] | | | | |
| gi\|15606303\|ref\|NP_ | long-chain-fatty- | 823 | 47/149 | 72/149 | 3e−10 |
| 213682.1\| | acid CoA ligase | | (31%) | (47%) | |
| (NC_000918) | [Aquifex | | | | |
| | aeolicus] | | | | |

Tables 67F-G list the domain descriptions from DOMAIN analysis results against NOV67. This indicates that the NOV67 sequence has properties similar to those of other proteins known to contain this domain.

TABLE 67F
Domain Analysis of NOV67

gnl|Pfam|pfam01553, Acyltransferase, Acyltransferase.
This family contains acyltransferases involved in phospholipid
biosynthesis and other proteins. This family also includes
tafazzin, the Barth syndrome gene.
CD-Length = 185 residues, 95.7% aligned
Score = 92.4 bits (228), Expect = 3e−20

TABLE 67G
Domain Analysis of NOV67

gnl|Smart|smart00563, PlsC, Phosphate acyltransferases;
Function in phospholipid biosynthesis and have either
glycerolphosphate, 1-acylglycerolphosphate, or 2-
acylglycerolphosphoethanolamine acyltransferase activities.
Tafazzin, the product of the gene mutated in patients with Barth
syndrome, is a member of this family.
CD-Length = 118 residues, 99.2% aligned
Score = 81.6 bits (200), Expect = 5e−17

The polyene macrolide antibiotic nystatin produced by Streptomyces noursei ATCC 11455 is an important antifungal agent. The nystatin molecule contains a polyketide moiety represented by a 38-membered macrolactone ring to which the deoxysugar mycosamine is attached. Molecular cloning and characterization of the genes governing the nystatin biosynthesis is of considerable interest because this information can be used for the generation of new antifungal antibiotics.

Glycerol-3-phosphate 1-acyltransferase (E.C. 2.3.1.15; G3PAT) catalyses the incorporation of an acyl group from either acyl-acyl carrier proteins (acylACPs) or acylCoAs into the sn-1 position of glycerol 3-phosphate to yield 1-acylglycerol 3-phosphate. Crystals of squash G3PAT have been obtained by the hanging-drop method of vapour diffusion using PEG 4000 as the precipitant. These crystals are most likely to belong to space group P2(1)₂(1)₂(I), with approximate unit-cell parameters a=61.1, b=65.1, c=103.3 A, alpha=beta=gamma=90 degrees and a monomer in the asymmetric unit. X-ray diffraction data to 1.9 A resolution have been collected in-house using a MAR 345 imaging-plate system. PMID: 11223529.

Exposure of heparinized human plasma to gas-phase cigarette smoke produced a dose-dependent reduction in the activity of platelet-activating factor acetylhydrolase (PAF-AH). Reductions of nearly 50% in PAF-AH activity were observed following exposure to gas-phase smoke from four cigarettes over an 8-h period. During this time of exposure, lecithin:cholesterol acyltransferase (LCAT) was rendered almost completely inactive (>80%). In contrast, paraoxonase was totally unaffected by cigarette smoke. Supplementation of plasma with 1 mM reduced glutathione was found to protect both PAF-AH and LCAT from cigarette smoke, suggesting that cysteine modifications may have contributed to the inhibition of these two enzymes.

Although the atheroprotective role of high-density lipoprotein (HDL) has been well documented in epidemiological and animal studies, highly effective therapeutic approaches for the selective increase of plasma HDL levels or function are not yet available. Several mechanisms by which HDL exerts an atheroprotective effect have been proposed on the basis of experiments in vitro and in vivo. These mechanisms include directing excess cellular cholesterol from the peripheral tissues to the liver in ‘reverse cholesterol transport’, inhibiting oxidative modification or aggregation of LDL, and modulating inflammatory responses to favour vasoprotection. High density lipoproteins (HDL) mediate reverse cholesterol transport as well as the clearance of oxidation

The disclosed NOV67 nucleic acid of the invention encoding a acyltransferase-like protein includes the nucleic acid whose sequence is provided in Table 67A or a fragment thereof. The invention also includes a mutant or variant nucleic acid any of whose bases may be changed from the corresponding base shown in Table 67A while still encoding a protein that maintains its acyltransferase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 43 percent of the bases may be so changed.

The disclosed NOV67 protein of the invention includes the acyltransferase-like protein whose sequence is provided in Table 67B. The invention also includes a mutant or variant protein any of whose residues may be changed from the corresponding residue shown in Table B while still encoding a protein that maintains its acyltransferase-like activities and physiological functions, or a functional fragment thereof. In the mutant or variant protein, up to about 65 percent of the residues may be so changed.

The invention further encompasses antibodies and antibody fragments, such as F_{ab} or (F_{ab})₂, that bind immunospecifically to any of the proteins of the invention.

The above defined information for this invention suggests that this acyltransferase-like protein (NOV67) may function as a member of a “acyltransferase family”. Therefore, the NOV67 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV67 nucleic acids and proteins of the invention are useful in potential therapeutic applications implicated in diseases including but not limited to various pathologies and disorders as indicated below. For example, a cDNA encoding the acyltransferase-like protein (NOV67) may be useful in gene therapy, and the acyltransferase-like protein (NOV67) may be useful when administered to a subject in need thereof. By way of nonlimiting example, the compositions of the present invention will have efficacy for treatment of patients suffering from Osteoporosis, Hypercalceimia, Arthritis, Ankylosing spondylitis, Scoliosis, Hemophilia, hypercoagulation, Idiopathic thrombocytopenic purpura, autoimmune disease, allergies, immunodeficiencies, transplantation, Graft vesus host, Von Hippel-Lindau (VHL) syndrome, Cirrhosis, Transplantation, Lymphedema, Allergies, Hemophilia, hypercoagulation, Idiopathic thrombocytopenic purpura, immunodeficiencies, Fertility, Osteoporosis, Hypercalceimia, Arthritis, Ankylosing spondylitis, Scoliosis, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, or other pathologies or conditions. The NOV67 nucleic acid encoding the acyltransferase-like protein of the invention, or fragments thereof, may further be useful in diagnostic applications, wherein the presence or amount of the nucleic acid or the protein are to be assessed.

NOV67 nucleic acids and polypeptides are further useful in the generation of antibodies that bind immuno-specifically to the novel NOV67 substances for use in therapeutic or diagnostic methods. These antibodies may be generated according to methods known in the art, using prediction from hydrophobicity charts, as described in the “Anti-NOVX Antibodies” section below. The disclosed NOV67 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV68

A disclosed NOV68 nucleic acid of 1388 nucleotides (also referred to as CG57801-01) encoding a guanine nucleotide exchange factor-like protein is shown in Table 68A. The start and stop codons are in bold letters.

TABLE 68A
NOV68 nucleotide sequence.

| | |
|---|---|
| AACAGGTCACAGGAAAGTAGGAACCTGGGAGGTCTCCTGCCTCTGCTCCATGCTGCCCTC | (SEQ ID NO:161) |
| CCCTGCCCCAAGTCACCTGTCCCCTGTATGTGGGTTGCAGTTGCGAGTGAATCAGGAAGA | |
| GCTGTCGGAAAACTCCAGCAGCACCCCCAGTGAGGAGCAGGACGAGGAGGCCAGCCAGAG | |
| CCGCCACAGACACTGTGAGAACAAGCAGCAGATGCGGACCAACGTCATCCGGGAGATCAT | |
| GGACACCGAGCGGGTGTACATCAAACACCTCAGGGACATCTGTGAGGGCTATATCCGACA | |
| GTGCCGCAAGCACACAGGAATGTTCACCGTTGCGCAGCTAGCCACTATTTTTGGAAACAT | |
| TGAAGATATTTACAAATTCCAAAGAAAGTTTCTGAAAGACCTTGAGAAACAGTACAACAA | |
| AGAGGAACCTCACTTAAGTGAAATAGGATCTTGCTTTCTTCAAAATCAAGAGGGCTTTGC | |
| CATCTATTCCGAGTACTGCAACAACCACCCGGGCGCCTGCCTGGAGCTCGCCAACCTCAT | |
| GAAGCAGGGCAAGTACAGACATTTCTTTGAAGCCTGCCGCCTGCTGCAGCAGATGATTGA | |
| CATCGCCATCGACGGGTTCCTGCTCACACCAGTGCAGAAGATCTGCAAATACCCGCTGCA | |
| GCTGGCCGAGCTGCTCAAGTATACCACACAGGAACACAGTGATTACAGCAACATAAAGGC | |
| AGCATATGAGGCCATGAAGAATGTGGCCTGTCTGATCAACGAGCGCAAGCGCAAGCTGGA | |
| GAGCATCGACAAGATAGCTCGCTGGCAGGTGTCTATCGTGGGCTGGGAGGGACTGGATAT | |
| CTTAGACCGAAGCTCAGAATTGATTCATTCTGGGGAGCTGACCAAAATCACTAAGCAAGG | |
| CAAAAGCCAGCAGCGGACGTTCTTCCTGTTTGACCACCAGCTGGTGTCCTGCAAGAAGGA | |
| CCTGCTGCGCAGGGACATGCTGTACTACAAGGGCCGGCTGGACATGGATGAGATGGAGCT | |
| TGTGGACCTGGGGGATGGGCGCGACAAGGACTGCAACCTCAGCGTGAAAAATGCCTTCAA | |
| GCTCGTCAGTAGGACCACAGACGAGGTTTATTTGTTTTGTGCCAAAAAACAAGAAGACAA | |
| GGCGAGGTGGCTGCAGGCCTGTGCAGATGAAAGGAGGCGGGTGCAAGAGGACAAGGAGAT | |
| GGGAATGGAAATTTCAGAAAACCAGAAGAAACTTGCCATGTTAAATGCTCAAAAGGCAGG | |
| ACATGGAAAGTCAAAAGGTAAGTTATGGAGAAGGCTTTGTCCCCTTAATGCTTATCAGTA | |
| TTCTCCTGAAAATGGGAGCATACCCCAAGTTGTCAGCCTGTGACCAGCTTGGAGCAAGGA | |
| GAACAGTA | |

In a search of public sequence databases, the NOV68 nucleic acid sequence, located on chromsome 13 has has 822 of 1143 bases (71%) identical to a gb:GENBANK-ID:AB029035|acc:AB029035.1 mRNA from Homo sapiens (Homo sapiens mRNA for KIAA1112 protein, partial cds). Public nucleotide databases include all GenBank databases and the GeneSeq patent database.

The disclosed NOV68 polypeptide (SEQ ID NO:162) encoded by SEQ ID NO:161 has 437 amino acid residues and is presented in Table 68B using the one-letter amino acid code. Signal P, Psort and/or Hydropathy results predict that NOV68 has localized in the cytoplasm with a certainty of 0.3000.

TABLE 68B
Encoded NOV68 protein sequence.

| | |
|---|---|
| MLPSPAPSHLSPVCGLQLRVNQEELSENSSSTPSEEQDEEASQSRHRHCENKQQMRTNVI | (SEQ ID NO:162) |
| REIMDTERVYIKHLRDICEGYIRQCRKHTGMFTVAQLATIFGNIEDIYKFQRKFLKDLEK | |
| QYNKEEPHLSEIGSCFLQNQEGFAIYSEYCNNHPGACLELANLMKQGKYRHFFEACRLLQ | |
| QMIDIAIDGFLLTPVQKICKYPLQLAELLKYTTQEHSDYSNIKAAYEAMKNVACLINERK | |
| RKLESIDKIARWQVSIVGWEGLDILDRSSELIHSGELTKITKQGKSQQRTFFLFDHQLVS | |
| CKKDLLRRDMLYYKGRLDMDEMELVDLGDGRDKDCNLSVKNAFKLVSRTTDEVYLFCAKK | |
| QEDKARWLQACADERRRVQEDKEMGMEISENQKKLAMLNAQKAGHGKSKGKLWRRLCPLN | |
| AYQYSPENGSIPQVVSL | |

A search of sequence databases reveals that the NOV68 amino acid sequence has 253 of 402 amino acid residues (62%) identical to, and 317 of 402 amino acid residues (78%) similar to, the 493 amino acid residue ptnr:SPTREMBL-ACC:Q9QX73 protein from Rattus norvegicus (Rat) (COLLYBISTIN I). Public amino acid databases include the GenBank databases, SwissProt, PDB and PIR.

NOV68 is expressed in at least Kidney, Pituitary Gland, Placenta, Uterus, Aorta, Hypothalamus, Pancreas, Spleen, Epidermis, Muscle, Spinal Cord. This information was derived by determining the tissue sources of the sequences that were included in the invention including but not limited to SeqCalling sources, Public EST sources, Literature sources, and/or RACE sources.

The disclosed NOV68 polypeptide has homology to the amino acid sequences shown in the BLASTP data listed in Table 68C.

TABLE 68C
BLAST results for NOV68

| Gene Index/ | | Length | Identity | Positives | |
|---|---|---|---|---|---|
| Identifier | Protein/Organism | (aa) | (%) | (%) | Expect |
| gi\|18581232\|ref\|XP_ | similar to Rho | 652 | 393/399 | 395/399 | 0.0 |
| 062774.2\| | guanine | | (98%) | (98%) | |
| (XM_062774) | nucleotide | | | | |
| | exchange factor | | | | |
| | 4, isoform a; | | | | |
| | APC-stimulated | | | | |
| | guanine | | | | |
| | nucleotide | | | | |
| | exchange factor | | | | |
| | [Homo sapiens] | | | | |
| gi\|8809845\|gb\|AAF79955.1\| | RhoGEF [Homo | 720 | 256/429 | 319/429 | e−141 |
| AF249745_1 | sapiens] | | (59%) | (73%) | |
| (AF249745) | | | | | |
| gi\|5689561\|dbj\|BAA83064.1\| | KIAA1112 protein | 694 | 250/402 | 311/402 | e−140 |
| (AB029035) | [Homo sapiens] | | (62%) | (77%) | |
| gi\|13027402\|ref\|NP_ | collybistin I | 493 | 254/407 | 318/407 | e−140 |
| 076447.1\| | [Rattus | | (62%) | (77%) | |
| (NM_023957) | norvegicus] | | | | |
| gi\|7662108\|ref\|NP_056000.1\| | Cdc42 guanine | 516 | 250/397 | 312/397 | e−140 |
| (NM_015185) | exchange factor | | (62%) | (77%) | |
| | 9; Cdc42 guanine | | | | |
| | exchange factor | | | | |
| | (GEF) 9 [Homo | | | | |
| | sapiens] | | | | |

Table 68D-E lists the domain descriptions from DOMAIN analysis results against NOV68. This indicates that the NOV68 sequence has properties similar to those of other proteins known to contain this domain.

TABLE 68D
Domain Analysis of NOV68

gnl|Smart|smart00325, RhoGEF, Guanine nucleotide exchange
factor for Rho/Rac/Cdc42-like GTPases; Guanine nucleotide
exchange factor for Rho/Rac/Cdc42-like GTPases Also called Dbl-
homologous (DH) domain. It appears that PH domains invariably
occur C-terminal to RhoGEF/DH domains. Improved coverage.
CD-Length = 181 residues, 100.0% aligned
Score = 156 bits (394), Expect = 3e−39

TABLE 68E
Domain Analysis of NOV68

gnl|Smart|smart00233, PH, Pleckstrin homology domain.;
Domain commonly found in eukaryotic signalling proteins. The
domain family possesses multiple functions including the
abilities to bind inositol phosphates, and various proteins.
PH domains have been found to possess inserted domains (such
as in PLC gamma, syntrophins) and to be inserted within other
domains. Mutations in Brutons tyrosine kinase (Btk) within
its PH domain cause X-linked agammaglobulinaemia (XLA) in
patients. Point mutations cluster into the positively charged
end of the molecule around the predicted binding site for
phosphatidylinositol lipids.
CD-Length = 104 residues, 92.3% aligned
Score = 56.2 bits (134), Expect = 4e−09

The novel protein described in this application belongs to the guanine nucleotide exchange factor family of proteins which play a significant role in signal transduction. The guanine nucleotide exchange factor (GEF) domain that regulates GTP binding protein signaling. The GEF domain regulates positively the signaling cascades that utilize GTP-binding proteins (such as those of the ras superfamily) that function as molecular switches in fundamental events such as signal transduction, cytoskeleton dynamics and intracellular trafficking. Experiments have shown that the GEF and (PH) domains of FGD1 (faciogenital dyplasia protein (FGD1)) can bind specifically to the Rho family GTPase Cdc42Hs and stimulates the GDP-GTP exchange of the isoprenylated form of Cdc42Hs. The GEF domain of FGD1 has also been shown to activate 2 kinases involved in cell proliferation; the Jun NH2-terminal kinase and the p70 S6 kinase (See Zheng et. al.; J. Biol. Chem Dec. 27, 1996;271(52):33169-72). Thus this novel protein may play an important role in normal development as well as disease. This class of molecules (GEFs) is also being considered as a good drug target as the guanine nucleotide exchange factor RasGRP is a high-affinity target for diacylglycerol and phorbol esters and is bound by bryostatin 1, a compound currently in clinical trials (See Lorenzo et. al.; Mol. Pharmacol 2000 May;57(5):840-6). Collybistin I and II, which belong to the family of dbl-like GDP/GTP exchange factors (GEFs) are most homologous to the protein described in this application. Collybistin II regulates the membrane deposition of gephyrin (an integral membrane protein) by activating a GTPase of the Rho/Rac family and may be an important determinant of inhibitory postsynaptic membrane formation and plasticity (See Kins et. al. Nat. Neurosci 2000 January;3(1):22-9).

The disclosed NOV68 nucleic acid of the invention encoding a guanine nucleotide exchange factor-like protein includes the nucleic acid whose sequence is provided in Table 68A or a fragment thereof. The invention also includes a mutant or variant nucleic acid any of whose bases may be changed from the corresponding base shown in Table 68A while still encoding a protein that maintains its guanine nucleotide exchange factor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, tip to about 29 percent of the bases may be so changed.

The disclosed NOV68 protein of the invention includes the guanine nucleotide exchange factor-like protein whose sequence is provided in Table 68B. The invention also includes a mutant or variant protein any of whose residues may be changed from the corresponding residue shown in Table 68B while still encoding a protein that maintains its guanine nucleotide exchange factor-like activities and physiological functions, or a functional fragment thereof. In the mutant or variant protein, up to about 38 percent of the residues may be so changed.

The invention further encompasses antibodies and antibody fragments, such as F_{ab} or (F_{ab})₂, that bind immunospecifically to any of the proteins of the invention.

The above defined information for this invention suggests that this guanine nucleotide exchange factor-like protein (NOV68) may function as a member of a “guanine nucleotide exchange factor family”. Therefore, the NOV68 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV68 nucleic acids and proteins of the invention are useful in potential therapeutic applications implicated in diseases including but not limited to various pathologies and disorders as indicated below. For example, a cDNA encoding the guanine nucleotide exchange factor-like protein (NOV68) may be useful in gene therapy, and the guanine nucleotide exchange factor-like protein (NOV68) may be useful when administered to a subject in need thereof. By way of nonlimiting example, the compositions of the present invention will have efficacy for treatment of patients suffering from cancer, trauma, regeneration (in vitro and in vivo), viral/bacterial/parasitic infections, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, muscular dystrophy, myasthenia gravis, atherosclerosis, aneurysm, hypertension, fibromuscular dysplasia, stroke, scleroderma, obesity, transplantation, or other pathologies or conditions. The NOV68 nucleic acid encoding the guanine nucleotide exchange factor-like protein of the invention, or fragments thereof, may further be useful in diagnostic applications, wherein the presence or amount of the nucleic acid or the protein are to be assessed.

NOV68 nucleic acids and polypeptides are further useful in the generation of antibodies that bind immuno-specifically to the novel NOV68 substances for use in therapeutic or diagnostic methods. These antibodies may be generated according to methods known in the art, using prediction from hydrophobicity charts, as described in the “Anti-NOVX Antibodies” section below. The disclosed NOV68 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV69

NOV69 includes two aspartate aminotransferase-like proteins disclosed below. The disclosed sequences have been named NOV69a and NOV69b.

NOV69a

A disclosed NOV60a nucleic acid of 1463 nucleotides (also referred to as CG57719-01) encoding a aspartate aminotransferase-like protein is shown in Table 69A. The start and stop codons are in bold letters.

TABLE 69A
NOV69a nucleotide sequence.

| | |
|---|---|
| GGAAGACTTCTGGGCAGAAGCGGAACACAGGAGCAGAGACACATAGTCTTGGCTCCAGTT | (SEQ ID NO:163) |
| TCGTTTCAGTTATGCCCACCCTTTCAGTGTTCATGGATGTGCCCCTCGCCCACAAGCTAG | |
| AGGGCAGCTTGTTAAAGACCTACAAACAAGATGATTACCCGAACAAGATATTCTTAGCCT | |
| ATAGAGGCACCTTCCCACAGCCCCATGGAGTCCAGGAGAGATTTGTTTGCAGGCTGTCTG | |
| CAGAGCTCAGCCCTGGGGGCCCAAACCAGGCATCTGGAGCTCCCTCTGTGGTTTTCCTCA | |
| CAGTCTGCATGACAAATGAAGGCCATCCCTGGGTTTCTCTCGTGGTGCAGAAGACTCGAC | |
| TACAGATTTCACAGGATCCCTCCCTGAATTATGAGTACTTGCCCACCATGGGCCTGAAAT | |
| CATTCATCCAGGCCTCTCTAGCACTCCTCTTTGGAAAGCACAGCCAAGCCATTGTGGAGA | |
| ACAGGGTAGGGGGTGTACACACTGTTGGTGACAGTGGTGCCTTCCAGCTTGGCGTCCAGT | |
| TTCTCAGAGCTTGGCATAAGGATGCTCGTATAGTTTACATCATCTCTTCTCAAAAAGTTC | |
| CCACAGAACTGCATGGACTCGTCTTCCAGGACATGGGCTTTACAGTTTATGAATACTCTG | |
| TCTGGGACCCCAAGAAGCTATGCATGGACCCCGACATACTCCTCAATGTGGTGGAGCAGA | |
| TCCCACATGGCTGTGTCCTTGTGATGGGGAACATTATCGACTGCAAGTTGACACCAAGTG | |
| GGTGGGCAAAGTTGATGTCCATGATAAAGAGCAAGCAGATATTCCCATTTTTTGATATTC | |
| CCTGTCAAGGTTTATACACCAGTGACTTGGAAGAAGATACTAGAATCTTACAATACTTTG | |
| TGTCTCAAGGCTTTGAGTTCTTCTGCAGCCAGTCTCTGTCCAAAAATTTTGGCATTTATG | |
| ATGAAGGAGTGGGGATGCTAGTGGTGGTGGCAGTCAACAACCAGCAGCTGCTGTGTGTCC | |
| TCTCCCAGCTGGAAGGATTAGCCCAGGCCCTGTGGCTAAACCCCCCCAACACGGGTGCAC | |
| GTGTCATCACCTCCATCCTCTGCAACCCTGCTCTGCTGGGAGAATGGAAGCAGAGTCTAA | |
| AAGAAGTTGTAGAGAACATCATGCTAACCAAGGAAAAAGTGAAGGAGAAACTCCAGCTCC | |
| TGGGAACCCCTGGGTCCTGGGGTCACATCACCGAGCAGAGTGGGACCCACGGCTATCTTG | |
| GACTCAACTGTAAGCAGGTGGAATACCTGGTCAGGAAGAAGCACATCTATATCCCCAAGA | |
| ACGGTCAGATTAACTTCAGCTGTATCAATGCCAACAACATAAATTACATCACTGAGGGCA | |
| TCAATGAGGCTGTCCTCCTCACAGAGAGCTCAGAGATGTGTCTTCCAAAGGAAAAAAAAA | |
| CACTGATTGGAATAAAACTTTAG | |

In a search of public sequence databases, the NOV69a nucleic acid sequence, located on chromsome 8 has 316 of 327 bases (96%) identical to a gb:GENBANK-ID:AP00050|acc:AP000501.1 mRNA from Homo sapiens (Homo sapiens genomic DNA, chromosome 8p11.2, clone:91h23 to 9-41). Public nucleotide databases include all GenBank databases and the GeneSeq patent database.

The disclosed NOV69a polypeptide (SEQ ID NO:164) encoded by SEQ ID NO:163 has 463 amino acid residues and is presented in Table 69B using the one-letter amino acid code. Signal P, Psort and/or Hydropathy results predict that NOV69a has no signal peptide and is likely to be localized in the cytoplasm with a certainty of 0.3696.

TABLE 69B
Encoded NOV69a protein sequence.

| | |
|---|---|
| MPTLSVFMDVPLAHKLEGSLLKTYKQDDYPNKIFLAYRGTFPQPHGVQERFVCRLSAELS | (SEQ ID NO:164) |
| PGGPNQASGAPSVVFLTVCMTNEGHPWVSLVVQKTRLQISQDPSLNYEYLPTMGLKSFIQ | |
| ASLALLFGKHSQAIVENRVGGVHTVGDSGAFQLGVQFLRAWHKDARIVYIISSQKVPTEL | |
| HGLVFQDMGFTVYEYSVWDPKKLCMDPDILLNVVEQIPHGCVLVMGNIIDCKLTPSGWAK | |
| LMSMIKSKQIFPFFDIPCQGLYTSDLEEDTRILQYFVSQGFEFFCSQSLSKNFGIYDEGV | |
| GMLVVVAVNNQQLLCVLSQLEGLAQALWLNPPNTGARVITSILCNPALLGEWKQSLKEVV | |
| ENIMLTKEKVKEKLQLLGTPGSWGHITEQSGTHGYLGLNCKQVEYLVRKKHIYIPKNGQI | |
| NFSCINANNINYITEGINEAVLLTESSEMCLPKEKKTLIGIKL | |

A search of sequence databases reveals that the NOV69a amino acid sequence has 163 of 228 amino acid residues (71%) identical to, and 187 of 228 amino acid residues (82%) similar to, the 264 amino acid residue ptnr:TREMBLNEW-ACC:BAB24820 protein from Mus musculus (Mouse) (ADULT MALE TESTIS CDNA, RIKEN FULL-LENGTH ENRICHED LIBRARY, CLONE:1700083M11, FULL INSERT SEQUENCE). Public amino acid databases include the GenBank databases, SwissProt, PDB and PIR.

NOV69a is expressed in at least testis. This information was derived by determining the tissue sources of the sequences that were included in the invention including but not limited to SeqCalling sources, Public EST sources, Literature sources, and/or RACE sources.

NOV69b

A disclosed NOV69b nucleic acid of 1280 nucleotides (also referred to as CG57719-02) encoding a aspartate aminotransferase-like protein is shown in Table 69C. The start and stop codons are in bold letters.

TABLE 69C
NOV69b nucleotide sequence.

| | |
|---|---|
| CCACCCTTTCAGTTATGCCCACCCTTTCAGTGTTCATGGATGTGCCCCTCGCCCACAAGC | (SEQ ID NO:165) |
| TAGAGGGCAGCTTGTTAAAGACCTACAAACAAGATGATTACCCGAACAAGATATTCTTAG | |
| CCTATAGAGTCTGCATGACAAATGAAGGCCATCCCTGGGTTTCTCTCGTGGTGCAGAAGA | |
| CTCGACTACAGATTTCACAGGATCCCTCCCTGAATTATGAGTACTTGCCCACCATGGGCC | |
| TGAAATCATTCATCCAGGCCTCTCTAGCACTCCTCTTTGGAAAGCACAGCCAAGCCATTG | |
| TGGAGAACAGGGCAGGGGGTGTACACACTGTTGGTGACAGTGGTGCCTTCCAGCTTGGCG | |
| TCCAGTTTCTCAGAGCTTGGCATAAGGATGCTCGTATAGTTTACATCATCTCTTCTCAAA | |
| AAGAACTGCATGGACTCGTCTTCCAGGACATGGGCTTTACAGTTTATGAATACTCTGTCT | |
| GGGACCCCAAGAAGCTATGCATGGACCCCGACATACTCCTCAATGTGGTGGAGCAGATCC | |
| CACATGGCTGTGTCCTTGTGATGGGGAACATTATCGACTGCAAGTTGACACCAAGTGGGT | |
| GGGCAAAGTTGATGTCCATGATAAAGAGCAAGCAGATATTCCCATTTTTTGATATTCCCT | |
| GTCAAGGTTTATACACCAGTGACTTGGAAGAAGATACTAGAATCTTACAATACTTTGTGT | |
| CTCAAGGCTTTGAGTTCTTCTGCAGCCAGTCTCTGTCCAAGAATTTTGGCATTTATGATG | |
| AAGGAGTGGGGATGCTAGTGGTGGTGGCAGTCAACAACCAGCAGCTGCTGTGTGTCCTCT | |
| CCCAGCTGGAAGGATTAGCCCAGGCCCTATGGCTAAACCCCCCCAACACGGGTGCACGTG | |
| TCATCACCTCCATCCTCTGCAACCCTGCTCTGCTGGGAGAATGGAAGCAGAGTCTAAAAG | |
| AAGTTGTAGAGAACATCATGCTAACCAAGGAAAAAGTGAAGGAGAAACTCCAGCTCCTGG | |
| GAACCCCTGGGTCCTGGGGTCACATCACCGAGCAGAGTGGGACCCACGGCTATCTTGGAC | |
| TCAACTCCCAGCAGGTGGAATACCTGGTCAGGAAGAAGCACATCTATATCCCAAAGAACG | |
| GTCAGATTAACTTCAGCTGTATCAATGCCAACAACATAAATTACATCACTGAGGGCATCA | |
| ATGAGGCTGTCCTCCTCACAGAGAGCTCAGAGATGTGTCTTCCAAAGGAAAAAAAAACAC | |
| TGATTGGAATAAAACTTTAG | |

In a search of public sequence databases, the NOV69b nucleic acid sequence, located on chromsome 8 has has 401 of 620 bases (64%) identical to a gb:GENBANK-ID:RATCASPAT|acc:D00252.1 mRNA from Rattus norvegicus (Rattus norvegicus mRNA for cytosolic aspartate aminotransferase, complete cds). Public nucleotide databases include all GenBank databases and the GeneSeq patent database.

The disclosed NOV69b polypeptide (SEQ ID NO:166) encoded by SEQ ID NO:165 has 421 amino acid residues and is presented in Table 69D using the one-letter amino acid code. Signal P, Psort and/or Hydropathy results predict that NOV69b has no signal peptide and is likely to be localized in the cytoplasm with a certainty of 0.3645.

TABLE 69D
Encoded NOV69b protein sequence.

| | |
|---|---|
| MPTLSVFMDVPLAHKLEGSLLKTYKQDDYPNKIFLAYRVCMTNEGHPWVSLVVQKTRLQI | (SEQ ID NO:166) |
| SQDPSLNYEYLPTMGLKSFIQASLALLFGKHSQAIVENRAGGVHTVGDSGAFQLGVQFLR | |
| AWHKDARIVYIISSQKELHGLVFQDMGFTVYEYSVWDPKKLCMDPDILLNVVEQIPHGCV | |
| LVMGNIIDCKLTPSGWAKLMSMIKSKQIFPFFDIPCQGLYTSDLEEDTRILQYFVSQGFE | |
| FFCSQSLSKNFGIYDEGVGMLVVVAVNNQQLLCVLSQLEGLAQALWLNPPNTGARVITSI | |
| LCNPALLGEWKQSLKEVVENIMLTKEKVKEKLQLLGTPGSWGHITEQSGTHGYLGLNSQQ | |
| VEYLVRKKHIYIPKNGQINFSCINANNINYITEGINEAVLLTESSEMCLPKEKKTLIGIK | |
| L | |

A search of sequence databases reveals that the NOV69b amino acid sequence has have 163 of 405 amino acid residues (40%) identical to, and 236 of 405 amino acid residues (58%) similar to, the 412 amino acid residue ptnr:SWISSNEW-ACC:P17174 protein from Homo sapiens (Human) (ASPARTATE AMINOTRANSFERASE, CYTOPLASMIC (EC 2.6.1.1) (TRANSAMINASE A) (GLUTAMATE OXALOACETATE TRANSAMINASE-1)). Public amino acid databases include the GenBank databases, SwissProt, PDB and PIR.

NOV69b is expressed in at least testis. This information was derived by determining the tissue sources of the sequences that were included in the invention including but not limited to SeqCalling sources, Public EST sources, Literature sources, and/or RACE sources.

The disclosed NOV69b polypeptide has homology to the amino acid sequences shown in the BLASTP data listed in Table 69E.

TABLE 69E
BLAST results for NOV69b

| Gene Index/ | | Length | Identity | Positives | |
|---|---|---|---|---|---|
| Identifier | Protein/Organism | (aa) | (%) | (%) | Expect |
| gi\|12840318\|dbj\|BAB24820.1\| | homolog to | 264 | 185/296 | 185/296 | e−102 |
| (AK006984) | ASPARTATE | | (62%) | (62%) | |
| | AMINOTRANSFERASE, | | | | |
| | CYTOPLASMIC (EC | | | | |
| | 2.6.1.1) | | | | |
| | (TRANSAMINASE A) | | | | |
| | (GLUTAMATE | | | | |
| | OXALOACETATE | | | | |
| | TRANSAMINASE- | | | | |
| | 1)˜putative [Mus | | | | |
| | musculus] | | | | |
| gi\|345752\|pir\|\|S29028 | aspartate | 413 | 224/374 | 224/374 | 2e−80 |
| | transaminase (EC | | (59%) | (59%) | |
| | 2.6.1.1) (clone | | | | |
| | 8C7) - human | | | | |
| gi\|91997\|pir\|\|JT0439 | aspartate | 413 | 155/374 | 224/374 | 2e−80 |
| | transaminase (EC | | (41%) | (59%) | |
| | 2.6.1.1), | | | | |
| | cytosolic - rat | | | | |
| >gi\|105387\|pir\|\|S13035 | aspartate | 412 | 155/371 | 222/371 | 3e−80 |
| | transaminase (EC | | (41%) | (59%) | |
| | 2.6.1.1) - human | | | | |
| gi\|6754034\|ref\|NP_034454.1\| | glutamate | 412 | 154/369 | 223/369 | 4e−80 |
| (NM_010324) | oxaloacetate | | (41%) | (59%) | |
| | transaminase 1, | | | | |
| | soluble; | | | | |
| | cytosolic | | | | |
| | aspartate | | | | |
| | aminotransferase | | | | |
| | [Mus musculus] | | | | |

Table 69F-G lists the domain descriptions from DOMAIN analysis results against NOV69b. This indicates that the NOV69b sequence has properties similar to those of other proteins known to contain this domain.

TABLE 69F
Domain Analysis of NOV69b

gnl|Pfam|pfam00873, ACR_tran, AcrB/AcrD/AcrF family.
Members of this family are integral membrane proteins. Some are
involved in drug resistance.
CD-Length = 1020 residues, 15.0% aligned
Score = 51.2 bits (121), Expect = 4e−07

TABLE 69G
Domain Analysis of NOV69b

gnl|Pfam|pfam02460, Patched, Patched family. The
transmembrane protein Patched is a receptor for the morphogene
Sonic Hedgehog. This protein associates with the smoothened
protein to transduce hedgehog signals.
CD-Length = 821 residues, 31.4% aligned
Score = 46.6 bits (109), Expect = 1e−05

Concentrations of glutamate, aspartate and glycine are significantly increased in epileptogenic cerebral cortex. The activities of the enzymes, glutamate dehydrogenase and aspartate aminotransferase, involved in glutamate and aspartate metabolism are also increased.

Polyamine synthesis is enhanced in epileptogenic cortex and may contribute to the activation of N-methyl-D-aspartate (NMDA) Receptors (See Sherwin A L (1999). Neurochem Res 24(11):1387-95). Nuclear magnetic resonance spectroscopy (NMRS) reveals that patients with poorly controlled complex partial seizures have a significant diminution in occipital lobe gamma aminobutyric acid (GABA) concentration. The activity of the enzyme GABA-aminotransaminase (GABA-T) which catalyzes GABA degradation is not altered in epileptogenic cortex. NMRS studies show that vigabatrin, a GABA-T inhibitor and effective antiepileptic, significantly increases brain ABA. Glutamate decarboxylase (GAD), responsible for GABA synthesis, is diminished in interneurons in discrete regions of epileptogenic cortex and hippocampus. In vivo microdialysis performed in epilepsy surgery patients provides measurements of extracellular amino acid levels during spontaneous seizures. Glutamate concentrations are higher in epileptic hippocampi and increase berore seizure onset reaching potentially excitotoxic levels. Frontal or temporal cortical epileptogenic foci also release aspartate, glutamate and serine particularly during intense seizures or status epilepticus. GABA in contrast, exhibits a delayed and feeble rise in the epileptic hippocampus possibly due to a reduction in the number and/or efficiency of GABA transporters. In additon, aspartate aminotransferase activity is an important index for liver function. Abnormal level and activity of aspartate aminotransferase correlates diseased liver conditions, e.g., hepatitis (See Gopal et al., (2000). Postgrad Med 107(2):100-2, 105-9, 113-4; Vesely et al., (1999). Am J Med Sci 317(6):419-24; Johnston D E (1999). Am Fam Phys 59(8):2223-30; Johnston S C, Pelletier L L (1997). Medicine (Baltimore) 76(3):185-91). Finally, aspartate aminotransferase activity is also a marker for diagnosis of cardiovascular diseases (See Wu AH (1999). Ann Clin Lab Sci 29(1):18-23) and periodontal disease (See Eley B M, Cox S W (1998). Br Dent J 184(9):427-30). Therefore, aspartate aminotransferase is an excellent small molecule target and diagnostic marker for epilepsis, liver diseases, cardiovascular and periodontal diseases.

The disclosed NOV69 nucleic acid of the invention encoding a aspartate aminotransferase-like protein includes the nucleic acid whose sequence is provided in Table 69A or 69C or a fragment thereof. The invention also includes a mutant or variant nucleic acid any of whose bases may be changed from the corresponding base shown in Table 69A or 69C while still encoding a protein that maintains its aspartate aminotransferase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36 percent of the bases may be so changed.

The disclosed NOV69 protein of the invention includes the aspartate aminotransferase-like protein whose sequence is provided in Table 69B. The invention also includes a mutant or variant protein any of whose residues may be changed from the corresponding residue shown in Table 69B while still encoding a protein that maintains its aspartate aminotransferase-like activities and physiological functions, or a functional fragment thereof. In the mutant or variant protein, up to about 60 percent of the residues may be so changed.

The invention further encompasses antibodies and antibody fragments, such as F_{ab} or (F_{ab})₂, that bind immunospecifically to any of the proteins of the invention.

The above defined information for this invention suggests that this aspartate aminotransferase-like protein (NOV69) may function as a member of a “aspartate aminotransferase family”. Therefore, the NOV69 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV69 nucleic acids and proteins of the invention are useful in potential therapeutic applications implicated in diseases including but not limited to various pathologies and disorders as indicated below. For example, a cDNA encoding the aspartate aminotransferase-like protein (NOV69) may be useful in gene therapy, and the aspartate aminotransferase-like protein (NOV69) may be useful when administered to a subject in need thereof. By way of nonlimiting example, the compositions of the present invention will have efficacy for treatment of patients suffering from fertility, hypogonadism, or other pathologies or conditions. The NOV69 nucleic acid encoding the aspartate aminotransferase-like protein of the invention, or fragments thereof, may further be useful in diagnostic applications, wherein the presence or amount of the nucleic acid or the protein are to be assessed.

NOV69 nucleic acids and polypeptides are further useful in the generation of antibodies that bind immuno-specifically to the novel NOV69 substances for use in therapeutic or diagnostic methods. These antibodies may be generated according to methods known in the art, using prediction from hydrophobicity charts, as described in the “Anti-NOVX Antibodies” section below. The disclosed NOV69 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV70

A disclosed NOV70 nucleic acid of 4915 nucleotides (also referred to as CG-57462-01) encoding a KIAA1337-like protein is shown in Table 70A. The start and stop codons are in bold letters.

TABLE 70A
NOV70 nucleotide sequence.

| | |
|---|---|
| AATATGCCTGCCATAAAGGGGAACCGGTGGTGGCAGTGGAGCTGGTGCAGGTGTGGAAAG | (SEQ ID NO:167) |
| TCATGAGAATCCTCCTCCTCGGGTTCATCCCCAGGTTTCCTCCCCTTCCCCTGCCCGCGC | |
| CTGCTTGCAGGAAGCGTGTTTACTACAGGGGCCTGGGGATGCTGGCCTCACACATCCCTG | |
| CCCAGCCACAGGGTACTTCCCTGAAGCCTCCTGTACCTTCAGCCCCATCCTCGATTCTCG | |
| CCTCCGGCTCCTCCTCCCCCCACGCCCTCCGGAATGAGCCCCGTACCCCCACCCTCACGC | |
| GCCCTCGCTGTATAAACGCCCTCACTTGTACTGCAAGTCCCTGCGGTCCCACCTTCAGGC | |
| TTCAGCATTCGCTCGACGCATCCCCCAGGCCTGCTTGCCTTGTCACTGTAGCGCCAGACC | |
| CAGCTTCCTTTGCGGCTCCGAGAAGCTTCCCCCTGCGACTTCCGCGAGGAGACGAGTCTG | |
| CGCAGCGTGGTGGCCGCCGCCCCCCGACCCTCTGCGCACTCTCTCCCGCGCCGGCGGCTC | |
| AGCCTAGCCCCGTTCGGCCGGCCGAGACTATGGACACGGAGGATGACCCCTTGCTGCAGG | |
| ATGTGTGGCTAGAGGAGGAGCAGGAGGAGGAAGAAGCAACGGGTGAAACCTTTTTAGGGG | |
| CCCAGAAGCCAGGGCCCCAACCTGGGGCAGGGGGACAGTGTTGCTGGCGGCACTGGCCCC | |
| TGGCTTCCCGACCCCCAGCTTCGGGCTTCTGGAGTACCCTGGGCTGGGCCTTCACCAATC | |
| CGTGCTGTGCTGGGCTGGTGCTCTTCCTGGGCTGCAGCATCCCCATGGCCCTGTCAGCCT | |
| TCATGTTCCTTTACTACCCACCGCTGGACATTGACATCTCCTACAACGCCTTTGAGATCC | |
| GCAACCACGAGGCCTCACAGCGTTTCGACGCTCTCACTCTGGCGCTTAAGTCCCAGTTTG | |
| GATCCTGGGGGCGGAACCGGCGCGATTTGGCCGACTTCACCTCCGAGACGCTTCAGCGCC | |
| TTATCTCAGAGCAGCTGCAGCAGCTGCATCTCGGCAACCGCTCGCGGCAAGCCTCCCGAG | |
| CCCCCCGCGTCATCCCCGCGGCCTCACTCGGTAGCCCAGGCCCTTACCGGGACACTTCCG | |
| CGGCTCAAAAGCCCACAGCCAATCGGAGCGGGCGACTTCGGCGTGAGACCCCGCCCCTGG | |
| AGGATCTGGCAGCCAACCAGAGTGAAGACCCGCGAAACCAGCGGCTGAGCAAGAATGGGC | |
| GGTACCAGCCCAGCATCCCGCCCCACGCGGCAGTCGCGGCCAATCAGAGCCGTGCCCGCC | |
| GAGGCGCCTCGCGCTGGGACTACTCGCGCGCCTATGTGAGTGCCAACACTCAGACGCACG | |
| CGCACTGGCGCATCGAGCTCATCTTCCTGGCGCGCGGCGACGCGGAGCGCAACATTTTCA | |
| CCAGTGAGCGCCTGGTCACGATCCATGAGATCGAGCGCAAGATCATGGACCACCCAGGCT | |
| TCCGGGAGTTCTGCTGGAAGCCCCACGAGGTGCTCAAGGATCTGCCGCTGGGCTCCTACT | |
| CCTACTGCTCGCCCCCCAGCTCGCTCATGACCTACTTTTTTCCCACCGAGAGGGGCGGCA | |
| AGATCTACTATGACGGCATGGGCCAGGACCTGGCGGACATCCGGGGCTCCCTGGAGCTGG | |
| CCATGACTCACCCTGAGTTCTACTGGTATGTGGATGAGGGCCTCTCTGCAGACAATCTGA | |
| AGAGCTCCCTCCTGCGCAGTGAGATCCTGTTTGGAGCACCCCTGCCCAACTACTACTCAG | |
| TAGATGACCGCTGGGAGGAACAACGGGCTAAGTTTCAGAGCTTCGTGGTCACCTACGTGG | |
| CCATGCTGGCCAAGCAGTCTACCAGCAAAGTCCAGGTTCTCTATGGGGGGACAGACCTGT | |
| TTGACTATGAAGTGCGCAGGACGTTCAACAATGACATGCTCCTGGCCTTCATCAGCAGCA | |
| GCTGCATTGCTGCCCTGGTCTACATCCTCACCTCCTGCTCAGTGTTCCTGTCCTTCTTTG | |
| GGATTGCCAGCATTGGTCTCAGCTGCCTGGTGGCCCTCTTCCTGTACCACGTGGTCTTTG | |
| GTATCCAGTACTTGGGCATCCTGAATGGGGTGGCCGCCTTCGTGATCGTGGGCATTGGTG | |
| TGGACGATGTCTTTGTGTTCATCAACACCTACCGCCAGGCCACCCACCTGGAAGACCCAC | |
| AGCTGCGCATGATCCACACCGTCCAAACTGCAGGCAAGGCCACCTTCTTCACCTCCCTGA | |
| CCACAGCCGCCGCCTACGCAGCTAACGTCTTCTCCCAGATCCCAGCCGTCCACGACTTTG | |
| GCCTGTTCATGTCTCTCATCGTGTCCTGTTGCTGGCTGGCCGTGCTTGTCACCATGCCTG | |
| CAGCTCTGGGCCTCTGGAGCCTCTACCTGGCACCACTGGAGAGCTCCTGCCAGACCAGCT | |
| GCCACCAGAATTGCAGCCGGAAGACCTCCCTGCACTTCCCCGGAGACGTGTTTGCCACTC | |
| CCGAGCAGGTTGGAGGCAGCCCTGCCCAGGCCCCCATACCCTACCTGGATGATGACATCC | |
| CCTTGCTGGAGGTCGAGGAAGAGCCAGTGTCACTGGAGCTGGGAGACGTGTCCCTGGTGT | |
| CTGTGTCCCCCGAGGGTCTGCAGCCAGCCTCCAACACGGGCAGCCGCGGCCATCTCATCG | |
| TGCAGCTGCAGGAGCTGCTGCACCACTGGGTCCTGTGGTCAGCCGTCAAGAGCCGCTGGG | |
| TGATTGTGGGGCTGTTCGTCTCCATCCTCATCTTGTCCCTGGTGTTCGCCAGCCGGCTCC | |
| GCCCCGCCAGCCGGGCCCCGCTACTCTTCCGGCCTGATACCAACATCCAGGTGCTGCTGG | |
| ACCTCAAGTACAACCTGAGCGCCGAGGGCATCTCCTGCATCACCTGTTCAGGTCTGTTCC | |
| AGGAGAAGCCCCACAGCCTGCAGAACAACATCCGGACGTCCCTGGAGAAGAAGAGGCGAG | |
| GCTCAGGGGTCCCCTGGGCTAGCCGGCCTGAGGCCACCCTGCAGGATTTCCCAGGCACCG | |
| TGTACATCTCTAAAGTGAAGAGTCAAGGCCACCCCGCTGTCTACAGGCTCTCCCTCAATG | |
| CCAGCCTGCCTGCTCCTTGGCAGGCTGTGTCGCCTGGGGATGGAGAGGTGCCCTCCTTCC | |
| AGGTGTATAGAGCGCCTTTTGGTAACTTCACCAAGAAGCTGACCGCTTGTATGTCTACAG | |
| TAGGGCTGCTCCAGGCGGCGAGCCCCTCCCGCAAGTGGATGCTGACGACCTTGGCCTGTG | |
| ATGCCAAGCGGGGCTGGAAGTTTGACTTCAGCTTCTACGTGGCCACCAAGGAGCAGCAGC | |
| ACACCCGGAAGCTGTACTTCGCCCAGTCCCACAAGCCCCCCTTCCACGGGCGCGTATGCA | |
| TGGCACCCCCTGGCTGCCTGCTTAGCTCCAGCCCCGATGGGCCTACCAAAGGCTTCTTCT | |
| TCGTGCCTAGTGAGAAAGTGCCCAAGGCCCGTCTCTCAGCCACCTTCGGCTTCAACCCCT | |
| GCGTGAACACGGGCTGCGGGAAGCCGGCGGTGCGGCCACTAGTGGATACCGGGGCCATGG | |
| TCTTTGTGGTCTTCGGCATTATTGGCGTCAACCGCACTCGGCAGGTGGACAACCACGTCA | |
| TTGGAGACCCGGGTAGTGTTGTCTACGACAGCAGCTTTGACCTCTTCAAGGAAATTGGGC | |
| ACCTGTGTCACCTCTGCAAGGCCATCGCAGCCAACTCCGAGCTGGTGAAGCCGGGTGGGG | |
| CCCAGTGCCTGCCTTCAGGCTACAGCATCTCCTCCTTCCTGCAGATGTTGCACCCTGAGT | |
| GCAAGGAGCTGCCCGAGCCCAACCTGCTCCCGGGGCAGCTGTCCCACGGGGCAGTGGGCG | |
| TCAGGGAGGGCCGCGTGCAGGAGATCTCCATGGCTTTCGAGTCGACCACGTACAAGGGCA | |
| AATCCTCCTTCCAGACCTACTCGGACTACCTGCGCTGGGAGAGCTTCCTCCAGCAGCAGC | |
| TGCAGGCCTTGCCCGAGGGCTCAGTCCTGCGCCGCGGCTTCCAGACCTGCGAGCACTGGA | |
| AGCAGATATTCATGGAAATCGTAGGGGTGCAGAGCGCCCTGTGCGGCCTGGTGCTATCCC | |
| TGCTCATCTGCGTGGCCGCGGTGGCCGTGTTCACCACCCACATCCTGCTCCTGCTGCCCG | |
| TGCTCCTCAGCATCTTGGGCATCGTGTGCCTGGTGGTGACCATCATGTACTGGAGCGGCT | |
| GGGAGATGGGGGCTGTGGAAGCCATCTCCCTGTCCATCCTCGTTGGCTCCTCCGTGGATT | |
| ACTGCGTCCACCTGGTCGAGGGCTACCTGCTGGCTGGAGAGAACCTGCCCCCCCACCAGG | |
| CCGAGGACGCCCGAACGCAGCGCCAGTGGCGTACGCTGGAGGCCGTGCGGCACGTGGGCG | |
| TGGCCATCGTCTCCAGTGCCCTCACCACGGTCATCGCCACAGTGCCCCTCTTCTTCTGCA | |
| TCATCGCCCCATTTGCCAAGTTCGGCAAGATTGTGGCACTCAACACGGGCGTGTCCATCC | |
| TCTACACGCTGACCGTCAGCACCGCCCTGCTGGGCATCATGGCGCCCAGCTCTTTCACTC | |
| GGACCCGGACTTCCTTCCTCAAGGCCCTGGGTGCCGTGCTGCTGGCAGGGGCCCTGGGGC | |
| TGGGTGCCTGCCTCGTGCTCCTGCAGAGCGGCTATAAGATTCCCCTGCCCGCAGGGGCCT | |
| CCCTATAGCCCGGGACGGGCTCTGGACACTTGCACCTTTGGTCCCATGGGTGGGGGACAG | |
| GAGCTGCTTCCCAGCTCGACTTCAGCTAGCTGTGTCCCCAGGCCTGGGCCCAGGGCGCCC | |
| TGCGGGCCAGCGTGGAGGCTGACACCCACACAGATGGTGTGGACCATGCTGCCTT | |

In a search of public sequence databases, the NOV70 nucleic acid sequence, located on chromsome 1 has 4481 of 4484 bases (99%) identical to a gb:GENBANK-ID:AB037758|acc:AB037758.1 mRNA from Homo sapiens (Homo sapiens mRNA for KIAA1337 protein, partial cds). Public nucleotide databases include all GenBank databases and the GeneSeq patent database.

The disclosed NOV70 polypeptide (SEQ ID NO:168) encoded by SEQ ID NO:167 has 1561 amino acid residues and is presented in Table 70B using the one-letter amino acid code. Signal P, Psort and/or Hydropathy results predict that NOV70 has a signal peptide and is likely to be localized plasma membrane with a certainty of 0.8000. The most likely cleavage site for a NOV70 peptide is between amino acids 18 and 19.

TABLE B
Encoded NOV70 protein sequence.

| | |
|---|---|
| MRILLLGFIPRFPPLPLPAPACRKRVYYRGLGMLASHIPAQPQGTSLKPPVPSAPSSILA | (SEQ ID NO:168) |
| SGSSSPHALRNEPRTPTLTRPRCINALTCTASPCGPTFRLQHSLDASPRPACLVTVAPDP | |
| ASFAAPRSFPLRLPRGDESAQRGGRRPPTLCALSPAPAAQPSPVRPAETMDTEDDPLLQD | |
| VWLEEEQEEEEATGETFLGAQKPGPQPGAGGQCCWRHWPLASRPPASGFWSTLGWAFTNP | |
| CCAGLVLFLGCSIPMALSAFMFLYYPPLDIDISYNAFEIRNHEASQRFDALTLALKSQFG | |
| SWGRNRRDLADFTSETLQRLISEQLQQLHLGNRSRQASRAPRVIPAASLGSPGPYRDTSA | |
| AQKPTANRSGRLRRETPPLEDLAANQSEDPRNQRLSKNGRYQPSIPPHAAVAANQSRARR | |
| GASRWDYSRAYVSANTQTHAHWRIELIFLARGDAERNIFTSERLVTIHEIERKIMDHPGF | |
| REFCWKPHEVLKDLPLGSYSYCSPPSSLMTYFFPTERGGKIYYDGMGQDLADIRGSLELA | |
| MTHPEFYWYVDEGLSADNLKSSLLRSEILFGAPLPNYYSVDDRWEEQRAKFQSFVVTYVA | |
| MLAKQSTSKVQVLYGGTDLFDYEVRRTFNNDMLLAFISSSCIAALVYILTSCSVFLSFFG | |
| IASIGLSCLVALFLYHVVFGIQYLGILNGVAAFVIVGIGVDDVFVFINTYRQATHLEDPQ | |
| LRMIHTVQTAGKATFFTSLTTAAAYAANVFSQIPAVHDFGLFMSLIVSCCWLAVLVTMPA | |
| ALGLWSLYLAPLESSCQTSCHQNCSRKTSLHFPGDVFATPEQVGGSPAQAPIPYLDDDIP | |
| LLEVEEEPVSLELGDVSLVSVSPEGLQPASNTGSRCHLIVQLQELLHHWVLWSAVKSRWV | |
| IVGLFVSILILSLVFASRLRPASRAPLLFRPDTNIQVLLDLKYNLSAEGISCITCSGLFQ | |
| EKPHSLQNNIRTSLEKKRRGSGVPWASRPEATLQDFPGTVYISKVKSQGHPAVYRLSLNA | |
| SLPAPWQAVSPGDGEVPSFQVYRAPFGNFTKKLTACMSTVGLLQAASPSRKWMLTTLACD | |
| AKRGWKFDFSFYVATKEQQHTRKLYFAQSHKPPFHGRVCMAPPGCLLSSSPDGPTKGFFF | |
| VPSEKVPKARLSATFGFNPCVNTGCGKPAVRPLVDTGAMVFVVFGIIGVNRTRQVDNHVI | |
| GDPGSVVYDSSFDLFKEIGHLCHLCKAIAANSELVKPGGAQCLPSGYSISSFLQMLHPEC | |
| KELPEPNLLPGQLSHGAVGVREGRVQEISMAFESTTYKGKSSFQTYSDYLRWESFLQQQL | |
| QALPEGSVLRRGFQTCEHWKQIFMEIVGVQSALCGLVLSLLICVAAVAVFTTHILLLLPV | |
| LLSILGIVCLVVTIMYWSGWEMGAVEAISLSILVGSSVDYCVHLVEGYLLAGENLPPHQA | |
| EDARTQRQWRTLEAVRHVGVAIVSSALTTVIATVPLFFCIIAPFAKFGKIVALNTGVSIL | |
| YTLTVSTALLGIMAPSSFTRTRTSFLKALGAVLLAGALGLGACLVLLQSGYKIPLPAGAS | |
| L | |

A search of sequence databases reveals that the NOV70 amino acid sequence has 1436 of 1438 amino acid residues (99%) identical to, and 1436 of 1438 amino acid residues (99%) similar to, the 1438 amino acid residue ptnr:SPTREMBL-ACC:Q9P2K9 protein from Homo sapiens (Human) (KIAA1337 PROTEIN). Public amino acid databases include the GenBank databases, SwissProt, PDB and PIR.

NOV70 is expressed in at least Brain, Cerebral Medulla/Cerebral white matter, hippocampus, Hypothalamus, Left cerebellum, Lung, Parietal Lobe, Testis, and Right Cerebellum. This information was derived by determining the tissue sources of the sequences that were included in the invention including but not limited to SeqCalling sources, Public EST sources, Literature sources, and/or RACE sources.

The disclosed NOV70 polypeptide has homology to the amino acid sequences shown in the BLASTP data listed in Table 70C.

TABLE 70C
BLAST results for NOV70

| Gene Index/ | | Length | Identity | Positives | |
|---|---|---|---|---|---|
| Identifier | Protein/Organism | (aa) | (%) | (%) | Expect |
| gi\|7243055\|dbj\|BAA92575.1\| | KIAA1337 protein | 1438 | 1436/1438 | 1436/1438 | 0.0 |
| (AB037758) | [Homo sapiens] | | (99%) | (99%) | |
| gi\|17448847\|ref\|XP_ | KIAA1337 protein | 1392 | 1388/1392 | 1388/1392 | 0.0 |
| 052561.4\| | [Homo sapiens] | | (99%) | (99%) | |
| (XM_052561) | | | | | |
| gi\|5834578\|emb\|CAB55303.1\| | hypothetical | 594 | 592/594 | 592/594 | 0.0 |
| (AL117236) | protein [Homo | | (99%) | (99%) | |
| | sapiens] | | | | |
| gi\|17448809\|ref\|XP_ | Similar to | 383 | 361/363 | 361/363 | 0.0 |
| 052559.3\| | KIAA1337 protein | | (99%) | (99%) | |
| (XM_052559) | [Homo sapiens] | | | | |
| gi\|18545186\|ref\|XP_ | protein MGC13130 | 1524 | 54/177 | 89/177 | 5e−14 |
| 046122.2\| | [Homo sapiens] | | (30%) | (49%) | |
| (XM_046122) | | | | | |

Tables 70D-E list the domain descriptions from DOMAIN analysis results against NOV70. This indicates that the NOV70 sequence has properties similar to those of other proteins known to contain this domain.

TABLE 70D
Domain Analysis of NOV70

gnl|Pfam|pfam00873, ACR_tran, AcrB/AcrD/AcrF family.
Members of this family are integral membrane proteins. Some are
involved in drug resistance.
CD-Length = 1020 residues, 15.0% aligned
Score = 51.2 bits (121), Expect = 4e−07

TABLE 70E
Domain Analysis of NOV70

gnl|Pfam|pfam02460, Patched, Patched family. The
transmembrane protein Patched is a receptor for the morphogene
Sonic Hedgehog. This protein associates with the smoothened
protein to transduce hedgehog signals.
CD-Length = 821 residues, 31.4% aligned
Score = 46.6 bits (109), Expect = 1e−05

The Drosophila ‘patched’ (ptc) gene encodes a transmembrane protein that represses transcription in specific cells of genes encoding members of the TGF beta (see 190180) and Wnt (164820) families of signaling proteins. Vertebrate homologs of ptc have been identified in mice, chickens, and zebra fish. The human PTC protein is predicted to contain 12 hydrophobic membrane-spanning domains and 2 large hydrophilic extracellular loops. Johnson et al. (1996) mapped the human PTC gene to chromosome 9q22.3 by radiation hybrid analysis.

The murine PTCH homolog, Ptc, maps to mouse chromosome 13. Ptc maps close to the murine Face locus (0 recombinants in 188 meioses). They noted that mouse mutations such as flexed tail (f), purkinje cell degeneration (pcd), and mesenchymal dysplasia (mes) which involve abnormal development of skeletal and neural tissues are also located in this region of chromosome 13 and may be allelic to Ptc. The 2 extracellular loops of the Ptc protein are necessary for binding and that binding also requires that the Ptc protein be glycosylated. Marigo et al. (1996) proposed that Ptc does not carry out signaling to the cell directly but that an additional molecule is involved, namely the 7-transmembrane protein ‘Smoothened’ (SMO; 601500). The Ptc gene encodes a candidate receptor for Shh by showing that epitope-tagged N-Shh binds specifically to human embryonic kidney 293 cells expressing mouse Ptc.

Basal cell carcinoma, medulloblastoma, rhabdomyosarcoma, and other human tumors are associated with mutations that activate the protooncogene ‘Smoothened’ or that inactivate the tumor suppressor ‘Patched.’ Smoothened and Patched mediate the cellular response to the Hedgehog secreted protein signal, and oncogenic mutations affecting these proteins cause excess activity of the Hedgehog response pathway.

Approximately 5% of patients with Gorlin syndrome develop medulloblastoma in the first few years of life, and 10% of patients with medulloblastoma diagnosed at age 2 years or under have Gorlin syndrome. Cowan et al. (1997) found that 1 out of 3 unrelated patients with medulloblastoma complicated by Gorlin syndrome had lost the wildtype allele on 9q, indicating that the Gorlin locus probably acts a tumor suppressor in the development of this tumor. They also confirmed this role in a basal cell carcinoma from the same individual. Studying patients who presented with multiple odontogenic keratocysts, Lench et al. (1997) identified 5 novel germline mutations in PTCH. Four mutations caused premature stop codons and 1 resulted in an amino acid substitution toward the C terminus of the predicted protein.

The disclosed NOV70 nucleic acid of the invention encoding a KIAA1337-like protein includes the nucleic acid whose sequence is provided in Table 70A or a fragment thereof. The invention also includes a mutant or variant nucleic acid any of whose bases may be changed from the corresponding base shown in Table 70A while still encoding a protein that maintains its KIAA1337-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV70 protein of the invention includes the KIAA1337-like protein whose sequence is provided in Table 70B. The invention also includes a mutant or variant protein any of whose residues may be changed from the corresponding residue shown in Table 70B while still encoding a protein that maintains its KIAA1337-like activities and physiological functions, or a functional fragment thereof. In the mutant or variant protein, up to about 1 percent of the residues may be so changed.

The invention further encompasses antibodies and antibody fragments, such as F_{ab} or (F_{ab})₂, that bind immunospecifically to any of the proteins of the invention.

The above defined information for this invention suggests that this KIAA1337-like protein (NOV70) may function as a member of a “KIAA1337 family”. Therefore, the NOV70 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV70 nucleic acids and proteins of the invention are useful in potential therapeutic applications implicated in diseases including but not limited to various pathologies and disorders as indicated below. For example, a cDNA encoding the KIAA1337-like protein (NOV70) may be useful in gene therapy, and the KIAA1337-like protein (NOV70) may be useful when administered to a subject in need thereof. By way of nonlimiting example, the compositions of the present invention will have efficacy for treatment of patients suffering from Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Endocrine dysfunctions, Diabetes, obesity, Growth, Systemic lupus erythematosus, Autoimmune disease, Asthma, Emphysema, Scleroderma, allergy, Fertility, ARDS, Pharyngitis, Laryngitis, Myasthenia gravis, or other pathologies or conditions. The NOV70 nucleic acid encoding the KIAA1337-like protein of the invention, or fragments thereof, may further be useful in diagnostic applications, wherein the presence or amount of the nucleic acid or the protein are to be assessed.

NOV70 nucleic acids and polypeptides are further useful in the generation of antibodies that bind immuno-specifically to the novel NOV70 substances for use in therapeutic or diagnostic methods. These antibodies may be generated according to methods known in the art, using prediction from hydrophobicity charts, as described in the “Anti-NOVX Antibodies” section below. The disclosed NOV70 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV71

A disclosed NOV71 nucleic acid of 2004 nucleotides (also referred to as CG57584-01) encoding a zona pellucida glycoprotein 1 precursor-like protein is shown in Table 71A. The start and stop codons are in bold letters.

TABLE 71A
NOV71 nucleotide sequence.

| | |
|---|---|
| CACCCACTTGCCACCTCCTCCATAAAAGGCCTGGGCCCAGCTCTGGTGGCGAGGGAGTAG | (SEQ ID NO:169) |
| GGGGTGTGTCTGTGGCGTCTCATGGCAGGAGGCTCAGCCACGACCTGGGGTTACCCTGTG | |
| GCCCTGCTACTGCTGGTCGCCACCCTGGGGCTGGGTAGCTACGACTGTGGGATCAAGGGA | |
| ATGCAGCTGCTGGTGTTCCCCAGGCCAGGCCAGACTCTCCCCTTCAAGGTGGTGGATGAA | |
| TTTGGGAACCGATTTGATGTCAACAACTGCTCCATCTGCTACCACTGGGTCACCTCCAGG | |
| CCGCAGGAGCCTGCAGTCTTCTCGGCCGATTACAGAGGCTGCCACGTGCTGGAGAAGGAT | |
| GGGCGTTTCCACCTGAGGGTGTTCATGGAGGCTGTGCTGCCCAATGGTCGTGTGGATGTG | |
| GCACAAGACGCTACTCTGATCTGTCCCAAACCTGACCCCTCCCGGACTCTGGACTCCCAG | |
| CTGGCACCACCCGCCATGTTCTCTGTCTCAACCCCACAAACCCTTTCCTTCCTCCCCACC | |
| TCTGGCCATACCTCCCAAGGCTCTGGCCATGCCTTTCCCAGCCCACTGGACCCAGGGCAC | |
| AGCTCTGTCCACCCAACCCCTGCTTTACCATCCCCTGGACCTGGACCTACCCTCGCCACC | |
| CTGGCTCAACCCCACTGGGGCACCTTGGAACACTGGGATGTGAACAAACGAGATTACATA | |
| GGTACCCACCTGAGCCAGGAGCAGTGCCAGGTGGCCTCAGGGCACCTCCCCTGCATCGTG | |
| AGAAGAACTTCAAAAGAAGCCTGTCAGCAGGCTGGCTGCTGCTATGACAACACCAGAGAG | |
| GTTCCCTGTTACTATGGCAACACAGCTACTGTCCAGTGCTTCAGAGATGGCTACTTCGTC | |
| CTCGTAGTGTCCCAAGAAATGGCCTTGACACACAGGATCACACTGGCCAACATCCACCTG | |
| GCCTATGCCCCCACCAGCTGCTCCCCAACACAGCACACGGAAGCTTTCGTGGTCTTCTAC | |
| TTCCCTCTCACCCACTGTGGAACCACAATGCAGGTGGCTGGCGACCAGCTCATCTATGAG | |
| AACTGGCTGGTGTCTGGCATCCACATCCAAAAGGGGCCACAGGGTTCCATCACGCGGGAC | |
| AGCACCTTCCAGCTTCATGTGCGCTGTGTCTTCAACGCCAGTGACTTCCTGCCCATTCAG | |
| GCATCCATTTTCCCACCCCCATCGCCTGCTCCTATGACCCAGCCCGGCCCCCTGCGGCTT | |
| GAGCTGCGGATTGCCAAAGACGAGACCTGCAGCTCGTACTATGGGGAGGATGACTATCCC | |
| ATCGTGAGGCTGCTCCGAGAACCAGTCCATGTGGAGGTCCGGCTTCTGCAGAGGACAGAC | |
| CCCAACCTGGTCCTGCTGCTGCACCAGTGCTGGGGCGCTCCCAGTGCCAACCCCTTCCAG | |
| CAGCCCCAGTGGCCCATCCTGTCAGACGGATGCCCCTTCAAGGGCGACAGCTACAGAACG | |
| CAAATGGTAGCCTTGGACGGGGCCACACCTTTCCAGTCGCACTACCAGCGATTCACTGTT | |
| GCTACCTTCGCCCTCCTGGACTCAGGCTCCCAGAGAGCCCTCAGAGGACTGGTTTACTTG | |
| TTCTGCAGCACCTCTGCCTGCCACACCTCAGGGCTGGAGACTTGCTCCACTGCATGTAGC | |
| ACTGGCACTACAAGACAGCGACGATCCTCAGGTCACCGTAATGACACTGCCAGGCCCCAG | |
| GACATCGTGAGCTCTCCGGGGCCAGTGGGCTTTGAGGATTCTTATGGGCAGGAGCCCACA | |
| CTTGGGCCCACAGACTCCAATGGGAACTCCAGCCTGAGACCTCTCCTTTGGGCGGTCCTT | |
| TTGCTGCCAGCTGTTGCCCTGGTCCTTGGGTTTGGTGTCTTTGTGGGCCTGAGCCAGACC | |
| TGGGCCCAGAAGCTCTGGGAAGCAACAGACACTGAATGGGCCCAATAAACAATCATTC | |
| CAACCTACTGAAAAAAAAAAAAAA | |

In a search of public sequence databases, the NOV71 nucleic acid sequence, located on chromsome 11 has 1305 of 1704 bases (76%) identical to a gb:GENBANK-ID:MOZP1|acc:U20448.1 mRNA from Mus musculus (Mus musculus ZP1 precursor (Zp-1) mRNA, complete cds). Public nucleotide databases include all GenBank databases and the GeneSeq patent database.

The disclosed NOV71 polypeptide (SEQ ID NO:170) encoded by SEQ ID NO:169 has 624 amino acid residues and is presented in Table 71B using the one-letter amino acid code. Signal P, Psort and/or Hydropathy results predict that NOV71 has a signal peptide and is likely to be localized localized at the plasma membrane with a certainty of 0.4600. The most likely cleavage site for a NOV71 peptide is between amino acids 25 and 26.

TABLE 71B
Encoded NOV71 protein sequence.

| | |
|---|---|
| MAGGSATTWGYPVALLLLVATLGLGSYDCGIKGMQLLVFPRPGQTLPFKVVDEFGNRFDV | (SEQ ID NO:170) |
| NNCSICYHWVTSRPQEPAVFSADYRGCHVLEKDGRFHLRVFMEAVLPNGRVDVAQDATLI | |
| CPKPDPSRTLDSQLAPPAMFSVSTPQTLSFLPTSGHTSQGSGHAFPSPLDPGHSSVHPTP | |
| ALPSPGPGPTLATLAQPHWGTLEHWDVNKRDYIGTHLSQEQCQVASGHLPCIVRRTSKEA | |
| CQQAGCCYDNTREVPCYYGNTATVQCFRDGYFVLVVSQEMALTHRITLANIHLAYAPTSC | |
| SPTQHTEAFVVFYFPLTHCGTTMQVAGDQLIYENWLVSGIHIQKGPQGSITRDSTFQLHV | |
| RCVFNASDFLPIQASIFPPPSPAPMTQPGPLRLELRIAKDETCSSYYGEDDYPIVRLLRE | |
| PVHVEVRLLQRTDPNLVLLLHQCWGAPSANPFQQPQWPILSDGCPFKGDSYRTQWVALDG | |
| ATPFQSHYQRFTVATFALLDSGSQRALRGLVYLFCSTSACHTSGLETCSTACSTGTTRQR | |
| RSSGHRNDTARPQDIVSSPGPVGFEDSYGQEPTLGPTDSNGNSSLRPLLWAVLLLPAVAL | |
| VLGFGVFVGLSQTWAQKLWESNRQ | |

A search of sequence databases reveals that the NOV71 amino acid sequence has 422 of 620 amino acid residues (68%) identical to, and 477 of 620 amino acid residues (76%) similar to, the 623 amino acid residue ptnr:SPTREMBL-ACC:Q62016 protein from Mus musculus (Mouse) (ZONA PELLUCIDA GLYCOPROTEIN 1 PRECURSOR (ZP1)). Public amino acid databases include the GenBank databases, SwissProt, PDB and PIR.

NOV71 is expressed in at least Kidney, Pituitary Gland, Testis, Whole Organism. Expression information was derived from the tissue sources of the sequences that were included in the derivation of the sequence of CG57584-01. The sequence is predicted to be expressed in the following tissues because of the expression pattern of (GENBANK-ID: gb:GENBANK-ID:MOZP1|acc:U20448.1) a closely related Mus musculus ZP1 precursor (Zp-1) mRNA, complete cds homolog in species Mus musculus ovary. This information was derived by determining the tissue sources of the sequences that were included in the invention including but not limited to SeqCalling sources, Public EST sources, Literature sources, and/or RACE sources.

The disclosed NOV71 polypeptide has homology to the amino acid sequences shown in the BLASTP data listed in Table 71C.

TABLE 71C
BLAST results for NOV71

| Gene Index/ | | Length | Identity | Positives | |
|---|---|---|---|---|---|
| Identifier | Protein/Organism | (aa) | (%) | (%) | Expect |
| gi\|17460872\|ref\|XP_ | similar to zona | 496 | 458/458 | 458/458 | 0.0 |
| 062198.1\| | pellucida | | (100%) | (100%) | |
| (XM_062198) | glycoprotein 1 | | | | |
| | (H. sapiens) | | | | |
| gi\|6677653\|ref\|NP_033606.1\| | zona pellucida | 623 | 416/628 | 471/628 | 0.0 |
| (NM_009580) | glycoprotein 1 | | (66%) | (74%) | |
| | [Mus musculus] | | | | |
| gi\|1113794\|gb\|AAB60507.1\| | zona pellucida | 623 | 414/628 | 470/628 | 0.0 |
| (U24230) | [Mus musculus] | | (65%) | (73%) | |
| gi\|16758240\|ref\|NP_ | zona pellucida | 617 | 416/621 | 469/621 | 0.0 |
| 445961.1\| | glycoprotein 1 | | (66%) | (74%) | |
| (NM_053509 | [Rattus | | | | |
| | norvegicus] | | | | |
| gi\|11140012\|emb\|CAC16087.1\| | zona | 934 | 197/369 | 254/369 | 2E−97 |
| (AJ289697) | pellucida protein 1 | | (53%) | (68%) | |
| | [Gallus gallus] | | | | |

Tables 71D-E list the domain descriptions from DOMAIN analysis results against NOV71. This indicates that the NOV71 sequence has properties similar to those of other proteins known to contain this domain.

TABLE 71D
Domain Analysis of NOV71

gnl|Pfam|pfam00100, zona_pellucida, Zona pellucida-like
domain.
CD-Length = 266 residues, 99.6% aligned
Score = 209 bits (533), Expect = 3e−55

TABLE 71E
Domain Analysis of NOV71

gnl|Smart|smart00018, P, P or trefoil or TFF domain;
Proposed role in renewal and pathology of mucous epithelia
CD-Length = 47 residues, 100.0% aligned
Score = 39.7 bits (91), Expect = 5e−04

The mammalian zona pellucida is composed of 3 major glycoproteins, ZP1, ZP2 (182888), and ZP3 (182889). ZP3, the molecule responsible for the major sperm-receptor activity of the zona, plays a significant role in fertilization. ZP2 is implicated as a secondary sperm receptor that binds sperm only after the induction of the sperm acrosome reaction. See review by Dean (1992). The mature ZP1, ZP2, and ZP3 proteins have molecular weights of 90-110 kD, 64-76 kD, and 57-73 kD, respectively.

The disclosed NOV71 nucleic acid of the invention encoding a zona pellucida glycoprotein 1 precursor-like protein includes the nucleic acid whose sequence is provided in Table 71A or a fragment thereof. The invention also includes a mutant or variant nucleic acid any of whose bases may be changed from the corresponding base shown in Table 71A while still encoding a protein that maintains its zona pellucida glycoprotein 1 precursor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 24 percent of the bases may be so changed.

The disclosed NOV71 protein of the invention includes the zona pellucida glycoprotein 1 precursor-like protein whose sequence is provided in Table 71B. The invention also includes a mutant or variant protein any of whose residues may be changed from the corresponding residue shown in Table 71B while still encoding a protein that maintains its zona pellucida glycoprotein 1 precursor-like activities and physiological functions, or a functional fragment thereof. In, the mutant or variant protein, up to about 32 percent of the residues may be so changed.

The invention further encompasses antibodies and antibody fragments, such as F_{ab} or (F_{ab})₂, that bind immunospecifically to any of the proteins of the invention.

The above defined information for this invention suggests that this zona pellucida glycoprotein 1 precursor-like protein (NOV71) may function as a member of a “zona pellucida glycoprotein 1 precursor family”. Therefore, the NOV71 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV71 nucleic acids and proteins of the invention are useful in potential therapeutic applications implicated in diseases including but not limited to various pathologies and disorders as indicated below. For example, a cDNA encoding the zona pellucida glycoprotein 1 precursor-like protein (NOV71) may be useful in gene therapy, and the zona pellucida glycoprotein 1 precursor-like protein (NOV71) may be useful when administered to a subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Aneurysm, Fibromuscular dysplasia, Stroke, Anemia, Bleeding disorders, Adrenoleukodystrophy, Congenital Adrenal Hyperplasia, Diabetes, Von Hippel-Lindau (VHL) syndrome, Pancreatitis, Hyperparathyroidism, Hypoparathyroidism, SIDS, Endometriosis, Fertility, Xerostomia, Hypercalceimia, Ulcers, Cirrhosis, Inflammatory bowel disease, Diverticular disease, Hirschsprung's disease, Crohn's Disease, Appendicitis, Hemophilia, hypercoagulation, Idiopathic thrombocytopenic purpura, autoimmune disease, allergies, immunodeficiencies, Graft vesus host, Ataxia-telangiectasia, Hemophilia, Lymphedema, Tonsilitis, Osteoporosis, Arthritis, Ankylosing spondylitis, Scoliosis, Tendinitis, Muscular dystrophy, Lesch-Nyhan syndrome, Myasthenia gravis, Dental disease and infection, Alzheimer's disease, Tuberous sclerosis, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Growth and reproductive disorders, Endocrine dysfunctions, Systemic lupus erythematosus, Asthma, Emphysema, ARDS, Pharyngitis, Laryngitis, Hearing loss, Tinnitus, Psoriasis, Actinic keratosis, Tuberous sclerosis, Acne, Hair growth, allopecia, pigmentation disorders, cystitis, incontinence, Renal artery stenosis, Interstitial nephritis, Glomerulonephritis, Polycystic kidney disease, Systemic lupus erythematosus, Renal tubular acidosis, IgA nephropathy, Vesicoureteral reflux, glaucoma, blindness, and Hypothyroidism, or other pathologies or conditions. The NOV71 nucleic acid encoding the zona pellucida glycoprotein 1 precursor-like protein of the invention, or fragments thereof, may further be useful in diagnostic applications, wherein the presence or amount of the nucleic acid or the protein are to be assessed.

NOV71 nucleic acids and polypeptides are further useful in the generation of antibodies that bind immuno-specifically to the novel NOV71 substances for use in therapeutic or diagnostic methods. These antibodies may be generated according to methods known in the art, using prediction from hydrophobicity charts, as described in the “Anti-NOVX Antibodies” section below. The disclosed NOV71 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV72

A disclosed NOV72 nucleic acid of 6108 nucleotides (also referred to as CG56761-01) encoding an ankyrin repeat containing protein-like protein is shown in Table 72A. The start and stop codons are in bold letters.

TABLE 72A
NOV72 nucleotide sequence.

| | |
|---|---|
| ATGGCCAGTCACGTGGACCTGCTGACGGAGCTGCAGCTGCTGGAGAAGGTGCCCACGCTG | (SEQ ID NO:171) |
| GAGCGGCTGCGGGCTGCCCAGAAGCGCCGGGCCCAGCAGCTGAAGAAATGGGCACAGTAC | |
| GAGCAGGACTTGCAGCACCGCAAGCGAAAGCATGAGCGGAAGCGCAGCACGGGCGGCCGC | |
| CGCAAGAAAGTGTCCTTCGAGGCCAGCGTGGCCCTGCTGGAGGCCTCGCTGAGGAACGAC | |
| GCCGAGGAAGTACGCTACTTCCTGAAGAATAAGGTCAGCCCTGATTTGTGCAATGAGGAC | |
| GGACTCACAGCCCTACACCAGTGCTGCATCGACAACTTTGAGGAAATTGTGAAGCTGCTC | |
| CTCTCCCATGGTGCCAATGTGAACGCCAAGGACAACGAGCTGTGGACACCTCTCCATGCT | |
| GCAGCCACCTGCGGCCACATCAACCTGGTGAAGATCCTCGTTCAGTATGGGGCCGACTGG | |
| CTTGCTGTCAACTCGGATGGGAACATGCCATATGACCTCTGCGAGGATGAACCCACCCTG | |
| GATGTCATCGAGACCTGCATGGCATACCAGGGCATCACCCAAGAGAAAATCAACGAGATG | |
| CGGGTGGCTCCTGAGCAGCAGATGATTGCGGACATCCACTGCATGATCGCAGCGGGCCAG | |
| GACCTGGACTGGATAGATGCCCAGGGTGCCACACTGCTGCACATAGCTGGAGCCAATGGA | |
| TACCTGCGGGCAGCTGAGCTCCTCCTGGATCATGGAGTGCGTGTGGATGTGAAGGACTGG | |
| GATGGCTGGGAGCCCCTGCATGCAGCTGCCTTCTGGGGACAGATGCAGATGGCAGAGCTA | |
| TTGGTGTCCCATGGAGCTAGTCTCAGTGCAAGGACATCCATGGATGAGATGCCAATAGAC | |
| CTGTGCGAGGAGGAAGAGTTCAAGGTCCTGCTGCTGGAGCTAAAACACAAGCATGATGTG | |
| ATCATGAAGTCACAGCTGAGGCACAAGTCATCCTTGAGCCGGAGGACCTCCAGCGCAGGC | |
| AGCCGTGGGAAGGTGGTGCGGCGAGCCAGCCTGTCGGACAGGACCAACCTGTATAGGAAG | |
| GAGTATGAGGGAGAGGCCATCCTGTGGCAGCGGAGTGCAGCTGAGGATCAGCGGACCTCC | |
| ACCTACAACGGGGACATCAGGGAGACCAGGACAGACCAAGAGAATAAGGACCCTAACCCC | |
| AGGCTGGAGAAGCCCGTGCTACTCTCCGAATTTCCTACCAAGATCCCACGAGGTGAACTG | |
| GACATGCCTGTTGAGAATGGCCTCCGGGCTCCGGTCAGTGCCTACCAGTATGCGCTGGCC | |
| AACGGGGATGTCTGGAAGGTGCATGAGGTGCCTGACTACAGCATGGCCTATGGCAACCCT | |
| GGCGTGGCCGACGCCACCCCGCCCTGGAGCAGCTACAAGGAACAGAGCCCTCAGACGCTT | |
| CTGGAGCTGAAGCGGCAGCGGGCTGCAGCCAAGCTGCTCAGCCACCCCTTCCTTAGCACA | |
| CACCTGGGCAGCAGCATGGCCAGGACGGGCGAGAGTAGCAGTGAAGGCAAGGCCCCCTTG | |
| ATCGGAGGCAGAACTTCACCGTACAGCAGCAATGGGACCTCGGTATATTACACGGTCACC | |
| AGCGGAGATCCCCCACTCTTAAAGTTCAAGGCCCCCATAGAGGAGATGGAGGAGAAGGTG | |
| CATGGCTGTTGCCGTATCTCCTAGTCTCCGTGTGATGGAGGAGGGAGATGCCTGGGGAGG | |
| GGCTCCTGGAATCCAGGCCAGCCCAACAGCCCTGGCTGGGGAGGTGTCAGGGCAGCTGGG | |
| GAGAGGTGGGCTCTGCTTTTCAGAGGAACTCAGACCCCAGCCCTCAGCTGGCTGCCCATA | |
| GCATCCCATGTCCCACGTCCCGTGGTTCTGCTTCCTGCTGCATCGTCTGCCATCTGACAC | |
| AAGGCCTGTCGTGGCCTCCTGGTTCACTCTGCTGTCTGATCTTGGGAGGGTGGGCTTGAG | |
| ATCCCAGCTCTATTCTTGGTATAAAGGCTTCTCCGGATCAGTACATGCATGTCACATTAA | |
| CACACACACACACACACATATACACACACACACAAGCTCGATCAGTGTGTGTAGGAATGA | |
| CATACCTGGGCTCAGGGGAAGCAAGGGGGCTTAGAATTTGTGGGGTATTCCCAAAAGGAT | |
| GGAAGTTAAGACTCAGAGTCTCATTACCACTGCCAATGTGGTTTTAGCAGGGGAGGGGAC | |
| CTGCTAAGCTGAGACCCATAGTCCTGCTCAGAGTTATCCCAAAGTCTGAGCCACCAGCCA | |
| CACCTGACAGGGGTGAGAAGTCCTCGCTGTGTTCAGAGGGAGCCAGGAATCTACATGGGT | |
| AGATGAGATAGACACAGACCTGCTCCCCGCAGCCTTGTTGAGAGCCACACTTCTGCCCAT | |
| GCCAGGAGCCAGCTGTGTGACCATCCAGGGGTGGAGGGGGAAAACCAGGCAATTTCGTTC | |
| CTGGAATCAACCAAATCATGTTTTCCTCTTGGATGGAAGTGTCAAAGGCAGAAGGGTGTG | |
| GGAGGGGGACAAGGTCAGTATTTACCAAAGTGTATCTGATTTTAAAAATTCCTTTAGTCT | |
| GTAAAACTCCTAGAGGGAGGGAGGTAACTGAATTCACTTCTTTTTGTGGATCGTATCAAG | |
| GTCACTGGGTTTTACTGGCTGGTGCTGGGAAAATGAAGCTAAGTGAGGAGCTTCCATTGG | |
| AATGCTTTTCCAGGGAGAGAGGCCAGTTAATTTAAAAAAAACAGTCGCTAGTTAACAGCG | |
| ACAGAGCCCAGCACCCTGGGGTCTTTGTGAATATCCAGACTGTTTCAGCCCAGCCCATCT | |
| CAGCCAACCCTCCTTAGACTGAGCTGTCAGAGCAAGCAATTAGGGGCCAGCCTGCCTCCA | |
| CCTCCCACCCCCTTCCACCTCCATCAGTCATGTGTGCAGAGTCAGTGCTCGGGATCCCGG | |
| GCCCAGCTTTTGCCTTTTTGGGGATGCTTGGTGAGACAGATTTGCCAGTCAGCCCTTTTG | |
| AGTTCCCGCCTCACCCAGGGGCTCCCAGCCTGCACTTGCAGGAGTGGTGATGCCCCAAGT | |
| CTGCGAATCCAGGGTGCACGTGGTCAATATCCCCTCCTGCATTCAGGAGAGCCATGGTAG | |
| GGCTGGAGTTGGGTCTTGCCCAGCCCTGCAGTTTCATAGTCCCAGCCTTCCTGGTGCTGG | |
| GGAGGGAGGACTGTGAATGGCTGTTCTCCCCTCACTGCTGAGTCTCCCAGGACCCCCTTT | |
| GGAGATGCCCATGGCATGGGCACTGCCCACAGGCTCAGCCAGAACCTCTTGGTGTACCCG | |
| ATAAGCTGCAGGTTATCCCTTGCTCTGTGCGCCTTTTATTTGTCCTTAAACTACCTCCTT | |
| AGAGCTCTGAAGGGGTCTCCTAGTTCCAGATTTTAATTTGGGGAACAGATCTGGGTTCTT | |
| TTTAACCCTCTTCTTTCTCAGTCTATGAGAAACTTGCCCTGAGGGGCACCTGGGCTAGGG | |
| GCTTGGGACTGGAAGACCATCCCCGCCTTGTGCCACAACTTTGGTCATGGGATCTGCTCT | |
| TTGTCATTCTTAGCCCCCTACTGTGGCCCCCATAGCCCCATAACCCAGAGAGGGAGCTGG | |
| ACTTCAGGGAGCCTGAGTGATGCTTTCCCAGGAGCAGGGCAGCTGGCTGGACCAGAAAGT | |
| AGAGGGCCCATGGGAGTGACTGCACCCTTGGTGGCTGCTGGAAGGGGAGAGGTTCTCAGC | |
| ATCAGGCCACCTCCACCCCAATGCCAGGATAGATGTATTCTAGAGTAGGGGTGGAGGCGG | |
| CCCAGGAGGCTGAAGACAGGTGCACAGATGCTTCCCACGACCTTGCCATTTGGGGTGGGC | |
| TCTTCAACATCTCAGGCTGTGGCTGGAACAGGACAGGATGATCTAAAACACACGTACCAT | |
| TGGCTGTAAAACAGTATGAGCCCAGACTGACGCTGAAATCCCTCATGAGCCAACCTTAGC | |
| TACAAGGTAGGGAGTTCTGAGGGAAGCCGCGTGCTCCTCAGGAGAGAGCTGTTTAGGTTT | |
| TCCGATCTTTTTGCTCAGGGGCCAAACACTGAAGGCACGTACTGCCCAACCCACTGAGCG | |
| CCTGAGGCCATTCCCTCCTTTTCCGCATGCCTCCTGCCTCCTGGGCTATTCCTCTCCACC | |
| CAGAAGGCTGGGAATCCCAGCTGATTCCCTGACAGGAGCCGACTTCACACACAGGTGACT | |
| CTCAGGCATTGGCTCATGTTTTCAGCCAGGGATAAACCATCCCTTCTTGGGGCTTTAAGT | |
| CCCTGGGGAGCTTTCCCTGTAGGTCTCCTGGGTGTTGAGAGACAAGTTGGAGACCAACCT | |
| CCAATGAATGAGCCGCGGTCATTCATTAATTCACTCACGTAATTTACTGAGTAGCTGCAA | |
| CATGCCAGCCTCTACGTTAGGTTCTGCGGATAAAGGAGGAATAAGACAGAGTCAGGAGAA | |
| CTGTTCCTTGTGGTTTCCGTCCCTTGGGGACCACAGGCATCAGCAGTCCCATTCAAGTCA | |
| CCTGAGGCAAAGTGTCTGCATCTTCGTCCAGCGACCCTTTGCTTTTCGGCTCCTAGAATC | |
| CTTAGAGTCTGAATTCCTTTAGCTGGGAACAGCTGTCATGGTCACCCCTGGATAACATTT | |
| GCCACCAAGTATAGATGCTGGATCTTGGGTTCCAGGCAGACATCATCCAGGTCCATCTGG | |
| AACTTTCAGTGATAGCTGCCTTCAGCCAGCATCTTTGGGGGACTCTATAATAGCAGCTTG | |
| AGATCAGTGTCTAGAAGACTGTTCTGCAATTTGCTGCCAAATGCATCTCAGGTTTTTAAA | |
| GTCATTGTTTCTTGCTCATGGTGGCTCATTTATTACATAGTCCCCTCACCCCACTAATGG | |
| ATAATGGGAGGAAAAGTTGCTGCTTCCTTCAGCATCAAAGCCTTTCCTTGGGAATCTGCC | |
| TCCCTCCATGGCAGGGGTGGATTCGGGAGCTGGGAGTAACCAGGCAAAGTCAACCAGATG | |
| CCTAGCTCCTGCTGAGACCCAGGTCCTATGGCAGCTCCTCATTAGATTAAAGGAGACCAC | |
| TTCCAAAGCAGGTGCTGCATGGCTCACCATCATATGCCCCAAACAACTGAAAGTTGGCGG | |
| TTATCACCAGACTGTGAGTTTCTGGCAAGTAGCTTGGGGAAGCTGAATAAACTCTAGGCC | |
| CAGGGCTACTAAAGACTTCAGGATAGAATTCTCCATCAAATATACAGCATAAGTAAAACT | |
| GCTCTGCACTGTTTAATCCATTTCCAAGGGGCTTAGAAAAGCTAACAAGGGTGTGTCCCC | |
| TGTCCTGCCCCACCGGTTTGCTGGCTTTGTAATAACATAAGACCATTGTGGTTGTTGGTG | |
| TCAGATACCTTCCCATCCTGAGCTCTCTCACCTACCTGCTCTCTCTCCTAGAGCAGGATA | |
| CTGGGGTACTTTTAAGAAGGGTGCTCCTTTTAAGATGCCCAGAAAAGCTGTATTTAACTC | |
| TTGCTATTTGTAACTTGGGGATGGTCTCCCCTGCCCCAGGGCACATAAGAGCAAAGGCTC | |
| CAATGGTCAGTGGATGACTCTGCAAAAGTGACCCCCTGTGCCAGAAGCTATAGCCCTCTC | |
| CCCAACAGGTCTCTCTTGTTGGCCAGAGGGCCTGCTTCCCATGGGCATTGCAAGTGCCAC | |
| CGTGCGGGGCCTGGCTCTGCACACCCAGGAAAAGTCTGCAGACCCCCAGCCCTCCGCAAT | |
| AATTCACCAGACCAGAAGCCACTGGTGTACAGAGAACACTTAAAAAAATGTATTTTATGT | |
| GAAAAAAAATTAAAACTCTGTATACTGTATCAGCAGCTTTGTGTAAAAATGGCAATCAAG | |
| AGAGTCTAATATATTTAAAACTTTTTTAAAAAAAATCTTCGCAGATCTTTGATATCGTAC | |
| TGAGGTAACTTCCACGTAGCCCCTTGCCACGCGGCACCGGTGGGCCTTGGGTCCAAAACT | |
| GTGGCTCAGCCACATCCCAAAGGGGGCACATGTCCCTGGAGTTGCTTCCAGCTGCCAAGG | |
| CCTGTGACAGAATTCGCTGTTAAGAGTTTTTAATTAAAATTATTAAATTCCTTTTAATAA | |
| CAAAAAAAAAAAAAAAAAAAAAAAAAAAAAAAAAAAAAAAAAAAAAAA | |

In a search of public sequence databases, the NOV72 nucleic acid sequence, located on chromsome 20 has 5593 of 5597 bases (99%) identical to a gb:GENBANK-ID:AB020630|acc:AB020630.1 mRNA from Homo sapiens (Homo sapiens mRNA for KIAA0823 protein, partial cds). Public nucleotide databases include all GenBank databases and the GeneSeq patent database.

The disclosed NOV72 polypeptide (SEQ ID NO:172) encoded by SEQ ID NO:171 has 567 amino acid residues and is presented in Table 72B using the one-letter amino acid code. Signal P, Psort and/or Hydropathy results predict that NOV72 has no signal peptide and is likely to be localized in the nucleus with a certainty of 0.9800.

TABLE 72B
Encoded NOV72 protein sequence.

| | |
|---|---|
| MASHVDLLTELQLLEKVPTLERLRAAQKRRAQQLKKWAQYEQDLQHRKRKHERKRSTGGR | (SEQ ID NO:172) |
| RKKVSFEASVALLEASLRNDAEEVRYFLKNKVSPDLCNEDGLTALHQCCIDNFEEIVKLL | |
| LSHGANVNAKDNELWTPLHAAATCGHINLVKILVQYGADLLAVNSDGNMPYDLCEDEPTL | |
| DVIETCMAYQGITQIDINEMRVAPEQQMIADIHCMIAAGQDLDWIDAQGATLLHIAGANG | |
| YLRAAILLLDHGVRVDVKDWDGWEPLHAAAFWGQMQMAELLVSHGASLSARTSMDEMPID | |
| LCEEEEFKVLLLELKHKHDVIMKSQLRHKSSLSRRTSSAGSRGKVVRRASLSDRTNLYRK | |
| EYEGEAILWQRSAAEDQRTSTYNGDIRETRTDQENKDPNPRLEKPVLLSEFPTKIPRGEL | |
| DMPVENGLRAPVSAYQYALANGDVWKVHEVPDYSMAYGNPGVADATPPWSSYKEQSPQTL | |
| LELKRQRAAAKLLSHPFLSTHLGSSMARTGESSSEGKAPLIGGRTSPYSSNGTSVYYTVT | |
| SGDPPLLKFKAPIEEMEEKVHGCCRIS | |

A search of sequence databases reveals that the NOV72 amino acid sequence has have 412 of 412 amino acid residues (100%) identical to, and 412 of 412 amino acid residues (100%) similar to, the 412 amino acid residue ptnr:SPTREMBL-ACC:O94912 protein from Homo sapiens (Human) (KIAA0823 PROTEIN). Public amino acid databases include the GenBank databases, SwissProt, PDB and PIR.

NOV72 is expressed in at least Adrenal Gland/Suprarenal gland, Amygdala, Artery, Bone, Brain, Dermis, Heart, Hippocampus, Kidney, Lung, Lymph node, Lymphoid tissue, Mammary gland/Breast, Pancreas, Peripheral Blood, Small Intestine, Spleen, Stomach, Substantia Nigra, Synovium/Synovial membrane, Thalamus, Tonsils, Umbilical Vein, Urinary Bladder, and Uterus. This information was derived by determining the tissue sources of the sequences that were included in the invention including but not limited to SeqCalling sources, Public EST sources, Literature sources, and/or RACE sources.

The disclosed NOV72 polypeptide has homology to the amino acid sequences shown in the BLASTP data listed in Table 72C.

TABLE 72C
BLAST results for NOV72

| Gene Index/ | | Length | Identity | Positives | |
|---|---|---|---|---|---|
| Identifier | Protein/Organism | (aa) | (%) | (%) | Expect |
| gi\|14770818\|ref\|XP_ | KIAA0823 protein | 567 | 567/567 | 567/567 | 0.0 |
| 028840.1\| | [Homo sapiens] | | (100%) | (100%) | |
| (XM_028840) | | | | | |
| gi\|14029700\|gb\|AAK52795.1\| | CAAX box protein | 568 | 550/568 | 557/568 | 0.0 |
| AF362909_1\| | TIMAP [Bos | | (96%) | (97%) | |
| (AF362909 | taurus] | | | | |
| gi\|18157719\|gb\|AAL62093.1\| | protein | 568 | 547/568 | 556/568 | 0.0 |
| AF423761_1 | phosphatase 1 | | (96%) | (97%), | |
| (AF423761) | regulatory | | | | |
| | subunit 16B [Mus | | | | |
| | musculus] | | | | |
| gi\|4240132\|dbj\|BAA74846.1\| | KIAA0823 protein | 412 | 412/412 | 412/412 | 0.0 |
| (AB020630 | [Homo sapiens] | | (100%) | (100%) | |
| gi\|9368796\|emb\|CAB98284.1\| | dJ1076E17.1 | 411 | 411/411 | 411/411 | 0.0 |
| (AL121889) | (KIAA0823 protein | | (100%) | (100%) | |
| | (continues in | | | | |
| | AL023803)) [Homo | | | | |
| | sapiens] | | | | |

Table 72D lists the domain descriptions from DOMAIN analysis results against NOV72. This indicates that the NOV72 sequence has properties similar to those of other proteins known to contain this domain.

TABLE 72D
Domain Analysis of NOV72

gnl|Pfam|pfam00023, ank, Ank repeat. Ankyrin repeats
generally consist of a beta, alpha, alpha, beta order of
secondary structures. The repeats associate to form a higher
order structure.
CD-Length = 33 residues, 100.0% aligned
Score = 47.4 bits (111), Expect = 2e−06

Ankyrin repeats are tandemly repeated modules of about 33 amino acids. They occur in a large number of functionally diverse proteins mainly from eukaryotes. The few known examples from prokaryotes and viruses may be the result of horizontal gene transfers. The conserved fold of the ankyrin repeat unit is known from several crystal and solution structures, e.g. from: p53-binding protein 53BP2, cyclin-dependent kinase inhibitor p19Ink4d, transcriptional regulator GABP-beta, and NF-kappaB inhibitory protein IkB-alpha. It has has been described as an L-shaped structure consisting of a beta-hairpin and two alpha-helices. Many ankyrin repeat regions are known to function as protein-protein interaction domains.

The disclosed NOV72 nucleic acid of the invention encoding a ankyrin repeat containing protein-like protein includes the nucleic acid whose sequence is provided in Table 72A or a fragment thereof. The invention also includes a mutant or variant nucleic acid any of whose bases may be changed from the corresponding base shown in Table 72A while still encoding a protein that maintains its ankyrin repeat containing protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV72 protein of the invention includes the ankyrin repeat containing protein-like protein whose sequence is provided in Table 72B. The invention also includes a mutant or variant protein any of whose residues may be changed from the corresponding residue shown in Table 72B while still encoding a protein that maintains its ankyrin repeat containing protein-like activities and physiological functions, or a functional fragment thereof. In the mutant or variant protein, up to about 0 percent of the residues may be so changed.

The invention further encompasses antibodies and antibody fragments, such as F_{ab} or (F_{ab})₂, that bind immunospecifically to any of the proteins of the invention.

The above defined information for this invention suggests that this ankyrin repeat containing protein-like protein (NOV72) may function as a member of a “ankyrin repeat containing protein family”. Therefore, the NOV72 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV72 nucleic acids and proteins of the invention are useful in potential therapeutic applications implicated in diseases including but not limited to various pathologies and disorders as indicated below. For example, a cDNA encoding the ankyrin repeat containing protein-like protein (NOV72) may be useful in gene therapy, and the ankyrin repeat containing protein-like protein (NOV72) may be useful when administered to a subject in need thereof. By way of nonlimiting example, the compositions of the present invention will have efficacy for treatment of patients suffering from Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Systemic lupus erythematosus, Autoimmune disease, Asthma, Emphysema, Scleroderma, allergy, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Multiple sclerosis, Ataxia-telangiectasia, Leukodystrophies, Behavioral disorders, Addiction, Anxiety, Pain, Neuroprotection, or other pathologies or conditions. The NOV72 nucleic acid encoding the ankyrin repeat containing protein-like protein of the invention, or fragments thereof, may further be useful in diagnostic applications, wherein the presence or amount of the nucleic acid or the protein are to be assessed.

NOV72 nucleic acids and polypeptides are further useful in the generation of antibodies that bind immuno-specifically to the novel NOV72 substances for use in therapeutic or diagnostic methods. These antibodies may be generated according to methods known in the art, using prediction from hydrophobicity charts, as described in the “Anti-NOVX Antibodies” section below. The disclosed NOV72 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV73

A disclosed NOV73 nucleic acid of 1001 nucleotides (also referred to as CG57313-01) encoding a GPCR-like protein is shown in Table 73A. The start and stop codons are in bold letters.

TABLE 73A
NOV73 nucleotide sequence.

(SEQ ID NO:173)

CATGGATTTCCCTTAAGAAAAAACAGAATGATCAATGATAGCCACTTCAGTGGTTTTATACTCCTTGGAT
TCACAGGGCAGCCTCAGCTTCAGATGATGATCTCTGGGGTTGTCTTTTTCTTCTACACTATTGCCTTCAT
GGGAAATATGGCCATCATCCTATTGTCTTTCCTAGATGACCATCTCCAAGTCCCCATGTACTTCTTCCTT
AGAAATTTGGCCATCTTGGATCTCTGTTATACCACAAATATAGTCCCACAAATGTTGGTCAGTATCTGGG
GCAAAGACAAAAGAATTACCTTTGGTGGGTGTGCCTTTCAACTTTTCATTGATGTGGCACTGTACTCAGT
TGAATGCATCCTTCTGTCCATGATGTCATATGATCGACTCAATGCTATCTGCAAGCCTCTGCATCATATG
ACCATAATGAACCTCCAACTCTGCCAGGGCCTTGTGGTCATCTCCTGGGTAGTTGGTGTGATTAATTGCA
TCATACCTTCCCCTTATGCCACGAGTCTTCCTCGATGTAGGAACCACCACCTAGACCACTTTTTTGTGTG
TGTGAAATGTCTGCAAAGATCAAGATTCAAGATTGCATGTGTGGACACCACAGCCATGGAGGTAACCACA
TTTGCCATGTGCCTGATTATAGTTCTTGTTCCTCTTCTTCTTATTCTTGTGTCATATGGTTTCATTGCTG
TGGCTGTACTCAAGATCAAGTCTGCAGCAGGAAGACAAAAAGCATTTGGGACCTGTTCCTCCCATCTCGT
TGTGGTATCCATCTTCTGTGGGACAGTTACATACATGTATATACAGCCAGGAAACAGTCCAAATCAGAAT
GAGGGCAAACTTCTCAGTATATTTTACTCCATTGTTACTCCCAGCTTGAACCCATTAATTTATACGGTAA
GGAATAAGGAGTTCAAGGGGGCCATGAAGAGGCTAACTGGAAAAGAAAAAGATTGCATGGAAAAAAGAGG
ACATTGATTCTTCCTCCCAGCAATTTCTAAT

In a search of public sequence databases, the NOV73 nucleic acid sequence, located on chromsome 6 has has 223 of 363 bases (61%) identical to a gb:GENBANK-ID:U86270|acc:U86270.1 mRNA from Homo sapiens (Homo sapiens olfactory receptor (OR5-40) gene, partial cds). Public nucleotide databases include all GenBank databases and the GeneSeq patent database.

The disclosed NOV73 polypeptide (SEQ ID NO:174) encoded by SEQ ID NO:173 has 319 amino acid residues and is presented in Table 73B using the one-letter amino acid code. Signal P, Psort and/or Hydropathy results predict that NOV73 has a signal peptide and is likely to be localized extracellularly with a certainty of 0.6400. The most likely cleavage site for a NOV73 peptide is between amino acids 42 and 43.

TABLE 73B
Encoded NOV73 protein sequence.

| | |
|---|---|
| MINDSHFSGFILLGFTGQPQLQMMISGVVFFFYTIAFMGNMAIILLSFLDDHLQVPMYFF | (SEQ ID NO:174) |
| LRNLAILDLCYTTNIVPQMLVSIWGKDKRITFGGCAFQLFIDVALYSVECILLSMMSYDR | |
| LNAICKPLHHMTIMNLQLCQGLVVISWVVGVINCIIPSPYATSLPRCRNHHLDHFFVCVK | |
| CLQRSRFKIACVDTTAMEVTTFAMCLIIVLVPLLLILVSYGFIAVAVLKIKSAAGRQKAF | |
| GTCSSHLVVVSIFCGTVTYMYIQPGNSPNQNEGKLLSIFYSIVTPSLNPLIYTVRNKEFK | |
| GAMKRLTGKEKDCMEKRGH | |

A search of sequence databases reveals that the NOV73 amino acid sequence has have 165 of 304 amino acid residues (54%) identical to, and 226 of 304 amino acid residues (74%) similar to, the 320 amino acid residue ptnr:SPTREMBL-ACC:Q9Y3N9 protein from Homo sapiens (Human) (DJ88J8.1 (NOVEL 7 TRANSMEMBRANE RECEPTOR (RHODOPSIN FAMILY) (OLFACTORY RECEPTOR LIKE) PROTEIN) (HS6M1-15))). Public amino acid databases include the GenBank databases, SwissProt, PDB and PIR.

The disclosed NOV73 polypeptide has homology to the amino acid sequences shown in the BLASTP data listed in Table 73C.

TABLE 73C
BLAST results for NOV73

| Gene Index/ | | Length | Identity | Positives | |
|---|---|---|---|---|---|
| Identifier | Protein/Organism | (aa) | (%) | (%) | Expect |
| gi\|18480000\|gb\|AAL61014.1\| | olfactory | 308 | 169/307 | 232/307 | 8e−88 |
| (AY073351) | receptor MOR256-8 | | (55%) | (75%) | |
| | [Mus musculus] | | | | |
| gi\|13624329\|ref\|NP_ | olfactory | 320 | 165/304 | 226/304 | 5e−86 |
| 112165.1\| | receptor, family | | (54%) | (74%) | |
| (NM_030903) | 2, subfamily W, | | | | |
| | member 1 [Homo | | | | |
| | sapiens] | | | | |
| gi\|18480974\|gb\|AAL61501.1\| | olfactory | 312 | 168/304 | 227/304 | 5e−86 |
| (AY073838 | receptor MOR256- | | (55%) | (74%) | |
| | 31 [Mus musculus] | | | | |
| gi\|12054429\|emb\|CAC20522.1\| | olfactory | 320 | 165/304 | 225/304 | e−85 |
| (AJ302602) | receptor [Mus | | (54%) | (73%) | |
| | musculus] | | | | |
| gi\|12054431\|emb\|CAC20523.1\| | olfactory | 320 | 164/304 | 226/304 | e−85 |
| (AJ302603) | receptor [Homo | | (53%) | (73%) | |
| | sapiens] | | | | |

Table 73D lists the domain descriptions from DOMAIN analysis results against NOV73. This indicates that the NOV73 sequence has properties similar to those of other proteins known to contain this domain.

TABLE 73D
Domain Analysis of NOV73

gnl|Pfam|pfam00001, 7tm_1, 7 transmembrane receptor
(rhodopsin family).
CD-Length = 254 residues, 100.0% aligned
Score = 92.0 bits (227), Expect = 4e−20

The protein sequence fingerprint is potently diagnostic of all sequences of this type in the database in which it was derived (the OWL composite sequence database, version 8.1), and has continued to perform well on subsequent database updates, identifying 240 receptors in OWL17.0. Results are compared with a commonly used pattern template for this class of receptors. The investigation suggests that discriminating power is improved in the fingerprint approach because the recognition of individual features is made mutually conditional. Furthermore, by avoiding the definition of predetermined feature separations, members of protein families possessing all or only part of the fingerprint may be identified. PMID: 8386361

The fingerprint encodes the seven putative membrane-spanning motifs and was potently diagnostic of all GPCRs (52 in all) in version 8.1 of the OWL composite sequence database, readily distinguishing them from all other integral membrane proteins. With a 3-fold increase in the size of OWL, the fingerprint has been updated and now finds 332 receptors that match all the motifs.

The glucagon receptor is a member of a distinct class of G protein-coupled receptors (GPCRs) sharing little amino acid sequence homology with the larger rhodopsin-like GPCR family. To identify the components of the glucagon receptor necessary for G-protein coupling, sequentially all or part of each intracellular loop (i1, i2, and i3) and the C-terminal tail of the glucagon receptor were replaced with the 11 amino acids comprising the first intracellular loop of the D4 dopamine receptor.

Whereas numerous mutations of the human lutropin receptor (hLHR) and human TSH receptor (hTSHR) have been shown to cause constitutive activation of these receptors, it has been suggested that either the hFSHR as a whole, or the i3/TM VI region of the hFSHR, is less susceptible to mutation-induced constitutive activation.

The disclosed NOV73 nucleic acid of the invention encoding a GPCR-like protein includes the nucleic acid whose sequence is provided in Table 73A or a fragment thereof. The invention also includes a mutant or variant nucleic acid any of whose bases may be changed from the corresponding base shown in Table 73A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 39 percent of the bases may be so changed.

The disclosed NOV73 protein of the invention includes the GPCR-like protein whose sequence is provided in Table 73B. The invention also includes a mutant or variant protein any of whose residues may be changed from the corresponding residue shown in Table 73B while still encoding a protein that maintains its GPCR-like activities and physiological functions, or a functional fragment thereof. In the mutant or variant protein, Up to about 46 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV73) may function as a member of a “GPCR family”. Therefore, the NOV73 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV73 nucleic acids and proteins of the invention are useful in potential therapeutic applications implicated in diseases including but not limited to various pathologies and disorders as indicated below. For example, a cDNA encoding the GPCR-like protein (NOV73) may be useful in gene therapy, and the GPCR-like protein (NOV73) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV73 nucleic acid encoding the GPCR-like protein of the invention, or fragments thereof, may further be useful in diagnostic applications, wherein the presence or amount of the nucleic acid or the protein are to be assessed.

NOV73 nucleic acids and polypeptides are further useful in the generation of antibodies that bind immuno-specifically to the novel NOV73 substances for use in therapeutic or diagnostic methods. These antibodies may be generated according to methods known in the art, using prediction from hydrophobicity charts, as described in the “Anti-NOVX Antibodies” section below. The disclosed NOV73 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV74

A disclosed NOV74 nucleic acid of 1008 nucleotides (also referred to as CG57315-01) encoding a GPCR-like protein is shown in Table 74A. The start and stop codons are in bold letters.

TABLE 74A
NOV74 nucleotide sequence.

(SEQ ID NO:175)

TTCACAGCTGATATTCAGAAAATAGCAAAAATGATCAATGATAGTTACTTTGGTTGGCTTATGCTCCTTG
GGTTCCCTGGGAAGCCTCAGCTGGAGATGATCATCTCTGGGGTTGTCTTTTTCTTCTATGCAATTTCTTT
GATGGGAAATATGGTCCTTATCCTGCTGCCATTACTGGATAAACATCTCCAAACCCCCATATATTTCTTT
CTTAGAAATCTGGCTATCTTGGATCTTTGTTACACCACAAATATAGTCCCACAGATGTTGGTCAATGCCT
GGGGTAAAGACAAGAAAATCACTTTTGGTGGCTGTGCTTTTCAACTTTTCACTAATGTGACGCTATGCAC
GGTTGAATGTATGCTTCTGGCTGTGATGTCATATGACCCATTCAATGCTGTCTGCAAGCCTCTGGACTAT
ATGACCATAATGAACCCCCAACTCTGTCAAGGCCTGGTGGCCATGACCTGGTTAATTGGTGTCACTAATT
GCATGATACTTTCCCCCTGTCCTGTGAGTCTTCCTCGATGCGGAGACCACCACCTGGATCACTATTTTTG
TGAAATATCTGCAATGGTCAAAATTGCATGTGGGGCTACCACAGTCATGGAGTTGCATTGTGTTGTTGTT
GTTGTTTTCATTTTCCTTGCATCACTTCTTCTCATTCTTGTGTCATATGGCTTCATTGCTGTGGCTGTAC
TCAAGATCAAGTCTGCAGCAGGAAGACAAAAAGCATTTGGGACCTGTTTCTCCCATCTCATTGTGGTATC
CATCTTCTATGGGACTGTTAGATATATGTATATAGAGCCAGGAAACAGTCCATCTCAGGATGAGGGCAAA
CTTCTCCATATATTTTACTCCATTGTTACTCCCACCTTGAACCCAATCCCACTAAGGAATAAGGAGTTCA
AGTGGGCCATGAAAAGGCTTATTGGAAAAGAAAAAGGTTCTGGAGACACAATAGGTCACTAACATCTTTT
TACAAGAAATTCCTGGCCGGGCACGGTG

In a search of public sequence databases, the NOV74 nucleic acid sequence, located on chromsome 6 has has 583 of 946 bases (61%) identical to a gb:GENBANK-ID:RATOL1RECE|acc:L34074.1 mRNA from Rattus norvegicus (Rat OL1 receptor gene, complete cds). Public nucleotide databases include all GenBank databases and the GeneSeq patent database.

The disclosed NOV74 polypeptide (SEQ ID NO:176) encoded by SEQ ID NO:175 has 313 amino acid residues and is presented in Table 74B using the one-letter amino acid code. Signal P, Psort and/or Hydropathy results predict that NOV74 has a signal peptide and is likely to be localized extracellularly with a certainty of 0.6000. The most likely cleavage site for a NOV74 peptide is between amino acids 63 and 64.

TABLE 74B
Encoded NOV74 protein sequence.

| | |
|---|---|
| MINDSYFGWLMLLGFPGKPQLEMIISGVVFFFYAISLMGNMVLILLPLLDKHLQTPIYFF | (SEQ ID NO:176) |
| LRNLAILDLCYTTNIVPQMLVNAWGKDKKITFGGCAFQLFTNVTLCTVECMLLAVMSYDP | |
| FNAVCKPLDYMTIMNPQLCQGLVAMTWLIGVTNCMILSPCPVSLPRCGDHHLDHYFCEIS | |
| AMVKIACGATTVMELHCVVVVVFIFLASLLLILVSYGFIAVAVLKIKSAAGRQKAFGTCF | |
| SHLIVVSIFYGTVRYMYIEPGNSPSQDEGKLLHIFYSIVTPTLNPIPLRNKEFKWAMKRL | |
| IGKEKGSGDTIGH | |

A search of sequence databases reveals that the NOV74 amino acid sequence has 161 of 298 amino acid residues (54%) identical to, and 220 of 298 amino acid residues (73%) similar to, the 320 amino acid residue ptnr:SPTREMBL-ACC:Q9Y3N9 protein from Homo sapiens (Human) (DJ88J8.1 (NOVEL 7 TRANSMEMBRANE RECEPTOR(RHODOPSIN FAMILY) (OLFACTORY RECEPTOR LIKE) PROTEIN) (HS6M1-15))). Public amino acid databases include the GenBank databases, SwissProt, PDB and PIR.

NOV74 is expressed in at least Whole Organism. Expression information was derived from the tissue sources of the sequences that were included in the derivation of the sequence of CG57315_{—}01. The sequence is predicted to be expressed in the following tissues because of the expression pattern of (GENBANK-ID: gb:GENBANK-ID:RATOL1RECE|acc: L34074.1) a closely related Rat OL1 receptor gene, complete cds homolog in species Rattus norvegicus: heart. This information was derived by determining the tissue sources of the sequences that were included in the invention including but not limited to SeqCalling sources, Public EST sources, Literature sources, and/or RACE sources.

The disclosed NOV74 polypeptide has homology to the amino acid sequences shown in the BLASTP data listed in Table 74C.

TABLE 74C
BLAST results for NOV74

| Gene Index/ | | Length | Identity | Positives | |
|---|---|---|---|---|---|
| Identifier | Protein/Organism | (aa) | (%) | (%) | Expect |
| gi\|17464347\|ref\|XP_ | similar to | 590 | 186/186 | 186/186 | e−100 |
| 069460.1\| | dM538M10.7 (novel | | (100%) | (100%) | |
| (XM_069460) | 7 transmembrane | | | | |
| | receptor | | | | |
| | (rhodopsin | | | | |
| | family) | | | | |
| | (olfactory | | | | |
| | receptor like) | | | | |
| | protein) [Homo | | | | |
| | sapiens] | | | | |
| gi\|118480974\|gb\|AAL61501.1\| | olfactory | 312 | 167/293 | 218/293 | e−84 |
| (AY073838) | receptor MOR256- | | (56%) | (73%) | |
| | 31 [Mus musculus] | | | | |
| gi\|18480000\|gb\|AAL61014.1\| | olfactory | 308 | 159/303 | 225/303 | 2e−83 |
| (AY073351 | receptor MOR256-8 | | (52%) | (73%) | |
| | [Mus musculus] | | | | |
| gi\|13624329\|ref\|NP_ | olfactory | 320 | 161/301 | 219/301 | 3e−83 |
| 112165.1\| | receptor, family | | (53%) | (72%) | |
| (NM_030903) | 2, subfamily W, | | | | |
| | member 1 [Homo | | | | |
| | sapiens] | | | | |
| gi\|12054429\|emb\|CAC20522.1\| | olfactory | 320 | 161/301 | 218/301 | 6e−83 |
| (AJ302602) | receptor [Homo | | (53%) | (71%) | |
| | sapiens] | | | | |

Table 74D lists the domain descriptions from DOMAIN analysis results against NOV74. This indicates that the NOV74 sequence has properties similar to those of other proteins known to contain this domain.

TABLE 74D
Domain Analysis of NOV74

gnl|Pfam|pfam00001, 7tm_1, 7 transmembrane receptor
(rhodopsin family).
CD-Length = 254 residues, 99.2% aligned
Score = 96.3 bits (238), Expect = 2e−21

The disclosed NOV74 nucleic acid of the invention encoding a GPCR-like protein includes the nucleic acid whose sequence is provided in Table 74A or a fragment thereof. The invention also includes a mutant or variant nucleic acid any of whose bases may be changed from the corresponding base shown in Table 74A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 39 percent of the bases may be so changed.

The disclosed NOV74 protein of the invention includes the GPCR-like protein whose sequence is provided in Table 74B. The invention also includes a mutant or variant protein any of whose residues may be changed from the corresponding residue shown in Table 74B while still encoding a protein that maintains its GPCR-like activities and physiological functions, or a functional fragment thereof. In the mutant or variant protein, up to about 46 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV74) may function as a member of a “GPCR family”. Therefore, the NOV74 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV74 nucleic acids and proteins of the invention are useful in potential therapeutic applications implicated in diseases including but not limited to various pathologies and disorders as indicated below. For example, a cDNA encoding the GPCR-like protein (NOV74) may be useful in gene therapy, and the GPCR-like protein (NOV74) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV74 nucleic acid encoding the GPCR-like protein of the invention, or fragments thereof, may further be useful in diagnostic applications, wherein the presence or amount of the nucleic acid or the protein are to be assessed.

NOV74 nucleic acids and polypeptides are further useful in the generation of antibodies that bind immuno-specifically to the novel NOV74 substances for use in therapeutic or diagnostic methods. These antibodies may be generated according to methods known in the art, using prediction from hydrophobicity charts, as described in the “Anti-NOVX Antibodies” section below. The disclosed NOV74 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV75

A disclosed NOV75 nucleic acid of 1050 nucleotides (also referred to as CG57317-01) encoding a GPCR-like protein is shown in Table 75A. The start and stop codons are in bold letters.

TABLE 75A
NOV75 nucleotide sequence.

(SEQ ID NO:177)

ACTTTTTAAAGATTGATATTTTGCCCAATGGCCAACACATTATCCTCCCTGAATTCTTGTAATGTGTTTC
TCCTAGTTCTGAACAGGGTGATGGGCATGACCAACAGCAGTGTCAAGGGAGACTTCATCCTGGTGGGTTT
CTCTAGTTCTGAACAGGGTGATGGGCATGACCAACAGCAGTGTCAAGGGAGACTTCATCCTGGTGGGTTT
GGAAATACAGTAATTATTCTGATCTGCTCTGTAGACCCTAAACTCAAGACACCCATGTATTTTTTCTTAA
CTCACCTCTCCTTAGTTGATATCTGTTTTACCACCAGTATTGTCCCCCAGCTGCTGTGGAACCTAAAAGG
ACCTGACAAAACAATCACATTCCTGGGTTGTGTCATCCAGCTCTACATCTCCCTGGCATTGGGCTCCACT
GAGTGTGTCCTCCTGGCTGTAATGGCTTTTGATCGCTATGCTGCAGTTTGCAAACCTCTCCACTATACCG
CCGTAATGAACCCTCAGCTGTGCCAGGCTCTGGCAGGGGTTGCGTGGCTGAGTGGAGTGGGAAACACTCT
TATCCAGGGCACTGTCACCCTCTGGCTTCCTCGCTGTGGACACCGATTGCTCCAACATTTTCTTCGTGAG
GTACCAGGGCACTGTCACCCTCTGGCTTCCTCGCTGTGGACACCGATTGCTCCAACATTTTCTTCGTGAG
TGGTCTTGCTCCTCTTGCCCTTAGTGCTAATACTGCTGTCCTATGGACATATAGCCAAGGTGGTCATAAG
GATCAAGTCAGTCCAGGCCTGGTGCAAAGGCCTGGGGACATGTGGATCCCATTTGATAGTAGTGTCCCTC
TTCTGTGGGACCATCACAGCTGTCTACATCCAGTCCAACAGTTCTTATGCCCATGCTCATGGGAAGTTCA
GAAAGGAGCACTGCGATTATTTAACAGAGACTTAGGCACATAAAAAATGAAGCAGAGTACACAGCGCTCA

In a search of public sequence databases, the NOV75 nucleic acid sequence, located on chromsome 6 has 611 of 912 bases (66%) identical to a gb:GENBANK-ID:HUMORLMHC|acc:L35475.1 mRNA from Homo sapiens (Human olfactory receptor-like gene, complete cds). Public nucleotide databases include all GenBank databases and the GeneSeq patent database.

The disclosed NOV75 polypeptide (SEQ ID NO:178) encoded by SEQ ID NO:177 has 331 amino acid residues and is presented in Table 75B using the one-letter amino acid code. Signal P, Psort and/or Hydropathy results predict that NOV75 has a signal peptide and is likely to be localized extracellularly with a certainty of 0.6400. The most likely cleavage site for a NOV75 peptide is between amino acids 23 and 24.

TABLE 75W
Encodcd NOV75 protein sequence.

| | |
|---|---|
| MANTLSSLNSCNVFLLVLNRVMGMTNSSVKGDFILVGFSHQPHLEKILFVAVLISYLLTL | (SEQ ID NO:178) |
| VGNTVIILICSVDPKLKTPMYFFLTHLSLVDICFTTSIVPQLLWNLKGPDKTITFLGCVI | |
| QLYISLALGSTECVLLAVMAFDRYAAVCKPLHYTAVMNPQLCQALAGVAWLSGVGNTLIQ | |
| GTVTLWLPRCGHRLLQHFLREVPSMIKLACVDIHDNEVQLFVASLVLLLLPLVLILLSYG | |
| HIAKVVIRIKSVQAWCKGLGTCGSHLIVVSLFCGTITAVYIQSNSSYAHAHGKFISLFYT | |
| VVTPTLNPLIYTLRNNDVKGALRLFNRDLGT | |

A search of sequence databases reveals that the NOV75 amino acid sequence has 178 of 306 amino acid residues (58%) identical to, and 234 of 306 amino acid residues (76%) similar to, the 312 amino acid residue ptnr:SPTREMBL-ACC:Q9R0Z2 protein from Mus musculus (Mouse) (573K1.3 (MM17M1-4 (NOVEL 7 TRANSMEMBRANE RECEPTOR (RHODOPSIN FAMILY) (OLFACTORY RECEPTOR LIKE) PROTEIN))). Public amino acid databases include the GenBank databases, SwissProt, PDB and PIR.

The disclosed NOV75 polypeptide has homology to the amino acid sequences shown in the BLASTP data listed in Table 75C.

TABLE 75C
BLAST results for NOV75

| Gene Index/ | | Length | Identity | Positives | |
|---|---|---|---|---|---|
| Identifier | Protein/Organism | (aa) | (%) | (%) | Expect |
| gi\|17464347\|ref\|XP_ | similar to | 590 | 270/322 | 271/322 | e−121 |
| 069460.1\| | dM538M10.7 (novel | | (83%) | (83%) | |
| (XM_069460) | 7 transmembrane | | | | |
| | receptor | | | | |
| | (rhodopsin | | | | |
| | family) | | | | |
| | (olfactory | | | | |
| | receptor like) | | | | |
| | protein) [Homo | | | | |
| | sapiens] | | | | |
| gi\|18480232\|gb\|AAL61130.1\| | olfactory | 309 | 254/308 | 273/308 | e−120 |
| (AY073467) | receptor MOR256-9 | | (82%) | (88%) | |
| | [Mus musculus] | | | | |
| gi\|18480518\|gb\|AAL61273.1\| | olfactory | 317 | 229/298 | 259/298 | e−112 |
| (AY073610) | receptor MOR256- | | (76%) | (86%) | |
| | 19 [Mus musculus] | | | | |
| gi\|18565094\|ref\|XP_ | hypothetical | 310 | 225/235 | 226/235 | e−112 |
| 094939.1\| | protein XP_094939 | | (95%) | (95%) | |
| (XM_094939) | [Homo sapiens] | | | | |
| gi\|14596252\|emb\|CAC43450.1\| | dM538M10.7 (novel | 317 | 233/307 | 260/307 | e−111 |
| (AL136158) | 7 transmembrane | | (75%) | (83%) | |
| | receptor | | | | |
| | (rhodopsin | | | | |
| | family) | | | | |
| | (olfactory | | | | |
| | receptor like) | | | | |
| | protein) [Mus | | | | |
| | musculus] | | | | |

Table 75D lists the domain descriptions from DOMAIN analysis results against NOV75. This indicates that the NOV75 sequence has properties similar to those of other proteins known to contain this domain.

TABLE 75D
Domain Analysis of NOV75

gnl|Pfam|pfam00001, 7tm_1, 7 transmembrane receptor
(rhodopsin family).
CD-Length = 254 residues, 46.9% aligned
Score = 78.6 bits (192), Expect = 5e−16

The disclosed NOV75 nucleic acid of the invention encoding a GPCR-like protein includes the nucleic acid whose sequence is provided in Table 75A or a fragment thereof. The invention also includes a mutant or variant nucleic acid any of whose bases may be changed from the corresponding base shown in Table 75A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 34 percent of the bases may be so changed.

The disclosed NOV75 protein of the invention includes the GPCR-like protein whose sequence is provided in Table 75B. The invention also includes a mutant or variant protein any of whose residues may be changed from the corresponding residue shown in Table 75B while still encoding a protein that maintains its GPCR-like activities and physiological functions, or a functional fragment thereof. In the mutant or variant protein, up to about 42 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV75) may function as a member of a “GPCR family”. Therefore, the NOV75 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV75 nucleic acids and proteins of the invention are useful in potential therapeutic applications implicated in diseases including but not limited to various pathologies and disorders as indicated below. For example, a cDNA encoding the GPCR-like protein (NOV75) may be useful in gene therapy, and the GPCR-like protein (NOV75) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV75 nucleic acid encoding the GPCR-like protein of the invention, or fragments thereof, may further be useful in diagnostic applications, wherein the presence or amount of the nucleic acid or the protein are to be assessed.

NOV75 nucleic acids and polypeptides are further useful in the generation of antibodies that bind immuno-specifically to the novel NOV75 substances for use in therapeutic or diagnostic methods. These antibodies may be generated according to methods known in the art, using prediction from hydrophobicity charts, as described in the “Anti-NOVX Antibodies” section below. The disclosed NOV75 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV76

A disclosed NOV76 nucleic acid of 1063 nucleotides (also referred to as CG57321-01) encoding a GPCR-like protein is shown in Table 76A. The start and stop codons are in bold letters.

TABLE 76A
NOV76 nucleotide sequence.

(SEQ ID NO:179)

TCTCTAATTCTCAGTGGCTTCCTCCTACTGTTGATGTCTATCCCTAACTGTGGGTATTTAGAGGTCTCAG
CTGGAATTTCACCTCCCAGTGCTAACATGTGGATCAACAATCAAAGCTCGCTAGATGATTTTATCCTATT
GGGATTTTCTGACCGTCCCTGGCTAGAGACACCCCTCTGTGTAATCTTTCTGGTGGCCTACATCTTTTCC
CTATTTGGAAATATCTCCATTATCCTAGTTTCCCATCTGGATCCCCAGCTTGACAGTCCCATGTACTTTT
TTGTCTCTAATCTATCCTTTCTGGACCTCTGCTATACCACCAGCACTGTCCCACAGATGCTGGTCAACCT
CCGGGGACCAGAAAAGACCATTAGCTATGGGGGTTGTGTTGCCCAACTCTATATATTTTTGGCCCTGGGT
TCTACTGAATGCATACTTCTAGCCATCATGGCCTTTGACCGTTACGCTGCCATATGCAAGCCCCTTCACT
ACCCAGTCATCATGAACCATAGACGCTGTATCCACATGGCTGCTGGCACTTGGATCAGTGGCTTTGCTAA
CTCCCTTGTCCAGTCCACTCTCACAGTGGTGGCCCCAAGATGTGGACAGAGGGTGTTGGACCATTTCTTC
TGTGAAGTTCCAGCCCTTTTGAAACTAGCCTGTATTGATATTCGTGTGAATGAAATGGAGCTCAATGTAC
TAGGCGCTTTGCTTCTCCTGATGCCACTCACCCTCATCCTGGGCACTTATGTGTTCATTGCTCAGGCAGT
AATGAGAATCTGCTCTGCTGAAAGTCGCTGGAAGGCTTTCAATACCTGTGCCTCACATTTGCTGGTGGTC
TCCCTCTTCTACTTCACAGCCATCAGTATGTATGTCCAGCCTCCCTCTAGCTATTCTCATGACCGGGGGA
AGATCATCATGGCTCTCTTTTATGGCATTGTCACACCCACCCTCAACCCATTCATCTACACATTGAGAAA
CAAGGATGTGAAAGCTGCCCTGAGAAGGTCACTGACTAAAGAGTTTTGGATTAAGACAAGATGATATCTG
AAAAGAAGTCCTA

In a search of public sequence databases, the NOV76 nucleic acid sequence, located on chromsome 6 has 657 of 971 bases (67%) identical to a gb:GENBANK-ID:RATOL1RECE|acc:L34074.1 mRNA from Rattus norvegicus (Rat OL1 receptor gene, complete cds). Public nucleotide databases include all GenBank databases and the GeneSeq patent database.

The disclosed NOV76 polypeptide (SEQ ID NO:180) encoded by SEQ ID NO:179 has 336 amino acid residues and is presented in Table 76B using the one-letter amino acid code. Signal P, Psort and/or Hydropathy results predict that NOV76 has a signal peptide and is likely to be localized localized extracellularly with a certainty of 0.6000. The most likely cleavage site for a NOV76 peptide is between amino acids 14 and 15.

TABLE 76B
Encoded NOV76 protein sequence.

| | |
|---|---|
| MSIPNCGYLEVSAGISPPSANMWINNQSSLDDFILLGFSDRPWLETPLCVIFLVAYIFSL | (SEQ ID NO:180) |
| FGNISIILVSHLDPQLDSPMYFFVSNLSFLDLCYTTSTVPQMLVNLRGPEKTISYGGCVA | |
| QLYIFLALGSTECILLAIMAFDRYAAICKPLHYPVIMNHRRCIHMAAGTWISGFANSLVQ | |
| STLTVVAPRCGQRVLDHFFCEVPALLKLACIDIRVNEMELNVLGALLLLMPLTLILGTYV | |
| FIAQAVMRICSAESRWKAFNTCASHLLVVSLFYFTAISMYVQPPSSYSHDRGKIIMALFY | |
| GIVTPTLNPFIYTLRNKDVKAALRRSLTKEFWIKTR | |

A search of sequence databases reveals that the NOV76 amino acid sequence has 197 of 308 amino acid residues (63%) identical to, and 245 of 308 amino acid residues (79%) similar to, the 313 amino acid residue ptnr:SPTREMBL-ACC:Q63394 protein from Rattus norvegicus (Rat) (OL1 RECEPTOR). Public amino acid databases include the GenBank databases, SwissProt, PDB and PIR.

The disclosed NOV76 polypeptide has homology to the amino acid sequences shown in the BLASTP data listed in Table 76C.

TABLE 76C
BLAST results for NOV76

| Gene Index/ | | Length | Identity | Positives | |
|---|---|---|---|---|---|
| Identifier | Protein/Organism | (aa) | (%) | (%) | Expect |
| gi\|17464349\|ref\|XP_ | similar to | 298 | 297/315 | 298/315 | e−147 |
| 069461.1\| | olfactory | | (94%) | (94%) | |
| (XM_069461) | receptor, family | | | | |
| | 2, subfamily B, | | | | |
| | member 2 [Homo | | | | |
| | sapiens] | | | | |
| gi\|18479350\|gb\|AAL60689.1\| | olfactory | 315 | 287/315 | 302/315 | e−147 |
| (AY073026) | receptor MOR256-3 | | (91%) | (95%) | |
| | [Mus musculus] | | | | |
| gi\|11177906\|ref\|NP_ | olfactory | 313 | 197/308 | 245/308 | e−104 |
| 068632.1\| | receptor [Mus | | (63%) | (78%) | |
| (NM_021860) | musculus] | | | | |
| gi\|14780900\|ref\|NP_ | olfactory | 357 | 199/307 | 245/307 | e−103 |
| 149046.1\| | receptor, family | | (64%) | (78%) | |
| (NM_033057) | 2, subfamily B, | | | | |
| | member 2 [Homo | | | | |
| | sapiens] | | | | |
| gi\|18480406\|gb\|AAL61217.1\| | olfactory | 313 | 196/308 | 242/308 | e−103 |
| (AY073554) | receptor MOR256- | | (63%) | (77%) | |
| | 10 [Mus musculus] | | | | |

Table 76D lists the domain descriptions from DOMAIN analysis results against NOV76. This indicates that the NOV76 sequence has properties similar to those of other proteins known to contain this domain.

TABLE 76D
Domain Analysis of NOV 76

gnl|Pfam|pfam00001, 7tm_1, 7 transmembrane receptor
(rhodopsin family).
CD-Length = 254 residues, 100.0% aligned
Score = 95.9 bits (237), Expect = 3e−21

The disclosed NOV76 nucleic acid of the invention encoding a GPCR-like protein includes the nucleic acid whose sequence is provided in Table 76A or a fragment thereof. The invention also includes a mutant or variant nucleic acid any of whose bases may be changed from the corresponding base shown in Table 76A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 33 percent of the bases may be so changed.

The disclosed NOV76 protein of the invention includes the GPCR-like protein whose sequence is provided in Table 76B. The invention also includes a mutant or variant protein any of whose residues may be changed from the corresponding residue shown in Table 76B while still encoding a protein that maintains its GPCR-like activities and physiological functions, or a functional fragment thereof. In the mutant or variant protein, up to about 37 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV76) may function as a member of a “GPCR family”. Therefore, the NOV76 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV76 nucleic acids and proteins of the invention are useful in potential therapeutic applications implicated in diseases including but not limited to various pathologies and disorders as indicated below. For example, a cDNA encoding the GPCR-like protein (NOV76) may be useful in gene therapy, and the GPCR-like protein (NOV76) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV76 nucleic acid encoding the GPCR-like protein of the invention, or fragments thereof, may further be useful in diagnostic applications, wherein the presence or amount of the nucleic acid or the protein are to be assessed.

NOV76 nucleic acids and polypeptides are further useful in the generation of antibodies that bind immuno-specifically to the novel NOV76 substances for use in therapeutic or diagnostic methods. These antibodies may be generated according to methods known in the art, using prediction from hydrophobicity charts, as described in the “Anti-NOVX Antibodies” section below. The disclosed NOV76 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV77

A disclosed NOV77 nucleic acid of 1014 nucleotides (also referred to as CG57419-01) encoding a GPCR-like protein is shown in Table 77A. The start and stop codons are in bold letters.

TABLE 77A
NOV77 nucleotide sequence.

(SEQ ID NO:181)

CATCTTTAGTGTGGTCCTTGGAAGCTCATGGGTCAGGAAAATAAAAACCAGACATCGGTGAGTGAGTTCA
TTCTGCTGGGGATTTCCAGTGATTGGGGCATTCAGGTATCCCTCTTCGCCCTGATCCTGGCCATGTATTT
GGTGACTATTTTAGGAAACACCCTCATTCTTCTTCTGATCAGACTGGACAACAGGCTTCATACCCCCATG
TACTTCTCCCTTAGTGTTCTGTCATTTGTGGACTTTTGTTATACAAAGAGTATTGTCCCACAAATGCTGT
CCCACTTGCTCTCAGCCCGAAAGTCCATCCCATTCTACAGTTGTGTGCTCCAGCTCTATGTTTCTCTGGC
ATTGTGTGGGTCTGAGTTCTTCCTGCTGGGGGCCATGGCCTATGACCGCTACGTGGCCGTGTGCCACCCA
CTGCACTACACGGTCATCATGCATGGAGGGCTGTGCCTGGGGCTGGCGGCCAGCCGCCTGGTGGCTGGCT
TCTCAAATTCCCTGATGGAAACAATTATCACCTTCCAGCTTTTATCACCTTCCAGCTTCCTGTGTCACGG
TGTTATCAATCACTTTGTCTGTGAGACCTTAGCAGTGCTACAGCTAGCCTGTGTGGATGTCCCCTTCAAC
AAGGTCATGGTGGCCATCTCAGGGTTTCTGGTGATCTTGCTTCCCTGTTCCCTGGTTCTATTCTCCTATG
CTTGCATAGTTGCCACCATTTTGTGCATTCGTTCTACCCAGGTACGCTGCAAAGCCTTTGGGACCTGTGC
CTCTCACCTCATTGTCGTTTGCATGTGCTTTGGGGCTACCATCTGCACCTACCTGGGGCCACAGTTGGCC
TCCTCAGCAGAGGAAGAGAAGATGATTGCTCTCTTCTATGGACTGGTGTCACCCATGTTGAACCCCTTGA
TCTACAGCTTGAGGAATAAGGAAGTTACGGCTGCTGTCCGGAAAGTTTTAGAAAGATGCAGATAAAGGGT
CAAGACTCTAAGAACCTCTTGTTATCTATCATCA

In a search of public sequence databases, the NOV77 nucleic acid sequence, located on chromsome 7 has 358 of 495 bases (72%) identical to a gb:GENBANK-ID:HSU56421|acc:U56421.1 mRNA from Homo sapiens (Human olfactory receptor (OLF3) gene, complete cds). Public nucleotide databases include all GenBank databases and the GeneSeq patent database.

The disclosed NOV77 polypeptide (SEQ ID NO:182) encoded by SEQ ID NO:181 has 315 amino acid residues and is presented in Table 77B using the one-letter amino acid code. Signal P, Psort and/or Hydropathy results predict that NOV77 has a signal peptide and is likely to be localized extracellularly with a certainty of 0.6000. The most likely cleavage site for a NOV77 peptide is between amino acids 43 and 44.

TABLE 77B
Encoded NOV77 protein sequence.

| | |
|---|---|
| MGQENKNQTWVSEFILLGISSDWGIQVSLFALILAMYLVTILGNTLILLLIRLDNRLHTP | (SEQ ID NO:182) |
| MYFSLSVLSFVDFCYTKSIVPQMLSHLLSARKSIPFYSCVLQLYVSLALCGSEFFLLGAM | |
| AYDRYVAVCHPLHYTVIMHGGLCLGLAASRLVAGFSNSLMETIITFQLLSPSSFLCHGVI | |
| NHFVCETLAVLQLACVDVPFNKVMVAISGFLVILLPCSLVLFSYACIVATILCIRSTQVR | |
| CKAFGTCASHLIVVCMCFGATICTYLGPQLASSAEEEKMIALFYGVVSPMLNPLIYSLRN | |
| KEVTAAVRKVLERCR | |

A search of sequence databases reveals that the NOV77 amino acid sequence has have 180 of 305 amino acid residues (59%) identical to, and 229 of 305 amino acid residues (75%) similar to, the 317 amino acid residue ptnr:SWISSPROT-ACC:Q95156 protein from Canis familiaris (Dog) (OLFACTORY RECEPTOR-LIKE PROTEIN OLF3). Public amino acid databases include the GenBank databases, SwissProt, PDB and PIR.

The disclosed NOV77 polypeptide has homology to the amino acid sequences shown in the BLASTP data listed in Table 77C.

TABLE 77C
BLAST results for NOV77

| Gene Index/ | | Length | Identity | Positives | |
|---|---|---|---|---|---|
| Identifier | Protein/Organism | (aa) | (%) | (%) | Expect |
| gi\|18480604\|gb\|AAL61316.1\| | olfactory | 310 | 239/315 | 268/315 | e−112 |
| (AY073653 | receptor MOR257-3 | | (75%) | (84%) | |
| | [Mus musculus] | | | | |
| gi\|2495055\|sp\|Q95156\| | OLFACTORY | 317 | 178/305 | 226/305 | E−82 |
| OLF3_CANFA | RECEPTOR-LIKE | | (58%) | (73%) | |
| | PROTEIN OLF3 | | | | |
| gi\|6912558\|ref\|NP_036501.1\| | olfactory | 317 | 173/305 | 221/305 | e−79 |
| (NM_012369) | receptor, family | | (56%) | (71%) | |
| | 2, subfamily F, | | | | |
| | member 1; | | | | |
| | olfactory | | | | |
| | receptor, family | | | | |
| | 2, subfamily F, | | | | |
| | member 5; | | | | |
| | olfactory | | | | |
| | receptor, family | | | | |
| | 2, subfamily F, | | | | |
| | member 4 [Homo | | | | |
| | sapiens] | | | | |
| gi\|9297120\|sp\|Q13607\| | OLFACTORY | 317 | 173/305 | 221/305 | e−79 |
| O2F1_HUMAN | RECEPTOR 2F1 | | (56%) | (71%) | |
| | (OLFACTORY | | | | |
| | RECEPTOR-LIKE | | | | |
| | PROTEIN OLF3) | | | | |
| gi\|18479500\|gb\|AAL60764.1\| | olfactory | 313 | 176/305 | 219/305 | 3e−79 |
| (AY073101) | receptor MOR257-1 | | (57%) | (71%) | |
| | [Mus musculus] | | | | |

Table 77D lists the domain descriptions from DOMAIN analysis results against NOV77. This indicates that the NOV77 sequence has properties similar to those of other proteins known to contain this domain.

TABLE 77D
Domain Analysis of NOV77

gnl|Pfam|pfam00001, 7tm_1, 7 transmembrane receptor
(rhodopsin family).
CD-Length = 254 residues, 94.1% aligned
Score = 75.5 bits (184), Expect = 4e−15

The disclosed NOV77 nucleic acid of the invention encoding a GPCR-like protein includes the nucleic acid whose sequence is provided in Table 77A or a fragment thereof. The invention also includes a mutant or variant nucleic acid any of whose bases may be changed from the corresponding base shown in Table 77A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 28 percent of the bases may be so changed.

The disclosed NOV77 protein of the invention includes the GPCR-like protein whose sequence is provided in Table 77B. The invention also includes a mutant or variant protein any of whose residues may be changed from the corresponding residue shown in Table 77B while still encoding a protein that maintains its GPCR-like activities and physiological functions, or a functional fragment thereof. In the mutant or variant protein, up to about 41 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV77) may function as a member of a “GPCR family”. Therefore, the NOV77 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV77 nucleic acids and proteins of the invention are useful in potential therapeutic applications implicated in diseases including but not limited to various pathologies and disorders as indicated below. For example, a cDNA encoding the GPCR-like protein (NOV77) may be useful in gene therapy, and the GPCR-like protein (NOV77) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV77 nucleic acid encoding the GPCR-like protein of the invention, or fragments thereof, may further be useful in diagnostic applications, wherein the presence or amount of the nucleic acid or the protein are to be assessed.

NOV77 nucleic acids and polypeptides are further useful in the generation of antibodies that bind immuno-specifically to the novel NOV77 substances for use in therapeutic or diagnostic methods. These antibodies may be generated according to methods known in the art, using prediction from hydrophobicity charts, as described in the “Anti-NOVX Antibodies” section below. The disclosed NOV77 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV78

A disclosed NOV78 nucleic acid of 1151 nucleotides (also referred to as CG57425-01) encoding a GPCR-like protein is shown in Table 78A. The start and stop codons are in bold letters.

TABLE 78A
NOV78 nucleotide sequence.

| | |
|---|---|
| GGACACTGGTTTGGGCCATATGGATGGTGAGCAATGTATGACCTGATTCTGTTCATTAAG | (SEQ ID NO:183) |
| ATAAGCTTTATGTCTCCTACTCTAAGAAACTCTTCAATTTTCATCATTCTCACCTTTTGC | |
| CTTTAGGTTTTCCGAAGGTCAACAATGAAAAACAGAACCATGTTTGGTGAGTTTATTCTA | |
| CTGGGCCTTACAAATCAACCTGAACTCCAAGTGATGATATTCATCTTTCTGTTCCTCACC | |
| TACATGCTAAGTGTCCTAGGAAATCTGACTATTATCACCCTCACCTTACTAGACCCCCAC | |
| CTCCAGACCCCCATGTATTTCTTCCTCCGGAATTTCTCCTTCTTAGAAATTTCCTTCACA | |
| TCCATTTTTATTCCCAGATTTCTGACCAGCATGACAACAGGAAATAAAGTTATCAGCTTT | |
| GCTGGCTGCTTGACTCAGTATTTTTTTGCTATATTTCTTGGAGCTACCGAGTTTTACCTC | |
| CTGGCCTCCATGTCTTATGATCGTTATGTGGCCATCTGCAAACCCTTGCATTACCTGACT | |
| ATTATGAGCAGCAGAGTCTGCATACAACTAGTGTTCTGCTCCTGGTTGGGGGGATTCCTA | |
| GCAATCTTACCACCAATCATCCTGATGACCCAGGTAGATTTCTGTGTCTCCAACATTCTG | |
| AATCACTATTACTGTGACTATGGGCCTCTCGTGGAGCTTGCCTGCTCAGACACAAGCCTC | |
| TTAGAACTGATGGTCATCCTCTTGGCCGTTGTGACTCTCATGGTTACTCTCGTGCTGGTG | |
| ACACTTTCTTACACATACATTATCAGGACTATTCTGAGGATCCCTTCCGCCCAGCAAAGG | |
| ACAAAGGCCTTTTCCACTTGTTCCTCCCACATGATTGTCATCTCCCTCTCTTATGGCAGC | |
| TGCATGTTTATGTACATTAATCCTTCTGCAAAAGAAGGAGGTGCTTTCAACAAAGGAATA | |
| GCTGTACTCATTACTTCGGTTACTCCCTTACTGAATCCCTTCATATATACTTTAAGAAAT | |
| CAGCAAGTGAAACAAGCTTTCAAGGACTCAGTCAAAAAGATTGTGAAACTTTAAAAAAGG | |
| AGATTACACTTCAAAATACATTTTCACTTAACAAATATGCATTGAATGTCTATATTTCAA | |
| GTGCTAAATTG | |

In a search of public sequence databases, the NOV78 nuceic acid sequence, located on chromsome 12 has 605 of 931 bases (64%) identical to a gb:GENBANK-ID:AF102523|acc:AF102523.1 mRNA from Mus musculus (Mus musculus olfactory receptor C6 gene, complete cds). Public nucleotide databases include all GenBank databases and the GeneSeq patent database.

The disclosed NOV78 polypeptide (SEQ ID NO:184) encoded by SEQ ID NO:183 has 309 amino acid residues and is presented in Table 78B using the one-letter amino acid code. Signal P, Psort and/or Hydropathy results predict that NOV78 has a signal peptide and is likely to be localized at the plasma membrane with a certainty of 0.6000. The most likely cleavage site for a NOV78 peptide is at amino acid 39.

TABLE 78B
Encoded NOV78 protein sequence.

| | |
|---|---|
| MKNRTMFGEFILLGLTNQPELQVMIFIFLFLTYMLSVLGNLTIITLTLLDPHLQTPMYFF | (SEQ ID NO:184) |
| LRNFSFLEISFTSIFIPRFLTSMTTGNKVISFAGCLTQYFFAIFLGATEFYLLASMSYDR | |
| YVAICKPLHYLTTMSSRVCIQLVFCSWLGGFLAILPPIILMTQVDFCVSNILNHYYCDYG | |
| PLVELACSDTSLLELMVILLAVVTLMVTLVLVTLSYTYIIRTILRIPSAQQRTKAFSTCS | |
| SHMIVISLSYGSCMFMYINPSAKEGGAFNKGIAVLITSVTPLLNPFIYTLRNQQVKQAFK | |
| DSVKKIVKL | |

A search of sequence databases reveals that the NOV78 amino acid sequence has 175 of 309 amino acid residues (56%) identical to, and 222 of 309 amino acid residues (71%) similar to, the 313 amino acid residue ptnr:SPTREMBL-ACC:Q9Z1V0 protein from Mus musculus (Mouse) (OLFACTORY RECEPTOR C6)(. Public amino acid databases include the GenBank databases, SwissProt, PDB and PIR.

NOV78 is expressed in at least Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV78 polypeptide has homology to the amino acid sequences shown in the BLASTP data listed in Table 78C.

TABLE 78C
BLAST results for NOV78

| Gene Index/ | | Length | Identity | Positives | |
|---|---|---|---|---|---|
| Identifier | Protein/Organism | (aa) | (%) | (%) | Expect |
| gi\|17474307\|ref\|XP_ | similar to | 309 | 308/309 | 309/309 | e−141 |
| 062466.1\| | olfactory | | (99%) | (99%) | |
| (XM_062466) | receptor 49 [Homo | | | | |
| | sapiens] | | | | |
| gi\|18480848\|gb\|AAL61438.1\| | olfactory | 309 | 238/309 | 238/309 | e−114 |
| (AY073775) | receptor MOR115-4 | | (77%), | (77%), | |
| | [Mus musculus] | | Positives = | Positives = | |
| | | | 273/309 | 273/309 | |
| | | | (88%) | (88%) | |
| gi\|18479958\|gb\|AAL60993.1\| | olfactory | 309 | 239/309 | 239/309 | e−112 |
| (AY073330) | receptor MOR115-1 | | (77%), | (77%), | |
| | [Mus musculus] | | Positives = | Positives = | |
| | | | 274/309 | 274/309 | |
| | | | (88%) | (88%) | |
| gi\|17474309\|ref\|XP_ | similar to | 309 | 247/309 | 247/309 | e−112 |
| 062467.1\| | olfactory | | (79%), | (79%), | |
| (XM_062467) | receptor 49 [Homo | | Positives = | Positives = | |
| | sapiens] | | 278/309 | 278/309 | |
| | | | (89%) | (89%) | |
| gi\|18479614\|gb\|AAL60821.1\| | olfactory | 312 | 207/306 | 207/306 | e−110 |
| (AY073158) | receptor MOR114-1 | | (67%), | (67%), | |
| | [Mus musculus] | | Positives = | Positives = | |
| | | | 256/306 | 256/306 | |
| | | | (83%) | (83%) | |

Table 78D lists the domain descriptions from DOMAIN analysis results against NOV78. This indicates that the NOV78 sequence has properties similar to those of other proteins known to contain this domain.

TABLE 78D
Domain Analysis of NOV78

gnl|Pfam|pfam00001, 7tm_1, 7 transmembrane receptor
(rhodopsin family).
CD-Length = 254 residues, 100.0% aligned
Score = 90.1 bits (222), Expect = 2e−19

G-Protein Coupled Receptor (GPCRs) have been identified as an extremely large family of protein receptors in a number of species. At the phylogenetic level they can be classified into four major subfamilies. These receptors share a seven transmembrane domain structure with many neurotransmitter and hormone receptors. They are likely to be involved in the recognition and transduction of various signals mediated by G-Proteins, hence their name G-Protein Coupled Receptors. The human GPCR genes are generally intron-less and belong to four gene subfamilies, displaying great sequence variability. These genes are dominantly expressed in olfactory epithelium.

Olfactory receptors (ORs) have been identified as extremely large family of GPCRs in a number of species. As members of the GPCR family, these receptors share a seven transmembrane domain structure with many neurotransmitter and hormone receptors, and are likely to underlie the recognition and G-protein-mediated transduction of odorant signals. Like GPCRs, the ORs they can be expressed in a variety of tissues where they are thought to be involved in recognition and transmission of a variety of signals. The human OR genes are typically intron-less and belong to four different gene subfamilies, displaying great sequence variability. These genes are dominantly expressed in olfactory epithelium.

The disclosed NOV78 nucleic acid of the invention encoding a GPCR-like protein includes the nucleic acid whose sequence is provided in Table 78A or a fragment thereof. The invention also includes a mutant or variant nucleic acid any of whose bases may be changed from the corresponding base shown in Table 78A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36 percent of the bases may be so changed.

The disclosed NOV78 protein of the invention includes the GPCR-like protein whose sequence is provided in Table 78B. The invention also includes a mutant or variant protein any of whose residues may be changed from the corresponding residue shown in Table 78B while still encoding a protein that maintains its GPCR-like activities and physiological functions, or a functional fragment thereof. In the mutant or variant protein, up to about 44 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV78) may function as a member of a “GPCR family”. Therefore, the NOV78 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV78 nucleic acids and proteins of the invention are useful in potential therapeutic applications implicated in diseases including but not limited to various pathologies and disorders as indicated below. For example, a cDNA encoding the GPCR-like protein (NOV78) may be useful in gene therapy, and the GPCR-like protein (NOV78) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV78 nucleic acid encoding the GPCR-like protein of the invention, or fragments thereof, may further be useful in diagnostic applications, wherein the presence or amount of the nucleic acid or the protein are to be assessed.

NOV78 nucleic acids and polypeptides are further useful in the generation of antibodies that bind immuno-specifically to the novel NOV78 substances for use in therapeutic or diagnostic methods. These antibodies may be generated according to methods known in the art, using prediction from hydrophobicity charts, as described in the “Anti-NOVX Antibodies” section below. The disclosed NOV78 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV79

A disclosed NOV79 nucleic acid of 1601 nucleotides (also referred to as CG57753-01) encoding a GPCR-like protein is shown in Table 79A. The start and stop codons are in bold letters.

TABLE 79A
NOV79 nucleotide sequence.

| | |
|---|---|
| CTGGTTCTTTGAGTGAGTTATTCCTGGATTCTAGGAGCTCACAGTAGAGTGTTTCAGAAT | (SEQ ID NO:185) |
| GGCAAATATCTAAACATTAGCCGGTAATTTTATGCTCCGTATACTGGGTACTAATTTACA | |
| TAAACATATAAGTAAAGTCTACACATATGAGACTGTTTTCTTGATAGATCATGGAAGGAA | |
| AAATCCATTCAGGGAAAAAAAAGGGAAATACTATATAAATGTCAAAAATCCAGTCTTTTT | |
| AAGAGACATTCTCTGGAAATATCTCTATTTTGAGGTGTAGTAGATTATCTTACATATATA | |
| TCCACTCACACATACCTTCCAGTTAGAACACTGAAGCCTCATCATTGTAATTAAAGCAAT | |
| AAATTTTGTAAAAATGAAAAGGATAATTGTGGGAGGAGATTCTAAACACTCCTTTTCTAA | |
| TGAGCTGCTCTGTGTCGCCAGGGGAAACATGGTTGAGTAAGGCATCACATTTTTGACATG | |
| GAGCTTCTGACAAATAATCTCAAATTTATCATTGACCCTTTTGTTTACAGGTTCTGACAC | |
| CTTAGTCCAATACCTTCAGAAGAACACATGGAAAATAGGAAAAATTGACTTAATTCATCC | |
| TCTTGGGGCTCACACAGAACCCTGAGGGCCAAAAAGTTTTATTTGTCACATTCTTACTCA | |
| TCTACATTGTGACGATAATGGGCAACCTCCTTATCATGGTGACCATCATGGCCAGCCAGT | |
| CCCTGGGTTCCCCCATGTACTTTTTTCTGGCTTCTTTATCATTTATACATACCGTCTATT | |
| ATACTGCCATTGCTCCCAAAATGATTGTTGACCTGCTCTCTGAGAAAAAGACCATTTCTT | |
| TTCAGGGTTGTATGGCTCAACTTTTTATGGATCATTTATTTGCTGGTGCTGAGGTCATTC | |
| TTCTGGTGGTAATGGCCTATGATCAATATGTGGCCATCTGTAAGCCTCTTCATTATTTGA | |
| TCATCATGAATCGTCGAGTCTGTGTTCTCATGCTGTTGGTGGCCTGGATTGGAGGCTTTC | |
| TTCACTCATTGGTTCAATTTCTCTTTATTTATCAGCTCCCTTTCTGTGGACCCAATGTCA | |
| TTGACAACTTCCTGTGTGATTTGTATCCCTTATTGAAACTTGCTTGCACCAATACCTATG | |
| TCACTGGGCTTTCTATGATAGCTAATGGTGGAGCGATTTGTACTGTCACCTTCTTCCCTC | |
| TCCTGCTTTCCTATGGGGTCATATTACCCTCTCTTAAGACTCAGAGTTTGGAAGGGAAAT | |
| GCAAAGCTTTCTACACCTGTGCATCCCACATCACTGTGATCACTTTATTCTTTGTCCCCT | |
| GCATCTTCCTGTTTGTAAGCCCCAACTCCACCTTTCCCATTGATAAATCCATGACTGTGG | |
| TTTTAACTTGTATAACTCCCATGCTGAAACCACTAATCTATGCCCTGAGGAATGCAGAAA | |
| TGAAAAGTGCCATGAGGAAACTTTGGAGTGAAAAAGTAAGCTTAGCTGGAAAAGGGCTGT | |
| ATCCCTCATGAGAATATGACTTTCATTCTTTCACAGAAGCAAGGAATAATTTCACTATCC | |
| TATCAGATTACATTTCTGTTATCATTCGCCTTTAGTTATTT | |

In a search of public sequence databases, the NOV79 nucleic acid sequence, located on chromsome 12 has 605 of 931 bases (64%) identical to a gb:GENBANK-ID:AF102523|acc:AF102523.1 mRNA from Mus musculus (Mus musculus olfactory receptor C6 gene, complete cds). Public nucleotide databases include all GenBank databases and the GeneSeq patent database.

The disclosed NOV79 polypeptide (SEQ ID NO:186) encoded by SEQ ID NO:185 has 277 amino acid residues and is presented in Table 79B using the one-letter amino acid code. Signal P, Psort and/or Hydropathy results predict that NOV79 has a signal peptide and is likely to be localized at the plasma membrane with a certainty of 0.6000. The most likely cleavage site for a NOV79 peptide is at amino acid position 39.

TABLE 79B
Encoded NOV79 protein sequence.

| | |
|---|---|
| MGNLLIMVTIMASQSLGSPMYFFLASLSFIHTVYYTAIAPKMIVDLLSEKKTISFQGCMA | (SEQ ID NO:186) |
| QLFMDHLFAGAEVILLVVMAYDQYVAICKPLHYLIIMNRRVCVLMLLVAWIGGFLHSLVQ | |
| FLFIYQLPFCGPNVIDNFLCDLYPLLKLACTNTYVTGLSMIANGGAICTVTFFPLLLSYG | |
| VILPSLKTQSLEGKCKAFYTCASHITVITLFFVPCIFLFVRPNSTFPIDKSMTVVLTCIT | |
| PMLKPLIYALRNAEMKSAMRKLWSEKVSLAGKGLYPS | |

A search of sequence databases reveals that the NOV79 amino acid sequence has 175 of 309 amino acid residues (56%) identical to, and 222 of 309 amino acid residues (71%) similar to, the 313 amino acid residue ptnr:SPTREMBL-ACC:Q9Z1V0 protein from Mus musculus (Mouse) (OLFACTORY RECEPTOR C6)(. Public amino acid databases include the GenBank databases, SwissProt, PDB and PIR.

NOV79 is expressed in at least Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV79 polypeptide has homology to the amino acid sequences shown in the BLASTP data listed in Table 79C.

TABLE 79C
BLAST results for NOV79

| Gene Index/ | | Length | Identity | Positives | |
|---|---|---|---|---|---|
| Identifier | Protein/Organism | (aa) | (%) | (%) | Expect |
| gi\|17459952\|ref\|XP_ | similar to | 277 | 275/277 | 276/277 | e−133 |
| 062090.1\| | odorant receptor | | (99%) | (99%) | |
| (XM_062090) | 16 [Homo sapiens] | | | | |
| gi\|17460091\|ref\|XP_ | similar to | 277 | 275/277 | 275/277 | e−132 |
| 062159.1\| | odorant receptor | | (99%) | (99%) | |
| (XM_062159) | 16 [Homo | | | | |
| | sapiens] | | | | |
| gi\|17460099\|ref\|XP_ | similar to | 722 | 242/266 | 251/266 | e−119 |
| 062161.1\| | odorant receptor | | (90%) | (93%) | |
| (XM_062161) | 16 [Homo sapiens] | | | | |
| gi\|18479528\|gb\|AAL60778.1\| | olfactory | 314 | 226/277 | 247/277 | e−109 |
| (AY073115) | receptor MOR231-2 | | (81%) | (88%) | |
| | [Mus musculus] | | | | |
| gi\|18479534\|gb\|AAL60781.1\| | olfactory | 305 | 186/265 | 223/265 | 7e−94 |
| (AY073118) | receptor MOR231-3 | | (70%) | (83%) | |
| | [Mus musculus] | | | | |

Table 79D lists the domain descriptions from DOMAIN analysis results against NOV79. This indicates that the NOV79 sequence has properties similar to those of other proteins known to contain this domain.

TABLE 79D
Domain Analysis of NOV79

gnl|Pfam|pfam00001, 7tm_1, 7 transmembrane receptor
(rhodopsin family).
CD-Length = 254 residues, 100.0% aligned
Score = 91.3 bits (225), Expect = 7e−20

G-Protein Coupled Receptor (GPCRs) have been identified as an extremely large family of protein receptors in a number of species. At the phylogenetic level they can be classified into four major subfamilies. These receptors share a seven transmembrane domain structure with many neurotransmitter and hormone receptors. They are likely to be involved in the recognition and transduction of various signals mediated by G-Proteins, hence their name G-Protein Coupled Receptors. The human GPCR genes are generally intron-less and belong to four gene subfamilies, displaying great sequence variability. These genes are dominantly expressed in olfactory epithelium.

Olfactory receptors (ORs) have been identified as extremely large family of GPCRs in a number of species. As members of the GPCR family, these receptors share a seven transmembrane domain structure with many neurotransmitter and hormone receptors, and are likely to underlie the recognition and G-protein-mediated transduction of odorant signals. Like GPCRs, the ORs they can be expressed in a variety of tissues where they are thought to be involved in recognition and transmission of a variety of signals. The human OR genes are typically intron-less and belong to four different gene subfamilies, displaying great sequence variability. These genes are dominantly expressed in olfactory epithelium. The disclosed NOV79 nucleic acid of the invention encoding a GPCR-like protein includes the nucleic acid whose sequence is provided in Table 79A or a fragment thereof. The invention also includes a mutant or variant nucleic acid any of whose bases may be changed from the corresponding base shown in Table 79A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 36 percent of the bases may be so changed.

The disclosed NOV79 protein of the invention includes the GPCR-like protein whose sequence is provided in Table 79B. The invention also includes a mutant or variant protein any of whose residues may be changed from the corresponding residue shown in Table 79B while still encoding a protein that maintains its GPCR-like activities and physiological functions, or a functional fragment thereof. In the mutant or variant protein, up to about 44 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV79) may function as a member of a “GPCR family”. Therefore, the NOV79 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV79 nucleic acids and proteins of the invention are useful in potential therapeutic applications implicated in diseases including but not limited to various pathologies and disorders as indicated below. For example, a cDNA encoding the GPCR-like protein (NOV79) may be useful in gene therapy, and the GPCR-like protein (NOV79) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV79 nucleic acid encoding the GPCR-like protein of the invention, or fragments thereof, may further be useful in diagnostic applications, wherein the presence or amount of the nucleic acid or the protein are to be assessed.

NOV79 nucleic acids and polypeptides are further useful in the generation of antibodies that bind immuno-specifically to the novel NOV79 substances for use in therapeutic or diagnostic methods. These antibodies may be generated according to methods known in the art, using prediction from hydrophobicity charts, as described in the “Anti-NOVX Antibodies” section below. The disclosed NOV79 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV80

A disclosed NOV80 nucleic acid of 1006 nucleotides (also referred to as CG56766-01) encoding a GPCR-like protein is shown in Table 80A. The start and stop codons are in bold letters.

TABLE 80A
NOV80 nucleotide sequence.

(SEQ ID NO:187)

CCCTTTCCTCTTGCTCTTTGATGTTTTGTAGGCCTGCAGCTCCCAAGCACAGAGGCATGAGTGGGGAGAA
TGTCACCAAGGTCAGCACCTTCATCCTGGTGGGCCTCCCCACGGCCCCAGGGCTGCAGTACCTGCTCTTC
CTCCTCTTCCTGCTCACCTACCTCTTTGTCCTGGTGGAGAACCTGGCCATCATCCTCATCGTCTGGAGCA
GCACCTCCCTCCACAGGCCCATGTACTACTTTCTGAGCTCCATGTCTTTCCTGGAGATCTGGTACGTGTC
TGACATCACCCCCAAGATGCTGGAGGGCTTCCTCCTCCAGCAGAAACGCATCTCTTTCGTCGGGTGCATG
ACGCAGCTCTACTTCTTCAGCTCCCTGGTGTGCACCGAGTGTGTGCTTCTGGCCTCCATGGCCTACGACC
GCTACGTGGCCATCTGCCACCCGCTGCGCTACCACGTCCTTGTGACCCCGGGGCTGTGCCTCCAGCTGGT
GGGCTTCTCCTTTGTGAGTGGCTTCACCATCTCCATGATCAAGGTCTGTTTTATCTCCAGCGTCACGTTC
TGTGGCTCCAACGTCTTGAACCACTTCTTCTGTGACATTTCCCCCATCCTCAAGCTGGCCTGCACGGACT
TCTCCACTGCAGAGCTGGTGGATTTCATCCTGGCCTTCATCATCCTGCTGTTTCCGCTCCTGGCCACCAT
ACTGTCATATTGGCACATCACCCTGGCTGTCCTGCGCATCCCCTCGGCCACCGGCTGCTGGAGAGCCTTC
TCTACCTGCGCCTCTCACCTCACCGTGGTCACCCTCTTCTATACAGCCTTGCTTTTCATGTATGTCCGGC
CCCAAGCCATTGATTCCCAGAGCTCCAACAAGCTCATCTCTGCCGTGTACACTGTTGTCACGCCAATAAT
TAACCCTTTGATTTACTGCCTGAGGAACAAGGAATTTAAGGACGCCTTGAAAAAGGCCTTGGGCTTGGGT
CAAACTTCACACTAAGACAACTAAAT

The disclosed NOV80 polypeptide (SEQ ID NO:188) encoded by SEQ ID NO:187 has 324 amino acid residues and is presented in Table 80B using the one-letter amino acid code.

TABLE 80B
Encoded NOV80 protein sequence.

(SEQ ID NO:188)

MFCRPAAPKHRGMSGENVTKVSTFILVGLPTAPGLQYLLFLLFLLTYLFVLVENLAIILIVWSSTSLHRP
MYYFLSSMSFLEIWYVSDITPKMLEGFLLQQKRISFVGCMTQLYFFSSLVCTECVLLASMAYDRYVAICH
PLRYHVLVTPGLCLQLVGFSFVSGFTISMIKVCFISSVTFCGSNVLNHFFCDISPILKLACTDFSTAELV
DFILAFIILVFPLLATILSYWHITLAVLRIPSATGCWRAFSTCASHLTVVTVFYTALLFMYVRPQAIDSQ
SSNKLISAVYTVVTPIINPLIYCLRNKEFKDALKKALGLGQTSH

A search of sequence databases reveals that the NOV80 amino acid sequence has 215/305 (70%) identity and 253/305 (82%) similarity with TPEMBLNEW-ACC:AAG45189 M51 OLFACTORY RECEPTOR—Mus musculus. Public amino acid databases include the GenBank databases, SwissProt, PDB and PIR.

The disclosed NOV80 polypeptide has homology to the amino acid sequences shown in the BLASTP data listed in Table 80C.

TABLE 80C
BLAST results for NOV80

| Gene Index/ | | Length | Identity | Positives | |
|---|---|---|---|---|---|
| Identifier | Protein/Organism | (aa) | (%) | (%) | Expect |
| gi\|17435888\|ref\|XP_ | similar to | 312 | 312/312 | 312/312 | e−142 |
| 065377.1\| | olfactory | | (100%) | (100%) | |
| (XM_065377) | receptor 41 [Homo | | | | |
| | sapiens] | | | | |
| gi\|17435880\|ref\|XP_ | similar to | 331 | 293/307 | 300/307 | e−132 |
| 065375.1\| | olfactory | | (95%) | (97%) | |
| (XM_065375) | receptor 41 [Homo | | | | |
| | sapiens] | | | | |
| gi\|18479396\|gb\|AAL60712.1\| | olfactory | 312 | 271/309 | 289/309 | e−123 |
| (AY073049) | receptor MOR103-2 | | (87%) | (92%) | |
| | [Mus musculus] | | | | |
| gi\|18479398\|gb\|AAL60713.1\| | olfactory | 312 | 268/310 | 288/310 | e−123 |
| (AY073050) | receptor MOR103-3 | | (86%) | (92%) | |
| | [Mus musculus] | | | | |
| gi\|12007416\|gb\|AAG45189.1\| | m51 olfactory | 314 | 215/305 | 253/305 | e−102 |
| (AF321234) | receptor [Mus | | (70%) | (82%) | |
| | musculus] | | | | |

Table 80D lists the domain descriptions from DOMAIN analysis results against NOV80. This indicates that the NOV80 sequence has properties similar to those of other proteins known to contain this domain.

TABLE 80D
Domain Analysis of NOV80

gnl|Pfam|pfam00001, 7tm_1, 7 transmembrane receptor
(rhodopsin family).
CD-Length = 254 residues, 100.0% aligned
Score = 98.6 bits (244), Expect = 5e−22

The disclosed NOV80 nucleic acid of the invention encoding a GPCR-like protein includes the nucleic acid whose sequence is provided in Table 80A or a fragment thereof. The invention also includes a mutant or variant nucleic acid any of whose bases may be changed from the corresponding base shown in Table 80A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV80 protein of the invention includes the GPCR-like protein whose sequence is provided in Table 80B. The invention also includes a mutant or variant protein any of whose residues may be changed from the corresponding residue shown in Table 80B while still encoding a protein that maintains its GPCR-like activities and physiological functions, or a functional fragment thereof. In the mutant or variant protein, up to about 30 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV80) may function as a member of a “GPCR family”. Therefore, the NOV80 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV80 nucleic acids and proteins of the invention are useful in potential therapeutic applications implicated in diseases including but not limited to various pathologies and disorders as indicated below. For example, a cDNA encoding the GPCR-like protein (NOV80) may be useful in gene therapy, and the GPCR-like protein (NOV80) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV80 nucleic acid encoding the GPCR-like protein of the invention, or fragments thereof, may further be useful in diagnostic applications, wherein the presence or amount of the nucleic acid or the protein are to be assessed.

NOV80 nucleic acids and polypeptides are further useful in the generation of antibodies that bind immuno-specifically to the novel NOV80 substances for use in therapeutic or diagnostic methods. These antibodies may be generated according to methods known in the art, using prediction from hydrophobicity charts, as described in the “Anti-NOVX Antibodies” section below. The disclosed NOV80 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV81

NOV81 includes two GPCR-like proteins disclosed below. The disclosed sequences have been named NOV81a and NOV81b.

NOV81a

A disclosed NOV81a nucleic acid of 1039 nucleotides (also referred to as CG57847-01) encoding a GPCR-like protein is shown in Table 81A. The start and stop codons are in bold letters.

TABLE 81A
NOV81a nucleotide sequence.

(SEQ ID NO:189)

GAATGATGCCCTTTTGCCACAATATAATTAATATTTCCTGTGTGAAAAACAACTGGTCAAATGATGTCCG
TGCTTCCCTGTACAGTTTAATGGTGCTCATAATTCTGACCACACTCGTTGGCAATCTGATAGTTATTGTT
TCTATATCACACTTCAAACAACTTCATACCCCAACAAATTGGCTCATTCATTCCATGGCCACTGTGGACT
TTCTTCTGGGGTGTCTGGTCATGCCTTACAGTATGGTGAGATCTGCTGAGCACTGTTGGTATTTTGGAGA
AGTCTTCTGTAAAATTCACACAAGCACCGACATTATGCTGAGCTCAGCCTCCATTTTCCATTTGTCTTTC
ATCTCCATTGACCGCTACTATGCTGTGTGTGATCCACTGAGATATAAAGCCAAGATGAATATCTTGGTTA
TTTGTGTGATGATCTTCATTAGTTGGAGTGTCCCTGCTGTTTTTGCATTTGGAATGATCTTTCTGGAGCT
AAACTTCAAAGGCGCTGAAGAGATATATTACAAACATGTTCACTGCAGAGGAGGTTGCTCTGTCTTCTTT
AGCAAAATATCTGGGGTACTGACCTTTATGACTTCTTTTTATATACCTGGATCTATTATGTTATGTGTCT
ATTACAGAATATATCTTATCGCTAAAGAACAGGCAAGATTAATTAGTGATGCCAATCAGAAGCTCCAAAT
TGGATTGGAAATGAAAAATGGAATTTCACAAAGCAAAGAAAGGAAAGCTGTGAAGACATTGGGGATTGTG
ATGGGAGTTTTCCTAATATGCTGGTGCCCTTTCTTTATCTGTACAGTCATGGACCCTTTTCTTCACTACA
TTATTCCACCTACTTTGAATGATGTATTGATTTGGTTTGGCTACTTGAACTCTACATTTAATCCAATGGT
TTATGCATTTTTCTATCCTTGGTTTAGAAAAGCACTGAAGATGATGCTGTTTGGTAAAATTTTCCAAAAA
GATTCATCCAGGTGTAAATTATTTTTGGAATTGAGTTCATAGAATTATTATATTTTACT

The disclosed NOV81a polypeptide (SEQ ID NO:190) encoded by SEQ ID NO:189 has 339 amino acid residues and is presented in Table 81B using the one-letter amino acid code.

TABLE 81B
Encoded NOV81a protein sequence.

(SEQ ID NO:190)

MMPFCHNIINISCVKNNWSNDVRASLYSLMVLIILTTLVGNLIVIVSISHFKQLHTPTNWLIHSMATVDF
LLGCLVMPYSMVRSAEHCWYFGEVFCKIHTSTDIMLSSASIFHLSFISIDRYYAVCDPLRYKAKMNILVI
CVMIFISWSVPAVFAFGMIFLELNFKGAEEIYYKHVHCRGGCSVFFSKISGVLTFMTSFYIPGSIMLCVY
YRIYLIAKEQARLISDANQKLQIGLEMKNGISQSKERKAVKTLGIVMGVFLICWCPFFICTVMDPFLHYI
IPPTLNDVLIWFGYLNSTFNPMVYAFFYPWFRKALKMMLFGKIFQKDSSRCKLFLELSS

A search of sequence databases reveals that the NOV81a amino acid sequence has 152/299 (50%) identity and 206/299 (68%) similarity to SPTREMBL-ACC:Q9PIP4G PROTEIN-COUPLED RECEPTOR 57—Homo sapiens. Public amino acid databases include the GenBank databases, SwissProt, PDB and PIR.

NOV81b

A disclosed NOV81b nucleic acid of 1039 nucleotides (also referred to as CG57847-02) encoding a GPCR-like protein is shown in Table 81C. The start and stop codons are in bold letters.

TABLE 81C
NOV81b nucleotide sequence.

| | |
|---|---|
| GAATGATGCCCTTTTGCCACAATATAATTAATATTTCCTGTGTGAAAAACAACTGGTCAA | (SEQ ID NO:191) |
| ATGATGTCCGTGCTTCCCTGTACAGTTTAATGGTGCTCATAATTCTGACCACACTCGTTG | |
| GCAATCTGATAGTTATTGTTTCTATATCACACTTCAAACAACTTCATACCCCAACAAATT | |
| GGCTCATTCATTCCATGGCCACTGTGGACTTTCTTCTGGGGTGTCTGGTCATGCCTTACA | |
| GTATGGTGAGATCTGCTGAGCACTGTTGGTATTTTGGAGAAGTCTTCTGTAAAATTCACA | |
| CAAGCACCGACATTATGCTGAGCTCAGCCTCCATTTTCCATTTGTCTTTCATCTCCATTG | |
| ACCGCTACTATGCTGTGTGTGATCCACTGAGATATAAAGCCAAGATGAATATCTTGGTTA | |
| TTTGTGTGATGATCTTCATTAGTTGGAGTGTCCCTGCTGTTTTTGCATTTGGAATGATCT | |
| TTCTGGAGCTAAACTTCAAAGGCGCTGAAGAGATATATTACAAACATGTTCACTGCAGAG | |
| GAGGTTGCTCTGTCTTCTTTAGCAAAATATCTGGGGTACTGACCTTTATGACTTCTTTTT | |
| ATATACCTGGATCTATTATGTTATGTGTCTATTACAGAATATATCTTATCGCTAAAGAAC | |
| AGGCAAGATTAATTAGTGATGCCAATCAGAAGCTCCAAATTGGATTGGAAATGAAAAATG | |
| GAATTTCACAAAGCAAAGAAAGGAAAGCTGTGAAGACATTGGGGATTGTGATGGGAGTTT | |
| TCCTAATATGCTGGTGCCCTTTCTTTATCTGTACAGTCATGGACCCTTTTCTTCACTACA | |
| TTATTCCACCTACTTTGAATGATGTATTGATTTGGTTTGGCTACTTGAACTCTACATTTA | |
| ATCCAATGGTTTATGCATTTTTCTATCCTTGGTTTAGAAAAGCACTGAAGATGATGCTGT | |
| TTGGTAAAATTTTCCAAAAAGATTCATCCAGGTGTAAATTATTTTTGGAATTGAGTTCAT | |
| AGAATTATTATATTTTACT | |

In a search of public sequence databases, the NOV81b nucleic acid sequence, located on chromsome 6 has 616 of 979 bases (62%) identical to a gb:GENBANK-ID:HSU88828|acc:U88828.1 mRNA from Homo sapiens (Homo sapiens serotonin-4-receptor-like pseudogene). Public nucleotide databases include all GenBank databases and the GeneSeq patent database.

The disclosed NOV81b polypeptide (SEQ ID NO:192) encoded by SEQ ID NO:191 has 339 amino acid residues and is presented in Table B using the one-letter amino acid code. Signal P, Psort and/or Hydropathy results predict that NOV81b has a signal peptide and is likely to be localized in the plasma membrane with a certainty of 0.6000. The most likely cleavage site for a NOV81b peptide is between amino acids 47 and 48.

TABLE 81D
Encoded NOV81b protein sequence.

| | |
|---|---|
| MMPFCHNIINISCVKNNWSNDVRASLYSLMVLIILTTLVGNLIVIVSISHFKQLHTPTNW | (SEQ ID NO:192) |
| LIHSMATVDFLLGCLVMPYSMVRSAEHCWYFGEVFCKIHTSTDIMLSSASIFHLSFISID | |
| RYYAVCDPLRYKAKMNILVICVMIFISWSVPAVFAFGMIFLELNFKGAEEIYYKHVHCRG | |
| GCSVFFSKISGVLTFMTSFYIPGSIMLCVYYRIYLIAKEQARLISDANQKLQIGLEMKNG | |
| ISQSKERKAVKTLGIVMGVFLICWCPFFICTVMDPFLHYIIPPTLNDVLIWFGYLNSTEN | |
| PMVYAFFYPWFRKALKMMLFGKIFQKDSSRCKLFLELSS | |

A search of sequence databases reveals that the NOV81b amino acid sequence has 152 of 299 amino acid residues (50%) identical to, and 206 of 299 amino acid residues (68%) similar to, the 343 amino acid residue ptnr:SPTREMBL-ACC:Q9PIP4 protein from Homo sapiens (Human) (G PROTEIN-COUPLED RECEPTOR 57). Public amino acid databases include the GenBank databases, SwissProt, PDB and PIR.

NOV81b is expressed in at least Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV81b polypeptide has homology to the amino acid sequences shown in the BLASTP data listed in Table 81E.

TABLE 81E
BLAST results for NOV81b

| Gene Index/ | | Length | Identity | Positives | |
|---|---|---|---|---|---|
| Identifier | Protein/Organism | (aa) | (%) | (%) | Expect |
| gi\|10441577\|gb\|AAG17112.1\| | putative | 339 | 339/339 | 339/339 | e−177 |
| AF200627_1 | catecholamine | | (100%) | (100%) | |
| (AF200627) | receptor [Homo | | | | |
| | sapiens] | | | | |
| gi\|17453976\|ref\|XP_ | similar to trace | 338 | 338/338 | 338/338 | e−176 |
| 069048.1\| | amine receptor 1 | | (100%) | (100%) | |
| (XM_069048) | [Homo sapiens] | | | | |
| gi\|14600076\|gb\|AAK71237.1\| | trace amine | 332 | 261/334 | 261/334 | e−136 |
| AF380186_1 | receptor 1 | | (78%), | (78%), | |
| (AF380186) | [Rattus | | Positives = | Positives = | |
| | norvegicus] | | 288/334 | 288/334 | |
| | | | (86%), | (86%), | |
| gi\|18182341\|gb\|AAL65137.1\| | trace amine | 332 | 261/334 | 261/334 | e−136 |
| AF421352_1 | receptor 1 | | (78%), | (78%), | |
| (AF421352) | [Rattus | | Positives = | Positives = | |
| | norvegicus] | | 287/334 | 287/334 | |
| | | | (85%), | (85%), | |
| gi\|16716513\|ref\|NP_ | trace amine | 332 | 252/334 | 252/334 | e−133 |
| 444435.1\| | receptor 1 [Mus | | (75%), | (75%), | |
| (NM_053205) | musculus] | | Positives = | Positives = | |
| | | | 283/334 | 283/334 | |
| | | | (84%), | (84%), | |

Table 81F lists the domain descriptions from DOMAIN analysis results against NOV81b. This indicates that the NOV81b sequence has properties similar to those of other proteins known to contain this domain.

TABLE 81F
Domain Analysis of NOV 81b

gnl|Pfam|pfam00001, 7tm_1, 7 transmembrane receptor
(rhodopsin family).
CD-Length = 254 residues, 100.0% aligned
Score = 142 bits (358), Expect = 3e−35

The disclosed NOV81b nucleic acid of the invention encoding a GPCR-like protein includes the nucleic acid whose sequence is provided in Table 81A or a fragment thereof. The invention also includes a mutant or variant nucleic acid any of whose bases may be changed from the corresponding base shown in Table 81A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV81b protein of the invention includes the GPCR-like protein whose sequence is provided in Table 81B. The invention also includes a mutant or variant protein any of whose residues may be changed from the corresponding residue shown in Table 81B while still encoding a protein that maintains its GPCR-like activities and physiological functions, or a functional fragment thereof. In the mutant or variant protein, up to about 50 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV81b) may function as a member of a “GPCR family”. Therefore, the NOV81b nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV81b nucleic acids and proteins of the invention are useful in potential therapeutic applications implicated in diseases including but not limited to various pathologies and disorders as indicated below. For example, a cDNA encoding the GPCR-like protein (NOV81b) may be useful in gene therapy, and the GPCR-like protein (NOV81b) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV81b nucleic acid encoding the GPCR-like protein of the invention, or fragments thereof, may further be useful in diagnostic applications, wherein the presence or amount of the nucleic acid or the protein are to be assessed.

NOV81b nucleic acids and polypeptides are further useful in the generation of antibodies that bind immuno-specifically to the novel NOV81b substances for use in therapeutic or diagnostic methods. These antibodies may be generated according to methods known in the art, using prediction from hydrophobicity charts, as described in the “Anti-NOVX Antibodies” section below. The disclosed NOV81b proteins have multiple hydrophilic regions, each of which can be used as an immunogen. These novel proteins can be used in assay systems for functional analysis of various human disorders, which will help in understanding of pathology of the disease and development of new drug targets for various disorders.

NOV82

A disclosed NOV82 nucleic acid of 1033 nucleotides (also referred to as CG57845-01) encoding a GPCR-like protein is shown in Table 82A. The start and stop codons are in bold letters.

TABLE 82A
NOV82 nucleotide sequence.

(SEQ ID NO:193)

AACCATGACCAGCAATTTTTCCCAACCTGTTGTGCAGCTTTGCTATGAGGATGTGAATGGATCTTGTATT
GAAACTCCCTATTCTCCTGGGTCCCGGGTAATTCTGTACACGGCGTTTAGCTTTGGGTCTTTGCTGGCTG
TATTTGGAAATCTCTTAGTAATGACTTCTGTTCTTCATTTTAAGCAGCTGCACTCTCCAACCAATTTTCT
CATTGCCTCTCTGGCCTGTGCTGACTTCTTGGTAGGTGTGACTGTGATGCTTTTCAGCATGGTCAGGACG
GTGGAGAGCTGCTGGTATTTTGGAGCCAAATTTTGTACTCTTCACAGTTGCTGTGATGTGGCATTTTGTT
ACTCTTCTGTCCTCCACTTGTGCTTCATCTGCATCGACAGGTACATTGTGGTTACTGATCCCCTGGTCTA
TGCTACCAAGTTCACCGTGTCTGTGTCGGGAATTTGCATCAGCGTGTCCTGGATTCTGCCTCTCACGTAC
AGCGGTGCTGTGTTCTACACAGGTGTCAATGATGATGGGCTGGAGGAATTAGTAAGTGCTCTCAACTGCG
TAGGTGGCTGTCAAATTATTGTAAGTCAAGGCTGGGTGTTGATAGATTTTCTGTTATTCTTCATACCTAC
CCTTGTTATGATAATTCTTTACAGTAAGATTTTTCTTATAGCTAAACAACAAGCTATAAAAATTGAAACT
ACTAGTAGCAAAGTAGAATCATCCTCAGAGAGTTATAAAATCAGAGTGGCCAAGAGAGAGAGGAAAGCAG
CTAAAACCCTGGGGGTCACGGTACTAGCATTTGTTATTTCATGGTTACCGTATACAGTTCATATATTAAT
TGATGCCTTTATGGGCTTCCTGACCCCTGCCTATATCTATGAAATTTGCTGTTGGAGTGCTTATTATAAC
TCAGCCATGAATCCTTTGATTTATGCTCTATTTTATCCTTGGTTTAGGAAAGCCATAAAACTTATTTTAA
GTGGAGATGTTTTAAAGGCTAGTTCATCAACCATTAGTTTATTTTTAGAATAA

The disclosed NOV82 polypeptide (SEQ ID NO:194) encoded by SEQ ID NO:193 has 342 amino acid residues and is presented in Table 82B using the one-letter amino acid code.

TABLE 82B
Encoded NOV82 protein sequence.

(SEQ ID NO:194)

MTSNFSQPVVQLCYEDVNGSCIETPYSPGSRVILYTAFSFGSLLAVFGNLLVMTSVLHFKQLHSPTNFLI
ASLACADFLVGVTVMLFSMVRTVESCWYFGAKFCTLHSCCDVAFCYSSVLHLCFICIDRYIVVTDPLVYA
TKFTVSVSGICISVSWILPLTYSGAVFYTGVNDDGLEELVSALNCVGGCQIIVSQGWVLIDFLLFFIPTL
VMIILYSKIFLIAKQQAIKIETTSSKVESSSESYKIRVAKRERKAAKTLGVTVLAFVISWLPYTVDILID
AFMGFLTPAYIYEICCWSAYYNSAMNPLIYALFYPWFRKAIKLILSGDVLKASSSTISLFLE

A search of sequence databases reveals that the NOV82 amino acid sequence has 145/330 (43%) identity and 214/330 (64%) similarity with SPTREMBL-ACC:O14804 PUTATIVE NEUROTRANSMITTER RECEPTOR—Homo sapiens. Public amino acid databases include the GenBank databases, SwissProt, PDB and PIR.

The disclosed NOV82 polypeptide has homology to the amino acid sequences shown in the BLASTP data listed in Table 82C.

TABLE 82C
BLAST results for NOV82

| Gene Index/ | | Length | Identity | Positives | |
|---|---|---|---|---|---|
| Identifier | Protein/Organism | (aa) | (%) | (%) | Expect |
| gi\|16751917\|ref\|NP_ | G protein-coupled | 342 | 342/342 | 342/342 | e−166 |
| 444508.1\| | receptor 102; | | (100%) | (100%) | |
| (NM_053278) | trace amine | | | | |
| | receptor 5 [Homo | | | | |
| | sapiens] | | | | |
| gi\|14600102\|gb\|AAK71250.1\| | similar to trace | 374 | 274/344 | 308/344 | e−143 |
| AF380199_1 | amine receptor 4 | | (79%) | (88%) | |
| (AF380199) | (H. sapiens) | | | | |
| | [Homo sapiens] | | | | |
| gi\|17453968\|ref\|XP_ | Similar to trace | 345 | 273/343 | 303/343 | e−142 |
| 069046.1\| | amine receptor 4 | | (79%) | (87%) | |
| (XM_069046) | [Rattus | | | | |
| | norvegicus] | | | | |
| gi\|14600086\|gb\|AAK71242.1\| | trace amine | 345 | 266/343 | 302/343 | e−142 |
| AF380191_1 | receptor 4 | | (77%) | (87%) | |
| (AF380191) | [Rattus | | | | |
| | norvegicus] | | | | |
| gi\|14600100\|gb\|AAK71249.1\| | trace amine | 344 | 274/344 | 305/344 | e−141 |
| AF380198_1 | receptor 10 | | (79%) | (88%) | |
| (AF380198) | [Rattus | | | | |
| | norvegicus] | | | | |

Table 82D lists the domain descriptions from DOMAIN analysis results against NOV82. This indicates that the NOV82 sequence has properties similar to those of other proteins known to contain this domain.

TABLE 82D
Domain Analysis of NOV82

gnl|Pfam|pfam00001, 7tm_1, 7 transmembrane receptor
(rhodopsin family).
CD-Length = 254 residues, 100.0% aligned
Score = 132 bits (331), Expect = 4e−32

The disclosed NOV82 nucleic acid of the invention encoding a GPCR-like protein includes the nucleic acid whose sequence is provided in Table 82A or a fragment thereof. The invention also includes a mutant or variant nucleic acid any of whose bases may be changed from the corresponding base shown in Table 82A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV82 protein of the invention includes the GPCR-like protein whose sequence is provided in Table 82B. The invention also includes a mutant or variant protein any of whose residues may be changed from the corresponding residue shown in Table 82B while still encoding a protein that maintains its GPCR-like activities and physiological functions, or a functional fragment thereof. In the mutant or variant protein, up to about 57 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV82) may function as a member of a “GPCR family”. Therefore, the NOV82 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV82 nucleic acids and proteins of the invention are useful in potential therapeutic applications implicated in diseases including but not limited to various pathologies and disorders as indicated below. For example, a cDNA encoding the GPCR-like protein (NOV82) may be useful in gene therapy, and the GPCR-like protein (NOV82) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV82 nucleic acid encoding the GPCR-like protein of the invention, or fragments thereof, may further be useful in diagnostic applications, wherein the presence or amount of the nucleic acid or the protein are to be assessed.

NOV82 nucleic acids and polypeptides are further useful in the generation of antibodies that bind immuno-specifically to the novel NOV82 substances for use in therapeutic or diagnostic methods. These antibodies may be generated according to methods known in the art, using prediction from hydrophobicity charts, as described in the “Anti-NOVX Antibodies” section below. The disclosed NOV82 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV83

A disclosed NOV83 nucleic acid of 1045 nucleotides (also referred to as CG57843-01) encoding a GPCR-like protein is shown in Table 83A. The start and stop codons are in bold letters.

TABLE 83A
NOV83 nucleotide sequence.

(SEQ ID NO:195)

CGTTATGAGCAGCAATTCATCCCTGCTGGTGGCTGTGCAGCTGTGCTACGCGAACGTGAATGGGTCCTGT
GTGAAAATCCCCTTCTCGCCGGGATCCCGGGTGATTCTGTACATAGTGTTTGGCTTTGGGGCTGTGCTGG
CTGTGTTTGGAAACCTCCTGGTGATGATTTCAATCCTCCATTTCAAGCAGCTGCACTCTCCGACCAATTT
TCTCGTTGCCTCTCTGGCCTGCGCTGATTTCTTGGTGGGTGTGACTGTGATGCCCTTCAGCATGGTCAGG
ACGGTGGAGAGCTGCTGGTATTTTGGGAGGAGTTTTTGTACTTTCCACACCTGCTGTGATGTGGCATTTT
GTTACTCTTCTCTCTTTCACTTCTGCTTCATCTCCATCGACAGGTACATTGCGGTTACTGACCCCCTGGT
CTATCCTACCAAGTTCACCGTATCTGTGTCAGGAATTTGCATCAGCGTGTCCTCGATCCTGCCCCTCATG
TACAGCGGTGCTGTGTTCTACACAGGTGTCTATGACGATGGGCTGGAGGAATTATCTGATGCCCTAAACT
GTATAGGAGGTTGTCAGACCGTTGTAAATCAAAACTGGGTGTTGACAGATTTTCTATCCTTCTTTATACC
TACCTTTATTATGATAATTCTGTATGGTAACATATTTCTTGTGGCTAGACGACAGGCGAAAAAGATAGAA
AATACTGGTAGCAAGACAGAATCATCCTCAGAGAGTTACAAAGCCAGAGTGGCCAGGAGAGAGAGAAAAG
CAGCTAAAACCCTGGGGGTCACAGTGGTAGCATTTATGATTTCATGGTTACCATATAGCATTGATTCATT
AATTGATGCCTTTATGGGCTTTATAACCCCTGCCTGTATTTATGAGATTTGCTGTTGGTGTCCTTATTAT
AACTCAGCCATGAATCCTTTGATTTATGCTTTATTTTACCCATGGTTTAGGAAAGCAATAAAAGTTATTG
TAACTGGTCAGGTTTTAAAGAACAGTTCAGCAACCATGAATTTGTTTTCTGAACATATATAAGCA

The disclosed NOV83 polypeptide (SEQ ID NO:196) encoded by SEQ ID NO:195 has 345 amino acid residues and is presented in Table 83B using the one-letter amino acid code.

TABLE 83B
Encoded NOV83 protein sequence.

(SEQ ID NO:196)

MSSNSSLLVAVQLCYANVNGSCVKIPFSPGSRVILYIVFGFGAVLAVFGNLLVMISILHFKQLHSPTNFL
VASLACADFLVGVTVMPPSMVRTVESCWYFGRSFCTFHTCCDVAFCYSSLFHLCFISIDRYIAVTDPLVY
PTKFTVSVSGICISVSWILPLMYSGAVFYTGVYDDGLEELSDALNCIGGCQTVVNQNWVLTDFLSFFIPT
FIMIILYGNIFLVARRQAKKIENTGSKTESSSESYKARVARRERKAAKTLGVTVVAFMISWLPYSIDSLI
DAFMGFITPACIYEICCWCAYYNSAMNPLIYALFYPWFRKAIKVIVTGQVLKNSSATMNLFSEHI

A search of sequence databases reveals that the NOV83 amino acid sequence has 146/330 (44%) identity and 216/330 (65%) similarity with SPTREMBL-ACC:O14804 PUTATIVE NEUROTRANSMITTER RECEPTOR—Homo sapiens. Public amino acid databases include the GenBank databases, SwissProt, PDB and PIR.

The disclosed NOV83 polypeptide has homology to the amino acid sequences shown in the BLASTP data listed in Table 83C.

TABLE 83C
BLAST results for NOV83

| Gene Index/ | | Length | Identity | Positives | |
|---|---|---|---|---|---|
| Identifier | Protein/Organism | (aa) | (%) | (%) | Expect |
| gi\|17453968\|ref\|XP_ | similar to trace | 345 | 345/345 | 345/345 | e−180 |
| 069046.1\| | amine receptor 4 | | (100%) | (100%) | |
| (XM_069046) | (H. sapiens) | | | | |
| gi\|14600086\|gb\|AAK71242.1\| | trace amine | 345 | 302/345 | 321/345 | e−162 |
| AF380191_1 | receptor 4 | | (87%) | (92%) | |
| (AF380191) | [Rattus | | | | |
| | norvegicus] | | | | |
| gi\|14600102\|gb\|AAK71250.1\| | trace amine | 373 | 265/345 | 306/345 | e−141 |
| AF380199_1 | receptor 11 | | (76%) | (87%) | |
| (AF380199) | [Rattus | | | | |
| | norvegicus] | | | | |
| gi\|16751917\|ref\|NP_ | G protein-coupled | 342 | 273/343 | 303/343 | e−137 |
| 444508.1\| | receptor 102; | | (79%) | (87%) | |
| (NM_053278) | trace amine | | | | |
| | receptor 5 [Homo | | | | |
| | sapiens] | | | | |
| gi\|14600094\|gb\|AAK71246.1\| | trace amine | 344 | 258/345 | 302/345 | e−137 |
| AF380195_1 | receptor 7 | | (74%) | (86%) | |
| (AF380195) | [Rattus | | | | |
| | norvegicus] | | | | |

Table 83D lists the domain descriptions from DOMAIN analysis results against NOV83. This indicates that the NOV83 sequence has properties similar to those of other proteins known to contain this domain.

TABLE 83D
Domain Analysis of NOV83

gnl|Pfam|pfam00001, 7tm_1, 7 transmembrane receptor
(rhodopsin family).
CD-Length = 254 residues, 100.0% aligned
Score = 130 bits (326), Expect = 2e−31

The disclosed NOV83 nucleic acid of the invention encoding a GPCR-like protein includes the nucleic acid whose sequence is provided in Table 83A or a fragment thereof. The invention also includes a mutant or variant nucleic acid any of whose bases may be changed from the corresponding base shown in Table 83A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV83 protein of the invention includes the GPCR-like protein whose sequence is provided in Table 83B. The invention also includes a mutant or variant protein any of whose residues may be changed from the corresponding residue shown in Table 83B while still encoding a protein that maintains its GPCR-like activities and physiological functions, or a functional fragment thereof. In the mutant or variant protein, up to about 56 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV83) may function as a member of a “GPCR family”. Therefore, the NOV83 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV83 nucleic acids and proteins of the invention are useful in potential therapeutic applications implicated in diseases including but not limited to various pathologies and disorders as indicated below. For example, a cDNA encoding the GPCR-like protein (NOV83) may be useful in gene therapy, and the GPCR-like protein (NOV83) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV83 nucleic acid encoding the GPCR-like protein of the invention, or fragments thereof, may further be useful in diagnostic applications, wherein the presence or amount of the nucleic acid or the protein are to be assessed.

NOV83 nucleic acids and polypeptides are further useful in the generation of antibodies that bind immuno-specifically to the novel NOV83 substances for use in therapeutic or diagnostic methods. These antibodies may be generated according to methods known in the art, using prediction from hydrophobicity charts, as described in the “Anti-NOVX Antibodies” section below. The disclosed NOV83 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV84

NOV84 includes two GPCR-like proteins disclosed below. The disclosed sequences have been named NOV84a and NOV84b.

NOV84a

A disclosed NOV84a nucleic acid of 948 nucleotides (also referred to as CG57841-01) encoding a GPCR-like protein is shown in Table 84A. The start and stop codons are in bold letters.

TABLE 84A
NOV84a nucleotide sequence.

(SEQ ID NO:197)

CCTTTGTAAATGGCCTTGGGGAATCACAGCACCATCACCGAGTTCCTCCTCCTTGGGCTGTCTGCCGACC
CCAACATCCGGGCTCTGCTCTTTGTGCTGTTCCTGGGGATTTACCTCCTGACCATAATGGAAAACCTGAT
GCTGCTGCTCATGATCAGGGCTGATTCTTGTCTCCATAAGCCCATGTATTTCTTCCTGAGTCACCTCTCT
TTTGTTGATCTCTGCTTCTCTTCAGTCATTGTGCCCAAGATGCTGGAGAACCTCCTGTCACAGAGGAAAA
CCATTTCAGTAGAGGGCTGCCTGCCTCAGGTCTTCTTTGTGTTTGTCACTGCAGGGACTGAAGCCTGCCT
TCTCTCAGGGATGGCCTATGACCGCCATGCTGCCATCTGCCGCCCACTACTTTATGGACAGATCATGGGT
AAACAGCTGTATATGCACCTTGTGTGGGGCTCATGGGGACTGGGCTTTCTGGACGCACTCATCAATGTCC
TCCTAGCTGTAAACATGGTCTTTTGTGAAGCCAAAATCATTCACCACTACAGCTATGAGATGCCATCCCT
CCTCCCTCTGTCCTCCTCTGATATCTCCAGAAGCCTCATCGCCTTGCTCTGCTCCACTCTCCTACATGGG
CTGGGAAACTTCCTTTTGGTCTTCTTATCCTACACCCGTATAATCTCTACCATCCTAAGCATCAGCTCTA
CCTCGGGCAGAAGCAAGGCCTTCTCCACCTGCTCTGCCCACCTCACTGCAGTGACACTTTACTATGGCTC
AGGTTTGCTCCGCCATCTCATGCCAAACTCAGGTTCCCCCATAGAGTTGATCTTCTCTGTGCAGTATACT
GTAGTCACTCCCATGCTGAATTCCCTCATCTATAGCCTGAAAAATAAGGAAGTGAAGGTAGCTCTGAAAA
GAACTTTGGAAAAATATTTGCAATATACCAGACGTTGA

The disclosed NOV84a polypeptide (SEQ ID NO:198) encoded by SEQ ID NO:197 has 312 amino acid residues and is presented in Table 84B using the one-letter amino acid code.

TABLE 84B
Encoded NOV84a protein sequence.

(SEQ ID NO:198)

MALGNHSTITEFLLLGLSADPNIRALLFVLFLGIYLLTIMENLMLLLMIRADSCLHKPMYFFLSHLSFVD
LCFSSVIVPKMLENLLSQRKTISVEGCLAQVFFVFVTAGTEACLLSGMAYDRHAAICRPLLYGQIMGKQL
YMHLVWGSWGLGFLDALINVLLAVNMVFCEAKIIHHYSYEMPSLLPLSCSDISRSLIALLCSTLLHGLGN
FLLVFLSYTRIISTILSISSTSGRSKAFSTCSAHLTAVTLYYGSGLLRHLMPNSGSPIELIFSVQYTVVT
PMLNSLIYSLKNKEVKVALKRTLEKYLQYTRR

A search of sequence databases reveals that the NOV84a amino acid sequence has 138/309 (44%) identity and 203/309 (65%) similarity with TREMBLNEW-ACC:AAG39860 ODORANT RECEPTOR K15—Mus musculus. Public amino acid databases include the GenBank databases, SwissProt, PDB and PIR.

NOV84b

A disclosed NOV84b nucleic acid of 1039 nucleotides (also referred to as CG57841-02) encoding a GPCR-like protein is shown in Table 84C. The start and stop codons are in bold letters.

TABLE 84C
NOV84b nucleotide sequence.

| | |
|---|---|
| GAATGGATGCCCTTTTGCCACAATATAATTAATATTTCCTGTGTGAAAAACAACTGGTCAA | (SEQ ID NO:199) |
| ATGATGTCCGTGCTTCCCTGTACAGTTTAATGGTGCTCATAATTCTGACCACACTCGTTG | |
| GCAATCTGATAGTTATTGTTTCTATATCACACTTCAAACAACTTCATACCCCAACAAATT | |
| GGCTCATTCATTCCATGGCCACTGTGGACTTTCTTCTGGGGTGTCTGGTCATGCCTTACA | |
| GTATGGTGAGATCTGCTGAGCACTGTTGGTATTTTGGAGAAGTCTTCTGTAAAATTCACA | |
| CAAGCACCGACATTATGCTGAGCTCAGCCTCCATTTTCCATTTGTCTTTCATCTCCATTG | |
| ACCGCTACTATGCTGTGTGTGATCCACTGAGATATAAAGCCAAGATGAATATCTTGGTTA | |
| TTTGTGTGATGATCTTCATTAGTTGGAGTGTCCCTGCTGTTTTTGCATTTGGAATGATCT | |
| TTCTGGAGCTAAACTTCAAAGGCGCTGAAGAGATATATTACAAACATGTTCACTGCAGAG | |
| GAGGTTGCTCTGTCTTCTTTAGCAAAATATCTGGGGTACTGACCTTTATGACTTCTTTTT | |
| ATATACCTGGATCTATTATGTTATGTGTCTATTACAGAATATATCTTATCGCTAAAGAAC | |
| AGGCAAGATTAATTAGTGATGCCAATCAGAAGCTCCAAATTGGATTGGAAATGAAAAATG | |
| GAATTTCACAAAGCAAAGAAAGGAAAGCTGTGAAGACATTGGGGATTGTGATGGGAGTTT | |
| TCCTAATATGCTGGTGCCCTTTCTTTATCTGTACAGTCATGGACCCTTTTCTTCACTACA | |
| TTATTCCACCTACTTTGAATGATGTATTGATTTGGTTTGGCTACTTGAACTCTACATTTA | |
| ATCCAATGGTTTATGCATTTTTCTATCCTTGGTTTAGAAAAGCACTGAAGATGATGCTGT | |
| TTGGTAAAATTTTCCAAAAAGATTCATCCAGGTGTAAATTATTTTTGGAATTGAGTTCAT | |
| AGAATTATTATATTTTACT | |

In a search of public sequence databases, the NOV84b nucleic acid sequence, located on chromsome 6 has 616 of 979 bases (62%) identical to a gb:GENBANK-ID:HSU88828|acc:U88828.1 mRNA from Homo sapiens (Homo sapiens serotonin-4-receptor-like pseudogene). Public nucleotide databases include all GenBank databases and the GeneSeq patent database.

The disclosed NOV84b polypeptide (SEQ ID NO:200) encoded by SEQ ID NO:199 has 339 amino acid residues and is presented in Table 84D using the one-letter amino acid code. Signal P, Psort and/or Hydropathy results predict that NOV84b has a signal peptide and is likely to be localized in the plasma membrane with a certainty of 0.6000. The most likely cleavage site for a NOV84b peptide is between amino acids 47 and 48.

TABLE 84D
Encoded NOV84b protein sequence.

| | |
|---|---|
| MMPFCHNIINISCVKNNWSNDVRASLYSLMVLIILTTLVGNLIVIVSISHFKQLHTPTNW | (SEQ ID NO:200) |
| LIHSMATVDFLLGCLVMPYSMVRSAEHCWYFGEVFCKIHTSTDIMLSSASIFHLSFISID | |
| RYYAVCDPLRYKAKMNILVICVMIFISWSVPAVFAFGMIFLELNFKGAEEIYYKHVHCRG | |
| GCSVFFSKISGVLTFMTSFYIPGSIMLCVYYRIYLIAKEQARLISDANQKLQIGLEMKNG | |
| ISQSKERKAVKTLGIVMGVFLICWCPFFICTVMDPFLHYIIPPTLNDVLIWFGYLNSTFN | |
| PMVYAFFYPWFRKALKMMLFGKIFQKDFQKDSSRCKLFLELSS | |

A search of sequence databases reveals that the NOV84b amino acid sequence has 152 of 299 amino acid residues (50%) identical to, and 206 of 299 amino acid residues (68%) similar to, the 343 amino acid residue ptnr:SPTREMBL-ACC:Q9P1P4 protein from Homo sapiens (Human) (G PROTEIN-COUPLED RECEPTOR 57). Public amino acid databases include the GenBank databases, SwissProt, PDB and PIR.

NOV84b is expressed in at least Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

The disclosed NOV84b polypeptide has homology to the amino acid sequences shown in the BLASTP data listed in Table 84E.

TABLE 84E
BLAST results for NOV84b

| Gene Index/ | | Length | Identity | Positives | |
|---|---|---|---|---|---|
| Identifier | Protein/Organism | (aa) | (%) | (%) | Expect |
| gi\|17474603\|ref\|XP_ | similar to | 312 | 312/312 | 312/312 | e−145 |
| 062552.1\| | olfactory | | (100%) | (100%) | |
| (XM_062552) | receptor [Homo | | | | |
| | sapiens] | | | | |
| gi\|18479402\|gb\|AAL60715.1\| | olfactory | 309 | 216/304 | 254/304 | e−105 |
| (AY073052) | receptor MOR160-1 | | (71%) | (83%) | |
| | [Mus musculus] | | | | |
| gi\|18480580\|gb\|AAL61304.1\| | olfactory | 308 | 212/307 | 256/307 | e−95 |
| (AY073641) | receptor MOR160-2 | | (69%) | (83%) | |
| | [Mus musculus] | | | | |
| gi\|18480924\|gb\|AAL61476.1\| | olfactory | 311 | 211/309 | 258/309 | e−94 |
| (AY073813) | receptor MOR160-5 | | (68%) | (83%) | |
| | [Mus musculus] | | | | |
| gi\|18480922\|gb\|AAL61475.1\| | olfactory | 305 | 184/302 | 233/302 | 6e−84 |
| (AY073812) | receptor MOR160-4 | | (60%) | (76%) | |
| | [Mus musculus] | | | | |

Table 84F lists the domain descriptions from DOMAIN analysis results against NOV84b. This indicates that the NOV84b sequence has properties similar to those of other proteins known to contain this domain.

TABLE 84F
Domain Analysis of NOV84b

gnl|Pfam|pfam00001, 7tm_1, 7 transmembrane receptor
(rhodopsin family).
CD-Length = 254 residues, 44.9% aligned
Score = 68.6 bits (166), Expect = 5e−13

The disclosed NOV84b nucleic acid of the invention encoding a GPCR-like protein includes the nucleic acid whose sequence is provided in Table 84A or a fragment thereof. The invention also includes a mutant or variant nucleic acid any of whose bases may be changed from the corresponding base shown in Table 84A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, Up to about 38 percent of the bases may be so changed.

The disclosed NOV84b protein of the invention includes the GPCR-like protein whose sequence is provided in Table B. The invention also includes a mutant or variant protein any of whose residues may be changed from the corresponding residue shown in Table 84B while still encoding a protein that maintains its GPCR-like activities and physiological functions, or a functional fragment thereof. In the mutant or variant protein, up to about 50 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV84b) may function as a member of a “GPCR family”. Therefore, the NOV84b nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV84b nucleic acids and proteins of the invention are useful in potential therapeutic applications implicated in diseases including but not limited to various pathologies and disorders as indicated below. For example, a cDNA encoding the GPCR-like protein (NOV84b) may be useful in gene therapy, and the GPCR-like protein (NOV84b) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV84b nucleic acid encoding the GPCR-like protein of the invention, or fragments thereof, may further be useful in diagnostic applications, wherein the presence or amount of the nucleic acid or the protein are to be assessed.

NOV84b nucleic acids and polypeptides are further useful in the generation of antibodies that bind immuno-specifically to the novel NOV84b substances for use in therapeutic or diagnostic methods. These antibodies may be generated according to methods known in the art, using prediction from hydrophobicity charts, as described in the “Anti-NOVX Antibodies” section below. The disclosed NOV84b proteins have multiple hydrophilic regions, each of which can be used as an immunogen. These novel proteins can be used in assay systems for functional analysis of various human disorders, which will help in understanding of pathology of the disease and development of new drug targets for various disorders.

NOV85

A disclosed NOV85 nucleic acid of 963 nucleotides (also referred to as CG57839-01) encoding a GPCR-like protein is shown in Table 85A. The start and stop codons are in bold letters.

TABLE 85A
NOV85 nucleotide sequence.

(SEQ ID NO:201)

AACATGGAAAGCAATCAGACCTGCATCACAGAAGTCATCCTGTTGGGATTCCAGGTTGGACCAGCTCTGG
AGTTGTTCCTCTTTGGGTTTTTCTTGCTATTCTACAGCTTAACCCTGATGGGAAATTTGGACTCTAGACT
GCACACACCCATGTATGTCTTCCTGTCACATCTGGCCATTGTGGACATGTCCTATGCCTCGAGTACTGTC
CCTAAGATGCTAGCAAATCTTGTGATGCACAAAAAAGTCATCTCCTTTGCTCCTTGCATACTTCAGACTT
TTTTGTATTTGGCGTTTGCTATTACAGAGTGTCTGATTTTGGTGATGATGTGCTATGATCGGTATGTGGC
AATCTGTCACCCCTTGCAATACACCCTCATTATGAACTGGAGAGTGTGCACTGTCCTGGCCTCAACTTGC
TGGATATTTAGCTTTCTCTTGGCTCTGCTCCATATTACTCTTATTCTGAGGCTGCCTTTTTGTGGCCACA
AAACATCAACCACTTTTTTTTTGTGGCCACAAAAGATCAACCACTTTTTCTGTCAAATCATGTCCGTATT
CAAATTGGCCTGTGCTGACACTAGGCTCAACCAGGTGGTCCTATTTGCGGGTTCTGCGTTCATCTTAGTG
GGGCCGCTCTGCCTGGTGCTGGTCTCCTACTTGCACATCCTGGTGGCCATCTTGAGGATCCAGTCTGGGG
AGGGCCGCAGAAAGGCCTTCTCTACCTGCTCCTCCCACCTCTGCGTCGTGGGGCTTTTCTTTGGCAGCGC
CATTGTCATGTACATGGCCCCCAAGTCAAGCCATTCTCAAGAACGGAGGAAGATCCTTTCCCTGTTTTAC
AGCCTTTTCAACCCGATCCTGAACCCCCTCATCTACAGCCTTAATGCAGAGGTGAAAGGGGCTCTAAAGA
GAGTCCTTTGGAAACAGAGATCAATTGAAGAATCATTTGAGATTTCCTGAGAA

The disclosed NOV85 polypeptide (SEQ ID NO:202) encoded by SEQ ID NO:201 has 318 amino acid residues and is presented in Table 85B using the one-letter amino acid code

TABLE 85B
Encoded NOV85 protein sequence.

(SEQ ID NO:202)

MESNQTWITEVILLGFQVGPALELFLFGFFLLFYSLTLMGNLDSRLHTPMYVFLSHLAIVDMSYASSTVP
KMLANLVMHKKVISFAPCILQTFLYLAFAITECLILVMMCYDRYVAICHPLQYTLIMNWRVCTVLASTCW
IFSFLLALVHITLILRLPFCGHKRSTTFFLWPQKINHFFCQIMSVFKLACADTRLNQVVLFAGSAFILVG
PLCLVLVSYLHILVAILRIQSGEGRRKAFSTCSSHLCVVGLFFGSAIVMYMAPKSSHSQERRKILSLFYS
LFNPILNPLIYSLNAEVKGALKRVLWKQRSIEESFEIS

A search of sequence databases reveals that the NOV85 amino acid sequence has 181/311 (58%) identity and 232/311 (74%) similarity with SPTREMBL-ACC:O95047 WUGSC:H_DJ0988G15.2 PROTEIN—Homo sapiens. Public amino acid databases include the GenBank databases, SwissProt, PDB and P1R.

The disclosed NOV85 polypeptide has homology to the amino acid sequences shown in the BLASTP data listed in Table 85C.

TABLE 85C
BLAST results for NOV85

| Gene Index/ | | Length | Identity | Positives | |
|---|---|---|---|---|---|
| Identifier | Protein/Organism | (aa) | (%) | (%) | Expect |
| gi\|18565914\|ref\|XP_ | protein XP_095005 | 310 | 299/320 | 300/320 | e−134 |
| 095005.1\| | [Homo sapiens] | | (93%) | (93%) | |
| (XM_095005) | | | | | |
| gi\|18480894\|gb\|AAL61461.1\| | olfactory | 308 | 254/317 | 277/317 | e−115 |
| (AY073798) | receptor MOR261- | | (80%) | (87%) | |
| | 12 [Mus musculus] | | | | |
| gi\|18480182\|gb\|AAL61105.1\| | olfactory | 310 | 238/320 | 266/320 | e−111 |
| (AY073442) | receptor MOR261-4 | | (74%) | (82%) | |
| | [Mus musculus] | | | | |
| gi\|18480180\|gb\|AAL61104.1\| | olfactory | 310 | 235/320 | 263/320 | e−110 |
| (AY073441) | receptor MOR261-3 | | (73%) | (81%) | |
| | [Mus musculus] | | | | |
| gi\|18565912\|ref\|XP_ | similar to | 311 | 231/320 | 258/320 | e−108 |
| 069619.2\| | olfactory | | (72%) | (80%) | |
| (XM_069619 | receptor [Homo | | | | |
| | sapiens] | | | | |

Table 85D lists the domain descriptions from DOMAIN analysis results against NOV85. This indicates that the NOV85 sequence has properties similar to those of other proteins known to contain this domain.

TABLE 85D
Domain Analysis of NOV85

gnl|Pfam|pfam00001, 7tm_1, 7 transmembrane receptor
(rhodopsin family).
CD-Length = 254 residues, 94.9% aligned
Score = 111 bits (278), Expect = 5e−26

The disclosed NOV85 nucleic acid of the invention encoding a GPCR-like protein includes the nucleic acid whose sequence is provided in Table 85A or a fragment thereof. The invention also includes a mutant or variant nucleic acid any of whose bases may be changed from the corresponding base shown in Table 85A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV85 protein of the invention includes the GPCR-like protein whose sequence is provided in Table 85B. The invention also includes a mutant or variant protein any of whose residues may be changed from the corresponding residue shown in Table 85B while still encoding a protein that maintains its GPCR-like activities and physiological functions, or a functional fragment thereof. In the mutant or variant protein, up to about 42 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV85) may function as a member of a “GPCR family”. Therefore, the NOV85 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV85 nucleic acids and proteins of the invention are useful in potential therapeutic applications implicated in diseases including but not limited to various pathologies and disorders as indicated below. For example, a cDNA encoding the GPCR-like protein (NOV85) may be useful in gene therapy, and the GPCR-like protein (NOV85) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV85 nucleic acid encoding the GPCR-like protein of the invention, or fragments thereof, may further be useful in diagnostic applications, wherein the presence or amount of the nucleic acid or the protein are to be assessed.

NOV85 nucleic acids and polypeptides are further useful in the generation of antibodies that bind immuno-specifically to the novel NOV85 substances for use in therapeutic or diagnostic methods. These antibodies may be generated according to methods known in the art, using prediction from hydrophobicity charts, as described in the “Anti-NOVX Antibodies” section below. The disclosed NOV85 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV86

A disclosed NOV86 nucleic acid of 971 nucleotides (also referred to as CG-01) encoding a-like protein is shown in Table 86A. The start and stop codons are in bold letters.

TABLE 86A
NOV86 nucleotide sequence.

(SEQ ID NO:203)

TACCCTTGTTAATGGCCTTTGGGGAATTCCCCAGCCCCATTCCCCCGAGTTCCCTCCTCCCTTTGGGCTT
GTTCTGCGGACCCCCCAAACAATCCCGGGGCTTTCTGCCCTTCTTTGTGCTGTTCCTGGGGATTTACCTC
CTGACCATAATGGAAAACCTGATGCTGCTGCTCATGATCAGGGCTGATTCTTGTCTCCATAAGCCCATGT
ATTTCTTCCTGAGTCACCTCTCTTTTGTTGATCTCTGCTTCTCTTCAGTCATTGTGCCCAAGATGCTGGA
GAACCTCCTGTCACAGAGGAAAACCATTTCAGTAGAGGGCTGCCTGGCTCAGGTCTTCTTTGTGTTTGTC
ACTGCAGGGACTGAAGCCTGCCTTCTCTCAGGGATGGCCTATGACCGCCATGCTGCCATCTGCCGCCCAC
TACTTTATGGACAGATCATGGGTAAACAGCTGTATATGCACCTTGTGTGGGGCTCATGGGGACTGGGCTT
TCTGGACGCACTCATCAATGTCCTCCTAGCTGTAAACATGGTCTTTTGTGAAGCCAAAATCATTCACCAC
TACAGCTATGAGATGCCATCCCTCCTCCCTCTGTCCTGCTCTGATATCTCCAGAAGCCTCATCGCCTTGC
TCTGCTCCACTCTCCTACATGGGCTGGGAAACTTCCTTTTGGTCTTCTTATCCTACACCCGTATAATCTC
TACCATCCTAAGCATCAGCTCTACCTCGGGCAGAAGCAAGGCCTTCTCCACCTGCTCTGCCCACCTCACT
GCAGTGACACTTTACTATGGCTCAGGTTTGCTCCGCCATCTCATGCCAAACTCAGGTTCCCCCATAGAGT
TGATCTTCTCTGTGCAGTATACTGTAGTCACTCCCATGCTGAATTCCCTCATCTATAGCCTGAAAAATAA
GGAAGTGAAGGTAGCTCTGAAAAGAACTTTGGAAAAATATTTGCAATATACCAGACGTTGA

The disclosed NOV86 polypeptide (SEQ ID NO:204) encoded by SEQ ID NO:203 has 319 amino acid residues and is presented in Table 86B using the one-letter amino acid code.

TABLE 86B
Encoded NOV86 protein sequence.

(SEQ ID NO:204)

MAFGEPPSPIPPSSLLPLGLFCGPPKQSRGFLPFFVLFLGIYLLTIMENLMLLLMIRADSCLHKPMYFFL
SHLSFVDLCFSSVIVPKMLENLLSQRKTISVEGCLAQVFFVFVTAGTEACLLSGMAYDRHAAICRPLLYG
QIMGKQLYMHLVWGSWGLGFLDALINVLLAVNMVFCEAKIIHHYSYEMPSLLPLSCSDISRSLIALLCST
LLHGLGNFLLVFLSYTRIISTILSISSTSGRSKAFSTCSAHLTAVTLYYGSGLLRHLMPNSGSPIELIFS
VQYTVVTPMLNSLIYSLKNKEVKVALKRTLEKYLQYTRR

A search of sequence databases reveals that the NOV86 amino acid sequence has 134/300 (44%) identity and 192/300 (64%) similarity with SPTREMBL-ACC:O35184 OLFACTORY RECEPTOR—Rattus norvegicus. Public amino acid databases include the GenBank databases, SwissProt, PDB and PIR.

The disclosed NOV86 polypeptide has homology to the amino acid sequences shown in the BLASTP data listed in Table 86C.

TABLE 86C
BLAST results for NOV86

| Gene Index/ | | Length | Identity | Positives | |
|---|---|---|---|---|---|
| Identifier | Protein/Organism | (aa) | (%) | (%) | Expect |
| gi\|17474603\|ref\|XP_ | similar to | 312 | 290/307 | 293/307 | e−134 |
| 062552.1\| | olfactory | | (94%) | (94%), | |
| (XM_062552) | receptor [Homo | | | | |
| | sapiens] | | | | |
| gi\|18479402\|gb\|AAL60715.1\| | olfactory | 309 | 200/281 | 234/281 | 3e−97 |
| (AY073052) | receptor MOR160-1 | | (71%) | (83%) | |
| | [Mus musculus] | | | | |
| gi\|18480580\|gb\|AAL61304.1\| | olfactory | 308 | 193/280 | 236/280 | 8e−88 |
| (AY073641) | receptor MOR160-2 | | (68%) | (83%) | |
| | [Mus musculus] | | | | |
| gi\|18480924\|gb\|AAL61476.1\| | olfactory | 311 | 192/282 | 237/282 | 5e−87 |
| (AY073813) | receptor MOR160-5 | | (68%) | (83%) | |
| | [Mus musculus] | | | | |
| gi\|18480922\|gb\|AAL61475.1\| | olfactory | 305 | 170/279 | 215/279 | e−78 |
| (AY073812) | receptor MOR160-4 | | (60%) | (76%) | |
| | [Mus musculus] | | | | |

Table 86D lists the domain descriptions from DOMAIN analysis results against NOV86. This indicates that the NOV86 sequence has properties similar to those of other proteins known to contain this domain.

TABLE 86D
Domain Analysis of NOV86

gnl|Pfam|pfam00001, 7tm_1, 7 transmembrane receptor
(rhodopsin family).
CD-Length = 254 residues, 44.9% aligned
Score = 68.2 bits (165), Expect = 7e−13

The disclosed NOV86 nucleic acid of the invention encoding a GPCR-like protein includes the nucleic acid whose sequence is provided in Table 86A or a fragment thereof. The invention also includes a mutant or variant nucleic acid any of whose bases may be changed from the corresponding base shown in Table 86A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV86 protein of the invention includes the GPCR-like protein whose sequence is provided in Table 86B. The invention also includes a mutant or variant protein any of whose residues may be changed from the corresponding residue shown in Table 86B while still encoding a protein that maintains its GPCR-like activities and physiological functions, or a functional fragment thereof. In the mutant or variant protein, up to about 56 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV86) may function as a member of a “GPCR family”. Therefore, the NOV86 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV86 nucleic acids and proteins of the invention are useful in potential therapeutic applications implicated in diseases including but not limited to various pathologies and disorders as indicated below. For example, a cDNA encoding the GPCR-like protein (NOV86) may be useful in gene therapy, and the GPCR-like protein (NOV86) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV86 nucleic acid encoding the GPCR-like protein of the invention, or fragments thereof, may further be useful in diagnostic applications, wherein the presence or amount of the nucleic acid or the protein are to be assessed.

NOV86 nucleic acids and polypeptides are further useful in the generation of antibodies that bind immuno-specifically to the novel NOV86 substances for use in therapeutic or diagnostic methods. These antibodies may be generated according to methods known in the art, using prediction from hydrophobicity charts, as described in the “Anti-NOVX Antibodies” section below. The disclosed NOV86 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV87

A disclosed NOV87 nucleic acid of 1067 nucleotides (also referred to as CG56763-01) encoding a GPCR-like protein is shown in Table 87A. The start and stop codons are in bold letters.

TABLE 87A
NOV87 nucleotide sequence.

(SEQ ID NO:205)

CCCTTTCCTCTTGCTCTTTGATGTTTTGTAGGCCTGCAGCTCCCAAGCACAGAGGCATGAGTGGGGAGAA
TGTCACCAGGGTCGGCACCTTCATCCTGGTGGGCTTCCCCACGGCCCCAGGGCTGCAGTACCTGCTCTTC
CTCCTCTTCCTGCTCACCTACCTCTTTGTCCTGGTGGAGAACCTGGCCATCATCCTCACCGTCTGGAGCA
GCACCTCCCTCCACAGGCCCATGTACTACTTTCTGAGCTCCATGTCTTTCCTAGAGATCTGGTACGTGTC
TGACATCACCCCCAAGATGCTGGAGGGCTTCCTCCTCCAGCAGAAACGCATCTCTTTCGTCGGGTGCATG
ACGCAGCTCTACTTCTTCAGCTCCCTGGTGTGCACCGAGTGTGTGCTTCTGGCCTCCATGGCCTACGACC
GCTACGTGGCCATCTGCCACCCGCTGCGCTACCACGTCCTTGTGACCCCGGGGCTGTGCCTCCAGCTGGT
GGGCTTCTCCTTTGTGAGTGGCTTCACCATCTCCATGATCAAGGTCTGTTTTATCTCCACCGTCACGTTC
TGTGGCTCCAACGTCTTGAACCACTTCTTCTGTGACATTTCCCCCATCCTCAAGCTGGCCTGCACGGACT
TCTCCACTGCAGAGCTGGTGGATTTCATTCTGGCCTTCATCATCCTGGTGTTTCCACTCCTGGCCACCAT
GCTGTCATATGCGCACATCACCCTGGCTGTCCTGCGCATCCCCTCGGCCACCGGCTGCTGGAGAGCCTTC
TTCACCTGCGCCTCTCACCTCACCGTGGTCACCGTCTTCTATACAGCCTTGCTTTTCATGTATGTCCGGC
CCCAGGCCATTGATTCCCGGAGCTCCAACAAGCTCATCTCTGTTTTGTACACAGTTATCACCCCCATCTT
GAACCCCTTGATATACTGCCTGAGGAATAAGGAATTTAAGAATGCCTTGAAAAAAGCCTTCGGCTTGACG
AGCTGCGCCGTAGAGGGGAGGCTTTCTAGTCTTCTGGAACTTCATCTCCAAATACACAGCCAGCCTCTCT
GAGGAGGCCATTTGACT

The disclosed NOV87 polypeptide (SEQ ID NO:206) encoded by SEQ ID NO:205 has 343 amino acid residues and is presented in Table 87B using the one-letter amino acid code.

TABLE 87B
Encoded NOV87 protein sequence.

(SEQ ID NO:206)

MFCRPAAPKHRGMSGENVTRVGTFILVGFPTAPGLQYLLFLLFLLTYLFVLVENLAIILTVWSSTSLHRP
MYYFLSSMSFLEIWYVSDITPKMLEGFLLQQKRISFVGCMTQLYFFSSLVCTECVLLASMAYDRYVAICH
PLRYHVLVTPGLCLQLVGFSFVSGFTISMIKVCFISSVTFCGSNVLNHFFCDISPILKLACTDFSTAELV
DFILAFIILVFPLLATMLSYAHITLAVLRIPSATGCWRAFFTCASHLTVVTVFYTALLFMYVRPQAIDSR
SSNKLISVLYTVITPILNPLIYCLRNKEFKNALKKAFGLTSCAVEGRLSSLLELHLQIHSQPL

A search of sequence databases reveals that the NOV87 amino acid sequence has 211/306 (68%) identity and 250/306 (81%) similarity with TREMBLNEW-ACC:AAG45189 M51 OLFACTORY RECEPTOR—Mus musculus. Public amino acid databases include the GenBank databases, SwissProt, PDB and PIR.

The disclosed NOV87 polypeptide has homology to the amino acid sequences shown in the BLASTP data listed in Table 87C.

TABLE 87C
BLAST results for NOV87

| Gene Index/ | | Length | Identity | Positives | |
|---|---|---|---|---|---|
| Identifier | Protein/Organism | (aa) | (%) | (%) | Expect |
| gi\|17435880\|ref\|XP_ | similar to | 331 | 331/331 | 331/331 | e−150 |
| 065375.1\| | olfactory | | (100%) | (100%) | |
| (XM_065375) | receptor 41 [Homo | | | | |
| | sapiens] | | | | |
| gi\|17435888\|ref\|XP_ | similar to | 312 | 293/307 | 300/307 | e−133 |
| 065377.1\| | olfactory | | (95%) | (97%) | |
| (XM_065377) | receptor 41 [Homo | | | | |
| | sapiens] | | | | |
| gi\|18479396\|gb\|AAL60712.1\| | olfactory | 312 | 270/307 | 287/307 | e−122 |
| (AY073049) | receptor MOR103-2 | | (87%) | (92%) | |
| | [Mus musculus] | | | | |
| gi\|18479398\|gb\|AAL60713.1\| | olfactory | 312 | 261/307 | 284/307 | e−120 |
| (AY073050) | receptor MOR103-3 | | (85%) | (92%) | |
| | [Mus musculus] | | | | |
| gi\|12007416\|gb\|AAG45189.1\| | m51 olfactory | 314 | 211/306 | 250/306 | e−101 |
| (AF321234) | receptor [Mus | | (68%) | (80%) | |
| | musculus] | | | | |

Table 87D lists the domain descriptions from DOMAIN analysis results against NOV87. This indicates that the NOV87 sequence has properties similar to those of other proteins known to contain this domain.

TABLE 87D
Domain Analysis of NOV87

gnl|Pfam|pfam00001, 7tm_1, 7 transmembrane receptor
(rhodopsin family).
CD-Length = 254 residues, 100.0% aligned
Score = 98.2 bits (243), Expect = 7e−22

The disclosed NOV87 nucleic acid of the invention encoding a GPCR-like protein includes the nucleic acid whose sequence is provided in Table 87A or a fragment thereof. The invention also includes a mutant or variant nucleic acid any of whose bases may be changed from the corresponding base shown in Table 87A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV87 protein of the invention includes the GPCR-like protein whose sequence is provided in Table 87B. The invention also includes a mutant or variant protein any of whose residues may be changed from the corresponding residue shown in Table 87B while still encoding a protein that maintains its GPCR-like activities and physiological functions, or a functional fragment thereof. In the mutant or variant protein, up to about 32 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV87) may function as a member of a “GPCR family”. Therefore, the NOV87 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV87 nucleic acids and proteins of the invention are useful in potential therapeutic applications implicated in diseases including but not limited to various pathologies and disorders as indicated below. For example, a cDNA encoding the GPCR-like protein (NOV87) may be useful in gene therapy, and the GPCR-like protein (NOV87) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV87 nucleic acid encoding the GPCR-like protein of the invention, or fragments thereof, may further be useful in diagnostic applications, wherein the presence or amount of the nucleic acid or the protein are to be assessed.

NOV87 nucleic acids and polypeptides are further useful in the generation of antibodies that bind immuno-specifically to the novel NOV87 substances for use in therapeutic or diagnostic methods. These antibodies may be generated according to methods known in the art, using prediction from hydrophobicity charts, as described in the “Anti-NOVX Antibodies” section below. The disclosed NOV87 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV88

A disclosed NOV88 nucleic acid of 939 nucleotides (also referred to as CG56753-01) encoding a GPCR-like protein is shown in Table 88A. The start and stop codons are in bold letters.

TABLE 88A
NOV88 nucleotide sequence.

(SEQ ID NO:207)

ATGTCAGGAGAAAATAATTCCTCAGTGACTGAGTTCATTCTGGCTGGGCTCTCAGAACAGCCAGAGCTCC
AGCTGCCCCTCTTCCTCCTGTTCTTAGGAATCTATGTGGTCACAGTGGTGGGCAACCTGGGCATGACCAC
ACTGATTTGGCTCAGTTCTCACCTGCACACCCCTATGTACTATTTCCTCAGCAGTCTGTCCTTCATTGAC
TTCTGCCATTCCACTGTCATTACCCCTAAGATGCTGGTGAACTTTGTGACAGAGAAGAACATCATCTCCT
ACCCTGAATGCATGACTCAGCTCTACTTCTTCCTCGTTTTTGCTATTGCAGAGTCTCACATGTTGGCTGC
AATGGCGTATGACCGTTACATGGCCATCTGTAGCCCCTTGCTGTACAGTGTCATCATATCCAATAAGGCT
TGCTTTTCTCTGATTTTAGGGGTGTATATAATAGGCCTGGTTTGTGCATCAGTTCATACAGGCTGTATGT
TTAGGGTTCAATTCTGCAAATTTGATTTGATTAACCATTATTTCTGTGATCTTCTTCCCCTCCTAAAGCT
CTCTTGCTCTAGTATCTATGTCAACAAACTACTTATTCTATGTGTTGGTGCATTTAACATCCTTGTCCCC
AGCCTGACCATCCTTTGCTCTTACATCTTTATTATTGCCAGCATCCTCCACATTCGCTCCACTGAGGGCA
GGTCCAAAGCCTTCAGCACTTGTAGCTCCCACATGTTGGCGGTTGTAATCTTTTTTGGATCTGCAGCATT
CATGTACTTGCAGCCATCTTCAATCAGCTCCATGGACCAGGGGAAAGTATCCTCTGTGTTTTATACTATT
ATTGTGCCCATGTTGAACCCTCTGATTTATAGCCTGAGGAATAAAGATGTCCATGTTTCCCTGAAGAAAA
TGCTACAGAGAAGAACATTATTGTAAACA

The disclosed NOV88 polypeptide (SEQ ID NO:208) encoded by SEQ ID NO:207 has 311 amino acid residues and is presented in Table 88B using the one-letter amino acid code.

TABLE 88B
Encoded NOV88 protein sequence.

(SEQ ID NO:208)

MSGENNSSVTEFILAGLSEQPELQLPLFLLFLGIYVVTVVGNLGMTTLIWLSSHLHTPMYYFLSSLSFID
FCHSTVITPKMLVNFVTEKNIISYPECMTQLYFFLVFAIAECHMLAAMAYDRYMAICSPLLYSVIISNKA
CFSLILGVYIIGLVCASVHTGCMFRVQFCKFDLINHYFCDLLPLLKLSCSSIYVNKLLILCVGAFNILVP
SLTILCSYIFIIASILHIRSTEGRSKAFSTCSSHMLAVVIFFGSAAFMYLQPSSISSMDQGKVSSVFYTI
IVPMLNPLIYSLRNKDVHVSLKKMLQRRTLL

A search of sequence databases reveals that the NOV88 amino acid sequence has 239/311 (76%) identity and 275/311 (88%) similarity with TREMBLNEW-ACC:AAG39856 ODORANT RECEPTOR K11—Mus musculus. Public amino acid databases include the GenBank databases, SwissProt, PDB and PIR.

The disclosed NOV88 polypeptide has homology to the amino acid sequences shown in the BLASTP data listed in Table 88C.

TABLE 88C
BLAST results for NOV88

| Gene Index/ | | Length | Identity | Positives | |
|---|---|---|---|---|---|
| Identifier | Protein/Organism | (aa) | (%) | (%) | Expect |
| gi\|18578547\|ref\|XP_ | olfactory | 311 | 311/311 | 311/311 | e−141 |
| 090109.1\| | receptor, family | | (100%) | (100%) | |
| (XM_090109) | 8, subfamily G, | | | | |
| | member 1 [Homo | | | | |
| | sapiens] | | | | |
| gi\|17472672\|ref\|XP_ | similar to | 311 | 263/311 | 285/311 | e−122 |
| 061794.1\| | odorant receptor | | (84%) | (91%) | |
| (XM_061794) | K11 [Homo | | | | |
| | sapiens] | | | | |
| gi\|18479824\|gb\|AAL60926.1\| | olfactory | 314 | 240/311 | 276/311 | e−114 |
| (AY073263) | receptor MOR171- | | (77%) | (88%) | |
| | 5 [Mus musculus] | | | | |
| gi\|11692519\|gb\|AAG39856.1\| | odorant receptor | 314 | 239/311 | 275/311 | e−113 |
| AF282271_1 | K11 [Mus | | (76%) | (87%) | |
| (AF282271) | musculus] | | | | |
| gi\|17472670\|ref\|XP_ | similar to | 258 | 258/258 | 258/258 | e−113 |
| 061793.1\| | odorant receptor | | (100%) | (100%) | |
| (XM_061793) | K15 [Homo | | | | |
| | sapiens] | | | | |

Table 88D lists the domain descriptions from DOMAIN analysis results against NOV88. This indicates that the NOV88 sequence has properties similar to those of other proteins known to contain this domain.

TABLE 88D
Domain Analysis of NOV88

gnl|Pfam|pfam00001, 7tm_1, 7 transmembrane receptor
(rhodopsin family).
CD-Length = 254 residues, 100.0% aligned
Score = 88.6 bits (218), Expect = 5e−19

The disclosed NOV88 nucleic acid of the invention encoding a GPCR-like protein includes the nucleic acid whose sequence is provided in Table 88A or a fragment thereof. The invention also includes a mutant or variant nucleic acid any of whose bases may be changed from the corresponding base shown in Table 88A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, Up to about 5 percent of the bases may be so changed.

The disclosed NOV88 protein of the invention includes the GPCR-like protein whose sequence is provided in Table 88B. The invention also includes a mutant or variant protein any of whose residues may be changed from the corresponding residue shown in Table 88B while still encoding a protein that maintains its GPCR-like activities and physiological functions, or a functional fragment thereof. In the mutant or variant protein, up to about 24 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV88) may function as a member of a “GPCR family”. Therefore, the NOV88 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV88 nucleic acids and proteins of the invention are useful in potential therapeutic applications implicated in diseases including but not limited to various pathologies and disorders as indicated below. For example, a cDNA encoding the GPCR-like protein (NOV88) may be useful in gene therapy, and the GPCR-like protein (NOV88) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV88 nucleic acid encoding the GPCR-like protein of the invention, or fragments thereof, may further be useful in diagnostic applications, wherein the presence or amount of the nucleic acid or the protein are to be assessed.

NOV88 nucleic acids and polypeptides are further useful in the generation of antibodies that bind immuno-specifically to the novel NOV88 substances for use in therapeutic or diagnostic methods. These antibodies may be generated according to methods known in the art, using prediction from hydrophobicity charts, as described in the “Anti-NOVX Antibodies” section below. The disclosed NOV88 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV89

A disclosed NOV89 nucleic acid of 1003 nucleotides (also referred to as CG57670-01) encoding a GPCR-like protein is shown in Table 89A. The start and stop codons are in bold letters.

TABLE 89A
NOV89 nucleotide sequence.

| | |
|---|---|
| TGTTCCATACATTATTTTGTCTTTTGTCTGAAGCAATGCTGAATACAACCTCAGTCACTGAATT | (SEQ ID NO:209) |
| TCTCCTTTTGGGAGTGACAGACATTCAAGAACTGCAGCCTTTTCTCTTCGTTGTTTTCCTTACC | |
| ATCTACTTCATCAGTGTGGCTGGGAATGGAGCCATTCTGATGATTGTCATCTCTGATCCTAGAC | |
| TCCATTCCCCTATGTATTTCTTCCTGGGAAACCTGTCCTGCCTGGACATCTGCTACTCCAGCGT | |
| AACACTGCCAAAAATGCTGCAGAACTTCCTCTCTGCACACAAAGCAATTTCTTTCTTGGGATGC | |
| ATAAGCCAACTCCATTTCTTCCACTTCCTGGGCAGCACAGAGGCCATGTTGTTGGCCGTGATGG | |
| CATTTGACCGCTTTGTGGCTATTTGCAAGCCACTTCGCTACACTGTCATTATGAACCCTCAGCT | |
| CTGTACCCAGATGGCCATCACAATCTGGATGATTGGTTTTTTCCATGCCCTGCTGCACTCCCTA | |
| ATGACCTCTCGCTTGAACTTCTGTGGTTCTAACCGTATCTATCACTTCTTCTGTGATGTGAAGC | |
| CATTGCTAAAGCTGAGCCTTAATCAGTGGCTGCTCAGTACTGTCACAGGGACAATCGCCATGGG | |
| CCCCTTCTTTCTCACATTACTCTCCTATTTCTACATTATCACCCATCTCTTCTTCAAGACTCAT | |
| TCTTTTAGCATGCTCCGCAAAGCACTGTCCACTTGTGCCTCCCACTTCATGGTAGTTATTCTTT | |
| TGTATGCACCTGTTCTCTTCACCTATATTCATCATGCCTCAGGGACCTCCATGGACCAGGACCG | |
| GATCACTGCCATCATGTATACTGTGGTCACTCCAGTACTAAACCCACTGATCTACACTTTGAGG | |
| AACAAGGAAGTGAAAGGGGCCTTTAATAGAGCAATGAAAAGGTGGCTTTGGCCTAAAGAAATCT | |
| TGAAGAACTCTTCTGAAGCATAAATAAACAATTAAAAAGATGA | |

The disclosed NOV89 polypeptide (SEQ ID NO:210) encoded by SEQ ID NO:209 has 315 amino acid residues and is presented in Table 89B using the one-letter amino acid code

TABLE 89B
Encoded NOV89 protein sequence.

(SEQ ID NO:219)

MLNTTSVTEFLLLGVTDIQELQPFLFVVFLTIYFISVAGNGAILMIVISDPRLHSPMYFFLGNLSCLDIC
YSSVTLPKMLQNFLSAHKAISFLGCISQLHFFHFLGSTEAMLLAVMAFDRFVAICKPLRYTVIMNPQLCT
QMAITIWMIGFFHALLHSLMTSRLNFCGSNRIYHFFCDVKPLLKLSLNQWLLSTVTGTIAMGPFFLTLLS
YFYIITHLFFKTHSFSMLRKALSTCASHFMVVILLYAPVLFTYIHHASGTSMDQDRITAIMYTVVTPVLN
PLIYTLRNKEVKGAFNRAMKRWLWPKEILKNSSEA

A search of sequence databases reveals that the NOV89 amino acid sequence has similarity with SPTREMBL-ACC:Q9UGF7 BA150A6.1 (NOVEL 7 TRANSMEMBRANE RECEPTOR(RHODOPSIN FAMILY)(OLFACTORY RECEPTOR LIKE) PROTEIN (HS6M1-27))—Homo sapiens. Public amino acid databases include the GenBank databases, SwissProt, PDB and PIR.

The disclosed NOV89 polypeptide has homology to the amino acid sequences shown in the BLASTP data listed in Table 89C.

TABLE 89C
BLAST results for NOV89

| Gene Index/ | | Length | Identity | Positives | |
|---|---|---|---|---|---|
| Identifier | Protein/Organism | (aa) | (%) | (%) | Expect |
| gi\|17464355\|ref\|XP_ | similar to | 284 | 279/315 | 280/315 | e−130 |
| 069464.1\| | olfactory | | (88%) | (88%) | |
| (XM_069464) | receptor, family | | | | |
| | 12, subfamily D, | | | | |
| | member 2 [Homo | | | | |
| | sapiens] | | | | |
| gi\|18563691\|ref\|XP_ | hypothetical | 284 | 278/315 | 279/315 | e−130 |
| 094753.1\| | protein XP_094753 | | (88%) | (88%) | |
| (XM_094753) | [Homo sapiens] | | | | |
| gi\|7363443\|ref\|NP_039224.1\| | olfactory | 307 | 269/306 | 281/306 | e−126 |
| (NM_013936) | receptor, family | | (87%) | (90%) | |
| | 12, subfamily D, | | | | |
| | member 2 [Homo | | | | |
| | sapiens] | | | | |
| gi\|18563689\|ref\|XP_ | similar to | 307 | 268/306 | 280/306 | e−125 |
| 084201.1\| | olfactory | | (87%) | (90%) | |
| (XM_084201) | receptor, family | | | | |
| | 12, subfamily D, | | | | |
| | member 2 (H. | | | | |
| | sapiens) [Homo | | | | |
| | sapiens] | | | | |
| gi\|15020328\|emb\|CAC44545.1\| | bM332P19.2 (novel | 308 | 241/308 | 269/308 | e−117 |
| (AL133159) | 7 transmembrane | | (78%) | (87%) | |
| | receptor | | | | |
| | (rhodopsin | | | | |
| | family) | | | | |
| | (olfactory | | | | |
| | receptor like) | | | | |
| | protein (mm17M1- | | | | |
| | 13); ortholog of | | | | |
| | human DJ994E9.8 | | | | |
| | (HS6M1-20)) [Mus | | | | |
| | musculus] | | | | |

Table 89D lists the domain descriptions from DOMAIN analysis results against NOV89. This indicates that the NOV89 sequence has properties similar to those of other proteins known to contain this domain.

TABLE 89D
Domain Analysis ofNOV89

gnl|Pfam|pfam00001, 7tm_1, 7 transmembrane receptor
(rhodopsin family).
CD-Length = 254 residues, 100.0% aligned
Score = 101 bits (251), Expect = 7e−23

The disclosed NOV89 nucleic acid of the invention encoding a GPCR-like protein includes the nucleic acid whose sequence is provided in Table 89A or a fragment thereof. The invention also includes a mutant or variant nucleic acid any of whose bases may be changed from the corresponding base shown in Table 89A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids wh ose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV89 protein of the invention includes the GPCR-like protein whose sequence is provided in Table 89B. The invention also includes a mutant or variant protein any of whose residues may be changed from the corresponding residue shown in Table 89B while still encoding a protein that maintains its GPCR-like activities and physiological functions, or a functional fragment thereof. In the mutant or variant protein, up to about 5 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV89) may function as a member of a “GPCR family”. Therefore, the NOV89 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV89 nucleic acids and proteins of the invention are useful in potential therapeutic applications implicated in diseases including but not limited to various pathologies and disorders as indicated below. For example, a cDNA encoding the GPCR-like protein (NOV89) may be useful in gene therapy, and the GPCR-like protein (NOV89) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV89 nucleic acid encoding the GPCR-like protein of the invention, or fragments thereof, may further be useful in diagnostic applications, wherein the presence or amount of the nucleic acid or the protein are to be assessed.

NOV89 nucleic acids and polypeptides are further useful in the generation of antibodies that bind immuno-specifically to the novel NOV89 substances for use in therapeutic or diagnostic methods. These antibodies may be generated according to methods known in the art, using prediction from hydrophobicity charts, as described in the “Anti-NOVX Antibodies” section below. The disclosed NOV89 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV90

A disclosed NOV90 nucleic acid of 950 nucleotides (also referred to as CG57676-01) encoding a GPCR-like protein is shown in Table 90A. The start and stop codons are in bold letters.

TABLE 90A
NOV90 nucleotide sequence.

(SEQ ID NO:211)

GTAATAGGAAATGAATGATGATGGAAAAGTCAATGCTAGCTCTGAGGGGTACTTT
ATTTTAGTTGGATTTTCTAATTGGCCTTATCTGGAAGTAGTTCTCTTTGTGGTTATTTTGATCTTCTGCT
TGATGACACTGATAGGAAACCTGTTCATCATCATCCTGACGTACCTGGACTCCCATCTCCATACTCCCTT
GTATTTCTTCCTTTCAAATCTCTCATTTCTGGATCTCTGCTACACCACCAGCTCTATCCCTCAGTTGCTG
GTCAGTCTCTGGGGTGTGGAAAAGACCATTTCTTATGCTGGTTGCATGGTTCAACTTTACTTTTTTCTCA
CACTGGGAACCACAGAGTGTGTCCTACTGGTGGTGATGTCCTATGACCGTTATGCAGCTGTGTGTAGACC
TTTGCATTACACTGTCCTCATGCACTCTCGTTTCTGCCACTTGTTGGCTGTGGCTTCTTGGGTAAGTGGT
TTTACAAACCCAGCACTTCATTCCTCCTTCACCTTCTGGGTACCTCTGTGTGGACACCGCCAAATAGATC
ACTTTTTCTGTGAAGTTCCGGCACTTTTAAGATTATCATTTGTCAATACCCGTGAAAATAAACTGACCCT
CATGATCACAAGCTCCATTTTTGTTCTGCTACTTCTCACCCTCATTTTCACTTCCTATGGTGCTATTGCC
CAGGCTGTACTGAGGATGCAGTCAACCACTGGGCTTCAGAAAGTATTTGGAACATGTGGAGCTCATCATA
TGGTTGTATCTCTCTTTTTCATTCCGGCCATGTGCATGTATCTCCAGCCACCATCAGGGAATTCTCAAGA
TCAAGGCAAGTTCATTGCTCTCTTTTATACTGTTGTTACACCTAGTCTTAACCCTCTAATCTACACCCTC
AGAAACAAAGATGTAAGAGGGGTAGTGAAGAGACTAAGGGGGTGGGAGTGAGCCT

The disclosed NOV90 polypeptide (SEQ ID NO:212) encoded by SEQ ID NO:211 has 311 amino acid residues and is presented in Table 90B using the one-letter amino acid code.

TABLE 90B
Encoded NOV90 protein sequence.

(SEQ ID NO:212)

MNDDGKVNASSEGYFILVGFSNWPYLEVVLFVVILIFCLMTLIGNLFIIILTYLDSHLHTPLYFFLSNLS
FLDLCYTTSSIPQLLVSLWGVEKTISYAGCMVQLYFFLTLGTTECVLLVVMSYDRYAAVCRPLHYTVLMH
SRFCHLLAVASWVSGFTNPALHSSFTFWVPLCGHRQIDHFFCEVPALLRLSFVNTRENKLTLMITSSIFV
LLLLTLIFTSYGAIAQAVLRMQSTTGLQKVFGTCGAHHMVVSLFFIPAMCMYLQPPSGNSQDQGKFIALF
YTVVTPSLNPLIYTLRNKDVRGVVKRLRGWE

A search of sequence databases reveals that the NOV90 amino acid sequence has similarity with TREMBLNEW-ACC:CAC20478 OLFACTORY RECEPTOR—Homo sapiens. Public amino acid databases include the GenBank databases, SwissProt, PDB and PIR.

The disclosed NOV90 polypeptide has homology to the amino acid sequences shown in the BLASTP data listed in Table 90C.

TABLE 90C
BLAST results for NOV90

| Gene Index/ | | Length | Identity | Positives | |
|---|---|---|---|---|---|
| Identifier | Protein/Organism | (aa) | (%) | (%) | Expect |
| gi\|12054347\|emb\|CAC20478.1\| | olfactory | 311 | 281/311 | 293/311 | e−139 |
| (AJ302558) | receptor [Homo | | (90%) | (93%) | |
| | sapiens] | | | | |
| gi\|12054345\|emb\|CAC20477.1\| | olfactory | 311 | 280/311 | 293/311 | e−139 |
| (AJ302557) | receptor [Homo | | (90%) | (94%) | |
| | sapiens] | | | | |
| gi\|12140469\|emb\|CAC21440.1\| | olfactory | 311 | 280/311 | 292/311 | e−139 |
| (AJ302552) | receptor [Homo | | (90%) | (93%) | |
| | sapiens] | | | | |
| gi\|14423775\|sp\|O76001\| | OLFACTORY | 311 | 280/311 | 293/311 | e−139 |
| O2J3_HUMAN | RECEPTOR 2J3 | | (90%) | (94%) | |
| | (OLFACTORY | | | | |
| | RECEPTOR 6-6) | | | | |
| | (OR6-6) | | | | |
| gi\|18564769\|ref\|XP_ | similar to | 391 | 270/298 | 283/298 | e−136 |
| 069457.2\| | OLFACTORY | | (90%) | (94%) | |
| (XM_069457) | RECEPTOR 2J3 | | | | |
| | (OLFACTORY | | | | |
| | RECEPTOR 6-6) | | | | |
| | (OR6-6) (HS6M1-3) | | | | |
| | [Homo sapiens] | | | | |

Table 90D lists the domain descriptions from DOMAIN analysis results against NOV90. This indicates that the NOV90 sequence has properties similar to those of other proteins known to contain this domain.

TABLE 90D
Domain Analysis of NOV90

gnl|Pfam|pfam00001, 7tm_1, 7 transmembrane receptor
(rhodopsin family).
CD-Length = 254 residues, 100.0% aligned
Score = 89.7 bits (221), Expect = 2e−19

The disclosed NOV90 nucleic acid of the invention encoding a GPCR-like protein includes the nucleic acid whose sequence is provided in Table 90A or a fragment thereof. The invention also includes a mutant or variant nucleic acid any of whose bases may be changed from the corresponding base shown in Table 90A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV90 protein of the invention includes the GPCR-like protein whose sequence is provided in Table 90B. The invention also includes a mutant or variant protein any of whose residues may be changed from the corresponding residue shown in Table 90B while still encoding a protein that maintains its GPCR-like activities and physiological functions, or a functional fragment thereof. In the mutant or variant protein, up to about 5 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV90) may function as a member of a “GPCR family”. Therefore, the NOV90 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV90 nucleic acids and proteins of the invention are useful in potential therapeutic applications implicated in diseases including but not limited to various pathologies and disorders as indicated below. For example, a cDNA encoding the GPCR-like protein (NOV90) may be useful in gene therapy, and the GPCR-like protein (NOV90) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV nucleic acid encoding the GPCR-like protein of the invention, or fragments thereof, may further be useful in diagnostic applications, wherein the presence or amount of the nucleic acid or the protein are to be assessed.

NOV90 nucleic acids and polypeptides are further useful in the generation of antibodies that bind immuno-specifically to the novel NOV90 substances for use in therapeutic or diagnostic methods. These antibodies may be generated according to methods known in the art, using prediction from hydrophobicity charts, as described in the “Anti-NOVX Antibodies” section below. The disclosed NOV90 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV91

A disclosed NOV91 nucleic acid of 967 nucleotides (also referred to as CG57668-01) encoding a GPCR-like protein is shown in Table 91A. The start and stop codons are in bold letters.

TABLE 91A
NOV91 nucleotide sequence.

| | |
|---|---|
| GCATTTGCCCCAGTAGCTATGATTATAATTTGCAATGACAGCCACAGTGATTTCATCCTTCTGG | (SEQ ID NO:213) |
| GCTTCTCTAACAAGCCACATTTGGAGAAGATACTTTTTTGGATCATTTTTATTTTTTATTTTTT | |
| GACTCTTGCAGGAAATATGGTCATAGTTCTTGTGTCCTTGAAGGATCCAAAACTCCACATCCCT | |
| ATGTATTTCTTTCTTTCCAACCTTTCCTTGGTAGACCTCTGTTTGACCAGCAGCTGTGTTCCAC | |
| AGATGTTGATTAACTTCTGGGGCCCAGAAAAGACCATCAGCTACATTGGCTGTGCCATTCAACT | |
| CTATGTTTTTTTGTGGCTTGGGGCCACGGAATATGTCCTTCTTGTTGTCATGGCTGTGGATTGT | |
| TATGTAGCAGTGTGTCATCCACTGCAAAATACCATGATCATGCACCCAAAACTTTGTCTGCAGC | |
| TGGCTATCTTGGCATGGGGGACTGGCTTGGCCCAGTCTCTGATCCAGTCCCCTGCCACCCTCCG | |
| GTTACCCTTCTGCTCCCAGCGGATGGTGGATGATGTTGTTTGTGAAGTCCCAGCTCTGATTCAG | |
| CTCTCCAGTACTGATACTACCTACAGTGAAATTCAGATGTCTATCGCCAGTGTTGTCCTCCTGG | |
| TGATGCCCTTGATCATTATCCTTTCCTCTTCTGGTGCTATTGCTAAGGCTGTGCTGAGAATTAA | |
| GTCAACTGCAGGACAGAAGAAAGCATTTGGCACCTGCATCTCTCACCTTCTTGTGGTTTCTCTC | |
| TTTTATGGCACTGTCACAGGTGTCTACCTTCAACCAAAAAATCACTATCCTCATGAATGGGGCA | |
| AATTTCTCACTCTTTTCTACACTGTAGTAACCCCAACTCTTAATCCCCTCATCTACACTCTAAG | |
| GAACAAGGAGGTAAAGGGAGCACTAATAAGATTGGGGAGGAGGACCTGGGATTCCCAGAATAAC | |
| TAACAAG | |

The disclosed NOV91 polypeptide (SEQ ID NO:214) encoded by SEQ ID NO:213 has 314 amino acid residues and is presented in Table 91B using the one-letter amino acid code.

TABLE 91B
Encoded NOV91 protein sequence.

(SEQ ID NO:214)

MIIICNDSHSDFILLGFSNKPHLEKILFWIIFIFYFLTLAGNMVIVLVSLKDPKLHIPMYFFLSNLSLVD
LCLTSSCVPQMLINFWGPEKTISYIGCAIQLYVFLWLGATEYVLLVVMAVDCYVAVCHPLQNTMIMHPKL
CLQLAILAWGTGLAQSLIQSPATLRLPFCSQRMVDDVVCEVPALIQLSSTDTTYSEIQMSIASVVLLVMP
LIIILSSSGAIAKAVLRIKSTAGQKKAFGTCISHLLVVSLFYGTVTGVYLQPKNHYPHEWGKFLTLFYTV
VTPTLNPLIYTLRNKEVKGALIRLGRRTWDSQNN

A search of sequence databases reveals that the NOV91 amino acid sequence has 188/303 (62%) identity and 232/303 (76%) similarity with SWISSNEW-ACC:Q15062 OLFACTORY RECEPTOR 2H3 (OLFACTORY RECEPTOR-LIKE PROTEIN FAT11)-Homo sapiens. Public amino acid databases include the GenBank databases, SwissProt, PDB and PIR.

The disclosed NOV91 polypeptide has homology to the amino acid sequences shown in the BLASTP data listed in Table 91C.

TABLE 91C
BLAST results for NOV91

| Gene Index/ | | Length | Identity | Positives | |
|---|---|---|---|---|---|
| Identifier | Protein/Organism | (aa) | (%) | (%) | Expect |
| gi\|17464345\|ref\|XP_ | similar to | 470 | 217/221 | 219/221 | e−111 |
| 069459.1\| | olfactory | | (98%) | (98%) | |
| (XM_069459) | receptor [Homo | | | | |
| | sapiens] | | | | |
| gi\|18565396\|ref\|XP_ | 216/221 (97%), | 289 | 216/221 | 218/221 | e−111 |
| 094938.1\| | Positives = | | (97%) | (97%) | |
| (XM_094938 | 218/221 (97%) | | | | |
| gi\|12231029\|sp\|Q15062\| | Olfactory | 316 | 188/303 | 232/303 | 6e−99 |
| O2H3_HUMAN | receptor 2H3 | | (62%) | (76%) | |
| | (Olfactory | | | | |
| | receptor-like | | | | |
| | protein FAT11) | | | | |
| gi\|9798922\|gb\|AAF98753.1\| | olfactory | 303 | 187/293 | 228/293 | 2e−97 |
| AF211941_1 | receptor [Homo | | (63%) | (76%) | |
| (AF211941) | sapiens] | | | | |
| gi\|14423783\|sp\|O95918\| | Olfactory | 312 | 187/302 | 230/302 | 3e−97 |
| O2H2_HUMAN | receptor 2H2 | | (61%) | (75%) | |
| | (Hs6M1-12) | | | | |

Table 91D lists the domain descriptions from DOMAIN analysis results against NOV91. This indicates that the NOV91 sequence has properties similar to those of other proteins known to contain this domain.

TABLE 91D
Domain Analysis of NOV91

gnl|Pfam|pfam00001, 7tm_1, 7 transmembrane receptor
(rhodopsin family).
CD-Length = 254 residues, 100.0% aligned
Score = 98.6 bits (244), Expect = 5e−22

The disclosed NOV91 nucleic acid of the invention encoding a GPCR-like protein includes the nucleic acid whose sequence is provided in Table 91A or a fragment thereof. The invention also includes a mutant or variant nucleic acid any of whose bases may be changed from the corresponding base shown in Table 91A while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5 percent of the bases may be so changed.

The disclosed NOV91 protein of the invention includes the GPCR-like protein whose sequence is provided in Table 91B. The invention also includes a mutant or variant protein any of whose residues may be changed from the corresponding residue shown in Table 91B while still encoding a protein that maintains its GPCR-like activities and physiological functions, or a functional fragment thereof. In the mutant or variant protein, up to about 38 percent of the residues may be so changed.

The invention further encompasses antibodies and antibody fragments, such as F_{ab} or (F_{ab})₂, that bind immunospecifically to any of the proteins of the invention.

The above defined information for this invention suggests that this GPCR-like protein (NOV91) may function as a member of a “GPCR family”. Therefore, the NOV91 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV91 nucleic acids and proteins of the invention are useful in potential therapeutic applications implicated in diseases including but not limited to various pathologies and disorders as indicated below. For example, a cDNA encoding the GPCR-like protein (NOV91) may be useful in gene therapy, and the GPCR-like protein (NOV91) may be useful when administered to a subject in need thereof. By way of nonlimiting example, the compositions of the present invention will have efficacy for treatment of patients suffering from CNS disorders, brain disorders including epilepsy, eating disorders, schizophrenia, ADD; cancer; heart disease; inflammation and autoimmune disorders including Crohn's disease, IBD, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, pancreatic disorders including pancreatic insufficiency and cancer; and prostate disorders including prostate cancer, or other pathologies or conditions. The NOV nucleic acid encoding the GPCR-like protein of the invention, or fragments thereof, may further be useful in diagnostic applications, wherein the presence or amount of the nucleic acid or the protein are to be assessed.

NOV91 nucleic acids and polypeptides are further useful in the generation of antibodies that bind immuno-specifically to the novel NOV91 substances for use in therapeutic or diagnostic methods. These antibodies may be generated according to methods known in the art, using prediction from hydrophobicity charts, as described in the “Anti-NOVX Antibodies” section below. The disclosed NOV91 proteins have multiple hydrophilic regions, each of which can be used as an immunogen. These novel proteins can be used in assay systems for functional analysis of various human disorders, which will help in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of Such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, 27, 29, and 31, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 25, 27, 29, and 31 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 5, 7, 9, 11, 13, 15, 17, 19, 21, 23, 25, 27, 29, and 31, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, 27, 29, and 31, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 17, 19, 21, 23, 25, 27, 29, or 31 is one that is sufficiently complementary to the nucleotide sequence shown SEQ ID NOS:1, 3, 5, 7, 9, 11, 13, 15, 17, 19, 21, 23, 25, 27, 29, or 31 that it can hydrogen bond with little or no mismatches to the nucleotide sequence shown SEQ ID NOS:1, 3, 5, 7, 9, 11, 13, 15, 17, 19, 21, 23, 25, 27, 29, and 31, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, 27, 29, and 31, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17, 19, 21, 23, 25, 27, 29, or 31; or an anti-sense strand nucleotide sequence of SEQ ID NOS:1, 3, 5, 7, 9, 11, 13, 15, 17, 19, 21, 23, 25, 27, 29, or 31; or of a naturally occurring mutant of SEQ ID NOS:1, 3, 5, 7, 9, 11, 13, 15, 17, 19, 21, 23, 25, 27, 29, and 31.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 21, 23, 25, 27, 29, or 31, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17, 19, 21, 23, 25, 27, 29, and 31 due to degeneracy of the genetic code and thus encode the same NOVX proteins as that encoded by the nucleotide sequences shown in SEQ ID NOS:1, 3, 5, 7, 9, 11, 13, 15, 17, 19, 21, 23, 25, 27, 29, and 31. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 26, 28, 30, or 32.

In addition to the human NOVX nucleotide sequences shown in SEQ ID NOS:1, 3, 5, 7, 9, 11, 13, 15, 17, 19, 21, 23, 25, 27, 29, and 31, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 17, 19, 21, 23, 25, 27, 29, and 31 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 25, 27, 29, and 31. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and p111. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 5, 7, 9, 11, 13, 15, 17, 19, 21, 23, 25, 27, 29, and 31, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, 27, 29, and 31, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 17, 19, 21, 23, 25, 27, 29, and 31, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17, 19, 21, 23, 25, 27, 29, and 31, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26, 28, 30, or 32. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:1, 3, 5, 7, 9, 11, 13, 15, 17, 19, 21, 23, 25, 27, 29, and 31 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26, 28, 30, and 32. Preferably, the protein encoded by the nucleic acid molecule is at least about 60% homologous to SEQ ID NOS:2, 4, 6, 8, 10, 12, 14, 16, 18, 20, 22, 24, 26, 28, 30, and 32; more preferably at least about 70% homologous SEQ ID NOS:2, 4, 6, 8, 10, 12, 14, 16, 18, 20, 22, 24, 26, 28, 30, or 32; still more preferably at least about 80% homologous to SEQ ID NOS:2, 4, 6, 8, 10, 12, 14, 16, 18, 20, 22, 24, 26, 28, 30, or 32; even more preferably at least about 90% homologous to SEQ ID NOS:2, 4, 6, 8, 10, 12, 14, 16, 18, 20, 22, 24, 26, 28, 30, or 32; and most preferably at least about 95% homologous to SEQ ID NOS:2, 4, 6, 8, 10, 12, 14, 16, 18, 20, 22, 24, 26, 28, 30, or 32.

An isolated nucleic acid molecule encoding an NOVX protein homologous to the protein of SEQ ID NOS:2, 4, 6, 8, 10, 12, 14, 16, 18, 20, 22, 24, 26, 28, 30, or 32 can be created by introducing one or more nucleotide substitutions, additions or deletions into the nucleotide sequence of SEQ ID NOS:1, 3, 5, 7, 9, 11, 13, 15, 17, 19, 21, 23, 25, 27, 29, and 31, such that one or more amino acid substitutions, additions or deletions are introduced into the encoded protein.

Mutations can be introduced into SEQ ID NOS:1, 3, 5, 7, 9, 11, 13, 15, 17, 19, 21, 23, 25, 27, 29, and 31 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 5, 7, 9, 11, 13, 15, 17, 19, 21, 23, 25, 27, 29, and 31, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, 27, 29, and 31, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26, 28, 30, or 32, or antisense nucleic acids complementary to an NOVX nucleic acid sequence of SEQ ID NOS:1, 3, 5, 7, 9, 11, 13, 15, 17, 19, 21, 23, 25, 27, 29, and 31, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 17, 19, 21, 23, 25, 27, 29, and 31). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. USA. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 26, 28, 30, or 32. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, or 32 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 26, 28, 30, or 32) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 26, 28, 30, or 32. In other embodiments, the NOVX protein is substantially homologous to SEQ ID NOS:2, 4, 6, 8, 10, 12, 14, 16, 18, 20, 22, 24, 26, 28, 30, or 32, and retains the functional activity of the protein of SEQ ID NOS:2, 4, 6, 8, 10, 12, 14, 16, 18, 20, 22, 24, 26, 28, 30, or 32, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 26, 28, 30, or 32, and retains the functional activity of the NOVX proteins of SEQ ID NOS:2, 4, 6, 8, 10, 12, 14, 16, 18, 20, 22, 24, 26, 28, 30, or 32.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 21, 23, 25, 27, 29, and 31.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 26, 28, 30, or 32, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, Fab, F_{ab′} and F(_{ab′})₂ fragments, and an Fat) expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Alai. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse mycloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine mycloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or mycloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol com26S protease regulatory subunit 4 g agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human r cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucinc zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the VHl and VI domains of one fragment are forced to pair with the complementary V_{H} and V_{L} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FCγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors Of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend oil such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Flerskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SFS9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of Suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the tern as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce nonhuman transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17, 19, 21, 23, 25, 27, 29, and 31 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17, 19, 21, 23, 25, 27, 29, and 31), but more preferably, is a non-human homologue of a human NOVX gene. For example, a mouse homologue of human NOVX gene of SEQ ID NOS:1, 3, 5, 7, 9, 11, 13, 15, 17, 19, 21, 23, 25, 27, 29, and 31 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69:915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaccutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. USA. 90: 6909; Erb, et al., 1994. Proc. Natl. Acadc Sci. USA. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting Such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17, 19, 21, 23, 25, 27, 29, and 31, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, 27, 29, and 31 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogeniomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17, 19, 21, 23, 25, 27, 29, and 31, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-denisity arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, el al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tiblech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect.

One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table 13A shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table 13B shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE EL1A
PCR Primers for Exon Linking

| | | SEQ | | |
|---|---|---|---|---|
| NOVX | | ID | | ID |
| Clone | Primer 1 (5′-3′) | NO | Primer 2 (5′-3′ | NO |
| 4b | GAAGGTTTCCCTGGGCGTTCCTT | 215 | GTGAGGTGCAGGCAAAACCAATGATT | 216 |
| 15 | GACCCCAAGAGCCTTAATGACTCTAGA | 217 | CTGTCCGTCGTCCTTCAGAGTCAT | 218 |
| 21 | CAACCAAGAGGCAAGAGG | 219 | CTCCATGAGACTCAGTGAATAAGA | 220 |
| 23 | CTGCCTTCTGCCTTATGCCA | 221 | TTAAGTTCTAGGGTACATGTGCACAAC | 222 |
| 24 | GCCTGGTCCTGCTGACTG | 223 | CCGCATCAGCCTAGGGGTACTAGAGAT | 224 |
| 42b | CTGTGCACTGTTGGTGGGAATATAAAA | 225 | TCTGGTGGTTAAGATAAAACACAAGTCA | 226 |
| 47 | TTCGGCTGCTGCTGACCAT | 227 | CCTGGTAGCCTCAAAGCTTCTTACTTC | 228 |
| 57 | ATGGCTGCCGAGAACTCCTCCTC | 229 | TCAAGAAAAGCTTATTCTGGAAAAGGTT | 230 |
| | | | CTCTTC | |
| 58 | AACCCCTGCTGTCATCCTTCTC | 231 | GCTACAAAAGGTTTCTTTCTGATCT | 232 |
| | | | GC | |
| 60c | GTAAACATTTGGCCAGCTTGGTTTG | 233 | CAGCTGCCTGGCTAACTCCTATAACAC | 234 |
| 62b | AAGGTGCTGAAATAGCAATGACAAGAG | 235 | CAGAGTCTCTCCCTAGCTCCCCAG | 236 |
| 67b | ATACCCCACGTTCCGCTATGAGATT | 237 | GTGTCACGTCGAGTGGTTGGTG | 238 |
| 69b | CACATAGTCTTGGCTCCAGTTTCGT | 239 | CTAAAGTTTTATTCCAATCAGTGTTTTTTTT | 240 |
| | | | TCC | |
| 81b | GAATGATGCCCTTTTGCCACAA | 241 | CTATGAACTCAATTCCAAAAATAATTTACAC | 242 |
| | | | CTG | |

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

### Quantitative Expression Analysis of Clones in Various Tissues and Cells

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 FIT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:128s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version 1 for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_Screening_Panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Flyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100M non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, the isolated CD8 lymphocytes were activated for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, PWM was used at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, DMEM or RPMI (as recommended by the ATCC) was used, with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI-comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=AutoimmuLnity

Syn=Syiiovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omenturn Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigr-a=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21y, parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no nieuropathology

Control (Path)=Control brains; pateint not demenlted but showing sever AD-like pathology

SupTemporal Ctx=Superior Temporal Cortex

Inif Temporal Ctx=Inferior Tremporal Cortex

NOV1

Expression of NOV1(CG57602-01) was assessed using the primer-probe set Ag3294, described in Table AA. Results of the RTQ-PCR runs are shown in Tables AB, AC and AD.

TABLE AA
Probe Name Ag3294

| Primers | Sequences | Length | Start Position | SEQ ID NO |
|---|---|---|---|---|
| Forward | 5′-ccttttgtgggttccatagtc-3′ | 21 | 6686 | 243 |
| Probe | TET-5′-tttgtccccaagggccttccagt-3′-TAMRA | 23 | 6707 244 | |
| Reverse | 5′-aaaagcaggtattcaacaagca-3′ | 22 | 6758 | 245 |

TABLE AB
CNS_neurodegeneration_v1.0

| | Rel. Exp. (%) | | Rel. Exp. (%) |
|---|---|---|---|
| | Ag3294, Run | | Ag3294, Run |
| Tissue Name | 210062302 | Tissue Name | 210062302 |
| AD 1 Hippo | 0.0 | Control (Path) 3 Temporal | 0.0 |
| | | Ctx | |
| AD 2 Hippo | 0.0 | Control (Path) 4 Temporal | 0.0 |
| | | Ctx | |
| AD 3 Hippo | 0.0 | AD 1 Occipital Ctx | 0.0 |
| AD 4 Hippo | 0.0 | AD 2 Occipital Ctx | 0.0 |
| | | (Missing) | |
| AD 5 Hippo | 0.1 | AD 3 Occipital Ctx | 0.0 |
| AD 6 Hippo | 0.0 | AD 4 Occipital Ctx | 100.0 |
| Control 2 Hippo | 0.0 | AD 5 Occipital Ctx | 0.0 |
| Control 4 Hippo | 0.0 | AD 6 Occipital Ctx | 0.0 |
| Control (Path) 3 Hippo | 0.0 | Control 1 Occipital Ctx | 0.0 |
| AD 1 Temporal Ctx | 0.0 | Control 2 Occipital Ctx | 0.0 |
| AD 2 Temporal Ctx | 0.0 | Control 3 Occipital Ctx | 0.0 |
| AD 3 Temporal Ctx | 0.0 | Control 4 Occipital Ctx | 0.0 |
| AD 4 Temporal Ctx | 0.0 | Control (Path) 1 Occipital | 0.1 |
| | | Ctx | |
| AD 5 Inf Temporal Ctx | 0.1 | Control (Path) 2 Occipital | 0.0 |
| | | Ctx | |
| AD 5 Sup Temporal Ctx | 0.1 | Control (Path) 3 Occipital | 0.0 |
| | | Ctx | |
| AD 6 Inf Temporal Ctx | 0.1 | Control (Path) 4 Occipital | 0.0 |
| | | Ctx | |
| AD 6 Sup Temporal Ctx | 0.1 | Control 1 Parietal Ctx | 0.0 |
| Control 1 Temporal Ctx | 0.0 | Control 2 Parietal Ctx | 0.1 |
| Control 2 Temporal Ctx | 0.0 | Control 3 Parietal Ctx | 0.0 |
| Control 3 Temporal Ctx | 0.0 | Control (Path) 1 Parietal | 0.0 |
| | | Ctx | |
| Control 3 Temporal Ctx | 0.0 | Control (Path) 2 Parietal | 0.0 |
| | | Ctx | |
| Control (Path) 1 Temporal | 0.1 | Control (Path) 3 Parietal | 0.0 |
| Ctx | | Ctx | |
| Control (Path) 2 Temporal | 0.0 | Control (Path) 4 Parietal | 0.0 |
| Ctx | | Ctx | |

TABLE AC
General_screening_panel_v1.4

| | Rel. Exp. (%) | | Rel. Exp. (%) |
|---|---|---|---|
| | Ag3294, Run | | Ag3294, Run |
| Tissue Name | 215669619 | Tissue Name | 215669619 |
| Adipose | 0.0 | Renal ca. TK-10 | 0.0 |
| Melanoma* Hs688(A).T | 0.0 | Bladder | 4.9 |
| Melanoma* Hs688(B).T | 0.9 | Gastric ca. (liver met.) NCI- | 11.1 |
| | | N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 1.4 | Colon ca. SW480 | 7.1 |
| Squamous cell carcinoma | 0.0 | Colon ca.* (SW480 met) | 1.2 |
| SCC-4 | | SW620 | |
| Testis Pool | 20.0 | Colon ca. HT29 | 3.2 |
| Prostate ca.* (bone met) | 5.1 | Colon ca. HCT-116 | 2.3 |
| PC-3 | | | |
| Prostate Pool | 3.6 | Colon ca. CaCo-2 | 0.7 |
| Placenta | 2.6 | Colon cancer tissue | 0.0 |
| Uterus Pool | 0.0 | Colon ca. SW1116 | 0.5 |
| Ovarian ca. OVCAR-3 | 17.9 | Colon ca. Colo-205 | 0.0 |
| Ovarian ca. SK-OV-3 | 1.4 | Colon ca. SW-48 | 0.0 |
| Ovarian ca. OVCAR-4 | 0.0 | Colon Pool | 6.0 |
| Ovarian ca. OVCAR-5 | 23.7 | Small Intestine Pool | 2.5 |
| Ovarian ca. IGROV-1 | 6.2 | Stomach Pool | 2.6 |
| Ovarian ca. OVCAR-8 | 1.4 | Bone Marrow Pool | 0.0 |
| Ovary | 1.3 | Fetal Heart | 0.0 |
| Breast ca. MCF-7 | 8.0 | Heart Pool | 2.6 |
| Breast ca. MDA-MB-231 | 4.1 | Lymph Node Pool | 3.6 |
| Breast ca. BT 549 | 4.0 | Fetal Skeletal Muscle | 1.1 |
| Breast ca. T47D | 38.2 | Skeletal Muscle Pool | 2.0 |
| Breast ca. MDA-N | 1.3 | Spleen Pool | 0.9 |
| Breast Pool | 10.5 | Thymus Pool | 10.2 |
| Trachea | 7.2 | CNS cancer (glio/astro) U87- | 0.0 |
| | | MG | |
| Lung | 0.0 | CNS cancer (glio/astro) U- | 1.1 |
| | | 118-MG | |
| Fetal Lung | 54.3 | CNS cancer (neuro;met) SK- | 100.0 |
| | | N-AS | |
| Lung ca. NCI-N417 | 0.0 | CNS cancer (astro) SF-539 | 0.0 |
| Lung ca. LX-1 | 2.6 | CNS cancer (astro) SNB-75 | 0.0 |
| Lung ca. NCI-H146 | 0.7 | CNS cancer (glio) SNB-19 | 2.4 |
| Lung ca. SHP-77 | 0.0 | CNS cancer (glio) SF-295 | 33.9 |
| Lung ca. A549 | 13.3 | Brain (Amygdala) Pool | 8.4 |
| Lung ca. NCI-H526 | 2.4 | Brain (cerebellum) | 23.8 |
| Lung ca. NCI-H23 | 71.7 | Brain (fetal) | 5.1 |
| Lung ca. NCI-H460 | 10.0 | Brain (Hippocampus) Pool | 26.2 |
| Lung ca. HOP-62 | 1.4 | Cerebral Cortex Pool | 13.7 |
| Lung ca NCI-H522 | 34.6 | Brain (Substantia nigra) Pool | 20.7 |
| Liver | 3.1 | Brain (Thalamus) Pool | 26.1 |
| Fetal Liver | 2.5 | Brain (whole) | 15.8 |
| Liver ca. HepG2 | 0.0 | Spinal Cord Pool | 15.4 |
| Kidney Pool | 6.1 | Adrenal Gland | 5.6 |
| Fetal Kidney | 18.7 | Pituitary gland Pool | 2.4 |
| Renal ca. 786-0 | 1.3 | Salivary Gland | 0.0 |
| Renal ca. A498 | 7.3 | Thyroid (female) | 0.0 |
| Renal ca. ACHN | 2.9 | Pancreatic ca. CAPAN2 | 5.5 |
| Renal ca. UO-31 | 0.0 | Pancreas Pool | 5.3 |

TABLE AD
Panel 4D

| | Rel. Exp. (%) | | Rel. Exp. (%) |
|---|---|---|---|
| | Ag3294, Run | | Ag3294, Run |
| Tissue Name | 165014460 | Tissue Name | 165014460 |
| Secondary Th1 act | 4.5 | HUVEC IL-1beta | 0.0 |
| Secondary Th2 act | 0.0 | HUVEC IFN gamma | 3.1 |
| Secondary Tr1 act | 3.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 4.3 | Lung Microvascular EC none | 0.0 |
| Primary Th1 act | 0.0 | Lung Microvascular EC TNFalpha + | 0.0 |
| | | IL-1beta | |
| Primary Th2 act | 0.0 | Microvascular Dermal EC none | 3.6 |
| Primary Tr1 act | 0.0 | Microsvasular Dermal EC | 0.0 |
| | | TNFalpha + IL-1beta | |
| Primary Th1 rest | 0.0 | Bronchial epithelium TNFalpha + | 0.0 |
| | | IL1beta | |
| Primary Th2 rest | 4.6 | Small airway epithelium none | 0.0 |
| Primary Tr1 rest | 0.0 | Small airway epithelium TNFalpha + | 0.0 |
| | | IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNFalpha + | 3.5 |
| act | | IL-1beta | |
| CD8 lymphocyte act | 0.0 | Astrocytes rest | 4.5 |
| Secondary CD8 lymphocyte | 0.0 | Astrocytes TNFalpha + IL-1beta | 0.0 |
| rest | | | |
| Secondary CD8 lymphocyte | 0.0 | KU-812 (Basophil) rest | 10.2 |
| act | | | |
| CD4 lymphocyte none | 3.7 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNFalpha + IL-1beta | |
| LAK cells IL-2 | 0.0 | Liver cirrhosis | 26.2 |
| LAK cells IL-2 + IL-12 | 0.0 | Lupus kidney | 0.0 |
| LAK cells IL-2 + IFN gamma | 0.0 | NCI-H292 none | 6.5 |
| LAK cells IL-2 + IL-18 | 0.0 | NCI-H292 IL-4 | 12.6 |
| LAK cells PMA/ionomycin | 0.0 | NCI-H292 IL-9 | 3.8 |
| NK Cells IL-2 rest | 0.0 | NCI-H292 IL-13 | 12.2 |
| Two Way MLR 3 day | 12.9 | NCI-H292 IFN gamma | 3.8 |
| Two Way MLR 5 day | 0.0 | HPAEC none | 0.0 |
| Two Way MLR 7 day | 6.7 | HPAEC TNF alpha + IL-1 beta | 0.0 |
| PBMC rest | 0.0 | Lung fibroblast none | 2.4 |
| PBMC PWM | 0.0 | Lung fibroblast TNF alpha + IL-1 | 7.2 |
| | | beta | |
| PBMC PHA-L | 0.0 | Lung fibroblast IL-4 | 8.9 |
| Ramos (B cell) none | 0.0 | Lung fibroblast IL-9 | 0.0 |
| Ramos (B cell) ionomycin | 7.6 | Lung fibroblast IL-13 | 0.0 |
| B lymphocytes PWM | 0.0 | Lung fibroblast IFN gamma | 0.0 |
| B lymphocytes CD40L and | 7.3 | Dermal fibroblast CCD1070 rest | 0.0 |
| IL-4 | | | |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL-1 | 0.0 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 6.3 |
| Monocytes rest | 4.2 | IBD Crohn's | 0.0 |
| Monocytes LPS | 0.0 | Colon | 100.0 |
| Macrophages rest | 2.1 | Lung | 43.2 |
| Macrophages LPS | 0.0 | Thymus | 7.6 |
| HUVEC none | 2.3 | Kidney | 3.4 |
| HUVEC starved | 3.3 | | |

CNS_neurodegeneration_v1.0 Summary: Ag3294 Results from one experiment with the CG57602-01 gene are not included. The amp plot indicates that there were experimental difficulties with this run.

General_screening_panel_v1.4 Summary: Ag3294 Expression of the CG57602-01 gene is highest in a CNS cancer cell line (CT=31.8). Therefore, expression of this gene may be used to distinguish this sample from the other samples on this panel. Interestingly, expression of this gene is higher in fetal lung (CT=32.7) than in adult lung (CT=40), suggesting that expression of this gene may be used to distinguish adult and fetal lung. Additionally, this gene is expressed at a moderate level in two lung cancer cell lines suggesting a possible role in lung cancer.

In addition, this gene is expressed at low levels in several regions of the central nervous system, including cerebellum, hippocampus, cerebral cortex, substantia nigra, thalamus and spinal cord (CTs=33.7-35). Therefore, therapeutic modulation of the activity of this gene may be of benefit in the treatment of CNS disorders such as Alzheimer's disease, Parkinson's disease, epilepsy, multiple sclerosis, schizophrenia and depression.

Panel 4D Summary: Ag3294 Expression of the CG57602-01 gene is highest in colon (CT=33.3), with low but significant expression also detected in lung (CT=34.5). Therefore, expression of this gene may be used to distinguish colon and lung from the other tissues oil this panel. Furthermore, expression of this gene is decreased in colon samples from patients with IBD colitis and Crohn's disease relative to normal colon. Therefore, therapeutic modulation of the activity of the protein encoded by this gene, using small molecule drugs, antibodies, or protein therapeutics, may be useful in the treatment of inflammatory bowel disease.

NOV2

Expression of NOV2 (CG57558-01) was assessed using the primer-probe set Ag3285, described in Table BA.

TABLE BA
Probe Name Ag3285

| Primers | Sequences | Length | Start Position | SEQ ID NO |
|---|---|---|---|---|
| Forward | 5′-gacaacaaacaagcaaacacaa-3′ | 22 | 11 | 246 |
| Probe | TET-5′-accttccacttccttctggtcctgct-3′-TAMRA | 26 | 62 | 247 |
| Reverse | 5′-gcaggagaggaggaagaagag-3′ | 21 | 89 | 248 |

CNS_neurodegeneration_v1.0 Summary: Ag3285 Expression of the CG57558-01 gene is low/undetectable (CTs>35) across all of the samples on this panel (data not shown).

Panel 1.3D Summary: Ag3285 Expression of the CG57558-01 gene is low/undetectable (CTs>35) across all of the samples on this panel (data not shown).

Panel 4D Summary: Ag3285 Expression of the CG57558-01 gene is low/undetectable (CTs>35) across all of the samples on this panel (data not shown).

NOV3

Expression of NOV3 (CG57560-01) was assessed using the primer-probe sets Ag3286 and Ag663, described in Tables CA and CB. Results of the RTQ-PCR runs are shown in Tables CC, CD, CE and CF.

TABLE CA
Probe Name Ag3286

| Primers | Sequences | Length | Start Position | SEQ ID NO |
|---|---|---|---|---|
| Forward | 5′-gaggaggtggaagaagatgatt-3′ | 22 | 764 | 249 |
| Probe | TET-5′-agaatctcttccgcaagatcctggct-3′-TAMRA | 26 | 801 | 250 |
| Reverse | 5′-catcccaatatggagagtcaaa-3′ | 22 | 839 | 251 |

TABLE CB
Probe Name Ag663

| Primers | Sequences | Length | Start Position | SEQ ID NO |
|---|---|---|---|---|
| Forward | 5′-acctttctatgaggaggtggaa-3′ | 22 | 754 | 252 |
| Probe | TET-5′-tgagaaccatgataagaatctcttccg-3′-TAMRA | 27 | 787 | 253 |
| Reverse | 5′-gtcaccagccaggatcttg-3′ | 19 | 814 | 254 |

TABLE CC
CNS_neurodegeneration_v1.0

| | Rel. Exp. | | Rel. Exp. |
|---|---|---|---|
| | (%) Ag3286, | | (%) Ag3286, |
| Tissue Name | Run 210060841 | Tissue Name | Run 210060841 |
| AD 1 Hippo | 12.2 | Control (Path) 3 | 1.3 |
| | | Temporal Ctx | |
| AD 2 Hippo | 41.8 | Control (Path) 4 | 27.7 |
| | | Temporal Ctx | |
| AD 3 Hippo | 6.3 | AD 1 Occipital Ctx | 9.0 |
| AD 4 Hippo | 5.7 | AD 2 Occipital Ctx | 0.0 |
| | | (Missing) | |
| AD 5 hippo | 61.1 | AD 3 Occipital Ctx | 0.8 |
| AD 6 Hippo | 58.6 | AD 4 Occipital Ctx | 14.9 |
| Control 2 Hippo | 68.3 | AD 5 Occipital Ctx | 11.6 |
| Control 4 Hippo | 2.6 | AD 6 Occipital Ctx | 60.7 |
| Control (Path) 3 | 1.2 | Control 1 Occipital | 0.2 |
| Hippo | | Ctx | |
| AD 1 Temporal Ctx | 7.5 | Control 2 Occipital | 88.3 |
| | | Ctx | |
| AD 2 Temporal Ctx | 39.0 | Control 3 Occipital | 8.2 |
| | | Ctx | |
| AD 3 Temporal Ctx | 1.9 | Control 4 Occipital | 0.8 |
| | | Ctx | |
| AD 4 Temporal Ctx | 17.6 | Control (Path) 1 | 75.3 |
| | | Occipital Ctx | |
| AD 5 Inf Temporal | 88.9 | Control (Path) 2 | 4.4 |
| Ctx | | Occipital Ctx | |
| AD 5 Sup Temporal | 34.9 | Control (Path) 3 | 0.2 |
| Ctx | | Occipital Ctx | |
| AD 6 Inf Temporal | 37.6 | Control (Path) 4 | 6.7 |
| Ctx | | Occipital Ctx | |
| AD 6 Sup Temporal | 47.3 | Control 1 Parietal | 1.4 |
| Ctx | | Ctx | |
| Control 1 Temporal | 1.2 | Control 2 Parietal | 15.2 |
| Ctx | | Ctx | |
| Control 2 Temporal | 70.7 | Control 3 Parietal | 11.3 |
| Ctx | | Ctx | |
| Control 3 Temporal | 14.3 | Control (Path) 1 | 100.0 |
| Ctx | | Parietal Ctx | |
| Control 4 Temporal | 3.5 | Control (Path) 2 | 15.4 |
| Ctx | | Parietal Ctx | |
| Control (Path) 1 | 87.7 | Control (Path) 3 | 0.7 |
| Temporal Ctx | | Parietal Ctx | |
| Control (Path) 2 | 39.2 | Control (Path) 4 | 33.9 |
| Temporal Ctx | | Parietal Ctx | |

TABLE CD
General_screening_panel_v1.4

| | Rel. Exp. | | Rel. Exp. |
|---|---|---|---|
| | (%) Ag3286, | | (%) Ag3286, |
| Tissue Name | Run 216512997 | Tissue Name | Run 216512997 |
| Adipose | 0.0 | Renal ca. TK-10 | 0.1 |
| Melanoma* | 0.0 | Bladder | 0.0 |
| Hs688(A).T | | | |
| Melanoma* | 0.0 | Gastric ca. (liver met.) | 0.1 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.3 | Colon ca. SW480 | 0.6 |
| MEL-5 | | | |
| Squamous cell | 0.0 | Colon ca.* (SW480 | 0.9 |
| carcinoma SCC-4 | | met) SW620 | |
| Testis Pool | 0.2 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 3.1 |
| Placenta | 0.0 | Colon cancer tissue | 0.0 |
| Uterus Pool | 0.0 | Colon ca. SW1116 | 0.0 |
| Ovarian ca. | 0.1 | Colon ca. Colo-205 | 0.1 |
| OVCAR-3 | | | |
| Ovarian ca. SK-OV-3 | 0.0 | Colon ca. SW-48 | 0.0 |
| Ovarian ca. | 0.0 | Colon Pool | 0.1 |
| OVCAR-4 | | | |
| Ovarian ca. | 0.1 | Small Intestine Pool | 0.0 |
| OVCAR-5 | | | |
| Ovarian ca. IGROV- | 0.1 | Stomach Pool | 0.0 |
| 1 | | | |
| Ovarian ca. | 0.0 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 0.0 | Lymph Node Pool | 0.0 |
| MB-231 | | | |
| Breast ca. BT 549 | 0.1 | Fetal Skeletal Muscle | 0.0 |
| Breast ca. T47D | 0.1 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.1 | CNS cancer (glio/astro) | 0.0 |
| | | U87-MG | |
| Lung | 0.0 | CNS cancer (glio/astro) | 0.1 |
| | | U-118-MG | |
| Fetal Lung | 0.0 | CNS cancer | 2.3 |
| | | (neuro;met) SK-N-AS | |
| Lung ca. NCI-N417 | 7.2 | CNS cancer (astro) SF- | 100.0 |
| | | 539 | |
| Lung ca. LX-1 | 0.1 | CNS cancer (astro) | 0.2 |
| | | SNB-75 | |
| Lung ca. NCI-H146 | 1.9 | CNS cancer (glio) | 0.0 |
| | | SNB-19 | |
| Lung ca. SHP-77 | 0.5 | CNS cancer (glio) SF- | 0.0 |
| | | 295 | |
| Lung ca. A549 | 0.1 | Brain (Amygdala) Pool | 10.4 |
| Lung ca. NCI-H526 | 0.6 | Brain (cerebellum) | 1.2 |
| Lung ca. NCI-H23 | 0.3 | Brain (fetal) | 35.6 |
| Lung ca. NCI-H460 | 0.3 | Brain (Hippocampus) | 20.7 |
| | | Pool | |
| Lung ca. HOP-62 | 0.0 | Cerebral Cortex Pool | 20.4 |
| Lung ca. NCI-H522 | 0.1 | Brain (Substantia nigra) | 13.4 |
| | | Pool | |
| Liver | 0.0 | Brain (Thalamus) Pool | 22.2 |
| Fetal Liver | 0.0 | Brain (whole) | 19.1 |
| Liver ca. HepG2 | 0.0 | Spinal Cord Pool | 1.5 |
| Kidney Pool | 0.0 | Adrenal Gland | 0.0 |
| Fetal Kidney | 0.0 | Pituitary gland Pool | 1.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.1 | Pancreas Pool | 0.0 |

TABLE CE
Panel 1.1

| | Rel. Exp. | | Rel. Exp. |
|---|---|---|---|
| | (%) Ag663, | | (%) Ag663, |
| Tissue Name | Run 109559269 | Tissue Name | Run 109559269 |
| Adrenal gland | 0.0 | Renal ca. UO-31 | 0.0 |
| Bladder | 0.0 | Renal ca. RXF 393 | 0.0 |
| Brain (amygdala) | 23.2 | Liver | 0.0 |
| Brain (cerebellum) | 1.8 | Liver (fetal) | 0.0 |
| Brain (hippocampus) | 82.9 | Liver ca. | 0.0 |
| | | (hepatoblast) HepG2 | |
| Brain (substantia | 15.6 | Lung | 0.0 |
| nigra) | | | |
| Brain (thalamus) | 32.8 | Lung (fetal) | 0.0 |
| Cerebral Cortex | 90.8 | Lung ca. (non-s. cell) | 0.0 |
| | | HOP-62 | |
| Brain (fetal) | 100.0 | Lung ca. (large | 0.0 |
| | | cell)NCI-H460 | |
| Brain (whole) | 41.8 | Lung ca. (non-s. cell) | 0.0 |
| | | NCI-H23 | |
| glio/astro U-118-MG | 0.0 | Lung ca. (non-s. cl) | 0.0 |
| | | NCI-H522 | |
| astrocytoma SF-539 | 0.0 | Lung ca. (non-sm. | 0.0 |
| | | cell) A549 | |
| astrocytoma SNB-75 | 0.0 | Lung ca. (s. cell var.) | 0.0 |
| | | SHP-77 | |
| astrocytoma SW1783 | 0.0 | Lung ca. (small cell) | 0.0 |
| | | LX-1 | |
| glioma U251 | 0.0 | Lung ca. (small cell) | 4.3 |
| | | NCI-H69 | |
| glioma SF-295 | 0.0 | Lung ca. (squam.) | 0.0 |
| | | SW 900 | |
| glioma SNB-19 | 0.0 | Lung ca. (squam.) | 16.6 |
| | | NCI-H596 | |
| glio/astro U87-MG | 0.0 | Lymph node | 0.0 |
| neuro*; met SK-N-AS | 3.8 | Spleen | 0.0 |
| Mammary gland | 0.0 | Thymus | 0.0 |
| Breast ca. BT-549 | 0.0 | Ovary | 0.0 |
| Breast ca. MDA-N | 0.0 | Ovarian ca. IGROV- | 0.0 |
| | | 1 | |
| Breast ca.* (pl. ef) | 0.0 | Ovarian ca. OVCAR- | 0.0 |
| T47D | | 3 | |
| Breast ca.* (pl. ef) | 0.0 | Ovarian ca. OVCAR- | 0.0 |
| MCF-7 | | 4 | |
| Breast ca.* (pl. ef) | 0.0 | Ovarian ca. OVCAR- | 0.0 |
| MDA-MB-231 | | 5 | |
| Small intestine | 0.0 | Ovarian ca. OVCAR- | 0.0 |
| | | 8 | |
| Colorectal | 0.0 | Ovarian ca.* (ascites) | 0.0 |
| | | SK-OV-3 | |
| Colon ca. HT29 | 0.0 | Pancreas | 0.0 |
| Colon ca. CaCo-2 | 2.9 | Pancreatic ca. | 0.0 |
| | | CAPAN 2 | |
| Colon ca. HCT-15 | 0.0 | Pituitary gland | 5.7 |
| Colon ca. HCT-116 | 0.0 | Placenta | 0.0 |
| Colon ca. HCC-2998 | 0.0 | Prostate | 0.0 |
| Colon ca. SW480 | 0.0 | Prostate ca.* (bone | 0.0 |
| | | met) PC-3 | |
| Colon ca.* SW620 | 0.0 | Salivary gland | 0.0 |
| (SW480 met) | | | |
| Stomach | 0.0 | Trachea | 0.0 |
| Gastric ca. (liver met) | 0.0 | Spinal cord | 0.3 |
| NCI-N87 | | | |
| Heart | 0.0 | Testis | 0.0 |
| Skeletal muscle (Fetal) | 0.0 | Thyroid | 0.0 |
| Skeletal muscle | 0.0 | Uterus | 0.0 |
| Endothelial cells | 0.0 | Melanoma M14 | 0.0 |
| Heart (Fetal) | 0.0 | Melanoma LOX | 0.0 |
| | | IMVI | |
| Kidney | 0.0 | Melanoma UACC-62 | 0.0 |
| Kidney (fetal) | 0.0 | Melanoma SK-MEL- | 0.0 |
| | | 28 | |
| Renal ca. 786-0 | 0.0 | Melanoma* (met) | 0.0 |
| | | SK-MEL-5 | |
| Renal ca. A498 | 0.0 | Melanoma | 0.0 |
| | | Hs688(A).T | |
| Renal ca. ACHN | 0.0 | Melanoma* (met) | 0.0 |
| | | Hs688(B).T | |
| Renal ca. TK-10 | 0.0 | | |

TABLE CF
Panel 4D

| | Rel. Exp. (%) | | Rel. Exp. (%) |
|---|---|---|---|
| | Ag3286, Run | | Ag3286, Run |
| Tissue Name | 164633940 | Tissue Name | 164633940 |
| Secondary Th1 act | 5.5 | HUVEC IL-1beta | 0.0 |
| Secondary Th2 act | 4.2 | HUVEC IFN gamma | 0.0 |
| Secondary Tr1 act | 15.3 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 33.2 | Lung Microvascular EC | 0.0 |
| | | TNFalpha + IL-1beta | |
| Primary Th2 act | 24.7 | Microvascular Dermal EC | 0.0 |
| | | none | |
| Primary Tr1 act | 16.2 | Microsvasular Dermal EC | 0.0 |
| | | TNFalpha + IL-1beta | |
| Primary Th1 rest | 0.0 | Bronchial epithelium | 5.1 |
| | | TNFalpha + IL1beta | |
| Primary Th2 rest | 0.0 | Small airway epithelium | 0.0 |
| | | none | |
| Primary Tr1 rest | 4.8 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 27.0 | Coronery artery SMC | 0.0 |
| lymphocyte act | | TNFalpha + IL-1beta | |
| CD8 lymphocyte act | 10.4 | Astrocytes rest | 3.5 |
| Secondary CD8 | 18.6 | Astrocytes TNFalpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 7.9 | CCD1106 (Keratinocytes) | 0.0 |
| | | TNFalpha + IL-1beta | |
| LAK cells IL-2 | 0.0 | Liver cirrhosis | 17.6 |
| LAK cells IL-2 + IL-12 | 0.0 | Lupus kidney | 0.0 |
| LAK cells IL-2 + IFN | 7.6 | NCI-H292 none | 0.0 |
| gamma | | | |
| LAK cells IL-2 + IL-18 | 26.8 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 4.4 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 11.3 | Lung fibroblast TNF alpha + | 0.0 |
| | | IL-1 beta | |
| PBMC PHA-L | 14.3 | Lung fibroblast IL-4 | 0.0 |
| Ramos (B cell) none | 0.0 | Lung fibroblast IL-9 | 2.8 |
| Ramos (B cell) | 0.0 | Lung fibroblast IL-13 | 0.0 |
| ionomycin | | | |
| B lymphocytes PWM | 100.0 | Lung fibroblast IFN | 0.0 |
| | | gamma | |
| B lymphocytes CD40L | 8.7 | Dermal fibroblast | 0.0 |
| and IL-4 | | CCD1070 rest | |
| EOL-1 dbcAMP | 0.0 | Dermal fibroblast | 0.0 |
| | | CCD1070 TNFalpha | |
| EOL-1 dbcAMP | 0.0 | Dermal fibroblast | 0.0 |
| PMA/ionomycin | | CCD1070 IL-1 beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 6.7 |
| Monocytes LPS | 0.0 | Colon | 49.3 |
| Macrophages rest | 1.0 | Lung | 17.9 |
| Macrophages LPS | 6.2 | Thymus | 0.0 |
| HUVEC none | 0.0 | Kidney | 1.5 |
| HUVEC starved | 0.0 | | |

CNS_neurodegeneration_v1.0 Summary: Ag3286 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3286 The CG57560-01 gene encodes a protein with homology to calmodulin-binding protein kinases. Expression of this gene is highest in the CNS cancer cell line SF-539 (CT=23.9). Strikingly, this gene is also expressed at moderate to high levels almost exclusively in the CNS; expression is detected amygdala, cerebellum, thalamus, hippocampus, cerebral cortex, substantia nigra and spinal cord. Thus, expression of this gene may be used to distinguish brain from the other samples on this panel.

Furthermore, this brain-specific expression pattern suggests that this gene may play a role in central nervous system disorders such as Alzheimer's disease, Parkinson's disease, epilepsy, multiple sclerosis, schizophrenia and depression.

In addition, moderate expression of this gene is seen in the pituitary gland (CT=30.6). Therefore, this gene may play a role in endocrine disorders, diabetes, obesity, and growth disorders.

Furthermore, this gene is expressed in most of the lung cancer cell lines on this panel but at undetectable levels in the normal adult lung or fetal tissues. Hence, expression of the CG57560-01 gene might be used as a diagnostic marker for lung cancer. In addition, therapeutic modulation of the activity of this gene, using small molecule drugs, antibodies or protein therapeutics, may be of benefit in the treatment of lung cancer.

Panel 1.1 Summary: Ag663 The CG57560-01 gene is expressed at high levels in all of the CNS samples examined including amygdala, cerebellum, thalamus, hippocampus, cerebral cortex, spinal cord, and the substantia nigra. Highest expression of this gene is seen in the fetal brain sample (CT=22.1). These results are consistent with what is observed in General_screening_panel_v1.4; please see this panel for a discussion of the relevance of this gene in the central nervous system.

Panel 4D Summary: Ag3286 Expression of the CG57560-01 gene is highest in pokeweed mitogen stimulated peripheral blood mononuclear cells (CT=32.7) Expression of this transcript in B cells suggests that this gene may be involved in rheumatic disease including rheumatoid arthritis, lupus, osteoarthritis, and hyperproliferative B cell disorders.

In addition, low but significant expression of this gene is seen in activated T cells and memory T cells, but not resting T cells, suggesting that CG57560-01 gene expression may play a role in T cell mediated diseases such as autoimmunity or delayed type hypersensitivity reactions.

NOV4

Expression of NOV4a (CG57547-01) and NOV4b CG57547-02 was assessed using the primer-probe sets Ag3690, Ag531, Ag534 and Ag535, described in Tables DA, DB, DC and DD. Results of the RTQ-PCR runs are shown in Tables DE, DF, DG, DH, DI, DJ and DK. Please note that the CG57547-02 variant is not recognized by primer-probe set Ag3690.

TABLE DA
Probe Name Ag3690

| Primers | Sequences | Length | Start Position | SEQ ID NO |
|---|---|---|---|---|
| Forward | 5′-agacgtcaaacagggaaatctt-3′ | 22 | 1744 | 255 |
| Probe | TET-5′-cctccaggatataagatcactctgattga-3′-TAMRA | 29 | 1766 | 256 |
| Reverse | 5′-tctgtaggttcctcccatgag-3′ | 21 | 1817 | 257 |

TABLE DB
Probe Name Ag531

| Primers | Sequenccs | Length | Start Position | SEQ ID NO |
|---|---|---|---|---|
| Forward | 5′-tccaggatataagatcactctgattgata-3′ | 29 | 1768 | 258 |
| Probe | TET-5′-aggttcctcccatgagatattcaataacaagtcct-3′-TAMRA | 35 | 1798 | 259 |
| Reverse | 5′-aacgtttcctagtataggtgcatctg-3′ | 26 | 1834 | 260 |

TABLE DC
Probe Name Ag534

| Primers | Sequences | Length | Start Position | SEQ ID NO |
|---|---|---|---|---|
| Forward | 5′-tcagtgatacaattgccataatttcttt-3′ | 28 | 3039 | 261 |
| Probe | TET-5′-tttgctccaaatcttagtccaaatccaagaa-3′-TAMRA | 32 | 3068 | 262 |
| Reverse | 5′-aaaaacatgattatcatatgcatttgc-3′ | 27 | 3110 | 263 |

TABLE DD
Probe Name Ag535

| Primers | Sequences | Length | Start Position | SEQ ID NO |
|---|---|---|---|---|
| Forward | 5′-tttacaagtgaaggcaatttccaa-3′ | 24 | 3562 | 264 |
| Probe | TET-5′-agccataataaaatgataacgctggtacttccatacaat-3′-TAMRA | 39 | 3587 | 265 |
| Reverse | 5′-gaggcagaactggtttctcatga-3′ | 23 | 3628 | 266 |

TABLE DE
CNS_neurodegeneration_v1.0

| | Rel. Exp. (%) Ag3690, |
|---|---|
| Tissue Name | Run 211144703 |
| AD 1 Hippo | 17.9 |
| AD 2 Hippo | 20.0 |
| AD 3 Hippo | 17.2 |
| AD 4 Hippo | 6.1 |
| AD 5 Hippo | 97.3 |
| AD 6 Hippo | 62.4 |
| Control 2 Hippo | 23.2 |
| Control 4 Hippo | 12.4 |
| Control (Path) 3 Hippo | 22.4 |
| AD 1 Temporal Ctx | 34.2 |
| AD 2 Temporal Ctx | 37.1 |
| AD 3 Temporal Ctx | 12.2 |
| AD 4 Temporal Ctx | 27.0 |
| AD 5 Inf Temporal Ctx | 85.3 |
| AD 5 Sup Temporal Ctx | 87.7 |
| AD 6 Inf Temporal Ctx | 80.7 |
| AD 6 Sup Temporal Ctx | 82.9 |
| Control 1 Temporal Ctx | 7.9 |
| Control 2 Temporal Ctx | 23.7 |
| Control 3 Temporal Ctx | 19.3 |
| Control 4 Temporal Ctx | 9.1 |
| Control (Path) 1 Temporal Ctx | 56.6 |
| Control (Path) 2 Temporal Ctx | 43.2 |
| Control (Path) 3 Temporal Ctx | 4.3 |
| Control (Path) 4 Temporal Ctx | 39.5 |
| AD 1 Occipital Ctx | 38.2 |
| AD 2 Occipital Ctx (Missing) | 0.0 |
| AD 3 Occipital Ctx | 11.8 |
| AD 4 Occipital Ctx | 28.5 |
| AD 5 Occipital Ctx | 25.0 |
| AD 6 Occipital Ctx | 45.4 |
| Control 1 Occipital Ctx | 5.1 |
| Control 2 Occipital Ctx | 51.1 |
| Control 3 Occipital Ctx | 24.8 |
| Control 4 Occipital Ctx | 11.7 |
| Control (Path) 1 Occipital Ctx | 100.0 |
| Control (Path) 2 Occipital Ctx | 22.5 |
| Control (Path) 3 Occipital Ctx | 2.8 |
| Control (Path) 4 Occipital Ctx | 29.7 |
| Control 1 Parietal Ctx | 14.0 |
| Control 2 Parietal Ctx | 62.4 |
| Control 3 Parietal Ctx | 18.3 |
| Control (Path) 1 Parietal Ctx | 74.7 |
| Control (Path) 2 Parietal Ctx | 33.4 |
| Control (Path) 3 Parietal Ctx | 5.1 |
| Control (Path) 4 Parietal Ctx | 48.6 |

TABLE DF
General_screening_panel_v1.4

| | Rel. Exp. (%) | | Rel. Exp. (%) |
|---|---|---|---|
| | Ag3690, Run | | Ag3690, Run |
| Tissue Name | 217130999 | Tissue Name | 217130999 |
| Adipose | 12.6 | Renal ca. TK-10 | 26.8 |
| Melanoma* Hs688(A).T | 32.5 | Bladder | 24.1 |
| Melanoma* Hs688(B).T | 31.0 | Gastric ca. (liver met.) NCI- | 61.1 |
| | | N87 | |
| Melanoma* M14 | 53.6 | Gastric ca. KATO III | 38.2 |
| Melanoma* LOXIMVI | 20.0 | Colon ca. SW-948 | 6.1 |
| Melanoma* SK-MEL-5 | 57.0 | Colon ca. SW480 | 22.5 |
| Squamous cell carcinoma | 11.6 | Colon ca.* (SW480 met) | 35.4 |
| SCC-4 | | SW620 | |
| Testis Pool | 10.1 | Colon ca. HT29 | 20.7 |
| Prostate ca.* (bone met) | 39.2 | Colon ca. HCT-116 | 37.1 |
| PC-3 | | | |
| Prostate Pool | 11.3 | Colon ca. CaCo-2 | 66.0 |
| Placenta | 3.8 | Colon cancer tissue | 16.7 |
| Uterus Pool | 8.1 | Colon ca. SW1116 | 5.1 |
| Ovarian ca. OVCAR-3 | 26.1 | Colon ca. Colo-205 | 8.0 |
| Ovarian ca. SK-OV-3 | 34.9 | Colon ca. SW-48 | 3.7 |
| Ovarian ca. OVCAR-4 | 3.7 | Colon Pool | 22.4 |
| Ovarian ca. OVCAR-5 | 40.1 | Small Intestine Pool | 20.4 |
| Ovarian ca. IGROV-1 | 11.7 | Stomach Pool | 11.0 |
| Ovarian ca. OVCAR-8 | 15.1 | Bone Marrow Pool | 9.2 |
| Ovary | 15.6 | Fetal Heart | 27.0 |
| Breast ca. MCF-7 | 100.0 | Heart Pool | 13.3 |
| Breast ca. MDA-MB-231 | 20.9 | Lymph Node Pool | 27.5 |
| Breast ca. BT 549 | 52.1 | Fetal Skeletal Muscle | 5.6 |
| Breast ca. T47D | 54.0 | Skeletal Muscle Pool | 15.8 |
| Breast ca. MDA-N | 16.7 | Spleen Pool | 15.9 |
| Breast Pool | 24.3 | Thymus Pool | 24.5 |
| Trachea | 10.6 | CNS cancer (glio/astro) U87- | 63.3 |
| | | MG | |
| Lung | 6.7 | CNS cancer (glio/astro) U- | 54.7 |
| | | 118-MG | |
| Fetal Lung | 32.3 | CNS cancer (neuro, met) SK- | 24.1 |
| | | N-AS | |
| Lung ca. NCI-N417 | 5.6 | CNS cancer (astro) SF-539 | 27.0 |
| Lung ca. LX-1 | 34.6 | CNS cancer (astro) SNB-75 | 55.1 |
| Lung ca. NCI-H146 | 2.9 | CNS cancer (glio) SNB-19 | 14.9 |
| Lung ca. SHP-77 | 19.8 | CNS cancer (glio) SF-295 | 66.4 |
| Lung ca. A549 | 35.8 | Brain (Amygdala) Pool | 4.6 |
| Lung ca. NCI-H526 | 4.7 | Brain (cerebellum) | 7.9 |
| Lung ca. NCI-H23 | 47.3 | Brain (fetal) | 14.0 |
| Lung ca. NCI-H460 | 27.9 | Brain (Hippocampus) Pool | 6.4 |
| Lung ca. HOP-62 | 10.1 | Cerebral Cortex Pool | 7.4 |
| Lung ca. NCI-H522 | 24.0 | Brain (Substantia nigra) Pool | 5.0 |
| Liver | 1.4 | Brain (Thalamus) Pool | 10.7 |
| Fetal Liver | 25.2 | Brain (whole) | 6.5 |
| Liver ca. HepG2 | 12.1 | Spinal Cord Pool | 8.0 |
| Kidney Pool | 29.9 | Adrenal Gland | 7.2 |
| Fetal Kidney | 31.2 | Pituitary gland Pool | 4.5 |
| Renal ca. 786-0 | 26.1 | Salivary Gland | 2.3 |
| Renal ca. A498 | 15.4 | Thyroid (female) | 5.2 |
| Renal ca. ACHN | 14.3 | Pancreatic ca. CAPAN2 | 15.3 |
| Renal ca. UO-31 | 12.2 | Pancreas Pool | 27.4 |

TABLE DG
Panel 1.1

| | Rel. Exp. (%) | | Rel. Exp. (%) |
|---|---|---|---|
| | Ag534, Run | | Ag534, Run |
| Tissue Name | 111162676 | Tissue Name | 111162676 |
| Adrenal gland | 21.2 | Renal ca. UO-31 | 6.3 |
| Bladder | 57.8 | Renal ca. RXF 393 | 4.4 |
| Brain (amygdala) | 3.7 | Liver | 59.5 |
| Brain (cerebellum) | 59.9 | Liver (fetal) | 13.8 |
| Brain (hippocampus) | 15.0 | Liver ca. (hepatoblast) | 13.7 |
| | | HepG2 | |
| Brain (substantia nigra) | 21.5 | Lung | 5.6 |
| Brain (thalamus) | 7.6 | Lung (fetal) | 6.6 |
| Cerebral Cortex | 12.9 | Lung ca. (non-s. cell) HOP- | 50.3 |
| | | 62 | |
| Brain (fetal) | 17.9 | Lung ca. (large cell) NCI- | 47.6 |
| | | H460 | |
| Brain (whole) | 9.9 | Lung ca. (non-s. cell) NCI- | 27.7 |
| | | H23 | |
| glio/astro U-118-MG | 12.3 | Lung ca. (non-s. cl) NCI- | 74.2 |
| | | H522 | |
| astrocytoma SF-539 | 27.0 | Lung ca. (non-sm. cell) | 32.1 |
| | | A549 | |
| astrocytoma SNB-75 | 7.6 | Lung ca. (s. cell var.) SHP- | 7.6 |
| | | 77 | |
| astrocytoma SW1783 | 4.9 | Lung ca. (small cell) LX-1 | 52.5 |
| glioma U251 | 18.0 | Lung ca. (small cell) NCI- | 39.2 |
| | | H69 | |
| glioma SF-295 | 31.2 | Lung ca. (squam.) SW 900 | 12.6 |
| glioma SNB-19 | 35.6 | Lung ca. (squam.) NCI- | 34.6 |
| | | H596 | |
| glio/astro U87-MG | 35.4 | Lymph node | 10.4 |
| neuro*; met SK-N-AS | 32.5 | Spleen | 6.4 |
| Mammary gland | 10.7 | Thymus | 13.1 |
| Breast ca. BT-549 | 8.0 | Ovary | 2.9 |
| Breast ca. MDA-N | 32.1 | Ovarian ca. IGROV-1 | 21.9 |
| Breast ca.* (pl. ef) T47D | 62.9 | Ovarian ca. OVCAR-3 | 11.7 |
| Breast ca.* (pl. ef) MCF-7 | 98.6 | Ovarian ca. OVCAR-4 | 4.5 |
| Breast ca.* (pl. ef) MDA- | 7.4 | Ovarian ca. OVCAR-5 | 42.3 |
| MB-231 | | | |
| Small intestine | 24.8 | Ovarian ca. OVCAR-8 | 51.4 |
| Colorectal | 3.1 | Ovarian ca.* (ascites) SK- | 28.1 |
| | | OV-3 | |
| Colon ca. HT29 | 24.7 | Pancreas | 39.5 |
| Colon ca. CaCo-2 | 48.0 | Pancreatic ca. CAPAN 2 | 4.3 |
| Colon ca.HCT-15 | 15.3 | Pituitary gland | 28.7 |
| Colon ca.HCT-116 | 21.3 | Placenta | 27.5 |
| Colon ca. HCC-2998 | 53.2 | Prostate | 23.0 |
| Colon ca. SW480 | 5.2 | Prostate ca.* (bone met) | 32.1 |
| | | PC-3 | |
| Colon ca.* SW620 (SW480 | 44.4 | Salivary gland | 41.2 |
| met) | | | |
| Stomach | 13.0 | Trachea | 15.3 |
| Gastric ca. (liver met) NCI- | 67.4 | Spinal cord | 14.1 |
| N87 | | | |
| Heart | 44.1 | Testis | 9.8 |
| Skeletal muscle (Fetal) | 6.1 | Thyroid | 19.2 |
| Skeletal muscle | 54.0 | Uterus | 17.1 |
| Endothelial cells | 17.2 | Melanoma M14 | 26.6 |
| Heart (Fetal) | 6.5 | Melanoma LOX IMVI | 5.3 |
| Kidney | 100.0 | Melanoma UACC-62 | 15.5 |
| Kidney (fetal) | 30.4 | Melanoma SK-MEL-28 | 80.7 |
| Renal ca. 786-0 | 15.9 | Melanoma* (met) SK- | 39.5 |
| | | MEL-5 | |
| Renal ca. A498 | 16.4 | Melanoma Hs688(A).T | 14.9 |
| Renal ca. ACHN | 11.3 | Melanoma* (met) | 16.3 |
| | | Ms688(B).T | |
| Renal ca. TK-10 | 25.9 | | |

TABLE DH
Panel 1.2

| | Rel. Exp. (%) | | Rel. Exp. (%) |
|---|---|---|---|
| | Ag535, Run | | Ag535, Run |
| Tissue Name | 112162329 | Tissue Name | 112162329 |
| Endothelial cells | 10.2 | Renal ca. 786-0 | 14.6 |
| Heart (Fetal) | 1.5 | Renal ca. A498 | 21.8 |
| Pancreas | 94.0 | Renal ca. RXF 393 | 5.5 |
| Pancreatic ca. CAPAN 2 | 8.4 | Renal ca. ACHN | 13.4 |
| Adrenal Gland | 41.5 | Renal ca. UO-31 | 5.8 |
| Thyroid | 29.1 | Renal ca. TK-10 | 22.2 |
| Salivary gland | 47.0 | Liver | 56.3 |
| Pituitary gland | 48.3 | Liver (fetal) | 20.6 |
| Brain (fetal) | 17.1 | Liver ca. (hepatoblast) | 11.3 |
| | | HepG2 | |
| Brain (whole) | 41.8 | Lung | 14.0 |
| Brain (amygdala) | 9.3 | Lung (fetal) | 20.7 |
| Brain (cerebellum) | 20.0 | Lung ca. (small cell) LX-I | 48.3 |
| Brain (hippocampus) | 20.7 | Lung ca. (small cell) NCI- | 27.4 |
| | | H69 | |
| Brain (thalamus) | 17.6 | Lung ca. (s. cell var ) SHP- | 5.6 |
| | | 77 | |
| Cerebral Cortex | 11.9 | Lung ca. (large cell) NCI- | 61.1 |
| | | H460 | |
| Spinal cord | 12.0 | Lung ca. (non-sm. cell) | 25.9 |
| | | A549 | |
| glio/astro U87-MG | 45.1 | Lung ca. (non-s. cell) NCI- | 22.4 |
| | | H23 | |
| glio/astro U-118-MG | 20.0 | Lung ca. (non-s. cell) HOP- | 34.9 |
| | | 62 | |
| astrocytoma SW1783 | 6.3 | Lung ca. (non-s. cl) NCI- | 100.0 |
| | | H522 | |
| neuro*; met SK-N-AS | 34.4 | Lung ca (squam.) SW 900 | 15.8 |
| astrocytoma SF-539 | 22.2 | Lung ca. (squam.) NCI- | 40.3 |
| | | H596 | |
| astrocytoma SNB-75 | 9.6 | Mammary gland | 30.4 |
| glioma SNB-19 | 31.0 | Breast ca.* (pl. ef) MCF-7 | 70.7 |
| glioma U251 | 24.7 | Breast ca.* (pl. ef) MDA- | 6.6 |
| | | MB-231 | |
| glioma SF-295 | 27.7 | Breast ca.* (pl. ef) T47D | 37.9 |
| Heart | 49.3 | Breast ca. BT-549 | 16.6 |
| Skeletal Muscle | 48.6 | Breast ca. MDA-N | 31.4 |
| Bone marrow | 23.2 | Ovary | 2.5 |
| Thymus | 17.7 | Ovarian ca. OVCAR-3 | 25.5 |
| Spleen | 22.5 | Ovarian ca. OVCAR-4 | 5.6 |
| Lymph node | 33.0 | Ovarian ca. OVCAR-5 | 45.1 |
| Colorectal Tissue | 1.5 | Ovarian ca. OVCAR-8 | 23.3 |
| Stomach | 24.7 | Ovarian ca. IGROV-1 | 27.2 |
| Small intestine | 34.4 | Ovarian ca. (ascitcs) SK- | 33.9 |
| | | OV-3 | |
| Colon ca. SW480 | 5.0 | Uterus | 21.2 |
| Colon ca.* SW620 (SW480 | 25.3 | Placenta | 60.7 |
| met) | | | |
| Colon ca. HT29 | 20.3 | Prostate | 25.3 |
| Colon ca. HCT-116 | 20.4 | Prostate ca.* (bone met) PC- | 59.9 |
| | | 3 | |
| Colon ca. CaCo-2 | 39.8 | Testis | 37.1 |
| Colon ca. Tissue | 2.5 | Melanoma Hs688(A).T | 14.5 |
| (ODO3866) | | | |
| Colon ca. HCC-2998 | 54.7 | Melanoma* (met) | 18.7 |
| | | Hs688(B).T | |
| Gastric ca.* (liver met) NCI- | 69.3 | Melanoma UACC-62 | 19.1 |
| N87 | | | |
| Bladder | 43.8 | Melanoma M14 | 25.2 |
| Trachea | 13.2 | Melanoma LOX IMVI | 4.2 |
| Kidney | 48.3 | Melanoma* (met) SK-MEL- | 50.0 |
| | | 5 | |
| Kidney (fetal) | 51.8 | | |

TABLE DI
Panel 1.3D

| | Rel. Exp. (%) | | Rel. Exp. (%) |
|---|---|---|---|
| | Ag531, Run | | Ag531, Run |
| Tissue Name | 165974812 | Tissue Name | 165974812 |
| Liver adenocarcinoma | 22.1 | Kidney (fetal) | 17.0 |
| Pancreas | 6.8 | Renal ca. 786-0 | 15.7 |
| Pancreatic ca. CAPAN2 | 19.3 | Renal ca. A498 | 22.4 |
| Adrenal gland | 3.8 | Renal ca. RXF 393 | 30.8 |
| Thyroid | 3.8 | Renal ca. ACHN | 10.7 |
| Salivary gland | 10.4 | Renal ca. UO-31 | 10.7 |
| Pituitary gland | 15.8 | Renal ca. TK-10 | 12.3 |
| Brain (fetal) | 11.8 | Liver | 10.9 |
| Brain (whole) | 30.6 | Liver (fetal) | 10.0 |
| Brain (amygdala) | 16.8 | Liver ca. (hepatoblast) | 20.2 |
| | | HepG2 | |
| Brain (cerebellum) | 45.4 | Lung | 8.5 |
| Brain (hippocampus) | 20.3 | Lung (fetal) | 18.7 |
| Brain (substantia nigra) | 7.6 | Lung ca. (small cell) LX-I | 49.0 |
| Brain (thalamus) | 21.9 | Lung ca. (small cell) NCI- | 50.3 |
| | | H69 | |
| Cerebral Cortex | 9.0 | Lung ca. (s. cell var.) SHP- | 23.5 |
| | | 77 | |
| Spinal cord | 27.5 | Lung ca. (large cell) NCI- | 11.7 |
| | | H460 | |
| glio/astro U87-MG | 40.1 | Lung ca. (non-sm. cell) | 10.2 |
| | | A549 | |
| glio/astro U-118-MG | 26.4 | Lung ca. (non-s. cell) NCI- | 30.4 |
| | | H23 | |
| astrocytoma SW1783 | 21.3 | Lung ca. (non-s. cell) HOP- | 13.5 |
| | | 62 | |
| neuro*; met SK-N-AS | 13.0 | Lung ca. (non-s. cl) NCI- | 12.9 |
| | | H522 | |
| astrocytoma SF-539 | 68.3 | Lung ca. (squam.) SW 900 | 22.5 |
| astrocytoma SNB-75 | 12.3 | Lung ca. (squam.) NCI- | 49.0 |
| | | H596 | |
| glioma SNB-19 | 65.1 | Mammary gland | 5.9 |
| glioma U251 | 29.9 | Breast ca.* (pl. ef) MCF-7 | 100.0 |
| glioma SF-295 | 11.8 | Breast ca.* (pl. ef) MDA- | 7.7 |
| | | MB-231 | |
| Heart (fetal) | 3.0 | Breast ca.* (pl. ef) T47D | 18.9 |
| Heart | 9.7 | Breast ca. BT-549 | 14.2 |
| Skeletal muscle (fetal) | 1.2 | Breast ca. MDA-N | 8.6 |
| Skeletal muscle | 11.0 | Ovary | 1.5 |
| Bone marrow | 19.5 | Ovarian ca. OVCAR-3 | 11.3 |
| Thymus | 16.2 | Ovarian ca. OVCAR-4 | 7.5 |
| Spleen | 11.3 | Ovarian ca. OVCAR-5 | 34.6 |
| Lymph node | 25.0 | Ovarian ca. OVCAR-8 | 20.9 |
| Colorectal | 10.5 | Ovarian ca. IGROV-1 | 11.0 |
| Stomach | 11.1 | Ovarian ca.* (ascites) SK- | 41.5 |
| | | OV-3 | |
| Small intestine | 17.1 | Uterus | 9.9 |
| Colon ca. SW480 | 8.7 | Placenta | 26.4 |
| Colon ca.* SW620(SW480 | 24.8 | Prostate | 5.7 |
| met) | | | |
| Colon ca. HT29 | 14.3 | Prostate ca.* (bone met)PC- | 31.0 |
| | | 3 | |
| Colon ca. HCT-116 | 13.0 | Testis | 9.0 |
| Colon ca. CaCo-2 | 50.7 | Melanoma Hs688(A).T | 11.7 |
| Colon ca. tissue(ODO3866) | 18.7 | Melanoma* (met) | 21.9 |
| | | Hs688(B).T | |
| Colon ca. HCC-2998 | 36.1 | Melanoma UACC-62 | 20.4 |
| Gastric ca.* (liver met) NCI- | 48.6 | Melanoma M14 | 31.4 |
| N87 | | | |
| Bladder | 22.4 | Melanoma LOX IMVI | 3.7 |
| Trachea | 3.1 | Melanoma* (met) SK-MEL- | 19.5 |
| | | 5 | |
| Kidney | 13.0 | Adipose | 11.0 |

TABLE DJ
Panel 4.1D

| | Rel. Exp. (%) | | Rel. Exp. (%) |
|---|---|---|---|
| | Ag3690, Run | | Ag3690, Run |
| Tissue Name | 169988048 | Tissue Name | 169988048 |
| Secondary Th1 act | 69.7 | HUVEC IL-1beta | 22.7 |
| Secondary Th2 act | 77.4 | HUVEC IFN gamma | 33.9 |
| Secondary Tr1 act | 79.6 | HUVEC TNF alpha + IFN gamma | 25.5 |
| Secondary Th1 rest | 28.7 | HUVEC TNF alpha + IL4 | 29.3 |
| Secondary Th2 rest | 39.2 | HUVEC IL-11 | 14.8 |
| Secondary Tr1 rest | 38.7 | Lung Microvascular EC none | 45.4 |
| Primary Th1 act | 64.2 | Lung Microvascular EC TNFalpha + | 43.8 |
| | | IL-1beta | |
| Primary Th2 act | 79.6 | Microvascular Dermal EC none | 35.4 |
| Primary Tr1 act | 76.3 | Microsvasular Dermal EC | 34.6 |
| | | TNFalpha + IL-1beta | |
| Primary Th1 rest | 45.1 | Bronchial epithelium TNFalpha + | 57.4 |
| | | IL1beta | |
| Primary Th2 rest | 38.2 | Small airway epithelium none | 12.4 |
| Primary Tr1 rest | 64.6 | Small airway epithelium TNFalpha + | 23.2 |
| | | IL-1beta | |
| CD45RA CD4 lymphocyte | 52.9 | Coronery artery SMC rest | 26.8 |
| act | | | |
| CD45RO CD4 lymphocyte | 84.1 | Coronery artery SMC TNFalpha + | 20.3 |
| act | | IL-1beta | |
| CD8 lymphocyte act | 75.8 | Astrocytes rest | 20.4 |
| Secondary CD8 lymphocyte | 74.2 | Astrocytes TNFalpha + IL-1beta | 22.1 |
| rest | | | |
| Secondary CD8 lymphocyte | 33.0 | KU-812 (Basophil) rest | 45.1 |
| act | | | |
| CD4 lymphocyte none | 26.6 | KU-812 (Basophil) | 85.9 |
| | | PMA/ionomycin | |
| 2ry Th1/Th2/Tr1_anti-CD95 | 49.7 | CCD1106 (Keratinocytes) none | 39.8 |
| CH11 | | | |
| LAK cells rest | 57.8 | CCD1106 (Keratinocytes) | 33.0 |
| | | TNFalpha + IL-1beta | |
| LAK cells IL-2 | 66.0 | Liver cirrhosis | 18.2 |
| LAK cells IL-2 + IL-12 | 51.8 | NCI-H292 none | 31.9 |
| LAK cells IL-2 + IFN gamma | 68.3 | NCI-H292 IL-4 | 38.2 |
| LAK cells IL-2 + IL-18 | 68.3 | NCI-H292 IL-9 | 64.6 |
| LAK cells PMA/ionomycin | 56.6 | NCI-H292 IL-13 | 37.9 |
| NK Cells IL-2 rest | 66.4 | NCI-H292 IFN gamma | 50.0 |
| Two Way MLR 3 day | 80.1 | HPAEC none | 27.7 |
| Two Way MLR 5 day | 59.5 | HPAEC TNF alpha + IL-1 beta | 49.0 |
| Two Way MLR 7 day | 44.1 | Lung fibroblast none | 50.7 |
| PBMC rest | 22.5 | Lung fibroblast TNF alpha + IL-1 | 18.3 |
| | | beta | |
| PBMC PWM | 74.7 | Lung fibroblast IL-4 | 38.7 |
| PBMC PHA-L | 59.9 | Lung fibroblast IL-9 | 52.5 |
| Ramos (B cell) none | 100.0 | Lung fibroblast IL-13 | 37.9 |
| Ramos (B cell) ionomycin | 88.9 | Lung fibroblast IFN gamma | 41.8 |
| B lymphocytes PWM | 50.0 | Dermal fibroblast CCD1070 rest | 52.9 |
| B lymphocytes CD40L and | 88.9 | Dermal fibroblast CCD1070 TNF | 84.7 |
| IL-4 | | alpha | |
| EOL-1 dbcAMP | 54.3 | Dermal fibroblast CCD1070 IL-1 | 27.7 |
| | | beta | |
| EOL-1 dbcAMP | 73.2 | Dermal fibroblast IFN gamma | 30.1 |
| PMA/ionomycin | | | |
| Dendritic cells none | 59.9 | Dermal fibroblast IL-4 | 80.1 |
| Dendritic cells LPS | 53.2 | Dermal Fibroblasts rest | 37.6 |
| Dendritic cells anti-CD40 | 61.1 | Neutrophils TNFa + LPS | 28.9 |
| Monocytes rest | 59.9 | Neutrophils rest | 13.9 |
| Monocytes LPS | 55.5 | Colon | 21.6 |
| Macrophages rest | 49.0 | Lung | 30.1 |
| Macrophages LPS | 28.9 | Thymus | 92.0 |
| HUVEC none | 16.2 | Kidney | 74.7 |
| HUVEC starved | 27.9 | | |

TABLE DK
Panel 4D

| | Rel. Exp. (%) | | Rel. Exp. (%) |
|---|---|---|---|
| | Ag531, Run | | Ag531, Run |
| Tissue Name | 165919167 | Tissue Name | 165919167 |
| Secondary Th1 act | 18.4 | HUVEC IL-1beta | 2.1 |
| Secondary Th2 act | 0.0 | HUVEC IFN gamma | 5.4 |
| Secondary Tr1 act | 16.8 | HUVEC TNF alpha + IFN gamma | 6.1 |
| Secondary Th1 rest | 4.4 | HUVEC TNF alpha + IL4 | 6.6 |
| Secondary Th2 rest | 7.8 | HUVEC IL-11 | 3.4 |
| Secondary Tr1 rest | 7.3 | Lung Microvascular EC none | 5.8 |
| Primary Th1 act | 5.1 | Lung Microvascular EC TNFalpha + | 7.0 |
| | | IL-1beta | |
| Primary Th2 act | 16.6 | Microvascular Dermal EC none | 7.2 |
| Primary Tr1 act | 18.3 | Microsvasular Dermal EC | 5.8 |
| | | TNFalpha + IL-1beta | |
| Primary Th1 rest | 31.9 | Bronchial epithelium TNFalpha + | 8.4 |
| | | IL1beta | |
| Primary Th2 rest | 16.2 | Small airway epithelium none | 2.2 |
| Primary Tr1 rest | 8.4 | Small airway epithelium TNFalpha + | 14.4 |
| | | IL-1beta | |
| CD45RA CD4 lymphocyte | 10.2 | Coronery artery SMC rest | 3.9 |
| act | | | |
| CD45RO CD4 lymphocyte | 17.4 | Coronery artery SMC TNFalpha + | 3.9 |
| act | | IL-1beta | |
| CD8 lymphocyte act | 13.7 | Astrocytes rest | 3.7 |
| Secondary CD8 lymphocyte | 15.6 | Astrocytcs TNFalpha + IL-1beta | 3.3 |
| rest | | | |
| Secondary CD8 lymphocyte | 10.5 | KU-812 (Basophil) rest | 10.3 |
| act | | | |
| CD4 lymphocyte none | 4.2 | KU-812 (Basophil) PMA/ionomycin | 31.0 |
| 2ry Th1/Th2/Tr1_anti-CD95 | 10.3 | CCD1106 (Keratinocytcs) none | 7.3 |
| CH11 | | | |
| LAK cells rest | 15.0 | CCD1106 (Keratinocytes) | 1.8 |
| | | TNFalpha + IL-1beta | |
| LAK cells IL-2 | 14.9 | Liver cirrhosis | 2.2 |
| LAK cells IL-2 + IL-12 | 10.1 | Lupus kidney | 1.8 |
| LAK cells IL-2 + IFN gamma | 15.7 | NCI-H292 none | 12.5 |
| LAK cells IL-2 + IL-18 | 14.8 | NCI-H292 IL-4 | 17.1 |
| LAK cells PMA/ionomycin | 11.7 | NCI-H292 IL-9 | 8.5 |
| NK Cells IL-2 rest | 13.2 | NCI-H292 IL-13 | 15.5 |
| Two Way MLR 3 day | 18.3 | NCI-H292 IFN gamma | 12.9 |
| Two Way MLR 5 day | 8.0 | HPAEC none | 5.7 |
| Two Way MLR 7 day | 7.1 | HPAEC TNF alpha + IL-1 beta | 7.3 |
| PBMC rest | 4.9 | Lung fibroblast none | 8.4 |
| PBMC PWM | 61.6 | Lung fibroblast TNF alpha + IL-1 | 4.0 |
| | | beta | |
| PBMC PHA-L | 19.8 | Lung fibroblast IL-4 | 13.6 |
| Ramos (B cell) none | 19.1 | Lung fibroblast IL-9 | 9.7 |
| Ramos (B cell) ionomycin | 100.0 | Lung fibroblast IL-13 | 7.9 |
| B lymphocytes PWM | 36.1 | Lung fibroblast IFN gamma | 11.7 |
| B lymphocytes CD40L and | 25.0 | Dermal fibroblast CCD1070 rest | 14.4 |
| IL-4 | | | |
| EOL-1 dbcAMP | 13.0 | Dermal fibroblast CCD1070 TNF | 50.0 |
| | | alpha | |
| EOL-1 dbcAMP | 13.4 | Dermal fibroblast CCD1070 IL-1 | 10.8 |
| PMA/ionomycin | | beta | |
| Dendritic cells none | 7.0 | Dermal fibroblast IFN gamma | 10.1 |
| Dendritic cells LPS | 8.3 | Dermal fibroblast IL-4 | 22.1 |
| Dendritic cells anti-CD40 | 8.7 | IBD Colitis 2 | 1.4 |
| Monocytes rest | 7.3 | IBD Crohn's | 1.5 |
| Monocytes LPS | 6.7 | Colon | 11.8 |
| Macrophagcs rest | 9.6 | Lung | 6.8 |
| Macrophages LPS | 4.2 | Thymus | 33.0 |
| HUVEC none | 3.9 | Kidney | 22.7 |
| HUVEC starved | 8.0 | | |

CNS_neurodegeneration_v1.0 Summary: Ag3690 This panel confirms the expression of the CG57547-01 gene at low levels in the brain in an independent group of individuals. Interestingly, this gene appears to be upregulated in the temporal cortex of Alzheimer's disease patients. Therefore, blockade of the transient receptor potential-related protein encoded for by this gene may be of use in the treatment of this disease and decrease neuronal death. Ag531 Expression of this gene is low/undetectable (CTs>35) across all of the samples on this panel (data not shown). It is likely that there were experimental difficulties with this run.

General_screening_panel_v1.4 Summary: Ag3690 The CG57547-01 gene is expressed at moderate levels across all of the samples on this panel with highest expression detected in breast cancer cell line MCF-7 (CT=27.5). In general, the pattern of gene expression is consistent with that observed in Panels 1.1 and 1.2. Please see Panel 1.1 for a discussion of the potential relevance of this expression pattern in CNS and metabolically relevant tissues.

In addition, consistent with what is seen in Panel 1.2, this gene is expressed at higher levels in fetal liver (CT=29.5) than in adult liver (CT=33.6). Therefore, expression of this gene may be used to distinguish fetal liver from adult liver.

Panel 1.1 Summary: Ag534 The CG57547-01 gene encodes a protein with homology to melastatin, a member of the transient receptor potential (Trp) family of calcium ion channels. The CG57547-01 gene is expressed at moderate to high levels across all the samples on this panel with the highest expression detected in kidney (CT=25). Interestingly, defects in ion channels are associated with kidney disorders, such as Bartter's syndrome, policystic kidney disease and Dent's disease (ref. 1), suggesting that the CG57547-01 gene may also play a role in kidney homeostasis.

Furthermore, this gene is expressed in a variety of metabolically relevant tissues, including adrenal gland, heart, skeletal muscle, liver, pancreas, pituitary gland, and thyroid. Therefore, as a classical drug target, the CG57547-01 protein may be useful for the treatment of disease in any or all of these tissues, including diabetes and obesity. In support of this hypothesis, mutations in ion channels have previously been associated with hyperinsulinemic hypoglycemia of infancy (ref. 1).

Among CNS samples, the CG57547-01 gene is expressed in hippocampus, substantia nigra, thalamus, and cerebral cortex with highest expression detected in the cerebellum (CT values<30). The protein encoded by the CG57547-01 gene shows considerable homology to known ion channels, which are the primary targets of all known antiepileptics. Furthermore, all gene mutations known to cause epilepsy or seizure disorders are found in ion channels (ref. 1-2). Two established antiepileptics (valproate and carbamazepine) also have efficacy in the treatment of bipolar disorder. Therefore, therapeutic modulation of this gene or its protein product may be beneficial in the treatment of these disorders as may agonism/antagonism of the ion channel.

Interestingly, it also appears that there is a difference in CG57547-01 gene expression between several adult tissues and their fetal counterparts. Specifically, expression of this gene is significantly higher in adult kidney, liver, skeletal muscle and heart when compared to the corresponding fetal tissues. Thus, the expression of the CG57547-01 gene could be used as a marker of adult tissues, or alternatively its relative absence could be used as a marker of fetal tissues. Since fetal tissues show potential use for organ regeneration, the expression of this gene may be inhibitory to organogenesis. Thus, the therapeutic modulation of the activity of the CG57547-01 gene product, through the use of small molecule drugs or antibodies, might be of use for the treatment of diseases whose pathology is characterized by organ degeneration.

References:

1. Dworakowska B., Dolowy K. (2000) Ion channels-related diseases. Acta Biochim. Pol. 47: 685-703.

There are many diseases related to ion channels. Mutations in muscle voltage-gated sodium, potassium, calcium and chloride channels, and acetylcholine-gated channel may lead to such physiological disorders as hyper- and hypokalemic periodic paralysis, myotonias, long QT syndrome, Brugada syndrome, malignant hyperthermia and myasthenia. Neuronal disorders, e.g., epilepsy, episodic ataxia, familial hemiplegic migraine, Lambert-Eaton myasthenic syndrome, Alzheimer's disease, Parkinson's disease, schizophrenia, hyperekplexia may result from dysfunction of voltage-gated sodium, potassium and calcium channels, or acetylcholine- and glycine-gated channels. Some kidney disorders, e.g., Bartter's syndrome, policystic kidney disease and Dent's disease, secretion disorders, e.g., hyperinsulinemic hypoglycemia of infancy and cystic fibrosis, vision disorders, e.g., congenital stationary night blindness and total colour-blindniess may also be linked to mutations in ion channels.

PMID: 11310970

2. Li M., Lester H. A. (2001) Ion channel diseases of the central nervous system. CNS Drug Rev. 7: 214-240.

In the last decade, advances in molecular genetics and cellular electrophysiology have increased our understanding of ion channel function. A number of diseases termed “channelopathies” have been discovered that are caused by ion channel dysfunction. Channelopathies can be caused by autoimmune, iatrogenic, toxic or genetic mechanisms. Mutations in genes encoding ion channel proteins that disrupt channel function are now the most commonly identified cause of channelopathics, perhaps because gene disruption is readily detected by the methods of molecular genetics. Ion channels are abundant in the central nervous system (CNS), but CNS channelopathies are rare; however, they overlap with some important neurological disorders, such as epilepsy, ataxia, migraine, schizophrenia, Alzheimer's disease and other neurodegenerative diseases. It is possible that more CNS channelopathies will be discovered when additional ion channels are characterized and the complex mechanisms of brain function are better understood. At present, increased knowledge of the identity, structure and function of ion channels is facilitating diagnosis and treatment of many channelopathies.

PMID: 11474425

Panel 1.2 Summary: Ag535 The CG57547-01 gene is expressed at moderate to high levels across all the samples on this panel with the highest expression detected in lung cancer cell line NCI-H1522 (CT=25). In general, the pattern of CG57547-01 gene expression is consistent with that observed in Panel 1.1; see Panel 1.1 for discussion of expression pattern in CNS and metabolically relevant tissues.

Interestingly, there appears to be a difference in CG57547-01 gene expression between fetal and adult liver. In addition, this gene is also highly expressed in pancreas (CT=25). Thus, the relative expression of the CG57547-01 gene might be useful as a marker of pancreas tissue. Furthermore, since this gene appears to be differentially expressed in adult and fetal liver, and that fetal liver represents a state of organogenesis, the therapeutic down-modulation of this gene product, through the use of small molecule drugs or antibodies might be of use in the treatment of diseases involving liver degeneration.

Panel 1.3D Summary: Ag531 This probe/primer set produced a similar pattern of expression as is seen in Panels 1.1, 1.2, and 1.4 using different probe/primer sets, although the expression level is lower in this experiment. This gene is expressed at highest levels in the breast cancer cell line MCF-7 (CT=33) and is also expressed at low levels in a number of other samples on this panel. Please see Panel 1.1 for discussion of expression pattern in CNS and metabolically relevant tissues.

Panel 4.1D Summary: Ag3690 The CG57547-01 gene is expressed at low to moderate levels in each of the cells and tissues examined on this panel. This observation suggests that this gene plays an important role in a variety of immunologically relevant cell types. Interestingly, calcium release activated calcium channels have been shown to be required for T cell activation, cytokine synthesis, and proliferation (ref. 1).

References:

1. Lepple-Wienhues A., Belka C., Laun T., Jekle A., Walter B., Wieland U., Welz M., Heil L., Kun J., Busch G., Weller M., Bamberg M., GLulbins E., Lang F. (1999) Stimulation of CD95 (Fas) blocks T lymphocyte calcium channels through sphingomyelinase and sphingolipids. Proc. Natl. Acad. Sci. USA 96: 13795-13800.

Calcium influx through store-operated calcium release-activated calcium channels (CRAC) is required for T cell activation, cytokine synthesis, and proliferation. The CD95 (Apo-1/Fas) receptor plays a role in self-tolerance and tumor immune escape, and it mediates apoptosis in activated T cells. CD95-stimulation blocks CRAC and Ca(2+) influx in lymphocytes through the activation of acidic sphingomyelinase (ASM) and ceramide release. The block of Ca(2+) entry is lacking in CD95-defective lpr lymphocytes as well as in ASM-defective cells and can be restored by retransfection of ASM. C2 ceramide, C6 ceramide, and sphingosinie block CRAC reversibly, whereas the inactive dihydroceramidc has no effect. CD95-stimulation or the addition of ceramide prevents store-operated Ca(2+) influx, activation of the transcriptional regulator NFAT, and IL-2 synthesis. The block of CRAC by sphingomyelinase metabolites adds a function to the repertoire of the CD95 receptor inhibiting T cell activation signals.

PMID: 10570152

Panel 4D Summary: Ag531 This probe/primer set produced a similar pattern of expression as was seen in Panel 4.1D using probe/primer set Ag3690, although the levels of expression are lower on Panel 4D. Expression of the CG57547-01 gene is highest in Ramos B cells treated with ionomycin (CT=30.4). Moderate expression of this gene is also seen in other B cell samples including peripheral blood mononuclear cells treated with pokeweed mitogen and B lymphocytes. Expression of this transcript in B cells suggests that this gene may be involved in rheumatic disease including rheumatoid arthritis, lupus, osteoarthritis, and hyperproliferative B cell disorders. Low but significant expression of this gene is seen in the other samples on this panel.

NOV6

Expression of gene NOV6/CG57611′-01 was assessed using the primer-probe set Ag3295, described in Table EA. Results of the RTQ-PCR runs are shown in Tables EB, EC and ED.

TABLE EA
Probe Name Ag3295

| Primers | Sequences | Length | Start Position | SEQ ID NO |
|---|---|---|---|---|
| Forward | 5′-caagccttccttcactgttaag-3′ | 22 | 396 | 267 |
| Probe | TET-5′-ccccagcttcaaacatttctactcaa-3′-TAMRA | 26 | 419 | 268 |
| Reverse | 5′-gcctcaacagacagtttggtat-3′ | 22 | 452 | 269 |

TABLE EB
CNS_neurodegeneration_v1.0

| | Rel. Exp. (%) | | Rel. Exp. (%) |
|---|---|---|---|
| | Ag3295, Run | | Ag3295, Run |
| Tissue Name | 210062303 | Tissue Name | 210062303 |
| AD 1 Hippo | 9.7 | Control (Path) 3 Temporal | 17.0 |
| | | Ctx | |
| AD 2 Hippo | 29.1 | Control (Path) 4 Temporal | 40.6 |
| | | Ctx | |
| AD 3 Hippo | 28.3 | AD 1 Occipital Ctx | 32.8 |
| AD 4 Hippo | 8.1 | AD 2 Occipital Ctx | 0.0 |
| | | (Missing) | |
| AD 5 hippo | 17.4 | AD 3 Occipital Ctx | 29.3 |
| AD 6 Hippo | 64.6 | AD 4 Occipital Ctx | 15.0 |
| Control 2 Hippo | 63.7 | AD 5 Occipital Ctx | 66.0 |
| Control 4 Hippo | 16.6 | AD 6 Occipital Ctx | 73.2 |
| Control (Path) 3 Hippo | 0.0 | Control 1 Occipital Ctx | 8.5 |
| AD 1 Temporal Ctx | 41.2 | Control 2 Occipital Ctx | 24.7 |
| AD 2 Temporal Ctx | 35.4 | Control 3 Occipital Ctx | 43.2 |
| AD 3 Temporal Ctx | 48.3 | Control 4 Occipital Ctx | 10.4 |
| AD 4 Temporal Ctx | 36.9 | Control (Path) 1 Occipital | 95.3 |
| | | Ctx | |
| AD 5 Inf Temporal Ctx | 74.7 | Control (Path) 2 Occipital | 7.2 |
| | | Ctx | |
| AD 5 Sup Temporal Ctx | 65.5 | Control (Path) 3 Occipital | 1.4 |
| | | Ctx | |
| AD 6 Inf Temporal Ctx | 85.9 | Control (Path) 4 Occipital | 25.7 |
| | | Ctx | |
| AD 6 Sup Temporal Ctx | 49.0 | Control 1 Parietal Ctx | 10.6 |
| Control 1 Temporal Ctx | 8.4 | Control 2 Parietal Ctx | 100.0 |
| Control 2 Temporal Ctx | 42.9 | Control 3 Parietal Ctx | 14.1 |
| Control 3 Temporal Ctx | 23.0 | Control (Path) 1 Parietal | 47.0 |
| | | Ctx | |
| Control 4 Temporal Ctx | 31.0 | Control (Path) 2 Parietal | 12.4 |
| | | Ctx | |
| Control (Path) 1 Temporal | 62.4 | Control (Path) 3 Parietal | 4.8 |
| Ctx | | Ctx | |
| Control (Path) 2 Temporal | 42.6 | Control (Path) 4 Parietal | 45.7 |
| Ctx | | Ctx | |

TABLE EC
General_screening_panel_v1.4

| | Rel. Exp. (%) | | Rel. Exp. (%) |
|---|---|---|---|
| | Ag3295, Run | | Ag3295, Run |
| Tissue Name | 215669651 | Tissue Name | 215669651 |
| Adipose | 0.2 | Renal ca. TK-10 | 18.2 |
| Melanoma* Hs688(A).T | 0.1 | Bladder | 0.7 |
| Melanoma* Hs688(B).T | 0.4 | Gastric ca. (liver met.) NCI- | 4.3 |
| | | N87 | |
| Melanoma* M14 | 2.6 | Gastric ca. KATO III | 2.3 |
| Melanoma* LOXIMVI | 0.1 | Colon ca. SW-948 | 0.2 |
| Melanoma* SK-MEL-5 | 1.3 | Colon ca. SW480 | 3.2 |
| Squamous cell carcinoma | 0.5 | Colon ca.* (SW480 met) | 0.8 |
| SCC-4 | | SW620 | |
| Testis Pool | 0.5 | Colon ca. HT29 | 29.1 |
| Prostate ca.* (bone met) | 0.9 | Colon ca. HCT-116 | 1.3 |
| PC-3 | | | |
| Prostate Pool | 3.6 | Colon ca. CaCo-2 | 4.9 |
| Placenta | 1.0 | Colon cancer tissue | 1.3 |
| Uterus Pool | 1.4 | Colon ca. SW1116 | 0.7 |
| Ovarian ca. OVCAR-3 | 0.2 | Colon ca. Colo-205 | 0.6 |
| Ovarian ca. SK-OV-3 | 0.9 | Colon ca. SW-48 | 0.4 |
| Ovarian ca. OVCAR-4 | 0.3 | Colon Pool | 0.9 |
| Ovarian ca. OVCAR-5 | 4.2 | Small Intestine Pool | 1.2 |
| Ovarian ca. IGROV-1 | 0.4 | Stomach Pool | 0.1 |
| Ovarian ca. OVCAR-8 | 0.9 | Bone Marrow Pool | 2.9 |
| Ovary | 0.2 | Fetal Heart | 0.1 |
| Breast ca. MCF-7 | 0.4 | Heart Pool | 0.0 |
| Breast ca. MDA-MB-231 | 0.6 | Lymph Node Pool | 0.1 |
| Breast ca. BT 549 | 2.9 | Fetal Skeletal Muscle | 0.1 |
| Breast ca. T47D | 20.4 | Skeletal Muscle Pool | 0.5 |
| Breast ca. MDA-N | 2.0 | Spleen Pool | 0.5 |
| Breast Pool | 0.4 | Thymus Pool | 0.5 |
| Trachea | 12.5 | CNS cancer (glio/astro) U87- | 3.4 |
| | | MG | |
| Lung | 0.3 | CNS cancer (glio/astro) U- | 0.4 |
| | | 118-MG | |
| Fetal Lung | 0.5 | CNS cancer (neuro; met) SK- | 0.6 |
| | | N-AS | |
| Lung ca. NCI-N417 | 0.0 | CNS cancer (astro) SF-539 | 0.1 |
| Lung ca. LX-I | 2.0 | CNS cancer (astro) SNB-75 | 2.2 |
| Lung ca. NCI-H146 | 0.6 | CNS cancer (glio) SNB-19 | 1.3 |
| Lung ca. SHP-77 | 1.4 | CNS cancer (glio) SF-295 | 3.7 |
| Lung ca. A549 | 27.4 | Brain (Amygdala) Pool | 0.8 |
| Lung ca. NCI-H526 | 0.2 | Brain (cerebellum) | 3.3 |
| Lung ca. NCI-H23 | 3.1 | Brain (fetal) | 1.5 |
| Lung ca. NCI-H460 | 2.7 | Brain (Hippocampus) Pool | 0.5 |
| Lung ca. HOP-62 | 0.2 | Cerebral Cortex Pool | 0.6 |
| Lung ca. NCI-H522 | 4.3 | Brain (Substantia nigra) Pool | 0.4 |
| Liver | 0.4 | Brain (Thalamus) Pool | 0.8 |
| Fetal Liver | 1.1 | Brain (whole) | 0.5 |
| Liver ca. HepG2 | 42.9 | Spinal Cord Pool | 1.1 |
| Kidney Pool | 1.0 | Adrenal Gland | 0.2 |
| Fetal Kidney | 0.5 | Pituitary gland Pool | 0.1 |
| Renal ca. 786-0 | 0.3 | Salivary Gland | 100.0 |
| Renal ca. A498 | 5.1 | Thyroid (female) | 0.1 |
| Renal ca. ACHN | 0.4 | Pancreatic ca. CAPAN2 | 2.8 |
| Renal ca. UO-31 | 0.3 | Pancreas Pool | 0.7 |

TABLE ED
Panel 4D

| | Rel. Exp. (%) | | Rel. Exp. (%) |
|---|---|---|---|
| | Ag3295, Run | | Ag3295, Run |
| Tissue Name | 164331710 | Tissue Name | 164331710 |
| Secondary Th1 act | 0.8 | HUVEC IL-1beta | 0.0 |
| Secondary Th2 act | 23.8 | HUVEC IFN gamma | 3.2 |
| Secondary Tr1 act | 11.9 | HUVEC TNF alpha + IFN gamma | 3.0 |
| Secondary Th1 rest | 0.0 | HUVEC TNF alpha + IL4 | 0.0 |
| Secondary Th2 rest | 2.4 | HUVEC IL-11 | 0.0 |
| Secondary Tr1 rest | 3.0 | Lung Microvascular EC none | 1.8 |
| Primary Th1 act | 12.5 | Lung Microvascular EC TNFalpha + | 3.7 |
| | | IL-1beta | |
| Primary Th2 act | 47.0 | Microvascular Dermal EC none | 4.0 |
| Primary Tr1 act | 30.1 | Microsvasular Dermal EC | 0.0 |
| | | TNFalpha + IL-1beta | |
| Primary Th1 rest | 0.0 | Bronchial epithelium TNFalpha + | 9.5 |
| | | IL1beta | |
| Primary Th2 rest | 1.4 | Small airway epithelium none | 100.0 |
| Primary Tr1 rest | 11.5 | Small airway epithelium TNFalpha + | 55.5 |
| | | IL-1beta | |
| CD45RA CD4 lymphocyte | 0.7 | Coronery artery SMC rest | 0.0 |
| act | | | |
| CD45RO CD4 lymphocyte | 0.0 | Coronery artery SMC TNFalpha + | 0.0 |
| act | | IL-1beta | |
| CD8 lymphocyte act | 5.0 | Astrocytes rest | 1.3 |
| Secondary CD8 lymphocyte | 5.9 | Astrocytcs TNFalpha + IL-1beta | 0.0 |
| rest | | | |
| Secondary CD8 lymphocyte | 8.8 | KU-812 (Basophil) rest | 2.5 |
| act | | | |
| CD4 lymphocyte none | 0.0 | KU-812 (Basophil) | 3.9 |
| | | PMA/ionomycin | |
| 2ry Th1/Th2/Tr1_anti-CD95 | 3.6 | CCD1106 (Keratinocytcs) none | 2.7 |
| CH11 | | | |
| LAK cells rest | 17.7 | CCD1106 (Keratinocytes) | 14.9 |
| | | TNFalpha + IL-1beta | |
| LAK cells IL-2 | 6.2 | Liver cirrhosis | 11.1 |
| LAK cells IL-2 + IL-12 | 1.6 | Lupus kidney | 0.0 |
| LAK cells IL-2 + IFN gamma | 5.7 | NCI-H292 none | 1.4 |
| LAK cells IL-2 + IL-18 | 9.9 | NCI-H292 IL-4 | 3.1 |
| LAK cells PMA/ionomycin | 8.0 | NCI-H292 IL-9 | 7.1 |
| NK Cells IL-2 rest | 3.3 | NCI-H292 IL-13 | 1.0 |
| Two Way MLR 3 day | 9.9 | NCI-H292 IFN gamma | 1.6 |
| Two Way MLR 5 day | 8.7 | HPAEC none | 1.2 |
| Two Way MLR 7 day | 3.2 | HPAEC TNF alpha + IL-1beta | 0.0 |
| PBMC rest | 0.0 | Lung fibroblast none | 3.4 |
| PBMC PWM | 21.0 | Lung fibroblast TNF alpha + IL-1 | 0.0 |
| | | beta | |
| PBMC PHA-L | 14.9 | Lung fibroblast IL-4 | 0.5 |
| Ramos (B cell) none | 2.2 | Lung fibroblast IL-9 | 3.7 |
| Ramos (B cell) ionomycin | 7.0 | Lung fibroblast IL-13 | 1.5 |
| B lymphocytes PWM | 18.6 | Lung fibroblast IFN gamma | 1.6 |
| B lymphocytes CD40L and | 7.5 | Dermal fibroblast CCD1070 rest | 14.0 |
| IL-4 | | | |
| EOL-1 dbcAMP | 0.0 | Dermal fibroblast CCD1070 TNF | 14.7 |
| | | alpha | |
| EOL-I dbcAMP | 1.5 | Dermal fibroblast CCD1070 IL-1 | 2.7 |
| PMA/ionomycin | | beta | |
| Dendritic cells none | 6.4 | Dermal fibroblast IFN gamma | 3.5 |
| Dendritic cells LPS | 11.0 | Dermal fibroblast IL-4 | 6.6 |
| Dendritic cells anti-CD40 | 8.4 | IBD Colitis 2 | 0.0 |
| Monocytes rest | 0.0 | IBD Crohn's | 0.0 |
| Monocytes LPS | 6.7 | Colon | 41.5 |
| Macrophages rest | 4.9 | Lung | 16.7 |
| Macrophagcs LPS | 8.1 | Thymus | 0.0 |
| HUVEC none | 0.0 | Kidney | 13.9 |
| HUVEC starved | 1.8 | | |

CNS_neurodegeneration_v1.0 Summary: Ag3295 This panel confirms the expression of this gene at low levels in the brain in an independent group of individuals (CTs=33-35). However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Af3295 Expression of the CG57611-01 gene is highest in the salivary gland (CT=28). Therefore, expression of this gene may be used to distinguish salivary gland from the other samples on this panel. In general, expression of this gene appears to be higher in several cancer cell lines, including lung, breast, colon and liver cancer, when compared to normal tissues. Thus, therapeutic modulation of the activity of this gene or its protein product, using small molecule drugs, antibodies or protein therapeutics, may be of benefit in the treatment of lung, breast, colon and liver cancer. This gene encodes a protein with homology to CD22, a B lymphocyte-restricted adhesion molecule. Antibodies to the CD22 protein have been used as a therapeutic treatment for lymphomas (ref. 1), suggesting that the CG57611-01 protein may also be an attractive target for the treatment of leukemias and lymphomas.

This gene is also expressed at low but significant levels in the spinal cord, cerebellum, and amygdala. Therefore, this gene may play a role in central nervous system disorders such as Alzheimer's disease, Parkinson's disease, epilepsy, multiple sclerosis, schizophrenia and depression.

References:

1. Rybak S M, Newton D L. Antibody targeted therapeutics for lymphoma: new focus on the CD22 antigen and RNA. Expert Opin Biol Ther 2001 November;1(6):995-1003

The approval of antibodies for cancer treatment has provoked increased interest in the development of new and improved antibody-mediated therapies. This emerging approach centres on targeting CD22 on human B-cells with a monoclonal antibody (mAb). Anti-CD22 antibodies conjugated to a cytotoxic RNAse elicits potent and specific killing of the lymphoma cells in vitro and in human lymphoma models in severe combined immune deficiency (SCID) mice. RNA damage caused by RNAses could be an important alternative to standard DNA damaging chemotherapeutics. Moreover, targeted RNAses may overcome problems of toxicity and immunogenicity associated with plant- or bacterial toxin-containing immunotoxins.

Panel 4D Summary: Ag3295 Expression of this gene is highest in small airway epithelium, irrespective of treatment (CTs=31-32). Therefore, modulation of the expression or activity of the protein encoded by this transcript through the application of small molecule therapeutics may be useful in the treatment of asthma, COPD, and emphysema. Low but significant expression of this gene is seen in a number of samples on this panel. Expression of this gene appears to be higher in activated T cells than in resting T cells, suggesting that expression of this gene could be used to distinguish these two types of T cells.

NOV8

Expression of gene NOV8a/CG57452-01 and NOV8b/CG57452-02 was assessed using the primer-probe sets Ag3243 and Ag878, described in Tables FA and FB. Results of the RTQ-PCR runs are shown in Tables FC, FD, FE, FF, FG and FH. Please note that the CG57452-02 is only recognized by the Ag878 primer-probe set.

Table FA. Probe Name Ag3243

TABLE FA
Probe Name Ag3243

| Primers | Sequences | Length | Start Position | SEQ ID NO |
|---|---|---|---|---|
| Forward | 5′-ccatttcggacagtctcaatt-3′ | 121 | 1313 | 270 |
| Probe | TET-5′-cctcacctttaagaatagtagctctggaca-3′-TAMRA | 30 | 1337 | 271 |
| Reverse | 5′-aggtgaagctctgggtctttt-3′ | 21 | 1383 | 272 |

TABLE FB
Probe Name Ag878

| Primers | Sequences | Length | Start Position | SEQ ID NO |
|---|---|---|---|---|
| Forward | 5′-agccctgttgttttattgatga-3′ | 22 | 1745 | 273 |
| Probe | TET-5′-cctgagccccaacaaggatttcatat-3′-TAMRA | 26 | 1710 | 274 |
| Reverse | 5′-cagacgaagggtcaaatgg-3′ | 19 | 1682 | 275 |

TABLE FC
CNS_neurodegeneration_v1.0

| | Rel. Exp. (%) | | Rel. Exp. (%) |
|---|---|---|---|
| | Ag3243, Run | | Ag3243, Run |
| Tissue Name | 210037850 | Tissue Name | 210037850 |
| AD 1 Hippo | 33.2 | Control (Path) 3 Temporal | 18.7 |
| | | Ctx | |
| AD 2 Hippo | 49.7 | Control (Path) 4 Temporal | 74.7 |
| | | Ctx | |
| AD 3 Hippo | 30.8 | AD 1 Occipital Ctx | 33.9 |
| AD 4 Hippo | 25.3 | AD 2 Occipital Ctx | 0.0 |
| | | (Missing) | |
| AD 5 hippo | 52.1 | AD 3 Occipital Ctx | 8.2 |
| AD 6 Hippo | 49.7 | AD 4 Occipital Ctx | 35.6 |
| Control 2 Hippo | 97.3 | AD 5 Occipital Ctx | 14.0 |
| Control 4 Hippo | 31.9 | AD 6 Occipital Ctx | 26.4 |
| Control (Path) 3 Hippo | 30.4 | Control 1 Occipital Ctx | 4.9 |
| AD 1 Temporal Ctx | 48.0 | Control 2 Occipital Ctx | 35.6 |
| AD 2 Temporal Ctx | 38.4 | Control 3 Occipital Ctx | 24.8 |
| AD 3 Temporal Ctx | 16.6 | Control 4 Occipital Ctx | 17.0 |
| AD 4 Temporal Ctx | 51.4 | Control (Path) 1 Occipital | 100.0 |
| | | Ctx | |
| AD 5 Inf Temporal Ctx | 51.4 | Control (Path) 2 Occipital | 35.6 |
| | | Ctx | |
| AD 5 Sup Temporal Ctx | 80.1 | Control (Path) 3 Occipital | 6.1 |
| | | Ctx | |
| AD 6 Inf Temporal Ctx | 32.8 | Control (Path) 4 Occipital | 43.8 |
| | | Ctx | |
| AD 6 Sup Temporal Ctx | 32.5 | Control 1 Parietal Ctx | 19.8 |
| Control 1 Temporal Ctx | 43.8 | Control 2 Parietal Ctx | 52.1 |
| Control 2 Temporal Ctx | 26.1 | Control 3 Parietal Ctx | 9.7 |
| Control 3 Temporal Ctx | 42.3 | Control (Path) 1 Parietal | 62.4 |
| | | Ctx | |
| Control 4 Temporal Ctx | 26.1 | Control (Path) 2 Parietal | 50.3 |
| | | Ctx | |
| Control (Path) 1 Temporal | 93.3 | Control (Path) 3 Parietal | 10.9 |
| Ctx | | Ctx | |
| Control (Path) 2 Temporal | 77.9 | Control (Path) 4 Parietal | 46.7 |
| Ctx | | Ctx | |

TABLE FD
General_screening_panel_v1.4

| | Rel. Exp. (%) | | Rel. Exp. (%) |
|---|---|---|---|
| | Ag3243, | | Ag3243, |
| Tissue Name | Run 214693632 | Tissue Name | Run 214693632 |
| Adipose | 0.0 | Renal ca. TK-10 | 0.0 |
| Melanoma* Hs688(A).T | 0.0 | Bladder | 0.9 |
| Melanoma* Hs688(B).T | 0.0 | Gastric ca. (liver met.) NCI- | 0.0 |
| | | N87 | |
| Melanoma* M14 | 0.0 | Gastric ca. KATO III | 0.0 |
| Melanoma* LOXIMVI | 1.7 | Colon ca. SW-948 | 0.0 |
| Melanoma* SK-MEL-5 | 0.5 | Colon ca. SW480 | 0.7 |
| Squamous cell carcinoma | 0.0 | Colon ca.* (SW480 met) | 0.0 |
| SCC-4 | | SW620 | |
| Testis Pool | 3.4 | Colon ca. HT29 | 0.0 |
| Prostate ca.* (bone met) | 0.7 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 0.2 |
| Uterus Pool | 0.0 | Colon ca. SW1116 | 0.0 |
| Ovarian ca. OVCAR-3 | 0.8 | Colon ca. Colo-205 | 0.0 |
| Ovarian ca. SK-OV-3 | 8.1 | Colon ca. SW-48 | 0.0 |
| Ovarian ca. OVCAR-4 | 0.0 | Colon Pool | 0.2 |
| Ovarian ca. OVCAR-5 | 0.0 | Small Intestine Pool | 5.3 |
| Ovarian ca. IGROV-1 | 0.0 | Stomach Pool | 0.3 |
| Ovarian ca. OVCAR-8 | 2.8 | Bone Marrow Pool | 0.0 |
| Ovary | 0.5 | Fetal Heart | 1.5 |
| Breast ca. MCF-7 | 0.0 | Heart Pool | 0.3 |
| Breast ca. MDA-MB-231 | 0.0 | Lymph Node Pool | 0.0 |
| Breast ca. BT 549 | 0.2 | Fetal Skeletal Muscle | 1.4 |
| Breast ca. T47D | 0.4 | Skeletal Muscle Pool | 0.0 |
| Breast ca. MDA-N | 0.0 | Spleen Pool | 9.4 |
| Breast Pool | 0.8 | Thymus Pool | 2.9 |
| Trachea | 0.3 | CNS cancer (glio/astro) U87-MG | 3.1 |
| Lung | 0.0 | CNS cancer (glio/astro) U-118-MG | 20.9 |
| Fetal Lung | 100.0 | CNS cancer (neuro; met) SK-N-AS | 0.7 |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 2.5 | CNS cancer (glio) SNB-19 | 0.0 |
| Lung ca. SHP-77 | 0.2 | CNS cancer (glio) SF-295 | 0.0 |
| Lung ca. A549 | 0.3 | Brain (Amygdala) Pool | 38.4 |
| Lung ca. NCI-H526 | 0.0 | Brain (cerebellum) | 5.1 |
| Lung ca. NCI-H23 | 1.4 | Brain (fetal) | 11.9 |
| Lung ca. NCI-H460 | 16.2 | Brain (Hippocampus) Pool | 42.9 |
| Lung ca. HOP-62 | 0.4 | Cerebral Cortex Pool | 44.8 |
| Lung ca. NCI-M522 | 1.1 | Brain (Substantia nigra) Pool | 28.5 |
| Liver | 0.0 | Brain (Thalamus) Pool | 54.3 |
| Fetal Liver | 0.0 | Brain (whole) | 32.8 |
| Liver ca. HepG2 | 0.0 | Spinal Cord Pool | 32.1 |
| Kidney Pool | 0.0 | Adrenal Gland | 26.2 |
| Fetal Kidney | 73.2 | Pituitary gland Pool | 17.9 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.5 | Pancreas Pool | 0.2 |

TABLE FE
Panel 1.2

| | Rel. Exp. (%) | | Rel. Exp. (%) |
|---|---|---|---|
| | Ag878, | | Ag878, |
| Tissue Name | Run 118826261 | Tissue Name | Run 118826261 |
| Endothelial cells | 4.0 | Renal ca. 786-0 | 0.0 |
| Heart (Fetal) | 2.8 | Renal ca. A498 | 0.3 |
| Pancreas | 6.5 | Renal ca. RXF 393 | 0.0 |
| Pancreatic ca. CAPAN 2 | 0.0 | Renal ca. ACHN | 0.1 |
| Adrenal Gland | 68.8 | Renal ca. UO-31 | 0.2 |
| Thyroid | 1.1 | Renal ca. TK-10 | 0.0 |
| Salivary gland | 5.7 | Liver | 4.6 |
| Pituitary gland | 100.0 | Liver (fetal) | 0.6 |
| Brain (fetal) | 73.7 | Liver ca. (hepatoblast) HepG2 | 0.0 |
| Brain (whole) | 53.2 | Lung | 27.2 |
| Brain (amygdala) | 46.7 | Lung (fetal) | 20.7 |
| Brain (cerebellum) | 20.2 | Lung ca. (small cell) LX-1 | 0.1 |
| Brain (hippocampus) | 46.3 | Lung ca. (small cell) NCI-H69 | 8.3 |
| Brain (thalamus) | 28.9 | Lung ca. (s. cell var.) SHP-77 | 0.0 |
| Cerebral Cortex | 45.7 | Lung ca. (large cell) NCI-H460 | 28.7 |
| Spinal cord | 27.4 | Lung ca. (non-sm. cell) A549 | 1.0 |
| glio/astro U87-MG | 4.3 | Lung ca. (non-s. cell) NCI-H23 | 0.8 |
| glio/astro U-118-MG | 8.3 | Lung ca. (non-s. cell) HOP-62 | 1.9 |
| astrocytoma SW1783 | 0.5 | Lung ca. (non-s. cl) NCI-H522 | 5.7 |
| neuro*; met SK-N-AS | 9.2 | Lung ca. (squam.) SW 900 | 0.1 |
| astrocytoma SF-539 | 0.7 | Lung ca. (squam.) NCI-H596 | 32.3 |
| astrocytoma SNB-75 | 0.0 | Mammary gland | 6.4 |
| glioma SNB-19 | 3.9 | Breast ca.* (pl. ef) MCF-7 | 0.0 |
| glioma U251 | 10.4 | Breast ca.* (pl. ef) MDA-MB-231 | 0.0 |
| glioma SF-295 | 2.4 | Breast ca.* (pl. ef) T47D | 5.3 |
| Heart | 16.2 | Breast ca. BT-549 | 1.2 |
| Skeletal Muscle | 3.9 | Breast ca. MDA-N | 0.4 |
| Bone marrow | 0.0 | Ovary | 7.7 |
| Thymus | 0.8 | Ovarian ca. OVCAR-3 | 12.2 |
| Spleen | 3.6 | Ovarian ca. OVCAR-4 | 0.0 |
| Lymph node | 24.0 | Ovarian ca. OVCAR-5 | 5.1 |
| Colorectal Tissue | 0.3 | Ovarian ca. OVCAR-8 | 1.9 |
| Stomach | 5.7 | Ovarian ca. IGROV-1 | 0.8 |
| Small intestine | 6.2 | Ovarian ca. (ascites) SK-OV-3 | 12.5 |
| Colon ca. SW480 | 0.3 | Uterus | 2.2 |
| Colon ca.* SW620 (SW480 | 0.0 | Placenta | 3.3 |
| met) | | | |
| Colon ca. HT29 | 0.3 | Prostate | 0.5 |
| Colon ca. HCT-116 | 0.0 | Prostate ca.* (bone met) PC-3 | 4.1 |
| Colon ca. CaCo-2 | 0.4 | Testis | 14.7 |
| Colon ca. Tissue | 2.6 | Melanoma Hs688(A).T | 0.1 |
| (ODO3866) | | | |
| Colon ca. HCC-2998 | 0.0 | Melanoma* (met) Hs688(B).T | 0.9 |
| Gastric ca.* (liver met) NCI- | 0.0 | Melanoma UACC-62 | 20.3 |
| N87 | | | |
| Bladder | 4.9 | Melanoma M14 | 1.4 |
| Trachea | 0.7 | Melanoma LOX IMVI | 5.6 |
| Kidney | 28.3 | Melanoma* (met) SK-MEL-5 | 0.2 |
| Kidney (fetal) | 33.7 | | |

TABLE FF
Panel 2.2

| | Rel. Exp. (%) | | Rel. Exp. (%) |
|---|---|---|---|
| | Ag3243, | | Ag3243, |
| Tissue Name | Run 174443356 | Tissue Name | Run 174443356 |
| Normal Colon | 0.0 | Kidney Margin (OD04348) | 20.2 |
| Colon cancer (OD06064) | 0.0 | Kidney malignant cancer | 0.0 |
| | | (OD06204B) | |
| Colon Margin (OD06064) | 0.0 | Kidney normal adjacent tissue | 5.2 |
| | | (OD06204E) | |
| Colon cancer (OD06159) | 0.0 | Kidney Cancer (OD04450-01) | 0.0 |
| Colon Margin (OD06159) | 0.0 | Kidney Margin (OD04450-03) | 34.2 |
| Colon cancer (OD06297-04) | 0.0 | Kidney Cancer 8120613 | 0.0 |
| Colon Margin (OD06297-05) | 0.0 | Kidney Margin 8120614 | 1.7 |
| CC Gr. 2 ascend colon | 0.0 | Kidney Cancer 9010320 | 0.0 |
| (ODO3921) | | | |
| CC Margin (ODO3921) | 0.0 | Kidney Margin 9010321 | 0.0 |
| Colon cancer metastasis | 0.0 | Kidney Cancer 8120607 | 0.0 |
| (OD06104) | | | |
| Lung Margin (OD06104) | 0.0 | Kidney Margin 8120608 | 3.4 |
| Colon mets to lung (OD04451- | 8.3 | Normal Uterus | 0.0 |
| 01) | | | |
| Lung Margin (OD04451-02) | 100.0 | Uterine Cancer 064011 | 0.0 |
| Normal Prostate | 0.0 | Normal Thyroid | 0.0 |
| Prostate Cancer (OD04410) | 0.0 | Thyroid Cancer 064010 | 0.0 |
| Prostate Margin (OD04410) | 0.0 | Thyroid Cancer A302152 | 3.4 |
| Normal Ovary | 0.0 | Thyroid Margin A302153 | 0.0 |
| Ovarian cancer (OD06283-03) | 0.0 | Normal Breast | 3.3 |
| Ovarian Margin (OD06283-07) | 0.0 | Breast Cancer (OD04566) | 0.0 |
| Ovarian Cancer 064008 | 0.0 | Breast Cancer 1024 | 0.0 |
| Ovarian cancer (OD06145) | 0.0 | Breast Cancer (OD04590-01) | 0.0 |
| Ovarian Margin (OD06145) | 0.0 | Breast Cancer Mets (OD04590- | 0.0 |
| | | 03) | |
| Ovarian cancer (OD06455-03) | 0.0 | Breast Cancer Metastasis | 15.1 |
| | | (OD04655-05) | |
| Ovarian Margin (OD06455-07) | 0.0 | Breast Cancer 064006 | 0.0 |
| Normal Lung | 30.4 | Breast Cancer 9100266 | 0.0 |
| Invasive poor diff. lung adeno | 0.0 | Breast Margin 9100265 | 0.0 |
| (ODO4945-01 | | | |
| Lung Margin (ODO4945-03) | 51.4 | Breast Cancer A209073 | 0.0 |
| Lung Malignant Cancer | 0.0 | Breast Margin A2090734 | 0.0 |
| (OD03126) | | | |
| Lung Margin (OD03126) | 9.9 | Breast cancer (OD06083) | 0.0 |
| Lung Cancer (OD05014A) | 6.9 | Breast cancer node metastasis | 2.9 |
| | | (OD06083) | |
| Lung Margin (OD05014B) | 21.2 | Normal Liver | 0.0 |
| Lung cancer (OD06081) | 0.0 | Liver Cancer 1026 | 0.0 |
| Lung Margin (OD06081) | 23.0 | Liver Cancer 1025 | 0.0 |
| Lung Cancer (OD04237-01) | 0.0 | Liver Cancer 6004-T | 0.0 |
| Lung Margin (OD04237-02) | 40.3 | Liver Tissue 6004-N | 0.0 |
| Ocular Melanoma Metastasis | 0.0 | Liver Cancer 6005-T | 0.0 |
| Ocular Melanoma Margin | 0.0 | Liver Tissue 6005-N | 0.0 |
| (Liver) | | | |
| Melanoma Metastasis | 0.0 | Liver Cancer 064003 | 3.0 |
| Melanoma Margin (Lung) | 52.5 | Normal Bladder | 0.0 |
| Normal Kidney | 16.3 | Bladder Cancer 1023 | 0.0 |
| Kidney Ca, Nuclear grade 2 | 28.7 | Bladder Cancer A302173 | 0.0 |
| (OD04338) | | | |
| Kidney Margin (OD04338) | 0.0 | Normal Stomach | 3.9 |
| Kidney Ca Nuclear grade 1/2 | 0.0 | Gastric Cancer 9060397 | 0.0 |
| (OD04339) | | | |
| Kidney Margin (OD04339) | 41.2 | Stomach Margin 9060396 | 0.0 |
| Kidney Ca, Clear cell type | 0.0 | Gastric Cancer 9060395 | 0.0 |
| (OD04340) | | | |
| Kidney Margin (OD04340) | 39.5 | Stomach Margin 9060394 | 0.0 |
| Kidney Ca, Nuclear grade 3 | 0.0 | Gastric Cancer 064005 | 0.0 |
| (OD04348) | | | |

TABLE FG
Panel 4.1D

| | Rel. Exp. (%) | | Rel. Exp. (%) |
|---|---|---|---|
| | Ag3243, | | Ag3243, |
| Tissue Name | Run 202013056 | Tissue Name | Run 202013056 |
| Secondary Th1 act | 0.0 | HUVEC IL-1beta | 0.0 |
| Secondary Th2 act | 0.0 | HUVEC IFN gamma | 1.6 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 0.0 |
| | | IL-1beta | |
| Primary Th2 act | 0.0 | Microvascular Dermal EC none | 0.0 |
| Primary Tr1 act | 0.0 | Microsvasular Dermal EC TNFalpha + | 0.0 |
| | | IL-1beta | |
| Primary Th1 rest | 0.0 | Bronchial epithelium TNFalpha + | 0.0 |
| | | IL-1beta | |
| Primary Th2 rest | 0.0 | Small airway epithelium none | 0.0 |
| Primary Tr1 rest | 0.0 | Small airway epithelium TNFalpha + | 0.0 |
| | | IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNFalpha + | 0.0 |
| act | | IL-1beta | |
| CD8 lymphocyte act | 0.0 | Astrocytes rest | 0.0 |
| Secondary CD8 lymphocyte | 0.0 | Astrocytes TNFalpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) PMA/ionomycin | 0.0 |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN gamma | 0.0 | NCI-H292 IL-4 | 0.0 |
| LAK cells IL-2 + IL-18 | 0.0 | NCI-H292 IL-9 | 0.0 |
| LAK cells PMA/ionomycin | 0.0 | NCI-H292 IL-13 | 0.0 |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1beta | 0.0 |
| Two Way MLR 7 day | 0.0 | Lung fibroblast none | 0.0 |
| PBMC rest | 0.0 | Lung fibroblast TNFalpha + IL-1 | 0.0 |
| | | beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 rest | 0.0 |
| B lymphocytes CD40L and | 0.0 | Dermal fibroblast CCD1070 TNF | 0.0 |
| IL-4 | | alpha | |
| EOL-1 dbcAMP | 0.0 | Dermal fibroblast CCD1070 IL-1 | 0.0 |
| | | beta | |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 0.0 |
| Monocytcs LPS | 0.0 | Colon | 0.0 |
| Macrophages rest | 0.0 | Lung | 7.2 |
| Macrophages LPS | 0.0 | Thymus | 0.0 |
| HUVEC none | 0.0 | Kidney | 100.0 |
| HUVEC starved | 0.0 | | |

TABLE FH
Panel 4D

| | Rel. Exp. (%) | | Rel. Exp. (%) |
|---|---|---|---|
| | Ag3243, | | Ag3243, |
| Tissue Name | Run 164390552 | Tissue Name | Run 164390552 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL-4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 0.0 |
| | | IL-1beta | |
| Primary Th2 act | 0.0 | Microvascular Dermal EC none | 0.0 |
| Primary Tr1 act | 0.0 | Microsvasular Dermal EC | 0.0 |
| | | TNFalpha + IL-1beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium TNFalpha + | 0.0 |
| | | IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNFalpha + | 0.0 |
| act | | IL-1beta | |
| CD8 lymphocyte act | 0.0 | Astrocytes rest | 0.0 |
| Secondary CD8 lymphocyte | 0.0 | Astrocytes TNFalpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-M292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNFalpha + IL-1beta | 0.0 |
| PBMC rest | 0.0 | Lung fibroblast none | 0.0 |
| PBMC PWM | 0.0 | Lung fibroblast TNFalpha + IL-1 | 0.0 |
| | | beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 rest | 0.0 |
| IL-4 | | | |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL-1 | 0.0 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 3.2 |
| Macrophages rest | 0.0 | Lung | 18.2 |
| Macrophages LPS | 0.0 | Thymus | 100.0 |
| HUVEC none | 0.0 | Kidney | 3.8 |
| HUVEC starved | 0.0 | | |

CNS_neurodegeneration_v1.0 Summary: Ag3243 This panel confirms the expression of the CG57452-01 gene at low levels in the brain in an independent group of individuals. Interestingly, expression of this gene appears to be slightly down-regulated in the temporal cortex of Alzheimer's disease patients. Therefore, up-regulation of this gene or its protein product may be of use in reversing the dementia/memory loss and neuronal death associated with Alzheimer's disease.

General_screening_panel_v1.4 Summary: Ag3243 Expression of the CG57452-01 gene is highest in fetal lung (CT=30) and fetal kidney (CT=30.4). Therefore, expression of this gene may be used to distinguish these samples from the other samples on this panel.

This gene is also expressed at moderate levels throughout the central nervous system (including in the amygdala, hippocampus, substantia nigra, thalamus, cerebellum, cerebral cortex, and spinal cord) and in one brain cancer line. This gene encodes a protein that is almost identical to protocadherin 15, a cell-adhesion molecule and member of the cadherin family of proteins. Cadherins can act as axon guidance and cell adhesion proteins, specifically during development and in the response to injury (ref. 1-2). Therefore, manipulation of levels of this protein may be of use in inducing a compensatory synaptogenic response to neuronial death in Alzheimer's disease, Parkinson's disease, Huntington's disease, spilocerebellar ataxia, progressive supranuclear palsy, ALS, head trauma, stroke, or any other disease/condition associated with neuronal loss.

Among tissues with metabolic or endocrine function, this gene is moderately expressed in pituitary gland and adrenal gland. This expression suggests that this gene product may play a role in normal metabolic and neuroendocrine function and that dysregulated expression of this gene may contribute to metabolic diseases (such as obesity and diabetes) or neuroendocrine disorders.

References:

1. Ranscht B. (2000) Cadherins: molecular codes for axon guidance and synapse formation. Int. J. Dev. Neurosci. 18: 643-651.

The formation of the myriad of neuronal connections within the vertebrate nervous system relies on expression of molecular tags that match extending axon populations with synaptic target sites. Recent work suggests that cadherins, a group of calcium-dependent cell adhesion molecules, are candidates to serve such a role. The diversity of the cadherin family in the nervous system allows for a multitude of interactions to specify neuronal connections. Specific cadherin types demarcate subpopulations of developing axons that interconnect within neuronal circuits. Expression of different cadherin species at select synapse populations raises exciting prospects for this molecule class in controlling adhesive interactions during synaptogenesis and plasticity. Regulation of cadherin-mediated adhesive strength is an attractive mechanism to explain the different cadherin functions in axon growth and at synapses.

PMID: 10978842

2. Hilschmann N, Barnikol H U, Barnikol-Watanabe S, Gotz H, Kratzin H, Thinnes F P. The immunoglobulin-like genetic predetermination of the brain: the protocadherins, blueprint of the neuronal network. Naturwissenschaften 2001 January;88(1):2-12

The morphogenesis of the brain is governed by synaptogenesis. Synaptogenesis in turn is determined by cell adhesion molecules, which bridge the synaptic cleft and, by homophilic contact, decide which neurons are connected and which are not. Because of their enormous diversification in specificities, protocadherins (pcdh alpha, pcdh beta, pcdh gamma), a new class of cadherins, play a decisive role. Surprisingly, the genetic control of the protocadherins is very similar to that of the immunoglobulins. There are three sets of variable (V) genes followed by a corresponding constant (C) gene. Applying the rules of the immunoglobulin genes to the protocadherin genes leads, despite of this similarity, to quite different results in the central nervous system. The lymphocyte expresses one single receptor molecule specifically directed against an outside stimulus. In contrast, there are three specific recognition sites in each neuron, each expressing a different protocadherin. In this way, 4,950 different neurons arising from one stem cell form a neuronal network, in which homophilic contacts can be formed in 52 layers, permitting an enormous number of different connections and restraints between neurons. This network is one module of the central computer of the brain. Since the V-genes are generated during evolution and V-gene translocation during embryogenesis, outside stimuli have no influence on this network. The network is an inborn property of the protocadherin genes. Every circuit produced, as well as learning and memory, has to be based on this genetically predetermined network. This network is so universal that it can cope with everything, even the unexpected. In this respect the neuronal network resembles the recognition sites of the immunoglobulins.

PMID: 11261353

Panel 1.2 Summary: Ag878 The CG57452-01 gene encodes a protein that is almost identical to protocadherin 15, a cell-adhesion molecule and member of the cadherin family of proteins. Expression of this gene is highest in pituitary gland (CT=28.0) and adrenal gland (CT=28.6). Moderate expression of this gene is also seen in kidney, brain, and lung, consistent with what has been recently reported for the protocadherin 15 gene (ref 1). Expression of this gene is seen throughout the central nervous system, including in amygdala, hippocampus, substantia nigra, thalamus, cerebellum, cerebral cortex, and spinal cord. Mutations of the protocadherin gene PCDH15 have recently been shown to cause Usher syndrome type 1F (ref 2). Therefore, therapeutic modulation of the CG57452-01 protocadherin protein may be of benefit in the treatment of Usher syndrome type 1F, deafness and retinitis pigmentosa. These results are consistent with what is observed in General_screening_panel_v1.4; please see this panel for additional information regarding the potential role of the protocadherin 15 gene in the CNS and metabolic/endocrine function.

References:

I. Murcia C L, Woychik P P. Expression of Pcdh15 in the inner ear, nervous system and various epithelia of the developing embryo. Mech Dev 2001 July; 105(1-2): 163-6.

Protocadherin 15 (Pcdh5) is associated with the Ames waltzer mutation in the mouse. Here we describe where the Pcdh15 gene is expressed at specific times during mouse development using RNA in situ hybridization. The expression of Pcdh15 is found in the sensory epithelium in the developing inner ear, in Rathke's pouch, and broadly throughout the brain with the highest level of expression being detected at embryonic day 16 (E16). Pcdh15 transcripts are also found in the developing eye, dorsal root ganglion, and the dorsal aspect of the neural tube, floor plate and ependymal cells adjacent to the neural canal. Additionally, expression is also detected in the developing glomeruli of the kidney, surface of the tongue, vibrissae, bronchi of the lung, and in the epithelium of the olfactory apparatus, gut and lung.

2. Ahmed Z M, Riazuddin S, Bernstein S L, Ahmed Z, Khan S, Griffith A J, Morell R J, Friedman T B, Riazuddin S, Wilcox E R. Mutations of the protocadherin gene PCDH15 cause Usher syndrome type 1F. Am J Hum Genet 2001 July;69(1):25-34

Human chromosome 10q21-22 harbors USH1F in a region of conserved synteny to mouse chromosome 10. This region of mouse chromosome 10 contains Pcdh15, encoding a protocadherin gene that is mutated in ames waltzer and causes deafness and vestibular dysfunction. Here we report two mutations of protocadherin 15 (PCDH15) found in two families segregating Usher syndrome type 1F. A Northern blot probed with the PCDH15 cytoplasmic domain showed expression in the retina, consistent with its pathogenetic role in the retinitis pigmentosa associated with USH1F.

PMID: 11398101

Panel 2.2 Summary: Ag3243 Expression of this gene is seen at low levels in normal lung and kidney. Interestingly, expression is much lower in lung and kidney tumor samples than in the matched adjacent normal tissue. Therefore, expression of this gene could be used to distinguish normal lung and kidney from lung and kidney tumors. Furthermore, therapeutic modulation of the activity or amount of this gene product using protein therapeutics may be of benefit in the treatment of lung and kidney cancer.

Panel 4.1D Summary: Ag3243 The CG57452-01 gene is only expressed at detectable levels in the kidney (CT=32.4). Therefore, antibody or small molecule therapies designed with the protein encoded for by this gene could modulate kidney function and be important in the treatment of inflammatory or autoimmune diseases that affect the kidney, including lupus and glomerulonephritis.

Panel 4D Summary: Ag3243 The CG57452-01 transcript is expressed at significant levels only in the thymus (CT=33.1) in both runs. The putative protocadherin encoded for by the CG57452-01 gene could therefore play an important role in T cell development. Small molecule therapeutics, or antibody therapeutics designed against the protocadherin encoded for by this gene could be utilized to modulate immune function (T cell development) and be important for organ transplant, AIDS treatment or post chemotherapy immune reconstitiution.

NOV9

Expression of NOV9/CG57625-01 was assessed using the primer-probe set Ag3297, described in Table GA. Results of the RTQ-PCR runs are shown in Tables GB, GC, GD and GE.

TABLE GA
Probe Name Ag3297

| Primers | Sequences | Length | Start Position |
|---|---|---|---|
| Forward | 5′-tgtgaatgtggaggatgctaa-3′ | 21 | 4696 |
| Probe | TET-5′-tgatcacagtccttattttaccaaccca-3′-TAMRA | 28 | 4717 |
| Reverse | 5′-gattcaaacacagacgcttca-3′ | 21 | 4750 |

TABLE GB
CNS_neurodegeneration_v1.0

| | Rel. Exp. (%) | | Rel. Exp. (%) |
|---|---|---|---|
| | Ag3297, | | Ag3297, |
| Tissue Name | Run 210063001 | Tissue Name | Run 210063001 |
| AD 1 Hippo | 4.5 | Control (Path) 3 Temporal Ctx | 1.7 |
| AD 2 Hippo | 10.2 | Control (Path) 4 Temporal Ctx | 18.4 |
| AD 3 Hippo | 2.3 | AD 1 Occipital Ctx | 9.3 |
| AD 4 Hippo | 2.1 | AD 2 Occipital Ctx | 0.0 |
| | | (Missing) | |
| AD 5 hippo | 100.0 | AD 3 Occipital Ctx | 2.5 |
| AD 6 Hippo | 22.1 | AD 4 Occipital Ctx | 12.1 |
| Control 2 Hippo | 19.1 | AD 5 Occipital Ctx | 32.1 |
| Control 4 Hippo | 3.2 | AD 6 Occipital Ctx | 44.8 |
| Control (Path) 3 Hippo | 2.6 | Control 1 Occipital Ctx | 0.5 |
| AD 1 Temporal Ctx | 4.5 | Control 2 Occipital Ctx | 43.5 |
| AD 2 Temporal Ctx | 16.7 | Control 3 Occipital Ctx | 11.5 |
| AD 3 Temporal Ctx | 1.5 | Control 4 Occipital Ctx | 2.4 |
| AD 4 Temporal Ctx | 12.6 | Control (Path) 1 Occipital Ctx | 49.7 |
| AD 5 Inf Temporal Ctx | 72.7 | Control (Path) 2 Occipital Ctx | 10.0 |
| AD 5 Sup Temporal Ctx | 35.8 | Control (Path) 3 Occipital Ctx | 0.6 |
| AD 6 Inf Temporal Ctx | 21.3 | Control (Path) 4 Occipital Ctx | 11.3 |
| AD 6 Sup Temporal Ctx | 27.7 | Control 1 Parietal Ctx | 4.3 |
| Control 1 Temporal Ctx | 3.1 | Control 2 Parietal Ctx | 25.0 |
| Control 2 Temporal Ctx | 22.1 | Control 3 Parietal Ctx | 11.7 |
| Control 3 Temporal Ctx | 9.9 | Control (Path) 1 Parietal | 51.1 |
| | | Ctx | |
| Control 4 Temporal Ctx | 5.9 | Control (Path) 2 Parietal Ctx | 22.4 |
| Control (Path) 1 Temporal | 46.3 | Control (Path) 3 Parietal Ctx | 1.8 |
| Ctx | | | |
| Control (Path) 2 Temporal | 27.7 | Control (Path) 4 Parietal Ctx | 24.7 |
| Ctx | | | |

TABLE GC
General_screening_panel_v1.4

| | Rel. Exp. (%) | | Rel. Exp. (%) |
|---|---|---|---|
| | Ag3297, | | Ag3297, |
| Tissue Name | Run 215669728 | Tissue Name | Run 215669728 |
| Adipose | 0.9 | Renal ca. TK-10 | 0.0 |
| Melanoma* Hs688(A).T | 1.5 | Bladder | 1.1 |
| Melanoma* Hs688(B).T | 3.8 | Gastric ca. (liver met.) NCI- | 6.9 |
| | | N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell carcinoma | 0.0 | Colon ca.* (SW480 met) SW620 | 0.0 |
| SCC-4 | | | |
| Testis Pool | 2.9 | Colon ca. HT29 | 0.0 |
| Prostate ca.* (bone met) | 0.0 | Colon ca. HCT-116 | 0.0 |
| PC-3 | | | |
| Prostate Pool | 6.8 | Colon ca. CaCo-2 | 0.0 |
| Placenta | 0.1 | Colon cancer tissue | 0.7 |
| Uterus Pool | 0.9 | Colon ca. SW1116 | 0.0 |
| Ovarian ca. OVCAR-3 | 4.8 | Colon ca. Colo-205 | 0.0 |
| Ovarian ca. SK-OV-3 | 1.0 | Colon ca. SW-48 | 0.0 |
| Ovarian ca OVCAR-4 | 8.7 | Colon Pool | 1.7 |
| Ovarian ca. OVCAR-5 | 0.2 | Small Intestine Pool | 1.8 |
| Ovarian ca. IGROV-1 | 1.8 | Stomach Pool | 0.9 |
| Ovarian ca. OVCAR-8 | 0.9 | Bone Marrow Pool | 0.5 |
| Ovary | 0.6 | Fetal Heart | 1.6 |
| Breast ca. MCF-7 | 0.0 | Heart Pool | 0.7 |
| Breast ca. MDA-MB-231 | 0.0 | Lymph Node Pool | 5.2 |
| Breast ca. BT 549 | 0.0 | Fetal Skeletal Muscle | 0.5 |
| Breast ca. T47D | 0.2 | Skeletal Muscle Pool | 0.2 |
| Breast ca. MDA-N | 1.2 | Spleen Pool | 0.2 |
| Breast Pool | 1.2 | Thymus Pool | 1.7 |
| Trachea | 0.6 | CNS cancer (glio/astro) U87-MG | 0.0 |
| Lung | 1.4 | CNS cancer (glio/astro) U-118-MG | 0.2 |
| Fetal Lung | 99.3 | CNS cancer (neuro; met) SK-N-AS | 0.0 |
| Lung ca. NCI-N417 | 0.0 | CNS cancer (astro) SF-539 | 2.0 |
| Lung ca. LX-1 | 0.0 | CNS cancer (astro) SNB-75 | 6.9 |
| Lung ca. NCI-H146 | 0.1 | CNS cancer (glio) SNB-9 | 1.9 |
| Lung ca. SHP-77 | 7.0 | CNS cancer (glio) SF-295 | 0.0 |
| Lung ca. A549 | 2.0 | Brain (Amygdala) Pool | 14.3 |
| Lung ca. NCI-H526 | 40.6 | Brain (cerebellum) | 3.4 |
| Lung ca. NCI-H23 | 0.6 | Brain (fetal) | 100.0 |
| Lung ca. NCI-H460 | 0.0 | Brain (Hippocampus) Pool | 14.7 |
| Lung ca. HOP-62 | 0.0 | Cerebral Cortex Pool | 23.8 |
| Lung ca. NCI-H522 | 0.0 | Brain (Substantia nigra) Pool | 15.2 |
| Liver | 0.1 | Brain (Thalamus) Pool | 21.3 |
| Fetal Liver | 1.4 | Brain (whole) | 23.3 |
| Liver ca. HepG2 | 0.0 | Spinal Cord Pool | 7.5 |
| Kidney Pool | 3.4 | Adrenal Gland | 0.2 |
| Fetal Kidney | 12.3 | Pituitary gland Pool | 1.7 |
| Renal ca. 786-0 | 0.1 | Salivary Gland | 0.0 |
| Renal ca. A498 | 0.9 | Thyroid (female) | 0.1 |
| Renal ca. ACHN | 0.0 | Pancreatic ca. CAPAN2 | 0.0 |
| Renal ca. UO-31 | 0.5 | Pancreas Pool | 0.5 |

TABLE GD
Panel 4D

| | Rel. Exp. (%) | | Rel. Exp. (%) |
|---|---|---|---|
| | Ag3297, | | Ag3297, |
| Tissue Name | Run 164633943 | Tissue Name | Run 164633943 |
| Secondary Th1 act | 0.1 | HUVEC IL-1beta | 0.1 |
| Secondary Th2 act | 0.2 | HUVEC IFN gamma | 1.3 |
| Secondary Tr1 act | 0.4 | HUVEC TNFalpha + IFN gamma | 0.5 |
| Secondary Th1 rest | 0.4 | HUVEC TNFalpha + IL4 | 0.4 |
| Secondary Th2 rest | 0.0 | HUVEC IL-11 | 0.2 |
| Secondary Tr1 rest | 0.1 | Lung Microvascular EC none | 0.6 |
| Primary Th1 act | 0.1 | Lung Microvascular EC TNFalpha + | 0.1 |
| | | IL-1beta | |
| Primary Th2 act | 0.5 | Microvascular Dermal EC none | 1.2 |
| Primary Tr1 act | 0.4 | Microsvasular Dermal EC | 0.2 |
| | | TNFalpha + IL-1beta | |
| Primary Th1 rest | 0.3 | Bronchial epithelium TNFalpha + | 0.8 |
| | | IL1beta | |
| Primary Th2 rest | 0.1 | Small airway epithelium none | 0.7 |
| Primary Tr1 rest | 0.0 | Small airway epithelium TNFalpha + | 1.9 |
| | | IL-1beta | |
| CD45RA CD4 lymphocyte | 0.2 | Coronery artery SMC rest | 2.2 |
| act | | | |
| CD45RO CD4 lymphocyte | 0.1 | Coronery artery SMC TNFalpha + | 2.3 |
| act | | IL-1beta | |
| CD8 lymphocyte act | 0.1 | Astrocytes rest | 100.0 |
| Secondary CD8 lymphocyte | 1.1 | Astrocytes TNFalpha + IL-1beta | 28.9 |
| rest | | | |
| Secondary CD8 lymphocyte | 1.6 | KU-812 (Basophil) rest | 2.9 |
| act | | | |
| CD4 lymphocyte none | 0.4 | KU-812 (Basophil) PMA/ionomycin | 15.2 |
| 2ry Th1/Th2/Tr1_anti-CD95 | 1.5 | CCD1106 (Keratinocytes) none | 0.1 |
| CH11 | | | |
| LAK cells rest | 0.4 | CCD1106 (Keratinocytes) | 0.5 |
| | | TNFalpha + IL-1beta | |
| LAK cells IL-2 | 1.7 | Liver cirrhosis | 2.6 |
| LAK cells IL-2 + IL-12 | 1.4 | Lupus kidney | 2.0 |
| LAK cells IL-2 + IFN gamma | 3.5 | NCI-H292 none | 1.0 |
| LAK cells IL-2 + IL-18 | 1.1 | NCI-H292 IL-4 | 4.7 |
| LAK cells PMA/ionomycin | 0.1 | NCI-H292 IL-9 | 1.1 |
| NK Cells IL-2 rest | 0.8 | NCI-H292 IL-13 | 1.7 |
| Two Way MLR 3 day | 0.3 | NCI-H292 IFN gamma | 0.6 |
| Two Way MLR 5 day | 1.0 | HPAEC none | 0.6 |
| Two Way MLR 7 day | 0.4 | HPAEC TNF alpha + IL-1beta | 0.2 |
| PBMC rest | 0.3 | Lung fibroblast none | 8.7 |
| PBMC PWM | 1.0 | Lung fibroblast TNF alpha + | 2.3 |
| | | IL-1beta | |
| PBMC PHA-L | 1.0 | Lung fibroblast IL-4 | 18.7 |
| Ramos (B cell) none | 0.7 | Lung fibroblast IL-9 | 18.8 |
| Ramos (B cell) ionomycin | 2.2 | Lung fibroblast IL-13 | 14.7 |
| B lymphocytes PWM | 1.2 | Lung fibroblast IFN gamma | 12.2 |
| B lymphocytes CD40L andIL-4 | 2.1 | Dermal fibroblast CCD1070 rest | 18.4 |
| EOL-1 dbcAMP | 0.5 | Dermal fibroblast CCD1070 TNF | 16.2 |
| | | alpha | |
| EOL-1 dbcAMP | 0.4 | Dermal fibroblast CCD1070 IL-1 | 3.1 |
| PMA/ionomycin | | beta | |
| Dendritic cells none | 0.4 | Dermal fibroblast IFN gamma | 0.3 |
| Dendritic cells LPS | 0.3 | Dermal fibroblast IL-4 | 0.0 |
| Dendritic cells anti-CD40 | 1.3 | IBD Colitis 2 | 1.2 |
| Monocytes rest | 0.4 | IBD Crohn's | 0.5 |
| Monocytes LPS | 0.3 | Colon | 0.3 |
| Macrophages rest | 0.4 | Lung | 5.5 |
| Macrophages LPS | 0.7 | Thymus | 2.2 |
| HUVEC none | 0.8 | Kidney | 4.4 |
| HUVEC starved | 6.9 | | |

TABLE GE
Panel CNS_1.1

| | Rel. Exp. (%) | | Rel. Exp. (%) |
|---|---|---|---|
| | Ag3297, | | Ag3297, |
| Tissue Name | Run 204173323 | Tissue Name | Run 204173323 |
| Cing Gyr Depression2 | 5.1 | BA17 PSP2 | 14.9 |
| Cing Gyr Depression | 10.1 | BA17 PSP | 46.3 |
| Cing Gyr PSP2 | 1.5 | BA17 Huntington's2 | 10.1 |
| Cing Gyr PSP | 11.8 | BA17 Huntington's | 32.3 |
| Cing Gyr Huntington's2 | 13.4 | BA17 Parkinson's2 | 54.7 |
| Cing Gyr Huntington's | 48.3 | BA17 Parkinson's | 37.6 |
| Cing Gyr Parkinson's2 | 37.4 | BA17 Alzheimer's2 | 7.0 |
| Cing Gyr Parkinson's | 47.3 | BA17 Control2 | 48.3 |
| Cing Gyr Alzheimer's2 | 4.5 | BA17 Control | 69.3 |
| Cing Gyr Alzheimer's | 16.3 | BA9 Depression2 | 6.6 |
| Cing Gyr Control 2 | 30.8 | BA9 Depression | 9.2 |
| Cing Gyr Control | 66.0 | BA9 PSP2 | 6.5 |
| Temp Pole Depression2 | 5.6 | BA9 PSP | 18.4 |
| Temp Pole PSP2 | 3.0 | BA9 Huntington's2 | 8.7 |
| Temp Pole PSP | 4.4 | BA9 Huntington's | 61.6 |
| Temp Pole Huntington's | 22.1 | BA9 Parkinson's2 | 64.6 |
| Temp Pole Parkinson's2 | 24.8 | BA9 Parkinson's | 46.7 |
| Temp Pole Parkinson's | 32.8 | BA9 Alzheimer's2 | 11.3 |
| Temp Pole Alzheimer's2 | 3.3 | BA9 Alzheimer's | 7.4 |
| Temp Pole Alzheimer's | 5.1 | BA9 Control2 | 69.3 |
| Temp Pole Control2 | 51.8 | BA9 Control | 27.4 |
| Temp Pole Control | 27.5 | BA7 Depression | 10.9 |
| Glob Palladus Depression | 3.6 | BA7 PSP2 | 27.9 |
| Glob Palladus PSP2 | 5.5 | BA7 PSP | 56.3 |
| Glob Palladus PSP | 6.7 | BA7 Huntington's2 | 21.3 |
| Glob Palladus | 11.7 | BA7 Huntington's | 49.7 |
| Parkinson's2 | | | |
| Glob Palladus Parkinson's | 97.9 | BA7 Parkinson's2 | 50.7 |
| Glob Palladus | 10.9 | BA7 Parkinson's | 20.3 |
| Alzheimer's2 | | | |
| Glob Palladus Alzheimer's | 9.8 | BA7 Alzheimer's2 | 4.3 |
| Glob Palladus Control2 | 24.5 | BA7 Control2 | 25.3 |
| Glob Palladus Control | 23.2 | BA7 Control | 45.4 |
| Sub Nigra Depression2 | 5.9 | BA4 Depression2 | 6.4 |
| Sub Nigra Depression | 3.3 | BA4 Depression | 14.5 |
| Sub Nigra PSP2 | 5.1 | BA4 PSP2 | 36.3 |
| Sub Nigra Huntington's2 | 5.8 | BA4 PSP | 12.0 |
| Sub Nigra Huntington's | 74.2 | BA4 Huntington's2 | 5.4 |
| Sub Nigra Parkinson's2 | 39.5 | BA4 Huntington's | 42.0 |
| Sub Nigra Alzheimer's2 | 3.8 | BA4 Parkinson's2 | 100.0 |
| Sub Nigra Control2 | 12.7 | BA4 Parkinson's | 52.9 |
| Sub Nigra Control | 45.1 | BA4 Alzheimer's2 | 3.0 |
| BA17 Depression2 | 11.0 | BA4 Control2 | 53.2 |
| BA17 Depression | 7.1 | BA4 Control | 36.3 |

CNS_neurodegeneration_v1.0 Summary: Ag3297 This panel confirms the expression of this gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3297 The CG57625-01 gene is expressed at low to moderate levels in many of the tissues on this panel, with the highest levels of expression in fetal brain and fetal lung (CT=27.4). Interestingly, the levels of expression are significantly lower in the sample from adult lung (CT=33.5) than in fetal lung. Therefore, expression of this gene can be used to distinguish fetal lung from adult lung.

This gene is expressed at low levels throughout the CNS, including in amygdala, substantia nigra, thalamus, cerebellum, cerebral cortex, and spinal cord. The CG57625-01 gene encodes a protein with homology to protocadherin, a cell-adhesion molecule and member of the cadherin family of proteins. Cadherins can act as axon guidance and cell adhesion proteins, specifically during development and in the response to injury (ref. 1-2). Therefore, manipulation of levels of this protein may be of use in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss.

This gene is also expressed at low to moderate levels in a number of tissues with metabolic or endocrine function, including heart, gastrointestinal tract, fetal liver, pituitary gland, and adipose. Therefore, therapeutic modulation of the activity of this gene may prove useful in the treatment of endocrine/metabolically related diseases, such as obesity and diabetes.

Additionally, this gene is also expressed at significant levels in lung cancer, ovarian cancer, gastric cancer and melanoma cell lines. Hence expression of this gene could be used as a diagnostic marker for these types of cancers. Furthermore, therapeutic modulation of the activity of this gene or its protein product, using small molecule drugs, antibodies, or protein therapeutics, may be of use in the treatment of lung cancer, ovarian cancer, gastric cancer and melanoma.

References:

1. Ranscht B. (2000) Cadherins: molecular codes for axon guidance and synapse formation. Int. J. Dev. Neurosci. 18: 643-651.

The formation of the myriad of neuronal connections within the vertebrate nervous system relies on expression of molecular tags that match extending axon populations with synaptic target sites. Recent work suggests that cadherins, a group of calcium-dependent cell adhesion molecules, are candidates to serve such a role. The diversity of the cadherin family in the nervous system allows for a multitude of interactions to specify neuronal connections. Specific cadherin types demarcate subpopulations of developing axons that interconnect within neuronal circuits. Expression of different cadherin species at select synapse populations raises exciting prospects for this molecule class in controlling adhesive interactions during synaptogenesis and plasticity. Regulation of cadherin-mediated adhesive strength is an attractive mechanism to explain the different cadherin functions in axon growth and at synapses.

PMID: 10978842

2. Hilschmann N, Barnikol H U, Barnikol-Watanabe S, Gotz H, Kratzin H, Thinnes F P. The immunoglobulin-like genetic predetermination of the brain: the protocadherins, blueprint of the neuronal network. Naturwissenschaften 2001 January;88(1):2-12

The morphogenesis of the brain is governed by synaptogenesis. Synaptogenesis in turn is determined by cell adhesion molecules, which bridge the synaptic cleft and, by homophilic contact, decide which neurons are connected and which are not. Because of their enormous diversification in specificities, protocadherins (pcdh alpha, pcdh beta, pcdh gamma), a new class of cadherins, play a decisive role. Surprisingly, the genetic control of the protocadherins is very similar to that of the immunoglobulins. There are three sets of variable (V) genes followed by a corresponding constant (C) gene. Applying the rules of the immunoglobulin genes to the protocadherin genes leads, despite of this similarity, to quite different results in the central nervous system. The lymphocyte expresses one single receptor molecule specifically directed against an outside stimulus. In contrast, there are three specific recognition sites in each neuron, each expressing a different protocadherin. In this way, 4,950 different neurons arising from one stem cell form a neuronal network, in which homophilic contacts can be formed in 52 layers, permitting an enormous number of different connections and restraints between neurons. This network is one module of the central computer of the brain. Since the V-genes are generated during evolution and V-gene translocation during embryogenesis, outside stimuli have no influence on this network. The network is an inborn property of the protocadherin genes. Every circuit produced, as well as learning and memory, has to be based on this genetically predetermined network. This network is so universal that it can cope with everything, even the unexpected. In this respect the neuronal network resembles the recognition sites of the immunoglobulins.

PMID: 11261353

Panel 4D Summary: Ag3297 Expression of the CG57625-01 gene is highest in resting astrocytes (CT=28), consistent with the expression of this gene in the brain in General_screening_panel_v1.4. This gene is also moderately expressed in lung and dermal fibroblasts, irrespective of treatment. Therefore, therapeutic modulation of the activity of this gene or its protein product may be of benefit in the treatment of asthma, emphysema, and psoriasis. Low levels of expression of this gene are detected in a number of other samples on this panel.

Panel CNS_{—}1.1 Summary: Ag3297 This panel confirms the expression of this gene at low to moderate levels in the brain in an independent group of individuals. Please see Panel 1.4 for a discussion of the potential utility of this gene in treatment of central nervous system disorders.

NOV10

Expression of NOV10/CG57553-01 was assessed using the primer-probe set Ag3283, described in Table HA. Results of the RTQ-PCR runs are shown in Tables HB, HC and HD.

TABLE HA
Probe Name Ag3283

| Primers | Sequences | Length | Start Position | SEQ ID NO |
|---|---|---|---|---|
| Forward | 5′-gcctccaatatgaaacttcaaa-3′ | 221 | 461 | 276 |
| Probe | TET-5′-tcctcagctacgagtaagttctgtctca-3′-TAMRA | 28 | 504 | 277 |
| Reverse | 5′-cagaaccatcaggttgtgattt-3′ | 22 | 532 | 278 |

TABLE HB
CNS_neurodegeneration_v1.0

| | Rel. Exp. (%) | | Rel. Exp. (%) |
|---|---|---|---|
| | Ag3283, | | Ag3283, |
| Tissue Name | Run 210060840 | Tissue Name | Run 210060840 |
| AD 1 Hippo | 13.3 | Control (Path) 3 Temporal Ctx | 4.5 |
| AD 2 Hippo | 14.1 | Control (Path) 4 Temporal Ctx | 44.1 |
| AD 3 Hippo | 15.4 | AD 1 Occipital Ctx | 19.1 |
| AD 4 Hippo | 7.6 | AD 2 Occipital Ctx (Missing) | 0.0 |
| AD 5 Hippo | 76.8 | AD 3 Occipital Ctx | 15.1 |
| AD 6 Hippo | 65.1 | AD 4 Occipital Ctx | 22.7 |
| Control 2 Hippo | 25.2 | AD 5 Occipital Ctx | 41.8 |
| Control 4 Hippo | 10.7 | AD 6 Occipital Ctx | 26.4 |
| Control (Path) 3 Hippo | 13.8 | Control 1 Occipital Ctx | 6.2 |
| AD 1 Temporal Ctx | 34.9 | Control 2 Occipital Ctx | 35.8 |
| AD 2 Temporal Ctx | 40.3 | Control 3 Occipital Ctx | 23.7 |
| AD 3 Temporal Ctx | 9.2 | Control 4 Occipital Ctx | 11.1 |
| AD 4 Temporal Ctx | 18.4 | Control (Path) 1 Occipital Ctx | 90.1 |
| AD 5 Inf Temporal Ctx | 100.0 | Control (Path) 2 Occipital Ctx | 12.3 |
| AD 5 Sup Temporal Ctx | 51.1 | Control (Path) 3 Occipital Ctx | 5.3 |
| AD 6 Inf Temporal Ctx | 76.8 | Control (Path) 4 Occipital Ctx | 23.0 |
| AD 6 Sup Temporal Ctx | 93.3 | Control 1 Parietal Ctx | 2.9 |
| Control 1 Temporal Ctx | 6.7 | Control 2 Parietal Ctx | 65.1 |
| Control 2 Temporal Ctx | 36.3 | Control 3 Parietal Ctx | 18.3 |
| Control 3 Temporal Ctx | 13.1 | Control (Path) 1 Parietal Ctx | 52.1 |
| Control 3 Temporal Ctx | 8.7 | Control (Path) 2 Parietal Ctx | 28.7 |
| Control (Path) 1 Temporal Ctx | 45.1 | Control (Path) 3 Parietal Ctx | 5.4 |
| Control (Path) 2 Temporal Ctx | 27.7 | Control (Path) 4 Parietal Ctx | 52.1 |

TABLE HC
General_screening_panel_v1.4

| | Rel. Exp. (%) | | Rel. Exp. (%) |
|---|---|---|---|
| | Ag3283, Run | | Ag3283, Run |
| Tissue Name | 216512996 | Tissue Name | 216512996 |
| Adipose | 8.3 | Renal ca. TK-10 | 29.3 |
| Melanoma* Hs688(A).T | 5.0 | Bladder | 25.2 |
| Melanoma* Hs688(B).T | 9.7 | Gastric ca. (liver met.) NCI- | 40.9 |
| | | N87 | |
| Melanoma* M14 | 21.6 | Gastric ca. KATO III | 48.3 |
| Melanoma* LOXIMVI | 12.8 | Colon ca. SW-948 | 9.1 |
| Melanoma* SK-MEL-5 | 14.7 | Colon ca. SW480 | 28.3 |
| Squamous cell carcinoma | 8.4 | Colon ca.* (SW480 met) | 21.2 |
| SCC-4 | | SW620 | |
| Testis Pool | 20.3 | Colon ca. HT29 | 13.4 |
| Prostate ca.* (bone met) | 16.4 | Colon ca. HCT-116 | 37.6 |
| PC-3 | | | |
| Prostate Pool | 6.2 | Colon ca CaCo-2 | 14.2 |
| Placenta | 1.0 | Colon cancer tissue | 13.9 |
| Uterus Pool | 4.1 | Colon ca. SW1116 | 2.6 |
| Ovarian ca. OVCAR-3 | 30.6 | Colon ca Colo-205 | 10.8 |
| Ovarian ca. SK-OV-3 | 61.6 | Colon ca. SW-48 | 6.5 |
| Ovarian ca. OVCAR-4 | 4.9 | Colon Pool | 15.5 |
| Ovarian ca. OVCAR-5 | 28.5 | Small Intestine Pool | 21.5 |
| Ovarian ca. IGROV-1 | 16.0 | Stomach Pool | 13.3 |
| Ovarian ca. OVCAR-8 | 8.8 | Bone Marrow Pool | 5.4 |
| Ovary | 14.8 | Fetal Heart | 10.2 |
| Breast ca. MCF-7 | 24.5 | Heart Pool | 7.9 |
| Breast ca. MDA-MB-23 | 50.3 | Lymph Node Pool | 17.0 |
| Breast ca. BT 549 | 100.0 | Fetal Skeletal Muscle | 6.2 |
| Breast ca. T47D | 47.3 | Skeletal Muscle Pool | 17.7 |
| Breast ca. MDA-N | 14.5 | Spleen Pool | 17.2 |
| Breast Pool | 19.8 | Thymus Pool | 20.6 |
| Trachea | 7.3 | CNS cancer (glio/astro) U87- | 37.4 |
| | | MG | |
| Lung | 7.5 | CNS cancer (glio/astro) U- | 49.7 |
| | | 118-MG | |
| Fetal Lung | 37.6 | CNS cancer (neuro; met) SK-N-AS | 80.7 |
| Lung ca. NCI-N417 | 4.6 | CNS cancer (astro) SF-539 | 11.5 |
| Lung ca. LX-1 | 41.8 | CNS cancer (astro) SNB-75 | 36.3 |
| Lung ca. NCI-H146 | 11.3 | CNS cancer (glio) SNB-19 | 12.4 |
| Lung ca. SHP-77 | 26.6 | CNS cancer (glio) SF-295 | 29.5 |
| Lung ca. A549 | 18.8 | Brain (Amygdala) Pool | 6.3 |
| Lung ca. NCI-H526 | 13.9 | Brain (cerebellum) | 4.4 |
| Lung ca. NCI-H23 | 31.4 | Brain (fetal) | 43.8 |
| Lung ca. NCI-H460 | 17.6 | Brain (Hippocampus) Pool | 14.5 |
| Lung ca. HOP-62 | 16.2 | Cerebral Cortex Pool | 11.4 |
| Lung ca. NCI-H522 | 23.3 | Brain (Substantia nigra) Pool | 7.9 |
| Liver | 0.1 | Brain (Thalamus) Pool | 17.0 |
| Fetal Liver | 22.8 | Brain (whole) | 3.2 |
| Liver ca. HepG2 | 21.5 | Spinal Cord Pool | 11.5 |
| Kidney Pool | 23.3 | Adrenal Gland | 4.0 |
| Fetal Kidney | 40.9 | Pituitary gland Pool | 3.8 |
| Renal ca 786-0 | 57.0 | Salivary Gland | 0.4 |
| Renal ca. A498 | 2.3 | Thyroid (female) | 5.1 |
| Renal ca. ACHN | 11.3 | Pancreatic ca. CAPAN2 | 51.1 |
| Renal ca. UO-31 | 15.0 | Pancreas Pool | 22.2 |

TABLE HD
Panel 4D

| | Rel. Exp. (%) | | Rel. Exp. (%) |
|---|---|---|---|
| | Ag3283, Run | | Ag3283, Run |
| Tissue Name | 164634322 | Tissue Name | 164634322 |
| Secondary Th1 act | 6.9 | HUVEC IL-1beta | 7.2 |
| Secondary Th2 act | 7.3 | HUVEC IFN gamma | 10.7 |
| Secondary Tr1 act | 6.3 | HUVEC TNF alpha + IFN gamma | 2.9 |
| Secondary Th1 rest | 1.8 | HUVEC TNF alpha + IL4 | 5.8 |
| Secondary Th2 rest | 3.4 | HUVEC IL-11 | 3.5 |
| Secondary Tr1 rest | 4.7 | Lung Microvascular EC none | 8.8 |
| Primary Th1 act | 7.6 | Lung Microvascular EC TNFalpha + | 10.7 |
| | | IL-1beta | |
| Primary Th2 act | 5.5 | Microvascular Dermal EC none | 11.6 |
| Primary Tr1 act | 7.4 | Microsvasular Dermal EC | 6.6 |
| | | TNFalpha + IL-1beta | |
| Primary Th1 rest | 23.0 | Bronchial epithelium | 7.8 |
| | | TNFalpha + IL1beta | |
| Primary Th2 rest | 17.9 | Small airway epithelium none | 1.0 |
| Primary Tr1 rest | 8.2 | Small airway epithelium | 18.4 |
| | | TNFalpha + IL-1beta | |
| CD45RA CD4 lymphocyte | 4.9 | Coronery artery SMC rest | 2.5 |
| act | | | |
| CD45RO CD4 lymphocyte | 8.2 | Coronery artery SMC TNFalpha + | 2.8 |
| act | | IL-1beta | |
| CD8 lymphocyte act | 5.8 | Astrocytes rest | 5.9 |
| Secondary CD8 lymphocyte | 3.2 | Astrocytes TNFalpha + IL-1beta | 2.4 |
| rest | | | |
| Secondary CD8 lymphocyte | 6.8 | KU-812 (Basophil) rest | 4.3 |
| act | | | |
| CD4 lymphocyte none | 2.5 | KU-8l2 (Basophil) | 21.8 |
| | | PMA/ionomycin | |
| 2ry Th1/Th2/Tr1_anti-CD95 | 6.5 | CCD1106 (Keratinocytes) none | 6.6 |
| CH11 | | | |
| LAK cells rest | 14.2 | CCD1106 (Keratinocytes) | 2.0 |
| | | TNFalpha + IL-1beta | |
| LAK cells IL-2 | 14.2 | Liver cirrhosis | 1.5 |
| LAK cells IL-2 + IL-12 | 7.4 | Lupus kidney | 1.5 |
| LAK cells IL-2 + IFN gamma | 13.8 | NCI-H292 none | 17.4 |
| LAK cells IL-2 + IL-18 | 9.7 | NCI-H292 IL-4 | 18.9 |
| LAK cells PMA/ionomycin | 5.0 | NCI-H292 IL-9 | 23.5 |
| NK Cells IL-2 rest | 14.4 | NCI-H292 IL-13 | 5.3 |
| Two Way MLR 3 day | 19.2 | NCI-H292 IFN gamma | 8.5 |
| Two Way MLR 5 day | 5.6 | HPAEC none | 6.3 |
| Two Way MLR 7 day | 3.1 | HPAEC TNF alpha + IL-1 beta | 8.0 |
| PBMC rest | 7.4 | Lung fibroblast none | 4.7 |
| PBMC PWM | 24.8 | Lung fibroblast TNF alpha + IL-1 | 1.8 |
| | | beta | |
| PBMC PMA-L | 8.8 | Lung fibroblast IL-4 | 10.4 |
| Ramos (B cell) none | 26.4 | Lung fibroblast IL-9 | 6.7 |
| Ramos (B cell) ionomycin | 100.0 | Lung fibroblast IL-13 | 6.5 |
| B lymphocytes PWM | 22.7 | Lung fibroblast IFN gamma | 7.2 |
| B lymphocytes CD40L and | 21.5 | Dermal fibroblast CCD1070 rest | 25.0 |
| IL-4 | | | |
| EOL-1 dbcAMP | 15.3 | Dermal fibroblast CCD1070 TNF | 56.3 |
| | | alpha | |
| EOL-1 dbcAMP | 27.7 | Dermal fibroblast CCD1070 IL-1 | 7.5 |
| PMA/ionomycin | | beta | |
| Dendritic cells none | 13.5 | Dermal fibroblast IFN gamma | 2.8 |
| Dendritic cells LPS | 6.8 | Dermal fibroblast IL-4 | 8.6 |
| Dendritic cells anti-CD40 | 12.3 | IBD Colitis 2 | 0.4 |
| Monocytes rest | 13.8 | IBD Crohn's | 1.8 |
| Monocytes LPS | 13.4 | Colon | 28.7 |
| Macrophages rest | 12.4 | Lung | 4.4 |
| Macrophages LPS | 10.3 | Thymus | 16.3 |
| HUVEC none | 7.3 | Kidney | 32.5 |
| HUVEC starved | 22.1 | | |

CNS_neurodegeneration_v1.0 Summary: Ag3283 This panel confirms the expression of the CG57553-01 gene at low to moderate levels in the brain in an independent group of individuals. Interestingly, this gene appears to be slightly upregulated in the temporal cortex of Alzheimer's disease patients. Therefore, therapeutic modulation of the activity of this gene or its protein product may be of use to decrease neuronal death in the treatment of Alzheimer's disease or other neurological disorders.

General_screening_panel_v1.4 Summary: Ag3283 Expression of the CG57553-01 gene is highest in a breast cancer cell line (CT=27.2). This gene is expressed at low to moderate levels in a number of tissues with metabolic or endocrine function, including adipose, adrenal gland, gastrointestinal tract, pancreas, skeletal muscle and thyroid. Therefore, therapeutic modulation of the activity of this gene may prove useful in the treatment of endocrine/metabolically related diseases, such as obesity and diabetes. Interestingly, this gene is differentially expressed in adult (CT=36) vs fetal liver (CT=29) and may be useful for distinguishing adult and fetal liver.

This gene is also expressed at moderate levels throughout the CNS, including in amygdala, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

There are also significant levels of expression in clusters of cell lines derived from melanoma, prostate, ovarian, brain, breast, pancreatic, gastric, and liver cancers. This observation suggests that expression of this gene may be associated with these cancers. Therefore, therapeutic modulation of the activity of this gene or its protein product might be of use in the treatment of these cancers.

Panel 4D Summary: Ag3283 The CG57553-01 gene is expressed at low to moderate levels in a number cell types within this panel, with highest expression in activated B cells (CT=27.2). Therefore, therapeutic modulation of the activity of this gene may prove useful in the treatment of rheumatic disease including rheumatoid arthritis, lupus, osteoarthritis, and hyperproliferative B cell disorders.

NOV11

Expression of gene NOV11a/CG57488-01 and variants NOV11B/CG57488-02 and NOV11C/CG57488-03 was assessed using the primer-probe sets Ag3254 and Ag3339, described in Tables IA and IB. Results of the RTQ-PCR runs are shown in Tables IC, ID and IE. Please note that variant CG57488-03 is only recognized by primer-probe set Ag3339.

TABLE IA
Probe Name Ag3254

| Primers | Sequences | Length | Start Position | SEQ ID NO |
|---|---|---|---|---|
| Forward | 5′-cttgtgcctgtggttaacatct-3′ | 22 | 5853 | 279 |
| Probe | TET-5′-tgatcactccaggatattgacacgaa-3′-TAMRA | 26 | 5892 | 280 |
| Reverse | 5′-accccagaacatgtgagtaaga-3′ | 22 | 5931 | 281 |

TABLE IB
Probe Name Ag3339

| Primers | Sequences | Length | Start Position | SEQ ID NO |
|---|---|---|---|---|
| Forward | 5′-ggtcttctacgtcagggagaat-3′ | 122 | 1806 | 282 |
| Probe | TET-5′-cagtttgcagtcgagaccttcttcga-3′-TAMRA | 26 | 1852 | 283 |
| Reverse | 5′-gctgaatacgtcactgaaacct-3′ | 22 | 1883 | 284 |

TABLE IC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3254, Run | Ag3339, Run | | Ag3254, Run | Ag3339, Run |
| Tissue Name | 209990706 | 210139045 | Tissue Name | 209990706 | 210139045 |
| AD 1 Hippo | 0.0 | 19.1 | Control (Path) 3 | 28.9 | 6.2 |
| | | | Temporal Ctx | | |
| AD 2 Hippo | 38.2 | 70.7 | Control (Path) 4 | 0.0 | 3.7 |
| | | | Temporal Ctx | | |
| AD 3 Hippo | 7.3 | 9.1 | AD 1 Occipital | 0.0 | 17.7 |
| | | | Ctx | | |
| AD 4 Hippo | 12.1 | 5.4 | AD 2 Occipital | 0.0 | 0.0 |
| | | | Ctx (Missing) | | |
| AD 5 Hippo | 53.2 | 25.3 | AD 3 Occipital | 18.2 | 19.2 |
| | | | Ctx | | |
| AD 6 Hippo | 5.8 | 100.0 | AD 4 Occipital | 59.9 | 11.7 |
| | | | Ctx | | |
| Control 2 Hippo | 39.2 | 17.7 | AD 5 Occipital | 38.2 | 12.3 |
| | | | Ctx | | |
| Control 4 Hippo | 58.6 | 6.9 | AD 6 Occipital | 0.0 | 22.5 |
| | | | Ctx | | |
| Control (Path) 3 | 16.2 | 10.6 | Control 1 | 0.0 | 7.2 |
| Hippo | | | Occipital Ctx | | |
| AD 1 Temporal | 0.0 | 6.1 | Control 2 | 68.8 | 31.2 |
| Ctx | | | Occipital Ctx | | |
| AD 2 Temporal | 49.0 | 14.7 | Control 3 | 23.3 | 18.0 |
| Ctx | | | Occipital Ctx | | |
| AD 3 Temporal | 8.5 | 3.1 | Control 4 | 27.2 | 31.4 |
| Ctx | | | Occipital Ctx | | |
| AD 4 Temporal | 57.0 | 4.5 | Control (Path) 1 | 33.4 | 34.2 |
| Ctx | | | Occipital Ctx | | |
| AD 5 Inf | 89.5 | 53.6 | Control (Path) 2 | 11.7 | 4.6 |
| Temporal Ctx | | | Occipital Ctx | | |
| AD 5 Sup | 60.7 | 62.4 | Control (Path) 3 | 100.0 | 4.3 |
| Temporal Ctx | | | Occipital Ctx | | |
| AD 6 Inf | 0.0 | 31.6 | Control (Path) 4 | 0.0 | 10.4 |
| Temporal Ctx | | | Occipital Ctx | | |
| AD 6 Sup | 0.0 | 22.4 | Control 1 | 13.0 | 4.7 |
| Temporal Ctx | | | Parietal Ctx | | |
| Control 1 | 0.0 | 2.6 | Control 2 | 61.6 | 20.6 |
| Temporal Ctx | | | Parietal Ctx | | |
| Control 2 | 56.3 | 3.8 | Control 3 | 0.0 | 12.3 |
| Temporal Ctx | | | Parietal Ctx | | |
| Control 3 | 27.2 | 3.3 | Control (Path) 1 | 9.1 | 11.3 |
| Temporal Ctx | | | Parietal Ctx | | |
| Control 3 | 0.0 | 3.9 | Control (Path) 2 | 0.0 | 15.9 |
| Temporal Ctx | | | Parietal Ctx | | |
| Control (Path) 1 | 63.7 | 3.2 | Control (Path) 3 | 68.3 | 7.0 |
| Temporal Ctx | | | Parietal Ctx | | |
| Control (Path) 2 | 16.0 | 1.5 | Control (Path) 4 | 5.8 | 31.6 |
| Temporal Ctx | | | Parietal Ctx | | |

TABLE ID
General_screening_panel_v1.4

| | Rel. Exp. (%) | Rel. Exp. (%) | | Rel. Exp. (%) | Rel. Exp. (%) |
|---|---|---|---|---|---|
| | Ag3254, Run | Ag3339, Run | | Ag3254, Run | Ag3339, Run |
| Tissue Name | 214694852 | 215773747 | Tissue Name | 214694852 | 215773747 |
| Adipose | 0.1 | 0.1 | Renal ca. TK-10 | 0.7 | 0.0 |
| Melanoma* | 0.2 | 0.0 | Bladder | 0.3 | 0.1 |
| Hs688(A).T | | | | | |
| Melanoma* | 0.3 | 0.0 | Gastric ca. (liver | 4.9 | 0.0 |
| Hs688(B).T | | | met.) NCI-N87 | | |
| Melanoma* M14 | 0.0 | 0.0 | Gastric ca. KATO III | 0.0 | 0.0 |
| Melanoma* | 0.2 | 0.0 | Colon ca. SW-948 | 0.0 | 0.0 |
| LOXIMVI | | | | | |
| Melanoma* SK- | 0.9 | 0.0 | Colon ca. SW480 | 1.6 | 0.0 |
| MEL-5 | | | | | |
| Squamous cell | 0.4 | 0.0 | Colon ca.* (SW480 | 5.3 | 0.1 |
| carcinoma SCC-4 | | | met) SW620 | | |
| Testis Pool | 2.5 | 2.0 | Colon ca. HT29 | 0.0 | 0.0 |
| Prostate ca.* | 3.6 | 0.0 | Colon ca. HCT-116 | 0.0 | 0.0 |
| (bone met) PC-3 | | | | | |
| Prostate Pool | 0.3 | 1.9 | Colon ca. CaCo-2 | 0.0 | 0.2 |
| Placenta | 3.0 | 1.3 | Colon cancer tissue | 1.8 | 0.1 |
| Uterus Pool | 1.3 | 0.1 | Colon ca. SW1116 | 0.0 | 0.0 |
| Ovarian ca. | 1.8 | 0.2 | Colon ca. Colo-205 | 0.0 | 0.0 |
| OVCAR-3 | | | | | |
| Ovarian ca. SK- | 2.4 | 0.7 | Colon ca. SW-48 | 0.2 | 0.0 |
| OV-3 | | | | | |
| Ovarian ca. | 0.8 | 0.9 | Colon Pool | 5.7 | 0.7 |
| OVCAR-4 | | | | | |
| Ovarian ca. | 1.5 | 1.8 | Small Intestine Pool | 22.4 | 0.8 |
| OVCAR-5 | | | | | |
| Ovarian ca. | 2.0 | 0.1 | Stomach Pool | 3.1 | 0.8 |
| IGROV-1 | | | | | |
| Ovarian ca. | 0.4 | 0.0 | Bone Marrow Pool | 3.8 | 0.3 |
| OVCAR-8 | | | | | |
| Ovary | 2.5 | 0.2 | Fetal Heart | 0.1 | 0.4 |
| Breast ca. MCF-7 | 0.5 | 0.1 | Heart Pool | 1.9 | 0.4 |
| Breast ca. MDA- | 1.0 | 0.0 | Lymph Node Pool | 14.6 | 0.8 |
| MB-231 | | | | | |
| Breast ca. BT 549 | 0.0 | 0.1 | Fetal Skeletal Muscle | 0.2 | 0.2 |
| Breast ca. T47D | 6.4 | 7.6 | Skeletal Muscle Pool | 0.4 | 0.2 |
| Breast ca. MDA-N | 0.0 | 0.0 | Spleen Pool | 6.2 | 0.2 |
| Breast Pool | 7.0 | 1.1 | Thymus pool | 4.1 | 1.0 |
| Trachea | 2.8 | 2.0 | CNS cancer | 0.0 | 0.0 |
| | | | (glio/astro) U87-MG | | |
| Lung | 0.3 | 0.0 | CNS cancer | 0.0 | 0.0 |
| | | | (glio/astro) U-118- | | |
| | | | MG | | |
| Fetal Lung | 7.5 | 17.0 | CNS cancer | 1.2 | 0.0 |
| | | | (neuro; met) SK-N- | | |
| | | | AS | | |
| Lung ca. NCI- | 0.0 | 0.0 | CNS cancer (astro) | 0.0 | 0.0 |
| N417 | | | SF-539 | | |
| Lung ca. LX-1 | 26.4 | 0.1 | CNS cancer (astro) | 0.0 | 0.0 |
| | | | SNB-75 | | |
| Lung ca. NCI- | 0.1 | 0.0 | CNS cancer (glio) | 0.9 | 0.0 |
| H146 | | | SNB-19 | | |
| Lung ca. SHP-77 | 100.0 | 0.1 | CNS cancer (glio) | 0.0 | 0.0 |
| | | | SF-295 | | |
| Lung ca. A549 | 0.0 | 0.0 | Brain (Amygdala) | 0.2 | 0.7 |
| | | | Pool | | |
| Lung ca. NCI- | 0.0 | 0.0 | Brain (cerebellum) | 0.6 | 0.9 |
| H526 | | | | | |
| Lung ca. NCI-H23 | 2.1 | 100.0 | Brain (fetal) | 5.9 | 0.9 |
| Lung ca. NCI- | 0.0 | 0.1 | Brain (Hippocampus) | 0.8 | 0.9 |
| H460 | | | Pool | | |
| Lung ca. HOP-62 | 0.0 | 0.0 | Cerebral Cortex Pool | 0.1 | 0.5 |
| Lung ca. NCI- | 3.3 | 0.6 | Brain (Substantia | 1.8 | 1.0 |
| H522 | | | nigra) Pool | | |
| Liver | 0.0 | 0.1 | Brain (Thalamus) | 1.5 | 0.9 |
| | | | Pool | | |
| Fetal Liver | 1.8 | 0.1 | Brain (whole) | 1.4 | 0.3 |
| Liver ca. HepG2 | 0.6 | 0.0 | Spinal Cord Pool | 3.2 | 5.9 |
| Kidney Pool | 25.7 | 0.5 | Adrenal Gland | 4.2 | 0.6 |
| Fetal Kidney | 5.4 | 2.8 | Pituitary gland Pool | 0.5 | 0.1 |
| Renal ca. 786-0 | 0.5 | 0.0 | Salivary Gland | 0.9 | 0.1 |
| Renal ca. A498 | 0.3 | 0.0 | Thyroid (female) | 0.4 | 0.2 |
| Renal ca. ACHN | 0.1 | 0.0 | Pancreatic ca. | 0.0 | 0.1 |
| | | | CAPAN2 | | |
| Renal ca. UO-31 | 1.3 | 0.0 | Pancreas Pool | 6.4 | 1.1 |

TABLE IE
Panel 4D

| | Rel. Exp. (%) | Rel. Exp. (%) | | Rel. Exp. (%) | Rel. Exp. (%) |
|---|---|---|---|---|---|
| | Ag3254, Run | Ag3339, Run | | Ag3254, Run | Ag3339, Run |
| Tissue Name | 164391364 | 165221778 | Tissue Name | 164391364 | 165221778 |
| Secondary Th1 act | 0.0 | 0.0 | HUVEC IL-1beta | 0.0 | 1.4 |
| Secondary Th2 act | 0.0 | 0.0 | HUVEC IFN gamma | 0.0 | 0.0 |
| Secondary Tr1 act | 0.0 | 0.0 | HUVEC TNF alpha + | 0.0 | 0.0 |
| | | | IFN gamma | | |
| Secondary Th1 rest | 0.0 | 0.0 | HUVEC TNF alpha + | 0.0 | 1.0 |
| | | | IL4 | | |
| Secondary Th2 rest | 0.0 | 0.0 | HUVEC IL-11 | 0.0 | 1.4 |
| Secondary Tr1 rest | 0.0 | 0.0 | Lung Microvascular EC | 0.0 | 0.0 |
| | | | none | | |
| Primary Th1 act | 0.0 | 0.0 | Lung Microvascular EC | 0.0 | 0.0 |
| | | | TNF alpha + IL-1beta | | |
| Primary Th2 act | 0.0 | 0.0 | Microvascular Dermal | 0.0 | 0.0 |
| | | | EC none | | |
| Primary Tr1 act | 0.0 | 0.0 | Microsvasular Dermal | 0.0 | 7.5 |
| | | | EC TNF alpha + IL- | | |
| | | | 1beta | | |
| Primary Th1 rest | 0.0 | 0.0 | Bronchial epithelium | 0.0 | 6.7 |
| | | | TNF alpha + IL1beta | | |
| Primary Th2 rest | 0.0 | 1.2 | Small airway epithelium | 0.0 | 0.0 |
| | | | none | | |
| Primary Tr1 rest | 0.0 | 0.9 | Small airway epithelium | 0.0 | 36.6 |
| | | | TNF alpha + IL-1beta | | |
| CD45RA CD4 | 0.0 | 0.0 | Coronery artery SMC | 0.0 | 0.0 |
| lymphocyte act | | | rest | | |
| CD45RO CD4 | 0.0 | 0.0 | Coronery artery SMC | 0.0 | 0.0 |
| lymphocyte act | | | TNF alpha + IL-1beta | | |
| CD8 lymphocyte act | 0.0 | 0.0 | Astrocytes rest | 0.0 | 0.0 |
| Secondary CD8 | 0.0 | 0.0 | Astrocytes TNF alpha + | 0.0 | 0.0 |
| lymphocyte rest | | | IL-1beta | | |
| Secondary CD8 | 0.0 | 0.0 | KU-812 (Basophil) rest | 0.0 | 1.9 |
| lymphocyte act | | | | | |
| CD4 lymphocyte none | 0.0 | 5.7 | KU-812 (Basophil) | 0.0 | 2.8 |
| | | | PMA/ionomycin | | |
| 2ry Th1/Th2/Tr1_anti- | 0.0 | 0.0 | CCD1106 | 0.0 | 0.0 |
| CD95 CH11 | | | (Keratinocytes) none | | |
| LAK cells rest | 0.0 | 1.1 | CCD1106 | 0.0 | 1.4 |
| | | | (Keratinocytes) | | |
| | | | TNF alpha + IL-1beta | | |
| LAK cells IL-2 | 0.0 | 0.0 | Liver cirrhosis | 0.0 | 4.6 |
| LAK cells IL-2 + IL-12 | 0.0 | 0.0 | Lupus kidney | 0.0 | 32.1 |
| LAK cells IL-2 + IFN | 0.0 | 0.0 | NCI-H292 none | 0.0 | 0.0 |
| gamma | | | | | |
| LAK cells IL-2 + IL- | 0.0 | 0.7 | NCI-H292 IL-4 | 0.0 | 1.3 |
| 18 | | | | | |
| LAK cells | 0.0 | 1.2 | NCI-H292 IL-9 | 0.0 | 2.5 |
| PMA/ionomycin | | | | | |
| NK Cells IL-2 rest | 0.0 | 3.0 | NCI-H292 IL-13 | 0.0 | 0.0 |
| Two Way MLR 3 day | 0.0 | 2.7 | NCI-H292 IFN gamma | 0.0 | 0.0 |
| Two Way MLR 5 day | 0.0 | 0.0 | HPAEC none | 0.0 | 0.0 |
| Two Way MLR 7 day | 100.0 | 0.0 | HPAEC TNF alpha + | 0.0 | 44.8 |
| | | | IL-1beta | | |
| PBMC rest | 0.0 | 16.8 | Lung fibroblast none | 0.0 | 0.0 |
| PBMC PWM | 0.0 | 2.4 | Lung fibroblast TNF | 0.0 | 0.0 |
| | | | alpha + IL-1beta | | |
| PBMC PHA-L | 0.0 | 0.0 | Lung fibroblast IL-4 | 0.0 | 0.0 |
| Ramos (B cell) none | 0.0 | 0.0 | Lung fibroblast IL-9 | 0.0 | 0.0 |
| Ramos (B cell) | 0.0 | 0.0 | Lung fibroblast IL-13 | 0.0 | 0.0 |
| ionomycin | | | | | |
| B lymphocytes PWM | 0.0 | 0.0 | Lung fibroblast IFN | 0.0 | 0.0 |
| | | | gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 1.5 |
| | | | CCD1070 TNF alpha | | |
| EOL-1 dbcAMP | 0.0 | 1.4 | Dermal fibroblast | 0.0 | 0.0 |
| PMA/ionomycin | | | CCD1070 IL-1beta | | |
| Dendritic cells none | 0.0 | 1.3 | Dermal fibroblast IFN | 0.0 | 0.0 |
| | | | gamma | | |
| Dendritic cells LPS | 0.0 | 0.0 | Dermal fibroblast IL-4 | 0.0 | 0.0 |
| Dendritic cells anti- | 0.0 | 0.0 | IBD Colitis 2 | 0.0 | 0.5 |
| CD40 | | | | | |
| Monocytes rest | 0.0 | 49.3 | IBD Crohn's | 0.0 | 0.0 |
| Monocytes LPS | 0.0 | 79.0 | Colon | 0.0 | 15.1 |
| Macrophages rest | 0.0 | 9.0 | Lung | 0.0 | 100.0 |
| Macrophages LPS | 0.0 | 3.1 | Thymus | 0.0 | 95.3 |
| HUVEC none | 0.0 | 0.0 | Kidney | 0.0 | 32.3 |
| HUVEC starved | 0.0 | 0.0 | | | |

CNS_neurodegeneration_v1.0 Summary: Ag3339 This panel confirms the expression of the CG57488-01 gene at low levels in the brain in an independent group of individuals. Interestingly, this gene appears to be slightly upregulated in the temporal cortex of Alzheimer's disease patients. Therefore, therapeutic modulation of the activity of this gene or its protein product may be of use to decrease neuronal death in the treatment of Alzheimer's disease. Ag23254 Expression of this gene is low/undetectable (CTs>35) across all of the samples on this panel (data not shown).

General_screening_panel_v1.4 Summary: Ag3254 Expression of the CG57488-01 gene is highest in lung cancer cell line SHP-77 (CT=29.1). Expression of this gene is also higher in fetal lung (CT=32.9) than in adult lung (CT=37.7), suggesting that expression of this gene can be used to distinguish fetal lung from adult lung. In addition, significant expression of this gene is seen in adult and fetal kidney. This gene resembles members of the alpha-2-macroglobulin family. It has been shown that patients with chronic renal failure treated with haemodialysis have significantly lower blood serum levels of alpha-2-macroglobulin than healthy patients (ref. 1). Therefore, therapeutic modulation of the activity of this gene or its protein product, using small molecule drugs, antibodies, or protein therapeutics, may be used as a treatment for patients with renal failure.

Among tissues with metabolic or endocrine function, this gene is expressed at low levels in adrenal gland, heart, and pancreas. Therefore, therapeutic modulation of the activity of this gene or its protein product may prove useful in the treatment of endocrine/metabolically related diseases, such as obesity and diabetes.

The CG57488-01 gene is expressed at low levels in some regions of the CNS, including fetal brain, substanitia nigra and spinal cord. Alpha-2-macroglobulin, a serum pan-protease inhibitor, has been implicated in Alzheimer's disease based on its ability to mediate the clearance and degradation of A-beta, the major component of amyloid beta deposits (ref. 2). Therefore, the CG57488-01 gene may also play a role in the development of Alzheimer's disease.

References:

1. Bartelik S, Starzyk J, Krajewska R. Concentration of prealbumin, ceruloplasmin, alpha-macroglobulin and haptoglobin in blood serum of patients with chronic non-A, non-B hepatitis treated with hemodialysis for chronic renal failure. Wiad Lek 1992 October;45(19-20):733-6

In 25 patients with chronic renal failure, treated with haemodialysis (13 patients with chronic non-A, non-B hepatitis, and 12 cases without evidence of hepatocellular damage), and in 20 healthy persons, blood serum concentrations were determined of prealbumin, ceruloplasmin, alpha 2-macroglobulin, and haptoglobin. It was found that the concentrations of these proteins in both subgroups of patients were not significantly different. The concentration of prealbumin was higher, and that of alpha 2-macroglobulin and haptoglobin was significantly lower in comparison with healthy subjects.

PMID: 1284260

2. Blacker D, Wilcox M A, Laird N M, Rodes L, Horvath S M, Go R C, Perry R, Watson B Jr, Bassett S S, McInnis M G, Albert M S, Hyman B T, Tanzi R E. Alpha-2 macroglobulin is genetically associated with Alzheimer disease. Nat Genet 1998 August;19(4):357-60

Alpha-2-macroglobulin (alpha-2M; encoded by the gene A2M) is a serum pan-protease inhibitor that has been implicated in Alzheimer disease (AD) based on its ability to mediate the clearance and degradation of A beta, the major component of beta-amyloid deposits. Analysis of a deletion in the A2M gene at the 5′ splice site of ‘exon II’ of the bait region (exon 18) revealed that inheritance of the deletion (A2M-2) confers increased risk for AD (Mantel Haenzel odds ratio=3.56, P=0.001). The sibship disequilibrium test (SDT) also revealed a significant association between A2M and AD (P=0.00009). These values were comparable to those obtained for the APOE-epsilon4 allele in the same sample, but in contrast to APOE epsilon4, A2M-2 did not affect age of onset. The observed association of A2M with AD did not appear to account for the previously published linkage of AD to chromosome 12, which we were unable to confirm in this sample. A2M, LR(encoding the alpha-2M receptor) and the genes for two other LRP ligands, APOE and APP (encoding the amyloid beta protein precursor), have now all been genetically linked to AD, suggesting that these proteins may participate in a common neuropathogenic pathway leading to AD.

PMID: 9697696

Panel 4D Summary: Ag3339 Expression of the CG57488-01 gene is highest in normal lung and thymus (CTs=31). In addition, expression of this gene is upregulated in small airway epithelium treated with TNFalpha and IL-1 beta. Therefore, therapeutic modulation of the activity of this gene or its protein product, using small molecule drugs, antibodies or protein therapeutics, may be of benefit in the treatment of asthma and emphysema. Furthermore, this gene is moderately expressed in monocytes, suggesting that this gene may also play a role in rheumatoid arthritis or other autoimmune diseases. Ag3254 Results from one experiment with the CG57488-01 gene are not included. The amp plot indicates that there were experimental difficulties with this run.

NOV12

Expression of NOV12A/CG57526-01 and NOV12B/CG57526-02 was assessed using the primer-probe set Ag3275, described in Table JA. Results of the RTQ-PCR runs are shown in Tables JB, JC and JD.

TABLE JA
Probe Name Ag3275

| Primers | Sequences | Length | Start Position | SEQ ID NO |
|---|---|---|---|---|
| Forward | 5′-ggaactactggctggagatttt-3′ | 22 | 1500 | 285 |
| Probe | TET-5′-ctgaacctgctcatgttggcctttct-3′-TAMRA | 26 | 1541 | 286 |
| Reverse | 5′-aaacataaacgacacccacaac-3′ | 22 | 1571 | 287 |

TABLE JB
CNS_neurodegeneration_v1.0

| | Rel. Exp. (%) | | Rel. Exp. (%) |
|---|---|---|---|
| | Ag3275, Run | | Ag3275, Run |
| Tissue Name | 210059235 | Tissue Name | 210059235 |
| AD 1 Hippo | 0.0 | Control (Path) 3 Temporal | 0.0 |
| | | Ctx | |
| AD 2 Hippo | 0.0 | Control (Path) 4 Temporal | 0.0 |
| | | Ctx | |
| AD 3 Hippo | 0.0 | AD 1 Occipital Ctx | 0.0 |
| AD 4 Hippo | 0.0 | AD 2 Occipital Ctx | 0.0 |
| | | (Missing) | |
| AD 5 Hippo | 0.0 | AD 3 Occipital Ctx | 0.0 |
| AD 6 Hippo | 22.5 | AD 4 Occipital Ctx | 0.0 |
| Control 2 Hippo | 59.0 | AD 5 Occipital Ctx | 0.0 |
| Control 4 Hippo | 0.0 | AD 6 Occipital Ctx | 0.0 |
| Control (Path) 3 Hippo | 0.0 | Control 1 Occipital Ctx | 0.0 |
| AD 1 Temporal Ctx | 0.0 | Control 2 Occipital Ctx | 0.0 |
| AD 2 Temporal Ctx | 0.0 | Control 3 Occipital Ctx | 0.0 |
| AD 3 Temporal Ctx | 26.8 | Control 4 Occipital Ctx | 51.8 |
| AD 4 Temporal Ctx | 0.0 | Control (Path) 1 Occipital | 100.0 |
| | | Ctx | |
| AD 5 Inf Temporal Ctx | 0.0 | Control (Path) 2 Occipital | 27.7 |
| | | Ctx | |
| AD 5 Sup Temporal Ctx | 0.0 | Control (Path) 3 Occipital | 0.0 |
| | | Ctx | |
| AD 6 Inf Temporal Ctx | 0.0 | Control (Path) 4 Occipital | 0.0 |
| | | Ctx | |
| AD 6 Sup Temporal Ctx | 0.0 | Control 1 Parietal Ctx | 0.0 |
| Control 1 Temporal Ctx | 96.6 | Control 2 Parietal Ctx | 0.0 |
| Control 2 Temporal Ctx | 62.9 | Control 3 Parietal Ctx | 48.0 |
| Control 3 Temporal Ctx | 0.0 | Control (Path) 1 Parietal | 0.0 |
| | | Ctx | |
| Control 3 Temporal Ctx | 0.0 | Control (Path) 2 Parietal | 19.5 |
| | | Ctx | |
| Control (Path) 1 Temporal | 73.2 | Control (Path) 3 Parietal | 0.0 |
| Ctx | | Ctx | |
| Control (Path) 2 Temporal | 0.0 | Control (Path) 4 Parietal | 0.0 |
| Ctx | | Ctx | |

TABLE JC
General_screening_panel_v1.4

| | Rel. Exp. (%) | | Rel. Exp. (%) |
|---|---|---|---|
| | Ag3275, Run | | Ag3275, Run |
| Tissue Name | 215775538 | Tissue Name | 215775538 |
| Adipose | 0.0 | Renal ca. TK-10 | 5.1 |
| Melanoma* Hs688(A).T | 0.0 | Bladder | 0.0 |
| Melanoma* Hs688(B).T | 5.5 | Gastric ca. (liver met.) NCI- | 9.4 |
| | | N87 | |
| Melanoma* M14 | 0.0 | Gastric ca. KATO III | 5.1 |
| Melanoma* LOXIMVI | 0.0 | Colon ca. SW-948 | 3.7 |
| Melanoma* SK-MEL-5 | 0.0 | Colon ca. SW480 | 0.0 |
| Squamous cell carcinoma | 4.4 | Colon ca* (SW480 met) | 4.0 |
| SCC-4 | | SW620 | |
| Test is Pool | 0.0 | Colon ca. HT29 | 4.2 |
| Prostate ca * (bone met) | 0.0 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 17.2 |
| Ovarian ca. OVCAR-4 | 0.0 | Colon Pool | 3.2 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 10.4 |
| Breast ca. MDA-MB-231 | 10.4 | Lymph Node Pool | 4.8 |
| Breast ca. BT 549 | 0.0 | Fetal Skeletal Muscle | 0.0 |
| Breast ca. T47D | 0.0 | Skeletal Muscle Pool | 10.5 |
| Breast ca. MDA-N | 0.0 | Spleen Pool | 4.6 |
| Breast Pool | 0.0 | Thymus Pool | 5.0 |
| Trachea | 0.0 | CNS cancer (glio/astro) U87- | 5.0 |
| | | MG | |
| Lung | 0.0 | CNS cancer (glio/astro) U- | 0.0 |
| | | 118-MG | |
| Fetal Lung | 0.0 | CNS cancer (neuro; met) SK- | 61.6 |
| | | N-AS | |
| Lung ca. NCI-N417 | 0.0 | CNS cancer (astro) SF-539 | 0.0 |
| Lung ca. LX-1 | 6.7 | CNS cancer (astro) SNB-75 | 0.0 |
| Lung ca. NCI-H146 | 7.5 | CNS cancer (glio) SNB-19 | 0.0 |
| Lung ca. SHP-77 | 59.0 | CNS cancer (glio) SF-295 | 0.0 |
| Lung ca. A549 | 4.4 | Brain (Amygdala) Pool | 0.0 |
| Lung ca. NCI-H526 | 10.2 | Brain (cerebellum) | 0.0 |
| Lung ca. NCI-H23 | 100.0 | Brain (fetal) | 5.3 |
| Lung ca. NCI-H460 | 7.6 | Brain (Hippocampus) Pool | 0.0 |
| Lung ca HOP-62 | 0.0 | Cerebral Cortex Pool | 0.0 |
| Lung ca. NCI-H522 | 5.6 | Brain (Substantia nigra) Pool | 1.3 |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 2.4 |
| Fetal Kidney | 55.5 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 9.4 | Pancreatic ca. CAPAN2 | 0.0 |
| Renal ca. UO-31 | 0.0 | Pancreas Pool | 0.0 |

TABLE JD
Panel 4D

| | Rel. Exp. (%) | | Rel. Exp. (%) |
|---|---|---|---|
| | Ag3275, Run | | Ag3275, Run |
| Tissue Name | 164634885 | Tissue Name | 164634885 |
| Secondary Th1 act | 0.7 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.8 | Lung Microvascular EC | 0.0 |
| | | TNFalpha + IL-1beta | |
| Primary Th2 act | 0.5 | Microvascular Dermal EC none | 0.0 |
| Primary Tr1 act | 0.6 | Microsvasular Dermal EC | 0.0 |
| | | TNFalpha + IL-1beta | |
| Primary Th1 rest | 0.0 | Bronchial epithelium TNFalpha + | 0.0 |
| | | IL1beta | |
| Primary Th2 rest | 0.7 | Small airway epithelium none | 0.0 |
| Primary Tr1 rest | 0.0 | Small airway epithelium | 0.0 |
| | | TNFalpha + IL-1beta | |
| CD45RA CD4 lymphocyte | 0.0 | Coronery artery SMC rest | 0.0 |
| act | | | |
| CD45RO CD4 lymphocyte | 1.3 | Coronery artery SMC TNFalpha + | 0.0 |
| act | | IL-1beta | |
| CD8 lymphocyte act | 0.0 | Astrocytes rest | 0.0 |
| Secondary CD8 lymphocyte | 0.0 | Astrocytes TNFalpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNFalpha + IL-1beta | |
| LAK cells IL-2 | 0.0 | Liver cirrhosis | 4.5 |
| LAK cells IL-2 + IL-12 | 0.0 | Lupus kidney | 3.6 |
| LAK cells IL-2 + IFN gamma | 0.0 | NCI-H292 none | 3.6 |
| LAK cells IL-2 + IL-18 | 0.0 | NCI-H292 IL-4 | 9.5 |
| LAK cells PMA/ionomycin | 0.0 | NCI-H292 IL-9 | 1.7 |
| NK Cells IL-2 rest | 0.0 | NCI-H292 IL-13 | 3.6 |
| Two Way MLR 3 day | 0.0 | NCI-H292 IFN gamma | 0.0 |
| Two Way MLR 5 day | 0.3 | HPAEC none | 1.4 |
| Two Way MLR 7 day | 0.9 | HPAEC TNF alpha + IL-1 beta | 0.7 |
| PBMC rest | 0.0 | Lung fibroblast none | 0.0 |
| PBMC PWM | 0.0 | Lung fibroblast TNF alpha + IL-1 | 0.0 |
| | | beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 rest | 0.0 |
| IL-4 | | | |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL-1 | 0.0 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 10.9 |
| Macrophages rest | 0.0 | Lung | 2.5 |
| Macrophages LPS | 0.0 | Thymus | 100.0 |
| HUVEC none | 0.0 | Kidney | 1.4 |
| HUVEC starved | 0.0 | | |

CNS_neurodegeneration_v1.0 Summary: Ag3275 Expression of this gene is low/undetectable (CTs>35) across all of the samples on this panel (data not shown).

General_screening_panel_v1.4 Summary: Ag3275 Expression of the CG57526-01 gene is highest in lung cancer cell line NCI-H23 (CT=32.8). Expression of this gene is higher in several additional lung cancer cell lines when compared to normal lung. Thus, expression of this gene may be used as a marker for lung cancer. Furthermore, therapeutic modulation of the activity of this gene or its protein product, using small molecule drugs, antibodies or protein therapeutics, may be of use in the treatment of lung cancer. The CG57526-01 gene encodes a protein with homology to sodium- and chloride-dependent transporter XT3, a rotein that mediates transit of structurally related small hydrophilic substances across plasma membranes (ref. 1).

Interestingly, while there is low expression of this gene in fetal kidney (CT=33.6), this gene does not seem to be expressed at detectable levels in adult kidney. This observation suggests that expression of this gene can be used to distinguish fetal from adult kidney. Expression of this gene is consistent with what is known about the XT3 transporter (ref. 1).

References:

1. Nash S R, Giros B, Kingsmore S F, Kim K M, el-Mestikawy S, Dong Q, Flumagalli F, Seldin M F, Caron M G. Cloning, gene structure and genomic localization of an orphan transporter from mouse kidney with six alternatively-spliced isoforms. Receptors Channels 1998;6(2):113-28

Two genes were identified and characterized that express cDNAs related to previously identified neurotransmitter and/or osmolyte transporters, but which are expressed specifically in the kidney. RNA transcribed from one of these two genes (XT2) was found to undergo an extensive degree of alternative splicing to generate six distinct isoforms. The intron-exon structure of the XT2 gene and the sites of alternative splicing were identified. Expression of the second gene (XT3) was found to be conserved in human kidney, and partial sequence was obtained from a human cDNA library. The expressions of both XT2 and XT3 RNAs were determined in mouse and human tissues, respectively, and the locations of the two genes within the mouse genome were identified. Screening experiments to identify the substrate(s) of these proteins failed to identify specific uptake with any of the tested compounds; however, immunofluorescent microscopy demonstrated that epitope-tagged variants of the protein products of the XT2 and XT3 cDNAs were present on the plasma membrane of transfected cells.

PMID: 9932288

Panel 4D Summary: Ag3275 Expression of the CG57526-01 gene is highest in thymus (CT=29). Therefore, expression of this gene may be used to distinguish thymus from the other samples on this panel. In addition, the putative ion transporter encoded for by the CG57526-01 gene could play an important role in T cell development. Small molecule therapeutics or antibody therapeutics designed against the protein encoded for by this gene could be utilized to modulate immune function (T cell development) and be important for organ transplant, AIDS treatment or post chemotherapy immune reconstitiution. In addition, this gene is expressed at low levels in the NCI-H292 mucoepidermoid cell line, consistent with its expression in lung cancer cell lines on General_screening_panel_v1.4.

NOV13

Expression of NOV13/CG57570-01 was assessed using the primer-probe set Ag3288, described in Table KA. Results of the RTQ-PCR runs are shown in Tables KB, KC and KD.

TABLE KA
Probe Name Ag3288

| Primers | Sequences | Length | Start Position | SEQ ID NO |
|---|---|---|---|---|
| Forward | 5′-tctacaccatcagctgtatgca-3′ | 22 | 1380 | 288 |
| Probe | TET-5′-caccaccctcacactcatcttcatca-3′-TAMRA | 26 | 1411 | 289 |
| Reverse | 5′-gcagtgcagctgtcatatagaa-3′ | 22 | 1439 | 290 |

TABLE KB
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3288, Run | Ag3288, Run | | Ag3288, Run | Ag3288, Run |
| Tissue Name | 210058660 | 229929907 | Tissue Name | 210058660 | 229929907 |
| AD 1 Hippo | 26.6 | 31.0 | Control (Path) 3 | 13.3 | 15.1 |
| | | | Temporal Ctx | | |
| AD 2 Hippo | 43.5 | 48.0 | Control (Path) 4 | 33.7 | 36.3 |
| | | | Temporal Ctx | | |
| AD 3 Hippo | 15.0 | 14.8 | AD 1 Occipital | 25.7 | 15.5 |
| | | | Ctx | | |
| AD 4 Hippo | 13.0 | 11.5 | AD 2 Occipital | 0.0 | 0.0 |
| | | | Ctx (Missing) | | |
| AD 5 hippo | 97.3 | 100.0 | AD 3 Occipital | 16.5 | 15.8 |
| | | | Ctx | | |
| AD 6 Hippo | 100.0 | 90.1 | AD 4 Occipital | 26.1 | 35.1 |
| | | | Ctx | | |
| Control 2 Hippo | 29.3 | 28.9 | AD 5 Occipital | 26.4 | 28.5 |
| | | | Ctx | | |
| Control 4 Hippo | 33.7 | 41.2 | AD 6 Occipital | 33.2 | 37.6 |
| | | | Ctx | | |
| Control (Path) 3 | 22.8 | 27.9 | Control 1 | 8.8 | 10.4 |
| Hippo | | | Occipital Ctx | | |
| AD 1 Temporal | 34.2 | 35.8 | Control 2 | 51.8 | 59.9 |
| Ctx | | | Occipital Ctx | | |
| AD 2 Temporal | 47.3 | 52.1 | Control 3 | 28.5 | 12.8 |
| Ctx | | | Occipital Ctx | | |
| AD 3 Temporal | 14.0 | 18.6 | Control 4 | 16.5 | 17.8 |
| Ctx | | | Occipital Ctx | | |
| AD 4 Temporal | 34.6 | 46.7 | Control (Path) 1 | 66.0 | 82.4 |
| Ctx | | | Occipital Ctx | | |
| AD 5 Inf Temporal | 80.1 | 100.0 | Control (Path) 2 | 11.3 | 14.4 |
| Ctx | | | Occipital Ctx | | |
| AD 5 | 64.2 | 85.9 | Control (Path) 3 | 9.5 | 8.5 |
| SupTemporal Ctx | | | Occipital Ctx | | |
| AD 6 Inf Temporal | 68.8 | 87.1 | Control (Path) 4 | 13.9 | 17.7 |
| Ctx | | | Occipital Ctx | | |
| AD 6 Sup | 66.4 | 87.1 | Control 1 | 11.1 | 15.8 |
| Temporal Ctx | | | Parietal Ctx | | |
| Control 1 | 11.3 | 16.2 | Control 2 | 48.0 | 66.0 |
| Temporal Ctx | | | Parietal Ctx | | |
| Control 2 | 33.0 | 36.3 | Control 3 | 17.0 | 22.2 |
| Temporal Ctx | | | Parietal Ctx | | |
| Control 3 | 19.2 | 18.9 | Control (Path) 1 | 55.5 | 66.4 |
| Temporal Ctx | | | Parietal Ctx | | |
| Control 4 | 14.7 | 17.8 | Control (Path) 2 | 30.6 | 34.2 |
| Temporal Ctx | | | Parietal Ctx | | |
| Control (Path) 1 | 32.3 | 50.7 | Control (Path) 3 | 14.3 | 11.3 |
| Temporal Ctx | | | Parietal Ctx | | |
| Control (Path) 2 | 36.9 | 13.2 | Control (Path) 4 | 37.1 | 43.2 |
| Temporal Ctx | | | Parietal Ctx | | |

TABLE KC
General_screening_panel_v1.4

| | Rel. Exp. (%) | | Rel. Exp. (%) |
|---|---|---|---|
| | Ag3288, Run | | Ag3288, Run |
| Tissue Name | 216516909 | Tissue Name | 216516909 |
| Adipose | 15.8 | Renal ca. TK-10 | 24.0 |
| Melanoma* Hs688(A).T | 38.4 | Bladder | 26.8 |
| Melanoma* Hs688(B).T | 51.4 | Gastric ca. (liver met.) NCI- | 55.1 |
| | | N87 | |
| Melanoma* M14 | 43.5 | Gastric ca. KATO III | 53.6 |
| Melanoma* LOXIMVI | 25.0 | Colon ca. SW-948 | 19.1 |
| Melanoma* SK-MEL-5 | 52.9 | Colon ca. SW480 | 41.2 |
| Squamous cell carcinoma | 16.5 | Colon ca.* (SW480 met) | 14.4 |
| SCC-4 | | SW620 | |
| Testis Pool | 44.4 | Colon ca. HT29 | 9.5 |
| Prostate ca.* (bone met) | 57.4 | Colon ca. HCT-116 | 50.7 |
| PC-3 | | | |
| Prostate Pool | 16.2 | Colon ca. CaCo-2 | 9.5 |
| Placenta | 4.3 | Colon cancer tissue | 7.3 |
| Uterus Pool | 4.9 | Colon ca. SW1116 | 6.7 |
| Ovarian ca. OVCAR-3 | 37.9 | Colon ca. Colo-205 | 10.5 |
| Ovarian ca. SK-OV-3 | 39.0 | Colon ca. SW-48 | 7.3 |
| Ovarian ca. OVCAR-4 | 18.0 | Colon Pool | 15.9 |
| Ovarian ca. OVCAR-5 | 63.3 | Small Intestine Pool | 15.0 |
| Ovarian ca. IGROV-1 | 14.5 | Stomach Pool | 9.6 |
| Ovarian ca. OVCAR-8 | 12.1 | Bone Marrow Pool | 9.4 |
| Ovary | 17.2 | Fetal Heart | 72.7 |
| Breast ca. MCF-7 | 32.5 | Heart Pool | 66.9 |
| Breast ca. MDA-MB-231 | 47.3 | Lymph Node Pool | 19.3 |
| Breast ca. BT 549 | 58.2 | Fetal Skeletal Muscle | 17.6 |
| Breast ca. T47D | 73.7 | Skeletal Muscle Pool | 63.7 |
| Breast ca. MDA-N | 12.6 | Spleen Pool | 11.0 |
| Breast Pool | 14.6 | Thymus Pool | 7.5 |
| Trachea | 14.6 | CNS cancer (glio/astro) U87- | 30.8 |
| | | MG | |
| Lung | 7.5 | CNS cancer (glio/astro) U- | 44.1 |
| | | 118-MG | |
| Fetal Lung | 63.7 | CNS cancer (neuro; met) SK- | 37.9 |
| | | N-AS | |
| Lung ca. NCI-N417 | 2.7 | CNS cancer (astro) SF-539 | 33.2 |
| Lung ca. LX-1 | 19.5 | CNS cancer (astro) SNB-75 | 34.6 |
| Lung ca. NCI-H146 | 4.9 | CNS cancer (glio) SNB-19 | 18.9 |
| Lung ca. SHP-77 | 12.0 | CNS cancer (glio) SF-295 | 64.2 |
| Lung ca. A549 | 24.8 | Brain (Amygdala) Pool | 13.4 |
| Lung ca. NCI-H526 | 8.8 | Brain (cerebellum) | 100.0 |
| Lung ca. NCI-H23 | 23.8 | Brain (fetal) | 28.5 |
| Lung ca. NCI-H460 | 29.5 | Brain (Hippocampus) Pool | 21.5 |
| Lung ca. HOP-62 | 23.0 | Cerebral Cortex Pool | 22.8 |
| Lung ca. NCI-H522 | 36.1 | Brain (Substantia nigra) Pool | 19.2 |
| Liver | 0.5 | Brain (Thalamus) Pool | 26.8 |
| Fetal Liver | 27.2 | Brain (whole) | 40.9 |
| Liver ca. HepG2 | 0.2 | Spinal Cord Pool | 19.8 |
| Kidney Pool | 23.0 | Adrenal Gland | 40.3 |
| Fetal Kidney | 21.2 | Pituitary gland Pool | 5.8 |
| Renal ca. 786-0 | 32.1 | Salivary Gland | 6.4 |
| Renal ca. A498 | 17.6 | Thyroid (female) | 17.2 |
| Renal ca. ACHN | 36.1 | Pancreatic ca. CAPAN2 | 14.8 |
| Renal ca. UO-31 | 21.0 | Pancreas Pool | 22.8 |

TABLE KD
Panel 4D

| | Rel. Exp. (%) | | Rel. Exp. (%) |
|---|---|---|---|
| | Ag3288, Run | | Ag3288, Run |
| Tissue Name | 165007638 | Tissue Name | 165007638 |
| Secondary Th1 act | 15.0 | HUVEC IL-1beta | 10.7 |
| Secondary Th2 act | 15.8 | HUVEC IFN gamma | 9.3 |
| Secondary Tr1 act | 15.0 | HUVEC TNF alpha + IFN gamma | 20.9 |
| Secondary Th1 rest | 3.0 | HUVEC TNF alpha + IL4 | 14.9 |
| Secondary Th2 rest | 8.5 | HUVEC IL-11 | 6.6 |
| Secondary Tr1 rest | 4.4 | Lung Microvascular EC none | 9.3 |
| Primary Th1 act | 28.5 | Lung Microvascular EC | 58.2 |
| | | TNFalpha + IL-1beta | |
| Primary Th2 act | 19.5 | Microvascular Dermal EC none | 22.4 |
| Primary Tr1 act | 24.0 | Microsvasular Dermal EC | 63.3 |
| | | TNFalpha + IL-1beta | |
| Primary Th1 rest | 11.5 | Bronchial epithelium | 20.6 |
| | | TNFalpha + IL1beta | |
| Primary Th2 rest | 8.5 | Small airway epithelium none | 3.8 |
| Primary Tr1 rest | 4.6 | Small airway epithelium | 48.6 |
| | | TNFalpha + IL-1beta | |
| CD45RA CD4 lymphocyte | 12.2 | Coronery artery SMC rest | 11.7 |
| act | | | |
| CD45RO CD4 lymphocyte | 21.2 | Coronery artery SMC TNFalpha + | 9.1 |
| act | | IL-1beta | |
| CD8 lymphocyte act | 14.2 | Astrocytes rest | 16.4 |
| Secondary CD8 lymphocyte | 22.1 | Astrocytes TNFalpha + IL-1beta | 12.9 |
| rest | | | |
| Secondary CD8 lymphocyte | 10.8 | KU-812 (Basophil) rest | 16.8 |
| act | | | |
| CD4 lymphocyte none | 5.1 | KU-812 (Basophil) | 50.3 |
| | | PMA/ionomycin | |
| 2ry Th1/Th2/Tr1_anti-CD95 | 4.0 | CCD1106 (Keratinocytes) none | 14.0 |
| CH11 | | | |
| LAK cells rest | 4.8 | CCD1106 (Keratinocytes) | 10.8 |
| | | TNFalpha + IL-1beta | |
| LAK cells IL-2 | 10.6 | Liver cirrhosis | 3.3 |
| LAK cells IL-2 + IL-12 | 15.7 | Lupus kidney | 1.6 |
| LAK cells IL-2 + IFN gamma | 19.3 | NCI-H292 none | 14.6 |
| LAK cells IL-2 + IL-18 | 14.2 | NCI-H292 IL-4 | 18.9 |
| LAK cells PMA/ionomycin | 5.7 | NCI-H292 IL-9 | 16.3 |
| NK Cells IL-2 rest | 7.9 | NCI-H292 IL-13 | 13.1 |
| Two Way MLR 3 day | 11.5 | NCI-H292 IFN gamma | 11.2 |
| Two Way MLR 5 day | 9.7 | HPAEC none | 11.5 |
| Two Way MLR 7 day | 6.7 | HPAEC TNF alpha + IL-1 beta | 31.0 |
| PBMC rest | 3.6 | Lung fibroblast none | 13.9 |
| PBMC PWM | 61.6 | Lung fibroblast TNF alpha + IL-1 | 13.6 |
| | | beta | |
| PBMC PHA-L | 31.6 | Lung fibroblast IL-4 | 28.1 |
| Ramos (B cell) none | 13.7 | Lung fibroblast IL-9 | 22.2 |
| Ramos (B cell) ionomycin | 94.6 | Lung fibroblast IL-13 | 13.5 |
| B lymphocytes PWM | 63.7 | Lung fibroblast IFN gamma | 23.7 |
| B lymphocytes CD40L and | 100.0 | Dermal fibroblast CCD1070 rest | 30.4 |
| IL-4 | | | |
| EOL-1 dbcAMP | 3.9 | Dermal fibroblast CCD1070 TNF | 49.0 |
| | | alpha | |
| EOL-1 dbcAMP | 6.7 | Dermal fibroblast CCD1070 IL-1 | 15.6 |
| PMA/ionomycin | | beta | |
| Dendritic cells none | 2.2 | Dermal fibroblast IFN gamma | 6.4 |
| Dendritic cells LPS | 1.9 | Dermal fibroblast IL-4 | 14.2 |
| Dendritic cells anti-CD40 | 0.8 | IBD Colitis 2 | 1.6 |
| Monocytes rest | 0.5 | IBD Crohn's | 0.9 |
| Monocytes LPS | 5.8 | Colon | 5.9 |
| Macrophages rest | 1.0 | Lung | 13.8 |
| Macrophages LPS | 7.2 | Thymus | 48.0 |
| HUVEC none | 10.6 | Kidney | 20.4 |
| HUVEC starved | 17.3 | | |

CNS_neurodegeneration_v1.0 Summary: Ag3288 Results from two experiments using the same probe/primer set are in excellent agreement. The CG57570-01 gene was found to upregulated in the temporal cortex of Alzheimer's disease (AD) patients by two separate TaqMan runs (p=0.0007 when analyzed by ANCOVA); the temporal cortex shows marked neuronal loss in the early to middle stages of AD. Upregulation of CG57570-01 gene expression, however, was not apparent in the occipital cortex, where neuronal degeneration does not occur in AD. Taken together, these data suggest that the protein in question is involved in the pathologic process of Alzheimer's disease, making this an excellent small molecule drug target.

The CG57570-01 gene encodes a protein with homology to cation transporters. For example, iron transporters in the brain have been shown to play an important role in age-related neurodegenerative diseases, including Parkinson's disease, Alzheimer's disease, Huntington's disease and amyotrophic lateral sclerosis (ref. 1).

References:

1. Qian Z M, Wang Q. Expression of iron transport proteins and excessive iron accumulation in the brain in neurodegenerative disorders. Brain Res Brain Res Rev 1998 August;27(3):257-67

New findings on the role of LfR (lactotransferrin receptor), MTf (melanotransferrin), CP (ceruloplasmin) and DCT1 (Divalent Cation Transporter) in brain iron transport, obtained during the past 3 years, are important advances in the fields of physiology and pathophysiology of brain iron metabolism. According to these findings, disruption in the expression of these proteins in the brain is probably one of the important causes of the altered brain iron metabolism in age-related neurodegenerative diseases, including Parkinson's Disease, Alzheimer's disease, Huntington's disease and amyotrophic lateral sclerosis. Further studies on the involvement of LtR, MTf and DCT1 in iron uptake by and CP in iron egress from different types of brain cells as well as control mechanisms of expression of these proteins in the brain are critical for elucidating the causes of excessive accumulation of iron in the brain and neuronial death in neurodegenerative diseases.

PMID: 9729418

General_screening_panel_v1.4 Summary: Ag3288 The CG57570-01 gene is expressed at high to moderate levels across almost all samples in this panel, with highest expression in the cerebellum (CT=26.7). This gene is also moderately expressed in all other regions of the CNS examined, including in amygdala, substantia nigra, thalamus, cerebral cortex, and spinal cord, suggesting that this gene may play a role in central nervous system disorders such as Alzheimer's disease, Parkinson's disease, epilepsy, multiple sclerosis, schizophrenia and depression.

This gene is also expressed in a number of tissues with metabolic or endocrine function, including adipose, adrenal gland, gastrointestinal tract, pancreas, skeletal muscle and thyroid. Therefore, therapeutic modulation of the activity of this gene may prove useful in the treatment of endocrine/metabolically related diseases, such as obesity and diabetes. Interestingly, this gene is differentially expressed in adult liver (CT=34) vs fetal liver (CT=29), suggesting that expression of this gene may be used to distinguish adult and fetal liver.

In addition, there is substantial expression of this gene associated with cancer cell lines. Therefore, therapeutic modulation of the activity of this gene or its protein product, through the use of small molecule drugs, protein therapeutics or antibodies, might be beneficial in the treatment of cancer.

Panel 4D Summary: Ag3288 The CG57570-01 gene is expressed at high to moderate levels in a wide range of cell types of significance in the immune response in health and disease, with the highest expression being detected in activated B cells (CT=25). Therefore, targeting the CG57570-01 gene product with a small molecule drug or antibody therapeutic may modulate the functions of cells of the immune system, and particularly B cells, and lead to improvement of the symptoms of patients suffering from rheumatoid diseases, B hyperglobulinemia and autoimmune disorders.

NOV14

Expression of gene CG57593-01 was assessed using the primer-probe set Ag3292, described in Table LA.

TABLE LA
Probe Name Ag3292

| Primers | Sequences | Length | Start Position | SEQ ID NO |
|---|---|---|---|---|
| Forward | 5′-tggtcataacatcaatcccaat-3′ | 22 | 957 | 291 |
| Probe | TET-5′-tggcttcatggtgatattcacactct-3′-TAMRA | 26 | 991 | 292 |
| Reverse | 5′-gaaagccaacaccaaagaaag-3′ | 21 | 1031 | 293 |

CNS_neurodegeneration_v1.0 Summary: Ag3292 Expression of the CG57593-01 gene is low/undetectable (CTs>35) across all of the samples on this panel (data not shown).

General_screening_panel_v1.4 Summary: Ag3292 Expression of the CG57593-01 gene is low/undetectable (CTs>35) across all of the samples on this panel (data not shown).

Panel 4D Summary: Ag3292 Expression of the CG57593-01 gene is low/undetectable (CTs>35) across all of the samples on this panel (data not shown).

NOV15

Expression of NOV15/CG57652-01 was assessed using the primer-probe sets Ag1762 and Ag1763, described in Tables MA and MB. Results of the RTQ-PCR runs are shown in Tables MC and MD.

Table MA. Probe Name Ag1762

TABLE MA
Probe Name Ag1762

| Primers | Sequences | Length | Start Position | SEQ ID NO |
|---|---|---|---|---|
| Forward | 5′-caaacagggactgagctgtaac-3′ | 22 | 766 | 295 |
| Probe | TET-5′-tacactgttcacgaccagtgtgccat-3′-TAMRA | 26 | 797 | 296 |
| Reverse | 5′-gcataggtgctgacttcacaa-3′ | 21 | 835 | 297 |

TABLE MB
Probe Name Ag1763

| Primers | Sequences | Length | Start Position | SEQ ID NO |
|---|---|---|---|---|
| Forward | 5′-ggtgctctggaagttccagtat-3′ | 22 | 1255 | 298 |
| Probe | TET-5′-accctcgacaggtgttcaacctccta-3′-TAMRA | 26 | 1284 | 299 |
| Reverse | 5′-cttgaataatcggagccctatc-3′ | 22 | 1324 | 300 |

TABLE MC
Panel 1.3D

| | Rel. Exp. (%) | Rel. Exp. (%) | | Rel. Exp. (%) | Rel. Exp. (%) |
|---|---|---|---|---|---|
| | Ag1762, Run | Ag1763, Run | | Ag1762, Run | Ag1763, Run |
| Tissue Name | 157720756 | 157717718 | Tissue Name | 157720756 | 157717718 |
| Liver adenocarcinoma | 26.6 | 25.7 | Kidney (fetal) | 2.5 | 3.7 |
| Pancreas | 1.6 | 2.8 | Renal ca. 786-0 | 0.2 | 0.1 |
| Pancreatic ca. | 9.2 | 8.1 | Renal ca. A498 | 20.3 | 18.7 |
| CAPAN 2 | | | | | |
| Adrenal gland | 6.7 | 6.2 | Renal ca. RXF 393 | 0.7 | 0.6 |
| Thyroid | 5.0 | 3.4 | Renal ca. ACHN | 4.7 | 4.4 |
| Salivary gland | 13.6 | 23.7 | Renal ca. UO-31 | 21.9 | 15.8 |
| Pituitary gland | 3.9 | 3.9 | Renal ca. TK-10 | 1.7 | 2.2 |
| Brain (fetal) | 1.7 | 3.6 | Liver | 1.1 | 1.0 |
| Brain (whole) | 6.6 | 8.2 | Liver (fetal) | 3.7 | 3.5 |
| Brain (amygdala) | 6.5 | 8.2 | Liver ca. | 2.4 | 1.3 |
| | | | (hepatoblast) | | |
| | | | HepG2 | | |
| Brain (cerebellum) | 2.1 | 3.4 | Lung | 15.9 | 16.7 |
| Brain (hippocampus) | 28.9 | 35.6 | Lung (fetal) | 7.7 | 12.6 |
| Brain (substantia | 0.8 | 1.3 | Lung ca. (small | 3.5 | 1.6 |
| nigra) | | | cell) LX-1 | | |
| Brain (thalamus) | 1.9 | 2.4 | Lung ca. (small | 2.4 | 2.3 |
| | | | cell) NCI-H69 | | |
| Cerebral Cortex | 25.5 | 18.3 | Lung ca. (s. cell | 3.9 | 4.1 |
| | | | var.) SHP-77 | | |
| Spinal cord | 2.2 | 3.3 | Lung ca. (large | 0.9 | 1.1 |
| | | | cell)NCI-H460 | | |
| glio/astro U87-MG | 4.9 | 5.6 | Lung ca. (non-sm. | 5.0 | 3.5 |
| | | | cell) A549 | | |
| glio/astro U-118-MG | 46.7 | 38.2 | Lung ca. (non- | 1.3 | 2.3 |
| | | | s. cell) NCI-H23 | | |
| astrocytoma SW1783 | 7.3 | 3.8 | Lung ca. (non- | 1.5 | 2.4 |
| | | | s. cell) HOP-62 | | |
| neuro*; met SK-N-AS | 24.1 | 25.7 | Lung ca. (non-s. cl) | 3.0 | 2.9 |
| | | | NCI-H522 | | |
| astrocytoma SF-539 | 4.8 | 5.8 | Lung ca. (squam.) | 7.4 | 7.9 |
| | | | SW 900 | | |
| astrocytoma SNB-75 | 15.1 | 10.0 | Lung ca. (squam.) | 0.2 | 0.3 |
| | | | NCI-H596 | | |
| glioma SNB-19 | 1.5 | 2.1 | Mammary gland | 7.9 | 9.2 |
| glioma U251 | 3.0 | 1.6 | Breast ca.* (pl. ef) | 16.5 | 1.2 |
| | | | MCF-7 | | |
| glioma SF-295 | 5.1 | 5.8 | Breast ca.* (pl. ef) | 8.6 | 8.1 |
| | | | MDA-MB-231 | | |
| Heart (fetal) | 3.2 | 1.4 | Breast ca.* (pl. ef) | 5.4 | 5.6 |
| | | | T47D | | |
| Heart | 1.3 | 0.9 | Breast ca. BT-549 | 6.5 | 5.9 |
| Skeletal muscle (fetal) | 6.7 | 5.8 | Breast ca. MDA-N | 2.4 | 1.2 |
| Skeletal muscle | 0.8 | 0.3 | Ovary | 13.2 | 10.8 |
| Bone marrow | 15.0 | 14.7 | Ovarian ca. | 1.6 | 1.3 |
| | | | OVCAR-3 | | |
| Thymus | 100.0 | 100.0 | Ovarian ca. | 2.0 | 0.9 |
| | | | OVCAR-4 | | |
| Spleen | 24.5 | 39.0 | Ovarian ca. | 3.9 | 3.9 |
| | | | OVCAR-5 | | |
| Lymph node | 26.6 | 40.1 | Ovarian ca. | 11.8 | 9.2 |
| | | | OVCAR-8 | | |
| Colorectal | 7.5 | 8.7 | Ovarian ca. | 1.5 | 2.7 |
| | | | IGROV-1 | | |
| Stomach | 7.5 | 13.8 | Ovarian ca.* | 8.9 | 6.0 |
| | | | (ascites) SK-OV-3 | | |
| Small intestine | 19.8 | 19.5 | Uterus | 5.5 | 8.2 |
| Colon ca. SW480 | 5.1 | 6.1 | Placenta | 14.2 | 12.9 |
| Colon ca.* | 0.9 | 1.2 | Prostate | 7.6 | 10.7 |
| SW620(SW480 met) | | | | | |
| Colon ca. HT29 | 1.3 | 0.7 | Prostate ca.* (bone | 10.7 | 11.1 |
| | | | met)PC-3 | | |
| Colon ca. HCT-116 | 5.4 | 3.2 | Testis | 9.0 | 10.7 |
| Colon ca. CaCo-2 | 3.8 | 3.2 | Melanoma | 5.9 | 4.4 |
| | | | Hs688(A).T | | |
| Colon ca. | 26.1 | 19.5 | Melanoma* (met) | 4.7 | 2.6 |
| tissue(ODO3866) | | | Hs688(B).T | | |
| Colon ca. HCC-2998 | 8.1 | 4.3 | Melanoma UACC- | 0.3 | 0.4 |
| | | | 62 | | |
| Gastric ca.* (liver | 21.6 | 21.0 | Melanoma M14 | 0.1 | 0.0 |
| met) NCI-N87 | | | | | |
| Bladder | 3.7 | 2.0 | Melanoma LOX | 2.2 | 0.7 |
| | | | IMVI | | |
| Trachea | 46.7 | 50.3 | Melanoma* (met) | 0.4 | 0.3 |
| Kidney | 0.2 | 0.7 | Adipose | 2.6 | 2.5 |

TABLE MD
Panel 5 Islet

| | Rel. Exp. (%) | | Rel. Exp. (%) |
|---|---|---|---|
| | Ag1763, Run | | Ag1763, Run |
| Tissue Name | 242285275 | Tissue Name | 242285275 |
| 97457_Patient-02go_adipose | 22.2 | 94709_Donor 2 AM - A_adipose | 55.9 |
| 97476_Patient-07sk_skeletal | 7.5 | 94710_Donor 2 AM - B_adipose | 11.0 |
| muscle | | | |
| 97477_Patient-07ut_uterus | 13.8 | 94711_Donor 2 AM - C_adipose | 16.0 |
| 97478_Patient-07pl_placenta | 34.2 | 94712_Donor 2 AD - A_adipose | 68.8 |
| 99167_Bayer Patient 1 | 47.3 | 94713_Donor 2 AD - B_adipose | 56.6 |
| 97482_Patient-08ut_uterus | 12.0 | 94714_Donor 2 AD - C_adipose | 59.5 |
| 97483_Patient-08pl_placenta | 29.5 | 94742_Donor 3 U - A_Mesenchymal | 33.2 |
| | | Stem Cells | |
| 97486_Patient-09sk_skeletal | 1.9 | 94743_Donor 3 U - B_Mesenchymal | 26.4 |
| muscle | | Stem Cells | |
| 97487_Patient-09ut_uterus | 21.5 | 94730_Donor 3 AM - A_adipose | 73.7 |
| 97488_Patient-09pl_placenta | 23.8 | 94731_Donor 3 AM - B_adipose | 26.1 |
| 97492_Patient-10ut_uterus | 23.7 | 94732_Donor 3 AM - C_adipose | 26.4 |
| 97493_Patient-10pl_placenta | 65.1 | 94733_Donor 3 AD - A_adipose | 55.5 |
| 97495_Patient-11go_adipose | 23.3 | 94734_Donor 3 AD - B_adipose | 31.0 |
| 97496_Patient-11sk_skeletal | 4.4 | 94735_Donor 3 AD - C_adipose | 41.5 |
| muscle | | | |
| 97497_Patient-11ut_uterus | 12.9 | 77138_Liver_HepG2untreated | 35.6 |
| 97498_Patient-11pl_placenta | 15.1 | 73556_Heart_Cardiac stromal cells | 44.8 |
| | | (primary) | |
| 97500_Patient-12go_adipose | 18.2 | 81735_Small Intestine | 74.7 |
| 97501_Patient-12sk_skeletal | 9.2 | 72409_Kidney_Proximal Convoluted | 6.4 |
| muscle | | Tubule | |
| 97502_Patient-12ut_uterus | 18.9 | 82685_Small intestine_Duodenum | 22.7 |
| 97503_Patient-12pl_placenta | 18.7 | 90650_Adrenal_Adrenocortical | 11.3 |
| | | adenoma | |
| 94721_Donor 2 U - | 28.3 | 72410_Kidney_HRCE | 100.0 |
| A_Mesenchymal Stem Cells | | | |
| 94722_Donor 2 U - | 22.8 | 72411_Kidney_HRE | 62.0 |
| B_Mesenchymal Stem Cells | | | |
| 94723_Donor 2 U - | 37.1 | 73139_Uterus_Uterine smooth muscle | 16.4 |
| C_Mesenchymal Stem Cells | | cells | |

Panel 1.3D Summary: Ag1762/Ag1763 Results from experiments using two different probe/primer sets are in excellent agreement. In both experiments, the CG57652-01 gene is expressed at highest levels in thymus tissue (CT=27.3/25.8).

In addition, this gene is expressed at moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

This gene is expressed in a number of tissues with metabolic or endocrine function, including adipose, adrenal gland, gastrointestinal tract, pancreas, skeletal muscle and thyroid. Therefore, therapeutic modulation of the activity of this gene may prove useful in the treatment of endocrine/metabolically related diseases, such as obesity and diabetes. The CG57652-01 gene encodes a variant of the diacylglycerol kinase-alpha gene (ref. 1). It has been shown that unsaturated fatty acids can lead to increased protein kinase C (PKC) activation by a deactivation of diacylglycerol kinase-alpha (ref. 2-3). Therefore, therapeutic modulation of the activity of the CG57652-01 gene or its protein product using small molecule drugs may be used to combat the negative effects of increased fatty acids in patients with diabetes.

References:

1. Schaap D, de Widt J, van der Wal J, Vandekerckhove J, van Damme J, Gussow D, Ploegh H L, van Blitterswijk W J, van der Bend R L. Purification, cDNA-cloning and expression of human diacylglycerol kinase. FEBS Lett Nov. 26, 1990;275(1-2):151-8

Diacylglycerol (DG) kinase attenuates the level of the second messenger DG in signal transduction, and therefore possibly modulates protein kinase C (PKC). DG kinase was purified to homogeneity from human white blood cells, showing an Mr of 86 kDa as determined by SDS-PAGE and gel filtration. Two amino acid sequences of tryptic peptides from DG kinase were determined and degenerate oligonucleotides were prepared and used in the polymerase chain reaction. An amplified DNA fragment was subsequently used to clone the full-length human DG kinase cDNA. This sequence is the human homolog of a porcine DG kinase cDNA sequence reported recently. The sequence contains a double EF-hand structure typical for Ca2+ binding proteins. DG kinase further contains a double cysteine repeat that is present in all PKC isoforms, where it constitutes the phorbol ester (and most likely diacylglycerol) binding site. Therefore we speculate that the double cysteine repeat in DG kinase is involved in DG binding. DG kinase is transcribed as a single mRNA of 3.2 kb, that is highly expressed in T-lymphocytes. The human DG kinase cDNA when transfected in mammalian cells (COS-7) results in a 6-7-fold increase of DG kinase activity.

PMID: 2175712

2. Du X, Jiang Y, Qian W, Lu X, Walsh J P. Fatty acids inhibit growth-factor-induced diacylglycerol kinase alpha activation in vascular smooth-muscle cells. Biochem J Jul. 1, 2001;357(Pt 1):275-82.

We have previously shown that unsaturated fatty acids amplify platelet-derived-growth-factor (PDGF)-induced protein kinase C (PKC) activation in vascular smooth-muscle cells (VSMCs). Diacylglycerol-induced PKC activation is normally terminated by diacylglycerol kinases (DGKs). We thus hypothesized that fatty acids act by inhibiting a DGK. Fractionation of VSMC extracts demonstrated that the DGK alpha isoform was the major DGK activity present. PDGF markedly increased the DGK activity of cultured cells. An inhibitor selective for the DGK alpha isoform, R59949 [3-[2-[4-(bis-(4-fluorophenyl)methlylene] piperidin-1-yl)ethyl-2,3-dihydro-2-thioxo-4(1F)-quinazolinone], abolished the growth-factor-induced increase in DGK activity, but had little effect on basal activity. PDGF thus selectively activates DGKalpha. Epidermal growth factor and alpha-thrombin stimulated total DGK activity similarly to PDGF. Activation by epidermal growth factor was sensitive to R59949, again suggesting involvement of DGKalpha. However, the alpha-thrombin-induced activity was unaffected by this agent. Unsaturated fatty acids inhibited growth-factor-induced DGKalpha activation, but had no effect on basal activity. Fatty acids also amplified the PDGF-induced increase in cell diacylglycerol content. These results indicate that inhibition of DGKalpha contributes to fatty-acid-induced amplification of PKC activation. Increased levels of fatty acids in diabetes may thus contribute to chronic PKC activation associated with this disorder.

PMID: 11415460

3. Koya D, Lee I K, Ishii H, Kanoh H, King G L. Prevention of glomerular dysfunction in diabetic rats by treatment with d-alpha-tocopherol. J Am Soc Nephrol 1997 March;8(3):426-35.

Because d-alpha-tocopherol (vitamin E) has been shown to decrease diacylglycerol (DAG) levels and prevent the activation of protein kinase C (PKC), which is associated with retinal and renal dysfunctions in diabetes, the study presented here characterized the effect of d-alpha-tocopherol treatment to prevent glomerular hyperfiltration and increased albumiinuria as well as PKC activities in streptozotocin (STZ)-induced diabetic rats. Two weeks after the induction of diabetes, total DAG content and PKC activity in glomeruli were significantly increased in diabetic rats by 106.4+/−16.8% and 66.4+/−8.4%, respectively, compared with control rats. Intraperitoneal injection of d-alpha-tocopherol (40 mg/kg of body weight) every other day prevented the increases in total DAG content and PKC activity in glomeruli of diabetic rats. Glomerular filtration rate (GFR) and filtration fraction (FF) were significantly elevated to 4.98+/−0.34 mL/min and 0.36+/−0.05, respectively, in diabetic rats, compared with 2.90+/−0.14 mL/min and 0.25+/−0.02, respectively, in control rats. These hemodynamic abnormalities in diabetic rats were normalized to 2.98+/−0.09 mL/min and 0.24+/−0.01, respectively, by d-alpha-tocopherol. Albuminuria in 10-wk diabetic rats was significantly increased to 9.1+/−2.2 mg/day compared with 1.2+/−0.3 mg/day in control rats, whereas d-alpha-tocopherol treatment improved albumin excretion rate to 2.4+/−0.6 mg/day in diabetic rats. To clarify the mechanism of d-alpha-tocopherol's effect on DAG-PKC pathway, the activity and protein levels of DAG kinase alpha and gamma, which metabolize DAG to phosphatidic acid, were examined. Treatment with d-alpha-tocopherol increased DAG kinase activity in the glomeruli of both control and diabetic rats, by 22.6+/−3.6% and 28.5+/−2.3% respectively, although no differences were observed in the basal DAG kinase activity between control and diabetic rats. Because immunoblotting studies did not exhibit any difference in the protein levels of DAG kinase alpha and gamma, the effect of d-alpha-tocopherol is probably modulating the enzyme kinetics of DAG kinase. These findings suggest that the increases in DAG-PKC pathway play an important role for the development of glomerular hyperfiltration and increased albuminuria in diabetes and that d-alpha-tocopherol treatment could be preventing early changes of diabetic renal dysfunctions by normalizing the increases in DAG and PKC levels in glomerular cells.

PMID: 9071711

Panel 5 Islet Summary: Ag1763 The CG57652-01 gene is expressed at low levels in the majority of samples on this panel. This gene has a low level of expression in the islets of Langerhans (patient 1). Diacylglycerol kinase attenuates protein kinase C activation, which may in turn, reduce insulin secretion (ref. 1). Since the secretion of insulin is enhanced by activation of protein kinase C, an inhibitor of diacylglycerol kinase encoded by the CG57652-01 gene may be a treatment for the insulin secretory defect in Type 2 diabetes.

References:

1. Formisano P, Beguinot F. The role of protein kinase C isoforms in insulin action. J Endocrinol Invest. 2001 June;24(6):460-7.

Insulin action on target tissues is mediated by specific tyrosine kinase receptors. Upon ligand binding insulin receptors autophosphorylate and phosphorylate intracellular substrates on tyrosine residues. These early events of insulin action are followed by the activation of a number of enzymes, including protein kinase C (PKC). At least 14 PKC isoforms have been identified and cloned to date. PKCs appear to play dual roles in insulin signaling. For instance, they are involved in transduction of specific insulin signals but also contribute to the generation of insulin resistance. In this article, we will analyze the experimental evidence addressing the mechanism by which insulin might activate individual PKC isoforms as well as the role of single PKCs in insulin-induced bioeffects.

PMID: 11434672

NOV16

Expression of NOV11

6/CG57562-01 was assessed using the primer-probe sets Ag3287 and Ag1179, described in Tables NA and NB. Results of the RTQ-PCR runs are shown in Tables NC, ND), NE and NF.

TABLE NA
Probe Name Ag3287

| Primers | Sequences | Length | Start Position | SEQ ID NO |
|---|---|---|---|---|
| Forward | 5′-cgctacagatgttcaagatcct-3′ | 22 | 3018 | 301 |
| Probe | TET-5′-ctacagccagagcgtcctctacctgg-3′-TAMRA | 26 | 3064 | 302 |
| Reverse | 5′-cctggaagtcactgaacttgac-3′ | 22 | 3095 | 303 |

TABLE NB
Probe Name Ag1179

| Primers | Sequences | Length | Start Position | SEQ ID NO |
|---|---|---|---|---|
| Forward | 5′-cgctacagatgttcaagatcct-3′ | 22 | 3018 | 304 |
| Probe | TET-5′-ctacagccagagcgtcctctacctgg-3′-TAMRA | 26 | 3064 | 305 |
| Reverse | 5′-cctggaagtcactgaacttgac-3′ | 22 | 3095 | 306 |

TABLE NC
CNS_neurodegeneration_v1.0

| | Rel. Exp. (%) | | Rel. Exp. (%) |
|---|---|---|---|
| | Ag3287, Run | | Ag3287, Run |
| Tissue Name | 210058659 | Tissue Name | 210058659 |
| AD 1 Hippo | 23.2 | Control (Path) 3 Temporal | 6.8 |
| | | Ctx | |
| AD 2 Hippo | 35.6 | Control (Path) 4 Temporal | 42.0 |
| | | Ctx | |
| AD 3 Hippo | 11.8 | AD 1 Occipital Ctx | 19.9 |
| AD 4 Hippo | 11.9 | AD 2 Occipital Ctx | 1.6 |
| | | (Missing) | |
| AD 5 Hippo | 100.0 | AD 3 Occipital Ctx | 10.2 |
| AD 6 Hippo | 72.2 | AD 4 Occipital Ctx | 20.4 |
| Control 2 Hippo | 33.7 | AD 5 Occipital Ctx | 42.6 |
| Control 4 Hippo | 15.9 | AD 6 Occipital Ctx | 14.9 |
| Control (Path) 3 Hippo | 9.1 | Control 1 Occipital Ctx | 5.0 |
| AD 1 Temporal Ctx | 34.2 | Control 2 Occipital Ctx | 60.3 |
| AD 2 Temporal Ctx | 37.9 | Control 3 Occipital Ctx | 22.4 |
| AD 3 Temporal Ctx | 10.2 | Control 4 Occipital Ctx | 6.7 |
| AD 4 Temporal Ctx | 26.4 | Control (Path) 1 Occipital | 86.5 |
| | | Ctx | |
| AD 5 Inf Temporal Ctx | 88.9 | Control (Path) 2 Occipital | 12.5 |
| | | Ctx | |
| AD 5 Sup Temporal Ctx | 64.6 | Control (Path) 3 Occipital | 3.5 |
| | | Ctx | |
| AD 6 Inf Temporal Ctx | 53.2 | Control (Path) 4 Occipital | 26.2 |
| | | Ctx | |
| AD 6 Sup Temporal Ctx | 62.0 | Control 1 Parietal Ctx | 10.4 |
| Control 1 Temporal Ctx | 8.2 | Control 2 Parietal Ctx | 52.5 |
| Control 2 Temporal Ctx | 39.8 | Control 3 Parietal Ctx | 21.9 |
| Control 3 Temporal Ctx | 17.6 | Control (Path) 1 Parietal | 62.4 |
| | | Ctx | |
| Control 3 Temporal Ctx | 6.3 | Control (Path) 2 Parietal | 24.1 |
| | | Ctx | |
| Control (Path) 1 Temporal | 54.0 | Control (Path) 3 Parietal | 6.5 |
| Ctx | | Ctx | |
| Control (Path) 2 Temporal | 41.2 | Control (Path) 4 Parietal | 32.5 |
| Ctx | | Ctx | |

TABLE ND
General_screening_panel_v1.4

| | Rel. Exp. (%) | | Rel. Exp. (%) |
|---|---|---|---|
| | Ag3287, Run | | Ag3287, Run |
| Tissue Name | 216516908 | Tissue Name | 216516908 |
| Adipose | 4.8 | Renal ca. TK-10 | 26.4 |
| Melanoma* Hs688(A).T | 23.0 | Bladder | 19.9 |
| Melanoma* Hs688(B).T | 27.9 | Gastric ca. (liver met.) NCI-N87 | 44.4 |
| Melanoma* M14 | 31.6 | Gastric ca. KATO III | 39.8 |
| Melanoma* LOXIMVI | 33.7 | Colon ca. SW-948 | 24.3 |
| Melanoma* SK-MEL-5 | 31.0 | Colon ca. SW480 | 39.8 |
| Squamous cell carcinoma | 18.4 | Colon ca.* (SW480 met) | 33.0 |
| SCC-4 | | SW620 | |
| Testis Pool | 12.2 | Colon ca. HT29 | 10.0 |
| Prostate ca.* (bone met) | 49.7 | Colon ca. HCT-116 | 48.3 |
| PC-3 | | | |
| Prostate Pool | 6.8 | Colon ca. CaCo-2 | 46.7 |
| Placenta | 23.0 | Colon cancer tissue | 19.1 |
| Uterus Pool | 4.5 | Colon ca. SW1116 | 11.7 |
| Ovarian ca. OVCAR-3 | 22.7 | Colon ca. Colo-205 | 15.9 |
| Ovarian ca. SK-OV-3 | 48.0 | Colon ca. SW-48 | 10.9 |
| Ovarian ca. OVCAR-4 | 20.6 | Colon Pool | 16.5 |
| Ovarian ca. OVCAR-5 | 38.4 | Small Intestine Pool | 14.5 |
| Ovarian ca. IGROV-1 | 34.2 | Stomach Pool | 9.2 |
| Ovarian ca. OVCAR-8 | 13.5 | Bone Marrow Pool | 6.8 |
| Ovary | 11.7 | Fetal Heart | 18.0 |
| Breast ca. MCF-7 | 33.0 | Heart Pool | 5.2 |
| Breast ca. MDA-MB-231 | 49.3 | Lymph Node Pool | 19.8 |
| Breast ca. BT 549 | 100.0 | Fetal Skeletal Muscle | 10.5 |
| Breast ca. T47D | 76.8 | Skeletal Muscle Pool | 5.8 |
| Breast ca. MDA-N | 24.0 | Spleen Pool | 12.0 |
| Breast Pool | 18.4 | Thymus Pool | 19.1 |
| Trachea | 16.6 | CNS cancer (glio/astro) U87- | 36.1 |
| | | MG | |
| Lung | 5.5 | CNS cancer (glio/astro) U- | 65.5 |
| | | 118-MG | |
| Fetal Lung | 42.6 | CNS cancer (neuro; met) SK- | 69.3 |
| | | N-AS | |
| Lung ca. NCI-N417 | 18.3 | CNS cancer (astro) SF-539 | 22.4 |
| Lung ca. LX-1 | 26.1 | CNS cancer (astro) SNB-75 | 49.0 |
| Lung ca. NCI-H146 | 17.8 | CNS cancer (glio) SNB-19 | 31.9 |
| Lung ca. SHP-77 | 37.9 | CNS cancer (glio) SF-295 | 84.7 |
| Lung ca A549 | 24.1 | Brain (Amygdala) Pool | 7.5 |
| Lung ca. NCI-H526 | 7.3 | Brain (cerebellum) | 42.3 |
| Lung ca. NCI-H23 | 41.8 | Brain (fetal) | 26.8 |
| Lung ca. NCI-H460 | 34.6 | Brain (Hippocampus) Pool | 8.2 |
| Lung ca. HOP-62 | 13.4 | Cerebral Cortex Pool | 9.1 |
| Lung ca. NCI-H522 | 17.6 | Brain (Substantia nigra) Pool | 9.1 |
| Liver | 5.3 | Brain (Thalamus) Pool | 13.1 |
| Fetal Liver | 21.6 | Brain (whole) | 18.4 |
| Liver ca. HepG2 | 23.0 | Spinal Cord Pool | 6.6 |
| Kidney Pool | 22.1 | Adrenal Gland | 24.0 |
| Fetal Kidney | 24.1 | Pituitary gland Pool | 6.0 |
| Renal ca. 786-0 | 20.9 | Salivary Gland | 13.3 |
| Renal ca. A498 | 23.2 | Thyroid (female) | 9.5 |
| Renal ca. ACHN | 12.4 | Pancreatic ca. CAPAN2 | 44.8 |
| Renal ca. UO-31 | 27.7 | Pancreas Pool | 21.6 |

TABLE NE
Panel 1.2

| | Rel. Exp. (%) | | Rel. Exp. (%) |
|---|---|---|---|
| | Ag1179, Run | | Ag1179, Run |
| Tissue Name | 129140472 | Tissue Name | 129140472 |
| Endothelial cells | 6.0 | Renal ca. 786-0 | 1.8 |
| Heart (Fetal) | 25.2 | Renal ca. A498 | 16.6 |
| Pancreas | 3.5 | Renal ca. RXF 393 | 0.6 |
| Pancreatic ca. CAPAN2 | 23.2 | Renal ca. ACHN | 8.0 |
| Adrenal Gland | 53.2 | Renal ca. UO-31 | 5.0 |
| Thyroid | 6.2 | Renal ca. TK-10 | 7.5 |
| Salivary gland | 26.4 | Liver | 15.6 |
| Pituitary gland | 14.4 | Liver (fetal) | 13.6 |
| Brain (fetal) | 17.9 | Liver ca. (hepatoblast) | 9.9 |
| | | HepG2 | |
| Brain (whole) | 18.7 | Lung | 10.0 |
| Brain (amygdala) | 17.7 | Lung (fetal) | 13.6 |
| Brain (cerebellum) | 15.4 | Lung ca. (small cell) LX-1 | 3.0 |
| Brain (hippocampus) | 31.0 | Lung ca. (small cell) NCI- | 3.5 |
| | | H69 | |
| Brain (thalamus) | 9.3 | Lung ca. (s. cell var.) SHP- | 4.6 |
| | | 77 | |
| Cerebral Cortex | 47.0 | Lung ca. (large cell) NCI- | 100.0 |
| | | H460 | |
| Spinal cord | 14.7 | Lungca. (non-sm. cell) | 5.0 |
| | | A549 | |
| glio/astro U87-MG | 8.7 | Lung ca. (non-s. cell) NCI- | 8.1 |
| | | H23 | |
| glio/astro U-118-MG | 13.3 | Lung ca. (non-s. cell) HOP- | 7.5 |
| | | 62 | |
| astrocytoma SW1783 | 4.7 | Lung ca. (non-s. cl) NCI- | 19.2 |
| | | H522 | |
| neuro*; met SK-N-AS | 17.9 | Lung ca. (squam.) SW 900 | 3.7 |
| astrocytoma SF-539 | 1.7 | Lung ca. (squam.) NCI- | 8.0 |
| | | H596 | |
| astrocytoma SNB-75 | 2.2 | Mammary gland | 28.9 |
| glioma SNB-19 | 9.0 | Breast ca.* (pl. ef) MCF-7 | 8.2 |
| glioma U251 | 3.1 | Breast ca.* (pl. ef) MDA- | 3.5 |
| | | MB-231 | |
| glioma SF-295 | 49.7 | Breast ca.* (pl. ef) T47D | 4.9 |
| Heart | 20.6 | Breast ca. BT-549 | 11.7 |
| Skeletal Muscle | 6.2 | Breast ca. MDA-N | 4.8 |
| Bone marrow | 7.5 | Ovary | 34.9 |
| Thymus | 12.8 | Ovarian ca. OVCAR-3 | 8.4 |
| Spleen | 9.3 | Ovarian ca. OVCAR-4 | 12.0 |
| Lymph node | 42.0 | Ovarian ca. OVCAR-5 | 9.7 |
| Colorectal Tissue | 10.8 | Ovarian ca. OVCAR-8 | 1.6 |
| Stomach | 67.8 | Ovarian ca. IGROV-1 | 8.6 |
| Small intestine | 20.3 | Ovarian ca. (ascites) SK- | 16.7 |
| | | OV-3 | |
| Colon ca. SW480 | 1.0 | Uterus | 9.2 |
| Colon ca.* SW620 (SW480 | 3.6 | Placenta | 31.2 |
| met) | | | |
| Colon ca. HT29 | 1.3 | Prostate | 29.7 |
| Colon ca.HCT-116 | 3.4 | Prostate ca.* (bone met) | 72.7 |
| | | PC-3 | |
| Colon ca. CaCo-2 | 7.2 | Testis | 45.4 |
| Colon ca. Tissue | 6.6 | Melanoma Hs688(A).T | 4.9 |
| (ODO3866) | | | |
| Colon ca. MCC-2998 | 3.6 | Melanoma* (met) | 5.1 |
| | | Hs688(B).T | |
| Gastric ca.* (liver met) | 9.6 | Melanoma UACC-62 | 34.9 |
| NCI-N87 | | | |
| Bladder | 28.3 | Melanoma M14 | 21.9 |
| Trachea | 16.8 | Melanoma LOX IMVI | 19.3 |
| Kidney | 4.6 | Melanoma* (met) SK- | 25.5 |
| | | MEL-5 | |
| Kidney (fetal) | 25.3 | | |

TABLE NF
Panel 4D

| | Rel. Exp. (%) | Rel. Exp. (%) | | Rel. Exp. (%) | Rel. Exp. (%) |
|---|---|---|---|---|---|
| | Ag1179, Run | Ag3287, Run | | Ag1179, Run | Ag3287, Run |
| Tissue Name | 139820117 | 164633941 | Tissue Name | 139820117 | 164633941 |
| Secondary Th1 act | 46.0 | 55.5 | HUVEC IL-1beta | 5.9 | 9.9 |
| Secondary Th2 act | 76.3 | 71.7 | HUVEC IFN gamma | 21.3 | 65.1 |
| Secondary Tr1 act | 38.4 | 49.7 | HUVEC TNF alpha + | 10.7 | 25.9 |
| | | | IFN gamma | | |
| Secondary Th1 rest | 15.0 | 18.2 | HUVEC TNF alpha + | 17.1 | 23.5 |
| | | | IL4 | | |
| Secondary Th2 rest | 18.8 | 18.8 | HUVEC IL-11 | 9.3 | 14.4 |
| Secondary Tr1 rest | 10.7 | 23.5 | Lung Microvascular EC | 17.6 | 26.8 |
| | | | none | | |
| Primary Th1 act | 59.5 | 52.1 | Lung Microvascular EC | 10.9 | 21.0 |
| | | | TNF alpha + IL-1beta | | |
| Primary Th2 act | 47.0 | 40.1 | Microvascular Dermal | 37.9 | 26.1 |
| | | | EC none | | |
| Primary Tr1 act | 75.3 | 68.8 | Microsvasular Dermal | 21.3 | 17.7 |
| | | | EC TNF alpha + IL- | | |
| | | | 1beta | | |
| Primary Th1 rest | 52.1 | 60.3 | Bronchial epithelium | 32.5 | 33.4 |
| | | | TNF alpha + IL1beta | | |
| Primary Th2 rest | 30.1 | 33.9 | Small airway epithelium | 11.9 | 19.8 |
| | | | none | | |
| Primary Tr1 rest | 25.3 | 25.7 | Small airway epithelium | 58.6 | 73.2 |
| | | | TNF alpha + IL-1beta | | |
| CD45RA CD4 | 25.0 | 40.1 | Coronery artery SMC | 15.6 | 22.5 |
| lymphocyte act | | | rest | | |
| CD45RO CD4 | 56.3 | 63.7 | Coronery artery SMC | 14.9 | 13.6 |
| lymphocyte act | | | TNF alpha + IL-1beta | | |
| CD8 lymphocyte act | 35.6 | 53.6 | Astrocytes rest | 15.2 | 21.8 |
| Secondary CD8 | 44.4 | 48.3 | Astrocytes TNF alpha + | 17.3 | 19.6 |
| lymphocyte rest | | | IL-1beta | | |
| Secondary CD8 | 29.1 | 21.3 | KU-812 (Basophil) rest | 39.0 | 59.9 |
| lymphocyte act | | | | | |
| CD4 lymphocyte none | 16.5 | 22.5 | KU-812 (Basophil) | 83.5 | 83.5 |
| | | | PMA/ionomycin | | |
| 2ry Th1/Th2/Tr1_anti- | 31.4 | 25.2 | CCD1106 | 16.4 | 24.1 |
| CD95 CH11 | | | (Keratinocytes) none | | |
| LAK cells rest | 40.6 | 43.8 | CCD1106 | 54.7 | 25.0 |
| | | | (Keratinocytes) | | |
| | | | TNF alpha + IL-1beta | | |
| LAK cells IL-2 | 37.1 | 42.6 | Liver cirrhosis | 6.0 | 6.8 |
| LAK cells IL-2 + IL-12 | 35.8 | 34.2 | Lupus kidney | 17.1 | 10.4 |
| LAK cells IL-2 + IFN | 39.8 | 42.0 | NCI-H292 none | 29.5 | 35.4 |
| gamma | | | | | |
| LAK cells IL-2 + IL- | 28.3 | 42.0 | NCI-H292 IL-4 | 41.8 | 73.2 |
| 18 | | | | | |
| LAK cells | 18.4 | 19.8 | NCI-H292 IL-9 | 35.4 | 48.6 |
| PMA/ionomycin | | | | | |
| NK Cells IL-2 rest | 18.3 | 26.2 | NCI-H292 IL-13 | 39.8 | 40.9 |
| Two Way MLR 3 day | 27.9 | 50.0 | NCI-H292 IFN gamma | 24.5 | 48.3 |
| Two Way MLR 5 day | 21.9 | 29.5 | HPAEC none | 20.0 | 29.5 |
| Two Way MLR 7 day | 19.3 | 19.6 | HPAEC TNF alpha + | 18.9 | 23.7 |
| | | | IL-1beta | | |
| PBMC rest | 10.8 | 11.5 | Lung fibroblast none | 20.0 | 22.8 |
| PBMC PWM | 100.0 | 94.6 | Lung fibroblast TNF | 9.9 | 15.5 |
| | | | alpha + IL-1beta | | |
| PBMC PHA-L | 62.0 | 49.0 | Lung fibroblast IL-4 | 26.2 | 42.0 |
| Ramos (B cell) none | 69.7 | 39.8 | Lung fibroblast IL-9 | 19.3 | 28.5 |
| Ramos (B cell) | 93.3 | 100.0 | Lung fibroblast IL-13 | 41.5 | 27.9 |
| ionomycin | | | | | |
| B lymphocytes PWM | 79.6 | 91.4 | Lung fibroblast IFN | 34.6 | 43.5 |
| | | | gamma | | |
| B lymphocytes | 40.6 | 59.0 | Dermal fibroblast | 47.3 | 48.3 |
| CD40L and IL-4 | | | CCD1070 rest | | |
| EOL-1 dbcAMP | 43.2 | 76.3 | Dermal fibroblast | 47.6 | 67.4 |
| | | | CCD1070 TNF alpha | | |
| EOL-1 dbcAMP | 28.9 | 23.8 | Dermal fibroblast | 31.4 | 34.6 |
| PMA/ionomycin | | | CCD1070 IL-1beta | | |
| Dendritic cells none | 30.8 | 36.3 | Dermal fibroblast IFN | 10.2 | 18.6 |
| | | | gamma | | |
| Dendritic cells LPS | 32.8 | 44.4 | Dermal fibroblast IL-4 | 23.7 | 24.8 |
| Dendritic cells anti- | 35.6 | 38.4 | IBD Colitis 2 | 2.6 | 2.8 |
| CD40 | | | | | |
| Monycytes rest | 28.1 | 37.9 | IBD Crohn's | 1.1 | 1.4 |
| Monocytes LPS | 37.9 | 24.3 | Colon | 17.0 | 23.7 |
| Macrophages rest | 58.2 | 67.4 | Lung | 13.9 | 21.6 |
| Macrophages LPS | 69.3 | 40.9 | Thymus | 39.0 | 31.6 |
| HUVEC none | 18.7 | 23.5 | Kidney | 40.6 | 63.3 |
| HUVEC starved | 24.8 | 34.9 | | | |

CNS_neurodegeneration_v1.0 Summary: Ag)3287 This panel confirms the expression of this gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3287 The CG57562-01 gene shows moderate to high expression in all samples on this panel, with the highest expression in breast cancer cell line BT 549 (CT=25.0). The widespread expression of this gene suggests that the gene product may be involved in cell differentiation and growth.

This gene is also widely expressed among tissues with metabolic and endocrine function, including adipose, skeletal muscle, heart, pancreas, liver, adrenal gland, thyroid, and pituitary gland. This expression profile suggests that this gene product may also be involved in metabolic function and that therapeutic modulation of the expression or function of this gene may be effective in the treatment of metabolic disorders, such as obesity and diabetes.

The expression profile of this gene also shows widespread expression of this gene in the brain. This suggests that the protein encoded by this gene may be important for normal neurological function. Therefore, modulation of the function or expression of this gene may be effective in the treatment of neurodegenerative disorders, such as Alzheimer's disease and Parkinson's disease.

Panel 1.2 Summary: Ag1179 Results using the Ag1179 primer pair in this panel are in good agreement with the results using Ag3287 on panel 1.4. There is moderate to high expression in all samples on this panel, with the highest level of expression in lung cancer cell line NCI-H460 (CT=23.6). Please see General_screening_panel_v1.4 summary.

Panel 4D Summary: Ag1179/Ag3287 Results from two experiments using identical probe/primer sets are in excellent agreement. This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

NOV17

Expression of NOV17/CG55914-01 was assessed using the primer-probe sets Ag4424 and Ag2842, described in Tables OA and OB. Results of the RTQ-PCR runs are shown in Tables OC, OD, OE, OF, OG and OH.

TABLE OA
Probe Name Ag4424

| Primers | Sequences | Length | Start Position | SEQ ID NO |
|---|---|---|---|---|
| Forward | 5′-ctgaaagggtccaaggtacagt-3′ | 22 | 341 | 307 |
| Probe | TET-5′-cttcaaagggtgcaagccccaagtct-3′-TAMRA | 26 | 374 | 308 |
| Reverse | 5′-gtactgccaggcttaacacctt-3′ | 22 | 413 | 308 |

TABLE OB
Probe Name Ag2842

| Primers | Sequences | Length | Start Position | SEQ ID NO |
|---|---|---|---|---|
| Forward | 5′-gaaagggtccaaggtacagttc-3′ | 22 | 343 | 310 |
| Probe | TET-5′-cttcaaagggtgcaagccccaagtct-3′-TAMRA | 26 | 374 | 311 |
| Reverse | 5′-caggcttaacaccttgtgaaag-3′ | 22 | 406 | 312 |

TABLE OC
General_screening_panel_v1.4

| | Rel. Exp. (%) | | Rel. Exp. (%) |
|---|---|---|---|
| | Ag4424, Run | | Ag4424, Run |
| Tissue Name | 219941948 | Tissue Name | 219941948 |
| Adipose | 0.0 | Renal ca. TK-10 | 6.4 |
| Melanoma* Hs688(A).T | 0.0 | Bladder | 0.3 |
| Melanoma* Hs688(B).T | 0.0 | Gastric ca. (liver met.) NCI- | 0.0 |
| | | N87 | |
| Melanoma* M14 | 0.0 | Gastric ca. KATO III | 0.2 |
| Melanoma* LOXIMVI | 0.0 | Colon ca. SW-948 | 0.0 |
| Melanoma* SK-MEL-5 | 1.1 | Colon ca. SW480 | 0.0 |
| Squamous cell carcinoma | 0.7 | Colon ca.* (SW480 met) | 0.0 |
| SCC-4 | | SW620 | |
| Testis Pool | 4.2 | Colon ca HT29 | 0.0 |
| Prostate ca.* (bone met) | 5.5 | Colon ca. HCT-116 | 0.0 |
| PC-3 | | | |
| Prostate Pool | 1.1 | Colon ca. CaCo-2 | 2.7 |
| Placenta | 0.0 | Colon cancer tissue | 0.0 |
| Uterus Pool | 0.0 | Colon ca. SW1116 | 0.0 |
| Ovarian ca. OVCAR-3 | 100.0 | Colon ca. Colo-205 | 0.0 |
| Ovarian ca. SK-OV-3 | 7.4 | Colon ca. SW-48 | 0.0 |
| Ovarian ca. OVCAR-4 | 0.0 | Colon Pool | 0.0 |
| Ovarian ca. OVCAR-5 | 1.9 | Small Intestine Pool | 0.0 |
| Ovarian ca. IGROV-1 | 0.0 | Stomach Pool | 0.4 |
| Ovarian ca. OVCAR-8 | 3.7 | Bone Marrow Pool | 0.0 |
| Ovary | 0.9 | Fetal Heart | 0.0 |
| Breast ca. MCF-7 | 1.7 | Heart Pool | 0.0 |
| Breast ca. MDA-MB-231 | 0.0 | Lymph Node Pool | 0.6 |
| Breast ca. BT 549 | 0.4 | Fetal Skeletal Muscle | 0.0 |
| Breast ca. T47D | 3.6 | Skeletal Muscle Pool | 2.6 |
| Breast ca. MDA-N | 0.0 | Spleen Pool | 0.0 |
| Breast Pool | 0.8 | Thymus Pool | 0.0 |
| Trachea | 1.7 | CNS cancer (glio/astro) U87- | 0.0 |
| | | MG | |
| Lung | 2.0 | CNS cancer (glio/astro) U- | 0.0 |
| | | 118-MG | |
| Fetal Lung | 0.3 | CNS cancer (neuro; met) SK- | 0.2 |
| | | N-AS | |
| Lungca. NCI-N417 | 0.0 | CNS cancer (astro) SF-539 | 0.0 |
| Lungca. LX-1 | 0.6 | CNS cancer (astro) SNB-75 | 0.0 |
| Lungca. NCI-H146 | 5.5 | CNS cancer (glio) SNB-19 | 0.0 |
| Lung ca. SHP-77 | 4.3 | CNS cancer (glio) SF-295 | 14.3 |
| Lung ca. A549 | 1.8 | Brain (Amygdala) Pool | 1.0 |
| Lung ca. NCI-H526 | 0.0 | Brain (cerebellum) | 0.0 |
| Lung ca. NCI-H23 | 1.6 | Brain (fetal) | 0.8 |
| Lungca. NCI-H460 | 0.3 | Brain (Hippocampus) Pool | 0.0 |
| Lung ca. HOP-62 | 0.0 | Cerebral Cortex Pool | 3.0 |
| Lungca. NCI-H522 | 1.1 | Brain (Substantia nigra) Pool | 1.4 |
| Liver | 0.0 | Brain (Thalamus) Pool | 0.3 |
| Fetal Liver | 2.0 | Brain (whole) | 0.1 |
| Liver ca. HepG2 | 0.0 | Spinal Cord Pool | 1.8 |
| Kidney Pool | 1.2 | Adrenal Gland | 0.0 |
| Fetal Kidney | 0.2 | Pituitary gland Pool | 0.4 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.2 | Pancreas Pool | 1.0 |

TABLE OD
Panel 1.3D

| | Rel. Exp. (%) | Rel. Exp. (%) | | Rel. Exp. (%) | Rel. Exp. (%) |
|---|---|---|---|---|---|
| | Ag2842, Run | Ag2842, Run | | Ag2842, Run | Ag2842, Run |
| Tissue Name | 161559587 | 165701935 | Tissue Name | 161559587 | 165701935 |
| Liver adenocarcinoma | 0.0 | 0.0 | Kidney (fetal) | 0.0 | 0.0 |
| Pancreas | 0.0 | 0.0 | Renal ca. 786-0 | 0.0 | 0.0 |
| Pancreatic ca. | 0.0 | 0.0 | Renal ca. A498 | 0.0 | 0.0 |
| CAPAN 2 | | | | | |
| Adrenal gland | 0.0 | 0.0 | Renal ca. RXF 393 | 0.0 | 0.0 |
| Thyroid | 0.0 | 0.0 | Renal ca. ACHN | 0.0 | 0.0 |
| Salivary gland | 0.0 | 0.0 | Renal ca. UO-31 | 0.0 | 0.0 |
| Pituitary gland | 0.0 | 0.0 | Renal ca. TK-10 | 0.0 | 24.0 |
| Brain (fetal) | 0.0 | 0.0 | Liver | 0.0 | 10.4 |
| Brain (whole) | 0.0 | 0.0 | Liver (fetal) | 0.0 | 0.0 |
| Brain (amygdala) | 7.8 | 0.0 | Liver ca. | 0.0 | 0.0 |
| | | | (hepatoblast) | | |
| | | | HepG2 | | |
| Brain (cerebellum) | 0.0 | 0.0 | Lung | 0.0 | 0.0 |
| Brain (hippocampus) | 0.0 | 0.0 | Lung (fetal) | 0.0 | 0.0 |
| Brain (substantia | 0.0 | 0.0 | Lung ca. (small | 0.0 | 0.0 |
| nigra) | | | cell) LX-1 | | |
| Brain (thalamus) | 0.0 | 9.3 | Lung ca. (small | 30.8 | 0.0 |
| | | | cell) NCI-H69 | | |
| Cerebral Cortex | 6.3 | 7.4 | Lung ca. (s. cell | 19.1 | 9.6 |
| | | | var.) SHP-77 | | |
| Spinal cord | 0.0 | 0.0 | Lung ca. (large | 0.0 | 9.6 |
| | | | cell)NCI-H460 | | |
| glio/astro U87-MG | 0.0 | 0.0 | Lung ca. (non-sm. | 0.0 | 6.9 |
| | | | cell) A549 | | |
| glio/astro U-118-MG | 0.0 | 0.0 | Lung ca. (non- | 0.0 | 18.0 |
| | | | s. cell) NCI-H23 | | |
| astrocytoma SW1783 | 0.0 | 0.0 | Lung ca. (non- | 0.0 | 0.0 |
| | | | s. cell) HOP-62 | | |
| neuro*; met SK-N-AS | 0.0 | 6.0 | Lung ca. (non-s. cl) | 0.0 | 4.8 |
| | | | NCI-H522 | | |
| astrocytoma SF-539 | 0.0 | 0.0 | Lung ca. (squam.) | 5.4 | 17.6 |
| | | | SW 900 | | |
| astrocytoma SNB-75 | 5.4 | 9.4 | Lung ca. (squam.) | 44.8 | 100.0 |
| | | | NCI-H596 | | |
| glioma SNB-19 | 0.0 | 0.0 | Mammary gland | 5.8 | 0.0 |
| glioma U251 | 5.3 | 0.0 | Breast ca.* (pl. ef) | 0.0 | 0.0 |
| | | | MCF-7 | | |
| glioma SF-295 | 0.0 | 30.1 | Breast ca.* (pl. ef) | 0.0 | 0.0 |
| | | | MDA-MB-231 | | |
| Heart (fetal) | 0.0 | 0.0 | Breast ca.* (pl. ef) | 0.0 | 0.0 |
| | | | T47D | | |
| Heart | 0.0 | 0.0 | Breast ca. BT-549 | 0.0 | 0.0 |
| Skeletal muscle (fetal) | 8.8 | 0.0 | Breast ca. MDA-N | 0.0 | 0.0 |
| Skeletal muscle | 6.0 | 14.7 | Ovary | 0.0 | 0.0 |
| Bone marrow | 0.0 | 0.0 | Ovarian ca. | 100.0 | 64.6 |
| | | | OVCAR-3 | | |
| Thymus | 0.0 | 0.0 | Ovarian ca. | 0.0 | 7.7 |
| | | | OVCAR-4 | | |
| Spleen | 0.0 | 0.0 | Ovarian ca. | 0.0 | 0.0 |
| | | | OVCAR-5 | | |
| Lymph node | 0.0 | 0.0 | Ovarian ca. | 42.3 | 33.9 |
| | | | OVCAR-8 | | |
| Colorectal | 7.8 | 8.5 | Ovarian ca. | 0.0 | 0.0 |
| | | | IGROV-1 | | |
| Stomach | 0.0 | 0.0 | Ovarian ca.* | 49.3 | 9.3 |
| | | | (ascites) SK-OV-3 | | |
| Small intestine | 0.0 | 0.0 | Uterus | 0.0 | 0.0 |
| Colon ca. SW480 | 0.0 | 0.0 | Placenta | 0.0 | 0.0 |
| Colon ca.* | 0.0 | 0.0 | Prostate | 4.7 | 0.0 |
| SW620(SW480 met) | | | | | |
| Colon ca. HT29 | 0.0 | 0.0 | Prostate ca.* (bone | 3.9 | 6.2 |
| | | | met)PC-3 | | |
| Colon ca. HCT-116 | 0.0 | 0.0 | Testis | 39.8 | 7.5 |
| Colon ca. CaCo-2 | 5.7 | 0.0 | Melanoma | 0.0 | 0.0 |
| | | | Hs688(A).T | | |
| Colon ca. | 0.0 | 0.0 | Melanoma* (met) | 0.0 | 0.0 |
| tissue(ODO3866) | | | Hs688(B).T | | |
| Colon ca. HCC-2998 | 0.0 | 0.0 | Melanoma UACC- | 0.0 | 0.0 |
| | | | 62 | | |
| Gastric ca.* (liver | 0.0 | 0.0 | Melanoma M14 | 0.0 | 0.0 |
| met) NCI-N87 | | | | | |
| Bladder | 2.4 | 0.0 | Melanoma LOX | 0.0 | 0.0 |
| | | | IMVI | | |
| Trachea | 0.0 | 0.0 | Melanoma* (met) | 2.7 | 0.0 |
| | | | SK-MEL-5 | | |
| Kidney | 6.0 | 0.0 | Adipose | 0.0 | 0.0 |

TABLE OE
Panel 2D

| | Rel. Exp. (%) | | Rel. Exp. (%) |
|---|---|---|---|
| | Ag2842, Run | | Ag2842, Run |
| Tissue Name | 161559907 | Tissue Name | 161559907 |
| Normal Colon | 1.6 | Kidney Margin 8120608 | 0.8 |
| CC Well to Mod Diff (ODO3866) | 0.9 | Kidney Cancer 8120613 | 7.8 |
| CC Margin (ODO3866) | 0.0 | Kidney Margin 8120614 | 3.3 |
| CC Gr.2 rectosigmoid (ODO3868) | 1.1 | Kidney Cancer 9010320 | 4.1 |
| CC Margin (ODO3868) | 0.0 | Kidney Margin 9010321 | 2.4 |
| CC Mod Diff (ODO3920) | 0.2 | Normal Uterus | 0.0 |
| CC Margin (ODO3920) | 0.5 | Uterus Cancer 064011 | 1.6 |
| CC Gr.2 ascend colon (ODO3921) | 0.0 | Normal Thyroid | 1.5 |
| CC Margin (ODO3921) | 0.0 | Thyroid Cancer 064010 | 0.9 |
| CC from Partial Hepatectomy | 2.8 | Thyroid Cancer A302152 | 0.0 |
| (ODO4309) Mets | | | |
| Liver Margin (ODO4309) | 9.9 | Thyroid Margin A302153 | 0.0 |
| Colon mets to lung (OD04451-01) | 0.0 | Normal Breast | 2.1 |
| Lung Margin (OD04451-02) | 0.0 | Breast Cancer (OD04566) | 0.0 |
| Normal Prostate 6546-1 | 0.8 | Breast Cancer (OD04590-01) | 24.0 |
| Prostate Cancer (OD04410) | 9.0 | Breast Cancer Mets | 16.5 |
| | | (OD04590-03) | |
| Prostate Margin (OD04410) | 0.0 | Breast Cancer Metastasis | 8.5 |
| | | (OD04655-05) | |
| Prostate Cancer (OD04720-01) | 3.8 | Breast Cancer 064006 | 2.1 |
| Prostate Margin (OD04720-02) | 3.6 | Breast Cancer 1024 | 2.2 |
| Normal Lung 061010 | 1.9 | Breast Cancer 9100266 | 1.8 |
| Lung Met to Muscle (ODO4286) | 2.4 | Breast Margin 9100265 | 0.0 |
| Muscle Margin (ODO4286) | 0.9 | Breast Cancer A209073 | 3.5 |
| Lung Malignant Cancer | 2.9 | Breast Margin A209073 | 7.1 |
| (OD03126) | | | |
| Lung Margin (OD03126) | 0.7 | Normal Liver | 4.0 |
| Lung Cancer (OD04404) | 0.0 | Liver Cancer 064003 | 2.6 |
| Lung Margin (OD04404) | 0.0 | Liver Cancer 1025 | 1.8 |
| Lung Cancer (OD04565) | 2.5 | Liver Cancer 1026 | 0.0 |
| Lung Margin (OD04565) | 0.0 | Liver Cancer 6004-T | 0.0 |
| Lung Cancer (OD04237-01) | 4.4 | Liver Tissue 6004-N | 3.0 |
| Lung Margin (OD04237-02) | 0.0 | Liver Cancer 6005-T | 0.0 |
| Ocular Mel Met to Liver | 1.2 | Liver Tissue 6005-N | 0.0 |
| (ODO4310) | | | |
| Liver Margin (OD04310) | 6.8 | Normal Bladder | 1.9 |
| Melanoma Mets to Lung | 0.0 | Bladder Cancer 1023 | 0.7 |
| (OD04321) | | | |
| Lung Margin (OD04321) | 0.0 | Bladder Cancer A302173 | 6.0 |
| Normal Kidney | 14.6 | Bladder Cancer (OD04718- | 0.0 |
| | | 01) | |
| Kidney Ca, Nuclear grade 2 | 0.0 | Bladder Normal Adjacent | 0.0 |
| (OD04338) | | (OD04718-03) | |
| Kidney Margin (OD04338) | 4.7 | Normal Ovary | 0.0 |
| Kidney Ca Nuclear grade 1/2 | 0.0 | Ovarian Cancer 064008 | 12.6 |
| (OD04339) | | | |
| Kidney Margin (OD04339) | 4.0 | Ovarian Cancer (OD04768- | 100.0 |
| | | 07) | |
| Kidney Ca, Clear cell type | 0.0 | Ovary Margin (OD04768-08) | 0.0 |
| (OD04340) | | | |
| Kidney Margin (OD04340) | 7.3 | Normal Stomach | 0.0 |
| Kidney Ca, Nuclear grade 3 | 0.0 | Gastric Cancer 9060358 | 0.0 |
| (OD04348) | | | |
| Kidney Margin (OD04348) | 1.0 | Stomach Margin 9060359 | 0.0 |
| Kidney Cancer (OD04622-01) | 0.0 | Gastric Cancer 9060395 | 0.0 |
| Kidney Margin (OD04622-03) | 0.8 | Stomach Margin 9060394 | 0.0 |
| Kidney Cancer (OD04450-01) | 0.7 | Gastric Cancer 9060397 | 0.0 |
| Kidney Margin (OD04450-03) | 3.9 | Stomach Margin 9060396 | 0.0 |
| Kidney Cancer 8120607 | 0.0 | Gastric Cancer 064005 | 0.0 |

TABLE OF
Panel 3D

| | Rel. Exp. (%) | | Rel. Exp. (%) |
|---|---|---|---|
| | Ag2842, Run | | Ag2842, Run |
| Tissue Name | 164843469 | Tissue Name | 164843469 |
| Daoy- Medulloblastoma | 0.0 | Ca Ski- Cervical epidermoid carcinoma | 0.0 |
| | | (metastasis) | |
| TE671- Medulloblastoma | 0.0 | ES-2- Ovarian clear cell carcinoma | 0.0 |
| D283 Med- Medulloblastoma | 0.0 | Ramos- Stimulated with | 0.0 |
| | | PMA/ionomycin 6 h | |
| PFSK-1- Primitive | 0.0 | Ramos- Stimulated with | 0.0 |
| Neuroectodermal | | PMA/ionomycin 14 h | |
| XF-498-CNS | 0.0 | MEG-01- Chronic myelogenous | 0.0 |
| | | leukemia (megokaryoblast) | |
| SNB-78- Glioma | 0.0 | Raji- Burkitl's lymphoma | 0.0 |
| SF-268- Glioblastoma | 0.0 | Daudi- Burkitt's lymphoma | 0.0 |
| T98G- Glioblastoma | 0.0 | U266- B-cell plasmacytoma | 33.4 |
| SK-N-SH- Neuroblastoma | 0.0 | CA46- Burkitt's lymphoma | 0.0 |
| (metastasis) | | | |
| SF-295- Glioblastoma | 0.0 | RL- non-Hodgkin's B-cell lymphoma | 0.0 |
| Cerebellum | 80.7 | JMI- pre-B-cell lymphoma | 0.0 |
| Cerebellum | 0.0 | Jurkat- T cell leukemia | 0.0 |
| NCI-H292- Mucoepidermoid | 15.4 | TF-1- Erythroleukemia | 0.0 |
| lung carcinoma | | | |
| DMS-114- Small cell lung cancer | 0.0 | HUT 78- T-cell lymphoma | 0.0 |
| DMS-79- Small cell lung cancer | 0.0 | U937- Histiocytic lymphoma | 0.0 |
| NCI-H146- Small cell lung | 89.5 | KU-812- Myclogenous leukemia | 0.0 |
| cancer | | | |
| NCI-H526- Small cell lung | 0.0 | 769-P- Clear cell renal carcinoma | 0.0 |
| cancer | | | |
| NCI-N417- Small cell lung | 0.0 | Caki-2- Clear cell renal carcinoma | 0.0 |
| cancer | | | |
| NCI-H82- Small cell lung cancer | 0.0 | SW 839- Clear cell renal carcinoma | 1.8 |
| NCI-H157- Squamous cell lung | 0.0 | G401- Wilms' tumor | 0.0 |
| cancer (metastasis) | | | |
| NCI-H1155- Large cell lung | 13.2 | Hs766T- Pancreatic carcinoma (LN | 0.0 |
| cancer | | metastasis) | |
| NCI-H1299- Large cell lung | 0.0 | CAPAN-1- Pancreatic adenocarcinoma | 0.0 |
| cancer | | (liver metastasis) | |
| NCI-H727- Lung carcinoid | 0.0 | SU86.86- Pancreatic carcinoma (liver | 0.0 |
| | | metastasis) | |
| NCI-UMC-11- Lung carcinoid | 54.0 | BxPC-3- Pancreatic adenocarcinoma | 0.0 |
| LX-1- Small cell lung cancer | 0.0 | HPAC- Pancreatic adenocarcinoma | 0.0 |
| Colo-205- Colon cancer | 0.0 | MIA PaCa-2- Pancreatic carcinoma | 0.0 |
| KM12- Colon cancer | 0.0 | CFPAC-1- Pancreatic cuctal | 59.9 |
| | | adenocarcinoma | |
| KM20L2- Colon cancer | 0.0 | PANC-1- Pancreatic epithelioid ductal | 0.0 |
| | | carcinoma | |
| NCI-H716- Colon cancer | 0.0 | T24- Bladder carcinoma (transitional | 0.0 |
| | | cell) | |
| SW-48- Colon adenocarcinoma | 0.0 | 5637- Bladder carcinoma | 7.2 |
| SW1116- Colon adenocarcinoma | 0.0 | HT-1197- Bladder carcinoma | 36.6 |
| LS 174T- Colon adenocarcinoma | 0.0 | UM-UC-3- Bladder carcinma | 0.0 |
| | | (transitional cell) | |
| SW-948- Colon adenocarcinoma | 0.0 | A204- Rhabdomyosarcoma | 0.0 |
| SW-480- Colon adenocarcinoma | 0.0 | HT-1080- Fibrosarcoma | 100.0 |
| NCI-SNU-5- Gastric carcinoma | 0.0 | MG-63- Osteosarcoma | 0.0 |
| KATO III- Gastric carcinoma | 0.0 | SK-LMS-1- Leiomyosarcoma (vulva) | 0.0 |
| NCI-SNU-16- Gastric carcinoma | 0.0 | SJRH30- Rhabdomyosarcoma (met to | 0.0 |
| | | bone marrow) | |
| NCI-SNU-1- Gastric carcinoma | 0.0 | A431- Epidermoid carcinoma | 0.0 |
| RF-1- Gastric adenocarcinoma | 0.0 | WM266-4- Melanoma | 0.0 |
| RF-48- Gastric adenocarcinoma | 0.0 | DU 145- Prostate carcinoma (brain | 0.0 |
| | | metastasis) | |
| MKN-45- Gastric carcinoma | 0.0 | MDA-MB-468- Breast | 44.8 |
| | | adenocarcinoma | |
| NCI-N87- Gastric carcinoma | 0.0 | SCC-4- Squamous cell carcinoma of | 0.0 |
| | | tongue | |
| OVCAR-5- Ovarian carcinoma | 0.0 | SCC-9- Squamous cell carcinoma of | 0.0 |
| | | tongue | |
| RL95-2- Uterine carcinoma | 0.0 | SCC-15- Squamous cell carcinoma of | 0.0 |
| | | tongue | |
| HelaS3- Cervical | 0.0 | CAL 27- Squamous cell carcinoma of | 36.3 |
| adenocarcinoma | | tongue | |

TABLE OG
Panel 4D

| | Rel. Exp. (%) | | Rel. Exp. (%) |
|---|---|---|---|
| | Ag2842, Run | | Ag2842, Run |
| Tissue Name | 159841917 | Tissue Name | 159841917 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVECTNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 0.0 |
| | | IL-1beta | |
| Primary Th2 act | 0.0 | Microvascular Dermal EC none | 0.0 |
| Primary Tr1 act | 0.0 | Microsvasular Dermal EC | 0.0 |
| | | TNFalpha + IL-1beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium TNFalpha + | 0.0 |
| | | IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNFalpha + | 0.0 |
| act | | IL-1beta | |
| CD8 lymphocyte act | 0.0 | Astrocytes rest | 0.0 |
| Secondary CD8 lymphocyte | 0.0 | Astrocytes TNFalpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytcs) none | 0.0 |
| CH11 | | | |
| LAK cells rest | 0.0 | CCD1106 (Keratinocytcs) | 0.0 |
| | | TNFalpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 100.0 | NCI-H292 IL-4 | 0.0 |
| LAK cells PMA/ionomycin | 0.0 | NCI-H292 IL-9 | 0.1 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 | 0.0 |
| | | beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 1.1 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 rest | 0.0 |
| IL-4 | | | |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL-1 | 0.0 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 0.1 |
| HUVEC none | 0.0 | Kidney | 0.0 |
| HUVEC starved | 0.0 | | |

TABLE OH
Panel 5D

| | Rel. Exp. (%) | | Rel. Exp. (%) |
|---|---|---|---|
| | Ag2842, Run | | Ag2842, Run |
| Tissue Name | 223784832 | Tissue Name | 223784832 |
| 97457_Patient-02go_adipose | 0.0 | 94709_Donor 2 AM - A_adipose | 0.0 |
| 97476_Patient-07sk_skeletal | 0.0 | 94710_Donor 2 AM - B_adipose | 0.0 |
| muscle | | | |
| 97477_Patient-07ut_uterus | 0.0 | 94711_Donor 2 AM - C_adipose | 0.0 |
| 97478_Patient-07pl_placcnta | 0.0 | 94712_Donor 2 AD - A_adipose | 0.0 |
| 97481_Patient-08sk_skeletal | 0.0 | 947l3_Donor 2 AD - B_adipose | 0.0 |
| muscle | | | |
| 97482_Patient-08ut_uterus | 0.0 | 94714_Donor 2 AD - C_adipose | 29.9 |
| 97483_Patient-08pl_placenta | 0.0 | 94742_Donor 3 U - A_Mesenchymal | 0.0 |
| | | Stem Cells | |
| 97486_Patient-09sk_skeletal | 0.0 | 94743_Donor 3 U - B_Mesenchymal | 0.0 |
| muscle | | Stem Cells | |
| 97487_Patient-09ut_uterus | 0.0 | 94730_Donor 3 AM - A_adipose | 0.0 |
| 97488_Patient-09pl_placenta | 0.0 | 94731_Donor 3 AM - B_adipose | 0.0 |
| 97492_Patient-10ut_uterus | 25.9 | 94732_Donor 3 AM - C_adipose | 0.0 |
| 97493_Patient-10pl_placenta | 0.0 | 94733_Donor 3 AD - A_adipose | 0.0 |
| 97495_Patient-11go_adipose | 0.0 | 94734_Donor 3 AD - B_adipose | 0.0 |
| 97496_Patient-11sk_skeletal | 0.0 | 94735_Donor 3 AD - C_adipose | 0.0 |
| muscle | | | |
| 97497_Patient-11ut_uterus | 0.0 | 77138_Liver_HepG2untreated | 0.0 |
| 97498_Patient-11pl_placenta | 0.0 | 73556_Heart_Cardiac stromal cells | 0.0 |
| | | (primary) | |
| 97500_Patient-12go_adipose | 0.0 | 81735_Small Intestine | 0.0 |
| 97501_Patient-12sk_skeletal | 100.0 | 72409_Kidney_Proximal Convoluted | 0.0 |
| muscle | | Tubule | |
| 97502_Patient-12ut_uterus | 0.0 | 82685_Small intestine_Duodenum | 33.4 |
| 97503_Patient-12pl_placenta | 0.0 | 90650_Adrenal_Adrenocortical | 0.0 |
| | | adenoma | |
| 94721_Donor 2 U- | 0.0 | 72410_Kidney_HRCE | 0.0 |
| A Mesenchymal Stem Cells | | | |
| 94722_Donor 2 U- | 0.0 | 72411_Kidney_HRE | 0.0 |
| B Mesenchymal Stem Cells | | | |
| 94723_Donor 2 U- | 0.0 | 73139_Uterus_Uterine smooth muscle | 0.0 |
| C_Mesenchymal Stem Cells | | cells | |

General_screening_panel_v1.4 Summary: Ag4424 Expression of the CG55914-01 gene is highest in ovarian cancer cell line OVCAR-3 (CT=29.5). Therefore, expression of this gene can be used to distinguish this cell line from the other samples on this panel. In addition, there is low but significant expression of this gene associated with an additional ovarian cancer and two lung cancer cell lines. Therefore, therapeutic modulation of the activity of this gene or its protein product, through the use of small molecule drugs, protein therapeutics or antibodies, might be beneficial in the treatment of lung cancer or ovarian cancer.

Panel 1.3D Summary: Ag2842 Results from two experiments using the same probe/primer pair gave similar results. Expression of the CG55914-01 gene is highest in ovarian and lung cancer cell lines, consistent with what is observed in General_screening_panel_v 1.4.

Panel 2D Summary: Ag2842 Expression of this gene is highest in an ovarian cancer sample (CT=30.5) and is significantly higher than in the matched normal ovarian tissue. In addition, this gene is expressed at significant levels in another ovarian tumor. Therefore, expression of this gene may be used to distinguish ovarian cancers from the other samples on this panel. Furthermore, therapeutic modulation of the activity of the protein encoded by this gene may be beneficial in the treatment of ovarian cancer.

Panel 3D Summary: Ag2842 This gene is expressed at low levels in a number of cancer cell lines including fibrosarcoma, lung cancer and pancreatic ductal adenocarcinomiia. Therefore, expression of this gene may play a role in these types of cancers.

Panel 4D Summary: Ag2842 Expression of the CG55914-01 gene is highest in stimulated lymphokine-activated killer-(LAK) cells (CT=24.3). Therefore, expression of this gene can be used to distinguish this sample from the other samples on this panel. Since LAK cells are involved in tumor immunology and tumor cell clearance, as well as virally and bacterial infected cells, therapeutic modulation of this gene product may alter the functions of these cells and lead to improvement in cancer cell killing as well as host immunity to microbial and viral infections.

Panel 5D Summary: Ag2842 Results from one experiment with the CG55914-01 gene are not included. The amp plot indicates that there were experimental difficulties with this run.

NOV18

Expression of NOV18/CG57328-01 was assessed using the primer-probe set Ag3200, described in Table PA. Results of the RTQ-PCR runs are shown in Table PB.

TABLE PA
Probe Name Ag3200

| Primers | Sequences | Length | Start Position | SEQ ID NO |
|---|---|---|---|---|
| Forward | 5′-acaaccctacgtggatcattg-3′ | 21 | 251 | 313 |
| Probe | TET-5′-ccctattgatggaacaactaagtttgtcca-3′-TAMRA | 30 | 273 | 314 |
| Reverse | 5′-tcgaaacagctacaaaaggaaa-3′ | 22 | 307 | 315 |

TABLE PB
Panel 1.3D

| | Rel. Exp. (%) | | Rel. Exp. (%) |
|---|---|---|---|
| | Ag3200, Run | | Ag3200, Run |
| Tissue Name | 167994644 | Tissue Name | 167994644 |
| Liver adcnocarcinoma | 0.0 | Kidney (fetal) | 0.6 |
| Pancreas | 0.0 | Renal ca. 786-0 | 1.4 |
| Pancreatic ca. CAPAN2 | 0.0 | Renal ca. A498 | 4.0 |
| Adrenal gland | 0.0 | Renal ca. RXF 393 | 0.5 |
| Thyroid | 0.8 | Renal ca. ACHN | 0.0 |
| Salivary gland | 0.0 | Renal ca. UO-31 | 0.0 |
| Pituitary gland | 12.0 | Renal ca. TK-10 | 7.9 |
| Brain (fetal) | 6.6 | Liver | 0.9 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 2.7 | Lung | 4.9 |
| Brain (hippocampus) | 0.0 | Lung (fetal) | 2.0 |
| Brain (substantia nigra) | 2.1 | Lung ca. (small cell) LX-1 | 0.0 |
| Brain (thalamus) | 0.0 | Lung ca. (small cell) NCI- | 10.2 |
| | | H69 | |
| Cerebral Cortex | 0.0 | Lung ca. (s. cell var.) SHP- | 54.3 |
| | | 77 | |
| Spinal cord | 0.0 | Lung ca. (large cell) NCI- | 0.0 |
| | | H460 | |
| glio/astro U87-MG | 0.0 | Lung ca. (non-sm. cell) | 0.4 |
| | | A549 | |
| glio/astro U-118-MG | 0.0 | Lung ca. (non-s. cell) NCI- | 0.4 |
| | | H23 | |
| astrocytoma SW1783 | 0.0 | Lung ca. (non-s. cell) HOP- | 0.0 |
| | | 62 | |
| neuro*; met SK-N-AS | 1.1 | Lung ca. (non-s. cl) NCI- | 0.0 |
| | | H522 | |
| astrocytoma SF-539 | 0.0 | Lung ca. (squam.) SW 900 | 0.0 |
| astrocytoma SNB-75 | 0.0 | Lung ca. (squam.) NCI- | 100.0 |
| | | H596 | |
| glioma SNB-19 | 0.0 | Mammary gland | 0.0 |
| glioma U251 | 2.1 | Breast ca.* (pl. ef) MCF-7 | 0.7 |
| glioma SF-295 | 0.0 | Breast ca.* (pl. ef) MDA- | 0.0 |
| | | MB-231 | |
| Heart (fetal) | 0.0 | Breast ca.* (pl. ef) T47D | 2.0 |
| Heart | 6.4 | Breast ca. BT-549 | 0.0 |
| Skeletal muscle (fetal) | 0.0 | Breast ca. MDA-N | 0.9 |
| Skeletal muscle | 0.0 | Ovary | 0.0 |
| Bone marrow | 1.2 | Ovarian ca. OVCAR-3 | 1.1 |
| Thymus | 1.5 | Ovarian ca. OVCAR-4 | 0.0 |
| Spleen | 0.0 | Ovarian ca. OVCAR-5 | 1.6 |
| Lymph node | 1.8 | Ovarian ca. OVCAR-8 | 0.0 |
| Colorectal | 2.5 | Ovarian ca. IGROV-1 | 8.0 |
| Stomach | 0.0 | Ovarian ca.* (ascites) SK- | 16.0 |
| | | OV-3 | |
| Small intestine | 2.0 | Uterus | 0.0 |
| Colon ca. SW480 | 0.0 | Placenta | 0.0 |
| Colon ca.* SW620 (SW480 | 3.1 | Prostate | 1.9 |
| met) | | | |
| Colon ca. HT29 | 1.6 | Prostate ca.* (bone met) PC- | 2.3 |
| | | 3 | |
| Colon ca.HCT-116 | 2.0 | Testis | 1.9 |
| Colon ca. CaCo-2 | 5.0 | Melanoma Hs688(A).T | 0.0 |
| Colon ca. tissue (ODO3866) | 0.7 | Melanoma* (met) | 0.0 |
| | | Hs688(B).T | |
| Colon ca. HCC-2998 | 7.8 | Melanoma UACC-62 | 0.0 |
| Gastric ca.* (liver met) NCI- | 8.9 | Melanoma M14 | 0.0 |
| N87 | | | |
| Bladder | 3.1 | Melanoma LOX IMVI | 0.0 |
| Trachea | 2.2 | Melanoma* (met) SK- | 0.4 |
| | | MEL-5 | |
| Kidney | 0.0 | Adipose | 2.3 |

Panel 1.3D Summary: Ag3200 Significant expression of the CG57328-01 gene is restricted to two lung cancer cell lines (CT=32-33). Thus, expression of this gene could be used to distinguish these samples from the other samples in the panel. In addition, therapeutic modulation of the activity of this gene or its protein product, through the use of small molecule drugs, protein therapeutics or antibodies, might be beneficial in the treatment of lung cancer.

NOV19

Expression of NOV19 CG57358-01 was assessed using the primer-probe set Ag3214, described in Table QA. Results of the RTQ-PCR runs are shown in Tables QB, QC, QD and QE.

TABLE QA
Probe Name Ag3214

| Primers | Sequences | Length | Start Position | SEQ ID NO |
|---|---|---|---|---|
| Forward | 5′-ttcaacaggaaggtgattctca-3′ | 22 | 520 | 316 |
| Probe | TET-5′-attttcttctggtcggccgtcacctt-3′-TAMRA | 26 | 550 | 317 |
| Reverse | 5′-aagtactgctggggaatgaag-3′ | 21 | 585 | 318 |

TABLE QB
CNS_neurodegeneration_v1.0

| | Rel. Exp. (%) | | Rel. Exp. (%) |
|---|---|---|---|
| | Ag3214, Run | | Ag3214, Run |
| Tissue Name | 209861781 | Tissue Name | 209861781 |
| AD 1 Hippo | 19.5 | Control (Path) 3 Temporal | 5.4 |
| | | Ctx | |
| AD 2 Hippo | 23.5 | Control (Path) 4 Temporal | 25.9 |
| | | Ctx | |
| AD 3 Hippo | 14.8 | AD 1 Occipital Ctx | 19.3 |
| AD 4 Hippo | 8.5 | AD 2 Occipital Ctx | 0.0 |
| | | (Missing) | |
| AD 5 Hippo | 100.0 | AD 3 Occipital Ctx | 9.3 |
| AD 6 Hippo | 48.0 | AD 4 Occipital Ctx | 20.2 |
| Control 2 Hippo | 36.6 | AD 5 Occipital Ctx | 50.0 |
| Control 4 Hippo | 10.3 | AD 6 Occipital Ctx | 30.6 |
| Control (Path) 3 Hippo | 10.3 | Control 1 Occipital Ctx | 8.6 |
| AD 1 Temporal Ctx | 24.7 | Control 2 Occipital Ctx | 68.8 |
| AD 2 Temporal Ctx | 28.7 | Control 3 Occipital Ctx | 27.0 |
| AD 3 Temporal Ctx | 8.7 | Control 4 Occipital Ctx | 11.6 |
| AD 4 Temporal Ctx | 23.7 | Control (Path) 1 Occipital | 81.8 |
| | | Ctx | |
| AD 5 Inf Temporal Ctx | 95.3 | Control (Path) 2 Occipital | 14.8 |
| | | Ctx | |
| AD 5 Sup Temporal Ctx | 55.9 | Control (Path) 3 Occipital | 7.0 |
| | | Ctx | |
| AD 6 Inf Temporal Ctx | 52.9 | Control (Path) 4 Occipital | 17.0 |
| | | Ctx | |
| AD 6 Sup Temporal Ctx | 46.3 | Control 1 Parietal Ctx | 13.2 |
| Control 1 Temporal Ctx | 7.9 | Control 2 Parietal Ctx | 33.7 |
| Control 2 Temporal Ctx | 52.5 | Control 3 Parietal Ctx | 18.8 |
| Control 3 Temporal Ctx | 18.8 | Control (Path) 1 Parietal | 68.3 |
| | | Ctx | |
| Control 3 Temporal Ctx | 9.0 | Control (Path) 2 Parietal | 21.0 |
| | | Ctx | |
| Control (Path) 1 Temporal | 52.1 | Control (Path) 3 Parietal | 5.6 |
| Ctx | | Ctx | |
| Control (Path) 2 Temporal | 26.6 | Control (Path) 4 Parietal | 38.2 |
| Ctx | | Ctx | |

TABLE QC
Panel 1.3D

| | Rel. Exp. (%) | | Rel. Exp. (%) |
|---|---|---|---|
| | Ag3214, Run | | Ag3214, Run |
| Tissue Name | 168012858 | Tissue Name | 168012858 |
| Liver adenocarcinoma | 0.4 | Kidney (fetal) | 18.8 |
| Pancreas | 3.1 | Renal ca. 786-0 | 4.3 |
| Pancreatic ca. CAPAN2 | 5.8 | Renal ca. A498 | 0.6 |
| Adrenal gland | 0.8 | Renal ca. RXF 393 | 24.1 |
| Thyroid | 0.7 | Renal ca. ACHN | 75.3 |
| Salivary gland | 3.5 | Renal ca. UO-31 | 3.0 |
| Pituitary gland | 0.7 | Renal ca. TK-10 | 3.2 |
| Brain (fetal) | 7.3 | Liver | 1.5 |
| Brain (whole) | 21.9 | Liver (fetal) | 1.1 |
| Brain (amygdala) | 14.8 | Liver ca. (hepatoblast) | 0.0 |
| | | HepG2 | |
| Brain (cerebellum) | 9.0 | Lung | 16.6 |
| Brain (hippocampus) | 10.8 | Lung (fetal) | 14.3 |
| Brain (substantia nigra) | 12.9 | Lung ca. (small cell) LX-1 | 78.5 |
| Brain (thalamus) | 14.4 | Lung ca. (small cell) NCI- | 1.5 |
| | | H69 | |
| Cerebral Cortex | 24.5 | Lung ca. (s. cell var.) SHP- | 10.8 |
| | | 77 | |
| Spinal cord | 6.7 | Lung ca. (large cell) NCI- | 0.2 |
| | | H460 | |
| glio/astro U87-MG | 0.0 | Lung ca. (non-sm. cell) | 0.4 |
| | | A549 | |
| glio/astro U-118-MG | 0.9 | Lung ca. (non-s. cell) NCI- | 0.1 |
| | | H23 | |
| astrocytoma SW 1783 | 0.1 | Lung ca. (non-s. cell) HOP- | 0.1 |
| | | 62 | |
| neuro*; met SK-N-AS | 0.1 | Lung ca. (non-s. cl) NCI- | 0.2 |
| | | H522 | |
| astrocytoma SF-539 | 0.9 | Lung ca. (squam.) SW 900 | 8.4 |
| astrocytoma SNB-75 | 4.7 | Lung ca. (squam.) NCI- | 0.9 |
| | | H596 | |
| glioma SNB-19 | 0.1 | Mammary gland | 4.7 |
| glioma U251 | 0.1 | Breast ca.* (pl. ef) MCF-7 | 1.3 |
| glioma SF-295 | 0.1 | Breast ca.* (pl. ef) MDA- | 4.5 |
| | | MB-231 | |
| Heart (fetal) | 13.3 | Breast ca.* (pl. ef) T47D | 6.6 |
| Heart | 3.6 | Breast ca. BT-549 | 0.1 |
| Skeletal muscle (fetal) | 6.2 | Breast ca. MDA-N | 0.2 |
| Skeletal muscle | 1.5 | Ovary | 1.3 |
| Bone marrow | 1.7 | Ovarian ca. OVCAR-3 | 0.0 |
| Thymus | 1.3 | Ovarian ca. OVCAR-4 | 13.3 |
| Spleen | 3.0 | Ovarian ca. OVCAR-5 | 98.6 |
| Lymph node | 5.0 | Ovarian ca. OVCAR-8 | 0.2 |
| Colorectal | 1.0 | Ovarian ca. IGROV-1 | 8.4 |
| Stomach | 3.3 | Ovarian ca.* (ascitcs) SK- | 0.1 |
| | | OV-3 | |
| Small intestine | 3.8 | Uterus | 3.1 |
| Colon ca. SW480 | 3.6 | Placenta | 1.0 |
| Colon ca.* SW620 (SW480 | 100.0 | Prostate | 0.6 |
| met) | | | |
| Colon ca. HT29 | 13.0 | Prostate ca.* (bone met) PC- | 0.0 |
| | | 3 | |
| Colon ca. HCT-116 | 02 | Testis | 0.6 |
| Colon ca. CaCo-2 | 0.1 | Melanoma Hs688(A).T | 0.0 |
| Colon ca tissue(ODO3866) | 16.2 | Melanoma* (met) | 0.0 |
| | | Hs688(B).T | |
| Colon ca. HCC-2998 | 28.9 | Melanoma UACC-62 | 0.1 |
| Gastric ca.* (liver met) NCI- | 6.2 | Melanoma M14 | 0.0 |
| N87 | | | |
| Bladder | 11.0 | Melanoma LOX IMVI | 0.0 |
| Trachea | 2.1 | Melanoma* (met) SK- | 0.2 |
| | | MEL-5 | |
| Kidney | 32.5 | Adipose | 3.9 |

TABLE QD
Panel 2.2

| | Rel. Exp. (%) | | Rel. Exp. (%) |
|---|---|---|---|
| | Ag3214, Run | | Ag3214, Run |
| Tissue Name | 174416265 | Tissue Name | 174416265 |
| Normal Colon | 1.8 | Kidney Margin (OD04348) | 62.9 |
| Colon cancer (OD06064) | 37.6 | Kidney malignant cancer | 0.8 |
| | | (OD06204B) | |
| Colon Margin (OD06064) | 2.8 | Kidney normal adjacent tissue | 26.4 |
| | | (OD06204E) | |
| Colon cancer (OD06159) | 1.1 | Kidney Cancer (OD04450-01) | 33.9 |
| Colon Margin (OD06159) | 0.8 | Kidney Margin (OD04450-03) | 15.7 |
| Colon cancer (OD06297-04) | 1.9 | Kidney Cancer 8120613 | 0.4 |
| Colon Margin (OD06297-05) | 2.1 | Kidney Margin 8120614 | 58.6 |
| CC Gr 2 ascend colon | 5.3 | Kidney Cancer 9010320 | 3.5 |
| (ODO3921) | | | |
| CC Margin (ODO3921) | 0.6 | Kidney Margin 9010321 | 20.2 |
| Colon cancer metastasis | 3.4 | Kidney Cancer 8120607 | 100.0 |
| (OD06104) | | | |
| Lung Margin (OD06104) | 3.2 | Kidney Margin 8120608 | 47.6 |
| Colon mets to lung (OD04451- | 6.8 | Normal Uterus | 2.8 |
| 01) | | | |
| Lung Margin (OD04451-02) | 5.0 | Unterine Cancer 064011 | 1.1 |
| Normal Prostate | 0.4 | Normal Thyroid | 0.5 |
| Prostate Cancer (OD04410) | 1.1 | Thyroid Cancer 064010 | 0.1 |
| Prostate Margin (OD04410) | 1.2 | Thyroid Cancer A302152 | 2.0 |
| Normal Ovary | 1.4 | Thyroid Margin A302153 | 0.7 |
| Ovarian cancer (OD06283-03) | 0.3 | Normal Breast | 1.7 |
| Ovarian Margin (OD06283-07) | 8.0 | Breast Cancer (OD04566) | 0.8 |
| Ovarian Cancer 064008 | 1.7 | Breast Cancer 1024 | 6.9 |
| Ovarian cancer (OD06145) | 1.1 | Breast Cancer (OD0459001) | 3.0 |
| Ovarian Margin (OD06145) | 2.2 | Breast Cancer Mets (OD04590- | 1.6 |
| | | 03) | |
| Ovarian cancer (OD06455-03) | 6.5 | Breast Cancer Metastasis | 2.9 |
| | | (OD04655-05) | |
| Ovarian Margin (OD06455-07) | 1.3 | Breast Cancer 064006 | 2.3 |
| Normal Lung | 5.7 | Breast Cancer 9100266 | 1.4 |
| Invasive poor diff, lung adeno | 0.2 | Breast Margin 9100265 | 1.1 |
| (ODO4945-01 | | | |
| Lung Margin (ODO4945-03) | 6.2 | Breast Cancer A209073 | 1.9 |
| Lung Malignant Cancer | 15.6 | Breast Margin A2090734 | 2.1 |
| (OD03126) | | | |
| Lung Margin (OD03126) | 3.2 | Breast cancer (OD06083) | 11.9 |
| Lung Cancer (OD05014A) | 1.3 | Breast cancer node metastasis | 11.2 |
| | | (OD06083) | |
| Lung Margin (OD05014B) | 3.5 | Normal Liver | 1.9 |
| Lung cancer (OD06081) | 1.4 | Liver Cancer 1026 | 2.5 |
| Lung Margin (OD06081) | 4.9 | Liver Cancer 1025 | 3.1 |
| Lung Cancer (OD04237-01) | 1.0 | Liver Cancer 6004-T | 1.8 |
| Lung Margin (OD04237-02) | 12.3 | Liver Tissue 6004-N | 0.0 |
| Ocular Melanoma Metastasis | 0.0 | Liver Cancer 6005-T | 2.2 |
| Ocular Melanoma Margin | 1.0 | Liver Tissue 6005-N | 11.2 |
| (Liver) | | | |
| Melanoma Metastasis | 0.2 | Liver Cancer 064003 | 1.0 |
| Melanoma Margin (Lung) | 4.2 | Normal Bladder | 8.1 |
| Normal Kidney | 9.1 | Bladder Cancer 1023 | 4.1 |
| Kidney Ca, Nuclear grade 2 | 24.8 | Bladder Cancer A302173 | 0.6 |
| (OD04338) | | | |
| Kidney Margin (OD04338) | 11.7 | Normal Stomach | 3.8 |
| Kidney Ca Nuclear grade 1/2 | 5.1 | Gastric Cancer 9060397 | 15.2 |
| (OD04339) | | | |
| Kidney Margin (OD04339) | 23.0 | Stomach Margin 9060396 | 11.7 |
| Kidney Ca, Clear cell type | 14.1 | Gastric Cancer 9060395 | 7.1 |
| (OD04340) | | | |
| Kidney Margin (OD04340) | 16.6 | Stomach Margin 9060394 | 17.2 |
| Kidney Ca, Nuclear grade 3 | 0.7 | Gastric Cancer 064005 | 3.7 |
| (OD04348) | | | |

TABLE QE
Panel 4D

| | Rel. Exp. (%) | | Rel. Exp. (%) |
|---|---|---|---|
| | Ag3214, Run | | Ag3214, Run |
| Tissue Name | 164682510 | Tissue Name | 164682510 |
| Secondary Th1 act | 0.3 | HUVEC IL-1beta | 3.5 |
| Secondary Th2 act | 0.2 | HUVBC IFN gamma | 14.4 |
| Secondary Tr1 act | 0.6 | HUVEC TNF alpha + IFN gamma | 6.0 |
| Secondary Th1 rest | 0.5 | HUVEC TNF alpha + IL4 | 5.8 |
| Secondary Th2 rest | 1.4 | HUVEC IL-11 | 24.8 |
| Secondary Tr1 rest | 1.1 | Lung Microvascular EC none | 50.0 |
| Primary Th1 act | 0.2 | Lung Microvascular EC TNFalpha | 40.6 |
| | | + IL-1beta | |
| Primary Th2 act | 0.1 | Microvascular Dermal EC none | 32.1 |
| Primary Tr1 act | 0.0 | Microsvasular Dermal EC | 24.8 |
| | | TNFalpha + IL-1beta | |
| Primary Th1 rest | 0.8 | Bronchial epithelium TNFalpha + | 3.0 |
| | | IL1beta | |
| Primary Th2 rest | 0.7 | Small airway epithelium none | 9.0 |
| Primary Tr1 rest | 1.6 | Small airway epithelium TNFalpha | 19.6 |
| | | + IL-1beta | |
| CD45RA CD4 lymphocyte | 0.1 | Coronery artery SMC rest | 0.2 |
| act | | | |
| CD45RO CD4 lymphocyte | 0.3 | Coronery artery SMC TNFalpha + | 0.0 |
| act | | IL-1beta | |
| CD8 lymphocyte act | 0.1 | Astrocytes rest | 3.5 |
| Secondary CD8 lymphocyte | 0.0 | Astrocytes TNFalpha + IL-1beta | 5.0 |
| rest | | | |
| Secondary CD8 lymphocyte | 0.0 | KU-812 (Basophil) rest | 0.6 |
| act | | | |
| CD4 lymphocyte none | 0.4 | KU-812 (Basophil) | 0.4 |
| | | PMA/ionomycin | |
| 2ry Th1/Th2/Tr1_anti-CD95 | 0.9 | CCD1106 (Keratinocytes) none | 0.8 |
| CH11 | | | |
| LAK cells rest | 0.1 | CCD1106 (Keratinocytes) | 0.1 |
| | | TNFalpha + IL-1beta | |
| LAK cells IL-2 | 0.4 | Liver cirrhosis | 6.3 |
| LAK cells IL-2 + IL-12 | 0.2 | Lupus kidney | 6.1 |
| LAK cells IL-2 + IFN gamma | 0.2 | NCI-H292 none | 1.3 |
| LAK cells IL-2 + IL-18 | 0.0 | NCI-H292 IL-4 | 1.0 |
| LAK cells PMA/ionomycin | 0.0 | NCI-H292 IL-9 | 2.2 |
| NK Cells IL-2 rest | 0.2 | NCI-H292 IL-I3 | 0.7 |
| Two Way MLR 3 day | 0.0 | NCI-H292 IFN gamma | 2.3 |
| Two Way MLR 5 day | 0.3 | HPAEC none | 15.4 |
| Two Way MLR 7 day | 0.0 | HPAEC TNF alpha + IL-1 beta | 2.1 |
| PBMC rest | 0.1 | Lung fibroblast none | 0.0 |
| PBMC PWM | 0.3 | Lung fibroblast TNF alpha + IL-1 | 0.1 |
| | | beta | |
| PBMC PHA-L | 0.6 | Lung fibroblast IL-4 | 0.3 |
| Ramos (B cell) none | 0.0 | Lung fibroblast IL-9 | 0.0 |
| Ramos (B cell) ionomycin | 0.2 | Lung fibroblast IL-13 | 0.0 |
| B lymphocytes PWM | 0.1 | Lung fibroblast IFN gamma | 0.1 |
| B lymphocytes CD40L and | 0.2 | Dermal fibroblast CCD1070 rest | 0.0 |
| IL-4 | | | |
| EOL-1 dbcAMP | 3.5 | Dermal fibroblast CCD1070 TNF | 2.9 |
| | | alpha | |
| EOL-1 dbcAMP | 6.6 | Dermal fibroblast CCD1070 IL-1 | 0.0 |
| PMA/ionomycin | | beta | |
| Dendritic cells none | 0.1 | Dermal fibroblast IFN gamma | 0.0 |
| Dendritic cells LPS | 0.2 | Dermal fibroblast IL-4 | 0.0 |
| Dendritic cells anti-CD40 | 0.2 | IBD Colitis 2 | 0.3 |
| Monocytes rest | 0.5 | IBD Crohn's | 0.9 |
| Monocytes LPS | 0.1 | Colon | 9.1 |
| Macrophages rest | 0.1 | Lung | 20.0 |
| Macrophages LPS | 0.0 | Thymus | 100.0 |
| HUVEC none | 12.1 | Kidney | 5.8 |
| HUVEC starved | 23.5 | | |

CNS_neurodegeneration_v1.0 Summary: Ag3214 This panel confirms the expression of this gene at low levels in the brain in an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag3214 Expression of the CG57358-01 gene is highest in colon cancer cell line SW620 and an ovarian cancer cell line (CTs=26.1). It is expressed in a number of cancer cell lines including ovarian, breast, lung, renal, liver adenocarcinoma, and astrocytoma. Therefore, this gene may play a role in these types of cancer.

This gene is expressed at moderate levels throughout the CNS, including in amygdala, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression. The CG57358-01 gene encodes a protein with similarity to the Drosophila spinster gene. Expression of the CG57358-01 gene in brain is consistent with observations made on the Drosophila spinster gene. Mutations of the spinster gene in Drosophila cause degeneration of adults neurons and reductions in programmed neuronal cell death (ref. 1). Thus, modulation of expression of the CG57358-01 gene may be important in the treatment of neurodegenerative diseases and aging.

Among tissues with metabolic or endocrine function, this gene is expressed at moderate levels in adipose, pancreas, liver, skeletal muscle, and heart and at low levels in thyroid, adrenal gland and pituitary gland. Therefore, therapeutic modulation of the activity of this gene may prove useful in the treatment of endocrine/metabolically related diseases, such as obesity and diabetes.

References:

1. Nakano Y, Fujitani K, Kurihara J, Ragan J, Usui-Aoki K, Shimoda L, Lukacsovich T, Suzuki K, Sezaki M, Sano Y, Ueda R, Awano W, Kaneda M, Umeda M, Yamamoto D. Mutations in the novel membrane protein spinster interfere with programmed cell death and cause neural degeneration in Drosophila melanogaster. Mol Cell Biol 2001 June;21(11):3775-88.

Mutations in the spin gene are characterized by an extraordinarily strong rejection behavior of female flies in response to male courtship. They are also accompanied by decreases in the viability, adult life span, and oviposition rate of the flies. In spin mutants, some oocytes and adult neural cells undergo degeneration, which is preceded by reductions in programmed cell death of nurse cells in ovaries and of neurons in the pupal nervous system, respectively. The central nervous system (CNS) of spin mutant flies accumulates autofluorescent lipopigments with characteristics similar to those of lipofuscin. The spin locus generates at least five different transcripts, with only two of these being able to rescue the spin behavioral phenotype; each encodes a protein with multiple membrane-spanning domains that are expressed in both the surface glial cells in the CNS and the follicle cells in the ovaries. Orthologs of the spin gene have also been identified in a number of species from nematodes to humans. Analysis of the spin mutant will give us new insights into neurodegenerative diseases and aging.

PMID: 11340170

Panel 2.2 Summary: Ag3214 Expression of the CG57358-01 gene is highest in a kidney cancer sample (CT=28.4). However, in general expression of this gene appears to be higher in matched normal kidney and lung tissues when compared to the adjacent tumors. Therefore, therapeutic modulation of the activity of this gene or its protein product, using small Molecule drugs, antibodies or protein therapeutics, may be of use in the treatment of kidney and lung cancer.

Panel 4D Summary: Ag3214 Expression of the CG57358-01 gene is highest in thymus (CT=27). In addition, low levels of expression of this gene are detected in resting T cells. Therefore, small molecule therapeutics or antibody therapeutics designed against the protein encoded for by this gene could be utilized to modulate immune function (T cell development) and be important for organ transplant, AIDS treatment or post chemotherapy immune reconstitiution.

Expression of this gene is also detected at moderate levels in a number of endothelial cell samples on this panel including lung microvascular endothelial cells, microvascular dermal endothelial cells, HPAEC and HUVECs, suggesting a role for this gene in endothelium integrity or homeostasis. Thus, this gene may play a role in inflammation. Therefore, therapeutic modulation of the activity of this gene, using small molecule drugs, antibodies or protein therapeutics, may be of use in the treatment or prevention of inflammatory reactions.

NOV20

Expression of NOV20/CG57695-01 was assessed using the primer-probe set Ag3310, described in Table RA. Results of the RTQ-PCR run are shown in Tables RB.

TABLE RA
Probe Name Ag3310

| Primers | Sequences | Length | Start Position | SEQ ID NO |
|---|---|---|---|---|
| Forward | 5′-gaacttcctcccatttcttcag-3′ | 22 | 330 | 319 |
| Probe | TET-5′-aaacccaggagatcagcataagacaa-3′-TAMRA | 26 | 378 | 320 |
| Reverse | 5′-tagtgggaacaaaggggtagac-3′ | 22 | 406 | 321 |

TABLE RB
General_screening_panel_v1.4

| | Rel. Exp. (%) | | Rel. Exp. (%) |
|---|---|---|---|
| | Ag3310, Run | | Ag3310, Run |
| Tissue Name | 215642635 | Tissue Name | 215642635 |
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI- | 0.0 |
| | | N87 | |
| Melanoma* M14 | 0.0 | Gastric ca. KATO III | 7.5 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell carcinoma | 0.0 | Colon ca.* (SW480 met) | 0.0 |
| SCC-4 | | SW620 | |
| Testis Pool | 100.0 | Colon ca. HT29 | 0.0 |
| Prostate ca.* (bone met) | 0.0 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 5.4 |
| Ovarian ca. OVCAR-3 | 0.0 | Colon ca. Colo-205 | 0.0 |
| Ovarian ca. SK-OV-3 | 4.5 | Colon ca. SW-48 | 0.0 |
| Ovarian ca. OVCAR-4 | 0.0 | Colon Pool | 11.9 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 10.5 | Fetal Skeletal Muscle | 0.0 |
| Breast ca. T47D | 0.0 | Skeletal Muscle Pool | 0.0 |
| Breast ca. MDA-N | 0.0 | Spleen Pool | 1.6 |
| Breast Pool | 0.0 | Thymus Pool | 0.0 |
| Trachea | 0.0 | CNS cancer (glio/astro) U87- | 0.0 |
| | | MG | |
| Lung | 17.7 | CNS cancer (glio/astro) U- | 0.0 |
| | | 118-MG | |
| Fetal Lung | 25.2 | CNS cancer (neuro;met) SK- | 0.0 |
| | | N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 1.6 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) Pool | 0.0 |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 7.5 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

CNS_neurodegeneration_v1.0 Summary: Ag3310 Expression of the CG57695-01 gene is low/undetectable (CTs>35) across all of the samples on this panel (data not shown).

General_screening_panel_v1.4 Summary: Ag3310 Expression of the CG57695-01 gene is seen only in the testis (CT=33.8) Therefore, expression of this gene can be used to distinguish testis from the other samples on this panel. In addition, therapeutic modulation of the activity of this gene may be of use in the treatment of reproductive disorders such as infertility.

Panel 4D Summary: Ag3310 Expression of the CG57695-01 gene is low/undetectable (CTs>35) across all of the samples on this panel (data not shown).

NOV21

Expression of NOV21/CG57654-01 was assessed using the primer-probe set Ag1680, described in Table SA. Results of the RTQ-PCR runs are shown in Tables SB, SC, SD and SE.

TABLE SA
Probe Name Ag1680

| Primers | Sequences | Length | Start Position |
|---|---|---|---|
| Forward | 5′-gccaattacaattgcacaattt-3′ | 22 | 795 |
| Probe | TET-5′-atgaacactcctgccccttggagtt-3′-TAMRA | 25 | 825 |
| Reverse | 5′-tcttcacgtggatagccataac-3′ | 22 | 855 |

TABLE SB
CNS_neurodegeneration_v1.0

| | Rel. Exp. (%) | | Rel. Exp. (%) |
|---|---|---|---|
| | Ag1680, Run | | Ag1680, Run |
| Tissue Name | 207624859 | Tissue Name | 207624859 |
| AD 1 Hippo | 5.0 | Control (Path) 3 Temporal | 0.8 |
| | | Ctx | |
| AD 2 Hippo | 9.0 | Control (Path) 4 Temporal | 20.4 |
| | | Ctx | |
| AD 3 Hippo | 2.9 | AD 1 Occipital Ctx | 12.9 |
| AD 4 Hippo | 3.7 | AD 2 Occipital Ctx | 0.0 |
| | | (Missing) | |
| AD 5 Hippo | 85.3 | AD 3 Occipital Ctx | 1.3 |
| AD 6 Hippo | 38.2 | AD 4 Occipital Ctx | 19.3 |
| Control 2 Hippo | 27.0 | AD 5 Occipital Ctx | 65.5 |
| Control 4 Hippo | 2.4 | AD 6 Occipital Ctx | 43.2 |
| Control (Path) 3 Hippo | 0.6 | Control 1 Occipital Ctx | 0.3 |
| AD 1 Temporal Ctx | 4.8 | Control 2 Occipital Ctx | 100.0 |
| AD 2 Temporal Ctx | 12.6 | Control 3 Occipital Ctx | 16.6 |
| AD 3 Temporal Ctx | 2.5 | Control 4 Occipital Ctx | 2.1 |
| AD 4 Temporal Ctx | 14.0 | Control (Path) 1 Occipital | 69.7 |
| | | Ctx | |
| AD 5 Inf Temporal Ctx | 68.3 | Control (Path) 2 Occipital | 12.8 |
| | | Ctx | |
| AD 5 Sup Temporal Ctx | 17.3 | Control (Path) 3 Occipital | 0.2 |
| | | Ctx | |
| AD 6 Inf Temporal Ctx | 47.6 | Control (Path) 4 Occipital | 10.2 |
| | | Ctx | |
| AD 6 Sup Temporal Ctx | 61.1 | Control 1 Parietal Ctx | 1.3 |
| Control 1 Temporal Ctx | 0.9 | Control 2 Parietal Ctx | 18.2 |
| Control 2 Temporal Ctx | 52.5 | Control 3 Parietal Ctx | 21.0 |
| Control 3 Temporal Ctx | 15.9 | Control (Path) 1 Parietal | 59.5 |
| | | Ctx | |
| Control 3 Temporal Ctx | 4.8 | Control (Path) 2 Parietal | 23.2 |
| | | Ctx | |
| Control (Path) 1 Temporal | 42.0 | Control (Path) 3 Parietal | 0.6 |
| Ctx | | Ctx | |
| Control (Path) 2 Temporal | 40.6 | Control (Path) 4 Parietal | 30.6 |
| Ctx | | Ctx | |

TABLE SC
Panel 1.3D

| | Rel. Exp. (%) | | Rel. Exp. (%) |
|---|---|---|---|
| | Ag1680, Run | | Ag1680, Run |
| Tissue Name | 158317296 | Tissue Name | 158317296 |
| Liver adenocarcinoma | 0.3 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.5 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 8.1 | Renal ca. TK-10 | 0.0 |
| Brain (fetal) | 3.7 | Liver | 0.0 |
| Brain (whole) | 27.0 | Liver (fetal) | 0.0 |
| Brain (amygdala) | 25.0 | Liver ca. (hepatoblast) | 0.0 |
| | | HepG2 | |
| Brain (cerebellum) | 17.4 | Lung | 0.0 |
| Brain (hippocampus) | 100.0 | Lung (fetal) | 0.0 |
| Brain (substantia nigra) | 2.4 | Lung ca. (small cell) LX-1 | 0.6 |
| Brain (thalamus) | 14.6 | Lung ca. (small cell) NCI- | 0.0 |
| | | H69 | |
| Cerebral Cortex | 83.5 | Lung ca. (s. cell var.) SHP- | 4.2 |
| | | 77 | |
| Spinal cord | 0.5 | Lung ca. (large cell) NCI- | 0.0 |
| | | H460 | |
| glio/astro U87-MG | 0.0 | Lung ca. (non-sm. cell) | 0.0 |
| | | A549 | |
| glio/astro U-118-MG | 0.0 | Lung ca. (non-s. cell) NCI- | 0.0 |
| | | H23 | |
| astrocytoma SW1783 | 0.0 | Lung ca. (non-s. cell) HOP- | 0.0 |
| | | 62 | |
| neuro*; met SK-N-AS | 4.0 | Lung ca. (non-s. cl) NCI- | 0.0 |
| | | H522 | |
| astrocytoma SF-539 | 0.0 | Lung ca. (squam.) SW 900 | 0.0 |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl. ef) MCF-7 | 0.0 |
| glioma SF-295 | 0.0 | Breast ca.* (pl. ef) MDA- | 0.0 |
| | | MB-231 | |
| Heart (fetal) | 0.0 | Breast ca.* (pl. ef) T47D | 0.5 |
| Heart | 0.0 | Breast ca. BT-549 | 0.0 |
| Skeletal muscle (fetal) | 0.0 | Breast ca. MDA-N | 1.3 |
| Skeletal muscle | 0.0 | Ovary | 0.0 |
| Bone marrow | 0.0 | Ovarian ca. OVCAR-3 | 0.3 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) SK- | 0.0 |
| | | OV-3 | |
| Small intestine | 0.2 | Uterus | 0.0 |
| Colon ca. SW480 | 0.0 | Placenta | 0.1 |
| Colon ca.* SW620(SW480 | 0.9 | Prostate | 0.0 |
| met) | | | |
| Colon ca. HT29 | 0.0 | Prostate ca.* (bone met) PC- | 0.0 |
| | | 3 | |
| Colon ca. HCT-116 | 0.7 | Testis | 0.1 |
| Colon ca. CaCo-2 | 0.0 | Melanoma Hs688(A).T | 0.0 |
| Colon ca. tissue(ODO3866) | 0.0 | Melanoma* (met) | 0.0 |
| | | Hs688(B).T | |
| Colon ca. HCC-2998 | 0.0 | Melanoma UACC-62 | 0.2 |
| Gastric ca.* (liver met) NCI- | 0.0 | Melanoma M14 | 0.0 |
| N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE SD
Panel 2D

| | Rel. Exp. (%) | | Rel. Exp. (%) |
|---|---|---|---|
| | Ag1680, Run | | Ag1680, Run |
| Tissue Nnme | 158318977 | Tissue Name | 158318977 |
| Normal Colon | 100.0 | Kidney Margin 8120608 | 0.0 |
| CC Well to Mod Diff(ODO3866) | 0.0 | Kidney Cancer 8120613 | 0.0 |
| CC Margin (ODO3866) | 8.7 | Kidney Margin 8120614 | 0.0 |
| CC Gr.2 rectosigmoid (ODO3868) | 0.0 | Kidney Cancer 9010320 | 85.3 |
| CC Margin (ODO3868) | 12.3 | Kidney Margin 9010321 | 0.0 |
| CC Mod Diff (ODO3920) | 7.5 | Normal Uterus | 0.0 |
| CC Margin (ODO3920) | 35.4 | Uterus Cancer 064011 | 0.0 |
| CC Gr.2 ascend colon (ODO3921) | 7.6 | Normal Thyroid | 0.0 |
| CC Margin (ODO3921) | 9.5 | Thyroid Cancer 064010 | 0.0 |
| CC from Partial Hepatectomy | 0.0 | Thyroid Cancer A302152 | 0.0 |
| (ODO4309) Mets | | | |
| Liver Margin (ODO4309) | 0.0 | Thyroid Margin A302153 | 0.0 |
| Colon mets to lung (OD04451-01) | 0.0 | Normal Breast | 0.0 |
| Lung Margin (OD04451-02) | 0.0 | Breast Cancer (OD04566) | 0.0 |
| Normal Prostate 6546-1 | 0.0 | Breast Cancer (OD04590-01) | 0.0 |
| Prostate Cancer (OD04410) | 0.0 | Breast Cancer Mets | 0.0 |
| | | (OD04590-03) | |
| Prostate Margin (OD04410) | 0.0 | Breast Cancer Metastasis | 7.7 |
| | | (OD04655-05) | |
| Prostate Cancer (OD04720-01) | 7.4 | Breast Cancer 064006 | 0.0 |
| Prostate Margin (OD04720-02) | 12.6 | Breast Cancer 1024 | 0.0 |
| Normal Lung 061010 | 7.9 | Breast Cancer 9100266 | 0.0 |
| Lung Met to Muscle (ODO4286) | 0.0 | Breast Margin 9100265 | 0.0 |
| Muscle Margin (ODO4286) | 0.0 | Breast Cancer A209073 | 0.0 |
| Lung Malignant Cancer | 0.0 | Breast Margin A209073 | 0.0 |
| (OD03126) | | | |
| Lung Margin (OD03126) | 0.0 | Normal Liver | 0.0 |
| Lung Cancer (OD04404) | 0.0 | Liver Cancer 064003 | 0.0 |
| Lung Margin (OD04404) | 0.0 | Liver Cancer 1025 | 0.0 |
| Lung Cancer (OD04565) | 26.8 | Liver Cancer 1026 | 0.0 |
| Lung Margin (OD04565) | 0.0 | Liver Cancer 6004-T | 0.0 |
| Lung Cancer (OD04237-01) | 0.0 | Liver Tissue 6004-N | 0.0 |
| Lung Margin (OD04237-02) | 0.0 | Liver Cancer 6005-T | 0.0 |
| Ocular Mel Met to Liver | 0.0 | Liver Tissue 6005-N | 0.0 |
| (ODO4310) | | | |
| Liver Margin (ODO4310) | 0.0 | Normal Bladder | 16.0 |
| Melanoma Mets to Lung | 0.0 | Bladder Cancer 1023 | 0.0 |
| (OD04321) | | | |
| Lung Margin (OD04321) | 0.0 | Bladder Cancer A302173 | 57.4 |
| Normal Kidney | 0.0 | Bladder Cancer (OD04718- | 0.0 |
| | | 01) | |
| Kidney Ca, Nuclear grade 2 | 0.0 | Bladder Normal Adjacent | 0.0 |
| (OD04338) | | (OD04718-03) | |
| Kidney Margin (OD04338) | 0.0 | Normal Ovary | 0.0 |
| Kidney Ca Nuclear grade 1/2 | 0.0 | Ovarian Cancer 064008 | 43.2 |
| (OD04339) | | | |
| Kidney Margin (OD04339) | 0.0 | Ovarian Cancer (OD04768- | 0.0 |
| | | 07) | |
| Kidney Ca, Clear cell type | 0.0 | Ovary Margin (OD04768-08) | 0.0 |
| (OD04340) | | | |
| Kidney Margin (OD04340) | 0.0 | Normal Stomach | 7.4 |
| Kidney Ca, Nuclear grade 3 | 0.0 | Gastric Cancer 9060358 | 0.0 |
| (OD04348) | | | |
| Kidney Margin (OD04348) | 0.0 | Stomach Margin 9060359 | 3.3 |
| Kidney Cancer (OD04622-01) | 0.0 | Gastric Cancer 9060395 | 8.1 |
| Kidney Margin (OD04622-03) | 0.0 | Stomach Margin 9060394 | 7.4 |
| Kidney Cancer (OD04450-01) | 0.0 | Gastric Cancer 9060397 | 1.3 |
| Kidney Margin (OD04450-03) | 0.0 | Stomach Margin 9060396 | 6.5 |
| Kidney Cancer 8120607 | 0.0 | Gastric Cancer 064005 | 0.0 |

TABLE SE
Panel 4D

| | Rel. Exp. (%) | | Rel. Exp. (%) |
|---|---|---|---|
| | Ag1680, Run | | Ag1680, Run |
| Tissue Name | 158320708 | Tissue Name | 158320708 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HIUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha | 0.0 |
| | | + IL-1beta | |
| Primary Th2 act | 0.0 | Microvascular Dermal EC none | 0.0 |
| Primary Tr1 act | 0.0 | Microsvasular Dermal EC | 0.0 |
| | | TNFalpha + IL-1beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium TNFalpha | 0.0 |
| | | + IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNFalpha + | 0.0 |
| act | | IL-1beta | |
| CD8 lymphocyte act | 0.0 | Astrocytes rest | 0.0 |
| Secondary CD8 lymphocyte | 0.0 | Astrocytes TNFalpha + IL-1beta | 0.0 |
| rest | | | |
| Secondary CD8 lymphocyte | 0.0 | KU-812 (Basophil) rest | 19.9 |
| act | | | |
| CD4 lymphocyte none | 0.0 | KU-812 (Basophil) | 48.3 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN gamma | 0.0 | NCI-H292 none | 94.0 |
| LAK cells IL-2 + IL-18 | 0.0 | NCI-H292 IL-4 | 31.2 |
| LAK cells PMA/ionomycin | 0.0 | NCI-H292 IL-9 | 100.0 |
| NK Cells IL-2 rest | 0.0 | NCI-H292 IL-13 | 16.4 |
| Two Way MLR 3 day | 0.0 | NCI-H292 IFN gamma | 20.2 |
| Two Way MLR 5 clay | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 | 0.0 |
| | | beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 rest | 0.0 |
| IL-4 | | | |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL-1 | 0.0 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 7.5 |
| Monocytes rest | 0.0 | IBD Crohn's | 36.1 |
| Monocytes LPS | 0.0 | Colon | 49.3 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag1680 This panel confirms the expression of this gene at moderate to high levels in the brain in an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag1680 The CG57654-01 gene is expressed at high to moderate levels in all regions of the CNS examined, including in amygdala, substantia nigra, thalamus, cerebellum, cerebral cortex, and spinal cord with the highest levels in the hippocampus (CT=26.3). Therefore, expression of this gene may be used to distinguish brain samples from the other samples on this panel. This gene encodes a novel splice variant of the gamma-aminobutyric-acid receptor gamma-2 subunit precursor (GABA-A Receptor) gene. Several drugs, including benzodiazepines, anticonvulsants, anaesthetics and neurosteroids interact with binding sites on GABA-A receptors (ref 1). Therefore, modulation of expression of this gene may be used for treatment of central nervous system disorders such as epilepsy, anxiety and alcoholism.

Among tissues with metabolic or endocrine function, this gene is expressed at low levels in adrenal gland and pituitary gland. Therefore, therapeutic modulation of the activity of this gene may prove useful in the treatment of endocrine/metabolically related diseases, such as obesity and diabetes.

References:

1. Smith T A. Type A gamma-aminobutyric acid (GABAA) receptor subunits and benzodiazepine binding: significance to clinical syndromes and their treatment. Br J Biomed Sci 2001;58(2):111-21.

Gamma (gamma)-aminobutyric acid (GABA) acting via GABAA receptors is the brain's major inhibitory neurotransmitter system and exerts a crucial role in regulating brain excitability. A number of drugs interact with binding sites on GABAA receptors, and these include benzodiazepines, anticonvulsants, anaesthetics and neurosteroids (e.g. the progesterone metabolite pregnalone). GABAA receptors comprise five subunits (19 are known currently), and are classified into three major groups (alpha, beta and gamma) and several minor ones. The subunit make-up of a receptor, particularly its alpha-subunit content, determines its pharmacological characteristics. Thus, receptors that include an alpha 1 subunit have a benzodiazepine (BZ) type I (BZ[I]) pharmacology and bind zolpidem and CL218,872 with high affinity, whilst receptors with alpha2, alpha3 or alpha5 subunits have a BZ type II (BZ[II]) pharmacology and bind these drugs with low affinity. In contrast to receptors that contain alpha4 and alpha6 subunits, which are diazepam-insensitive, both BZ(I) and -(II) bind diazepam and other benzodiazepines. The ligand selectivity of receptor subunits assists in their characterisation. Using immunochemical and ligand-binding techniques, the subunit composition of GABAA receptors has been shown to exhibit a degree of brain regional specificity. GABAA receptors are of great clinical significance in several disorders, including epilepsy, anxiety and alcoholism. In addition to treating epilepsy with drugs that target GABAA and BZ binding sites, epileptic lesions can be localised presurgically using radiolabelled BZ ligands. BZs are used commonly to treat anxiety, and Studies suggest that BZ antagonists and inverse agonists (which induce the opposite effect to agonists at receptors) may be useful in alcohol rehabilitation.

Panel 2D Summary: Ag1680 Expression of the CG57654-01 gene is highest in a sample of normal colon tissues (CT=33.6). There appears to be a general pattern of overexpression of this gene in the normal matched colon samples when compared to the adjacent colon tumors. Therefore, therapeutic modulation of the activity of this gene or its protein product, using small molecule drugs, antibodies or protein therapeutics, may be of use in the treatment of colon cancer.

Panel 4D Summary: Ag1680 Expression of the CG57654-01 gene is highest in the lung-derived mucoepidermoid cell line (CT=34.7). Therefore, therapeutic modulation of the activity of this gene, using small molecule drugs or antibodies, may be of use in the treatment of asthma and emphysema.

NOV22

Expression of gene N0V22/CG57724-01 was assessed using the primer-probe set Ag3316, described in Table TA. Results of the RTQ-PCR runs are shown in Tables TB, TC and TD.

TABLE TA
Probe Name Ag3316

| Primers | Sequences | Length | Start Position | SEQ ID NO |
|---|---|---|---|---|
| Forward | 5′-tagttcaagatgccactttcgt-3′ | 22 | 1175 | 322 |
| Probe | TET-5′-actgcagactgctcactaccaccgag-3′-TAMRA | 26 | 1206 | 323 |
| Reverse | 5′-cgtggtgctcaaattcataca-3′ | 21 | 1253 | 324 |

TABLE TB
CNS_neurodegencration_v1.0

| | Rel. Exp. (%) | | Rel. Exp. (%) |
|---|---|---|---|
| | Ag3316, Run | | Ag3316, Run |
| Tissue Name | 210144080 | Tissue Name | 210144080 |
| AD 1 Hippo | 2.1 | Control (Path) 3 Temporal | 4.6 |
| | | Ctx | |
| AD 2 Hippo | 35.8 | Control (Path) 4 Temporal | 71.7 |
| | | Ctx | |
| AD 3 Hippo | 5.1 | AD 1 Occipital Ctx | 13.9 |
| AD 4 Hippo | 19.5 | AD 2 Occipital Ctx | 0.0 |
| | | (Missing) | |
| AD 5 Hippo | 26.1 | AD 3 Occipital Ctx | 1.4 |
| AD 6 Hippo | 81.8 | AD 4 Occipital Ctx | 37.9 |
| Control 2 Hippo | 52.9 | AD 5 Occipital Ctx | 55.5 |
| Control 4 Hippo | 5.2 | AD 6 Occipital Ctx | 35.1 |
| Control (Path) 3 Hippo | 4.2 | Control 1 Occipital Ctx | 2.8 |
| AD 1 Temporal Ctx | 18.4 | Control 2 Occipital Ctx | 40.9 |
| AD 2 Temporal Ctx | 47.6 | Control 3 Occipital Ctx | 31.9 |
| AD 3 Temporal Ctx | 2.5 | Control 4 Occipital Clx | 2.1 |
| AD 4 Temporal Ctx | 40.9 | Control (Path) 1 Occipital | 87.1 |
| | | Ctx | |
| AD 5 Inf Temporal Ctx | 74.2 | Control (Path) 2 Occipital | 17.2 |
| | | Ctx | |
| AD 5 Sup Temporal Ctx | 25.9 | Control (Path) 3 Occipital | 0.0 |
| | | Ctx | |
| AD 6 Inf Temporal Ctx | 100.0 | Control (Path) 4 Occipital | 29.7 |
| | | Ctx | |
| AD 6 Sup Temporal Ctx | 88.3 | Control 1 Parietal Ctx | 6.2 |
| Control 1 Temporal Ctx | 5.8 | Control 2 Parietal Ctx | 25.9 |
| Control 2 Temporal Ctx | 46.7 | Control 3 Parietal Ctx | 23.0 |
| Control 3 Temporal Ctx | 54.3 | Control (Path) 1 Parietal | 57.0 |
| | | Ctx | |
| Control 3 Temporal Ctx | 5.7 | Control (Path) 2 Parietal | 34.4 |
| | | Ctx | |
| Control (Path) 1 Temporal | 85.3 | Control (Path) 3 Parietal | 2.8 |
| Ctx | | Ctx | |
| Control (Path) 2 Temporal | 59.9 | Control (Path) 4 Parietal | 53.2 |
| Ctx | | Ctx | |

TABLE TC
General_screening_panel_v1.4

| | Rel. Exp. (%) | | Rel. Exp. (%) |
|---|---|---|---|
| | Ag3316, Run | | Ag3316, Run |
| Tissue Name | 215678598 | Tissue Name | 215678598 |
| Adipose | 12.4 | Renal ca. TK-10 | 0.4 |
| Melanoma* Hs688(A).T | 2.5 | Bladder | 8.1 |
| Melanoma* Hs688(B).T | 4.0 | Gastric ca. (liver met.) NCI- | 0.0 |
| | | N87 | |
| Melanoma* M14 | 0.0 | Gastric ca. KATO III | 0.6 |
| Melanoma* LOXIMVI | 0.0 | Colon ca. SW-948 | 2.7 |
| Melanoma* SK-MEL-5 | 0.0 | Colon ca. SW480 | 0.0 |
| Squamous cell carcinoma | 0.6 | Colon ca.* (SW480 met) | 0.0 |
| SCC-4 | | SW620 | |
| Testis Pool | 28.5 | Colon ca. HT29 | 3.0 |
| Prostate ca.* (bone met) | 8.8 | Colon ca. HCT-116 | 0.0 |
| PC-3 | | | |
| Prostate Pool | 11.3 | Colon ca. CaCo-2 | 0.0 |
| Placenta | 7.2 | Colon cancer tissue | 4.3 |
| Uterus Pool | 1.0 | Colon ca. SW1116 | 0.0 |
| Ovarian ca. OVCAR-3 | 8.5 | Colon ca. Colo-205 | 0.6 |
| Ovarian ca. SK-OV-3 | 4.6 | Colon ca. SW-48 | 3.5 |
| Ovarian ca. OVCAR-4 | 0.0 | Colon Pool | 3.7 |
| Ovarian ca. OVCAR-5 | 2.2 | Small Intestine Pool | 10.2 |
| Ovarian ca. IGROV-1 | 0.0 | Stomach Pool | 2.7 |
| Ovarian ca. OVCAR-8 | 1.0 | Bone Marrow Pool | 3.6 |
| Ovary | 2.9 | Fetal Heart | 1.5 |
| Breast ca. MCF-7 | 10.2 | Heart Pool | 2.6 |
| Breast ca. MDA-MB-231 | 6.5 | Lymph Node Pool | 3.8 |
| Breast ca. BT 549 | 12.1 | Fetal Skeletal Muscle | 1.7 |
| Breast ca. T47D | 2.7 | Skeletal Muscle Pool | 2.5 |
| Breast ca. MDA-N | 0.0 | Spleen Pool | 3.7 |
| Breast Pool | 4.5 | Thymus Pool | 8.4 |
| Trachea | 80.7 | CNS cancer (glio/astro) U87- | 0.2 |
| | | MG | |
| Lung | 2.1 | CNS cancer (glio/astro) U- | 0.4 |
| | | 118-MG | |
| Fetal Lung | 29.3 | CNS cancer (neuro;met) SK- | 1.1 |
| | | N-AS | |
| Lung ca. NCI-N417 | 0.0 | CNS cancer (astro) SF-539 | 0.0 |
| Lung ca. LX-1 | 2.0 | CNS cancer (astro) SNB-75 | 0.0 |
| Lung ca. NCI-H146 | 2.6 | CNS cancer (glio) SNB-19 | 0.4 |
| Lung ca. SHP-77 | 5.8 | CNS cancer (glio) SF-295 | 0.2 |
| Lung ca. A549 | 3.4 | Brain (Amygdala) Pool | 14.4 |
| Lung ca. NCI-H526 | 2.7 | Brain (cerebellum) | 35.6 |
| Lung ca. NCI-H23 | 100.0 | Brain (fetal) | 9.4 |
| Lung ca. NCI-H460 | 0.0 | Brain (Hippocampus) Pool | 11.8 |
| Lung ca. HOP-62 | 3.5 | Cerebral Cortex Pool | 18.0 |
| Lung ca. NCI-H522 | 14.2 | Brain (Substantia nigra) Pool | 12.0 |
| Liver | 13.4 | Brain (Thalamus) Pool | 17.4 |
| Fetal Liver | 12.3 | Brain (whole) | 17.6 |
| Liver ca. HepG2 | 0.0 | Spinal Cord Pool | 5.9 |
| Kidney Pool | 6.0 | Adrenal Gland | 22.5 |
| Fetal Kidney | 16.2 | Pituitary gland Pool | 31.0 |
| Renal ca. 786-0 | 0.8 | Salivary Gland | 32.3 |
| Renal ca. A498 | 4.9 | Thyroid (female) | 7.3 |
| Renal ca. ACHN | 4.2 | Pancreatic ca. CAPAN2 | 10.2 |
| Renal ca. UO-31 | 2.3 | Pancreas Pool | 8.0 |

TABLE TD
Panel 4D

| | Rel. Exp. (%) | | Rel. Exp. (%) |
|---|---|---|---|
| | Ag3316, Run | | Ag3316, Run |
| Tissue Name | 164683048 | Tissue Name | 164683048 |
| Secondary Th1 act | 46.0 | HUVEC IL-1beta | 7.8 |
| Secondary Th2 act | 60.7 | HUVEC IFN gamma | 5.0 |
| Secondary Tr1 act | 88.3 | HUVEC TNF alpha + IFN gamma | 2.2 |
| Secondary Th1 rest | 31.2 | HUVEC TNF alpha + IL4 | 2.5 |
| Secondary Th2 rest | 57.4 | HUVEC IL-11 | 2.4 |
| Secondary Tr1 rest | 72.7 | Lung Microvascular EC none | 7.5 |
| Primary Th1 act | 23.3 | Lung Microvascular EC TNFalpha | 3.8 |
| | | + IL-1beta | |
| Primary Th2 act | 59.5 | Microvascular Dermal EC none | 7.8 |
| Primary Tr1 act | 76.8 | Microsvasular Dermal EC | 2.7 |
| | | TNFalpha + IL-1beta | |
| Primary Th1 rest | 90.1 | Bronchial epithelium TNFalpha + | 8.8 |
| | | IL1beta | |
| Primary Th2 rest | 87.7 | Small airway epithelium none | 2.7 |
| Primary Tr1 rest | 99.3 | Small airway epithelium TNFalpha | 37.9 |
| | | + IL-1beta | |
| CD45RA CD4 lymphocyte | 5.3 | Coronery artery SMC rest | 2.2 |
| act | | | |
| CD45RO CD4 lymphocyte | 35.8 | Coronery artery SMC TNFalpha + | 4.5 |
| act | | IL-1beta | |
| CD8 lymphocyte act | 16.5 | Astrocytes rest | 9.8 |
| Secondary CD8 lymphocyte | 38.2 | Astrocytes TNFalpha + IL-1beta | 2.2 |
| rest | | | |
| Secondary CD8 lymphocyte | 14.7 | KU-812 (Basophil) rest | 0.0 |
| act | | | |
| CD4 lymphocyte none | 18.9 | KU-812 (Basophil) | 1.7 |
| | | PMA/ionomycin | |
| 2ry Th1/Th2/Tr1_anti-CD95 | 100.0 | CCD1106 (Keratinocytes) none | 34.6 |
| CH11 | | | |
| LAK cells rest | 64.6 | CCD1106 (Keratinocytes) | 32.3 |
| | | TNFalpha + IL-lbeta | |
| LAK cells IL-2 | 2.3 | Liver cirrhosis | 11.4 |
| LAK cells IL-2 + IL-12 | 19.3 | Lupus kidney | 10.7 |
| LAK cells IL-2 + IFN gamma | 60.7 | NCI-H292 none | 25.0 |
| LAK cells IL-2 + IL-18 | 34.4 | NCI-H292 IL-4 | 20.7 |
| LAK cells PMA/ionomycin | 37.6 | NCI-H292 IL-9 | 25.5 |
| NK Cells IL-2 rest | 6.1 | NCI-H292 IL-I3 | 7.6 |
| Two Way MLR 3 day | 8.9 | NCI-H292 IFN gamma | 7.0 |
| Two Way MLR 5 day | 2.0 | HPAEC none | 0.0 |
| Two Way MLR 7 day | 1.4 | HPAEC TNF alpha + IL-1 beta | 0.0 |
| PBMC rest | 4.7 | Lung fibroblast none | 3.4 |
| PBMC PWM | 48.6 | Lung fibroblast TNF alpha + IL-1 | 5.1 |
| | | beta | |
| PBMC PHA-L | 33.4 | Lung fibroblast IL-4 | 12.6 |
| Ramos (B cell) none | 0.0 | Lung fibroblast IL-9 | 5.8 |
| Ramos (B cell) ionomycin | 0.0 | Lung fibroblast IL-13 | 4.8 |
| B lymphocytes PWM | 68.8 | Lung fibroblast IFN gamma | 2.3 |
| B lymphocytes CD40L and | 33.2 | Dermal fibroblast CCD1070 rest | 20.7 |
| IL-4 | | | |
| EOL-1 dbcAMP | 0.0 | Dermal fibroblast CCD1070 TNF | 64.6 |
| | | alpha | |
| EOL-1 dbcAMP | 0.0 | Dermal fibroblast CCD1070 IL-1 | 2.2 |
| PMA/ionomycin | | beta | |
| Dendritic cells none | 9.8 | Dermal fibroblast IFN gamma | 6.1 |
| Dendritic cells LPS | 6.3 | Dermal fibroblast IL-4 | 8.2 |
| Dendritic cells anti-CD40 | 14.5 | IBD Colitis 2 | 0.0 |
| Monocytes rest | 41.2 | IBD Crohn's | 4.5 |
| Monocytes LPS | 0.0 | Colon | 16.7 |
| Macrophages rest | 4.6 | Lung | 8.5 |
| Macrophages LPS | 4.8 | Thymus | 93.3 |
| HUVEC none | 0.0 | Kidney | 17.1 |
| HUVEC starved | 12.4 | | |

CNS_neurodegeneration_v1.0 Summary: Ag3316 This panel confirms the expression of this gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3316 Expression of the CG57724-01 gene is highest in lung cancer cell line NCI-H23 (CT=29.4). This gene is also expressed at moderate levels in all regions of the central nervous system examined, including in amygdala, cerebellum, hippocampus, cerebral cortex, substantia nigra, thalamus, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at moderate levels in adrenal gland and pituitary gland and at low levels in pancreas, thyroid, liver, and adipose. Therefore, therapeutic modulation of the activity of this gene may prove useful in the treatment of endocrine/metabolically related diseases, such as obesity and diabetes.

The CG57724-01 gene encodes a protein with homology to carboxylesterases. Carboxylesterases (CESs) catalyze the hydrolysis of a variety of compounds containing ester and amide bonds. They may also play an important role in lipid and drug metabolism by hydrolyzing endogenous long-chain fatty acid esters.

Panel 4D Summary: Ag3316 Moderate to low expression of this gene is found in a wide range of cells included in this panel, with a highest expression in activated T cells and in particular T regulatory cells (Tr1). This gene encodes for a carboxylesterase like protein, a protein that plays an important role in the metabolism of endogenous lipids. Therefore regulation of carboxylesterase gene expression may have physiological significance essential in T cell mediated diseases such as autoimmune diseases and allergies as well as in B cell disorders.

NOV25

Expression of gene NOV25/CG57503-01 was assessed using the primer-probe set Ag3258, described in Table UA. Results of the RTQ-PCR runs are shown in Tables UB, UC and UD.

TABLE UA
Probe Name Ag3258

| Primers | Sequences | Length | Start Position | SEQ ID NO |
|---|---|---|---|---|
| Forward | 5′-gagtcaccgtaaggctgtca-3′ | 20 | 1728 | 325 |
| Probe | TET-5′-catcccttcgccatcacagtgtttg-3′-TAMRA | 25 | 1765 | 326 |
| Reverse | 5′-tctgtccagtacaggctgtctt-3′ | 22 | 1790 | 327 |

TABLE UB
CNS_neurodegeneration_v1.0

| | Rel. Exp. (%) | | Rel. Exp. (%) |
|---|---|---|---|
| | Ag3258, Run | | Ag3258, Run |
| Tissue Name | 209990877 | Tissue Name | 209990877 |
| AD 1 Hippo | 56.6 | Control (Path) 3 Temporal | 42.3 |
| | | Ctx | |
| AD 2 Hippo | 44.8 | Control (Path) 4 Temporal | 49.3 |
| | | Ctx | |
| AD 3 Hippo | 42.6 | AD 1 Occipital Ctx | 56.6 |
| AD 4 Hippo | 23.3 | AD 2 Occipital Ctx | 0.0 |
| | | (Missing) | |
| AD 5 Hippo | 96.6 | AD 3 Occipital Ctx | 68.8 |
| AD 6 Hippo | 77.4 | AD 4 Occipital Ctx | 50.3 |
| Control 2 Hippo | 39.0 | AD 5 Occipital Ctx | 43.8 |
| Control 4 Hippo | 39.5 | AD 6 Occipital Ctx | 38.4 |
| Control (Path) 3 Hippo | 30.1 | Control 1 Occipital Ctx | 27.4 |
| AD 1 Temporal Ctx | 90.8 | Control 2 Occipital Ctx | 34.2 |
| AD 2 Temporal Ctx | 55.5 | Control 3 Occipital Ctx | 39.0 |
| AD 3 Temporal Ctx | 46.0 | Control 4 Occipital Ctx | 27.7 |
| AD 4 Temporal Ctx | 47.3 | Control (Path) 1 Occipital | 53.6 |
| | | Ctx | |
| AD 5 Inf Temporal Ctx | 100.0 | Control (Path) 2 Occipital | 23.0 |
| | | Ctx | |
| AD 5 Sup Temporal Ctx | 88.9 | Control (Path) 3 Occipital | 24.3 |
| | | Ctx | |
| AD 6 Inf Temporal Ctx | 79.6 | Control (Path) 4 Occipital | 19.1 |
| | | Ctx | |
| AD 6 Sup Temporal Ctx | 57.0 | Control 1 Parietal Ctx | 33.2 |
| Control 1 Temporal Ctx | 36.1 | Control 2 Parietal Ctx | 85.9 |
| Control 2 Temporal Ctx | 35.8 | Control 3 Parietal Ctx | 25.9 |
| Control 3 Temporal Ctx | 42.6 | Control (Path) 1 Parietal | 68.8 |
| | | Ctx | |
| Control 3 Temporal Ctx | 26.8 | Control (Path) 2 Parietal | 47.6 |
| | | Ctx | |
| Control (Path) 1 Temporal | 53.2 | Control (Path) 3 Parietal | 26.2 |
| Ctx | | Ctx | |
| Control (Path) 2 Temporal | 39.8 | Control (Path) 4 Parietal | 39.8 |
| Ctx | | Ctx | |

TABLE UC
General_screening_panel_v1.4

| | Rel. Exp. (%) | | Rel. Exp. (%) |
|---|---|---|---|
| | Ag3258, Run | | Ag3258, Run |
| Tissue Name | 214694854 | Tissue Name | 214694854 |
| Adipose | 4.6 | Renal ca. TK-10 | 13.1 |
| Melanoma* Hs688(A).T | 13.3 | Bladder | 3.1 |
| Melanoma* Hs688(B).T | 7.3 | Gastric ca. (liver met.) NCI- | 1.5 |
| | | N87 | |
| Melanoma* M14 | 22.4 | Gastric ca. KATO III | 0.0 |
| Melanoma* LOXIMVI | 26.1 | Colon ca. SW-948 | 6.7 |
| Melanoma* SK-MEL-5 | 50.7 | Colon ca. SW480 | 7.7 |
| Squamous cell carcinoma | 11.8 | Colon ca.* (SW480 met) | 17.8 |
| SCC-4 | | SW620 | |
| Testis Pool | 3.8 | Colon ca. HT29 | 0.0 |
| Prostate ca.* (bone met) | 3.6 | Colon ca. HCT-116 | 12.9 |
| PC-3 | | | |
| Prostate Pool | 7.3 | Colon ca. CaCo-2 | 19.8 |
| Placenta | 7.7 | Colon cancer tissue | 4.4 |
| Uterus Pool | 3.1 | Colon ca. SW1116 | 0.9 |
| Ovarian ca. OVCAR-3 | 4.5 | Colon ca. Colo-205 | 56.3 |
| Ovarian ca. SK-OV-3 | 10.7 | Colon ca. SW-48 | 100.0 |
| Ovarian ca. OVCAR-4 | 0.5 | Colon Pool | 5.4 |
| Ovarian ca. OVCAR-5 | 7.2 | Small Intestine Pool | 14.4 |
| Ovarian ca. IGROV-1 | 12.5 | Stomach Pool | 6.0 |
| Ovarian ca. OVCAR-8 | 6.5 | Bone Marrow Pool | 4.1 |
| Ovary | 18.8 | Fetal Heart | 12.7 |
| Breast ca. MCF-7 | 0.2 | Heart Pool | 14.3 |
| Breast ca. MDA-MB-231 | 5.6 | Lymph Node Pool | 7.4 |
| Breast ca. BT 549 | 1.0 | Fetal Skeletal Muscle | 14.8 |
| Breast ca. T47D | 9.0 | Skeletal Muscle Pool | 43.5 |
| Breast ca. MDA-N | 0.6 | Spleen Pool | 16.7 |
| Breast Pool | 5.6 | Thymus Pool | 8.5 |
| Trachea | 12.4 | CNS cancer (glio/astro) U87- | 7.5 |
| | | MG | |
| Lung | 2.1 | CNS cancer (glio/astro) U- | 58.6 |
| | | 118-MG | |
| Fetal Lung | 83.5 | CNS cancer (neuro; met) SK- | 23.7 |
| | | N-AS | |
| Lung ca. NCI-N417 | 3.4 | CNS cancer (astro) SF-539 | 1.9 |
| Lung ca. LX-1 | 2.6 | CNS cancer (astro) SNB-75 | 1.5 |
| Lung ca. NCI-H146 | 20.9 | CNS cancer (glio) SNB-19 | 12.2 |
| Lung ca. SHP-77 | 20.9 | CNS cancer (glio) SF-295 | 4.8 |
| Lung ca. A549 | 37.9 | Brain (Amygdala) Pool | 28.9 |
| Lung ca. NCI-H526 | 10.2 | Brain (cerebellum) | 59.9 |
| Lung ca. NCI-H23 | 16.2 | Brain (fetal) | 34.2 |
| Lung ca. NCI-H460 | 5.2 | Brain (Hippocampus) Pool | 40.6 |
| Lung ca. HOP-62 | 3.1 | Cerebral Cortex Pool | 62.9 |
| Lung ca. NCI-H522 | 38.2 | Brain (Substantia nigra) Pool | 39.0 |
| Liver | 0.9 | Brain (Thalamus) Pool | 56.6 |
| Fetal Liver | 2.2 | Brain (whole) | 58.2 |
| Liver ca. HepG2 | 14.0 | Spinal Cord Pool | 58.2 |
| Kidney Pool | 15.9 | Adrenal Gland | 12.1 |
| Fetal Kidney | 21.6 | Pituitary gland Pool | 6.7 |
| Renal ca. 786-0 | 6.6 | Salivary Gland | 4.8 |
| Renal ca. A498 | 3.7 | Thyroid (female) | 13.1 |
| Renal ca. ACHN | 19.2 | Pancreatic ca. CAPAN2 | 0.1 |
| Renal ca. UO-31 | 8.2 | Pancreas Pool | 10.5 |

TABLE UD
Panel 4D

| | Rel. Exp. (%) | | Rel. Exp. (%) |
|---|---|---|---|
| | Ag3258, Run | | Ag3258, Run |
| Tissue Name | 164537289 | Tissue Name | 164537289 |
| Secondary Th1 act | 0.3 | HUVEC IL-1beta | 3.2 |
| Secondary Th2 act | 3.8 | HUVEC IFN gamma | 6.5 |
| Secondary Tr1 act | 0.3 | HUVEC TNF alpha + IFN gamma | 4.2 |
| Secondary Th1 rest | 0.2 | HUVEC TNF alpha + IL4 | 5.2 |
| Secondary Th2 rest | 0.0 | HUVEC IL-11 | 3.4 |
| Secondary Tr1 rest | 0.0 | Lung Microvascular EC none | 4.4 |
| Primary Th1 act | 2.4 | Lung Microvascular EC | 1.8 |
| | | TNFalpha + IL-1beta | |
| Primary Th2 act | 0.0 | Microvascular Dermal EC none | 8.5 |
| Primary Tr1 act | 0.0 | Microsvasular Dermal EC | 4.9 |
| | | TNFalpha + IL-1 beta | |
| Primary Th1 rest | 0.0 | Bronchial epithelium TNFalpha + | 14.9 |
| | | IL1beta | |
| Primary Th2 rest | 0.2 | Small airway epithelium none | 2.1 |
| Primary Tr1 rest | 0.0 | Small airway epithelium | 19.3 |
| | | TNFalpha + IL-1beta | |
| CD45RA CD4 lymphocyte | 3.0 | Coronery artery SMC rest | 13.3 |
| act | | | |
| CD45RO CD4 lymphocyte | 0.0 | Coronery artery SMC TNFalpha + | 9.4 |
| act | | IL-1beta | |
| CD8 lymphocyte act | 0.0 | Astrocytes rest | 21.9 |
| Secondary CD8 lymphocyte | 0.0 | Astrocytes TNFalpha + IL-1 beta | 3.5 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 0.3 |
| | | PMA/ionomycin | |
| 2ry Th1/Th2/Tr1_anti-CD95 | 0.0 | CCD1106 (Keratinocytes) none | 24.5 |
| CH11 | | | |
| LAK cells rest | 1.0 | CCD1106 (Keratinocytes) | 6.2 |
| | | TNFalpha + IL-1beta | |
| LAK cells IL-2 | 0.4 | Liver cirrhosis | 1.0 |
| LAK cells IL-2 + IL-12 | 0.0 | Lupus kidney | 3.9 |
| LAK cells IL-2 + IFN gamma | 3.1 | NCI-H292 none | 78.5 |
| LAK cells IL-2 + IL-18 | 0.7 | NCI-H292 IL-4 | 100.0 |
| LAK cells PMA/ionomycin | 0.0 | NCI-H292 IL-9 | 87.1 |
| NK Cells IL-2 rest | 0.0 | NCI-H292 IL-13 | 53.2 |
| Two Way MLR 3 day | 0.0 | NCI-H292 IFN gamma | 37.6 |
| Two Way MLR 5 day | 0.0 | HPAEC none | 1.3 |
| Two Way MLR 7 day | 0.0 | HPAEC TNF alpha + IL-1 beta | 2.9 |
| PBMC rest | 0.0 | Lung fibroblast none | 16.5 |
| PBMC PWM | 2.1 | Lung fibroblast TNF alpha + IL-1 | 14.0 |
| | | beta | |
| PBMC PHA-L | 0.3 | Lung fibroblast IL-4 | 21.8 |
| Ramos (B cell) none | 13.1 | Lung fibroblast IL-9 | 17.4 |
| Ramos (B cell) ionomycin | 42.3 | Lung fibroblast IL-13 | 13.6 |
| B lymphocytes PWM | 4.2 | Lung fibroblast IFN gamma | 20.6 |
| B lymphocytes CD40L and | 0.4 | Dermal fibroblast CCD1070 rest | 20.6 |
| IL-4 | | | |
| EOL-1 dbcAMP | 0.0 | Dermal fibroblast CCD1070 TNF | 12.9 |
| | | alpha | |
| EOL-1 dbcAMP | 0.0 | Dermal fibroblast CCD1070 IL-1 | 20.3 |
| PMA/ionomycin | | beta | |
| Dendritic cells none | 0.0 | Dermal fibroblast IFN gamma | 13.3 |
| Dendritic cells LPS | 0.0 | Dermal fibroblast IL-4 | 33.7 |
| Dendritic cells anti-CD40 | 0.0 | IBD Colitis 2 | 1.6 |
| Monocytes rest | 0.0 | IBD Crohn's | 9.8 |
| Monocytes LPS | 0.0 | Colon | 52.9 |
| Macrophages rest | 0.0 | Lung | 17.3 |
| Macrophages LPS | 0.3 | Thymus | 13.4 |
| HUVEC none | 5.9 | Kidney | 29.3 |
| HUVEC starved | 10.3 | | |

CNS_neurodegeneration_v1.0 Summary: Ag3258 This panel confirms the expression of this gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3258 This gene is expressed at high to moderate levels in the majority of samples on this panel. In particular, high levels of CG57503-01 gene expression are seen in all regions of the central nervous system examined, including in amygdala, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

The expression of this gene in metabolic tissues such as pancreas, thyroid, pituitary gland, adrenal gland, skeletal muscle, heart, adipose, and liver suggests that modulation of the activity of this gene may prove useful in the treatment of endocrine/metabolically related diseases, such as obesity and diabetes.

In addition, there is substantial expression of this gene associated with several cancer cell lines, particularly melanoma and lung cancer cell lines. Therefore, therapeutic modulation of the activity of this gene or its protein product, through the use of small molecule drugs, protein therapeutics or antibodies, might be beneficial in the treatment of melanoma and lung cancer.

Interestingly, this gene is expressed at much higher levels in fetal lung (CT=25.6) than in adult lung (CT=31.0). This observation suggests that expression of this gene can be used to distinguish fetal from adult lung tissue.

Panel 4D Summary: Ag3258 Expression of the CG57503-01 gene is highest in the lung-derived mucoepidermoid cell line NCI-H1292 (CT=28.1). Thus, therapeutic modulation of the activity of this gene, using small molecule drugs, antibodies or protein therapeutics, may be of use in the treatment of asthma and emphysema. Low to moderate expression of this gene is also seen in a number of fibroblast and epithelial cell lines.

The CG57503-01 gene encodes a protein with homology to MEGF7 and is a Type I membrane protein with 7 LDL-R type A domains, 19 LDL-R type B domains, 11 EGF domains, and a cytoplasmic tail of >100 residues. This protein may act as a cell surface receptor for an uncharacterized ligand and play a role in Ca++-dependent functions, as is the case for other proteins with multiple EGF domains. Expression of the CG57503-01 gene at moderate levels in the brain, in lung and skin fibroblasts, and selectively in the pulmonary epidermoid carcinoma cell line NCI-H292, suggests that therapeutic antibodies and small molecule antagonists that block its function may be useful in reduction or elimination of the symptoms of asthma, emphysema, or psoriasis.

NOV27

Expression of gene NOV27/CG57658-01 was assessed using the primer-probe set Ag3299, described in Table VA. Results of the RTQ-PCR runs are shown in Tables VB, VC and VD.

TABLE VA
Probe Name Ag3299

| Primers | Sequences | Length | Start Position | SEQ ID NO |
|---|---|---|---|---|
| Forward | 5′-cttcctctcttccaggaaagc-3′ | 21 | 14 | 328 |
| Probe | TET-5′-tctggttcgtcctcacgatgctg-3′-TAMRA | 23 | 35 | 329 |
| Reverse | 5′-tcctgctcgtcctggtaga-3′ | 19 | 95 | 340 |

TABLE VB
CNS_neurodegeneration_v1.0

| | Rel. Exp.(%) Ag3299, |
|---|---|
| Tissue Name | Run 210063511 |
| AD 1 Hippo | 0.0 |
| AD 2 Hippo | 12.0 |
| AD 3 Hippo | 0.0 |
| AD 4 Hippo | 3.3 |
| AD 5 hippo | 78.5 |
| AD 6 Hippo | 13.9 |
| Control 2 Hippo | 22.8 |
| Control 4 Hippo | 0.0 |
| Control (Path) 3 Hippo | 5.4 |
| AD 1 Temporal Ctx | 10.2 |
| AD 2 Temporal Ctx | 51.8 |
| AD 3 Temporal Ctx | 12.9 |
| AD 4 Temporal Ctx | 22.1 |
| AD 5 Inf Temporal Ctx | 72.7 |
| AD 5 Sup Temporal Ctx | 6.0 |
| AD 6 Inf Temporal Ctx | 32.3 |
| AD 6 Sup Temporal Ctx | 32.8 |
| Control 1 Temporal Ctx | 0.0 |
| Control 2 Temporal Ctx | 37.4 |
| Control 3 Temporal Ctx | 20.4 |
| Control 4 Temporal Ctx | 3.2 |
| Control (Path) 1 Temporal Ctx | 94.0 |
| Control (Path) 2 Temporal Ctx | 45.7 |
| Control (Path) 3 Temporal Ctx | 6.3 |
| Control (Path) 4 Temporal Ctx | 79.6 |
| AD 1 Occipital Ctx | 21.5 |
| AD 2 Occipital Ctx | 0.0 |
| (Missing) | |
| AD 3 Occipital Ctx | 4.1 |
| AD 4 Occipital Ctx | 23.3 |
| AD 5 Occipital Ctx | 8.0 |
| AD 6 Occipital Ctx | 44.4 |
| Control 1 Occipital Ctx | 0.0 |
| Control 2 Occipital Ctx | 89.5 |
| Control 3 Occipital Ctx | 16.8 |
| Control 4 Occipital Ctx | 0.0 |
| Control (Path) 1 Occipital Ctx | 66.4 |
| Control (Path) 2 Occipital Ctx | 20.4 |
| Control (Path) 3 Occipital Ctx | 0.0 |
| Control (Path) 4 Occipital Ctx | 21.5 |
| Control 1 Parietal Ctx | 10.2 |
| Control 2 Parietal Ctx | 35.1 |
| Control 3 Parietal Ctx | 21.5 |
| Control (Path) 1 Parietal Ctx | 100.0 |
| Control (Path) 2 Parietal Ctx | 22.2 |
| Control (Path) 3 Parietal Ctx | 0.0 |
| Control (Path) 4 Parietal Ctx | 66.9 |

TABLE VC
General_screening_panel_v1.4

| | Rel. Exp.(%) Ag3299, |
|---|---|
| Tissue Name | Run 215620706 |
| Adipose | 2.1 |
| Melanoma* Hs688(A).T | 4.4 |
| Melanoma* Hs688(B).T | 12.1 |
| Melanoma* M14 | 0.0 |
| Melanoma* LOXIMVI | 4.1 |
| Melanoma* SK-MEL-5 | 0.0 |
| Squamous cell carcinoma | 0.0 |
| SCC-4 | |
| Testis Pool | 8.5 |
| Prostate ca.* (bone met) | 100.0 |
| PC-3 | |
| Prostate Pool | 6.7 |
| Placenta | 0.0 |
| Uterus Pool | 0.0 |
| Ovarian ca. OVCAR-3 | 6.7 |
| Ovarian ca. SK-OV-3 | 6.0 |
| Ovarian ca. OVCAR-4 | 0.0 |
| Ovarian ca. OVCAR-5 | 2.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 8.4 |
| Breast ca. BT 549 | 6.8 |
| Breast ca. T47D | 2.5 |
| Breast ca. MDA-N | 0.0 |
| Breast Pool | 6.1 |
| Trachea | 0.0 |
| Lung | 1.8 |
| Fetal Lung | 6.8 |
| Lung ca. NCI-N417 | 0.0 |
| Lung ca. LX-1 | 0.0 |
| Lung ca. NCI-H146 | 13.8 |
| Lung ca. SHP-77 | 3.9 |
| Lung ca. A549 | 0.0 |
| Lung ca. NCI-H526 | 0.0 |
| Lung ca. NCI-H23 | 47.0 |
| Lung ca. NCI-H460 | 10.2 |
| Lung ca. HOP-62 | 2.2 |
| Lung ca. NCI-H522 | 0.0 |
| Liver | 0.0 |
| Fetal Liver | 0.0 |
| Liver ca. HepG2 | 0.0 |
| Kidney Pool | 9.8 |
| Fetal Kidney | 17.6 |
| Renal ca. 786-0 | 2.6 |
| Renal ca. A498 | 0.0 |
| Renal ca. ACHN | 0.0 |
| Renal ca. UO-31 | 0.0 |
| Renal ca. TK-10 | 2.8 |
| Bladder | 0.0 |
| Gastric ca (liver met.) NCI-N87 | 7.3 |
| Gastric ca. KATO III | 1.1 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 4.4 |
| Colon ca. CaCo-2 | 0.0 |
| Colon cancer tissue | 4.0 |
| Colon ca. SW1116 | 0.0 |
| Colon ca. Colo-205 | 0.0 |
| Colon ca. SW-48 | 0.0 |
| Colon Pool | 7.6 |
| Small Intestine Pool | 9.7 |
| Stomach Pool | 17.0 |
| Bone Marrow Pool | 2.8 |
| Fetal Heart | 2.1 |
| Heart Pool | 0.0 |
| Lymph Node Pool | 7.0 |
| Fetal Skeletal Muscle | 2.2 |
| Skeletal Muscle Pool | 0.0 |
| Spleen Pool | 4.1 |
| Thymus Pool | 12.9 |
| CNS cancer (glio/astro) U87- | 2.1 |
| MG | |
| CNS cancer (glio/astro) U- | 3.6 |
| 118-MG | |
| CNS cancer (neuro;met) SK- | 0.0 |
| N-AS | |
| CNS cancer (astro) SF-539 | 0.0 |
| CNS cancer (astro) SNB-75 | 2.0 |
| CNS cancer (glio) SNB-19 | 1.3 |
| CNS cancer (glio) SF-295 | 0.0 |
| Brain (Amygdala) Pool | 21.2 |
| Brain (cerebellum) | 5.5 |
| Brain (fetal) | 14.3 |
| Brain (Hippocampus) Pool | 13.9 |
| Cerebral Cortex Pool | 38.7 |
| Brain (Substantia nigra) Pool | 20.9 |
| Brain (Thalamus) Pool | 33.4 |
| Brain (whole) | 36.1 |
| Spinal Cord Pool | 4.2 |
| Adrenal Gland | 3.8 |
| Pituitary gland Pool | 4.1 |
| Salivary Gland | 0.0 |
| Thyroid (female) | 0.0 |
| Pancreatic ca. CAPAN2 | 3.0 |
| Pancreas Pool | 2.1 |

TABLE VD
Panel 4D

| | Rel. Exp. (%) | | Rel. Exp. (%) |
|---|---|---|---|
| | Ag3299, Run | | Ag3299, Run |
| Tissue Name | 164682522 | Tissue Name | 164682522 |
| Secondary Th1 act | 2.4 | HUVEC IL-1beta | 0.0 |
| Secondary Th2 act | 0.0 | HUVEC IFN gamma | 0.0 |
| Secondary Tr1 act | 7.2 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 11.2 |
| Secondary Tr1 rest | 0.0 | Lung Microvascular EC none | 25.5 |
| Primary Th1 act | 0.0 | Lung Microvascular EC | 18.9 |
| | | TNFalpha + IL-1beta | |
| Primary Th2 act | 0.0 | Microvascular Dermal EC none | 9.9 |
| Primary Tr1 act | 8.5 | Microsvasular Dermal EC | 0.0 |
| | | TNFalpha + IL-1beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium | 12.5 |
| | | TNFalpha + IL-1beta | |
| CD45RA CD4 lymphocyte | 0.0 | Coronery artery SMC rest | 19.8 |
| act | | | |
| CD45RO CD4 lymphocyte | 0.0 | Coronery artery SMC TNFalpha + | 9.0 |
| act | | IL-1beta | |
| CD8 lymphocyte act | 0.0 | Astrocytes rest | 0.0 |
| Secondary CD8 lymphocyte | 0.0 | Astrocytes TNFalpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNFalpha + IL-1beta | |
| LAK cells IL-2 | 0.0 | Liver cirrhosis | 66.4 |
| LAK cells IL-2 + IL-12 | 0.0 | Lupus kidney | 0.0 |
| LAK cells IL-2 + IFN gamma | 0.0 | NCI-H292 none | 10.2 |
| LAK cells IL-2 + IL-18 | 13.3 | NCI-H292 IL-4 | 0.0 |
| LAK cells PMA/ionomycin | 0.0 | NCI-H292 IL-9 | 9.7 |
| NK Cells IL-2 rest | 12.5 | NCI-H292 IL-13 | 19.6 |
| Two Way MLR 3 day | 0.0 | NCI-H292 IFN gamma | 0.0 |
| Two Way MLR 5 day | 8.6 | HPAEC none | 9.2 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 11.3 | Lung fibroblast TNF alpha + IL-1 | 0.0 |
| | | beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 9.7 |
| B lymphocytes PWM | 23.5 | Lung fibroblast IFN gamma | 0.0 |
| B lymphocytes CD40L and | 0.0 | Dermal fibroblast CCD1070 rest | 19.5 |
| IL-4 | | | |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL-1 | 7.7 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 11.2 | IBD Crohn's | 0.0 |
| Monocytes LPS | 0.0 | Colon | 100.0 |
| Macrophages rest | 0.0 | Lung | 41.2 |
| Macrophages LPS | 0.0 | Thymus | 0.0 |
| HUVEC none | 5.9 | Kidney | 9.2 |
| HUVEC starved | 0.0 | | |

CNS_neurodegeneration_v1.0 Summary: Ag3299 This panel confirms the expression of this gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3299 The CG57658-01 gene is expressed at highest levels in prostate cancer cell line PC-3 (CT=30.5). Interestingly, this gene is expressed at much lower levels in the normal prostate. Therefore, therapeutic modulation of the activity of this gene, using small molecule drugs, antibodies or protein therapeutics, may be of benefit in the treatment of prostate cancer.

This gene is also expressed at low levels in all regions of the brain examined, including amygdala, cerebral cortex, cerebellum, substantia nigra, thalamus and hippocampus. The CG57658-01 gene encodes a protein that is a variant of the human CONNEXIN40.1 gene. Cell-specific expression of connexins in the central nervous system has been shown to regulate gap junctional coupling between glial cells and neurons (ref. 1). Therefore, modulation of the activity of this gene or its protein product may be useful in the treatment of inherited demyelinating neuropathies or other neurological diseases.

References:

1. Rash J E, Yasumura T, Dudek F E, Nagy J I. Cell-specific expression of connexins and evidence of restricted gap junctional coupling between glial cells and between neurons. J Neurosci Mar. 15, 2001;21(6):1983-2000

The transmembrane connexin proteins of gap junctions link extracellularly to form channels for cell-to-cell exchange of ions and small molecules. Two primary hypotheses of gap junction coupling in the CNS are the following: (1) generalized coupling occurs between neurons and glia, with some connexins expressed in both neurons and glia, and (2) intercellular junctional coupling is restricted to specific coupling partners, with different connexins expressed in each cell type. There is consensus that gap junctions link neurons to neurons and astrocytes to oligodendrocytes, ependymocytes, and other astrocytes. However, unresolved are the existence and degree to which gap junctions occur between oligodendrocytes, between oligodendrocytes and neurons, and between astrocytes and neurons. Using light microscopic immunocytochemistry and freeze-fracture replica immunogold labeling of adult rat CNS, we investigated whether four of the best-characterized CNS connexins are each present in one or more cell types, whether oligodendrocytes also share gap junctions with other oligodendrocytes or with neurons, and whether astrocytes share gap junctions with neurons. Connexin32 (Cx32) was found only in gap junctions of oligodendrocyte plasma membranes, Cx30 and Cx43 were found only in astrocyte membranes, and Cx36 was only in neurons. Oligodendrocytes shared intercellular gap junctions only with astrocytes, with each oligodendrocyte isolated from other oligodendrocytes except via astrocyte intermediaries. Finally, neurons shared gap junctions only with other neurons and not with glial cells. Thus, the different cell ypes of the CNS express different connexins, which define separate pathways for neuronal versus glial gap junctional communication.

PMID: 11245683

Panel 4D Summary: Ag3299 The CG57658-01 gene, which encodes a variant of the human CONNEXIN40.1 gene, is expressed at highest levels in the colon (CT=30) and appears to be down-regulated in colon tissue isolated from Crohn's and colitis patients (CT=40). Connexin is expressed in GAP junctions in gastrointestinal muscle (ref. 1). Thus, the expression of the transcript or the protein it encodes could be used to detect normal colon tissue. Furthermore, therapeutic modulation of the activity of this gene or its protein product could be useful in the treatment of inflammatory bowel disease.

References:

1. Wang Y F, Daniel E E. Gap junctions in gastrointestinal muscle contain multiple connexins. Am J Physiol Gastrointest Liver Physiol 2001 August;281(2):G533-43.

In the canine gastrointestinal tract, the roles that gap junctions play in pacemaking and neurotransmission are unclear. Using antibodies to connexin (Cx)₄₃, Cx45, and Cx40, we determined the distribution of these connexins. Cx43 was present in all locations where structural gap junctions occur. Cx40 was also widely distributed in the circular muscle of the lower esophageal sphincter (LES), stomach, and ileum. Cx45 was sparsely distributed in circular muscle of the LES. In the interstitial cells of Cajal (ICC) networks of myenteric plexus, in the deep muscular and submuscular plexuses, sparse Cx45 and Cx40 immunoreactivity was present. In colon, immunoreactivity was found only in the myenteric and submuscular plexus and nearby circular muscle cells. No immunoreactivity was found in sites lacking structural gap junctions (longitudinal muscle, inner circular muscle of the intestine, and most circular muscle of the colon). Studies of colocalization of connexilns suggested that in the ICC networks, some colocalization of Cx43 with Cx40 and/or Cx45 occurred. Thus gap junctions in canine intestine may be heterotypic or heteromeric and have different conductance properties in different regions based on different connexin compositions.

PMID: 11447034

NOV28

Expression of gene NOV28/CG57662-01 was assessed using the primer-probe set Ag3300, described in Table WA. Results of the RTQ-PCR runs are shown in Tables WB, WC and WD.

TABLE WA
Probe Name Ag3300

| Primers | Sequences | Length | Start Position | SEQ ID NO |
|---|---|---|---|---|
| Forward | 5′-aaagatgctgaaggagtgaaga-3′ | 22 | 914 | 341 |
| Probe | TET-5′-ccatcccctaatacgcaggatggtta-3′-TAMRA | 26 | 941 | 342 |
| Reverse | 5′-tctgagagaggagtttctccaa-3′ | 22 | 992 | 343 |

TABLE WB
CNS_neurodegeneration_v1.0

| | Rel. Exp. (%) | | Rel. Exp. (%) |
|---|---|---|---|
| | Ag3300, Run | | Ag3300, Run |
| Tissue Name | 210063573 | Tissue Name | 210063573 |
| AD 1 Hippo | 0.0 | Control (Path) 3 Temporal | 0.0 |
| | | Ctx | |
| AD 2 Hippo | 0.0 | Control (Path) 4 Temporal | 0.0 |
| | | Ctx | |
| AD 3 Hippo | 0.0 | AD 1 Occipital Ctx | 0.0 |
| AD 4 Hippo | 0.0 | AD 2 Occipital Ctx | 0.0 |
| | | (Missing) | |
| AD 5 Hippo | 0.0 | AD 3 Occipital Ctx | 0.0 |
| AD 6 Hippo | 16.6 | AD 4 Occipital Ctx | 0.0 |
| Control 2 Hippo | 7.1 | AD 5 Occipital Ctx | 0.0 |
| Control 4 Hippo | 0.0 | AD 6 Occipital Ctx | 0.0 |
| Control (Path) 3 Hippo | 0.0 | Control 1 Occipital Ctx | 0.0 |
| AD 1 Temporal Ctx | 0.0 | Control 2 Occipital Ctx | 0.0 |
| AD 2 Temporal Ctx | 0.0 | Control 3 Occipital Ctx | 38.4 |
| AD 3 Temporal Ctx | 0.0 | Control 4 Occipital Ctx | 0.0 |
| AD 4 Temporal Ctx | 0.0 | Control (Path) 1 Occipital | 100.0 |
| | | Ctx | |
| AD 5 Inf Temporal Ctx | 42.6 | Control (Path) 2 Occipital | 0.0 |
| | | Ctx | |
| AD 5 Sup Temporal Ctx | 0.0 | Control (Path) 3 Occipital | 0.0 |
| | | Ctx | |
| AD 6 Inf Temporal Ctx | 0.0 | Control (Path) 4 Occipital | 0.0 |
| | | Ctx | |
| AD 6 Sup Temporal Ctx | 0.0 | Control 1 Parietal Ctx | 39.8 |
| Control 1 Temporal Ctx | 0.0 | Control 2 Parietal Ctx | 0.0 |
| Control 2 Temporal Ctx | 0.0 | Control 3 Parietal Ctx | 0.0 |
| Control 3 Temporal Ctx | 0.0 | Control (Path) 1 Parietal | 0.0 |
| | | Ctx | |
| Control 3 Temporal Ctx | 0.0 | Control (Path) 2 Parietal | 0.0 |
| | | Ctx | |
| Control (Path) 1 Temporal | 57.0 | Control (Path) 3 Parietal | 0.0 |
| Ctx | | Ctx | |
| Control (Path) 2 Temporal | 41.8 | Control (Path) 4 Parietal | 0.0 |
| Ctx | | Ctx | |

TABLE WC
General_screening_panel_v1.4

| | Rel. Exp. (%) | Rel. Exp. (%) | | Rel. Exp. (%) | Rel. Exp. (%) |
|---|---|---|---|---|---|
| | Ag3300, Run | Ag3300, Run | | Ag3300, Run | Ag3300, Run |
| Tissue Name | 215669620 | 217235346 | Tissue Name | 215669620 | 217235346 |
| Adipose | 0.0 | 0.0 | Renal ca. TK-10 | 5.5 | 10.4 |
| Melanoma* | 0.0 | 0.0 | Bladder | 0.0 | 0.0 |
| Hs688(A).T | | | | | |
| Melanoma* | 0.0 | 0.0 | Gastric ca. (liver | 0.0 | 0.0 |
| Hs688(B).T | | | met.) NCI-N87 | | |
| Melanoma* M14 | 0.0 | 0.0 | Gastric ca. KATO III | 0.0 | 0.0 |
| Melanoma* | 0.0 | 0.0 | Colon ca. SW-948 | 0.0 | 10.2 |
| LOXIMVI | | | | | |
| Melanoma* SK- | 0.0 | 0.0 | Colon ca. SW480 | 0.0 | 0.0 |
| MEL-5 | | | | | |
| Squamous cell | 0.0 | 0.0 | Colon ca.* (SW480 | 0.0 | 0.0 |
| carcinoma SCC-4 | | | met) SW620 | | |
| Testis Pool | 4.1 | 5.6 | Colon ca. HT29 | 0.0 | 0.0 |
| Prostate ca.* | 0.0 | 7.1 | Colon ca. HCT-116 | 0.0 | 0.0 |
| (bone met) PC-3 | | | | | |
| Prostate Pool | 13.1 | 29.7 | Colon ca. CaCo-2 | 0.0 | 17.2 |
| Placenta | 0.0 | 0.0 | Colon cancer tissue | 8.7 | 18.4 |
| Uterus Pool | 0.0 | 0.0 | Colon ca. SW1116 | 0.0 | 0.0 |
| Ovarian ca. | 0.0 | 0.0 | Colon ca. Colo-205 | 0.0 | 0.0 |
| OVCAR-3 | | | | | |
| Ovarian ca. SK- | 0.0 | 0.0 | Colon ca. SW-48 | 0.0 | 0.0 |
| OV-3 | | | | | |
| Ovarian ca. | 0.0 | 0.0 | Colon Pool | 0.0 | 0.0 |
| OVCAR-4 | | | | | |
| Ovarian ca. | 19.1 | 6.7 | Small Intestine Pool | 0.0 | 5.9 |
| OVCAR-5 | | | | | |
| Ovarian ca. | 0.0 | 0.0 | Stomach Pool | 0.0 | 0.0 |
| IGROV-1 | | | | | |
| Ovarian ca. | 0.0 | 0.0 | Bone Marrow Pool | 0.0 | 0.0 |
| OVCAR-8 | | | | | |
| Ovary | 0.0 | 0.0 | Fetal Heart | 0.0 | 0.0 |
| Breast ca. MCF-7 | 7.4 | 0.0 | Heart Pool | 0.0 | 0.0 |
| Breast ca. MDA- | 0.0 | 0.0 | Lymph Node Pool | 0.0 | 0.0 |
| MB-231 | | | | | |
| Breast ca. BT 549 | 0.0 | 0.0 | Fetal Skeletal Muscle | 0.0 | 0.0 |
| Breast ca. T47D | 43.5 | 63.7 | Skeletal Muscle Pool | 0.0 | 0.0 |
| Breast ca. MDA-N | 0.0 | 0.0 | Spleen Pool | 0.0 | 0.0 |
| Breast Pool | 0.0 | 0.0 | Thymus Pool | 0.0 | 5.3 |
| Trachea | 4.4 | 0.0 | CNS cancer | 0.0 | 0.0 |
| | | | (glio/astro) U87-MG | | |
| Lung | 0.0 | 0.0 | CNS cancer | 5.4 | 5.4 |
| | | | (glio/astro) U-118- | | |
| | | | MG | | |
| Fetal Lung | 3.5 | 0.0 | CNS cancer | 0.0 | 0.0 |
| | | | (neuro; met) SK-N- | | |
| | | | AS | | |
| Lung ca. NCI- | 0.0 | 0.0 | CNS cancer (astro) | 0.0 | 0.0 |
| N417 | | | SF-539 | | |
| Lung ca. LX-1 | 0.0 | 0.0 | CNS cancer (astro) | 0.0 | 0.0 |
| | | | SNB-75 | | |
| Lung ca. NCI- | 16.0 | 0.0 | CNS cancer (glio) | 0.0 | 0.0 |
| H146 | | | SNB-19 | | |
| Lung ca. SHP-77 | 0.0 | 0.0 | CNS cancer (glio) | 0.0 | 0.0 |
| | | | SF-295 | | |
| Lung ca. A549 | 0.0 | 0.0 | Brain (Amygdala) | 6.3 | 0.0 |
| | | | Pool | | |
| Lung ca. NCI- | 100.0 | 100.0 | Brain (cerebellum) | 0.0 | 6.8 |
| H526 | | | | | |
| Lung ca. NCI-H23 | 88.3 | 0.0 | Brain (fetal) | 7.9 | 13.9 |
| Lung ca. NCI- | 34.4 | 0.0 | Brain (Hippocampus) | 0.0 | 0.0 |
| H460 | | | Pool | | |
| Lung ca. HOP-62 | 0.0 | 0.0 | Cerebral Cortex Pool | 0.0 | 0.0 |
| Lung ca. NCI- | 0.0 | 0.0 | Brain (Substantia | 0.0 | 0.0 |
| H522 | | | nigra) Pool | | |
| Liver | 0.0 | 8.2 | Brain (Thalamus) | 4.9 | 0.0 |
| | | | Pool | | |
| Fetal Liver | 15.5 | 16.5 | Brain (whole) | 10.2 | 0.0 |
| Liver ca. HepG2 | 8.0 | 7.1 | Spinal Cord Pool | 0.0 | 0.0 |
| Kidney Pool | 0.0 | 12.7 | Adrenal Gland | 0.0 | 0.0 |
| Fetal Kidney | 0.0 | 7.6 | Pituitary gland Pool | 0.0 | 0.0 |
| Renal ca. 786-0 | 0.0 | 0.0 | Salivary Gland | 19.6 | 14.3 |
| Renal ca. A498 | 39.8 | 0.0 | Thyroid (female) | 0.0 | 0.0 |
| Renal ca. ACHN | 0.0 | 0.0 | Pancreatic ca. | 0.0 | 0.0 |
| | | | CAPAN2 | | |
| Renal ca. UO-31 | 0.0 | 0.0 | Pancreas Pool | 0.0 | 0.0 |

TABLE WD
Panel 4D

| | Rel. Exp. (%) | | Rel. Exp. (%) |
|---|---|---|---|
| | Ag3300, Run | | Ag3300, Run |
| Tissue Name | 164682539 | Tissue Name | 164682539 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC none | 0.0 |
| Primary Tr1 act | 0.0 | Microsvasular Dermal EC | 0.0 |
| | | TNFalpha + IL-1beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNFalpha + | 0.0 |
| act | | IL-1beta | |
| CD8 lymphocyte act | 0.0 | Astrocytes rest | 0.0 |
| Secondary CD8 lymphocyte | 0.0 | Astrocytes TNFalpha + IL-1beta | 0.0 |
| rest | | | |
| Secondary CD8 lymphocyte | 0.0 | KU-812 (Basophil) rest | 0.0 |
| act | | | |
| CD4 lymphocyte none | 12.7 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNFalpha + IL-1beta | |
| LAK cells IL-2 | 0.0 | Liver cirrhosis | 74.2 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 31.4 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 | 0.0 |
| | | beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 16.5 | Dermal fibroblast CCD1070 rest | 0.0 |
| IL-4 | | | |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL-1 | 0.0 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 24.8 |
| Macrophages rest | 0.0 | Lung | 37.1 |
| Macrophages LPS | 0.0 | Thymus | 100.0 |
| HUVEC none | 0.0 | Kidney | 0.0 |
| HUVEC starved | 0.0 | | |

CNS_neurodegeneration_v1.0 Summary: Ag3300 Expression of this gene is low/undetectable (CTs>35) across all of the samples on this panel (data not shown).

General_screening_panel_v1.4 Summary: Ag3300 Two experiments using the same probe/primer set on this panel yielded similar results. Significant expression of the CG57662-01 gene is seen only in breast, renal, and lung cancer cell lines. Therefore, expression of this gene can be used to differentiate these lines from from the other samples on this panel. Furthermore, therapeutic modulation of the activity of this gene or its protein product, using small molecule drugs, antibodies or protein therapeutics, may be of use in the treatment of breast, renal, and lung cancer.

Panel 4D Summary: Ag3300 The CG57662-01 gene is expressed at significant levels only in the thymus (CT=34.6). The putative connexin encoded for by this gene could therefore play an important role in T cell development. Small molecule therapeutics, or antibody therapeutics designed against the GPCR encoded for by this gene could be utilized to modulate immune function (T cell development) and be important for organ transplant, AIDS treatment or post chemotherapy immune reconstitiution.

References:

1. Evans W H, Boitano S. Connexin mimetic peptides: specific inhibitors of gap-junctional intercellular communication. Biochem Soc Trans. 2001 August;29(Pt 4):606-12.

Intercellular co-operation is a fundamental and widespread feature in tissues and organs. An important mechanism ensuring multicellular homoeostasis involves signalling between cells via gap junctions that directly connect the cytosolic contents of adjacent cells. Cell proliferation and intercellular communication across gap junctions are closely linked, and a number of pathologies in which communication is disrupted are known where connexins, the gap-junctional proteins, are modified. The proteins of gap junctions thus emerge as therapeutic targets inviting the development and exploitation of chemical tools and drugs that specifically influence intercellular communication. Connexin mimetic peptides that correspond to short specific sequences in the two extracellular loops of connexins are a class of benign, specific and reversible inhibitors of gap-junctional communication that have been studied recently in a broad range of cells, tissues and organs. This review summarizes the properties and uses of these short synthetic peptides, and compares their probable mechanism of action with those of a wide range of other less specific traditional gap-junction inhibitors.

NOV29

Expression of NOV29/CG57664-01 was assessed using the primer-probe sets Ag3301 and Ag3417, described in Tables XA and XB. Results of the RTQ-PCR runs are shown in Tables XC, XD and XE.

TABLE XA
Probe Name Ag3301

| Primers | Sequences | Length | Start Position | SEQ ID NO |
|---|---|---|---|---|
| Forward | 5′-ctacgatggcaaggattacatc-3′ | 22 | 554 | 344 |
| Probe | TET-5′-ctgaacgaggacctgccctcctg-3′-TAMRA | 23 | 579 | 345 |
| Reverse | 5′-cacttgtgctgggagatctg-3′ | 20 | 624 | 346 |

TABLE XB
Probe Name Ag3417

| Primers | Sequences | Length | Start Position | SEQ ID NO |
|---|---|---|---|---|
| Forward | 5′-cgcagatttaccgagtgaac-3′ | 20 | 400 | 347 |
| Probe | TET-5′-gaccctgctccgctattacaaccaga-3′-TAMRA | 26 | 425 | 348 |
| Reverse | 5′-ctggatggtgtgagaaccac-3′ | 20 | 460 | 349 |

TABLE XC
CNS_neurodegeneration_v1.0

| | Rel. Exp. (%) | | Rel. Exp. (%) |
|---|---|---|---|
| | Ag3301, Run | | Ag3301, Run |
| Tissue Name | 210063574 | Tissue Name | 210063574 |
| AD 1 Hippo | 34.4 | Control (Path) 3 Temporal | 8.8 |
| | | Ctx | |
| AD 2 Hippo | 48.0 | Control (Path) 4 Temporal | 71.2 |
| | | Ctx | |
| AD 3 Hippo | 3.8 | AD 1 Occipital Ctx | 46.0 |
| AD 4 Hippo | 15.6 | AD 2 Occipital Ctx | 0.0 |
| | | (Missing) | |
| AD 5 Hippo | 77.4 | AD 3 Occipital Ctx | 8.3 |
| AD 6 Hippo | 100.0 | AD 4 Occipital Ctx | 36.1 |
| Control 2 Hippo | 29.1 | AD 5 Occipital Ctx | 33.4 |
| Control 4 Hippo | 33.0 | AD 6 Occipital Ctx | 25.3 |
| Control (Path) 3 Hippo | 4.6 | Control 1 Occipital Ctx | 10.6 |
| AD 1 Temporal Ctx | 33.4 | Control 2 Occipital Ctx | 41.5 |
| AD 2 Temporal Ctx | 36.6 | Control 3 Occipital Ctx | 27.2 |
| AD 3 Temporal Ctx | 7.9 | Control 4 Occipital Ctx | 10.7 |
| AD 4 Temporal Ctx | 69.3 | Control (Path) 1 Occipital | 94.6 |
| | | Ctx | |
| AD 5 Inf Temporal Ctx | 39.5 | Control (Path) 2 Occipital | 26.8 |
| | | Ctx | |
| AD 5 SupTemporal Ctx | 36.9 | Control (Path) 3 Occipital | 4.1 |
| | | Ctx | |
| AD 6 Inf Temporal Ctx | 94.0 | Control (Path) 4 Occipital | 45.4 |
| | | Ctx | |
| AD 6 Sup Temporal Ctx | 96.6 | Control 1 Parietal Ctx | 21.0 |
| Control 1 Temporal Ctx | 15.7 | Control 2 Parietal Ctx | 36.1 |
| Control 2 Temporal Ctx | 37.6 | Control 3 Parietal Ctx | 20.6 |
| Control 3 Temporal Ctx | 14.7 | Control (Path) 1 Parietal | 71.7 |
| | | Ctx | |
| Control 4 Temporal Ctx | 20.0 | Control (Path) 2 Parietal | 43.2 |
| | | Ctx | |
| Control (Path) 1 Temporal | 85.9 | Control (Path) 3 Parietal | 5.4 |
| Ctx | | Ctx | |
| Control (Path) 2 Temporal | 52.9 | Control (Path) 4 Parietal | 88.9 |
| Ctx | | Ctx | |

TABLE XD
General_screening_panel_v1.4

| | Rel. Exp. (%) | Rel. Exp. (%) | | Rel. Exp. (%) | Rel. Exp. (%) |
|---|---|---|---|---|---|
| | Ag3301, Run | Ag3301, Run | | Ag3301, Run | Ag3301, Run |
| Tissue Name | 215670031 | 217235352 | Tissue Name | 215670031 | 217235352 |
| Adipose | 7.4 | 7.9 | Renal ca. TK-10 | 1.6 | 0.9 |
| Melanoma* | 2.0 | 1.8 | Bladder | 18.3 | 20.7 |
| Hs688(A).T | | | | | |
| Melanoma* | 1.3 | 1.8 | Gastric ca. (liver | 57.0 | 52.5 |
| Hs688(B).T | | | met.) NCI-N87 | | |
| Melanoma* M14 | 0.2 | 0.4 | Gastric ca. KATO III | 25.5 | 27.7 |
| Melanoma* | 5.9 | 4.0 | Colon ca. SW-948 | 4.6 | 4.8 |
| LOXIMVI | | | | | |
| Melanoma* SK- | 3.3 | 4.0 | Colon ca. SW480 | 7.9 | 6.9 |
| MEL-5 | | | | | |
| Squamous cell | 1.4 | 1.4 | Colon ca.* (SW480 | 3.6 | 3.1 |
| carcinoma SCC-4 | | | met) SW620 | | |
| Testis Pool | 2.3 | 1.5 | Colon ca. HT29 | 0.0 | 0.0 |
| Prostate ca.* | 5.0 | 4.2 | Colon ca. HCT-116 | 3.2 | 3.4 |
| (bone met) PC-3 | | | | | |
| Prostate Pool | 4.5 | 4.6 | Colon ca. CaCo-2 | 0.4 | 0.5 |
| Placenta | 4.4 | 4.4 | Colon cancer tissue | 87.7 | 100.0 |
| Uterus Pool | 1.8 | 1.7 | Colon ca. SW1116 | 2.3 | 3.5 |
| Ovarian ca. | 8.6 | 8.4 | Colon ca. Colo-205 | 8.8 | 6.7 |
| OVCAR-3 | | | | | |
| Ovarian ca. SK- | 2.5 | 4.3 | Colon ca. SW-48 | 25.0 | 16.8 |
| OV-3 | | | | | |
| Ovarian ca. | 8.8 | 9.1 | Colon Pool | 6.8 | 7.5 |
| OVCAR-4 | | | | | |
| Ovarian ca. | 32.5 | 20.6 | Small Intestine Pool | 6.8 | 6.7 |
| OVCAR-5 | | | | | |
| Ovarian ca. | 6.2 | 4.2 | Stomach Pool | 5.3 | 7.6 |
| IGROV-1 | | | | | |
| Ovarian ca. | 24.8 | 30.8 | Bone Marrow Pool | 1.9 | 1.7 |
| OVCAR-8 | | | | | |
| Ovary | 9.1 | 7.7 | Fetal Heart | 1.4 | 0.9 |
| Breast ca. MCF-7 | 0.4 | 0.3 | Heart Pool | 2.9 | 4.4 |
| Breast ca. MDA- | 100.0 | 73.2 | Lymph Node Pool | 8.6 | 9.8 |
| MB-231 | | | | | |
| Breast ca. BT 549 | 8.5 | 9.3 | Fetal Skeletal Muscle | 0.6 | 0.4 |
| Breast ca. T47D | 86.5 | 93.3 | Skeletal Muscle Pool | 3.5 | 4.1 |
| Breast ca. MDA-N | 0.3 | 0.3 | Spleen Pool | 24.5 | 31.9 |
| Breast Pool | 7.5 | 6.2 | Thymus Pool | 13.2 | 11.7 |
| Trachea | 15.5 | 13.6 | CNS cancer | 0.1 | 0.2 |
| | | | (glio/astro) U87-MG | | |
| Lung | 0.9 | 1.1 | CNS cancer | 8.9 | 10.7 |
| | | | (glio/astro) U-118- | | |
| | | | MG | | |
| Fetal Lung | 8.1 | 9.2 | CNS cancer | 5.8 | 4.9 |
| | | | (neuro; met) SK-N- | | |
| | | | AS | | |
| Lung ca. NCI- | 0.1 | 0.2 | CNS cancer (astro) | 14.4 | 18.3 |
| N417 | | | SF-539 | | |
| Lung ca. LX-1 | 6.0 | 6.8 | CNS cancer (astro) | 21.5 | 34.4 |
| | | | SNB-75 | | |
| Lung ca. NCI- | 0.8 | 0.4 | CNS cancer (glio) | 6.3 | 5.0 |
| H146 | | | SNB-19 | | |
| Lung ca. SHP-77 | 0.2 | 0.3 | CNS cancer (glio) | 22.4 | 17.9 |
| | | | SF-295 | | |
| Lung ca. A549 | 4.4 | 3.8 | Brain (Amygdala) | 4.5 | 3.5 |
| | | | Pool | | |
| Lung ca. NCI- | 15.3 | 12.8 | Brain (cerebellum) | 7.3 | 5.4 |
| H526 | | | | | |
| Lung ca. NCI-H23 | 15.1 | 9.4 | Brain (fetal) | 4.9 | 5.6 |
| Lung ca. NCI- | 10.7 | 9.8 | Brain (Hippocampus) | 4.1 | 3.6 |
| H460 | | | Pool | | |
| Lung ca. HOP-62 | 20.6 | 18.3 | Cerebral Cortex Pool | 4.2 | 3.8 |
| Lung ca. NCI- | 2.4 | 1.3 | Brain (Substantia | 6.6 | 6.3 |
| H522 | | | nigra) Pool | | |
| Liver | 1.9 | 1.5 | Brain (Thalamus) | 4.9 | 6.0 |
| | | | Pool | | |
| Fetal Liver | 2.0 | 1.8 | Brain (whole) | 3.8 | 4.7 |
| Liver ca. HepG2 | 1.2 | 1.2 | Spinal Cord Pool | 3.0 | 3.7 |
| Kidney Pool | 15.0 | 16.7 | Adrenal Gland | 7.3 | 7.9 |
| Fetal Kidney | 2.6 | 2.6 | Pituitary gland Pool | 2.7 | 2.3 |
| Renal ca. 786-0 | 6.4 | 5.6 | Salivary Gland | 4.0 | 3.6 |
| Renal ca. A498 | 11.7 | 10.6 | Thyroid (female) | 4.2 | 4.0 |
| Renal ca. ACHN | 4.2 | 2.6 | Pancreatic ca. | 13.6 | 9.6 |
| | | | CAPAN2 | | |
| Renal ca. UO-31 | 7.9 | 4.7 | Pancreas Pool | 11.9 | 13.6 |

TABLE XE
Panel 4D

| | Rel. Exp. (%) | Rel. Exp. (%) | | Rel. Exp. (%) | Rel. Exp. (%) |
|---|---|---|---|---|---|
| | Ag3301, Run | Ag3417, Run | | Ag3301, Run | Ag3417, Run |
| Tissue Name | 164682541 | 166453854 | Tissue Name | 164682541 | 166453854 |
| Secondary Th1 act | 13.6 | 0.0 | HUVEC IL-1beta | 0.2 | 0.0 |
| Secondary Th2 act | 44.4 | 0.0 | HUVEC IFN gamma | 5.9 | 100.0 |
| Secondary Tr1 act | 33.9 | 0.0 | HUVEC TNF alpha + | 0.4 | 0.0 |
| | | | IFN gamma | | |
| Secondary Th1 rest | 31.0 | 0.0 | HUVEC TNF alpha + | 0.2 | 0.0 |
| | | | IL4 | | |
| Secondary Th2 rest | 22.4 | 0.0 | HUVEC IL-11 | 1.9 | 0.0 |
| Secondary Tr1 rest | 34.9 | 0.0 | Lung Microvascular EC | 8.5 | 0.0 |
| | | | none | | |
| Primary Th1 act | 21.5 | 0.0 | Lung Microvascular EC | 13.4 | 0.0 |
| | | | TNF alpha + IL-1beta | | |
| Primary Th2 act | 22.5 | 0.0 | Microvascular Dermal | 6.0 | 0.0 |
| | | | EC none | | |
| Primary Tr1 act | 33.9 | 0.0 | Microsvasular Dermal | 22.5 | 0.0 |
| | | | EC TNF alpha + IL- | | |
| | | | 1beta | | |
| Primary Th1 rest | 37.4 | 0.0 | Bronchial epithelium | 16.8 | 0.0 |
| | | | TNF alpha + IL1beta | | |
| Primary Th2 rest | 0.0 | 0.0 | Small airway epithelium | 0.0 | 0.0 |
| | | | none | | |
| Primary Tr1 rest | 17.2 | 0.0 | Small airway epithelium | 0.7 | 0.0 |
| | | | TNF alpha + IL-1beta | | |
| CD45RA CD4 | 10.6 | 0.0 | Coronery artery SMC | 1.1 | 0.0 |
| lymphocyte act | | | rest | | |
| CD45RO CD4 | 29.3 | 0.0 | Coronery artery SMC | 0.3 | 0.0 |
| lymphocyte act | | | TNF alpha + IL-1beta | | |
| CD8 lymphocyte act | 29.3 | 0.0 | Astrocytes rest | 8.1 | 0.0 |
| Secondary CD8 | 48.3 | 0.0 | Astrocytes TNF alpha + | 7.0 | 0.0 |
| lymphocyte rest | | | IL-1beta | | |
| Secondary CD8 | 2.4 | 0.0 | KU-812 (Basophil) rest | 0.8 | 0.0 |
| lymphocyte act | | | | | |
| CD4 lymphocyte none | 13.6 | 0.0 | KU-812 (Basophil) | 2.4 | 0.0 |
| | | | PMA/ionomycin | | |
| 2ry Th1/Th2/Tr1_anti- | 17.3 | 0.0 | CCD1106 | 4.1 | 0.0 |
| CD95 CH11 | | | (Keratinocytes) none | | |
| LAK cells rest | 100.0 | 0.0 | CCD1106 | 12.0 | 0.0 |
| | | | (Keratinocytes) | | |
| | | | TNF alpha + IL-1beta | | |
| LAK cells IL-2 | 54.7 | 0.0 | Liver cirrhosis | 8.4 | 0.0 |
| LAK cells IL-2 + IL-12 | 49.7 | 0.0 | Lupus kidney | 0.4 | 0.0 |
| LAK cells IL-2 + IFN | 44.8 | 0.0 | NCI-H292 none | 2.3 | 0.0 |
| gamma | | | | | |
| LAK cells IL-2 + IL- | 34.2 | 0.0 | NCI-H292 IL-4 | 3.6 | 0.0 |
| 18 | | | | | |
| LAK cells | 80.1 | 0.0 | NCI-H292 IL-9 | 2.6 | 0.0 |
| PMA/ionomycin | | | | | |
| NK Cells IL-2 rest | 24.1 | 0.0 | NCI-H292 IL-13 | 1.4 | 0.0 |
| Two Way MLR 3 day | 58.6 | 0.0 | NCI-H292 IFN gamma | 1.4 | 0.0 |
| Two Way MLR 5 day | 25.9 | 0.0 | HPAEC none | 6.0 | 0.0 |
| Two Way MLR 7 day | 6.6 | 0.0 | HPAEC TNF alpha + | 23.7 | 0.0 |
| | | | IL-1beta | | |
| PBMC rest | 14.5 | 0.0 | Lung fibroblast none | 0.7 | 0.0 |
| PBMC PWM | 35.4 | 0.0 | Lung fibroblast TNF | 0.1 | 0.0 |
| | | | alpha + IL-1beta | | |
| PBMC PHA-L | 24.8 | 0.0 | Lung fibroblast IL-4 | 0.5 | 0.0 |
| Ramos (B cell) none | 8.4 | 0.0 | Lung fibroblast IL-9 | 0.4 | 0.0 |
| Ramos (B cell) | 14.7 | 0.0 | Lung fibroblast IL-13 | 0.5 | 0.0 |
| ionomycin | | | | | |
| B lymphocytes PWM | 12.9 | 0.0 | Lung fibroblast IFN | 1.5 | 0.0 |
| | | | gamma | | |
| B lymphocytes | 18.4 | 0.0 | Dermal fibroblast | 12.5 | 0.0 |
| CD40L and IL-4 | | | CCD1070 rest | | |
| EOL-1 dbcAMP | 0.8 | 0.0 | Dermal fibroblast | 8.2 | 0.0 |
| | | | CCD1070 TNF alpha | | |
| EOL-1 dbcAMP | 0.3 | 0.0 | Dermal fibroblast | 6.5 | 0.0 |
| PMA/ionomycin | | | CCD1070 IL-1beta | | |
| Dendritic cells none | 59.0 | 0.0 | Dermal fibroblast IFN | 34.6 | 0.0 |
| | | | gamma | | |
| Dendritic cells LPS | 10.8 | 0.0 | Dermal fibroblast IL-4 | 9.7 | 0.0 |
| Dendritic cells anti- | 16.8 | 0.0 | IBD Colitis 2 | 1.2 | 0.0 |
| CD40 | | | | | |
| Monocytes rest | 36.1 | 0.0 | IBD Crohn's | 0.2 | 0.0 |
| Monocytes LPS | 30.1 | 0.0 | Colon | 27.2 | 0.0 |
| Macrophages rest | 17.3 | 0.0 | Lung | 6.1 | 0.0 |
| Macrophages LPS | 57.4 | 0.0 | Thymus | 8.4 | 0.0 |
| HUVEC none | 0.2 | 0.0 | Kidney | 33.7 | 0.0 |
| HUVEC starved | 0.4 | 0.0 | | | |

CNS_neurodegeneration_v1.0 Summary: Ag3301 This panel confirms the expression of this gene at low to moderate levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders. Ag3417 Expression of this gene is low/undetectable (CTs>35) across all of the samples on this panel (data not shown).

General_screening_panel_v1.4 Summary: Ag3301 Results from two experiments with using the same probe/primer set yielded results that are in excellent agreement. Expression of the CG57664-01 gene is moderate to high across the majority of samples on this panel, with highest expression in a colon cancer tissue (CT=26.8). This gene appears to be more highly expressed in CNS, colon, gastric, renal, lung, ovarian, breast, and melanoma cancer cell lines when compared to normal tissues. The CG57664-01 gene encodes a protein with homology to MHC class I antigen. The major histocompatibility complex (MHC) class I family of glycoproteins presents peptides for immunorecognition by cytotoxic T lymphocytes. Increased expression of this gene in cancer cell lines agrees with the results of other studies that found elavated levels of LILA class I antigen in tumors (ref. 1). Therefore, reduction in the expression or activity of this novel HLA class I antigen may make cancer cells sensitive to lysis by natural killer cells.

In addition, this gene is expressed at moderate levels in all regions of the central nervous system examined including amygdala, hippocampus, substantia nigra, cerebellum, cerebral cortex, thalamus and spinal cord. Class I MHC molecules, known to be important for immune responses to antigen, are expressed also by neurons that undergo activity-dependent, long-term structural and synaptic modifications (ref. 2). Specific class I MHC mRNAs are expressed by distinct mosaics of neurons, reflecting a potential for diverse neuronal functions and suggesting an important role for these molecules in the activity-dependent remodeling and plasticity of connections in the developing and mature mammalian central nervous system.

Among tissues with metabolic or endocrine function, this gene is expressed at moderate levels in adrenal gland, thyroid, pituitary gland, pancreas, adipose, skeletal muscle, heart and liver. Therefore, therapeutic modulation of the activity of this gene may prove useful in the treatment of endocrine/metabolically related diseases, such as obesity and diabetes.

References:

1. Sette A, Chesnut R, Fikes J. HLA expression in cancer: implications for T cell-based immunotherapy. Immunogenetics 2001 May-June;53(4):255-63.

HLA class I expression is altered in a significant fraction of the tumor types reviewed here, reflecting either immune pressure or, simply, the accumulation of pathological changes and alterations. However, in all tumor types analyzed, a majority of the tumors express HLA class I. with a general tendency for the more severe alterations to be found in later-stage and less differentiated tumors. These results are encouraging for the development of specific immunotherapies, especially considering that (1) the relatively low sensitivity of immunohistochemical techniques might underestimate HLA expression in tumors, (2) class I expression can be induced in tumor cells as a result of local inflammation and lymphokine release, and (3) class I-negative cells would be predicted to be sensitive to lysis by natural killer cells.

PMID: 11491528

2. Huh G S, Boulanger L M, Du H, Riquelme P A, Brotz T M, Shatz C J. Functional requirement for class I MHC in CNS development and plasticity. Science Dec. 15, 2000;290(5499):2155-9

Class I major histocompatibility complex (class I MHC) molecules, known to be important for immune responses to antigen, are expressed also by neurons that undergo activity-dependent, long-term structural and synaptic modifications. Here, we show that in mice genetically deficient for cell surface class I MHC or for a class I MHC receptor component, CD3zeta, refinement of connections between retina and central targets during development is incomplete. In the hippocampus of adult mutants, N-methyl-D aspartate receptor-dependent long-term potentiation (LTP) is enhanced, and long-term depression (LTD) is absent. Specific class I MHC messenger RNAs are expressed by distinct mosaics of neurons, reflecting a potential for diverse neuronal functions. These results demonstrate an important role for these molecules in the activity dependent remodeling and plasticity of connections in the developing and mature mammalian central nervous system (CNS).

PMID: 11118151

Panel 4D Summary: Ag3301 The CG57664-01 gene is moderately expressed in a number of samples on this panel, with highest expression in lymphokine-activated killer (LAK) cells (CT=26-28), independent of stimulation. This observation suggests that modulation of the expression or activity of this gene may be useful in the treatment of intracellular bacterial or viral infections. This gene is also expressed at significant levels in T cells, B cells, dendritic cells, monocytes and macrophages.

The CG57664-01 gene encodes a protein with homology to MHC class I antigen. Major histocompatibility complex (MHC) class I molecules present antigenic peptides to CD8-positive T cells. The HLA class I gene family in humans consists of 6 members: polymorphic HLA-A, HLA-B, and HLA-C, and oligomorphic HLA-E, HLA-F, and HLA-G. HLA-A/B-alleles such as the CG57664-01 gene may have utility in genotyping criminals as well as in paternity disputes. These antigens are ubiquitously expressed on all nucleated human cells except neurons and trophoblasts, and participate in antigen presentation of viral antigens in the adaptive immune response.

Ag3417 Results from one experiment with the CG57664-01 gene are not included. The amp plot indicates that there were experimental difficulties with this run.

NOV30

Expression of NOV30/CG57666-01 was assessed using the primer-probe set Ag3302, described in Table YA.

TABLE YA
Probe Name Ag3302

| Primers | Sequences | Length | Start Position | SEQ ID NO |
|---|---|---|---|---|
| Forward | 5′-gtgatgtgcaggaagaactcat-3′ | 22 | 1056 | 350 |
| Probe | TET-5′-tttgttctaccccaggcagcaaccat-3′-TAMRA | 26 | 1078 | 351 |
| Reverse | 5′-ttacaagccgtgagagacaca-3′ | 21 | 1118 | 352 |

CNS_neurodegeneration_v1.0 Summary: Ag3302 Expression of the CG57666-01 gene is low/undetectable (CTs>35) across all of the samples on this panel (data not shown).

General_screening_panel_v1.4 Summary: Ag3302 Expression of the CG57666-01 gene is low/undetectable (CTs>35) across all of the samples on this panel (data not shown).

Panel 4D Summary: Ag3302 Expression of the CG57666-01 gene is low/undetectable (CTs>35) across all of the samples on this panel (data not shown).

NOV31

Expression of NOV31/CG57668-01 was assessed using the primer-probe set Ag3303, described in Table ZA. Results of the RTQ-PCR runs are shown in Tables ZB, ZC and ZD.

TABLE ZA
Probe Name Ag3303

| Primers | Sequences | Length | Start Position | SEQ ID NO |
|---|---|---|---|---|
| Forward | 5′-ctacgacggcaaggattacat-3′ | 21 | 438 | 353 |
| Probe | TET-5′-ctctgaacgaggacctgcgctcct-3′-TAMRA | 24 | 461 | 354 |
| Reverse | 5′-gtgatctgagctgccatgtc-3′ | 20 | 496 | 355 |

TABLE ZB
CNS_neurodegeneration_v1.0

| | Rel. Exp. (%) | | Rel. Exp. (%) |
|---|---|---|---|
| | Ag3303, Run | | Ag3303, Run |
| Tissue Name | 210063655 | Tissue Name | 210063655 |
| AD 1 Hippo | 27.4 | Control (Path) 3 Temporal | 16.6 |
| | | Ctx | |
| AD 2 Hippo | 55.9 | Control (Path) 4 Temporal | 14.5 |
| | | Ctx | |
| AD 3 Hippo | 14.3 | AD 1 Occipital Ctx | 16.3 |
| AD 4 Hippo | 35.6 | AD 2 Occipital Ctx | 0.0 |
| | | (Missing) | |
| AD 5 Hippo | 96.6 | AD 3 Occipital Ctx | 21.8 |
| AD 6 Hippo | 12.7 | AD 4 Occipital Ctx | 29.3 |
| Control 2 Hippo | 30.4 | AD 5 Occipital Ctx | 3.0 |
| Control 4 Hippo | 94.6 | AD 6 Occipital Ctx | 37.4 |
| Control (Path) 3 Hippo | 15.3 | Control 1 Occipital Ctx | 16.5 |
| AD 1 Temporal Ctx | 18.2 | Control 2 Occipital Ctx | 47.0 |
| AD 2 Temporal Ctx | 34.4 | Control 3 Occipital Ctx | 18.0 |
| AD 3 Temporal Ctx | 17.9 | Control 4 Occipital Ctx | 37.6 |
| AD 4 Temporal Ctx | 31.9 | Control (Path) 1 Occipital | 18.4 |
| | | Ctx | |
| AD 5 Inf Temporal Ctx | 100.0 | Control (Path) 2 Occipital | 13.9 |
| | | Ctx | |
| AD 5 Sup Temporal Ctx | 100.0 | Control (Path) 3 Occipital | 12.3 |
| | | Ctx | |
| AD 6 Inf Temporal Ctx | 6.5 | Control (Path) 4 Occipital | 13.4 |
| | | Ctx | |
| AD 6 Sup Temporal Ctx | 11.0 | Control 1 Parietal Ctx | 18.2 |
| Control 1 Temporal Ctx | 19.6 | Control 2 Parietal Ctx | 72.7 |
| Control 2 Temporal Ctx | 40.6 | Control 3 Parietal Ctx | 21.3 |
| Control 3 Temporal Ctx | 20.6 | Control (Path) 1 Parietal | 15.2 |
| | | Ctx | |
| Control 4 Temporal Ctx | 61.1 | Control (Path) 2 Parietal | 22.2 |
| | | Ctx | |
| Control (Path) 1 Temporal | 14.0 | Control (Path) 3 Parietal | 11.6 |
| Ctx | | Ctx | |
| Control (Path) 2 Temporal | 31.6 | Control (Path) 4 Parietal | 18.2 |
| Ctx | | Ctx | |

TABLE ZC
General_screening_panel_v1.4

| | Rel. Exp. (%) | | Rel. Exp. (%) |
|---|---|---|---|
| | Ag3303, Run | | Ag3303, Run |
| Tissue Name | 215648467 | Tissue Name | 215648467 |
| Adipose | 0.1 | Renal ca. TK-10 | 2.1 |
| Melanoma* Hs688(A).T | 20.7 | Bladder | 0.4 |
| Melanoma* Hs688(B).T | 15.5 | Gastric ca. (liver met.) NCI- | 100.0 |
| | | N87 | |
| Melanoma* M14 | 4.2 | Gastric ca. KATO III | 0.2 |
| Melanoma* LOXIMVI | 4.2 | Colon ca. SW-948 | 6.2 |
| Melanoma* SK-MEL-5 | 8.0 | Colon ca. SW480 | 0.3 |
| Squamous cell carcinoma | 0.2 | Colon ca.* (SW480 met) | 0.1 |
| SCC-4 | | SW620 | |
| Testis Pool | 2.3 | Colon ca. HT29 | 2.8 |
| Prostate ca.* (bone met) | 5.5 | Colon ca. HCT-116 | 9.8 |
| PC-3 | | | |
| Prostate Pool | 1.5 | Colon ca. CaCo-2 | 0.4 |
| Placenta | 2.9 | Colon cancer tissue | 18.0 |
| Uterus Pool | 1.9 | Colon ca. SW1116 | 0.1 |
| Ovarian ca. OVCAR-3 | 0.2 | Colon ca. Colo-205 | 5.5 |
| Ovarian ca. SK-OV-3 | 23.5 | Colon ca. SW-48 | 19.9 |
| Ovarian ca. OVCAR-4 | 4.2 | Colon Pool | 2.0 |
| Ovarian ca. OVCAR-5 | 12.1 | Small Intestine Pool | 3.3 |
| Ovarian ca. IGROV-1 | 0.2 | Stomach Pool | 4.6 |
| Ovarian ca. OVCAR-8 | 28.5 | Bone Marrow Pool | 2.8 |
| Ovary | 4.6 | Fetal Heart | 4.4 |
| Breast ca. MCF-7 | 0.0 | Heart Pool | 3.5 |
| Breast ca. MDA-MB-231 | 1.0 | Lymph Node Pool | 9.4 |
| Breast ca. BT 549 | 4.1 | Fetal Skeletal Muscle | 0.3 |
| Breast ca. T47D | 63.7 | Skeletal Muscle Pool | 3.5 |
| Breast ca. MDA-N | 3.2 | Spleen Pool | 21.3 |
| Breast Pool | 1.9 | Thymus Pool | 10.9 |
| Trachea | 14.8 | CNS cancer (glio/astro) U87- | 0.1 |
| | | MG | |
| Lung | 1.5 | CNS cancer (glio/astro) U- | 1.2 |
| | | 118-MG | |
| Fetal Lung | 14.0 | CNS cancer (neuro; met) SK- | 0.6 |
| | | N-AS | |
| Lung ca. NCI-N417 | 0.5 | CNS cancer (astro) SF-539 | 0.3 |
| Lung ca. LX-1 | 0.0 | CNS cancer (astro) SNB-75 | 65.5 |
| Lung ca. NCI-H146 | 5.3 | CNS cancer (glio) SNB-19 | 0.3 |
| Lung ca. SHP-77 | 0.0 | CNS cancer (glio) SF-295 | 71.2 |
| Lung ca. A549 | 2.5 | Brain (Amygdala) Pool | 1.7 |
| Lung ca. NCI-H526 | 5.8 | Brain (cerebellum) | 2.9 |
| Lung ca. NCI-H23 | 11.7 | Brain (fetal) | 1.6 |
| Lung ca. NCI-H460 | 0.0 | Brain (Hippocampus) Pool | 0.4 |
| Lung ca. HOP-62 | 21.2 | Cerebral Cortex Pool | 0.4 |
| Lung ca. NCI-H522 | 0.0 | Brain (Substantia nigra) Pool | 0.8 |
| Liver | 4.0 | Brain (Thalamus) Pool | 1.0 |
| Fetal Liver | 1.7 | Brain (whole) | 1.1 |
| Liver ca. HepG2 | 0.0 | Spinal Cord Pool | 1.4 |
| Kidney Pool | 9.6 | Adrenal Gland | 14.0 |
| Fetal Kidney | 2.2 | Pituitary gland Pool | 4.1 |
| Renal ca. 786-0 | 17.8 | Salivary Gland | 2.8 |
| Renal ca. A498 | 0.0 | Thyroid (female) | 3.1 |
| Renal ca. ACHN | 2.8 | Pancreatic ca. CAPAN2 | 4.0 |
| Renal ca. UO-31 | 28.7 | Pancreas Pool | 8.2 |

TABLE ZD
Panel 4D

| | Rel. Exp. (%) | | Rel. Exp. (%) |
|---|---|---|---|
| | Ag3303, Run | | Ag3303, Run |
| Tissue Name | 164682641 | Tissue Name | 164682641 |
| Secondary Th1 act | 38.4 | HUVEC IL-1beta | 1.7 |
| Secondary Th2 act | 63.7 | HUVEC IFN gamma | 6.2 |
| Secondary Tr1 act | 52.1 | HUVEC TNF alpha + IFN gamma | 7.6 |
| Secondary Th1 rest | 1.5 | HUVEC TNF alpha + IL4 | 3.2 |
| Secondary Th2 rest | 2.0 | HUVEC IL-11 | 3.0 |
| Secondary Tr1 rest | 3.0 | Lung Microvascular EC none | 26.6 |
| Primary Th1 act | 2.7 | Lung Microvascular EC | 41.5 |
| | | TNFalpha + IL-1beta | |
| Primary Th2 act | 4.7 | Microvascular Dermal EC none | 18.9 |
| Primary Tr1 act | 6.0 | Microsvasular Dermal EC | 38.2 |
| | | TNFalpha +IL-1beta | |
| Primary Th1 rest | 81.2 | Bronchial epithelium TNFalpha + | 27.7 |
| | | IL1beta | |
| Primary Th2 rest | 50.7 | Small airway epithelium none | 10.9 |
| Primary Tr1 rest | 33.0 | Small airway epithelium | 24.3 |
| | | TNFalpha + IL-1beta | |
| CD45RA CD4 lymphocyte | 28.7 | Coronery artery SMC rest | 2.9 |
| act | | | |
| CD45RO CD4 lymphocyte | 44.4 | Coronery artery SMC TNFalpha + | 2.8 |
| act | | IL-1beta | |
| CD8 lymphocyte act | 26.8 | Astrocytes rest | 0.9 |
| Secondary CD8 lymphocyte | 44.4 | Astrocytes TNFalpha + IL-1 beta | 1.2 |
| rest | | | |
| Secondary CD8 lymphocyte | 9.6 | KU-812 (Basophil) rest | 0.6 |
| act | | | |
| CD4 lymphocyte none | 17.0 | KU-812 (Basophil) | 1.7 |
| | | PMA/ionomycin | |
| 2ry Th1/Th2/Tr1_anti-CD95 | 33.0 | CCD1106 (Keratinocytes) none | 11.7 |
| CH11 | | | |
| LAK cells rest | 87.7 | CCD1106 (Keratinocytes) | 28.9 |
| | | TNFalpha + IL-1beta | |
| LAK cells IL-2 | 60.3 | Liver cirrhosis | 3.4 |
| LAK cells IL-2 + IL-12 | 60.3 | Lupus kidney | 1.0 |
| LAK cells IL-2 + IFN gamma | 77.9 | NCI-H292 none | 2.0 |
| LAK cells IL-2 + IL-18 | 66.0 | NCI-H292 IL-4 | 3.3 |
| LAK cells PMA/ionomycin | 100.0 | NCI-H292 IL-9 | 3.0 |
| NK Cells IL-2 rest | 45.1 | NCI-H292 IL-13 | 3.0 |
| Two Way MLR 3 day | 98.6 | NCI-H292 IFN gamma | 6.0 |
| Two Way MLR 5 day | 67.4 | HPAEC none | 1.8 |
| Two Way MLR 7 day | 20.0 | HPAEC TNF alpha + IL-1 beta | 9.5 |
| PBMC rest | 30.1 | Lung fibroblast none | 21.3 |
| PBMC PWM | 82.9 | Lung fibroblast TNF alpha + IL-1 | 59.0 |
| | | beta | |
| PBMC PHA-L | 52.5 | Lung fibroblast IL-4 | 28.1 |
| Ramos (B cell) none | 29.1 | Lung fibroblast IL-9 | 22.4 |
| Ramos (B cell) ionomycin | 45.4 | Lung fibroblast IL-13 | 20.7 |
| B lymphocytes PWM | 24.1 | Lung fibroblast IFN gamma | 40.1 |
| B lymphocytes CD40L and | 12.4 | Dermal fibroblast CCD1070 rest | 17.8 |
| IL-4 | | | |
| EOL-1 dbcAMP | 1.5 | Dermal fibroblast CCD1070 TNF | 24.1 |
| | | alpha | |
| EOL-1 dbcAMP | 1.1 | Dermal fibroblast CCD1070 IL-1 | 14.5 |
| PMA/ionomycin | | beta | |
| Dendritic cells none | 56.6 | Dermal fibroblast IFN gamma | 2.7 |
| Dendritic cells LPS | 29.9 | Dermal fibroblast IL-4 | 1.4 |
| Dendritic cells anti-CD40 | 41.8 | IBD Colitis 2 | 2.7 |
| Monocytes rest | 53.6 | IBD Crohn's | 8.2 |
| Monocytes LPS | 66.9 | Colon | 5.6 |
| Macrophages rest | 88.9 | Lung | 46.0 |
| Macrophages LPS | 82.4 | Thymus | 10.7 |
| HUVEC none | 1.0 | Kidney | 34.9 |
| HUVEC starved | 2.5 | | |

CNS_neurodegeneration_v1.0 Summary: Ag3303 This panel confirms the expression of this gene at moderate to high levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3303 Expression of the CG57668-01 gene is moderate to high across the majority of samples on this panel, with highest expression in a, gastric cancer cell line (CT=24.8). This gene appears to be more highly expressed in CNS, colon, gastric, renal, lung, ovarian, breast, and melanoma cancer cell lines when compared to normal tissues. The CG57668-01 gene encodes a protein that is highly homologous to HLA class I histocompatibility antigen, alpha chain FI precursor (HLA-AR). The major histocompatibility complex (MHC) class I family of glycoproteins presents peptides for immunorecognition by cytotoxic T lymphocytes. Increased expression of this gene in cancer cell lines agrees with the results of other studies that found elavated levels of HLA class I antigen in tumors (ref. I). Therefore, reduction in the expression or activity of this novel HLA class I antigen may make cancer cells sensitive to lysis by natural killer cells.

In addition, this gene is expressed at moderate levels in all regions of the central nervous system examined including amygdala, hippocampus, substantia nigra, cerebellum, cerebral cortex, thalamus and spinal cord. Class I MHC molecules, known to be important for immune responses to antigen, are expressed also by neurons that undergo activity-dependent, long-term structural and synaptic modifications (ref. 2). Specific class I MHC mRNAs are expressed by distinct mosaics of neurons, reflecting a potential for diverse neuronal functions and suggesting an important role for these molecules in the activity-dependent remodeling and plasticity of connections in the developing and mature mammalian central nervous system.

Among tissues with metabolic or endocrine function, this gene is expressed at moderate levels in adrenal gland, thyroid, pituitary gland, pancreas, skeletal muscle, heart and liver. Therefore, therapeutic modulation of the activity of this gene may prove useful in the treatment of endocrine/metabolically related diseases, such as obesity and diabetes. In addition, there are significant differences of expression in fetal lung(CT=27.8) and adult lung (CT=30.8) as well as fetal skeletal muscle (CT=33.2) and adult skeletal muscle (CT=29.6). Therefore expression of this gene can be used to differentiate between fetal and adult lung as well as fetal and adult skeletal muscle.

References:

1. Sette A, Chesnut R, Fikes J. HLA expression in cancer: implications for T cell-based immunotherapy. Immunogenetics 2001 May-June;53(4):255-63.

HLA class I expression is altered in a significant fraction of the tumor types reviewed here, reflecting either immune pressure or, simply, the accumulation of pathological changes and alterations. However, in all tumor types analyzed, a majority of the tumors express HLA class I. with a general tendency for the more severe alterations to be found in later-stage and less differentiated tumors. These results are encouraging for the development of specific immunotherapies, especially considering that (1) the relatively low sensitivity of immunohistochemical techniques might underestimate HLA expression in tumors, (2) class I expression can be induced in tumor cells as a result of local inflammation and lymphokine release, and (3) class l-negative cells would be predicted to be sensitive to lysis by natural killer cells.

PMID: 11491528

2. Huh G S, Boulanger L M, Du H, Riquelme P A, Brotz T M, Shatz CJ. Functional requirement for class I MHC in CNS development and plasticity. Science Dec. 15, 2000;290(5499):2155-9

Class I major histocompatibility complex (class I MHC) molecules, known to be important for immune responses to antigen, are expressed also by neurons that undergo activity-dependent, long-term structural and synaptic modifications. Here, we show that in mice genetically deficient for cell surface class I MHC or for a class I MHC receptor component, CD3zeta, refinement of connections between retina and central targets during development is incomplete. In the hippocampus of adult mutants, N-methyl-D aspartate receptor-dependent long-term potentiation (LTP) is enhanced, and long-term depression (LTD) is absent. Specific class I MHC messenger RNAs are expressed by distinct mosaics of neurons, reflecting a potential for diverse neuronal functions. These results demonstrate an important role for these molecules in the activity dependent remodeling and plasticity of connections in the developing and mature mammalian central nervous system (CNS).

PMID: 11118151

Panel 4D Summary: Ag3303 The CG57668-01 gene is moderately expressed in a number of samples on this panel, with highest expression in lymphokine-activated killer (LAK) cells (CT=26-28), independent of stimulation. This observation suggests that modulation of the expression or activity of this gene may be useful in the treatment of intracellular bacterial or viral infections. This gene is also expressed at significant levels in r cells, B cells, dendritic cells, monocytes and macrophages.

The CG57668-01 gene encodes a protein with homology to MHC class I antigen. Major histocompatibility complex (MHC) class I molecules present antigenic peptides to CD8-positive T cells. The HLA class I gene family in humans consists of 6 members: polymorphic HLA-A, HLA-B, and HLA-C, and oligomorphic HLA-E, HLA-F, and HLA-G. HLA-A/B-alleles such as the CG57664-01 gene may have utility in genotyping criminals as well as in paternity disputes. These antigens are ubiquitously expressed on all nucleated human cells except neurons and trophoblasts, and participate in antigen presentation of viral antigens in the adaptive immune response.

NOV33

Expression of NOV33/CG57672-01 was assessed using the primer-probe set Ag3305, described in Table AAA. Results of the RTQ-PCR runs are shown in Tables AAB, AAC, and AAD.

TABLE AAA
Probe Name Ag3305

| Primers | Sequences | Length | Start Position | SEQ ID NO |
|---|---|---|---|---|
| Forward | 5′-aaaagtttcggtctggcttatc-3′ | 22 | 1354 | 356 |
| Probe | TET-5′-tttctctctgcatacgggctccagtg-3′-TAMRA | 26 | 1376 | 357 |
| Reverse | 5′-gaccaatggctggtaagtaatg-3′22 | 1412 | 358 | |

TABLE AAB
General_screening_panel_v1.4

| | Rel. Exp. (%) Ag3305, Run | | Rel. Exp. (%) Ag3305, Run |
|---|---|---|---|
| Tissue Name | 215648474 | Tissue Name | 215648474 |
| Adipose | 0.0 | Renal ca. TK-10 | 1.3 |
| Melanoma* Hs688(A).T | 0.0 | Bladder | 0.0 |
| Melanoma* Hs688(B).T | 0.0 | Gastric ca. (liver met.) NCI- | 0.0 |
| | | N87 | |
| Melanoma* M14 | 0.0 | Gastric ca. KATO III | 2.4 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell carcinoma | 0.0 | Colon ca.* (SW480 met) | 0.5 |
| SCC-4 | | SW620 | |
| Testis Pool | 100.0 | Colon ca. HT29 | 1.6 |
| Prostate ca.* (bone met) | 0.0 | Colon ca. HCT-116 | 4.8 |
| PC-3 | | | |
| Prostate Pool | 0.0 | Colon ca. CaCo-2 | 1.8 |
| Placenta | 0.0 | Colon cancer tissue | 2.8 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 4.4 | Colon ca. SW-48 | 0.0 |
| Ovarian ca. OVCAR-4 | 0.0 | Colon Pool | 1.4 |
| Ovarian ca. OVCAR-5 | 0.0 | Small Intestine Pool | 0.8 |
| Ovarian ca. IGROV-1 | 9.2 | Stomach Pool | 1.3 |
| Ovarian ca. OVCAR-8 | 0.0 | Bone Marrow Pool | 2.6 |
| Ovary | 0.5 | Fetal Heart | 0.0 |
| Breast ca. MCF-7 | 0.6 | Heart Pool | 2.8 |
| Breast ca. MDA-MB-231 | 2.3 | Lymph Node Pool | 1.8 |
| Breast ca. BT 549 | 0.9 | Fetal Skeletal Muscle | 0.0 |
| Breast ca. T47D | 1.2 | Skeletal Muscle Pool | 0.0 |
| Breast ca. MDA-N | 1.6 | Spleen Pool | 0.5 |
| Breast Pool | 2.8 | Thymus Pool | 1.8 |
| Trachea | 0.0 | CNS cancer (glio/astro) U87- | 0.0 |
| | | MG | |
| Lung | 0.0 | CNS cancer (glio/astro) U- | 1.1 |
| | | 118-MG | |
| Fetal Lung | 0.6 | CNS cancer (neuro; met) SK- | 0.0 |
| | | N-AS | |
| Lung ca. NCI-N417 | 0.5 | CNS cancer (astro) SF-539 | 0.0 |
| Lung ca. LX-1 | 1.8 | CNS cancer (astro) SNB-75 | 2.1 |
| Lung ca. NCI-H146 | 0.0 | CNS cancer (glio) SNB-19 | 2.5 |
| Lung ca. SHP-77 | 0.0 | CNS cancer (glio) SF-295 | 2.1 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 3.8 | Brain (fetal) | 0.0 |
| Lung ca. NCI-H460 | 0.0 | Brain (Hippocampus) Pool | 1.4 |
| Lung ca. HOP-62 | 0.0 | Cerebral Cortex Pool | 1.0 |
| Lung ca. NCI-H522 | 0.0 | Brain (Substantia nigra) Pool | 1.4 |
| Liver | 0.0 | Brain (Thalamus) Pool | 1.1 |
| Fetal Liver | 0.0 | Brain (whole) | 0.0 |
| Liver ca. HepG2 | 4.6 | Spinal Cord Pool | 0.0 |
| Kidney Pool | 13.3 | Adrenal Gland | 0.0 |
| Fetal Kidney | 0.6 | Pituitary gland Pool | 0.0 |
| Renal ca. 786-0 | 1.1 | Salivary Gland | 0.0 |
| Renal ca. A498 | 0.0 | Thyroid (female) | 0.0 |
| Renal ca. ACHN | 1.0 | Pancreatic ca. CAPAN2 | 0.0 |
| Renal ca. UO-31 | 0.0 | Pancreas Pool | 9.9 |

TABLE AAC
Panel 4D

| | Rel. Exp. (%) Ag3305, Run | | Rel. Exp. (%) Ag3305, Run |
|---|---|---|---|
| Tissue Name | 164682643 | Tissue Name | 164682643 |
| Secondary Th1 act | 11.7 | HUVEC IL-1beta | 4.2 |
| Secondary Th2 act | 12.2 | HUVEC IFN gamma | 29.9 |
| Secondary Tr1 act | 9.2 | HUVEC TNF alpha + IFN gamma | 6.4 |
| Secondary Th1 rest | 10.0 | HUVEC TNF alpha + IL4 | 7.3 |
| Secondary Th2 rest | 3.3 | HUVEC IL-11 | 7.9 |
| Secondary Tr1 rest | 27.5 | Lung Microvascular EC none | 19.6 |
| Primary Th1 act | 10.9 | Lung Microvascular EC | 32.8 |
| | | TNFalpha + IL-1beta | |
| Primary Th2 act | 14.6 | Microvascular Dermal EC none | 23.2 |
| Primary Tr1 act | 8.7 | Microsvasular Dermal EC | 13.3 |
| | | TNFalpha + IL-1beta | |
| Primary Th1 rest | 20.2 | Bronchial epithelium TNFalpha + | 12.0 |
| | | IL1beta | |
| Primary Th2 rest | 11.3 | Small airway epithelium none | 0.0 |
| Primary Tr1 rest | 17.6 | Small airway epithelium | 2.4 |
| | | TNFalpha + IL-1beta | |
| CD45RA CD4 lymphocyte | 5.1 | Coronery artery SMC rest | 0.0 |
| act | | | |
| CD45RO CD4 lymphocyte | 20.0 | Coronery artery SMC TNFalpha + | 0.0 |
| act | | IL-1beta | |
| CD8 lymphocyte act | 9.5 | Astrocytes rest | 1.7 |
| Secondary CD8 lymphocyte | 19.2 | Astrocytes TNFalpha + IL-1beta | 0.0 |
| rest | | | |
| Secondary CD8 lymphocyte | 2.4 | KU-812 (Basophil) rest | 24.7 |
| act | | | |
| CD4 lymphocyte none | 5.1 | KU-812 (Basophil) | 29.1 |
| | | PMA/ionomycin | |
| 2ry Th1/Th2/Tr1_anti-CD95 | 18.4 | CCD1106 (Keratinocytes) none | 18.9 |
| CH11 | | | |
| LAK cells rest | 24.7 | CCD1106 (Keratinocytes) | 3.4 |
| | | TNFalpha + IL-1beta | |
| LAK cells IL-2 | 24.3 | Liver cirrhosis | 3.0 |
| LAK cells IL-2 + IL-12 | 11.2 | Lupus kidney | 1.8 |
| LAK cells IL-2 + IFN gamma | 45.7 | NCI-H292 none | 18.6 |
| LAK cells IL-2 + IL-18 | 13.3 | NCI-H292 IL-4 | 34.4 |
| LAK cells PMA/ionomycin | 1.3 | NCI-H292 IL-9 | 2.5 |
| NK Cells IL-2 rest | 3.0 | NCI-H292 IL-13 | 1.3 |
| Two Way MLR 3 day | 27.9 | NCI-H292 IFN gamma | 0.8 |
| Two Way MLR 5 day | 8.0 | HPAEC none | 12.5 |
| Two Way MLR 7 day | 0.0 | HPAEC TNF alpha + IL-1 beta | 10.0 |
| PBMC rest | 11.7 | Lung fibroblast none | 5.4 |
| PBMC PWM | 19.1 | Lung fibroblast TNF alpha + IL-1 | 2.6 |
| | | beta | |
| PBMC PHA-L | 2.8 | Lung fibroblast IL-4 | 16.2 |
| Ramos (B cell) none | 73.2 | Lung fibroblast IL-9 | 4.1 |
| Ramos (B cell) ionomycin | 100.0 | Lung fibroblast IL-13 | 3.5 |
| B lymphocytes PWM | 6.6 | Lung fibroblast IFN gamma | 15.5 |
| B lymphocytes CD40L and | 44.8 | Dermal fibroblast CCD1070 rest | 35.1 |
| IL-4 | | | |
| EOL-1 dbcAMP | 4.2 | Dermal fibroblast CCD1070 TNF | 27.4 |
| | | alpha | |
| EOL-1 dbcAMP | 0.0 | Dermal fibroblast CCD1070 IL-1 | 1.7 |
| PMA/ionomycin | | beta | |
| Dendritic cells none | 3.7 | Dermal fibroblast IFN gamma | 3.0 |
| Dendritic cells LPS | 14.2 | Dermal fibroblast IL-4 | 3.3 |
| Dendritic cells anti-CD40 | 11.8 | IBD Colitis 2 | 0.5 |
| Monocytes rest | 0.0 | IBD Crohn's | 0.0 |
| Monocytes LPS | 8.1 | Colon | 1.2 |
| Macrophages rest | 21.3 | Lung | 0.0 |
| Macrophages LPS | 23.7 | Thymus | 5.9 |
| HUVEC none | 27.9 | Kidney | 18.6 |
| HUVEC starved | 39.2 | | |

TABLE AAD
Panel 5 Islet

| | Rel. Exp. (%) Ag3305, | | Rel. Exp. (%) Ag3305, |
|---|---|---|---|
| Tissue Name | Run 242322479 | Tissue Name | Run 242322479 |
| 97457_Patient-02go_adipose | 0.0 | 94709_Donor 2 AM - A_adipose | 13.0 |
| 97476_Patient-07sk_skeletal | 0.0 | 94710_Donor 2 AM - B_adipose | 0.0 |
| muscle | | | |
| 97477_Patient-07ut_uterus | 21.6 | 94711_Donor 2 AM - C_adipose | 100.0 |
| 97478_Patient-07pl_placenta | 0.0 | 94712_Donor 2 AD - A_adipose | 26.8 |
| 99167_Bayer Patient | 0.0 | 94713_Donor 2 AD - B_adipose | 0.0 |
| 97482_Patient-08ut_uterus | 0.0 | 94714_Donor 2 AD - C_adipose | 0.0 |
| 97483_Patient-08pl_placenta | 23.7 | 94742_Donor 3 U - A_Mesenchymal | 21.3 |
| | | Stem Cells | |
| 97486_Patient-09sk_skeletal | 0.0 | 94743_Donor 3 U - B_Mesenchymal | 0.0 |
| muscle | | Stem Cells | |
| 97487_Patient-09ut_uterus | 3.4 | 94730_Donor 3 AM - A_adipose | 42.0 |
| 97488_Patient-09pl_placenta | 7.9 | 94731_Donor 3 AM - B_adipose | 0.0 |
| 97492_Patient-10ut_uterus | 0.0 | 94732_Donor 3 AM - C_adipose | 17.4 |
| 97493_Patient-10pl_placenta | 0.0 | 94733_Donor 3 AD - A_adipose | 0.0 |
| 97495_Patient-11go_adipose | 11.7 | 94734_Donor 3 AD - B_adipose | 0.0 |
| 97496_Patient-11sk_skeletal | 8.5 | 94735_Donor 3 AD - C_adipose | 0.0 |
| muscle | | | |
| 97497_Patient-11ut_uterus | 5.2 | 77138_Liver_HepG2untreated | 84.1 |
| 97498_Patient-11pl_placenta | 0.0 | 73556_Heart_Cardiac stromal cells | 0.0 |
| | | (primary) | |
| 97500_Patient-12go_adipose | 17.9 | 81735_Small Intestine | 12.7 |
| 97501_Patient-12sk_skeletal | 15.1 | 72409_Kidney_Proximal Convoluted | 0.0 |
| muscle | | Tubule | |
| 97502_Patient-12ut_uterus | 8.0 | 82685_Small intestine_Duodenum | 0.0 |
| 97503_Patient-12pl_placenta | 0.0 | 90650_Adrenal_Adrenocortical | 0.0 |
| | | adenoma | |
| 94721_Donor 2 U | 0.0 | 72410_Kidney_HRCE | 12.9 |
| A_Mesenchymal Stem Cells | | | |
| 94722_Donor 2 U | 8.7 | 72411_Kidney_HRE | 59.5 |
| B_Mesenchymal Stem Cells | | | |
| 94723_Donor 2 U | 13.0 | 73139_Uterus_Uterine smooth muscle | 0.0 |
| C_Mesenchymal Stem Cells | | cells | |

CNS_neurodegeneration_v1.0 Summary: Ag3305 Results from one experiment with the CG57672-01 gene are not included because signal was high in the water-only control well (data not shown).

General_screening_panel_v1.4 Summary: Ag3305 Significant expression of the CG57672-01 gene is limited to testis (CT=32.2). Thus, expression of this gene could be used to distinguish testis from the other samples on this panel. Furthermore, therapeutic modulation of the activity of this gene or its protein product, using small molecule drugs, antibodies or protein therapeutics, may be of use in the treatment of reproductive disorders such as infertility.

Panel 4D Summary: Ag3305 The CG57672-01 gene is expressed at low levels in many of the samples on this panel, with highest expression in ionomycin-treated Ramos B cells (CT=31). This gene is expressed at low levels in members of the T-cell, B-cell, basophil, macrophage/dendritic cell, and peripheral blood mononuclear cell family, as well as in lung and dermal fibroblasts and microvascular endothelial cells. This ubiquitous pattern of expression suggests that this gene product may be involved in homeostatic processes for these and other cell types and tissues.

Therefore, therapeutic modulation of the activity of this gene or its protein product may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag3305 The CG57672-01 gene is expressed at low levels in several samples of adipocytes cultured in vitro from mesenchymal stem cells. Therefore, this gene product may be involved in adipocyte differentiation and its therapeutic modulation may be a treatment for obesity and obesity-related Type 2 diabetes.

NOV34

Expression of NOV34/CG57680-01 was assessed using the primer-probe set Ag3307, described in Table ABA. Results of the RTQ-PCR runs are shown in Tables ABB, ABC and ABD.

TABLE ABA
Probe Name Ag3307

| Primers | Sequences | Length | Start Position | SEQ ID NO |
|---|---|---|---|---|
| Forward | 5′-ccagaacctacaagggaacaat-3′ | 22 | 582 | 359 |
| Probe | TET-5′-cacaaatgaagaaattgatcatttcaaacg-3′-TAMRA | 30 | 623 | 360 |
| Reverse | 5′-ttctacttcttcggctgacctt-3′ | 22 | 653 | 361 |

TABLE ABB
CNS_neurodegeneration_v1.0

| | Rel. Exp. (%) Ag3307, Run | | Rel. Exp. (%) Ag3307, Run |
|---|---|---|---|
| Tissue Name | 210138283 | Tissue Name | 210138283 |
| AD 1 Hippo | 11.6 | Control (Path) 3 Temporal | 23.2 |
| | | Ctx | |
| AD 2 Hippo | 10.4 | Control (Path) 4 Temporal | 36.6 |
| | | Ctx | |
| AD 3 Hippo | 13.8 | AD 1 Occipital Ctx | 44.4 |
| AD 4 Hippo | 7.9 | AD 2 Occipital Ctx | 0.0 |
| | | (Missing) | |
| AD 5 Hippo | 100.0 | AD 3 Occipital Ctx | 30.8 |
| AD 6 Hippo | 18.7 | AD 4 Occipital Ctx | 12.5 |
| Control 2 Hippo | 9.6 | AD 5 Occipital Ctx | 34.6 |
| Control 4 Hippo | 11.6 | AD 6 Occipital Ctx | 13.7 |
| Control (Path) 3 Hippo | 15.7 | Control 1 Occipital Ctx | 10.0 |
| AD 1 Temporal Ctx | 22.4 | Control 2 Occipital Ctx | 14.0 |
| AD 2 Temporal Ctx | 23.8 | Control 3 Occipital Ctx | 40.6 |
| AD 3 Temporal Ctx | 19.5 | Control 4 Occipital Ctx | 13.0 |
| AD 4 Temporal Ctx | 19.5 | Control (Path) 1 Occipital | 36.1 |
| | | Ctx | |
| AD 5 Inf Temporal Ctx | 85.3 | Control (Path) 2 Occipital | 36.3 |
| | | Ctx | |
| AD 5 Sup Temporal Ctx | 34.6 | Control (Path) 3 Occipital | 11.7 |
| | | Ctx | |
| AD 6 Inf Temporal Ctx | 36.1 | Control (Path) 4 Occipital | 52.9 |
| | | Ctx | |
| AD 6 Sup Temporal Ctx | 44.1 | Control 1 Parietal Ctx | 17.2 |
| Control 1 Temporal Ctx | 16.5 | Control 2 Parietal Ctx | 63.7 |
| Control 2 Temporal Ctx | 13.1 | Control 3 Parietal Ctx | 22.4 |
| Control 3 Temporal Ctx | 18.3 | Control (Path) 1 Parietal | 32.5 |
| | | Ctx | |
| Control 4 Temporal Ctx | 22.1 | Control (Path) 2 Parietal | 28.5 |
| | | Ctx | |
| Control (Path) 1 Temporal | 29.7 | Control (Path) 3 Parietal | 24.5 |
| Ctx | | Ctx | |
| Control (Path) 2 Temporal | 25.9 | Control (Path) 4 Parietal | 49.7 |
| Ctx | | Ctx | |

TABLE ABC
General_screening_panel_v1.4

| | Rel. Exp. (%) Ag3307, Run | | Rel. Exp. (%) Ag3307, Run |
|---|---|---|---|
| Tissue Name | 215648237 | Tissue Name | 215648237 |
| Adipose | 1.1 | Renal ca. TK-10 | 1.8 |
| Melanoma* Hs688(A).T | 0.2 | Bladder | 4.0 |
| Melanoma* Hs688(B).T | 0.5 | Gastric ca. (liver met.) NCI- | 1.4 |
| | | N87 | |
| Melanoma* MI4 | 0.6 | Gastric ca. KATO III | 1.6 |
| Melanoma* LOXIMVI | 0.6 | Colon ca. SW-948 | 0.5 |
| Melanoma* SK-MEL-5 | 1.4 | Colon ca. SW480 | 1.9 |
| Squamous cell carcinoma | 0.9 | Colon ca.* (SW480 met) | 1.3 |
| SCC-4 | | SW620 | |
| Testis Pool | 1.8 | Colon ca. HT29 | 1.3 |
| Prostate ca.* (bone met) | 1.2 | Colon ca. HCT-116 | 1.3 |
| PC-3 | | | |
| Prostate Pool | 2.2 | Colon ca. CaCo-2 | 2.7 |
| Placenta | 3.3 | Colon cancer tissue | 1.8 |
| Uterus Pool | 0.4 | Colon ca. SW1116 | 1.8 |
| Ovarian ca. OVCAR-3 | 1.0 | Colon ca. Colo-205 | 0.0 |
| Ovarian ca. SK-OV-3 | 0.9 | Colon ca. SW-48 | 0.4 |
| Ovarian ca. OVCAR-4 | 0.1 | Colon Pool | 100.0 |
| Ovarian ca. OVCAR-5 | 4.2 | Small Intestine Pool | 3.0 |
| Ovarian ca. IGROV-1 | 5.2 | Stomach Pool | 4.3 |
| Ovarian ca. OVCAR-8 | 1.1 | Bone Marrow Pool | 1.8 |
| Ovary | 1.2 | Fetal Mean | 3.5 |
| Breast ca. MCF-7 | 3.4 | Heart Pool | 1.1 |
| Breast ca. MDA-MB-231 | 3.2 | Lymph Node Pool | 2.9 |
| Breast ca. BT 549 | 3.6 | Fetal Skeletal Muscle | 2.6 |
| Breast ca. T47D | 7.8 | Skeletal Muscle Pool | 1.3 |
| Breast ca. MDA-N | 0.9 | Spleen Pool | 13.8 |
| Breast Pool | 4.2 | Thymus Pool | 16.7 |
| Trachea | 3.9 | CNS cancer (glio/astro) U87- | 2.0 |
| | | MG | |
| Lung | 3.5 | CNS cancer (glio/astro) U- | 6.4 |
| | | 118-MG | |
| Fetal Lung | 40.9 | CNS cancer (neuro; met) SK- | 3.7 |
| | | N-AS | |
| Lung ca. NCI-N417 | 3.2 | CNS cancer (astro) SF-539 | 0.3 |
| Lung ca. LX-1 | 1.1 | CNS cancer (astro) SNB-75 | 2.5 |
| Lung ca. NCI-H146 | 1.5 | CNS cancer (glio) SNB-19 | 4.0 |
| Lung ca. SHP-77 | 4.8 | CNS cancer (glio) SF-295 | 3.2 |
| Lung ca. A549 | 0.0 | Brain (Amygdala) Pool | 1.8 |
| Lung ca. NCI-H526 | 0.6 | Brain (cerebellum) | 2.8 |
| Lung ca. NCI-H23 | 1.1 | Brain (fetal) | 5.2 |
| Lung ca. NCI-H460 | 5.3 | Brain (Hippocampus) Pool | 2.1 |
| Lung ca. HOP-62 | 1.6 | Cerebral Cortex Pool | 2.7 |
| Lung ca. NCI-H522 | 1.2 | Brain (Substantia nigra) Pool | 2.3 |
| Liver | 0.0 | Brain (Thalamus) Pool | 4.2 |
| Fetal Liver | 1.1 | Brain (whole) | 1.1 |
| Liver ca. HepG2 | 0.1 | Spinal Cord Pool | 1.7 |
| Kidney Pool | 4.4 | Adrenal Gland | 0.7 |
| Fetal Kidney | 9.8 | Pituitary gland Pool | 0.8 |
| Renal ca. 786-0 | 3.0 | Salivary Gland | 0.5 |
| Renal ca. A498 | 1.2 | Thyroid (female) | 1.8 |
| Renal ca. ACHN | 0.8 | Pancreatic ca. CAPAN2 | 2.6 |
| Renal ca. UO-31 | 0.3 | Pancreas Pool | 3.4 |

TABLE ABD
Panel 4D

| | Rel. Exp. (%) Ag3307, Run | | Rel. Exp. (%) Ag3307, Run |
|---|---|---|---|
| Tissue Name | 164335286 | Tissue Name | 164335286 |
| Secondary Th1 act | 3.1 | HUVEC IL-1beta | 0.8 |
| Secondary Th2 act | 4.7 | HUVEC IFN gamma | 3.5 |
| Secondary Tr1 act | 6.1 | HUVEC TNF alpha + IFN gamma | 2.2 |
| Secondary Th1 rest | 6.7 | HUVEC TNF alpha + IL4 | 0.6 |
| Secondary Th2 rest | 5.0 | HUVEC IL-11 | 0.8 |
| Secondary Tr1 rest | 7.4 | Lung Microvascular EC none | 0.4 |
| Primary Th1 act | 4.6 | Lung Microvascular EC | 0.2 |
| | | TNFalpha + IL-1beta | |
| Primary Th2 act | 6.0 | Microvascular Dermal EC none | 0.7 |
| Primary Tr1 act | 9.9 | Microsvasular Dermal EC | 0.0 |
| | | TNFalpha + IL-1beta | |
| Primary Th1 rest | 37.6 | Bronchial epithelium TNFalpha + | 1.3 |
| | | IL1beta | |
| Primary Th2 rest | 24.5 | Small airway epithelium none | 0.7 |
| Primary Tr1 rest | 13.5 | Small airway epithelium | 3.3 |
| | | TNFalpha + IL-1beta | |
| CD45RA CD4 lymphocyte | 2.5 | Coronery artery SMC rest | 0.3 |
| act | | | |
| CD45RO CD4 lymphocyte | 8.6 | Coronery artery SMC TNFalpha + | 0.4 |
| act | | IL-1beta | |
| CD8 lymphocyte act | 4.6 | Astrocytes rest | 1.1 |
| Secondary CD8 lymphocyte | 3.3 | Astrocytes TNFalpha + IL-1 beta | 1.8 |
| rest | | | |
| Secondary CD8 lymphocyte | 8.0 | KU-812 (Basophil) rest | 3.8 |
| act | | | |
| CD4 lymphocyte none | 6.3 | KU-812 (Basophil) | 8.7 |
| | | PMA/ionomycin | |
| 2ry Th1/Th2/Tr1_anti-CD95 | 14.9 | CCD1106 (Keratinocytes) none | 1.2 |
| CH11 | | | |
| LAK cells rest | 5.6 | CCD1106 (Keratinocytes) | 0.0 |
| | | TNFalpha + IL-1 beta | |
| LAK cells IL-2 | 20.4 | Liver cirrhosis | 1.8 |
| LAK cells IL-2 + IL-12 | 6.0 | Lupus kidney | 1.5 |
| LAK cells IL-2 + IFN gamma | 9.8 | NCI-H292 none | 5.2 |
| LAK cells IL-2 + IL-18 | 10.3 | NCI-H292 IL-4 | 7.8 |
| LAK cells PMA/ionomycin | 1.7 | NCI-H292 IL-9 | 18.2 |
| NK Cells IL-2 rest | 14.2 | NCI-H292 IL-13 | 7.0 |
| Two Way MLR 3 day | 14.9 | NCI-H292 IFN gamma | 8.5 |
| Two Way MLR 5 day | 3.0 | HPAEC none | 1.7 |
| Two Way MLR 7 day | 8.1 | HPAEC TNF alpha + IL-1 beta | 0.3 |
| PBMC rest | 5.1 | Lung fibroblast none | 2.6 |
| PBMC PWM | 13.8 | Lung fibroblast TNF alpha + IL-1 | 2.2 |
| | | beta | |
| PBMC PHA-L | 6.7 | Lung fibroblast IL-4 | 4.0 |
| Ramos (B cell) none | 10.7 | Lung fibroblast IL-9 | 2.8 |
| Ramos (B cell) ionomycin | 24.0 | Lung fibroblast IL-13 | 2.5 |
| B lymphocytes PWM | 24.1 | Lung fibroblast IFN gamma | 3.7 |
| B lymphocytes CD40L and | 54.3 | Dermal fibroblast CCD1070 rest | 3.5 |
| IL-4 | | | |
| EOL-1 dbcAMP | 1.7 | Dermal fibroblast CCD1070 TNF | 18.8 |
| | | alpha | |
| EOL-1 dbcAMP | 1.8 | Dermal fibroblast CCD1070 IL-1 | 0.8 |
| PMA/ionomycin | | beta | |
| Dendritic cells none | 1.4 | Dermal fibroblast IFN gamma | 1.4 |
| Dendritic cells LPS | 0.4 | Dermal fibroblast IL-4 | 1.0 |
| Dendritic cells anti-CD40 | 0.5 | IBD Colitis 2 | 3.3 |
| Monocytes rest | 1.1 | IBD Crohn's | 2.2 |
| Monocytes LPS | 0.7 | Colon | 7.2 |
| Macrophages rest | 2.8 | Lung | 1.6 |
| Macrophages LPS | 0.3 | Thymus | 8.8 |
| HUVEC none | 1.7 | Kidney | 100.0 |
| HUVEC starved | 4.5 | | |

CNS_neurodegeneration_v1.0 Summary: Ag3307 This panel confirms the expression of this gene at low to moderate levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3307 The CG57680-01 gene is expressed at low levels in many of the tissues on this panel, with the highest expression in a normal colon sample (CT=28.8). Significant expression of this gene is also detected in fetal lung (CT=30.1). Interestingly, this gene is expressed at much lower levels in adult lung (CT=33.6), suggesting that expression of this gene can be used to distinguish fetal lung from adult lung.

This gene is also expressed at low levels in many regions of the central nervous system, including amygdala, substantia nigra, thalamus, hippocampus, cerebellum, cerebral cortex, and spinal cord. The CG57680-01 gene encodes a protein with homology to cyclophilin-type peptidyl-prolyl cis-trans isomerase. Cyclophilin is a specific high-affinity binding protein for the immunosuppressant agent cyclosporin A. Neuroimmunophilin ligands, such as cyclosporin A, are a class of compounds that hold great promise for the treatment of nerve injuries and neurological disease. Therefore, modulation of the activity of the CG57680-01 gene or its protein product may be of use in the treatment of neurodegenerative disorders such as Alzheimer's disease and Parkinson's disease.

Among tissues with metabolic or endocrine function, this gene is expressed at low levels in thyroid, pancreas, fetal heart, and fetal skeletal muscle. Therefore, therapeutic modulation of the activity of this gene may prove useful in the treatment of endocrine/metabolically related diseases, such as obesity and diabetes.

References:

1. Gold B G. Neuroimmunophilin ligands: evaluation of their therapeutic potential for the treatment of neurological disorders. Expert Opin Investig Drugs 2000 October;9(10):2331-42

Neuroimmunophilin ligands are a class of compounds that hold great promise for the treatment of nerve injuries and neurological disease. In contrast to neurotrophins (e.g., nerve growth factor), these compounds readily cross the blood-brain barrier, being orally effective in a variety of animal models of ischaemia, traumatic nerve injury and human neurodegenerative disorders. A further distinction is that neuroimmunophilin ligands act via unique receptors that are unrelated to the classical neurotrophic receptors e.g., trk), making it unlikely that clinical trials will encounter the same difficulties found with the neurotrophins. Another advantage is that two neuroimmunophilin ligands (cyclosporin A and FK-506) have already been used in humans (as immunosuppressant drugs). Whereas both cyclosporin A and FK-506 demonstrate neuroprotective actions, only FK-506 and its derivatives have been clearly shown to exhibit significant neuroregenerative activity. Accordingly, the neuroprotective and neuroregenerative properties seem to arise via different mechanisms. Furthermore, the neuroregenerative property does not involve calcineurin inhibition (essential for immunosuppression). This is important since most of the limiting side effects produced by these drugs arise via calcineurin inhibition. A major breakthrough for the development of this class of compounds for the treatment of human neurological disorders was the ability to separate the neuroregenerative property of FK-506 from its immunosuppressant action via the development of non-immunosuppressant (non-calcineurin inhibiting) derivatives. Further studies revealed that different receptor subtypes, or FK-506-binding proteins (FKBPs), mediate immunosuppression and nerve regeneration (FKBP-12 and FKBP-52, respectively, the latter being a component of steroid receptor complexes). Thus, steroid receptor chaperone proteins represent novel targets for future drug development of novel classes of compounds for the treatment of a variety of human neurological disorders, including traumatic injury (e.g., peripheral nerve and spinal cord), chemical exposure (e.g., vinca alkaloids, Taxol) and neurodegenerative disease (e.g., diabetic neuropathy and Parkinson's disease).

PMID: 11060810

Panel 4D Summary: Ag3307 Expression of the CG57680-01 gene is highest in kidney (CT=29.2). This gene is also expressed at moderate to low levels in numerous cell types, but is expressed at higher levels (CTs 20-32) in T cells and B cells. Therefore, small molecule antagonists that block the function of the CG57680-01 protein may be useful to reduce or eliminate the symptoms of Crohn's disease, ulcerative colitis, multiple sclerosis, chronic obstructive pulmonary disease, asthma, emphysema, rheumatoid arthritis, lupus erythematosus, or psoriasis.

NOV35

Expression of NOV35/CG57670-01 was assessed using the primer-probe set Ag3304, described in Table ACA. Results of the RTQ-PCR runs are shown in Tables ACB, ACC, ACD, ACE and ACF.

TABLE ACA
Probe Name Ag3304

| Primers | Sequences | Length | Start Position | SEQ ID NO |
|---|---|---|---|---|
| Forward | 5′-aaagctttgcttctgactccat-3′ | 22 | 297 | 362 |
| Probe | TET-5′-ctctaccagcccattgctgtggct-3′-TAMRA | 24 | 320 | 363 |
| Reverse | 5′-ttggatctcaggtcctttagt-3′ | 22 | 350 | 364 |

TABLE ACB
CNS_neurodegeneration_v1.0

| | Rel. Exp. (%) Ag3304, Run | | Rel. Exp. (%) Ag3304, Run |
|---|---|---|---|
| Tissue Name | 210063833 | Tissue Name | 210063833 |
| AD 1 Hippo | 2.0 | Control (Path) 3 Temporal | 1.2 |
| | | Ctx | |
| AD 2 Hippo | 22.2 | Control (Path) 4 Temporal | 14.9 |
| | | Ctx | |
| AD 3 Hippo | 3.2 | AD 1 Occipital Ctx | 17.3 |
| AD 4 Hippo | 24.1 | AD 2 Occipital Ctx | 0.0 |
| | | (Missing) | |
| AD 5 Hippo | 45.4 | AD 3 Occipital Ctx | 7.5 |
| AD 6 Hippo | 70.2 | AD 4 Occipital Ctx | 48.3 |
| Control 2 Hippo | 51.8 | AD 5 Occipital Ctx | 9.0 |
| Control 4 Hippo | 18.2 | AD 6 Occipital Ctx | 15.7 |
| Control (Path) 3 Hippo | 14.8 | Control 1 Occipital Ctx | 3.1 |
| AD 1 Temporal Ctx | 8.0 | Control 2 Occipital Ctx | 50.0 |
| AD 2 Temporal Ctx | 85.3 | Control 3 Occipital Ctx | 24.0 |
| AD 3 Temporal Ctx | 4.1 | Control 4 Occipital Ctx | 11.7 |
| AD 4 Temporal Ctx | 23.3 | Control (Path) 1 Occipital | 57.0 |
| | | Ctx | |
| AD 5 Inf Temporal Ctx | 11.3 | Control (Path) 2 Occipital | 21.5 |
| | | Ctx | |
| AD 5 Sup Temporal Ctx | 49.7 | Control (Path) 3 Occipital | 5.5 |
| | | Ctx | |
| AD 6 Inf Temporal Ctx | 31.6 | Control (Path) 4 Occipital | 12.2 |
| | | Ctx | |
| AD 6 Sup Temporal Ctx | 29.3 | Control 1 Parietal Ctx | 17.2 |
| Control 1 Temporal Ctx | 16.6 | Control 2 Parietal Ctx | 13.8 |
| Control 2 Temporal Ctx | 46.0 | Control 3 Parietal Ctx | 16.3 |
| Control 3 Temporal Ctx | 41.8 | Control (Path) 1 Parietal | 70.2 |
| | | Ctx | |
| Control 3 Temporal Ctx | 3.6 | Control (Path) 2 Parietal | 21.3 |
| | | Ctx | |
| Control (Path) 1 Temporal | 100.0 | Control (Path) 3 Parietal | 5.4 |
| Ctx | | Ctx | |
| Control (Path) 2 Temporal | 89.5 | Control (Path) 4 Parietal | 15.0 |
| Ctx | | Ctx | |

TABLE ACC
General_screening_panel_v1.4

| | Rel. Exp. (%) Ag3304, Run | | Rel. Exp. (%) Ag3304, Run |
|---|---|---|---|
| Tissue Name | 215648468 | Tissue Name | 215648468 |
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI- | 0.0 |
| | | N87 | |
| Melanoma* M14 | 0.0 | Gastric ca. KATO III | 0.0 |
| Melanoma* LOXIMVI | 0.0 | Colon ca SW-948 | 100.0 |
| Melanoma* SK-MEL-5 | 0.0 | Colon ca. SW480 | 0.0 |
| Squamous cell carcinoma | 0.0 | Colon ca.* (SW480 met) | 0.0 |
| SCC-4 | | SW620 | |
| Testis Pool | 0.0 | Colon ca. HT29 | 0.0 |
| Prostate ca.* (bone met) | 0.0 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer (glio/astro) U87- | 0.0 |
| | | MG | |
| Lung | 0.0 | CNS cancer (glio/astro) U- | 0.0 |
| | | 118-MG | |
| Fetal Lung | 0.0 | CNS cancer (neuro; met) SK- | 0.0 |
| | | N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) Pool | 0.0 |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

TABLE ACD
General_screening_panel_v1.5

| | Rel. Exp. (%) | Rel. Exp. (%) | | Rel. Exp. (%) | Rel. Exp. (%) |
|---|---|---|---|---|---|
| | Ag3304, Run | Ag3304, Run | | Ag3304, Run | Ag3304, Run |
| Tissue Name | 244373099 | 248445829 | Tissue Name | 244373099 | 248445829 |
| Adipose | 0.5 | 5.6 | Renal ca. TK-10 | 8.1 | 6.2 |
| Melanoma* | 15.0 | 0.0 | Bladder | 4.6 | 35.6 |
| Hs688(A).T | | | | | |
| Melanoma* | 19.9 | 1.0 | Gastric ca. (liver | 3.2 | 11.4 |
| Hs688(B).T | | | met.) NCI-N87 | | |
| Melanoma* M14 | 17.3 | 0.5 | Gastric ca. KATO III | 3.4 | 18.4 |
| Melanoma* | 13.7 | 0.7 | Colon ca. SW-948 | 0.6 | 39.8 |
| LOXIMVI | | | | | |
| Melanoma* SK- | 11.0 | 0.0 | Colon ca. SW480 | 7.6 | 6.2 |
| MEL-5 | | | | | |
| Squamous cell | 15.6 | 47.3 | Colon ca.* (SW480 | 4.1 | 5.3 |
| carcinoma SCC-4 | | | met) SW620 | | |
| Testis Pool | 1.1 | 4.9 | Colon ca. HT29 | 6.3 | 3.8 |
| Prostate ca.* | 11.6 | 19.3 | Colon ca. HCT-116 | 2.6 | 5.8 |
| (bone met) PC-3 | | | | | |
| Prostate Pool | 3.8 | 1.7 | Colon ca. CaCo-2 | 0.7 | 4.7 |
| Placenta | 0.0 | 5.1 | Colon cancer tissue | 0.0 | 11.2 |
| Uterus Pool | 0.3 | 1.9 | Colon ca. SW1116 | 7.8 | 2.1 |
| Ovarian ca. | 0.0 | 2.1 | Colon ca. Colo-205 | 4.0 | 39.8 |
| OVCAR-3 | | | | | |
| Ovarian ca. SK- | 1.1 | 13.7 | Colon ca. SW-48 | 4.4 | 4.3 |
| OV-3 | | | | | |
| Ovarian ca. | 5.0 | 100.0 | Colon Pool | 0.0 | 1.3 |
| OVCAR-4 | | | | | |
| Ovarian ca. | 6.4 | 43.2 | Small Intestine Pool | 0.7 | 4.4 |
| OVCAR-5 | | | | | |
| Ovarian ca. | 1.1 | 26.6 | Stomach Pool | 1.0 | 22.4 |
| IGROV-1 | | | | | |
| Ovarian ca. | 11.3 | 5.8 | Bone Marrow Pool | 3.7 | 5.4 |
| OVCAR-8 | | | | | |
| Ovary | 0.3 | 13.7 | Fetal Heart | 0.0 | 12.0 |
| Breast ca. MCF-7 | 3.8 | 3.6 | Heart Pool | 0.3 | 12.4 |
| Breast ca. MDA- | 1.3 | 3.3 | Lymph Node Pool | 0.2 | 5.6 |
| MB-231 | | | | | |
| Breast ca. BT 549 | 3.6 | 0.9 | Fetal Skeletal Muscle | 0.8 | 1.4 |
| Breast ca. T47D | 0.3 | 0.9 | Skeletal Muscle Pool | 0.9 | 3.1 |
| Breast ca. MDA-N | 0.2 | 3.0 | Spleen Pool | 2.2 | 12.2 |
| Breast Pool | 0.0 | 6.6 | Thymus Pool | 2.1 | 7.3 |
| Trachea | 0.0 | 5.4 | CNS cancer | 0.8 | 1.3 |
| | | | (glio/astro) U87-MG | | |
| Lung | 0.0 | 7.9 | CNS cancer | 2.7 | 22.7 |
| | | | (glio/astro) U-118-MG | | |
| Fetal Lung | 0.4 | 13.9 | CNS cancer | 3.4 | 1.2 |
| | | | (neuro;met) SK-N- | | |
| | | | AS | | |
| Lung ca. NCI- | 2.1 | 0.9 | CNS cancer (astro) | 2.8 | 4.3 |
| N417 | | | SF-539 | | |
| Lung ca. LX-1 | 24.3 | 9.7 | CNS cancer (astro) | 5.0 | 15.4 |
| | | | SNB-75 | | |
| Lung ca. NCI- | 0.7 | 0.0 | CNS cancer (glio) | 1.4 | 28.5 |
| H146 | | | SNB-19 | | |
| Lung ca. SHP-77 | 5.1 | 12.1 | CNS cancer (glio) | 16.2 | 45.1 |
| | | | SF-295 | | |
| Lung ca. A549 | 19.9 | 3.9 | Brain (Amygdala) | 4.3 | 8.4 |
| | | | Pool | | |
| Lung ca. NCI- | 2.1 | 0.7 | Brain (cerebellum) | 3.2 | 3.1 |
| H526 | | | | | |
| Lung ca. NCI-H23 | 29.5 | 63.7 | Brain (fetal) | 1.3 | 5.6 |
| Lung ca. NCI- | 100.0 | 18.4 | Brain (Hippocampus) | 0.5 | 2.8 |
| H460 | | | Pool | | |
| Lung ca. HOP-62 | 16.7 | 4.1 | Cerebral Cortex Pool | 0.9 | 3.1 |
| Lung ca. NCI- | 3.0 | 1.6 | Brain (Substantia | 4.1 | 31.0 |
| H522 | | | nigra) Pool | | |
| Liver | 0.4 | 2.3 | Brain (Thalamus) | 3.3 | 5.4 |
| | | | Pool | | |
| Fetal Liver | 0.0 | 5.0 | Brain (whole) | 0.0 | 17.0 |
| Liver ca. HepG2 | 3.3 | 4.8 | Spinal Cord Pool | 0.3 | 2.7 |
| Kidney Pool | 1.4 | 4.0 | Adrenal Gland | 15.7 | 4.6 |
| Fetal Kidney | 1.2 | 4.2 | Pituitary gland Pool | 0.0 | 36.9 |
| Renal ca. 786-0 | 20.0 | 9.2 | Salivary Gland | 0.0 | 0.0 |
| Renal ca. A498 | 3.4 | 16.5 | Thyroid (female) | 0.3 | 8.2 |
| Renal ca. ACHN | 6.3 | 8.7 | Pancreatic ca. | 11.1 | 26.2 |
| | | | CAPAN2 | | |
| Renal ca. UO-31 | 7.6 | 6.3 | Pancreas Pool | 1.9 | 19.9 |

TABLE ACE
Panel 4D

| | Rel. Exp. (%) Ag3304, Run | | Rel. Exp. (%) Ag3304, Run |
|---|---|---|---|
| Tissue Name | 164682642 | Tissue Name | 164682642 |
| Secondary Th1 act | 9.4 | HUVEC IL-1beta | 8.6 |
| Secondary Th2 act | 9.3 | HUVEC IFN gamma | 11.7 |
| Secondary Tr1 act | 20.9 | HUVEC TNF alpha + IFN gamma | 27.0 |
| Secondary Th1 rest | 3.5 | HUVEC TNF alpha + IL4 | 14.4 |
| Secondary Th2 rest | 2.1 | HUVEC IL-11 | 15.0 |
| Secondary Tr1 rest | 1.5 | Lung Microvascular EC none | 8.0 |
| Primary Th1 act | 10.1 | Lung Microvascular EC | 13.3 |
| | | TNFalpha + IL-1beta | |
| Primary Th2 act | 8.7 | Microvascular Dermal EC none | 6.1 |
| Primary Tr1 act | 25.0 | Microsvasular Dermal EC | 15.2 |
| | | TNFalpha + IL-1beta | |
| Primary Th1 rest | 25.9 | Bronchial epithelium TNFalpha + | 54.3 |
| | | IL1beta | |
| Primary Th2 rest | 8.9 | Small airway epithelium none | 20.4 |
| Primary Tr1 rest | 5.4 | Small airway epithelium | 51.4 |
| | | TNFalpha + IL-1beta | |
| CD45RA CD4 lymphocyte | 5.4 | Coronery artery SMC rest | 30.1 |
| act | | | |
| CD45RO CD4 lymphocyte | 25.2 | Coronery artery SMC TNFalpha + | 10.2 |
| act | | IL-1beta | |
| CD8 lymphocyte act | 43.2 | Astrocytes rest | 17.7 |
| Secondary CD8 lymphocyte | 31.0 | Astrocytes TNFalpha + IL-1beta | 25.7 |
| rest | | | |
| Secondary CD8 lymphocyte | 13.6 | KU-812 (Basophil) rest | 4.0 |
| act | | | |
| CD4 lymphocyte none | 1.6 | KU-812 (Basophil) | 20.3 |
| | | PMA/ionomycin | |
| 2ry Th1/Th2/Tr1_anti-CD95 | 6.2 | CCD1106 (Keratinocytes) none | 21.0 |
| CH11 | | | |
| LAK cells rest | 5.3 | CCD1106 (Keratinocytes) | 58.6 |
| | | TNFalpha + IL-1beta | |
| LAK cells IL-2 | 11.9 | Liver cirrhosis | 2.7 |
| LAK cells IL-2 + IL-12 | 11.5 | Lupus kidney | 3.7 |
| LAK cells IL-2 + IFN gamma | 11.2 | NCI-H292 none | 31.2 |
| LAK cells IL-2 + IL-18 | 17.9 | NCI-H292 IL-4 | 58.2 |
| LAK cells PMA/ionomycin | 18.3 | NCI-H292 IL-9 | 100.0 |
| NK Cells IL-2 rest | 17.1 | NCI-H292 IL-13 | 1.4 |
| Two Way MLR 3 day | 12.1 | NCI-H292 IFN gamma | 35.6 |
| Two Way MLR 5 day | 6.4 | HPAEC none | 12.5 |
| Two Way MLR 7 day | 10.7 | HPAEC TNF alpha + IL-1 beta | 21.0 |
| PBMC rest | 3.3 | Lung fibroblast none | 21.0 |
| PBMC PWM | 33.9 | Lung fibroblast TNF alpha + IL-1 | 11.6 |
| | | beta | |
| PBMC PHA-L | 33.9 | Lung fibroblast IL-4 | 18.8 |
| Ramos (B cell) none | 8.0 | Lung fibroblast IL-9 | 26.4 |
| Ramos (B cell) ionomycin | 32.5 | Lung fibroblast IL-13 | 20.9 |
| B lymphocytes PWM | 36.9 | Lung fibroblast IFN gamma | 67.4 |
| B lymphocytes CD40L and | 11.3 | Dermal fibroblast CCD1070 rest | 62.0 |
| IL-4 | | | |
| EOL-1 dbcAMP | 1.9 | Dermal fibroblast CCD1070 TNF | 94.6 |
| | | alpha | |
| EOL-1 dbcAMP | 23.0 | Dermal fibroblast CCD1070 IL-1 | 16.7 |
| PMA/ionomycin | | beta | |
| Dendritic cells none | 15.4 | Dermal fibroblast IFN gamma | 16.4 |
| Dendritic cells LPS | 34.9 | Dermal fibroblast IL-4 | 43.5 |
| Dendritic cells anti-CD40 | 9.5 | IBD Colitis 2 | 0.3 |
| Monocytes rest | 16.4 | IBD Crohn's | 0.8 |
| Monocytes LPS | 12.9 | Colon | 12.1 |
| Macrophages rest | 30.8 | Lung | 9.0 |
| Macrophages LPS | 29.5 | Thymus | 0.7 |
| HUVEC none | 13.5 | Kidney | 10.2 |
| HUVEC starved | 38.4 | | |

TABLE ACF
Panel 5 Islet

| | Rel. Exp. (%) Ag3304, | | Rel. Exp. (%) Ag3304, |
|---|---|---|---|
| Tissue Name | Run 242322478 | Tissue Name | Run 242322478 |
| 97457_Patient-02go_adipose | 9.0 | 94709_Donor 2 AM - A_adipose | 12.1 |
| 97476_Patient-07sk_skeletal | 11.2 | 94710_Donor 2 AM - B_adipose | 6.1 |
| muscle | | | |
| 97477_Patient-07ut_uterus | 20.0 | 94711_Donor 2 AM - C_adipose | 2.4 |
| 97478_Patient-07pl_placenta | 8.1 | 94712_Donor 2 AD - A_adipose | 19.6 |
| 99167_Bayer Patient 1 | 42.3 | 94713_Donor 2 AD - B_adipose | 16.8 |
| 97482_Patient-08ut_uterus | 8.8 | 94714_Donor 2 AD - C_adipose | 15.8 |
| 97483_Patient-08pl_placenta | 4.6 | 94742_Donor 3 U - A Mesenchymal | 4.9 |
| | | Stem Cells | |
| 97486_Patient-09sk_skeletal | 6.2 | 94743_Donor 3 U - B_Mesenchymal | 6.4 |
| muscle | | Stem Cells | |
| 97487_Patient-09ut_uterus | 12.4 | 94730_Donor 3 AM - A_adipose | 10.8 |
| 97488_Patient-09pl_placenta | 3.9 | 94731_Donor 3 AM - B_adipose | 8.0 |
| 97492_Patient-10ut_uterus | 8.0 | 94732_Donor 3 AM - C_adipose | 6.2 |
| 97493_Patient-10pl_placenta | 3.4 | 94733_Donor 3 AD - A_adipose | 32.8 |
| 97495_Patient-11go_adipose | 2.0 | 94734_Donor 3 AD - B_adipose | 11.7 |
| 97496_Patient-11sk_skeletal | 4.7 | 94735_Donor 3 AD - C_adipose | 23.5 |
| muscle | | | |
| 97497_Patient-11ut_uterus | 2.9 | 77138_Liver_HepG2untreated | 9.2 |
| 97498_Patient-11pl_placenta | 1.8 | 73556_Heart_Cardiac stromal cells | 2.4 |
| | | (primary) | |
| 97500_Patient-12go_adipose | 4.2 | 81735_Small Intestine | 3.8 |
| 97501_Patient-12sk_skeletal | 14.1 | 72409_Kidney_Proximal Convoluted | 16.8 |
| muscle | | Tubule | |
| 97502_Patient-12ut_uterus | 3.5 | 82685_Small intestine_Duodenum | 4.1 |
| 97503_Patient-12pl_placenta | 0.9 | 90650_Adrenal_Adrenocortical | 2.4 |
| | | adenoma | |
| 94721_Donor 2 U - | 8.9 | 72410_Kidney_HRCE | 100.0 |
| A_Mesenchymal Stem Cells | | | |
| 94722_Donor 2 U - | 4.3 | 72411_Kidney_HRE | 36.1 |
| B_Mesenchymal Stem Cells | | | |
| 94723_Donor 2 U - | 9.1 | 73139_Uterus_Uterine smooth muscle | 20.6 |
| C_Mesenchymal Stem Cells | | cells | |

CNS_neurodegeneration_v1.0 Summary: Ag3304 This panel demonstrates the expression of this gene at low levels in the brain in several different individuals. However, no differential expression of this gene was detected between Alzheimer's diseased postmortem brains and those of non-demented controls in this experiment.

General_screening_panel_v1.4 Summary: Ag3304 Expression of this gene is low/undetectable (CTs>35) across all of the samples on this panel (data not shown).

General_screening_panel_v1.5 Summary: Ag3304 Results from two experiments using the same probe/primer set gave results that are only in moderate agreement. Only those results that are in agreement will be discussed here. Expression of the CG57670-01 gene is detected at low levels in pancreatic cancer cell line CAPAN2, CNS cancer cell line SF-295, lung cancer cell lines NCI-H460 and NCI-H23, and squamous cell carcinoma cell line SCC-4. Thus, expression of this gene can be used to distinguish these cell lines from the other samples on this panel. In addition, therapeutic modulation of the activity of this gene or its protein product, through the use of small molecule drugs, protein therapeutics or antibodies, might be beneficial in the treatment of lung cancer, pancreatic cancer, CNS cancer and squamous cell carcinoma.

In one experiment, this gene is expressed at low levels in the adrenal gland. As a glycolytic enzyme, activation of the CG57670-01 gene may increase oxidative metabolism in this tissue and be a treatment for Addison's disease and other adrenalopathies.

Panel 4D Summary: Ag3304 The CG57670-01 gene is expressed at low to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, and kidney. This ubiquitous pattern of expression suggests that this gene product may be involved in homeostatic processes for these and other cell types and tissues.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag3304 The CG57670-01 gene is expressed at moderate levels in islets of Langerhans (patient 1). Insulin secretion is dependent on glycolysis, and activation of this glycolytic enzyme may increase the glycolytic rate and insulin secretion in Type 2 diabetes. This gene is also expressed at low levels in adipose, cultured adipocytes, skeletal muscle, uterus and placenta. Therefore, activation of this enzyme may also be a treatment for obesity by increasing the glycolytic rate and energy expenditure in adipose tissue.

NOV36

Expression of NOV36A/CG57149-01 and NOV36B/CG57149-02 was assessed using the primer-probe set Ag3119, described in Table DA. Results of the RTQ-PCR runs are shown in Tables DB, DC, DD and DE. Please note that CG57149-02 represents a full-length physical clone of the CG57149-01 gene, validating the prediction of the gene sequence.

TABLE DA
Probe Name Ag3119

| Primers | Sequences | Length | Start Position | SEQ ID NO |
|---|---|---|---|---|
| Forward | 5′-tgtactaccagccatggtcaac-3′ | 22 | 12 | 365 |
| Probe | TET-5′-catgttcttcaacatcgccatcaaca-3′-TAMRA | 26 | 39 | 366 |
| Reverse | 5′-acttgtctgcaaacagttcgaa-3′ | 22 | 88 | 367 |

TABLE DB
CNS_neurodegeneration_v1.0

| | Rel. Exp. (%) Ag3119, Run | | Rel. Exp. (%) Ag3119, Run |
|---|---|---|---|
| Tissue Name | 208976882 | Tissue Name | 208976882 |
| AD 1 Hippo | 3.8 | Control (Path) 3 Temporal | 8.4 |
| | | Ctx | |
| AD 2 Hippo | 7.0 | Control (Path) 4 Temporal | 35.8 |
| | | Ctx | |
| AD 3 Hippo | 14.5 | AD 1 Occipital Ctx | 36.3 |
| AD 4 Hippo | 1.5 | AD 2 Occipital Ctx | 0.0 |
| | | (Missing) | |
| AD 5 Hippo | 100.0 | AD 3 Occipital Ctx | 18.4 |
| AD 6 Hippo | 4.8 | AD 4 Occipital Ctx | 7.1 |
| Control 2 Hippo | 1.7 | AD 5 Occipital Ctx | 0.7 |
| Control 4 Hippo | 1.2 | AD 6 Occipital Ctx | 36.3 |
| Control (Path) 3 Hippo | 2.2 | Control 1 Occipital Ctx | 0.6 |
| AD 1 Temporal Ctx | 16.4 | Control 2 Occipital Ctx | 3.7 |
| AD 2 Temporal Ctx | 12.2 | Control 3 Occipital Ctx | 49.0 |
| AD 3 Temporal Ctx | 16.6 | Control 4 Occipital Ctx | 3.0 |
| AD 4 Temporal Ctx | 19.8 | Control (Path) 1 Occipital | 29.9 |
| | | Ctx | |
| AD 5 Inf Temporal Ctx | 35.8 | Control (Path) 2 Occipital | 15.8 |
| | | Ctx | |
| AD 5 Sup Temporal Ctx | 41.2 | Control (Path) 3 Occipital | 7.4 |
| | | Ctx | |
| AD 6 Inf Temporal Ctx | 16.4 | Control (Path) 4 Occipital | 42.9 |
| | | Ctx | |
| AD 6 Sup Temporal Ctx | 35.6 | Control 1 Parietal Ctx | 10.4 |
| Control 1 Temporal Ctx | 7.9 | Control 2 Parietal Ctx | 40.9 |
| Control 2 Temporal Ctx | 0.0 | Control 3 Parietal Ctx | 5.2 |
| Control 3 Temporal Ctx | 4.7 | Control (Path) 1 Parietal | 5.2 |
| | | Ctx | |
| Control 3 Temporal Ctx | 4.0 | Control (Path) 2 Parietal | 8.9 |
| | | Ctx | |
| Control (Path) 1 Temporal | 14.9 | Control (Path) 3 Parietal | 5.7 |
| Ctx | | Ctx | |
| Control (Path) 2 Temporal | 20.0 | Control (Path) 4 Parietal | 44.1 |
| Ctx | | Ctx | |

TABLE DC
Panel 1.3D

| | Rel. Exp. (%) Ag3119, Run | | Rel. Exp. (%) Ag3119, Run |
|---|---|---|---|
| Tissue Name | 167985261 | Tissue Name | 167985261 |
| Liver adenocarcinoma | 1.2 | Kidney (fetal) | 16.3 |
| Pancreas | 0.6 | Renal ca. 786-0 | 7.1 |
| Pancreatic ca. CAPAN 2 | 27.9 | Renal ca. A498 | 0.0 |
| Adrenal gland | 0.9 | Renal ca. RXF 393 | 7.9 |
| Thyroid | 0.0 | Renal ca. ACHN | 26.6 |
| Salivary gland | 3.3 | Renal ca. UO-31 | 0.0 |
| Pituitary gland | 1.0 | Renal ca. TK-10 | 34.9 |
| Brain (fetal) | 100.0 | Liver | 5.5 |
| Brain (whole) | 3.2 | Liver (fetal) | 0.5 |
| Brain (amygdala) | 12.7 | Liver ca. (hepatoblast) | 44.4 |
| | | HepG2 | |
| Brain (cerebellum) | 68.3 | Lung | 6.7 |
| Brain (hippocampus) | 1.0 | Lung (fetal) | 0.0 |
| Brain (substantia nigra) | 5.1 | Lung ca. (small cell) LX-1 | 1.0 |
| Brain (thalamus) | 9.9 | Lung ca. (small cell) NCI- | 0.0 |
| | | H69 | |
| Cerebral Cortex | 6.7 | Lung ca. (s.cell var.) SHP- | 0.0 |
| | | 77 | |
| Spinal cord | 18.2 | Lung ca. (large cell)NCI- | 0.0 |
| | | H460 | |
| glio/astro U87-MG | 1.1 | Lung ca. (non-sm. cell) | 7.1 |
| | | A549 | |
| glio/astro U-118-MG | 1.5 | Lung ca. (non-s.cell) NCI- | 0.5 |
| | | H23 | |
| astrocytoma SW1783 | 2.1 | Lung ca. (non-s.cell) HOP- | 0.0 |
| | | 62 | |
| neuro*; met SK-N-AS | 1.4 | Lung ca. (non-s.cl) NCI- | 2.0 |
| | | H522 | |
| astrocytoma SF-539 | 0.0 | Lung ca. (squam.) SW 900 | 0.0 |
| astrocytoma SNB-75 | 1.5 | Lung ca. (squam.) NCI- | 0.0 |
| | | H596 | |
| glioma SNB-19 | 0.0 | Mammary gland | 1.0 |
| glioma U251 | 0.0 | Breast ca.* (pl.ef) MCF-7 | 0.0 |
| glioma SF-295 | 6.5 | Breast ca.* (pl.ef) MDA- | 0.0 |
| | | MB-231 | |
| Heart (fetal) | 0.0 | Breast ca.* (pl.ef) T47D | 0.0 |
| Heart | 8.1 | Breast ca. BT-549 | 5.6 |
| Skeletal muscle (fetal) | 9.9 | Breast ca. MDA-N | 0.0 |
| Skeletal muscle | 4.1 | Ovary | 0.0 |
| Bone marrow | 3.1 | Ovarian ca. OVCAR-3 | 0.0 |
| Thymus | 97.3 | Ovarian ca. OVCAR-4 | 0.0 |
| Spleen | 9.5 | Ovarian ca. OVCAR-5 | 0.0 |
| Lymph node | 19.8 | Ovarian ca. OVCAR-8 | 0.7 |
| Colorectal | 24.5 | Ovarian ca. IGROV-1 | 0.0 |
| Stomach | 18.8 | Ovarian ca.* (ascites) SK- | 11.5 |
| | | OV-3 | |
| Small intestine | 22.7 | Uterus | 10.8 |
| Colon ca. SW480 | 0.0 | Placenta | 0.0 |
| Colon ca.* SW620(SW480 | 4.3 | Prostate | 5.6 |
| met) | | | |
| Colon ca. HT29 | 0.0 | Prostate ca.* (bone met)PC-3 | 0.0 |
| Colon ca. HCT-116 | 2.1 | Testis | 4.3 |
| Colon ca. CaCo-2 | 16.4 | Melanoma Hs688(A).T | 0.0 |
| Colon ca. tissue(ODO3866) | 0.0 | Melanoma* (met) | 0.5 |
| | | Hs688(B).T | |
| Colon ca. HCC-2998 | 0.0 | Melanoma UACC-62 | 0.0 |
| Gastric ca.* (liver met) NCI- | 0.0 | Melanoma M14 | 0.0 |
| N87 | | | |
| Bladder | 1.7 | Melanoma LOX IMVI | 0.0 |
| Trachea | 1.6 | Melanoma* (met) SK- | 0.0 |
| | | MEL-5 | |
| Kidney | 0.0 | Adipose | 0.0 |

TABLE DD
Panel 4D

| | Rel. Exp. (%) Ag3119, Run | | Rel. Exp. (%) Ag3119, Run |
|---|---|---|---|
| Tissue Name | 164526109 | Tissue Name | 164526109 |
| Secondary Th1 act | 5.7 | HUVEC IL-1beta | 3.3 |
| Secondary Th2 act | 8.8 | HUVEC IFN gamma | 19.5 |
| Secondary Tr1 act | 11.3 | HUVEC TNF alpha + IFN gamma | 34.6 |
| Secondary Th1 rest | 9.2 | HUVEC TNF alpha + IL4 | 10.8 |
| Secondary Th2 rest | 12.9 | HUVEC IL-11 | 14.2 |
| Secondary Tr1 rest | 18.9 | Lung Microvascular EC none | 36.3 |
| Primary Th1 act | 1.5 | Lung Microvascular EC TNF alpha + | 31.9 |
| | | IL-1beta | |
| Primary Th2 act | 7.2 | Microvascular Dermal EC none | 38.7 |
| Primary Tr1 act | 5.9 | Microsvasular Dermal EC | 16.4 |
| | | TNF alpha + IL-1beta | |
| Primary Th1 rest | 56.6 | Bronchial epithelium TNF alpha + | 0.0 |
| | | IL1beta | |
| Primary Th2 rest | 41.8 | Small airway epithelium none | 2.3 |
| Primary Tr1 rest | 31.2 | Small airway epithelium TNF alpha + | 1.6 |
| | | IL-1beta | |
| CD45RA CD4 lymphocyte | 4.4 | Coronery artery SMC rest | 5.4 |
| act | | | |
| CD45RO CD4 lymphocyte | 8.5 | Coronery artery SMC TNF alpha + | 0.9 |
| act | | IL-1beta | |
| CD8 lymphocyte act | 3.6 | Astrocytes rest | 14.8 |
| Secondary CD8 lymphocyte | 5.3 | Astrocytes TNF alpha + IL-1beta | 3.4 |
| rest | | | |
| Secondary CD8 lymphocyte | 6.9 | KU-812 (Basophil) rest | 0.3 |
| act | | | |
| CD4 lymphocyte none | 8.1 | KU-812 (Basophil) | 0.7 |
| | | PMA/ionomycin | |
| 2ry Th1/Th2/Tr1_anti-CD95 | 20.0 | CCD1106 (Keratinocytes) none | 2.5 |
| CH11 | | | |
| LAK cells rest | 4.7 | CCD1106 (Keratinocytes) | 1.9 |
| | | TNF alpha + IL-1beta | |
| LAK cells IL-2 | 6.0 | Liver cirrhosis | 5.3 |
| LAK cells IL-2 + IL-12 | 11.8 | Lupus kidney | 1.6 |
| LAK cells IL-2 + IFN gamma | 17.6 | NCI-H292 none | 1.8 |
| LAK cells IL-2 + IL-18 | 19.3 | NCI-H292 IL-4 | 0.2 |
| LAK cells PMA/ionomycin | 2.8 | NCI-H292 IL-9 | 3.0 |
| NK Cells IL-2 rest | 22.8 | NCI-H292 IL-13 | 0.4 |
| Two Way MLR 3 day | 21.2 | NCI-H292 IFN gamma | 2.5 |
| Two Way MLR 5 day | 7.7 | HPAEC none | 16.7 |
| Two Way MLR 7 day | 3.4 | HPAEC TNF alpha + IL-1beta | 11.0 |
| PBMC rest | 1.1 | Lung fibroblast none | 9.9 |
| PBMC PWM | 15.7 | Lung fibroblast TNF alpha + IL- | 0.6 |
| | | 1beta | |
| PBMC PHA-L | 4.9 | Lung fibroblast IL-4 | 5.6 |
| Ramos (B cell) none | 0.0 | Lung fibroblast IL-9 | 9.0 |
| Ramos (B cell) ionomycin | 0.0 | Lung fibroblast IL-13 | 8.1 |
| B lymphocytes PWM | 0.7 | Lung fibroblast IFN gamma | 10.4 |
| B lymphocytes CD40L and | 1.2 | Dermal fibroblast CCD1070 rest | 2.2 |
| IL-4 | | | |
| EOL-1 dbcAMP | 0.0 | Dermal fibroblast CCD1070 TNF | 35.1 |
| | | alpha | |
| EOL-1 dbcAMP | 0.0 | Dermal fibroblast CCD1070 IL- | 3.0 |
| PMA/ionomycin | | 1beta | |
| Dendritic cells none | 0.2 | Dermal fibroblast IFN gamma | 3.9 |
| Dendritic cells LPS | 0.0 | Dermal fibroblast IL-4 | 10.7 |
| Dendritic cells anti-CD40 | 0.0 | IBD Colitis 2 | 0.9 |
| Monocytes rest | 0.0 | IBD Crohn's | 1.9 |
| Monocytes LPS | 11.5 | Colon | 7.2 |
| Macrophages rest | 8.0 | Lung | 0.0 |
| Macrophages LPS | 0.0 | Thymus | 12.4 |
| HUVEC none | 20.3 | Kidney | 100.0 |
| HUVEC starved | 82.4 | | |

TABLE DE
Panel CNS_1

| | Rel. Exp. (%) Ag3119, Run | | Rel. Exp. (%) Ag3119, Run |
|---|---|---|---|
| Tissue Name | 182257490 | Tissue Name | 182257490 |
| BA4 Control | 2.8 | BA17 PSP | 2.6 |
| BA4 Control2 | 0.0 | BA17 PSP2 | 0.0 |
| BA4 Alzheimer's2 | 2.2 | Sub Nigra Control | 1.3 |
| BA4 Parkinson's | 31.0 | Sub Nigra Control2 | 11.3 |
| BA4 Parkinson's2 | 7.2 | Sub Nigra Alzheimer's2 | 5.7 |
| BA4 Huntington's | 26.8 | Sub Nigra Parkinson's2 | 4.7 |
| BA4 Huntington's2 | 5.9 | Sub Nigra Huntington's | 28.7 |
| BA4 PSP | 2.0 | Sub Nigra Huntington's2 | 1.1 |
| BA4 PSP2 | 4.6 | Sub Nigra PSP2 | 0.0 |
| BA4 Depression | 14.5 | Sub Nigra Depression | 4.6 |
| BA4 Depression2 | 6.8 | Sub Nigra Depression2 | 0.0 |
| BA7 Control | 48.0 | Glob Palladus Control | 8.1 |
| BA7 Control2 | 0.0 | Glob Palladus Control2 | 8.2 |
| BA7 Alzheimer's2 | 16.3 | Glob Palladus Alzheimer's | 6.1 |
| BA7 Parkinson's | 6.7 | Glob Palladus | 6.9 |
| | | Alzheimer's2 | |
| BA7 Parkinson's2 | 30.1 | Glob Palladus Parkinson's | 44.1 |
| BA7 Huntington's | 2.8 | Glob Palladus | 19.6 |
| | | Parkinson's2 | |
| BA7 Huntington's2 | 75.8 | Glob Palladus PSP | 0.0 |
| BA7 PSP | 2.4 | Glob Palladus PSP2 | 0.0 |
| BA7 PSP2 | 3.6 | Glob Palladus Depression | 0.0 |
| BA7 Depression | 36.3 | Temp Pole Control | 7.1 |
| BA9 Control | 3.3 | Temp Pole Control2 | 2.5 |
| BA9 Control2 | 23.7 | Temp Pole Alzheimer's | 6.4 |
| BA9 Alzheimer's | 8.4 | Temp Pole Alzheimer's2 | 9.1 |
| BA9 Alzheimer's2 | 9.2 | Temp Pole Parkinson's | 29.1 |
| BA9 Parkinson's | 22.5 | Temp Pole Parkinson's2 | 2.8 |
| BA9 Parkinson's2 | 10.4 | Temp Pole Huntington's | 28.5 |
| BA9 Huntington's | 20.3 | Temp Pole PSP | 8.2 |
| BA9 Huntington's2 | 35.8 | Temp Pole PSP2 | 0.0 |
| BA9 PSP | 5.9 | Temp Pole Depression2 | 0.0 |
| BA9 PSP2 | 0.0 | Cing Gyr Control | 18.4 |
| BA9 Depression | 0.0 | Cing Gyr Control2 | 4.5 |
| BA9 Depression2 | 0.0 | Cing Gyr Alzheimer's | 5.3 |
| BA17 Control | 60.7 | Cing Gyr Alzheimer's2 | 15.5 |
| BA17 Control2 | 31.0 | Cing Gyr Parkinson's | 3.6 |
| BA17 Alzheimer's2 | 36.9 | Cing Gyr Parkinson's2 | 30.6 |
| BA17 Parkinson's | 40.3 | Cing Gyr Huntington's | 1.6 |
| BA17 Parkinson's2 | 100.0 | Cing Gyr Huntington's2 | 1.6 |
| BA17 Huntington's | 1.8 | Cing Gyr PSP | 0.0 |
| BA17 | 37.1 | Cing Gyr PSP2 | 14.5 |
| Huntington's2 | | | |
| BA17 Depression | 7.2 | Cing Gyr Depression | 0.0 |
| BA17 Depression2 | 4.3 | Cing Gyr Depression2 | 5.8 |

CNS_neurodegeneration_v1.0 Summary: Ag3119 This panel confirms the expression of this gene at low levels in the brain in an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag3119 The CG57149-01 gene is expressed at highest levels in fetal brain (CT=32.2). Interestingly, this gene is expressed at much lower levels in adult brain (CT=37.2). Therefore, expression of this gene can be used to distinguish fetal brain from adult brain. In addition, the relative overexpression of this gene in fetal brain suggests that this protein product may enhance brain growth or development in the fetus and thus may also act in a regenerative capacity in the adult.

This gene is also expressed at relatively high levels in thymus. The CG57149-01 gene encodes a protein with homology to cis/trans peptidyl prolyl isomerase. peptidyl-prolyl cis/trans isomerase A (also known as PPIA or cyclophilin A) is a member of the immunophilin class of proteins. These cytosolic enzymes have peptidyl-prolyl cis/trans isomerase activity and are thought to play a role in protein folding and/or intracellular protein transport (ref. 1). The immunosuppressive action of the immunomodulatory agent cyclosporin is linked to its binding to cyclophilin, which inhibits the enzymatic acticity of cyclophilin (ref. 2). Cyclosporin has major effects on T cells. Modulation of this novel cyclophilin may therefore be important in the treatment of in regulating T cell function and treating T cell mediated diseases such as asthma, arthritis, psoriasis, IBD, and systemic lupus erythematosus.

References:

1. Ivery M T. Immunophilins: switched on protein binding domains? Med Res Rev 2000 November;20(6):452-84.

Peptidylprolyl isomerases (PPIases) are a group of cytosolic enzymes first characterized by their ability to catalyze the cis-trans isomerization of cis-peptidylprolyl bonds. Subsequently, some PPIases were also identified as the initial targets of the immunosuppressant drugs-cyclosporin A (CsA), FK506, and rapamycin-have been called immunophilins. Immunophilins have been found to be both widely distributed and abundantly expressed leading to suggestions that they may play a general role in cellular biochemistry. However, the nature of this role has been difficult to elucidate and is still controversial in vivo. A number of roles for these enzymes have been identified in vitro including the ability to catalyze the refolding of partly denatured proteins and stabilize multiprotein complexes such as Ca(2+) channels, inactive steroid receptor complexes, and receptor protein tyrosine kinases. Generally, these effects appear to depend on the ability of immunophilins to selectively bind to other proteins. This review will examine in detail experimental and structural investigations of the mechanism of PPIase activity for both FKBPs and cyclophilins and suggest a mechanism for these enzymes, which depends on their ability to recognize a specific peptide conformation rather than sequence. Examination of structures of immunophilin-protein complexes will then be used to further suggest that the ability of these enzymes to recognize specific peptide conformations is central to the formation of these complexes and may constitute a general function of immunophilin enzymes. The binding of ligand to immunophilins will also be shown to stabilize specific conformations in surface loops of these proteins that are observed to play a critical role in a number of immunophilin-protein complexes suggesting that the immunophilins may constitute a class of ligand-triggered selective protein binders.

PMID: 11058892

2. Russell G, Graveley R, Seid J, al-Humidan A K, Skjodt H. Mechanisms of action of cyclosporine and effects on connective tissues. Semin Arthritis Rheum 1992 June;21(6 Suppl 3):16-22.

Cyclosporine is a potent immunomodulatory agent with an increasing number of clinical applications. Its major mode of action is inhibition of the production of cytokines involved in the regulation of T-cell activation. In particular, cyclosporine inhibits the transcription of interleukin 2. Although cyclosporine's major actions are on T cells, there is some evidence that it produces direct effects on other cell types. Its immunosuppressive action is closely linked to its binding of cyclophilin, a member of a family of high-affinity cyclosporine-binding proteins widely distributed in different cell types and in different species. The cyclophilins have been shown to have peptidyl-prolyl cis-trans isomerase enzyme activity that is blocked by cyclosporine. Although this may be a factor in cyclosporine's selective inhibition of cytokine gene transcription, it is still unclear whether inhibition of this activity is the mechanism through which cyclosporine exerts its effects on target cells. The ubiquitous presence of cyclophilins raises the question of why cyclosporine has major effects on T cells. Perhaps the critical proteins affected are transcriptional regulators restricted in their tissue distribution. The effects of cyclosporine on r cells and, directly or indirectly, on connective tissue cells, all of which can produce a range of cytokines, are of interest in relation to the tissue changes that occur in such inflammatory conditions as rheumatoid arthritis.

PMID: 1502562

Panel 4D Summary: Ag3119 The CG57149-01 gene is expressed at moderate levels in T cells and is also expressed at significant levels in endothelium, epithelium and fibroblasts as well as in normal thymus and kidney. Expression of this gene in thymus is consistent with what is observed in Panel 1.3D. Furthermore, treatment of proinflammatory mediators reduces the level of this transcript in HUVEC cells as compared to in starved cells. The protein encoded for by this transcript may be important in the normal function of the cell types that express it. Regulation of the protein encoded for by this transcript could be important for maintaining or resuming normal homeostasis.

Panel CNS_{—}1 Summary: Ag3119 This panel confirms the expression of this gene at low levels in the brain in an independent group of individuals. Please see Panel 1.3D for a discussion of the potential utility of this gene in treatment of central nervous system disorders.

NOV37

Expression of NOV37/CG57151-01 was assessed using the primer-probe set Ag3120, described in Table AEA. Results of the RTQ-PCR runs are shown in Tables AEB, AEC, AED and AEE.

TABLE AEA
Probe Name Ag3120

| Primers | Sequences | Length | Start Position | SEQ ID NO |
|---|---|---|---|---|
| Forward | 5′-atgctctgagcactggagaa-3′ | 20 | 215 | 368 |
| Probe | TET-5′-tcctgctttcacagaattattccaggg-3′-TAMRA | 27 | 256 | 369 |
| Reverse | 5′-gtgtgaagtcaccactctgaca-3′ | 22 | 289 | 370 |

TABLE AEB
CNS_neurodegeneration_v1.0

| | Rel. Exp. (%) Ag3120, Run | | Rel. Exp. (%) Ag3120, Run |
|---|---|---|---|
| Tissue Name | 208976883 | Tissue Name | 208976883 |
| AD 1 Hippo | 18.6 | Control (Path) 3 Temporal | 6.2 |
| | | Ctx | |
| AD 2 Hippo | 26.1 | Control (Path) 4 Temporal | 46.0 |
| | | Ctx | |
| AD 3 Hippo | 10.1 | AD 1 Occipital Ctx | 26.4 |
| AD 4 Hippo | 10.2 | AD 2 Occipital Ctx | 0.0 |
| | | (Missing) | |
| AD 5 hippo | 90.1 | AD 3 Occipital Ctx | 8.9 |
| AD 6 Hippo | 67.8 | AD 4 Occipital Ctx | 24.5 |
| Control 2 Hippo | 35.1 | AD 5 Occipital Ctx | 47.3 |
| Control 4 Hippo | 16.8 | AD 6 Occipital Ctx | 36.6 |
| Control (Path) 3 Hippo | 12.0 | Control 1 Occipital Ctx | 6.5 |
| AD 1 Temporal Ctx | 35.8 | Control 2 Occipital Ctx | 74.7 |
| AD 2 Temporal Ctx | 41.5 | Control 3 Occipital Ctx | 36.6 |
| AD 3 Temporal Ctx | 10.3 | Control 4 Occipital Ctx | 8.7 |
| AD 4 Temporal Ctx | 25.0 | Control (Path) 1 Occipital | 100.0 |
| | | Ctx | |
| AD 5 Inf Temporal Ctx | 87.1 | Control (Path) 2 Occipital | 17.1 |
| | | Ctx | |
| AD 5 Sup Temporal Ctx | 57.8 | Control (Path) 3 Occipital | 5.1 |
| | | Ctx | |
| AD 6 Inf Temporal Ctx | 79.6 | Control (Path) 4 Occipital | 37.9 |
| | | Ctx | |
| AD 6 Sup Temporal Ctx | 97.9 | Control 1 Parietal Ctx | 13.1 |
| Control 1 Temporal Ctx | 12.2 | Control 2 Parietal Ctx | 44.8 |
| Control 2 Temporal Ctx | 43.2 | Control 3 Parietal Ctx | 19.5 |
| Control 3 Temporal Ctx | 26.6 | Control (Path) 1 Parietal | 65.5 |
| | | Ctx | |
| Control 4 Temporal Ctx | 13.5 | Control (Path) 2 Parietal | 40.6 |
| | | Ctx | |
| Control (Path) 1 Temporal | 59.5 | Control (Path) 3 Parietal | 3.9 |
| Ctx | | Ctx | |
| Control (Path) 2 Temporal | 47.3 | Control (Path) 4 Parietal | 56.3 |
| Ctx | | Ctx | |

TABLE AEC
Panel 1.3D

| | Rel. Exp. (%) Ag3120, Run | | Rel. Exp. (%) Ag3120, Run |
|---|---|---|---|
| Tissue Name | 167985266 | Tissue Name | 167985266 |
| Liver adenocarcinoma | 46.0 | Kidney (fetal) | 97.3 |
| Pancreas | 14.3 | Renal ca. 786-0 | 25.7 |
| Pancreatic ca. CAPAN 2 | 8.2 | Renal ca. A498 | 26.2 |
| Adrenal gland | 12.8 | Renal ca. RXF 393 | 43.5 |
| Thyroid | 8.4 | Renal ca. ACHN | 12.0 |
| Salivary gland | 3.2 | Renal ca. UO-31 | 29.5 |
| Pituitary gland | 16.8 | Renal ca. TK-10 | 36.9 |
| Brain (fetal) | 30.1 | Liver | 8.7 |
| Brain (whole) | 12.4 | Liver (fetal) | 18.7 |
| Brain (amygdala) | 20.0 | Liver ca. (hepatoblast) | 42.0 |
| | | HepG2 | |
| Brain (cerebellum) | 30.8 | Lung | 12.2 |
| Brain (hippocampus) | 16.4 | Lung (fetal) | 45.7 |
| Brain (substantia nigra) | 42.3 | Lung ca. (small cell) LX-1 | 26.2 |
| Brain (thalamus) | 3.0 | Lung ca. (small cell) NCI- | 10.1 |
| | | H69 | |
| Cerebral Cortex | 12.4 | Lung ca. (s.cell var.) SHP- | 85.3 |
| | | 77 | |
| Spinal cord | 24.5 | Lung ca. (large cell)NCI- | 4.2 |
| | | H460 | |
| glio/astro U87-MG | 20.4 | Lung ca. (non-sm. cell) | 32.5 |
| | | A549 | |
| glio/astro U-118-MG | 24.7 | Lung ca. (non-s.cell) NCI- | 26.4 |
| | | H23 | |
| astrocytoma SW1783 | 16.5 | Lung ca. (non-s.cell) HOP- | 15.0 |
| | | 62 | |
| neuro*; met SK-N-AS | 21.9 | Lung ca. (non-s.cl) NCI- | 15.6 |
| | | H522 | |
| astrocytoma SF-539 | 21.6 | Lung ca. (squam.) SW 900 | 26.8 |
| astrocytoma SNB-75 | 28.3 | Lung ca. (squam.) NCI- | 33.7 |
| | | H596 | |
| glioma SNB-19 | 12.8 | Mammary gland | 6.7 |
| glioma U251 | 47.0 | Breast ca.* (pl.ef) MCF-7 | 26.1 |
| glioma SF-295 | 13.3 | Breast ca.* (pl.ef) MDA- | 22.7 |
| | | MB-231 | |
| Heart (fetal) | 8.7 | Breast ca.* (pl.ef) T47D | 43.5 |
| Heart | 11.6 | Breast ca. BT-549 | 16.4 |
| Skeletal muscle (fetal) | 5.4 | Breast ca. MDA-N | 25.0 |
| Skeletal muscle | 10.8 | Ovary | 5.2 |
| Bone marrow | 26.4 | Ovarian ca. OVCAR-3 | 13.5 |
| Thymus | 49.7 | Ovarian ca. OVCAR-4 | 21.3 |
| Spleen | 49.7 | Ovarian ca. OVCAR-5 | 100.0 |
| Lymph node | 37.4 | Ovarian ca. OVCAR-8 | 6.8 |
| Colorectal | 7.3 | Ovarian ca. IGROV-1 | 8.0 |
| Stomach | 12.7 | Ovarian ca.* (ascites) SK- | 56.3 |
| | | OV-3 | |
| Small intestine | 14.6 | Uterus | 16.7 |
| Colon ca. SW480 | 29.5 | Placenta | 5.1 |
| Colon ca.* SW620(SW480 | 52.1 | Prostate | 11.1 |
| met) | | | |
| Colon ca. HT29 | 23.0 | Prostate ca.* (bone met)PC-3 | 22.2 |
| Colon ca. HCT-116 | 23.8 | Testis | 8.4 |
| Colon ca. CaCo-2 | 48.0 | Melanoma Hs688(A).T | 4.5 |
| Colon ca. tissue(ODO3866) | 32.5 | Melanoma* (met) | 4.1 |
| | | Hs688(B).T | |
| Colon ca. HCC-2998 | 32.5 | Melanoma UACC-62 | 13.0 |
| Gastric ca.* (liver met) NCI- | 27.4 | Melanoma M14 | 11.0 |
| N87 | | | |
| Bladder | 34.2 | Melanoma LOX IMVI | 15.4 |
| Trachea | 12.3 | Melanoma* (met) SK- | 12.6 |
| | | MEL-5 | |
| Kidney | 22.7 | Adipose | 44.4 |

TABLE AED
Panel 4D

| | Rel. Exp. (%) Ag3120, Run | | Rel. Exp. (%) Ag3120, Run |
|---|---|---|---|
| Tissue Name | 164526129 | Tissue Name | 164526129 |
| Secondary Th1 act | 15.5 | HUVEC IL-1beta | 7.1 |
| Secondary Th2 act | 17.6 | HUVEC IFN gamma | 19.3 |
| Secondary Tr1 act | 21.0 | HUVEC TNF alpha + IFN gamma | 12.4 |
| Secondary Th1 rest | 9.0 | HUVEC TNF alpha + IL4 | 16.0 |
| Secondary Th2 rest | 10.7 | HUVEC IL-11 | 20.7 |
| Secondary Tr1 rest | 16.8 | Lung Microvascular EC none | 12.1 |
| Primary Th1 act | 17.3 | Lung Microvascular EC TNF alpha + | 11.9 |
| | | IL-1beta | |
| Primary Th2 act | 15.7 | Microvascular Dermal EC none | 14.0 |
| Primary Tr1 act | 23.8 | Microsvasular Dermal EC | 7.6 |
| | | TNF alpha + IL-1beta | |
| Primary Th1 rest | 50.0 | Bronchial epithelium TNF alpha + | 18.0 |
| | | IL1beta | |
| Primary Th2 rest | 33.2 | Small airway epithelium none | 7.0 |
| Primary Tr1 rest | 26.8 | Small airway epithelium TNF alpha + | 34.4 |
| | | IL-1beta | |
| CD45RA CD4 lymphocyte | 9.8 | Coronery artery SMC rest | 8.1 |
| act | | | |
| CD45RO CD4 lymphocyte | 21.3 | Coronery artery SMC TNF alpha + | 5.0 |
| act | | IL-1beta | |
| CD8 lymphocyte act | 23.7 | Astrocytes rest | 4.9 |
| Secondary CD8 lymphocyte | 23.2 | Astrocytes TNF alpha + IL-1beta | 7.0 |
| rest | | | |
| Secondary CD8 lymphocyte | 10.2 | KU-812 (Basophil) rest | 8.3 |
| act | | | |
| CD4 lymphocyte none | 12.9 | KU-812 (Basophil) | 19.2 |
| | | PMA/ionomycin | |
| 2ry Th1/Th2/Tr1_anti-CD95 | 25.3 | CCD1106 (Keratinocytes) none | 10.3 |
| CH11 | | | |
| LAK cells rest | 18.8 | CCD1106 (Keratinocytes) | 5.4 |
| | | TNF alpha + IL-1beta | |
| LAK cells IL-2 | 19.9 | Liver cirrhosis | 5.4 |
| LAK cells IL-2 + IL-12 | 20.0 | Lupas kidney | 3.2 |
| LAK cells IL-2 + IFN gamma | 26.1 | NCI-H292 none | 40.6 |
| LAK cells IL-2 + IL-18 | 23.3 | NCI-H292 IL-4 | 33.7 |
| LAK cells PMA/ionomycin | 29.3 | NCI-H292 IL-9 | 33.2 |
| NK Cells IL-2 rest | 20.4 | NCI-H292 IL-13 | 23.0 |
| Two Way MLR 3 day | 27.9 | NCI-H292 IFN gamma | 21.0 |
| Two Way MLR 5 day | 12.9 | HPAEC none | 10.7 |
| Two Way MLR 7 day | 11.3 | HPAEC TNF alpha + IL-1beta | 12.6 |
| PBMC rest | 35.4 | Lung fibroblast none | 4.7 |
| PBMC PWM | 50.0 | Lung fibroblast TNF alpha + IL- | 6.1 |
| | | 1beta | |
| PBMC PHA-L | 19.3 | Lung fibroblast IL-4 | 10.2 |
| Ramos (B cell) none | 26.6 | Lung fibroblast IL-9 | 11.1 |
| Ramos (B cell) ionomycin | 49.0 | Lung fibroblast IL-13 | 8.8 |
| B lymphocytes PWM | 43.5 | Lung fibroblast IFN gamma | 11.0 |
| B lymphocytes CD40L and | 14.6 | Dermal fibroblast CCD1070 rest | 17.8 |
| IL-4 | | | |
| EOL-1 dbcAMP | 19.9 | Dermal fibroblast CCD1070 TNF | 40.9 |
| | | alpha | |
| EOL-1 dbcAMP | 26.2 | Dermal fibroblast CCD1070 IL- | 8.6 |
| PMA/ionomycin | | 1beta | |
| Dendritic cells none | 14.1 | Dermal fibroblast IFN gamma | 12.0 |
| Dendritic cells LPS | 11.3 | Dermal fibroblast IL-4 | 8.2 |
| Dendritic cells anti-CD40 | 11.2 | IBD Colitis 2 | 2.5 |
| Monocytes rest | 100.0 | IBD Crohn's | 3.1 |
| Monocytes LPS | 21.8 | Colon | 10.2 |
| Macrophages rest | 14.6 | Lung | 8.7 |
| Macrophages LPS | 13.0 | Thymus | 22.7 |
| HUVEC none | 17.7 | Kidney | 44.8 |
| HUVEC starved | 22.2 | | |

TABLE AEE
Panel CNS_1

| | Rel. Exp. (%) Ag3120, Run | | Rel. Exp. (%) Ag3120, Run |
|---|---|---|---|
| Tissue Name | 182257530 | Tissue Name | 182257530 |
| BA4 Control | 23.7 | BA17 PSP | 26.8 |
| BA4 Control2 | 40.9 | BA17 PSP2 | 12.2 |
| BA4 Alzheimer's2 | 3.1 | Sub Nigra Control | 50.3 |
| BA4 Parkinson's | 90.1 | Sub Nigra Control2 | 30.6 |
| BA4 Parkinson's2 | 73.2 | Sub Nigra Alzheimer's2 | 8.1 |
| BA4 Huntington's | 46.0 | Sub Nigra Parkinson's2 | 86.5 |
| BA4 Huntington's2 | 9.0 | Sub Nigra Huntington's | 76.8 |
| BA4 PSP | 8.7 | Sub Nigra Huntington's2 | 87.7 |
| BA4 PSP2 | 25.9 | Sub Nigra PSP2 | 15.1 |
| BA4 Depression | 31.4 | Sub Nigra Depression | 18.8 |
| BA4 Depression2 | 28.3 | Sub Nigra Depression2 | 16.2 |
| BA7 Control | 66.4 | Glob Palladus Control | 34.2 |
| BA7 Control2 | 64.6 | Glob Palladus Control2 | 12.5 |
| BA7 Alzheimer's2 | 11.5 | Glob Palladus Alzheimer's | 9.9 |
| BA7 Parkinson's | 26.8 | Glob Palladus | 12.8 |
| | | Alzheimer's2 | |
| BA7 Parkinson's2 | 49.3 | Glob Palladus Parkinson's | 95.9 |
| BA7 Huntington's | 50.7 | Glob Palladus | 15.4 |
| | | Parkinson's2 | |
| BA7 Huntington's2 | 66.4 | Glob Palladus PSP | 12.9 |
| BA7 PSP | 70.7 | Glob Palladus PSP2 | 10.1 |
| BA7 PSP2 | 23.7 | Glob Palladus Depression | 7.3 |
| BA7 Depression | 24.1 | Temp Pole Control | 14.9 |
| BA9 Control | 16.7 | Temp Pole Control2 | 39.2 |
| BA9 Control2 | 78.5 | Temp Pole Alzheimer's | 10.5 |
| BA9 Alzheimer's | 11.2 | Temp Pole Alzheimer's2 | 11.4 |
| BA9 Alzheimer's2 | 38.7 | Temp Pole Parkinson's | 30.8 |
| BA9 Parkinson's | 33.2 | Temp Pole Parkinson's2 | 40.3 |
| BA9 Parkinson's2 | 88.3 | Temp Pole Huntington's | 65.5 |
| BA9 Huntington's | 37.6 | Temp Pole PSP | 3.3 |
| BA9 Huntington's2 | 20.4 | Temp Pole PSP2 | 7.6 |
| BA9 PSP | 23.3 | TemP Pole Depression2 | 18.8 |
| BA9 PSP2 | 2.7 | Cing Gyr Control | 47.6 |
| BA9 Depression | 13.0 | Cing Gyr Control2 | 26.1 |
| BA9 Depression2 | 12.1 | Cing Gyr Alzheimer's | 22.2 |
| BA17 Control | 88.3 | Cing Gyr Alzheimer's2 | 23.0 |
| BA17 Control2 | 82.9 | Cing Gyr Parkinson's | 43.2 |
| BA17 Alzheimer's2 | 35.1 | Cing Gyr Parkinson's2 | 37.4 |
| BA17 Parkinson's | 99.3 | Cing Gyr Huntington's | 60.3 |
| BA17 Parkinson's2 | 100.0 | Cing Gyr Huntington's2 | 39.8 |
| BA17 Huntington's | 45.7 | Cing Gyr PSP | 38.2 |
| BA17 | 40.3 | Cing Gyr PSP2 | 10.2 |
| Huntington's2 | | | |
| BA17 Depression | 38.7 | Cing Gyr Depression | 21.5 |
| BA17 Depression2 | 48.3 | Cing Gyr Depression2 | 16.7 |

CNS_neurodegeneration_v1.0 Summary: Ag3120 This panel confirms the expression of this gene at moderate levels in the brain in an independent group of individuals. However, no differential expression of this gene was detected between Alzheimer's diseased postmortem brains and those of non-demented controls In this experiment. Please see Panel 1.3D for a discussion of the potential utility of this gene in treatment of central nervous system disorders.

Panel 1.3D Summary: Ag3120 The CG57151-01 gene is expressed at moderate to low levels in all tissues on this panel, with the highest expression seen in ovarian cancer cell line OVCAR-5 (CT=29.1). This gene is expressed at moderate levels throughout the central nervous system, including in amygdala, hippocampus, cerebral cortex, cerebellum, substantia nigra, and spinal cord, with lower expression detected in thalamus. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at low to moderate levels in adrenal gland, thyroid, pituitary gland, pancreas, adipose, skeletal muscle, heart and liver. Therefore, therapeutic modulation of the activity of this gene may prove useful in the treatment of endocrine and metabolically related diseases, such as obesity and diabetes.

This gene encodes a protein with homology to peptidyl-prolyl cis/trans isomerase A (PPIA or cyclophilin A), a cytosolic enzyme involved in protein folding and/or intracellular protein transport. Cyclophilin genes are expressed in numerous tissues. The interaction of cyclophilin and cyclosporine has an effect on cytokine production in T-cells. Therefore, modulation of this gene may be used in the treatment of inflamatory diseases such as rheumatoid arthritis.

References:

1. Russell G, Graveley R, Seid J, al-Humidan A K, Skjodt H. Mechanisms of action of cyclosporine and effects on connective tissues. Semin Arthritis Rheum 1992 June;21(6 Suppl 3):16-22.

Cyclosporine is a potent immunomodulatory agent with an increasing number of clinical applications. Its major mode of action is inhibition of the production of cytokines involved in the regulation of T-cell activation. In particular, cyclosporine inhibits the transcription of interleukin 2. Although cyclosporine's major actions are on T cells, there is some evidence that it produces direct effects on other cell types. Its immunosuppressive action is closely linked to its binding of cyclophilin, a member of a family of high-affinity cyclosporine-binding proteins widely distributed in different cell types and in different species. The cyclophilins have been shown to have peptidyl-prolyl cis-trans isomerase enzyme activity that is blocked by cyclosporine. Although this may be a factor in cyclosporine's selective inhibition of cytokine gene transcription, it is still unclear whether inhibition of this activity is the mechanism through which cyclosporine exerts its effects on target cells. The ubiquitous presence of cyclophilins raises the question of why cyclosporine has major effects on T cells. Perhaps the critical proteins affected are transcriptional regulators restricted in their tissue distribution. The effects of cyclosporine on T cells and, directly or indirectly, on connective tissue cells, all of which can produce a range of cytokines, are of interest in relation to the tissue changes that occur in such inflammatory conditions as rheumatoid arthritis.

PMID: 1502562

Panel 4D Summary: Ag3120 The CG57151-01 gene is expressed at high to moderate levels in a wide range of cell types of significance in the immune response in health and disease. These cells include members of the T-cell, B-cell, endothelial cell, macrophage/monocyte/dendritic cell, basophil, eosinophil, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues.

This pattern is in agreement with the expression profile in Panel 1.3D and also suggests a role for the gene product in cell survival and proliferation.

Therefore, therapeutic modulation of the activity of this gene or its protein product may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel CNS_{—}1 Summary: Ag3120 This panel confirms the expression of this gene at low to moderate levels in the brain in an independent group of individuals. Please see Panel 1.3D for a discussion of the potential utility of this gene in treatment of central nervous system disorders.

NOV38

Expression of NOV38/CG57153-01 was assessed using the primer-probe set Ag3121, described in Table AFA. Results of the RTQ-PCR runs are shown in Tables AFB, AFC, AFD and AFE.

TABLE AFA
Probe Name Ag3121

| Primers | Sequences | Length | Start Position | SEQ ID NO |
|---|---|---|---|---|
| Forward | 5′-tccagggtttatatgccagagt-3′ | 22 | 249 | 371 |
| Probe | TET-5′-cacatgtcatgatgacactggcacaa-3′-TAMRA | 26 | 279 | 372 |
| Reverse | 5′-cagacttctcccagtagttgga-3′ | 22 | 307 | 373 |

TABLE AFB
CNS_neurodegeneration_v1.0

| | Rel. Exp. (%) Ag3121, Run | | Rel. Exp. (%) Ag3121, Run |
|---|---|---|---|
| Tissue Name | 208976899 | Tissue Name | 208976899 |
| AD 1 Hippo | 0.0 | Control (Path) 3 Temporal | 3.5 |
| | | Ctx | |
| AD 2 Hippo | 100.0 | Control (Path) 4 Temporal | 33.2 |
| | | Ctx | |
| AD 3 Hippo | 0.0 | AD 1 Occipital Ctx | 13.4 |
| AD 4 Hippo | 15.4 | AD 2 Occipital Ctx | 0.0 |
| | | (Missing) | |
| AD 5 Hippo | 5.0 | AD 3 Occipital Ctx | 0.0 |
| AD 6 Hippo | 66.4 | AD 4 Occipital Ctx | 7.7 |
| Control 2 Hippo | 32.5 | AD 5 Occipital Ctx | 3.8 |
| Control 4 Hippo | 17.8 | AD 6 Occipital Ctx | 3.1 |
| Control (Path) 3 Hippo | 0.0 | Control 1 Occipital Ctx | 0.0 |
| AD 1 Temporal Ctx | 0.0 | Control 2 Occipital Ctx | 0.0 |
| AD 2 Temporal Ctx | 18.0 | Control 3 Occipital Ctx | 0.0 |
| AD 3 Temporal Ctx | 0.0 | Control 4 Occipital Ctx | 0.0 |
| AD 4 Temporal Ctx | 14.0 | Control (Path) 1 Occipital | 6.5 |
| | | Ctx | |
| AD 5 Inf Temporal Ctx | 28.3 | Control (Path) 2 Occipital | 12.7 |
| | | Ctx | |
| AD 5 Sup Temporal Ctx | 37.4 | Control (Path) 3 Occipital | 0.0 |
| | | Ctx | |
| AD 6 Inf Temporal Ctx | 10.8 | Control (Path) 4 Occipital | 0.0 |
| | | Ctx | |
| AD 6 Sup Temporal Ctx | 35.1 | Control 1 Parietal Ctx | 0.0 |
| Control 1 Temporal Ctx | 0.0 | Control 2 Parietal Ctx | 35.4 |
| Control 2 Temporal Ctx | 5.2 | Control 3 Parietal Ctx | 6.4 |
| Control 3 Temporal Ctx | 13.2 | Control (Path) 1 Parietal | 14.5 |
| | | Ctx | |
| Control 3 Temporal Ctx | 0.0 | Control (Path) 2 Parietal | 6.4 |
| | | Ctx | |
| Control (Path) 1 Temporal | 19.8 | Control (Path) 3 Parietal | 0.0 |
| Ctx | | Ctx | |
| Control (Path) 2 Temporal | 28.9 | Control (Path) 4 Parietal | 6.5 |
| Ctx | | Ctx | |

TABLE AFC
Panel 1.3D

| | Rel. Exp. (%) Ag3121, Run | | Rel. Exp. (%) Ag3121, Run |
|---|---|---|---|
| Tissue Name | 167985269 | Tissue Name | 167985269 |
| Liver adenocarcinoma | 0.0 | Kidney (fetal) | 9.3 |
| Pancreas | 0.0 | Renal ca. 786-0 | 0.0 |
| Pancreatic ca. CAPAN 2 | 0.0 | Renal ca. A498 | 0.0 |
| Adrenal gland | 3.3 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 100.0 | Liver | 0.0 |
| Brain (whole) | 0.0 | Liver (fetal) | 0.0 |
| Brain (amygdala) | 6.3 | Liver ca. (hepatoblast) | 0.0 |
| | | HepG2 | |
| Brain (cerebellum) | 0.0 | Lung | 0.0 |
| Brain (hippocampus) | 11.3 | Lung (fetal) | 0.0 |
| Brain (substantia nigra) | 3.1 | Lung ca. (small cell) LX-1 | 0.0 |
| Brain (thalamus) | 0.0 | Lung ca. (small cell) NCI- | 0.0 |
| | | H69 | |
| Cerebral Cortex | 0.0 | Lung ca. (s.cell var.) SHP- | 0.0 |
| | | 77 | |
| Spinal cord | 0.0 | Lung ca. (large cell)NCI- | 0.0 |
| | | H460 | |
| glio/astro U87-MG | 0.0 | Lung ca. (non-sm. cell) | 0.0 |
| | | A549 | |
| glio/astro U-118-MG | 0.0 | Lung ca. (non-s.cell) NCI- | 0.0 |
| | | H23 | |
| astrocytoma SW1783 | 0.0 | Lung ca. (non-s.cell) HOP- | 0.0 |
| | | 62 | |
| neuro*; met SK-N-AS | 6.3 | Lung ca. (non-s.cl) NCI- | 0.0 |
| | | H522 | |
| astrocytoma SF-539 | 0.0 | Lung ca. (squam.) SW 900 | 0.0 |
| astrocytoma SNB-75 | 0.0 | Lung ca. (squam.) NCI- | 0.0 |
| | | H596 | |
| glioma SNB-19 | 3.1 | Mammary gland | 0.0 |
| glioma U251 | 4.8 | Breast ca.* (pl.ef) MCF-7 | 0.0 |
| glioma SF-295 | 0.0 | Breast ca.* (pl.ef) MDA- | 0.0 |
| | | MB-231 | |
| Heart (fetal) | 0.0 | Breast ca.* (pl.ef) T47D | 0.0 |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 2.5 |
| Colorectal | 0.0 | Ovarian ca. IGROV-1 | 0.0 |
| Stomach | 0.0 | Ovarian ca.* (ascites) SK- | 0.0 |
| | | OV-3 | |
| Small intestine | 0.0 | Uterus | 0.0 |
| Colon ca. SW480 | 0.0 | Placenta | 0.0 |
| Colon ca.* SW620(SW480 | 0.0 | Prostate | 0.0 |
| met) | | | |
| Colon ca. HT29 | 0.0 | Prostate ca.* (bone met)PC-3 | 0.0 |
| Colon ca. HCT-116 | 0.0 | Testis | 0.0 |
| Colon ca. CaCo-2 | 0.0 | Melanoma Hs688(A).T | 0.0 |
| Colon ca. tissue(ODO3866) | 4.7 | Melanoma* (met) | 0.0 |
| | | Hs688(B).T | |
| Colon ca. HCC-2998 | 0.0 | Melanoma UACC-62 | 0.0 |
| Gastric ca.* (liver met) NCI- | 0.0 | Melanoma M14 | 0.0 |
| N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE AFD
Panel 4D

| | Rel. Exp. (%) Ag3121, Run | | Rel. Exp. (%) Ag3121, Run |
|---|---|---|---|
| Tissue Name | 164526517 | Tissue Name | 164526517 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 9.7 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + | 0.0 |
| | | IL-1beta | |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 0.0 | Bronchial epithelium TNF alpha + | 33.0 |
| | | IL1beta | |
| Primary Th2 rest | 0.0 | Small airway epithelium none | 0.0 |
| Primary Tr1 rest | 0.0 | Small airway epithelium TNF alpha + | 0.0 |
| | | IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNF alpha + | 0.0 |
| act | | IL-1beta | |
| CD8 lymphocyte act | 0.0 | Astrocytes rest | 0.0 |
| Secondary CD8 lymphocyte | 0.0 | Astrocytes TNF alpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 0.0 | Lung fibroblast TNF alpha + IL- | 10.9 |
| | | 1beta | |
| PBMC PHA-L | 0.0 | Lung fibroblast IL-4 | 22.2 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 rest | 0.0 |
| IL-4 | | | |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL- | 0.0 |
| PMA/ionomycin | | 1beta | |
| Dendritic cells none | 0.0 | Dermal fibroblast IFN gamma | 0.0 |
| Dendritic cells LPS | 0.0 | Dermal fibroblast IL-4 | 37.6 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 28.9 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

TABLE AFE
Panel CNS_1

| | Rel. Exp. (%) | Rel. Exp. (%) | | Rel. Exp. (%) | Rel. Exp. (%) |
|---|---|---|---|---|---|
| | Ag3121, Run | Ag3121, Run | | Ag3121, Run | Ag3121, Run |
| Tissue Name | 171694170 | 182257570 | Tissue Name | 171694170 | 182257570 |
| BA4 Control | 24.0 | 57.0 | BA17 PSP | 0.0 | 0.0 |
| BA4 Control2 | 49.0 | 0.0 | BA17 PSP2 | 0.0 | 0.0 |
| BA4 | 0.0 | 33.2 | Sub Nigra Control | 0.0 | 25.0 |
| Alzheimer's2 | | | | | |
| BA4 | 33.0 | 72.7 | Sub Nigra | 0.0 | 0.0 |
| Parkinson's | | | Control2 | | |
| BA4 | 0.0 | 27.9 | Sub Nigra | 27.9 | 0.0 |
| Parkinson's2 | | | Alzheimer's2 | | |
| BA4 | 11.4 | 0.0 | Sub Nigra | 0.0 | 0.0 |
| Huntington's | | | Parkinson's2 | | |
| BA4 | 0.0 | 0.0 | Sub Nigra | 0.0 | 82.9 |
| Huntington's2 | | | Huntington's | | |
| BA4 PSP | 0.0 | 21.8 | Sub Nigra | 0.0 | 0.0 |
| | | | Huntington's2 | | |
| BA4 PSP2 | 26.8 | 0.0 | Sub Nigra PSP2 | 0.0 | 0.0 |
| BA4 | 8.1 | 30.1 | Sub Nigra | 0.0 | 0.0 |
| Depression | | | Depression | | |
| BA4 | 0.0 | 0.0 | Sub Nigra | 0.0 | 0.0 |
| Depression2 | | | Depression2 | | |
| BA7 Control | 0.0 | 0.0 | Glob Palladus | 0.0 | 0.0 |
| | | | Control | | |
| BA7 Control2 | 29.3 | 0.0 | Glob Palladus | 0.0 | 0.0 |
| | | | Control2 | | |
| BA7 | 0.0 | 0.0 | Glob Palladus | 24.8 | 0.0 |
| Alzheimer's2 | | | Alzheimer's | | |
| BA7 | 0.0 | 0.0 | Glob Palladus | 0.0 | 0.0 |
| Parkinson's | | | Alzheimer's2 | | |
| BA7 | 0.0 | 0.0 | Glob Palladus | 82.4 | 58.2 |
| Parkinson's2 | | | Parkinson's | | |
| BA7 | 29.7 | 19.9 | Glob Palladus | 27.4 | 0.0 |
| Huntington's | | | Parkinson's2 | | |
| BA7 | 28.5 | 0.0 | Glob Palladus PSP | 0.0 | 24.1 |
| Huntington's2 | | | | | |
| BA7 PSP | 0.0 | 0.0 | Glob Palladus | 0.0 | 0.0 |
| | | | PSP2 | | |
| BA7 PSP2 | 0.0 | 0.0 | Glob Palladus | 0.0 | 26.8 |
| | | | Depression | | |
| BA7 | 0.0 | 0.0 | Temp Pole | 21.3 | 33.0 |
| Depression | | | Control | | |
| BA9 Control | 0.0 | 0.0 | Temp Pole | 27.7 | 0.0 |
| | | | Control2 | | |
| BA9 Control2 | 100.0 | 36.1 | Temp Pole | 0.0 | 0.0 |
| | | | Alzheimer's | | |
| BA9 | 0.0 | 0.0 | Temp Pole | 0.0 | 0.0 |
| Alzheimer's | | | Alzheimer's2 | | |
| BA9 | 24.7 | 0.0 | Temp Pole | 0.0 | 31.6 |
| Alzheimer's2 | | | Parkinson's | | |
| BA9 | 0.0 | 26.2 | Temp Pole | 50.3 | 0.0 |
| Parkinson's | | | Parkinson's2 | | |
| BA9 | 0.0 | 30.6 | Temp Pole | 0.0 | 0.0 |
| Parkinson's2 | | | Huntington's | | |
| BA9 | 0.0 | 0.0 | Temp Pole PSP | 0.0 | 0.0 |
| Huntington's | | | | | |
| BA9 | 0.0 | 29.1 | Temp Pole PSP2 | 0.0 | 0.0 |
| Huntington's2 | | | | | |
| BA9 PSP | 0.0 | 100.0 | Temp Pole | 0.0 | 0.0 |
| | | | Depression2 | | |
| BA9 PSP2 | 0.0 | 0.0 | Cing Gyr Control | 0.0 | 94.6 |
| BA9 | 0.0 | 0.0 | Cing Gyr Control2 | 0.0 | 30.8 |
| Depression | | | | | |
| BA9 | 0.0 | 0.0 | Cing Gyr | 0.0 | 0.0 |
| Depression2 | | | Alzheimer's | | |
| BA17 Control | 0.0 | 37.9 | Cing Gyr | 0.0 | 0.0 |
| | | | Alzheimer's2 | | |
| BA17 Control2 | 45.1 | 0.0 | Cing Gyr | 28.3 | 29.3 |
| | | | Parkinson's | | |
| BA17 | 0.0 | 0.0 | Cing Gyr | 0.0 | 91.4 |
| Alzheimer's2 | | | Parkinson's2 | | |
| BA17 | 0.0 | 27.7 | Cing Gyr | 14.2 | 28.7 |
| Parkinson's | | | Huntington's | | |
| BA17 | 0.0 | 43.8 | Cing Gyr | 90.1 | 41.8 |
| Parkinson's2 | | | Huntington's2 | | |
| BA17 | 24.8 | 0.0 | Cing Gyr PSP | 28.1 | 29.9 |
| Huntington's | | | | | |
| BA17 | 0.0 | 0.0 | Cing Gyr PSP2 | 0.0 | 0.0 |
| Huntington's2 | | | | | |
| BA17 | 0.0 | 0.0 | Cing Gyr | 0.0 | 31.2 |
| Depression | | | Depression | | |
| BA17 | 0.0 | 29.1 | Cing Gyr | 0.0 | 0.0 |
| Depression2 | | | Depression2 | | |

CNS_neurodegeneration_v1.0 Summary: Ag3121 This panel confirms the expression of this gene at low levels in the brain in an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag3121 Significant expression of the CG57153-01 gene is restricted to a sample derived from fetal brain tissue (CT=33.1). Interestingly, expression of this gene is much lower in adult brain (CT=40), suggesting that expression of this gene can be used to distinguish fetal brain from adult brain as well as the other samples on this panel. In addition, the relative overexpression of this gene in fetal brain suggests that the protein product may enhance brain development in the fetus and thus may also act in a regenerative capacity in the adult. Therefore, therapeutic modulation of the cyclophilin encoded by this gene could be useful in treatment of neurodegenerative diseases, such as Alzheimer's disease and Parkinson's disease.

Panel 4D Summary: Ag3121 Significant expression of this gene is detected in a liver cirrhosis sample (CT=33.64). Furthermore, expression of this gene is not detected in normal liver in Panel 1.3D, suggesting that its expression is unique to liver cirrhosis. This gene encodes a putative cyclophilin; therefore, small molecule therapeutics designed against this protein could reduce or inhibit fibrosis that occurs in liver cirrhosis. In addition, antibodies to this protein could also be used for the diagnosis of liver cirrhosis.

Panel CNS_{—}1 Summary: Ag3121 Expression of this gene is low/undetectable (CTs>35) across all of the samples on this panel (data not shown).

NOV39

Expression of NOV39./G57155-01 was assessed using the primer-probe set Ag3122, described in Table AGA. Results of the RTQ-PCR runs are shown in Tables AGB, AGC, AGD and AGE.

TABLE AGA
Probe Name Ag3122

| Primers | Sequences | Length | Start Position | SEQ ID NO |
|---|---|---|---|---|
| Forward | 5′-agcaacgggtcacaattctata-3′ | 22 | 355 | 374 |
| Probe | TET-5′-tcacactgcaagcaactccttatctaga-3′-TAMRA | 28 | 377 | 375 |
| Reverse | 5′-atacccaaaagccacaaatttt-3′ | 22 | 408 | 376 |

TABLE AGB
CNS_neurodegeneration_v1.0

| | Rel. Exp. (%) Ag3122, Run | | Rel. Exp. (%) Ag3122, Run |
|---|---|---|---|
| Tissue Name | 208976900 | Tissue Name | 208976900 |
| AD 1 Hippo | 30.4 | Control (Path) 3 Temporal | 9.7 |
| | | Ctx | |
| AD 2 Hippo | 33.4 | Control (Path) 4 Temporal | 28.5 |
| | | Ctx | |
| AD 3 Hippo | 22.1 | AD 1 Occipital Ctx | 12.2 |
| AD 4 Hippo | 10.2 | AD 2 Occipital Ctx | 0.0 |
| | | (Missing) | |
| AD 5 hippo | 100.0 | AD 3 Occipital Ctx | 11.7 |
| AD 6 Hippo | 59.5 | AD 4 Occipital Ctx | 16.2 |
| Control 2 Hippo | 33.4 | AD 5 Occipital Ctx | 22.5 |
| Control 4 Hippo | 27.9 | AD 6 Occipital Ctx | 48.6 |
| Control (Path) 3 Hippo | 14.9 | Control 1 Occipital Ctx | 3.1 |
| AD 1 Temporal Ctx | 23.0 | Control 2 Occipital Ctx | 42.6 |
| AD 2 Temporal Ctx | 0.0 | Control 3 Occipital Ctx | 17.6 |
| AD 3 Temporal Ctx | 10.4 | Control 4 Occipital Ctx | 12.6 |
| AD 4 Temporal Ctx | 22.5 | Control (Path) 1 Occipital | 83.5 |
| | | Ctx | |
| AD 5 Inf Temporal Ctx | 86.5 | Control (Path) 2 Occipital | 13.4 |
| | | Ctx | |
| AD 5 SupTemporal Ctx | 57.8 | Control (Path) 3 Occipital | 4.9 |
| | | Ctx | |
| AD 6 Inf Temporal Ctx | 55.1 | Control (Path) 4 Occipital | 12.2 |
| | | Ctx | |
| AD 6 Sup Temporal Ctx | 80.7 | Control 1 Parietal Ctx | 6.5 |
| Control 1 Temporal Ctx | 5.9 | Control 2 Parietal Ctx | 45.7 |
| Control 2 Temporal Ctx | 43.8 | Control 3 Parietal Ctx | 22.5 |
| Control 3 Temporal Ctx | 14.0 | Control (Path) 1 Parietal | 64.6 |
| | | Ctx | |
| Control 4 Temporal Ctx | 10.1 | Control (Path) 2 Parietal | 31.9 |
| | | Ctx | |
| Control (Path) 1 Temporal | 73.2 | Control (Path) 3 Parietal | 11.0 |
| Ctx | | Ctx | |
| Control (Path) 2 Temporal | 33.7 | Control (Path) 4 Parietal | 48.3 |
| Ctx | | Ctx | |

TABLE AGC
Panel 1.3D

| | Rel. Exp. (%) Ag3122, Run | | Rel. Exp. (%) Ag3122, Run |
|---|---|---|---|
| Tissue Name | 167985270 | Tissue Name | 167985270 |
| Liver adenocarcinoma | 5.7 | Kidney (fetal) | 100.0 |
| Pancreas | 13.2 | Renal ca. 786-0 | 20.6 |
| Pancreatic ca. CAPAN 2 | 0.0 | Renal ca. A498 | 22.5 |
| Adrenal gland | 0.0 | Renal ca. RXF 393 | 22.1 |
| Thyroid | 40.1 | Renal ca. ACHN | 28.3 |
| Salivary gland | 0.0 | Renal ca. UO-31 | 5.0 |
| Pituitary gland | 19.1 | Renal ca. TK-10 | 9.5 |
| Brain (fetal) | 88.9 | Liver | 0.0 |
| Brain (whole) | 11.0 | Liver (fetal) | 0.0 |
| Brain (amygdala) | 73.7 | Liver ca. (hepatoblast) | 7.2 |
| | | HepG2 | |
| Brain (cerebellum) | 45.7 | Lung | 8.5 |
| Brain (hippocampus) | 33.0 | Lung (fetal) | 77.4 |
| Brain (substantia nigra) | 27.7 | Lung ca. (small cell) LX-1 | 23.5 |
| Brain (thalamus) | 11.5 | Lung ca. (small cell) NCI- | 20.6 |
| | | H69 | |
| Cerebral Cortex | 12.2 | Lung ca. (s.cell var.) SHP- | 56.6 |
| | | 77 | |
| Spinal cord | 55.9 | Lung ca. (large cell)NCI- | 0.0 |
| | | H460 | |
| glio/astro U87-MG | 9.7 | Lung ca. (non-sm. cell) | 34.9 |
| | | A549 | |
| glio/astro U-118-MG | 2.5 | Lung ca. (non-s.cell) NCI- | 40.3 |
| | | H23 | |
| astrocytoma SW1783 | 3.5 | Lung ca. (non-s.cell) HOP- | 8.4 |
| | | 62 | |
| neuro*; met SK-N-AS | 6.1 | Lung ca. (non-s.cl) NCI- | 33.2 |
| | | H522 | |
| astrocytoma SF-539 | 5.3 | Lung ca. (squam.) SW 900 | 14.9 |
| astrocytoma SNB-75 | 12.2 | Lung ca. (squam.) NCI- | 39.8 |
| | | H596 | |
| glioma SNB-19 | 11.3 | Mammary gland | 0.0 |
| glioma U251 | 13.1 | Breast ca.* (pl.ef) MCF-7 | 0.0 |
| glioma SF-295 | 36.1 | Breast ca.* (pl.ef) MDA- | 6.9 |
| | | MB-231 | |
| Heart (fetal) | 20.6 | Breast ca.* (pl.ef) T47D | 77.9 |
| Heart | 0.0 | Breast ca. BT-549 | 3.7 |
| Skeletal muscle (fetal) | 14.5 | Breast ca. MDA-N | 2.8 |
| Skeletal muscle | 22.2 | Ovary | 13.0 |
| Bone marrow | 0.0 | Ovarian ca. OVCAR-3 | 12.0 |
| Thymus | 12.5 | Ovarian ca. OVCAR-4 | 6.9 |
| Spleen | 3.6 | Ovarian ca. OVCAR-5 | 29.3 |
| Lymph node | 17.8 | Ovarian ca. OVCAR-8 | 2.7 |
| Colorectal | 4.9 | Ovarian ca. IGROV-1 | 0.0 |
| Stomach | 0.0 | Ovarian ca.* (ascites) SK- | 39.8 |
| | | OV-3 | |
| Small intestine | 6.2 | Uterus | 18.4 |
| Colon ca. SW480 | 0.0 | Placenta | 0.0 |
| Colon ca.* SW620(SW480 | 13.0 | Prostate | 4.1 |
| met) | | | |
| Colon ca. HT29 | 5.1 | Prostate ca.* (bone met)PC-3 | 8.4 |
| Colon ca. HCT-116 | 0.0 | Testis | 73.7 |
| Colon ca. CaCo-2 | 0.0 | Melanoma Hs688(A).T | 0.0 |
| Colon ca. tissue(ODO3866) | 17.3 | Melanoma* (met) | 0.0 |
| | | Hs688(B).T | |
| Colon ca. HCC-2998 | 25.7 | Melanoma UACC-62 | 2.1 |
| Gastric ca.* (liver met) NCI- | 35.4 | Melanoma M14 | 5.6 |
| N87 | | | |
| Bladder | 14.2 | Melanoma LOX IMVI | 0.0 |
| Trachea | 57.4 | Melanoma* (met) SK- | 0.0 |
| | | MEL-5 | |
| Kidney | 59.0 | Adipose | 3.6 |

TABLE AGD
Panel 4D

| | Rel. Exp. (%) Ag3122, Run | | Rel. Exp. (%) Ag3122, Run |
|---|---|---|---|
| Tissue Name | 164527262 | Tissue Name | 164527262 |
| Secondary Th1 act | 0.0 | HUVEC IL-1beta | 0.0 |
| Secondary Th2 act | 1.4 | HUVEC IFN gamma | 3.1 |
| Secondary Tr1 act | 0.0 | HUVEC TNF alpha + IFN gamma | 3.8 |
| Secondary Th1 rest | 0.0 | HUVEC TNF alpha + IL4 | 1.1 |
| Secondary Th2 rest | 0.0 | HUVEC IL-11 | 0.0 |
| Secondary Tr1 rest | 0.0 | Lung Microvascular EC none | 3.2 |
| Primary Th1 act | 1.6 | Lung Microvascular EC TNF alpha + | 2.2 |
| | | IL-1beta | |
| Primary Th2 act | 3.1 | Microvascular Dermal EC none | 0.4 |
| Primary Tr1 act | 0.0 | Microsvasular Dermal EC | 1.6 |
| | | TNF alpha + IL-1beta | |
| Primary Th1 rest | 1.4 | Bronchial epithelium TNF alpha + | 10.4 |
| | | IL1beta | |
| Primary Th2 rest | 0.0 | Small airway epithelium none | 2.1 |
| Primary Tr1 rest | 0.0 | Small airway epithelium TNF alpha + | 7.4 |
| | | IL-1beta | |
| CD45RA CD4 lymphocyte | 0.5 | Coronery artery SMC rest | 1.5 |
| act | | | |
| CD45RO CD4 lymphocyte | 0.0 | Coronery artery SMC TNF alpha + | 0.8 |
| act | | IL-1beta | |
| CD8 lymphocyte act | 1.9 | Astrocytes rest | 6.3 |
| Secondary CD8 lymphocyte | 3.3 | Astrocytes TNF alpha + IL-1beta | 3.3 |
| rest | | | |
| Secondary CD8 lymphocyte | 1.8 | KU-812 (Basophil) rest | 7.9 |
| act | | | |
| CD4 lymphocyte none | 0.0 | KU-812 (Basophil) | 15.5 |
| | | PMA/ionomycin | |
| 2ry Th1/Th2/Tr1_anti-CD95 | 1.5 | CCD1106 (Keratinocytes) none | 1.4 |
| CH11 | | | |
| LAK cells rest | 0.8 | CCD1106 (Keratinocytes) | 0.0 |
| | | TNF alpha + IL-1beta | |
| LAK cells IL-2 | 0.6 | Liver cirrhosis | 2.9 |
| LAK cells IL-2 + IL-12 | 4.5 | Lupus kidney | 2.2 |
| LAK cells IL-2 + IFN gamma | 1.9 | NCI-H292 none | 4.1 |
| LAK cells IL-2 + IL-18 | 2.0 | NCI-H292 IL-4 | 4.8 |
| LAK cells PMA/ionomycin | 0.0 | NCI-H292 IL-9 | 9.9 |
| NK Cells IL-2 rest | 100.0 | NCI-H292 IL-13 | 4.5 |
| Two Way MLR 3 day | 7.7 | NCI-H292 IFN gamma | 1.6 |
| Two Way MLR 5 day | 0.0 | HPAEC none | 0.6 |
| Two Way MLR 7 day | 0.0 | HPAEC TNF alpha + IL-1beta | 1.0 |
| PBMC rest | 2.8 | Lung fibroblast none | 3.2 |
| PBMC PWM | 4.5 | Lung fibroblast TNF alpha + IL- | 1.4 |
| | | 1beta | |
| PBMC PHA-L | 0.9 | Lung fibroblast IL-4 | 5.8 |
| Ramos (B cell) none | 0.0 | Lung fibroblast IL-9 | 1.1 |
| Ramos (B cell) ionomycin | 8.0 | Lung fibroblast IL-13 | 3.8 |
| B lymphocytes PWM | 9.2 | Lung fibroblast IFN gamma | 13.8 |
| B lymphocytes CD40L and | 2.5 | Dermal fibroblast CCD 1070 rest | 1.2 |
| IL-4 | | | |
| EOL-1 dbcAMP | 0.0 | Dermal fibroblast CCD1070 TNF | 3.2 |
| | | alpha | |
| EOL-1 dbcAMP | 3.0 | Dermal fibroblast CCD1070 IL- | 2.5 |
| PMA/ionomycin | | 1beta | |
| Dendritic cells none | 1.0 | Dermal fibroblast IFN gamma | 1.2 |
| Dendritic cells LPS | 2.5 | Dermal fibroblast IL-4 | 5.8 |
| Dendritic cells anti-CD40 | 0.7 | IBD Colitis 2 | 1.4 |
| Monocytes rest | 1.6 | IBD Crohn's | 3.2 |
| Monocytes LPS | 1.4 | Colon | 4.8 |
| Macrophages rest | 2.9 | Lung | 11.9 |
| Macrophages LPS | 0.0 | Thymus | 59.9 |
| HUVEC none | 0.0 | Kidney | 6.1 |
| HUVEC starved | 1.3 | | |

TABLE AGE
Panel CNS_1

| | Rel. Exp. (%) Ag3122, Run | | Rel. Exp. (%) Ag3122, Run |
|---|---|---|---|
| Tissue Name | 182257573 | Tissue Name | 182257573 |
| BA4 Control | 47.0 | BA17 PSP | 19.1 |
| BA4 Control2 | 81.2 | BA17 PSP2 | 9.2 |
| BA4 Alzheimer's2 | 0.0 | Sub Nigra Control | 27.7 |
| BA4 Parkinson's | 53.6 | Sub Nigra Control2 | 14.2 |
| BA4 Parkinson's2 | 72.7 | Sub Nigra Alzheimer's2 | 16.4 |
| BA4 Huntington's | 47.0 | Sub Nigra Parkinson's2 | 24.0 |
| BA4 Huntington's2 | 8.2 | Sub Nigra Huntington's2 | 100.0 |
| BA4 PSP | 7.7 | Sub Nigra Huntington's2 | 36.1 |
| BA4 PSP2 | 7.5 | Sub Nigra PSP2 | 15.9 |
| BA4 Depression | 24.3 | Sub Nigra Depression | 16.3 |
| BA4 Depression2 | 15.3 | Sub Nigra Depression2 | 0.0 |
| BA7 Control | 29.5 | Glob Palladus Control | 18.2 |
| BA7 Control2 | 24.5 | Glob Palladus Control2 | 99.3 |
| BA7 Alzheimer's2 | 24.0 | Glob Palladus Alzheimer's | 0.0 |
| BA7 Parkinson's | 20.9 | Glob Palladus | 8.7 |
| | | Alzheimer's2 | |
| BA7 Parkinson's2 | 29.3 | Glob Palladus Parkinson's | 100.0 |
| BA7 Huntington's | 31.9 | Glob Palladus | 15.8 |
| | | Parkinson's2 | |
| BA7 Huntington's2 | 68.8 | Glob Palladus PSP | 7.2 |
| BA7 PSP | 11.0 | Glob Palladus PSP2 | 0.0 |
| BA7 PSP2 | 7.5 | Glob Palladus Depression | 9.7 |
| BA7 Depression | 11.3 | Temp Pole Control | 12.3 |
| BA9 Control | 24.3 | Temp Pole Control2 | 32.5 |
| BA9 Control2 | 44.4 | Temp Pole Alzheimer's | 11.9 |
| BA9 Alzheimer's | 8.8 | Temp Pole Alzheimer's2 | 0.0 |
| BA9 Alzheimer's2 | 19.3 | Temp Pole Parkinson's | 37.1 |
| BA9 Parkinson's | 28.5 | Temp Pole Parkinson's2 | 30.1 |
| BA9 Parkinson's2 | 30.4 | Temp Pole Huntington's | 28.1 |
| BA9 Huntington's | 54.7 | Temp Pole PSP | 14.7 |
| BA9 Huntington's2 | 17.4 | Temp Pole PSP2 | 0.0 |
| BA9 PSP | 0.0 | Temp Pole Depression2 | 19.8 |
| BA9 PSP2 | 7.4 | Cing Gyr Control | 49.0 |
| BA9 Depression | 12.6 | Cing Gyr Control2 | 19.3 |
| BA9 Depression2 | 0.0 | Cing Gyr Alzheimer's | 11.0 |
| BA17 Control | 50.7 | Cing Gyr Alzheimer's2 | 6.3 |
| BA17 Control2 | 35.8 | Cing Gyr Parkinson's | 39.8 |
| BA17 Alzheimer's2 | 9.0 | Cing Gyr Parkinson's2 | 31.6 |
| BA17 Parkinson's | 38.4 | Cing Gyr Huntington's | 59.5 |
| BA17 Parkinson's2 | 60.7 | Cing Gyr Huntington's2 | 13.5 |
| BA17 Huntington's | 19.6 | Cing Gyr PSP | 26.4 |
| BA17 | 34.4 | Cing Gyr PSP2 | 0.0 |
| Huntington's2 | | | |
| BA17 Depression | 14.4 | Cing Gyr Depression | 0.0 |
| BA17 Depression2 | 24.0 | Cing Gyr Depression2 | 12.5 |

CNS_neurodegeneration_v1.0 Summary: Ag3122 This panel confirms the expression of this gene at low levels in the brain in an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag3122 Expression of the CG57155-01 gene is highest in fetal kidney (CT=34.3). In addition, low but significant expression of this gene is detected in fetal brain (CT=34.5) and fetal lung (CT=34.6). Interestingly, expression of the CG57155-01 gene is much lower in adult brain (CT=37.5) and adult lung (CT=37.8). This observation suggests that expression of this gene can be used to distinguish fetal brain from adult brain as well as fetal lung from adult lung. In addition, the relative overexpression of this gene in fetal brain suggests that the protein product may enhance brain development in the fetus and thus may also act in a regenerative capacity in the adult. Therefore, therapeutic modulation of the cyclophilin encoded by this gene could be useful in treatment of neurodegenerative diseases.

Panel 4D Summary: Ag3122 Expression of the CG57155-01 gene is highest in natural killer cells (CT=31). This observation suggests that therapeutic modulation of this gene using small molecule drugs could be of use in the treatment of viral or bacterial intracellular infections.

In addition, this gene is expressed at significant levels in thymus (CT=31.8). The putative cyclophilin encoded for by the CG57155-01 gene could therefore play an important role in T cell development. Small molecule therapeutics, or antibody therapeutics designed against the protein encoded for by this gene could be utilized to modulate immune function (T cell development) and be important for organ transplant, AIDS treatment or post chemotherapy immune reconstitiution.

This gene encodes a protein with homology to peptidyl-prolyl cis/trans isomerase A (PPIA or cyclophilin A), a cytosolic enzyme involved in protein folding and/or intracellular protein transport. Cyclophilin genes have been shown to be expressed in numerous tissues. The interaction of cyclophilin and cyclosporine has an effect on cytokine production in T-cells. Therefore, modulation of this gene may be used in the treatment of inflamatory diseases such as rheumatoid arthritis.

References:

1. Russell G, Graveley R, Seid J, al-Humidan A K, Skjodt H. Mechanisms of action of cyclosporine and effects on connective tissues. Semin Arthritis Rheum 1992 June;21(6 Suppl 3):16-22.

Cyclosporine is a potent immunomodulatory agent with an increasing number of clinical applications. Its major mode of action is inhibition of the production of cytokines involved in the regulation of T-cell activation. In particular, cyclosporine inhibits the transcription of interleukin 2. Although cyclosporine's major actions are on T cells, there is some evidence that it produces direct effects on other cell types. Its immunosuppressive action is closely linked to its binding of cyclophilin, a member of a family of high-affinity cyclosporine-binding proteins widely distributed in different cell types and in different species. The cyclophilins have been shown to have peptidyl-prolyl cis-trans isomerase enzyme activity that is blocked by cyclosporine. Although this may be a factor in cyclosporine's selective inhibition of cytokine gene transcription, it is still unclear whether inhibition of this activity is the mechanism through which cyclosporine exerts its effects on target cells. The ubiquitous presence of cyclophilins raises the question of why cyclosporine has major effects on T cells. Perhaps the critical proteins affected are transcriptional regulators restricted in their tissue distribution. The effects of cyclosporine on T cells and, directly or indirectly, on connective tissue cells, all of which can produce a range of cytokines, are of interest in relation to the tissue changes that occur in such inflammatory conditions as rheumatoid arthritis.

PMID: 1502562

Panel CNS_{—}1 Summary: Ag3122 This panel confirms the expression of this gene at low levels in the brain in an independent group of individuals. Please see Panel 1.3D for a discussion of the potential utility of this gene in treatment of central nervous system disorders.

NOV40

Expression of NOV40/CG57157-01 was assessed using the primer-probe set Ag3123, described in Table AHA. Results of the RTQ-PCR runs are shown in Tables AHB.

TABLE AHA
Probe Name Ag3123

| Primers | Sequences | Length | Start Position | SEQ ID NO |
|---|---|---|---|---|
| Forward | 5′-gtgagactggcaagaagatcac-3′ | 22 | 497 | 377 |
| Probe | TET-5′-ctgccaactgcggacaactctaatca-3′-TAMRA | 26 | 521 | 378 |
| Reverse | 5′-tggttaagatcaaacacaagca-3′ | 22 | 550 | 379 |

TABLE AHB
Panel 4D

| | Rel. Exp. (%) Ag3123, Run | | Rel. Exp. (%) Ag3123, Run |
|---|---|---|---|
| Tissue Name | 164527712 | Tissue Name | 164527712 |
| Secondary Th1 act | 9.3 | HUVEC IL-1beta | 9.4 |
| Secondary Th2 act | 1.5 | HUVEC IFN gamma | 25.7 |
| Secondary Tr1 act | 10.2 | HUVEC TNF alpha + IFN gamma | 0.9 |
| Secondary Th1 rest | 1.1 | HUVEC TNF alpha + IL4 | 18.0 |
| Secondary Th2 rest | 3.5 | HUVEC IL-11 | 52.1 |
| Secondary Tr1 rest | 1.4 | Lung Microvascular EC none | 28.9 |
| Primary Th1 act | 15.3 | Lung Microvascular EC TNF alpha + | 18.9 |
| | | IL-1beta | |
| Primary Th2 act | 6.5 | Microvascular Dermal EC none | 50.3 |
| Primary Tr1 act | 30.8 | Microsvasular Dermal EC | 18.8 |
| | | TNF alpha + IL-1beta | |
| Primary Th1 rest | 23.0 | Bronchial epithelium TNF alpha + | 11.7 |
| | | IL1beta | |
| Primary Th2 rest | 9.7 | Small airway epithelium none | 4.7 |
| Primary Tr1 rest | 5.4 | Small airway epithelium TNF alpha + | 27.7 |
| | | IL-1beta | |
| CD45RA CD4 lymphocyte | 2.4 | Coronery artery SMC rest | 15.9 |
| act | | | |
| CD45RO CD4 lymphocyte | 5.9 | Coronery artery SMC TNF alpha + | 8.2 |
| act | | IL-1beta | |
| CD8 lymphocyte act | 1.1 | Astrocytes rest | 38.2 |
| Secondary CD8 lymphocyte | 4.9 | Astrocytes TNF alpha + IL-1beta | 8.0 |
| rest | | | |
| Secondary CD8 lymphocyte | 3.5 | KU-812 (Basophil) rest | 57.0 |
| act | | | |
| CD4 lymphocyte none | 0.0 | KU-812 (Basophil) | 89.5 |
| | | PMA/ionomycin | |
| 2ry Th1/Th2/Tr1_anti-CD95 | 4.8 | CCD1106 (Keratinocytes) none | 24.5 |
| CH11 | | | |
| LAK cells rest | 1.2 | CCD1106 (Keratinocytes) | 14.4 |
| | | TNF alpha + IL-1beta | |
| LAK cells IL-2 | 1.4 | Liver cirrhosis | 19.1 |
| LAK cells IL-2 + IL-12 | 0.9 | Lupus kidney | 10.0 |
| LAK cells IL-2 + IFN gamma | 1.7 | NCI-H292 none | 43.5 |
| LAK cells IL-2 + IL-18 | 1.1 | NCI-H292 IL-4 | 52.9 |
| LAK cells PMA/ionomycin | 0.0 | NCI-H292 IL-9 | 51.8 |
| NK Cells IL-2 rest | 3.6 | NCI-H292 IL-13 | 29.1 |
| Two Way MLR 3 day | 0.0 | NCI-H292 IFN gamma | 19.2 |
| Two Way MLR 5 day | 0.0 | HPAEC none | 24.5 |
| Two Way MLR 7 day | 4.8 | HPAEC TNF alpha + IL-1beta | 3.5 |
| PBMC rest | 0.0 | Lung fibroblast none | 4.6 |
| PBMC PWM | 10.4 | Lung fibroblast TNF alpha + IL- | 1.2 |
| | | 1beta | |
| PBMC PHA-L | 26.2 | Lung fibroblast IL-4 | 7.0 |
| Ramos (B cell) none | 14.0 | Lung fibroblast IL-9 | 21.6 |
| Ramos (B cell) ionomycin | 34.9 | Lung fibroblast IL-13 | 5.9 |
| B lymphocytes PWM | 30.1 | Lung fibroblast IFN gamma | 3.3 |
| B lymphocytes CD40L and | 12.8 | Dermal fibroblast CCD1070 rest | 38.4 |
| IL-4 | | | |
| EOL-1 dbcAMP | 3.5 | Dermal fibroblast CCD1070 TNF | 63.3 |
| | | alpha | |
| EOL-1 dbcAMP | 0.9 | Dermal fibroblast CCD1070 IL- | 15.9 |
| PMA/ionomycin | | 1beta | |
| Dendritic cells none | 2.1 | Dermal fibroblast IFN gamma | 10.4 |
| Dendritic cells LPS | 1.0 | Dermal fibroblast IL-4 | 8.4 |
| Dendritic cells anti-CD40 | 0.0 | IBD Colitis 2 | 1.7 |
| Monocytes rest | 0.0 | IBD Crohn's | 1.9 |
| Monocytes LPS | 1.3 | Colon | 7.1 |
| Macrophages rest | 4.6 | Lung | 17.6 |
| Macrophages LPS | 0.0 | Thymus | 61.6 |
| HUVEC none | 17.8 | Kidney | 30.4 |
| HUVEC starved | 100.0 | | |

Panel 1.3D Summary: Ag3123 Results from one experiment with the CG57157-01 gene are not included. The amp plot indicates that there were experimental difficulties with this run (data not shown).

Panel 4D Summary: Ag3123 The CG57157-01 gene is expressed at low levels in T cells and is also expressed at significant levels in endothelium, epithelium and fibroblasts as well as in normal thymus and kidney. Furthermore, treatment of proinflammatory mediators reduces the level of this transcript in HUVEC cells as compared to in starved cells. The protein encoded for by this transcript may be important in the normal function of the cell types that express it. Regulation of the protein encoded for by this transcript could be important for maintaining or resuming normal homeostasis.

NOV41

Expression of NOV41/CG57159-01 was assessed using the primer-probe set Ag3320, described in Table AIA. Results of the RTQ-PCR runs are shown in Tables AIB and AIC.

TABLE AIA
Probe Name Ag3320

| Primers | Sequences | Length | Start Position | SEQ ID NO |
|---|---|---|---|---|
| Forward | 5′-ttggtgataaatgtccctgc-3′ | 20 | 161 | 380 |
| Probe | TET-5′-ggggtgtgtcagggtggtgacttc-3′-TAMRA | 24 | 199 | 381 |
| Reverse | 5′-ccagtgccattatggtgtgt-3′ | 20 | 223 | 382 |

TABLE AIB
General_screening_panel_v1.4

| | Rel. Exp. (%) Ag3320, Run | | Rel. Exp. (%) Ag3320, Run |
|---|---|---|---|
| Tissue Name | 215775833 | Tissue Name | 215775833 |
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI- | 0.0 |
| | | N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell carcinoma | 0.0 | Colon ca.* (SW480 met) | 0.0 |
| SCC-4 | | SW620 | |
| Testis Pool | 0.0 | Colon ca. HT29 | 0.0 |
| Prostate ca.* (bone met) | 0.0 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 0.0 | Small Intestine Pool | 0.0 |
| Ovarian ca. IGROV-1 | 0.0 | Stomach Pool | 0.1 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.3 |
| Breast ca. MDA-MB-231 | 0.0 | Lymph Node Pool | 0.2 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer (glio/astro) U87- | 0.0 |
| | | MG | |
| Lung | 0.6 | CNS cancer (glio/astro) U- | 0.0 |
| | | 118-MG | |
| Fetal Lung | 0.0 | CNS cancer (neuro; met) SK- | 0.0 |
| | | N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.1 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) Pool | 0.0 |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 1.6 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.1 |
| Renal ca. ACHN | 0.0 | Pancreatic ca. CAPAN2 | 0.0 |
| Renal ca. UO-31 | 0.0 | Pancreas Pool | 100.0 |

CNS_neurodegeneration_v1.0 Summary: Ag3320 Expression of this gene is low/undetectable (CTs>35) across all of the samples on this panel (data not shown).

General_screening_panel_v1.4 Summary: Ag3320 Expression of the CG57159-01 gene is restricted to a sample derived from pancreatic tissue (CT=27.6). Thus, expression of this gene could be used to distinguish pancreas from the other samples in the panel. In addition, therapeutic modulation of the activity of this gene or its protein product may prove useful in the treatment of metabolically related diseases, such as obesity and diabetes.

Panel 4D Summary: Ag3320 Expression of this gene is low/undetectable (CTs>35) across all of the samples on this panel (data not shown).

NOV42

Expression of gene NOV42A/CG57226-01 and NOV42B/CG57226-02 was assessed using the primer-probe set Ag3142, described in Table AJA. Results of the RTQ-PCR runs are shown in Tables AJB, AJC and AJD. Please note that CG57226-02 represents a full-length physical clone of the CG57226-01 gene, validating the prediction of the gene sequence.

TABLE AJA
Probe Name Ag3142

| | | | Start | |
|---|---|---|---|---|
| Primers | Sequences | Length | Position | SEQ ID NO |
| Forward | 5′-caaaacaaatgagtcccagttt-3′ | 22 | 504 | 383 |
| Probe | TET-5′-atctgcactgccatggccaaatg-3′-TAMRA | 23 | 529 | 384 |
| Reverse | 5′-ctgccaaagatcacatgctt-3′ | 20 | 562 | 385 |

TABLE AJB
CNS_neurodegeneration_v1.0

| | Rel. Exp. (%) | | Rel. Exp. (%) |
|---|---|---|---|
| Tissue Name | Ag3142, Run 209055796 | Tissue Name | Ag3142, Run 209055796 |
| AD 1 Hippo | 14.4 | Control (Path) 3 | 3.4 |
| | | Temporal Ctx | |
| AD 2 Hippo | 25.0 | Control (Path) 4 | 71.7 |
| | | Temporal Ctx | |
| AD 3 Hippo | 10.8 | AD 1 Occipital | 27.9 |
| | | Ctx | |
| AD 4 Hippo | 19.8 | AD 2 Occipital | 0.0 |
| | | Ctx (Missing) | |
| AD 5 hippo | 82.9 | AD 3 Occipital | 13.1 |
| | | Ctx | |
| AD 6 Hippo | 58.2 | AD 4 Occipital | 29.5 |
| | | Ctx | |
| Control 2 Hippo | 30.1 | AD 5 Occipital | 15.0 |
| | | Ctx | |
| Control 4 Hippo | 12.2 | AD 6 Occipital | 31.0 |
| | | Ctx | |
| Control (Path) 3 | 9.0 | Control 1 Occipital | 7.1 |
| Hippo | | Ctx | |
| AD 1 Temporal | 49.0 | Control 2 Occipital | 32.5 |
| Ctx | | Ctx | |
| AD 2 Temporal | 28.5 | Control 3 Occipital | 0.0 |
| Ctx | | Ctx | |
| AD 3 Temporal | 11.3 | Control 4 Occipital | 11.4 |
| Ctx | | Ctx | |
| AD 4 Temporal | 47.0 | Control (Path) 1 | 100.0 |
| Ctx | | Occipital Ctx | |
| AD 5 Inf | 89.5 | Control (Path) 2 | 26.1 |
| Temporal Ctx | | Occipital Ctx | |
| AD 5 | 49.7 | Control (Path) 3 | 1.8 |
| SupTemporal Ctx | | Occipital Ctx | |
| AD 6 Inf | 54.3 | Control (Path) 4 | 36.3 |
| Temporal Ctx | | Occipital Ctx | |
| AD 6 Sup | 69.3 | Control 1 Parietal | 9.0 |
| Temporal Ctx | | Ctx | |
| Control 1 | 11.5 | Control 2 Parietal | 57.0 |
| Temporal Ctx | | Ctx | |
| Control 2 | 20.3 | Control 3 Parietal | 13.6 |
| Temporal Ctx | | Ctx | |
| Control 3 | 21.3 | Control (Path) 1 | 54.7 |
| Temporal Ctx | | Parietal Ctx | |
| Control 4 | 11.0 | Control (Path) 2 | 34.4 |
| Temporal Ctx | | Parietal Ctx | |
| Control (Path) 1 | 66.0 | Control (Path) 3 | 9.0 |
| Temporal Ctx | | Parietal Ctx | |
| Control (Path) 2 | 55.1 | Control (Path) 4 | 65.5 |
| Temporal Ctx | | Parietal Ctx | |

TABLE AJC
Panel 1.3D

| | Rel. Exp. (%) | | Rel. Exp. (%) |
|---|---|---|---|
| Tissue Name | Ag3142, Run 167994809 | Tissue Name | Ag3142, Run 167994809 |
| Liver | 16.6 | Kidney (fetal) | 20.2 |
| adenocarcinoma | | | |
| Pancreas | 6.8 | Renal ca. 786-0 | 6.6 |
| Pancreatic ca. | 5.7 | Renal ca. A498 | 8.7 |
| CAPAN 2 | | | |
| Adrenal gland | 3.6 | Renal ca. RXF 393 | 5.7 |
| Thyroid | 2.7 | Renal ca. ACHN | 2.4 |
| Salivary gland | 9.0 | Renal ca. UO-31 | 1.1 |
| Pituitary gland | 9.7 | Renal ca. TK-10 | 18.3 |
| Brain (fetal) | 16.7 | Liver | 11.3 |
| Brain (whole) | 12.8 | Liver (fetal) | 8.3 |
| Brain (amygdala) | 12.2 | Liver ca. | 11.0 |
| | | (hepatoblast) HepG2 | |
| Brain (cerebellum) | 27.0 | Lung | 2.3 |
| Brain (hippocampus) | 13.9 | Lung (fetal) | 10.4 |
| Brain (substania | 16.5 | Lung ca. (small cell) | 16.6 |
| nigra) | | LX-1 | |
| Brain (thalamus) | 4.7 | Lung ca. (small cell) | 14.3 |
| | | NCI-H69 | |
| Cerebral Cortex | 5.1 | Lung ca. (s.cell var.) | 78.5 |
| | | SHP-77 | |
| Spinal cord | 3.3 | Lung ca. (large | 2.5 |
| | | cell)NCI-H460 | |
| glio/astro U87-MG | 13.5 | Lung ca. (non-sm. | 25.0 |
| | | cell) A549 | |
| glio/astro U-118-MG | 11.1 | Lung ca. (non-s.cell) | 12.4 |
| | | NCI-H23 | |
| astrocytoma | 8.8 | Lung ca. (non-s.cell) | 16.0 |
| SW1783 | | HOP-62 | |
| neuro*; met SK-N- | 28.7 | Lung ca. (non-s.cl) | 12.6 |
| AS | | NCI-H522 | |
| astrocytoma SF-539 | 10.1 | Lung ca. (squam.) | 16.5 |
| | | SW 900 | |
| astrocytoma SNB-75 | 13.2 | Lung ca. (squam.) | 48.6 |
| | | NCI-H596 | |
| glioma SNB-19 | 30.4 | Mammary gland | 2.1 |
| glioma U251 | 36.6 | Breast ca.* (pl.ef) | 14.7 |
| | | MCF-7 | |
| glioma SF-295 | 13.6 | Breast ca.* (pl.ef) | 14.1 |
| | | MDA-MB-231 | |
| Heart (fetal) | 0.6 | Breast ca.* (pl.ef) | 57.8 |
| | | T47D | |
| Heart | 8.7 | Breast ca. BT-549 | 4.9 |
| Skeletal muscle | 2.5 | Breast ca. MDA-N | 7.5 |
| (fetal) | | | |
| Skeletal muscle | 2.9 | Ovary | 1.3 |
| Bone marrow | 8.0 | Ovarian ca. | 23.0 |
| | | OVCAR-3 | |
| Thymus | 20.6 | Ovarian ca. | 4.2 |
| | | OVCAR-4 | |
| Spleen | 7.6 | Ovarian ca. | 100.0 |
| | | OVCAR-5 | |
| Lymph node | 26.6 | Ovarian ca. | 4.9 |
| | | OVCAR-8 | |
| Colorectal | 20.3 | Ovarian ca. IGROV-1 | 3.0 |
| Stomach | 9.5 | Ovarian ca.* | 66.4 |
| | | (ascites) SK-OV-3 | |
| Small intestine | 15.6 | Uterus | 7.3 |
| Colon ca. SW480 | 11.4 | Placenta | 0.0 |
| Colon ca.* | 59.5 | Prostate | 4.0 |
| SW620(SW480 met) | | | |
| Colon ca. HT29 | 18.3 | Prostate ca.* (bone | 12.2 |
| | | met)PC-3 | |
| Colon ca. HCT-116 | 14.8 | Testis | 3.1 |
| Colon ca. CaCo-2 | 26.8 | Melonoma | 3.9 |
| | | Hs688(A).T | |
| Colon ca. | 6.9 | Melanoma* (met) | 9.4 |
| tissue(ODO3866) | | Hs688(B).T | |
| Colon ca. HCC-2998 | 28.7 | Melanoma UACC- | 3.2 |
| | | 62 | |
| Gastric ca.* (liver | 25.2 | Melanoma M14 | 2.9 |
| met) NCI-N87 | | | |
| Bladder | 51.4 | Melanoma LOX | 0.0 |
| | | IMVI | |
| Trachea | 2.4 | Melanoma* (met) | 6.6 |
| | | SK-MEL-5 | |
| Kidney | 11.4 | Adipose | 21.8 |

TABLE AJD
Panel 4D

| | Rel. Exp. (%) | | Rel. Exp. (%) |
|---|---|---|---|
| Tissue Name | Ag3142, Run 164527962 | Tissue Name | Ag3142, Run 164527962 |
| Secondary Th1 act | 30.8 | HUVEC IL-1beta | 11.5 |
| Secondary Th2 act | 30.1 | HUVEC IFN gamma | 12.8 |
| Secondary Tr1 act | 34.6 | HUVEC TNF alpha + IFN | 14.1 |
| | | gamma | |
| Secondary Th1 rest | 7.1 | HUVEC TNF alpha + IL4 | 7.6 |
| Secondary Th2 rest | 20.4 | HUVEC IL-11 | 7.7 |
| Secondary Tr1 rest | 21.0 | Lung Microvascular EC | 14.9 |
| | | none | |
| Primary Th1 act | 32.1 | Lung Microvascular EC | 19.6 |
| | | TNF alpha + IL-1beta | |
| Primary Th2 act | 46.3 | Microvascular Dermal EC | 17.9 |
| | | none | |
| Primary Tr1 act | 47.3 | Microvasular Dermal EC | 11.3 |
| | | TNF alpha + IL-1beta | |
| Primary Th1 rest | 76.8 | Bronchial epithelium | 10.4 |
| | | TNF alpha + IL1-beta | |
| Primary Th2 rest | 42.9 | Small airway epithelium | 1.3 |
| | | none | |
| Primary Tr1 rest | 42.3 | Small airway epithelium | 21.3 |
| | | TNF alpha + IL-1beta | |
| CD45RA CD4 | 15.4 | Coronery artery SMC rest | 6.7 |
| lymphocyte act | | | |
| CD45RO CD4 | 31.2 | Coronery artery SMC | 4.5 |
| lymphocyte act | | TNF alpha + IL-1beta | |
| CD8 lymphocyte act | 25.5 | Astrocytes rest | 11.0 |
| Secondary CD8 | 27.4 | Astrocytes TNF alpha + | 5.0 |
| lymphocyte rest | | IL-1beta | |
| Secondary CD8 | 17.8 | KU-812 (Basophil) rest | 25.5 |
| lymphocyte act | | | |
| CD4 lymphocyte | 28.9 | KU-812 (Basophil) | 61.1 |
| none | | PMA/ionomycin | |
| 2ry | 32.5 | CCD1106 (Keratinocytes) | 14.4 |
| Th1/Th2/Tr1_anti-CD95 CH11 | | none | |
| LAK cells rest | 29.1 | CCD1106 (Keratinocytes) | 0.5 |
| | | TNF alpha + IL-1beta | |
| LAK cells IL-2 | 42.3 | Liver cirrhosis | 4.5 |
| LAK cells IL-2 + IL- | 31.4 | Lupus kidney | 6.7 |
| 12 | | | |
| LAK cells IL-2 + IFN | 64.6 | NCI-H292 none | 46.7 |
| gamma | | | |
| LAK cells IL-2 + IL- | 81.8 | NCI-H292 IL-4 | 68.3 |
| 18 | | | |
| LAK cells | 20.3 | NCI-H292 IL-9 | 46.0 |
| PMA/ionomycin | | | |
| NK Cells IL-2 rest | 28.9 | NCI-H292 IL-13 | 24.5 |
| Two Way MLR 3 | 46.7 | NCI-H292 IFN gamma | 35.8 |
| day | | | |
| Two Way MLR 5 | 12.0 | HPAEC none | 10.2 |
| day | | | |
| Two Way MLR 7 | 7.9 | HPAEC TNF alpha + IL- | 9.4 |
| day | | 1beta | |
| PBMC rest | 13.5 | Lung fibroblast none | 8.2 |
| PBMC PWM | 81.8 | Lung fibroblast TNF | 10.4 |
| | | alpha + IL-1beta | |
| PBMC PHA-L | 29.7 | Lung fibroblast IL-4 | 17.6 |
| Ramos (B cell) none | 37.4 | Lung fibroblast IL-9 | 17.8 |
| Ramos (B cell) | 100.0 | Lung fibroblast IL-13 | 11.8 |
| ionomycin | | | |
| B lymphocytes | 66.4 | Lung fibroblast IFN | 14.3 |
| PWM | | gamma | |
| B lymphocytes | 67.4 | Dermal fibroblast | 20.9 |
| CD40L and IL-4 | | CCD1070 rest | |
| EOL-1 dbcAMP | 20.9 | Dermal fibroblast | 73.7 |
| | | CCD1070 TNF alpha | |
| EOL-1 dbcAMP | 22.4 | Dermal fibroblast | 14.0 |
| PMA/ionomycin | | CCD1070 IL-1beta | |
| Dendritic cells none | 11.6 | Dermal fibroblast IFN | 9.0 |
| | | gamma | |
| Dendritic cells LPS | 11.3 | Dermal fibroblast IL-4 | 17.0 |
| Dendritic cells anti- | 19.8 | IBD Colitis 2 | 2.8 |
| CD40 | | | |
| Monocytes rest | 19.1 | IBD Crohn's | 5.2 |
| Monocytes LPS | 18.9 | Colon | 20.9 |
| Macrophages rest | 11.1 | Lung | 8.3 |
| Macrophages LPS | 11.7 | Thymus | 29.3 |
| HUVEC none | 10.0 | Kidney | 51.1 |
| HUVEC starved | 23.3 | | |

CNS_neurodegeneration_v1.0 Summary: Ag3142 This panel confirms the expression of this gene at moderate to low levels in the brain in an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag3142 The CG57226-01 gene is expressed at low to moderate levels in the majority of samples on this panel, with highest expression in ascites derived ovarian cancer cell line OVCAR-5 (CT=30.4). It is also expressed in melanoma, prostate, breast, lung, renal, gastric, colon, neuroblastoma, pancreatic and liver adenocarcinoma cell lines. Hence, this gene is likely to play a role in cell survival and proliferation.

Among tissues with metabolic or endocrine function, this gene is expressed in pituitary gland, liver, adipose, and pancreas. Therefore, therapeutic modulation of the activity of this gene may prove useful in the treatment of endocrine/metabolically related diseases, such as obesity and diabetes.

This gene is expressed at low levels throughout the CNS, including in amygdala, hippocampus, substantia nigra, thalamus, cerebellum, and cerebral cortex. Therefore, this gene may play a role in central nervous system disorders such as Alzheimer's disease, Parkinson's disease, epilepsy, multiple sclerosis, schizophrenia and depression.

Panel 4D Summary: Ag3142 The CG57226-01 gene is expressed at moderate levels in a wide range of cell types of significance in the immune response in health and disease. These cells include members of the T-cell, B-cell, endothelial cell, macrophage/monocyte/dendritic cell, basophil, eosinophil and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues.

This pattern is in agreement with the expression profile in Panel 1.3D and also suggests a role for the gene product in cell survival and proliferation.

Therefore, therapeutic modulation of the activity of this gene or its protein product may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and ostcoartliritis.

NOV43

Expression of NOV43/CG57538-01 was assessed using the primer-probe set Ag3277, described in Table AKA. Results of the RTQ-PCR runs are shown in Table AKB.

TABLE AKA
Probe Name Ag3277

| Primers | Sequences | Length | Start Position | SEQ ID NO |
|---|---|---|---|---|
| Forward | 5′-caaccctggtcaaataattcaa-3′ | 22 | 2424 | 386 |
| Probe | TET-5′-aaaataaagccgcaagaccgtattct-3′-TAMRA | 26 | 2456 | 387 |
| Reverse | 5′-ttttcactccatgagcatgaat-3′ | 22 | 2482 | 388 |

TABLE AKB
Panel 4D

| | Rel. Exp. (%) Ag3277, Run | | Rel. Exp. (%) Ag3277, Run |
|---|---|---|---|
| Tissue Name | 164635110 | Tissue Name | 164635110 |
| Secondary Th1 act | 0.0 | HUVEC IL-1beta | 0.0 |
| Secondary Th2 act | 0.0 | HUVEC IFN gamma | 2.6 |
| Secondary Tr1 act | 0.0 | HUVEC TNF alpha + IFN gamma | 8.7 |
| Secondary Th1 rest | 2.8 | HUVEC TNF alpha + IL4 | 2.3 |
| Secondary Th2 rest | 0.0 | HUVEC IL-11 | 0.0 |
| Secondary Tr1 rest | 0.0 | Lung Microvascular EC none | 4.0 |
| Primary Th1 act | 0.0 | Lung Microvascular EC TNF alpha + | 4.5 |
| | | IL-1beta | |
| Primary Th2 act | 0.0 | Microvascular Dermal EC none | 6.4 |
| Primary Tr1 act | 0.0 | Microsvasular Dermal EC | 0.0 |
| | | TNF alpha + IL-1beta | |
| Primary Th1 rest | 0.0 | Bronchial epithelium TNF alpha + | 0.0 |
| | | IL-1beta | |
| Primary Th2 rest | 6.7 | Small airway epithelium none | 0.0 |
| Primary Tr1 rest | 0.0 | Small airway epithelium TNF alpha + | 7.6 |
| | | IL1-beta | |
| CD45RA CD4 lymphocyte | 0.0 | Coronery artery SMC rest | 0.0 |
| act | | | |
| CD45RO CD4 lymphocyte | 5.1 | Coronery artery SMC TNF alpha + | 0.0 |
| act | | IL-1beta | |
| CD8 lymphocyte act | 0.0 | Astrocytes rest | 0.0 |
| Secondary CD8 lymphocyte | 0.0 | Astrocytes TNF alpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 44.8 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 6.1 |
| LAK cells IL-2 + IL-12 | 0.0 | Lupus kidney | 4.1 |
| LAK cells IL-2 + IFN gamma | 0.0 | NCI-H292 none | 14.5 |
| LAK cells IL-2 + IL-18 | 0.0 | NCI-H292 IL-4 | 100.0 |
| LAK cells PMA/ionomycin | 0.0 | NCI-H292 IL-9 | 27.4 |
| NK Cells IL-2 rest | 17.4 | NCI-H292 IL-13 | 37.4 |
| Two Way MLR 3 day | 0.0 | NCI-H292 IFN gamma | 8.2 |
| Two Way MLR 5 day | 4.7 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 0.0 | Lung fibroblast TNF alpha + IL- | 0.0 |
| | | 1beta | |
| PBMC PHA-L | 3.9 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 rest | 0.0 |
| IL-4 | | | |
| EOL-1 dbcAMP | 0.0 | Dermal fibroblast CCD1070 TNF | 9.5 |
| | | alpha | |
| EOL-1 dbcAMP | 12.2 | Dermal fibroblast CCD1070 IL- | 0.0 |
| PMA/ionomycin | | 1beta | |
| Dendritic cells none | 0.0 | Dermal fibroblast IFN gamma | 0.0 |
| Dendritic cells LPS | 2.3 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 5.2 | Colon | 0.0 |
| Macrophages rest | 0.0 | Lung | 3.2 |
| Macrophages LPS | 0.0 | Thymus | 0.0 |
| HUVEC none | 4.2 | Kidney | 15.1 |
| HUVEC starved | 0.0 | | |

CNS_neurodegeneration_v1.0 Summary: Ag3277 Expression of this gene is low/undetectable (CTs>35) across all of the samples on this panel (data not shown).

General_screening_panel_v1.4 Summary: Ag3277 Results from one experiment with the CG57538-01 gene are not included. The amp plot indicates that there were experimental difficulties with this run (data not shown).

Panel 4D Summary: Ag3277 Highest expression of the CG57538-01 gene, a Ceruloplasmin homolog, is in samples derived from the pulmonary mucoepidermoid cell line NCI-H292 stimulated with IL-4 (CTs=34.4). Thus, this expression profile indicates that this gene product may play a key role as a mediator of inflammation, especially in late-phase allergic reactions, and as a mediator of local cellular movement or trafficking into the inflamed area by cytokines and chemokines. Airway epithelial cells are believed to be the major source of Ceruloplasmin in the lung fluid and may play critical role in host defense against oxidative damage and infection in the lung (ref. 1).

References:

1. Yang F, Friedrichs W E, deGraffenried L, Herbert D C, Weaker F J, Bowman B H, Coalson J J. Cellular expression of ceruloplasmin in baboon and mouse lung during development and inflammation. Am J Respir Cell Mol Biol 1996 February; 14(2):161-9

Abstract—Ceruloplasmin (CP) is an important extracellular antioxidant and free radical scavenger. Although CP is expressed mainly in the liver, recent studies have identified the lung as another major site of CP synthesis. The sites and cell types that are responsible for CP expression in baboon and mouse lung are described. CP mRNA is detected in primordial bronchial epithelium in baboon fetuses by 60 days of gestation. At 140 days of gestation and thereafter, CP mRNA is found in airway epithelium and in the ductal cells of the submucosal glands. In developing and mature mice, CP mRNA is present in epithelial cells throughout the airway. In endotoxin-treated mice, the amount of CP mRNA increases several-fold in large airways but increases only moderately in small airways. This suggests that the high concentration of CP in the mucus lining of the upper airway, which serves to filter harmful substances, is particularly important during stressful conditions. Endotoxin treatment in mice also results in the induction of high levels of CP mRNA in a subset of alveolar wall cells. The data suggest that the airway epithelial cells are the major source of CP in the lung fluid and support ceruloplasmin's critical role in host defense against oxidative damage and infection in the lung.

NOV44

Expression of NOV44A/CG57623-01 and NOV44B/CG57623-02 was assessed using the primer-probe set Ag3296, described in Table AKA. Results of the RTQ-PCR runs are shown in Tables AKB, AKC and AKD. Please note that CG57623-02 represents a full-length physical clone of the CG57623-01 gene, validating the prediction of the gene sequence.

TABLE ALA
Probe Name Ag3296

| | | | Start | |
|---|---|---|---|---|
| Primers | Sequences | Length | Position | SEQ ID NO |
| Forward | 5′-agaccatctttcccaatctgat-3′ | 22 | 737 | 389 |
| Probe | TET-5′-caattccatggccaaggactcctg-3′-TAMRA | 24 | 765 | 390 |
| Reverse | 5′-gttccaagtcacctccagtctt-3′ | 22 | 791 | 391 |

TABLE ALB
CNS_neurodegeneration_v1.0

| | Rel. Exp. (%) Ag3296, Run | | Rel. Exp. (%) Ag3296, Run |
|---|---|---|---|
| Tissue Name | 210063000 | Tissue Name | 210063000 |
| AD 1 Hippo | 19.5 | Control (Path) 3 Temporal | 9.8 |
| | | Ctx | |
| AD 2 Hippo | 3.0 | Control (Path) 4 Temporal | 4.3 |
| | | Ctx | |
| AD 3 Hippo | 6.5 | AD 1 Occipital Ctx | 10.9 |
| AD 4 Hippo | 39.5 | AD 2 Occipital Ctx | 0.0 |
| | | (Missing) | |
| AD 5 Hippo | 26.6 | AD 3 Occipital Ctx | 10.4 |
| AD 6 Hippo | 84.7 | AD 4 Occipital Ctx | 6.5 |
| Control 2 Hippo | 10.8 | AD 5 Occipital Ctx | 5.1 |
| Control 4 Hippo | 33.7 | AD 6 Occipital Ctx | 0.0 |
| Control (Path) 3 Hippo | 16.4 | Control 1 Occipital Ctx | 12.6 |
| AD 1 Temporal Ctx | 18.3 | Control 2 Occipital Ctx | 7.4 |
| AD 2 Temporal Ctx | 4.7 | Control 3 Occipital Ctx | 0.0 |
| AD 3 Temporal Ctx | 4.3 | Control 4 Occipital Ctx | 4.2 |
| AD 4 Temporal Ctx | 15.8 | Control (Path) 1 Occipital | 12.5 |
| | | Ctx | |
| AD 5 Inf Temporal Ctx | 100.0 | Control (Path) 2 Occipital | 0.0 |
| | | Ctx | |
| AD 5 Sup Temporal Ctx | 83.5 | Control (Path) 3 Occipital | 2.3 |
| | | Ctx | |
| AD 6 Inf Temporal Ctx | 39.0 | Control (Path) 4 Occipital | 0.0 |
| | | Ctx | |
| AD 6 Sup Temporal Ctx | 18.0 | Control 1 Parietal Ctx | 12.6 |
| Control 1 Temporal Ctx | 0.0 | Control 2 Parietal Ctx | 13.2 |
| Control 2 Temporal Ctx | 13.5 | Control 3 Parietal Ctx | 9.7 |
| Control 3 Temporal Ctx | 4.7 | Control (Path) 1 Parietal | 24.5 |
| | | Ctx | |
| Control 3 Temporal Ctx | 15.8 | Control (Path) 2 Parietal | 0.0 |
| | | Ctx | |
| Control (Path) 1 Temporal | 16.3 | Control (Path) 3 Parietal | 0.0 |
| Ctx | | Ctx | |
| Control (Path) 2 Temporal | 10.3 | Control (Path) 4 Parietal | 3.3 |
| Ctx | | Ctx | |

TABLE ALC
General_screening_panel_v1.4

| | Rel. Exp. (%) Ag3296, Run | | Rel. Exp. (%) Ag3296, Run |
|---|---|---|---|
| Tissue Name | 215669667 | Tissue Name | 215669667 |
| Adipose | 6.3 | Renal ca. TK-10 | 0.0 |
| Melanoma* Hs688(A).T | 0.0 | Bladder | 25.7 |
| Melanoma* Hs688(B).T | 0.0 | Gastric ca. (liver met.) NCI- | 0.0 |
| | | N87 | |
| Melanoma* M14 | 0.0 | Gastric ca. KATO III | 0.0 |
| Melanoma* LOXIMVI | 0.0 | Colon ca SW-948 | 0.0 |
| Melanoma* SK-MEL-5 | 1.2 | Colon ca. SW480 | 0.0 |
| Squamous cell carcinoma | 0.0 | Colon ca.* (SW480 met) | 0.0 |
| SCC-4 | | SW620 | |
| Testis Pool | 47.6 | Colon ca. HT29 | 0.0 |
| Prostate ca.* (bone met) | 0.0 | Colon ca. HCT-116 | 0.0 |
| PC-3 | | | |
| Prostate Pool | 4.1 | Colon ca. CaCo-2 | 0.0 |
| Placenta | 1.1 | Colon cancer tissue | 12.9 |
| Uterus Pool | 3.0 | Colon ca. SW1116 | 0.0 |
| Ovarian ca. OVCAR-3 | 0.0 | Colon ca. Colo-205 | 0.0 |
| Ovarian ca. SK-OV-3 | 1.8 | Colon ca. SW-48 | 0.0 |
| Ovarian ca. OVCAR-4 | 1.0 | Colon Pool | 19.5 |
| Ovarian ca. OVCAR-5 | 6.8 | Small Intestine Pool | 10.4 |
| Ovarian ca. IGROV-1 | 0.0 | Stomach Pool | 6.3 |
| Ovarian ca. OVCAR-8 | 0.0 | Bone Marrow Pool | 4.3 |
| Ovary | 18.9 | Fetal Heart | 5.7 |
| Breast ca. MCF-7 | 0.0 | Heart Pool | 5.3 |
| Breast ca. MDA-MB-231 | 0.0 | Lymph Node Pool | 14.6 |
| Breast ca. BT 549 | 0.0 | Fetal Skeletal Muscle | 12.2 |
| Breast ca. T47D | 5.6 | Skeletal Muscle Pool | 3.3 |
| Breast ca. MDA-N | 0.0 | Spleen Pool | 87.1 |
| Breast Pool | 25.7 | Thymus Pool | 25.3 |
| Trachea | 26.2 | CNS cancer (glio/astro) U87- | 0.9 |
| | | MG | |
| Lung | 2.2 | CNS canccr (glio/astro) U- | 7.5 |
| | | 118-MG | |
| Fetal Lung | 100.0 | CNS cancer (neuro; met) SK- | 0.0 |
| | | N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 11.3 |
| Lung ca. NCI-H526 | 0.0 | Brain (cerebellum) | 1.3 |
| Lung ca. NCI-H23 | 22.4 | Brain (fetal) | 1.2 |
| Lung ca. NCI-H460 | 5.7 | Brain (Hippocampus) Pool | 10.7 |
| Lung ca. HOP-62 | 0.0 | Cerebral Cortex Pool | 2.4 |
| Lung ca. NCI-H522 | 0.0 | Brain (Substantia nigra) Pool | 4.4 |
| Liver | 2.4 | Brain (Thalamus) Pool | 7.1 |
| Fetal Liver | 8.4 | Brain (whole) | 3.4 |
| Liver ca. HepG2 | 0.0 | Spinal Cord Pool | 11.0 |
| Kidney Pool | 13.8 | Adrenal Gland | 8.4 |
| Fetal Kidney | 17.8 | Pituitary gland Pool | 3.4 |
| Renal ca. 786-0 | 0.0 | Salivary Gland | 3.3 |
| Renal ca. A498 | 0.0 | Thyroid (female) | 1.5 |
| Renal ca. ACHN | 0.0 | Pancreatic ca. CAPAN2 | 0.0 |
| Renal ca. UO-31 | 0.0 | Pancreas Pool | 16.8 |

TABLE ALD
Panel 4D

| | Rel. Exp. (%) Ag3296, Run | | Rel. Exp. (%) Ag3296, Run |
|---|---|---|---|
| Tissue Name | 164633942 | Tissue Name | 164633942 |
| Secondary Th1 act | 0.2 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.2 | Lung Microvascular EC TNF alpha + | 0.0 |
| | | IL-1beta | |
| Primary Th2 act | 0.6 | Microvascular Dermal EC none | 0.0 |
| Primary Tr1 act | 0.0 | Microsvasular Dermal EC | 0.0 |
| | | TNF alpha + IL-1beta | |
| Primiary Th1 rest | 0.6 | Bronchial epithelium TNF alpha + | 0.0 |
| | | IL-1beta | |
| Primary Th2 rest | 0.5 | Small airway epithelium none | 0.0 |
| Primary Tr1 rest | 0.5 | Small airway epithelium TNF alpha + | 0.3 |
| | | IL-1beta | |
| CD45RA CD4 lymphocyte | 3.1 | Coronery artery SMC rest | 0.0 |
| act | | | |
| CD45RO CD4 lymphocyte | 2.0 | Coronery artery SMC TNF alpha + | 0.0 |
| act | | IL-1beta | |
| CD8 lymphocyte act | 0.6 | Astrocytes rest | 0.0 |
| Secondary CD8 lymphocyte | 0.0 | Astrocytes TNF alpha + IL-1beta | 0.0 |
| rest | | | |
| Secondary CD8 lymphocyte | 0.0 | KU-812 (Basophil) rest | 0.0 |
| act | | | |
| CD4 lymphocyte none | 1.9 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 18.7 | CCD1106 (Keratinocytes) | 0.0 |
| | | TNF alpha + IL-1beta | |
| LAK cells IL-2 | 0.9 | Liver cirrhosis | 1.4 |
| LAK cells IL-2 + IL-12 | 5.5 | Lupus kidney | 0.0 |
| LAK cells IL-2 + IFN gamma | 4.6 | NCI-H292 none | 0.0 |
| LAK cells IL-2 + IL-18 | 4.8 | NCI-H292 IL-4 | 0.0 |
| LAK cells PMA/ionomycin | 21.2 | NCI-H292 IL-9 | 0.0 |
| NK Cells IL-2 rest | 2.7 | NCI-H292 IL-13 | 0.0 |
| Two Way MLR 3 day | 12.0 | NCI-H292 IFN gamma | 0.0 |
| Two Way MLR 5 day | 3.7 | HPAEC none | 0.0 |
| Two Way MLR 7 day | 0.8 | HPAEC TNF alpha + IL-1beta | 0.0 |
| PBMC rest | 8.1 | Lung fibroblast none | 0.0 |
| PBMC PWM | 12.6 | Lung fibroblast TNF alpha + IL- | 0.0 |
| | | 1beta | |
| PBMC PHA-L | 14.9 | Lung fibroblast IL-4 | 0.3 |
| Ramos (B cell) none | 21.2 | Lung fibroblast IL-9 | 0.2 |
| Ramos (B cell) ionomycin | 83.5 | Lung fibroblast IL-13 | 0.3 |
| B lymphocytes PWM | 39.5 | Lung fibroblast IFN gamma | 0.3 |
| B lymphocytes CD40L and | 100.0 | Dermal fibroblast CCD1070 rest | 0.0 |
| IL-4 | | | |
| EOL-1 dbcAMP | 18.4 | Dermal fibroblast CCD1070 TNF | 0.4 |
| | | alpha | |
| EOL-1 dbcAMP | 55.5 | Dermal fibroblast CCD1070 IL- | 0.0 |
| PMA/ionomycin | | 1beta | |
| Dendritic cells none | 21.0 | Dermal fibroblast IFN gamma | 0.0 |
| Dendritic cells LPS | 10.3 | Dermal fibroblast IL-4 | 0.0 |
| Dendritic cells anti-CD40 | 35.8 | IBD Colitis 2 | 1.6 |
| Monocytes rest | 25.9 | IBD Crohn's | 0.0 |
| Monocytes LPS | 11.3 | Colon | 4.6 |
| Macrophages rest | 27.5 | Lung | 4.5 |
| Macrophages LPS | 13.7 | Thymus | 2.2 |
| HUVEC none | 0.0 | Kidney | 15.3 |
| HUVEC starved | 0.0 | | |

CNS_neurodegeneration_v1.0 Summary: Ag3296 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3296 Expression of this gene is primarily associated with normal tissues rather than cancer cell lines. Expression of the CG57623-01 gene is highest in the fetal lung (CT=31.1). Interestingly, this gene is expressed at higher levels in fetal lung (CT=31.1) when compared to adult lung (CT=36.6), suggesting that expression of this gene can be used to distinguish fetal from adult lung.

This gene is also expressed at low levels in several regions of the brain, including amygdala, hippocampus, thalamus and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at low levels in adrenal gland, pancreas, fetal skeletal muscle, and fetal liver. Therefore, therapeutic modulation of the activity of this gene may prove useful in the treatment of endocrine/metabolically related diseases, such as obesity and diabetes.

Panel 4D Summary: Ag3296 The CG57623-01 gene is expressed at highest levels in activated B lymphocytes, represented by ionomycin-activated Ramos and CD40L and IL-4 or pokeweed mitogen-activated B lymphocytes. Therefore, small molecule drugs or antibodies that antagonize the function of this gene product may be useful as therapeutic drugs to reduce or eliminate the symptoms in patients with autoimmune and inflammatory diseases in which B cells play a part in the initiation or progression of the disease process, such as lupus erythematosus, Crohn's disease, ulcerative colitis, multiple sclerosis, chronic obstructive pulmonary disease, asthma, emphysema, rheumatoid arthritis, or psoriasis.

NOV45

Expression of NOV45A/CG57656-01 and NOV45B/CG57656-02 was assessed using the primer-probe sets Ag3298 and Ag4482, described in Tables AMA and AMB. Results of the RTQ-PCR runs are shown in Tables AMC, AMD, AME and AMF. Please note that primer-probe set Ag4482 is specific for CG57656-02.

TABLE AMA
Probe Name Ag3298

| Primers | Sequences | Length | Start Position | SEQ ID NO |
|---|---|---|---|---|
| Forward | 5′-CAGACCTATGCAACCTCTGGTA-3′ | 22 | 3390 | 392 |
| Probe | TET-5′-agctgcgacatacaagccaaggcat-3′-TAMRA | 25 | 3422 | 393 |
| Reverse | 5′-ggtaaggcagcacaggtatg-3′ | 20 | 3450 | 394 |

TABLE AMB
Probe Name Ag4482

| Primers | Sequences | Length | Start Position | SEQ ID NO |
|---|---|---|---|---|
| Forward | 5′-gaggtggtcactgtgatcactt-3′ | 22 | 1804 | 395 |
| Probe | TET-5′-ttacaactccagaacccctggtgctg-3′-TAMRA | 26 | 1838 | 396 |
| Reverse | 5′-ctgagtccgattggctatgag-3′ | 21 | 1882 | 397 |

TABLE AMC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3298, Run | Ag4482, Run | | Ag3298, Run | Ag4482, Run |
| Tissue Name | 210063510 | 224535697 | Tissue Name | 210063510 | 224535697 |
| AD 1 Hippo | 6.0 | 6.9 | Control (Path) 3 | 20.3 | 3.9 |
| | | | Temporal Ctx | | |
| AD 2 Hippo | 25.5 | 24.7 | Control (Path) 4 | 54.3 | 43.2 |
| | | | Temporal Ctx | | |
| AD 3 Hippo | 9.5 | 6.2 | AD 1 Occipital | 48.0 | 12.9 |
| | | | Ctx | | |
| AD 4 Hippo | 16.0 | 8.4 | AD 2 Occipital | 0.0 | 0.0 |
| | | | Ctx (Missing) | | |
| AD 5 Hippo | 100.0 | 100.0 | AD 3 Occipital | 17.6 | 6.6 |
| | | | Ctx | | |
| AD 6 Hippo | 34.6 | 27.7 | AD 4 Occipital | 38.7 | 20.2 |
| | | | Ctx | | |
| Control 2 Hippo | 19.9 | 12.3 | AD 5 Occipital | 30.1 | 42.6 |
| | | | Ctx | | |
| Control 4 Hippo | 23.2 | 10.0 | AD 6 Occipital | 47.6 | 23.7 |
| | | | Ctx | | |
| Control (Path) 3 | 12.9 | 5.5 | Control 1 | 10.0 | 3.1 |
| Hippo | | | Occipital Ctx | | |
| AD 1 Temporal | 42.3 | 12.3 | Control 2 | 43.2 | 38.7 |
| Ctx | | | Occipital Ctx | | |
| AD 2 Temporal | 35.4 | 34.6 | Control 3 | 66.4 | 22.2 |
| Ctx | | | Occipital Ctx | | |
| AD 3 Temporal | 24.3 | 9.1 | Control 4 | 20.7 | 4.8 |
| Ctx | | | Occipital Ctx | | |
| AD 4 Temporal | 61.6 | 24.3 | Control (Path) 1 | 76.3 | 96.6 |
| Ctx | | | Occipital Ctx | | |
| AD 5 Inf | 79.0 | 59.9 | Control (Path) 2 | 38.7 | 17.1 |
| Temporal Ctx | | | Occipital Ctx | | |
| AD 5 Sup | 45.1 | 39.0 | Control (Path) 3 | 4.9 | 1.4 |
| Temporal Ctx | | | Occipital Ctx | | |
| AD 6 Inf | 65.1 | 42.6 | Control (Path) 4 | 67.8 | 32.1 |
| Temporal Ctx | | | Occipital Ctx | | |
| AD 6 Sup | 64.6 | 57.4 | Control 1 | 27.9 | 7.8 |
| Temporal Ctx | | | Parietal Ctx | | |
| Control 1 | 25.9 | 6.7 | Control 2 | 70.2 | 49.3 |
| Temporal Ctx | | | Parietal Ctx | | |
| Control 2 | 30.1 | 22.1 | Control 3 | 34.6 | 18.4 |
| Temporal Ctx | | | Parietal Ctx | | |
| Control 3 | 56.6 | 19.5 | Control (Path) 1 | 61.1 | 68.8 |
| Temporal Ctx | | | Parietal Ctx | | |
| Control 3 | 31.6 | 13.6 | Control (Path) 2 | 46.7 | 33.0 |
| Temporal Ctx | | | Parietal Ctx | | |
| Control (Path) 1 | 71.2 | 76.8 | Control (Path) 3 | 7.6 | 3.4 |
| Temporal Ctx | | | Parietal Ctx | | |
| Control (Path) 2 | 76.3 | 50.0 | Control (Path) 4 | 78.5 | 54.7 |
| Temporal Ctx | | | Parietal Ctx | | |

TABLE AMD
General_screening_panel_v1.4

| | Rel. Exp. (%) Ag3298, | Rel. Exp. (%) Ag3298, | Rel. Exp. (%) Ag3298, | Rel. Exp. (%) Ag4482, |
|---|---|---|---|---|
| Tissue Name | Run 215601953 | Run 216607677 | Run 222691295 | Run 222666310 |
| Adipose | 0.2 | 0.9 | 1.9 | 0.7 |
| Melanoma* Hs688(A).T | 0.0 | 0.0 | 0.0 | 0.0 |
| Melanoma* Hs688(B).T | 0.1 | 0.2 | 0.0 | 0.1 |
| Melanoma* M14 | 0.0 | 0.0 | 0.0 | 0.0 |
| Melanoma* LOXIMVI | 2.1 | 2.5 | 3.3 | 1.7 |
| Melanoma* SK-MEL-5 | 2.4 | 2.2 | 1.5 | 3.7 |
| Squamous cell | 0.1 | 0.2 | 0.1 | 0.1 |
| carcinoma SCC-4 | | | | |
| Testis Pool | 5.5 | 7.3 | 7.9 | 5.6 |
| Prostate ca.* (bone met) | 0.9 | 1.4 | 0.5 | 1.1 |
| PC-3 | | | | |
| Prostate Pool | 5.5 | 4.5 | 4.5 | 2.4 |
| Placenta | 0.5 | 0.7 | 0.7 | 0.6 |
| Uterus Pool | 2.5 | 1.5 | 1.6 | 2.7 |
| Ovarian ca. OVCAR-3 | 0.0 | 0.1 | 0.0 | 0.0 |
| Ovarian ca. SK-OV-3 | 0.0 | 0.0 | 0.0 | 0.0 |
| Ovarian ca. OVCAR-4 | 0.1 | 0.0 | 0.0 | 0.1 |
| Ovarian ca. OVCAR-5 | 20.2 | 19.1 | 21.6 | 13.7 |
| Ovarian ca. IGROV-1 | 0.0 | 0.4 | 0.4 | 0.1 |
| Ovarian ca. OVCAR-8 | 0.0 | 0.0 | 0.0 | 0.0 |
| Ovary | 0.5 | 0.7 | 0.3 | 0.6 |
| Breast ca. MCF-7 | 3.0 | 2.1 | 2.1 | 1.6 |
| Breast ca. MDA-MB- | 0.0 | 0.0 | 0.0 | 0.0 |
| 231 | | | | |
| Breast ca. BT 549 | 0.6 | 0.2 | 0.5 | 0.2 |
| Breast ca. T47D | 45.4 | 52.5 | 43.5 | 17.0 |
| Breast ca. MDA-N | 0.0 | 0.0 | 0.0 | 0.0 |
| Breast Pool | 10.4 | 10.8 | 12.5 | 9.0 |
| Trachea | 1.4 | 4.9 | 4.6 | 2.5 |
| Lung | 0.8 | 1.0 | 0.7 | 0.5 |
| Fetal Lung | 6.5 | 7.0 | 5.7 | 5.2 |
| Lung ca. NCI-N417 | 16.3 | 14.8 | 13.6 | 4.0 |
| Lung ca. LX-1 | 12.9 | 15.7 | 11.4 | 13.1 |
| Lung ca. NCI-H146 | 4.3 | 5.6 | 5.2 | 2.2 |
| Lung ca. SHP-77 | 7.4 | 5.2 | 5.0 | 7.9 |
| Lung ca. A549 | 2.2 | 1.1 | 1.1 | 1.1 |
| Lung ca. NCI-H526 | 25.5 | 21.6 | 18.7 | 6.8 |
| Lung ca. NCI-H23 | 9.2 | 4.9 | 4.1 | 3.8 |
| Lung ca. NCI-H460 | 1.4 | 0.4 | 1.0 | 0.5 |
| Lung ca. HOP-62 | 0.2 | 0.0 | 0.2 | 0.1 |
| Lung ca. NCI-H522 | 3.5 | 2.2 | 4.2 | 3.2 |
| Liver | 0.0 | 0.0 | 0.0 | 0.0 |
| Fetal Liver | 0.1 | 0.4 | 0.1 | 0.1 |
| Liver ca. HepG2 | 0.0 | 0.0 | 0.0 | 0.0 |
| Kidney Pool | 51.8 | 62.0 | 59.0 | 27.0 |
| Fetal Kidney | 3.1 | 3.9 | 2.6 | 1.4 |
| Renal ca. 786-0 | 0.0 | 0.0 | 0.0 | 0.0 |
| Renal ca. A498 | 0.1 | 0.1 | 0.2 | 0.0 |
| Renal ca. ACHN | 0.0 | 0.1 | 0.2 | 0.2 |
| Renal ca. UO-31 | 2.6 | 1.2 | 2.0 | 0.9 |
| Renal ca. TK-10 | 1.5 | 1.9 | 1.7 | 2.3 |
| Bladder | 2.2 | 1.5 | 1.7 | 1.2 |
| Gastric ca. (liver met.) | 100.0 | 93.3 | 94.0 | 100.0 |
| NCI-N87 | | | | |
| Gastric ca. KATO III | 97.3 | 85.3 | 100.0 | 62.4 |
| Colon ca. SW-948 | 8.1 | 6.9 | 7.7 | 2.0 |
| Colon ca. SW480 | 32.5 | 37.6 | 16.4 | 23.7 |
| Colon ca.* (SW480 | 4.3 | 6.6 | 3.9 | 4.6 |
| met) SW620 | | | | |
| Colon ca. HT29 | 2.5 | 3.3 | 3.8 | 2.8 |
| Colon ca. HCT-116 | 9.3 | 11.7 | 12.7 | 8.7 |
| Colon ca. CaCo-2 | 2.2 | 1.9 | 2.9 | 2.1 |
| Colon cancer tissue | 0.4 | 0.9 | 0.8 | 0.3 |
| Colon ca. SW1116 | 13.7 | 15.2 | 11.9 | 4.2 |
| Colon ca. Colo-205 | 15.4 | 8.9 | 12.4 | 2.8 |
| Colon ca. SW-48 | 0.0 | 0.0 | 0.0 | 0.1 |
| Colon Pool | 13.4 | 17.9 | 16.8 | 11.0 |
| Small Intestine Pool | 19.3 | 19.2 | 19.8 | 12.0 |
| Stomach Pool | 6.6 | 7.2 | 7.9 | 4.5 |
| Bone Marrow Pool | 2.4 | 4.3 | 6.6 | 5.5 |
| Fetal Heart | 9.0 | 12.3 | 16.5 | 9.4 |
| Heart Pool | 6.2 | 8.6 | 8.0 | 5.3 |
| Lymph Node Pool | 22.1 | 18.2 | 25.5 | 15.8 |
| Fetal Skeletal Muscle | 2.4 | 2.6 | 1.9 | 1.2 |
| Skeletal Muscle Pool | 1.6 | 2.4 | 1.1 | 1.2 |
| Spleen Pool | 1.7 | 3.0 | 2.4 | 2.0 |
| Thymus Pool | 4.1 | 5.0 | 8.0 | 2.4 |
| CNS cancer (glio/astro) | 5.1 | 4.7 | 4.6 | 2.8 |
| U87-MG | | | | |
| CNS cancer (glio/astro) | 78.5 | 100.0 | 80.7 | 69.7 |
| U-118-MG | | | | |
| CNS cancer (neuro; met) | 5.1 | 5.3 | 6.2 | 4.9 |
| SK-N-AS | | | | |
| CNS cancer (astro) SF- | 0.0 | 0.0 | 0.0 | 0.0 |
| 539 | | | | |
| CNS cancer (astro) | 7.1 | 9.5 | 10.4 | 5.6 |
| SNB-75 | | | | |
| CNS cancer (glio) SNB- | 0.2 | 0.0 | 0.2 | 0.1 |
| 19 | | | | |
| CNS cancer (glio) SF- | 5.6 | 4.7 | 4.2 | 4.1 |
| 295 | | | | |
| Brain (Amygdala) Pool | 5.9 | 7.3 | 5.8 | 2.4 |
| Brain (cerebellum) | 59.5 | 56.3 | 55.5 | 37.9 |
| Brain (fetal) | 10.7 | 12.0 | 15.1 | 8.7 |
| Brain (Hippocampus) | 5.6 | 5.1 | 5.8 | 3.4 |
| Pool | | | | |
| Cerebral Cortex Pool | 7.5 | 7.9 | 7.3 | 4.4 |
| Brain (Substantia nigra) | 9.9 | 8.8 | 9.5 | 3.8 |
| Pool | | | | |
| Brain (Thalamus) Pool | 11.3 | 8.8 | 9.6 | 5.8 |
| Brain (whole) | 11.6 | 12.8 | 17.1 | 8.5 |
| Spinal Cord Pool | 2.0 | 2.6 | 5.3 | 2.0 |
| Adrenal Gland | 1.6 | 2.7 | 2.0 | 1.4 |
| Pituitary gland Pool | 2.5 | 2.8 | 2.4 | 1.4 |
| Salivary Gland | 0.2 | 0.3 | 0.5 | 0.3 |
| Thyroid (female) | 3.5 | 5.0 | 4.8 | 2.3 |
| Pancreatic ca. CAPAN2 | 8.2 | 4.9 | 6.4 | 6.8 |
| Pancreas Pool | 12.5 | 12.8 | 9.1 | 7.9 |

TABLE AME
Panel 4.1D

| | Rel. Exp. (%) Ag4482, Run | | Rel. Exp. (%) Ag4482, Run |
|---|---|---|---|
| Tissue Name | 195476203 | Tissue Name | 195476203 |
| Secondary Th1 act | 18.7 | HUVEC IL-1beta | 2.1 |
| Secondary Th2 act | 24.0 | HUVEC IFN gamma | 2.0 |
| Secondary Tr1 act | 15.1 | HUVEC TNF alpha + IFN gamma | 1.5 |
| Secondary Th1 rest | 40.6 | HUVEC TNF alpha + IL4 | 0.0 |
| Secondary Th2 rest | 43.2 | HUVEC IL-11 | 0.0 |
| Secondary Tr1 rest | 90.8 | Lung Microvascular EC none | 0.0 |
| Primary Th1 act | 54.7 | Lung Microvascular EC TNF alpha + | 0.5 |
| | | IL-1beta | |
| Primary Th2 act | 92.0 | Microvascular Dermal EC none | 0.0 |
| Primary Tr1 act | 75.3 | Microsvasular Dermal EC | 0.0 |
| | | TNF alpha + IL-1beta | |
| Primary Th1 rest | 60.3 | Bronchial epithelium TNF alpha + | 0.0 |
| | | IL1beta | |
| Primary Th2 rest | 49.0 | Small airway epithelium none | 0.0 |
| Primary Tr1 rest | 84.7 | Small airway epithelium TNF alpha + | 0.0 |
| | | IL-1beta | |
| CD45RA CD4 lymphocyte | 26.2 | Coronery artery SMC rest | 0.3 |
| act | | | |
| CD45RO CD4 lymphocyte | 42.0 | Coronery artery SMC TNF alpha + | 0.5 |
| act | | IL-1beta | |
| CD8 lymphocyte act | 33.9 | Astrocytes rest | 16.4 |
| Secondary CD8 lymphocyte | 60.7 | Astrocytes TNF alpha + IL-1beta | 15.3 |
| rest | | | |
| Secondary CD8 lymphocyte | 13.9 | KU-812 (Basophil) rest | 52.9 |
| act | | | |
| CD4 lymphocyte none | 41.8 | KU-812 (Basophil) | 92.7 |
| | | PMA/ionomycin | |
| 2ry Th1/Th2/Tr1_anti-CD95 | 68.8 | CCD1106 (Keratinocytes) none | 0.0 |
| CH11 | | | |
| LAK cells rest | 40.3 | CCD1106 (Keratinocytes) | 0.0 |
| | | TNF alpha + IL-1beta | |
| LAK cells IL-2 | 26.1 | Liver cirrhosis | 1.7 |
| LAK cells IL-2 + IL-12 | 32.8 | NCI-H292 none | 0.0 |
| LAK cells IL-2 + IFN gamma | 34.4 | NCI-H292 IL-4 | 0.0 |
| LAK cells IL-2 + IL-18 | 50.3 | NCI-H292 IL-9 | 1.3 |
| LAK cells PMA/ionomycin | 100.0 | NCI-H292 IL-13 | 0.7 |
| NK Cells IL-2 rest | 29.7 | NCI-H292 IFN gamma | 0.0 |
| Two Way MLR 3 day | 28.7 | HPAEC none | 0.6 |
| Two Way MLR 5 day | 21.8 | HPAEC TNF alpha + IL-1beta | 0.7 |
| Two Way MLR 7 day | 20.7 | Lung fibroblast none | 0.0 |
| PBMC rest | 14.1 | Lung fibroblast TNF alpha + IL- | 0.6 |
| | | 1beta | |
| PBMC PWM | 32.8 | Lung fibroblast IL-4 | 1.3 |
| PBMC PHA-L | 88.3 | Lung fibroblast IL-9 | 1.0 |
| Ramos (B cell) none | 0.0 | Lung fibroblast IL-13 | 0.6 |
| Ramos (B cell) ionomycin | 0.0 | Lung fibroblast IFN gamma | 1.6 |
| B lymphocytes PWM | 23.8 | Dermal fibroblast CCD1070 rest | 2.7 |
| B lymphocytes CD40L and | 9.7 | Dermal fibroblast CCD1070 TNF | 32.3 |
| IL-4 | | alpha | |
| EOL-1 dbcAMP | 0.6 | Dermal fibroblast CCD1070 IL- | 1.4 |
| | | 1beta | |
| EOL-1 dbcAMP | 0.0 | Dermal fibroblast IFN gamma | 1.4 |
| PMA/ionomycin | | | |
| Dendritic cells none | 5.0 | Dermal fibroblast IL-4 | 1.2 |
| Dendritic cells LPS | 0.8 | Dermal Fibroblasts rest | 2.2 |
| Dendritic cells anti-CD40 | 0.0 | Neutrophils TNFa + LPS | 0.8 |
| Monocytes rest | 0.6 | Neutrophils rest | 0.6 |
| Monocytes LPS | 0.0 | Colon | 19.3 |
| Macrophages rest | 0.8 | Lung | 3.1 |
| Macrophages LPS | 0.0 | Thymus | 18.9 |
| HUVEC none | 0.0 | Kidney | 7.5 |
| HUVEC starved | 0.0 | | |

TABLE AMF
Panel 4D

| | Rel. Exp. (%) Ag3298, Run | | Rel. Exp. (%) Ag3298, Run |
|---|---|---|---|
| Tissue Name | 164682520 | Tissue Name | 164682520 |
| Secondary Th1 act | 18.9 | HUVEC IL-1beta | 0.0 |
| Secondary Th2 act | 20.4 | HUVEC IFN gamma | 0.9 |
| Secondary Tr1 act | 19.6 | HUVEC TNF alpha + IFN gamma | 2.5 |
| Secondary Th1 rest | 40.3 | HUVEC TNF alpha + IL4 | 1.1 |
| Secondary Th2 rest | 34.4 | HUVEC IL-11 | 0.0 |
| Secondary Tr1 rest | 100.0 | Lung Microvascular EC none | 0.0 |
| Primary Th1 act | 38.2 | Lung Microsvasular EC TNF alpha + | 0.0 |
| | | IL-1beta | |
| Primary Th2 act | 56.3 | Microvascular Dermal EC none | 0.0 |
| Primary Tr1 act | 49.0 | Microvascular Dermal EC | 0.0 |
| | | TNF alpha + IL-1beta | |
| Primary Th1 rest | 87.1 | Bronchial epithelium TNF alpha + | 0.0 |
| | | IL1beta | |
| Primary Th2 rest | 63.7 | Small airway epithelium none | 0.0 |
| Primary Tr1 rest | 58.6 | Small airway epithelium TNF alpha + | 0.1 |
| | | IL-1beta | |
| CD45RA CD4 lymphocyte | 27.2 | Coronery artery SMC rest | 0.0 |
| act | | | |
| CD45RO CD4 lymphocyte | 31.9 | Coronery artery SMC TNF alpha + | 0.0 |
| act | | IL-1beta | |
| CD8 lymphocyte act | 46.0 | Astrocytes rest | 58.2 |
| Secondary CD8 lymphocyte | 41.8 | Astrocytes TNF alpha + IL-1beta | 13.2 |
| rest | | | |
| Secondary CD8 lymphocyte | 10.4 | KU-812 (Basophil) rest | 24.8 |
| act | | | |
| CD4 lymphocyte none | 26.1 | KU-812 (Basophil) | 52.9 |
| | | PMA/ionomycin | |
| 2ry Th1/Th2/Tr1_anti-CD95 | 31.9 | CCD1106 (Keratinocytes) none | 0.0 |
| CH11 | | | |
| LAK cells rest | 24.3 | CCD1106 (Keratinocytes) | 0.0 |
| | | TNF alpha + IL-1beta | |
| LAK cells IL-2 | 11.7 | Liver cirrhosis | 10.6 |
| LAK cells IL-2 + IL-12 | 29.3 | Lupus kidney | 0.0 |
| LAK cells IL-2 + IFN gamma | 35.8 | NCI-H292 none | 0.0 |
| LAK cells IL-2 + IL-18 | 32.3 | NCI-H292 IL-4 | 0.0 |
| LAK cells PMA/ionomycin | 59.5 | NCI-H292 IL-9 | 0.0 |
| NK Cells IL-2 rest | 21.8 | NCI-H292 IL-13 | 0.0 |
| Two Way MLR 3 day | 22.4 | NCI-H292 IFN gamma | 0.0 |
| Two Way MLR 5 day | 17.2 | HPAEC none | 0.0 |
| Two Way MLR 7 day | 17.0 | HPAEC TNF alpha + IL-1beta | 0.0 |
| PBMC rest | 15.1 | Lung fibroblast none | 0.0 |
| PBMC PWM | 37.9 | Lung fibroblast TNF alpha + IL- | 0.0 |
| | | 1beta | |
| PBMC PHA-L | 51.4 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 12.4 | Lung fibroblast IFN gamma | 2.4 |
| B lymphocytes CD40L and | 10.2 | Dermal fibroblast CCD1070 rest | 0.5 |
| IL-4 | | | |
| EOL-1 dbcAMP | 0.0 | Dermal fibroblast CCD1070 TNF | 12.8 |
| | | alpha | |
| EOL-1 dbcAMP | 0.0 | Dermal fibroblast CCD1070 IL- | 0.0 |
| PMA/ionomycin | | 1beta | |
| Dendritic cells none | 1.9 | Dermal fibroblast IFN gamma | 0.0 |
| Dendritic cells LPS | 0.0 | Dermal fibroblast IL-4 | 1.0 |
| Dendritic cells anti-CD40 | 0.0 | IBD Colitis 2 | 3.0 |
| Monocytes rest | 0.7 | IBD Crohn's | 0.0 |
| Monocytes LPS | 0.4 | Colon | 54.7 |
| Macrophages rest | 0.9 | Lung | 13.8 |
| Macrophages LPS | 1.4 | Thymus | 7.8 |
| HUVEC none | 1.0 | Kidney | 14.8 |
| HUVEC starved | 0.0 | | |

CNS_neurodegeneration_v1.0 Summary: Ag3298/Ag4482 Results from two experiments using different probe/primer sets are in excellent agreement. This panel confirms the expression of this gene at moderate levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3298/Ag4482 Results from four experiments using two different probe/primer sets are in excellent agreement, with highest expression of the CG57656-02 gene in two samples derived from gastric cancer cell lines (CTs=27.5). Interestingly, expression of this gene is much higher in the gastric cancer cell lines than in normal stomach. Thus, expression of this gene could be used to differentiate between these samples and other samples on this panel and as a marker for gastric cancer. Furthermore, therapeutic modulation of the activity of this gene or its protein product, using small molecule drugs, antibodies or protein therapeutics, could be of benefit in the treatment of gastric cancer.

This gene is also expressed at moderate levels throughout the CNS, including in amygdala, hippocampus, substantia nigra, thalamus, cerebral cortex, and spinal cord, and at high levels in the cerebellum. Therefore, this gene may play a role in central nervous system disorders such as Alzheimer's disease, Parkinson's disease, epilepsy, multiple sclerosis, schizophrenia and depression.

The CG57656-02 gene encodes a protein with similarity to the Drosophila turtle protein, a member of the Ig superfamily (IgSF). In Drosophila, the turtle (tut1) protein is expressed in the CNS and in a small, defined subset of the peripheral nervous system (PNS). Alternative splicing of the primary transcript results in membrane-bound and secreted Tut1 isoforms that are essential for development and adult viability. In addition, tut1 function is required for establishing a nervous system capable of executing complex forms of coordinated movement such as tactile escape response, coordinated righting behavior in larval and adult stages, and flight in adulthood (ref. 1).

Among tissues with metabolic or endocrine function, this gene is expressed at moderate levels in pancreas and heart and at low levels in thyroid, pituitary gland, adrenal gland, and skeletal muscle. Therefore, therapeutic modulation of the activity of this gene may prove useful in the treatment of endocrine/metabolically related diseases, such as obesity and diabetes.

References:

1. Bodily K D, Morrison C M, Renden R B, Broadie K. A novel member of the Ig superfamily, turtle, is a CNS specific protein required for coordinated motor control. J Neurosci May 1, 2001;21 (9):3113-25

We describe here the cloning and functional characterization of a neural-specific novel member of the Ig superfamily, turtle (tut1), with a structure of five Ig C2-type domains, two fibronectin type III domains, and one transmembrane region. Alternative splicing of the tut1 gene produces at least four Tut1 isoforms, including two transmembrane proteins and two secreted proteins, with primary structures closely related to a human brain protein (KIAA1355), the Deleted in Colorectal Cancer/Neogenin/Frazzled receptor family, and the Roundabout/Dutt1 receptor family. An allelic series of tut1 gene mutations resulted in recessive lethality to semilethality, indicating that the gene is essential. In contrast to other family members, tut1 does not play a detectable role in axon pathfinding or nervous system morphogenesis. Likewise, basal synaptic transmission and locomotory movement are unaffected. However, tut1 mutations cause striking movement defects exhibited in specific types of highly coordinated behavior. Specifically, tut1 mutants display an abnormal response to tactile stimulation, the inability to regain an upright position from an inverted position (hence, “turtle”), and the inability to fly in adulthood. These phenotypes demonstrate that tut1 plays an essential role in establishing a nervous system capable of executing coordinated motor output in complex behaviors.

PMID: 11312296

Panel 4.1D Summary: Ag4482 The CG57656-02 gene is expressed at highest levels in stimulated lymphokine-activated killer cells (LAK). LAK cells are involved in tumor immunology and cell clearance of virally and bacterial infected cells as well as tumors.

In addition, expression of this gene is upregulated in resting dermal fibroblast CCD1070 (CT=35.7) by treatment with TNF alpha (CT=32.1). Expression of this gene in stimulated dermal fibroblasts suggests that this gene may be important in the treatment of psoriasis.

The CG57656-02 gene encodes a protein with similarity to the Drosophila turtle protein, a member of the Ig superfamily (IgSF). The 5 Ig domains and 2 fibronectin domains in this membrane protein are characteristic of several types of cell surface receptors and cell adhesion molecules.

In addition to the utility in treatment of psoriasis, the expression of this gene in basophils and several types of T lymphocyte preparations suggests that antibodies or small molecules that antagonize the function of the CG57656-02 protein may be useful in reduction or elimination of the symptoms of various T cell-dependent or basophil-dependent medical conditions, such as, but not limited to, Crohn's disease, ulcerative colitis, multiple sclerosis, chronic obstructive pulmonary disease, asthma, emphysema, rheumatoid arthritis, and lupus erythematosus.

Furthermore, the extracellular domain of this protein may function to block the binding of the cognate ligand of the CG57656-02 protein on neighboring cells. This may modulate the function of T lymphocytes and basophils, and be useful as a protein therapeutic to reduce or eliminate the symptoms in the symptoms of various T cell-dependent or basophil-dependent medical conditions, such as, but not limited to, Crohn's disease, ulcerative colitis, multiple sclerosis, chronic obstructive pulmonary disease, asthma, emphysema, rheumatoid arthritis, and lupus erythematosus, or psoriasis.

Panel 4D Summary: Ag3298 Results using this probe/primer set are in excellent agreement with those obtained using Ag4482 on Panel 4.1D. Please see Panel 4.1D for a description of the potential utility of this gene in immune function.

NOV46

Expression of NOV46/CG57682-01 was assessed using the primer-probe set Ag3308, described in Table ANA. Results of the RTQ-PCR runs are shown in Tables ANB, ANC and AND.

TABLE ANA
Probe Name Ag3308

| Primers | Sequences | Length | Start Position | SEQ ID NO |
|---|---|---|---|---|
| Forward | 5′-gcaacaaattcctgaagatcag-3′ | 22 | 1153 | 398 |
| Probe | TET-5′-cctcagctgaactcaaaagccatcaa-3′-TAMRA | 26 | 1182 | 399 |
| Reverse | 5′-atcagagggaaggcaaggt-3′ | 19 | 1210 | 400 |

TABLE ANB
CNS_neurodegeneration_v1.0

| | Rel. Exp. (%) Ag3308, Run | | Rel. Exp. (%) Ag3308, Run |
|---|---|---|---|
| Tissue Name | 210143040 | Tissue Name | 210143040 |
| AD 1 Hippo | 5.0 | Control (Path) 3 Temporal | 9.9 |
| | | Ctx | |
| AD 2 Hippo | 25.2 | Control (Path) 4 Temporal | 36.6 |
| | | Ctx | |
| AD 3 Hippo | 14.6 | AD 1 Occipital Ctx | 46.3 |
| AD 4 Hippo | 4.4 | AD 2 Occipital Ctx | 0.0 |
| | | (Missing) | |
| AD 5 Hippo | 100.0 | AD 3 Occipital Ctx | 17.2 |
| AD 6 Hippo | 37.1 | AD 4 Occipital Ctx | 33.9 |
| Control 2 Hippo | 8.2 | AD 5 Occipital Ctx | 8.4 |
| Control 4 Hippo | 25.2 | AD 6 Occipital Ctx | 18.6 |
| Control (Path) 3 Hippo | 4.4 | Control 1 Occipital Ctx | 2.4 |
| AD 1 Temporal Ctx | 21.9 | Control 2 Occipital Ctx | 18.2 |
| AD 2 Temporal Ctx | 21.8 | Control 3 Occipital Ctx | 32.1 |
| AD 3 Temporal Ctx | 15.1 | Control 4 Occipital Ctx | 2.2 |
| AD 4 Temporal Ctx | 29.7 | Control (Path) 1 Occipital | 64.2 |
| | | Ctx | |
| AD 5 Inf Temporal Ctx | 70.7 | Control (Path) 2 Occipital | 22.1 |
| | | Ctx | |
| AD 5 Sup Temporal Ctx | 41.5 | Control (Path) 3 Occipital | 8.4 |
| | | Ctx | |
| AD 6 Inf Temporal Ctx | 59.5 | Control (Path) 4 Occipital | 38.2 |
| | | Ctx | |
| AD 6 Sup Temporal Ctx | 53.2 | Control 1 Parietal Ctx | 10.5 |
| Control 1 Temporal Ctx | 17.8 | Control 2 Parietal Ctx | 42.3 |
| Control 2 Temporal Ctx | 17.0 | Control 3 Parietal Ctx | 16.3 |
| Control 3 Temporal Ctx | 15.3 | Control (Path) 1 Parietal | 56.3 |
| | | Ctx | |
| Control 3 Temporal Ctx | 16.6 | Control (Path) 2 Parietal | 17.9 |
| | | Ctx | |
| Control (Path) 1 Temporal | 47.0 | Control (Path) 3 Parietal | 7.3 |
| Ctx | | Ctx | |
| Control (Path) 2 Temporal | 44.1 | Control (Path) 4 Parietal | 69.3 |
| Ctx | | Ctx | |

TABLE ANC
General_screening_panel_v1.4

| | Rel. Exp. (%) Ag3308, Run | | Rel. Exp. (%) Ag3308, Run |
|---|---|---|---|
| Tissue Name | 215648361 | Tissue Name | 215648361 |
| Adipose | 13.4 | Renal ca. TK-10 | 31.0 |
| Melanoma* Hs688(A).T | 4.6 | Bladder | 33.0 |
| Melanoma* Hs688(B).T | 4.0 | Gastric ca. (liver met.) NCI- | 57.0 |
| | | N87 | |
| Melanoma* M14 | 21.8 | Gastric ca. KATO III | 59.9 |
| Melanoma* LOXIMVI | 10.4 | Colon ca. SW-948 | 6.8 |
| Melanoma* SK-MEL-5 | 24.3 | Colon ca. SW480 | 82.9 |
| Squamous cell carcinoma | 14.5 | Colon ca.* (SW480 met) | 47.6 |
| SCC-4 | | SW620 | |
| Testis Pool | 12.6 | Colon ca. HT29 | 30.6 |
| Prostate ca.* (bone met) | 24.7 | Colon ca. HCT-116 | 42.3 |
| PC-3 | | | |
| Prostate Pool | 30.8 | Colon ca. CaCo-2 | 42.0 |
| Placenta | 6.9 | Colon cancer tissue | 8.5 |
| Uterus Pool | 9.5 | Colon ca. SW1116 | 8.7 |
| Ovarian ca. OVCAR-3 | 29.3 | Colon ca. Colo-205 | 13.2 |
| Ovarian ca. SK-OV-3 | 37.4 | Colon ca. SW-48 | 4.0 |
| Ovarian ca. OVCAR-4 | 4.8 | Colon Pool | 31.9 |
| Ovarian ca. OVCAR-5 | 59.9 | Small Intestine Pool | 60.3 |
| Ovarian ca. IGROV-1 | 7.5 | Stomach Pool | 34.2 |
| Ovarian ca. OVCAR-8 | 19.6 | Bone Marrow Pool | 39.0 |
| Ovary | 25.7 | Fetal Heart | 17.2 |
| Breast ca. MCF-7 | 43.2 | Heart Pool | 9.5 |
| Breast ca. MDA-MB-231 | 47.3 | Lymph Node Pool | 34.4 |
| Breast ca. BT 549 | 32.1 | Fetal Skeletal Muscle | 13.2 |
| Breast ca. T47D | 59.5 | Skeletal Muscle Pool | 23.0 |
| Breast ca. MDA-N | 27.5 | Spleen Pool | 22.4 |
| Breast Pool | 52.9 | Thymus Pool | 18.3 |
| Trachea | 32.3 | CNS cancer (glio/astro) U87- | 40.3 |
| | | MG | |
| Lung | 16.5 | CNS cancer (glio/astro) U- | 61.1 |
| | | 118-MG | |
| Fetal Lung | 52.5 | CNS cancer (neuro; met) SK- | 27.5 |
| | | N-AS | |
| Lung ca. NCI-N417 | 11.4 | CNS cancer (astro) SF-539 | 19.5 |
| Lung ca. LX-1 | 47.3 | CNS cancer (astro) SNB-75 | 65.1 |
| Lung ca. NCI-H146 | 7.6 | CNS cancer (glio) SNB-19 | 15.0 |
| Lung ca. SHP-77 | 69.3 | CNS cancer (glio) SF-295 | 92.7 |
| Lung ca. A549 | 19.2 | Brain (Amygdala) Pool | 9.8 |
| Lung ca. NCI-H526 | 11.7 | Brain (cerebellum) | 6.2 |
| Lung ca. NCI-H23 | 81.8 | Brain (fetal) | 38.4 |
| Lung ca. NCI-H460 | 25.7 | Brain (Hippocampus) Pool | 12.0 |
| Lung ca. HOP-62 | 26.2 | Cerebral Cortex Pool | 13.0 |
| Lung ca. NCI-H522 | 29.7 | Brain (Substantia nigra) Pool | 4.6 |
| Liver | 0.0 | Brain (Thalamus) Pool | 19.9 |
| Fetal Liver | 14.8 | Brain (whole) | 8.9 |
| Liver ca. HepG2 | 12.6 | Spinal Cord Pool | 9.8 |
| Kidney Pool | 100.0 | Adrenal Gland | 20.2 |
| Fetal Kidney | 47.0 | Pituitary gland Pool | 7.3 |
| Renal ca. 786-0 | 24.1 | Salivary Gland | 12.0 |
| Renal ca. A498 | 9.3 | Thyroid (female) | 4.3 |
| Renal ca. ACHN | 7.3 | Pancreatic ca. CAPAN2 | 42.6 |
| Renal ca. UO-31 | 13.9 | Pancreas Pool | 62.0 |

TABLE AND
Panel 4D

| | Rel. Exp. (%) Ag3308, Run | | Rel. Exp. (%) Ag3308, Run |
|---|---|---|---|
| Tissue name | 164335486 | Tissue Name | 164335486 |
| Secondary Th1 act | 17.7 | HUVEC IL-1beta | 6.0 |
| Secondary Th2 act | 29.3 | HUVEC IFN gamma | 16.5 |
| Secondary Tr1 act | 22.5 | HUVEC TNF alpha + IFN gamma | 9.2 |
| Secondary Th1 rest | 10.3 | HUVEC TNF alpha + IL4 | 15.9 |
| Secondary Th2 rest | 7.4 | HUVEC IL-11 | 9.9 |
| Secondary Tr1 rest | 20.3 | Lung Microvascular EC none | 9.2 |
| Primary Th1 act | 9.2 | Lung Microvascular EC TNF alpha + | 9.9 |
| | | IL-1beta | |
| Primary Th2 act | 16.6 | Microvascular Dermal EC none | 5.9 |
| Primary Tr1 act | 23.7 | Microsvasular Dermal EC | 11.7 |
| | | TNF alpha + IL-1beta | |
| Primary Th1 rest | 82.4 | Bronchial epithelium TNF alpha + | 19.1 |
| | | IL1beta | |
| Primary Th2 rest | 50.7 | Small airway epithelium none | 7.9 |
| Primary Tr1 rest | 45.1 | Small airway epithelium TNF alpha + | 40.9 |
| | | IL-1beta | |
| CD45RA CD4 lymphocyte | 19.9 | Cornery artery SMC rest | 7.5 |
| act | | | |
| CD45RO CD4 lymphocyte | 20.0 | Coronery artery SMC TNF alpha + | 0.7 |
| act | | IL-1beta | |
| CD8 lymphocyte act | 8.2 | Astrocytes rest | 7.6 |
| Secondary CD8 lymphocyte | 24.3 | Astrocytes TNF alpha + IL-1beta | 0.8 |
| rest | | | |
| Secondary CD8 lymphocyte | 18.3 | KU-812 (Basophil) rest | 37.6 |
| act | | | |
| CD4 lymphocyte none | 9.8 | KU-812 (Basophil) | 75.8 |
| | | PMA/ionomycin | |
| 2ry Th1/Th2/Tr1_anti-CD95 | 28.3 | CCD1106 (Keratinocytes) none | 13.1 |
| CH11 | | | |
| LAK cells rest | 36.1 | CCD1106 (Keratinocytes) | 4.3 |
| | | TNF alpha + IL-1beta | |
| LAK cells IL-2 | 38.7 | Liver cirrhosis | 9.0 |
| LAK cells IL-2 + IL-12 | 36.3 | Lupus kidney | 10.9 |
| LAK cells IL-2 + IFN gamma | 65.5 | NCI-H292 none | 36.1 |
| LAK cells IL-2 + IL-18 | 30.1 | NCI-H292 IL-4 | 56.3 |
| LAK cells PMA/ionomycin | 11.0 | NCI-H292 IL-9 | 73.2 |
| NK Cells IL-2 rest | 25.0 | NCI-H292 IL-13 | 28.9 |
| Two Way MLR 3 day | 31.9 | NCI-H292 IFN gamma | 31.2 |
| Two Way MLR 5 day | 19.9 | HPAEC none | 15.7 |
| Two Way MLR 7 day | 17.9 | HPAEC TNF alpha + IL-1beta | 10.1 |
| PBMC rest | 28.9 | Lung fibroblast none | 5.2 |
| PBMC PWM | 58.2 | Lung fibroblast TNF alpha + IL- | 6.5 |
| | | 1beta | |
| PBMC PHA-L | 30.4 | Lung fibroblast IL-4 | 3.7 |
| Ramos (B cell) none | 53.2 | Lung fibroblast IL-9 | 11.8 |
| Ramos (B cell) ionomycin | 71.2 | Lung fibroblast IL-13 | 4.0 |
| B lymphocytes PWM | 100.0 | Lung fibroblast IFN gamma | 17.3 |
| B lymphocytes CD40L and | 52.5 | Dermal fibroblast CCD1070 rest | 52.1 |
| IL-4 | | | |
| EOL-1 dbcAMP | 17.8 | Dermal fibroblast CCD1070 TNF | 78.5 |
| | | alpha | |
| EOL-1 dbcAMP | 19.6 | Dermal fibroblast CCD1070 IL- | 11.9 |
| PMA/ionomycin | | 1beta | |
| Dendritic cells none | 10.2 | Dermal fibroblast IFN gamma | 4.3 |
| Dendritic cells LPS | 11.0 | Dermal fibroblast IL-4 | 12.0 |
| Dendritic cells anti-CD40 | 15.1 | IBD Colitis 2 | 2.0 |
| Monocytes rest | 20.9 | IBD Crohn's | 2.4 |
| Monocytes LPS | 7.2 | Colon | 55.9 |
| Macrophages rest | 4.7 | Lung | 3.7 |
| Macrophages LPS | 0.0 | Thymus | 60.3 |
| HUVEC none | 16.4 | Kidney | 45.4 |
| HUVEC starved | 37.6 | | |

CNS_neurodegeneration_v1.0 Summary: Ag3308 This panel confirms th e expression of CG57678-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3308 Expression of CG57682-01 gene is highest in kidney (CT=31.5). However, this gene is expressed at low to moderate levels across the majority of samples on this panel, suggesting that it may play a general role in cellular function and proliferation in a variety of cell types. The CG57682-01 gene encodes a protein with homology to G2/mitotic-specific cyclin B2.

This gene is expressed at low levels in several regions of the brain, including hippocampus, amygdala, cerebral cortex, thalamus, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at low levels in adrenal gland, pancreas, adipose, heart and skeletal muscle. Therefore, therapeutic modulation of the activity of this gene or its protein product may prove useful in the treatment of endocrine/metabolically related diseases, such as obesity and diabetes.

In addition, this gene is differentially expressed in fetal (CT=34) as compared to adult liver (CT=40) and may be useful for the identification of the fetal phenotype in this tissue.

Panel 4D Summary: Ag3308 CG57682-01 gene is expressed at highest levels in stimulated B lymphocytes (CT=31.7). This gene is expressed at low levels in a wide range of cell types of significance in the immune response in health and disease. These cells include members of the T-cell, B-cell, endothelial cell, basophil, eosinophil, and peripheral blood mononuclear cell family, as well as epithelial and fibroblast cell types from lung and skin, and normal tissues represented by colon, thymus and kidney. This ubiquitous pattern of expression suggests that this gene product may be involved in homeostatic processes for these and other cell types and tissues.

This pattern is in agreement with the expression profile in General_screening_panel_v1.4 and also suggests a role for the gene product in cell survival and proliferation.

Therefore, therapeutic modulation of the activity of this gene or its protein product may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

NOV48

Expression of NOV48/CG57713-01 was assessed using the primer-probe set Ag3313, described in Table AOA. Results of the RTQ-PCR runs are shown in Tables AOB, AOC, AOD and AOE.

TABLE AOA
Probe Name Ag3313

| | | | | |
|---|---|---|---|---|
| Primers | Sequences | Length | Start Position | SEQ ID NO |

| | | | | |
|---|---|---|---|---|
| Forward | 5′-agcatgtacatgtttcctttgc-3′ | 22 | 1162 | 401 |
| Probe | TET-5′-atgcacttttccagtctgcagaagcg-3′-TAMRA | 26 | 1187 | 402 |
| Reverse | 5′-tgcaacatttcacttccataca-3′ | 22 | 1238 | 403 |

TABLE AOB
CNS_neurodegeneration_v1.0

| | Rel. Exp. (%) Ag3313, Run | | Rel. Exp. (%) Ag3313, Run |
|---|---|---|---|
| Tissue Name | 210143317 | Tissue Name | 210143317 |
| AD 1 Hippo | 5.7 | Control (Path) 3 Temporal | 1.4 |
| | | Ctx | |
| AD 2 Hippo | 13.7 | Control (Path) 4 Temporal | 32.8 |
| | | Ctx | |
| AD 3 Hippo | 1.0 | AD 1 Occipital Ctx | 16.2 |
| AD 4 Hippo | 4.0 | AD 2 Occipital Ctx | 0.0 |
| | | (Missing) | |
| AD 5 Hippo | 51.1 | AD 3 Occipital Ctx | 3.1 |
| AD 6 hippo | 0.0 | AD 4 Occipital Ctx | 39.5 |
| Control 2 Hippo | 16.5 | AD 5 Occipital Ctx | 10.9 |
| Control 4 Hippo | 3.7 | AD 6 Occipital Ctx | 63.3 |
| Control (Path) 3 Hippo | 0.7 | Control 1 Occipital Ctx | 1.4 |
| AD 1 Temporal Ctx | 6.8 | Control 2 Occipital Ctx | 33.4 |
| AD 2 Temporal Ctx | 36.6 | Control 3 Occipital Ctx | 19.5 |
| AD 3 Temporal Ctx | 1.2 | Control 4 Occipital Ctx | 2.5 |
| AD 4 Temporal Ctx | 25.3 | Control (Path) 1 Occipital | 80.7 |
| | | Ctx | |
| AD 5 Inf Temporal Ctx | 100.0 | Control (Path) 2 Occipital | 14.5 |
| | | Ctx | |
| AD 5 SupTemporal Ctx | 26.6 | Control (Path) 3 Occipital | 0.0 |
| | | Ctx | |
| AD 6 Inf Temporal Ctx | 15.4 | Control (Path) 4 Occipital | 12.6 |
| | | Ctx | |
| AD 6 Sup Temporal Ctx | 30.4 | Control 1 Parietal Ctx | 10.1 |
| Control 1 Temporal Ctx | 2.2 | Control 2 Parietal Ctx | 37.1 |
| Control 2 Temporal Ctx | 34.4 | Control 3 Parietal Ctx | 14.7 |
| Control 3 Temporal Ctx | 18.2 | Control (Path) 1 Parietal | 63.7 |
| | | Ctx | |
| Control 4 Temporal Ctx | 11.1 | Control (Path) 2 Parietal | 32.3 |
| | | Ctx | |
| Control (Path) 1 Temporal | 58.2 | Control (Path) 3 Parietal | 1.9 |
| Ctx | | Ctx | |
| Control (Path) 2 Temporal | 49.7 | Control (Path) 4 Parietal | 52.1 |
| Ctx | | Ctx | |

TABLE AOC
General_screening_panel_v1.4

| | Rel. Exp. (%) Ag3313, Run | | Rel. Exp. (%) Ag3313, Run |
|---|---|---|---|
| Tissue Name | 215648086 | Tissue Name | 215648086 |
| Adipose | 0.1 | Renal ca. TK-10 | 0.2 |
| Melanoma* Hs688(A).T | 0.1 | Bladder | 0.3 |
| Melanoma* Hs688(B) T | 0.1 | Gastric ca. (liver met.) NCI- | 0.4 |
| | | N87 | |
| Melanoma* M14 | 1.5 | Gastric ca. KATO III | 0.1 |
| Melanoma* LOXIMVI | 0.1 | Colon ca. SW-948 | 0.0 |
| Melanoma* SK-MEL-5 | 0.1 | Colon ca. SW480 | 0.2 |
| Squamous cell carcinoma | 0.3 | Colon ca.* (SW480 met) | 0.1 |
| SCC-4 | | SW620 | |
| Testis Pool | 0.6 | Colon ca. HT29 | 0.1 |
| Prostate ca.* (bone met) | 0.2 | Colon ca. HCT-116 | 30.1 |
| PC-3 | | | |
| Prostate Pool | 0.2 | Colon ca. CaCo-2 | 7.0 |
| Placenta | 0.3 | Colon cancer tissue | 0.3 |
| Uterus Pool | 0.0 | Colon ca. SW1116 | 0.1 |
| Ovarian ca. OVCAR-3 | 1.6 | Colon ca. Colo-205 | 0.1 |
| Ovarian ca. SK-OV-3 | 6.4 | Colon ca. SW-48 | 0.0 |
| Ovarian ca. OVCAR-4 | 0.4 | Colon Pool | 0.3 |
| Ovarian ca. OVCAR-5 | 0.4 | Small Intestine Pool | 0.5 |
| Ovarian ca. IGROV-1 | 1.7 | Stomach Pool | 0.5 |
| Ovarian ca. OVCAR-8 | 0.0 | Bone Marrow Pool | 0.6 |
| Ovary | 0.1 | Fetal Heart | 0.3 |
| Breast ca. MCF-7 | 0.4 | Heart Pool | 0.1 |
| Breast ca. MDA-MB-231 | 0.7 | Lymph Node Pool | 0.3 |
| Breast ca. BT 549 | 3.1 | Fetal Skeletal Muscle | 0.4 |
| Breast ca. T47D | 0.4 | Skeletal Muscle Pool | 0.1 |
| Breast ca. MDA-N | 0.0 | Spleen Pool | 0.4 |
| Breast Pool | 0.2 | Thymus Pool | 0.9 |
| Trachea | 0.5 | CNS cancer (glio/astro) U87- | 0.3 |
| | | MG | |
| Lung | 0.1 | CNS cancer (glio/astro) U- | 0.3 |
| | | 118-MG | |
| Fetal Lung | 0.5 | CNS cancer (neuro; met) SK- | 100.0 |
| | | N-AS | |
| Lung ca. NCI-N417 | 1.9 | CNS cancer (astro) SF-539 | 0.7 |
| Lung ca. LX-1 | 0.4 | CNS cancer (astro) SNB-75 | 1.0 |
| Lung ca. NCI-H146 | 3.3 | CNS cancer (glio) SNB-19 | 0.6 |
| Lung ca. SHP-77 | 1.2 | CNS cancer (glio) SF-295 | 0.5 |
| Lung ca. A549 | 13.0 | Brain (Amygdala) Pool | 4.5 |
| Lung ca. NCI-H526 | 0.4 | Brain (cerebellum) | 0.1 |
| Lung ca. NCI-H23 | 5.6 | Brain (fetal) | 7.4 |
| Lung ca. NCI-H460 | 2.9 | Brain (Hippocampus) Pool | 3.3 |
| Lung ca. HOP-62 | 1.1 | Cerebral Cortex Pool | 2.9 |
| Lung ca. NCI-H522 | 3.8 | Brain (Substantia nigra) Pool | 4.5 |
| Liver | 0.0 | Brain (Thalamus) Pool | 4.5 |
| Fetal Liver | 2.4 | Brain (whole) | 2.6 |
| Liver ca. HepG2 | 0.0 | Spinal Cord Pool | 4.3 |
| Kidney Pool | 0.1 | Adrenal Gland | 2.0 |
| Fetal Kidney | 0.7 | Pituitary gland Pool | 5.9 |
| Renal ca. 786-0 | 0.0 | Salivary Gland | 3.2 |
| Renal ca. A498 | 0.0 | Thyroid (female) | 0.3 |
| Renal ca. ACHN | 0.3 | Pancreatic ca. CAPAN2 | 0.8 |
| Renal ca. UO-31 | 0.0 | Pancreas Pool | 0.2 |

TABLE AOD
Panel 4D

| | Rel. Exp. (%) Ag3313, Run | | Rel. Exp. (%) Ag3313, Run |
|---|---|---|---|
| Tissue Name | 164682847 | Tissue Name | 164682847 |
| Secondary Th1 act | 0.2 | HUVEC IL-1beta | 0.1 |
| Secondary Th2 act | 0.2 | HUVEC IFN gamma | 0.1 |
| Secondary Tr1 act | 0.0 | HUVEC TNF alpha + IFN gamma | 0.1 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.2 | Lung Microvascular EC none | 0.1 |
| Primary Th1 act | 0.0 | Lung Microvascular EC TNF alpha + | 0.1 |
| | | IL-1beta | |
| Primary Th2 act | 0.1 | Microvascular Dermal EC none | 0.1 |
| Primary Tr1 act | 0.0 | Microsvasular Dermal EC | 0.1 |
| | | TNF alpha + IL-1beta | |
| Primary Th1 rest | 0.0 | Bronchial epithelium TNF alpha + | 0.0 |
| | | IL1beta | |
| Primary Th2 rest | 0.4 | Small airway epithelium none | 0.1 |
| Primary Tr1 rest | 0.1 | Small airway epithelium TNF alpha + | 0.4 |
| | | IL-1beta | |
| CD45RA CD4 lymphocyte | 0.3 | Coronery artery SMC rest | 0.2 |
| act | | | |
| CD45RO CD4 lymphocyte | 0.1 | Coronery artery SMC TNF alpha + | 0.0 |
| act | | IL-1beta | |
| CD8 lymphocyte act | 0.1 | Astrocytes rest | 4.9 |
| Secondary CD8 lymphocyte | 0.1 | Astrocytes TNF alpha + IL-1beta | 0.9 |
| rest | | | |
| Secondary CD8 lymphocyte | 0.3 | KU-812 (Basophil) rest | 81.2 |
| act | | | |
| CD4 lymphocyte none | 0.1 | KU-812 (Basophil) | 100.0 |
| | | PMA/ionomycin | |
| 2ry Th1/Th2/Tr1_anti-CD95 | 0.1 | CCD1106 (Keratinocytes) none | 0.3 |
| CH11 | | | |
| LAK cells rest | 0.0 | CCD1106 (Keratinocytes) | 0.1 |
| | | TNF alpha + IL-1beta | |
| LAK cells IL-2 | 0.0 | Liver cirrhosis | 0.2 |
| LAK cells IL-2 + IL-12 | 0.2 | Lupus kidney | 0.0 |
| LAK cells IL-2 + IFN gamma | 0.4 | NCI-H292 none | 0.2 |
| LAK cells IL-2 + IL-18 | 0.3 | NCI-H292 IL-4 | 0.6 |
| LAK cells PMA/ionomycin | 0.3 | NCI-H292 IL-9 | 0.1 |
| NK Cells IL-2 rest | 0.0 | NCI-H292 IL-13 | 0.0 |
| Two Way MLR 3 day | 0.2 | NCI-H292 IFN gamma | 0.2 |
| Two Way MLR 5 day | 0.0 | HPAEC none | 0.1 |
| Two Way MLR 7 day | 0.0 | HPAEC TNF alpha + IL-1beta | 0.1 |
| PBMC rest | 0.0 | Lung fibroblast none | 0.3 |
| PBMC PWM | 0.2 | Lung fibroblast TNF alpha + IL- | 0.1 |
| | | 1beta | |
| PBMC PHA-L | 0.0 | Lung fibroblast IL-4 | 0.3 |
| Ramos (B cell) none | 0.0 | Lung fibroblast IL-9 | 0.4 |
| Ramos (B cell) ionomycin | 0.0 | Lung fibroblast IL-13 | 1.1 |
| B lymphocytes PWM | 1.3 | Lung fibroblast IFN gamma | 0.3 |
| B lymphocytes CD40L and | 0.7 | Dermal fibroblast CCD1070 rest | 0.4 |
| IL-4 | | | |
| EOL-1 dbcAMP | 0.2 | Dermal fibroblast CCD1070 TNF | 0.8 |
| | | alpha | |
| EOL-1 dbcAMP | 0.6 | Dermal fibroblast CCD1070 IL- | 0.1 |
| PMA/ionomycin | | 1beta | |
| Dendritic cells none | 0.1 | Dermal fibroblast IFN gamma | 0.1 |
| Dendritic cells LPS | 0.1 | Dermal fibroblast IL-4 | 0.1 |
| Dendritic cells anti-CD40 | 0.1 | IBD Colitis 2 | 0.1 |
| Monocytes rest | 0.0 | IBD Crohn's | 0.3 |
| Monocytes LPS | 0.3 | Colon | 2.7 |
| Macrophages rest | 0.0 | Lung | 1.9 |
| Macrophages LPS | 0.1 | Thymus | 0.5 |
| HUVEC none | 0.0 | Kidney | 1.2 |
| HUVEC starved | 0.1 | | |

TABLE AOE
Panel 5 Islet

| | Rel. Exp. (%) Ag3313, | | Rel. Exp. (%) Ag3313, |
|---|---|---|---|
| Tissue Name | Run 242385176 | Tissue Name | Run 242385176 |
| 97457_Patient-02go_adipose | 0.0 | 94709_Donor 2 AM - A_adipose | 0.0 |
| 97476_Patient-07sk_skeletal | 0.0 | 94710_Donor 2 AM - B_adipose | 0.0 |
| muscle | | | |
| 97477_Patient-07ut_uterus | 0.0 | 94711_Donor 2 AM - C_adipose | 0.0 |
| 97478_Patient-07pl_placenta | 9.2 | 94712_Donor 2 AD - A_adipose | 22.2 |
| 99167_Bayer Patient 1 | 0.0 | 94713_Donor 2 AD - B_adipose | 0.0 |
| 97482_Patient-08ut_uterus | 0.0 | 94714_Donor 2 AD - C_adipose | 0.0 |
| 97483_Patient-08pl_placenta | 7.8 | 94742_Donor 3 U - A_Mesenchymal | 0.0 |
| | | Stem Cells | |
| 97486_Patient-09sk_skeletal | 0.0 | 94743_Donor 3 U - B_Mesenchymal | 0.0 |
| muscle | | Stem Cells | |
| 97487_Patient-09ut_uterus | 0.0 | 94730_Donor 3 AM - A_adipose | 0.0 |
| 97488_Patient-09pl_placenta | 0.0 | 94731_Donor 3 AM - B_adipose | 0.0 |
| 97492_Patient-10ut_uterus | 0.0 | 94732_Donor 3 AM - C_adipose | 0.0 |
| 97493_Patient-10pl_placenta | 7.3 | 94733_Donor 3 AD - A_adipose | 13.5 |
| 97495_Patient-11go_adipose | 0.0 | 94734_Donor 3 AD - B_adipose | 0.0 |
| 97496_Patient-11sk_skeletal | 0.0 | 94735_Donor 3 AD - C_adipose | 0.0 |
| muscle | | | |
| 97497_Patient-11ut_uterus | 0.0 | 77138_Liver_HepG2untreated | 0.0 |
| 97498_Patient-11pl_placenta | 0.0 | 73556_Heart_Cardiac stromal cells | 0.0 |
| | | (primary) | |
| 97500_Patient-12go_adipose | 6.4 | 81735_Small Intestine | 100.0 |
| 97501_Patient-12sk_skeletal | 7.9 | 72409_Kidney_Proximal Convoluted | 0.0 |
| muscle | | Tubule | |
| 97502_Patient-12ut_uterus | 0.0 | 82685_Small intestine_Duodenum | 0.0 |
| 97503_Patient-12pl_placenta | 0.0 | 90650_Adrenal_Adrenocortical | 0.0 |
| | | adenoma | |
| 94721_Donor 2U - | 0.0 | 72410_Kidney_HRCE | 9.5 |
| A_Mesenchymal Stem Cells | | | |
| 94722_Donor 2 U - | 7.2 | 72411_Kidney_HRE | 0.0 |
| B_Mesenchymal Stem Cells | | | |
| 94723_Donor 2 U - | 0.0 | 73139_Uterus_Uterine smooth muscle | 0.0 |
| C_Mesenchymal Stem Cells | | cells | |

CNS_neurodegeneration_v1.0 Summary: Ag3313 This panel confirms the expression of CG57713-01 gene at low to moderate levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3313 Expression of the CG57713-01 gene is highest in CNS cancer cell line SK-N-AS (CT=26.1). Expression of this gene also appears to be upregulated in a number of lung cancer cell lines and ovarian cancer cell lines when compared to the normal tissues. Therefore, therapeutic modulation of the activity of this gene or its protein product may be of use in the treatment of lung or ovarian cancer.

In addition, this gene is expressed at moderate levels in most regions of the central nervous system examined, including amygdala, hippocampus, cerebral cortex, substantia nigra, thalamus and spinal cord. The CG57713-01 gene encodes a protein with homology to sodium/bile acid cotransporters. Changes in expression of sodium/bile acid cotransporters have been found to be associated with methamphetamine (METH)-induced dopamine (DA) neurotoxicity (ref. 1). In addition, based on the expression of the CG57713-01 gene in the brain, this gene may play a role in central nervous system disorders such as Alzheimer's disease, Parkinson's disease, epilepsy, multiple sclerosis, schizophrenia and depression.

Among tissues with metabolic or endocrine function, this gene is expressed at moderate levels in adrenal gland and pituitary gland and at low levels in pancreas, thyroid, fetal heart, fetal skeletal muscle, and fetal liver. Therefore, therapeutic modulation of the activity of this gene may prove useful in the treatment of endocrine/metabolically related diseases, such as obesity and diabetes. Interestingly, expression of this gene is much higher in fetal liver (CT=31.5) than in adult liver (CT=40), suggesting that expression of this gene can be used to distinguish these two tissues.

References:

1. Xie T, Tong L, Barrett T, Yuan J, Hatzidimitriou G, McCann U D, Becker K G, Donovan D M, Ricaurte G A. Changes in gene expression linked to methamphetamine induced dopaminergic neurotoxicity. J Neurosci Jan. 1, 2002;22(1):274-83

The purpose of these studies was to examine the role of gene expression in methamphetamine (METH)-induced dopamine (DA) neurotoxicity. First, the effects of the mRNA synthesis inhibitor, actinomycin-D, and the protein synthesis inhibitor, cycloheximide, were examined. Both agents afforded complete protection against METH-induced DA neurotoxicity and did so independently of effects on core temperature, DA transporter function, or METH brain levels, suggesting that gene transcription and mRNA translation play a role in METH neurotoxicity. Next, microarray technology, in combination with an experimental approach designed to facilitate recognition of relevant gene expression patterns, was used to identify gene products linked to METH-induced DA neurotoxicity. This led to the identification of several genes in the ventral midbrain associated with the neurotoxic process, including genes for energy metabolism [cytochrome c oxidase subunit 1 (COX1), reduced nicotinamide adenine dinucleotide ubiquinone oxidoreductase chain 2, and phosphoglycerate mutase B], ion regulation (members of sodium/hydrogen exchanger and sodium/bile acid cotransporter family), signal transduction (adenylyl cyclase III), and cell differentiation and degeneration (N-myc downstream-regulated gene 3 and tau protein). Of these differentially expressed genes, we elected to further examine the increase in COX1 expression, because of data implicating energy utilization in METH neurotoxicity and the known role of COX1 in energy metabolism. On the basis of time course studies, Northern blot analyses, in situ hybridization results, and temperature studies, we now report that increased COX1 expression in the ventral midbrain is linked to METH induced DA neuronal injury. The precise role of COX1 and other genes in METH neurotoxicity remains to be elucidated.

PMID: 11756511

Panel 4D Summary: Ag3313 The CG57713-01 transcript is expressed at highest levels in both PMA/ionomycin-treated KU-812 basophil cell line and in untreated KU-812 cells (CTs=28). Therefore, expression of this gene can be used to distinguish basophils from the other samples on this panel. In addition, small molecule therapies designed with the protein encoded for by this gene could block or inhibit inflammation or tissue damage due to basophil activation in response to asthma, allergies, hypersensitivity reactions, psoriasis, and viral infections.

In addition, expression of this gene is higher in normal colon (CT=32.9) than in colon samples from patients with IBD Crohn's (CT=36) or IBD colitis (CT=37). The CG57713-01 gene encodes a protein with homology to sodium/bile acid cotransporters. Defects in the sodium/bile acid cotransporter are one of the causative agents for Crohn's disease (Ref 1). Thus, therapeutic modulation of this novel cotransporter may be of use in the treatment of Crohn's disease.

References:

1. Wong M H, Oelkers P, Dawson P A. (1995) Identification of a mutation in the ileal sodium-dependent bile acid transporter gene that abolishes transport activity. J. Biol. Chem. 270(45):27228-34.

The ileal Na+/bile acid cotransporter plays a critical role in the reabsorption of bile acids from the small intestine. In the course of cloning and characterizing the human ileal Na+/bile acid cotransporter cDNA, a dysfunctional isoform was identified in a patient diagnosed with Crohn's disease. Expression studies using hamster-human ileal Na+/bile acid cotransporter cDNA chimeras narrowed the location of the defect to the carboxyl-terminal 94 amino acids. Comparison of the sequence of the dysfunctional isoform to that of a wild-type human ileal Na+/bile acid cotransporter genomic clone revealed a single C to T transition resulting in a proline to serine substitution at amino acid position 290. The inheritance of this mutation in the proband's family was confirmed by single-stranded conformation polymorphism analysis and DNA sequencing. In transfected COS-1 cells, the single amino acid change abolished taurocholate transport activity but did not alter the transporter's synthesis or subcellular distribution. This dysfunctional mutation represents the first known molecular defect in a human sodium-dependent bile acid transporter.

PMID: 7592981

Panel 5 Islet Summary: Ag3313 A low level of expression of the CG57713-01 gene is seen in a sample derived from small intestine (CT=34), which is confirmed by the expression observed in panel 1.4. Defects in the sodium/bile acid cotransporter are one of the causative agent for Crohn's disease (Ref 1). Thus, therapeutic modulation of this novel cotransporter may be of use in the treatment of Crohn's disease.

References:

1. Wong M H, Oelkers P, Dawson P A. (1995) Identification of a mutation in the ileal sodium-dependent bile acid transporter gene that abolishes transport activity. J. Biol. Chem. 270(45):27228-34.

The ileal Na+/bile acid cotransporter plays a critical role in the reabsorption of bile acids from the small intestine. In the course of cloning and characterizing the human ileal Na+/bile acid cotransporter cDNA, a dysfunctional isoform was identified in a patient diagnosed with Crohn's disease. Expression studies using hamster-human ileal Na+/bile acid cotransporter cDNA chimeras narrowed the location of the defect to the carboxyl-terminal 94 amino acids. Comparison of the sequence of the dysfunctional isoform to that of a wild-type human ileal Na+/bile acid cotransporter genomic clone revealed a single C to T transition resulting in a proline to serine substitution at amino acid position 290. The inheritance of this mutation in the proband's family was confirmed by single-stranded conformation polymorphism analysis and DNA sequencing. In transfected COS-1 cells, the single amino acid change abolished taurocholate transport activity but did not alter the transporter's synthesis or subcellular distribution. This dysfunctional mutation represents the first known molecular defect in a human sodium-dependent bile acid transporter.

PMID: 7592981

NOV49

Expression of NOV49/CG57721-01 was assessed using the primer-probe set Ag3315, described in Table APA. Results of the RTQ-PCR runs are shown in Tables APB, APC and APD.

TABLE APA
Probe Name Ag3315

| Primers | Sequences | Length | Start Position | SEQ ID NO |
|---|---|---|---|---|
| Forward | 5′-atctacctctccgagtccttca-3′ | 22 | 652 | 404 |
| Probe | TET-5′-ctgcagatcctgatttcggtgctcaa-3′-TAMRA | 26 | 700 | 405 |
| Reverse | 5′-gatggtcagtccgaagatgtac-3′ | 22 | 726 | 406 |

TABLE APB
CNS_neurodegeneration_v1.0

| | Rel. Exp. (%) Ag3315, Run | | Rel. Exp. (%) Ag3315, Run |
|---|---|---|---|
| Tissue Name | 210138297 | Tissue Name | 210138297 |
| AD 1 Hippo | 17.1 | Control (Path) 3 Temporal | 4.9 |
| | | Ctx | |
| AD 2 Hippo | 0.0 | Control (Path) 4 Temporal | 13.5 |
| | | Ctx | |
| AD 3 Hippo | 6.4 | AD 1 Occipital Ctx | 7.0 |
| AD 4 Hippo | 37.6 | AD 2 Occipital Ctx | 0.0 |
| | | (Missing) | |
| AD 5 hippo | 12.7 | AD 3 Occipital Ctx | 0.0 |
| AD 6 Hippo | 92.7 | AD 4 Occipital Ctx | 17.1 |
| Control 2 Hippo | 0.0 | AD 5 Occipital Ctx | 31.6 |
| Control 4 Hippo | 18.3 | AD 6 Occipital Ctx | 11.6 |
| Control (Path) 3 Hippo | 0.0 | Control 1 Occipital Ctx | 0.0 |
| AD 1 Temporal Ctx | 0.0 | Control 2 Occipital Ctx | 18.9 |
| AD 2 Temporal Ctx | 0.0 | Control 3 Occipital Ctx | 0.0 |
| AD 3 Temporal Ctx | 0.0 | Control 4 Occipital Ctx | 12.2 |
| AD 4 Temporal Ctx | 29.5 | Control (Path) 1 Occipital | 40.6 |
| | | Ctx | |
| AD 5 Inf Temporal Ctx | 11.7 | Control (Path) 2 Occipital | 9.3 |
| | | Ctx | |
| AD 5 SupTemporal Ctx | 30.6 | Control (Path) 3 Occipital | 5.2 |
| | | Ctx | |
| AD 6 Inf Temporal Ctx | 80.7 | Control (Path) 4 Occipital | 19.2 |
| | | Ctx | |
| AD 6 Sup Temporal Ctx | 100.0 | Control 1 Parietal Ctx | 0.0 |
| Control 1 Temporal Ctx | 0.0 | Control 2 Parietal Ctx | 29.7 |
| Control 2 Temporal Ctx | 5.5 | Control 3 Parietal Ctx | 14.4 |
| Control 3 Temporal Ctx | 5.0 | Control (Path) 1 Parietal | 11.5 |
| | | Ctx | |
| Control 4 Temporal Ctx | 13.8 | Control (Path) 2 Parietal | 5.5 |
| | | Ctx | |
| Control (Path) 1 Temporal | 31.4 | Control (Path) 3 Parietal | 0.0 |
| Ctx | | Ctx | |
| Control (Path) 2 Temporal | 24.1 | Control (Path) 4 Parietal | 11.3 |
| Ctx | | Ctx | |

TABLE APC
General_screening_panel_v1.4

| | Rel. Exp. (%) Ag3315, Run | | Rel. Exp. (%) Ag3315, Run |
|---|---|---|---|
| Tissue Name | 215678587 | Tissue Name | 215678587 |
| Adipose | 0.2 | Renal ca. TK-10 | 0.3 |
| Melanoma* Hs688(A).T | 0.0 | Bladder | 2.2 |
| Melanoma* Hs688(B).T | 0.0 | Gastric ca. (liver met.) NCI- | 12.7 |
| | | N87 | |
| Melanoma* M14 | 0.0 | Gastric ca. KATO III | 100.0 |
| Melanoma* LOXIMVI | 0.1 | Colon ca. SW-948 | 0.0 |
| Melanoma* SK-MEL-5 | 0.0 | Colon ca. SW480 | 7.1 |
| Squamous cell carcinoma | 0.1 | Colon ca.* (SW480 met) | 0.0 |
| SCC-4 | | SW620 | |
| Testis Pool | 0.1 | Colon ca. HT29 | 0.9 |
| Prostate ca.* (bone met) | 0.0 | Colon ca. HCT-116 | 0.0 |
| PC-3 | | | |
| Prostate Pool | 0.0 | Colon ca. CaCo-2 | 0.1 |
| Placenta | 0.0 | Colon cancer tissue | 0.4 |
| Uterus Pool | 0.1 | Colon ca. SW1116 | 0.0 |
| Ovarian ca. OVCAR-3 | 0.4 | Colon ca. Colo-205 | 13.3 |
| Ovarian ca. SK-OV-3 | 0.2 | Colon ca. SW-48 | 0.0 |
| Ovarian ca. OVCAR-4 | 0.2 | Colon Pool | 0.1 |
| Ovarian ca. OVCAR-5 | 6.6 | Small Intestine Pool | 0.2 |
| Ovarian ca. IGROV-1 | 3.2 | Stomach Pool | 6.7 |
| Ovarian ca. OVCAR-8 | 0.1 | Bone Marrow Pool | 0.0 |
| Ovary | 0.9 | Fetal Heart | 0.1 |
| Breast ca. MCF-7 | 0.2 | Heart Pool | 0.1 |
| Breast ca. MDA-MB-231 | 0.2 | Lymph Node Pool | 0.2 |
| Breast ca. BT 549 | 0.2 | Fetal Skeletal Muscle | 0.0 |
| Breast ca. T47D | 7.3 | Skeletal Muscle Pool | 0.4 |
| Breast ca. MDA-N | 0.0 | Spleen Pool | 0.0 |
| Breast Pool | 0.0 | Thymus pool | 0.1 |
| Trachea | 3.3 | CNS cancer (glio/astro) U87- | 0.2 |
| | | MG | |
| Lung | 0.1 | CNS cancer (glio/astro) U- | 0.0 |
| | | 118-MG | |
| Fetal Lung | 6.0 | CNS cancer (neuro; met) SK- | 0.0 |
| | | N-AS | |
| Lung ca. NCI-N417 | 0.0 | CNS cancer (astro) SF-539 | 0.1 |
| Lung ca. LX-1 | 0.6 | CNS cancer (astro) SNB-75 | 0.2 |
| Lung ca. NCI-H146 | 0.0 | CNS cancer (glio) SNB-19 | 2.6 |
| Lung ca. SHP-77 | 0.0 | CNS cancer (glio) SF-295 | 0.2 |
| Lung ca. A549 | 1.4 | Brain (Amygdala) Pool | 0.0 |
| Lung ca. NCI-H526 | 0.1 | Brain (cerebellum) | 0.0 |
| Lung ca. NCI-H23 | 0.0 | Brain (fetal) | 0.2 |
| Lung ca. NCI-H460 | 0.1 | Brain (Hippocampus) Pool | 0.0 |
| Lung ca. HOP-62 | 0.3 | Cerebral Cortex Pool | 0.1 |
| Lung ca. NCI-H522 | 0.0 | Brain (Substantia nigra) Pool | 0.2 |
| Liver | 0.0 | Brain (Thalamus) Pool | 0.1 |
| Fetal Liver | 0.1 | Brain (whole) | 0.1 |
| Liver ca. HepG2 | 0.5 | Spinal Cord Pool | 0.3 |
| Kidney Pool | 0.2 | Adrenal Gland | 0.0 |
| Fetal Kidney | 0.1 | Pituitary gland Pool | 0.1 |
| Renal ca. 786-0 | 0.1 | Salivary Gland | 79.6 |
| Renal ca. A498 | 0.1 | Thyroid (female) | 1.5 |
| Renal ca. ACHN | 0.1 | Pancreatic ca. CAPAN2 | 7.2 |
| Renal ca. UO-31 | 0.1 | Pancreas Pool | 0.7 |

TABLE APD
Panel 4D

| | Rel. Exp. (%) Ag3315, Run | | Rel. Exp. (%) Ag3315, Run |
|---|---|---|---|
| Tissue Name | 164683047 | Tissue Name | 164683047 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 1.3 |
| Secondary Th2 rest | 1.4 | HUVEC IL-11 | 3.8 |
| Secondary Tr1 rest | 1.5 | Lung Microvascular EC none | 0.0 |
| Primary Th1 act | 0.0 | Lung Microvascular EC TNF alpha + | 0.0 |
| | | IL-1beta | |
| Primary Th2 act | 0.0 | Microvascular Dermal EC none | 2.7 |
| Primary Tr1 act | 0.0 | Microvascular Dermal EC | 2.9 |
| | | TNF alpha + IL-1beta | |
| Primary Th1 rest | 0.0 | Bronchial epithelium TNF alpha + | 2.8 |
| | | IL1beta | |
| Primary Th2 rest | 0.0 | Small airway epithelium none | 4.1 |
| Primary Tr1 rest | 0.0 | Small airway epithelium TNF alpha + | 100.0 |
| | | IL-1beta | |
| CD45RA CD4 lymphocyte | 0.0 | Coronery artery SMC rest | 1.4 |
| act | | | |
| CD45RO CD4 lymphocyte | 0.0 | Coronery artery SMC TNF alpha + | 0.0 |
| act | | IL-1beta | |
| CD8 lymphocyte act | 0.0 | Astrocytes rest | 0.0 |
| Secondary CD8 lymphocyte | 0.0 | Astrocytes TNF alpha + IL-1beta | 0.0 |
| rest | | | |
| Secondary CD8 lymphocyte | 1.6 | KU-812 (Basophil) rest | 2.8 |
| act | | | |
| CD4 lymphocyte none | 1.7 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 3.1 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 1.5 |
| LAK cells PMA/ionomycin | 1.4 | NCI-H292 IL-9 | 2.1 |
| NK Cells IL-2 rest | 0.0 | NCI-H292 IL-13 | 0.0 |
| Two Way MLR 3 day | 0.0 | NCI-H292 IFN gamma | 1.5 |
| Two Way MLR 5 day | 0.0 | HPAEC none | 2.6 |
| Two Way MLR 7 day | 0.0 | HPAEC TNF alpha + IL-1beta | 1.6 |
| PBMC rest | 0.0 | Lung fibroblast none | 0.0 |
| PBMC PWM | 1.5 | Lung fibroblast TNF alpha + IL- | 0.0 |
| | | 1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 3.3 | Lung fibroblast IL-13 | 0.0 |
| B lymphocytes PWM | 1.5 | Lung fibroblast IFN gamma | 0.0 |
| B lymphocytes CD40L and | 0.0 | Dermal fibroblast CCD1070 rest | 0.0 |
| IL-4 | | | |
| EOL-1 dbcAMP | 0.0 | Dermal fibroblast CCD1070 TNF | 0.0 |
| | | alpha | |
| EOL-1 dbcAMP | 1.4 | Dermal fibroblast CCD1070 IL- | 0.0 |
| PMA/ionomycin | | 1beta | |
| Dendritic cells none | 0.0 | Dermal fibroblast IFN gamma | 0.0 |
| Dendritic cells LPS | 3.1 | Dermal fibroblast IL-4 | 0.9 |
| Dendritic cells anti-CD40 | 0.0 | IBD Colitis 2 | 0.9 |
| Monocytes rest | 0.0 | IBD Crohn's | 0.0 |
| Monocytes LPS | 3.2 | Colon | 1.4 |
| Macrophages rest | 0.0 | Lung | 39.0 |
| Macrophages LPS | 1.9 | Thymus | 5.7 |
| HUVEC none | 0.0 | Kidney | 6.9 |
| HUVEC starved | 6.7 | | |

CNS_neurodegeneration_v1.0 Summary: Ag3315 This panel confirms the expression of the CG57721-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3315 Expression of the CG57721-01 gene is highest in gastric cancer cell line KATO III and in salivary gland (CTs=25). Therefore, expression of this gene can be used to distinguish these samples from the other samples on this panel. Interestingly, this gene is expressed at much higher levels in fetal lung (CT=29.3) than in adult lung (CT=35.1), suggesting that expression of this gene may be used to distinguish fetal from adult lung.

In addition, this gene is expressed at low levels in some regions of the central nervous system, including cerebral cortex, substantia nigra, and spinal cord. Therefore, this gene may play a role in central nervous system disorders such as Alzheimer's disease, Parkinson's disease, epilepsy, multiple sclerosis, schizophrenia and depression. The CG57721-01 gene encodes a protein with similarity to the mouse prestin gene, a putative anion transporter protein. Prestin has been demonstrated to act as a motor protein of cochlear outer hair cells and presumably plays an important role in hearing (Ref. 1, 2). Based on the similarity of the CG57721-01 gene to prestin, therapeutic modulation of the activity of this gene or its protein product may be of benefit in the treatment of deafness.

Finally, among tissues with metabolic or endocrine function, this gene is expressed at low levels in pancreas, thyroid, skeletal muscle, and adipose. Therefore, therapeutic modulation of the activity of this gene may prove useful in the treatment of endocrine/metabolically related diseases, such as obesity and diabetes. This gene is expressed in the thyroid and is also similar to the pendrin gene. Mutatations in the pendrin gene are known to cause the human disease Pendred syndrome (Ref. 3). Therefore, therapeutic modulatin of this gene may be useful in treatment of Pendred syndrome and related disorders.

References:

1. Zheng J, Shen W, He D Z, Long K B, Madison L D, Dallos P. (2000) Prestin is the motor protein of cochlear outer hair cells. Nature 405(6783):149-55

The outer and inner hair cells of the mammalian cochlea perform different functions. In response to changes in membrane potential, the cylindrical outer hair cell rapidly alters its length and stiffness. These mechanical changes, driven by putative molecular motors, are assumed to produce amplification of vibrations in the cochlea that are transduced by inner hair cells. Here we have identified an abundant complementary DNA from a gene, designated Prestin, which is specifically expressed in outer hair cells. Regions of the encoded protein show moderate sequence similarity to pendrin and related sulphate/anion transport proteins. Voltage-induced shape changes can be elicited in cultured human kidney cells that express prestin. The mechanical response of outer hair cells to voltage change is accompanied by a ‘gating current’, which is manifested as nonlinear capacitance. We also demonstrate this nonlinear capacitance in transfected kidney cells. We conclude that prestin is the motor protein of the cochlear outer hair cell.

PMID: 10821263

2. Peter Dallos & Bernd Fakler (2002) Prestin, a new type of motor protein. Nature Reviews Molecular Cell Biology 3, 104-111.

Prestin, a transmembrane protein found in the outer hair cells of the cochlea, represents a new type of molecular motor, which is likely to be of great interest to molecular cell biologists. In contrast to enzymatic-activity-based motors, prestin is a direct voltage-to-force converter, which uses cytoplasmic anions as extrinsic voltage sensors and can operate at microsecond rates. As prestin mediates changes in outer hair cell length in response to membrane potential variations, it might be responsible for sound amplification in the mammalian hearing organ.

3. Waldegger S, Moschen I, Ramirez A, Smith R J, Ayadi F H, Lang F, Kubisch C. (2001) Cloning and characterization of SLC26A6, a novel member of the solute carrier 26 gene family. Genomics. 72(1):43-50.

The SLC26 gene family (solute carrier family 26) comprises five mammalian genes that encode anion transporter-related proteins. In addition to sat-1 and prestin, which were cloned from rat and gerbil, respectively, three human members have been identified and associated with specific genetic diseases (DTD, diastrophic dysplasia; CLD, congenital chloride diarrhea; PDS, Pendred syndrome). In this study we used a homology approach combined with RACE PCR to identify human SLC26A6, the sixth member of this gene family. Northern blot analysis showed the highest SLC26A6 transcript levels in kidney and pancreas. Expression in MDCK cells and in Xenopus oocytes demonstrated trafficking of the SLC26A6 protein to the cell membrane but did not reveal anion transport activity with tracer uptake or intracellular pH measurements. We determined the genomic structure of the SLC26A6 gene and excluded mutations in the 21 coding exons as the cause of DFNB6 and USH2B, which closely map to the SLC26A6 chromosomal locus (3p21).

PMID: 11247665

Panel 4D Summary: Ag3315 Expression of the CG57721-01 gene is limited to small airway epithelium (CT=35) and normal lung (CT-31.7) on this panel. Interestingly, expression of this gene in small airway epithelium is strongly upregulated by treatment with TNF-alpha and IL-1 beta (CT=30.4). This observation suggests that expression of this gene could be used as a marker for activated epithelium. Furthermore, therapeutic modulation of the activity of this gene or its protein product using small molecule drugs could be of use in the treatment of asthma and emphysema.

NOV50

Expression of NOV50/CG57787-01 was assessed using the primer-probe sets Ag3332, Ag2005 and Ag2259, described in Tables AQA, AQB and AQC. Results of the RTQ-PCR runs are shown in Tables AQD, AQE, AQF and AQG.

TABLE AQA
Probe Name Ag3332

| Primers | Sequences | Length | Start Position | SEQ ID NO |
|---|---|---|---|---|
| Forward | 5′-atttcctaccccgtcattaaag-3′ | 22 | 67 | 407 |
| Probe | TET-5′-caccatcggctttggacagatcaag-3′-TAMRA | 25 | 111 | 408 |
| Reverse | 5′-gttctgtagtcccagcaggtt-3′ | 21 | 136 | 409 |

TABLE AQB
Probe Name Ag2005

| Primers | Sequences | Length | Start Position | SEQ ID NO |
|---|---|---|---|---|
| Forward | 5′-gttcctgctggacttcatttc-3′ | 21 | 51 | 410 |
| Probe | TET-5′-accccgtcattaaaggcttcacctct-3′-TAMRA | 26 | 74 | 411 |
| Reverse | 5′-caggttcttgatctgtccaaag-3′ | 22 | 120 | 412 |

TABLE AQC
Probe Name Ag2259

| Primers | Sequences | Length | Start Position | SEQ ID NO |
|---|---|---|---|---|
| Forward | 5′-gttcctgctggacttcatttc-3′ | 21 | 51 | 413 |
| Probe | TET-5′-accccgtcattaaaggcttcacctct-3′-TAMRA | 26 | 74 | 414 |
| Reverse | 5′-caggttcttgatctgtccaaag-3′ | 22 | 120 | 415 |

TABLE AQD
CNS_neurodegeneration_v1.0

| | Rel. Exp. (%) Ag3332, Run | | Rel. Exp. (%) Ag3332, Run |
|---|---|---|---|
| Tissue Name | 210146364 | Tissue Name | 210146364 |
| AD 1 Hippo | 16.4 | Control (Path) 3 Temporal | 3.9 |
| | | Ctx | |
| AD 2 Hippo | 56.6 | Control (Path) 4 Temporal | 44.4 |
| | | Ctx | |
| AD 3 Hippo | 7.1 | AD 1 Occipital Ctx | 15.6 |
| AD 4 Hippo | 13.2 | AD 2 Occipital Ctx | 0.0 |
| | | (Missing) | |
| AD 5 hippo | 97.3 | AD 3 Occipital Ctx | 5.7 |
| AD 6 Hippo | 59.0 | AD 4 Occipital Ctx | 28.9 |
| Control 2 Hippo | 40.9 | AD 5 Occipital Ctx | 27.5 |
| Control 4 Hippo | 23.7 | AD 6 Occipital Ctx | 88.3 |
| Control (Path) 3 Hippo | 8.7 | Control 1 Occipital Ctx | 2.6 |
| AD 1 Temporal Ctx | 18.7 | Control 2 Occipital Ctx | 53.6 |
| AD 2 Temporal Ctx | 45.1 | Control 3 Occipital Ctx | 19.1 |
| AD 3 Temporal Ctx | 9.9 | Control 4 Occipital Ctx | 9.9 |
| AD 4 Temporal Ctx | 40.9 | Control (Path) 1 Occipital | 81.8 |
| | | Ctx | |
| AD 5 Inf Temporal Ctx | 100.0 | Control (Path) 2 Occipital | 9.5 |
| | | Ctx | |
| AD 5 SupTemporal Ctx | 87.1 | Control (Path) 3 Occipital | 3.4 |
| | | Ctx | |
| AD 6 Inf Temporal Ctx | 62.4 | Control (Path) 4 Occipital | 16.5 |
| | | Ctx | |
| AD 6 Sup Temporal Ctx | 62.0 | Control 1 Parietal Ctx | 6.6 |
| Control 1 Temporal Ctx | 5.3 | Control 2 Parietal Ctx | 83.5 |
| Control 2 Temporal Ctx | 46.7 | Control 3 Parietal Ctx | 17.8 |
| Control 3 Temporal Ctx | 31.6 | Control (Path) 1 Parietal | 99.3 |
| | | Ctx | |
| Control 4 Temporal Ctx | 14.7 | Control (Path) 2 Parietal | 19.9 |
| | | Ctx | |
| Control (Path) 1 Temporal | 84.1 | Control (Path) 3 Parietal | 3.7 |
| Ctx | | Ctx | |
| Control (Path) 2 Temporal | 41.8 | Control (Path) 4 Parietal | 47.3 |
| Ctx | | Ctx | |

TABLE AQE
General_screening_panel_v1.4

| Tissue Name | 215678734 | Tissue Name | 215678734 |
|---|---|---|---|
| Adipose | 6.6 | Renal ca. TK-10 | 100.0 |
| Melanoma* Hs688(A).T | 28.5 | Bladder | 18.0 |
| Melanoma* Hs688(B).T | 31.0 | Gastric ca. (liver met.) NCI- | 17.1 |
| | | N87 | |
| Melanoma* M14 | 30.4 | Gastric ca. KATO III | 19.3 |
| Melanoma* LOXIMVI | 22.5 | Colon ca. SW-948 | 9.0 |
| Melanoma* SK-MEL-5 | 30.1 | Colon ca. SW480 | 0.7 |
| Squamous cell carcinoma | 0.2 | Colon ca.* (SW480 met) SW620 | 1.9 |
| SCC-4 | | | |
| Testis Pool | 5.3 | Colon ca. HT29 | 4.9 |
| Prostate ca.* (bone met) | 53.6 | Colon ca. HCT-116 | 28.9 |
| PC-3 | | | |
| Prostate Pool | 12.7 | Colon ca. CaCo-2 | 49.0 |
| Placenta | 55.9 | Colon cancer tissue | 12.9 |
| Uterus Pool | 3.7 | Colon ca. SW1116 | 6.8 |
| Ovarian ca. OVCAR-3 | 42.0 | Colon ca. Colo-205 | 0.0 |
| Ovarian ca. SK-OV-3 | 40.6 | Colon ca. SW-48 | 16.7 |
| Ovarian ca. OVCAR-4 | 19.9 | Colon Pool | 26.6 |
| Ovarian ca. OVCAR-5 | 57.0 | Small Intestine Pool | 46.0 |
| Ovarian ca. IGROV-1 | 30.4 | Stomach Pool | 13.7 |
| Ovarian ca. OVCAR-8 | 9.7 | Bone Marrow Pool | 5.9 |
| Ovary | 18.3 | Fetal Heart | 14.0 |
| Breast ca. MCF-7 | 35.1 | Heart Pool | 10.0 |
| Breast ca. MDA-MB-231 | 19.5 | Lymph Node Pool | 16.8 |
| Breast ca. BT 549 | 37.4 | Fetal Skeletal Muscle | 4.2 |
| Breast ca. T47D | 97.3 | Skeletal Muscle Pool | 6.9 |
| Breast ca. MDA-N | 13.6 | Spleen Pool | 17.7 |
| Breast Pool | 22.7 | Thymus Pool | 14.8 |
| Trachea | 21.0 | CNS cancer (glio/astro) U87- | 9.0 |
| | | MG | |
| Lung | 4.0 | CNS cancer (glio/astro) U- | 24.7 |
| | | 118-MG | |
| Fetal Lung | 53.2 | CNS cancer (neuro; met) SK- | 79.6 |
| | | N-AS | |
| Lung ca. NCI-N417 | 0.9 | CNS cancer (astro) SF-539 | 3.7 |
| Lung ca. LX-1 | 0.8 | CNS cancer (astro) SNB-75 | 54.7 |
| Lung ca. NCI-H146 | 12.4 | CNS cancer (glio) SNB-19 | 24.0 |
| Lung ca. SHP-77 | 24.3 | CNS cancer (glio) SF-295 | 58.6 |
| Lung ca. A549 | 33.9 | Brain (Amygdala) Pool | 13.9 |
| Lung ca. NCI-H526 | 20.0 | Brain (cerebellum) | 14.9 |
| Lung ca. NCI-H23 | 30.4 | Brain (fetal) | 40.3 |
| Lung ca. NCI-H460 | 34.9 | Brain (Hippocampus) Pool | 25.5 |
| Lung ca. HOP-62 | 4.0 | Cerebral Cortex Pool | 23.8 |
| Lung ca. NCI-H522 | 72.2 | Brain (Substantia nigra) Pool | 35.4 |
| Liver | 5.0 | Brain (Thalamus) Pool | 29.9 |
| Fetal Liver | 17.4 | Brain (whole) | 10.6 |
| Liver ca. HepG2 | 26.4 | Spinal Cord Pool | 24.8 |
| Kidney Pool | 35.6 | Adrenal Gland | 65.1 |
| Fetal Kidney | 14.6 | Pituitary gland Pool | 17.0 |
| Renal ca 786-0 | 0.9 | Salivary Gland | 11.6 |
| Renal ca. A498 | 16.4 | Thyroid (female) | 17.9 |
| Renal ca. ACHN | 11.4 | Pancreatic ca. CAPAN2 | 2.4 |
| Renal ca. UO-31 | 4.6 | Pancreas Pool | 21.0 |

TABLE AQF
Panel 1.3D

| | Rd. Exp. (%) Ag2005, Run | | Rel. Exp.(%) Ag2005, Run |
|---|---|---|---|
| Tissue Name | 165981814 | Tissue Name | 165981814 |
| Liver adenocarcinoma | 36.6 | Kidney (Fetal) | 33.2 |
| Pancreas | 14.4 | Renal ca. 786-0 | 0.0 |
| Pancreatic ca. CAPAN 2 | 0.9 | Renal ca. A498 | 12.8 |
| Adrenal gland | 46.3 | Renal ca. RXF 393 | 20.9 |
| Thyroid | 9.6 | Renal ca. ACHN | 3.3 |
| Salivary gland | 20.7 | Renal ca. UO-31 | 5.4 |
| Pituitary gland | 58.2 | Renal ca. TK-10 | 40.1 |
| Brain (fetal) | 28.7 | Liver | 3.1 |
| Brain (whole) | 92.7 | Liver (fetal) | 10.7 |
| Brain (amygdala) | 78.5 | Liver ca. (hepatoblast) | 15.6 |
| | | HepG2 | |
| Brain (cerebellum) | 12.7 | Lung | 27.9 |
| Brain (hippocampus) | 53.2 | Lung (fetal) | 53.2 |
| Brain (substantia nigra) | 52.1 | Lung ca. (small cell) LX-1 | 0.0 |
| Brain (thalamus) | 90.8 | Lung ca. (small cell) NCl- | 6.6 |
| | | H69 | |
| Cerebral Cortex | 100.0 | Lung ca. (s.cell var.) SHP- | 22.4 |
| | | 77 | |
| Spinal cord | 56.6 | Lung ca. (large cell)NCl- | 15.9 |
| | | H460 | |
| glio/astro U87-MG | 6.0 | Lung ca. (non-sm. cell) | 9.8 |
| | | A549 | |
| glio/astro U-118 MG | 7.6 | Lung ca. (non-s.cell) NCl- | 13.7 |
| | | H23 | |
| astrocytoma SW1783 | 16.4 | Lung ca (non-s cell) HOP- | 8.4 |
| | | 62 | |
| neuro*; met SK-N-AS | 26.8 | Lung ca. (non-s.cl) NCl- | 15.8 |
| | | H522 | |
| astrocytoma SF-539 | 3.1 | Lung ca. (squam.) SW 900 | 28.1 |
| | | H596 | |
| astrocytoma SNB-75 | 12.9 | Lung ca. (squam.) NCl-2 | 2.6 |
| | | H596 | |
| glioma SNB-19 | 13.1 | Mammary gland | 10.5 |
| glioma U251 | 8.2 | Breast ca.* (pl.ef) MCF-7 | 14.8 |
| glioma SF-295 | 19.3 | Breast ca * (pl.ef) MDA- | 10.4 |
| | | MB-231 | |
| Heart (fetal) | 28.5 | Breast ca.* (pl.ef) T47D | 28.7 |
| Heart | 4.8 | Breast ca. BT-549 | 5.3 |
| Skeletal muscle (fetal) | 16.5 | Breast ca. MDA-N | 3.6 |
| Skeletal muscle | 13.9 | Ovary | 39.2 |
| Bone marrow | 2.6 | Ovarian ca. OVCAR-3 | 11.4 |
| Thymus | 16.4 | Ovarian ca. OVCAR-4 | 51.4 |
| Spleen | 23.5 | Ovarian ca. OVCAR-5 | 20.4 |
| Lymph node | 37.4 | Ovarian ca. OVCAR-8 | 12.2 |
| Colorectal | 12.4 | Ovarian ca. IGROV-1 | 12.2 |
| Stomach | 14.6 | Ovarian ca. ascites) SK- | 15.5 |
| | | OV-3 | |
| Small intestine | 18.6 | Uterus | 20.3 |
| Colon ca. SW480 | 0.0 | Placenta | 71.2 |
| Colon ca.* SW620(SW480 | 1.5 | Prostate | 14.1 |
| met) | | | |
| Colon ca. HT29 | 0.7 | Prostate ca.* (bone met)PC- | 22.7 |
| | | 3 | |
| Colon ca. HCT-116 | 6.8 | Testis | 8.6 |
| Colon ca. CaCo-2 | 13.7 | Melanoma Hs688(A).T | 11.7 |
| Colon ca. tissue(ODO3866) | 13.5 | Melanoma* (met) | 8.8 |
| | | Hs688(B).T | |
| Colon ca. HCC-2998 | 18.2 | Melanoma UACC-62 | 28.7 |
| Gastric ca.* (liver met) NCl- | 9.7 | Melanoma M14 | 17.4 |
| N87 | | | |
| Bladder | 6.8 | Melanoma LOX IMVI | 2.6 |
| Trachea | 6.3 | Melanoma* (met) SK- | 16.6 |
| | | MEL-5 | |
| Kidney | 28.7 | Adipose | 5.6 |

TABLE AQG
Panel 4D

| | Rel. Exp. (%) | Rel. Exp. (%) | | Rel. Exp. (%) | Rel. Exp. (%) |
|---|---|---|---|---|---|
| | Ag2259, Run | Ag3332, Run | | Ag2259, Run | Ag3332, Run |
| Tissue Name | 150910665 | 165725931 | Tissue Name | 150910665 | 165725931 |
| Secondary Th1 act | 3.1 | 2.7 | HUVEC IL-1beta | 1.9 | 1.2 |
| Secondary Th2 act | 2.4 | 1.2 | HUVEC IFN gamma | 8.5 | 3.5 |
| Secondary Tr1 act | 6.6 | 6.7 | HUVEC TNF alpha + | 3.5 | 1.5 |
| | | | IFN gamma | | |
| Secondary Th1 rest | 1.8 | 2.8 | HUVEC TNF alpha + | 3.8 | 1.4 |
| | | | IL4 | | |
| Secondary Th2 rest | 4.5 | 4.1 | HUVEC IL-11 | 2.2 | 1.7 |
| Secondary Tr1 rest | 2.5 | 2.9 | Lung Microvascular EC | 6.8 | 2.5 |
| | | | none | | |
| Primary Th1 act | 1.0 | 0.4 | Lung Microvascular EC | 4.7 | 2.1 |
| | | | TNF alpha + IL-1beta | | |
| Primary Th2 act | 3.2 | 2.7 | Microvascular Dermal | 3.5 | 2.5 |
| | | | EC none | | |
| Primary Tr1 act | 1.2 | 0.9 | Microsvasular Dermal | 3.0 | 2.1 |
| | | | EC TNF alpha + IL- | | |
| | | | 1beta | | |
| Primary Th1 rest | 2.0 | 2.0 | Bronchial epithelium | 1.7 | 1.1 |
| | | | TNF alpha + IL1beta | | |
| Primary Th2 rest | 3.3 | 2.4 | Small airway epithelium | 1.4 | 0.3 |
| | | | none | | |
| Primary Tr1 rest | 2.0 | 1.3 | Small airway epithelium | 1.6 | 0.8 |
| | | | TNF alpha + IL-1beta | | |
| CD45RA CD4 | 2.0 | 1.3 | Coronery artery SMC | 2.3 | 1.4 |
| lymphocyte act | | | rest | | |
| CD45RO CD4 | 3.3 | 2.4 | Coronery artery SMC | 2.3 | 1.1 |
| lymphocyte act | | | TNF alpha + IL-1beta | | |
| CD8 lymphocyte act | 2.6 | 1.5 | Astrocytes rest | 0.4 | 0.8 |
| Secondary CD8 | 6.1 | 6.1 | Astrocytes TNF alpha + | 1.6 | 2.6 |
| lymphocyte rest | | | IL-1beta | | |
| Secondary CD8 | 0.3 | 0.6 | KU-812 (Basophil) rest | 0.0 | 0.0 |
| lymphocyte act | | | | | |
| CD4 lymphocyte none | 5.6 | 5.3 | KU-812 (Basophil) | 0.2 | 0.0 |
| | | | PMA/ionomycin | | |
| 2ry Th1/Th2/Tr1_anti- | 1.9 | 2.5 | CCD1106 | 0.7 | 0.4 |
| CD95 CH11 | | | (Keratinocytes) none | | |
| LAK cells rest | 25.5 | 17.2 | CCD1106 | 1.1 | 3.4 |
| | | | (Keratinocytes) | | |
| | | | TNF alpha + IL-1beta | | |
| LAK cells IL-2 | 3.8 | 3.9 | Liver cirrhosis | 1.2 | 1.9 |
| LAK cells IL-2 + IL-12 | 15.0 | 8.8 | Lupus kidney | 1.1 | 3.3 |
| LAK cells IL-2 + IFN | 16.2 | 13.8 | NCI-H292 none | 4.5 | 2.2 |
| gamma | | | | | |
| LAK cells IL-2 + IL- | 12.6 | 7.9 | NCI-H292 IL-4 | 8.0 | 3.7 |
| 18 | | | | | |
| LAK cells | 15.7 | 12.7 | NCI-H292 IL-9 | 6.4 | 4.5 |
| PMA/ionomycin | | | | | |
| NK Cells IL-2 rest | 5.4 | 2.9 | NCI-H292 IL-13 | 4.5 | 2.6 |
| Two Way MLR 3 day | 14.7 | 9.9 | NCI-H292 IFN gamma | 3.3 | 1.4 |
| Two Way MLR 5 day | 9.6 | 7.3 | HPAEC none | 6.4 | 3.7 |
| Two Way MLR 7 day | 2.5 | 1.5 | HPAEC TNF alpha + | 8.8 | 5.3 |
| | | | IL-1beta | | |
| PBMC rest | 3.5 | 4.3 | Lung fibroblast none | 6.7 | 10.1 |
| PBMC PWM | 14.3 | 4.1 | Lung fibroblast TNF | 3.7 | 9.4 |
| | | | alpha + IL-1beta | | |
| PBMC PHA-L | 2.4 | 0.7 | Lung fibroblast IL-4 | 13.3 | 5.6 |
| Ramos (B cell) none | 0.0 | 0.1 | Lung fibroblast IL-9 | 8.5 | 4.7 |
| Ramos (B cell) | 0.2 | 0.0 | Lung fibroblast IL-13 | 8.7 | 4.2 |
| ionomycin | | | | | |
| B lymphocytes PWM | 3.7 | 0.7 | Lung fibroblast IFN | 9.0 | 5.1 |
| | | | gamma | | |
| B lymphocytes | 10.2 | 3.0 | Dermal fibroblast | 4.6 | 2.3 |
| CD40L and IL-4 | | | CCD1070 rest | | |
| EOL-1 dbcAMP | 4.2 | 2.9 | Dermal fibroblast | 4.2 | 3.1 |
| | | | CCD1070 TNF alpha | | |
| EOL-1 dbcAMP | 4.3 | 4.5 | Dermal fibroblast | 3.7 | 1.3 |
| PMA/ionomycin | | | CCD1070 IL-1beta | | |
| Dendritic cells none | 77.4 | 74.7 | Dermal fibroblast IFN | 3.5 | 1.8 |
| | | | gamma | | |
| Dendritic cells LPS | 30.4 | 19.3 | Dermal fibroblast IL-4 | 6.7 | 4.1 |
| Dendritic cells anti- | 95.3 | 100.0 | IBD Colitis 2 | 0.6 | 0.5 |
| CD40 | | | | | |
| Monocytes rest | 7.0 | 3.3 | IBD Crohn's | 0.5 | 0.6 |
| Monocytes LPS | 18.8 | 18.4 | Colon | 4.2 | 11.0 |
| Macrophages rest | 100.0 | 89.5 | Lung | 4.5 | 3.1 |
| Macrophages LPS | 15.2 | 10.2 | Thymus | 13.8 | 6.1 |
| HUVEC none | 3.5 | 2.4 | Kidney | 3.9 | 1.9 |
| HUVEC starved | 7.7 | 6.8 | | | |

CNS_neurodegeneration_v1.0 Summary: Ag3332 This panel confirms the expression of the CG57787-01 gene at moderate levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3332 Expression of the CG57787-01 gene is highest in renal cell carcinoma cell line TK-10 (CT=28.3). In addition, levels of expression of this gene are higher in fetal lung (CT=29) and 7 lung cancer cell lines than in the adult lung (CT=33). Thus, expression of this gene could be used to differentiate adult and fetal lung and also as marker to detect the presence of lung cancer. Furthermore, therapeutic modulation of the expression or function of this gene may be effective in the treatment of lung cancer.

In addition, this gene is expressed at moderate levels throughout the central nervous system, including in amygdala, cerebellum, hippocampus, cerebral cortex, substantia nigra, thalamus and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at moderate levels in pancreas, adrenal gland, pituitary gland, adipose, thyroid, skeletal muscle, heart, and liver. Therefore, therapeutic modulation of the activity of this gene may prove useful in the treatment of endocrine/metabolically related diseases, such as obesity and diabetes.

Panel 1.3D Summary: Ag3332 Expression of the CG57787-01 gene is highest in cerebral cortex (CT=31.2). In addition, this gene is expressed at moderate levels throughout the central nervous system, including in amygdala, cerebellum, hippocampus, cerebral cortex, substantia nigra, thalamus and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at moderate levels in pancreas, adrenal gland, pituitary gland, adipose, thyroid, skeletal muscle, heart, and liver. Therefore, therapeutic modulation of the activity of this gene may prove useful in the treatment of endocrine/metabolically related diseases, such as obesity and diabetes.

Panel 4D Summary: Ag2259/Ag3332 Results from two experiments using different probe/primer sets are in excellent agreement. Expression of the CG57787-01 gene is highest in treated and untreated dendritic cells and macrophages. Dendritic cells and macrophages are powerful antigen-presenting cells (APC) whose function is pivotal in the initiation and maintenance of normal immune responses. Autoimmunity and inflammation may also be reduced by suppresssion of this function. Therefore, therapeutic utilization of the protein encoded by this transcript may be important in the treatment of diseases where antigen presentation, a function of mature dendritic cells, plays an important role, such as asthma, rheumatoid arthrtis, IBD, and psoriasis.

AR. CG57785-01: Sulfate Transporter

Expression of gene CG57785-01 was assessed using the primer-probe set Ag3331, described in Table ARA. Results of the RTQ-PCR runs are shown in Tables ARB, ARC and ARD.

TABLE ARA
Probe Name Ag3331

| Primers | Sequences | Length | Start Position | SEQ ID NO |
|---|---|---|---|---|
| Forward | 5′-ctcatatttctgggcaagaaga-3′ | 22 | 646 | 416 |
| Probe | TET-5′-tgccagtcttcacaattacagtgtca-3′-TAMRA | 26 | 669 | 417 |
| Reverse | 5′-cgatggctattaaatcctggtt-3′ | 22 | 700 | 418 |

TABLE ARB
CNS_neurodegeneration_v1.0

| | Rel. Exp. (%) Ag3331, Run | | Rel. Exp. (%) Ag3331, Run |
|---|---|---|---|
| Tissue Name | 210146363 | Tissue Name | 210146363 |
| AD 1 Hippo | 15.5 | Control (Path) 3 Temporal | 4.5 |
| | | Ctx | |
| AD 2 Hippo | 39.2 | Control (Path) 4 Temporal | 57.0 |
| | | Ctx | |
| AD 3 Hippo | 3.3 | AD 1 Occipital Ctx | 24.7 |
| AD 4 Hippo | 6.3 | AD 2 Occipital Ctx | 0.0 |
| | | (Missing) | |
| AD 5 Hippo | 84.7 | AD 3 Occipital Ctx | 0.0 |
| AD 6 Hippo | 40.9 | AD 4 Occipital Ctx | 18.4 |
| Control 2 Hippo | 35.6 | AD 5 Occipital Ctx | 82.4 |
| Control 4 Hippo | 7.3 | AD 6 Occipital Ctx | 30.8 |
| Control (Path) 3 Hippo | 2.0 | Control 1 Occipital Ctx | 3.1 |
| AD 1 Temporal Ctx | 36.9 | Control 2 Occipital Ctx | 38.2 |
| AD 2 Temporal Ctx | 62.4 | Control 3 Occipital Ctx | 33.4 |
| AD 3 Temporal Ctx | 16.0 | Control 4 Occipital Ctx | 2.8 |
| AD 4 Temporal Ctx | 26.4 | Control (Path) 1 Occipital | 93.3 |
| | | Ctx | |
| AD 5 Inf Temporal Ctx | 68.3 | Control (Path) 2 Occipital | 4.4 |
| | | Ctx | |
| AD 5 Sup Temporal Ctx | 44.1 | Control (Path) 3 Occipital | 5.1 |
| | | Ctx | |
| AD 6 Inf Temporal Ctx | 68.3 | Control (Path) 4 Occipital | 22.4 |
| | | Ctx | |
| AD 6 Sup Temporal Ctx | 64.6 | Control 1 Parietal Ctx | 7.6 |
| Control 1 Temporal Ctx | 6.2 | Control 2 Parietal Ctx | 48.3 |
| Control 2 Temporal Ctx | 62.9 | Control 3 Parietal Ctx | 41.5 |
| Control 3 Temporal Ctx | 45.4 | Control (Path) 1 Parietal | 84.7 |
| | | Ctx | |
| Control 3 Temporal Ctx | 7.1 | Control (Path) 2 Parietal | 31.2 |
| | | Ctx | |
| Control (Path) 1 Temporal | 100.0 | Control (Path) 3 Parietal | 1.0 |
| Ctx | | Ctx | |
| Control (Path) 2 Temporal | 57.8 | Control (Path) 4 Parietal | 67.8 |
| Ctx | | Ctx | |

TABLE ARC
General_screening_panel_v1.4

| | Rel. Exp. (%) Ag3331, Run | | Rel. Exp. (%) Ag3331, Run |
|---|---|---|---|
| Tissue Name | 215678710 | Tissue Name | 215678710 |
| Adipose | 0.4 | Renal ca. TK-10 | 0.0 |
| Melanoma* Hs688(A).T | 0.0 | Bladder | 0.3 |
| Melanoma* Hs688(B).T | 0.0 | Gastric ca. (liver met.) NCI- | 0.2 |
| | | N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.2 |
| Squamous cell carcinoma | 0.0 | Colon ca.* (SW480 met) | 0.0 |
| SCC-4 | | SW620 | |
| Testis Pool | 100.0 | Colon ca. HT29 | 0.0 |
| Prostate ca.* (bone met) | 0.0 | Colon ca. HCT-116 | 0.0 |
| PC-3 | | | |
| Prostate Pool | 0.1 | Colon ca. CaCo-2 | 0.0 |
| Placenta | 0.2 | Colon cancer tissue | 0.2 |
| Uterus Pool | 0.1 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 0.5 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.1 |
| Ovary | 0.0 | Fetal Heart | 0.0 |
| Breast ca. MCF-7 | 0.0 | Heart Pool | 0.1 |
| Breast ca. MDA-MB-231 | 0.0 | Lymph Node Pool | 0.3 |
| Breast ca. BT 549 | 0.0 | Fetal Skeletal Muscle | 0.2 |
| Breast ca. T47D | 0.2 | Skeletal Muscle Pool | 0.0 |
| Breast ca. MDA-N | 0.0 | Spleen Pool | 1.3 |
| Breast Pool | 0.0 | Thymus Pool | 0.6 |
| Trachea | 0.6 | CNS cancer (glio/astro) U87- | 0.0 |
| | | MG | |
| Lung | 0.0 | CNS cancer (glio/astro) U- | 0.0 |
| | | 118-MG | |
| Fetal Lung | 0.5 | CNS cancer (neuro; met) SK- | 0.0 |
| | | N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.6 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 4.0 |
| Lung ca. NCI-H526 | 1.2 | Brain (cerebellum) | 12.8 |
| Lung ca. NCI-H23 | 0.0 | Brain (fetal) | 12.4 |
| Lung ca. NCI-H460 | 0.0 | Brain (Hippocampus) Pool | 3.9 |
| Lung ca. HOP-62 | 0.0 | Cerebral Cortex Pool | 5.0 |
| Lung ca. NCI-H522 | 0.1 | Brain (Substantia nigra) Pool | 4.2 |
| Liver | 0.0 | Brain (Thalamus) Pool | 6.9 |
| Fetal Liver | 0.1 | Brain (whole) | 6.7 |
| Liver ca. HepG2 | 0.0 | Spinal Cord Pool | 0.2 |
| Kidney Pool | 0.1 | Adrenal Gland | 0.1 |
| Fetal Kidney | 0.3 | Pituitary gland Pool | 0.0 |
| Renal ca. 786-0 | 0.0 | Salivary Gland | 0.0 |
| Renal ca. A498 | 0.0 | Thyroid (female) | 0.5 |
| Renal ca. ACHN | 0.0 | Pancreatic ca. CAPAN2 | 0.0 |
| Renal ca. UO-31 | 0.0 | Pancreas Pool | 0.1 |

TABLE ARD
Panel 4D

| | Rel. Exp. (%) Ag3331, Run | | Rel. Exp. (%) Ag3331, Run |
|---|---|---|---|
| Tissue Name | 165084166 | Tissue Name | 165084166 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + | 0.0 |
| | | IL-1beta | |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 0.0 | Bronchial epithelium TNF alpha + | 0.0 |
| | | IL1beta | |
| Primary Th2 rest | 0.0 | Small airway epithelium none | 0.0 |
| Primary Tr1 rest | 0.0 | Small airway epithelium TNF alpha + | 9.3 |
| | | IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNF alpha + | 0.0 |
| act | | IL-1beta | |
| CD8 lymphocyte act | 0.0 | Astrocytes rest | 0.0 |
| Secondary CD8 lymphocyte | 0.0 | Astrocytes TNF alpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 9.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 6.7 | Lung fibroblast TNF alpha + IL- | 10.5 |
| | | 1beta | |
| PBMC PHA-L | 9.3 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 rest | 0.0 |
| IL-4 | | | |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL- | 0.0 |
| PMA/ionomycin | | 1beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monycytes rest | 100.0 | IBD Crohn's | 10.4 |
| Monocytes LPS | 9.2 | Colon | 7.6 |
| Macrophages rest | 6.7 | Lung | 0.0 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag3331 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_sereening_panel_v1.4 Summary: Ag3331 Expression of the CG57785-01 gene is highest in a sample derived from testis (CT=28.6). Thus, the expression of this gene could be used to distinguish testis from the other samples in the panel. In addition, therapeutic modulation of the activity of this gene or its protein product, through the use of small molecule drugs, protein therapeutics or antibodies, might be beneficial in the treatment of fertility and hypogonadism.

This gene is also expressed at low to moderate levels throughout the CNS, including in amygdala, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4D Summary: The CG57785-01 transcript is expressed at detectable levels only in resting monocytes (CT=34.3). Thus, the expression of this gene could be used to distinguish resting monocytes from the other samples in the panel. In addition, the protein encoded by this transcript may be important in monocytic differentiation and activation. Therefore, regulating the expression of this transcript or the function of the protein it encodes may alter the types and levels of monocytic cells regulated by cytokine and chemokine production and T cell activation. Therapeutics designed with the protein encoded by this transcript could therefore be important for the treatment of asthma, emphysema, inflammatory bowel disease, arthritis and psoriasis.

AS. CG57748-01: N-acetylgalactosaminyltransferase

Expression of gene CG57748-01 was assessed using the primer-probe set Ag3325, described in Table ASA.

TABLE ASA
Probe Name Ag3325

| Primers | Sequences | Length | Start Position | SEQ ID NO |
|---|---|---|---|---|
| Forward | 5′-tggcaccttctaatgttcaaat-3′ | 22 | 1942 | 419 |
| Probe | TET-5′-tttccaggtactcagatggtacctg-3′-TAMRA | 26 | 1969 | 420 |
| Reverse | 5′-cgcgtgttaaatacgtttgaag-3′ | 22 | 2018 | 421 |

CNS_neurodegeneration_v1.0 Summary: Ag3325 Expression of this gene is low/undetectable (CTs>35) across all of the samples on this panel (data not shown).

General_screening_panel_v1.4 Summary: Ag3325 Expression of this gene is low/undetectable (CTs>35) across all of the samples on this panel (data not shown).

Panel 4D Summary: Ag3325 Expression of this gene is low/undetectable (CTs>35) across all of the samples on this panel due to a probable experimental failure (data not shown).

AT. CG57693-01: Novel Protein Kinase

Expression of gene CG57693-01 was assessed using the primer-probe set Ag3309, described in Table ATA. Results of the RTQ-PCR runs are shown in Tables ATB, ATC and ATD.

TABLE ATA
Probe Name Ag3309

| Primers | Sequences | Length | Start Position | SEQ ID NO |
|---|---|---|---|---|
| Forward | 5′-ttctacatggctcctgaagtct-3′ | 22 | 1540 | 422 |
| Probe | TET-5′-actacacagccaaggcggacatcttt-3′-TAMRA | 26 | 1571 | 423 |
| Reverse | 5′-tctttctatcattgcccagatg-3′ | 22 | 1611 | 424 |

TABLE ATB
CNS_neurodegeneration_v1.0

| | Rel. Exp. (%) Ag3309, Run | | Rel. Exp. (%) Ag3309, Run |
|---|---|---|---|
| Tissue Name | 210143041 | Tissue Name | 210143041 |
| AD 1 Hippo | 9.5 | Control (Path) 3 Temporal | 3.7 |
| | | Ctx | |
| AD 2 Hippo | 21.2 | Control (Path) 4 Temporal | 26.2 |
| | | Ctx | |
| AD 3 Hippo | 4.6 | AD 1 Occipital Ctx | 19.5 |
| AD 4 Hippo | 3.0 | AD 2 Occipital Ctx | 0.0 |
| | | (Missing) | |
| AD 5 hippo | 100.0 | AD 3 Occipital Ctx | 5.4 |
| AD 6 Hippo | 44.4 | AD 4 Occipital Ctx | 12.2 |
| Control 2 Hippo | 18.9 | AD 5 Occipital Ctx | 16.4 |
| Control 4 Hippo | 7.7 | AD 6 Occipital Ctx | 40.6 |
| Control (Path) 3 Hippo | 3.9 | Control 1 Occipital Ctx | 3.5 |
| AD 1 Temporal Ctx | 13.1 | Control 2 Occipital Ctx | 63.3 |
| AD 2 Temporal Ctx | 21.9 | Control 3 Occipital Ctx | 17.2 |
| AD 3 Temporal Ctx | 6.4 | Control 4 Occipital Ctx | 4.2 |
| AD 4 Temporal Ctx | 17.6 | Control (Path) 1 Occipital | 72.2 |
| | | Ctx | |
| AD 5 Inf Temporal Ctx | 83.5 | Control (Path) 2 Occipital | 8.1 |
| | | Ctx | |
| AD 5 SupTemporal Ctx | 31.0 | Control (Path) 3 Occipital | 2.7 |
| | | Ctx | |
| AD 6 Inf Temporal Ctx | 46.7 | Control (Path) 4 Occipital | 12.9 |
| | | Ctx | |
| AD 6 Sup Temporal Ctx | 43.8 | Control 1 Parietal Ctx | 5.4 |
| Control 1 Temporal Ctx | 6.3 | Control 2 Parietal Ctx | 39.2 |
| Control 2 Temporal Ctx | 32.5 | Control 3 Parietal Ctx | 11.0 |
| Control 3 Temporal Ctx | 13.3 | Control (Path) 1 Parietal | 54.7 |
| | | Ctx | |
| Control 4 Temporal Ctx | 5.0 | Control (Path) 2 Parietal | 15.5 |
| | | Ctx | |
| Control (Path) 1 Temporal | 46.0 | Control (Path) 3 Parietal | 2.6 |
| Ctx | | Ctx | |
| Control (Path) 2 Temporal | 28.5 | Control (Path) 4 Parietal | 37.1 |
| Ctx | | Ctx | |

TABLE ATC
General_screening_panel_v1.4

| | Rel. Exp. (%) Ag3309, Run | | Rel. Exp. (%) Ag3309, Run |
|---|---|---|---|
| Tissue Name | 215648362 | Tissue Name | 215648362 |
| Adipose | 8.1 | Renal ca. TK-10 | 45.4 |
| Melanoma* Hs688(A).T | 19.6 | Bladder | 13.9 |
| Melanoma* Hs688(B).T | 20.9 | Gastric ca. (liver met.) NCI- | 73.2 |
| | | N87 | |
| Melanoma* M14 | 35.6 | Gastric ca. KATO III | 100.0 |
| Melanoma* LOXIMVI | 28.9 | Colon ca. SW-948 | 8.4 |
| Melanoma* SK-MEL-5 | 31.4 | Colon ca. SW480 | 63.7 |
| Squamous cell carcinoma | 28.9 | Colon ca.* (SW480 met) | 52.9 |
| SCC-4 | | SW620 | |
| Testis Pool | 19.8 | Colon ca. HT29 | 26.4 |
| Prostate ca.* (bone met) | 72.7 | Colon ca. HCT-116 | 61.6 |
| PC-3 | | | |
| Prostate Pool | 4.9 | Colon ca. CaCo-2 | 47.3 |
| Placenta | 18.0 | Colon cancer tissue | 23.5 |
| Uterus Pool | 2.1 | Colon ca. SW1116 | 8.8 |
| Ovarian ca. OVCAR-3 | 24.1 | Colon ca. Colo-205 | 18.6 |
| Ovarian ca. SK-OV-3 | 40.6 | Colon ca. SW-48 | 19.6 |
| Ovarian ca. OVCAR-4 | 17.7 | Colon Pool | 10.2 |
| Ovarian ca. OVCAR-5 | 43.5 | Small Intestine Pool | 11.8 |
| Ovarian ca. IGROV-1 | 18.3 | Stomach Pool | 9.0 |
| Ovarian ca. OVCAR-8 | 13.7 | Bone Marrow Pool | 5.9 |
| Ovary | 6.5 | Fetal Heart | 7.1 |
| Breast ca. MCF-7 | 31.2 | Heart Pool | 4.7 |
| Breast ca. MDA-MB-231 | 37.6 | Lymph Node Pool | 12.2 |
| Breast ca. BT 549 | 36.6 | Fetal Skeletal Muscle | 3.8 |
| Breast ca. T47D | 68.3 | Skeletal Muscle Pool | 6.6 |
| Breast ca. MDA-N | 14.5 | Spleen Pool | 9.2 |
| Breast Pool | 9.6 | Thymus Pool | 13.6 |
| Trachea | 14.0 | CNS cancer (glio/astro) U87- | 16.7 |
| | | MG | |
| Lung | 2.3 | CNS cancer (glio/astro) U- | 62.9 |
| | | 118-MG | |
| Fetal Lung | 21.8 | CNS cancer (neuro; met) SK- | 30.1 |
| | | N-AS | |
| Lung ca. NCI-N417 | 4.9 | CNS cancer (astro) SF-539 | 12.9 |
| Lung ca. LX-1 | 52.5 | CNS cancer (astro) SNB-75 | 50.3 |
| Lung ca. NCI-H146 | 22.5 | CNS cancer (glio) SNB-19 | 16.4 |
| Lung ca. SHP-77 | 39.2 | CNS cancer (glio) SF-295 | 23.2 |
| Lung ca. A549 | 38.7 | Brain (Amygdala) Pool | 9.1 |
| Lung ca. NCI-H526 | 17.1 | Brain (cerebellum) | 23.8 |
| Lung ca. NCI-H23 | 39.2 | Brain (fetal) | 18.7 |
| Lung ca. NCI-H460 | 24.5 | Brain (Hippocampus) Pool | 9.2 |
| Lung ca. HOP-62 | 27.4 | Cerebral Cortex Pool | 11.7 |
| Lung ca. NCI-H522 | 37.9 | Brain (Substantia nigra) Pool | 7.9 |
| Liver | 2.4 | Brain (Thalamus) Pool | 14.0 |
| Fetal Liver | 25.3 | Brain (whole) | 16.8 |
| Liver ca. HepG2 | 49.7 | Spinal Cord Pool | 4.9 |
| Kidney Pool | 21.0 | Adrenal Gland | 24.1 |
| Fetal Kidney | 9.5 | Pituitary gland Pool | 3.7 |
| Renal ca. 786-0 | 16.4 | Salivary Gland | 8.8 |
| Renal ca. A498 | 6.0 | Thyroid (female) | 5.7 |
| Renal ca. ACHN | 16.7 | Pancreatic ca. CAPAN2 | 37.4 |
| Renal ca. UO-31 | 25.2 | Pancreas Pool | 14.8 |

TABLE ATD
Panel 4D

| | Rel. Exp. (%) Ag3309, Run | | Rel. Exp. (%) Ag3309, Run |
|---|---|---|---|
| Tissue Name | 164335487 | Tissue Name | 164335487 |
| Secondary Th1 act | 24.7 | HUVEC IL-1beta | 10.7 |
| Secondary Th2 act | 33.7 | HUVEC IFN gamma | 18.7 |
| Secondary Tr1 act | 31.9 | HUVEC TNF alpha + IFN gamma | 13.4 |
| Secondary Th1 rest | 5.8 | HUVEC TNF alpha + IL4 | 16.3 |
| Secondary Th2 rest | 7.9 | HUVEC IL-11 | 11.4 |
| Secondary Tr1 rest | 9.4 | Lung Microvascular EC none | 13.9 |
| Primary Th1 act | 28.3 | Lung Microvascular EC TNF alpha + | 15.7 |
| | | IL-1beta | |
| Primary Th2 act | 28.1 | Microvascular Dermal EC none | 19.3 |
| Primary Tr1 act | 37.9 | Microsvasular Dermal EC | 16.3 |
| | | TNF alpha + IL-1beta | |
| Primary Th1 rest | 49.0 | Bronchial epithelium TNF alpha + | 36.3 |
| | | IL1beta | |
| Primary Th2 rest | 26.8 | Small airway epithelium none | 9.5 |
| Primary Tr1 rest | 22.8 | Small airway epithelium TNF alpha + | 64.2 |
| | | IL-1beta | |
| CD45RA CD4 lymphocyte | 14.9 | Coronery artery SMC rest | 14.9 |
| act | | | |
| CD45RO CD4 lymphocyte | 23.7 | Coronery artery SMC TNF alpha + | 8.1 |
| act | | IL-1beta | |
| CD8 lymphocyte act | 17.3 | Astrocytes rest | 14.6 |
| Secondary CD8 lymphocyte | 21.8 | Astrocytes TNF alpha + IL-1beta | 9.0 |
| rest | | | |
| Secondary CD8 lymphocyte | 15.7 | KU-812 (Basophil) rest | 17.8 |
| act | | | |
| CD4 lymphocyte none | 5.5 | KU-812 (Basophil) | 53.6 |
| | | PMA/ionomycin | |
| 2ry Th1/Th2/Tr1_anti-CD95 | 12.2 | CCD1106 (Keratinocytes) none | 48.6 |
| CH11 | | | |
| LAK cells rest | 15.3 | CCD1106 (Keratinocytes) | 25.5 |
| | | TNF alpha + IL-1beta | |
| LAK cells IL-2 | 24.7 | Liver cirrhosis | 3.4 |
| LAK cells IL-2 + IL-12 | 28.5 | Lupus kidney | 2.3 |
| LAK cells IL-2 + IFN gamma | 36.1 | NCI-H292 none | 21.6 |
| LAK cells IL-2 + IL-18 | 23.0 | NCI-H292 IL-4 | 31.4 |
| LAK cells PMA/ionomycin | 8.2 | NCI-H292 IL-9 | 24.7 |
| NK Cells IL-2 rest | 20.3 | NCI-H292 IL-13 | 15.2 |
| Two Way MLR 3 day | 18.6 | NCI-H292 IFN gamma | 18.2 |
| Two Way MLR 5 day | 17.9 | HPAEC none | 12.3 |
| Two Way MLR 7 day | 11.1 | HPAEC TNF alpha + IL-1beta | 15.7 |
| PBMC rest | 10.0 | Lung fibroblast none | 10.1 |
| PBMC PWM | 77.4 | Lung fibroblast TNF alpha + IL- | 11.1 |
| | | 1beta | |
| PBMC PHA-L | 32.3 | Lung fibroblast IL-4 | 24.5 |
| Ramos (B cell) none | 26.4 | Lung fibroblast IL-9 | 18.7 |
| Ramos (B cell) ionomycin | 100.0 | Lung fibroblast IL-13 | 17.6 |
| B lymphocytes PWM | 97.3 | Lung fibroblast IFN gamma | 26.4 |
| B lymphocytes CD40L and | 53.2 | Dermal fibroblast CCD1070 rest | 40.1 |
| IL-4 | | | |
| EOL-1 dbcAMP | 15.1 | Dermal fibroblast CCD1070 TNF | 59.9 |
| | | alpha | |
| EOL-1 dbcAMP | 27.5 | Dermal fibroblast CCD1070 IL- | 17.1 |
| PMA/ionomycin | | 1beta | |
| Dendritic cells none | 9.3 | Dermal fibroblast IFN gamma | 9.5 |
| Dendritic cells LPS | 7.2 | Dermal fibroblast IL-4 | 18.6 |
| Dendritic cells anti-CD40 | 7.3 | IBD Colitis 2 | 2.0 |
| Monocytes rest | 13.1 | IBD Crohn's | 2.0 |
| Monocytes LPS | 15.7 | Colon | 25.0 |
| Macrophages rest | 12.1 | Lung | 19.9 |
| Macrophages LPS | 9.9 | Thymus | 27.2 |
| HUVEC none | 20.4 | Kidney | 33.2 |
| HUVEC starved | 43.5 | | |

CNS_neurodegeneration_v1.0 Summary: Ag3309 This panel confirms the expression of the CG57693-01 gene at moderate levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3309 The CG57693-01 gene is expressed at high to moderate levels in the majority of samples on this panel, with highest expression detected in a gastric cancer cell line (CT=26.7). This gene encodes a protein with homology to Ser/Thr protein kinases. In general, expression of this gene appears to be higher in cancer cell lines than in the corresponding normal tissues. This overexpression is particularly evident in lung, colon, prostate and gastric cancer cell lines. Therefore, therapeutic modulation of the activity of this gene and it protein product may be of benefit in the treatment of lung, colon, prostate and gastric cancer. Furthermore, expression of this gene is significantly higher in fetal lung than in adult lung, suggesting that its expression may be used to distinguish fetal (CT=28.9) from adult lung (CT=32.1).

In addition, this gene is expressed at moderate levels throughout the CNS, including in amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at moderate levels in pancreas, adipose, adrenal gland, thyroid, pituitary gland, heart, skeletal muscle and liver. Therefore, therapeutic modulation of the activity of this gene may prove useful in the treatment of endocrine/metabolically related diseases, such as obesity and diabetes. Interestingly, expression of this gene is higher in fetal liver (CT=28.7) than in adult liver (CT=32.1), suggesting that expression of this gene can be used to distinguish adult from fetal liver.

Panel 4D Summary: Ag3309 This transcript is highly expressed in the PMA and ionomycin treated basophil cell line KU-812 (CT=28.2) and to a lesser extent in untreated KU-812 cells (CT=29.8). Therefore, antibody or small molecule therapies designed with the protein encoded for by this gene could block or inhibit inflammation or tissue damage due to basophil activation in response to asthma, allergies, hypersensitivity reactions, psoriasis, and viral infections.

This gene is also moderately expressed in Ramos B cells (CT=29.3) and the expression is highly stimulated by treatment with ionomycin and PWM (CTs=27). Expression of this transcript in B cells suggests that this gene may be involved in rheumatic disease including rheumatoid arthritis, lupus, osteoarthritis, and hyperproliferative B cell disorders.

In addition, treatment of small airway epithelium with TNFalpha+IL-1beta stimulated the expression of this transcript. Therefore, modulation of the expression or activity of the protein encoded by this transcript through the application of small molecule therapeutics may be useful in the treatment of asthma, COPD, and emphysema.

NOV54

Expression of NOV54/CG57707-01 was assessed using the primer-probe set Ag3312, described in Table AUA. Results of the RTQ-PCR runs are shown in Tables AUB, AUC and AUD.

TABLE AUA
Probe Name Ag3312

| Primers | Sequences | Length | Start Position | SEQ ID NO |
|---|---|---|---|---|
| Forward | 5′-gaccagctttgactatgaatgc-3′ | 22 | 624 | 425 |
| Probe | TET-5′-tgttcatcagactttaccctaccagtcgg-3′-TAMRA | 29 | 663 | 426 |
| Reverse | 5′-ggagttgaacgtatccactgaa-3′ | 22 | 693 | 427 |

TABLE AUB
CNS_neurodegeneration_v1.0

| | Rel. Exp. (%) Ag3312, Run | | Rel. Exp. (%) Ag3312, Run |
|---|---|---|---|
| Tissue Name | 210143316 | Tissue Name | 210143316 |
| AD 1 Hippo | 2.1 | Control (Path) 3 Temporal | 1.2 |
| | | Ctx | |
| AD 2 Hippo | 7.7 | Control (Path) 4 Temporal | 12.5 |
| | | Ctx | |
| AD 3 Hippo | 0.9 | AD 1 Occipital Ctx | 10.9 |
| AD 4 Hippo | 2.8 | AD 2 Occipital Ctx | 0.0 |
| | | (Missing) | |
| AD 5 Hippo | 100.0 | AD 3 Occipital Ctx | 2.2 |
| AD 6 Hippo | 10.3 | AD 4 Occipital Ctx | 6.6 |
| Control 2 Hippo | 17.1 | AD 5 Occipital Ctx | 54.7 |
| Control 4 Hippo | 3.1 | AD 6 Occipital Ctx | 12.9 |
| Control (Path) 3 Hippo | 2.5 | Control 1 Occipital Ctx | 5.2 |
| AD 1 Temporal Ctx | 5.1 | Control 2 Occipital Ctx | 84.7 |
| AD 2 Temporal Ctx | 6.7 | Control 3 Occipital Ctx | 6.9 |
| AD 3 Temporal Ctx | 1.2 | Control 4 Occipital Ctx | 0.9 |
| AD 4 Temporal Ctx | 5.3 | Control (Path) 1 Occipital | 66.0 |
| | | Ctx | |
| AD 5 Inf Temporal Ctx | 52.9 | Control (Path) 2 Occipital | 4.1 |
| | | Ctx | |
| AD 5 Sup Temporal Ctx | 14.0 | Control (Path) 3 Occipital | 3.7 |
| | | Ctx | |
| AD 6 Inf Temporal Ctx | 7.4 | Control (Path) 4 Occipital | 13.6 |
| | | Ctx | |
| AD 6 Sup Temporal Ctx | 8.1 | Control 1 Parietal Ctx | 3.0 |
| Control 1 Temporal Ctx | 5.6 | Control 2 Parietal Ctx | 7.9 |
| Control 2 Temporal Ctx | 27.0 | Control 3 Parietal Ctx | 16.8 |
| Control 3 Temporal Ctx | 8.6 | Control (Path) 1 Parietal | 79.0 |
| | | Ctx | |
| Control 3 Temporal Ctx | 1.4 | Control (Path) 2 Parietal | 11.8 |
| | | Ctx | |
| Control (Path) 1 Temporal | 25.7 | Control (Path) 3 Parietal | 4.6 |
| Ctx | | Ctx | |
| Control (Path) 2 Temporal | 21.8 | Control (Path) 4 Parietal | 34.9 |
| Ctx | | Ctx | |

TABLE AUC
General_screening_panel_v1.4

| | Rel. Exp. (%) Ag3312, Run | | Rel. Exp. (%) Ag3312, Run |
|---|---|---|---|
| Tissue Name | 215648085 | Tissue Name | 215648085 |
| Adipose | 11.8 | Renal ca. TK-10 | 38.2 |
| Melanoma* Hs688(A).T | 0.0 | Bladder | 8.5 |
| Melanoma* Hs688(B).T | 0.1 | Gastric ca. (liver met.) NCI- | 2.8 |
| | | N87 | |
| Melanoma* M14 | 0.0 | Gastric ca. KATO III | 0.0 |
| Melanoma* LOXIMVI | 0.3 | Colon ca. SW-948 | 0.0 |
| Melanoma* SK-MEL-5 | 3.6 | Colon ca. SW480 | 0.2 |
| Squamous cell carcinoma | 0.5 | Colon ca.* (SW480 met) | 0.4 |
| SCC-4 | | SW620 | |
| Testis Pool | 9.0 | Colon ca. HT29 | 0.0 |
| Prostate ca.* (bone met) | 0.0 | Colon ca. HCT-116 | 0.0 |
| PC-3 | | | |
| Prostate Pool | 2.0 | Colon ca. CaCo-2 | 11.6 |
| Placenta | 9.5 | Colon cancer tissue | 21.9 |
| Uterus Pool | 19.2 | Colon ca. SW1116 | 0.0 |
| Ovarian ca. OVCAR-3 | 29.3 | Colon ca. Colo-205 | 0.0 |
| Ovarian ca. SK-OV-3 | 8.4 | Colon ca. SW-48 | 0.0 |
| Ovarian ca. OVCAR-4 | 4.3 | Colon Pool | 59.9 |
| Ovarian ca. OVCAR-5 | 5.0 | Small Intestine Pool | 26.6 |
| Ovarian ca. IGROV-1 | 34.6 | Stomach Pool | 35.6 |
| Ovarian ca. OVCAR-8 | 2.2 | Bone Marrow Pool | 20.7 |
| Ovary | 8.9 | Fetal Heart | 18.6 |
| Breast ca. MCF-7 | 0.5 | Heart Pool | 20.7 |
| Breast ca. MDA-MB-231 | 0.0 | Lymph Node Pool | 55.1 |
| Breast ca. BT 549 | 18.4 | Fetal Skeletal Muscle | 8.2 |
| Breast ca. T47D | 4.3 | Skeletal Muscle Pool | 4.6 |
| Breast ca. MDA-N | 0.0 | Spleen Pool | 2.6 |
| Breast Pool | 36.9 | Thymus Pool | 16.6 |
| Trachea | 7.2 | CNS cancer (glio/astro) U87- | 2.4 |
| | | MG | |
| Lung | 10.5 | CNS cancer (glio/astro) U- | 3.6 |
| | | 118-MG | |
| Fetal Lung | 36.6 | CNS cancer (neuro; met) SK- | 6.9 |
| | | N-AS | |
| Lung ca. NCI-N417 | 11.0 | CNS cancer (astro) SF-539 | 0.9 |
| Lung ca. LX-1 | 0.0 | CNS cancer (astro) SNB-75 | 6.0 |
| Lung ca. NCI-H146 | 48.6 | CNS cancer (glio) SNB-19 | 41.5 |
| Lung ca. SHP-77 | 2.1 | CNS cancer (glio) SF-295 | 1.0 |
| Lung ca. A549 | 0.0 | Brain (Amygdala) Pool | 30.1 |
| Lung ca. NCI-H526 | 3.2 | Brain (cerebellum) | 100.0 |
| Lung ca. NCI-H23 | 10.4 | Brain (fetal) | 33.2 |
| Lung ca. NCI-H460 | 2.0 | Brain (Hippocampus) Pool | 31.9 |
| Lung ca. HOP-62 | 0.0 | Cerebral Cortex Pool | 51.1 |
| Lung ca. NCI-H522 | 3.1 | Brain (Substantia nigra) Pool | 50.7 |
| Liver | 0.7 | Brain (Thalamus) Pool | 43.2 |
| Fetal Liver | 3.0 | Brain (whole) | 55.1 |
| Liver ca. HepG2 | 0.0 | Spinal Cord Pool | 23.3 |
| Kidney Pool | 67.4 | Adrenal Gland | 8.8 |
| Fetal Kidney | 83.5 | Pituitary gland Pool | 0.0 |
| Renal ca. 786-0 | 0.0 | Salivary Gland | 1.8 |
| Renal ca. A498 | 2.0 | Thyroid (female) | 5.6 |
| Renal ca. ACHN | 0.2 | Pancreatic ca. CAPAN2 | 0.0 |
| Renal ca. UO-31 | 0.0 | Pancreas Pool | 35.6 |

TABLE AUD
Panel 4D

| | Rel. Exp. (%) Ag3312, Run | | Rel. Exp. (%) Ag3312, Run |
|---|---|---|---|
| Tissue Name | 164682846 | Tissue Name | 164682846 |
| Secondary Th1 act | 0.0 | HUVEC IL-1beta | 0.0 |
| Secondary Th2 act | 0.0 | HUVEC IFN gamma | 0.9 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + | 0.4 |
| | | IL-1beta | |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 0.0 | Bronchial epithelium TNF alpha + | 0.0 |
| | | IL1beta | |
| Primary Th2 rest | 0.0 | Small airway epithelium none | 0.0 |
| Primary Tr1 rest | 0.0 | Small airway epithelium TNF alpha + | 0.0 |
| | | IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNF alpha + | 0.0 |
| act | | IL-1beta | |
| CD8 lymphocyte act | 0.0 | Astrocytes rest | 0.0 |
| Secondary CD8 lymphocyte | 0.0 | Astrocytes TNF alpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 1.7 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.6 |
| | | TNF alpha + IL-1beta | |
| LAK cells IL-2 | 0.0 | Liver cirrhosis | 2.2 |
| LAK cells IL-2 + IL-12 | 2.2 | Lupus kidney | 3.0 |
| LAK cells IL-2 + IFN gamma | 0.0 | NCI-H292 none | 13.0 |
| LAK cells IL-2 + IL-18 | 0.0 | NCI-H292 IL-4 | 12.2 |
| LAK cells PMA/ionomycin | 0.0 | NCI-H292 IL-9 | 24.7 |
| NK Cells IL-2 rest | 0.0 | NCI-H292 IL-13 | 8.9 |
| Two Way MLR 3 day | 0.0 | NCI-H292 IFN gamma | 5.4 |
| Two Way MLR 5 day | 0.0 | HPAEC none | 0.0 |
| Two Way MLR 7 day | 0.0 | HPAEC TNF alpha + IL-1beta | 0.8 |
| PBMC rest | 0.0 | Lung fibroblast none | 0.0 |
| PBMC PWM | 0.0 | Lung fibroblast TNF alpha + IL- | 0.0 |
| | | 1beta | |
| PBMC PHA-L | 0.0 | Lung fibroblast IL-4 | 0.8 |
| Ramos (B cell) none | 0.0 | Lung fibroblast IL-9 | 0.8 |
| Ramos (B cell) ionomycin | 0.0 | Lung fibroblast IL-13 | 0.7 |
| B lymphocytes PWM | 1.3 | Lung fibroblast IFN gamma | 3.0 |
| B lymphocytes CD40L and | 0.9 | Dermal fibroblast CCD1070 rest | 2.2 |
| IL-4 | | | |
| EOL-1 dbcAMP | 0.0 | Dermal fibroblast CCD1070 TNF | 2.8 |
| | | alpha | |
| EOL-1 dbcAMP | 0.0 | Dermal fibroblast CCD1070 IL- | 0.0 |
| PMA/ionomycin | | 1beta | |
| Dendritic cells none | 0.0 | Dermal fibroblast IFN gamma | 8.1 |
| Dendritic cells LPS | 0.0 | Dermal fibroblast IL-4 | 0.0 |
| Dendritic cells anti-CD40 | 0.0 | IBD Colitis 2 | 11.0 |
| Monycytes rest | 0.0 | IBD Crohn's | 16.4 |
| Monocytes LPS | 0.0 | Colon | 100.0 |
| Macrophages rest | 8.2 | Lung | 69.3 |
| Macrophages LPS | 1.0 | Thymus | 47.3 |
| HUVEC none | 0.0 | Kidney | 23.7 |
| HUVEC starved | 0.0 | | |

CNS_neurodegeneration_v1.0 Summary: Ag3312 This panel confirms the expression of CG57707-01 gene at low to moderate levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3312 Expression of the CG57707-01 gene is highest in the cerebellum (CT=27.6). This gene is also expressed at moderate levels in all other regions of the central nervous system examined, including amygdala, hippocampus, substantia nigra, thalamus, cerebral cortex and spinal cord. The CG57707-01 gene encodes a protein with homology to the leucine-rich glioma-activated gene (LGI1). Recently, mutations in the LGI1 gene have been shown to cause autosomal-dominant partial epilepsy with auditory features (ref. 1). Based upon its homology to the LGI1 gene and significant exprssion in the brain, the CG57707-01 gene may also play a role in epilepsy or other central nervous system disorders such as Alzheimer's disease, Parkinson's disease, multiple sclerosis, schizophrenia and depression.

Strikingly, expression of this gene is primarily associated with the normal tissues on this panel. CG57707-01 gene expression appears to be down-regulated in pancreatic, colon, gastric, renal, lung, breast and prostate cancer cell lines as well as in most astrocytoma and glioma cell lines when compared to their respective normal tissues. This observation is consistent with what is known about the LGI1 gene, which was originally identified on the basis of its downregulation in malignant brain tumors (Ref. 1, 2). Therefore, therapeutic modulation of the activity of the CG57707-01 gene or its protein product, using small molecule drugs, antibodies or protein therapeutics, may be of benefit in the treatment of pancreatic, colon, gastric, renal, lung, breast, prostate, and CNS cancers.

Among tissues with metabolic or endocrine function, this gene is expressed at moderate levels in pancreas, adipose, adrenal gland, thyroid, skeletal muscle and heart. Therefore, therapeutic modulation of the activity of this gene may prove useful in the treatment of endocrine/metabolically related diseases, such as obesity and diabetes.

References:

1. Kalachikov S, Evgrafov O, Ross B, Winawer M, Barker Cummings C, Boneschi F M, Choi C, Morozov P, Das K, Teplitskaya E, Yu A, Cayanis E, Penchaszadeh G, Kottmann A H, Pedley T A, Hauser W A, Ottman R, Gilliam T C. Mutations in LGI1 cause autosomal-dominant partial epilepsy with auditory features. Nat Genet Jan. 28, 2002; [epub ahead of print]

The epilepsies are a common, clinically heterogeneous group of disorders defined by recurrent unprovoked seizures. Here we describe identification of the causative gene in autosomal-dominant partial epilepsy with auditory features (ADPEAF, MIM 600512), a rare form of idiopathic lateral temporal lobe epilepsy characterized by partial seizures with auditory disturbances. We constructed a complete, 4.2-Mb physical map across the genetically implicated disease gene region, identified 28 putative genes (FIG. 1) and resequenced all or part of 21 genes before identifying presumptive mutations in one copy of the leucine-rich, glioma-inactivated 1 gene (LGI1) in each of five families with ADPEAF. Previous studies have indicated that loss of both copies of LGI1 promotes glial tumor progression. We show that the expression pattern of mouse Lgi1 is predominantly neuronal and is consistent with the anatomic regions involved in temporal lobe epilepsy. Discovery of LGI1 as a cause of ADPEAF suggests new avenues for research on pathogenic mechanisms of idiopathic epilepsies.

PMID: 11810107

2. Chernova O B, Somerville R P, Cowell J K. A novel gene, LGI1, from 10q24 is rearranged and downregulated in malignant brain tumors. Oncogene Dec. 3, 1998;17(22):2873-81

Loss of heterozygosity for 10q23-26 is seen in over 80% of glioblastoma multiforme tumors. We have used a positional cloning strategy to isolate a novel gene, LGI1 (Leucine rich gene-Glioma Inactivated), which is rearranged as a result of the t(10;19)(q24;q13) balanced translocation in the T98G glioblastoma cell line lacking any normal chromosome 10. Rearrangement of the LGI1 gene was also detected in the A172 glioblastoma cell line and several glioblastoma tumors. These rearrangements lead to a complete absence of LGI1 expression in glioblastoma cells. The LGI1 gene encodes a protein with a calculated molecular mass of 60 kD and contains 3.5 leucine-rich repeats (LRR) with conserved flanking sequences. In the LRR domain, LGI1 has the highest homology with a number of transmembrane and extracellular proteins which function as receptors and adhesion proteins. LGI1 is predominantly expressed in neural tissues, especially in brain; its expression is reduced in low grade brain tumors and it is significantly reduced or absent in malignant gliomas. Its localization to the 10q24 region, and rearrangements or inactivation in malignant brain tumors, suggest that LGI1 is a candidate tumor suppressor gene involved in progression of glial tumors.

PMID: 9879993

Panel 4D Summary: Ag3312 The CG57707-01 gene is moderately expressed in samples derived from normal colon (CT=29.9), lung (CT=30.4), thymus (CT=30.9) and kidney (CT=31.9). Thus, the expression of this gene could be used to distinguish these tissues from the other samples in this panel. Expression of this gene in normal tissues is consistent with what is observed in General_screening_panel_v1.4.

Furthermore, expression of this gene is decreased in colon samples from patients with IBD colitis (CT=33) and Crohn's disease (CT=32.5) relative to normal colon. Therefore, therapeutic modulation of the activity of the protein encoded by this gene may be useful in the treatment of inflammatory bowel disease.

NOV55

Expression of NOV55/CG57306-01 was assessed using the primer-probe set Ag3157, described in Table AVA. Results of the RTQ-PCR runs are shown in Tables AVB, and AVC.

TABLE AVA
Probe Name Ag3157

| Primers | Sequences | Length | Start Position |
|---|---|---|---|
| Forward | 5′-tgtcatacagtcccagacattg-3′ | 22 | 1766 |
| Probe | TET-5′-ccttcttctcccttctcctcttcctt-3′-TAMRA | 26 | 1788 |
| Reverse | 5′-ggctggtctttacacacttgag-3′ | 22 | 1838 |

TABLE AVB
Panel 1.3D

| | Rel. Exp. (%) Ag3157, Run | | Rel. Exp. (%) Ag3157, Run |
|---|---|---|---|
| Tissue Name | 167994581 | Tissue Name | 167994581 |
| Liver adenocarcinoma | 0.0 | Kidney (fetal) | 48.6 |
| Pancreas | 0.2 | Renal ca. 786-0 | 0.0 |
| Pancreatic ca. CAPAN 2 | 1.1 | Renal ca. A498 | 0.2 |
| Adrenal gland | 0.0 | Renal ca. RXF 393 | 0.5 |
| Thyroid | 0.6 | Renal ca. ACHN | 0.0 |
| Salivary gland | 1.2 | Renal ca. UO-31 | 0.2 |
| Pituitary gland | 0.2 | Renal ca. TK-10 | 0.0 |
| Brain (fetal) | 2.9 | Liver | 0.2 |
| Brain (whole) | 5.5 | Liver (fetal) | 0.2 |
| Brain (amygdala) | 2.0 | Liver ca. (hepatoblast) | 0.0 |
| | | HepG2 | |
| Brain (cerebellum) | 2.0 | Lung | 0.8 |
| Brain (hippocampus) | 1.5 | Lung (fetal) | 0.0 |
| Brain (substantia nigra) | 3.5 | Lung ca. (small cell) LX-1 | 0.8 |
| Brain (thalamus) | 1.6 | Lung ca. (small cell) NCI- | 0.0 |
| | | H69 | |
| Cerebral Cortex | 1.6 | Lung ca. (s.cell var.) SHP- | 0.9 |
| | | 77 | |
| Spinal cord | 1.7 | Lung ca. (large cell)NCI- | 0.0 |
| | | H460 | |
| (glio/astro) U87-MG | 0.5 | Lung ca. (non-sm. cell) | 1.0 |
| | | A549 | |
| (glio/astro) U-118-MG | 0.0 | Lung ca. (non-s cell) NCI- | 1.4 |
| | | H23 | |
| astrocytoma SW1783 | 0.2 | Lung ca. (non-s cell) HOP- | 0.2 |
| | | 62 | |
| neuro*; met SK-N-AS | 0.5 | Lung ca. (non-s.cl) NCI- | 1.2 |
| | | H522 | |
| astrocytoma SF-539 | 0.5 | Lung ca. (squam.) SW 900 | 0.1 |
| astrocytoma SNB-75 | 0.4 | Lung ca. (squam.) NCI- | 0.0 |
| | | H596 | |
| glioma SNB-19 | 0.0 | Mammary gland | 0.0 |
| glioma U251 | 0.3 | Breast ca.* (pl.ef) MCF-7 | 1.0 |
| glioma SF-295 | 0.2 | Breast ca.* (pl.ef) MDA- | 0.2 |
| | | MB-231 | |
| Heart (fetal) | 0.4 | Breast ca.* (pl.ef) T47D | 1.8 |
| Heart | 0.2 | Breast ca. BT-549 | 0.8 |
| Skeletal muscle (fetal) | 0.6 | Breast ca. MDA-N | 0.4 |
| Skeletal muscle | 0.0 | Ovary | 0.0 |
| Bone marrow | 0.2 | Ovarian ca. OVCAR-3 | 0.4 |
| Thymus | 0.2 | Ovarian ca. OVCAR-4 | 0.0 |
| Spleen | 0.0 | Ovarian ca. OVCAR-5 | 2.7 |
| Lymph node | 0.0 | Ovarian ca. OVCAR-8 | 0.2 |
| Colorectal | 0.5 | Ovarian ca. IGROV-1 | 0.0 |
| Stomach | 0.0 | Ovarian ca* (ascites) SK- | 0.9 |
| | | OV-3 | |
| Small intestine | 0.0 | Uterus | 0.0 |
| Colon ca. SW480 | 0.2 | Placenta | 0.0 |
| Colon ca.* SW620(SW480 | 1.6 | Prostate | 0.5 |
| met) | | | |
| Colon ca. HT29 | 0.2 | Prostate ca.* (bone met)PC-3 | 0.9 |
| Colon ca. HCT-116 | 1.3 | Testis | 1.3 |
| Colon ca. CaCo-2 | 0.5 | Melanoma Hs688(A).T | 0.0 |
| Colon ca. tissue(ODO3866) | 0.0 | Melanoma* (met) | 0.2 |
| | | Hs688(B).T | |
| Colon ca. HCC-2998 | 2.6 | Melanoma UACC-62 | 0.0 |
| Gastric ca.* (liver met) NCI- | 0.3 | Melanoma M14 | 0.3 |
| N87 | | | |
| Bladder | 0.5 | Melanoma LOX IMVI | 0.0 |
| Trachea | 0.0 | Melanoma* (met) SK- | 1.5 |
| | | MEL-5 | |
| Kidney | 100.0 | Adipose | 0.4 |

TABLE AVC
Panel 4D

| | Rel. Exp. (%) | | Rel. Exp. (%) |
|---|---|---|---|
| | Ag3157, Run | | Ag3157, Run |
| Tissue Name | 164529941 | Tissue Name | 164529941 |
| Secondary Th1 act | 0.2 | HUVEC IL-1beta | 0.0 |
| Secondary Th2 act | 0.3 | HUVEC IFN gamma | 0.1 |
| Secondary Tr1 act | 1.1 | HUVEC TNF alpha + IFN gamma | 0.4 |
| Secondary Th1 rest | 0.7 | HUVEC TNF alpha + IL4 | 0.1 |
| Secondary Th2 rest | 0.4 | HUVEC IL-11 | 0.4 |
| Secondary Tr1 rest | 0.6 | Lung Microvascular EC none | 0.2 |
| Primary Th1 act | 0.4 | Lung Microvascular EC | 0.3 |
| | | TNF alpha + IL-1beta | |
| Primary Th2 act | 0.2 | Microvascular Dermal EC none | 0.1 |
| Primary Tr1 act | 0.8 | Microsvasular Dermal EC | 0.0 |
| | | TNF alpha + IL-1beta | |
| Primary Th1 rest | 1.1 | Bronchial epithelium TNF alpha + | 0.3 |
| | | IL1beta | |
| Primary Th2 rest | 1.1 | Small airway epithelium none | 0.4 |
| Primary Tr1 rest | 0.2 | Small airway epithelium | 0.2 |
| | | TNF alpha + IL-1beta | |
| CD45RA CD4 lymphocyte | 0.3 | Coronery artery SMC rest | 0.0 |
| act | | | |
| CD45RO CD4 lymphocyte | 0.4 | Coronery artery SMC TNF alpha + | 0.1 |
| act | | IL-1beta | |
| CD8 lymphocyte act | 0.0 | Astrocytes rest | 0.1 |
| Secondary CD8 lymphocyte | 0.2 | Astrocytes TNF alpha + IL-1beta | 0.2 |
| rest | | | |
| Secondary CD8 lymphocyte | 0.3 | KU-812 (Basophil) rest | 0.0 |
| act | | | |
| CD4 lymphocyte none | 0.2 | KU-812 (Basophil) | 0.5 |
| | | PMA/ionomycin | |
| 2ry Th1/Th2/Tr1_anti-CD95 | 1.4 | CCD1106 (Keratinocytes) none | 0.1 |
| CH11 | | | |
| LAK cells rest | 0.1 | CCD1106 (Keratinocytes) | 0.2 |
| | | TNF alpha + IL-1beta | |
| LAK cells IL-2 | 0.9 | Liver cirrhosis | 1.0 |
| LAK cells IL-2 + IL-12 | 0.7 | Lupus kidney | 4.4 |
| LAK cells IL-2 + IFN gamma | 0.4 | NCI-H292 none | 0.7 |
| LAK cells IL-2 + IL-18 | 1.3 | NCI-H292 IL-4 | 0.5 |
| LAK cells PMA/ionomycin | 0.0 | NCI-H292 IL-9 | 1.2 |
| NK Cells IL-2 rest | 0.3 | NCI-H292 IL-13 | 0.1 |
| Two Way MLR 3 day | 0.8 | NCI-H292 IFN gamma | 0.2 |
| Two Way MLR 5 day | 0.1 | HPAEC none | 0.2 |
| Two Way MLR 7 day | 0.3 | HPAEC TNF alpha + IL-1beta | 0.0 |
| PBMC rest | 0.0 | Lung fibroblast none | 0.0 |
| PBMC PWM | 1.7 | Lung fibroblast TNF alpha + IL- | 0.0 |
| | | 1beta | |
| PBMC PHA-L | 0.3 | Lung fibroblast IL-4 | 0.3 |
| Ramos (B cell) none | 0.9 | Lung fibroblast IL-9 | 0.1 |
| Ramos (B cell) ionomycin | 5.4 | Lung fibroblast IL-13 | 0.2 |
| B lymphocytes PWM | 1.1 | Lung fibroblast IFN gamma | 0.1 |
| B lymphocytes CD40L and | 2.2 | Dermal fibroblast CCD1070 rest | 0.0 |
| IL-4 | | | |
| EOL-1 dbcAMP | 0.0 | Dermal fibroblast CCD1070 TNF | 1.3 |
| | | alpha | |
| EOL-1 dbcAMP | 0.5 | Dermal fibroblast CCD1070 IL- | 0.0 |
| PMA/ionomycin | | 1beta | |
| Dendritic cells none | 0.0 | Dermal fibroblast IFN gamma | 0.1 |
| Dendritic cells LPS | 0.2 | Dermal fibroblast IL-4 | 0.2 |
| Dendritic cells anti-CD40 | 0.1 | IBD Colitis 2 | 0.0 |
| Monocytes rest | 0.0 | IBD Crohn's | 0.0 |
| Monocytes LPS | 0.7 | Colon | 0.6 |
| Macrophages rest | 0.1 | Lung | 1.1 |
| Macrophages LPS | 0.0 | Thymus | 100.0 |
| HUVEC none | 0.1 | Kidney | 0.3 |
| HUVEC starved | 1.0 | | |

Panel 1.3D Summary: Ag3157 Expression of the CG57306-01 gene is highest in samples derived from normal kidney (CT=29.7) and fetal kidney (CT=30.8). Thus, the expression of this gene could be used to distinguish kidney from the other samples in the panel.

The CG57306-01 gene encodes a variant of anion exchanger AE4, which is expressed primarily in the kidney and is predicted to play a role in sodium bicarbonate exchange (ref. 1). Mutations in sodium bicarbonate transporters have been shown to be associated with renal tubular acidosis (RTA) (ref. 2-3). Thus, therapies designed with the protein encoded for by this gene may potentially play a role in the identification and treatment of RTA or other kidney related diseases.

This transcript is also expressed at low levels in samples derived from brain, including whole adult brain (CT=33.9) and substantia nigra (CT=34.5). Therefore, this gene may play a role in central nervous system disorders such as Alzheimer's disease, Parkinson's disease, epilepsy, multiple sclerosis, schizophrenia and depression.

References:

1. Parker M D, Ourmozdi E P, Tanner M J. Human BTR1, a new bicarbonate transporter superfamily member and human AE4 from kidney. Biochem Biophys Res Commun Apr. 20, 2001;282(5):1103-9

We report the cloning, characterization, and chromosomal localization of two novel human members of the bicarbonate transporter superfamily, BTR1 (Bicarbonate Transporter Related protein-1) and AE4 (Anion Exchange protein 4). BTR1 is a novel mammalian protein. The BTR1 gene maps to chromosome 20p12 and encodes a 100 kDa protein predominantly expressed in the kidney, salivary glands, testis, thyroid glands, and trachea. The AE4 gene maps to chromosome 5q23-31 and encodes a 104 kDa protein expressed mainly in the kidney. Human AE4 shares 84% identity with the recently reported rabbit AE4, a sodium independent, Cl(−)/HCO(−)(3) exchanger located on the apical membrane of beta-intercalated kidney cells. Copyright 2001 Academic Press.

PMID: 11302728

2. Soleimani M, Burnham C E. Physiologic and molecular aspects of the Na+:HCO3-cotransporter in health and disease processes. Kidney Int 2000 February;57(2):371-84

Approximately 80% of the filtered load of HCO3- is reabsorbed in the proximal tubule via a process of active acid secretion by the luminal membrane. The major mechanism for the transport of HCO3- across the basolateral membrane is via the electrogenic Na+:3HCO3-cotransporter (NBC). Recent molecular cloning experiments have identified the existence of three NBC isoforms (NBC-1, NBC-2, and NBC-3).1 Functional and molecular studies indicate the presence of all three NBC isoforms in the kidney. All are presumed to mediate the cotransport of Na+ and HCO3- under normal conditions and may be functionally altered in certain pathophysiologic states. Specifically, NBC-I may be up regulated in metabolic acidosis and potassium depletion and in response to glucocorticoid excess and may be down regulated in response to HCO3- loading or alkalosis. Recent studies provide molecular evidence indicating the expression of NBC-1 in pancreatic duct cells. NBC is activated by cystic fibrosis transmembrane conductance regulator (CFTR) and plays an important role in HCO3- secretion in the agonist-stimulated state in pancreatic duct cells. The purpose of this review is to summarize recent functional and molecular studies on the regulation of NBCs in physiologic and pathophysiologic states. Possible signals responsible for the regulation of NBCs in these conditions are examined. Furthermore, the possible role of this transporter in acid-base disorders (such as proximal renal tubular acidosis) is discussed.

PMID: 10652014

3. Rodriguez-Soriano J., 2000, New insights into the pathogenesis of renal tubular acidosis—from functional to molecular studies. Pediatr Nephrol October; 14(12):1121-36.

The diagnosis and classification of renal tubular acidosis (RTA) have traditionally been made on the basis of functional studies. On these grounds, RTA has been separated into three main categories: (1) proximal RTA, or type 2; (2) distal RTA, or type l; and (3) hyperkalemic RTA, or type 4. In recent years significant advances have been made in our understanding of the subcellular mechanisms involved in renal bicarbonate (HCO3-) and H+ transport. Application of molecular biology techniques has also opened a completely new perspective to the understanding of the pathophysiology of inherited cases of RTA. Mutations in the gene SLC4A4, encoding Na+-HCO3- cotransporter (NBC-1), have been found in proximal RTA with ocular abnormalities; in the gene SLC4AI, encoding Cl(−)—HCO3- exchanger (AEI), in autosomal dominant distal RTA; in the gene ATP6BI, encoding B1 subunit of H+-ATPase, in autosomal recessive distal RTA with sensorineural deafness; and in the gene CA2, encoding carbonic anhydrase II, in autosomal recessive osteopetrosis. Syndromes of aldosterone resistance have been also characterized molecularly and mutations in the gene MLR, encoding mineralocorticoid receptor, and in the genes SNCC1A, SNCC1B, and SCNN1G, encoding subunits of the epithelial Na+ channel, have been found in dominant and recessive forms of pseudohypoaldosteronism type 1, respectively. It can be concluded that, although functional studies are still necessary, a new molecular era in the understanding of disorders of renal acidification has arrived.

PMID: 11045400

Panel 4D Summary: Ag3157 Expression of the CG57306-01 gene is highest in thymus (CT=27.9). Therefore, expression of this gene can be used to distinguish thymus from the other samples on this panel. The putative anion exchanger encoded for by this gene could therefore play an important role in T cell development. Furthermore, small molecule drugs or antibody therapeutics designed against the protein encoded for by this gene could be utilized to modulate immune function (T cell development) and be important for organ transplant, AIDS treatment or post chemotherapy immune reconstitiution.

Panel 5 Islet Summary: Ag3157 Expression of this gene is low/undetectable (CTs>35) across all of the samples on this panel (data not shown).

NOV56

Expression of NOV56/CG57348-01 was assessed using the primer-probe set Ag3763, described in Table AWA. Results of the RTQ-PCR runs are shown in Tables AWB, AWC and AWD.

TABLE AWA
Probe Name Ag3763

| Primers | Sequences | Length | Start Position | SEQ ID NO |
|---|---|---|---|---|
| Forward | 5′-gatggggagagcgtatttacc-3′ | 21 | 274 | 428 |
| Probe | TET-5′-tcaaagatctattcctacatgagcccga-3′-TAMRA | 28 | 301 | 429 |
| Reverse | 5′-aaacgcattccagagcattt-3′ | 20 | 331 | 430 |

TABLE AWB
CNS_neurodegeneration_v1.0

| | Rel. Exp. (%) | | Rel. Exp. (%) |
|---|---|---|---|
| | Ag3763, Run | | Ag3763, Run |
| Tissue Name | 211175130 | Tissue Name | 211175130 |
| AD 1 Hippo | 19.8 | Control (Path) 3 Temporal | 17.7 |
| | | Ctx | |
| AD 2 Hippo | 46.7 | Control (Path) 4 Temporal | 59.9 |
| | | Ctx | |
| AD 3 Hippo | 19.6 | AD 1 Occipital Ctx | 27.9 |
| AD 4 Hippo | 13.2 | AD 2 Occipital Ctx | 0.0 |
| | | (Missing) | |
| AD 5 hippo | 86.5 | AD 3 Occipital Ctx | 20.7 |
| AD 6 Hippo | 92.0 | AD 4 Occipital Ctx | 20.9 |
| Control 2 Hippo | 46.7 | AD 5 Occipital Ctx | 39.8 |
| Control 4 Hippo | 25.2 | AD 6 Occipital Ctx | 44.8 |
| Control (Path) 3 Hippo | 12.0 | Control 1 Occipital Ctx | 15.4 |
| AD 1 Temporal Ctx | 45.7 | Control 2 Occipital Ctx | 49.0 |
| AD 2 Temporal Ctx | 47.3 | Control 3 Occipital Ctx | 27.7 |
| AD 3 Temporal Ctx | 12.2 | Control 4 Occipital Ctx | 14.5 |
| AD 4 Temporal Ctx | 24.3 | Control (Path) 1 Occipital | 96.6 |
| | | Ctx | |
| AD 5 Inf Temporal Ctx | 100.0 | Control (Path) 2 Occipital | 28.9 |
| | | Ctx | |
| AD 5 SupTemporal Ctx | 87.1 | Control (Path) 3 Occipital | 5.9 |
| | | Ctx | |
| AD 6 Inf Temporal Ctx | 61.1 | Control (Path) 4 Occipital | 35.1 |
| | | Ctx | |
| AD 6 Sup Temporal Ctx | 88.9 | Control 1 Parietal Ctx | 18.0 |
| Control 1 Temporal Ctx | 12.5 | Control 2 Parietal Ctx | 66.0 |
| Control 2 Temporal Ctx | 48.6 | Control 3 Parietal Ctx | 34.9 |
| Control 3 Temporal Ctx | 30.6 | Control (Path) 1 Parietal | 77.9 |
| | | Ctx | |
| Control 4 Temporal Ctx | 13.2 | Control (Path) 2 Parietal | 45.1 |
| | | Ctx | |
| Control (Path) 1 Temporal | 85.3 | Control (Path) 3 Parietal | 6.6 |
| Ctx | | Ctx | |
| Control (Path) 2 Temporal | 65.1 | Control (Path) 4 Parietal | 52.5 |
| Ctx | | Ctx | |

TABLE AWC
General_screening_panel_v1.4

| | Rel. Exp. (%) | | Rel. Exp. (%) |
|---|---|---|---|
| | Ag3763, Run | | Ag3763, Run |
| Tissue Name | 216557719 | Tissue Name | 216557719 |
| Adipose | 7.7 | Renal ca. TK-10 | 24.8 |
| Melanoma* Hs688(A).T | 31.4 | Bladder | 17.6 |
| Melanoma* Hs688(B).T | 27.7 | Gastric ca. (liver met.) NCI- | 30.4 |
| | | N87 | |
| Melanoma* M14 | 30.8 | Gastric ca. KATO III | 42.0 |
| Melanoma* LOXIMVI | 29.1 | Colon ca. SW-948 | 7.7 |
| Melanoma* SK-MEL-5 | 19.2 | Colon ca. SW480 | 40.6 |
| Squamous cell carcinoma | 16.4 | Colon ca.* (SW480 met) | 54.3 |
| SCC-4 | | SW620 | |
| Testis Pool | 6.4 | Colon ca. HT29 | 18.9 |
| Prostate ca.* (bone met) | 52.9 | Colon ca. HCT-116 | 58.2 |
| PC-3 | | | |
| Prostate Pool | 23.8 | Colon ca. CaCo-2 | 21.6 |
| Placenta | 9.3 | Colon cancer tissue | 21.8 |
| Uterus Pool | 7.9 | Colon ca. SW1116 | 11.2 |
| Ovarian ca. OVCAR-3 | 19.3 | Colon ca. Colo-205 | 10.8 |
| Ovarian ca. SK-OV-3 | 35.4 | Colon ca. SW-48 | 11.2 |
| Ovarian ca. OVCAR-4 | 13.7 | Colon Pool | 8.8 |
| Ovarian ca. OVCAR-5 | 35.8 | Small Intestine Pool | 16.0 |
| Ovarian ca. IGROV-1 | 31.9 | Stomach Pool | 11.0 |
| Ovarian ca. OVCAR-8 | 52.1 | Bone Marrow Pool | 6.2 |
| Ovary | 4.5 | Fetal Heart | 6.0 |
| Breast ca. MCF-7 | 37.9 | Heart Pool | 4.6 |
| Breast ca. MDA-MB-231 | 36.9 | Lymph Node Pool | 14.1 |
| Breast ca. BT 549 | 44.1 | Fetal Skeletal Muscle | 5.5 |
| Breast ca. T47D | 100.0 | Skeletal Muscle Pool | 12.2 |
| Breast ca. MDA-N | 12.2 | Spleen Pool | 12.3 |
| Breast Pool | 8.7 | Thymus Pool | 14.4 |
| Trachea | 16.3 | CNS cancer (glio/astro) U87- | 25.0 |
| | | MG | |
| Lung | 7.6 | CNS cancer (glio/astro) U- | 52.9 |
| | | 118-MG | |
| Fetal Lung | 15.0 | CNS cancer (neuro; met) SK- | 42.9 |
| | | N-AS | |
| Lung ca. NCI-N417 | 14.8 | CNS cancer (astro) SF-539 | 20.9 |
| Lung ca. LX-1 | 47.3 | CNS cancer (astro) SNB-75 | 18.9 |
| Lung ca. NCI-H146 | 12.0 | CNS cancer (glio) SNB-19 | 32.1 |
| Lung ca. SHP-77 | 47.0 | CNS cancer (glio) SF-295 | 63.3 |
| Lung ca. A549 | 23.8 | Brain (Amygdala) Pool | 7.5 |
| Lung ca. NCI-H526 | 17.1 | Brain (cerebellum) | 10.1 |
| Lung ca. NCI-H23 | 64.6 | Brain (fetal) | 12.6 |
| Lung ca. NCI-H460 | 26.2 | Brain (Hippocampus) Pool | 8.7 |
| Lung ca. HOP-62 | 8.8 | Cerebral Cortex Pool | 11.5 |
| Lung ca. NCI-H522 | 42.9 | Brain (Substantia nigra) Pool | 10.8 |
| Liver | 0.6 | Brain (Thalamus) Pool | 14.5 |
| Fetal Liver | 18.6 | Brain (whole) | 13.3 |
| Liver ca. HepG2 | 10.1 | Spinal Cord Pool | 13.3 |
| Kidney Pool | 12.2 | Adrenal Gland | 9.9 |
| Fetal Kidney | 4.8 | Pituitary gland Pool | 3.5 |
| Renal ca. 786-0 | 18.2 | Salivary Gland | 14.4 |
| Renal ca. A498 | 10.4 | Thyroid (female) | 11.3 |
| Renal ca. ACHN | 17.1 | Pancreatic ca. CAPAN2 | 17.7 |
| Renal ca. UO-31 | 20.3 | Pancreas Pool | 21.5 |

TABLE AWD
Panel 4.1D

| | Rel. Exp. (%) | | Rel. Exp. (%) |
|---|---|---|---|
| | Ag3763, Run | | Ag3763, Run |
| Tissue Name | 170069604 | Tissue Name | 170069604 |
| Secondary Th1 act | 72.7 | HUVEC IL-1beta | 56.3 |
| Secondary Th2 act | 73.2 | HUVEC IFN gamma | 45.1 |
| Secondary Tr1 act | 58.2 | HUVEC TNF alpha + IFN gamma | 25.3 |
| Secondary Th1 rest | 8.7 | HUVEC TNF alpha + IL4 | 37.4 |
| Secondary Th2 rest | 28.1 | HUVEC IL-11 | 26.4 |
| Secondary Tr1 rest | 14.7 | Lung Microvascular EC none | 48.0 |
| Primary Th1 act | 52.1 | Lung Microvascular EC | 27.2 |
| | | TNF alpha + IL-1beta | |
| Primary Th2 act | 52.1 | Microvascular Dermal EC none | 48.3 |
| Primary Tr1 act | 52.5 | Microsvasular Dermal EC | 26.8 |
| | | TNF alpha + IL-1beta | |
| Primary Th1 rest | 28.1 | Bronchial epithelium TNF alpha + | 24.5 |
| | | IL1beta | |
| Primary Th2 rest | 17.9 | Small airway epithelium none | 17.3 |
| Primary Tr1 rest | 36.9 | Small airway epithelium TNF | 27.9 |
| | | alpha + IL-1beta | |
| CD45RA CD4 lymphocyte | 43.5 | Coronery artery SMC rest | 26.8 |
| act | | | |
| CD45RO CD4 lymphocyte | 43.2 | Coronery artery SMC TNF alpha + | 15.9 |
| act | | IL-1beta | |
| CD8 lymphocyte act | 48.0 | Astrocytes rest | 29.9 |
| Secondary CD8 lymphocyte | 37.9 | Astrocytes TNF alpha + IL-1beta | 23.5 |
| rest | | | |
| Secondary CD8 lymphocyte | 32.5 | KU-812 (Basophil) rest | 46.7 |
| act | | | |
| CD4 lymphocyte none | 11.5 | KU-812 (Basophil) | 60.3 |
| | | PMA/ionomycin | |
| 2ry Th1/Th2/Tr1_anti-CD95 | 23.8 | CCD1106 (Keratinocytes) none | 34.9 |
| CH11 | | | |
| LAK cells rest | 29.7 | CCD1106 (Keratinocytes) | 29.5 |
| | | TNF alpha + IL-1beta | |
| LAK cells IL-2 | 38.7 | Liver cirrhosis | 23.7 |
| LAK cells IL-2 + IL-12 | 55.9 | NCI-H292 none | 20.7 |
| LAK cells IL-2 + IFN gamma | 46.3 | NCI-H292 IL-4 | 28.7 |
| LAK cells IL-2 + IL-18 | 43.8 | NCI-H292 IL-9 | 43.8 |
| LAK cells PMA/ionomycin | 30.1 | NCI-H292 IL-13 | 27.7 |
| NK Cells IL-2 rest | 57.0 | NCI-H292 IFN gamma | 48.3 |
| Two Way MLR 3 day | 26.1 | HPAEC none | 25.3 |
| Two Way MLR 5 day | 37.6 | HPAEC TNF alpha + IL-1beta | 26.4 |
| Two Way MLR 7 day | 39.8 | Lung fibroblast none | 41.8 |
| PBMC rest | 19.2 | Lung fibroblast TNF alpha + IL- | 15.1 |
| | | 1beta | |
| PBMC PWM | 38.4 | Lung fibroblast IL-4 | 39.0 |
| PBMC PHA-L | 39.5 | Lung fibroblast IL-9 | 43.8 |
| Ramos (B cell) none | 80.7 | Lung fibroblast IL-13 | 44.1 |
| Ramos (B cell) ionomycin | 76.8 | Lung fibroblast IFN gamma | 35.8 |
| B lymphocytes PWM | 43.2 | Dermal fibroblast CCD1070 rest | 95.9 |
| B lymphocytes CD40L and | 32.5 | Dermal fibroblast CCD1070 TNF | 90.8 |
| IL-4 | | alpha | |
| EOL-1 dbcAMP | 29.3 | Dermal fibroblast CCD1070 IL- | 45.7 |
| | | 1beta | |
| EOL-1 dbcAMP | 48.0 | Dermal fibroblast IFN gamma | 19.6 |
| PMA/ionomycin | | | |
| Dendritic cells none | 44.1 | Dermal fibroblast IL-4 | 66.0 |
| Dendritic cells LPS | 8.9 | Dermal Fibroblast rest | 35.8 |
| Dendritic cells anti-CD40 | 29.7 | Neutrophils TNFa+LPS | 9.8 |
| Monocytes rest | 25.3 | Neutrophils rest | 17.6 |
| Monocytes LPS | 18.0 | Colon | 13.2 |
| Macrophages rest | 27.9 | Lung | 20.6 |
| Macrophages LPS | 9.3 | Thymus | 100.0 |
| HUVEC none | 48.3 | Kidney | 19.9 |
| HUVEC starved | 31.4 | | |

CNS_neurodegeneration_v1.0 Summary: Ag3763 This panel confirms the expression of the CG57348-01 gene at low to moderate levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3763 Expression of the CG57348-01 gene is highest in a breast cancer cell line (CT=29.8). Interestingly, this gene appears to be more highly expressed in a number of cancer cell lines than in normal tissues. Expression of this gene appears to be upregulated in CNS, colon, gastric, lung, breast and ovarian cancer cell lines. Therefore, therapeutic modulation of the activity of this gene or its protein product, using small molecule drugs, antibodies or protein therapeutics, may be of benefit in the treatment of CNS, colon, gastric, lung, breast, and ovarian cancer.

In addition, this gene is expressed at low levels in all regions of the central nervous system examined, including in amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

This gene is also expressed at low levels in a number of tissues with metabolic or endocrine function, including adipose, pancreas, adrenal gland, thyroid, gastrointestinal tract, skeletal muscle, heart and liver. Therefore, therapeutic modulation of the activity of this gene may prove useful in the treatment of endocrine/metabolically related diseases, such as obesity and diabetes. Interestingly, this gene is expressed at higher levels in fetal liver (CT=32.2) when compared to adult liver (CT=37.2), suggesting that expression of this gene can be used to distinguish fetal from adult liver and may be of benefit in the treatment of liver degeneration.

Panel 4.1D Summary: Ag3763 This gene is expressed at low to moderate levels in a wide range of cell types of significance to the immune response in health and disease. These cells include T-cells, B-cells, endothelial cells, macrophages, monocytes, dendritic cells, basophils, eosinophils and peripheral blood mononuclear cells, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues.

This pattern is in agreement with the expression profile in General_screening_panel_v 1.4 and also suggests a role for the gene product in cell survival and proliferation.

Therefore, therapeutic modulation of the activity of this gene or its protein product may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

NOV60

Expression of gene NOV60/CG57574-01 was assessed using the primer-probe set Ag3289, described in Table AXA.

TABLE AXA
Probe Name Ag3289

| Primers | Sequences | Length | Start Position | SEQ ID NO |
|---|---|---|---|---|
| Forward | 5′-accagcctgtcaactactcctt-3′ | 22 | 419 | 431 |
| Probe | TET-5′-ccactccacctacttggtgaaccagg-3′-TAMRA | 26 | 453 | 432 |
| Reverse | 5′-ggaaattgacactctggtcaaa-3′ | 22 | 484 | 433 |

CNS_neurodegeneration_v1.0 Summary: Ag3289 Expression of this gene is low/undetectable (CTs>35) across all of the samples on this panel (data not shown).

General_screening_panel_v1.4 Summary: Ag3289 Expression of this gene is low/undetectable (CTs>35) across all of the samples on this panel (data not shown). The amp plot indicates that there were experimental difficulties with this run.

Panel 4D Summary: Ag3289 Expression of this gene is low/undetectable (CTs>35) across all of the samples on this panel (data not shown).

NOV61

Expression of NOV61/CG57505-01 was assessed using the primer-probe set Ag3259, described in Table AYA. Results of the RTQ-PCR runs are shown in Tables AYB, AYC and AYD.

TABLE AYA
Probe Name Ag3259

| Primers | Sequences | Length | Start Position | SEQ ID NO |
|---|---|---|---|---|
| Forward | 5′-catcgagacccagagtaaagc-3′ | 21 | 604 | 434 |
| Probe | TET-5′-catgacaatgctcaccattgaacagt-3′-TAMRA | 26 | 625 | 435 |
| Reverse | 5′-tttcattttctgaatggcaaac-3′ | 22 | 667 | 436 |

TABLE AYB
CNS_neurodegeneration_v1.0

| | Rel. Exp. (%) | | Rel. Exp. (%) |
|---|---|---|---|
| | Ag3259, Run | | Ag3259, Run |
| Tissue Name | 209990878 | Tissue Name | 209990878 |
| AD 1 Hippo | 7.1 | Control (Path) 3 Temporal | 3.5 |
| | | Ctx | |
| AD 2 Hippo | 14.9 | Control (Path) 4 Temporal | 16.8 |
| | | Ctx | |
| AD 3 Hippo | 5.8 | AD 1 Occipital Ctx | 8.3 |
| AD 4 Hippo | 4.3 | AD 2 Occipital Ctx | 0.0 |
| | | (Missing) | |
| AD 5 hippo | 100.0 | AD 3 Occipital Ctx | 6.6 |
| AD 6 Hippo | 6.1 | AD 4 Occipital Ctx | 9.7 |
| Control 2 Hippo | 11.9 | AD 5 Occipital Ctx | 6.8 |
| Control 4 Hippo | 7.5 | AD 6 Occipital Ctx | 27.2 |
| Control (Path) 3 Hippo | 4.3 | Control 1 Occipital Ctx | 2.4 |
| AD 1 Temporal Ctx | 7.7 | Control 2 Occipital Ctx | 30.1 |
| AD 2 Temporal Ctx | 17.4 | Control 3 Occipital Ctx | 8.0 |
| AD 3 Temporal Ctx | 5.7 | Control 4 Occipital Ctx | 4.8 |
| AD 4 Temporal Ctx | 10.6 | Control (Path) 1 Occipital | 51.1 |
| | | Ctx | |
| AD 5 Inf Temporal Ctx | 67.8 | Control (Path) 2 Occipital | 4.8 |
| | | Ctx | |
| AD 5 SupTemporal Ctx | 33.9 | Control (Path) 3 Occipital | 0.6 |
| | | Ctx | |
| AD 6 Inf Temporal Ctx | 23.7 | Control (Path) 4 Occipital | 10.7 |
| | | Ctx | |
| AD 6 Sup Temporal Ctx | 17.2 | Control 1 Parietal Ctx | 3.2 |
| Control 1 Temporal Ctx | 3.9 | Control 2 Parietal Ctx | 9.7 |
| Control 2 Temporal Ctx | 23.8 | Control 3 Parietal Ctx | 6.9 |
| Control 3 Temporal Ctx | 4.2 | Control (Path) 1 Parietal | 38.4 |
| | | Ctx | |
| Control 4 Temporal Ctx | 4.4 | Control (Path) 2 Parietal | 11.0 |
| | | Ctx | |
| Control (Path) 1 Temporal | 37.4 | Control (Path) 3 Parietal | 2.6 |
| Ctx | | Ctx | |
| Control (Path) 2 Temporal | 9.3 | Control (Path) 4 Parietal | 30.1 |
| Ctx | | Ctx | |

TABLE AYC
General_screening_panel_v1.4

| | Rel. Exp. (%) | | Rel. Exp. (%) |
|---|---|---|---|
| | Ag3259, Run | | Ag3259, Run |
| Tissue Name | 214694855 | Tissue Name | 214694855 |
| Adipose | 4.2 | Renal ca. TK-10 | 30.1 |
| Melanoma* Hs688(A).T | 6.8 | Bladder | 10.8 |
| Melanoma* Hs688(B).T | 5.1 | Gastric ca. (liver met.) NCI- | 100.0 |
| | | N87 | |
| Melanoma* M14 | 31.9 | Gastric ca. KATO III | 25.5 |
| Melanoma* LOXIMVI | 21.6 | Colon ca. SW-948 | 2.2 |
| Melanoma* SK-MEL-5 | 17.3 | Colon ca. SW480 | 52.5 |
| Squamous cell carcinoma | 14.9 | Colon ca.* (SW480 met) | 42.9 |
| SCC-4 | | SW620 | |
| Testis Pool | 8.0 | Colon ca. HT29 | 24.0 |
| Prostate ca.* (bone met) | 14.3 | Colon ca. HCT-116 | 15.4 |
| PC-3 | | | |
| Prostate Pool | 4.2 | Colon ca. CaCo-2 | 88.9 |
| Placenta | 6.9 | Colon cancer tissue | 23.3 |
| Uterus Pool | 1.2 | Colon ca. SW1116 | 3.0 |
| Ovarian ca. OVCAR-3 | 9.7 | Colon ca. Colo-205 | 8.0 |
| Ovarian ca. SK-OV-3 | 6.1 | Colon ca. SW-48 | 3.7 |
| Ovarian ca. OVCAR-4 | 6.4 | Colon Pool | 6.4 |
| Ovarian ca. OVCAR-5 | 62.4 | Small Intestine Pool | 4.5 |
| Ovarian ca. IGROV-1 | 15.8 | Stomach Pool | 3.7 |
| Ovarian ca. OVCAR-8 | 4.4 | Bone Marrow Pool | 2.6 |
| Ovary | 6.2 | Fetal Heart | 12.2 |
| Breast ca. MCF-7 | 94.6 | Heart Pool | 1.0 |
| Breast ca. MDA-MB-231 | 32.5 | Lymph Node Pool | 5.8 |
| Breast ca. BT 549 | 63.7 | Fetal Skeletal Muscle | 4.2 |
| Breast ca. T47D | 34.4 | Skeletal Muscle Pool | 5.1 |
| Breast ca. MDA-N | 17.4 | Spleen Pool | 9.7 |
| Breast Pool | 8.5 | Thymus Pool | 14.4 |
| Trachea | 10.3 | CNS cancer (glio/astro) U87- | 42.9 |
| | | MG | |
| Lung | 2.0 | CNS cancer (glio/astro) U- | 17.3 |
| | | 118-MG | |
| Fetal Lung | 21.2 | CNS cancer (neuro; met) SK- | 26.8 |
| | | N-AS | |
| Lung ca. NCI-N417 | 5.4 | CNS cancer (astro) SF-539 | 14.9 |
| Lung ca. LX-1 | 48.3 | CNS cancer (astro) SNB-75 | 22.1 |
| Lung ca. NCI-H146 | 9.1 | CNS cancer (glio) SNB-19 | 3.7 |
| Lung ca. SHP-77 | 17.3 | CNS cancer (glio) SF-295 | 24.5 |
| Lung ca. A549 | 13.3 | Brain (Amygdala) Pool | 6.6 |
| Lung ca. NCI-H526 | 4.4 | Brain (cerebellum) | 14.0 |
| Lung ca. NCI-H23 | 39.0 | Brain (fetal) | 24.5 |
| Lung ca. NCI-H460 | 7.6 | Brain (Hippocampus) Pool | 8.1 |
| Lung ca. HOP-62 | 9.9 | Cerebral Cortex Pool | 11.9 |
| Lung ca. NCI-H522 | 16.2 | Brain (Substantia nigra) Pool | 7.4 |
| Liver | 0.9 | Brain (Thalamus) Pool | 6.3 |
| Fetal Liver | 5.0 | Brain (whole) | 8.2 |
| Liver ca. HepG2 | 33.0 | Spinal Cord Pool | 5.8 |
| Kidney Pool | 3.5 | Adrenal Gland | 24.8 |
| Fetal Kidney | 15.2 | Pituitary gland Pool | 5.3 |
| Renal ca. 786-0 | 20.2 | Salivary Gland | 4.4 |
| Renal ca. A498 | 17.2 | Thyroid (female) | 2.1 |
| Renal ca. ACHN | 17.3 | Pancreatic ca. CAPAN2 | 18.6 |
| Renal ca. UO-31 | 46.3 | Pancreas Pool | 10.4 |

TABLE AYD
Panel 4D

| | Rel. Exp. (%) | | Rel. Exp. (%) |
|---|---|---|---|
| | Ag3259, Run | | Ag3259, Run |
| Tissue Name | 164537290 | Tissue Name | 164537290 |
| Secondary Th1 act | 22.5 | HUVEC IL-1beta | 13.5 |
| Secondary Th2 act | 20.6 | HUVEC IFN gamma | 50.0 |
| Secondary Tr1 act | 23.3 | HUVEC TNF alpha + IFN gamma | 21.6 |
| Secondary Th1 rest | 7.7 | HUVEC TNF alpha + IL4 | 15.4 |
| Secondary Th2 rest | 9.9 | HUVEC IL-11 | 27.0 |
| Secondary Tr1 rest | 12.8 | Lung Microvascular EC none | 24.5 |
| Primary Th1 act | 9.4 | Lung Microvascular EC TNF | 24.3 |
| | | alpha + IL-1beta | |
| Primary Th2 act | 9.5 | Microvascular Dermal EC none | 46.3 |
| Primary Tr1 act | 12.5 | Microsvasular Dermal EC | 22.2 |
| | | TNF alpha + IL-1beta | |
| Primary Th1 rest | 56.3 | Bronchial epithelium TNF alpha + | 27.5 |
| | | IL1beta | |
| Primary Th2 rest | 26.1 | Small airway epithelium none | 9.6 |
| Primary Tr1 rest | 19.2 | Small airway epithelium TNF | 50.0 |
| | | alpha + IL-1beta | |
| CD45RA CD4 lymphocyte | 9.9 | Coronery artery SMC rest | 13.0 |
| act | | | |
| CD45RO CD4 lymphocyte | 24.0 | Coronery artery SMC TNF alpha + | 6.6 |
| act | | IL-1beta | |
| CD8 lymphocyte act | 11.9 | Astrocytes rest | 6.2 |
| Secondary CD8 lymphocyte | 13.8 | Astrocytes TNF alpha + IL-1beta | 5.0 |
| rest | | | |
| Secondary CD8 lymphocyte | 10.5 | KU-812 (Basophil) rest | 28.7 |
| act | | | |
| CD4 lymphocyte none | 9.1 | KU-812 (Basophil) | 100.0 |
| | | PMA/ionomycin | |
| 2ry Th1/Th2/Tr1_anti-CD95 | 22.8 | CCD1106 (Keratinocytes) none | 24.5 |
| CH11 | | | |
| LAK cells rest | 1.6 | CCD1106 (Keratinocytes) | 12.9 |
| | | TNF alpha + IL-1beta | |
| LAK cells IL-2 | 21.0 | Liver cirrhosis | 3.4 |
| LAK cells IL-2 + IL-12 | 17.3 | Lupus kidney | 4.1 |
| LAK cells IL-2 + IFN gamma | 28.5 | NCI-H292 none | 31.4 |
| LAK cells IL-2 + IL-18 | 27.9 | NCI-H292 IL-4 | 22.7 |
| LAK cells PMA/ionomycin | 7.1 | NCI-H292 IL-9 | 28.5 |
| NK Cells IL-2 rest | 16.5 | NCI-H292 IL-13 | 11.2 |
| Two Way MLR 3 day | 18.8 | NCI-H292 IFN gamma | 15.1 |
| Two Way MLR 5 day | 9.7 | HPAEC none | 24.1 |
| Two Way MLR 7 day | 9.3 | HPAEC TNF alpha + IL-1beta | 17.8 |
| PBMC rest | 8.2 | Lung fibroblast none | 7.1 |
| PBMC PWM | 52.9 | Lung fibroblast TNF alpha + IL- | 10.3 |
| | | 1beta | |
| PBMC PHA-L | 24.8 | Lung fibroblast IL-4 | 10.3 |
| Ramos (B cell) none | 24.1 | Lung fibroblast IL-9 | 12.5 |
| Ramos (B cell) ionomycin | 81.8 | Lung fibroblast IL-13 | 8.3 |
| B lymphocytes PWM | 49.0 | Lung fibroblast IFN gamma | 16.8 |
| B lymphocytes CD40L and | 27.0 | Dermal fibroblast CCD1070 rest | 22.8 |
| IL-4 | | | |
| EOL-1 dbcAMP | 15.4 | Dermal fibroblast CCD1070 TNF | 45.4 |
| | | alpha | |
| EOL-1 dbcAMP | 24.5 | Dermal fibroblast CCD1070 IL- | 12.6 |
| PMA/ionomycin | | 1beta | |
| Dendritic cells none | 18.6 | Dermal fibroblast IFN gamma | 9.3 |
| Dendritic cells LPS | 10.4 | Dermal fibroblast IL-4 | 16.6 |
| Dendritic cells anti-CD40 | 14.1 | IBD Colitis 2 | 2.6 |
| Monocytes rest | 11.0 | IBD Crohn's | 3.5 |
| Monocytes LPS | 7.3 | Colon | 13.9 |
| Macrophages rest | 19.3 | Lung | 11.8 |
| Macrophages LPS | 11.7 | Thymus | 29.1 |
| HUVEC none | 30.6 | Kidney | 41.8 |
| HUVEC starved | 63.7 | | |

CNS_neurodegeneration_v1.0 Summary: Ag3259 This panel confirms the expression of the CG57505-01 gene at moderate levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3259 Expression of the CG57505-01 gene is highest in a gastric cancer cell line (CT=25.6). Interestingly, expression of this gene appears to be higher in cancer cell lines than in normal tissues. Specifically, CG57505-01 gene expression appears to be upregulated in colon, gastric, renal, lung and breast cancer cell lines. Therefore, therapeutic modulation of the activity of this gene or its protein product, through the use of small molecule drugs, protein therapeutics or antibodies, might be beneficial in the treatment of these cancers.

In addition, this gene is expressed at moderate levels in all regions of the central nervous system examined, including in amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at moderate levels in adipose, adrenal gland, thyroid, pituitary gland, gastrointestinal tract, pancreas, skeletal muscle, heart and liver. Therefore, therapeutic modulation of the activity of this gene may prove useful in the treatment of endocrine/metabolically related diseases, such as obesity and diabetes. This gene is moderately expression in pancreas, which includes the insulin-secreting islets of Langerhans. Insulin secretion is augmented by protein kinase C activation. The CG57505-01 gene encodes a protein with homology to a protein kinase C-binding protein, and as such, may be a therapeutic modulator of insulin secretion in Type 2 diabetes.

Panel 4D Summary: Ag3259 The CG57505-01 transcript is expressed in the PMA and ionomycin treated basophil cell line KU-812 (CT=27) and to a lesser extent in untreated KU-812 cells (CT=29). Therefore, antibody or small molecule therapies designed with the protein encoded for by this gene could block or inhibit inflammation or tissue damage due to basophil activation in response to asthma, allergies, hypersensitivity reactions, psoriasis, and viral infections.

This gene is also expressed at low levels in Ramos (B cells) (CT=29) and expression of this gene is stimulated in these cells by ionomycin (CT=27). Expression of this gene in B cells suggests that this gene may be involved in rheumatic disease including rheumatoid arthritis, lupus, osteoarthritis, and hyperproliferative B cell disorders.

Furthermore, treatment of small airway epithelium with TNFalpha+IL-1 beta stimulated the expression of the CG57721-01 gene. Therefore, modulation of the expression or activity of the protein encoded by this transcript through the application of small molecule therapeutics may be useful in the treatment of asthma, COPD, and emphysema.

NOV62

Expression of NOV62/CG57473-01 was assessed using the primer-probe set Ag3251, described in Table AZA. Please note that CG57473-02 represents a full-length physical clone of the CG57473-01 gene, validating the prediction of the gene sequence.

TABLE AZA
Probe Name Ag3251

| Primers | Sequences | Length | Start Position | SEQ ID NO |
|---|---|---|---|---|
| Forward | 5′-tctcaggaagtggtgaagga-3′ | 20 | 993 | 437 |
| Probe | TET-5′-caattctccaaggatacattggaaaa-3′-TAMRA | 26 | 1020 | 438 |
| Reverse | 5′-atacaaaccctcggaacga-3′ | 19 | 1058 | 439 |

CNS_neurodegeneration_v1.0 Summary: Ag3251 Expression of this gene is low/undetectable (CTs>35) across all of the samples on this panel (data not shown).

General_screening_panel_v1.4 Summary: Ag3251 Results from one experiment with the CG57473-01 gene are not included. The amp plot indicates that there were experimental difficulties with this run.

Panel 4D Summary: Ag3251 Expression of this gene is low/undetectable (CTs>35) across all of the samples on this panel (data not shown).

NOV63

Expression of NOV63/CG57777-01 was assessed using the primer-probe set Ag3327, described in Table BFA. Results of the RTQ-PCR runs are shown in Tables BFB, BFC and BFD.

TABLE BFA
Probe Name Ag3327

| Primers | Sequences | Length | Start Position | SEQ ID NO |
|---|---|---|---|---|
| Forward | 5′-ttctgtgaaacaatgagaaca-3′ | 22 | 424 | 440 |
| Probe | TET-5′-cacaacgtaccagaatttctgggaca-3′-TAMRA | 26 | 450 | 441 |
| Reverse | 5′-ccctctaaccactgctttagct-3′ | 22 | 477 | 442 |

TABLE BFB
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3327, Run | Ag3327, Run | | Ag3327, Run | Ag3327, Run |
| Tissue Name | 210138310 | 224063127 | Tissue Name | 210138310 | 224063127 |
| AD 1 Hippo | 6.1 | 6.0 | Control (Path) 3 | 14.0 | 12.3 |
| | | | Temporal Ctx | | |
| AD 2 Hippo | 28.1 | 29.3 | Control (Path) 4 | 69.7 | 63.7 |
| | | | Temporal Ctx | | |
| AD 3 Hippo | 14.9 | 13.5 | AD 1 Occipital | 18.8 | 31.4 |
| | | | Ctx | | |
| AD 4 Hippo | 24.7 | 15.9 | AD 2 Occipital | 0.0 | 0.0 |
| | | | Ctx (Missing) | | |
| AD 5 Hippo | 59.0 | 65.1 | AD 3 Occipital | 17.1 | 9.1 |
| | | | Ctx | | |
| AD 6 Hippo | 45.7 | 52.5 | AD 4 Occipital | 48.0 | 35.8 |
| | | | Ctx | | |
| Control 2 Hippo | 38.2 | 28.7 | AD 5 Occipital | 52.9 | 0.0 |
| | | | Ctx | | |
| Control 4 Hippo | 15.5 | 16.4 | AD 6 Occipital | 23.0 | 40.1 |
| | | | Ctx | | |
| Control (Path) 3 | 11.7 | 2.5 | Control 1 | 1.2 | 2.1 |
| Hippo | | | Occipital Ctx | | |
| AD 1 Temporal | 16.3 | 14.2 | Control 2 | 31.6 | 59.0 |
| Ctx | | | Occipital Ctx | | |
| AD 2 Temporal | 21.9 | 29.5 | Control 3 | 62.9 | 60.3 |
| Ctx | | | Occipital Ctx | | |
| AD 3 Temporal | 15.1 | 12.2 | Control 4 | 10.2 | 6.9 |
| Ctx | | | Occipital Ctx | | |
| AD 4 Temporal | 24.7 | 16.0 | Control (Path) 1 | 100.0 | 96.6 |
| Ctx | | | Occipital Ctx | | |
| AD 5 Inf | 85.9 | 41.8 | Control (Path) 2 | 44.1 | 10.0 |
| Temporal Ctx | | | Occipital Ctx | | |
| AD 5 Sup | 47.3 | 59.9 | Control (Path) 3 | 1.7 | 2.0 |
| Temporal Ctx | | | Occipital Ctx | | |
| AD 6 Inf | 57.4 | 61.6 | Control (Path) 4 | 55.5 | 41.2 |
| Temporal Ctx | | | Occipital Ctx | | |
| AD 6 Sup | 77.9 | 70.7 | Control 1 | 9.0 | 7.1 |
| Temporal Ctx | | | Parietal Ctx | | |
| Control 1 | 14.3 | 13.6 | Control 2 | 52.9 | 67.8 |
| Temporal Ctx | | | Parietal Ctx | | |
| Control 2 | 34.9 | 18.8 | Control 3 | 15.4 | 10.9 |
| Temporal Ctx | | | Parietal Ctx | | |
| Control 3 | 42.6 | 28.1 | Control (Path) 1 | 84.1 | 77.4 |
| Temporal Ctx | | | Parietal Ctx | | |
| Control 3 | 19.6 | 13.1 | Control (Path) 2 | 43.8 | 58.6 |
| Temporal Ctx | | | Parietal Ctx | | |
| Control (Path) 1 | 75.8 | 100.0 | Control (Path) 3 | 9.2 | 9.0 |
| Temporal Ctx | | | Parietal Ctx | | |
| Control (Path) 2 | 98.6 | 72.7 | Control (Path) 4 | 89.5 | 75.8 |
| Temporal Ctx | | | Parietal Ctx | | |

TABLE BFC
General_screening_panel_v1.4

| | Rel. Exp. (%) | | Rel. Exp. (%) |
|---|---|---|---|
| | Ag3327, Run | | Ag3327, Run |
| Tissue Name | 215678614 | Tissue Name | 215678614 |
| Adipose | 2.3 | Renal ca. TK-10 | 13.3 |
| Melanoma* Hs688(A).T | 2.8 | Bladder | 17.1 |
| Melanoma* Hs688(B).T | 1.1 | Gastric ca. (liver met.) NCI- | 0.0 |
| | | N87 | |
| Melanoma* M14 | 0.0 | Gastric ca. KATO III | 1.2 |
| Melanoma* LOXIMVI | 0.7 | Colon ca. SW-948 | 0.0 |
| Melanoma* SK-MEL-5 | 0.6 | Colon ca. SW480 | 8.1 |
| Squamous cell carcinoma | 2.0 | Colon ca.* (SW480 met) | 0.0 |
| SCC-4 | | SW620 | |
| Testis Pool | 15.1 | Colon ca. HT29 | 1.5 |
| Prostate ca.* (bone met) | 0.6 | Colon ca. HCT-116 | 8.2 |
| PC-3 | | | |
| Prostate Pool | 1.6 | Colon ca. CaCo-2 | 6.1 |
| Placenta | 0.0 | Colon cancer tissue | 0.0 |
| Uterus Pool | 0.0 | Colon ca. SW1116 | 0.0 |
| Ovarian ca. OVCAR-3 | 1.6 | Colon ca. Colo-205 | 0.0 |
| Ovarian ca. SK-OV-3 | 1.3 | Colon ca. SW-48 | 0.0 |
| Ovarian ca. OVCAR-4 | 1.5 | Colon Pool | 0.0 |
| Ovarian ca. OVCAR-5 | 7.8 | Small Intestine Pool | 0.7 |
| Ovarian ca. IGROV-1 | 0.0 | Stomach Pool | 4.1 |
| Ovarian ca. OVCAR-8 | 1.1 | Bone Marrow Pool | 1.0 |
| Ovary | 4.6 | Fetal Heart | 0.0 |
| Breast ca. MCF-7 | 0.0 | Heart Pool | 0.0 |
| Breast ca. MDA-MB-231 | 0.0 | Lymph Node Pool | 0.6 |
| Breast ca. BT 549 | 9.2 | Fetal Skeletal Muscle | 0.0 |
| Breast ca. T47D | 1.5 | Skeletal Muscle Pool | 3.5 |
| Breast ca. MDA-N | 0.0 | Spleen Pool | 1.4 |
| Breast Pool | 14.4 | Thymus Pool | 2.2 |
| Trachea | 0.0 | CNS cancer (glio/astro) U87- | 13.2 |
| | | MG | |
| Lung | 16.7 | CNS cancer (glio/astro) U- | 0.0 |
| | | 118-MG | |
| Fetal Lung | 15.6 | CNS cancer (neuro; met) SK- | 1.3 |
| | | N-AS | |
| Lung ca. NCI-N417 | 1.8 | CNS cancer (astro) SF-539 | 1.3 |
| Lung ca. LX-1 | 2.0 | CNS cancer (astro) SNB-75 | 100.0 |
| Lung ca. NCI-H146 | 1.6 | CNS cancer (glio) SNB-19 | 2.7 |
| Lung ca. SHP-77 | 0.0 | CNS cancer (glio) SF-295 | 39.8 |
| Lung ca. A549 | 12.7 | Brain (Amygdala) Pool | 4.3 |
| Lung ca. NCI-11526 | 1.4 | Brain (cerebellum) | 8.7 |
| Lung ca. NCI-H23 | 3.1 | Brain (fetal) | 0.6 |
| Lung ca. NCI-H460 | 8.4 | Brain (Hippocampus) Pool | 3.0 |
| Lung ca. HOP-62 | 0.9 | Cerebral Cortex Pool | 12.0 |
| Lung ca. NCI-H522 | 1.2 | Brain (Substantia nigra) Pool | 3.9 |
| Liver | 0.0 | Brain (Thalamus) Pool | 25.9 |
| Fetal Liver | 0.0 | Brain (whole) | 8.8 |
| Liver ca. HepG2 | 0.8 | Spinal Cord Pool | 0.8 |
| Kidney Pool | 0.0 | Adrenal Gland | 0.0 |
| Fetal Kidney | 0.7 | Pituitary gland Pool | 1.3 |
| Renal ca. 786-0 | 21.5 | Salivary Gland | 0.0 |
| Renal ca. A498 | 11.8 | Thyroid (female) | 3.1 |
| Renal ca. ACHN | 1.1 | Pancreatic ca. CAPAN2 | 1.2 |
| Renal ca. UO-31 | 2.7 | Pancreas Pool | 6.3 |

TABLE BFD
Panel 4D

| | Rel. Exp. (%) | | Rel. Exp. (%) |
|---|---|---|---|
| | Ag3327, Run | | Ag3327, Run |
| Tissue Name | 165021004 | Tissue Name | 165021004 |
| Secondary Th1 act | 11.3 | HUVEC IL-1beta | 4.0 |
| Secondary Th2 act | 12.5 | HUVEC IFN gamma | 12.3 |
| Secondary Tr1 act | 17.9 | HUVEC TNF alpha + IFN gamma | 2.3 |
| Secondary Th1 rest | 4.4 | HUVEC TNF alpha + IL4 | 1.8 |
| Secondary Th2 rest | 6.0 | HUVEC IL-11 | 6.5 |
| Secondary Tr1 rest | 6.4 | Lung Microvascular EC none | 8.5 |
| Primary Th1 act | 20.6 | Lung Microvascular EC | 31.6 |
| | | TNF alpha + IL-1beta | |
| Primary Th2 act | 13.8 | Microvascular Dermal EC none | 17.9 |
| Primary Tr1 act | 17.1 | Microsvasular Dermal EC | 29.7 |
| | | TNF alpha + IL-1beta | |
| Primary Th1 rest | 25.5 | Bronchial epithelium TNF alpha + | 4.2 |
| | | IL1beta | |
| Primary Th2 rest | 11.7 | Small airway epithelium none | 2.0 |
| Primary Tr1 rest | 4.6 | Small airway epithelium | 13.2 |
| | | TNF alpha + IL1beta | |
| CD45RA CD4 lymphocyte | 9.3 | Coronery artery SMC rest | 9.2 |
| act | | | |
| CD45RO CD4 lymphocyte | 12.9 | Coronery artery SMC TNF alpha + | 2.4 |
| act | | IL-1beta | |
| CD8 lymphocyte act | 14.9 | Astrocytes rest | 12.5 |
| Secondary CD8 lymphocyte | 11.1 | Astrocytes TNF alpha + IL-1beta | 9.9 |
| rest | | | |
| Secondary CD8 lymphocyte | 13.2 | KU-812 (Basophil) rest | 7.1 |
| act | | | |
| CD4 lymphocyte none | 5.8 | KU-812 (Basophil) | 21.5 |
| | | PMA/ionomycin | |
| 2ry Th1/Th2/Tr1_anti-CD95 | 6.3 | CCD1106 (Keratinocytes) none | 7.1 |
| CH11 | | | |
| LAK cells rest | 12.8 | CCD1106 (Keratinocytes) | 2.2 |
| | | TNF alpha + IL-1beta | |
| LAK cells IL-2 | 0.0 | Liver cirrhosis | 17.7 |
| LAK cells IL-2 + IL-12 | 11.1 | Lupus kidney | 19.3 |
| LAK cells IL-2 + IFN gamma | 12.2 | NCI-H292 none | 27.7 |
| LAK cells IL-2 + IL-18 | 6.3 | NCI-H292 IL-4 | 14.9 |
| LAK cells PMA/ionomycin | 8.2 | NCI-H292 IL-9 | 14.8 |
| NK Cells IL-2 rest | 9.4 | NCI-H292 IL-13 | 2.7 |
| Two Way MLR 3 day | 8.3 | NCI-H292 IFN gamma | 3.0 |
| Two Way MLR 5 day | 4.0 | HPAEC none | 1.4 |
| Two Way MLR 7 day | 11.6 | HPAEC TNF alpha + IL-1beta | 1.2 |
| PBMC rest | 5.0 | Lung fibroblast none | 7.6 |
| PBMC PWM | 73.2 | Lung fibroblast TNF alpha + IL- | 9.0 |
| | | 1beta | |
| PBMC PHA-L | 16.2 | Lung fibroblast IL-4 | 9.5 |
| Ramos (B cell) none | 22.5 | Lung fibroblast IL-9 | 10.7 |
| Ramos (B cell) ionomycin | 100.0 | Lung fibroblast IL-13 | 8.4 |
| B lymphocytes PWM | 23.8 | Lung fibroblast IFN gamma | 9.5 |
| B lymphocytes CD40L and | 11.0 | Dermal fibroblast CCD1070 rest | 17.3 |
| IL-4 | | | |
| EOL-1 dbcAMP | 2.1 | Dermal fibroblast CCD1070 TNF | 35.8 |
| | | alpha | |
| EOL-1 dbcAMP | 2.1 | Dermal fibroblast CCD1070 IL- | 14.0 |
| PMA/ionomycin | | 1beta | |
| Dendritic cells none | 2.9 | Dermal fibroblast IFN gamma | 5.1 |
| Dendritic cells LPS | 6.3 | Dermal fibroblast IL-4 | 8.1 |
| Dendritic cells anti-CD40 | 3.7 | IBD Colitis 2 | 5.3 |
| Monocytes rest | 12.6 | IBD Crohn's | 4.5 |
| Monocytes LPS | 13.5 | Colon | 9.3 |
| Macrophages rest | 5.1 | Lung | 7.7 |
| Macrophages LPS | 9.2 | Thymus | 62.4 |
| HUVEC none | 3.8 | Kidney | 7.4 |
| HUVEC starved | 4.9 | | |

CNS_neurodegeneration_v1.0 Summary: Ag3327 This panel confirms the expression of the CG57777-01 gene at low levels in the brain in an independent group of individuals. This gene is found to be down-regulated in the temporal cortex of Alzheimer's disease patients. Therefore, up-regulation of this gene or its protein product, or treatment with specific agonists for this receptor may be of use in reversing the dementia/memory loss associated with this disease and neuronal death.

General_screening_panel_v1.4 Summary: Ag3327 The CG57777-01 codes for a homologue of LINE-1 reverse transcriptase. This gene is moderately expressed in samples derived from two of the CNS cancer cell line. Thus, the expression of this gene could be used to distinguish these samples from the other samples in the panel.

Panel 4D Summary: Ag3327 The CG57777-01 gene is expressed at low to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues.

This pattern is in agreement with the expression profile in General_screening_panel_v1.5 and also suggests a role for the gene product in cell survival and proliferation.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

NOV64

Expression of NOV64/CG57779-01 was assessed using the primer-probe set Ag3328, described in Table BGA. Results of the RTQ-PCR runs are shown in Tables BGB, BGC and BGD.

TABLE BGA
Probe Name Ag3328

| Primers | Sequences | Length | Start Position | SEQ ID NO |
|---|---|---|---|---|
| Forward | 5′-gatgaagctggaaaccatcat-3′ | 21 | 2994 | 443 |
| Probe | TET-5′-tcacaaggacagaaaaccaaacactg-3′-TAMRA | 26 | 3028 | 444 |
| Reverse | 5′-cccacctatgagtgagaacatg-3′ | 22 | 3054 | 445 |

TABLE BGB
CNS_neurodegeneration_v1.0

| | Rel. Exp. (%) | | Rel. Exp. (%) |
|---|---|---|---|
| | Ag3328, Run | | Ag3328, Run |
| Tissue Name | 210138311 | Tissue Name | 210138311 |
| AD 1 Hippo | 9.4 | Control (Path) 3 Temporal | 5.8 |
| AD 2 Hippo | 22.7 | Control (Path) 4 Temporal | 30.8 |
| | | Ctx | |
| AD 3 Hippo | 6.6 | AD 1 Occipital Ctx | 19.1 |
| AD 4 Hippo | 7.8 | AD 2 Occipital Ctx | 0.5 |
| | | (Missing) | |
| AD 5 hippo | 91.4 | AD 3 Occipital Ctx | 6.1 |
| AD 6 Hippo | 50.3 | AD 4 Occipital Ctx | 14.4 |
| Control 2 Hippo | 17.7 | AD 5 Occipital Ctx | 3.6 |
| Control 4 Hippo | 9.7 | AD 6 Occipital Ctx | 22.5 |
| Control (Path) 3 Hippo | 5.5 | Control 1 Occipital Ctx | 3.6 |
| AD 1 Temporal Ctx | 15.4 | Control 2 Occipital Ctx | 27.2 |
| AD 2 Temporal Ctx | 22.8 | Control 3 Occipital Ctx | 21.2 |
| AD 3 Temporal Ctx | 5.6 | Control 4 Occipital Ctx | 6.9 |
| AD 4 Temporal Ctx | 14.7 | Control (Path) 1 Occipital | 76.8 |
| | | Ctx | |
| AD 5 Inf Temporal Ctx | 100.0 | Control (Path) 2 Occipital | 9.5 |
| | | Ctx | |
| AD 5 SupTemporal Ctx | 68.8 | Control (Path) 3 Occipital | 2.6 |
| | | Ctx | |
| AD 6 Inf Temporal Ctx | 47.6 | Control (Path) 4 Occipital | 22.2 |
| | | Ctx | |
| AD 6 Sup Temporal Ctx | 55.9 | Control 1 Parietal Ctx | 5.0 |
| Control 1 Temporal Ctx | 5.4 | Control 2 Parietal Ctx | 53.2 |
| Control 2 Temporal Ctx | 11.7 | Control 3 Parietal Ctx | 12.9 |
| Control 3 Temporal Ctx | 10.1 | Control (Path) 1 Parietal | 49.7 |
| | | Ctx | |
| Control 4 Temporal Ctx | 7.3 | Control (Path) 2 Parietal | 22.5 |
| | | Ctx | |
| Control (Path) 1 Temporal | 42.9 | Control (Path) 3 Parietal | 7.4 |
| Ctx | | Ctx | |
| Control (Path) 2 Temporal | 34.6 | Control (Path) 4 Parietal | 46.3 |
| Ctx | | Ctx | |

TABLE BGC
General_screening_panel_v1.4

| | Rel. Exp. (%) Ag3328, Run | | Rel. Exp. (%) Ag3328, Run |
|---|---|---|---|
| Tissue Name | 215678615 | Tissue Name | 215678615 |
| Adipose | 20.0 | Renal ca. TK-10 | 27.2 |
| Melanoma* Hs688(A).T | 18.9 | Bladder | 39.8 |
| Melanoma* Hs688(B).T | 14.0 | Gastric ca. (liver met.) NCI- | 48.0 |
| | | N87 | |
| Melanoma* M14 | 11.6 | Gastric ca. KATO III | 25.5 |
| Melanoma* LOXIMVI | 13.3 | Colon ca. SW-948 | 3.6 |
| Melanoma* SK-MEL-5 | 28.1 | Colon ca. SW480 | 33.9 |
| Squamous cell carcinoma | 11.0 | Colon ca.* (SW480 met) | 24.1 |
| SCC-4 | | SW620 | |
| Testis Pool | 15.7 | Colon ca. HT29 | 12.7 |
| Prostate ca.* (bone met) | 24.1 | Colon ca. HCT-116 | 24.5 |
| PC-3 | | | |
| Prostate Pool | 12.3 | Colon ca. CaCo-2 | 28.7 |
| Placenta | 6.1 | Colon cancer tissue | 20.7 |
| Uterus Pool | 7.3 | Colon ca. SW1116 | 6.7 |
| Ovarian ca. OVCAR-3 | 33.7 | Colon ca. Colo-205 | 3.7 |
| Ovarian ca. SK-OV-3 | 69.3 | Colon ca. SW-48 | 3.5 |
| Ovarian ca. OVCAR-4 | 10.4 | Colon Pool | 41.2 |
| Ovarian ca. OVCAR-5 | 54.0 | Small Intestine Pool | 51.4 |
| Ovarian ca. IGROV-1 | 16.4 | Stomach Pool | 5.2 |
| Ovarian ca. OVCAR-8 | 11.3 | Bone Marrow Pool | 22.7 |
| Ovary | 15.1 | Fetal Heart | 34.2 |
| Breast ca. MCF-7 | 25.3 | Heart Pool | 16.2 |
| Breast ca. MDA-MB-231 | 29.1 | Lymph Node Pool | 44.1 |
| Breast ca. BT 549 | 33.0 | Fetal Skeletal Muscle | 26.1 |
| Breast ca. T47D | 44.4 | Skeletal Muscle Pool | 7.7 |
| Breast ca. MDA-N | 17.7 | Spleen Pool | 21.5 |
| Breast Pool | 2.4 | Thymus Pool | 33.2 |
| Trachea | 26.1 | CNS cancer (glio/astro) U87- | 23.5 |
| | | MG | |
| Lung | 27.0 | CNS cancer (glio/astro) U- | 38.4 |
| | | 118-MG | |
| Fetal Lung | 100.0 | CNS cancer (neuro; met) SK- | 27.7 |
| | | N-AS | |
| Lung ca. NCI-N417 | 1.4 | CNS cancer (astro) SF-539 | 12.5 |
| Lung ca. LX-1 | 35.4 | CNS cancer (astro) SNB-75 | 59.9 |
| Lung ca. NCI-H146 | 8.8 | CNS cancer (glio) SNB-19 | 14.7 |
| Lung ca. SHP-77 | 27.4 | CNS cancer (glio) SF-295 | 64.2 |
| Lung ca. A549 | 21.8 | Brain (Amygdala) Pool | 13.4 |
| Lung ca. NCI-H526 | 5.6 | Brain (cerebellum) | 14.3 |
| Lung ca. NCI-H23 | 39.5 | Brain (fetal) | 28.9 |
| Lung ca. NCI-H460 | 31.6 | Brain (Hippocampus) Pool | 14.6 |
| Lung ca. HOP-62 | 23.2 | Cerebral Cortex Pool | 21.3 |
| Lung ca. NCI-H522 | 42.3 | Brain (Substantia nigra) Pool | 14.2 |
| Liver | 0.5 | Brain (Thalamus) Pool | 32.8 |
| Fetal Liver | 13.6 | Brain (whole) | 12.8 |
| Liver ca. HepG2 | 11.2 | Spinal Cord Pool | 17.4 |
| Kidney Pool | 49.3 | Adrenal Gland | 24.1 |
| Fetal Kidney | 54.3 | Pituitary gland Pool | 7.9 |
| Renal ca. 786-0 | 31.9 | Salivary Gland | 5.5 |
| Renal ca. A498 | 5.4 | Thyroid (female) | 3.7 |
| Renal ca. ACHN | 15.1 | Pancreatic ca. CAPAN2 | 33.0 |
| Renal ca. UO-31 | 21.8 | Pancreas Pool | 58.2 |

TABLE BGD
Panel 4D

| | Rel. Exp. (%) Ag3328, Run | | Rel. Exp. (%) Ag3328, Run |
|---|---|---|---|
| Tissue Name | 165021014 | Tissue Name | 165021014 |
| Secondary Th1 act | 21.0 | HUVEC IL-1 beta | 4.7 |
| Secondary Th2 act | 26.4 | HUVEC IFN gamma | 15.0 |
| Secondary Tr1 act | 39.8 | HUVEC TNF alpha + IFN gamma | 13.4 |
| Secondary Th1 rest | 2.8 | HUVEC TNF alpha + IL4 | 0.0 |
| Secondary Th2 rest | 12.6 | HUVEC IL-11 | 12.2 |
| Secondary Tr1 rest | 15.5 | Lung Microvascular EC none | 16.5 |
| Primary Th1 act | 29.1 | Lung Microvascular EC TNF alpha + | 18.6 |
| | | IL-1 beta | |
| Primary Th2 act | 25.9 | Microvascular Dermal EC none | 16.2 |
| Primary Tr1 act | 22.8 | Microsvasular Dermal EC | 14.4 |
| | | TNF alpha + IL-1 beta | |
| Primary Th1 rest | 36.9 | Bronchial epithelium TNF alpha + | 12.6 |
| | | IL1 beta | |
| Primary Th2 rest | 18.8 | Small airway epithelium none | 4.2 |
| Primary Tr1 rest | 15.3 | Small airway epithelium TNF alpha + | 30.1 |
| | | IL-1 beta | |
| CD45RA CD4 lymphocyte | 11.8 | Coronery artery SMC rest | 6.5 |
| act | | | |
| CD45RO CD4 lymphocyte | 25.0 | Coronery artery SMC TNF alpha + | 3.0 |
| act | | IL-1 beta | |
| CD8 lymphocyte act | 17.0 | Astrocytes rest | 10.1 |
| Secondary CD8 lymphocyte | 19.5 | Astrocytes TNF alpha + IL-1 beta | 10.0 |
| rest | | | |
| Secondary CD8 lymphocyte | 15.9 | KU-812 (Basophil) rest | 11.0 |
| act | | | |
| CD4 lymphocyte none | 11.9 | KU-812 (Basophil) | 31.6 |
| | | PMA/ionomycin | |
| 2ry Th1/Th2/Tr1_anti-CD95 | 24.0 | CCD1106 (Keratinocytes) none | 9.8 |
| CH11 | | | |
| LAK cells rest | 21.9 | CCD1106 (Keratinocytes) | 6.7 |
| | | TNF alpha + IL-1 beta | |
| LAK cells IL-2 | 21.3 | Liver cirrhosis | 26.1 |
| LAK cells IL-2 + IL-12 | 13.5 | Lupus kidney | 7.8 |
| LAK cells IL-2 + IFN gamma | 26.8 | NCI-H292 none | 29.9 |
| LAK cells IL-2 + IL-18 | 18.9 | NCI-H292 IL-4 | 24.5 |
| LAK cells PMA/ionomycin | 11.0 | NCI-H292 IL-9 | 22.1 |
| NK Cells IL-2 rest | 18.4 | NCI-H292 IL-13 | 13.1 |
| Two Way MLR 3 day | 23.2 | NCI-H292 IFN gamma | 18.7 |
| Two Way MLR 5 day | 8.8 | HPAEC none | 8.7 |
| Two Way MLR 7 day | 10.8 | HPAEC TNF alpha + IL-1 beta | 18.7 |
| PBMC rest | 9.9 | Lung fibroblast none | 9.6 |
| PBMC PWM | 48.3 | Lung fibroblast TNF alpha + IL-1 | 6.3 |
| | | beta | |
| PBMC PHA-L | 16.7 | Lung fibroblast IL-4 | 20.3 |
| Ramos (B cell) none | 19.3 | Lung fibroblast IL-9 | 13.8 |
| Ramos (B cell) ionomycin | 43.8 | Lung fibroblast IL-13 | 14.9 |
| B lymphocytes PWM | 19.3 | Lung fibroblast IFN gamma | 15.7 |
| B lymphocytes CD40L and | 22.7 | Dermal fibroblast CCD1070 rest | 24.0 |
| IL-4 | | | |
| EOL-1 dbcAMP | 12.2 | Dermal fibroblast CCD1070 TNF | 50.7 |
| | | alpha | |
| EOL-1 dbcAMP | 15.2 | Dermal fibroblast CCD1070 IL-1 | 11.4 |
| PMA/ionomycin | | beta | |
| Dendritic cells none | 10.5 | Dermal fibroblast IFN gamma | 12.5 |
| Dendritic cells LPS | 12.5 | Dermal fibroblast IL-4 | 21.8 |
| Dendritic cells anti-CD40 | 10.8 | IBD Colitis 2 | 8.3 |
| Monocytes rest | 19.9 | IBD Crohn's | 7.2 |
| Monocytes LPS | 13.9 | Colon | 30.4 |
| Macrophages rest | 20.4 | Lung | 17.0 |
| Macrophages LPS | 7.9 | Thymus | 84.7 |
| HUVEC none | 8.2 | Kidney | 100.0 |
| HUVEC starved | 10.0 | | |

CNS_neurodegeneration_v1.0 Summary: Ag3328 This panel confirms the expression of CG57779-01 gene at low levels in the brains of an independent group of individuals. However, no differential expression of this gene was detected between Alzheimer's diseased postmortem brains and those of non-demented controls in this experiment.

General_screening_panel_v1.4 Summary: Ag3328 The CG57779-01 gene encodes a homolog of LINE-1 reverse transcriptase. Its expression is moderate to high across all of the samples on this panel. Therefore, this gene may be playing an important role in cellular function

Panel 4D Summary: Ag3328 The CG57779-01 gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues.

This pattern is in agreement with the expression profile in General_screening_panel_v1.4 and also suggests a role for the gene product in cell survival and proliferation.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

NOV65

Expression of NOV65/CG57781-01 was assessed using the primer-probe set Ag3329, described in Table BHA. Results of the RTQ-PCR runs are shown in Tables BHB, BHC and BHD.

TABLE BHA
Probe Name Ag3329

| Primers | Sequences | Length | Start Position | SEQ ID NO |
|---|---|---|---|---|
| Forward | 5′-tcacaaggacagaaaacaaac-3′ | 22 | 2944 | 446 |
| Probe | TET-5′-ccacatgttctcactcataggtggga-3′-TAMRA | 26 | 2967 | 447 |
| Reverse | 5′-gtgtccatgtgttctcgttgtt-3′ | 22 | 2997 | 448 |

TABLE BHB
CNS_neurodegeneration_v1.0

| | Rel. Exp. (%) Ag3329, Run | | Rel. Exp. (%) Ag3329, Run |
|---|---|---|---|
| Tissue Name | 210146230 | Tissue Name | 210146230 |
| AD 1 Hippo | 9.2 | Control (Path) 3 Temporal | 6.4 |
| | | Ctx | |
| AD 2 Hippo | 19.9 | Control (Path) 4 Temporal | 35.4 |
| | | Ctx | |
| AD 3 Hippo | 10.0 | AD 1 Occipital Ctx | 20.3 |
| AD 4 Hippo | 10.0 | AD 2 Occipital Ctx | 0.1 |
| | | (Missing) | |
| AD 5 Hippo | 64.6 | AD 3 Occipital Ctx | 6.4 |
| AD 6 Hippo | 42.9 | AD 4 Occipital Ctx | 18.6 |
| Control 2 Hippo | 20.4 | AD 5 Occipital Ctx | 16.0 |
| Control 4 Hippo | 10.7 | AD 6 Occipital Ctx | 18.0 |
| Control (Path) 3 Hippo | 5.5 | Control 1 Occipital Ctx | 3.1 |
| AD 1 Temporal Ctx | 20.4 | Control 2 Occipital Ctx | 28.7 |
| AD 2 Temporal Ctx | 23.7 | Control 3 Occipital Ctx | 24.1 |
| AD 3 Temporal Ctx | 8.7 | Control 4 Occipital Ctx | 6.0 |
| AD 4 Temporal Ctx | 19.9 | Control (Path) 1 Occipital | 75.3 |
| | | Ctx | |
| AD 5 Inf Temporal Ctx | 100.0 | Control (Path) 2 Occipital | 13.2 |
| | | Ctx | |
| AD 5 Sup Temporal Ctx | 65.5 | Control (Path) 3 Occipital | 3.0 |
| | | Ctx | |
| AD 6 Inf Temporal Ctx | 46.3 | Control (Path) 4 Occipital | 0.0 |
| | | Ctx | |
| AD 6 Sup Temporal Ctx | 62.0 | Control 1 Parietal Ctx | 5.4 |
| Control 1 Temporal Ctx | 5.7 | Control 2 Parietal Ctx | 43.8 |
| Control 2 Temporal Ctx | 15.7 | Control 3 Parietal Ctx | 11.8 |
| Control 3 Temporal Ctx | 16.6 | Control (Path) 1 Parietal | 47.0 |
| | | Ctx | |
| Control 3 Temporal Ctx | 10.3 | Control (Path) 2 Parietal | 23.0 |
| | | Ctx | |
| Control (Path) 1 Temporal | 50.0 | Control (Path) 3 Parietal | 5.9 |
| Ctx | | Ctx | |
| Control (Path) 2 Temporal | 44.1 | Control (Path) 4 Parietal | 39.5 |
| Ctx | | Ctx | |

TABLE BHC
General_screening_panel_v1.4

| | Rel. Exp. (%) Ag3329, Run | | Rel. Exp. (%) Ag3329, Run |
|---|---|---|---|
| Tissue Name | 215678554 | Tissue Name | 215678554 |
| Adipose | 18.4 | Renal ca. TK-10 | 31.0 |
| Melanoma* Hs688(A).T | 15.3 | Bladder | 48.0 |
| Melanoma* Hs688(B).T | 13.7 | Gastric ca. (liver met.) NCI- | 38.4 |
| | | N87 | |
| Melanoma* M14 | 21.3 | Gastric ca. KATO III | 22.2 |
| Melanoma* LOXIMVI | 15.8 | Colon ca. SW-948 | 4.3 |
| Melanoma* SK-MEL-5 | 39.0 | Colon ca. SW480 | 38.4 |
| Squamous cell carcinoma | 17.9 | Colon ca.* (SW480 met) | 21.9 |
| SCC-4 | | SW620 | |
| Testis Pool | 10.4 | Colon ca. HT29 | 15.7 |
| Prostate ca.* (bone met) | 31.0 | Colon ca. HCT-116 | 21.3 |
| PC-3 | | | |
| Prostate Pool | 18.8 | Colon ca. CaCo-2 | 27.5 |
| Placenta | 4.4 | Colon cancer tissue | 17.1 |
| Uterus Pool | 12.6 | Colon ca. SW1116 | 5.2 |
| Ovarian ca. OVCAR-3 | 22.7 | Colon ca. Colo-205 | 3.8 |
| Ovarian ca. SK-OV-3 | 34.9 | Colon ca. SW-48 | 3.3 |
| Ovarian ca. OVCAR-4 | 5.6 | Colon Pool | 79.0 |
| Ovarian ca. OVCAR-5 | 44.8 | Small Intestine Pool | 59.9 |
| Ovarian ca. IGROV-1 | 15.8 | Stomach Pool | 48.3 |
| Ovarian ca. OVCAR-8 | 11.7 | Bone Marrow Pool | 39.8 |
| Ovary | 19.2 | Fetal Heart | 67.8 |
| Breast ca. MCF-7 | 26.4 | Heart Pool | 22.8 |
| Breast ca. MDA-MB-231 | 25.0 | Lymph Node Pool | 91.4 |
| Breast ca. BT 549 | 48.3 | Fetal Skeletal Muscle | 30.1 |
| Breast ca. T47D | 49.3 | Skeletal Muscle Pool | 29.7 |
| Breast ca. MDA-N | 16.3 | Spleen Pool | 28.7 |
| Breast Pool | 76.3 | Thymus Pool | 59.9 |
| Trachea | 32.5 | CNS cancer (glio/astro) U87- | 32.5 |
| | | MG | |
| Lung | 26.2 | CNS cancer (glio/astro) U- | 40.3 |
| | | 118-MG | |
| Fetal Lung | 89.5 | CNS cancer (neuro; met) SK- | 35.4 |
| | | N-AS | |
| Lung ca. NCI-N417 | 22.7 | CNS cancer (astro) SF-539 | 11.0 |
| Lung ca. LX-1 | 28.3 | CNS cancer (astro) SNB-75 | 54.3 |
| Lung ca. NCI-H146 | 10.6 | CNS cancer (glio) SNB-19 | 9.9 |
| Lung ca. SHP-77 | 26.8 | CNS cancer (glio) SF-295 | 81.2 |
| Lung ca. A549 | 23.2 | Brain (Amygdala) Pool | 19.3 |
| Lung ca. NCI-H526 | 4.7 | Brain (cerebellum) | 21.0 |
| Lung ca. NCI-H23 | 54.3 | Brain (fetal) | 47.0 |
| Lung ca. NCI-H460 | 46.0 | Brain (Hippocampus) Pool | 20.0 |
| Lung ca. HOP-62 | 18.2 | Cerebral Cortex Pool | 21.6 |
| Lung ca. NCI-H522 | 35.4 | Brain (Substantia nigra) Pool | 14.5 |
| Liver | 0.5 | Brain (Thalamus) Pool | 35.6 |
| Fetal Liver | 17.1 | Brain (whole) | 18.4 |
| Liver ca. HepG2 | 19.6 | Spinal Cord Pool | 20.6 |
| Kidney Pool | 100.0 | Adrenal Gland | 21.0 |
| Fetal Kidney | 98.6 | Pituitary gland Pool | 11.1 |
| Renal ca. 786-0 | 27.5 | Salivary Gland | 5.4 |
| Renal ca. A498 | 9.9 | Thyroid (female) | 4.4 |
| Renal ca. ACHN | 23.3 | Pancreatic ca. CAPAN2 | 39.5 |
| Renal ca. UO-31 | 31.9 | Pancreas Pool | 56.6 |

TABLE BHD
Panel 4D

| | Rel. Exp. (%) Ag3329, Run | | Rel. Exp. (%) Ag3329, Run |
|---|---|---|---|
| Tissue Name | 165021015 | Tissue Name | 165021015 |
| Secondary Th1 act | 27.4 | HUVEC IL-1 beta | 10.4 |
| Secondary Th2 act | 0.0 | HUVEC IFN gamma | 0.0 |
| Secondary Tr1 act | 25.0 | HUVEC TNF alpha + IFN gamma | 6.6 |
| Secondary Th1 rest | 7.5 | HUVEC TNF alpha + IL4 | 5.8 |
| Secondary Th2 rest | 10.2 | HUVEC IL-11 | 5.4 |
| Secondary Tr1 rest | 12.7 | Lung Microvascular EC none | 7.9 |
| Primary Th1 act | 24.0 | Lung Microvascular EC TNF alpha + | 9.9 |
| | | IL-1 beta | |
| Primary Th2 act | 21.6 | Microvascular Dermal EC none | 9.5 |
| Primary Tr1 act | 27.9 | Microsvasular Dermal EC | 8.5 |
| | | TNF alpha + IL-1 beta | |
| Primary Th1 rest | 39.0 | Bronchial epithelium TNF alpha + | 8.1 |
| | | IL1 beta | |
| Primary Th2 rest | 17.8 | Small airway epithelium none | 2.1 |
| Primary Tr1 rest | 20.7 | Small airway epithelium TNF alpha + | 16.5 |
| | | IL-1 beta | |
| CD45RA CD4 lymphocyte | 10.7 | Coronery artery SMC rest | 5.4 |
| act | | | |
| CD45RO CD4 lymphocyte | 21.0 | Coronery artery SMC TNF alpha + | 2.7 |
| act | | IL-1 beta | |
| CD8 lymphocyte act | 11.3 | Astrocytes rest | 7.4 |
| Secondary CD8 lymphocyte | 11.8 | Astrocytes TNF alpha + IL-1 beta | 6.7 |
| rest | | | |
| Secondary CD8 lymphocyte | 9.9 | KU-812 (Basophil) rest | 9.4 |
| act | | | |
| CD4 lymphocyte none | 11.5 | KU-812 (Basophil) | 49.7 |
| | | PMA/ionomycin | |
| 2ry Th1/Th2/Tr1_anti-CD95 | 26.4 | CCD1106 (Keratinocytes) none | 6.7 |
| CH11 | | | |
| LAK cells rest | 24.5 | CCD1106 (Keratinocytes) | 4.0 |
| | | TNF alpha + IL-1 beta | |
| LAK cells IL-2 | 22.4 | Liver cirrhosis | 36.6 |
| LAK cells IL-2 + IL-12 | 10.5 | Lupus kidney | 11.3 |
| LAK cells IL-2 + IFN gamma | 14.1 | NCI-H292 none | 27.9 |
| LAK cells IL-2 + IL-18 | 15.3 | NCI-H292 IL-4 | 20.2 |
| LAK cells PMA/ionomycin | 15.4 | NCI-H292 IL-9 | 26.2 |
| NK Cells IL-2 rest | 17.7 | NCI-H292 IL-13 | 17.8 |
| Two Way MLR 3 day | 15.6 | NCI-H292 IFN gamma | 13.3 |
| Two Way MLR 5 day | 7.8 | HPAEC none | 6.5 |
| Two Way MLR 7 day | 7.5 | HPAEC TNF alpha + IL-1 beta | 12.6 |
| PBMC rest | 3.9 | Lung fibroblast none | 6.3 |
| PBMC PWM | 57.8 | Lung fibroblast TNF alpha + IL-1 | 3.8 |
| | | beta | |
| PBMC PHA-L | 19.3 | Lung fibroblast IL-4 | 11.2 |
| Ramos (B cell) none | 20.3 | Lung fibroblast IL-9 | 9.4 |
| Ramos (B cell) ionomycin | 42.9 | Lung fibroblast IL-13 | 15.9 |
| B lymphocytes PWM | 19.6 | Lung fibroblast IFN gamma | 12.9 |
| B lymphocytes CD40L and | 27.5 | Dermal fibroblast CCD1070 rest | 20.3 |
| IL-4 | | | |
| EOL-1 dbcAMP | 3.7 | Dermal fibroblast CCD1070 TNF | 73.7 |
| | | alpha | |
| EOL-1 dbcAMP | 15.0 | Dermal fibroblast CCD1070 IL-1 | 11.6 |
| PMA/ionomycin | | beta | |
| Dendritic cells none | 10.4 | Dermal fibroblast IFN gamma | 11.2 |
| Dendritic cells LPS | 10.1 | Dermal fibroblast IL-4 | 20.3 |
| Dendritic cells anti-CD40 | 10.4 | IBD Colitis 2 | 8.6 |
| Monocytes rest | 15.2 | IBD Crohn's | 5.5 |
| Monocytes LPS | 20.9 | Colon | 33.4 |
| Macrophages rest | 25.2 | Lung | 18.3 |
| Macrophages LPS | 7.1 | Thymus | 87.7 |
| HUVEC none | 5.4 | Kidney | 100.0 |
| HUVEC starved | 11.7 | | |

CNS_neurodegeneration_v1.0 Summary: Ag3329 The CG57781-01 encodes a gene that is homologous to LINE-1 reverse transcriptase. This panel confirms the expression of this gene at low levels in the brains of an independent group of individuals. However, no differential expression of this gene was detected between Alzheimer's diseased postmortem brains and those of non-demented controls in this experiment.

General_screening_panel_v1.4 Summary: Ag3329 The CG57781-01 encodes a gene that is homologous to LINE-1 reverse transcriptase. Its expression is moderate to high across all of the samples on this panel. Interestingly, this gene is expressed at much higher levels in fetal (CT=25.44) when compared to adult liver (CT=30.53). This observation suggests that expression of this gene can be used to distinguish fetal from adult liver.

Panel 4D Summary: Ag3329 This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues.

This pattern is in agreement with the expression profile in General_screening_panel_v1.4 and also suggests a role for the gene product in cell survival and proliferation.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

NOV66

Expression of NOV66/CG57783-01 was assessed using the primer-probe set Ag3330, described in Table BIA. Results of the RTQ-PCR runs are shown in Tables BIB, BIC, and BID.

TABLE BIA
Probe Name Ag3330

| Primers | Sequences | Length | Start Position | Seq id no: |
|---|---|---|---|---|
| Forward | 5′-gccatcccattactgggtatat-3′ | 22 | 2773 | 449 |
| Probe | TET-5′-tcatgctgctataaagacacatgcag-3′-TAMRA | 26 | 2812 | 450 |
| Reverse | 5′-tagtgccgcaataaacatacgt-3′ | 22 | 2838 | 451 |

TABLE BIB
CNS_neurodegeneration_v1.0

| | Rel. Exp. (%) Ag3330, Run | | Rel. Exp. (%) Ag3330, Run |
|---|---|---|---|
| Tissue Name | 210146231 | Tissue Name | 210146231 |
| AD 1 Hippo | 6.8 | Control (Path) 3 Temporal | 4.5 |
| | | Ctx | |
| AD 2 Hippo | 18.3 | Control (Path) 4 Temporal | 26.1 |
| | | Ctx | |
| AD 3 Hippo | 5.6 | AD 1 Occipital Ctx | 13.2 |
| AD 4 Hippo | 5.2 | AD 2 Occipital Ctx | 0.2 |
| | | (Missing) | |
| AD 5 hippo | 66.4 | AD 3 Occipital Ctx | 4.2 |
| AD 6 Hippo | 42.0 | AD 4 Occipital Ctx | 12.7 |
| Control 2 Hippo | 19.2 | AD 5 Occipital Ctx | 17.1 |
| Control 4 Hippo | 7.2 | AD 6 Occipital Ctx | 20.9 |
| Control (Path) 3 Hippo | 4.0 | Control 1 Occipital Ctx | 1.8 |
| AD 1 Temporal Ctx | 14.2 | Control 2 Occipital Ctx | 29.5 |
| AD 2 Temporal Ctx | 23.0 | Control 3 Occipital Ctx | 12.7 |
| AD 3 Temporal Ctx | 5.1 | Control 4 Occipital Ctx | 4.6 |
| AD 4 Temporal Ctx | 17.2 | Control (Path) 1 Occipital | 70.7 |
| | | Ctx | |
| AD 5 Inf Temporal Ctx | 100.0 | Control (Path) 2 Occipital | 11.7 |
| | | Ctx | |
| AD 5 SupTemporal Ctx | 50.7 | Control (Path) 3 Occipital | 2.0 |
| | | Ctx | |
| AD 6 Inf Temporal Ctx | 51.1 | Control (Path) 4 Occipital | 13.1 |
| | | Ctx | |
| AD 6 Sup Temporal Ctx | 52.9 | Control 1 Parietal Ctx | 5.8 |
| Control 1 Temporal Ctx | 4.2 | Control 2 Parietal Ctx | 49.3 |
| Control 2 Temporal Ctx | 13.4 | Control 3 Parietal Ctx | 11.8 |
| Control 3 Temporal Ctx | 10.2 | Control (Path) 1 Parietal | 56.6 |
| | | Ctx | |
| Control 4 Temporal Ctx | 5.7 | Control (Path) 2 Parietal | 18.9 |
| | | Ctx | |
| Control (Path) 1 Temporal | 44.1 | Control (Path) 3 Parietal | 3.1 |
| Ctx | | Ctx | |
| Control (Path) 2 Temporal | 41.2 | Control (Path) 4 Parietal | 34.9 |
| Ctx | | Ctx | |

TABLE BIC
General_screening_panel_v1.4

| | Rel. Exp. (%) Ag3330, Run | | Rel. Exp. (%) Ag3330, Run |
|---|---|---|---|
| Tissue Name | 215678685 | Tissue Name | 215678685 |
| Adipose | 27.4 | Renal ca. TK-10 | 32.8 |
| Melanoma* Hs688(A).T | 29.1 | Bladder | 54.3 |
| Melanoma* Hs688(B).T | 23.7 | Gastric ca. (liver met.) NCI- | 51.1 |
| | | N87 | |
| Melanoma* M14 | 20.7 | Gastric ca. KATO III | 31.0 |
| Melanoma* LOXIMVI | 18.2 | Colon ca. SW-948 | 5.5 |
| Melanoma* SK-MEL-5 | 44.1 | Colon ca. SW480 | 39.5 |
| Squamous cell carcinoma | 17.2 | Colon ca.* (SW480 met) | 29.7 |
| SCC-4 | | SW620 | |
| Testis Pool | 24.0 | Colon ca. HT29 | 15.3 |
| Prostate ca.* (bone met) | 41.8 | Colon ca. HCT-116 | 41.8 |
| PC-3 | | | |
| Prostate Pool | 23.0 | Colon ca. CaCo-2 | 44.1 |
| Placenta | 6.7 | Colon cancer tissue | 27.4 |
| Uterus Pool | 13.7 | Colon ca. SW1116 | 7.6 |
| Ovarian ca. OVCAR-3 | 39.0 | Colon ca. Colo-205 | 5.3 |
| Ovarian ca. SK-OV-3 | 46.3 | Colon ca. SW-48 | 3.9 |
| Ovarian ca. OVCAR-4 | 9.2 | Colon Pool | 68.3 |
| Ovarian ca. OVCAR-5 | 63.7 | Small Intestine Pool | 73.7 |
| Ovarian ca. IGROV-1 | 26.4 | Stomach Pool | 42.0 |
| Ovarian ca. OVCAR-8 | 12.4 | Bone Marrow Pool | 34.6 |
| Ovary | 24.0 | Fetal Heart | 70.2 |
| Breast ca. MCF-7 | 39.5 | Heart Pool | 33.9 |
| Breast ca. MDA-MB-231 | 33.0 | Lymph Node Pool | 64.6 |
| Breast ca. BT 549 | 49.7 | Fetal Skeletal Muscle | 31.6 |
| Breast ca. T47D | 58.2 | Skeletal Muscle Pool | 30.8 |
| Breast ca. MDA-N | 24.5 | Spleen Pool | 26.4 |
| Breast Pool | 56.3 | Thymus Pool | 44.8 |
| Trachea | 32.5 | CNS cancer (glio/astro) U87- | 44.4 |
| | | MG | |
| Lung | 43.5 | CNS cancer (glio/astro) U- | 58.2 |
| | | 118-MG | |
| Fetal Lung | 100.0 | CNS cancer (neuro; met) SK- | 45.1 |
| | | N-AS | |
| Lung ca. NCI-N417 | 11.8 | CNS cancer (astro) SF-539 | 16.7 |
| Lung ca. LX-1 | 42.6 | CNS cancer (astro) SNB-75 | 59.9 |
| Lung ca. NCI-H146 | 0.0 | CNS cancer (glio) SNB-19 | 21.0 |
| Lung ca. SHP-77 | 44.8 | CNS cancer (glio) SF-295 | 71.2 |
| Lung ca. A549 | 27.5 | Brain (Amygdala) Pool | 19.5 |
| Lung ca. NCI-H526 | 7.2 | Brain (cerebellum) | 17.2 |
| Lung ca. NCI-H23 | 57.4 | Brain (fetal) | 52.1 |
| Lung ca. NCI-H460 | 50.3 | Brain (Hippocampus) Pool | 28.3 |
| Lung ca. HOP-62 | 27.7 | Cerebral Cortex Pool | 29.5 |
| Lung ca. NCI-H522 | 51.8 | Brain (Substantia nigra) Pool | 18.3 |
| Liver | 0.9 | Brain (Thalamus) Pool | 43.5 |
| Fetal Liver | 24.8 | Brain (whole) | 23.8 |
| Liver ca. HepG2 | 17.7 | Spinal Cord Pool | 22.1 |
| Kidney Pool | 95.9 | Adrenal Gland | 23.8 |
| Fetal Kidney | 94.0 | Pituitary gland Pool | 11.6 |
| Renal ca. 786-0 | 31.6 | Salivary Gland | 7.9 |
| Renal ca. A498 | 15.5 | Thyroid (female) | 6.2 |
| Renal ca. ACHN | 28.5 | Pancreatic ca. CAPAN2 | 52.9 |
| Renal ca. UO-31 | 29.3 | Pancreas Pool | 65.1 |

TABLE BID
Panel 4D

| | Rel. Exp. (%) Ag3330, Run | | Rel. Exp. (%) Ag3330, Run |
|---|---|---|---|
| Tissue Name | 165725930 | Tissue Name | 165725930 |
| Secondary Th1 act | 20.2 | HUVEC IL-1 beta | 20.4 |
| Secondary Th2 act | 27.0 | HUVEC IFN gamma | 23.0 |
| Secondary Tr1 act | 41.8 | HUVEC TNF alpha + IFN gamma | 14.7 |
| Secondary Th1 rest | 33.4 | HUVEC TNF alpha + IL4 | 13.7 |
| Secondary Th2 rest | 15.8 | HUVEC IL-11 | 13.0 |
| Secondary Tr1 rest | 17.0 | Lung Microvascular EC none | 18.3 |
| Primary Th1 act | 14.1 | Lung Microvascular EC TNF alpha + | 13.8 |
| | | IL-1 beta | |
| Primary Th2 act | 33.2 | Microvascular Dermal EC none | 15.7 |
| Primary Tr1 act | 37.6 | Microsvasular Dermal EC | 37.9 |
| | | TNF alpha + IL-1 beta | |
| Primary Th1 rest | 100.0 | Bronchial epithelium TNF alpha + | 8.5 |
| | | IL1 beta | |
| Primary Th2 rest | 37.4 | Small airway epithelium none | 4.9 |
| Primary Tr1 rest | 34.9 | Small airway epithelium TNF alpha + | 41.5 |
| | | IL-1 beta | |
| CD45RA CD4 lymphocyte | 16.0 | Coronery artery SMC rest | 11.7 |
| act | | | |
| CD45RO CD4 lymphocyte | 28.9 | Coronery artery SMC TNF alpha + | 4.5 |
| act | | IL-1 beta | |
| CD8 lymphocyte act | 25.5 | Astrocytes rest | 33.9 |
| Secondary CD8 lymphocyte | 29.3 | Astrocytes TNF alpha + IL-1 beta | 32.1 |
| rest | | | |
| Secondary CD8 lymphocyte | 22.7 | KU-812 (Basophil) rest | 17.1 |
| act | | | |
| CD4 lymphocyte none | 21.2 | KU-812 (Basophil) | 44.8 |
| | | PMA/ionomycin | |
| 2ry Th1/Th2/Tr1_anti-CD95 | 33.2 | CCD1106 (Keratinocytes) none | 13.8 |
| CH11 | | | |
| LAK cells rest | 16.5 | CCD1106 (Keratinocytes) | 55.9 |
| | | TNF alpha + IL-1 beta | |
| LAK cells IL-2 | 48.6 | Liver cirrhosis | 76.8 |
| LAK cells IL-2 + IL-12 | 33.2 | Lupus kidney | 67.8 |
| LAK cells IL-2 + IFN gamma | 42.6 | NCI-H292 none | 39.2 |
| LAK cells IL-2 + IL-18 | 30.8 | NCI-H292 IL-4 | 57.4 |
| LAK cells PMA/ionomycin | 23.2 | NCI-H292 IL-9 | 53.2 |
| NK Cells IL-2 rest | 20.7 | NCI-H292 IL-13 | 23.2 |
| Two Way MLR 3 day | 41.8 | NCI-H292 IFN gamma | 22.8 |
| Two Way MLR 5 day | 16.0 | HPAEC none | 11.9 |
| Two Way MLR 7 day | 26.6 | HPAEC TNF alpha + IL-1 beta | 26.1 |
| PBMC rest | 16.2 | Lung fibroblast none | 19.6 |
| PBMC PWM | 33.0 | Lung fibroblast TNF alpha + IL-1 | 19.8 |
| | | beta | |
| PBMC PHA-L | 38.2 | Lung fibroblast IL-4 | 13.6 |
| Ramos (B cell) none | 42.6 | Lung fibroblast IL-9 | 15.5 |
| Ramos (B cell) ionomycin | 25.5 | Lung fibroblast IL-13 | 14.5 |
| B lymphocytes PWM | 32.1 | Lung fibroblast IFN gamma | 25.5 |
| B lymphocytes CD40L and | 36.9 | Dermal fibroblast CCD1070 rest | 32.1 |
| IL-4 | | | |
| EOL-1 dbcAMP | 20.2 | Dermal fibroblast CCD1070 TNF | 62.4 |
| | | alpha | |
| EOL-1 dbcAMP | 65.1 | Dermal fibroblast CCD1070 IL-1 | 12.0 |
| PMA/ionomycin | | beta | |
| Dendritic cells none | 17.4 | Dermal fibroblast IFN gamma | 7.2 |
| Dendritic cells LPS | 20.9 | Dermal fibroblast IL-4 | 22.7 |
| Dendritic cells anti-CD40 | 22.2 | IBD Colitis 2 | 12.0 |
| Monocytes rest | 33.0 | IBD Crohn's | 11.4 |
| Monocytes LPS | 30.1 | Colon | 95.9 |
| Macrophages rest | 19.3 | Lung | 18.3 |
| Macrophages LPS | 14.8 | Thymus | 73.2 |
| HUVEC none | 19.5 | Kidney | 67.4 |
| HUVEC starved | 35.4 | | |

AI_comprehensive panel_v1.0 Summary: Ag3330 Expression of the CG57783-01 gene is low/undetectable (CTs>35) across all of the samples on this panel (data not shown).

CNS_neurodegeneration_v1.0 Summary: Ag3330 This panel confirms the expression of the CG57783-01 gene at low levels in the brains of an independent group of individuals. However, no differential expression of this gene was detected between Alzheimer's diseased postmortem brains and those of non-demented controls in this experiment.

General_screening_panel_v1.4 Summary: Ag3330 The CG57783-01 gene encodes a protein with homology to LINE-1 reverse transcriptase. Its expression is moderate to high across all of the samples on this panel. Therefore, this gene may be playing an important role in cellular function.

Panel 4D Summary: Ag3330 The CG57783-01 gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues.

This pattern is in agreement with the expression profile in General_screening_panel_v1.4 and also suggests a role for the gene product in cell survival and proliferation.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

NOV67

Expression of NOV67A/CG57823-01 and NOV67B/CG57823-02 was assessed using the primer-probe sets Ag3514 and Ag4117, described in Tables BJA and BJB. Results of the RTQ-PCR runs are shown in Tables BJC, BJD, and BJE. Please note that Ag3514 only recognizes the CG57823-01 variant. In addition, CG57823-02 represents a full-length physical clone of the CG57823-01 gene, validating the prediction of the gene sequence.

TABLE BJA
Probe Name Ag3514

| Primers | Sequences | Length | Start Position | SEQ ID NO |
|---|---|---|---|---|
| Forward | 5′-cctcgtctcgagacaagga-3′ | 19 | 253 | 452 |
| Probe | TET-5′-accatcctcgacacactccgggag-3′-TAMRA | 24 | 274 | 453 |
| Reverse | 5′-cggtccttagtgggtttgac-3′ | 20 | 319 | 454 |

TABLE BJB
Probe Name Ag4117

| Primers | Sequences | Length | Start Position | SEQ ID NO |
|---|---|---|---|---|
| Forward | 5′-ccggtacaccaatgatctgt-3′ | 20 | 342 | 455 |
| Probe | TET-5′-cgaacatgcttctgctgcgtcct-3′-TAMRA | 23 | 383 | 456 |
| Reverse | 5′-tgacgactttccacaccaa-3′ | 19 | 406 | 457 |

TABLE BJC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3514, Run | Ag4117, Run | | Ag3514, Run | Ag4117, Run |
| Tissue Name | 210499746 | 206943850 | Tissue Name | 210499746 | 206943850 |
| AD 1 Hippo | 35.1 | 65.5 | Control (Path) 3 | 0.0 | 42.6 |
| | | | Temporal Ctx | | |
| AD 2 Hippo | 13.7 | 70.2 | Control (Path) 4 | 28.3 | 37.1 |
| | | | Temporal Ctx | | |
| AD 3 Hippo | 0.0 | 27.9 | AD 1 Occipital Ctx | 0.0 | 81.2 |
| AD 4 Hippo | 0.0 | 75.3 | AD 2 Occipital Ctx | 0.0 | 73.2 |
| | | | (Missing) | | |
| AD 5 Hippo | 80.1 | 65.1 | AD 3 Occipital Ctx | 0.0 | 71.2 |
| AD 6 Hippo | 0.0 | 67.4 | AD 4 Occipital Ctx | 75.3 | 50.0 |
| Control 2 Hippo | 0.0 | 76.8 | AD 5 Occipital Ctx | 12.9 | 66.4 |
| Control 4 Hippo | 0.0 | 65.5 | AD 6 Occipital Ctx | 0.0 | 45.4 |
| Control (Path) 3 | 0.0 | 48.3 | Control 1 Occipital Ctx | 15.1 | 74.2 |
| Hippo | | | | | |
| AD 1 Temporal | 72.2 | 49.3 | Control 2 Occipital Ctx | 0.0 | 69.3 |
| Ctx | | | | | |
| AD 2 Temporal | 0.0 | 83.5 | Control 3 Occipital Ctx | 0.0 | 38.7 |
| Ctx | | | | | |
| AD 3 Temporal | 0.0 | 46.7 | Control 4 Occipital Ctx | 41.8 | 83.5 |
| Ctx | | | | | |
| AD 4 Temporal | 0.0 | 31.6 | Control (Path) 1 | 14.0 | 72.7 |
| Ctx | | | Occipital Ctx | | |
| AD 5 Inf | 40.9 | 64.6 | Control (Path) 2 | 23.8 | 79.0 |
| Temporal Ctx | | | Occipital Ctx | | |
| AD 5 Sup | 0.0 | 23.5 | Control (Path) 3 | 0.0 | 73.2 |
| Temporal Ctx | | | Occipital Ctx | | |
| AD 6 Inf | 0.0 | 90.1 | Control (Path) 4 | 33.2 | 13.8 |
| Temporal Ctx | | | Occipital Ctx | | |
| AD 6 Sup | 0.0 | 51.4 | Control 1 Parietal Ctx | 0.0 | 39.5 |
| Temporal Ctx | | | | | |
| Control 1 | 13.8 | 63.7 | Control 2 Parietal Ctx | 0.0 | 64.2 |
| Temporal Ctx | | | | | |
| Control 2 | 0.0 | 73.7 | Control 3 Parietal Ctx | 0.0 | 51.1 |
| Temporal Ctx | | | | | |
| Control 3 | 100.0 | 29.7 | Control (Path) 1 Parietal | 50.3 | 16.8 |
| Temporal Ctx | | | Ctx | | |
| Control 3 | 0.0 | 84.7 | Control (Path) 2 Parietal | 0.0 | 25.7 |
| Temporal Ctx | | | Ctx | | |
| Control (Path) 1 | 0.0 | 51.1 | Control (Path) 3 Parietal | 0.0 | 100.0 |
| Temporal Ctx | | | Ctx | | |
| Control (Path) 2 | 26.2 | 41.2 | Control (Path) 4 Parietal | 0.0 | 37.1 |
| Temporal Ctx | | | Ctx | | |

TABLE BJD
General_screening_panel_v1.4

| | Rel. Exp. (%) | Rel. Exp. (%) | | Rel. Exp. (%) | Rel. Exp. (%) |
|---|---|---|---|---|---|
| | Ag3514, Run | Ag3514, Run | | Ag3514, Run | Ag3514, Run |
| Tissue Name | 216607683 | 222691301 | Tissue Name | 216607683 | 222691301 |
| Adipose | 0.0 | 0.0 | Renal ca. TK-10 | 0.0 | 0.0 |
| Melanoma* | 0.0 | 0.0 | Bladder | 35.6 | 0.0 |
| Hs688(A).T | | | | | |
| Melanoma* | 0.0 | 0.0 | Gastric ca. (liver | 0.0 | 0.0 |
| Hs688(B).T | | | met.) NCI-N87 | | |
| Melanoma* M14 | 0.0 | 0.0 | Gastric ca. KATO III | 0.0 | 0.0 |
| Melanoma* | 0.0 | 0.0 | Colon ca. SW-948 | 2.9 | 0.0 |
| LOXIMVI | | | | | |
| Melanoma* SK- | 0.0 | 0.0 | Colon ca. SW480 | 0.0 | 0.0 |
| MEL-5 | | | | | |
| Squamous cell | 0.0 | 0.0 | Colon ca.* (SW480 | 3.6 | 0.0 |
| carcinoma SCC-4 | | | met) SW620 | | |
| Testis Pool | 0.0 | 0.0 | Colon ca. HT29 | 0.0 | 0.0 |
| Prostate ca.* | 0.0 | 0.0 | Colon ca. HCT-116 | 0.0 | 0.0 |
| (bone met) PC-3 | | | | | |
| Prostate Pool | 0.0 | 0.0 | Colon ca. CaCo-2 | 2.8 | 0.0 |
| Placenta | 2.3 | 0.0 | Colon cancer tissue | 0.0 | 0.0 |
| Uterus Pool | 0.0 | 0.0 | Colon ca. SW1116 | 0.0 | 0.0 |
| Ovarian ca. | 0.0 | 0.0 | Colon ca. Colo-205 | 1.8 | 0.0 |
| OVCAR-3 | | | | | |
| Ovarian ca. SK- | 0.0 | 0.0 | Colon ca. SW-48 | 0.0 | 0.0 |
| OV-3 | | | | | |
| Ovarian ca. | 0.0 | 0.0 | Colon Pool | 0.0 | 0.0 |
| OVCAR-4 | | | | | |
| Ovarian ca. | 0.0 | 0.0 | Small Intestine Pool | 3.6 | 0.0 |
| OVCAR-5 | | | | | |
| Ovarian ca. | 0.0 | 0.0 | Stomach Pool | 7.2 | 0.0 |
| IGROV-1 | | | | | |
| Ovarian ca. | 0.0 | 0.0 | Bone Marrow Pool | 100.0 | 0.0 |
| OVCAR-8 | | | | | |
| Ovary | 0.0 | 0.0 | Fetal Heart | 5.7 | 0.0 |
| Breast ca. MCF-7 | 0.0 | 0.0 | Heart Pool | 0.0 | 0.0 |
| Breast ca. MDA- | 2.5 | 0.0 | Lymph Node Pool | 0.0 | 0.0 |
| MB-231 | | | | | |
| Breast ca. BT 549 | 0.0 | 0.0 | Fetal Skeletal Muscle | 21.8 | 0.0 |
| Breast ca. T47D | 0.0 | 0.0 | Skeletal Muscle Pool | 15.1 | 0.0 |
| Breast ca. MDA-N | 0.0 | 0.0 | Spleen Pool | 2.2 | 0.0 |
| Breast Pool | 0.0 | 0.0 | Thymus Pool | 0.0 | 0.0 |
| Trachea | 0.0 | 0.0 | CNS cancer | 0.0 | 0.0 |
| | | | (glio/astro) U87-MG | | |
| Lung | 0.0 | 0.0 | CNS cancer | 3.0 | 0.0 |
| | | | (glio/astro) U-118- | | |
| | | | MG | | |
| Fetal Lung | 0.0 | 0.0 | CNS cancer | 0.0 | 0.0 |
| | | | (neuro;met) SK-N- | | |
| | | | AS | | |
| Lung ca. NCI- | 0.0 | 0.0 | CNS cancer (astro) | 0.0 | 0.0 |
| N417 | | | SF-539 | | |
| Lung ca. LX-1 | 3.6 | 0.0 | CNS cancer (astro) | 20.3 | 0.0 |
| | | | SNB-75 | | |
| Lung ca. NCI- | 7.4 | 0.0 | CNS cancer (glio) | 39.5 | 0.0 |
| H146 | | | SNB-19 | | |
| Lung ca. SHP-77 | 2.4 | 0.0 | CNS cancer (glio) | 0.0 | 0.0 |
| | | | SF-295 | | |
| Lung ca. A549 | 1.8 | 0.0 | Brain (Amygdala) | 0.0 | 0.0 |
| | | | Pool | | |
| Lung ca. NCI- | 0.0 | 0.0 | Brain (cerebellum) | 0.0 | 0.0 |
| H526 | | | | | |
| Lung ca. NCI-H23 | 0.0 | 0.0 | Brain (fetal) | 0.0 | 0.0 |
| Lung ca. NCI- | 0.0 | 0.0 | Brain (Hippocampus) | 3.2 | 0.0 |
| H460 | | | Pool | | |
| Lung ca. HOP-62 | 0.0 | 100.0 | Cerebral Cortex Pool | 0.0 | 0.0 |
| Lung ca. NCI- | 0.0 | 0.0 | Brain (Substantia | 0.0 | 0.0 |
| H522 | | | nigra) Pool | | |
| Liver | 0.0 | 0.0 | Brain (Thalamus) | 2.3 | 0.0 |
| | | | Pool | | |
| Fetal Liver | 2.2 | 0.0 | Brain (whole) | 12.9 | 0.0 |
| Liver ca. HepG2 | 0.0 | 0.0 | Spinal Cord Pool | 0.0 | 0.0 |
| Kidney Pool | 0.0 | 0.0 | Adrenal Gland | 0.0 | 0.0 |
| Fetal Kidney | 10.4 | 0.0 | Pituitary gland Pool | 0.0 | 0.0 |
| Renal ca. 786-0 | 0.0 | 0.0 | Salivary Gland | 7.2 | 0.0 |
| Renal ca. A498 | 0.0 | 0.0 | Thyroid (female) | 3.5 | 0.0 |
| Renal ca. ACHN | 0.0 | 0.0 | Pancreatic ca. | 0.0 | 0.0 |
| | | | CAPAN2 | | |
| Renal ca. UO-31 | 0.0 | 0.0 | Pancreas Pool | 0.0 | 0.0 |

TABLE BJE
Panel 4.1D

| | Rel. Exp. (%) Ag4117, Run | | Rel. Exp. (%) Ag4117, Run |
|---|---|---|---|
| Tissue Name | 172774997 | Tissue Name | 172774997 |
| Secondary Th1 act | 19.9 | HUVEC IL-1 beta | 10.6 |
| Secondary Th2 act | 32.5 | HUVEC IFN gamma | 100.0 |
| Secondary Tr1 act | 31.0 | HUVEC TNF alpha + IFN gamma | 13.1 |
| Secondary Th1 rest | 17.7 | HUVEC TNF alpha + IL4 | 21.3 |
| Secondary Th2 rest | 22.4 | HUVEC IL-11 | 11.7 |
| Secondary Tr1 rest | 19.6 | Lung Microvascular EC none | 19.6 |
| Primary Th1 act | 26.6 | Lung Microvascular EC TNF alpha + | 35.6 |
| | | IL-1 beta | |
| Primary Th2 act | 33.2 | Microvascular Dermal EC none | 19.6 |
| Primary Tr1 act | 27.4 | Microsvasular Dermal EC | 32.8 |
| | | TNF alpha + IL-1 beta | |
| Primary Th1 rest | 23.5 | Bronchial epithelium TNF alpha + | 22.5 |
| | | IL1 beta | |
| Primary Th2 rest | 22.5 | Small airway epithelium none | 23.0 |
| Primary Tr1 rest | 25.0 | Small airway epithelium TNF alpha + | 14.0 |
| | | IL-1 beta | |
| CD45RA CD4 lymphocyte | 34.2 | Coronery artery SMC rest | 21.6 |
| act | | | |
| CD45RO CD4 lymphocyte | 39.2 | Coronery artery SMC TNF alpha + | 18.6 |
| act | | IL-1 beta | |
| CD8 lymphocyte act | 43.2 | Astrocytes rest | 31.9 |
| Secondary CD8 lymphocyte | 27.0 | Astrocytes TNF alpha + IL-1 beta | 27.4 |
| rest | | | |
| Secondary CD8 lymphocyte | 27.5 | KU-812 (Basophil) rest | 30.6 |
| act | | | |
| CD4 lymphocyte none | 22.1 | KU-812 (Basophil) | 33.2 |
| | | PMA/ionomycin | |
| 2ry Th1/Th2/Tr1_anti-CD95 | 30.8 | CCD1106 (Keratinocytes) none | 13.8 |
| CH11 | | | |
| LAK cells rest | 38.7 | CCD1106 (Keratinocytes) | 16.8 |
| | | TNF alpha + IL-1 beta | |
| LAK cells IL-2 | 33.4 | Liver cirrhosis | 22.4 |
| LAK cells IL-2 + IL-12 | 25.7 | NCI-H292 none | 23.2 |
| LAK cells IL-2 + IFN gamma | 20.4 | NCI-H292 IL-4 | 29.9 |
| LAK cells IL-2 + IL-18 | 27.2 | NCI-H292 IL-9 | 14.9 |
| LAK cells PMA/ionomycin | 19.8 | NCI-H292 IL-13 | 33.0 |
| NK Cells IL-2 rest | 36.3 | NCI-H292 IFN gamma | 14.7 |
| Two Way MLR 3 day | 28.7 | HPAEC none | 20.6 |
| Two Way MLR 5 day | 16.8 | HPAEC TNF alpha + IL-1 beta | 24.0 |
| Two Way MLR 7 day | 9.1 | Lung fibroblast none | 24.0 |
| PBMC rest | 28.5 | Lung fibroblast TNF alpha + IL-1 | 26.8 |
| | | beta | |
| PBMC PWM | 22.4 | Lung fibroblast IL-4 | 20.0 |
| PBMC PHA-L | 17.2 | Lung fibroblast IL-9 | 22.4 |
| Ramos (B cell) none | 32.3 | Lung fibroblast IL-13 | 23.5 |
| Ramos (B cell) ionomycin | 22.2 | Lung fibroblast IFN gamma | 23.7 |
| B lymphocytes PWM | 25.2 | Dermal fibroblast CCD1070 rest | 26.8 |
| B lymphocytes CD40L and | 23.5 | Dermal fibroblast CCD1070 TNF | 21.9 |
| IL-4 | | alpha | |
| EOL-1 dbcAMP | 22.1 | Dermal fibroblast CCD1070 IL-1 | 36.1 |
| | | beta | |
| EOL-1 dbcAMP | 27.7 | Dermal fibroblast IFN gamma | 39.5 |
| PMA/ionomycin | | | |
| Dendritic cells none | 26.1 | Dermal fibroblast IL-4 | 23.2 |
| Dendritic cells LPS | 26.8 | Dermal Fibroblasts rest | 11.3 |
| Dendritic cells anti-CD40 | 30.8 | Neutrophils TNFa + LPS | 21.5 |
| Monocytes rest | 31.2 | Neutrophils rest | 39.0 |
| Monocytes LPS | 27.0 | Colon | 45.4 |
| Macrophages rest | 27.0 | Lung | 34.9 |
| Macrophages LPS | 21.5 | Thymus | 8.0 |
| HUVEC none | 25.2 | Kidney | 24.7 |
| HUVEC starved | 14.2 | | |

CNS_neurodegeneration_v1.0 Summary: Ag4117 This panel demonstrates the expression of the CG57823-01 gene at low levels in the brains of several individuals. However, no differential expression of this gene was detected between Alzheimer's diseased postmortem brains and those of non-demented controls in this experiment. The CG57823-01 gene encodes a protein with homology to acyltransferase. Ag3514 Expression of this gene is low/undetectable (CTs>35) across all of the samples on this panel (data not shown).

General_screening_panel_v1.4 Summary: Ag3514 In one experiment with this probe/primer set, expression of the CG57823-01 gene is limited to a bone marrow sample (CT=33.9). Thus, expression of this gene may be used to distinguish bone marrow from the other samples on this panel. In the other experiment using this probe/primer set, expression of this gene is low/undetectable (CTs>35) across all of the samples on this panel (data not shown).

Panel 4.1D Summary: Ag4117 Expression of the CG57823-01 gene is highest in IFN gamma treated human umbilical vein endothelial cells (CT=33). This gene is expressed at low levels in a wide range of cell types of significance in the immune response in health and disease, including T-cells, B-cells, endothelial cells, macrophages, monocytes, dendritic cells, basophils, eosinophils and peripheral blood mononuclear cells, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. Therefore, therapeutic modulation of the activity of this gene or its protein product may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 4D Summary: Ag3514 Expression of this gene is low/undetectable (CTs>35) across all of the samples on this panel (data not shown).

NOV68

Expression of NOV68/CG57801-01 was assessed using the primer-probe sets Ag3335 and Ag3336, described in Tables BKA and BKB. Results of the RTQ-PCR runs are shown in Tables BKC, BKD, BKE and BKF.

TABLE BKA
Probe Name Ag3335

| Primers | Sequences | Length | Start Position | Seq id no: |
|---|---|---|---|---|
| Forward | 5′-ttgccatctattccgagtactg-3′ | 22 | 477 | 458 |
| Probe | TET-5′-ccaacctcatgaagcagggcaagt-3′-TAMRA | 24 | 459 | 459 |
| Reverse | 5′-caggcttcaaagaaatgtctgt-3′ | 22 | 555 | 460 |

TABLE BKB
Probe Name Ag3336

| Primers | Sequences | Length | Start Position | Seq id no: |
|---|---|---|---|---|
| Forward | 5′-gtgcaagaggacaaggagatg-3′ | 21 | 1181 | 461 |
| Probe | TET-5′-ttcagaaaaccagaagaaacttgcca-3′-TAMRA | 26 | 1213 | 462 |
| Reverse | 5′-cctgccttttgagcatttaac-3′ | 21 | 1240 | 463 |

TABLE BKC
CNS_neurodegeneration_v1.0

| | Rel. Exp. (%) | Rel. Exp. (%) | Rel. Exp. (%) | | Rel. Exp. (%) | Rel. Exp. (%) | Rel. Exp. (%) |
|---|---|---|---|---|---|---|---|
| Tissue | Ag3335, Run | Ag3335, Run | Ag3336, Run | Tissue | Ag3335, Run | Ag3335, Run | Ag3336, Run |
| Name | 210138107 | 230512529 | 210138110 | Name | 210138107 | 230512529 | 210138110 |
| AD 1 Hippo | 26.1 | 22.8 | 28.1 | Control | 5.3 | 4.3 | 6.1 |
| | | | | (Path) 3 | | | |
| | | | | Temporal | | | |
| | | | | Ctx | | | |
| AD 2 Hippo | 29.9 | 31.9 | 29.5 | Control | 18.3 | 15.8 | 17.3 |
| | | | | (Path) 4 | | | |
| | | | | Temporal | | | |
| | | | | Ctx | | | |
| AD 3 Hippo | 13.7 | 11.8 | 11.7 | AD 1 | 11.7 | 12.1 | 15.6 |
| | | | | Occipital | | | |
| | | | | Ctx | | | |
| AD 4 Hippo | 13.4 | 8.3 | 10.4 | AD 2 | 0.0 | 0.0 | 0.0 |
| | | | | Occipital | | | |
| | | | | Ctx | | | |
| | | | | (Missing) | | | |
| AD 5 Hippo | 39.8 | 39.0 | 30.8 | AD 3 | 11.6 | 8.5 | 7.9 |
| | | | | Occipital | | | |
| | | | | Ctx | | | |
| AD 6 Hippo | 100.0 | 99.3 | 100.0 | AD 4 | 25.5 | 15.1 | 12.1 |
| | | | | Occipital | | | |
| | | | | Ctx | | | |
| Control 2 | 40.3 | 27.4 | 44.8 | AD 5 | 25.5 | 26.8 | 27.4 |
| Hippo | | | | Occipital | | | |
| | | | | Ctx | | | |
| Control 4 | 26.1 | 25.0 | 27.7 | AD 6 | 27.4 | 22.2 | 27.9 |
| Hippo | | | | Occipital | | | |
| | | | | Ctx | | | |
| Control | 15.9 | 7.3 | 12.8 | Control 1 | 4.9 | 4.6 | 5.8 |
| (Path) 3 | | | | Occipital | | | |
| Hippo | | | | Ctx | | | |
| AD 1 | 28.1 | 24.7 | 33.2 | Control 2 | 31.6 | 26.8 | 43.2 |
| Temporal | | | | Occipital | | | |
| Ctx | | | | Ctx | | | |
| AD 2 | 26.8 | 21.9 | 23.5 | Control 3 | 13.7 | 13.8 | 6.0 |
| Temporal | | | | Occipital | | | |
| Ctx | | | | Ctx | | | |
| AD 3 | 6.2 | 8.3 | 6.7 | Control 4 | 11.7 | 11.7 | 11.6 |
| Temporal | | | | Occipital | | | |
| Ctx | | | | Ctx | | | |
| AD 4 | 27.5 | 22.4 | 21.9 | Control | 50.7 | 48.0 | 48.0 |
| Temporal | | | | (Path) 1 | | | |
| Ctx | | | | Occipital | | | |
| | | | | Ctx | | | |
| AD 5 Inf | 84.1 | 80.7 | 63.7 | Control | 13.2 | 7.9 | 7.8 |
| Temporal | | | | (Path) 2 | | | |
| Ctx | | | | Occipital | | | |
| | | | | Ctx | | | |
| AD 5 Sup | 52.9 | 58.6 | 52.9 | Control | 4.0 | 4.2 | 3.9 |
| Temporal | | | | (Path) 3 | | | |
| Ctx | | | | Occipital | | | |
| | | | | Ctx | | | |
| AD 6 Inf | 95.9 | 100.0 | 97.3 | Control | 11.0 | 11.3 | 10.8 |
| Temporal | | | | (Path) 4 | | | |
| Ctx | | | | Occipital | | | |
| | | | | Ctx | | | |
| AD 6 Sup | 67.4 | 67.4 | 54.3 | Control 1 | 11.8 | 7.3 | 9.9 |
| Temporal | | | | Parietal Ctx | | | |
| Ctx | | | | | | | |
| Control 1 | 8.4 | 6.8 | 8.0 | Control 2 | 48.6 | 37.6 | 49.7 |
| Temporal | | | | Parietal Ctx | | | |
| Ctx | | | | | | | |
| Control 2 | 25.2 | 31.0 | 25.0 | Control 3 | 22.7 | 12.8 | 13.6 |
| Temporal | | | | Parietal Ctx | | | |
| Ctx | | | | | | | |
| Control 3 | 15.9 | 16.3 | 13.5 | Control | 27.0 | 24.7 | 28.1 |
| Temporal | | | | (Path) 1 | | | |
| Ctx | | | | Parietal Ctx | | | |
| Control 3 | 12.1 | 8.1 | 9.4 | Control | 19.8 | 16.7 | 16.3 |
| Temporal | | | | (Path) 2 | | | |
| Ctx | | | | Parietal Ctx | | | |
| Control | 26.2 | 27.5 | 30.6 | Control | 4.0 | 3.8 | 0.4 |
| (Path) 1 | | | | (Path) 3 | | | |
| Temporal | | | | Parietal Ctx | | | |
| Ctx | | | | | | | |
| Control | 19.5 | 20.6 | 13.3 | Control | 27.4 | 23.7 | 25.2 |
| (Path) 2 | | | | (Path) 4 | | | |
| Temporal | | | | Parietal Ctx | | | |
| Ctx | | | | | | | |

TABLE BKD
General_screening_panel_v1.4

| | Rel. Exp. (%) | Rel. Exp. (%) | | Rel. Exp. (%) | Rel. Exp. (%) |
|---|---|---|---|---|---|
| | Ag3335, Run | Ag3336, Run | | Ag3335, Run | Ag3336, Run |
| Tissue Name | 213333209 | 215773745 | Tissue Name | 213333209 | 215773745 |
| Adipose | 4.0 | 4.6 | Renal ca. TK-10 | 12.2 | 12.2 |
| Melanoma* | 2.5 | 2.0 | Bladder | 24.0 | 19.8 |
| Hs688(A).T | | | | | |
| Melanoma* | 0.8 | 0.7 | Gastric ca. (liver | 29.5 | 21.6 |
| Hs688(B).T | | | met.) NCI-N87 | | |
| Melanoma* M14 | 30.1 | 28.9 | Gastric ca. KATO III | 52.5 | 41.2 |
| Melanoma* | 2.4 | 2.4 | Colon ca. SW-948 | 4.3 | 6.2 |
| LOXIMVI | | | | | |
| Melanoma* SK- | 7.5 | 12.0 | Colon ca. SW480 | 100.0 | 100.0 |
| MEL-5 | | | | | |
| Squamous cell | 1.9 | 1.8 | Colon ca.* (SW480 | 49.3 | 57.4 |
| carcinoma SCC-4 | | | met) SW620 | | |
| Testis Pool | 5.1 | 4.1 | Colon ca. HT29 | 12.7 | 13.4 |
| Prostate ca.* | 9.6 | 7.3 | Colon ca. HCT-116 | 14.2 | 16.7 |
| (bone met) PC-3 | | | | | |
| Prostate Pool | 11.4 | 9.3 | Colon ca. CaCo-2 | 13.2 | 9.9 |
| Placenta | 13.9 | 9.5 | Colon cancer tissue | 20.3 | 23.5 |
| Uterus Pool | 2.9 | 2.1 | Colon ca. SW1116 | 5.6 | 7.5 |
| Ovarian ca. | 6.4 | 4.9 | Colon ca. Colo-205 | 32.1 | 39.2 |
| OVCAR-3 | | | | | |
| Ovarian ca. SK- | 12.5 | 12.5 | Colon ca. SW-48 | 10.9 | 10.6 |
| OV-3 | | | | | |
| Ovarian ca. | 2.6 | 2.0 | Colon Pool | 6.9 | 5.2 |
| OVCAR-4 | | | | | |
| Ovarian ca. | 15.1 | 15.3 | Small Intestine Pool | 6.1 | 6.5 |
| OVCAR-5 | | | | | |
| Ovarian ca. | 7.5 | 6.6 | Stomach Pool | 7.6 | 4.1 |
| IGROV-1 | | | | | |
| Ovarian ca. | 2.5 | 2.6 | Bone Marrow Pool | 2.0 | 2.5 |
| OVCAR-8 | | | | | |
| Ovary | 9.5 | 8.4 | Fetal Heart | 3.3 | 1.7 |
| Breast ca. MCF-7 | 27.9 | 20.6 | Heart Pool | 3.0 | 4.8 |
| Breast ca. MDA- | 5.7 | 6.2 | Lymph Node Pool | 4.9 | 5.7 |
| MB-231 | | | | | |
| Breast ca. BT 549 | 3.8 | 3.5 | Fetal Skeletal Muscle | 0.8 | 0.7 |
| Breast ca. T47D | 23.2 | 32.1 | Skeletal Muscle Pool | 1.9 | 1.4 |
| Breast ca. MDA-N | 9.6 | 9.8 | Spleen Pool | 49.0 | 39.0 |
| Breast Pool | 6.9 | 5.6 | Thymus pool | 9.6 | 0.0 |
| Trachea | 10.6 | 6.5 | CNS cancer | 2.1 | 3.2 |
| | | | (glio/astro) U87-MG | | |
| Lung | 2.8 | 2.9 | CNS cancer | 6.7 | 4.4 |
| | | | (glio/astro) U-118- | | |
| | | | MG | | |
| Fetal Lung | 11.3 | 10.5 | CNS cancer | 0.6 | 0.2 |
| | | | (neuro;met) SK-N- | | |
| | | | AS | | |
| Lung ca. NCI- | 2.6 | 2.9 | CNS cancer (astro) | 1.4 | 1.5 |
| N417 | | | SF-539 | | |
| Lung ca. LX-1 | 79.0 | 92.0 | CNS cancer (astro) | 6.6 | 11.3 |
| | | | SNB-75 | | |
| Lung ca. NCI- | 2.0 | 2.7 | CNS cancer (glio) | 5.8 | 7.3 |
| H146 | | | SNB-19 | | |
| Lung ca. SHP-77 | 0.0 | 0.0 | CNS cancer (glio) | 29.7 | 18.0 |
| | | | SF-295 | | |
| Lung ca. A549 | 5.0 | 5.4 | Brain (Amygdala) | 14.9 | 9.0 |
| | | | Pool | | |
| Lung ca. NCI- | 2.4 | 3.2 | Brain (cerebellum) | 7.7 | 6.9 |
| H526 | | | | | |
| Lung ca. NCI-H23 | 2.3 | 2.4 | Brain (fetal) | 10.2 | 7.6 |
| Lung ca. NCI- | 1.8 | 2.4 | Brain (Hippocampus) | 13.6 | 9.5 |
| H460 | | | Pool | | |
| Lung ca. HOP-62 | 1.7 | 1.2 | Cerebral Cortex Pool | 12.6 | 7.6 |
| Lung ca. NCI- | 1.1 | 0.8 | Brain (Substantia | 14.9 | 9.5 |
| H522 | | | nigra) Pool | | |
| Liver | 1.6 | 1.0 | Brain (Thalamus) | 19.5 | 12.3 |
| | | | Pool | | |
| Fetal Liver | 6.3 | 15.2 | Brain (whole) | 9.0 | 7.2 |
| Liver ca. HepG2 | 7.6 | 7.7 | Spinal Cord Pool | 21.0 | 16.2 |
| Kidney Pool | 10.6 | 8.5 | Adrenal Gland | 12.3 | 7.2 |
| Fetal Kidney | 11.0 | 10.6 | Pituitary gland Pool | 2.9 | 1.8 |
| Renal ca. 786-0 | 7.1 | 7.7 | Salivary Gland | 3.1 | 3.0 |
| Renal ca. A498 | 1.8 | 2.9 | Thyroid (female) | 5.0 | 4.9 |
| Renal ca. ACHN | 4.0 | 3.4 | Pancreatic ca. | 14.0 | 12.6 |
| | | | CAPAN2 | | |
| Renal ca. UO-31 | 1.9 | 2.0 | Pancreas Pool | 13.4 | 11.9 |

TABLE BKE
Panel 2.2

| | Rel. Exp. (%) Ag3335, Run | | Rel. Exp. (%) Ag3335, Run |
|---|---|---|---|
| Tissue Name | 173762744 | Tissue Name | 173762744 |
| Normal Colon | 12.8 | Kidney Margin (OD04348) | 100.0 |
| Colon cancer (OD06064) | 12.2 | Kidney malignant cancer | 5.7 |
| | | (OD06204B) | |
| Colon Margin (OD06064) | 8.1 | Kidney normal adjacent tissue | 9.7 |
| | | (OD06204E) | |
| Colon cancer (OD06159) | 1.6 | Kidney Cancer (OD04450-01) | 35.4 |
| Colon Margin (OD06159) | 10.6 | Kidney Margin (OD04450-03) | 25.5 |
| Colon cancer (OD06297-04) | 3.8 | Kidney Cancer 8120613 | 1.8 |
| Colon Margin (OD06297-05) | 11.4 | Kidney Margin 8120614 | 10.7 |
| CC Gr.2 ascend colon | 8.0 | Kidney Cancer 9010320 | 1.8 |
| (ODO3921) | | | |
| CC Margin (ODO3921) | 3.1 | Kidney Margin 9010321 | 6.7 |
| Colon cancer metastasis | 6.6 | Kidney Cancer 8120607 | 6.1 |
| (OD06104) | | | |
| Lung Margin (OD06104) | 4.5 | Kidney Margin 8120608 | 3.5 |
| Colon mets to lung (OD04451- | 4.8 | Normal Uterus | 4.3 |
| 01) | | | |
| Lung Margin (OD04451-02) | 21.3 | Uterine Cancer 064011 | 6.6 |
| Normal Prostate | 5.8 | Normal Thyroid | 1.7 |
| Prostate Cancer (OD04410) | 11.5 | Thyroid Cancer 064010 | 3.4 |
| Prostate Margin (OD04410) | 9.5 | Thyroid Cancer A302152 | 9.7 |
| Normal Ovary | 11.0 | Thyroid Margin A302153 | 6.6 |
| Ovarian cancer (OD06283-03) | 7.3 | Normal Breast | 15.2 |
| Ovarian Margin (OD06283-07) | 6.5 | Breast Cancer (OD04566) | 3.2 |
| Ovarian Cancer 064008 | 6.6 | Breast Cancer 1024 | 12.1 |
| Ovarian cancer (OD06145) | 10.3 | Breast Cancer (OD04590-01) | 5.8 |
| Ovarian Margin (OD06145) | 12.4 | Breast Cancer Mets (OD04590- | 9.6 |
| | | 03) | |
| Ovarian cancer (OD06455-03) | 5.8 | Breast Cancer Metastasis | 22.8 |
| | | (OD04655-05) | |
| Ovarian Margin (OD06455-07) | 1.7 | Breast Cancer 064006 | 14.1 |
| Normal Lung | 13.1 | Breast Cancer 9100266 | 16.7 |
| Invasive poor diff. lung adeno | 6.7 | Breast Margin 9100265 | 7.6 |
| (ODO4945-01 | | | |
| Lung Margin (ODO4945-03) | 24.1 | Breast Cancer A209073 | 4.6 |
| Lung Malignant Cancer | 12.8 | Breast Margin A2090734 | 15.8 |
| (OD03126) | | | |
| Lung Margin (OD03126) | 5.4 | Breast cancer (OD06083) | 12.4 |
| Lung Cancer (OD05014A) | 11.6 | Breast cancer node metastasis | 12.8 |
| | | (OD06083) | |
| Lung Margin (OD05014B) | 16.4 | Normal Liver | 16.7 |
| Lung cancer (OD06081) | 2.0 | Liver Cancer 1026 | 2.0 |
| Lung Margin (OD06081) | 10.2 | Liver Cancer 1025 | 13.6 |
| Lung Cancer (OD04237-01) | 5.9 | Liver Cancer 6004-T | 11.8 |
| Lung Margin (OD04237-02) | 21.8 | Liver Tissue 6004-N | 6.7 |
| Ocular Melanoma Metastasis | 1.2 | Liver Cancer 6005-T | 9.7 |
| Ocular Melanoma Margin | 7.4 | Liver Tissue 6005-N | 13.5 |
| (Liver) | | | |
| Melanoma Metastasis | 16.8 | Liver Cancer 064003 | 16.6 |
| Melanoma Margin (Lung) | 24.7 | Normal Bladder | 14.1 |
| Normal Kidney | 9.3 | Bladder Cancer 1023 | 9.1 |
| Kidney Ca, Nuclear grade 2 | 45.1 | Bladder Cancer A302173 | 4.5 |
| (OD04338) | | | |
| Kidney Margin (OD04338) | 9.5 | Normal Stomach | 33.0 |
| Kidney Ca Nuclear grade 1/2 | 72.2 | Gastric Cancer 9060397 | 2.6 |
| (OD04339) | | | |
| Kidney Margin (OD04339) | 18.7 | Stomach Margin 9060396 | 11.3 |
| Kidney Ca, Clear cell type | 7.2 | Gastric Cancer 9060395 | 8.7 |
| (OD04340) | | | |
| Kidney Margin (OD04340) | 18.3 | Stomach Margin 9060394 | 11.4 |
| Kidney Ca, Nuclear grade 3 | 2.8 | Gastric Cancer 064005 | 18.3 |
| (OD04348) | | | |

TABLE BKF
Panel 4D

| | Rel. Exp. (%) | Rel. Exp. (%) | | Rel. Exp. (%) | Rel. Exp. (%) |
|---|---|---|---|---|---|
| | Ag3335, Run | Ag3336, Run | | Ag3335, Run | Ag3336, Run |
| Tissue Name | 165128064 | 165128065 | Tissue Name | 165128064 | 165128065 |
| Secondary Th1 act | 32.1 | 14.0 | HUVEC IL-1beta | 2.7 | 0.4 |
| Secondary Th2 act | 42.6 | 14.6 | HUVEC IFN gamma | 3.3 | 0.8 |
| Secondary Tr1 act | 43.5 | 11.6 | HUVEC TNF alpha + | 5.8 | 0.8 |
| | | | IFN gamma | | |
| Secondary Th1 rest | 9.0 | 3.1 | HUVEC TNF alpha + | 7.5 | 1.7 |
| | | | IL4 | | |
| Secondary Th2 rest | 15.5 | 4.0 | HUVEC IL-11 | 1.6 | 0.5 |
| Secondary Tr1 rest | 15.4 | 3.3 | Lung Microvascular EC | 2.7 | 0.4 |
| | | | none | | |
| Primary Th1 act | 38.2 | 9.3 | Lung Microvascular EC | 1.7 | 0.4 |
| | | | TNF alpha + IL-1beta | | |
| Primary Th2 act | 34.4 | 8.1 | Microvascular Dermal | 2.8 | 0.7 |
| | | | EC none | | |
| Primary Tr1 act | 40.6 | 0.2 | Microvascular Dermal | 2.5 | 0.6 |
| | | | EC TNF alpha + IL- | | |
| | | | 1beta | | |
| Primary Th1 rest | 62.4 | 15.5 | Bronchial epithelium | 0.5 | 0.2 |
| | | | TNF alpha + IL-1beta | | |
| Primary Th2 rest | 41.8 | 100.0 | Small airway epithelium | 0.8 | 0.2 |
| | | | none | | |
| Primary Tr1 rest | 29.3 | 6.3 | Small airway epithelium | 2.6 | 0.3 |
| | | | TNF alpha + IL-1beta | | |
| CD45RA CD4 | 20.4 | 5.3 | Coronery artery SMC | 5.4 | 1.2 |
| lymphocyte act | | | rest | | |
| CD45RO CD4 | 45.7 | 11.0 | Coronery artery SMC | 3.4 | 0.8 |
| lymphocyte act | | | TNF alpha + IL-1beta | | |
| CD8 lymphocyte act | 41.8 | 7.7 | Astrocytes rest | 3.5 | 0.8 |
| Secondary CD8 | 42.3 | 6.4 | Astrocytes TNF alpha + | 6.4 | 1.5 |
| lymphocyte rest | | | IL-1beta | | |
| Secondary CD8 | 28.5 | 5.0 | KU-812 (Basophil) rest | 6.3 | 1.0 |
| lymphocyte act | | | | | |
| CD4 lymphocyte none | 23.5 | 3.4 | KU-812 (Basophil) | 19.9 | 4.5 |
| | | | PMA/ionomycin | | |
| 2ry Th1/Th2/Tr1_anti- | 29.7 | 6.2 | CCD1106 | 1.2 | 0.2 |
| CD95 CH11 | | | (Keratinocytes) none | | |
| LAK cells rest | 39.0 | 6.9 | CCD1106 | 0.1 | 0.1 |
| | | | (Keratinocytes) | | |
| | | | TNF alpha + IL-1beta | | |
| LAK cells IL-2 | 58.2 | 12.8 | Liver cirrhosis | 5.7 | 1.2 |
| LAK cells IL-2 + IL-12 | 47.0 | 7.2 | Lupus kidney | 3.0 | 0.9 |
| LAK cells IL-2 + IFN | 66.4 | 10.5 | NCI-H292 none | 3.8 | 0.8 |
| gamma | | | | | |
| LAK cells IL-2 + IL- | 49.7 | 7.8 | NCI-H292 IL-4 | 5.0 | 0.6 |
| 18 | | | | | |
| LAK cells | 20.3 | 4.5 | NCI-H292 IL-9 | 5.1 | 2.6 |
| PMA/ionomycin | | | | | |
| NK Cells IL-2 rest | 47.3 | 9.7 | NCI-H292 IL-13 | 3.2 | 0.4 |
| Two Way MLR 3 day | 49.3 | 9.7 | NCI-H292 IFN gamma | 1.6 | 0.5 |
| Two Way MLR 5 day | 28.1 | 6.7 | HPAEC none | 1.7 | 0.2 |
| Two Way MLR 7 day | 26.6 | 5.8 | HPAEC TNF alpha + | 3.4 | 0.8 |
| | | | IL-1beta | | |
| PBMC rest | 22.1 | 3.3 | Lung fibroblast none | 6.7 | 2.1 |
| PBMC PWM | 100.0 | 25.0 | Lung fibroblast TNF | 6.4 | 2.0 |
| | | | alpha + IL-1beta | | |
| PBMC PHA-L | 41.8 | 8.0 | Lung fibroblast IL-4 | 45.7 | 13.4 |
| Ramos (B cell) none | 4.3 | 0.7 | Lung fibroblast IL-9 | 17.2 | 2.1 |
| Ramos (B cell) | 17.9 | 2.4 | Lung fibroblast IL-13 | 32.8 | 8.8 |
| ionomycin | | | | | |
| B lymphocytes PWM | 79.6 | 11.0 | Lung fibroblast IFN | 51.8 | 9.9 |
| | | | gamma | | |
| B lymphocytes | 22.2 | 3.9 | Dermal fibroblast | 7.3 | 1.8 |
| CD40L and IL-4 | | | CCD1070 rest | | |
| EOL-1 dbcAMP | 1.8 | 0.4 | Dermal fibroblast | 42.9 | 13.4 |
| | | | CCD1070 TNF alpha | | |
| EOL-1 dbcAMP | 13.0 | 2.4 | Dermal fibroblast | 8.4 | 2.3 |
| PMA/ionomycin | | | CCD1070 IL-1beta | | |
| Dendritic cells none | 13.2 | 1.8 | Dermal fibroblast IFN | 9.0 | 2.8 |
| | | | gamma | | |
| Dendritic cells LPS | 21.0 | 4.0 | Dermal fibroblast IL-4 | 12.4 | 3.5 |
| Dendritic cells anti- | 8.7 | 1.9 | IBD Colitis 2 | 1.7 | 0.4 |
| CD40 | | | | | |
| Monocytes rest | 16.2 | 2.4 | IBD Crohn's | 3.2 | 0.8 |
| Monocytes LPS | 12.8 | 3.8 | Colon | 17.7 | 4.5 |
| Macrophages rest | 37.6 | 7.2 | Lung | 10.6 | 2.8 |
| Macrophages LPS | 21.5 | 3.0 | Thymus | 35.1 | 13.7 |
| HUVEC none | 4.5 | 0.9 | Kidney | 27.2 | 8.6 |
| HUVEC starved | 12.0 | 1.6 | | | |

CNS_neurodegeneration_v1.0 Summary: Ag3335/Ag3336 Results from three experiments using two different probe/primer sets are in excellent agreement and when analyzed by ANCOVA p=0.01 and p=0.01. This panel confirms the expression of the CG57801-01 gene at moderate levels in the brains of an independent group of individuals. This gene is found to be upregulated in the temporal cortex of Alzheimer's disease patients. The CG57801-01 gene encodes a protein with homology to guanine nucleotide exchange factor. Inhibition of the CG57801-01 gene or its protein product may decrease neuronal death and be of use in the treatment of Alzheimer's disease.

General_screening_panel_v1.4 Summary: Ag3335/Ag3336 Results from two experiments using two different probe/primer sets are in excellent agreement. Expression of the CG57801-01 gene appears to be upregulated in a number of colon cancer cell lines, when compared to normal colon. Therefore, expression of this gene may be used to distinguish colon cancer cell lines from the other samples on this panel. Furthermore, therapeutic modulation of the activity of this gene or its protein product, using small molecule drugs, antibodies, and protein therapeutics, may be of use in the treatment of colon cancer. These results are consistent with what is observed in Panel 2.2. The CG57801-01 gene encodes a putative guanine nucleotide exchange factor (GEF) that is homologous to the ASEF protein. ASEF is known to stimulate cell flattening, membrane ruffling, and lamellipodia formation in the presence of APC (adenomatous polyposis coli protein), suggesting that the APC-Asef complex may regulate the actin cytoskeletal network, cell morphology and migration, and neuronal function (ref. 1). The adenomatous polyposis coli gene (APC) is mutated in familial adenomatous polyposis and in sporadic colorectal tumors.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression. The CG57801-01 gene also displays significant homology to the collybistin gene. Collybistin is a newly identified brain-specific GEF that induces submembrane clustering of gephyrin and may be an important determinant of inhibitory postsynaptic membrane formation and plasticity (ref. 2).

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes. This gene is well-expressed in pancreas, which includes the insulin-secreting beta cells in the islets of Langerhans. Guanine nucleotide exchange factors are present in beta cells and are activated by CAMP. A rise in cAMP levels precedes glucose-induced insulin secretion (Ref. 3). Thus, the CG57801-01 gene may be a target for therapeutic modulation of the beta cell secretory defect in Type 2 diabetes.

References:

1. Kawasaki Y, Senda T, Ishidate T, Koyama R, Morishita T, twayama Y, Fliguchi O, Akiyama T. (2000) Asef, a link between the tumor suppressor APC and G-protein signaling. Science 289(5482): 1194-7

The adenomatous polyposis coli gene (APC) is mutated in familial adenomatous polyposis and in sporadic colorectal tumors. Here the APC gene product is shown to bind through its armadillo repeat domain to a Rac-specific guanine nucleotide exchange factor (GEF), termed Asef. Endogenous APC colocalized with Asef in mouse colon epithelial cells and neuronal cells. Furthermore, APC enhanced the GEF activity of Asef and stimulated Asef-mediated cell flattening, membrane ruffling, and lamellipodia formation in MDCK cells. These results suggest that the APC-Asef complex may regulate the actin cytoskeletal network, cell morphology and migration, and neuronal function.

PMID: 10947987

2. Kins S, Betz H, Kirsch J (2000) Collybistin, a newly identified brain-specific GEF, induces submembrane clustering of gephyrin. Nat Neurosci 3(1):22-9

The formation of postsynaptic GABAA and glycine receptor clusters requires the receptor-associated peripheral membrane protein gephyrin. Here we describe two splice variants of a novel gephyrin-binding protein, termed collybistin I and II, which belong to the family of dbl-like GDP/GTP exchange factors (GEFs). Co-expression of collybistin II with gephyrin induced the formation of submembrane gephyrin aggregates that accumulate hetero-oligomeric glycine receptors. Our data suggest that collybistin II regulates the membrane deposition of gephyrin by activating a GTPase of the Rho/Rac family. Therefore, this protein may be an important determinant of inhibitory postsynaptic membrane formation and plasticity.

PMID: 10607391

3. Leech C A, Holz G G, Chepurny O, Habener J F. (2000) Expression of cAMP-regulated guanine nucleotide exchange factors in pancreatic beta-cells. Biochem Biophys Res Commun. 278(1):44-7.

The insulinotropic hormone glucagon-like peptide-1 (GLP-1) binds to a Gs-coupled receptor on pancreatic beta-cells and potentiates glucose-induced insulin secretion, insulin gene transcription, and beta-cell growth. These stimulatory effects have been attributed to the elevation of intracellular cAMP levels, though it is now apparent that some stimulatory effects of GLP-1 occur independently of the cAMP-mediated activation of protein kinase A (PKA). The nature of this alternative, PKA-independent signaling pathway remains unknown. Mere we present evidence for the expression of type 1 and type 2 cAMP-regulated guanine nucleotide exchange factors (cAMP-GEFs) in beta-cells. GEFs are activated by their binding of cAMP. Because cAMP-GEFs activate Ras/MAPK proliferation signaling pathways, they may play an important role in PKA-independent, GLP-1-mediated, signaling pathways in the regulation of beta-cell growth and differentiation.

PMID: 11071853

Panel 2.2 Summary: Ag3335 The CG57801-01 gene encodes a protein with homology to APC-stimulated guanine nucleotide exchange factor (ASEF). Its expression is low to moderate in all of the samples on this panel. Interestingly, expression of this gene is lower in some colon, lung and kidney cancer tissues compared to matched normal tissues. This observation suggests that expression of this gene can be used to distinguish these cancers from normal tissues. Also, therapeutic modulation of the activity of the protein encoded by this gene may be beneficial in the treatment of colon, kidney and lung cancer.

ASEF is known to mediate cell flattening, membrane ruffling, and lamellipodia formation which is stimulated by presence of APC (adenomatous polyposis coli gene) (Ref 1). The adenomatous polyposis coli gene (APC) is mutated in familial adenomatous polyposis and in sporadic colorectal tumors.

References:

1. Kawasaki Y, Senda T, Ishidate T, Koyama R, Morishita T, Iwayama Y, Higuchi O, Akiyama T. (2000) Asef, a link between the tumor suppressor APC and G-protein signaling. Science 289(5482):1194-7

The adenomatous polyposis coli gene (APC) is mutated in familial adenomatous polyposis and in sporadic colorectal tumors. Here the APC gene product is shown to bind through its armadillo repeat domain to a Rac-specific guanine nucleotide exchange factor (GEF), termed Asef. Endogenous APC colocalized with Asef in mouse colon epithelial cells and neuronal cells. Furthermore, APC enhanced the GEF activity of Asef and stimulated Asef-mediated cell flattening, membrane ruffling, and lamellipodia formation in MDCK cells. These results suggest that the APC-Asef complex may regulate the actin cytoskeletal network, cell morphology and migration, and neuronal function.

Panel 4D Summary: Ag3336/Ag3335 The CG57801-01 transcript is expressed in several tissues. It is expressed in most lymphocytes, in both unstimulated and stimulated T cells. The expression of this gene is upregulate in B cells and PBMC treated with pokeweed mitogen. Myeloid cells also express the transcript. Most non-hematopoietic cell types do not consistently express this transcript with the exception of fibroblasts which upregulate the transcript in response to IL-4, IL-] 3, gamma interferon and tnfalpha. The expression profile of this gene suggests that it may be important in the normal function or activation of leukocytes and fibroblasts. Therefore, modulation of the gene product with a functional therapeutic may be useful for immunomodulation, or to treat diseases such as asthma, emphysema, psoriasis, and arthritis.

NOV69

Expression of NOV69/CG57719-01 was assessed using the primer-probe sets Ag3314 and Ag4358, described in Tables BLA and BLB. Results of the RTQ-PCR runs are shown in Tables BLC, BLD and BLE.

TABLE BLA
Probe Name Ag3314

| Primers | Sequences | Length | Start Position | SEQ ID NO |
|---|---|---|---|---|
| Forward | 5′-ggaatacctggtcaggaagaag-3′ | 22 | 1280 | 464 |
| Probe | TET-5′-cacatctatatccccaagaacggtca-3′-TAMRA | 26 | 1302 | 465 |
| Reverse | 5′-ttggcattgatacagctgaagt-3′ | 22 | 1333 | 466 |

TABLE BLB
Probc Name Ag4358

| Primers | Sequences | Length | Start Position | SEQ ID NO |
|---|---|---|---|---|
| Forward | 5′-tgtctcaaggctttgagttctt-3′ | 22 | 901 | 467 |
| Probe | TET-5′-ctgcagccagtctctgtccaaaa-3′-TAMRA | 23 | 923 | 468 |
| Reverse | 5′-tccccactccttcatcataaat-3′ | 22 | 954 | 469 |

TABLE BLC
CNS_neurodegeneration_v1.0

| | Rel. Exp.(%) | Rel. Exp.(%) | | Rel. Exp.(%) | Rel. Exp.(%) |
|---|---|---|---|---|---|
| | Ag3314, Run | Ag4358, Run | | Ag3314, Run | Ag4358, Run |
| Tissue Name | 210138296 | 224372540 | Tissue Name | 210138296 | 224372540 |
| AD 1 Hippo | 4.6 | 25.2 | Control (Path) 3 | 0.0 | 9.7 |
| | | | Temporal Ctx | | |
| AD 2 Hippo | 18.6 | 52.5 | Control (Path) 4 | 74.2 | 39.0 |
| | | | Temporal Ctx | | |
| AD 3 Hippo | 4.1 | 8.5 | AD 1 Occipital | 16.4 | 14.6 |
| | | | Ctx | | |
| AD 4 Hippo | 6.4 | 11.8 | AD 2 Occipital | 0.0 | 0.0 |
| | | | Ctx (Missing) | | |
| AD 5 Hippo | 35.6 | 51.4 | AD 3 Occipital | 7.4 | 8.2 |
| | | | Ctx | | |
| AD 6 Hippo | 61.6 | 95.9 | AD 4 Occipital | 11.7 | 29.7 |
| | | | Ctx | | |
| Control 2 Hippo | 5.8 | 29.3 | AD 5 Occipital | 10.7 | 46.3 |
| | | | Ctx | | |
| Control 4 Hippo | 5.7 | 36.1 | AD 6 Occipital | 5.4 | 37.4 |
| | | | Ctx | | |
| Control (Path) 3 | 0.0 | 9.6 | Control 1 | 0.0 | 8.4 |
| Hippo | | | Occipital Ctx | | |
| AD 1 Temporal | 4.2 | 25.2 | Control 2 | 4.3 | 77.9 |
| Ctx | | | Occipital Ctx | | |
| AD 2 Temporal | 14.9 | 43.2 | Control 3 | 36.6 | 15.3 |
| Ctx | | | Occipital Ctx | | |
| AD 3 Temporal | 0.0 | 5.4 | Control 4 | 0.0 | 14.8 |
| Ctx | | | Occipital Ctx | | |
| AD 4 Temporal | 11.5 | 25.2 | Control (Path) 1 | 12.4 | 66.4 |
| Ctx | | | Occipital Ctx | | |
| AD 5 Inf | 13.9 | 87.7 | Control (Path) 2 | 19.3 | 12.8 |
| Temporal Ctx | | | Occipital Ctx | | |
| AD 5 Sup | 16.6 | 100.0 | Control (Path) 3 | 0.0 | 9.5 |
| Temporal Ctx | | | Occipital Ctx | | |
| AD 6 Inf | 55.1 | 78.5 | Control (Path) 4 | 27.5 | 12.4 |
| Temporal Ctx | | | Occipital Ctx | | |
| AD 6 Sup | 100.0 | 80.1 | Control 1 | 21.5 | 18.9 |
| Temporal Ctx | | | Parietal Ctx | | |
| Control 1 | 15.6 | 13.1 | Control 2 | 20.0 | 53.6 |
| Temporal Ctx | | | Parietal Ctx | | |
| Control 2 | 24.3 | 42.0 | Control 3 | 0.0 | 17.0 |
| Temporal Ctx | | | Parietal Ctx | | |
| Control 3 | 19.1 | 12.7 | Control (Path) 1 | 20.6 | 49.0 |
| Temporal Ctx | | | Parietal Ctx | | |
| Control 3 | 0.0 | 14.7 | Control (Path) 2 | 5.8 | 30.6 |
| Temporal Ctx | | | Parietal Ctx | | |
| Control (Path) 1 | 15.8 | 58.2 | Control (Path) 3 | 5.8 | 17.0 |
| Temporal Ctx | | | Parietal Ctx | | |
| Control (Path) 2 | 8.3 | 33.0 | Control (Path) 4 | 37.6 | 22.4 |
| Temporal Ctx | | | Parietal Ctx | | |

TABLE BLD
General_screening_panel_v1.4

| | Rel. Exp. (%) Ag3314, Run | | Rel. Exp. (%) Ag3314, Run |
|---|---|---|---|
| Tissue Name | 215678552 | Tissue Name | 215678552 |
| Adipose | 1.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI- | 0.0 |
| | | N87 | |
| Melanoma* M14 | 0.0 | Gastric ca. KATO III | 0.0 |
| Melanoma* LOXIMVI | 0.0 | Colon ca SW-948 | 0.8 |
| Melanoma* SK-MEL-5 | 0.0 | Colon ca. SW480 | 0.0 |
| Squamous cell carcinoma | 0.0 | Colon ca.* (SW480 met) | 0.8 |
| SCC-4 | | SW620 | |
| Testis Pool | 100.0 | Colon ca. HT29 | 0.0 |
| Prostate ca.* (bone met) | 0.0 | Colon ca. HCT-116 | 0.9 |
| PC-3 | | | |
| Prostate Pool | 1.0 | Colon ca. CaCo-2 | 0.0 |
| Placenta | 3.5 | Colon cancer tissue | 0.0 |
| Uterus Pool | 0.7 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 38.2 | Small Intestine Pool | 9.9 |
| Ovarian ca. IGROV-1 | 0.0 | Stomach Pool | 2.3 |
| Ovarian ca. OVCAR-8 | 0.0 | Bone Marrow Pool | 0.8 |
| Ovary | 5.7 | Fetal Heart | 0.9 |
| Breast ca. MCF-7 | 0.0 | Heart Pool | 1.7 |
| Breast ca. MDA-MB-231 | 0.0 | Lymph Node Pool | 0.9 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 1.2 | Thymus Pool | 2.5 |
| Trachea | 1.2 | CNS cancer (glio/astro) U87- | 0.0 |
| | | MG | |
| Lung | 73.7 | CNS cancer (glio/astro) U- | 1.4 |
| | | 118-MG | |
| Fetal Lung | 0.0 | CNS cancer (neuro;met) SK- | 0.0 |
| | | N-AS | |
| Lung ca. NCI-N417 | 0.0 | CNS cancer (astro) SF-539 | 0.0 |
| Lung ca. LX-1 | 1.2 | CNS cancer (astro) SNB-75 | 0.0 |
| Lung ca. NCI-H146 | 0.0 | CNS cancer (glio) SNB-19 | 1.7 |
| Lung ca. SHP-77 | 0.0 | CNS cancer (glio) SF-295 | 0.0 |
| Lung ca. A549 | 0.7 | Brain (Amygdala) Pool | 1.7 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 5.2 |
| Lung ca. HOP-62 | 0.0 | Cerebral Cortex Pool | 2.1 |
| Lung ca. NCI-H522 | 2.0 | Brain (Substantia nigra) Pool | 0.0 |
| Liver | 0.0 | Brain (Thalamus) Pool | 0.0 |
| Fetal Liver | 0.0 | Brain (whole) | 4.5 |
| Liver ca. HepG2 | 0.0 | Spinal Cord Pool | 0.0 |
| Kidney Pool | 25.0 | Adrenal Gland | 0.0 |
| Fetal Kidney | 9.6 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

TABLE BLE
General_screening_panel_v1.5

| | Rel. Exp. (%) Ag4358, Run | | Rel. Exp. (%) Ag4358, Run |
|---|---|---|---|
| Tissue Name | 244373100 | Tissue Name | 244373100 |
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI- | 0.0 |
| | | N87 | |
| Melanoma* M14 | 0.0 | Gastric ca. KATO III | 0.0 |
| Melanoma* LOXIMVI | 0.0 | Colon ca. SW-948 | 2.9 |
| Melanoma* SK-MEL-5 | 0.0 | Colon ca. SW480 | 0.0 |
| Squamous cell carcinoma | 0.0 | Colon ca.* (SW480 met) | 0.0 |
| SCC-4 | | SW620 | |
| Testis Pool | 100.0 | Colon ca. HT29 | 0.0 |
| Prostate ca.* (bone met) | 0.0 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 2.1 | Colon Pool | 0.0 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 1.2 | Fetal Heart | 0.0 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer (glio/astro) U87- | 0.0 |
| | | MG | |
| Lung | 0.0 | CNS cancer (glio/astro) U- | 0.0 |
| | | 118-MG | |
| Fetal Lung | 0.0 | CNS cancer (neuro;met) SK- | 0.0 |
| | | N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) Pool | 0.0 |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 1.5 |

CNS_neurodegeneration_v1.0 Summary: Ag3314/Ag4358 Results from two experiments using different probe/primer sets show moderate agreement. This panel demonstrates the expression of the CG57719-01 gene at low levels in the brains of an independent group of individuals. However, no differential expression of this gene was detected between Alzheimer's diseased postmortem brains and those of non-demented controls in this experiment. The CG57719-01 gene encodes a protein with homology to aspartate aminotransferase.

General_screening_panel_v1.4 Summary: Ag3314 Expression of the CG57719-01 gene is highest in a sample derived from testis (CT=32.2) with low but significant expression also detected in lung and kidney. Thus, the expression of this gene could be used to distinguish these samples from the other samples in the panel. Furthermore, therapeutic modulation of this gene product may be useful for the diagnosis and/or treatment of fertility and hypogonadism.

General_screening_panel_v1.5 Summary: Ag4358 Expression of the CG57719-01 gene is restricted to a sample derived from testis (CT=32.67). This observation is consistent with the results obtained using Ag3314 on Panel 1.4. Thus, the expression of this gene could be used to distinguish testis from the other samples in the panel. Furthermore, therapeutic modulation of this gene product may be useful for the diagnosis and/or treatment of fertility and hypogonadism.

Panel 4.1D Summary: Ag4358 Expression of this gene is low/undetectable (CTs>35) across all of the samples on this panel (data not shown).

Panel 4D Summary: Ag3314 Expression of this gene is low/undetectable (CTs>35) across all of the samples on this panel (data not shown).

NOV70

Expression of NOV70/CG57462-01 was assessed using the primer-probe set Ag3247, described in Table BMA. Results of the RTQ-PCR runs are shown in Tables BMB, BMC, BMD and BME.

TABLE BMA
Probe Name Ag3247

| Primers | Sequences | Length | Start Position | SEQ ID NO |
|---|---|---|---|---|
| Forward | 5′-gacgttcaacaatgacatgct-3′ | 21 | 1940 | 470 |
| Probe | TET-5′-cttcatcagcagcagctgcattgct-3′-TAMRA | 25 | 1967 | 471 |
| Reverse | 5′-agcaggaggtgaggatgtagac-3′ | 22 | 1998 | 472 |

TABLE BMB
CNS_neurodegeneration_v1.0

| | Rel. Exp. (%) Ag3247, Run | | Rel. Exp. (%) Ag3247, Run |
|---|---|---|---|
| Tissue Name | 210037961 | Tissue Name | 210037961 |
| AD 1 Hippo | 18.8 | Control (Path) 3 Temporal | 3.7 |
| | | Ctx | |
| AD 2 Hippo | 28.1 | Control (Path) 4 Temporal | 32.3 |
| | | Ctx | |
| AD 3 Hippo | 3.2 | AD 1 Occipital Ctx | 14.7 |
| AD 4 Hippo | 7.9 | AD 2 Occipital Ctx | 0.0 |
| | | (Missing) | |
| AD 5 Hippo | 97.9 | AD 3 Occipital Ctx | 3.2 |
| AD 6 Hippo | 37.6 | AD 4 Occipital Ctx | 11.5 |
| Control 2 Hippo | 30.1 | AD 5 Occipital Ctx | 15.2 |
| Control 4 Hippo | 8.7 | AD 6 Occipital Ctx | 25.0 |
| Control (Path) 3 Hippo | 3.4 | Control 1 Occipital Ctx | 1.8 |
| AD 1 Temporal Ctx | 17.7 | Control 2 Occipital Ctx | 59.9 |
| AD 2 Temporal Ctx | 41.8 | Control 3 Occipital Ctx | 17.3 |
| AD 3 Temporal Ctx | 6.7 | Control 4 Occipital Ctx | 6.7 |
| AD 4 Temporal Ctx | 27.2 | Control (Path) 1 Occipital | 81.2 |
| | | Ctx | |
| AD 5 Inf Temporal Ctx | 100.0 | Control (Path) 2 Occipital | 10.8 |
| | | Ctx | |
| AD 5 Sup Temporal Ctx | 57.4 | Control (Path) 3 Occipital | 0.6 |
| | | Ctx | |
| AD 6 Inf Temporal Ctx | 23.2 | Control (Path) 4 Occipital | 18.7 |
| | | Ctx | |
| AD 6 Sup Temporal Ctx | 43.5 | Control 1 Parietal Ctx | 7.5 |
| Control 1 Temporal Ctx | 6.0 | Control 2 Parietal Ctx | 57.8 |
| Control 2 Temporal Ctx | 16.3 | Control 3 Parietal Ctx | 14.4 |
| Control 3 Temporal Ctx | 11.7 | Control (Path) 1 Parietal | 37.9 |
| | | Ctx | |
| Control 3 Temporal Ctx | 12.4 | Control (Path) 2 Parietal | 26.1 |
| | | Ctx | |
| Control (Path) 1 Temporal | 61.6 | Control (Path) 3 Parietal | 2.2 |
| Ctx | | Ctx | |
| Control (Path) 2 Temporal | 32.1 | Control (Path) 4 Parietal | 29.7 |
| Ctx | | Ctx | |

TABLE BMC
General_screening_panel_v1.4

| | Rel. Exp. (%) Ag3247, Run | | Rel. Exp. (%) Ag3247, Run |
|---|---|---|---|
| Tissue Name | 214693633 | Tissue Name | 214693633 |
| Adipose | 0.1 | Renal ca. TK-10 | 1.9 |
| Melanoma* Hs688(A).T | 0.1 | Bladder | 0.5 |
| Melanoma* Hs688(B). T | 0.0 | Gastric ca. (liver met.) NCI- | 0.0 |
| | | N87 | |
| Melanoma* M14 | 0.1 | Gastric ca. KATO III | 0.0 |
| Melanoma* LOXIMVI | 0.1 | Colon ca. SW-948 | 0.0 |
| Melanoma* SK-MEL-5 | 0.0 | Colon ca. SW480 | 1.2 |
| Squamous cell carcinoma | 0.5 | Colon ca.* (SW480 met) | 1.1 |
| SCC-4 | | SW620 | |
| Testis Pool | 2.8 | Colon ca. HT29 | 0.3 |
| Prostate ca.* (bone met) | 0.7 | Colon ca. HCT-116 | 1.6 |
| PC-3 | | | |
| Prostate Pool | 0.3 | Colon ca. CaCo-2 | 0.9 |
| Placenta | 0.0 | Colon cancer tissue | 0.0 |
| Uterus Pool | 0.0 | Colon ca. SW1116 | 1.2 |
| Ovarian ca. OVCAR-3 | 0.3 | Colon ca. Colo-205 | 0.0 |
| Ovarian ca. SK-OV-3 | 0.1 | Colon ca. SW-48 | 0.1 |
| Ovarian ca. OVCAR-4 | 0.0 | Colon Pool | 0.2 |
| Ovarian ca. OVCAR-5 | 0.4 | Small Intestine Pool | 0.1 |
| Ovarian ca. IGROV-1 | 0.1 | Stomach Pool | 0.4 |
| Ovarian ca. OVCAR-8 | 0.1 | Bone Marrow Pool | 0.0 |
| Ovary | 0.1 | Fetal Heart | 0.0 |
| Breast ca. MCF-7 | 4.2 | Heart Pool | 0.0 |
| Breast ca. MDA-MB-231 | 0.2 | Lymph Node Pool | 0.1 |
| Breast ca. BT 549 | 0.4 | Fetal Skeletal Muscle | 0.1 |
| Breast ca. T47D | 0.9 | Skeletal Muscle Pool | 0.0 |
| Breast ca. MDA-N | 0.4 | Spleen Pool | 0.1 |
| Breast Pool | 0.4 | Thymus Pool | 0.6 |
| Trachea | 0.0 | CNS cancer (glio/astro) U87- | 0.0 |
| | | MG | |
| Lung | 0.1 | CNS cancer (glio/astro) U- | 0.0 |
| | | 118-MG | |
| Fetal Lung | 0.5 | CNS cancer (neuro;met) SK- | 0.2 |
| | | N-AS | |
| Lung ca. NCI-N417 | 8.7 | CNS cancer (astro) SF-539 | 0.1 |
| Lung ca. LX-1 | 0.2 | CNS cancer (astro) SNB-75 | 0.2 |
| Lung ca. NCI-H146 | 1.6 | CNS cancer (glio) SNB-19 | 0.0 |
| Lung ca. SHP-77 | 6.3 | CNS cancer (glio) SF-295 | 0.4 |
| Lung ca. A549 | 0.0 | Brain (Amygdala) Pool | 3.6 |
| Lung ca. NCI-H526 | 1.2 | Brain (cerebellum) | 16.7 |
| Lung ca. NCI-H23 | 4.5 | Brain (fetal) | 100.0 |
| Lung ca. NCI-H460 | 0.4 | Brain (Hippocampus) Pool | 6.0 |
| Lung ca. HOP-62 | 0.0 | Cerebral Cortex Pool | 6.0 |
| Lung ca. NCI-H522 | 2.2 | Brain (Substantia nigra) Pool | 5.6 |
| Liver | 0.0 | Brain (Thalamus) Pool | 6.8 |
| Fetal Liver | 0.1 | Brain (whole) | 12.8 |
| Liver ca. HepG2 | 0.4 | Spinal Cord Pool | 3.8 |
| Kidney Pool | 0.0 | Adrenal Gland | 0.4 |
| Fetal Kidney | 1.6 | Pituitary gland Pool | 0.7 |
| Renal ca. 786-0 | 0.7 | Salivary Gland | 0.0 |
| Renal ca. A498 | 0.2 | Thyroid (female) | 0.0 |
| Renal ca. ACHN | 0.1 | Pancreatic ca. CAPAN2 | 1.2 |
| Renal ca. UO-31 | 0.5 | Pancreas Pool | 0.5 |

TABLE BMD
Panel 2.2

| | Rel. Exp. (%) Ag3247, Run | | Rel. Exp. (%) Ag3247, Run |
|---|---|---|---|
| Tissue Name | 174441297 | Tissue Name | 174441297 |
| Normal Colon | 0.7 | Kidney Margin (OD04348) | 2.4 |
| Colon cancer (OD06064) | 0.0 | Kidney malignant cancer | 0.0 |
| | | (OD06204B) | |
| Colon Margin (OD06064) | 0.0 | Kidney normal adjacent tissue | 0.0 |
| | | (OD06204E) | |
| Colon cancer (OD06159) | 0.0 | Kidney Cancer (OD04450-01) | 3.4 |
| Colon Margin (OD06159) | 0.0 | Kidney Margin (OD04450-03) | 2.2 |
| Colon cancer (OD06297-04) | 0.0 | Kidney Cancer 8120613 | 0.0 |
| Colon Margin (OD06297-05) | 0.0 | Kidney Margin 8120614 | 0.0 |
| CC Gr.2 ascend colon | 1.4 | Kidney Cancer 9010320 | 0.0 |
| (ODO3921) | | | |
| CC Margin (ODO3921) | 0.0 | Kidney Margin 9010321 | 0.0 |
| Colon cancer metastasis | 0.0 | Kidney Cancer 8120607 | 2.5 |
| (OD06104) | | | |
| Lung Margin (OD06104) | 0.0 | Kidney Margin 8120608 | 1.2 |
| Colon mets to lung (OD04451- | 4.1 | Normal Uterus | 1.2 |
| 01) | | | |
| Lung Margin (OD04451-02) | 0.0 | Uterine Cancer 064011 | 2.7 |
| Normal Prostate | 3.3 | Normal Thyroid | 0.0 |
| Prostate Cancer (OD04410) | 0.0 | Thyroid Cancer 064010 | 0.0 |
| Prostate Margin (OD04410) | 0.0 | Thyroid Cancer A302152 | 0.0 |
| Normal Ovary | 8.0 | Thyroid Margin A302153 | 0.0 |
| Ovarian cancer (OD06283-03) | 0.0 | Normal Breast | 2.4 |
| Ovarian Margin (OD06283-07) | 0.0 | Breast Cancer (OD04566) | 0.0 |
| Ovarian Cancer 064008 | 4.2 | Breast Cancer 1024 | 1.3 |
| Ovarian cancer (OD06145) | 0.0 | Breast Cancer (OD04590-01) | 7.1 |
| Ovarian Margin (OD06145) | 0.0 | Breast Cancer Mets (OD04590- | 4.4 |
| | | 03) | |
| Ovarian cancer (OD06455-03) | 0.0 | Breast Cancer Metastasis | 100.0 |
| | | (OD04655-05) | |
| Ovarian Margin (OD06455-07) | 0.0 | Breast Cancer 064006 | 1.2 |
| Normal Lung | 1.6 | Breast Cancer 9100266 | 2.5 |
| Invasive poor diff. lung adeno | 0.0 | Breast Margin 9100265 | 0.0 |
| (ODO4945-01) | | | |
| Lung Margin (ODO4945-03) | 0.0 | Breast Cancer A209073 | 3.4 |
| Lung Malignant Cancer | 0.0 | Breast Margin A2090734 | 0.0 |
| (OD03126) | | | |
| Lung Margin (OD03126) | 0.0 | Breast cancer (OD06083) | 3.2 |
| Lung Cancer (OD05014A) | 0.0 | Breast cancer node metastasis | 2.5 |
| | | (OD06083) | |
| Lung Margin (OD05014B) | 0.6 | Normal Liver | 0.0 |
| Lung cancer (OD06081) | 0.0 | Liver Cancer 1026 | 0.0 |
| Lung Margin (OD06081) | 1.4 | Liver Cancer 1025 | 2.1 |
| Lung Cancer (OD04237-01) | 0.0 | Liver Cancer 6004-T | 0.0 |
| Lung Margin (OD04237-02) | 0.4 | Liver Tissue 6004-N | 0.0 |
| Ocular Melanoma Metastasis | 0.0 | Liver Cancer 6005-T | 2.4 |
| Ocular Melanoma Margin | 0.0 | Liver Tissue 6005-N | 0.0 |
| (Liver) | | | |
| Melanoma Metastasis | 0.0 | Liver Cancer 064003 | 0.0 |
| Melanoma Margin (Lung) | 0.0 | Normal Bladder | 0.0 |
| Normal Kidney | 0.0 | Bladder Cancer 1023 | 0.0 |
| Kidney Ca, Nuclear grade 2 | 0.0 | Bladder Cancer A302173 | 0.0 |
| (OD04338) | | | |
| Kidney Margin (OD04338) | 0.0 | Normal Stomach | 0.0 |
| Kidney Ca Nuclear grade 1/2 | 1.2 | Gastric Cancer 9060397 | 0.8 |
| (OD04339) | | | |
| Kidney Margin (OD04339) | 0.0 | Stomach Margin 9060396 | 0.9 |
| Kidney Ca, Clear cell type | 0.0 | Gastric Cancer 9060395 | 0.0 |
| (OD04340) | | | |
| Kidney Margin (OD04340) | 0.0 | Stomach Margin 9060394 | 1.4 |
| Kidney Ca, Nuclear grade 3 | 0.0 | Gastric Cancer 064005 | 0.0 |
| (OD04348) | | | |

TABLE BME
Panel 4D

| | Rel. Exp. (%) Ag3247, Run | | Rel. Exp. (%) Ag3247, Run |
|---|---|---|---|
| Tissue Name | 164390951 | Tissue Name | 164390951 |
| Secondary Th1 act | 1.6 | HUVEC IL-1beta | 0.6 |
| Secondary Th2 act | 0.6 | HUVEC IFN gamma | 4.1 |
| Secondary Tr1 act | 0.8 | HUVEC TNF alpha + IFN gamma | 0.0 |
| Secondary Th1 rest | 0.0 | HUVEC TNF alpha + IL4 | 1.2 |
| Secondary Th2 rest | 0.8 | HUVEC IL-11 | 3.1 |
| Secondary Tr1 rest | 0.0 | Lung Microvascular EC none | 17.1 |
| Primary Th1 act | 7.0 | Lung Microvascular EC TNF alpha + | 11.0 |
| | | IL-1beta | |
| Primary Th2 act | 3.5 | Microvascular Dermal EC none | 0.6 |
| Primary Tr1 act | 4.9 | Microsvasular Dermal EC | 0.7 |
| | | TNF alpha + IL-1beta | |
| Primary Th1 rest | 0.6 | Bronchial epithelium TNF alpha + | 100.0 |
| | | IL1beta | |
| Primary Th2 rest | 1.2 | Small airway epithelium none | 1.8 |
| Primary Tr1 rest | 4.7 | Small airway epithelium TNF alpha + | 28.3 |
| | | IL-1beta | |
| CD45RA CD4 lymphocyte | 0.7 | Coronery artery SMC rest | 0.0 |
| act | | | |
| CD45RO CD4 lymphocyte | 2.7 | Coronery artery SMC TNF alpha + | 0.0 |
| act | | IL-1beta | |
| CD8 lymphocyte act | 3.0 | Astrocytes rest | 1.2 |
| Secondary CD8 lymphocyte | 4.2 | Astrocytes TNF alpha + IL-1beta | 0.6 |
| rest | | | |
| Secondary CD8 lymphocyte | 0.7 | KU-812 (Basophil) rest | 0.0 |
| act | | | |
| CD4 lymphocyte none | 1.3 | KU-812 (Basophil) | 0.7 |
| | | PMA/ionomycin | |
| 2ry Th1/Th2/Tr1_anti-CD95 | 0.7 | CCD1106 (Keratinocytes) none | 37.1 |
| CH11 | | | |
| LAK cells rest | 0.0 | CCD1106 (Keratinocytes) | 28.9 |
| | | TNF alpha + IL-1beta | |
| LAK cells IL-2 | 4.0 | Liver cirrhosis | 6.2 |
| LAK cells IL-2 + IL-12 | 2.1 | Lupus kidney | 1.1 |
| LAK cells IL-2 + IFN gamma | 6.5 | NCI-H292 none | 2.5 |
| LAK cells IL-2 + IL-18 | 4.7 | NCI-H292 IL-4 | 1.3 |
| LAK cells PMA/ionomycin | 0.5 | NCI-H292 IL-9 | 4.6 |
| NK Cells IL-2 rest | 1.3 | NCI-H292 IL-13 | 1.1 |
| Two Way MLR 3 day | 1.8 | NCI-H292 IFN gamma | 0.0 |
| Two Way MLR 5 day | 0.5 | HPAEC none | 0.0 |
| Two Way MLR 7 day | 1.7 | HPAEC TNF alpha + IL-1beta | 0.0 |
| PBMC rest | 0.0 | Lung fibroblast none | 0.0 |
| PBMC PWM | 6.0 | Lung fibroblast TNF alpha + IL-1 | 0.0 |
| | | beta | |
| PBMC PHA-L | 0.6 | Lung fibroblast IL-4 | 0.0 |
| Ramos (B cell) none | 1.9 | Lung fibroblast IL-9 | 0.0 |
| Ramos (B cell) ionomycin | 1.9 | Lung fibroblast IL-13 | 0.0 |
| B lymphocytes PWM | 1.3 | Lung fibroblast IFN gamma | 0.0 |
| B lymphocytes CD40L and | 5.0 | Dermal fibroblast CCD1070 rest | 0.7 |
| IL-4 | | | |
| EOL-1 dbcAMP | 3.0 | Dermal fibroblast CCD1070 TNF | 0.7 |
| | | alpha | |
| EOL-1 dbcAMP | 0.6 | Dermal fibroblast CCD1070 IL-1 | 0.0 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 0.7 |
| Dendritic cells anti-CD40 | 0.0 | IBD Colitis 2 | 0.0 |
| Monocytes rest | 0.6 | IBD Crohn's | 0.0 |
| Monocytes LPS | 0.0 | Colon | 2.7 |
| Macrophages rest | 0.0 | Lung | 0.6 |
| Macrophages LPS | 0.0 | Thymus | 1.0 |
| HUVEC none | 0.0 | Kidney | 7.0 |
| HUVEC starved | 5.0 | | |

CNS_neurodegeneration_v1.0 Summary: Ag3247 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3247 Highest expression of CG57462-01 is seen in samples derived from fetal brain (CT=26.5). In addition moderate expression of this gene is also observed in samples derived from whole brain, thalamus, Substantia nigra, cerebral cortex and hippocampus. Thus, expression of this gene could be used to differentiate between these samples and other samples on this panel. Also, this gene may play a role in central nervous system disorders such as Alzheimer's disease, Parkinson's disease, epilepsy, multiple sclerosis, schizophrenia and depression.

The CG57462-01 gene encodes for a protein containing a patched domain (IPR003392). Patched, is a receptor for the morphogene sonic hedgehog. In Drosophila melanogaster, this protein associates with the smoothened protein to transduce hedgehog signals, leading to the activation of wingless, decapentaplegic and patched itself. It participates in cell interactions that establish pattern within the segment and imaginal disks during development. In mammals, the Hedgehog (HH) signaling pathway is involved in patterning and development of a variety of organ systems, including the nervous system, the skeletal system, the craniofacial structures, and the gastrointestinal tract. Abnormalities in this signaling cascade have been found in various developmental pathologies and neoplasms such as basal cell carcinoma, Niemann-Pick type II disease, holoprosencephaly. The abnormalities are associated with congenital or sporadic genetic alteration affecting function of different components of the HH signaling pathway, including SHH, PTCH, SMOH and GLI proteins (Ref. 2, 3). Thus, therapeutic modulation of the activity of the protein encoded by the CG57463-01 gene may be useful in the treatment of basal cell carcinoma, Niemann-Pick type II disease, and holoprosencephaly

References:

1. IPR003392: Patched family

The transmembrane protein, patched, is a receptor for the morphogene Sonic Hedgehog. In Drosophila melanogaster, this protein associates with the smoothened protein to transduce hedgehog signals, leading to the activation of wingless, decapentaplegic and patched itself. It participates in cell interactions that establish pattern within the segment and imaginal disks during development. The mouse homolog may play a role in epidermal development. The human Niemann-Pick C1 protein, defects in which cause Niemann-Pick type II disease, is also a member of this family. This protein is involved in the intracellular trafficking of cholesterol, and may play a role in vesicular trafficking in glia, a process that may be crucial for maintaining the structural functional integrity of nerve terminals.

2. Incardona J P, Roelink H. (2000) The role of cholesterol in Shh signaling and teratogen-induced holoprosencephaly. Cell Mol Life Sci 57(12):1709-19.

Holoprosencephaly, or an undivided forebrain, is a complex brain malformation associated with Sonic hedgehog (Shh) mutations. Other causes of holoprosencephaly have converged upon the Shh signaling pathway: genetic and pharmacologic impairment of cholesterol synthesis, and the action of the steroidal alkaloid cyclopamine. This review focuses on recent studies aimed at determining how Shh signaling is affected by these causes of holoprosencephaly, whether they involve a common mechanism and the role played by cholesterol. Cholesterol is potentially important for both biogenesis of Shh and in signal transduction in Shh-responsive cells. Teratogens that induce holoprosencephaly appear to affect Shh signal transduction rather than Shh biogenesis. Analysis of these agents and other compounds that affect various aspects of cellular cholesterol distribution indicates that the role of cholesterol in Shh signal transduction is novel and complicated. The similarity of the Shh receptor, Patched (Ptc), to the Niemann-Pick Cl protein, which is involved in the vesicular trafficking of cholesterol, provides insight into the role of cholesterol and the action of compounds like cyclopamine.

PMID: 11130177

3. Oldak M, Grzela T, Lazarczyk M, Malejczyk J, Skopinski P. (2001) Clinical aspects of disrupted Hedgehog signaling (Review). Int J Mol Med 8(4):445-52.

The Hedgehog (HH) signaling pathway is involved in patterning and development of a variety of organ systems, including the nervous system, the skeletal system, the craniofacial structures, and the gastrointestinal tract. Recent evidence also implicates this signaling pathway in the postembryonic regulation of stem-cell number in epithelia and blood. The family of HH proteins consists of at least three different members, i.e., sonic HH(SHH), Indian HH (IHH), and desert HH (DHH). SHH is the most broadly expressed member of this family and is probably responsible for the major effects of this signaling pathway. The HH signal is received and transduced via a specific receptor complex composed of patched (PTCH) and smoothened (SMOH) transmembrane proteins. Abnormalities in this signaling cascade have been found in various developmental pathologies and neoplasms such as basal cell carcinoma. The abnormalities are associated with congenital or sporadic genetic alteration affecting function of different components of the HH signaling pathway, including SHH, PTCH, SMOH and GLI proteins.

PMID: 11562786

Panel 2.2 Summary: Ag3247 Expression of the CG57462-01 gene is highest in a metastatic breast cancer sample (CT=29.8). Strikingly, this gene is expressed at higher levels in breast tumors when compared to their matched normal breast controls. Thus, expression of this gene may be used for the diagnosis of breast cancer. Furthermore, therapeutic modulation of the activity of this gene or its protein product, using small molecule drugs, antibodies or protein therapeutics, may be of benefit in the treatment of breast cancer.

Panel 4D Summary: Ag3247 Expression of this gene is highest in bronchial epithelium treated with TNF alpha and IL-1 beta (CT=30). In addition, expression of this gene is upregulated in activated small airway epithelium. Thus, therapeutic modulation of the activity of this gene may be of benefit in the treatment of asthma and emphysema.

NOV71

Expression of NOV71/CG57584-01 was assessed using the primer-probe set Ag3290, described in Table BNA. Results of the RTQ-PCR runs are shown in Tables BNB, and BNC.

TABLE BNA
Probe Name Ag3290

| Primers | Sequences | Length | Start Position | SEQ ID NO |
|---|---|---|---|---|
| Forward | 5′-ccctcagaggactggtttactt-3′ | 22 | 1598 | 473 |
| Probe | TET-5′-cacctctgcctgccacacctcag-3′-TAMRA | 23 | 1629 | 474 |
| Reverse | 5′-ctacatgcagtggagcaagtct-3′ | 22 | 1658 | 475 |

TABLE BNB
CNS_neurodegeneration_v1.0

| | Rel. Exp. (%) Ag3290, Run | | Rel. Exp. (%) Ag3290, Run |
|---|---|---|---|
| Tissue Name | 210062016 | Tissue Name | 210062016 |
| AD 1 Hippo | 24.8 | Control (Path) 3 Temporal | 11.5 |
| | | Ctx | |
| AD 2 Hippo | 15.1 | Control (Path) 4 Temporal | 94.0 |
| | | Ctx | |
| AD 3 Hippo | 0.0 | AD 1 Occipital Ctx | 12.6 |
| AD 4 Hippo | 19.1 | AD 2 Occipital Ctx | 0.0 |
| | | (Missing) | |
| AD 5 hippo | 51.8 | AD 3 Occipital Ctx | 33.9 |
| AD 6 Hippo | 28.7 | AD 4 Occipital Ctx | 0.0 |
| Control 2 Hippo | 26.8 | AD 5 Occipital Ctx | 0.0 |
| Control 4 Hippo | 23.8 | AD 6 Occipital Ctx | 55.1 |
| Control (Path) 3 Hippo | 6.3 | Control 1 Occipital Ctx | 17.4 |
| AD 1 Temporal Ctx | 12.2 | Control 2 Occipital Ctx | 100.0 |
| AD 2 Temporal Ctx | 42.0 | Control 3 Occipital Ctx | 29.3 |
| AD 3 Temporal Ctx | 9.8 | Control 4 Occipital Ctx | 21.0 |
| AD 4 Temporal Ctx | 62.0 | Control (Path) 1 Occipital | 52.9 |
| | | Ctx | |
| AD 5 Inf Temporal Ctx | 14.0 | Control (Path) 2 Occipital | 15.4 |
| | | Ctx | |
| AD 5 SupTemporal Ctx | 26.6 | Control (Path) 3 Occipital | 9.8 |
| | | Ctx | |
| AD 6 Inf Temporal Ctx | 12.5 | Control (Path) 4 Occipital | 13.0 |
| | | Ctx | |
| AD 6 Sup Temporal Ctx | 2.3 | Control 1 Parietal Ctx | 13.8 |
| Control 1 Temporal Ctx | 39.0 | Control 2 Parietal Ctx | 76.3 |
| Control 2 Temporal Ctx | 39.8 | Control 3 Parietal Ctx | 21.3 |
| Control 3 Temporal Ctx | 0.0 | Control (Path) 1 Parietal | 50.3 |
| | | Ctx | |
| Control 4 Temporal Ctx | 12.4 | Control (Path) 2 Parietal | 16.0 |
| | | Ctx | |
| Control (Path) 1 Temporal | 48.3 | Control (Path) 3 Parietal | 19.8 |
| Ctx | | Ctx | |
| Control (Path) 2 Temporal | 51.1 | Control (Path) 4 Parietal | 84.7 |
| Ctx | | Ctx | |

TABLE BNC
Panel 4D

| | Rel. Exp. (%) Ag3290, Run | | Rel. Exp. (%) Ag3290, Run |
|---|---|---|---|
| Tissue Name | 164331693 | Tissue Name | 164331693 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 14.9 | HUVEC IL-11 | 0.0 |
| Secondary Tr1 rest | 3.6 | Lung Microvascular EC none | 0.0 |
| Primary Th1 act | 0.0 | Lung Microvascular EC TNF alpha + | 0.0 |
| | | IL-1beta | |
| Primary Th2 act | 0.0 | Microvascular Dermal EC none | 0.0 |
| Primary Tr1 act | 4.0 | Microsvasular Dermal EC | 0.0 |
| | | TNF alpha + IL-1beta | |
| Primary Th1 rest | 9.4 | Bronchial epithelium TNF alpha + | 0.0 |
| | | IL1beta | |
| Primary Th2 rest | 36.6 | Small airway epithelium none | 9.1 |
| Primary Tr1 rest | 16.2 | Small airway epithelium TNF alpha + | 0.0 |
| | | IL-1beta | |
| CD45RA CD4 lymphocyte | 0.0 | Coronery artery SMC rest | 100.0 |
| act | | | |
| CD45RO CD4 lymphocyte | 0.0 | Coronery artery SMC TNF alpha + | 38.2 |
| act | | IL-1beta | |
| CD8 lymphocyte act | 0.0 | Astrocytes rest | 4.0 |
| Secondary CD8 lymphocyte | 0.0 | Astrocytes TNF alpha + IL-1beta | 3.7 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 0.5 |
| | | PMA/ionomycin | |
| 2ry Th1/Th2/Tr1_anti-CD95 | 6.8 | CCD1106 (Keratinocytes) none | 0.0 |
| CH11 | | | |
| LAK cells rest | 0.0 | CCD1106 (Keratinocytes) | 0.0 |
| | | TNF alpha + IL-1beta | |
| LAK cells IL-2 | 2.9 | Liver cirrhosis | 0.0 |
| LAK cells IL-2 + IL-12 | 0.0 | Lupus kidney | 0.0 |
| LAK cells IL-2 + IFN gamma | 4.6 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.5 |
| NK Cells IL-2 rest | 0.0 | NCI-H292 IL-13 | 4.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 12.8 | Lung fibroblast none | 0.0 |
| PBMC PWM | 0.0 | Lung fibroblast TNF alpha + IL-1 | 0.0 |
| | | beta | |
| PBMC PHA-L | 4.4 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 0.0 | Dermal fibroblast CCD1070 rest | 17.6 |
| IL-4 | | | |
| EOL-1 dbcAMP | 0.0 | Dermal fibroblast CCD1070 TNF | 13.9 |
| | | alpha | |
| EOL-1 dbcAMP | 0.0 | Dermal fibroblast CCD1070 IL-1 | 8.9 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 4.2 | Colon | 3.6 |
| Macrophages rest | 3.7 | Lung | 16.2 |
| Macrophages LPS | 0.0 | Thymus | 0.0 |
| HUVEC none | 0.0 | Kidney | 26.1 |
| HUVEC starved | 0.0 | | |

CNS_neurodegeneration_v1.0 Summary: Ag3290 This panel demonstrates the expression of this gene at low levels in the brains of several individuals. However, no differential expression of this gene was detected between Alzheimer's diseased postmortem brains and those of non-demented controls in this experiment. The CG57584-01 gene encodes a protein with homology to zona pellucida sperm-binding protein 1 precursor.

General_screening_panel_v1.4 Summary: Ag3290 Results from one experiment with the CG57584-01 gene are not included. The amp plot indicates that there were experimental difficulties with this run.

Panel 4D Summary: Ag3290 Expression of the CG57584-01 gene is highest in resting coronary artery smooth muscle cells (CT=31.6). In addition, low but significant expression of this gene is seen in normal lung and kidney samples as well as in dermal fibroblasts. Thus, expression of this gene may be used to distinguish these samples from the other samples on this panel. Furthermore, therapeutic modulation of the activity of this gene may be useful for the treatment of asthma and restenosis.

NOV72

Expression of NOV72/CG56761-01 was assessed using the primer-probe sets Ag3011, Ag1810 and Ag1811, described in Tables BOA, BOB and BOC. Results of the RTQ-PCR runs are shown in Tables BOD, BOE, BOF, BOG, BOH and BOI.

TABLE BOA
Probe Name Ag3011

| Primers | Sequences | Length | Start Position | Seq id no: |
|---|---|---|---|---|
| Forward | 5′-gaactgttccttgtggtttcc-3′ | 21 | 4438 | 476 |
| Probe | TET-5′-accacaggcatcagcagtcccatt-3′-TAMRA | 24 | 4470 | 477 |
| Reverse | 5′-acactttgcctcaggtgactt-3′ | 21 | 4495 | 478 |

TABLE BOB
Probe Name Ag1810

| Primers | Sequences | Length | Start Position | Seq id no: |
|---|---|---|---|---|
| Forward | 5′-gtcaggagaactgttccttgtg-3′ | 22 | 4431 | 479 |
| Probe | TET-5′-accacaggcatcagcagtcccat-3′-TAMRA | 23 | 4470 | 480 |
| Reverse | 5′-ctttgcctcaggtgacttga-3′ | 20 | 4493 | 481 |

TABLE BOC
Probe Name Ag1811

| Primers | Sequences | Length | Start Position | Seq id no: |
|---|---|---|---|---|
| Forward | 5′-ttggagcctttgctcttatgt-3′ | 21 | 5503 | 482 |
| Probe | TET-5′-aggggagaccatccccaagttacaaa-3′-TAMRA | 26 | 5467 | 483 |
| Reverse | 5′-aagatgcccagaaaagctgta-3′ | 21 | 5432 | 484 |

TABLE BOD
A1_comprehensive panel_v1.0

| | Rel. Exp. (%) Ag3011, Run | | Rel. Exp. (%) Ag3011, Run |
|---|---|---|---|
| Tissue Name | 225567597 | Tissue Name | 225567597 |
| 110967 COPD-F | 6.1 | 112427 Match Control | 35.6 |
| | | Psoriasis-F | |
| 110980 COPD-F | 11.8 | 112418 Psoriasis-M | 10.7 |
| 110968 COPD-M | 7.4 | 112723 Match Control | 0.1 |
| | | Psoriasis-M | |
| 110977 COPD-M | 0.0 | 112419 Psoriasis-M | 11.4 |
| 110989 Emphysema-F | 8.0 | 112424 Match Control | 3.6 |
| | | Psoriasis-M | |
| 110992 Emphysema-F | 3.8 | 112420 Psoriasis-M | 12.2 |
| 110993 Emphysema-F | 6.3 | 112425 Match Control | 25.5 |
| | | Psoriasis-M | |
| 110994 Emphysema-F | 2.4 | 104689 (ME) OA Bone-Backus | 24.5 |
| 110995 Emphysema-F | 8.5 | 104690 (ME) Adj “Normal” | 24.0 |
| | | Bone-Backus | |
| 110996 Emphysema-F | 1.0 | 104691 (ME) OA Synovium- | 10.6 |
| | | Backus | |
| 110997 Asthma-M | 2.3 | 104692 (BA) OA Cartilage- | 0.0 |
| | | Backus | |
| 111001 Asthma-F | 6.7 | 104694 (BA) OA Bone-Backus | 8.5 |
| 111002 Asthma-F | 5.5 | 104695 (BA) Adj “Normal” | 27.2 |
| | | Bone-Backus | |
| 111003 Atopic Asthma-F | 5.0 | 104696 (BA) OA Synovium- | 6.4 |
| | | Backus | |
| 111004 Atopic Asthma-F | 2.5 | 104700 (SS) OA Bone-Backus | 14.6 |
| 111005 Atopic Asthma-F | 1.8 | 104701 (SS) Adj “Normal” | 22.8 |
| | | Bone-Backus | |
| 111006 Atopic Asthma-F | 0.4 | 104702 (SS) OA Synovium- | 23.8 |
| | | Backus | |
| 111417 Allergy-M | 2.4 | 117093 OA Cartilage Rep7 | 5.8 |
| 112347 Allergy-M | 0.2 | 112672 OA Bone5 | 10.9 |
| 112349 Normal Lung-F | 0.7 | 112673 OA Synovium5 | 4.6 |
| 112357 Normal Lung-F | 4.6 | 112674 OA Synovial Fluid | 5.6 |
| | | cells5 | |
| 112354 Normal Lung-M | 5.8 | 117100 OA Cartilage Rep14 | 5.3 |
| 112374 Crohns-F | 1.3 | 112756 OA Bone9 | 20.3 |
| 112389 Match Control | 4.2 | 112757 OA Synovium9 | 2.8 |
| Crohns-F | | | |
| 112375 Crohns-F | 1.0 | 112758 OA Synovial Fluid | 4.1 |
| | | Cells9 | |
| 112732 Match Control | 56.6 | 117125 RA Cartilage Rep2 | 12.4 |
| Crohns-F | | | |
| 112725 Crohns-M | 0.5 | 113492 Bone2 RA | 21.2 |
| 112387 Match Control | 4.9 | 113493 Synovium2 RA | 9.4 |
| Crohns-M | | | |
| 112378 Crohns-M | 1.0 | 113494 Syn Fluid Cells RA | 12.8 |
| 112390 Match Control | 7.9 | 113499 Cartilage4 RA | 12.8 |
| Crohns-M | | | |
| 112726 Crohns-M | 5.0 | 113500 Bone4 RA | 19.5 |
| 112731 Match Control | 5.9 | 113501 Synovium4 RA | 14.0 |
| Crohns-M | | | |
| 112380 Ulcer Col-F | 5.2 | 113502 Syn Fluid Cells4 RA | 6.4 |
| 112734 Match Control Ulcer | 100.0 | 113495 Cartilage3 RA | 11.5 |
| Col-F | | | |
| 112384 Ulcer Col-F | 11.0 | 113496 Bone3 RA | 13.8 |
| 112737 Match Control Ulcer | 1.7 | 113497 Synovium3 RA | 5.6 |
| Col-F | | | |
| 112386 Ulcer Col-F | 4.5 | 113498 Syn Fluid Cells3 RA | 15.3 |
| 112738 Match Control Ulcer | 5.3 | 117106 Normal Cartilage | 5.9 |
| Col-F | | Rep20 | |
| 112381 Ulcer Col-M | 3.1 | 113663 Bone3 Normal | 0.4 |
| 112735 Match Control Ulcer | 2.3 | 113664 Synovium3 Normal | 0.0 |
| Col-M | | | |
| 112382 Ulcer Col-M | 6.7 | 113665 Syn Fluid Cells3 | 0.0 |
| | | Normal | |
| 112394 Match Control Ulcer | 2.4 | 117107 Normal Cartilage | 3.0 |
| Col-M | | Rep22 | |
| 112383 Ulcer Col-M | 6.6 | 113667 Bone4 Normal | 2.8 |
| 112736 Match Control Ulcer | 3.0 | 113668 Synovium4 Normal | 3.4 |
| Col-M | | | |
| 112423 Psoriasis-F | 4.2 | 113669 Syn Fluid Cells4 | 4.3 |
| | | Normal | |

TABLE BOE
CNS_neurodegeneration_v1.0

| | Rel. Exp.(%) | Rel. Exp.(%) | Rel. Exp.(%) | | Rel. Exp.(%) | Rel. Exp.(%) | Rel. Exp.(%) |
|---|---|---|---|---|---|---|---|
| Tissue | Ag1810, Run | Ag1811, Run | Ag3011, Run | Tissue | Ag1810, Run | Ag1811, Run | Ag3011, Run |
| Name | 207742479 | 207776005 | 211010257 | Name | 207742479 | 207776005 | 211010257 |
| AD 1 Hippo | 7.8 | 11.3 | 9.1 | Control | 4.3 | 6.9 | 4.0 |
| | | | | (Path) 3 | | | |
| | | | | Temporal | | | |
| | | | | Ctx | | | |
| AD 2 Hippo | 13.1 | 21.9 | 15.2 | Control | 20.7 | 26.6 | 23.5 |
| | | | | (Path) 4 | | | |
| | | | | Temporal | | | |
| | | | | Ctx | | | |
| AD 3 Hippo | 4.6 | 6.7 | 5.3 | AD 1 | 12.7 | 21.3 | 13.3 |
| | | | | Occipital | | | |
| | | | | Ctx | | | |
| AD 4 Hippo | 4.6 | 6.2 | 3.7 | AD 2 | 0.2 | 0.0 | 0.0 |
| | | | | Occipital | | | |
| | | | | Ctx | | | |
| | | | | (Missing) | | | |
| AD 5 Hippo | 100.0 | 100.0 | 100.0 | AD 3 | 6.1 | 10.9 | 6.9 |
| | | | | Occipital | | | |
| | | | | Ctx | | | |
| AD 6 Hippo | 19.9 | 19.9 | 18.8 | AD 4 | 12.1 | 16.7 | 13.4 |
| | | | | Occipital | | | |
| | | | | Ctx | | | |
| Control 2 | 11.9 | 19.1 | 12.3 | AD 5 | 29.1 | 41.2 | 21.5 |
| Hippo | | | | Occipital | | | |
| | | | | Ctx | | | |
| Control 4 | 3.9 | 7.0 | 3.4 | AD 6 | 17.3 | 17.4 | 31.6 |
| Hippo | | | | Occipital | | | |
| | | | | Ctx | | | |
| Control | 3.7 | 5.0 | 3.6 | Control 1 | 3.7 | 5.7 | 3.4 |
| (Path) 3 | | | | Occipital | | | |
| Hippo | | | | Ctx | | | |
| AD 1 | 11.7 | 15.0 | 9.5 | Control 2 | 59.9 | 81.2 | 62.0 |
| Temporal | | | | Occipital | | | |
| Ctx | | | | Ctx | | | |
| AD 2 | 20.9 | 22.7 | 17.7 | Control 3 | 13.9 | 81.2 | 62.0 |
| Temporal | | | | Occipital | | | |
| Ctx | | | | Ctx | | | |
| AD 3 | 5.8 | 9.0 | 5.4 | Control 4 | 3.7 | 8.8 | 3.8 |
| Temporal | | | | Occipital | | | |
| Ctx | | | | Ctx | | | |
| AD 4 | 15.2 | 17.8 | 14.1 | Control | 51.4 | 58.6 | 54.7 |
| Temporal | | | | (Path) 1 | | | |
| Ctx | | | | Occipital | | | |
| | | | | Ctx | | | |
| AD 5 Inf | 91.4 | 95.3 | 82.4 | Control | 8.9 | 13.9 | 11.3 |
| Temporal | | | | (Path) 2 | | | |
| Ctx | | | | Occipital | | | |
| | | | | Ctx | | | |
| AD 5 Sup | 25.3 | 35.4 | 33.0 | Control | 2.8 | 4.5 | 3.3 |
| Temporal | | | | (Path) 3 | | | |
| Ctx | | | | Occipital | | | |
| | | | | Ctx | | | |
| AD 6 Inf | 27.4 | 27.7 | 30.4 | Control | 17.2 | 15.9 | 13.9 |
| Temporal | | | | (Path) 4 | | | |
| Ctx | | | | Occipital | | | |
| | | | | Ctx | | | |
| AD 6 Sup | 28.7 | 26.8 | 32.1 | Control 1 | 5.5 | 7.4 | 4.9 |
| Temporal | | | | Parietal Ctx | | | |
| Ctx | | | | | | | |
| Control 1 | 3.4 | 5.9 | 2.8 | Control 2 | 31.9 | 38.7 | 35.6 |
| Temporal | | | | Parietal Ctx | | | |
| Ctx | | | | | | | |
| Control 2 | 34.4 | 42.9 | 28.9 | Control 3 | 14.9 | 20.9 | 15.1 |
| Temporal | | | | Parietal Ctx | | | |
| Ctx | | | | | | | |
| Control 3 | 6.4 | 12.8 | 7.7 | Control | 47.3 | 53.2 | 51.8 |
| Temporal | | | | (Path) 1 | | | |
| Ctx | | | | Parietal Ctx | | | |
| Control 3 | 6.0 | 11.3 | 8.2 | Control | 14.5 | 15.9 | 16.2 |
| Temporal | | | | (Path) 2 | | | |
| Ctx | | | | Parietal Ctx | | | |
| Control | 37.6 | 37.9 | 39.8 | Control | 3.6 | 5.6 | 4.3 |
| (Path) 1 | | | | (Path) 3 | | | |
| Temporal | | | | Parietal Ctx | | | |
| Ctx | | | | | | | |
| Control | 24.1 | 30.1 | 26.8 | Control | 25.2 | 35.4 | 31.6 |
| (Path) 2 | | | | (Path) 4 | | | |
| Temporal | | | | Parietal Ctx | | | |
| Ctx | | | | | | | |

TABLE BOF
Panel 1.3D

| | Rel. Exp. (%) Ag1810, | Rel. Exp. (%) Ag1811, | Rel. Exp. (%) Ag1811, | Rel. Exp. (%) Ag3011, |
|---|---|---|---|---|
| Tissue Name | Run 146338117 | Run 147104851 | Run 165544641 | Run 167927169 |
| Liver adenocarcinoma | 3.8 | 3.3 | 2.3 | 7.0 |
| Pancreas | 0.3 | 0.0 | 0.6 | 0.4 |
| Pancreatic ca. CAPAN 2 | 0.0 | 0.0 | 0.2 | 0.1 |
| Adrenal gland | 1.1 | 2.2 | 1.7 | 1.2 |
| Thyroid | 0.6 | 1.8 | 1.1 | 0.4 |
| Salivary gland | 0.9 | 1.7 | 0.8 | 1.4 |
| Pituitary gland | 1.2 | 1.5 | 0.6 | 0.7 |
| Brain (fetal) | 2.8 | 4.1 | 8.8 | 15.1 |
| Brain (whole) | 48.0 | 49.0 | 100.0 | 93.3 |
| Brain (amygdala) | 32.1 | 47.6 | 84.1 | 48.0 |
| Brain (cerebellum) | 9.9 | 13.9 | 44.4 | 60.7 |
| Brain (hippocampus) | 75.8 | 86.5 | 88.3 | 31.6 |
| Brain (substantia nigra) | 10.8 | 23.0 | 49.7 | 39.8 |
| Brain (thalamus) | 18.0 | 29.9 | 71.7 | 28.5 |
| Cerebral Cortex | 100.0 | 100.0 | 77.4 | 100.0 |
| Spinal cord | 11.3 | 17.8 | 28.5 | 17.8 |
| glio/astro U87-MG | 0.0 | 0.0 | 0.0 | 0.0 |
| glio/astro U-118-MG | 0.0 | 0.0 | 0.0 | 0.0 |
| astrocytoma SW1783 | 0.0 | 0.0 | 0.0 | 0.1 |
| neuro*; met SK-N-AS | 23.7 | 30.4 | 15.4 | 13.3 |
| astrocytoma SF-539 | 0.0 | 0.0 | 0.2 | 0.0 |
| astrocytoma SNB-75 | 0.0 | 0.1 | 0.1 | 0.2 |
| glioma SNB-19 | 0.0 | 0.0 | 0.0 | 0.0 |
| glioma U251 | 0.0 | 0.0 | 0.1 | 0.0 |
| glioma SF-295 | 0.1 | 0.0 | 0.0 | 0.0 |
| Heart (fetal) | 3.9 | 2.5 | 0.9 | 3.0 |
| Heart | 0.6 | 0.6 | 1.5 | 1.4 |
| Skeletal muscle (fetal) | 11.5 | 18.3 | 2.4 | 6.5 |
| Skeletal muscle | 0.2 | 0.1 | 1.0 | 0.4 |
| Bone marrow | 0.9 | 1.8 | 3.0 | 0.5 |
| Thymus | 5.4 | 7.2 | 4.7 | 9.5 |
| Spleen | 14.3 | 25.9 | 17.4 | 11.9 |
| Lymph node | 7.9 | 15.9 | 29.7 | 11.5 |
| Colorectal | 1.5 | 3.0 | 1.1 | 1.6 |
| Stomach | 3.8 | 4.9 | 2.8 | 1.9 |
| Small intestine | 2.6 | 4.6 | 5.3 | 1.6 |
| Colon ca. SW480 | 0.0 | 0.0 | 0.0 | 0.0 |
| Colon ca.* | 0.0 | 0.0 | 0.0 | 0.0 |
| SW620(SW480 met) | | | | |
| Colon ca. HT29 | 0.0 | 0.0 | 0.0 | 0.0 |
| Colon ca. HCT-116 | 0.0 | 0.0 | 0.0 | 0.0 |
| Colon ca. CaCo-2 | 0.0 | 0.1 | 0.0 | 0.0 |
| Colon ca. | 0.7 | 2.0 | 1.1 | 0.7 |
| tissue(ODO3866) | | | | |
| Colon ca. HCC-2998 | 0.0 | 0.0 | 0.2 | 0.0 |
| Gastric ca.* (liver met) | 0.2 | 0.1 | 0.0 | 0.3 |
| NCI-N87 | | | | |
| Bladder | 0.1 | 0.1 | 0.1 | 0.4 |
| Trachea | 1.8 | 3.8 | 3.8 | 0.8 |
| Kidney | 6.3 | 11.8 | 22.2 | 38.2 |
| Kidney (fetal) | 2.0 | 4.5 | 2.8 | 13.5 |
| Renal ca. 786-0 | 0.0 | 0.0 | 0.0 | 0.0 |
| Renal ca. A498 | 0.0 | 0.0 | 0.0 | 0.1 |
| Renal ca. RXF 393 | 0.0 | 0.0 | 0.0 | 0.0 |
| Renal ca. ACHN | 0.0 | 0.0 | 0.0 | 0.0 |
| Renal ca. UO-31 | 0.0 | 0.0 | 0.0 | 0.0 |
| Renal ca. TK-10 | 0.0 | 0.0 | 0.0 | 0.0 |
| Liver | 0.4 | 1.4 | 1.2 | 2.0 |
| Liver (fetal) | 0.4 | 1.3 | 1.0 | 0.6 |
| Liver ca. (hepatoblast) | 0.0 | 0.0 | 0.0 | 0.0 |
| HepG2 | | | | |
| Lung | 6.7 | 11.9 | 9.3 | 8.0 |
| Lung (fetal) | 5.1 | 7.1 | 4.7 | 3.3 |
| Lung ca. (small cell) | 0.0 | 0.0 | 0.0 | 0.0 |
| LX-1 | | | | |
| Lung ca. (small cell) | 4.2 | 3.2 | 1.6 | 3.5 |
| NCI-H69 | | | | |
| Lung ca. (s.cell var.) | 0.0 | 0.1 | 0.0 | 0.0 |
| SHP-77 | | | | |
| Lung ca. (large | 0.0 | 0.0 | 0.0 | 0.0 |
| cell)NCI-H460 | | | | |
| Lung ca. (non-sm. cell) | 0.0 | 0.1 | 0.0 | 0.4 |
| A549 | | | | |
| Lung ca. (non-s.cell) | 0.5 | 0.3 | 0.4 | 0.7 |
| NCI-H23 | | | | |
| Lung ca. (non-s.cell) | 0.0 | 0.0 | 0.0 | 0.1 |
| HOP-62 | | | | |
| Lung ca. (non-s.cl) NCI- | 6.9 | 5.6 | 1.7 | 11.8 |
| H522 | | | | |
| Lung ca. (squam.) SW | 0.0 | 0.0 | 0.0 | 0.0 |
| 900 | | | | |
| Lung ca. (squam.) NCI- | 0.6 | 1.6 | 0.7 | 2.2 |
| H596 | | | | |
| Mammary gland | 3.0 | 4.0 | 1.8 | 2.3 |
| Breast ca.* (pl.ef) MCF-7 | 0.0 | 0.0 | 0.0 | 0.0 |
| Breast ca.* (pl.ef) | 0.0 | 0.0 | 0.0 | 0.0 |
| MDA-MB-231 | | | | |
| Breast ca.* (pl.ef) T47D | 0.0 | 0.0 | 0.0 | 0.0 |
| Breast ca. BT-549 | 0.0 | 0.0 | 0.0 | 0.0 |
| Breast ca. MDA-N | 0.0 | 0.0 | 0.0 | 0.0 |
| Ovary | 2.2 | 1.0 | 0.4 | 0.4 |
| Ovarian ca. OVCAR-3 | 1.2 | 1.1 | 0.8 | 1.5 |
| Ovarian ca. OVCAR-4 | 0.1 | 0.1 | 0.0 | 0.2 |
| Ovarian ca. OVCAR-5 | 0.0 | 0.1 | 0.0 | 0.0 |
| Ovarian ca. OVCAR-8 | 1.4 | 2.3 | 0.9 | 1.1 |
| Ovarian ca. IGROV-1 | 0.0 | 0.0 | 0.1 | 0.1 |
| Ovarian ca.* (ascites) | 0.0 | 0.0 | 0.0 | 0.1 |
| SK-OV-3 | | | | |
| Uterus | 1.8 | 2.7 | 4.0 | 1.4 |
| Placenta | 3.2 | 4.5 | 2.8 | 0.6 |
| Prostate | 0.3 | 0.6 | 0.8 | 0.4 |
| Prostate ca.* (bone | 0.0 | 0.0 | 0.0 | 0.0 |
| met)PC-3 | | | | |
| Testis | 0.4 | 0.3 | 0.4 | 0.1 |
| Melanoma Hs688(A).T | 0.0 | 0.0 | 0.0 | 0.0 |
| Melanoma* (met) | 0.0 | 0.0 | 0.0 | 0.0 |
| Hs688(B).T | | | | |
| Melanoma UACC-62 | 0.0 | 0.0 | 0.0 | 0.0 |
| Melanoma M14 | 0.0 | 0.0 | 0.0 | 0.0 |
| Melanoma LOX IMVI | 0.0 | 0.0 | 0.0 | 0.0 |
| Melanoma* (met) SK- | 0.4 | 0.1 | 0.1 | 0.9 |
| MEL-5 | | | | |
| Adipose | 2.4 | 4.0 | 3.0 | 8.0 |

TABLE BOG
Panel 2D

| | Rel. Exp.(%) | Rel. Exp.(%) | | Rel. Exp.(%) | Rel. Exp.(%) |
|---|---|---|---|---|---|
| Tissue Name | 1604024410 | 147164308 | Tissue Name | 164024410 | 147164308 |
| Normal Colon | 6.8 | 6.3 | Kidney Margin | 36.3 | 37.6 |
| | | | 8120608 | | |
| CC Well to Mod Diff | 1.5 | 1.7 | Kidney Cancer | 3.0 | 2.3 |
| (ODO3866) | | | 8120613 | | |
| CC Margin | 3.0 | 2.9 | Kidney Margin | 100.0 | 100.0 |
| (ODO3866) | | | 8120614 | | |
| CC Gr.2 rectosigmoid | 1.3 | 1.2 | Kidney Cancer | 11.0 | 12.8 |
| (ODO3868) | | | 9010320 | | |
| CC Margin | 0.9 | 1.0 | Kidney Margin | 24.5 | 22.5 |
| (ODO3868) | | | 9010321 | | |
| CC Mod Diff | 3.5 | 2.0 | Normal Uterus | 3.4 | 2.6 |
| (ODO3920) | | | | | |
| CC Margin | 3.9 | 2.1 | Uterus Cancer | 4.8 | 3.7 |
| (ODO3920) | | | 064011 | | |
| CC Gr.2 ascend colon | 8.9 | 6.9 | Normal Thyroid | 3.0 | 1.3 |
| (ODO3921) | | | | | |
| CC Margin | 3.7 | 2.3 | Thyroid Cancer | 2.4 | 2.0 |
| (ODO3921) | | | 064010 | | |
| CC from Partial | 5.3 | 3.7 | Thyroid Cancer | 4.5 | 1.8 |
| Hepatectomy | | | A302152 | | |
| (ODO4309) Mets | | | | | |
| Liver Margin | 3.0 | 3.0 | Thyroid Margin | 9.5 | 8.0 |
| (ODO4309) | | | A302153 | | |
| Colon mets to lung | 9.0 | 3.2 | Normal Breast | 14.1 | 12.1 |
| (OD04451-01) | | | | | |
| Lung Margin | 6.2 | 4.5 | Breast Cancer | 0.7 | 0.5 |
| (OD04451-02) | | | (OD04566) | | |
| Normal Prostate 6546- | 5.6 | 1.1 | Breast Cancer | 2.5 | 3.0 |
| 1 | | | (OD04590-01) | | |
| Prostate Cancer | 3.0 | 3.2 | Breast Cancer Mets | 16.6 | 19.6 |
| (OD04410) | | | (OD04590-03) | | |
| Prostate Margin | 4.8 | 2.9 | Breast Cancer | 9.4 | 7.3 |
| (OD04410) | | | Metastasis | | |
| | | | (OD04655-05) | | |
| Prostate Cancer | 4.4 | 2.5 | Breast Cancer | 9.5 | 7.1 |
| (OD04720-01) | | | 064006 | | |
| Prostate Margin | 11.4 | 7.5 | Breast Cancer 1024 | 6.9 | 5.6 |
| (OD04720-02) | | | | | |
| Normal Lung 061010 | 19.3 | 17.8 | Breast Cancer | 3.4 | 1.7 |
| | | | 9100266 | | |
| Lung Met to Muscle | 3.3 | 2.7 | Breast Margin | 2.8 | 2.5 |
| (ODO4286) | | | 9100265 | | |
| Muscle Margin | 2.1 | 0.70 | Breast Cancer | 8.3 | 5.9 |
| (ODO4286) | | | A209073 | | |
| Lung Malignant | 4.7 | 5.6 | Breast Margin | 5.6 | 3.7 |
| Cancer (OD03126) | | | A209073 | | |
| Lung Margin | 17.9 | 16.4 | Normal Liver | 1.5 | 0.7 |
| (OD03126) | | | | | |
| Lung Cancer | 8.2 | 8.1 | Liver Cancer | 1.8 | 2.2 |
| (OD04404) | | | 064003 | | |
| Lung Margin | 13.8 | 9.8 | Liver Cancer 1025 | 2.2 | 1.5 |
| (OD04404) | | | | | |
| Lung Cancer | 1.9 | 1.4 | Liver Cancer 1026 | 2.0 | 2.1 |
| (OD04565) | | | | | |
| Lung Margin | 8.0 | 4.2 | Liver Cancer 6004- | 3.2 | 2.0 |
| (OD04565) | | | T | | |
| Lung Cancer | 8.1 | 7.5 | Liver Tissue 6004- | 1.4 | 1.8 |
| (OD04237-01) | | | N | | |
| Lung Margin | 12.7 | 11.1 | Liver Cancer 6005- | 3.6 | 2.3 |
| (OD04237-02) | | | T | | |
| Ocular Mel Met to | 0.6 | 0.9 | Liver Tissue 6005- | 2.9 | 1.5 |
| Liver (ODO4310) | | | N | | |
| Liver Margin | 2.4 | 2.1 | Normal Bladder | 3.1 | 2.6 |
| (ODO4310) | | | | | |
| Melanoma Mets to | 4.5 | 3.8 | Bladder Cancer | 3.3 | 1.4 |
| Lung (OD04321) | | | 1023 | | |
| Lung Margin | 15.2 | 18.7 | Bladder Cancer | 2.7 | 2.6 |
| (OD04321) | | | A302173 | | |
| Normal Kidney | 54.0 | 45.1 | Bladder Cancer | 4.8 | 3.1 |
| | | | (OD04718-01) | | |
| Kidney Ca, Nuclear | 2.2 | 1.6 | Bladder Normal | 5.7 | 3.8 |
| grade 2 (OD04338) | | | Adjacent | | |
| | | | (OD04718-03) | | |
| Kidney Margin | 23.3 | 14.4 | Normal Ovary | 1.2 | 1.7 |
| (OD04338) | | | | | |
| Kidney Ca Nuclear | 2.7 | 1.0 | Ovarian Cancer | 7.2 | 5.0 |
| grade 1/2 (OD04339) | | | 064008 | | |
| Kidney Margin | 94.0 | 84.1 | Ovarian Cancer | 1.5 | 0.7 |
| (OD04339) | | | (OD04768-07) | | |
| Kidney Ca, Clear cell | 27.0 | 22.8 | Ovary Margin | 3.4 | 3.8 |
| type (OD04340) | | | (OD04768-08) | | |
| Kidney Margin | 54.7 | 52.5 | Normal Stomach | 8.0 | 7.1 |
| (OD04340) | | | | | |
| Kidney Ca, Nuclear | 2.7 | 1.7 | Gastric Cancer | 5.1 | 3.6 |
| grade 3 (OD04348) | | | 9060358 | | |
| Kidney Margin | 15.0 | 9.2 | Stomach Margin | 8.7 | 7.9 |
| (OD04348) | | | 9060359 | | |
| Kidney Cancer | 11.5 | 5.6 | Gastric Cancer | 7.1 | 6.6 |
| (OD04622-01) | | | 9060395 | | |
| Kidney Margin | 17.6 | 9.0 | Stomach Margin | 14.5 | 13.3 |
| (OD04622-03) | | | 9060394 | | |
| Kidney Cancer | 0.4 | 0.5 | Gastric Cancer | 3.3 | 2.9 |
| (OD04450-01) | | | 9060397 | | |
| Kidney Margin | 18.7 | 12.1 | Stomach Margin | 7.2 | 5.0 |
| (OD04450-03) | | | 9060396 | | |
| Kidney Cancer | 1.7 | 1.0 | Gastric Cancer | 21.8 | 10.4 |
| 8120607 | | | 064005 | | |

TABLE BOH
Panel 4D

| | Rel. | Rel. | Rel. | | Rel. | Rel. | Rel. |
|---|---|---|---|---|---|---|---|
| | Exp.(%) | Exp.(%) | Exp.(%) | | Exp.(%) | Exp.(%) | Exp.(%) |
| | Ag1810, | Ag1811, | Ag3011, | | Ag1810, | Ag1811, | Ag3011, |
| | Run | Run | Run | | Run | Run | Run |
| Tissue Name | 162733751 | 147164258 | 164043491 | Tissue Name | 162733751 | 147164258 | 164043491 |
| Secondary Th1 act | 42.3 | 40.9 | 30.1 | HUVEC IL-1beta | 3.8 | 4.5 | 4.5 |
| Secondary Th2 act | 37.1 | 43.8 | 32.5 | HUVEC IFN | 18.8 | 15.7 | 13.6 |
| | | | | gamma | | | |
| Secondary Tr1 act | 47.6 | 54.0 | 35.8 | HUVEC TNF | 1.4 | 1.1 | 0.6 |
| | | | | alpha + IFN | | | |
| | | | | gamma | | | |
| Secondary Th1 rest | 8.0 | 11.5 | 7.2 | HUVEC TNF | 2.5 | 1.8 | 1.2 |
| | | | | alpha + IL4 | | | |
| Secondary Th2 rest | 13.4 | 11.6 | 11.9 | HUVEC IL-11 | 12.2 | 12.0 | 7.5 |
| Secondary Tr1 rest | 12.8 | 12.8 | 9.3 | Lung | 13.1 | 11.3 | 10.2 |
| | | | | Microvascular EC | | | |
| | | | | none | | | |
| Primary Th1 act | 21.3 | 18.9 | 14.9 | Lung | 3.8 | 4.4 | 2.5 |
| | | | | Microvascular EC | | | |
| | | | | TNFalpha + IL- | | | |
| | | | | 1beta | | | |
| Primary Th2 act | 29.5 | 39.8 | 26.1 | Microvascular | 37.1 | 27.4 | 29.3 |
| | | | | Dermal EC none | | | |
| Primary Tr1 act | 33.9 | 42.3 | 27.7 | Microvascular | 10.7 | 9.5 | 7.4 |
| | | | | Dermal EC | | | |
| | | | | TNFalpha + IL- | | | |
| | | | | 1beta | | | |
| Primary Th1 rest | 44.8 | 36.1 | 34.6 | Bronchial | 0.0 | 0.0 | 0.0 |
| | | | | epithelium | | | |
| | | | | TNFalpha + | | | |
| | | | | IL1beta | | | |
| Primary Th2 rest | 25.9 | 20.6 | 28.9 | Small airway | 0.0 | 0.0 | 0.0 |
| | | | | epithelium none | | | |
| Primary Tr1 rest | 22.2 | 11.0 | 15.1 | Small airway | 0.0 | 0.0 | 0.0 |
| | | | | epithelium | | | |
| | | | | TNFalpha + IL- | | | |
| | | | | 1beta | | | |
| CD45RA CD4 | 11.6 | 11.3 | 8.7 | Coronery artery | 0.3 | 0.3 | 0.3 |
| lymphocyte act | | | | SMC rest | | | |
| CD45RO CD4 | 41.8 | 35.1 | 29.3 | Coronery artery | 0.2 | 0.1 | 0.1 |
| lymphocyte act | | | | SMC TNFalpha + | | | |
| | | | | IL-1beta | | | |
| CD8 lymphocyte | 31.4 | 24.1 | 23.3 | Astrocytes rest | 0.0 | 0.0 | 0.0 |
| act | | | | | | | |
| Secondary CD8 | 42.6 | 25.3 | 31.6 | Astrocytes | 0.0 | 0.0 | 0.0 |
| lymphocyte rest | | | | TNFalpha + IL- | | | |
| | | | | 1beta | | | |
| Secondary CD8 | 28.7 | 17.1 | 20.4 | KU-812 | 0.1 | 0.3 | 0.1 |
| lymphocyte act | | | | (Basophil) rest | | | |
| CD4 lymphocyte | 6.2 | 3.6 | 4.6 | KU-812 | 5.6 | 2.9 | 2.9 |
| none | | | | (Basophil) | | | |
| | | | | PMA/inonomycin | | | |
| 2ry | 20.4 | 16.0 | 18.9 | CCD1106 | 0.0 | 0.1 | 0.0 |
| Th1/Th2/Tr1_anti- | | | | (Keratinocytes) | | | |
| CD95 CH11 | | | | none | | | |
| LAK cells rest | 12.9 | 9.6 | 7.9 | CCD1106 | 0.0 | 0.0 | 0.0 |
| | | | | (Keratinocytes) | | | |
| | | | | TNFalpha + IL- | | | |
| | | | | 1beta | | | |
| LAK cells IL-2 | 38.7 | 28.3 | 30.1 | Liver cirrhosis | 0.6 | 0.5 | 0.8 |
| LAK cells IL-2 + IL- | 27.4 | 18.4 | 20.2 | Lupus kidney | 0.3 | 0.5 | 0.4 |
| 12 | | | | | | | |
| LAK cells IL- | 42.9 | 29.3 | 26.6 | NCI-H292 none | 0.0 | 0.0 | 0.0 |
| 2 + IFN gamma | | | | | | | |
| LAK cells IL-2 + | 45.7 | 22.7 | 20.3 | NCI-H292 IL-4 | 0.0 | 0.0 | 0.0 |
| IL-18 | | | | | | | |
| LAK cells | 31.6 | 26.6 | 20.6 | NCI-H292 IL-9 | 0.0 | 0.0 | 0.0 |
| PMA/ionomycin | | | | | | | |
| NK Cells IL-2 rest | 21.9 | 17.9 | 15.5 | NCI-H292 IL-13 | 0.1 | 0.0 | 0.0 |
| Two Way MLR 3 | 20.3 | 19.9 | 14.4 | NCI-H292 IFN | 0.0 | 0.0 | 0.0 |
| day | | | | gamma | | | |
| Two Way MLR 5 | 23.2 | 16.2 | 15.1 | HPAEC none | 13.0 | 14.5 | 9.4 |
| day | | | | | | | |
| Two Way MLR 7 | 16.7 | 16.3 | 17.9 | HPAEC TNF | 4.6 | 5.6 | 4.4 |
| day | | | | alpha + IL-1 beta | | | |
| PBMC rest | 5.9 | 4.1 | 4.5 | Lung fibroblast | 0.0 | 0.0 | 0.0 |
| | | | | none | | | |
| PBMC PWM | 79.6 | 54.7 | 84.1 | Lung fibroblast | 0.0 | 0.0 | 0.0 |
| | | | | TNF alpha + IL-1 | | | |
| | | | | beta | | | |
| PBMC PHA-L | 39.8 | 31.6 | 39.0 | Lung fibroblast IL- | 0.0 | 0.0 | 0.0 |
| | | | | 4 | | | |
| Ramos (B cell) | 10.2 | 7.5 | 8.7 | Lung fibroblast IL- | 0.0 | 0.0 | 0.0 |
| none | | | | 9 | | | |
| Ramos (B cell) | 20.9 | 12.2 | 13.6 | Lung fibroblast IL- | 0.0 | 0.0 | 0.0 |
| ionomycin | | | | 13 | | | |
| B lymphocytes | 96.6 | 100.0 | 100.0 | Lung fibroblast | 0.1 | 0.0 | 0.0 |
| PWM | | | | IFN gamma | | | |
| B lymphocytes | 100.0 | 59.9 | 53.2 | Dermal fibroblast | 0.0 | 0.0 | 0.0 |
| CD40L and IL-4 | | | | CCD1070 rest | | | |
| EOL-1 dbcAMP | 6.2 | 5.4 | 3.9 | Dermal fibroblast | 27.0 | 22.1 | 22.5 |
| | | | | CCD1070 TNF | | | |
| | | | | alpha | | | |
| EOL-1 dbcAMP | 61.1 | 46.3 | 38.4 | Dermal fibroblast | 0.0 | 0.1 | 0.0 |
| PMA/ionomycin | | | | CCD1070 IL-1 | | | |
| | | | | beta | | | |
| Dendritic cells none | 3.1 | 2.2 | 1.7 | Dermal fibroblast | 0.0 | 0.0 | 0.0 |
| | | | | IFN gamma | | | |
| Dendritic cells LPS | 5.3 | 3.8 | 4.0 | Dermal fibroblast | 0.0 | 0.0 | 0.0 |
| | | | | IL-4 | | | |
| Dendritic cells anti- | 1.1 | 0.9 | 1.3 | IBD Colitis 2 | 0.7 | 0.9 | 0.6 |
| CD40 | | | | | | | |
| Monocytes rest | 1.3 | 0.7 | 0.9 | IBD Crohn's | 0.4 | 0.2 | 0.2 |
| Monocytes LPS | 1.4 | 1.0 | 1.1 | Colon | 8.3 | 6.8 | 5.7 |
| Macrophages rest | 1.8 | 1.4 | 1.7 | Lung | 8.5 | 7.5 | 5.8 |
| Macrophages LPS | 0.7 | 0.7 | 0.4 | Thymus | 21.2 | 27.2 | 21.3 |
| HUVEC none | 11.4 | 7.8 | 7.5 | Kidney | 9.3 | 8.3 | 7.6 |
| HUVEC starved | 17.7 | 13.7 | 10.0 | | | | |

TABLE BOI
Panel CNS_1

| | Rel. Exp. (%) Ag1811, Run | | Rel. Exp. (%) Ag1811, Run |
|---|---|---|---|
| Tissue Name | 171628390 | Tissue Name | 171628390 |
| BA4 Control | 27.7 | BA17 PSP | 31.9 |
| BA4 Control2 | 52.1 | BA17 PSP2 | 10.1 |
| BA4 Alzheimer's2 | 6.0 | Sub Nigra Control | 36.1 |
| BA4 Parkinson's | 51.4 | Sub Nigra Control2 | 20.3 |
| BA4 Parkinson's2 | 93.3 | Sub Nigra Alzheimer's2 | 5.9 |
| BA4 Huntington's | 13.2 | Sub Nigra Parkinson's2 | 32.5 |
| BA4 Huntington's2 | 7.7 | Sub Nigra Huntington's | 35.1 |
| BA4 PSP | 7.7 | Sub Nigra Huntington's2 | 18.6 |
| BA4 PSP2 | 17.3 | Sub Nigra PSP2 | 5.5 |
| BA4 Depression | 19.2 | Sub Nigra Depression | 8.8 |
| BA4 Depression2 | 11.1 | Sub Nigra Depression2 | 3.0 |
| BA7 Control | 42.6 | Glob Palladus Control | 17.4 |
| BA7 Control2 | 35.6 | Glob Palladus Control2 | 19.3 |
| BA7 Alzheimer's2 | 5.9 | Glob Palladus Alzheimer's | 4.0 |
| BA7 Parkinson's | 21.2 | Glob Palladus | 8.5 |
| | | Alzheimer's2 | |
| BA7 Parkinson's2 | 61.6 | Glob Palladus Parkinson's | 100.0 |
| BA7 Huntington's | 61.1 | Glob Palladus | 15.3 |
| | | Parkinson's2 | |
| BA7 Huntington's2 | 75.3 | Glob Palladus PSP | 3.9 |
| BA7 PSP | 29.3 | Glob Palladus PSP2 | 8.4 |
| BA7 PSP2 | 17.6 | Glob Palladus Depression | 4.1 |
| BA7 Depression | 13.6 | Temp Pole Control | 14.0 |
| BA9 Control | 21.2 | Temp Pole Control2 | 70.7 |
| BA9 Control2 | 90.1 | Temp Pole Alzheimer's | 3.8 |
| BA9 Alzheimer's | 4.0 | Temp Pole Alzheimer's2 | 5.4 |
| BA9 Alzheimer's2 | 12.6 | Temp Pole Parkinson's | 32.8 |
| BA9 Parkinson's | 40.9 | Temp Pole Parkinson's2 | 28.1 |
| BA9 Parkinson's2 | 61.1 | Temp Pole Huntington's | 38.4 |
| BA9 Huntington's | 67.4 | Temp Pole PSP | 3.8 |
| BA9 Huntington's2 | 17.8 | Temp Pole PSP2 | 2.7 |
| BA9 PSP | 13.8 | Temp Pole Depression2 | 8.9 |
| BA9 PSP2 | 5.1 | Cing Gyr Control | 60.7 |
| BA9 Depression | 14.0 | Cing Gyr Control2 | 29.7 |
| BA9 Depression2 | 14.5 | Cing Gyr Alzheimer's | 16.7 |
| BA17 Control | 61.6 | Cing Gyr Alzheimer's2 | 5.9 |
| BA17 Control2 | 56.3 | Cing Gyr Parkinson's | 43.5 |
| BA17 Alzheimer's2 | 9.0 | Cing Gyr Parkinson's2 | 45.7 |
| BA17 Parkinson's | 49.3 | Cing Gyr Huntington's | 78.5 |
| BA17 Parkinson's2 | 74.7 | Cing Gyr Huntington's2 | 28.5 |
| BA17 Huntington's | 44.4 | Cing Gyr PSP | 13.1 |
| BA17 | 42.9 | Cing Gyr PSP2 | 6.1 |
| Huntington's2 | | | |
| BA17 Depression | 14.5 | Cing Gyr Depression | 7.7 |
| BA17 Depression2 | 37.9 | Cing Gyr Depression2 | 14.1 |

AI_comprehensive panel_v1.0 Summary: Ag3011 CG56761-01 gene expression is upregulated in arthritis tissue as compared to normal joint tissue. It is also expressed in matched control gut tissue and at low levels in pulmonary tissue. The consistent induction of this transcript in arthritic tissue suggests that the protein encoded for by this transcript may be involved in the pathological processes associated with arthritis and may serve as a relevant target for therapeutic intervention.

CNS_neurodegeneration_v1.0 Summary: Ag1810/Ag1811/Ag3011 Results from three experiments using different probe/primer sets are in excellent agreement. This panel confirms the expression of the CG56761-01 gene at low levels in the brains of an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel_{—}1.3D Summary: Ag1810/Ag1811/Ag3011 Three experiments with three different probe and primer sets produce results that are in excellent agreement. The CG56761-01 gene is expressed at moderate to high levels in all regions of the central nervous system examined, including amygdala, hippocampus, substantia nigra, thalamus, cerebellum, cerebral cortex, and spinal cord. Thus, expression of this gene may be used to distinguish brain from the other samples on this panel. In addition, this gene may play a role in central nervous system disorders Such as Alzheimer's disease, Parkinson's disease, epilepsy, multiple sclerosis, schizophrenia and depression. The CG56761-01 gene encodes a protein that contains ankyrin repeats.

Interestingly, expression of this gene appears to be highly down-regulated in CNS cancer cell lines. Thus, modulation of the activity of this gene product may be of benefit in the treatment of cancers of the central nervous system.

Panel 2D Summary: Ag1810/Ag1811 Results from two experiments using different probe/primer sets are in excellent agreement. Expression of the CG56761-01 gene is highest in a sample of normal kidney tissue (CT=27). Expression of this gene is upregulated in matched normal kidney, lung, thyroid, ovary and stomach tissues when compared to the adjacent tumor samples. Thus, expression of this gene may be used to distinguish normal kidney, lung, thyroid, ovary and stomach from cancers of these tissues. In addition, therapeutic modulation of the activity of this gene or its protein product, using small molecule drugs, antibodies or protein therapeutics, may be of use in the treatment of these cancers.

Panel 4D Summary: Ag1810/Ag1811/Ag3011 Three experiments with two different probe and primer sets produce results that are in excellent agreement. High expression of CG56761-01 is detected in samples derived from PWM and CD40L/IL4 treated B lymphocytes (CTs=26), PWM (CT=26.4) and PHL-A (CT=27.4) treated PMBC. Thus, expression of this gene could be used to differentiate between these samples and other samples on this panel. Expression of this transcript in B cells suggests that this gene may be involved in rheumatic disease including rheumatoid arthritis, lupus, osteoarthritis, and hyperproliferative B cell disorders.

Panel CNS_i Summary: Ag1811 This panel confirms the expression of CG56761-01 gene at low levels in the brains of an independent group of individuals. Please see Panel 1.3D for a discussion of the potential utility of this gene in treatment of central nervous system disorders.

NOV73

Expression of NOV73/CG57313-01 was assessed using the primer-probe set Ag3185, described in Table BAA.

TABLE BAA
Probe Name Ag3185

| Primers | Sequences | Length | Start Position | SEQ ID NO |
|---|---|---|---|---|
| Forward | 5′-ccatcttctgtgggacagttac-3′ | 22 | 779 | 485 |
| Probe | TET-5′-catgtatatacagccaggaaacagtcca-3′-TAMRA | 28 | 804 | 486 |
| Reverse | 5′-agaagtttgccctcattctgat-3′ | 22 | 833 | 487 |

CNS_neurodegeneration_v1.0 Summary: Ag3185 Expression of this gene is low/undetectable (CTs>35) across all of the samples on this panel (data not shown).

Panel 1.3D Summary: Ag3185 Expression of this gene is low/undetectable (CTs>35) across all of the samples on this panel (data not shown).

Panel 4D Summary: Ag31185 Expression of this gene is low/undetectable (CTs>35) across all of the samples on this panel (data not shown).

NOV74

Expression of NOV74/CG57315-01 was assessed using the primer-probe set Ag3186, described in Table BBA. Results of the RTQ-PCR runs are shown in Tables BBB, and BBC.

TABLE BBA
Probe Name Ag3186

| Primers | Sequences | Length | Start Position | SEQ ID NO |
|---|---|---|---|---|
| Forward | 5′-gagttgcattgtgttgttgttg-3′ | 22 | 610 | 488 |
| Probe | TET-5′-tcattttccttgcatcacttcttctca-3′-TAMRA | 27 | 638 | 489 |
| Reverse | 5′-caatgaagccatatgacacaag-3′ | 22 | 667 | 490 |

TABLE BBB
CNS_neurodegeneration_v1.0

| | Rel. Exp. (%) Ag3186, Run | | Rel. Exp. (%) Ag3186, Run |
|---|---|---|---|
| Tissue Name | 209859516 | Tissue Name | 209859516 |
| AD 1 Hippo | 32.5 | Control (Path) 3 Temporal | 0.0 |
| | | Ctx | |
| AD 2 Hippo | 100.0 | Control (Path) 4 Temporal | 0.0 |
| | | Ctx | |
| AD 3 Hippo | 29.9 | AD 1 Occipital Ctx | 0.0 |
| AD 4 Hippo | 38.7 | AD 2 Occipital Ctx | 0.0 |
| | | (Missing) | |
| AD 5 hippo | 0.0 | AD 3 Occipital Ctx | 0.0 |
| AD 6 Hippo | 76.8 | AD 4 Occipital Ctx | 5.4 |
| Control 2 Hippo | 55.5 | AD 5 Occipital Ctx | 0.0 |
| Control 4 Hippo | 92.7 | AD 6 Occipital Ctx | 0.0 |
| Control (Path) 3 Hippo | 32.8 | Control 1 Occipital Ctx | 0.0 |
| AD 1 Temporal Ctx | 0.0 | Control 2 Occipital Ctx | 0.0 |
| AD 2 Temporal Ctx | 13.2 | Control 3 Occipital Ctx | 7.6 |
| AD 3 Temporal Ctx | 0.0 | Control 4 Occipital Ctx | 0.0 |
| AD 4 Temporal Ctx | 0.0 | Control (Path) 1 Occipital | 42.0 |
| | | Ctx | |
| AD 5 Inf Temporal Ctx | 0.0 | Control (Path) 2 Occipital | 0.0 |
| | | Ctx | |
| AD 5 SupTemporal Ctx | 87.1 | Control (Path) 3 Occipital | 0.0 |
| | | Ctx | |
| AD 6 Inf Temporal Ctx | 0.0 | Control (Path) 4 Occipital | 0.0 |
| | | Ctx | |
| AD 6 Sup Temporal Ctx | 0.0 | Control 1 Parietal Ctx | 0.0 |
| Control 1 Temporal Ctx | 0.0 | Control 2 Parietal Ctx | 0.0 |
| Control 2 Temporal Ctx | 0.0 | Control 3 Parietal Ctx | 0.0 |
| Control 3 Temporal Ctx | 11.2 | Control (Path) 1 Parietal | 7.0 |
| | | Ctx | |
| Control 4 Temporal Ctx | 0.0 | Control (Path) 2 Parietal | 0.0 |
| | | Ctx | |
| Control (Path) 1 Temporal | 16.2 | Control (Path) 3 Parietal | 0.0 |
| Ctx | | Ctx | |
| Control (Path) 2 Temporal | 0.0 | Control (Path) 4 Parietal | 0.0 |
| Ctx | | Ctx | |

TABLE BBC
Panel 1.3D

| | Rel. Exp. (%) Ag3186, Run | | Rel. Exp. (%) Ag3186, Run |
|---|---|---|---|
| Tissue Name | 167994623 | Tissue Name | 167994623 |
| Liver adenocarcinoma | 20.4 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 11.0 | Liver | 0.0 |
| Brain (whole) | 0.0 | Liver (fetal) | 0.0 |
| Brain (amygdala) | 3.6 | Liver ca. (hepatoblast) | 0.0 |
| | | HepG2 | |
| Brain (cerebellum) | 0.0 | Lung | 0.0 |
| Brain (hippocampus) | 15.4 | Lung (fetal) | 0.0 |
| Brain (substantia nigra) | 0.0 | Lung ca. (small cell) LX-1 | 0.0 |
| Brain (thalamus) | 14.7 | Lung ca. (small cell) NCI- | 0.0 |
| | | H69 | |
| Cerebral Cortex | 0.0 | Lung ca. (s.cell var.) SHP- | 0.0 |
| | | 77 | |
| Spinal cord | 38.2 | Lung ca. (large cell)NCI- | 0.0 |
| | | H460 | |
| glio/astro U87-MG | 0.0 | Lung ca. (non-sm. cell) | 9.7 |
| | | A549 | |
| glio/astro U-118-MG | 0.0 | Lung ca. (non-s.cell) NCI- | 36.3 |
| | | H23 | |
| astrocytoma SW1783 | 0.0 | Lung ca. (non-s.cell) HOP- | 9.7 |
| | | 62 | |
| neuro*; met SK-N-AS | 0.0 | Lung ca. (non-s.cl) NCI- | 0.0 |
| | | H522 | |
| astrocytoma SF-539 | 15.6 | Lung ca. (squam.) SW 900 | 0.0 |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl.ef) MCF-7 | 21.6 |
| glioma SF-295 | 0.0 | Breast ca.* (pl.ef) MDA- | 0.0 |
| | | MB-231 | |
| Heart (fetal) | 0.0 | Breast ca.* (pl.ef) T47D | 0.0 |
| Heart | 0.0 | Breast ca. BT-549 | 9.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 8.0 |
| Lymph node | 0.0 | Ovarian ca. OVCAR-8 | 0.0 |
| Colorectal | 24.7 | Ovarian ca. IGROV-1 | 0.0 |
| Stomach | 0.0 | Ovarian ca.* (ascites) SK- | 100.0 |
| | | OV-3 | |
| Small intestine | 0.0 | Uterus | 0.0 |
| Colon ca. SW480 | 16.2 | Placenta | 0.0 |
| Colon ca.* SW620 (SW480 | 0.0 | Prostate | 0.0 |
| met) | | | |
| Colon ca. HT29 | 0.0 | Prostate ca.* (bone met)PC-3 | 0.0 |
| Colon ca. HCT-116 | 0.0 | Testis | 32.1 |
| Colon ca. CaCo-2 | 0.0 | Melanoma Hs688(A).T | 0.0 |
| Colon ca. tissue(ODO3866) | 0.0 | Melanoma* (met) | 0.0 |
| | | Hs688(B).T | |
| Colon ca. HCC-2998 | 0.0 | Melanoma UACC-62 | 0.0 |
| Gastric ca.* (liver met) NCI- | 0.0 | Melanoma M14 | 0.0 |
| N87 | | | |
| Bladder | 21.6 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

CNS_neurodegeneration_v1.0 Summary: Ag3186 This panel demonstrates the expression of the CG57315-01 gene at low levels in the brains of a group of several individuals. However, no differential expression of this gene was detected between Alzheimer's diseased postmortem brains and those of non-demented controls in this experiment. The CG57315-01 gene encodes a putative GPCR. Several neurotransmitter receptors are GPCRs, including the dopamine receptor family, the serotonin receptor family, the GABAB receptor, muscarinic acetylcholine receptors, and others; thus this GPCR may represent a novel neurotransmitter receptor. Targeting various neurotransmitter receptors (dopamine, serotonin) has proven to be an effective therapy in psychiatric illnesses such as schizophrenia, bipolar disorder, and depression. Furthermore, the cerebral cortex and hippocampus are regions of the brain that are known to be involved in Alzheimer's disease, seizure disorders, and in the normal process of memory formation. Therefore, therapeutic modulation of this gene or its protein product may be beneficial in the treatment of one or more of these diseases, as may stimulation and/or blockade of the receptor coded for by the gene.

Panel 1.3D Summary: Ag3186 Low levels of expression of the CG57315-01 gene are seen exclusively in an ovarian cancer sample (CT=34.7). Therefore, expression of this gene may be used to distinguish ovarian cancer cell lines from the other samples on this panel. Furthermore, therapeutic modulation of the activity of the GPCR encoded by this gene may be beneficial in the treatment of ovarian cancer.

Panel 4D Summary: Ag3186 Expression of CG57315-01 is low/undetectable (CTs>35) across all of the samples on this panel (data not shown).

NOV75

Expression of NOV75/CG57317-01 was assessed using the primer-probe set Ag3187, described in Table BCA. Results of the RTQ-PCR runs are shown in Tables BCB.

TABLE BCA
Probe Name Ag3187

| Primers | Sequences | Length | Start Position | SEQ ID NO |
|---|---|---|---|---|
| Forward | 5′-tttgtggctgttttgatatcct-3′ | 22 | 172 | 491 |
| Probe | TET-5′-tctccttacccttgtgggaaatacag-3′-TAMRA | 26 | 195 | 492 |
| Reverse | 5′-gggtctacagagcagatcagaa-3′ | 22 | 227 | 493 |

TABLE BCB
Panel 1.3D

| | Rel. Exp. (%) Ag3187, Run | | Rel. Exp. (%) Ag3187, Run |
|---|---|---|---|
| Tissue Name | 167994630 | Tissue Name | 167994630 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 8.1 | Lung (fetal) | 0.0 |
| Brain (substantia nigra) | 0.0 | Lung ca. (small cell) LX-1 | 0.0 |
| Brain (thalamus) | 0.0 | Lung ca. (small cell) NCI- | 0.0 |
| | | H69 | |
| Cerebral Cortex | 0.0 | Lung ca. (s.cell var.) SHP- | 0.0 |
| | | 77 | |
| Spinal cord | 0.0 | Lung ca. (large cell)NCI- | 0.0 |
| | | H460 | |
| glio/astro U87-MG | 0.0 | Lung ca. (non-sm. cell) | 0.0 |
| | | A549 | |
| glio/astro U-118-MG | 0.0 | Lung ca. (non-s.cell) NCI- | 27.5 |
| | | H23 | |
| astrocytoma SW1783 | 5.9 | Lung ca. (non-s.cell) HOP- | 0.0 |
| | | 62 | |
| neuro*; met SK-N-AS | 0.0 | Lung ca. (non-s.cl) NCI- | 5.9 |
| | | H522 | |
| astrocytoma SF-539 | 0.0 | Lung ca. (squam.) SW 900 | 0.0 |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl.ef) MCF-7 | 0.0 |
| glioma SF-295 | 0.0 | Breast ca.* (pl.ef) MDA- | 0.0 |
| | | MB-231 | |
| Heart (fetal) | 0.0 | Breast ca.* (pl.ef) T47D | 0.0 |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 9.3 | Ovarian ca. OVCAR-5 | 0.0 |
| Lymph node | 0.0 | Ovarian ca. OVCAR-8 | 0.0 |
| Colorectal | 16.0 | Ovarian ca. IGROV-1 | 0.0 |
| Stomach | 0.0 | Ovarian ca.* (ascites) SK- | 100.0 |
| | | OV-3 | |
| Small intestine | 0.0 | Uterus | 0.0 |
| Colon ca. SW480 | 0.0 | Placenta | 0.0 |
| Colon ca.* SW620(SW480 | 0.0 | Prostate | 0.0 |
| met) | | | |
| Colon ca. HT29 | 0.0 | Prostate ca.* (bone met)PC-3 | 0.0 |
| Colon ca. HCT-116 | 0.0 | Testis | 0.0 |
| Colon ca. CaCo-2 | 0.0 | Melanoma Hs688(A).T | 0.0 |
| Colon ca. tissue(ODO3866) | 0.0 | Melanoma* (met) | 0.0 |
| | | Hs688(B).T | |
| Colon ca. HCC-2998 | 0.0 | Melanoma UACC-62 | 0.0 |
| Gastric ca.* (liver met) NCI- | 0.0 | Melanoma M14 | 0.0 |
| N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

Panel 1.3D Summary: Ag3187 Significant expression of CG57317-01 gene is seen exclusively in an ovarian cancer sample (CT=34.12). Therefore, expression of this gene may be used to distinguish ovarian cancers from the other samples on this panel. Furthermore, therapeutic modulation of the activity of the GPCR encoded by this gene may be beneficial in the treatment of ovarian cancer.

Panel 4D Summary: Ag3187 Expression of CG57317-01 gene is low/undetectable (CTs>35) across all of the samples on this panel (data not shown).

NOV77

Expression of NOV77/CG57419-01 was assessed using the primer-probe set Ag3233, described in Table BDA.

TABLE BDA
Probe Name Ag3233

| Primers | Sequences | Length | Start Position | SEQ ID NO |
|---|---|---|---|---|
| Forward | 5′-tggccatgtatttggtgactat-3′ | 22 | 128 | 494 |
| Probe | TET-5′-aggaaacaccctcattcttcttctga-3′-TAMRA | 26 | 153 | 495 |
| Reverse | 5′-tatgaagcctgttgtccagtct-3′ | 22 | 181 | 496 |

CNS_neurodegeneration_v1.0 Summary: Ag3233 Expression of this gene is low/undetectable (CTs>35) across all of the samples on this panel (data not shown).

General_screening_panel_v1.4 Summary: Ag3233 Expression of this gene is low/undetectable (CTs>35) across all of the samples on this panel (data not shown).

Panel 2.2 Summary: Ag3233 Expression of this gene is low/undetectable (CTs>35) across all of the samples on this panel (data not shown).

NOV78

Expression of NOV78/CG57425-01 was assessed using the primer-probe sets Ag1703 and Ag1747, described in Tables BEA and BEB. Results of the RTQ-PCR runs are shown in Table BEC.

TABLE BEA
Probe Name Ag1703

| Primers | Sequences | Length | Start Position | SEQ ID NO |
|---|---|---|---|---|
| Forward | 5′-gaggtgctttcaacaaaggaat-3′ | 22 | 108 | 497 |
| Probe | TET-5′-tcggttactcccttactgaatcccttca-3′-TAMRA | 28 | 64 | 498 |
| Reverse | 5′-gcttgtttcacttgctgatttc-3′ | 22 | 30 | 499 |

TABLE BEB
Probe Name Ag1747

| Primers | Sequences | Length | Start Position | SEQ ID NO |
|---|---|---|---|---|
| Forward | 5′-gaggtgctttcaacaaaggaat-3′ | 22 | 108 | 500 |
| Probe | TET-5′-tcggttactcccttactgaatcccttca-3′-TAMRA | 28 | 64 | 501 |
| Reverse | 5′-gcttgtttcacttgctgatttc-3′ | 22 | 30 | 502 |

TABLE BEC
Panel 4D

| | Rel. Exp. (%) Ag1747, Run | | Rel. Exp. (%) Ag1747, Run |
|---|---|---|---|
| Tissue Name | 165808155 | Tissue Name | 165808155 |
| Secondary Th1 act | 0.0 | HUVEC IL-1beta | 0.0 |
| Secondary Th2 act | 0.0 | HUVEC IFN gamma | 0.0 |
| Secondary Tr1 act | 5.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 2.6 | Lung Microvascular EC none | 0.0 |
| Primary Th1 act | 0.0 | Lung Microvascular EC TNF alpha + | 0.0 |
| | | IL-1beta | |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 10.3 | Bronchial epithelium TNF alpha + | 0.0 |
| | | IL1beta | |
| Primary Th2 rest | 0.0 | Small airway epithelium none | 0.0 |
| Primary Tr1 rest | 0.0 | Small airway epithelium TNF alpha + | 0.0 |
| | | IL-1beta | |
| CD45RA CD4 lymphocyte | 0.0 | Coronery artery SMC rest | 0.0 |
| act | | | |
| CD45RO CD4 lymphocyte | 3.0 | Coronery artery SMC TNF alpha + | 0.0 |
| act | | IL-1beta | |
| CD8 lymphocyte act | 10.2 | Astrocytes rest | 0.0 |
| Secondary CD8 lymphocyte | 7.2 | Astrocytes TNF alpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 0.0 |
| | | PMA/ionomycin | |
| 2ry Th1/Th2/Tr1_anti-CD95 | 2.9 | CCD1106 (Keratinocytes) none | 0.0 |
| CH11 | | | |
| LAK cells rest | 4.1 | CCD1106 (Keratinocytes) | 0.0 |
| | | TNF alpha + IL-1beta | |
| LAK cells IL-2 | 0.0 | Liver cirrhosis | 100.0 |
| LAK cells IL-2 + IL-12 | 7.5 | Lupus kidney | 0.0 |
| LAK cells IL-2 + IFN gamma | 13.1 | NCI-H292 none | 0.0 |
| LAK cells IL-2 + IL-18 | 0.0 | NCI-H292 IL-4 | 0.0 |
| LAK cells PMA/ionomycin | 15.3 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 5.9 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 10.0 | Lung fibroblast TNF alpha + IL-1 | 0.0 |
| | | beta | |
| PBMC PHA-L | 6.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 8.2 | Dermal fibroblast CCD1070 rest | 0.0 |
| IL-4 | | | |
| EOL-1 dbcAMP | 0.0 | Dermal fibroblast CCD1070 TNF | 0.0 |
| | | alpha | |
| EOL-1 dbcAMP | 0.0 | Dermal fibroblast CCD1070 IL-1 | 0.0 |
| PMA/ionomycin | | beta | |
| Dendritic cells none | 4.2 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 12.4 |
| Monocytes rest | 0.0 | IBD Crohn's | 7.9 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag1703 Expression of the CG57425-01 gene is low/undetectable (CTs>35) across all of the samples on this panel due to a probable probe or chemistry failure (data not shown). Ag1747 Expression of this gene is low/undetectable (CTs>35) across all of the samples on this panel (data not shown).

Panel 2.2 Summary: Ag1703 Expression of the CG57425-01 gene is low/undetectable (CTs>35) across all of the samples on this panel due to a probable probe or chemistry failure (data not shown). Ag1747 Expression of this gene is low/undetectable (CTs>35) across all of the samples on this panel (data not shown).

Panel 4D Summary: Ag1703 Expression of CG57425-01 gene is low/undetectable (CTs>35) across all of the samples on this panel due to a probable probe or chemistry failure (data not shown). Ag1747 Low levels of expression of this gene are detected in a liver cirrhosis sample (CT=33.1). Furthermore, expression of this gene is not detected in normal liver in Panel 1.3D, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

NOV80

Expression of gene CG56766-01 was assessed using the primer-probe set Ag3013, described in Table BPA.

TABLE BPA
Probe Name Ag3013

| | | | Start | |
|---|---|---|---|---|
| Primers | Sequences | Length | Position | SEQ ID NO |
| Forward | 5′-tacactgttgtcacgccaataa-3′ | 22 | 888 | 503 |
| Probe | TET-5′-ccctttgatttactgcctgaggaaca-3′-TAMRA | 26 | 914 | 504 |
| Reverse | 5′-ttttcaaggcgtccttaaattc-3′ | 22 | 942 | 505 |

CNS_neurodegeneration_v1.0 Summary: Ag3013 Expression of the CG56766-01 gene is low/undetectable (CTs>35) across all of the samples on this panel (data not shown).

Panel 1.3D Summary: Ag3013 Expression of this gene is low/undetectable (CTs>35) across all of the samples on this panel (data not shown). The CG56766-01 gene encodes a G protein-coupled receptor (GPCR), a type of cell surface receptor involved in signal transduction. This gene product is most similar to members of the odorant receptor subfamily of GPCRs. Based on analogy to other odorant receptor genes, we predict that expression of this gene may be highest in nasal epithelium, a sample not represented on this panel.

Panel 4D Summary: Ag3013 Expression of the CG56766-01 gene is low/undetectable (CTs>35) across all of the samples on this panel (data not shown).

NOV81

Expression of NOV81A/CG57847-01 and NOV81B/CG57847-02 was assessed using the primer-probe set Ag3347, described in Table BQA. Results of the RTQ-PCR runs are shown in Table BQB. Please note that CG57847-02 represents a full-length physical clone of the CG57847-01 gene, validating the prediction of the gene sequence.

TABLE BQA
Probe Name Ag3347

| Primers | Sequences | Length | Start Position | SEQ ID NO |
|---|---|---|---|---|
| Forward | 5′-attgatttggtttggctacttg-3′ | 22 | 866 | 506 |
| Probe | TET-5′-aactctacatttaatccaatggtttatgca-3′-TAMRA | 30 | 888 | 507 |
| Reverse | 5′-accaaacagcatcatcttcagt-3′ | 22 | 944 | 508 |

TABLE BOB
General_screening_panel_v1.4

| | Rel. Exp. (%) Ag3347, Run | | Rel. Exp. (%) Ag3347, Run |
|---|---|---|---|
| Tissue Name | 215620630 | Tissue Name | 215620630 |
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI- | 0.0 |
| | | N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell carcinoma | 0.0 | Colon ca.* (SW480 met) | 0.0 |
| SCC-4 | | SW620 | |
| Testis Pool | 0.0 | Colon ca. HT29 | 0.0 |
| Prostate ca.* (bone met) | 2.3 | Colon ca. HCT-116 | 1.5 |
| PC-3 | | | |
| Prostate Pool | 0.0 | Colon ca. CaCo-2 | 1.8 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 7.4 | Colon ca. SW-48 | 0.0 |
| Ovarian ca. OVCAR-4 | 0.0 | Colon Pool | 4.1 |
| Ovarian ca. OVCAR-5 | 0.0 | Small Intestine Pool | 0.0 |
| Ovarian ca. IGROV-1 | 0.0 | Stomach Pool | 3.7 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 2.7 |
| Breast ca. MDA-MB-231 | 0.0 | Lymph Node Pool | 3.3 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 4.5 |
| Breast Pool | 0.0 | Thymus Pool | 0.0 |
| Trachea | 11.0 | CNS cancer (glio/astro) U87- | 0.0 |
| | | MG | |
| Lung | 1.7 | CNS cancer (glio/astro) U- | 4.0 |
| | | 118-MG | |
| Fetal Lung | 5.8 | CNS cancer (neuro;met) SK- | 0.0 |
| | | N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 75.3 | CNS cancer (glio) SNB-19 | 0.0 |
| Lung ca. SHP-77 | 100.0 | CNS cancer (glio) SF-295 | 0.0 |
| Lung ca. A549 | 0.0 | Brain (Amygdala) Pool | 1.9 |
| Lung ca. NCI-H526 | 0.0 | Brain (cerebellum) | 0.0 |
| Lung ca. NCI-H23 | 21.9 | Brain (fetal) | 0.0 |
| Lung ca. NCI-H460 | 8.2 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) Pool | 0.0 |
| Liver | 0.0 | Brain (Thalamus) Pool | 0.0 |
| Fetal Liver | 1.5 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 6.1 | Pituitary gland Pool | 0.0 |
| Renal ca. 786-0 | 0.0 | Salivary Gland | 4.7 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 5.3 |

CNS_neurodegeneration_v1.0 Summary: Ag3347 Results from one experiment with the CG57847-01 gene are not included. The amp plot indicates that there were experimental difficulties with this run.

General_screening_panel_v1.4 Summary: Ag3347 Expression of the CG57847-01 gene is restricted to two lung cancer cell lines. Therefore, expression of this gene may be used to distinguish lung cancer cell lines from the other samples on this panel. In addition, therapeutic modulation of the activity of this gene or its protein product, through the use of small molecule drugs, protein therapeutics or antibodies, might be beneficial in the treatment of lung cancer.

Panel 4D Summary: Ag3347 Expression of the CG57847-01 gene is low/undetectable (CTs>35) across all of the samples on this panel (data not shown).

NOV82

Expression of NOV82/CG57845-01 was assessed using the primer-probe set Ag3346, described in Table BRA. Results of the RTQ-PCR runs are shown in Table BRB.

Table BRA. Probe Name Ag3346

TABLE BRA
Probe Name Ag3346

| Primers | Sequences | Length | Start Position | SEQ ID NO |
|---|---|---|---|---|
| Forward | 5′-actcttcacagttgctgtgatg-3′ | 22 | 317 | 509 |
| Probe | TET-5′-cacttgtgcttcatctgcatcgacag-3′-TAMRA | 26 | 365 | 510 |
| Reverse | 5′-aggggatcagtaaccacaatgt-3′ | 22 | 393 | 511 |

TABLE BRB
General_screening_panel_v1.4

| | Rel. Exp. (%) Ag3346, Run | | Rel. Exp. (%) Ag3346, Run |
|---|---|---|---|
| Tissue Name | 215601952 | Tissue Name | 215601952 |
| Adipose | 0.0 | Renal ca. TK-10 | 0.0 |
| Melanoma* Hs688(A).T | 0.0 | Bladder | 4.7 |
| Melanoma* Hs688(B).T | 0.0 | Gastric ca. (liver met.) NCI- | 0.0 |
| | | N87 | |
| Melanoma* M14 | 0.0 | Gastric ca. KATO III | 0.0 |
| Melanoma* LOXIMVI | 4.5 | Colon ca. SW-948 | 0.0 |
| Melanoma* SK-MEL-5 | 0.0 | Colon ca. SW480 | 0.0 |
| Squamous cell carcinoma | 0.0 | Colon ca.* (SW480 met) | 0.0 |
| SCC-4 | | SW620 | |
| Testis Pool | 6.1 | Colon ca. HT29 | 0.0 |
| Prostate ca.* (bone met) | 0.0 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 8.4 |
| Ovarian ca. OVCAR-5 | 7.9 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 2.4 |
| Ovary | 0.0 | Fetal Heart | 0.0 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer (glio/astro) U87- | 0.0 |
| | | MG | |
| Lung | 0.0 | CNS cancer (glio/astro) U- | 6.0 |
| | | 118-MG | |
| Fetal Lung | 0.0 | CNS cancer (neuro;met) SK- | 0.0 |
| | | N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 23.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 100.0 | Brain (fetal) | 0.0 |
| Lung ca. NCI-H460 | 19.8 | Brain (Hippocampus) Pool | 8.7 |
| Lung ca. HOP-62 | 0.0 | Cerebral Cortex Pool | 11.5 |
| Lung ca. NCI-H522 | 0.0 | Brain (Substantia nigra) Pool | 0.0 |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 6.3 | Adrenal Gland | 0.0 |
| Fetal Kidney | 7.6 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 4.2 | Pancreatic ca. CAPAN2 | 0.0 |
| Renal ca. UO-31 | 0.0 | Pancreas Pool | 0.0 |

CNS_neurodegeneration_v1.0 Summary: Ag3346 Results from one experiment with the CG57845-01 gene are not included. The amp plot indicates that there were experimental difficulties with this run.

General_screening_panel_v1.4 Summary: Ag3346 Significant expression of the CG57845-01 gene is detected exclusively in a lung cancer cell line NCI-H23 sample (CT=33.7). Therefore, expression of this gene may be used to distinguish lung cancers from the other samples on this panel. Furthermore, therapeutic modulation of the activity of the GPCR encoded by this gene may be beneficial in the treatment of lung cancer.

Panel 4D Summary: Ag3346 Expression of the CG57845-01 gene is low/undetectable (CTs>35) across all of the samples on this panel (data not shown).

NOV83

Expression of NOV83 CG57843-01 was assessed using the primer-probe set Ag3345, described in Table BSA. Results of the RTQ-PCR runs are shown in Table BSB.

TABLE BSA
Probe Name Ag3345

| Primers | Sequences | Length | Start Position | SEQ ID NO |
|---|---|---|---|---|
| Forward | 5′-catctccatcgacaggtacatt-3′ | 22 | 379 | 512 |
| Probe | TET-5′-ccctggtctatcctaccaagttcaccg-3′-TAMRA | 27 | 414 | 513 |
| Reverse | 5′-gatgcaaattcctgacacagat-3′ | 22 | 442 | 514 |

TABLE BSB
General_screening_panel_v1.4

| | Rel. Exp. (%) Ag3345, Run | | Rel. Exp. (%) Ag3345, Run |
|---|---|---|---|
| Tissue Name | 215773854 | Tissue Name | 215773854 |
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI- | 0.0 |
| | | N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell carcinoma | 0.0 | Colon ca.* (SW480 met) | 0.0 |
| SCC-4 | | SW620 | |
| Testis Pool | 0.0 | Colon ca. HT29 | 0.0 |
| Prostate ca.* (bone met) | 0.0 | Colon ca. HCT-116 | 0.0 |
| PC-3 | | | |
| Prostate Pool | 0.0 | Colon ca. CaCo-2 | 0.0 |
| Placenta | 0.0 | Colon cancer tissue | 0.0 |
| Uterus Pool | 100.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.1 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.1 |
| Trachea | 0.0 | CNS cancer (glio/astro) U87- | 0.0 |
| | | MG | |
| Lung | 0.0 | CNS cancer (glio/astro) U- | 0.1 |
| | | 118-MG | |
| Fetal Lung | 0.0 | CNS cancer (neuro;met) SK- | 0.0 |
| | | N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.2 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 0.1 |
| Lung ca. NCI-H526 | 0.0 | Brain (cerebellum) | 0.0 |
| Lung ca. NCI-H23 | 0.8 | Brain (fetal) | 0.0 |
| Lung ca. NCI-H460 | 0.3 | Brain (Hippocampus) Pool | 0.0 |
| Lung ca. HOP-62 | 0.0 | Cerebral Cortex Pool | 0.1 |
| Lung ca. NCI-H522 | 0.0 | Brain (Substantia nigra) Pool | 0.1 |
| Liver | 0.0 | Brain (Thalamus) Pool | 0.1 |
| Fetal Liver | 3.1 | Brain (whole) | 0.1 |
| Liver ca. HepG2 | 0.0 | Spinal Cord Pool | 0.0 |
| Kidney Pool | 0.1 | Adrenal Gland | 0.0 |
| Fetal Kidney | 0.2 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.1 | Pancreatic ca. CAPAN2 | 0.0 |
| Renal ca. UO-31 | 0.0 | Pancreas Pool | 0.0 |

CNS_neurodegeneration_v1.0 Summary: Ag3345 Expression of the CG57843-01 gene is low/undetectable (CTs>35) across all of the samples on this panel (data not shown).

General_screening_panel_v1.4 Summary: Ag3345 Expression of the CG57843-01 gene is highest in a sample derived from normal uterus (CT=26.6). Thus, expression of this gene can be used to distinguish uterus from the other samples on this panel. Interestingly, expression of this gene is also detected in fetal liver (CT=31) at much higher levels than in adult liver (CT=40), suggesting that expression of this gene can be used to distinguish fetal from adult liver.

Panel 4D Summary: Ag3345 Expression of the CG57843-01 gene is low/undetectable (CTs>34.5) across all of the samples on this panel (data not shown).

NOV84

Expression of NOV84a, NOV84b, and NOV84c (CG57841-01, CG57841-02, and CG57837-01) was assessed using the primer-probe sets Ag3342 and Ag3344, described in Tables BTA and BTB. Results of the RTQ-PCR runs are shown in Table BTC. Please note that CG57841-02 represents a full-length physical clone of the CG57841-01 gene, validating the prediction of the gene sequence.

TABLE BTA
Probe Name Ag3342

| Primers | Sequences | Length | Start Position | SEQ ID NO |
|---|---|---|---|---|
| Forward | 5′-ttacctcctgaccataatggaa-3′ | 22 | 111 | 515 |
| Probe | TET-5′-aacctgatgctgctgctcatgatcag-3′-TAMRA | 26 | 133 | 516 |
| Reverse | 5′-tacatgggcttatggagacaag-3′ | 22 | 167 | 517 |

TABLE BTB
Probe Name Ag3344

| Primers | Sequences | Length | Start Position | SEQ ID NO |
|---|---|---|---|---|
| Forward | 5′-ttacctcctgaccataatggaa-3′ | 22 | 111 | 518 |
| Probe | TET-5′-aacctgatgctgctgctcatgatcag-3′-TAMRA | 26 | 133 | 519 |
| Reverse | 5′-tacatgggcttatggagacaag-3′ | 22 | 167 | 520 |

TABLE BTC
General_screening_panel_v1.4

| | Rel. Exp. (%) Ag3342, Run | | Rel. Exp. (%) Ag3342, Run |
|---|---|---|---|
| Tissue Name | 215773812 | Tissue Name | 215773812 |
| Adipose | 2.4 | Renal ca. TK-10 | 5.3 |
| Melanoma* Hs688(A).T | 0.0 | Bladder | 8.3 |
| Melanoma* Hs688(B).T | 0.0 | Gastric ca. (liver met.) NCI- | 40.9 |
| | | N87 | |
| Melanoma* M14 | 0.0 | Gastric ca. KATO III | 0.0 |
| Melanoma* LOXIMVI | 4.6 | Colon ca. SW-948 | 0.0 |
| Melanoma* SK-MEL-5 | 0.0 | Colon ca. SW480 | 7.8 |
| Squamous cell carcinoma | 0.0 | Colon ca.* (SW480 met) | 5.1 |
| SCC-4 | | SW620 | |
| Testis Pool | 27.4 | Colon ca. HT29 | 0.0 |
| Prostate ca.* (bone met) | 0.0 | Colon ca. HCT-116 | 2.3 |
| PC-3 | | | |
| Prostate Pool | 3.4 | Colon ca. CaCo-2 | 16.4 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 4.8 | Colon Pool | 18.6 |
| Ovarian ca. OVCAR-5 | 0.0 | Small Intestine Pool | 15.4 |
| Ovarian ca. IGROV-1 | 4.3 | Stomach Pool | 0.0 |
| Ovarian ca. OVCAR-8 | 0.0 | Bone Marrow Pool | 9.2 |
| Ovary | 21.0 | Fetal Heart | 0.0 |
| Breast ca. MCF-7 | 7.5 | Heart Pool | 4.4 |
| Breast ca. MDA-MB-231 | 0.0 | Lymph Node Pool | 21.5 |
| Breast ca. BT 549 | 11.0 | Fetal Skeletal Muscle | 0.0 |
| Breast ca. T47D | 0.0 | Skeletal Muscle Pool | 3.0 |
| Breast ca. MDA-N | 0.0 | Spleen Pool | 5.6 |
| Breast Pool | 11.4 | Thymus Pool | 0.0 |
| Trachea | 0.0 | CNS cancer (glio/astro) U87- | 4.2 |
| | | MG | |
| Lung | 16.8 | CNS cancer (glio/astro) U- | 0.0 |
| | | 118-MG | |
| Fetal Lung | 0.0 | CNS cancer (neuro;met) SK- | 0.0 |
| | | N-AS | |
| Lung ca. NCI-N417 | 0.0 | CNS cancer (astro) SF-539 | 0.0 |
| Lung ca. LX-1 | 3.1 | CNS cancer (astro) SNB-75 | 0.0 |
| Lung ca. NCI-H146 | 12.5 | CNS cancer (glio) SNB-19 | 0.0 |
| Lung ca. SHP-77 | 0.0 | CNS cancer (glio) SF-295 | 5.4 |
| Lung ca. A549 | 0.0 | Brain (Amygdala) Pool | 0.0 |
| Lung ca. NCI-H526 | 0.0 | Brain (cerebellum) | 5.9 |
| Lung ca. NCI-H23 | 100.0 | Brain (fetal) | 0.0 |
| Lung ca. NCI-H460 | 8.3 | Brain (Hippocampus) Pool | 15.3 |
| Lung ca. HOP-62 | 0.0 | Cerebral Cortex Pool | 8.1 |
| Lung ca. NCI-H522 | 0.0 | Brain (Substantia nigra) Pool | 7.7 |
| Liver | 0.0 | Brain (Thalamus) Pool | 11.0 |
| Fetal Liver | 0.0 | Brain (whole) | 0.0 |
| Liver ca. HepG2 | 0.0 | Spinal Cord Pool | 16.4 |
| Kidney Pool | 23.8 | Adrenal Gland | 4.6 |
| Fetal Kidney | 15.7 | Pituitary gland Pool | 0.0 |
| Renal ca. 786-0 | 4.7 | Salivary Gland | 4.8 |
| Renal ca. A498 | 0.0 | Thyroid (female) | 0.0 |
| Renal ca. ACHN | 0.0 | Pancreatic ca. CAPAN2 | 9.8 |
| Renal ca. UO-31 | 0.0 | Pancreas Pool | 13.7 |

CNS_neurodegeneration_v1.0 Summary: Ag3342/Ag3344 Expression of the CG57841-01 gene is low/undetectable (CTs>34.5) across all of the samples on this panel (data not shown).

General_screening_panel_v1.4 Summary: Ag3342 Significant expression of the CG57841-01 gene is seen exclusively in a lung cancer NCI-H23 sample (CT=34.1). Therefore, expression of this gene may be used to distinguish this sample from the other samples on this panel. Furthermore, therapeutic modulation of the activity of the GPCR encoded by this gene may be beneficial in the treatment of lung cancer. Ag3344 Results from one experiment with the CG57841-01 gene are not included. The amp plot indicates that there were experimental difficulties with this run.

Panel 4D Summary: Ag3342/Ag3344 Expression of this gene is low/undetectable (CTs>35) across all of the samples on this panel (data not shown).

NOV85

Expression of NOV85 CG57839-01 was assessed using the primer-probe sets Ag2308 and Ag3343, described in Tables BUA and BUB. Results of the RTQ-PCR runs are shown in Tables BUC, and BUD.

TABLE BUA
Probe Name Ag2308

| Primers | Sequences | Length | Start Position | SEQ ID NO |
|---|---|---|---|---|
| Forward | 5′-taccgatcatagcacatcatca-3′ | 22 | 323 | 521 |
| Probe | TET-5′-tcagacactctgtaatagcaaacgcca-3′-TAMRA | 27 | 290 | 522 |
| Reverse | 5′-tgctccttgcatacttcagact-3′ | 22 | 258 | 523 |

TABLE BUB
Probe Name Ag3343

| Primers | Sequences | Length | Start Position | SEQ ID NO |
|---|---|---|---|---|
| Forward | 5′-ctttccctgttttacagccttt-3′ | 22 | 826 | 524 |
| Probe | TET-5′-cccctcatctacagccttaatgcaga-3′-TAMRA | 26 | 865 | 525 |
| Reverse | 5′-ctctctttagagcccctttcac-3′ | 22 | 892 | 526 |

TABLE BUC
CNS_neurodegeneration_v1.0

| | Rel. Exp. (%) Ag2308, Run | | Rel. Exp. (%) Ag2308, Run |
|---|---|---|---|
| Tissue Name | 207970871 | Tissue Name | 207970871 |
| AD 1 Hippo | 0.0 | Control (Path) 3 Temporal | 0.0 |
| | | Ctx | |
| AD 2 Hippo | 9.8 | Control (Path) 4 Temporal | 10.7 |
| | | Ctx | |
| AD 3 Hippo | 0.0 | AD 1 Occipital Ctx | 24.0 |
| AD 4 Hippo | 18.2 | AD 2 Occipital Ctx | 0.0 |
| | | (Missing) | |
| AD 5 Hippo | 16.6 | AD 3 Occipital Ctx | 0.0 |
| AD 6 Hippo | 81.8 | AD 4 Occipital Ctx | 1.2 |
| Control 2 Hippo | 0.0 | AD 5 Occipital Ctx | 1.8 |
| Control 4 Hippo | 38.7 | AD 6 Occipital Ctx | 1.9 |
| Control (Path) 3 Hippo | 1.2 | Control 1 Occipital Ctx | 17.6 |
| AD 1 Temporal Ctx | 16.4 | Control 2 Occipital Ctx | 0.0 |
| AD 2 Temporal Ctx | 14.6 | Control 3 Occipital Ctx | 17.2 |
| AD 3 Temporal Ctx | 0.0 | Control 4 Occipital Ctx | 30.8 |
| AD 4 Temporal Ctx | 20.4 | Control (Path) 1 Occipital | 0.0 |
| | | Ctx | |
| AD 5 Inf Temporal Ctx | 15.1 | Control (Path) 2 Occipital | 11.7 |
| | | Ctx | |
| AD 5 Sup Temporal Ctx | 27.9 | Control (Path) 3 Occipital | 0.0 |
| | | Ctx | |
| AD 6 Inf Temporal Ctx | 13.0 | Control (Path) 4 Occipital | 16.7 |
| | | Ctx | |
| AD 6 Sup Temporal Ctx | 1.6 | Control 1 Parietal Ctx | 0.0 |
| Control 1 Temporal Ctx | 0.0 | Control 2 Parietal Ctx | 0.0 |
| Control 2 Temporal Ctx | 1.2 | Control 3 Parietal Ctx | 0.0 |
| Control 3 Temporal Ctx | 100.0 | Control (Path) 1 Parietal | 4.0 |
| | | Ctx | |
| Control 3 Temporal Ctx | 38.7 | Control (Path) 2 Parietal | 0.0 |
| | | Ctx | |
| Control (Path) 1 Temporal | 16.2 | Control (Path) 3 Parietal | 10.3 |
| Ctx | | Ctx | |
| Control (Path) 2 Temporal | 0.0 | Control (Path) 4 Parietal | 31.0 |
| Ctx | | Ctx | |

TABLE BUD
Panel 4D

| | Rel. Exp. (%) Ag2308, Run | | Rel. Exp. (%) Ag2308, Run |
|---|---|---|---|
| Tissue Name | 158927487 | Tissue Name | 158927487 |
| Secondary Th1 act | 0.0 | HUVEC IL-1beta | 0.0 |
| Secondary Th2 act | 0.0 | HUVEC IFN gamma | 17.4 |
| Secondary Tr1 act | 0.0 | HUVEC TNF alpha + IFN gamma | 0.0 |
| Secondary Th1 rest | 17.2 | HUVEC TNF alpha + IL4 | 0.0 |
| Secondary Th2 rest | 0.0 | HUVEC IL-11 | 4.4 |
| Secondary Tr1 rest | 0.0 | Lung Microvascular EC none | 0.0 |
| Primary Th1 act | 11.3 | Lung Microvascular EC TNF alpha + | 0.0 |
| | | IL-1beta | |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 24.0 | Bronchial epithelium TNF alpha + | 0.0 |
| | | IL1beta | |
| Primary Th2 rest | 14.5 | Small airway epithelium none | 0.0 |
| Primary Tr1 rest | 0.0 | Small airway epithelium TNF alpha + | 15.1 |
| | | IL-1beta | |
| CD45RA CD4 lymphocyte | 0.0 | Coronery artery SMC rest | 0.0 |
| act | | | |
| CD45RO CD4 lymphocyte | 6.0 | Coronery artery SMC TNF alpha + | 0.0 |
| act | | IL-1beta | |
| CD8 lymphocyte act | 0.0 | Astrocytes rest | 0.0 |
| Secondary CD8 lymphocyte | 0.0 | Astrocytes TNF alpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 29.1 |
| LAK cells IL-2 + IL-12 | 0.0 | Lupus kidney | 9.9 |
| LAK cells IL-2 + IFN gamma | 21.8 | NCI-H292 none | 26.6 |
| LAK cells IL-2 + IL-18 | 0.0 | NCI-H292 IL-4 | 28.7 |
| LAK cells PMA/ionomycin | 0.0 | NCI-H292 IL-9 | 37.9 |
| NK Cells IL-2 rest | 0.0 | NCI-H292 IL-13 | 0.0 |
| Two Way MLR 3 day | 10.6 | NCI-H292 IFN gamma | 13.3 |
| Two Way MLR 5 day | 21.5 | HPAEC none | 0.0 |
| Two Way MLR 7 day | 0.0 | HPAEC TNF alpha + IL-1beta | 0.0 |
| PBMC rest | 0.0 | Lung fibroblast none | 11.3 |
| PBMC PWM | 0.0 | Lung fibroblast TNF alpha + IL-1 | 0.0 |
| | | beta | |
| PBMC PHA-L | 14.6 | Lung fibroblast IL-4 | 18.6 |
| Ramos (B cell) none | 0.0 | Lung fibroblast IL-9 | 0.0 |
| Ramos (B cell) ionomycin | 0.0 | Lung fibroblast IL-13 | 15.9 |
| B lymphocytes PWM | 0.0 | Lung fibroblast IFN gamma | 22.2 |
| B lymphocytes CD40L and | 0.0 | Dermal fibroblast CCD1070 rest | 0.0 |
| IL-4 | | | |
| EOL-1 dbcAMP | 0.0 | Dermal fibroblast CCD1070 TNF | 6.7 |
| | | alpha | |
| EOL-1 dbcAMP | 0.0 | Dermal fibroblast CCD1070 IL-1 | 0.0 |
| PMA/ionomycin | | beta | |
| Dendritic cells none | 0.0 | Dermal fibroblast IFN gamma | 0.0 |
| Dendritic cells LPS | 0.0 | Dermal fibroblast IL-4 | 27.5 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 8.7 |
| Macrophages rest | 7.5 | Lung | 47.0 |
| Macrophages LPS | 0.0 | Thymus | 100.0 |
| HUVEC none | 0.0 | Kidney | 5.8 |
| HUVEC starved | 0.0 | | |

CNS_neurodegeneration_v1.0 Summary: Ag2308 The CG57839-01 gene is expressed at low levels in the brains of both normal and Alzheimer's disease patients and encodes a putative GPCR (CTs=33-35). Several neurotransmitter receptors are GPCRs, including the dopamine receptor family, the serotonin receptor family, the GABAB receptor, muscarinic acetylcholine receptors, and others; thus this GPCR may represent a novel neurotransmitter receptor. Targeting various neurotransmitter receptors (dopamine, serotonin) has proven to be an effective therapy in psychiatric illnesses such as schizophrenia, bipolar disorder, and depression. Furthermore, the cerebral cortex and hippocampus are regions of the brain that are known to be involved in Alzheimer's disease, seizure disorders, and in the normal process of memory formation. Therefore, therapeutic modulation of this gene or its protein product may be beneficial in the treatment of one or more of these diseases, as may stimulation and/or blockade of the receptor coded for by the gene. Ag3343 Expression of this gene is low/undetectable (CTs>35) across all of the samples on this panel (data not shown).

References:

1. El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol 2001 September;134(1):68-77

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

2. Blier P. Pharmacology of rapid-onset antidepressant treatment strategies. Clin Psychiatry 2001;62 Suppl 15:12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSRI treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

3. Tranquillini M E, Reggiani A. Glycine-site antagonists and stroke. Expert Opin Investig Drugs 1999 November;8(11):1837-1848

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

4. Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 1.3D Summary: Ag2308 Expression of the CG57839-01 gene is low/undetectable (CT>35) in all samples shown in this Panel (data not shown).

Panel 4D Summary: Ag2308 Expression of the CG57839-01 gene is detected in the thymus (CT=33.3) and lung (CT=34.4). Thus, expression of this gene could be used as a marker to detect the presence of thymus or lung tissue. The putative GPCR encoded for by this gene may also play an important role in the normal homeostasis of these tissues. Therefore, therapeutics designed with this gene product could be important for maintaining or restoring normal function to these organs during inflammation. Ag3343 Expression of this gene is low/undetectable (CTs>35) across all of the samples on this panel (data not shown).

NOV87

Expression of NOV87 CG56763-01 was assessed using the primer-probe set Ag3012, described in Table BVA. Results of the RTQ-PCR runs are shown in Tables BVB, and BVC.

TABLE BVA
Probe Name Ag3012

| Primers | Sequences | Length | Start Position | SEQ ID NO |
|---|---|---|---|---|
| Forward | 5′-ctctttgtcctggtggagaac-3′ | 21 | 162 | 527 |
| Probe | TET-5′-acctccctccacaggcccatgtacta-3′-TAMRA | 26 | 213 | 528 |
| Reverse | 5′-gaaagacatggagctcagaaag-3′ | 22 | 239 | 529 |

TABLE BVB
Panel 1.3D

| | Rel. Exp. (%) Ag3012, Run | | Rel. Exp. (%) Ag3012, Run |
|---|---|---|---|
| Tissue Name | 167810404 | Tissue Name | 167810404 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 1.6 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 2.8 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 12.6 | Liver ca. (hepatoblast) | 0.0 |
| | | HepG2 | |
| Brain (cerebellum) | 0.0 | Lung | 0.0 |
| Brain (hippocampus) | 5.5 | Lung (fetal) | 0.0 |
| Brain (substantia nigra) | 3.0 | Lung ca. (small cell) LX-1 | 1.4 |
| Brain (thalamus) | 3.1 | Lung ca. (small cell) NCI- | 0.0 |
| | | H69 | |
| Cerebral Cortex | 0.0 | Lung ca. (s.cell var.) SHP- | 2.4 |
| | | 77 | |
| Spinal cord | 100.0 | Lung ca. (large cell)NCI- | 0.0 |
| | | H460 | |
| glio/astro U87-MG | 0.0 | Lung ca. (non-sm. cell) | 0.0 |
| | | A549 | |
| glio/astro U-118-MG | 0.0 | Lung ca. (non-s.cell) NCI- | 0.0 |
| | | H23 | |
| astrocytoma SW1783 | 0.0 | Lung ca. (non-s.cell) HOP- | 0.0 |
| | | 62 | |
| neuro*; met SK-N-AS | 0.0 | Lung ca. (non-s.cl) NCI- | 0.0 |
| | | H522 | |
| astrocytoma SF-539 | 0.0 | Lung ca. (squam.) SW 900 | 0.0 |
| astrocytoma SNB-75 | 0.0 | Lung ca. (squam.) NCI- | 0.0 |
| | | H596 | |
| glioma SNB-19 | 3.2 | Mammary gland | 2.9 |
| glioma U251 | 0.0 | Breast ca.* (pl.ef) MCF-7 | 3.5 |
| glioma SF-295 | 0.0 | Breast ca.* (pl.ef) MDA- | 0.0 |
| | | MB-231 | |
| Heart (fetal) | 0.0 | Breast ca.* (pl.ef) T47D | 0.0 |
| Heart | 2.6 | Breast ca. BT-549 | 0.0 |
| Skeletal muscle (fetal) | 12.5 | Breast ca. MDA-N | 0.0 |
| Skeletal muscle | 0.0 | Ovary | 0.0 |
| Bone marrow | 0.0 | Ovarian ca. OVCAR-3 | 3.0 |
| Thymus | 9.5 | Ovarian ca. OVCAR-4 | 0.0 |
| Spleen | 0.0 | Ovarian ca. OVCAR-5 | 3.5 |
| Lymph node | 0.0 | Ovarian ca. OVCAR-8 | 0.0 |
| Colorectal | 4.3 | Ovarian ca. IGROV-1 | 0.0 |
| Stomach | 0.0 | Ovarian ca.* (ascites) SK- | 0.0 |
| | | OV-3 | |
| Small intestine | 0.0 | Uterus | 0.0 |
| Colon ca. SW480 | 0.0 | Placenta | 0.0 |
| Colon ca.* SW620(SW480 | 0.0 | Prostate | 0.0 |
| met) | | | |
| Colon ca. HT29 | 0.0 | Prostate ca.* (bone met)PC-3 | 0.0 |
| Colon ca. HCT-116 | 0.0 | Testis | 35.4 |
| Colon ca. CaCo-2 | 0.0 | Melanoma Hs688(A).T | 0.0 |
| Colon ca. tissue (ODO3866) | 0.0 | Melanoma* (met) | 0.0 |
| | | Hs688(B).T | |
| Colon ca. HCC-2998 | 6.0 | Melanoma UACC-62 | 0.0 |
| Gastric ca.* (liver met) NCI- | 0.0 | Melanoma M14 | 0.0 |
| N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE BVC
Panel 4D

| | Rel. Exp. (%) Ag3012, Run | | Rel. Exp. (%) Ag3012, Run |
|---|---|---|---|
| Tissue Name | 164404080 | Tissue Name | 164404080 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + | 0.0 |
| | | IL-1beta | |
| Primary Th2 act | 0.0 | Microvascular Dermal EC none | 0.0 |
| Primary Tr1 act | 0.0 | Microvasular Dermal EC | 0.0 |
| | | TNF alpha + IL-1beta | |
| Primary Th1 rest | 0.0 | Bronchial epithelium TNF alpha + | 0.0 |
| | | IL1beta | |
| Primary Th2 rest | 8.0 | Small airway epithelium none | 0.0 |
| Primary Tr1 rest | 0.0 | Small airway epithelium TNF alpha + | 0.0 |
| | | IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNF alpha + | 0.0 |
| act | | IL-1beta | |
| CD8 lymphocyte act | 0.0 | Astrocytes rest | 7.2 |
| Secondary CD8 lymphocyte | 7.9 | Astrocytes TNF alpha + IL-1beta | 0.0 |
| rest | | | |
| Secondary CD8 lymphocyte | 0.0 | KU-812 (Basophil) rest | 0.0 |
| act | | | |
| CD4 lymphocyte none | 10.2 | KU-812 (Basophil) | 7.7 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 10.7 | CCD1106 (Keratinocytes) | 0.0 |
| | | TNF alpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN gamma | 9.4 | NCI-H292 none | 0.0 |
| LAK cells IL-2 + IL-18 | 14.1 | NCI-H292 IL-4 | 0.0 |
| LAK cells PMA/ionomycin | 0.0 | NCI-H292 IL-9 | 18.9 |
| NK Cells IL-2 rest | 0.0 | NCI-H292 IL-13 | 0.0 |
| Two Way MLR 3 day | 4.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1beta | 0.0 |
| PBMC rest | 0.0 | Lung fibroblast none | 0.0 |
| PBMC PWM | 8.1 | Lung fibroblast TNF alpha + IL-1 | 0.0 |
| | | beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and | 19.6 | Dermal fibroblast CCD1070 rest | 0.0 |
| IL-4 | | | |
| EOL-1 dbcAMP | 16.7 | Dermal fibroblast CCD1070 TNF | 0.0 |
| | | alpha | |
| EOL-1 dbcAMP | 0.0 | Dermal fibroblast CCD1070 IL-1 | 0.0 |
| PMA/ionomycin | | beta | |
| Dendritic cells none | 0.0 | Dermal fibroblast IFN gamma | 0.0 |
| Dendritic cells LPS | 4.5 | Dermal fibroblast IL-4 | 0.0 |
| Dendritic cells anti-CD40 | 0.0 | IBD Colitis 2 | 0.0 |
| Monocytes rest | 0.0 | IBD Crohn's | 57.0 |
| Monocytes LPS | 0.0 | Colon | 56.6 |
| Macrophages rest | 0.0 | Lung | 65.5 |
| Macrophages LPS | 0.0 | Thymus | 14.9 |
| HUVEC none | 0.0 | Kidney | 0.0 |
| HUVEC starved | 0.0 | | |

CNS_neurodegeneration_v1.0 Summary: Ag3012 Expression of the CG56763-01 gene is low/undetectable (CTs>34.5) across all of the samples on this panel (data not shown).

Panel 1.3D Summary: Ag3012 The CG56763-01 gene is expressed at low levels in the samples derived from spinal cord (CT=32.1) and testis (CT=33.6). Thus, the expression of this gene could be used to distinguish these samples from the other samples in the panel. Furthermore, therapeutic modulation of the expression or function of this gene may be effective in the treatment of CNS disorders, fertility and hypogonadism.

Panel 4D Summary: Ag3012 The CG56763-01 gene is expressed at low levels in a sample derived from liver cirrhosis (CT=33.4). In addition, expression of this gene is not detected in normal liver in Panel 1.3D, suggesting that its expression is unique to liver cirrhosis. This gene encodes a putative GPCR; therefore, antibodies or small molecule therapeutics could reduce or inhibit fibrosis that occurs in liver cirrhosis. This gene is also expressed at low levels in the lung and colon.

NOV88

Expression of NOV88/CG56753-01 was assessed using the primer-probe sets Ag2875 and Ag3010, described in Tables BWA and BWB. Results of the RTQ-PCR runs are shown in Table BWC.

TABLE BWA
Probe Name Ag2875

| Primers | Sequences | Length | Start Position | SEQ ID NO |
|---|---|---|---|---|
| Forward | 5′-gaacatcatctcctaccctgaa-3′ | 22 | 267 | 530 |
| Probe | TET-5′-tgcatgactcagctctacttcttcctcg-3′-TAMRA | 28 | 289 | 531 |
| Reverse | 5′-atgtgacactctgcaatagcaa-3′ | 22 | 320 | 532 |

TABLE BWB
Probe Name Ag3010

| Primers | Sequences | Length | Start Position | SEQ ID NO |
|---|---|---|---|---|
| Forward | 5′-ggggaaagtatcctctgtgttt-3′ | 22 | 810 | 533 |
| Probe | TET-5′-ttattgtgcccatgttgaaccctctg-3′-TAMRA | 26 | 839 | 534 |
| Reverse | 5′-cagggaaacatggacatcttta-3′ | 22 | 882 | 535 |

TABLE BWC
Panel 4D

| | Rel. Exp. (%) Ag2875, Run | | Rel. Exp. (%) Ag2875, Run |
|---|---|---|---|
| Tissue Name | 164311029 | Tissue Name | 164311029 |
| Secondary Th1 act | 0.0 | HUVEC IL-1beta | 0.0 |
| Secondary Th2 act | 0.0 | HUVEC IFN gamma | 3.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + | 0.0 |
| | | IL-1beta | |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 0.0 | Bronchial epithelium TNF alpha + | 0.0 |
| | | IL1beta | |
| Primary Th2 rest | 0.0 | Small airway epithelium none | 0.0 |
| Primary Tr1 rest | 0.0 | Small airway epithelium TNF alpha + | 0.0 |
| | | IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNF alpha + | 0.0 |
| act | | IL-1beta | |
| CD8 lymphocyte act | 0.0 | Astrocytes rest | 0.0 |
| Secondary CD8 lymphocyte | 0.0 | Astrocytes TNF alpha + IL-1beta | 0.0 |
| rest | | | |
| Secondary CD8 lymphocyte | 0.0 | KU-812 (Basophil) rest | 0.0 |
| act | | | |
| CD4 lymphocyte none | 0.0 | KU-812 (Basophil) | 24.0 |
| | | PMA/ionomycin | |
| 2ry Th1/Th2/Tr1_anti-CD95 | 5.1 | CCD1106 (Keratinocytes) none | 0.0 |
| CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 72.2 |
| LAK cells IL-2 + IL-12 | 0.0 | Lupus kidney | 0.0 |
| LAK cells IL-2 + IFN gamma | 0.0 | NCI-H292 none | 20.7 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1beta | 1.4 |
| PBMC rest | 0.0 | Lung fibroblast none | 0.0 |
| PBMC PWM | 0.0 | Lung fibroblast TNF alpha + IL-1 | 0.0 |
| | | beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 3.2 |
| B lymphocytes CD40L and | 0.0 | Dermal fibroblast CCD1070 rest | 0.0 |
| IL-4 | | | |
| EOL-1 dbcAMP | 3.1 | Dermal fibroblast CCD1070 TNF | 0.0 |
| | | alpha | |
| EOL-1 dbcAMP | 0.0 | Dermal fibroblast CCD1070 IL-1 | 0.0 |
| PMA/ionomycin | | beta | |
| Dendritic cells none | 33.2 | Dermal fibroblast IFN gamma | 0.0 |
| Dendritic cells LPS | 2.4 | Dermal fibroblast IL-4 | 1.9 |
| Dendritic cells anti-CD40 | 100.0 | IBD Colitis 2 | 16.3 |
| Monocytes rest | 0.0 | IBD Crohn's | 6.7 |
| Monocytes LPS | 0.0 | Colon | 0.0 |
| Macrophages rest | 44.1 | Lung | 21.8 |
| Macrophages LPS | 0.0 | Thymus | 0.0 |
| HUVEC none | 3.1 | Kidney | 0.0 |
| HUVEC starved | 0.0 | | |

Panel 1.3D Summary: Ag2875/Ag3010 Results from two experiments with the CG56753-01 gene are not included. The amp plots indicate that there were experimental difficulties with these runs.

Panel 2.2 Summary: Ag2875 Expression of this gene is low/undetectable (CTs>35) across all of the samples on this panel (data not shown).

Panel 4D Summary: Ag2875 Highest expression of the CG56753-01 gene is in anti-CD40 treated dendritic cells (CT=33.2), with much lower expression in untreated dendritic cells. Thus, this gene product may be important in dendritic cell activation.

Significant expression of this gene is also seen in liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis. In addition, significant expression of this gene is seen in resting macrophages. The putative GPCR encoded for by this transcript may therefore be important in macrophage detection of chemokine gradients and trafficking into specific sites within a tissue and in activation. Antibody or protein therapeutics designed against the protein encoded for by this transcript could reduce or inhibit inflammation in asthma, emphysema, allergy, psoriasis, arthritis, or any other condition in which macrophage localization/activation is important.

Ag3010 Expression of this gene is low/undetectable (CTs>35) across all of the samples on this panel (data not shown).

NOV90

Expression of NOV90 CG57676-01 was assessed using the primer-probe set Gpcr45, described in Table BXA.

TABLE BXA
Probe Name Gpcr45

| Primers | Sequences | Length | Start Position | SEQ ID NO |
|---|---|---|---|---|
| Forward | 5′-tgctattgcccaggctgtact-3′ | 21 | 676 | 536 |
| Probe | TET-5′-aggatgcagtcaaccactgggcttcag-3′-TAMRA | 27 | 698 | 537 |
| Reverse | 5′-tgagctccacatgttccaaatactt-3′ | 25 | 726 | 538 |

Panel 1.1 Summary: Gpcr45 The results from two experiments with the same probe and primer set do not correlate and no conclusions can be drawn from this data (data not shown).

NOV91

Expression of NOV91/CG57678-01 was assessed using the primer-probe sets Ag2497, Ag3306 and Gpcr22, described in Tables BYA, BYB and BYC. Results of the RTQ-PCR runs are shown in Tables BYD, and BYE

TABLE BYA
Probe Name Ag2497

| Primers | Sequences | Length | Start Position | SEQ ID NO |
|---|---|---|---|---|
| Forward | 5′-gtccttgaaggatccaaaactc-3′ | 22 | 162 | 539 |
| Probe | TET-5′-tttctttctttccaacctttccttgg-3′-TAMRA | 26 | 198 | 540 |
| Reverse | 5′-ctgctggtcaaacagaggtcta-3′ | 22 | 224 | 541 |

TABLE BYB
Probe Name Ag3306

| Primers | Sequences | Length | Start Position | SEQ ID NO |
|---|---|---|---|---|
| Forward | 5′-gggcaaatttctcactcttttc-3′ | 22 | 828 | 542 |
| Probe | TET-5′-ccccaactcttaatcccctcatctaca-3′-TAMRA | 27 | 863 | 543 |
| Reverse | 5′-ccctttacctccttgttcctta-3′ | 22 | 893 | 544 |

TABLE BYC
Probe Name Gpcr22

| Primers | Sequences | Length | Start Position | SEQ ID NO |
|---|---|---|---|---|
| Forward | 5′-aattcagatgtctatcgccagtgtt-3′ | 25 | 606 | 545 |
| Probe | TET-5′-tcctcctggtgatgcccttgatcattatc-3′-TAMRA | 29 | 632 | 546 |
| Reverse | 5′-ttagcaatagcaccagaagaggaaa-3′ | 25 | 662 | 547 |

TABLE BYD
CNS_neurodegeneration_v1.0

| | Rel. Exp. (%) Ag2497, Run | | Rel. Exp. (%) Ag2497, Run |
|---|---|---|---|
| Tissue Name | 208122050 | Tissue Name | 208122050 |
| AD 1 Hippo | 16.4 | Control (Path) 3 Temporal | 0.0 |
| | | Ctx | |
| AD 2 Hippo | 20.6 | Control (Path) 4 Temporal | 0.0 |
| | | Ctx | |
| AD 3 Hippo | 22.8 | AD 1 Occipital Ctx | 0.0 |
| AD 4 Hippo | 0.0 | AD 2 Occipital Ctx | 0.0 |
| | | (Missing) | |
| AD 5 Hippo | 0.0 | AD 3 Occipital Ctx | 0.0 |
| AD 6 Hippo | 87.1 | AD 4 Occipital Ctx | 0.0 |
| Control 2 Hippo | 6.2 | AD 5 Occipital Ctx | 0.0 |
| Control 4 Hippo | 30.6 | AD 6 Occipital Ctx | 0.0 |
| Control (Path) 3 Hippo | 25.0 | Control 1 Occipital Ctx | 0.0 |
| AD 1 Temporal Ctx | 0.0 | Control 2 Occipital Ctx | 0.0 |
| AD 2 Temporal Ctx | 6.9 | Control 3 Occipital Ctx | 0.0 |
| AD 3 Temporal Ctx | 0.0 | Control 4 Occipital Ctx | 0.0 |
| AD 4 Temporal Ctx | 0.0 | Control (Path) 1 Occipital | 5.6 |
| | | Ctx | |
| AD 5 Inf Temporal Ctx | 0.0 | Control (Path) 2 Occipital | 0.0 |
| | | Ctx | |
| AD 5 Sup Temporal Ctx | 100.0 | Control (Path) 3 Occipital | 0.0 |
| | | Ctx | |
| AD 6 Inf Temporal Ctx | 0.0 | Control (Path) 4 Occipital | 0.0 |
| | | Ctx | |
| AD 6 Sup Temporal Ctx | 0.0 | Control 1 Parietal Ctx | 0.0 |
| Control 1 Temporal Ctx | 0.0 | Control 2 Parietal Ctx | 0.0 |
| Control 2 Temporal Ctx | 0.0 | Control 3 Parietal Ctx | 0.0 |
| Control 3 Temporal Ctx | 0.0 | Control (Path) 1 Parietal | 0.0 |
| | | Ctx | |
| Control 3 Temporal Ctx | 0.0 | Control (Path) 2 Parietal | 0.0 |
| | | Ctx | |
| Control (Path) 1 Temporal | 0.0 | Control (Path) 3 Parietal | 0.0 |
| Ctx | | Ctx | |
| Control (Path) 2 Temporal | 0.0 | Control (Path) 4 Parietal | 0.0 |
| Ctx | | Ctx | |

TABLE BYE
Panel 1.1

| Tissue Name | 109664919 | Tissue Name | 109664919 |
|---|---|---|---|
| Adrenal gland | 0.0 | Renal ca. UO-31 | 7.9 |
| Bladder | 2.3 | Renal ca. RXF 393 | 0.0 |
| Brain (amygdala) | 0.0 | Liver | 0.0 |
| Brain (cerebellum) | 0.1 | Liver (fetal) | 5.3 |
| Brain (hippocampus) | 0.0 | Liver ca. (hepatoblast) | 0.0 |
| | | HepG2 | |
| Brain (substantia nigra) | 6.9 | Lung | 0.1 |
| Brain (thalamus) | 7.2 | Lung (fetal) | 0.6 |
| Cerebral Cortex | 1.0 | Lung ca. (non-s.cell) HOP-62 | 18.4 |
| Brain (fetal) | 0.1 | Lung ca. (large cell)NCI- | 0.0 |
| | | H460 | |
| Brain (whole) | 0.1 | Lung ca. (non-s.cell) NCI- | 18.2 |
| | | H23 | |
| glio/astro U-118-MG | 0.0 | Lung ca. (non-s.cl) NCI- | 16.4 |
| | | H522 | |
| astrocytoma SF-539 | 0.7 | Lung ca. (non-sm. cell) | 8.6 |
| | | A549 | |
| astrocytoma SNB-75 | 0.0 | Lung ca. (s.cell var.) SHP- | 1.0 |
| | | 77 | |
| astrocytoma SW1783 | 0.0 | Lung ca. (small cell) LX-1 | 0.0 |
| glioma U251 | 0.0 | Lung ca. (small cell) NCI- | 57.4 |
| | | H69 | |
| glioma SF-295 | 0.0 | Lung ca. (squam.) SW 900 | 4.3 |
| glioma SNB-19 | 10.7 | Lung ca. (squam.) NCI- | 9.2 |
| | | H596 | |
| glio/astro U87-MG | 0.0 | Lymph node | 0.0 |
| neuro*; met SK-N-AS | 0.0 | Spleen | 0.0 |
| Mammary gland | 0.1 | Thymus | 0.5 |
| Breast ca. BT-549 | 1.8 | Ovary | 6.6 |
| Breast ca. MDA-N | 1.1 | Ovarian ca. IGROV-1 | 0.5 |
| Breast ca.* (pl.ef) T47D | 25.7 | Ovarian ca. OVCAR-3 | 14.2 |
| Breast ca.* (pl.ef) MCF-7 | 2.5 | Ovarian ca. OVCAR-4 | 3.2 |
| Breast ca.* (pl.ef) MDA- | 0.0 | Ovarian ca. OVCAR-5 | 51.1 |
| MB-231 | | | |
| Small intestine | 0.0 | Ovarian ca. OVCAR-8 | 6.3 |
| Colorectal | 4.0 | Ovarian ca.* (ascites) SK- | 62.4 |
| | | OV-3 | |
| Colon ca. HT29 | 5.7 | Pancreas | 4.5 |
| Colon ca. CaCo-2 | 2.3 | Pancreatic ca. CAPAN 2 | 0.0 |
| Colon ca. HCT-15 | 12.2 | Pituitary gland | 3.7 |
| Colon ca. HCT-116 | 0.0 | Placenta | 0.0 |
| Colon ca. HCC-2998 | 1.3 | Prostate | 0.0 |
| Colon ca. SW480 | 0.8 | Prostate ca.* (bone met) | 0.5 |
| | | PC-3 | |
| Colon ca.* SW620 (SW480 | 0.0 | Salivary gland | 0.6 |
| met) | | | |
| Stomach | 0.0 | Trachea | 0.0 |
| Gastric ca. (liver met) NCI- | 8.2 | Spinal cord | 10.4 |
| N87 | | | |
| Heart | 0.0 | Testis | 100.0 |
| Skeletal muscle (Fetal) | 0.3 | Thyroid | 0.0 |
| Skeletal muscle | 0.4 | Uterus | 0.0 |
| Endothelial cells | 0.1 | Melanoma M14 | 8.4 |
| Heart (Fetal) | 0.3 | Melanoma LOX IMVI | 0.0 |
| Kidney | 0.3 | Melanoma UACC-62 | 0.0 |
| Kidney (fetal) | 0.0 | Melanoma SK-MEL-28 | 1.0 |
| Renal ca. 786-0 | 2.3 | Melanoma* (met) SK- | 0.0 |
| | | MEL-5 | |
| Renal ca. A498 | 1.1 | Melanoma Hs688(A).T | 0.0 |
| Renal ca. ACHN | 0.0 | Melanoma* (met) | 0.5 |
| | | Hs688(B).T | |
| Renal ca. TK-10 | 0.2 | | |

CNS_neurodegeneration_v1.0Summary: Ag2497 A low level of expression of the CG57678-01 gene is detected with samples derived from one of the Alzheimer's patient hippocampus (CT=34.3) and sup temporal cortex (CT=34.1). Therefore, expression of this gene may be used to distinguish these samples from the other samples on this panel. This gene encodes a putative GPCR; therefore, therapeutic modulation of this gene or its protein product may be beneficial in the treatment of one or more of CNS disorders, as may stimulation and/or blockade of the receptor coded for by the gene.

Ag3306 Expression of this gene is low/undetectable (CTs>35) across all of the samples on this panel (data not shown).

General_screening_panel_v1.4 Summary: Ag3306 Expression of the CG57678-01 gene is low/undetectable (CTs>35) across all of the samples on this panel (data not shown).

Panel 1.1 Summary: Gpcr22 Expression of the CG57678-01 gene is highest in testis (CT=29.2). In addition, this gene is expressed at significant levels in some regions of the central nervous system, including spinal cord, substantia nigra and thalamus. The CG57678-01 gene encodes a putative GPCR. Several neurotransmitter receptors are GPCRs, including the dopamine receptor family, the serotonin receptor family, the GABAB receptor, muscarinic acetylcholine receptors, and others; thus this GPCR may represent a novel neurotransmitter receptor. Targeting various neurotransmitter receptors (dopamine, serotonin) has proven to be an effective therapy in psychiatric illnesses such as schizophrenia, bipolar disorder, and depression. Furthermore, the cerebral cortex and hippocampus are regions of the brain that are known to be involved in Alzheimer's disease, seizure disorders, and in the normal process of memory formation. Therefore, therapeutic modulation of this gene or its protein product may be beneficial in the treatment of one or more of these diseases, as may stimulation and/or blockade of the receptor coded for by the gene.

In addition, expression of this gene is upregulated in a number of lung cancer cell lines compared to normal lung. Therefore, therapeutic modulation of the activity of this gene or its protein product, using small molecule drugs, antibodies or protein therapeutics, may be of benefit in the treatment of lung cancer.

Panel 1.3D Summary: Ag2497 Expression of the CG57678-01 gene is low/undetectable (CTs>35) across all of the samples on this panel (data not shown).

Panel 2.2 Summary: Ag2497 Expression of the CG57678-01 gene is low/undetectable (CTs>34.5) across all of the samples on this panel (data not shown).

Panel 4D Summary: Ag2497 Expression of the CG57678-01 gene is low/undetectable (CTs>34.5) across all of the samples on this panel (data not shown).

### Example 3

### SNP Analysis of NOVX Clones

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

### Results

Variants are reported individually but any combination of all or a select subset of variants are also included as contemplated NOVX embodiments of the invention.

NOV36a SNP Data:

NOV36a has 4 SNP variants, whose variant positions for their nucleotide and amino acid sequences is numbered according to SEQ ID NOs:81 and 82, respectively. The nucleotide sequence of the NOV36a variants differs as shown in Table SNP1.

TABLE SNP1
cSNP and Coding Variants for NOV36a

| NT Position | Wild Type | | Amino Acid | Amino Acid |
|---|---|---|---|---|
| of cSNP | NT | Variant NT | position | Change |
| 284 | T | C | 87 | Phe to Ser |
| 307 | G | A | 95 | Gly to Ser |
| 354 | A | G | 110 | Ala to Ala |
| 379 | T | C | 119 | Ser to Pro |

NOV55 SNP Data:

NOV55 has 5 SNP variants, whose variant positions for their nucleotide and amino acid sequences is numbered according to SEQ ID NOs:127 and 128, respectively. The nucleotide sequence of the NOV55 variants differs as shown in Table SNP2.

TABLE SNP2
cSNP and Coding Variants for NOV55

| NT Position | Wild Type | | Amino Acid | Amino Acid |
|---|---|---|---|---|
| of cSNP | NT | Variant NT | position | Change |
| 149 | A | T | 45 | Ser to Cys |
| 227 | G | A | 71 | Glu to Lys |
| 304 | C | T | 96 | His to His |
| 2542 | T | C | 842 | His to His |
| 2838 | T | C | 941 | Ile to Thr |

NOV SNP19 Data:

NOV19 has one SNP variant, whose variant positions for their nucleotide and amino acid sequences is numbered according to SEQ ID NOs:47 and 48, respectively. The nucleotide sequence of the NOV19 variants differs as shown in Table SNP3.

TABLE SNP3
cSNP and Coding Variants for NOV19

| NT Position | Wild Type | | Amino Acid | Amino Acid |
|---|---|---|---|---|
| of cSNP | NT | Variant NT | position | Change |
| 893 | C | T | 289 | Ala to Val |

NOV8a SNP Data:

NOV8a has two SNP variants, whose variant positions for their nucleotide and amino acid sequences is numbered according to SEQ ID NOs:17 and 18, respectively. The nucleotide sequence of the NOV8a variants differs as shown in Table SNP4.

TABLE SNP4
cSNP and Coding Variants for NOV8a

| NT Position | Wild Type | | Amino Acid | Amino Acid |
|---|---|---|---|---|
| of cSNP | NT | Variant NT | position | Change |
| 1199 | T | C | 380 | Leu to Pro |
| 1213 | G | A | 385 | Gly to Ser |

NOV62a SNP Data:

NOV62a has three SNP variants, whose variant positions for their nucleotide and amino acid sequences is numbered according to SEQ ID NOs:145 and 156, respectively. The nucleotide sequence of the NOV62a variants differs as shown in Table SNP5.

TABLE SNP5
cSNP and Coding Variants for NOV62a

| NT Position | Wild Type | | Amino Acid | Amino Acid |
|---|---|---|---|---|
| of cSNP | NT | Variant NT | position | Change |
| 1197 | G | A | 366 | Ala to Thr |
| 1237 | A | G | 379 | Asp to Gly |
| 1285 | C | T | 395 | Ala to Val |

NOV SNP11a Data:

NOV11a has four SNP variants, whose variant positions for their nucleotide and amino acid sequences is numbered according to SEQ ID NOs:25 and 26, respectively. The nucleotide sequence of the NOV11a variants differs as shown in Table SNP6.

TABLE SNP6
cSNP and Coding Variants for NOV11a

| NT Position | Wild Type | | Amino Acid | Amino Acid |
|---|---|---|---|---|
| of cSNP | NT | Variant NT | position | Change |
| 636 | C | T | 212 | No change |
| 1089 | G | A | 363 | Met to Ile |
| 5654 | A | G | 1885 | Gln to Art |
| 5914 | C | A | 0 | Silent |

NOV25 SNP Data:

NOV25 has one SNP variant, whose variant positions for their nucleotide and amino acid sequences is numbered according to SEQ ID NOs:59 and 60, respectively. The nucleotide sequence of the NOV25 variants differs as shown in Table SNP7.

TABLE SNP7
cSNP and Coding Variants for NOV25

| NT Position | Wild Type | | Amino Acid | Amino Acid |
|---|---|---|---|---|
| of cSNP | NT | Variant NT | position | Change |
| 1836 | C | T | 612 | No change |

NOV10 SNP Data:

NOV10 has one SNP variant, whose variant positions for their nucleotide and amino acid sequences is numbered according to SEQ ID NOs:23 and 24, respectively. The nucleotide sequence of the NOV10 variant differs as shown in Table SNP8.

TABLE SNP8
cSNP and Coding Variants for NOV10

| NT Position | Wild Type | | Amino Acid | Amino Acid |
|---|---|---|---|---|
| of cSNP | NT | Variant NT | position | Change |
| 715 | T | C | 239 | Ser to Pro |

NOV3 SNP Data:

NOV3 has two SNP variants, whose variant positions for their nucleotide and amino acid sequences is numbered according to SEQ ID NOs:5 and 6, respectively. The nucleotide sequence of the NOV3 variants differs as shown in Table SNP9.

TABLE SNP9
cSNP and Coding Variants for NOV3

| NT Position | Wild Type | | Amino Acid | Amino Acid |
|---|---|---|---|---|
| of cSNP | NT | Variant NT | position | Change |
| 127 | C | T | 9 | No change |
| 158 | G | A | 20 | Val to Met |

NOV44a SNP Data:

NOV44a has four SNP variants, whose variant positions for their nucleotide and amino acid sequences is numbered according to SEQ ID NOs:101 and 102, respectively. The nucleotide sequence of the NOV44a variants differs as shown in Table SNP10.

TABLE SNP10
cSNP and Coding Variants for NOV44a

| NT Position | Wild Type | | Amino Acid | Amino Acid |
|---|---|---|---|---|
| of cSNP | NT | Variant NT | position | Change |
| 56 | G | T | 7 | Gly to Val |
| 164 | C | A | 43 | Pro to His |
| 422 | G | A | 129 | Gly to Asp |
| 500 | T | C | 155 | Leu to Pro |

NOV57 SNP Data:

NOV57 has one SNP variant, whose variant positions for its nucleotide and amino acid sequences is numbered according to SEQ ID NOs:131 and 132, respectively. The nucleotide sequence of the NOV57 variant differs as shown in Table SNP11.

TABLE SNP11
cSNP and Coding Variants for NOV57

| NT Position | Wild Type | | Amino Acid | Amino Acid |
|---|---|---|---|---|
| of cSNP | NT | Variant NT | position | Change |
| 1214 | C | T | 362 | No change |

NOV15 SNP Data:

NOV15 has 14 SNP variants, whose variant positions for their nucleotide and amino acid sequences is numbered according to SEQ ID NOs:39 and 40, respectively. The nucleotide sequence of the NOV15 variants differs as shown in Table SNP12.

TABLE SNP12
cSNP and Coding Variants for NOV15

| NT Position | Wild Type | | Amino Acid | Amino Acid |
|---|---|---|---|---|
| of cSNP | NT | Variant NT | position | Change |
| 136 | T | C | 11 | No change |
| 187 | T | C | 28 | No change |
| 189 | A | G | 29 | Asp to Gly |
| 259 | A | G | 52 | No change |
| 266 | C | T | 55 | Gln to stop |
| 434 | C | T | 111 | Pro to Ser |
| 998 | A | G | 299 | Ile to Val |
| 1252 | G | A | 383 | No change |
| 1409 | A | G | 436 | Thr to Ala |
| 1428 | T | C | 442 | Leu to Ser |
| 1429 | G | A | 442 | No change |
| 1935 | A | G | 611 | His to Arg |
| 1967 | G | T | 622 | Gly to Cys |
| 2063 | G | C | 654 | Ala to Pro |

NOV21 SNP Data:

NOV21 has one SNP variant, whose variant positions for its nucleotide and amino acid sequences is numbered according to SEQ ID NOs:51 and 52, respectively. The nucleotide sequence of the NOV21 variants differs as shown in Table SNP13.

TABLE SNP13
cSNP and Coding Variants for NOV21

| NT Position | Wild Type | | Amino Acid | Amino Acid |
|---|---|---|---|---|
| of cSNP | NT | Variant NT | position | Change |
| 814 | T | C | 196 | No change |

NOV45a SNP Data:

NOV45a has one SNP variant, whose variant positions for its nucleotide and amino acid sequences is numbered according to SEQ ID NOs:105 and 106, respectively. The nucleotide sequence of the NOV45a variants differs as shown in Table SNP14.

TABLE SNP14
cSNP and Coding Variants for NOV45a

| NT Position | Wild Type | | Amino Acid | Amino Acid |
|---|---|---|---|---|
| of cSNP | NT | Variant NT | position | Change |
| 4091 | A | G | 1353 | Thr to Ala |

NOV54 SNP Data:

NOV54 has one SNP variant, whose variant positions for its nucleotide and amino acid sequences is numbered according to SEQ ID NOs:125 and 126, respectively. The nucleotide sequence of the NOV54 variants differs as shown in Table SNP15.

TABLE SNP15
cSNP and Coding Variants for NOV54

| NT Position | Wild Type | | Amino Acid | Amino Acid |
|---|---|---|---|---|
| of cSNP | NT | Variant NT | position | Change |
| 3208 | C | T | Silent | No change |

NOV69a SNP Data:

NOV69a has four SNP variants, whose variant positions for their nucleotide and amino acid sequences is numbered according to SEQ ID NOs:163 and 164, respectively. The nucleotide sequence of the NOV69a variants differs as shown in Table SNP16.

TABLE SNP16
cSNP and Coding Variants for NOV69a

| NT Position | Wild Type | | Amino Acid | Amino Acid |
|---|---|---|---|---|
| of cSNP | NT | Variant NT | position | Change |
| 1052 | G | A | 327 | No change |
| 1171 | A | G | 367 | Lys to Arg |
| 1186 | A | G | 372 | Glu to Gly |
| 1316 | C | A | 415 | No change |

NOV58 SNP Data:

NOV58 has three SNP variants, whose variant positions for their nucleotide and amino acid sequences is numbered according to SEQ ID NOs:133 and 134, respectively. The nucleotide sequence of the NOV58 variants differs as shown in Table SNP17.

TABLE SNP17
cSNP and Coding Variants for NOV58

| NT Position | Wild Type | | Amino Acid | Amino Acid |
|---|---|---|---|---|
| of cSNP | NT | Variant NT | position | Change |
| 579 | C | T | 174 | Gln to Stop |
| 598 | C | T | 180 | Ala to Val |
| 637 | A | G | 193 | Asp to Gly |

### Example 4

### In-frame Cloning

NOV45b

For NOV45b, the cDNA coding for the DOMAIN of NOV45a (CG50718-02) from residues 511 to 705 was targeted for “in-frame” cloning by PCR. The PCR template was based on the previously identified plasmid, when available, or on human cDNA(s).

TABLE IFC1A
Oligonucleotide Primers Used to Clone the Target cDNA Sequence:

| Primers | Sequences | |
|---|---|---|
| F3 | 5′-AGATCT CCCCATGCCCCGGGCAGTGTCCGGGTCC-3′ | (SEQ ID NO:548) |
| R3 | 5′-CTCGAG GCTGGGCTCGCCGATCAGATCCTGCATGAC-3′ | (SEQ ID NO:549) |

For downstream cloning purposes, the forward primer includes an in-frame BglII restriction site and the reverse primer contains an in-frame Xho I restriction site.

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

TABLE IFC1B
Gene-specific Primers

| | | |
|---|---|---|
| NO | Primers | Sequences |

(SEQ ID NO:550)

| | | |
|---|---|---|
| NOV45a | SF1 | CCTGGAGCCTGAGACAGCG |

(SEQ ID NO:551)

| | |
|---|---|
| SF2 | AGCATTCCCTATTACAACTCCAGAA |

(SEQ ID NO:552)

| | |
|---|---|
| SR1 | GGTGTCCACCAGCAGCCAG |

(SEQ ID NO:553)

| | |
|---|---|
| SR2 | ATCACAGTGACCACCTCACTGAA |

NOV7

For NOV7, the cDNA coding for a domain of CG57595-01 from residue 2191 to 2450 was targeted for “in-frame” cloning by PCR. The PCR template is based on human cDNA(s).

The following oligonucleotide primers were used to clone the target cDNA sequence:

TABLE IFC2A
Oligonucleotide Primers Used to Clone the Target cDNA Sequence:

| Primers | Sequences | |
|---|---|---|
| F5 | 5′-GAATTC TGTGCAAACGGGCACCACGACTGCAACG-3′ | (SEQ ID NO:554) |
| R4 | 5′-CTCGAG GCACTGCTGGCCGCCCAGCGGACTCCCGTG-3′ | (SEQ ID NO:555) |

For downstream cloning purposes, the forward primer includes an in-frame EcoRI restriction site and the reverse primer contains an in-frame XhoI restriction site.

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

TABLE IFC2B
Gene-specific Primers

| NOV | Primers | Sequences | |
|---|---|---|---|
| NOV7 | SF1 | TTGCTCTGCCGCAACCAC | (SEQ ID NO:556) |
| | SF2 | AGCTATGGGGAGAAATGCGAG | (SEQ ID NO:557) |
| | SR1 | CAGAGCAAGCAGTGGTCACG | (SEQ ID NO:558) |
| | SR2 | TGTTATTCTGGCAGTTCACGC | (SEQ ID NO:559) |

The cDNA coding for a domain of the full length form onf CG57595-01 between residues 1885 and 2450 was targeted for “in-frame” cloning by PCR. The PCR template is based on human cDNA(s).

The following oligonucleotide primers were used to clone the target cDNA sequence:

TABLE IFC2CA
Oligonucleotide primers used to clone the target cDNA sequence:

| Primers | Sequences | |
|---|---|---|
| F5 | 5′-GAATTC TGTGCAAACGGGCACCACGACTGCAACG-3′ | (SEQ ID NO:560) |
| R4 | 5′-CTCGAG GCACTGCTGGCCGCCCAGCGGACTCCCGTG-3′ | (SEQ ID NO:561) |

For downstream cloning purposes, the forward primer includes an in-frame EcoRI restriction site and the reverse primer contains an in-frame XhoI restriction site.

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

TABLE IFC2D
Gene-specific Primers

| | Pri- | | |
|---|---|---|---|
| NOV | mers | Sequences | |
| NOV7 | SF1 | CTCTGAGAATGACTGTCGGATCA | (SEQ ID NO:562) |
| | SF2 | TACCTCCTGCCTGGACTCTAAGG | (SEQ ID NO:563) |
| | SF3 | CTCCTGGGCCTTCCTGTCCT | (SEQ ID NO:564) |
| | SF4 | CTGCAACCGCCACAGTGAAT | (SEQ ID NO:565) |
| | SF5 | CGTGTGCGTGAACTGCCAGAATA | (SEQ ID NO:566) |
| | SR1 | AGAGGTGCAGGACCCATTGC | (SEQ ID NO:567) |
| | SR2 | ACAGTTGGGTAGGAGGTGACAAG | (SEQ ID NO:568) |
| | SR3 | CCCACATGTCAGCCCATCAC | (SEQ ID NO:569) |
| | SR4 | ATTCACTGTGGCGGTTGCAG | (SEQ ID NO:570) |
| | SR5 | GGCCTCGTCTTCACTAGGACCC | (SEQ ID NO:571) |

NOV8

The cDNA coding for a domain of CG57452-01 from residue 156 to 815 was targeted for “in-frame” cloning by PCR. The PCR template is based on human cDNA(s).

The following oligonucleotide primers were used to clone the target cDNA sequence:

TABLE IFC3A
Oligonucleotide primers used to clone the target cDNA sequence:

| Primers | Sequences | |
|---|---|---|
| F2 | 5′-GGATCC TACTATGCCACAGTGAATGAGCTCACTCC-3′ | (SEQ ID NO:572) |
| R2 | 5′-CTCGAG CAAAACCTTGATGGCCAAGGTTAGAGTTGAATG-3′ | (SEQ ID NO:573) |

For downstream cloning purposes, the forward primer includes an in-frame BamHI restriction site and the reverse primer contains an in-frame XhoI restriction site.

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

TABLE IFC3B
Gene-specific Primers

| | | |
|---|---|---|
| NOV | Primers | Sequences |

(SEQ ID NO:574)

| | | |
|---|---|---|
| NOV8 | SF1 | GTTTCTTCCTTGTGTCCTTGTGCC |

(SEQ ID NO:575)

| | |
|---|---|
| SF2 | CCTTTGCCGGTCTACACATTGAAATACT |

(SEQ ID NO:576)

| | |
|---|---|
| SF3 | TTGATGGTGTACAAGAAAGTGAGCCAG |

(SEQ ID NO:577)

| | |
|---|---|
| SF4 | CGAAGGAACTCCATCTGCACTGTGTAT |

(SEQ ID NO:578)

| | |
|---|---|
| SF5 | CAGTAAACATAATGGTGACAGATGTCAATG |

(SEQ ID NO:579)

| | |
|---|---|
| SR1 | AAACATTGGACCCAAGTCATCTCC |

(SEQ ID NO:580)

| | |
|---|---|
| SR2 | GATGACCATTGTCTTGTTCAGCCTT |

(SEQ ID NO:581)

| | |
|---|---|
| SR3 | AATGCTGTTATCGAAAAGGTGTAAGTT |

(SEQ ID NO:582)

| | |
|---|---|
| SR4 | AATATACACAGTGCAGATGGAGTTCCT |

(SEQ ID NO:583)

| | |
|---|---|
| SR5 | GTTTACTGTGGCAGTTGAGGTCCCAT |

The cDNA coding for a domain of the full length CG57452-01 from residue 728 to 1382 was targeted for “in-frame” cloning by PCR. The PCR template is based human cDNA(s).

The following oligonucleotide primers were used to clone the target cDNA sequence:

TABLE IFC3C
Oligonucleotide primers used to clone the target cDNA sequence:

| Primers | Sequences | |
|---|---|---|
| F3 | 5′-GGATCC AGAAATTTATCTGTGGTGGAAGAAGAAGC-3′ | (SEQ ID NO:584) |
| R1 | 5′-CTCGAG CCCTTCTGTGTATCCTAGACTTTCTCCTC-3′ | (SEQ ID NO:585) |

For downstream cloning purposes, the forward primer includes an in-frame BamHI restriction site and the reverse primer contains an in-frame XhoI restriction site.

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

TABLE IFC3d
Gene-specific Primers

| | | |
|---|---|---|
| NOV | Primers | Sequences |

(SEQ ID NO:586)

| | | |
|---|---|---|
| NOV8 | SF1 | ACTACCATCCTTCAAATAGAGGCCA |

(SEQ ID NO:587)

| | |
|---|---|
| SF2 | CACAACAGTTTATGCTGAAGATGCAGAC |

(SEQ ID NO:588)

| | |
|---|---|
| SF3 | ATGGTTGGTGTAATTTCTGCTGCTG |

(SEQ ID NO:589)

| | |
|---|---|
| SF4 | AGAGTGAAGGCTACTGATAAAGATACTGGC |

(SEQ ID NO:590)

| | |
|---|---|
| SF5 | AAATCTTGGATCGCTATGTTCAGGA |

(SEQ ID NO:591)

| | |
|---|---|
| SR1 | TAGTCCCAGCTGGCAAATTCTCTTCAA |

(SEQ ID NO:592)

| | |
|---|---|
| SR2 | ATAGGTGTACCCTTGACTGCATCCG |

(SEQ ID NO:593)

| | |
|---|---|
| SR3 | CCTTTGGTGGCAAGTTCACTTACTG |

(SEQ ID NO:594)

| | |
|---|---|
| SR4 | CAGAAGTAAACATTCTTGCATCTTCAG |

(SEQ ID NO:595)

| | |
|---|---|
| SR5 | CATAGCGATCCAAGATTTCTGTAAGAT |

NOV11

The cDNA coding for a domain of CG57488-03 from residue 121 to 741 was targeted for “in-frame” cloning by PCR. The PCR template is based on human cDNA(s).

The following oligonucleotide primers were used to clone the target cDNA sequence:

TABLE IFC4A
Oligonucleotide primers used to clone the target cDNA sequence:

| Primers | Sequences | |
|---|---|---|
| F1 | 5′-AGATCT CAGCCTCAGGCCCCGGGTTACTTGATTGCAGC-3′ | (SEQ ID NO:596) |
| R1 | 5′-AAGCTT AGAAACATCATAATCTTCCAGTTCCTGG-3′. | (SEQ ID NO:597) |

For downstream cloning purposes, the forward primer includes an in-frame BglII restriction site and the reverse primer contains an in-frame HindIII restriction site.

TABLE IFC4B
Gene-specific Primers

| | | |
|---|---|---|
| NOV | Primers | Sequences |

(SEQ ID NO:598)

| | | |
|---|---|---|
| NOV11 | SF1 | GGACAAGCCTGTGTACAGACCCC |

(SEQ ID NO:599)

| | |
|---|---|
| SF2 | GGTATATCCAAGACCTGGACGCC |

(SEQ ID NO:600)

| | |
|---|---|
| SF3 | CAGCTGGTGGACATCCGGTACT |

(SEQ ID NO:601)

| | |
|---|---|
| SF4 | GTACTCCCCCAGCCAGTGCTACCT |

(SEQ ID NO:602)

| | |
|---|---|
| SF5 | CTCTCTTCATCTGGCCGTGACC |

(SEQ ID NO:603)

| | |
|---|---|
| SR1 | TCTGGATGAATACAGAAGCGCCC |

(SEQ ID NO:604)

| | |
|---|---|
| SR2 | GCGGGTCAATCAGAAGCTCAAAC |

(SEQ ID NO:605)

| | |
|---|---|
| SR3 | CTTGGAGTACCGGATGTCCACCAG |

(SEQ ID NO:606)

| | |
|---|---|
| SR4 | AGGGAGAGGTAGCTGGGCAGGTACTGA |

(SEQ ID NO:607)

| | |
|---|---|
| SR5 | CAGATGAAGAGAGGTCACACACACG |

The cDNA coding for a domain of the full-length protein CG57488-03 residue 119 to 741. was targeted for “in-frame” cloning by PCR. The PCR template is human cDNA(s).

The following oligonucleotide primers were used to clone the target cDNA sequence:

TABLE IFC4C
Oligonucleotide primers used to clone the target Cdna sequence:

| Primers | Sequences | |
|---|---|---|
| F1 | 5′-AGATCT CAGCCTCAGGCCCCGGGTTACTTGATTGCAGC-3′ | (SEQ ID NO:608) |
| R1 | 5′-AAGCTT AGAAACATCATAATCTTCCAGTTCCTGG-3′ | (SEQ ID NO:609) |

For downstream cloning purposes, the forward primer includes an in-frame BglII restriction site and the reverse primer contains an in-frame HindIII restriction site.

TABLE IFC4D
Gene-specific Primers

| | | |
|---|---|---|
| NOV | Primers | Sequences |

(SEQ ID NO:610)

| | | |
|---|---|---|
| NOV11 | SF1 | GGACAAGCCTGTGTACAGACCCC |

(SEQ ID NO:611)

| | |
|---|---|
| SF2 | GGTATATCCAAGACCTGGACGCC |

(SEQ ID NO:612)

| | |
|---|---|
| SF3 | CAGCTGGTGGACATCCGGTACT |

(SEQ ID NO:613)

| | |
|---|---|
| SF4 | GTACTCCCCCAGCCAGTGCTACCT |

(SEQ ID NO:614)

| | |
|---|---|
| SF5 | CTCTCTTCATCTGGCCGTGACC |

(SEQ ID NO:615)

| | |
|---|---|
| SR1 | TCTGGATGAATACAGAAGCGCCC |

(SEQ ID NO:616)

| | |
|---|---|
| SR2 | GCGGGTCAATCAGAAGCTCAAAC |

(SEQ ID NO:617)

| | |
|---|---|
| SR3 | CTTGGAGTACCGGATGTCCACCAG |

(SEQ ID NO:618)

| | |
|---|---|
| SR4 | AGGGAGAGGTAGCTGGGCAGGTACTGA |

(SEQ ID NO:619)

| | |
|---|---|
| SR5 | CAGATGAAGAGAGGTCACACACACG |

NOV52

For NOV52, the cDNA coding for the full-length NOV52 (CG57748-01) was targeted for “in-frame” cloning by PCR. The PCR template was based on the previously identified plasmid, when available, or on human cDNA(s).

TABLE IFC5A
Oligonucleotide primers used to clone the target cDNA sequence:

| Primers | Sequences | |
|---|---|---|
| F2 | 5′-AAGCTTATCTGGCAGTGTGGTGGGGTTCTGGAAAC-3′ | (SEQ ID NO:620) |
| R1 | 5′-CTCGAGTAACATGCGCTCTTTGAAGAACCATTTCTGATG-3′ | (SEQ ID NO:621) |

For downstream cloning purposes, the forward primer includes an in-frame HindIII restriction site and the reverse primer contains an in-frame Xho I restriction site.

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

TABLE IFC5B
Gene-specific Primers

| | | |
|---|---|---|
| NOV | Primers | Sequences |

(SEQ ID NO:622)

| | | |
|---|---|---|
| NOV52 | SF1 | TCTTGGAGACTGTGTATCCAGAACT |

SEQ ID NO:623)

| | |
|---|---|
| SF2 | CATTGCTGTCGGAAGACAGGAATGGAT |

(SEQ ID NO:624)

| | |
|---|---|
| SR1 | GAACCACTTGAAGTCTTTACACTGGAG |

(SEQ ID NO:625)

| | |
|---|---|
| SR2 | CAGCCCTCAGGCTGGTGGGTGTTATAG |

For NOV52, the cDNA coding for the domain of NOV52 (CG57748-01) from residue 225 to 412 was targeted for “in-frame” cloning by PCR. The PCR template was based on the previously identified plasmid, when available, or on human cDNA(s).

TABLE IFC5C
Oligonucleotide primers used to clone the target Cdna sequence:

| Primers | Sequences | |
|---|---|---|
| F2 | 5′-GGATCCATCTCGGTGTGGGTCCCGGAAAATGCACCTG-3′ | (SEQ ID NO:626) |
| R1 | 5′-CTCGAGATTAGCAAGGATGGTGAGCTTGTGGGACTCC-3′ | (SEQ ID NO:627) |

For downstream cloning purposes, the forward primer includes an in-frame BamHI restriction site and the reverse primer contains an in-frame Xho I restriction site.

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

TABLE IFC5D
Gene-specific Primers

| | Pri- | | |
|---|---|---|---|
| NOV | mers | Sequences | |
| NOV | SF1 | CAGGGAAACCCAGGACCAGTA | (SEQ ID NO:628) |
| 52 | SF2 | TCCAGGCTGTGGATAAAGACTTG | (SEQ ID NO:629) |
| | SR1 | TGTCCAAAGCACCCATGACAAT | (SEQ ID NO:630) |
| | SR2 | GACAGCTGCAATTGGGGTGC | (SEQ ID NO:631) |

NOV54

The cDNA coding for the mature form of the full length CG57707-01 from residue 23 to 543 was targeted for “in-frame” cloning by PCR. The PCR template is based on human cDNA(s).

TABLE IFC6A
Oligonucleotide primers used to clone the target
cDNA sequence:

| | |
|---|---|
| Primers | Sequences |

(SEQ ID NO:632)

| | |
|---|---|
| F1 | 5′-GGATCC CTGATACCGCGGAGCGCGCAGGTGAGG-3′ |

(SEQ ID NO:633)

| | |
|---|---|
| R3 | 5′-CTCGAGCAAACTTAAGTCAACAATTATATGTTCAAA-3′ |

For downstream cloning purposes, the forward primer includes an in-frame BamHI restriction site and the reverse primer contains an in-frame Xho I restriction site.

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

TABLE IFC6B
Gene-specific Primers

| | | |
|---|---|---|
| NOV | Primers | Sequences |

(SEQ ID NO:634)

| | | |
|---|---|---|
| NOV54 | SF1 | TGTTCATTGAAGGGAACAAAATAGAAA |

(SEQ ID NO:635)

| | |
|---|---|
| SF2 | AGGAAAAGAAGCTAAATGACGTGACCA |

(SEQ ID NO:636)

| | |
|---|---|
| SF3 | ATCAGGTCTTTGTGGTGGTAGCCC |

(SEQ ID NO:637)

| | |
|---|---|
| SF4 | ATGCGGAGTTTGTTGATATCGATGGAA |

(SEQ ID NO:638)

| | |
|---|---|
| SF5 | ATGACCCTGCAGCCCTTTTCTTTTA |

(SEQ ID NO:639)

| | |
|---|---|
| SR1 | AGGTATTCAAGATGAAAAAGTCCAGCA |

(SEQ ID NO:640)

| | |
|---|---|
| SR2 | TGATACTCTGGTGGACCAATACACAGC |

(SEQ ID NO:641)

| | |
|---|---|
| SR3 | CATCGATGAGAATGGCCTTACAGC |

(SEQ ID NO:642)

| | |
|---|---|
| SR4 | TGAACCACTCGTGCAGTGACTGGTAAG |

(SEQ ID NO:643)

| | |
|---|---|
| SR5 | GATGGAAGAGCTTGGATCTCCACAA |

NOV56

The cDNA coding for a domain of the full length CG57348-01 from residues 202 to 343 was targeted for “in-frame” cloning by PCR. The PCR template is based on human cDNA(s).

TABLE IFC7A
Oligonucleotide primers used to clone the target cDNA sequence:

| Primers | Sequences | |
|---|---|---|
| F2 | 5′-GGATCC TTGATTGAAAGTGGGAAGGAAGAAGGAATG-3′ | (SEQ ID NO:644) |
| R1 | 5′-CTCGAG ATGCTTCAGCCACGGGTGGGCTTCAATGGAAGC-3′ | (SEQ ID NO:645) |

For downstream cloning purposes, the forward primer includes an in-frame BamHI restriction site and the reverse primer contains an in-frame Xho I restriction site.

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

TABLE IFC7B
Gene-specific Primers

| | Pri- | | |
|---|---|---|---|
| NOV | mers | Sequences | |
| NOV | SF1 | CTCTGTATGCACAGGACCCTTCC | (SEQ ID NO:646) |
| 56 | SF2 | CAAACTGCACGACATCGACGG | (SEQ ID NO:647) |
| | SR1 | CTCCCGTTTCTTGGCGTCG | (SEQ ID NO:648) |
| | SR2 | GGTTTGGCAGTTCCCACATT | (SEQ ID NO:649) |

NOV60

The cDNA coding for a domain fo the full length CG57574-01 from residue 18 to 275 was targeted for “in-frame” cloning by PCR. The PCR template is based on human cDNA(s).

TABLE IFC8A
Oligonucleotide primers used to clone the target cDNA sequence:

| Primers | Sequences | |
|---|---|---|
| F1 | 5′-GGATCC AAATCGTGTGCTCCAAATAAAGCAGATGTCATTC-3′ | (SEQ ID NO:650) |
| R1 | 5′-CTCGAG TCCCCCGGTCTGGTCTCGCAGGAGGCG-3′ | (SEQ ID NO:651) |

For downstream cloning purposes, the forward primer includes an in-frame BamHI restriction site and the reverse primer contains an in-frame Xho I restriction site.

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

TABLE IFC8B
Gene-specific Primers

| | | |
|---|---|---|
| NOV | Primers | Sequences |

(SEQ ID NO:652)

| | | |
|---|---|---|
| NOV60 | SF1 | AAAAACCAGCCTGTCAACTACTCCTTCTC |

(SEQ ID NO:653)

| | |
|---|---|
| SF2 | ACTTCATGTATCCCTTGCAGTGGC |

(SEQ ID NO:654)

| | |
|---|---|
| SR1 | GTGGTGTCATTGGAAACGATGTG |

(SEQ ID NO:655)

| | |
|---|---|
| SR2 | ATACATGAAGTCAGCCGAGGGGGT |

Two parallel PCR reactions were set up using a total of 0.5-1.0 ng human pooled cDNAs as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus.

When the tissue of expression is known and available, the second PCR was performed using the above primers and 0.5 ng-1.0 ng of one of the following human tissue cDNAs:

skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus.

The reaction mixtures contained 2 microliters of each of the primers (original concentration: 5 pmol/ul), 1 microliter of 10 mM dNTP (Clontech Laboratories, Palo Alto Calif.) and 1 microliter of 50×Advantage-HF 2 polymerase (Clontech Laboratories) in 50 microliter-reaction volume. The following reaction conditions were used:

PCR condition 1:

a) 96° C. 3 minutes

b) 96° C. 30 seconds denaturation

c) 60° C. 30 seconds, primer annealing

d) 72° C. 6 minutes extension

Repeat steps b-d 15 times

e) 96° C. 15 seconds denaturation

f) 60° C. 30 seconds, primer annealing

g) 72° C. 6 minutes extension

Repeat steps e-g 29 times

e) 72° C. 10 minutes final extension

PCR condition 2:

a) 96° C. 3 minutes

b) 96° C. 15 seconds denaturation

c) 76° C. 30 seconds, primer annealing, reducing the temperature by 1° C. per cycle

d) 72° C. 4 minutes extension

Repeat steps b-d 34 times

e) 72° C. 10 minutes final extension

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NO:2n, wherein n is any integer 1-107;
(b) a variant of a mature form of an amino acid sequence selected from the group consisting of SEQ ID NO:2n, wherein n is any integer 1-107, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO:2n, wherein n is any integer 1-107; and
(d) a variant of an amino acid sequence selected from the group consisting of SEQ ID NO:2n, wherein n is any integer 1-107, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NO:2n, wherein n is any integer 1-107.

3. The polypeptide of claim 2, wherein said allelic variant comprises an amino acid sequence that is the translation of a nucleic acid sequence differing by a single nucleotide from a nucleic acid sequence selected from the group consisting of SEQ ID NO:2n-1, wherein n is any integer 1-107.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NO:2n, wherein n is any integer 1-107;
(b) a variant of a mature form of an amino acid sequence selected from the group consisting of SEQ ID NO:2n, wherein n is any integer 1-107, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO:2n, wherein n is any integer 1-107;
(d) a variant of an amino acid sequence selected from the group consisting SEQ ID NO:2n, wherein n is any integer 1-107, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NO:2n, wherein n is any integer 1-107, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NO:2n-1, wherein n is any integer 1-107.

9. The nucleic acid molecule of claim 5, wherein said nucleic acid molecule comprises a nucleotide sequence selected from the group consisting of:
(a) a nucleotide sequence selected from the group consisting of SEQ ID NO:2n-1, wherein n is any integer 1-107;
(b) a nucleotide sequence differing by one or more nucleotides from a nucleotide sequence selected from the group consisting of SEQ ID NO:2n-1, wherein n is any integer 1-107, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NO:2n-1, wherein n is any integer 1-107, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to a cancer.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NO:2n, wherein n is any integer 1-107, or a biologically active fragment thereof.

49. A method of treating a pathological state in a mammal, the method comprising administering to the mammal the antibody of claim 15 in an amount sufficient to alleviate the pathological state.
